(19) United States
(12) Patent Application Publication
(10) Pub. No.: US 2026/0286442 A1
(43) Pub. Date: Sep. 24, 2026

(54) Method of Diagnosing Bladder Cancer

(71) Applicant: Erasmus University Medical Center Rotterdam, Rotterdam (NL)

(72) Inventors: Ellen Catharina Zwarthoff, Rotterdam (NL); Annechiena Geertruide Van Tilborg, Gouda (NL)

(73) Assignee: Erasmus University Medical Center Rotterdam, Rotterdam (NL)

(21) Appl. No.: 19/423,276

(22) Filed: Dec. 17, 2025

Related U.S. Application Data
(63) Continuation of application No. 17/571,248, filed on Jan. 7, 2022, now Pat. No. 12,529,110.
Application No. 17/571,248 is a continuation of application No. 15/076,544, filed on Mar. 21, 2016, now Pat. No. 11,220,714.
Application No. 15/076,544 is a division of application No. 13/265,376, filed on Apr. 20, 2010, now abandoned.
(60) Provisional application No. 61/170,928, filed on Apr. 20, 2009.

(51) Int. Cl. C12Q 1/6886

(52) U.S. Cl. CPC C12Q 1/6886; C12Q 2600/154

(57) ABSTRACT

The present invention relates to a method of diagnosing cancer in a subject comprising detecting in the DNA of said subject at least one hypermethylated CpG island associated with said cancer, wherein an elevation in the level of methylation in said CpG island of said subject, relative to the level of methylation in said CpG island of a control subject, is indicative of said CpG island being hypermethylated.

### CROSS-REFERENCE TO RELATED APPLICATION

This application is a continuation of U.S. application Ser. No. 17/571,248, filed Jan. 7, 2022, which is a continuation of U.S. application Ser. No. 15/076,544, filed Mar. 21, 2016, which is a divisional of U.S. application Ser. No. 13/265,376 having an international filing date of 20 Apr. 2010 (now abandoned), which is a national phase entry of PCT application PCT/NL2010/050213 having an international filing date of 20 Apr. 2010, which claims benefit under 35 U.S.C. § 119 (e) of provisional application U.S. Application No. 61/170,928 filed 20 Apr. 2009. The contents of these documents are incorporated herein by reference.

### REFERENCE TO A SEQUENCE LISTING SUBMITTED VIA EFS-WEB

This application contains a Sequence Listing, which has been submitted electronically in XML format. The XML file entitled “01348-0004-03US.xml” was created on Dec. 16, 2025, and is 680,922 bytes in size. The Sequence Listing is incorporated herein by reference in its entirety.

### FIELD OF THE INVENTION

The present invention is in the field of bladder cancer diagnosis, and in particular the methods for predicting the progression of bladder cancer tumors. The invention provides diagnostic methods and diagnostic compositions for use in such methods. The invention further provides therapeutic targets for treating specific forms of bladder cancers.

### BACKGROUND OF THE INVENTION

Bladder cancer is the fifth most common cancer in the western world with an incidence of 20 new cases per year per 100,000 people in the U.S. Unfortunately, these statistics do not include superficial pTa bladder cancer, which represents the most common type of bladder cancer. In the Netherlands, the incidence of both superficial and invasive bladder cancer is estimated as about 30 new cases per year per 100,000 people. This is in accordance with data from global cancer statistics for the western world. Superficial bladder tumors are removed by transurethral resection. However, up to 70% of these patients will develop one or more recurrences, and it has been estimated that 1 in 1,450 people is under surveillance for bladder cancer in the United Kingdom. Cystoscopy is an uncomfortable, invasive, and expensive procedure, but currently remains the gold standard for detection of recurrences. Because patients have to be monitored perpetually and have a long-term survival, bladder cancer is the most expensive cancer when calculated on a per patient basis.

Hence there exists a need for an inexpensive, noninvasive, and simple procedure for the detection of bladder cancer. Cytology done on voided urine is a noninvasive procedure with up to 100% specificity. Unfortunately, this method is limited by its sensitivity, which is especially poor for low-grade tumors. Because of this limited sensitivity, alternative methods need to be developed for the detection of tumor cells in voided urine.

### SUMMARY OF THE INVENTION

The present inventors have found that the methylation of a number of specific CpG islands (CGIs) in the DNA of cells shaded in the urine of a subject is indicative of the present of a tumor, in particular a bladder tumor. Thus, aberrant methylation of these CpG islands in the DNA of a subject may be used as diagnostic and/or prognostic marker as well as a therapeutic target.

The present finding has been found to be particularly advantageous in the case of bladder cancer diagnostics (including disease prognosis and prediction of disease recurrence and progression), since the detection of methylation may occur in DNA (of cells) in a sample of urine obtained from the subject to be diagnosed. In particular urothelial cells present in said urine sample are used for this purpose. The skilled person will understand that diagnostic methods as indicated herein may make use of any body sample in which DNA comprising the aberrant methylations as defined herein can be detected, including biopsies of cancerous tissue, or tissue suspected of being malignant.

The present inventors have demonstrated the suitability of using methylation of specified CpG islands as diagnostic markers in bladder cancer diagnostics, such as for:
detection of recurrent cancer in DNA isolated from patient urine, and
detection of primary cancer in DNA isolated from patient urine.

Thus, in a first aspect, the present invention provides a method for diagnosing cancer in a subject comprising detecting in the DNA of said subject at least one hypermethylated CpG island associated with said cancer, wherein an elevation in the level of methylation in said CpG island of said subject, relative to the level of methylation in said CpG island of a control subject, is indicative of said CpG island being hypermethylated. The presence of a hypermethylated CpG island might indicate that the subject is at risk of, or suffering from, a cancer.

Preferably said CpG island is selected from the group consisting of the CpG islands listed in Table 1, Table 2, Table 3, Table 4, Table 5, Table 7, Table 9, Table 10 and Table 12.

In a preferred embodiment of methods of the invention, said cancer is bladder cancer.

In another preferred embodiment of methods of the invention, said method comprises detection of recurrent forms of cancer in DNA isolated from patient urine and wherein said at least one CpG islands methylation associated with cancer selected from the group consisting of the CpG islands listed in Table 1, Table 2, Table 3, Table 4, Table 5, Table 7, Table 9, Table 10 and Table 12.

In yet another preferred embodiment of methods of the invention, said method comprises detection of primary forms of cancer in DNA isolated from patient urine and wherein said at least one CpG islands methylation associated with cancer selected from the group consisting of the CpG islands listed in Table 1, Table 2, Table 3, Table 4, Table 5, Table 7, Table 9, Table 10 and Table 12.

In still a further preferred embodiment of methods of the invention, said DNA is obtained from a urine sample of said subject.

In another preferred embodiment of methods of the invention, said bladder cancer is non muscle-invasive bladder cancer. Alternatively, said bladder cancer is muscle-invasive bladder cancer. In a further preferred embodiment, said method provides detection of differential methylation of one or more of the genes provided in Table 7. Differential methylation of one or more of the genes provided in Table 7 is used to discriminate between subgroups of bladder cancer, such as between NMI-wt and MI and NMI-mt groups.

In a preferred embodiment of methods of the invention, general inflammatory cells in urine, preferably lymphocytes, are detected by methylation of at least one of the CGIs listed in Table 9. Table 9 indicates a number of CGIs methylated in blood but not in bladder cancer. The differential methylation of these CGIs, which can be detected in DNA (of cells) in a sample of urine obtained from the subject to be diagnosed, and in particular in urothelial cells present in said urine sample, are useful for the detection of lymphocytes in urine, or in general inflammatory cells in urine, which is indicative of cystitis. Positive detection of the presence of inflammatory cells in the urine can be used to diagnose infection or non-infective inflammation of the urinary tract and kidney.

In a preferred embodiment, said method comprises detection of recurrent cancer in DNA isolated from patient urine. This method entails the detection of specific CpG island methylations associated with recurrent forms of the cancer.

In another preferred embodiment, said method comprises detection of primary cancer in DNA isolated from patient urine. This method entails the detection of specific CpG island methylations associated with primary forms of the cancer.

The present inventors have further demonstrated the suitability of using methylation of specified CpG islands as prognostic markers in bladder cancer diagnostics, such as for:
prediction of disease course based on analysis of tumor-derived or urine derived DNA, including:
   prediction of invasive potential of non-muscle invasive urothelial carcinomas;
   prediction of urothelial carcinoma-specific survival, and
   prediction of presence/absence of lymph node and distant metastases caused by urothelial carcinomas

Thus, in a second aspect, the present invention provides a method for the prediction of the recurrence, progression or prognosis of cancer, in particular bladder cancer, comprising detecting in the DNA of said subject at least one hypermethylated CpG island associated with said cancer, wherein an elevation in the level of methylation in said CpG island of said subject, relative to the level of methylation in said CpG island of a control subject, is indicative of said CpG island being hypermethylated. The presence of a hypermethylated CpG island might indicate that the subject is at risk of cancer recurrence, progression of a cancer, and/or poor prognosis. A preferred method for the prognosis of a risk of cancer recurrence, progression of a cancer, and/or poor prognosis comprises detection of one or more methylated CpGs in CGIs of the genes listed in Table 12B, whereby the CGIs are preferably chosen from GPR103, DBC1 and/or GATA2 genes. Said cancer is preferably bladder cancer.

In a preferred embodiment of said method for the prediction of the recurrence, progression or prognosis of cancer, said CpG island is selected from the group consisting of the CpG islands listed in Table 1, Table 2, Table 3, Table 4, Table 5, Table 7, Table 9, Table 10 and Table 12, preferably from Table 5 and Table 10.

In aspects of the present invention the methylation of dinucleotide in or methylation level of a CpG island may be determined by any method available to the skilled person. Preferably said method comprises methylation-specific PCR (MSP). Alternatively, or additionally, use may be made of a multiplex ligation-dependent probe amplification (MLPA).

The present inventors have further demonstrated the suitability of using methylation of specified CpG islands as markers in bladder cancer diagnostics for prediction of therapeutic response, such as for prediction of failure or success of (neo)-adjuvant therapies.

The present inventors now envision as part of their invention the use of demethylating agents capable of specifically demethylating the CpG islands associated with tumor, in particular bladder cancer, as defined herein. They may also be of use as priming therapy for chemotherapeutic regimes.

Hence, the present invention provides a method for the treatment or prevention of cancer, in particular bladder cancer, comprising administering to a subject in need of such treatment a therapeutically effective amount of a demethylating agent capable of selectively demethylating at least one of the CpG islands methylations associated with cancer as described herein.

Clinical outcome of demethylating agents may depend on specific methylation profiles.

The present invention further provides pharmaceutical compositions comprising demethylating agents capable of selectively demethylating at least one of the CpG islands methylations associated with cancer as described herein.

The present invention further provides diagnostic compositions comprising PCR primirs and reagent or probes capable of selectively hybridising under stringent conditions to hypermethylated CpG islands as defined herein.

### DETAILED DESCRIPTION OF THE INVENTION

As used herein the term “CpG island” refers to genomic regions that contain a high frequency of CG nucleotides. In mammalian genomes, CpG islands are typically 300-3,000 base pairs in length. They are in and near approximately 40% of promoters of mammalian genes (about 70% in human promoters). The “p” in CpG notation refers to the phosphodiester bond between the cytosine and the guanine. CpG islands are characterized by CpG dinucleotide content of at least 60% of that which would be statistically expected (~4-6%), whereas the rest of the genome has much lower CpG frequency (~1%), a phenomenon called CG suppression. Unlike CpG sites in the coding region of a gene, in most instances, the CpG sites in the CpG islands of promoters are unmethylated if genes are expressed.

As used herein the term “CpG island methylation” refers to a methylation of at least one CG dinucleotide in a CpG region as defined herein. Hence, the methods of the present invention include the detection of at least a single CG dinucleotide being methylated in a CpG island as defined herein.

As used herein the term “hypermethylated CpG islands” refers to a CpG island exhibiting an elevation in the level of methylation, relative to the level of methylation in the same CpG island of a control subject. Again, the level of methylation includes the presence of a single methylated CG dinucleotide in the CpG island as compared to the control situation, in which the same CpG island is unmethylated. Also, methods of the invention include embodiments wherein the methylation of a single CG dinucleotide in a CpG island as defined herein is detected among other methylated sites.

As used herein, “cancer” shall be taken to mean any one or more of a wide range of benign or malignant tumors, including those that are capable of invasive growth and metastasis through a human or animal body or a part thereof, such as, for example, via the lymphatic system and/or the blood stream. As used herein, the term “tumor” includes both benign and malignant tumors or solid growths, notwithstanding that the present invention is particularly directed to the diagnosis or detection of malignant tumors and solid cancers. Typical cancers include but are not limited to carcinomas, lymphomas, or sarcomas, such as, for example, ovarian cancer, colon cancer, breast cancer, pancreatic cancer, lung cancer, prostate cancer, urinary tract cancer, uterine cancer, acute lymphatic leukemia, Hodgkin's disease, small cell carcinoma of the lung, melanoma, neuroblastoma, glioma, and soft tissue sarcoma of humans; and lymphoma, melanoma, sarcoma, and adenocarcinoma of animals. The term “bladder cancer” as used herein refers in general to urothelial cell

carcinoma, i.e. carcinomas of the urinary bladder, ureter, renal pelvis and urethra. The term includes reference to the non muscle-invasive (NMI) or superficial forms, as well as to the more dangerous muscle invasive (MI) types. Also included in the term is reference to the primary forms as well as to recurrent forms of the cancer.

The NMI types may further be subdivided into FGFR3⁺ and FGFR3⁻. Mutations in the fibroblast growth factor receptor 3 (FGFR3, indicated herein as FGFR3⁺) occur in 50% of primary bladder tumors. It is known (e.g. Van Oers et al. 2005 Clin. Cancer Res; 11(21), pp. 7743-7748) that an FGFR3 mutation is associated with good prognosis, illustrated by a significantly lower percentage of patients with progression and disease-specific mortality. FGFR3 mutations are especially prevalent in low grade/stage tumors, with pTa tumors harboring mutations in 85% of the cases. These tumors recur in 70% of patients. Efficient FGFR3 mutation detection for prognostic purposes and for detection of recurrences in urine is an important clinical issue. Thus, in preferred embodiments of the present invention the method of diagnosis includes further the step of detecting an FGRF3 gene mutation. This additional step may be performed prior to, during or after the step of detecting the hypermethylated CpG island as indicated herein. The detection of the FGFR3 gene mutation as indicated in Van Oers et al (this reference is expressly referred to and incorporated herein by reference in its entirety for the methods and mutations to be detected as described therein) indicates the cancer is very likely of the non invasive type.

“Subject” as used herein includes, but is not limited to, mammals, including, e.g., a human, a non-human primate, a mouse, a pig, a cow, a goat, a cat, a rabbit, a rat, a guinea pig, a hamster, a degu, a horse, a monkey, a sheep, or other non-human mammal; and non-mammal animals, including, e.g., a non-mammalian vertebrate, such as a bird (e.g., a chicken or duck) or a fish, and an invertebrate.

A method of the invention can be performed on any suitable body fluid, such as for instance on abdominal fluid, pleural fluid, bronchial fluid, pericardial fluid, blood, serum, milk, plasma, sweat, tears, urine, peritoneal fluid, lymph, vaginal secretion, semen, spinal fluid, cerebrospinal fluid, ascitic fluid, saliva, stool, sputum, mucus or breast exudate. Preferably, a method of the invention is performed on blood, serum, plasma or ascitic fluid, more preferably serum. Depending on the methods employed, the skilled person will be capable of establishing the amount of sample required to perform the various steps of the method of the present invention. Generally, such amounts will comprise a volume ranging from 0.01 μl to 100 ml or more. Preferred samples are urine samples.

In even more preferred embodiments of aspects of the invention the cell fraction of urine, preferably tumor cell fraction, is used. Urine cell fractions may be obtained by filtration or centrifugation. The cellular DNA may subsequently be analyzed, for instance upon extraction of the DNA from the cells, although in situ methods performed on whole cells are also envisioned.

The skilled physician or biologist will be familiar with the various ways of providing a sample of a body fluid from a subject, in particular an urine sample. Urine collection and preparation of the sample for CpG island methylation analysis suitably comprises for instance the collection of a urine sample were collected into a 50 ml centrifuge tube. The sample may optionally be used for additional tests, such as dip stick tests for leucocytes, erythrocytes and nitrite as routinely performed in the art. The cells in the urine are suitably spun down for 10 min @ 3000 rpm; 4° C. An amount of supernatant is suitably stored at-80° C. for later use. The cell pellet may then be washed, for instance by adding a suitable amount of PBS (phosphate-buffered saline), such as 10 ml, to the cell pellet. After mixing and centrifugation (e.g. 10 min @ 3000 rpm; 4° C.), the PBS may be discarded. The cell pellet may then be resuspended in PBS (e.g. 1 ml) and transferred to a microcentrifuge tube. The suspension is then suitably centrifuged (e.g. 5 min @ 6000 rpm; 4° C.). The supernatant can then be carefully removed and the cell pellet may be stored in a −80° C. freezer until DNA extraction.

Commercial systems for DNA isolation from blood, urine and tissue are available form various suppliers of molecular biological reagents. Special reference is made to the DNeasy® Blood & Tissue Kit and DNeasy® 96 Blood & Tissue Kit available from QIAGEN GmbH, Hilden, Germany.

The term “therapeutically effective amount” as used herein refers to an amount of a therapeutic agent to treat, ameliorate, or prevent a desired disease or condition, or to exhibit a detectable therapeutic or preventative effect. The effect can be detected by, for example, chemical markers or antigen levels. Therapeutic effects also include reduction in physical symptoms. The precise effective amount for a subject will depend upon the subject's size and health, the nature and extent of the condition, and the therapeutics or combination of therapeutics selected for administration. Thus, it is not useful to specify an exact effective amount in advance. However, the effective amount for a given situation can be determined by routine experimentation and is within the judgment of the clinician.

For purposes of the present invention, an effective dose will be from about 0.01 mg/kg to 50 mg/kg or 0.05 mg/kg to about 10 mg/kg of the polynucleotide or polypeptide compositions in the individual to which it is administered.

The present invention also pertains to pharmaceutical compositions comprising demethylating agents as defined herein.

A pharmaceutical composition can also contain a pharmaceutically acceptable carrier. The term “pharmaceutically acceptable carrier” refers to a carrier for administration of a therapeutic agent, such as a polypeptide, polynucleotide, and other therapeutic agents. The term refers to any pharmaceutical carrier that does not itself induce the production of antibodies harmful to the individual receiving the composition, and which may be administered without undue toxicity. Suitable carriers may be large, slowly metabolized macromolecules such as proteins, polysaccharides, polylactic acids, polyglycolic acids, polymeric amino acids, amino acid copolymers, and inactive virus particles. Such carriers are well known to those of ordinary skill in the art.

Pharmaceutically acceptable salts can be used therein, for example, mineral acid salts such as hydrochlorides, hydrobromides, phosphates, sulfates, and the like; and the salts of organic acids such as acetates, propionates, malonates, benzoates, and the like. A thorough discussion of pharmaceutically acceptable excipients is available in Remington's Pharmaceutical Sciences (Mack Pub. Co., N.J. 1991).

Pharmaceutically acceptable carriers in therapeutic compositions may contain liquids such as water, saline, glycerol and ethanol. Additionally, auxiliary substances, such as wetting or emulsifying agents, pH buffering substances, and the like, may be present in such vehicles. Typically, the therapeutic compositions are prepared as injectables, either as liquid solutions or suspensions; solid forms suitable for solution in, or suspension in, liquid vehicles prior to injection may also be prepared. Liposomes are included within the definition of a pharmaceutically acceptable carrier.

Once formulated, the pharmaceutical compositions of the invention can be (1) administered directly to the subject; (2) delivered ex vivo, to cells derived from the subject; or (3) delivered in vitro for expression of recombinant proteins.

Direct delivery of the compositions will generally be accomplished by injection, either subcutaneously, intraperitoneally, intravenously or intramuscularly, or delivered to the interstitial space of a tissue. The compositions can also be administered into the nervous system. Other modes of administration include topical, oral, suppositories, and transdermal applications, needles, and particle guns or hyposprays. Dosage treatment may be a single dose schedule or a multiple dose schedule.

Specific aspects of the present invention relate to the CpG islands indicative of certain forms of cancer emerging for instance from the following comparisons:
A) CpG islands hypermethylated in non muscle-invasive (NMI) bladder cancer (BC) that are FGFR3 wild type, herein referred to as NMI-BC wt, vs. muscle-invasive (MI) bladder cancer herein referred to as MI-BC (n=31)
B) CpG islands hypermethylated in MI-BC vs. NMI-BC wt (n=11)
C) CpG islands hypermethylated in all bladder cancers (n=62)
D) CpG islands hypermethylated in NMI-BC-FGFR3 wild type vs. NMI-BC-FGFR3 mutant (n=31), and
E) Hypermethylated CpG islands predicting progression of disease or death of disease (resp. n=3 and n=11)).

The methods of the invention comprise the detection of hypermethylated CpG islands in the DNA of a subject. As indicated, hypermethylation is a relative term indicating that the methylation level of the respective CpG island is higher than or elevated with respect to the methylation level of the corresponding CpG island in a healthy control subject.

The methods of the invention comprise the detection of at least one hypermethylated CpG island in the DNA of a subject. Since there are many CpG island indicated herein whose hypermethylation is indicative of cancer, in particular bladder cancer, the method preferably entails the detection of at least 2, 3, 4, 5, 6, 7, 8, 9, 10, 11, 12, 13, 14, 15, 16, 17, 18, 19, 20, 21, 23, 24, 25, 26, 27, 28, 29, 30, or more, such as 35, 40, 45, 50, 55, 60, 65 or more hypermethylations in the DNA of a subject. In such instances, it is possible to speak of a CpG island methylation profile. In such a profile, the percentage of positively detected CpG islands is preferably at least 50%, more preferably at least 70%, more preferably at least 90%.

The CpG island methylation profiles may comprise of between 2-25 CpG islands for diagnostic purpose, such as determining whether a bladder cancer is present, and/or whether it is an NMI or MI type.

The CpG island methylation profiles preferably comprise of between 2-25, more preferably more, such as between 50 and 150, for instance about 100 CpG islands for diagnostic purpose, such as determining the prognosis of an NMI to develop into a more aggressive MI type of tumor. Thus, the present invention provides for a prognostic method for determining whether an NMI type bladder cancer can develop into an MI type.

It is also possible to use a method of the present invention to distinguish between a primary or recurrent form of the tumor.

The skilled person is well aware of the various methods that have been developed to analyze DNA methylation. Amongst these methods are restriction enzyme- and sodium bisulfite based approaches, as well as multiplex ligation-dependent probe amplification (MLPA) approaches.

Restriction-enzyme based methods are based on the inability of methylation sensitive restriction enzymes to cleave methylated cytosines in their recognition site. The identification of the methylation status relies on Southern hybridization techniques or PCR and is based on the length of the digested DNA fragment. The inability to digest methylated sequences results in longer fragments, indicating a methylated CpG dinucleotide. Restriction-enzyme based methods are simple, rapid and highly sensitive and are suitable for genome-wide methylation analyses as well as marker discovery techniques.

Preferred methods for application in aspects of the present invention is sodium bisulfite (NaHSO₃) based detection of DNA methylation. Such methods are essentially based on the fact that treatment of single-stranded DNA with sodium bisulfite results in sequence differences due to deamination of unmethylated cytosines to uracil under conditions whereby methylated cytosines remain unchanged. The difference in methylation status marked by bisulfite reactivity can accurately be determined and quantified by PCR-based technology. Bisulfite sequencing techniques (such as described in Frommer et al. Proc. Natl. Acad. Sci. USA 89 (1992), 1827-1831) provide qualitative data on the methylation status of 5-methylcytosines in the amplicon between the sequence primers and thus requires primers specific for bisulfite converted, but not specific for unmethylated or methylated DNA. This approach provides detailed information on the methylation status of all CpG-sites.

A methylation-specific PCR (MSP) assay for determining the methylation status of CpG islands may be used. Such methods are for instance described in Herman et al. 1996 (Proc. Natl. Acad. Sci. USA 93 (1996), 9821-9826) and Herman and Baylin (Current Protocols in Human Genetics 10 (1998), 10.16.11-10.16.10). The MSP assay is based on the use of two distinct methylation-specific primer sets for the sequence of interest. The unmethylated (U) primer only amplifies sodium bisulfite converted DNA in unmethylated condition, while the methylated (M) primer is specific for sodium bisulfite converted methylated DNA. Using MSP, 1 methylated allele may be detected in a background of 1000 unmethylated alleles. MSP is very suitable for analyzing the methylation status of CpG dinucleotides in a CpG-island. The method may also be applied for high-throughput analysis of clinical samples. The most critical parameters determining the success and specificity of MSP, i.e. bisulfite conversion, primer design and PCR as well as several post PCR validation approaches are discussed in Derks et al. 2004 (Cellular Oncology 26 (2004) 291-299). Methylation Specific PCR (MSP) is described in great detail in U.S. Pat. Nos. 5,786,146, 6,017,704, 6,200,756 and 6,265,171 and International Patent WO 97/46705.

Commercial systems for DNA methylation analysis using MSP are available form various suppliers of molecular biological reagents. Special reference is made to the EZ DNA Methylation-Startup™ Kit and EZ DNA Methylation-Direct™ Kit available from Zymo Research Corp. Orange, CA, U.S.A.

Use can also be made, alone, or in combination with any of the above, of the MLPA technique. The MLPA technique is for instance described in great detail in Schouten et al., 2002 (Nucleic Acids Research 3(12) e57, 13 pp.).

Also, use can be made of a microarray chip to which DNA of a subject is hybridized. Such arrays for CpG islands are for instance commercially available from Agilent Technologies Inc., Santa Clara, USA.

Herein below are provided the following Tables:

Table 1. Coordinates of selected regions containing relevant CpG islands based on Homo sapiens full genome as provided by UCSC (hg18, March 2006). These CpG islands are aspect of the present invention as hypermethylation of these regions is associated with cancer, preferably bladder cancer and/or certain forms of said cancer.

Table 2. CpG islands differentially methylated in NMI-BC wt vs. invasive BC. These 42 CpG islands are aspect of the present invention as hypermethylation of these regions is associated with either NMI or more aggressive MI forms of the cancer. Hence, these regions are particularly indicated for use in diagnostic methods wherein typing of the cancer is relevant. The table provides data on CpG islands hypermethylated in non muscle-invasive (NMI) bladder cancer (BC) that are FGFR3 wild type, herein referred to as NMI-BC wt, vs. muscle-invasive (MI) bladder cancer herein referred to as MI-BC (n=31) as well as CpG islands hypermethylated in MI-BC vs. NMI-BC wt (n=11).

Table 3. CpG islands hypermethylated in (bladder) cancer. These 62 CpG islands are aspect of the present invention as hypermethylation of these CpG islands is associated with (bladder) cancer.

Table 4. CpG islands hypermethylated in NMI-BC FGFR3 wt vs NMI-BC FGFR3 mut. These 31 CpG islands are aspect of the present invention diagnostic markers for typing of (bladder) cancers and in particular indicate the relevance of FGFR3 mutations in bladder cancer. These 31 CpG islands may be used for diagnosis of FGFR3 wt primary and recurrent tumors in for instance urine. These 31 CpG islands may also be used for detection of FGFR3 wt tumors in patients with a FGFR3 mt (mutant) primary tumor. Alternatively, these 31 CpG islands may be used for the identification of recurrences that progressed to muscle-invasive disease or to stage pT1 or to high grade (WHO 2004 grading system) or G3 (WHO grading system 1973). These 31 CpG islands may also be used to present specific targets for therapy in NMI-BC tumors that are FGFR3 wt.

Table 5. CpG islands potentially involved in progression and death of disease. These 14 CpG islands are aspect of the present invention as hypermethylation of these regions is associated with prediction of the recurrence, progression or prognosis of cancer of cancer, in particular bladder cancer.

Table 7 (including Tables 7A and 7B) indicates differentially labeled CGIs between subgroups of bladder cancer and/or between subgroups of bladder cancer and a reference sample (i.e. blood).

Table 9 indicates a number of CGIs methylated in blood but not in bladder cancer. The differential methylation of these CGIs, which can be detected in DNA (of cells) in a sample of urine obtained from the subject to be diagnosed, and in particular in urothelial cells present in said urine sample, are—apart from the purpose indicated herein for diagnosing bladder cancer—also useful for the detection of lymphocytes in urine, or in general inflammatory cells in urine, which is indicative of cystitis. Positive detection of the presence of inflammatory cells in the urine can be used to diagnose infection or non-infective inflammation of the urinary tract and kidney.

Table 10 provides a list of hypermethylated CGIs that show 3 or more CpG dinucleotides highly methylated in a specific CGI. This list comprises 82 CGIs representing 71 genes and 11 CGIs not directly associated with a gene. A preferred gene from Table 10 is MEIS1, showing the highest degree of methylation with a fold change of 4.8 and an average fold change of 2.2 across the 10 probes that were present on the array for this CGI.

Table 12 (including Tables 12A and 12B) provides a list of CGIs that are differentially methylated between tumors and urine from non-bladder cancer control. These genes represent potential biomarkers for urine tests.

### LEGEND TO THE FIGURES

FIG. 1A-FIG. 1F. Methylation pattern and expression of selected genes. (a) MEISI, (b) NR4A2 and (c) HOXA9. (d), (e), (f) are Box plots representing the expression profiles which were taken from oncomine.

FIG. 2A-FIG. 2B. Comparison of differential methylation between subgroups. (a) Unsupervised analysis performed on all probes present on the 244K array without selection: PCA showing the distinct bladder tumor groups cluster separately (Red-MI, Blue-NMI-mt and Green NMI-wt), (b) Venn diagram showing the number of CGis that are differentially methylated.

FIG. 3A-FIG. 3C. Semi-supervised analyses between bladder tumor subgroups involving hierarchical clustering and PCA. (a) Clustering and PCA of NMI-mt and NMI-wt tumors, (b) Clustering and PCA of MI and NMI-wt tumors, (c) Clustering and PCA of NMI-mt and MI tumors.

FIG. 4A-FIG. 4B. Most methylated gene-associated CGis are targets of polycomb complexes. (a) Overrepresentation of PcG genes in BC methylated CGis. (b), Venn diagram showing the overlap in PcG target genes between bladder cancer subgroups.

FIG. 5A-FIG. 5D. A. Hierarchical clustering of GGMA data of all the investigated samples. B. Heat map showing the probes which are methylated in all tumor subgroups vs. blood and urine. C) Methylation of two CpGs in the MEISI CGI (black peaks: methylated CpG, red: not methylated. IM, in vitro methylated DNA; RTI 12, T24, J82: bladder cancer cell lines; PU: patient urine; NU: normal urine; NB: blood), D) KM curve for progression to muscle-invasive disease based on a combination of methylation of GRP103, DBC1 and GATA2.

TABLE 1
Coordinates of selected regions containing relevant CpG islands based
on Homo sapiens full genome as provided by UCSC (hg18, March 2006)

| | | |
|---|---|---|
| chr1: 119333484-119333719 | chr1: 149638545-149639227 | chr1: 160220156-160220709 |
| chr1: 178464743-178471598 | chr1: 2072175-2072389 | chr1: 2106293-2106715 |
| chr1: 232106869-232108183 | chr1: 33385564-33385979 | chr1: 41122328-41122880 |
| chr1: 47672249-47672972 | chr1: 68288825-68289104 | chr1: 71284766-71286392 |
| chr10: 21828640-21829645 | chr10: 22804715-22807056 | chr10: 73437163-73438385 |
| chr10: 7449476-7449790 | chr10: 7489383-7495345 | chr12: 113657930-113659205 |
| chr12: 118612357-118612674 | chr12: 94776044-94776377 | chr13: 113930978-113931584 |
| chr13: 34950488-34951119 | chr13: 49595986-49600287 | chr13: 94152191-94153185 |
| chr14: 36205192-36206099 | chr14: 37746906-37747538 | chr14: 53488428-53488700 |
| chr15: 94688798-94689131 | chr15: 94696311-94696840 | chr15: 94705727-94706054 |
| chr15: 94712480-94712812 | chr16: 52873104-52882105 | chr16: 52873104-52882105 |
| chr16: 85098883-85102729 | chr16: 86294154-86294408 | chr16: 87372445-87372729 |
| chr17: 24303411-24303889 | chr17: 34157398-34159854 | chr17: 44074280-44075233 |
| chr17: 56827843-56838048 | chr17: 58865328-58865618 | chr17: 73932992-73933361 |
| chr17: 75377960-75381951 | chr17: 75531280-75531546 | chr18: 5186244-5187389 |
| chr18: 68359955-68362770 | chr19: 4059891-4060207 | chr19: 42586165-42586705 |
| chr19: 63420090-63420541 | chr19: 63784504-63785085 | chr2: 104835284-104839920 |
| chr2: 105864220-105865009 | chr2: 111591678-111597436 | chr2: 115635091-115637285 |
| chr2: 124498611-124499725 | chr2: 156892636-156892878 | chr2: 156893736-156894685 |
| chr2: 171384729-171385226 | chr2: 176737610-176738187 | chr2: 19424445-19425131 |
| chr2: 43251514-43251780 | chr2: 45249374-45251726 | chr2: 63134470-63134933 |
| chr2: 66525936-66527222 | chr2: 70984709-70985764 | chr2: 72224630-72228512 |
| chr20: 22505518-22507240 | chr20: 22510737-22514165 | chr20: 22514762-22515148 |
| chr20: 35582018-35583550 | chr20: 54012011-54014085 | chr20: 60061693-60062033 |
| chr21: 33316999-33322115 | chr21: 36990008-36995832 | chr21: 37857725-37858416 |
| chr21: 43350621-43351134 | chr3: 129688131-129694023 | chr3: 148591199-148594390 |
| chr3: 62330280-62330608 | chr3: 62331767-62332398 | chr4: 111780013-111780343 |
| chr4: 122520930-122521740 | chr4: 155881084-155881434 | chr4: 177159356-177159814 |
| chr4: 40134579-40135662 | chr4: 9756136-9756429 | chr5: 115810975-115811393 |
| chr5: 122461778-122463450 | chr5: 134390896-134393045 | chr5: 135442969-135443206 |
| chr5: 140724146-140724826 | chr5: 174091287-174092335 | chr6: 101023927-101024343 |
| chr6: 1549606-1560865 | chr6: 21704622-21705099 | chr6: 26312699-26313302 |
| chr7: 121727243-121727884 | chr7: 1481682-1482268 | chr7: 155266364-155266696 |
| chr7: 155288454-155292175 | chr7: 156895132-156895886 | chr7: 2082782-2083345 |
| chr7: 27164708-27165039 | chr7: 27219187-27220360 | chr7: 2724511-2724848 |
| chr7: 27251165-27252762 | chr7: 32768010-32768497 | chr7: 55040457-55041024 |
| chr7: 8449587-8450236 | chr7: 96488158-96489591 | chr8: 132985505-132986242 |
| chr8: 142288380-142288718 | chr8: 57520625-57522095 | chr8: 65661156-65662687 |
| chr8: 9798161-9799053 | chr9: 121170908-121172141 | chrX: 39749470-39754050 |
| chrX: 50040955-50041296 | | |

TABLE 2
CpG islands differentially methylated in NMI-BC wt vs invasive BC.
Provided for each are: ID; Comparision; Coordinates of selected regions containing relevant
CpG islands based on Homo sapiens full genome as provided by UCSC (hg18, March 2006);
Chromosome; GeneName; Location relative to gene Sequence.

7; I vs SW; chr1: 41122328-41122880; 1; chr1: 041122606-041122650; Unknown
CGGATGTGCGTGGACTCTGCGGGCGGGGCGCCCGTAGTCTCCCTCCTTCGCACCCCGGGCGCCTGGGAACCTCCTC
GCGCGTTTGGCCACTCGCTGCTCCTGGCAGTAGGGGCTTCGGGCCCCGCCGTGTCCCCACCCAGGTATGGGGATGC
GGAGTTGATGGGTGCGGGGCGCTGGGAGGGGGCAGATGAAAGGGGCAGTCGAAAGGGGGCCATGCGCGCCGAGC
GCAGCCGGGTCATTAGCCGCGGGTGTGAGCGGCGGGGCAGGCTTATCGCCCATCCAGGCCTGGCGGGCGGGCGGT
CCCCGACGCGGCTGCCGCCGCCGCCCGCGGCAGAGGGCACGGCGGTTCCCACGCTCGGGCCCTGGTTCGGGGGCGT
TCTCGGGACGGCGTGGCCGCGCTCACCACCACCGCGGAGGCCGGGCTGTAATTAGGATAATTGCGCTGCTCCCAGC
CTGGGGCAGCTCAGGACCCGGCTGCACCGCGCTGGCCCGGGACGGGCGGGGGCAGGGGGTGGCGGGGCCCGAGC
GATACAGCCCCTGGCGCAGTCCG
61; I vs SW; chr10: 73437163-73438385; 10; CHST3; INSIDE
CGCCCAGACCGGCCGTGGCGGGGCCCCGGCGCCACGTGCTGCTCATGGCCACCACGCGCACCGGCTCCTCGTTCGT
GGGCGAGTTCTTCAACCAGCAGGGCAACATCTTCTACCTCTTCGAGCCGCTTGGCACATCGAGCGCACAGTGTCCTT
CGAGCCGGGGGGCGCCAACGCCGCGGGCTCGGCCCTGGTGTACCGCGACGTGCTCAAGCAGCTCTTCCTGTGCGAC
CTGTACGTGCTGGAGCACTTCATCACGCCGCTGCCCGAGGACCACCTGACTCAGTTCATGTTCCGCCGGGCTCCAGC
CGCTCCCTGTGCGAGGACCCCGTCTGTACGCCCTTCGTCAAGAAGGTCTTCGAGAAGTACCACTGCAAGAACCGCC
GCTGCGGCCCCCTCAACGTGACGCTGGCCGCAGAGGCCTGCCGCCGCAAGGAGCACATGGCCCTCAAGGCGGTGC
GCATCCGGCAGCTGAGTTCCTGCAGCCGCTGGCCGAGGACCCCCGCCTGGACCTGCGCGTCATCCAGCTGGTGCGC
GACCCCCGGGCCGTGCTGGCCTCGCGCATGGTGGCCTTCGCCGGCAAGTATAAGACCTGGAAGAAGTGGCTGGAC
GACGAGGGCCAGGACGGCCTGAGGGAAGAGGAGGTCAGCGGCTGCGGGGCAACTGCGAGAGCATCCGCCTGTCC
GCGGAGCTGGGGCTGCGGCAGCCCGCCTGGCTGCGGGGCCGCTACATGCTGGTGCGCTACGAGGACGTGGCACGC
GGGCCGCTGCAGAAGGCCCGCGAGATGTACCGCTTCGCCGGCATCCCCCTGACCCCGCGGTGGAAGACTGGATCCA
AAAGAACACGCAGGCGGCCCACGACGGCAGCGGCATCTACTCCACGCAGAAGAACTCCTCGGAGCAGTTCGAGAA
GTGGCGCTTCAGCATGCCCTTCAAGCTGGCCCAGGTGGTGCAGGCCGCCTGCGGCCCTGCCATGCGCCTCTTCGGCT
ACAACTGGCGCGGGACGCCGCCGCCCTCACCAACCGCTCAGTCAGCCTGCTGGAGGAGAGGGGCACCTTCTGGGTC
ACGTAGGGGGGCCGGGGCCCCGTATGCCCCTCCTCGTGAAAGGCCTGCCCCGTCTTTCTGCCGCAGCCCTCGCAGA
GGGCGGGTGCACAGCGCCATGACGGGCAGCGCCTCCTGTAGCAGTAGGGCCCCCAGCCAGCGCTCCAGCCAA
97; I vs SW; chr12: 118612357-118612674; 12; CIT; INSIDE
CGGGGTCCTCACGGCTCCCGCAGGCAGCCGGCCCCTGCTGCTGTCTTCAAACAGCCTCCCGGGGGACCGCTCTCTC
CGCGTGCTGAGCATCCGGCCGGGGGACTTCTCTCGCTCCAGGGGGCGGCCAGAGACTTGTCCCTGCGCAGCTCGGT
CCGCCCCTCGCGGTAGCGGTGGGGTGTGCTTGGCTCTCGCGGGTGGCTGGGGCCTTCGGGCGGCGCTGGGCTGGAG
GCCACGCGCTTGGTGATGTGCTCGTTGTACGTGGGTGGGCCTCGCTTGTTGGGGCTGCTGGCGACACAAGAGAACG
TAGGGAGCTGCG
162; I vs SW; chr17: 34157398-34159854; 17; PCGF2-PSMB3; DIVERGENT_PROMOTER
CGAGAGGACCCGGACCCCGGCGGGGAAGGGCGCCCTCCCCCGGCCTCGCGGCCGCCCCAAAGCACATCCCCTTCT
GCCGGCCCCAAGTCCCCGCGCGTCCACGGCGCCCCTGTGCCCACGGGAGAGCACCCCCGCCCAGATGGAAAAGGG
GGTAGACCGGGCAGAGCGAGAGGCGGCTTTGGCTCGTGGGGAGCGGCGACCCAACCCAGCGCGCATCCCGCAAGC
CCTCCCGGCTGCCCCGGACCTTCCCCCTCGCTACCTCGGAACAGGGTCTGCCCGGGGGCTGCTGCACAAAGAGGGG
TGGTCGAGGGAGACGCCAAATCGTTAAGACGGGGAGCCCGTTTCGGCCATCTTGGAAGAGAGGAAAGGGAAAAAG
GGACAGAAAAAAGGGAGAAAGAAAGGCGGACGGGGCAAGCTGGGCTAGGGACTGACGGGGCGCGGGGGGTGCG
AAGGCAGGATCGGCTCAGAGCCCCCCCTCCCATCGGGGCTTGCGAAGAACGGCCGGCCCCCATCCCAATCCCCAGA
GGGTTACAGGCCAGGAGAGGGAAAGGCAGGGGGAGTCCGCTCCGCTTACCTGGGTTCGGGGTCCGGTGGGTCTCG
GGGAGGGGGGGATGGGAGGGAGGGAGGGAAGGGAGGGGAGGGGGCCGCAGCCGTGTCGCTCGCCCGGGCGGCG
GGAGGGGAGAAACCTACGGTAAGAAAGGAGTTTGTGAAAGCGGCTTGGGGTGGGAGGGAGAGGGGAGGGGAGGG
GACCGAGGGGGGAGGGGAGGGACCGGAGTGTGGGGGGGAGACAAAATGGCTTTTTTCCTCCAGACAAGGTAGGTC
CCGCCCACCACTCACCCACGTGACCTCATTCCTTCAGGATGCTGGCAGAGACGGGAAGAGGAGGGGGAGGCTGGC
GCGGCCGCTCCCCGCCACCGGCTGCCGGGTCCCTAGCCAGGACGAGACCCCTCCCTTCCCATGCGGCCGCCCCGAC
CCCGGAGGATGGATCTCTTATGTTACCTTCCGCAGTTAGCCCCCCTCCCTTCAAAATATGGGTTCCCCCCACTGACA
CTGTGATGCCCATCCCTAACTGGGGCGCCTGGAAAATGGTGAAATGGGGACTGGGCTGAAAGGTGGCTCGTGGTCC
TCCACGCCCCGGCCTAGACCAAAGGGCGCGGGGTCCGGCGGGGGACGCGACGCCTTCAGCCAGGCGTCCGAGCGA
TTTTCATT
169; I vs SW; chr17: 56827843-56838048; 17; TBX2; INSIDE
CGGAGGCGGCACAGCTGGAGCCCGGATTGTGGCACGCCGTCACCGTGCTGCTCCGGGGAATCCCGACCCGCTCCCT
GCGAAAGCGTTTCCGAACGCGAACCCAGAGCCTGTGAACGCGCCGGCAAGCCCCCACTCCCCCACCGCCGCCCGTC
GCAGGTGGGCCCGTCCTAGGGGTCCTTCCTGCGCTCTACCCCGTCTCTCAAGTCACTCAGTCGATCGCCCCGTTCAC
GCTCCCGTGATCCCAGACATCCATAACCACGATCTCGCCTCCATGCACATCCAACGCACGACGGTGCACAACGTGC
ACCTGACTTCTGCGGACCAGGTGTCTCAAGCGTACAGCGGCCACCCGCGGAACCGCGGCCCGGGGCCAGTGAGTC
GTCTGCAGCTCCCCGGGTTAGGGGATTCCCCAGAACTCCGGGAAAGTCACCCGAAGTCCATCCGGGATGCGGCCTT
GGTTCTCGGCCGCGTATCTCGCCCTTAGGTGCAGAACGACGCCCTTCCAGGGCCCACAGCTGCCAGGCTGGGCCTT
GCCCTCGCATCCCCCGGGAAGACCAGGGACGGGGCCACACAGGCCGTGGCTGCGGAGACGCTTCCCCGGGCCACC
CCGCGACCAGGAGGGAGTCGAGCCGCCCGCTCTCCCGCGTCCCCCGCCCCATCCCAGCCAGTTGGCCCCACCCTGC
GCGGGATAATTGGGACGGGAGGGAGGCGACGGGAGGGCGGCGGCTCCAGAGAGACTGCGCGCCTGTCAGCGGCA
ATTTGTTTAAGTGGACGGGACGGGCCGGGCCGCTGCGGGCTGGGGTCACCGAGGCCGCGCCCCCACCCCAACCAG
ACCTGGGACCGCGGGGGAGCCCGGTCCGGCCGCTAAACCGGGCTGGCTGGCGCCAGGGCTCCGGGAGGTGCGGTC
CGGCGGGGAAGCCGTGATGGGAAGCGACTCTGTCCAGGGAGTGTCCTTCACCACCACACTCCTCACGTCCAGGCAG
TGATGACGGCCTGGCGGCACCCTCACAGCGGGCCCATAGCACGGGGCCACACACGTCCCCTGAGCTTAGCCTGGGC
ACATTCGTCTGCCACCGAGGGCTTAAGCCAGTCTGCAGCCCGCGCCCCGTCACTCGGACGCAAGTCCGTCGTCCGC
TCTGCCACGCGGCCGCACAGCCCGGCTTCCTGCTGCCCACTGCCCGCGGGGTCACGCAACCCCGGCCCTGCACA
270; I vs SW; chr21: 37857725-37858416; 21; chr21: 037857850-037857894; Unknown
CGATTTCCGCAGATTTCCTGCTGGAACGTTCTCTCGGGCCTCAAGTTTTCCTGAGAGGACGACGTGGAGAGTGGAG
ACAAAGATGCTCAAAAGCCAGGAAATCGCAGGCTCGGAAGCCCCCACGCATCCTCTGAACGCAGCGCCATCTCGG
GGCTGCGGCGGGACCAAGCGGGACGCTTGCAGGCTGTCGGCGGCGGGGGCCGCGAGCCGCCGGCGTAGGCCGCTC
CAGCTGGAAGACCCCACCGCTAGGGTTCCAGGGGGCTGCGGACGCTTCCGAGGGGCCCAAGGCGAAAGAACGCGC
CCGCTTGTCGGCCACGTGACCGTCCCCGTCCGCGTCCGCAGCGACCACGTGACTGTGCGTCCGCGGCGTCGATTGA
CTGGGAGCTCCCTGGCCACGCCCTAAGCGCCAGCCCCGGGCTTCGGGGGGGGTGCTGCTGCGGTTGCCATGGCGT
CTTCCGAGCGCGCAGGCGGGGCCCAGAGTAGCTTTTCGGGTATTTGCTACCGGCTCTCGGTAGGGCTGGTCACCTTT
GCTTGCTGAGCTTCGCGCCCTGCGTCCTTTGGTCCCCAGCAACTTGCAGGGCAGCCGTCCCTCATGAGGCCCTCCAG
CGAATGCCCTAAAGCTCCGAAGTTGCTCCGGAGGAAACAGGCTTGCTCCGAGGGTGCACGTCCTTACTGCCCGTGA
CGTCG
271; I vs SW; chr21: 43350621-43351134; 21; CBS; INSIDE
CTGCTGCACTTGTAAAGTGGGTGCTTCTCAGCTTCATGGTTTGAGGGGTGCAGCCAGCTTCGGCCTAAGTGGTGTTG
AGGGTCGAGGACCCAGCACCCCCACACGGGAAGCGCGGTGTCCCGCCTTCAGGCTGGAGTACTCTGGCACCCTCTG
GGGTCCCGAAGTGCCTGAGTACCTGTTTGAGCCGCGTGTAGGTGACTGCGCACCTGTTTGAACTGCGTGTAGGTGA
CCGCGCACCTGTTTGAGCTGCCTGTAGCTGACCGGGCACCTGTTTGAGCTGCCTGTAGGTGACCGCGCACTGTTTGA
GCTGCCTGTAGGTGACTGGGCACCCGTTTGAGCTGCCTGTAGGTGACCGCGCACCCGTTTGAGCTGCCTGTAGGTG
ACCGGGCACCCGTTTGAGCTGCCTGTAGGTGACCGGGCACCCGTTTGAGCTGCCTGTAGGTGACTGGGCACCCGTT
TGAGCTGCCTGTAGTGACCGCGCACCCGCTTGAGCTGCCTGTAGCTGACCGCG
283; I vs SW; chr4: 40134579-40135662; 4; FLJ20273; INSIDE
CGCGTTGTAGGGGTAGCCGTAGTAGGCCAGTGTGTAGGGGTCGCAGGAGTACACGTAGCTGGGCTGCTGCGCTGCC
TCAGCCGCGCCGCCGCCCCTGGCTGCCTTCTGGTAGCGCGAGTACTGCTCCTGTCCACGGGCTTGGCCAGCGTGACC
TCCAGGCACGAGCCCTCCAGCTCAGTGCCGTTGAGGTTGTTCATGGCATGCACGGCATCCTCGCGGCTGGTGAAGT
GCACGAAGGCGTAGTCGCGGATCTTCTTGACGCGCTCCACGCAGCCGGGGTTGAACTGGCCGAAGCTCTTTTGATG
GTGTCCTCGGTGGTCTCGATCATGAGGTTGCGCACGTAGAGGATCTTCACGGTCTCCATCACGTCCTCGTCCACGTC
GATCTCAGGTTCGGCCCAGTCCACGGCGATCTGGTGGCCCCACAGCTGGATGCGGCCAGGCATGAGCTTGCGGCGA
GCCATGGCAGCCGGCGGTGGCTCTCGTACTCCACGAAGGCGAAGCCGCGGTTCTTCATCTTGTCGGCCGCGCTGGC
GTAGACGATCACGTCCAGCACGCCCTCGGTGACCTTGGCAATCTCCTCCAGGATTTCCTCGCGCTTCTTCATCTTGG
GGATCCCGCCGATGAAGAGGCGGCAGTTGTCCCGCTGCAGCACACGCCGAGCAGGCGGCCCGGGCGGATCTCGTA
GTTGTTGAGCTCACGCACTGCGCGCTTGGCCTCGTGCTTGTGGCAGTACATGACGAAGGCGTAGCCGCGGTTCTTGC
CGTCAAAGTCCATCATGAGGCGCAGCTCGTAGATGCGGCCCACGGCCTCGAAACGGGCACCAGCTCGTCCTCGTAC
ACGTCGCGCGGGATCTTGCCCACGAAGACCTCGCAGCCACGCTGCGGGTGCGGGCCCTCCCAGCCGGGCGGTGGG
CCGCCGTACTTGCGCTGCCCGTTCTCTTGCACCATGCTGTAGCCCGTGCGCTCCATCAGCGCCAGCAGTGCTGCTCG
TTGGGCGCGCCCGCCACGCCCTCGGGCACCTTGGCGGAGGACCCGGCGGCCGAGTCACTGCTCATGGCTGCGGTGG
AATCCTCTGCG
357; I vs SW; chr7: 27219187-27220360; 7; chr7: 027219187-027219246; Unknown
AAGTCTGGAGATAATTATGTCGTACAGTCGCAAACATTATTCCGTTCTTACTGTAAACGGCCCCGGCCACCTTTACG
AGAAACCAGGAAACTTCTGAGAGTTACTAGCAGCGTTTACGCGGGCAAACGAGTTCTTTTTCTTTCTCTCCCGGATT
GTTCGAAGTATCTATCGGGCGGCTTCGATGCCAGGTTCAGAGGCGCGCCAGGGAGAGGGCGCCCCGCAGAGGAGC
GCAGCGGAGAGGCCTACGCAGGTCCCCGGTGCCCGCGGCCCTCGGAGGCCGGGCCCTGCGTCTTGGCCAGCACTG
GGTGGCAGCTGAGGCTGGTGGCCCGGAGCCCTCGCGGCCGCGGGCAGGCCCCTTCTTGGGCAGGGTCGGGCACTCC
CGCTGTCCAGGGCTCTTCGGCACCCTCCTTCCAATCAGGTCGCTCTCCCCTGCTCCCCAGACTCAACTCCTCCGAAG
CTGCTCCAGGTTGAATGTGACCGCTAGGCCGACTCCCTGGGCCCGCGAGCAGTTCTCGAAAGGTGCGGACTGAGCC
CTTTCTGGGGTGGGGTGCGGGTTGGTTCTCGCAAGTGTGACCCAGGGTGAACTTGCTATTTCGGGTCCCGGGTGCTG
CAGGGCCAGGAGAACAGCTGGGATGGGGGACCCCGCCTCCACCCTCGGGCCGGCACGTCCGCGCCCTGTCAGGTC
CCCCTCCCTCCTCTATGATGGCCAAGGCGTGCGCCAGGGCTATCCGGGAACCTTGTAAGGCCTCGTGCTGGCACCT
AACCCCACTCGCGGCACACTTCCTCTATGTAGTCTGCGGCCCCGCCTGCCAAATAGAGTGACCAGTGCAGGGACAG
AATGCCAGGCTGGTGGCCGACCGCCTGAGGGACAAAGGCGAGCATTCACAAGCCAACAGCAGACCCCTGCCCCCC
ATATTTCCATTTCGCTCAGGCTTTTAGGACAAAATCAACAAGGCCGCAGAGTGGTGCAGGCGCTCACCCCGGGTGC
AGCCTGGGGAGCCACTGGTTCCGCGACCCTGGGCATGAAACTCCTCAAGGGCGGCCCTCGAGACGCAGGGGAGAG
GATGCTGCCGGCGCCTGCCCGAGGGCTTCTCTGCGGGAAGCGGGCAGGCACCCCACCGGAGTCATTGCCGGGACCC
TCAGCGCAACGCGGGCCTGTTCCTCTCG
373; I vs SW; chr7: 156895132-156895886; 7; DNAJB6; INSIDE
CGGAGATGGCGCGTCTGGATTCAGGGAGATCGTGCGGCCAGCTTCCCTCCCTCTGCTCTGGATGGGGGCCGGCCGC
CGCCGCTCACTCACGGCTCTCTCTCTCCCGCTGTGCCTGCAGGTGTGGCCGCGACGATGCCCTCGCTGAGGAGCGCA
TGCGGAGAGGCCAGAACGCCCTGCCAGCCCAGCCTGCCGGCCTCCGCCCGCCGAAGCCGCCCCGGCCTGCCTCGCT
GCTGAGACACGCGCCTCACTGTCTCTCTGAGGAGGAGGGCGAGCAGGACCGACCTCGGGCACCCGGGCCCGGGAC
CCCCTCGCGTCCGCAGCAGGTGTGCAAAGGGAGGCAGCCGTGGAGCAGGCGCAGAGTGAGACTTCTCTGGGTGCC
AGAGGACAGCGAGGACACAAACCGCGCCTGGGCTGCTGTGTTCTGACCTGAGCGGGCGTGGGCGGTCGGGCGGGG
CGGGAGGAGGTAGCCTCCTCTGCTCCTCTCCCGCTCGCGGCGCCCCACGCCACGGTGGCTCCGGGCCCCCCACCGT
TAAAACGGGGTCCGCGTGTCCTGTACGCAGCGTGTTGCTCCGCAAAGGATGACAGAGCTGCCACGGCAGTGCGAG
GTGCTGCTTTTACCGTAGACGTGCCTTTTCGTAGCTTTAGCGTAGAGCTTGAGGCCTGTGGCTTAGGCCGGGTGGTC
TCGTGCTGGTGGGGTCTGCGTTTGCCCCTGCCCCTCCGTCAGCTTCACCTTCTCGTGCCTTCTTCTGCG
389; I vs SW; chr8: 132985505-132986242; 8; chr8: 132985506-132985550; Unknown
CGTAACGGGCGTGGGTCGTCCGTCGCGCCGCCCGGCGAGGAGTGGGCTGGCGGCGGTAGCTGTCGCCCGCTTGGTT
GCGTGACCGCGGGGTCCGCGTCCGCTCCCTCCACCCTTCGCCCTTCGCCCTCGCCTCGTTCCGGCCTCCGCGGCCCA
GCAACGGCCGTCATGGTGCCGTCGGCGCTCCCTGCGCGGCCCCGCTGAGCCTCGGTGCGGCGGCGAGCGCGGTCGA
GATCGCCATGCCTACCCGTGAGTGGCCGGCCGAGGGCCGGGGGCGTTGGGAGGCGACTGCCTGCGCGACGGCTTG
GGCCGGGTACTCCTCCCGGGCGGCTGGGGAGGCTGGGCCGCGCTGAGCAGAGCCAGGAAGTGTCTGCGAGCGGCG
GAGTTAGTTTCGTTTCGGCTTAGCCTTCCGGAGATGGCGTGAGCCCGTTGCACGGATCGGGAAACTGAGGCCCGAA
CGTTAAGGGAACTGCGGGACGCGCTGTTAGGAGGCGGCAGAGCTGGTGGTAACCTGACCCGTGGGATTTCAAAGT
TAAAGCCCTTTTCCCACGGCCACACGGCTTCGTGGGACTTTATGGAAATATTGAAAATAACTGCGGTACCATTGTCA
GTCCCTTCTCACCTCGGGCCGGACGTCGCTAGTGGAGCGCCGGCCCCGAGTCCCTTCCCGCGGCCGCCAGACAGAC
CCCCGACAGCCTAACTCGGCGTCCGTATCGCCGTCGGGGCGGCTGAAGTCG
12; SW vs I; chr1: 68288825-68289104; 1; DIRAS3; INSIDE
CGGAGGCACGAACCAAGCAGCCTAGAAGACAAATGCGCTGCTCGGAGAGACTGCCGCGGCAACCAACTGGACACC
CCAAGAGCTCACTCCTCCGCGGTTTTATATTCCGACTTGCGCACAGGAGCGGGTGCGGGGGCGCAGGGAGTGTGGG
TAACAGGCATAGATTCCGCTTGCGCAATACGTGGTAAGAAACCAGCTGTGAGGGGCTGGCCCAACGCAGAGCGGC
GCGAGGTCCGCTTGGCATCTACAACAATCGAGGGGCTAGTCCAGAAGTTCAAG
15; SW vs I; chr1: 71284766-71286392; 1; PTGER3; INSIDE
GCTCAGCTAGTAAGTGAGGAGCTCAGCTCTGAAGCTGTAACCAAGACCGCGCGGGCAGGAGGAAAGGACACTTCA
GCGCTCTGCGGTTGCAAACACACCCCTGCGGGTGCTGCCACTTAGCAAAGTCTAGGTTCCCGCTTACTCGCACACG
CATCCTGACTTCCCCCAACCCTGGAACTACCTACCAGGAGCGGAGACCAGCAGACCGACAGCACGCACATGATCCC
CATAAGCTGAATGGCCGTCTCGGTCGTGATGCGGCCCCACTGGGCACTGGACTGAGATGCCGTGGCCTTGGCCGGC
AGCGGGACACCAGGGCCTTAATGGTGGCCAGGTTGCAGGAAAAGGTGACTGTCAGCGCCAAGAGCCCCAGGAAGG
CAAAGGCAGAGGCGAAGAAAAGGTTGCCCCAGTTATGCGAAGAGCTAGTCCCGTTGCCCCCTCGCCCGGTGCTGAT
GAAGCACCACGTCCCGGCCACTGGACGGTGTACTGGCCCACGCCCAGCACCGGCAGCAGGGCGAAGGCGAGCACG
GCCAGCCACACGCCGAGCAGCACAGCGCGGGTGGCACGCGTCTTCATGTGGCTCGCATACCAGTGCGGCGCCCTGA
TGGCCAGCGCCCGCTCGACGGCCATGGCGCTGGCGATGACAACGAGGAGAGCCCGAAAACAGTCATGGTCAGCCC
GAAAAAGGTGCAGAGCCGCCCCGACGGGTCGATGTGCTCCCAACGCTGCTTGGACAGGTACACGACGATGACGAC
CGGGGTGGTGAGAAGCTGCCCGACCAGGTCGGTGAGCGCCAGCCAGCCGATGCACAGCAGAAGGACTTCTTGCGC
TTGCTCTCCCGGCGCCGGTAGCTGCGCGACACGAGCAGCATGGCCAGTGCGTTGCCCACGAAACCAGTGAGCAGCA
TGGTGATCGGGAAGGCCACGGACACCGATCCGCAATCCTCGCCAGACCCTGGAGGGCGCGTGAGGTTGCCCCGCG
CCTCGGGGAACGCTCGGGCGCCCACATGCCTGTGTAGGAGTGGTTGAGGCGGGTGCAGAAGGGGGCATCCCCTCC
GTAGCCCCGGGTCTCCTTCATGTTGGCTTCGAGGTGAGGAGGGGATGGCGTCCAGAGAGCCGCAGCGGGAGGGGG
CAGACGCGGCGCGGGCGGCGGCGGAGGTGGCGTTTACCGCGGCTGGGGCTGGGCTGCCCCCCATGGTGCGGGGCG
CAG
21; SW vs I; chr1: 119333484-119333719; 1; TBX15; INSIDE
ATTCTTAATTCCCACACCTCTTCCCAAGTTACGCCACCGGTCGAGGACGGCAGGAGACCCCCGAGTGCAGAGAAAG
CTCAAACCGGCAGCGAAGTCGGTCCTAGCCAAGCTGAAAAAACGTCTCGGATTCGCGGACAGCGGCCTAGACACA
GCCCGATCTTCCAGTCCTAGTGCCCTGGTCGAGACGGTTCTATCCTTTTGCAAAGAAGCCGGAAAGAGCTGGGTCC
CGGGGGCG
26; SW vs I; chr1: 178464743-178471598; 1; LHX4; INSIDE
CGGGGTGGGCTTCCCGCGCCTGCAGCCCGGAGGCGTCGAGCGCCTGATGGAGCGTCAGAGGAGACCTCGTCCCAG
CGTTGGGCCTTCTGTTGTATTTCTGCCAGAAACTCGCGTTGAGGAGAATTTGTTTAAGCGTTTCTCTACCTGTCCATG
CATCTCGTAAATACAGTAAGCATGGCAAGCCCTTTCGCTGGTTAACCCCGGCGTCCGTGTGCTCACTGCCGCCCTGC
ACGCACGAGGTCGCGGATGGTTCAAAGCACCCGCGGTGGCAGCCGGAGAGGCAGAGCGGGCTCTAGGGACTAAGA
CAGGGCCGAGCTGGACGACTGGTGGCAGGGCGGCCGGGGAGGCCCCGCCGCGGGGAGGTTTGGGCCCCCTACCCT
GCGGAACTGGAGGCAAGTGCGGACTCCGGCTGCAGGCCGCGGTCTGCGGGGACGCGCGCGGGGGGCCTGCGTCCC
GCCACCTCCGCGCACACCCCTGGTCCCCTTCCCCAGCCCGGGCCGGCGCCGCCCGCCGCGTGACTCGCGCGTTCCG
GGCCGAGTACGGGCTGTCCCTTGGCCCAGCCTCAGCCAGACGCGGGGGAGAGAGAGCAGGGGCTGGGAAGTGCCG
GCAGGAAAGGAATTGGCCAGGGGAAAGAGGAGGAGGAGATAGATGGAGTCATCGGTTACGGAGCGCGCTCCGTC
CGGCGGAGCGAAGCCGGGGACCGAGAGCGCGCAAGGCGAAGACACAGCGACGCAGCCCCGTCCCCGGCCTCCGG
ACCGCCTGCCCCGCTCGGCCCCGGCTCGGCTCCGCCCCGCCCCTGGGTCCGAACGGAGGAGGAGGAGGACACGAG
CCGCGCGGGCTTGGAACCCACGAAGACCCGCAAACCCAGGGCGCCGCCGTCTCCGACCACCTGCTGCCGCGGCAG
GCCATCAGCAGAATTTATACCCAGGGCGCGAGCCCGCCACGGCTGGGCGGAGGCGGAGGCGGAGGCGGGGAGGG
GGGGGGGGTCCGGCTTGCGAGGCCCCGCCCCGCTCCCTGCCGCCCCGCCCCCCTCCGCCCCGCCGGCCTCACTCGG
GGCGCCGGGACCCCCACTCCCTCCGGGGTCGGCTGCCCGCCGCCGGGTTGGGGGATGTGCCGAAGGGCGGGCCTCC
CTGCGGAAAGCGACGTCACGGCCGCTCCTCAGTGTGAATGAATCAAAACGCCCGGGTGACCGCGGCCTCCCGGGC
GGCC
40; SW vs I; chr10: 7449476-7449790; 10; SFMBT2; INSIDE
AAACCACTCTGAAAGAACTGTGAGACCTTTGCACTAAGACCATTAGGGATTCTACGCAAGGTTCTCTCTGCTCCTTG
CAAAATTACAGGCGTCATGAAGGATGGCGGCTCGTAAATCGAAGCCTTTGAACAGGGGCTCACCGTCCGGCGGCA
TCAACACCTTGTTGTTCTGTGTGCACCACCCCACGGGGTGCAAATCCGCGATGACTACGTCACACCAGAAGTCGGC
CCTGCGGTCCTCCCCGTAACCGCAGTAGCGCAGAAGCAGCAGCTGCCCGCACGTGGTAATGATCGTGGCCACCAGT
ACGTGTCCG
41; SW vs I; chr10: 7489383-7495345; 10; SFMBT2; INSIDE
CGGCAACCGAGCCAGATCCAAGAGGGGTCGGCCTCGTCTAAATTGGTTTCCCACCAATGGCCTCGGATCAGCCGCG
GCTGTGCTGCGGGAGCCCTCAGGACGCGGCTGGGGTTGGTGCGCGGGGCCCGGAACCCCAAACCCGGCTCGGTTC
GGCAAGGTTCAGGGAGACAAGGTAGAGAAGGCTGGGGTGAGCGAGAAGTCGGGCGGCCGATCGTCAGGGCCACG
AGCCTCGCCTTGCCTTCTTGGAATCCCACCCAACTTTAAAGGCCCAAAGATCCTGAAAATTCCGAAAGCGAAACGC
GAGCTGGTCTCCAGAAGTTTGAGAACGGTCTCCCAGGCTTTCCAGCGTCGTCCCGGGATTCTCGGACACCACAAAC
GCCATCAACCACGAGCACCGGTGTCCGTGGCTATTGCCCCGAATGGTCCCCATCCGCGTCCCCGGGAACTCCCTCG
GCTTTTCGCGCATCCAGGCCCCAGCCCCAGCTACTGGTGCGCCCCGAGCCCCTAGGTGCCAGAGCGGTGGTCGGCC
GGGCTCCTGCCCAGTCTCGGCTCCTCCCTCCTCCCCACCAGAAGGAAAAACTTGGGCCCTTCGAGAACCCTGTGGA
ATGTTCTTTGTAATCAACTGTACATCCGCTTCCACGGCCGGCCTCGTGCAAAATCGCGGGTTTCGGGGCCTTGGAGC
AAATTGCGCTTGTCAGCGGCGACGTCAGGAGGACAAGGGGAGGGGTTCGCGGCTGAAACTGCAGCTTCGCAGCAC
AGAGCCATTTTAGGCTGCTCCCCACCTCGCGGGGCCCATGGGAAAGCCGGCCCCGGGAGGCGCGCCCAAACGCAG
GCTGGAGGAGCCACGGACGCGTCCTGGCCGGCGTGTCGCCATCGTTCAGCCTCGCTGCCCAGGTGGGAGGGGTCAC
CTGCCGCGGGGTCTCCAAGCCAGTGCCGCTTGCTCCCGGCCCCCACCCACTGACAGCACGGCGTCCGAGTGACCCT
GTCAGCCTCGTTCTGCGCTCCTGCAAACCACGTTGCTGCGCTAACTACAAACCTGGCCAACATGTCTTTGTAACCCT
ATCATTTAAAAACGCTTCCAGGCACCTGGCCGCTGCCAGATCAGGTTCGCGGGCCCGGAGGAGGTCCTCCCACCTG
CCCCCGCCAGCCCCGGGGACCGGCGCGGCCTCCGTGTGGCCCCCGCCCACGAGGTCCCTCGGGCAGGAACCG
112; SW vs I; chr14: 37746906-37747538; 14; SSTR1; INSIDE
CGGAAAAACTCGAATGTCGCCCTGCCGTTCCTGGGGTTGGTGACAGGTCTGGTCATCGCACGGCGGCAGCTCCTCA
CCTGGATTTAGAAGAGCTGGCGTCCCCGCCCGCCCAAGCCTTTAAACTCTCTCTGCCAGAACCCGCCAACTCTCCAG
GGTACAAAGTACAGCAGGGACGCGGGTGGAGCCCTTCCAAGCGGCGCAGCCTTATCTTTCCCGAGTGAACACCTAG
GTGGATTCCCAACACCGCGCCTGGCACGTTTCTGGAGGGAGTCTCAAGCTCCTCCAGAGCTCCCAGCTGCCGTCCTC
GTTTCTGCAGTCGATATTCCTGTGGGAGACACGGGGGGCTCTGAGCGCTACGAGCTTTATTAAGAGATTTGCGAAT
GGTTCACTCAGGTCCCTGAACACTCCCAATAGCCTAAGCTGCCTGCTGTGTTATAGCGCAGAAGCCCCTAACGCAC
GGTGGTTGTCCTTCTTCTCATAACGCTCGCAGCTTAGGGCCAGTTTCCGCGATTCTAAGAGTAATTGCGTGGGCACC
TGTGCTGGGGCCAGGCGCAAAGAAGGGAGTTGGTCTGCGCGAAGATCGTCAACCTGCTAACAGACCGCACATGCA
CTTTGCACCGACCATCTACG
118; SW vs I; chr14: 53488428-53488700; 14; BMP4; INSIDE
CGGTAAAGATCCCGCATGTAGTCCGGAATGACGGCACTCTTGCTAGGCTGCGGGCGGCGGCGCAGCCCAAACATCT
GCAGAAGTGTCGCCTCGAAGTCCCGCAGGAGCTCATGGCTCTGCCCTGAGCGCGTCCTCCCGCGTGGCCCTGAATC
TCGGCGACTTTTTTCTTCCCCGTCTCAGGTATCAAACTAGCATGGCTCGCGCCTCCTAGCAGGACTTGGCATAATAA
AACGACCATCAGCATTCGGTTACCAGGAATCATGGTGTCTCTG
147; SW vs I; chr16: 52873104-52882105; 16; IRX3; PROMOTER
CGCGAGGATGGGCGGGCTCCACGTCTGGGGGCGCTGTGAGCAGGGCCAGAGCGAAACCTCAGTCTGAGTTTACGA
ACTGGGAAGGAGCCTGAACCCCAGAAACACCGTTAACAAAGAGGGCGAACGCAGGGAGCCGAGGAGACGTCTCC
TTGGCCTCTGGGGAAGGGGCTGGAGGAGCGAGCCAGCTGAGAAGCGGCTTTGCCGGGTGTCCACTAGCGGTTCGC
CAGCCCTCTGTGCGCCCGCAGAGTGACGCAGAGGCCGACACAGTCCCCTCCCCACTCGCCCCTGGGGCGCCGCCGA
CCTTGATCACCCCCTTCGCGGAGGGAACTCCCAAGGCGCGGCGCTGGCCGCTGGGTCCGCATGGCCCAGTCCTGTC
GGGCTCCGCTCTGCTGCCGCCGAGCTTCGAAGGTGTGCAGTGTTGAGCCGTGTCTTGTTCCGTGGCTTCGAGGGGTG
TGCGCGCTTCCTATACCTCCCGGAACGCGCGAGATAGTCACACGCGCCACTTTGAGGGTCAAACACCCCGCATCTG
GCCACACTGTACCTTTACTAACGTGGGGAGGGGGCAAAAAAATGTGTCTCTCCTTCTTACCCCGAGGTGTCACTCG
CCCAAACACTCCCTACAACTTCTTCAAGTCAAACTTGGCAAGGTTGGCTGGCTGACTGCGAGAGGAAAAAGAGGGC
GCGGAGGGGGCGCGGCGCGCGGCCGGGTGTAGAGGCCACGGAGGCGAGGCGCCGAGCGTCCCCTTTGTCCTGTAG
AGGGAGCTCCAGCCCCAAATTTCCCTGCTGCCTCCCCCGGCCCGCCCACCCCAGGCCCGCTGGAGCCGGAATCCCG
GCTGGAAAGGTGCGGCGGTCTGACACCCCCGCAACCCCCGCGCCGGGGCCTTAGCAGGATGCCTCTTCTAGGGCAC
GTGGGAAAACCCACCAGGGTGCCAAGACGCACAGATGCTCCCAGGACCCGGGCTCCCCAACTCTCAACTAGAGCT
GGGCGTCACACATCATCTAGAGGATTATGTACCCCTAGGCGACCCTCTTCTCCATATCCGCCTCCACCGTCACCCCA
CACCCTGGCATCTATAAAGAGGGAAGGGGGGCAACCGGGTTGGAAGGAGGATGGAATCTGGGCTTGCACAGCCTC
TTATAGGTAACAGTGAGAAAGCCTAACTGTGTCGATCAAAGAATGGGGACGAAGGGAGTGATGAGAACACGACC
151; SW vs I; chr16: 85098883-85102729; 16; FOXF1; PROMOTER
CACACACACACACACAAAAGTTGGGAGAGAATGTGAGAATGCTTGTGTTCGTGGACGCGTGCGCACGTATGGAGC
GGCGGTACCTATGATTATTTGCGCGTGGCGCCTGGCTATGCGTGGGCGAACGGCAGTGGAGAACATGTGGGCGTGC
ATGAAGTGTGCACTTAAACCAAAGTGTGCCCTCCCCGTTGGGCAGGCCTGTGTGTTGGTCCAAGGGTTGGGAGCGC
CTGAGCTGTGTGAGGGTCGCTGGGAAGGGTGTTTGAGGCCGTGGGCGGTGCGGAGCGTGTCTGTGTGCGCGGGGCT
GAGCTGGTTGTGTGAAGTGGTAGCGGGTCTGCCAGAGAGCGTTTACCTGCGTGAATGCACAACCACACACTACGGG
TCACGTCCCACACTCATTCTTTCCCTAGGCGGGCCAGGCTGTCTGCACGCGGAGACCCAGAGAGGCCAGCGGAAGG
GCAGCGCGGGAAGGCGCTCTCTTCTCCCGCGAGCGCAGATCTGGCGGAGGAGGCGGCGACTGCCCGGCAGGCTGC
GCCGGCGAGCCCCCCAGCTCCTCTCACCGACGTGCCAGGCGCCCCGAAGGCGACAGGCGTCTCCCGCCGAAAATG
AAGGCGTCCGAGCCAGGAAGGTGGGGGCGGGTAGTTAGGGGAGACAAGGAGTCCGGAGAAAACCGAGAGGCGGG
TAGGCTTTGAACGCGCCGCGCCGCGGGCCGTGCTGCGAGCCCGGCACACGCCTCTCGCCCCAGCTCGCGCTGGGGC
TTGGCCTCTAGGCTCTTCGCGATGACCCGCGAAAACACCCCTCGCCCCCCTCTTCTCAACCGGAACCCTGGAGGGA
GAGGCCGCGCCCGGCCAGCAGTCGCTGCGAGAGCAGCACTCACCTGGGTCTGTGCGCGCCGCGATGCGCCGGGGC
TCCTGCGCTGTCCTGCGCTCCGCTCTCGGCACCACCTTTCGTGGCTCCACCGCCCCCAGCCCGCACCCGAGCGCTGA
GGTGCAAGGGCAGGTCTGCGTGCGAGCCCCCTGCGGGCTGGCAGCCAGGTGCGCGAGGGCCGCCCTTGCCGGCGC
TCCCAGCCCGCGCTGTCTGCTCTCAGGAACCTGGCCTGGCGCGCAGTGTCAGAGGCCCCGCGGCGGCGGCAGGCCG
AGCCCACAGGGGCATTAGGCCAACTCCCCCTCCCGCCCTGCGCACGCGGAATTCTCTATTATTATTATTAAAGAA
180; SW vs I; chr17: 75377960-75381951; 17; CBX8; DOWNSTREAM
CGAGCAGGAGCAGGCGCGGCCCCGGATGTGGAGCCAGGCTTTCGGGAGAGCCCATTTCTACAGGGATTCGGCCCG
ACGGGTGCCGGGCCGAGCTCGCTCGGCCTTTGTTAGTTTCGGCGCCGCTGATTGATCGCCTAATCCAGTGGACCCTG
AGAAGAACAGACTGGGCTGCCAATGGCCCGACCCCACGCCGCCGCGACCTGCCCGAGGGCACCGGGAGCGATGGG
TGCGGAGGGGCTGGGAGGACGGTCATCCGGGCGGCTGGACCTCTCCTTCCTCCCCTCCGCCTTCGTGGCTCGGCGC
CGGGCCTCAGGGAGGACGCAGAGGGGGCGCCCGTGGCGGTGGGCGCCCAGCGGTACCTTTGGGTCGGGGGGAGGT
AAGGCTTTCTCAGCTCGGGTTCGGAACCAGAAAGCCACCGGCCCCCCGGGGGTAAGGGGTGGGCGCCGGCGACCC
GGGCCGACGCCGCCACCTCAGAGGGCGCGCGCGTCCCACCAAGGTCGGCCGCCGGCCCTTCGGACGGACGAGGGA
AAAGCCAGCCGCGGCTCCAGGGTGCCCGAGACTCAATCGCACCACCTGCACCCCGGCCACCTCCGCCTCCGAAGTC
TCAGGAGGAAAGCCCCGACCCAGCCCGCAGCCTCTCTCGCGCGTTCAGGACCCGAGGCTGCGGGCAGCCGCCTTCC
ACGACGGACAGCACGGAGTCCCGCCGGGCCCCGAGCCACGCCAGTCTCGGACTTCAGCTGGGTCCGAGGCTCCTCT
GGGCCTGGAGGGCTTTTAGAGGAGAAGAGGAAACCAGCACAGGAGCCCCAGAGGGCCGGACAGGTCTGTGCGCC
AGCGGCCCGGGCGCATGGCACAGTGCCTGGATCAGGATTGGTGCGAAGCCAGCATCAAACGGACAGAAAATGAAT
CCGATCCGACACTCTAGGAAGAGCATGAACCTTCCAATCCAACCAATGTTTGTGCGAGGCCAGGTTCCCCGCCGCG
GTGGCCCACAAGCCAGCCCTGTCCTCGGGACGCAAGGTAACCAGTCCGGCTGGAGGCCGCCACGCACGCCTGGTCT
CTGCAGTTGTGGCGGATTCCCAGGGACTCTGCCCAGGAAATGGAAACCCGTGAGGGCGCTCCTCAAAGCCGAGGG
TGGTGATGGCCAGCGAACCCGAAAAAGGGCAGGGTGGGCGCCCTCCCATCCGCTTAAGCAGTAGCCCATTCCACTG
C
187; SW vs I; chr18: 68359955-68362770; 18; CBLN2; INSIDE
CGCGCGCGCAGGTCCCCGCCCCTCTCCCCGGGCTGGGGGCGGGGTGGGCAGGCCGACGGCTGACCTGGTCGAAAT
AGATGGTCATGGTGCGGTTGCTCATCTCGGACGGCTCGTGGTTGGTGCTCCGGTGGCGGAGAAGGCCACCTTGGCG
CTGCCGGAGCGCACGGAGATGCCTAGGGAGGAGGTGACGGCGCCGTCCGCCGACGGGCTGGAGTCGCACACCACC
AGGCACTTGCCCTCCAGCACGATGGGCTCCGTGTCGTTCTGCGCCCGCACGGGGCAGCAGGCGGGCAGTAGCACAA
CAGCAGGGCCAGCGCCACCCCCAGGCAGGATCCGCAGCCGCCCGGCTCGCGCAGCGCCCCCCGGCGCCCGGGCAT
CATCAGCCGCAGCCCGAGTGGCCCCCGGCCGGGCGCCTGCATCGGGACTGGTGGGAGGCGGCGCGCGGGGGTGGA
GGCCGGCGCCGGCGCGAGCGCGCGGAAGGGCGCGAAGGAACGCGCGGAGCTCGCAGCAGCCTCCGGGGGCCTTC
GTCCCCGGCTCTGACGTTCAAGGCCAGGGTCGTTCTCAGAAGAAAGGCGCCTGTGAACCTGTCAGGGCACAGAACC
CAAAGCCTTACACCGGGAGGTGGAGCCACCGGGAAGAAGGGGACTGGGAGCAGGTGCCTGCGGCTCGGGGGTTCT
CCCCGAAGGCCACCCCCTTGGTCCCGCGCACAGCCAACCCGCTTTCCGATCTGGCACAACAGGTCCGGAGAAACGC
CCGGGCGCAGCTGGCAGCGCATCCCTGAAGGCTCCCGAGCAGCGCGCGGGGCTGCGACGAGGAGGGGGCCGCGCT
TCTGCAGGTCCTCGTCGCTAAGACGGGCCCCGAGACCCTCAGAAGGCGTCACCGCCACCCCGCCCATTCACCAGCG
CGGAGGGGGCTAAGACGCAGGCAAAGAAAAACCTACGAATGCTTGCCTTCTCGCCTTCATCTCACAGCATTTCCTT
GGAGCAGACCAAGGAACCCTTTTCCTATCCCTTGTTTCCCTGCTATCCTCTATTTTTCAATCCTCGAGATGTTTTTAA
AGTCCATTTAAGTCCCTTTGGTACAGATTTTATCTGTTTTCCACCCACAGAACAAATCCCAGGAATCTCCTTGGGGC
GAATAAGATTACTAGGGGGGAAATACACCACACACACACACACACACACACACACACACACACACACACACACA
200; SW vs I; chr19: 42586165-42586705; 19; ZNF569; DOWNSTREAM
GGAAGTATCTTGGCCTGCACGCGGTTTACATCATTGGGAGCACAGGCCTCGCCCTCCGAACTACGAGGCCCAGAGC
CCTTCGCGCCGAGCGTTTCCCGCCTTTCCCCTGTCTCAAGTCATTGCCCAGGGCCACTGCGGGCCGTTCCTGCCTGT
CCGTGGAGCTGCGGCCCCCGTGTTCCCAGGGTGAACGAGCACGTGCGAGGCGGTGGTGCTTGTATCCGGGATAACC
AGGTAGGGCGGCTGCATGCTTCATAGAGGCCAGCCTGTGCCGACCGGGAGCTGTAGGACGGTCTGTGTCTGGGCTG
CTTATGGGTATTCGCGTGCGTCTGCCGGTGCGCGGTCCGAGCCTACTCACGAGAGCGTGTGTGTGTCTTCTGTCTCG
TGTTGCTATGAGGTTTGCATCTGTGTGGCTGGAATAGCTTGTTTGTGGGGGCCCGCGCGTGACCTGTGTGTGCGTTA
CTGTGTGTGTCTAGGCAGGATAGTGACGGGCCGTGTGACTGTGACGCCATCATCAGGTTGGTGAGATGGCGTGACC
GCG
207; SW vs I; chr19: 63420090-63420541; 19; ZNF274; DOWNSTREAM
CGAGGGAGGACGACAAGCGGGGACGTTGGCGGAGGGGTTAATGGCGGAGTCGCGATCCAGCCGAGCCCACAACTC
TGGGAGGCTGGTTCTGCCTCTCTCCCGAAGCGTCGCCCGGCTAAGCTTCGAGACCAGGACACACAACCGCCGACTA
TTCCAGAGCCCACAGTCGGCTTCACGCTGGAGGCCGGAAACGACACGTCCCCAAAGCCCGCGCGCGCGGCCCGCC
AGAACTGACCTACCCAATCACCGCACCGCTGCGATCAAGCGGCGACACACGTTGCCGGGTAAGAGTCACGCTTGGC
AAATAAGCAACCTGCCTCATCCAATCAGCGTCTCGCCTGCCCGGTTAGTTCCTTGTCTTGCCCAACCAGATTCTTGC
CTCCTCCATACCGCTACTTGCGTGTGCCGGTGCCTGGCCCAATCAGCGTCTTGCGCCTCTTCTCGCTCCCG
223; SW vs I; chr2: 72224630-72228512; 2; CYP26B1; INSIDE
CGCTTGTTGCGGTGGATGTCGCCAATGGAATTGGACACCGTGTTGGGGCCCAGCAACATGCGGGTGCTGCGAGGCC
ACTCGGTGCTCACGAGGTGGTGCTCGCCCATGAGGATCTTGCGCACGTTCTCGCGCCGGTCACGCGTATCAGCGGC
CGCCCCAACAAATGCGTCTTGAACACGTTGCCATACTTCTCCCTCCGCGACGACTGGAAGCCAGAACCCTGCGGGA
GCCACACCCGGGTCTCTCTCAGGGTGCACTTCTGCAGAGGGCCCGCGAGGGAGGGGCGGCGGACCCCAACCGGGG
TACAGTCACCTGCCCCCTCTCCCCACCAAACACACTCACACAGACGAGCACGCAGGTTTTATTTTTCATAGCGTGCA
CAAGAACTGCGAAGTAGGGCCTAGAGGATAATAAATAATGCAAGGAGGCGGAGGCCCAGGGGCACCCCCAGGAG
GCTGTTTTTTGGTAATGCTTTCGAGAGGAAAGAGGTATCCCGGACAGCTGGACCCGAAGCGGGAGTCTCCGCCCCC
ACCCCCACCCCCACACCCCCACCCCGCGCTCGGGAGCCTCTCGGAATAAATATTTCCAGGCTCCGGGCCACGGGCT
GGCGAGACCCCGCGGGGTGGCCGCAGGCCAAAGATTATTATAGCGGTAAGCGGTCGGTGCCCGGAGGCAGCAACT
ACAGATGGGGGTTGGAGTTTCCTCCGCTCTCCGGTGTGGCCCGCGCAGGGACCCGGCGGCCCTCGGAGGACTCTGC
AGGGCGGGGTCACGGGCCCAGAGCCGCGATTTTCCTAATGCATTTTGCCCTGGAAATACGGAGACGGACTTTGGTT
GCCGCCCTGGAGTTTGGAACCCGAGCGCGGGGGGGGCGGGACCGGGCAGGGCGACCCGCGCAGGTAACCAGATC
CCCGGTGGTGGCGGCGCAAGCCGAGGATGGCGGGGCCGGGGCCGGCCGGACTCTCGAAACGGCTGGCGGAGCCGG
CCGGCGCGGGTACCGGAAACCGTGGAGACCTCAGAGGGGCGGGGGCAAGGGCGTCCCGCTCACCTTTGACGTCGG
CACTAGTTACCCCACCCCACGTTAACCCTTCTCCTGCCGGGGCAGCACCCGGCGCAAAGGGGAGGGGCGCCACCTG
TCTAGCCGCCCCGCCTGCCAGCCCAGAGCGTACCGACTCGGGCTCCAGATCTACCCGCTCCTGCACCCCCGAGGG
227; SW vs I; chr2: 104835284-104839920; 2; POU3F3; PROMOTER
CGCTCGGCAGAGGCACGATACAGCGGGAGAGAAGGGCAGGCCCGTTCACATTTTAATCGGAGCGCACCGGCGGCC
GCTCCTCGGCTGCGTCCTGGGCTGCCGCTCGGGCTCGGGACTGCCAGATGCAGCTCTGGCTGGGGGCGGCGGGCGC
AAGCGGGCGCACCCGCAGCTAGGGGTGCGGGGTGCACGCACACGCACGCTCATTAAGAGCCATGTATTTATTGAAT
GTCCGAGTTGGGTTAGTTCATTGGAAATCCCCGAGGAGGGCTCAATTTGCCCTTGTTTTCGTTGCCACTTTCCTTTTT
TCTTGGTTCGCTGAGGTTCCTCTGTGCAGCGTTTCCGCTTGGCCGCGTCCCCCCACCCCACCCCACCCCACCCCCGCT
TCTCTCGCCTACCGGGTGCACTCCCCCTCCCATCCCCCTTAACTCTTTCAGCTGGGTTAGAGCTGAGAAAGCATTTG
TCGCCGCCAGCCCATCCACCACGCAAATCCATCTGAGACAGAAAGGAAAGAAAAAAAAGCACCACCATGCCTAAG
AATAGAGAGCGAGCAAACCCCCCCACCGCTAATCACACACACACACACACACACACACACACACACACACGAGGA
AGCGGTGGAGCAGAGAAGGGCGCGGCTAGCCGACCCGGTTCTTTCGCCCGGCTCCTGCTGCCACAGGGAATTCCTA
AAGCCATTGGGGTCGAATACACTTACGATGAATCTATGGGGGAAGGTCGGACTGATTGCTTTTCAAATACATCGCA
CGGCTCCGCTGACCGGCACCCTCCAAACTCACAAGGGCACGCACGCTACTTGCCGAATCCCAGAGGAGGGAGGAG
GGAGGAAGGGAGGGAGAGCGAAGGAGGGAGAGAGGGGGTGGAGGAGCCAGGGAGCGGCGGCAGCGAGCGGTCC
GTCTCGCACGCGCGGGCACCGCGCTGGTCCTGGGCTGCAGGTTTCCCAGATGATGGCATCCGAGAACTTAAACAAA
GGGGCTGCCGCCGGCGCGCAACGGCTGCGGAAAGTTGCGGTGGCGGATTTCCAAGGAGCGTGGCCACGACCAGAG
CTCTTGGCGATGCGAGCCCCCGCTTCCCACCCCCGCCGATCAGAGAAGGGGGCCGGCTGGTGAAGGGAAGAGGAA
ACTTTGAAACCACTGGGGACACACTGTCTATAGGTATTAGCTTGAATGGTACATCCGTGCCGCGCGCTTTACA
235; SW vs I; chr2: 124498611-124499725; 2; CNTNAP5; PROMOTER
GACTCTCATCACTACCACACCCATGTGTCTTTCCTCTCTCCGTCTCCCTGAGACGCCAGTGAACCACCTGGGCTTGG
AATCCAGAGCCCCGGAACACCTTGCAGTACTTGCCCGAAGCAACGCGCCACAGCGGGAGGGAGACGCAGACGCGA
AGACCGGCGAGAGGATGTGCGGTGGGAGTAGCTGCGGGGTCTGCAAGCAGGGGAGCGCGCACAACGTTAAACCTT
GAAACCAGAAATCCCGATCCTATCGAGACCCATCAGCTTTTCCCATTTGGGAACGAGCGCAGGGGAAATTAATGCG
TAATTCAAGCTCTGTTTTCCCCTCCCCTCCAAAGGTGAGGTGGGGGAACGCGTCTCGTTAGCTCATTACAAAGCTTT
CCGTGGGAAATTCCCTCTTCCTAATGCGTCTGTCTTGCAACTCCTATTAGCATCCTTCGACAGCGGCCTTTGAAAAT
CTCCTTTAAATTTCATACCTCTACTCGTTTCCCAAATGGGGGGAAGGGAAGAGAAAGCCGGGGGGTAGAGAGAATT
TCCTTCCCCTTTCCCTCCCAGGTAGCCAAAGCGCACCGCAGCGGCTTGGCTTCCTGGAGGAAAAGCGCCCGAGCGG
GGCAAAGACCCGCCCAGGCTGCTCTTATAGGCGACGCGGGGGGGGCCTGGCTGGGGGCGGTGCCGCGCGGCCCCG
CTCGCCTATAAGGAGCTGTCCGCCACCCGGGTGCTGATTCCAGCTCTCGCGCCCGACGAGGTGGATTTGGCTGTCC
ACCGAGCTCCGGCGCCTGTCGTTCTAATTGGGTTTGGATTTGCACCGTTAAGGAGGGGGAAGAGAAGGAAGAGGC
GGGCGAGGAAGGCGAGTCCAGCTAGCGGCTGTTGCGGGGACCGTAGCCCCAGCTGCAGCTCCGAAGAATCCCCCG
CCACGGTTTCGGTGGAGCGTCTGGGCACGGGATGGAGTGAAAGAGCGAGTGCCTCTCCAAGCGGGGGTGGGAGGG
GGCAGGCTGTGCAGAGGAGAGAGACAGCGAGAAGAAGCCGCGGCTGGCTACTGCGAATTTGGGATTCGATTGGGA
GGGACCGCTCACTCGGGGGAAATGGATTCTTTACCACGGCTGACCAGCG
257; SW vs I; chr20: 22505518-22507240; 20; FOXA2/C20orf56; DOWNSTREAM
CGTACCCGGCTCTCAAAGGCACGGCACCTTTGAATTAGGTTGCGGAGTGGCCTGGGCGAGGGGACCCGGGGCCATC
AAAGGCCCGAGGGAAGGCCGTGGGGCGACACCCGTCTAATGACAAGTAATTAAGGATTCGACGCCGCGCATTACC
AGCGGAACGGGAGGCATTAATTATTTAAAAACGATTAGCCTTGCCGTGTGTCCTGCAGGCGTCCCATCATGTTCAG
CGAAGATAACCAGCCACCCGAGGCGAGCCGGGGCTCGGAGGGCGGCGGGCGCGCTCCCCAGCAGGCTTCGGGGCG
GTGGACACGGGCGCACAGCCCAAGGCACCAGGGAAGGAGAGGAGGGGCCACCAAGGGTCCCCACACAGGAAGGT
GACTACATAACACTGACCCTGCTGAACAACGAAACAGATTTCTGCCCACTTGGGTTCGAAGTCTGGTGACTGCGGC
GCAGATGGGAAAAGCTTTCGGTCGGGGGTGGTGGGCAATTTCCATGTGCCAGTTCCCTGTCGCATCAGACCAAAGA
CCCAAAGTAACCCAGCCAGGCACGGGCCTCTCACAGTGCCTGCTACCCAGGCGTTTAAAGAAAAGACCCCAGAGA
GAATGTGGATCTGGCGTGGGGGCAGGGATTGGGGGGGGGCCTGGTAGACACCTGGGGAAATACAACTTTATGAGG
TTTCAAGGGGCAGCAGCAGAACTACTATTTTTCCGAGGTCCACGCCTTTCCGCAGGCGGCGGAAAGACGCGGCTGG
TTCTGTTGGGTCCTACAAACTCGCGTCCTCCGGAATTCCCGAGGGCAAGCCCGGCACGCGCAGCGTGCTCCTGCCC
GTGGAAGGGGGGAAATCGGCGACTGGGTTGCAGGGAGAATGCGATGATAAATGGCCCTGCGTCTGCTTTCTGACTT
TCTCTCCTGGACCCAGCTCGTAACCCCAGAGGCTCCCCAACCTGCCTGCCGCCAAGCACGGCTGTGCGCAAAAGGC
CACCACCGCTTCCTGCAGCCAGAGCTAAGGTTCACTCGCCTCTTAGAAAGGCGCGGCCCCTCCCCTGCTGCTGCTTG
ATGACGATCCCCATCGTCTCCTCGGCCTGCCTGTGTCCGAGAAAGAGTTGAGACTGGACTCGAACCCCTGCGCGCC
AAGGGGAGGGCGTCACCATGCTCCGCCTCAGCCTCTTCAAACTCCCATGCACCACGCACATCATTTTCTGCTGA
262; SW vs I; chr20: 54012011-54014085; 20; CBLN4; INSIDE
CGACGTCCAGGGACCAGGTGCCCTTCGGCTCCCGCCGACCTGTTGCTCGAAACTTGCCCTGAGCTCTCGCTGCCGG
GCTCTGGGCTCCCAAGCCTCTCCCCCGCCGCCGCAGCAGCCTCTTTTAGGGCCCGGAAGAAATGGGAGCCGGGGCT
GGTGAGAGGGGTAGGAAGAGGGACGGTAGAAGTTTCAGACCCAGGCATATTTGGGAAGGCGAGTGCTTTACATGA
TTCCCCATTTCCCAATCGGACCAGCTTAGCCTGGGCAGGCAGCCTCGTGCTGAGGTGCCTGGAGACCGCCCACCCC
TAGGTGCTCGCTTCCCCCCGGGTCTGACCTGATCGAAGTAAATGATGCGCGTCTTGTTGCTCATCTCGGATGGCTCG
TGGTTGGTGCTCCGCACCGCCGAGAAGGCGACCTTGGAGTTGGCCGCCCGGACCGATATCCCCAGCGGGGAGGAA
GAGGAGCCCTTGGAGTCGTGGCCGGGTTCGAGTCGCACACCACCAGACACTTGCCCTCCAGCACGATGGGCTCCGT
GTCGTTCTGTGCCCAGACGGGCAGCCCCGGCAGCGTGAGGACCAGCAGCACGGCCGGCACCGCGGACAGCGCCCG
GCGCCCGGAGCCCATGGTGAGCCGTGTGGGCAGCCGCGCCGGCTGGCGCTGGTGCTCGCCCGCGTCGCCTCCTACC
CCGGGATCCCGGTGCTCGGGAAGATGCTAGCGGCTAGGTCGACAGCGCTGCAGGAGCGACGGCGGCGGCGGCGCG
CACACTTCCACCAATTCTGTGGCTTGAAGTCAAAGTCTCCCCTCGAGCTCTCTCGCTGCTCTGTTACCTTTGTCCTTT
AAGGAGCTCATGCAGCACCCTTTACCCTACTCTCCTCCGCCCAAGAATCAGCCCTGCCTGGGGCCCCTGCACCCACT
CTGGTTCCTAGACATCTGAAAGTCATCAAACCCTCACATTCACACCTCAAGGCAAAAAATAATAATAATAATAAAT
CTCACCCCAAACTCAAGCACCACCAGCTAAACCACGGAGCAGGAACAAAAAGAGGGGACTCAAAGAGAAGCCAC
AAGGGTGGCGGGTGCCCAGCGGCGCGGGTGCCAGTCCTGTCTGGCTTGCGGCAGGGACGAGTTACAGAGGCAGAA
GGTCCTTCCCAGGCTGAGAAGACGCGAGGCTGTGTTCATGGCCAGGACGCCAGCGACTCCCACTTTCGCCTG
268; SW vs I; chr21: 33316999-33322115; 21; OLIG2; PROMOTER
CGGTGCCGGGAGCCTTGCCTCCCGCCGCCACCCCTGGTCAGCTCTGCGCAAGAACGTCGTTCTGTTTGGCAGCCAG
GCCGAGACGCAGCCTGAATGTGAGCAGGAACTCGGAGAAGGGAAGGGAGAGATCAGAAAGAAGGCCCGGGAGGG
ACCCGGGAAGCAGTGGGAGGTCTGCGCCCTGGAGCCCCGCGAGAGCCCGCCGGTTTGGCACGGGCTCCTCCCGGG
CCGCCCGGCGGTCCAACAAAGGCCGGCCCCGACACGCACCCGGTCTTTTGTGGGAGAGAAACACAAAGAAGAGGA
AAAACACGGAGGAGGCCAACAGCACCAGGACGCGGGGGCCAACCAGGAACTCCCGGAGCCGGGGCCCATTAGCC
TCTGCAAATGAGCACTCCATTCCCCAGGAAGGGGCCCCAGCTGCGCGCGCTGGTGGGAACCGCAGTGCCTGGGACC
CGCCCAGGTCGCCCACCCCGGCGCCGGGCGCAGGACCCGGACAAGTCCTGGGGACGCCTCCAGGACGCACCAGGG
CAAGCTTGGGCACCGGGATCTAATTTCTAGTTATTCCTGGGACGGGGTGGGGAGGCATAGGAGACACACCGAGAG
GTACTCAGCATCCGATTGGCACCAGGGCCAAGGGAGCCCAGGGCGACACAGACCTCCCCGACCTCCCAAGCTACTC
CGGCGACGGGAGGATGTTGAGGGAAGCCTGCCAGGTGAAGAAGGGGCCAGCAGCAGCACAGAGCTTCCGACTTTG
CCTTCCAGGCTCTAGACTCGCGCCATGCCAAGACGGGCCCCTCGACTTTCACCCCTGACTCCCACTCCAGCCACTGG
ACCGAGCGCGCAAAGAACCTGAGACCGCTTGCTCTCACCGCCGCAAGTCGGTCGCAGGACAGACACCAGTGGGCA
GCAACAAAAAAAGAAACCGGGTTCCGGGACACGTGCCGGCGGCTGGACTAACCTCAGCGGCTGCAACCAAGGAGC
GCGCACGTTCGCCTGCTGGTGTTTATTAGCTACACTGGCAGGCGCACAACTCCGCGCCCCGACTGGTGGCCCCACA
GCGCGCACCACACATGGCCTCGCTGCTGTTGGCGGGGTAGGCCCGAAGGAGGCATCTACAAATGCCCGAGCCCTTT
CTGATCCCCACCCCCCCGCTCCCTGCGTCTCCGAGTGACAGATTCTACTAATTGAACGGTTATGGGTCATCCTTGTA
AC
276; SW vs I; chr3: 148591199-148594390; 3; ZIC4; INSIDE
GGCCGGCCGGCCTTCCTTCCTTCCGCCCTCGCCCTCTTCATGCCTCAGAAACGTGGCCTACTCTGCATTCGGTGTGT
GCGGAAGCAGCAATCACAGAGGCAGCCCTAATACCGGAGGCGGCGGCGGCGCAGCAGGGCCAGGTGGTAGCTCG
GGGCTGAGGATCGCGGCGGGGGCAGCCGCTATGGGGCCCAAGCCCTGACACACGTACCATTCGCTCAAGTCGGCG
GTACGCGCCGCCACCGCCGCCGAGGAGGCCACCTGGGACTTGTGGCCGCAGTCCGACGAGGGCGACACGAGGGAG
ACGGTGTAGCCGAATCGTAGCCAGAGCTGGGCGGCGGCGAGCGCCCGTGCACCTTCATGTGCTTACGCAGCGAGCT
GGGGTGCGTGTAGCACTTGTCGCAGCCCCGCACCTTGCACGTGTATGGCTTGTCGCTAGTGTGCACGTGCGAATGCT
TCTTACGGTCGCTGCTGTGGCGAAGCGCCGCTCGCAGCCCTCGAACTCGCATCTGAAGGGCTTCTCGCCTGGCGGA
GGCAACGCAGAGACATTAGTGCTTGTGGGTCGTGTTCCCGTCAGGTGCTTGCCACCCTCCCCCATTCGTCTCTCATT
TTCTGGAAAAGAACTACAAAATATTTTCAGAAATCCTTTCCACGGCGCCTCAGGTCGAGCACCCCTTTCCCTCGTGC
AGAGAGCGCCCCCGGTGCCCTCTCTTGAACGCCTCCATCCCTCCCGCCTTCCTCCTCTGGGCTCATGGGGAGGGTAT
GGAGGAGGAGCGACAGTGACTCCATCTTAGTCGAGTTTCCATCCTCGAAAATCCGATCCACTCGGGTTGTTTCCTCC
AAATTTTCTCCACTTGGAACCAGAAGCACCTCTGCTCGGAAATACATTAACGGAGGAGCTCACAATATAGTTAACG
GGGAAGCTCACATCTGCTCGATTTAAAGTTGCTGTTTCAGACTAACTTCTCTGCCGCTACCCCGCCCAGCCGTCATC
CCCCCCACCCACCCCCATCCTGGCCCAAATTGTTTCCTAAAGTAGGTTTTGCGCAAACGCCAAAGCGATGAAATAA
TTTAAGGATGCGCAGCCGATGCACATTGTGTGTGCATAAAGTGGATTCGTGCTGCAGGGAGAGGTATTCTGAGCAA
TGATTCACTTCAGAAGGATTTTTACAGGAATGGAGCCCCCTCCCTCTTTCCTTCTACCCCCTGAGG
285; SW vs I; chr4: 122520930-122521740; 4; GPR103; INSIDE
GAAATGTTCTGGAGCATGGTGACGGGAATGCAGAAGAAGGTGATGAGCAGGTCACTGAGCGCCAAGGAGCAGATA
AAGATGTTGGTGACGGTGCGCATGGCCTTGCTGCGGGTCACCACGTAGAACACAGAGCATTGCCAAAGAGCGCCA
GGGCGAAGATGAGCACGCCGGTGAGCACGAGGGCCAGCTTGGCGCGTCCCGGCAGCTCTGGGGTGTAGACGAGCG
GTCGCAGCCGGTACAGAGCGATGAACTGCTCCCGCGTCAGGTTGTGGTCCCGCAGCAGCCGAGAGAACTGCTCCGG
GTAATGTTAAGCGCCTGCATTGCTGTGCGCTCCCGGGACGCGGGGCCACCGCCCGCTACTGGCTGGCCATCCGCAT
CTGCGGGGCAGCGAGGGCTTCGGGGGACCAGCCGGAGGCCGCCTCCCTTCCTCTACTCTGGAGTCAGCCGCGCGGG
AGGGCTCTAGGCTGCACCCGGGAGGTTCGGGAAAGGAGAGCAGCTCAGGGATCAAACCCACGATAAAGAGGCGG
GAAGCCAAAGCACTGGGAGACTCGATCTCAGTGACCAAAAAATGTTCGCGGTTCAAATAAAGTTCTTCCCTTGCTC
TTCCCTAAGGCGAGGCGCCGCCACGGTCTGGGGTGCTGGACGCCACGCGAGGGTCCTGGCGCCTCTGGCTCCCCGC
CTTCTGGCCATGCGATGCGGACGCCGGACCCGCTTGGGGAGCGGTGGAGGGTGGGGCTGAACTCGGTGACGCCTCC
CTCACACCCAGCTTCAGTGGCGCCAACGCCGCTTCCCCGCACCCTCAGGGCG
293; SW vs I; chr4: 155881084-155881434; 4; LRAT; PROMOTER
GAAGATGAGGTGCAGAAGTAAGCTGGGACCTGTGAGCCTCAATTTCGGCCTCTTCTGCGCTGAGACCCAAGCGGAT
CTTGCTTGGCCTGTATGCGTTACTGGGGGAAATGGACGTGGGCCTGAGCGCGCAGGTGCGAGGGCGCTGCCCCGGG
GCCGACCACCCTGCGGGGACACTGTAGCTGTCATTCCTTCTTCTGCAGGCGGGTAGGGGAAGCGGTGGCCAAAGTG
GGAGTCGACCGCTCAGCACAGTCTGTCTGAGTGTTGACCAGGAAAGTCCAGGCTCTTTCTAAATCTCGCCGCAGAC
CTGGTGACGCATTCGCATGTATTTAAGGCGTTTGCACGCAGAACG
296; SW vs I; chr4: 177159356-177159814; 4; GPM6A; INSIDE
CTTTACTCTTGGAGAAATCCCTCACAACAAAGGACAAGGGAGAAGCAAGGGGGAGGGGCGCCCAGTGGACTTGGC
TGGAAGAAGTGAGCGGGCTGGGGTTGGAAGAGTAACTCGGGCTGCGGGCTGACGCAGTCGGCAACCGCGGAAGA
GCAGCATCTCCCCTGCGCCTGTGGATACGCCAGTCCAGGGATGGCGAGTGCTTTCTCCTCCCCAGCTTCTCCCTCGC
TCTTCGAGGTGACTCGTGGGACCCTGCGTCCTAGTGCTGGGTGTGAATCGGCTATTTCACACCCAGTTCTTCCCCCC
TCCGCCACACGCAGTCACATTCCTGGAGCTATTCCAAGCTGCCTCCGCTAAGCACCGAATAAGCGGACCCTGCCTG
GAAACTTGAGCGAAGCTGAACTGCGCCGAACTCCACCGTCCAGTGACCCGAGCCAGTGTGGACGCCCTTTTAATCA
CG
317; SW vs I; chr5: 140724146-140724826; 5; PCDHGA5; INSIDE
CGCTGTGCGAGCCAGGATCCGGGCAGATCCGCTACTCGATGCCGGAGGAGCTGGACAAAGGCTCCTTCGTCGGCA
ACATAGCCAAGGACCTTGGGCTGGAGCCCCAGGAGCTGGCGGAGCGCGGAGTCGCATCGTCTCCAGAGGTAGGAC
GCAGCTTTTTGCCCTGAACCCGCGAAGCGGCAGCTTGGTCACCGCGGGCAGGATAGACCGGGAGGAGCTCTGCGCT
CAGAGCCCACTGTGTGTGGTGAACTTTAACATCTTGGTTGAGAACAAAATGAAAATTTATGGAGTAGAAGTAGAAT
AATCGATATTAATGATAACTTCCCGCGTTTCCGGGATGAAGAGTTAAAAGTAAAAGTTAATGAAAATGCGGCTGCA
GGGACACGGTTAGTGCTTCCCTTCGCGCGGGATGCGGATGTGGGTGTGAACTCTCTCCGGAGTTACCAGCTCAGCT
CCAATCTGCACTTCTCTTGGATGTGGTAAGCGGAACTGATGGACAAAAGTATCCGGAGCTGGTGTTGGAACAGCCC
CTAGACCGCGAGAAAGAGACTGTTCACGACCTCCTCCTCACAGCTTTAGATGGCGGAGACCCGGTACTCTCCGGCA
CCACGCACATCCGTGTTACGGTCCTCGACGCAAACGAAATGCGCCCCTGTTCACCCCATCCGAGTACAGCG
333; SW vs I; chr6: 101023927-101024343; 6; SIM1; PROMOTER
CGAATCGGGAATTGGCATTCCGTAAGGGAGCGGAGGGCCTCAGGCCACTAGGCAGCTCCGAACGCGGGGAAGCGG
GAGCAAAGATGAGCTCAGAGCGAGGGTTGCCGGCGCCACCCTGGGCTGCGCATGAGGGGCCGTGGACCCGCCCCG
AGACTCAGTGACAGTCGCAGCTTAACCCCGTTGGGGGCGCCGCCCCGCTGAGGTGGTTGCGTCTCCAAGTCGTGAG
CCTCCAATAGCTGCTCCCGCTTTCGCGTCGCAACCCCAGGACCCCGGGAAATTACCACTGTGCTCGTCTGGGCGGA
GACCCGGTGCTTCGGAAGCCTGGGGCACATGCCTGGGTCCCACCCTAGACTCACCGCCTCTGGGCCCGAGACATGG
GAATCTGCCCTTGAATATCTGGGCGCCTTTGCTGTTA
367; SW vs I; chr7: 121727243-121727884; 7; FEZF1; DOWNSTREAM
CGCTCCACAACCCGGAAGGAGCTCAGGCCGGACTTGGCGTGAGGGTCCCTGCGGGGCTGGGTCACCTACCCGTCCG
CGATCACCCCACAGGGGCTGTTAGTAATCTCCCTGCTTCCTAGGCCGTTGAAAGCACTCAGGTCACCCACTCTCGGT
GACCAGAGCGGTCAGTCTTTCCCCGCCATGAAATGTGAGTTCGGGTGGCAGCCGCCGCGCCCCTGCAGTGTCCCCT
GCACATCTCCGGTCTCGGTCTCGCCTTTCTCAGCTCCGCGCCGCCTTTTCCTGGGCGCCGCAGGACTCATGAAGTGA
GGCGGGGCCACAGATTTAAGTTTATTCCCCGCAACTGTCGGGGCGGTCCCGGAGCTGGTGAGGACGCGCTACGGTG
GGTGCAGAAAGGAGATCCCTGGACTCTGTAGAACCTGGGCCCGCGGTGTGGACCGATGGTGGATTCTCCGCGACA
ACAGCCCTGGAGGGGAGAGTGGGCCGGGGGAGGGGCCACGGGGCCCCTGAGGCCTGCGGGGCTGGCCGCACTGG
CCCCGGGCCTGGCGGGGTGGTGGAGTCCGAAGCGGCGAGGCGAGGACCCCGGGGCCCGCTCCCCCCAGTCGCCCT
CCCGGGATGTGGCCCCGCTGCAGTAGCACTACG
376; SW vs I; chr8: 9798161-9799053; 8; hsa-mir-124a-1; PROMOTER
CGGGCTCCGGTGCGGGACAGGCAGCCGGAGGGAGCTCCAGACCCCTCCCCTCGCGCCCGCGGCCGCTGCGCGGAG
GTCTGCGGAGGGCGCCTGGCTCGGTCGGTCGCTCCTTCCTTGGCGGGCCCTCCCGACCCACGGTGCTCAGCCAGCC
CCATTCTTGGCATTCACCGCGTGCCTTAATTGTATGGACATTTAAATCAAGGTCCGCTGTGAACACGGAGAGAGAG
GCCTTTCTCCTGAGGAAGGAAAGGAGGAAGGAAGGAAGGAAAGGTGAAAGAAAGGAAGAGGGGTGGGTAGAAAT
GGAATAAGAAAACCAGGAAAAAGAAATAAAAAGCGGCGCGTGTGCGTGCGCACTGACAGCGGGGAGAGGGATGG
GGGTGGGGAACGCCGGAGGAAGGGACCACAGCATCCTCCCCGCCGCAGCTCCCCCAATCACACAGACAATGAGAT
AACAGCGACGTCTTCCAAAGGTCTTTGTTCTCCCCCATCCTTTCGCATCCAGGCTTTTTCCTGCAAAGCGGAGGGGG
TGGAGGGATGGGGGTGTGGGTGGAAGTGGGAGACGGAGGGGTGCCTCCCCCGTGTTAATTACCCCGGCTCCCCTCG
CCCCTTTCCCCGCGCCTCGCCTCCCCTGCAGCTCCAGACATGAAAAAACAACATCGACCCCACCTCGCCCCAGCGC
AGCAACCCACCCACCCATGTCGCCCTCTGCTTCGCCCAGGAAACTGAAGGGGACTAGGAGGAGGAAGAAGAGAGC
GAAGGACAAGGAGAGCAGCGGGGACTCGGCAGCGGCAGCCGGGGCAGGGCGCGCGGCCGCCCCTCTTTACCTCCA
TCGCTGAGTGGGGGCGCAGCCGGGCCGGGCGTGCCGCAGGGGCGAGTTGCCGCGGTCCG
380; SW vs I; chr8: 65661156-65662687; 8; BHLHB5; DOWNSTREAM
CGCGCGCGCGCGCACCCGCACGCAGTCTGCAATGCCCTCACCAGTCCCCGCAGCTGGGCAACCCCTCCAAGACCCT
GTGTTACGCTTAAATTCCTCTCCCTCCTATCGTACCCACCATCCATCCTTAAGCAGGGAGGCCGAGACTCACAGAAT
TCCCTTCTCCACCAACTCGCACTCTTGGTCTAGCACGCGGAAACCGAACCTTTCTACGCGGCCCGACCTTGCGATTG
CTCCAGCATGAGTGGCATTGACTTTTCCAATGTAATGACTTATGAAAGATATAATCATGTGTTAAATTGATCCTCCG
TTTAACTGTTAATGGTGTGCTCTGGGCACATTTACGTATTTGATGCTATGGTAACTAATTACCACTAGAAAAAAGGA
TAATACACTTCCCAGACTTTGAAAAATAATCATGAGTTCACTTCCCGGCTCGCAGCACATCGTGGCGGCCCTGCCG
GGAGGAGCGAGAAACGAGTGGAAACCCGAAAGTGTGCTCATGGAATGCAGGAGGTAATGAATCCTGGTCAGGTGA
GACTACTTAGTCAAACCTTTAATGAAAACGTGATTCACATGGAAAGCAGGAGTGGGAGGGAACAACTACACAAAG
GTGCCTTTAAACAAAATTAAATGGCAAAACCTGCTATCAGAAACATGAATAGGTTTCTTTCAATTAAAATGCAGGT
AAGACTAAATTATCCACGTGCCAAGCAACTTTACATCAATTAACATTTTTTTGTTGTGTGGGCAAAGTAAATGAGCC
CGGCTCCTGTGCTCTCTGTATTGGCATATACAACTTGAAAGTCTCTGGATTTCCAAGCCAATGAGCTGCAGGTTTAT
TTAAATTCGGCCTTTTAAATCAAACTTTAGACACAGTAGGTGGAAGGAGGGAAAGAGAAGGAAAAACAAGGAGTC
AGTAACTTCAGTTTAACAGTAGAAACAACTCTTACGGTGAGAGTCTTTTTTGCCCGGCAAATCTCTGGCTCTCTGAT
CGACTGGTGTTTAGAACCAAGGTTCCAGTAAACAGGGCTGGGTAGGATAACGAGGAGAGTGTTTGTGCTGGGACCT
GGCAGGACTGGAGGAGGAAAGGAGCCGCCTTCCAGCAGGTCCAGCATCCCTAGGTTGCAAACACACCAAATGACG
TCCACGCAGCACTCGCCCCACCGCGCTGTTTTTGCTTGCGCAGTGCTCCCGAAGGGATTCGGGATC
398; SW vs I; chr9: 121170908-121172141; 9; DBC1; PROMOTER
CGTCGGGAGCGCGCGGGGACACACACGCCACTCACTCAAACCCGCAATTAGCCCTTTGAGCCCCAGGCACAGGAA
CCCCCTCCCTAGAACTGGAGAAGACTTGGAGGCGGCGGGGCGGCCAGGTGAAAGCGGACAAGGGTGCCGGTAGGG
GGAGGGGCAGAGGAGCGCGGGGACGCCCCGAATGCGGCCCGGGGCCGGGTCCAAGGGCAGCGGGGCTGCGGGGC
GCTGCACCCGGCGCCGCCTTACCTGGAGTCAATGTCCGTCTTTGGCGGAGAGCTGCGGGAGGACGCTTTTTATTCGC
TCGGTGGGAACTTGGGAGAGCCCTGCGTGCAGCTCGCATTCCGGGCACGGCGCGGGGACTGCAGGCGTGGGGGTA
CCTGGCTCCTAGCAGCCTGGCTCATACTCAGCCGTAAAGTCCCCTTCGCTGGTCCCGAGGACAGGCATGAATCCCG
GCTCCGGAAGGCGGTCACTCTCCCTCTGCCTCCCGGCTCTCTCGCTCTCGCTCTCGCTGTTGCTCGCTCGCTCTCTCC
CTCTCTCGCGGGTTCGCTCGCCTGCGCTCTCCTCCTCCCCGCGCGCCAGATCAGTTTGCAGCCGTGGGGTCCGGGAG
CGAGGCGGCTGGCGAATGGAGAGGGAAGTCCAAGTGGCCGCGGTCTCCGGGCGGGGAGCGGCGGAGGCCGGGCT
ACGCCGGGGGAGGGACTTAGCCGGCGACGGCCGAAAATGAATGGGGGAGCGGCGGCCGGGGGGCGGGGAGGGAG
GAGCTGAACGCCGGGGTTGCTTCCGAGGAGTGTTTTGTACATCTTTAGGCGGAGGAGGAGAGAAGCGAGAGGAGG
AGAGACGGGAGGCCGCCTCGAGGGAAGGGCGGCCCGGGTGGGCTGGGGCTGAGCCGTAGGGAGTTTCGTGGGTCT
GGGGCGTGTAGGGTGTTGTGTCTATGCGCACGAGCATCCAAAGGGACCGCGCATACGCGCAAACCCGGAATCCTG
GTGCCCTAAGCGTGACTGTACTGTGTGTGTCGCGAGTACCTGAGTGTTTCTGGGGCGGCAGGTGTTTCCACGGCCGC
GTGGATCCATCGAGTGTGGGTTTTGTCTCGGGAGTTGCCCGTGTGGGTCCGTTTGTGTCTGCGCGTCTCTCTGTGAG
TGTTTGACACTGTTTGTTTCTCTCGTGTGTAGGCGAGAGCCCACGTAACCTCCCAGAATGAAGCAAGAGCTTCTC
403; SW vs I; chrX: 50040955-50041296; X; CCNB3; PROMOTER
GCGTGTCGCAGCTGTGCACGCTGATTGGTCCTCTGCTGGCCAATCACCACTGCACTTCATGACGGCTGTAGTTTTCA
AAATCCCAACTGGGCTGGTGGAGGGTCGCAGGTGTCAGGTTGAGACGGACCGAGTCGCTATAGTAAGGAGCTCCG
TTCATTTCCTTGGCCTCTCCTGGTCTAGGGGTGCGTCGATGGTCTTTGCTGTCTGTCCTCGATCTCGGTGTGAGGTAG
GCGCCTTTGGGCAAGTACTGCTGGCTCCAGCGTCCGGCTCTGTCGCCTGCCAGTGGGTCCTTGGGCGTCCTCCAGCC
CCTTGCTGAATTCTGTTGCTGGGATGCGCGGCG

TABLE 3
CpG islands hypermethylated in (bladder) cancer. Provided for each are: ID;
Comparision; Coordinates of selected regions containing relevant CpG islands based on
Homo sapiens full genome as provided by UCSC (hg18, March 2006); Chromosome;
GeneName; Location relative to gene Sequence.

2; ALL;; chr1: 2072175-2072389; 1; PRKCZ; INSIDE
CGGGCGCGGGAGCTACGCCAAGGTTCTCCTGGTGCGGTTGAAGAAGAATGACCAAATTTACGCCATGAAAGTGGT
GAAGAAAGAGCTGGTGCATGATGACGAGGTAGGTGCCGCTTCTCATGGGGCCGGGGGCCCGGGAACGCGCTGCCC
TGGGGCCTCCTCCGGGCTTTAGCGGAATTAATCCATGCACGAGAGACCTAGCCTCACGTTGACG
3; ALL; chr1: 2106293-2106715; 1; PRKCZ/FP7162; INSIDE
CGAGGAGTCGGTGTGAGGCCGCGTGCGTCTCTGTCGTGGACACGCGTGATTGACCCTTTAACTGTATCCTTAACCAC
CGCATATGCATGCCAGGCTGGGCACGGCTCCGAGGGCGGCCAGGGACAGAGCTTGCGCCGAGACCGCAGAGGGAA
GCGTCAGCGGGCGCTGCTGGGAGCAGAACAGTCCCTCACACCTGGGCCCGGGCAGGCCAGCTTCGTGCTGGAGGA
ACTTGCTGCTGTGCCTGCGTCGCGGCGGATCCGCGGGACCCTGCCGAGGGGGCTGTCATGCGGTTTCCAAGGTGCA
CATTTTCCACGGAAACAGAACTCGATGCACTGACCTGCTCCGCCAGGAAAGTGAGCGTGTAGCGTCCTGAGGAATA
AAATGTTCCGATGATGTGGAGCTCCTCTGATGCCTTTTTTC
6; ALL; chr1: 33385564-33385979; 1; chr1: 033385935-033385979; Unknown
CGGACGTTAAGCCGCGTCCAGGGCTCCGTGCAGGCGCTGTACTGGTTGCCATCGTGCATCACGATGCAGTAGAAGC
CGCGGCTGGGCTGGATCTGGATGCTGCCCTTGCGGCTTGCGGCTTCGTGTGCAGCCCGATCACCCACTGGGTCTTCT
CCGCCACCACCACCTCCCAGTAGTGGACGCCACTACTGAAGGCTTCAGAACCCAGCACCGACACCTCCACATCGAA
GCGCTTTGGCGAGTCCTGCAGTGGCTGTGGGTGCAGTTGCCGTAAGCCACAATGGTGCAGTCGTCCGACAGGATCA
GGCGCTGGTGGGCTGTGCCCGGGTCCAGGGTTAGGGCGGCTGGCACTGTGGGGGTTGGAGGAGGGAGAGAAGATG
AGTGGGGAGAAGGCTGTGGCCCACCATGCAGTG
8; ALL; chr1: 47672249-47672972; 1; FOXD2/MGC12982; PROMOTER
CGCGCCGGCTTTGCCGCTTTCCCTGAGGGTGTCCAGTTTTGTGAATAAAATTAGAGAAATCTGCGGGCGTAGTTCCC
CAAGCCCAGCTCAGGCTTCCGTTTGCAGCCCGGCGCTGGCCGAGCTCGAACGCTGAAAAGGGCCGCGTGCAATCGT
TCCGCTGTGCCTCAGGACAAACTCCGCTTCAAGCCAGTGCCGCTCTCCGGCAGTTTCCTGATTGACTTAATTAGATC
CGCATAATCGCTGGAGGGCTTTTCTTTCCCAGACCGAGAACAGCTCATTTATGGGGACCAAAAGGAAGAGAGGGA
GAGACGACCCGGACGCCACTGATAGCAACGTACTCTTCGCAAAAAACACTCAGTAACTAGTGGAAAAGCCCAACA
GGCATCCCCCTCCGCCCGCAACGCTTCCTCCCAATTGTTCTGGGCTGCACGAGGGAGATGGCCGGGCCGTGGAACA
GATGCAGAAGGAGGCCCGGCACGACCAGAGACGCTGTAACCAAGACCCGCAGAGCCTGTGGCTTCCCGCTCTTTG
GCGGTGCAGCAGGCATGGTGCTGGAGCCCAAGGCTTCTGGGAAAGAGTCCAGGTTTTCGCGGAGGGAGCGACGGA
CTCACGCGGGATCGCAGGCACTGCTGCAAGACGCCGAACAGGCTGAGCCGGTAGGCGAAAGAAGTAGGGTCACAG
AAGAAGCAAAGAAAACGAAATAAATTCCGAATCCCGCG
50; ALL; chr10: 21828640-21829645; 10; C10orf114; PROMOTER
CGTCCGGGAGCCTCCGCAACTCGCATTTCCCAGTCGCAACCCTGGGTCATTCCTGCCTCCGTCCCAGCTCCCGGACA
ATTCCCAAAGCCAAAGGCGTCGAGCGTGTTACTGGCTCCCACCAGCCCAATTAATAATCAGCTTAACTCGTGGAGG
CTTTGAGCCCCAGTGCTCCGTTCCCTGAAGCCTCCCAGTGCGGAGCCCGGCTCCTGCGAGGACATCCTTGGAGGGG
GGCCGGGGCGCAGGGACGGATTTCCTGGGAAAAATGTCGCTGGCGTGTTGCACCTAGAGGGCTGTGGGCAAGGAG
GCACGCACCAATGTCCCAGTTCTGGGCTCTCAGATCGGGATTCCTCCAAAATCGCGACAGCCGCTCAGGTGCTGGG
TGCTCACGTTGGCAGCACAAGCGCCAAATGCACGACCCCGCGCGCCGCGCGCGACCTCCACGCACACTTAGAGCCG
AGGTGACAAGGGAAGGCGGCGGGGGTGGAGCCGCTCTCTGGTGGAGCATGATCAATCACTTTAAAGGGTTCAAAA
GTTCAAGTTCCCCTTTGGAGGGGAGAGGTAATGCTTCCCGCGGCCTGCTTCTCTCTGAGACCCGCGGGTTCCCGGCA
GCCGCGCAACCACTGGGGATCTCGGCTGCCGCTTGCGGGAGCCCCTTCCGGGGAACACGCTCTCGGGCCGCGCAGC
CGGGCGAGGTTGACGCAGCTGCTAGGGGGCAGTTTGGCTTCCCCGGGAGTACCGGAGTCGCTCGGCAAAAACGTG
ATCTCAGCCTGCAGGGCCCGGGGCGCGCGCTTTTCCCTTTCTCAGCAACGGGAAAGGCCTGTTGGCAGAGGCCGGC
GGAGTGCTGGCGGCCTGGCGGCTGGAGGCTTCGGCGGAAACCTGAGCTCCCTGCGACCTGTTCTGCTAGCAGGGGC
GGGGGAGAGCCCCAATAAGCCGCCCCAGAGGCGACCGTGCAAATTCCTAAGAAAAAGCCCTCTAGAAAACACCAA
GCCCTTCCAAAAG
54; ALL; chr10: 22804715-22807056; 10; chr10: 022806090-022806137; Unknown
CGTCGGCGCTAAGCAGCTCTGGAAACGGGCAGACCCAGCTGTGCAGCGATGTCCAGTGTCGCCGCATCTGCCCCGC
GGGGTGCAGCATGAGTCTTCCTTTGTGGCGTGCGGCTCCATCGGAACGCGCTTGCGACGACAAATTCCTTTTTTCCC
CCCCGCAGTTAACAGTTCTGGGGCAGAGGCTGGTGGAGAGGTCCAGAGCCCACTCAGACCGAGATGAAGATGAGG
AAAAGCATGAGCAGGAAGAGGCTGGCGGCTGCGGCGCCACAGGGAAGAGCTCGGTGCGCGGCGCAGCCTCCCGC
GACCGCGACTCCTGGGCTGCGTCGAGGAGCCGCGTTGCCATAGGAACCGTAGCGGCGCCCCAGTGAAACCCTGCGT
TCGGACAGGAGAAGCTAACCGGGCCGCCCACTCCCACCCGCGCTTCCTCCCCGCCCCCACCGGCCGTGCGCGAAAA
GCAGAGATCCGAGAACCGCGTGCGGTACAAACGGCAAAAGCTTCGCGCGCATTTTCCGGGAGTTGAGCGCGCGGC
CGCAGGCGGGAACCTACCGCTCTCAGGCTCCCAGCCCGGGCGCTACGACCCTGTGGGCGCCGCCTGTGCAGCCCCT
CCTTCCAGCCCGCTCGGGCGCATCCCCCAGGCCGGGCCAGCGACGCGGGCACCGGGAGCCCCTCCCCCGGTCCGGG
CTTTGGCCCACACCCGGGGACCGCGGAGTGGGAAAGGAACCAAAGCGCGGCGCCTGGCCGACCGCGGACGAAATT
CGAGGCCGGAGGGCGTTTTCTTTTTTGCAAAATTGCCCCAAAGCCAGGGCCCATGTACCTACTGTCTCCTTTGCCCC
ACATGCTCCAAGAAAATAAGACACATTCTACCCCGAGTCCTAATTATTGGGCCATTTCCTTAACGCGCGGTCTGTCC
CCGTGGGCAGAAACATACTGCGAGATGCAGTTTGGTAATTAACAAAGAGACGAGACCTAACTGGGCTTCCGAAAT
GCTGGATACTGCGGCCGGGTCGCCCCGCATTCGGGCATCGCGCGGTTCCCGGCCTTCGGGACGTTCCGGCCCGGCC
GGACTTTGACCGCTGGCGAATTAGGAGAAACGCAGAAGGCGGACGCTCCCCAATTTCCCCATCGAGCCTTCTCCTC
CCGAGTCTGCGAAGCCCCTGGCTCAGGAGACACCGGCTCCGCGCCTGGGCCTGCAAATCCGCTTCCAGCGAGCGCA
GGCCTGTCGCTCC
87; ALL; chr12: 94776044-94776377; 12; SNRPF; PROMOTER
GTTACTTCCCAAAGGGCGTGTTCTCCAAGAACTGCATTTAGCATTTCAGATCAGAGTGACCCGAACTCAATAGGAT
TACGACAAAAACTAGTGAGATTCAGATAACACCGTTACATCGAAAGTTTTATCCGTGACAGAGGAAATATTGCTTA
CGCCCTTAGGTAATTTCAAATGCACGTATGAAGGCCGTGCGGGTTTCTGCAGGCGAAGCAAAAGAAAGGTGGGGC
CGGCAGGGTCGTGGGGCGGGGGTGGGGCATGACACCTATCCCAGACGCAACTCGACCTCGTTTGCAAGAAATTCTG
TATCCAACTCTCTTCAGTGCGTTTCGCCG
94; ALL; chr12: 113657930-113659205; 12; chr12: 113657930-113657987; Unknown
GTTCAGTGGTTTATAGAAAATTTCTTTCTCTCTCTCAGGTCCACTAAGACCGAGAGAGAGAGAGAAGTCGACTCTG
GCACACCCGGGCGAGGGGCTGCCGGGATTCGGGAGCTGGCGCGGTTGATTTTTCCGAGAATCCTCCACTTGGGGTG
ACGTCGGGCAGCGCGCGCGGGCCGTGAGGTTAATGCCCAGGCTTTTCTCTAAAGCGTCCGGGAATGATCCGGCGAA
TAAAACGGGTGTCTGCAAAGTTAATGAATTGTACAGGAGGCTGAGGGTGGGGACTTCGACCCGGGGAGCCAGAGG
CGGTTCTGGTGGACGCTTCCCCGTGCGCCTAGGGGTGCGCTGGGCTTTCCCAGCCGAGGTCTGCAGAGCGCACCGC
GTGAGCTTCATCGATCTCATCTTCTTCCAAGTGGGAGGAGGTGGAGATGCTGGGAGAGTTGTGGGGAGTTGGGAGT
GGGTTGGAGGGGGACTGTCTAGATTCTACTCTGGAGGATCCAATCACGTGGTGCTGTTCCCCTGCCTAGAACTTCA
AGGTTTCCAACGGTTTGAATAAAACCCAAACGATGCCCGATGTCCAACGAACGAATGATCTAGCCTCGGCCTACTT
CTCAGGACTTAGCTTTGTCCCTGCCCGCGACTTCCCGCTGGGAACGTCGTCCCCGCCACCACCGCTCTGCTTAGGTT
CGAACTCTGGGCCACTCTTCCTACTCCCTCTGGTTTCCAGCAGGAGAAGCGCTTCGGAGGAGGGGGCTAGGGGCGC
TCAACCTCCCGGGTTAGAGGAAATGCAAGTTGGGCCAATTTCGCCCTCCAGACTCCGAGGCGGATAGGCCCAGGGG
GCAGCGGCCTTGCCCGGCGGCCACTGCCCTCCTCCTCCCCCGCGCCCCCTGGCTAGGCTTTGAAGAGCTCATTTTAA
GAACTTTTCCATTTTCCTTTTGGAGGCGTGTTGTTCTTCTCAGTCACCTCGCTTTTTTTTTTTTTATGAGCTTCAGCGC
TGATCTGCACGTTCTTCCCTCCGTGTAATTTCAATTTGAACTCTCCCCAGAGCGGCCTTTTCCCTCTTGTTGCCTGCC
AAGTCGTTTAAGGCTAAGCAAACATTCTTCCTCCCTCCCGTCCGCGCGAGTCTCGGTCTACACCCGCGAGGGGAGG
CGGAGGCGGTTACTCCGAGAGTTCCGGTGGGTTGGAGCCTTTCAGCAGCCGCCTTCAGACTGTTTGGGAGCCGAAA
AGCG
99; ALL; chr13: 34950488-34951119; 13; MAB21L1; PROMOTER
CTTTGCCGTCATCGCGGGTGAATGCACCACTACCTTTTGGCAGTTGGAGGGGAGACCCATCAGAGCCGGAGAACCT
GCAGCCACGACGGCGAGGATGTGCAGTGGTCTCTGGGGAAACGCCGCCTTCGCCCTCACCCGGCGCGTGTGGCCCT
GTGCCGTCCGCTCGCCGCCTCTCTCTCGCTGCTCCGCATTCCCGTGCCCTCTTACTTTCCTCTCTGGGCTCAAGATCG
CTACGCTTTATTTCTGCGTGAGCTTATTTGAAACCTTACTTGGTGTCCCGGCTTACTCTCCACGTCTGCTCCAGACCA
CGGTCTGCCCACGCACCCCTCAGCCTGCACCGCCCACGTCCGGCCCAGGTGTGGTCGCAGCCAAGCCCCAGCCGCG
GGCCCCTGCGCCCTGGCTCTCTCCCTTGGGTTCAGGCCAGAGATGCTTGCCCCAAGACCTCGCCCCTTGCCTTCTTG
GGCCACCTGCAGAACACCCTTGGTTGTCAAGGACGTGAGACAGCCGAGGACGGGGAGGCCCCAGGGCTGTTCGGG
GACCTTAAGTGGGTCCAACGGGAAGGACGCGGGCCTGCGAGGGATGCCGGTCGGGCCTGGCTGGAGCCAGGCGCG
CAGGTGGCGCATCTTCG
102; ALL; chr13: 49595986-49600287; 13; chr13: 049599159-049599206; Unknown
CGGGCGGACGAATGGACGGATTCGAGCGAAGCTTATTTCCCTTCTCCTAACGCATCTACCTATAAATAGCGCTCAC
CGCGCTGAGTCACCAAAACCCCGAGTTTCCCCAAATAGCGCGTAACGACTGTGGGATCGCGTCCCTCGGGGCTCTG
TGACAGACTCCAACCACACACCCGCTGGGCTGGACTGCGGGCCCACACGGCCCAGCGGGTCTCTCACCACCCGGCG
CCCCAGGGACGCGGGCGAGGCCGCGGTGTCCGGACGAGTCGACAGCGTCGGACTTCCCGCGCGCGGACGGCGCTC
TGGGGAAGGAGGAGCCGGCGGGCGGCTGGACCTGGCCTTGACTGCGCGGGGTCCGCCCCCGCCCCCTCGGGAGCG
CAGAGTTGCGGGGGGCCGGCACAGCCTCCGCCCTGCGCCGCCCGCTCACACAATGGGACGCGTCGGGAGGGGGCG
GCCGCGGCTCCCATTCCGGTCCCAGGGCGGGGACTCCGGGCGGCGGGAGAAGCAGTCGGGCTGGGGCCAGAAGGA
AAACCCCCTCTCCCTCTGCCCACTCAAACTGCGAAAGGGCTCCAGCCGGCGGTGCCAGGGGCTGGGGAGCAGCTGG
GGAGGGAGCGGAAGTGCCACCCCCTGCAGTCAAGGGTGGTGACGTCACCCCTGCAGCCAGCCTGTAGGGGGGTGT
CTGTACATCACATGACCCGGCCCCCCTCTCCCCGCCGAAGCCCGCGCGCGCGCTCTCTCCCCCAGCCCCGACTCCCA
CCTCTCCCCCGCCCCCTCTCCCCAACTTTCGCCCTCCCCGCTCCGGCACCGTGTCAACAGCGCCAGCTCCGCCCCCT
CCCCGCGCCCTGTGGAACCGCCACGGCGGCGCGGCGGGCGCTCGCGCTCAGGTCCGAGGCTCCGAGAAGCGAGCG
TGCCCCGGTTCCACCCTGCCCCCCAGCTCAGTGCTGCTGGCTGCGTGGGCACCAGAGCCAGGGGGCACGGCGGCGT
GCAGCGACAGCGCTTCTCTGCTCCATCCTCCCACAGCAGTTAGAGGACGAAGCTGAGGCGCCGGGTCCGAGCTCGG
AGGGAGCGAGGCGCATGCACGGCCTCCGGGCCCTGACGCGTAGCCTCCGGGGGTCGGCCCACGGGCGAGAGAAGC
AGCCGGGAGCCCCTCCTCGACCCCAGCAAGGCGGCCCGCGCCTCCGCTCTCCCTACCGCCCGGGTGGCGCACGCCA
GTCCCCGTCTTTGGC
103; ALL; chr13: 94152191-94153185; 13; SOX21; DOWNSTREAM
CGGAAGCCTCATCCCGCCAAGCCTTCGCCTCCTCGCTGAGACTCTGAGCTGCGCTGGGGTTGGCGGGCACCCGATT
CCGCCCCGGCCCAGACCGGTCACTCAGTGTGTGCATATGAGAGCGGAGAGAAGCGACCTGGAGGCCATGGGTGGG
GGCGGGTGGTGAAGCTGCCGAAGCCTACACATACACTTAGCTTTGACACTTCTCGTAGGTTCCAAAGACGAAGACA
CGGTGGCTTCAGGGAGACAAGTCGCAAGGGCGACTTTCCAAGCGGGAGATGGTGAAGTCTTTGGACGTGTAGTGG
GTAGGTGATGATCCCCGCAGCCGCCTGTAGGCCCGCAGACTTCAGAAAACAAGGGCCTTCTGTGAGCGCTGTGTCC
TCCCCGGAATCCGCGGCTTAAACATTCTTTCCAGCTGCGGGGCCAGGATCTCCACCCCGCGCATCCGTGGACACAC
TTAGGGTCGCCTTTGTTTTGCGCAGTGATTCAAGTTGGGTAACCCTTGCTCAACACTTGGGAAATGGGGAGAATCTC
CCCCCCCGCAACCTCCCGCACCCCAGGTTCCCAAAATCTGAATCTGTATCCTAGAGTGGAGGCAGCGTCTAGAAAG
CAAAGAAACGGTGTCCAAAGACCCCGGAGAGTTGAGTGAGCGCAGATCCGTGACGCCTGCGGTAGCTAGGGCATC
CAGGCTAGGGTGTGTGTGTGCGGGTCGGGGGGCGCACAGAGACCGCGCTGGTTTAGGTGGACCCGCAGTCCCGCC
CGCATCTGGAACGAGCTGCTTCGCAGTTCCGGCTCCCGGCGCCCCAGAGAGTTCGGGGAGCGGTGAGCCTAGCCGC
CGCGCGCTCATGTTTATTCACGCGGCCTTGAGCAGCCGAGCTCCAATCCATATTAATCAACCGCTCGACCTACACAA
GTCTAAGTTTACGGGAGAAAACCTAGTCCCCGAAAGGAAGAACAGCAATCCGGACAAGCAGTTGGCGCCTTTGTC
CCG
107; ALL; chr13: 113930978-113931584; 13; chr13: 113931540-113931584; Unknown
CGACCCAGCCCCCGAGGAGAACCCAGAACCGACCCAGCCCCCGAGGAGAACCCAGAACCGACCCAGCCCCCGAGG
AGATCCCAGAACCGACCCAGCCCCCGAGGAGAACCCAGAACCGACCCAGCCCCGAGGAGATCCCAGAACCGACCC
AGCCCCCAATGAGATCCCACAACCGACCCAGCCCCCGAGGAGATCCCAGAACCGACCCAGCCCCCGAGGAGACCC
CAGAACCGACCCAGCCCCCGAGGAGACCCCAGAACCGACCAGCCCCCGAGGAGATCCCAGAACCGACCCAGCCCC
CGAGGAGATCCCACAACCGACCCAGCCCCCGAGGAGACCCCAGAACCGACCCAGCCCCCGAGGAGATCCCAGAAC
CGACCCAGCCCCCGAGGAGATCCCCAACCGACCCAGCCCCCGAGGAGATCCCACAACCGACCCAGCCCCCGAGGA
GACCCCAGAACCGACCCAGCCCCCGAGGAGATCCCACAACCGACCCAGCCCCCGAGGAGACCCCAGAACCGACCC
AGCCCCCGAGAGATCCCACAACCGACCCAGCCCCCACGGAGAACCCAGAACCGACCCAGCACCTGGGCAACATTC
TGC
111; ALL; chr14: 36205192-36206099; 14; PAX9; INSIDE
TCAGAGAATTTGGAAAGGCCTACTCTGAGGGGAGTAAAACTTCACCAGGCCCTGGGCTGGAAGCACAGGAGGTCG
CGGCTGGGCCCAGCGCCCTCGGGAGGCCAAGGGCAGGGAGCCGACCCAAGGTTAAGCCCTCCAGCTCTCCGTCGC
GGGTTTGGGTCCCGTCTCAAGAGTGGGGCGCGCGGGCTGGGCCTCCGGCCTGACACCCTCTCTTCTCTCCATCAGTG
AGCGACAGCTCCCCCTACCACAGCCCCAAGGTGGAGAGTGGAGCAGCCTGGGCCGCAACAACTTCCCCGCCGCCG
CCCCGCACGCGGTGAACGGGTTGGAGAAGGGAGCCCTGGAGCAGGAAGCCAAGTACGGTCAGGTGAGGAGGCGA
GGGTCAGGCCAGGTGGGCCGCGTGCGGCGGGGATTTAGGCGATGGAACACTTTGTGATGGGTCCCTTTCTGAGCTT
CCCGCGAGAGAAGCCCAGGCTGGCGTCCCTTTGCTGCTACGAGCCAGATCCTTCGTGGACTGGGGCGAAGCAGAG
GCCTGAGCTTGGAAGGCGGAGCTGGGGCCTCGACCCCCGCCAGGGGCCGGGAGCGCTCGTCAGGGCGCTGGGGGT
CTGGGGCGGCAGCTCCCCGGGGCGAGGCTCTGGGAAGCGCCTCCAGGCCTGTCGGCCTCCGGGAGCTTGGGAGGC
GGCTCCCGAAGCCCTTTCGGCGGCTCTCGTTGGGGGTAGAGTTAACCAAAGAAGGCGCTTCTGAAGGGCCGAGCGG
AGCAGCCCTGGGGCCTCAGAGCCGCGCCTTCACGCCCGCGAAACGCGCGCCCCGGTCTCGGCCCCACGGTGCGACT
GCGGCTGCGGGGTCCTCACGTTCGGACTTTCTCTCCGGTGGCTCTCGGACAAACACGCTTGGCCAATCCTGCG
130; ALL; chr15: 94688798-94689131; 15; NR2F2; DOWNSTREAM
CGCTACGAATGTAAGATGAATTTAGTGGCGCGGCTGCCTCCCTGAATCCGAACGGTAATGATGGATTTATCAACGG
GGATTCGCCTTCAGCCCAGCGAGGGAGGTTTACTCAGCAAATATGTCGGCGGCACCCACCCAAATGCAATCATTTG
ACCCCCTGCGGACGATAGCTGCCGTGCCCCTTCCCACCGCTGGTCCTGCTTGGCCAGGGTCGCTGGGGGCCAGAGG
CCGGCCCGGATGACCAGAGAGGTGGCCTCACGGATCCTTGGCCTCTCGCCCCCTCCTCTGTTTCCTCCTCTCAGCTT
GAGGCTCTCCTGGCAAGGGGGATCGAT
133; ALL; chr15: 94696311-94696840; 15; chr15: 094696796-094696840; Unknown
CGGGGGGGTGGGGCGTGGAGGGCGGCCTGAGTGCCGCGCCTGGCCCCTGCCCTGGGTGCGCACCAGGCCGCGAAA
GCCGGGGGCCGCCTTCTCGGCTGCTGAAGCCCGGGAGTCAGTCAAGCGCCCTTGGCACCGCGAGGGAGGCGCACC
TCGCCTGGTGCAGAGTTGCCGCGACCCGAGCGGGAGGAGGCACAGGACGCAGGGCTCCTGGCCTGGCTGTGGGCT
TGGCCTTGGCGCACTAAGCGCTCCCCATCTGCCCGAGCTGGCCTCCGCGGCCCTCTCGGCCCAGTCTACGTTCGTTT
AAGGACTTCAGCGCGAAGAAGAGCGAACGTGGCTAGAACCTGTTGACCTCTCCTCTGGCCTGGAAGTCCGCACAGC
TCGTGGCAACACTGGACGGACGGTTGTCAGGGGACGACCGCGGGCAGGTGGGGAGGAGGCCTTGGTCTGGCTCCT
GGATTCCACCCTGGGACGGAAGGGGAAGCGGAAACCTTGACCTCAGCCACACAAGGACACCTATCTGGCTGTCT
134; ALL; chr15: 94705727-94706054; 15; chr15: 094705808-094705853; Unknown
CGCCTGCTGCCTGCCGTGGGGCCGCTCCGAGGTGCCAGGAACTCCGGTAAGGGTCCCGGCGGCGGCAGCCCGGGA
GTGGGCAAAGAGGGAACATGCGTGTATTTCAAAGAAACCAGCGCGGTCTCTGACTTCCCTGTCTTCGCTCTCCACC
CCCGCTCCCCCTCAACACACACATAATATAACAGACGCGAAGATAATCCTGCCATCTGGCAAATCCCAAATTTGGT
GACCAAGCTCGGAAAGCAACAGCGACGGCTCATCCCGGAGTAGGTGGCTGGTCCTGCCGGACTCTCCGGAGCTGG
GGCAAGGGCCGTGGGCTCTGCCCG
136; ALL; chr15: 94712480-94712812; 15; chr15: 094712480-094712531; Unknown
TAGGACCTGAAATTGCAGAGACATAGAAAATCAATACGTGGTCGGGAGTCAACTGCGCGCGACCAAGGGCTCTGC
CCTCCCTCTCTCCGTTGATTTGGACGCTGGGAAGCTGCCCGCCGTCCTTCCCACCCCCTTTGCCCAGCCTTGGCAAG
ACCTCTCCGGAAACATACTCGCGTTAAGGAACTGGGAAAGCTTCCTGGCCCGGAGCTAGGAGCCCGACGTTGGGGC
TCCTTGCTGAGTGAGACGCTCAGTCCTGTGCTCCCGCCCCGCCCCAGGCGCCCAGTCCCGCTGGGATCCATCTCCTC
AGCCCCGGTTTCCCGCTCTGCGAGCG
145; ALL; chr16: 52873104-52882105; 16; IRX3; PROMOTER
CGCGAGGATGGGGGGCTCCACGTCTGGGGGCGCTGTGAGCAGGGCCAGAGCGAAACCTCAGTCTGAGTTTACGA
ACTGGGAAGGAGCCTGAACCCCAGAAACACCGTTAACAAAGAGGGCGAACGCAGGGAGCCGAGGAGACGTCTCC
TTGGCCTCTGGGGAAGGGGCTGGAGGAGCGAGCCAGCTGAGAAGCGGCTTTGCCGGGTGTCCACTAGCGGTTCGC
CAGCCCTCTGTGCGCCCGCAGAGTGACGCAGAGGCCGACCAGTCCCCTCCCCACTCGCCCCTGGGGCGCCGCCGCA
CCTTGATCACCCCCTTCGCGGAGGGAACTCCCAAGGCGCGGCGCTGGCCGCTGGGTCCGCATGGCCCAGTCCTGTC
GGGCTCCGCTCTGCTGCCGCCGACTTCGAAGGTGTGCAGTGTTGAGCCGTGTCTTGTTCCGTGGCTTCGAGGGGTGT
GCGCGCTTCCTATACCTCCCCGGAACGCGCGAGATAGTCACACGCGCCACTTTGAGGGTCAAACACCCCGCATCTG
GCCACATGTACCTTTACTAACGTGGGGAGGGGGCAAAAAAATGTGTCTCTCCTTCTTACCCCGAGGTGTCACTCGC
CCAAACACTCCCTACAACTTCTTCAAGTCAAACTTGGGCAAGGTTGGCTGGCTGACTGCGAGAGGAAAAGAGGGC
GCGGAGGGGGCGCGGCGCGCGGCCGGGTGTAGAGGCCACGGAGGCGAGGCGCCGAGCGTCCCCTTTGTCCTGTAG
AGGGAGCTCCAGCCCCAAATTTCCCTGCTGCCTCCCCCGGCCCGCCCACCCCGGCCCGCCTGGAGCCGGAATCCCG
GCTGGAAAGGTGCGGCGGTCTGACACCCCCGCAACCCCCGCGCCGGGGCCTTAGCAGGATGCCTCTTCTAGGGCAC
GTGGGAAAACCCACCAGGGTGCCAAGACGCACAGATCTCCCAGGACCCGGGCTCCCCAACTCTCAACTAGAGCTG
GGCAGTCACACATCATCTAGAGGATTATGTACCCCTAGGCGACCCTCTTCTCCATATCCGCCTCCACCGTCACCCCA
CACCCTGGCATCTATAAAGAGGAAGGGGGGCAACCGGGTTGGAAGGAGGATGGAATCTGGGCTTGCACAGCCTCT
TATAGGTAACAGTGAGAAAGCCTCAACTGTGTCGATCAAAGAATGGGGACGAAGGGAGTGATGAGAACACGACCC
CGCCACCGCGCAGGCTA
153; ALL; chr16: 86294154-86294408; 16; KLHDC4; DOWNSTREAM
CGTGTGACACTTTGGAACGCTTCTCTCGTTACAAACCAGCGTCTTTACTGAAGGTCCTTCCGCTGCAGAGCTGCGCA
ATGGATGTGTGGACAGAGCTAGTCGGCACGTGGAGGCTCTCCTGGTTTTCAGCAGGAAACGTGCTGTGAGCAGGAA
TCGCGAGTGAGTTCCATGGCGTCCCCGGGTCAGCGCGCGTGCAGGAATCGCGAGTGAGTTCCATGGCGTCCCCGGG
TCAGCGCGCGTGGGGTCCTGTCTCG
155; ALL; chr16: 87372445-87372729; 16; chr16: 087372616-087372660; Unknown
CGGCGGGGAATCGGGTGGCCACGCACGTCGGCCTAAGGGGCCCTGGCAGCGCCCGGCAGGACAGCCGTCCACTGC
ACACCTCAGAGCCGGCACCCGGGACGGTCAGCGTCCCCATGGGCCCGGCACCGCAGCTGCCCAGATCCTCACCCGC
ACACAGGAAGGGTCCCTGCAACCCAGCGTCCACCGTGGAGAAACATGAACGTGAGCAAAAATACGGCCTGCGGTG
GGTGTGCGACCCCACACAGAAGGGCGAGAGGCTCGTGGCCATCCCACACACTGGGAG
165; ALL; chr17: 44074280-44075233; 17; hsa-mir-196a-1; PROMOTER
TTTCGCTAGATCATTTGTTAATTGTGGGAAGGAAGGACGCATCCGGGGAGAGGAGATCCAGGGCTGGAAAAGTGG
GTGAGCGGCGCTGGCGCAGGCGGCCAGGAGAGAGCAGATGAACCGTTCGTTAGAGCAGCGAGGTAGTCGTGAGCG
CTGAGATCCAGAGACTAGGACCCACTCCCTCTCTGAGCAGCAAATTGGGAAGAAGATGCTCACTCGGTAAGGGCG
AGGGAGCCCGGCATGGCGCCCCACCACGGGCTCGGTCTTCTGCGCGCCAAGATCCCGCTTGGGGCGAGGCGTTGGG
TCAGCGTTTAGAGCCACTCCCTGCGCTGGTGGCTGGACATAGCCTCCCTATCCCACCTCATCTTCCCCCATCCCCGA
CAGAGGAGGTTGTGAATCTACGGCCCTTGACGTTGAGGCGTCGGAGGGCGCACCTTTGTAATTGCGGCCTCCCTTC
GCCCCTTAAGTGCCGCTTCTGGGCGCCTAGGCTGGATATGAAAGCCCCGTTCCTAATCCTCTGCTCTGGTCCCCTCC
TCTGACTGCTGGGACTCTAAGCTAGGCCCTCCCCAGGTTCCATCACTGCGGCGCCAACCCGCGGCTGGGCTGTCCG
CAAGAGGGAGTTGAAGGCGCGCGGAATCCCGAGGTGCAGCTGACCCTCCTCTCAACGCCGACTCGCCGCTCCCGCC
CGGCCACCTCCCTGTCGGGCAGACTTCCTGTTCTCCTGCTCACAGCAGGGAGGCAGTCGCCGAGCCGGTCAGCAGC
GTGCACGGAGATCTTCACTCTGCGCCCAGCCCCGGGACACAGGTGCAGTCTCCAGCGGAGCACTGCGGAGTGCGCG
CCGTCGAGCACTAGGGAATCCTAGACGGAGGACTTGGTCCATTCCACGCAGTCCCAGGCAGGTCCGCAGCGGAGG
GACGCAGCGGTCTCCAACTCCTGGTCACGACTTGGCG
172; ALL; chr17: 58865328-58865618; 17; CYB561; INSIDE
AGAGGAGGCGGAGACCTGACCCCAGAAAGCAGCACTCAAACAGATGCAGCTGCACCCACGCGCCCGCCTGCTGCC
AGAGCCACTCTCCGGAGCCTGCAGTCCTGAAGACGCCTCAGCAGGGGCAGGCAGAAGACACCCCGCGAACCCCCA
GGGCCGGCCGGGCGCATCACTGGGAGCCGGGGCTATCTCCCTCCGTCAGCGTCTTGAAGTCCATGGAGAGGGCCTG
CTCTTCCGCCTGGGAAGGCCGCTTCCAGTCGGCCCGGTCAAGATGTAGAGCACCGCCCCACCG
176; ALL; chr17: 73932992-73933361; 17; DNAH17; INSIDE
CTTCTGCAGACAGACAAGAGCCCTCCTTACCTTCCATGAAGAGTCCGTACACGTAGGAGCCCTCTCGCGGAGGAGC
GGTCATGTCCTCTCGGTTTTTCTTGGTCACCTCGACAGACAGACACATCTTTCCAGGGGCCACTCGTTCTTCCTGGCC
ATGGACTGCATGATGGCCGTGAGGAACGACTGGGGGTTGAAGAAGCCGGCCAGCCACACGGTGGTGGGCAGGGCA
AAGTCTGTCGTCCAGGCCTCGAGTTCCTGCAAGGCACACGAGCCGCTAGGAGGAGAGGACATAGAATGACATGGG
AACGACCGGGCCTTGGCCGTTCTGAGCAGGGGTCACGCCAGAACCTTCCACGGGCCACTCAGCG
181; ALL; chr17: 75531280-75531546; 17; TBC1D16; INSIDE
CGCCTCACGGGGCCTGCCAGCAACGCGCTGTGTGCAGCAGGAAGCCCACGCCAGGTCCTGACGGCTGCATCCTGTG
TGCTCTCCGGCCTGGCCGCCTCAAGACACGCCACCTTTGATGCACACGCGCTCCGGAGGCACAGGAAGGACGGGG
GCACTGGGAAGTCCATGTTTAGTCTCCTGACGTCGACCACACAAACCAGACGGAACGGTGTCCCCGGCCCAGGCTG
AGCGGAGCACACCATGAGGGCAGAGGCTACAGGTGATC
183; ALL; chr18: 5186244-5187389; 18; chr18: 005186349-005186393; Unknown
CGTCGGACGTTCACAACGACCACGCTTTTCCCCGCTTCCTCGATTAGCCTGGCCTCCCTTGCTCCAGTGGGGCGGAC
TCAAATGCGCACGCACGCACACGCCGCCAAGCCGGTCAACCGCAGACAAACTGACTCTTCGCCAACTAGTAGCCAC
GGAGAGCAGCAAAGGGACCAAGGTCCTTCACCGCCTGCTCCAAAAAGACACGGCAACCTCCAGCGGCGCCGCAGT
AGCTGCCTCTGTGCTGCAGCCTCCGGTCGGAGTTTAGGTCTAATTCCTTAAGAAGTATCGCTCCTCTGCCCTTCGTC
CTACTCCCCTTGGCTTCGCCCACTTCGCCTCCTTCTCCTCGGGAGGCCAGGAGGACCCGGGTCACTTTGCCAATTCT
GCGGAGGGGGAAGTAGCGTGAGGGAGATTCCCACGCAAGTAGCCCGAAGCCCACTACTCTTTGCCCCACCTCTCCA
CTCTCTTGCCCGTGTAGGTGCCGGTGCTGCTGGCGAAAGTCCCAGTTCTTAAGGCTCCTGAGCTCGGAAGGGGTGT
GAGGAGCCAGATGGGAAGAGAAGCAGCGGGACGAGGGCGAGGAAGGGCTGGAGATTGTTAGAGGACAGATGGGG
CGGGACTGGGGAGAGGGGCGAAGGCGACGCGCCGAGGCACTCCTCCGCCCCATGAGCACAGACGTAACTCCGAAA
CGCGCAGATGGGCCGTAAGGTAAAGAGGCCTTTTTTCCCTCTCCTCTCCGTCCTCTACCCTGCTCCCCTCCTAGACA
CTTGGTGAAAAAGCAAGGTGCGGTGTACCTGGAGCGAACACCATGATTCTTCCAGCCGCTACGCCCCCAGATTAAG
AGAGAAAGACAGGCAGACGGAGGATGGGAAGAAGGCCGGACTTGGCGGGGTCCGGTGCAGGAGGGCGCGCTGGG
CGGGCGGCGGGCGGGGCGGTCAGCACCCCGGACGCTCCCGCCGCTAGATCCTGGGGCCGCAGCTCCAGCCGCCGC
CGCGCGCTCTGCCTCCACAATGCGGCCACAGCGGCGGCGGGAGGAGGAGGGTGAATCCCCGCTCAGTCCATCCCTC
ATTCCTGTGCCAGCTCGGGACGTACACTGCTGCTTAGGGAGCAAAGCACTTCCACCGCTTTCACGCGACGAAGGTC
G
193; ALL; chr19: 4059891-4060207; 19; MAP2K2; INSIDE
GACCACAAAGCGCTTCCTCATCCTTTCCTCGCCCTTTGATGCCGCCGGCAACGTGACTCTGCGAGCAGCGGGGCAG
ACGCCAGGTCTCCCTCGCAGGCGGGAAAGGGGCTCCAAGGCGGGTGCTGCCTGCTCGGGTCACATGGCTACGTGG
GGGCCTTGCTCAAATTCACTTCCTGCCTTCATTACAAAACTGTCAAAGGGGATCGCACGTTTGCAGGGTGTCACCCA
AGCATTCTGGTTTTGCAAACGACGCTGTGCGGCAGCGGTCTGATACCTGATGAGCTCGGTGTGGCGGGGTCGGCAG
CATTTCCTCCG
209; ALL; chr19: 63784504-63785085; 19; MGC2752; INSIDE
CGGTGTCTGGAGGAAGCGGCGCGGACAGTCTCCCGCTCCACCGTGTCGGGCCTGAAGAGGCCGCTGAACGCTCGC
GATTACTGAGGCATCTGGTAGGATCGCAAGGCCATCAACCTACAGGTGAAGCCGAGGCGCTGTGGCGCTTTGAAGC
CGGAGAGAAGCTCAGTAGAATCGGGAAGGCGCTGGGGCTGTCCAACTCCACCTTGGCCACCATTCGTGACAACAA
GGAAAAGATCTGGGCGAGTTCCCAGGAGGCCACACTGAGGCGGCCACCAAGTTGACGCGTGGCCGCAGCCTGGTG
ATGGAGAACATGGAGCAGCTGCTGAGCATGCGGATGGAGGACCAGAGCCAGCGCAACATGCCCCTCAGTGTGGTG
CTCATTCGGGAGAAAGCCCACAGCCGTTCGAGAACCGCAAGCAGGAGCAGGGCGAGGGCGCCTAGCCCGAGAGCT
TCGGGGCCGGTCGAGGGTGATTCGCTGGCTTTAAGGCGCGCCACAGCTTGAGCAGCATCGGGGCCAGCGGCCCAA
GAACGCAGCTGCAAGAATCCAGCGTTAATGCGCAGGGTGATCCCGGAGGGCG
210; ALL; chr2: 19424445-19425131; 2; OSR1; PROMOTER
CGGCCGTTCGGTGCTGCGGTATCCCATCCCCCGCCTGTTTAAGAGAAATTGATCACGTTAAGCGGGCCCTGCGTCGC
TGCGCAGTGCCCGGACTACCGCTTGGATGGATTGACAAGGGAAGGACGCAGACACCTAAGAGCAACAAGGGTACT
TTTTAAGCGAGGTTACATGTGTTTAGGACTCGCGAGGACGTCACGTTTCGAATTTGGTAAAAGTCCTCGGTAGCTAG
GACCCAACAGTTCTGCCCTCCTGGAAACCCAATGAGGTGACAAAACTCCCATCTCATTACTGGGAGTCCAGCGCAC
TCCGCCACCGCGGCTGCAGCTTCTGCTAGCTCGGAGCCTGCGGACTCCAGCCGGCGCGAACTCCGCCGCGACTCTT
TCTGGCTCTCTCGCTCACGCCTTCGGGGCGGAACCGCGGGCGCCGGAGTGGTCCCAGACTCGGCTGGGACCGGGCA
GCCTAGAAGAAGGGTCCCCTCAGTAGAGACCAGGCCTCCAGCTCTCCGTCCGGCGCTCCGCTCCACAACCCGCCAG
TCATGTGAGGTCCGTCAAGGGAGCGATCCCTCCGTCTGCCCGGGCTTCCGGGCTGTGGACCGCACTGAACCCAGTC
CCGCAGCAAGCCCTGCTGCCGCCTTTTCCTGGGCACGCCTGCGGTGGTGACGGCCCGCTGCTATGTCTCCACG
212; ALL; chr2: 43251514-43251780; 2; chr2: 043251514-043251559; Unknown
AAACACAGTGCCGCAGTGAAAGTGGGACAAACGCGCCTGTGTGCATCCTGGCCGAGGTGCCGGAGGAAGCGTGGT
CGGGGCAGAAGTGTTAATGGGCCTCCTTCCTGCCGTGCCCTGCCCGTGCCAGTCCTCGGTGCTCATCCCGGCTCCCT
GAAATGCTCGCTTCCACTCAGGGCCAGCGCACTCCCTCCACGTCCCTGGCCGCAGATCTGTCCTGCTTTGACAAATA
ACCCGCTCAGCCTTCGGGTTTATGGTTTTTCCGTCG
214; ALL; chr2: 45249374-45251726; 2; chr2: 045251412-045251456; Unknown
CGCTCCCTTTCCCGCACACGCATTGCTGCCTCCTCTCGGGCTTGGTTAATGAGTCTGTGCGCCAAGCCAGGTCGCTC
CGGGCAGTTCGCGCTTCCGCGCCTCGGCACTCGGATGACCGTGTCCTATTGTCTCTCTCGTGAATGTCGCTGAAAAA
AAATAAAAGTTTGTTCTAAGCCCGTGGCATTTCCGCGCGCAGATGGAAAGGCAAGTGGGAGAGGAGGGTGCAGGT
GCGGGGGGGGCGCGGCGGCCCCTTCAGGCGCCGGGGCTGGGCTGCCCCCGGCCGGGAGCAGCTCCGCTCCGCGCA
CGTGGCTGTGGGCGCTGCGGACGGGGGACGGCAGGCGGGGGACGGCAGGCGGGGAGTGGAGGGTGCAGTGCCCG
CAGGTGCCGACGGCTTCCTCCAGAGCCCTTCGCGCCGCGCCTTGCCTGGCTGGTGGCGCGGGGAAACCATGGCAGC
ATTGCGGCTCCGGCGGGCGGGCTCTGCGCGGGGCCCCACCGAGCTTTCCGGGAGCCTCTCCCGCAGCCGATGGGCA
TCTAGGGCGCAGAACGAAGAGTGGGCGCCGAAACGGGTGTAGGCGCTGGAGGCCGACGGGGAGGCCCGGGGCGG
TCAGGCTTCTCGGTAGAGAGGGCCGTGCACCTCGCGGCCTGCGGGCTAAGGCGGGGAGCCGCTCCTGGGCGGCCG
GGCGCGCGGACTGGAGGGAGGGAAGCGCCACGGACTGGGGAACCGGGGTGACGGGGAGACGGGGGGGCTGGTGG
GGTGAGGTCGGCGGGCAGCAGCCAAGTGTTCTGCAGCTCCGAACCCCGCCACCCCTGGGCGAACGGAATGGCACC
CGCGGAGACGGGCGGGGACGGGCGAAAGCGGGTTTGCTCCGACCCGAAGCTGAGCCCGGGTTGGAGGGCGAGAC
CGTAGGCGAGAATGGCCGCGCGGGCTGGGAGACCCGGGCCGTATGGAGTGAGTGGAGCCGCCCGAGGTAGGGTAG
TTGGGGATGGTAGTTGTGAAGACAGTCGGAGTCTAGCTCCGGGCAGACGAAACACCGGGGCGCGCCAGGGTCCGT
TTCCTCCTTCTCTCTGCTTTCCCACCACCCTGTAGATACGGAGCCAGCAGTCGGGCTAGAGCGGAGGCTGAGGGTCG
CGGTTCCAAGGGCGGTCTCTCGCCCCGGGTCAGGCCCCACCCCGCGGTGGCGGTCGCGTGGCGCTGTCCTGGAGTT
GAGCTGTCCCGCGCCGCGGCC
217; ALL; chr2: 63134470-63134933; 2; OTX1; INSIDE
CTCGAATCTTGGAGACACTGGCTTCTAGTCGCACATCTGCACTTTCTCCCACCTGTGGCCTCTCAGGCTCGGCCGCC
CGAGGGAGTTTCTTTTATTCCCAGTTCGGCTTTCTTTTGCGAAGGCCGAGTCTGGGCCTGCCAGGGGCCTGCCCGAG
TCCTCTATCGCGGGTCCACGTGGCCACCAATGACCCGCGGCGCCCCCGCGTGTCCCCGCAGCCACTCCGCGGAAGC
AGCGGCGGGAGCGCACCACCTTCACGCGTTCACGCTGGACGTGCTCGAGGCGCTCTTCGCCAAGACTCGCTACCCT
GACATCTTCATGCGGGAGGAGGTGGCGCTCAAGATCAACCTGCCGGAGTCTAGAGTCCAGGTGCGCACTCCCCGGG
CTCCAGGGTCTGGGTAGGGAGCTGAGGCTGCTCGGGGAAGGTCTAGGAAGGCTCTTGAGTAGTTCTTGAGGAGACC
ATC
220; ALL; chr2: 66525936-66527222; 2; MEIS1; INSIDE
CGGTTTGGAGAGGGGAGCGCAAAGCGCTGGACGCATGCGGTACAGTGCCACGGCCGCCGGTGGGCTCCACTGCCC
TGGGGAGCTGAGGCGCGACGAATGAAGCACCAGGGCGCCTGGTGGGCGCCAGTCTCCGGTCTGGAGCCTGCTGGC
CTGTCCCTCCGGGGCGCTGAACCCCTAGTGCGGCGTCCTGGGGGCCGGGCAGGAAGGATGGCCTCTCCACCTGTCT
GCGAATGCAGCCCAGCCAGTTTGAGCCTCCGCAGAGGTGCGCTCCGGGACTGGGCGCTTCTCGTGCTGTGAGAACG
CTGGGCCTTGTTAGCTCATTAACCCCTCTGTCTCTAGGGCCCGTTGGCGGCACGGTTTATTTTATTTTACCTGTTTTC
CTCGGAGGGCGCGAAGACTCCACCCGCGCGGGGACCTGGGATCGACGACTTTGATACTAGGCGGTATCCCGGAGG
GCTAAGTCGGCGGAAATCCACTTGACCTTGTAGCGTTAGTCCTTTCTTTTCCTTTCCTTTCCTTTTTCTTTCTTCTCTC
TTCCTATTTATTTATTTATTTTAAAAATAGGATTAAGACACCAGTAGAAGCTGTAATCCCGTTCCTTCCCCCACTCCC
AGTCTTCCGGGCCGCCTGGAGGTCCCTGCCGGAGAGAGCCACTTTGGCGGGCGCAAGCCCCTGGGCGCCCTCTCCT
GACCGCCGCGCTCTCGGCTCGGCTTGGCTGTTCCGGGCTCCCAGAGGCTAGGGGAAGCGAGGGGCGCCAGGGGCTT
CCCGGCCTCAGCGTGGGGCGAGGTCCCGGCTGCGACCCCGGAGAGAGGGAGAGGGGGGCCATGATGCTAGGCGTC
GAGGCGAGGGTTGAGCCTGTTTGGCTTCAGAGAACGATGCGGGTTCGACCGGAAGCGGGGCGCGTGTTCTAGGGG
CCCTTGGGTGGATTAGGGGGCGCCAAGAGGGTTAGGCGAGGAGAGGCCTGGCCACCCAGTTGCTAGACTTTAGGC
CGCACTCCAGGAAGGAGCAGTCGGCTTCCTTCCTAGGCCGCTTCTATTGCGCGCCACTCCTTGAATCTAAGCATTTT
CCACTCCAAGAACCGTTGGGAGAGAGGGGAGAGCGGAGCAGAGTCTCTAGGCCCCCAGGGCCGCCTCCCGGGGTG
GCCACGCCTGCCGGTAGCGAGCCGAGATTAGCTAGGTCTGTGCTCGGGCCTCAGCGGTGGCCCTGGCTCTTCCCTA
AGCAGCGGG
222; ALL; chr2: 70984709-70985764; 2; VAX2; INSIDE
CGGGCGCATAACAGGCACCCCGCACATCGCACCGCGGACGCAGCGATCCTCCCATTTGTACCCTAAGGCCTGAGAA
GGCCGCTCCACACCTCCGCGTGCACAGTCCCTAGACCAGTGAGGGCGGGGAGGGGAAATCCTTTTGTTTTATATTG
GAAACCTTACGCCAAAGGCAGGCAGGAAGCAACCAAAGGAACAACAGGCCGCCTGAGCCGTGGCTCAAACAGCA
CAGGGCAGTGGCCGAGCACCAGGCTTTTCGGTGGAGCGGGATACCAGCAGGCAGAAGAGGTGGACAACTCTGACC
GCAGACTCCCTACAACCCCGCGATCGATGCTCCACCTGCACGCTCAAACGCACCTGTTTCCCAAATTAATCCAGAC
GCGAATTTAACGACCGATTTTGTAGATCCGGGAAATTCTGTCCACCAGCGTCACCCAACACAGCAACTTTTACAGA
CAATGCGTGGCGTCCGTTCCTGCTCACCCAAAGCGGCCTCTCTGGGCTGACAGCCACCCCTGCGGGTCCTCAAACC
CCACTCAAACCCTCCGAACCCCTGTGTCTGGGAGCAGTAGGGTGGGATGACTGCAGGATCCCTTGAAGAAGTGACT
CGCTGCAACCCCACCACCAGCAGCAAAAACGCTTTCACCCGGGAGCTGCATGGATGCGCGGATGGCGGGGGCTGA
GGGGACGCGTGAAGCCCAGGCCTCTTGCTTGGGCTGGGCGATTCCCGCCGTGGGACGGGTGGGATTGACCTTAGTG
GAGTCCAGTCCTTCGGAGAATCCGCGGGCAGTCGGGACCCACGCTCCGGAACACCCAAGTCCGAACTCGTACACAC
AGCACAATGGCGTGCAGCCGCCGGGGCCCTAACTCCCAGAGACGCGTCTGAAGAAGCCATACAAGGGGGCGCAGC
TTCGGGGCTCTGGCTGGGAGGTTACTGCAGGGACAGCGCCTCGCCTCCAGCAGCCGGGTCCAGGCTCAGGGCCGGC
CGGCCTCTGCTCGCTTTCTGCTTGGATTACCAACAGCCACTGGGCTTGGGGAGATCGGCCGGGCCG
230; ALL; chr2: 105864220-105865009; 2; NCK2; INSIDE
CGGCAAGACGCGCAGGAAGACCAGCGCGCGGGATGCGTCCCCCACGCCCAGCACGGACGCCGAGTACCCCGCCAA
TGGCAGCGGCGCCGACCGCATCTACGACCTCAACATCCCGGCCTTCGTCAAGTCGCCTATGTGGCCGAGCGGGAGG
ATGAGTTGTCCCTGGTGAAGGGGTCGCGCGTCACCGTCATGGAGAAGTGCAGCGACGGTTGGTGGCGGGGCAGCT
ACAACGGGCAGATCGGCTGGTTCCCCTCCAACTACGTTTGGAGGAGGTGGACGAGGCGGCTGCGGAGTCCCCAAG
CTTCCTGAGCCTGCGCAAGGGCGCCTCGCTGAGCAATGGCCAGGGCTCCCGCGTGCTGCATGTGGTCCAGACGCTG
TACCCCTTCAGCTCAGTCACCGGGAGGAGCTCAACTTCGAGAAGGGGGAGACCATGGAGGTGATTGAGAAGCCGG
AGAACGACCCCGAGTGGTGGAAATGCAAAAATGCCCGGGGCCAGGTGGGCCTCGTCCCCAAAAACTACGTGGTGG
TCCTCAGTACGGGCCTGCCCTGCACCCTGCGCACGCCCCACAGATAAGCTACACCGGGCCCTCGTCCAGCGGGCGC
TTCGCGGGCAGAGAGTGGTACTACGGGAACGTGACGCGGCACCAGGCCGAGTGCGCCCTCAACGAGCGGGCGTGG
AGGGCGACTTCCTCATTAGGGACAGCGAGTCCTCGGTAAGTGCGCTGCGCCCACAGCTCCGGCTGCAGGCAGTAAA
TGCGCCTTGCGCGGTGGGTCTGTGTGCCGCG
232; ALL; chr2: 111591678-111597436; 2; FLJ44006-BCL2L11; DIVERGENT_PROMOTER
CGACTCGGTGAAGGATGATGCCCGGAGGGTGATCTCGACCTTTAACATTCCACACACCTACCTCCACGCACCAATC
GCCGGAATCTCCAACCCGCGGGCCGCGTGGGCTTTCTACCCTGCACCGCTGAGCCGCGGCCACGGGAAGAGGCGC
GCTCCCGGCGGCCCAAGCTGGGAGCCAAGCTCTAACGGGTGTGGCGGGAAGTGTGGTGGCCCGCCAGCAGCTGCC
ACGACGCTCGCTCCACCGACGCCCAGAGCTGTGGCCGGGCCGGGGGCTGGCACCCGCTGGGCCGCCACTCTCGGG
GATTTTGGTGGCAAAGCGGAGGTCCCGCCGAGGCTGGCGAGGTGCGCGGCTGGCTGCTAGGAAAGAGATCCAGAC
GGTCGCCTGGTGCGCTGGATCCCGTGCCCTTTCCCTGAAACCCAGCCTGGCCTGACTGCAACCTCTCCCAACTTCAG
TGCCGGATCCCCTAGACAATCAGGGTGGGCTCCCCGCTGCGAGCGCGCCCCACAGCCGGGTGCCGCCAAAGGTCTC
CTGCTGTAGCGGTGACTCACATTCCCAGTGATTTAGAAAAACTGTGGTGCCGAGTGAAAGAAAAAAAAAAAAGCA
AACACCCTTAGACAAAAGAAAAAAGCTCCACTCTTTCCGCGAGCGGGAAAAAAGGTTTGGTTCAGCAGTGATGCT
ATTTCAGTGAATCAGACGCTCTGGGGCTCTGCAAGGAAACGCACGGACTGGGAGAAGGAAGTGGGAGCCTAGAGT
TTTGCTCTCGGCAAAACAAGAACTCGGCTTGTCCTCCCAGGCCGGCGGTTTCCTACGGCAAGACAAGGCGCGGAAA
ACATTTTTACCATCTGACGCTGTAGTCTGTCCTTTAGAAGAGAATAGGTGAAAAGTTTAATAAAGAAATTAAGCGA
TGTGGAAGTCGTTCCCGCTGCTTGCAGAGGGTGGGGAGCGGGCCTGGGCAATTTCGGAACTAGGAGAGACCACGT
CGGCAAAGCCTGTGGTCCCGAACAAGGGCCATGGATTTCCCATTTCTCAGATCCCGGCTGCCCGACTTCATTCTTGT
TTCTTTCAGCACAAGCCATCCCCCTTCTCCCTCCCTCCACGTCTCTTGGCAGAGACAGAAAGGGACACCGCTCTGCC
CGCGTCCAGCGCGAAGGAGGTGACCTAAAACCCATGGGCCCTTCTCACACTGCGTTCTCTGCGCTCCGGCCCAGTT
CCGGAGGACCTCCCG
238; ALL; chr2: 156892636-156892878; 2; NR4A2; INSIDE
CGGGTTGGAGTCGACATGGGCCCTGACGAGGGCACTGATCAGACTCACCGGGGGCGAAGGGGGAGAGGGCTCCTG
TGGGCTCTTCGGTTTCGAGGGCAAACGACCTCTCCGGCCTTTTAAACTGTCTTGCGAACCACTGCAAAGGAAGAGC
CCTGTTAGCGCCGCTTTTCCGAGCCCAGGCCCAGCTGCTGCCTCGGTCCCTCCCCGGGGAAGGCCGCAGCCGCGGG
GCACCAGGCTGAGCG
240; ALL; chr2: 156893736-156894685; 2; NR4A2; INSIDE
TCCTAACCAATTTCATTCTGAACAGGGAAGACAGCTCCTAGCACATGCAAATGACCCTCTTCCAACTCCGGCTCCA
GCAACTTCGGGCGGGGGCCAGCCGGGTCGGCTGAATGCGAGGGGGATGCGACCTGGCCTCCCAGTCTTTCTGCTTC
CCTTTCTCAGACACCCGGAAGTCCCCGCCGCAGCCCATGGTCTCCTGCAGGGCAGCTTCGGCGGACCCCGGAGAGC
TGGGCAGTCCCGGGAGAGCTGGGGCTGGGCTACTGCACCAAGGCAGAGGGCACACTCCGAGGTCCCGGGCACTAG
GGGCTCCCTACCTGCCTACTCCGCTCCCGCCATTGCTCACCTTAAAGAAGCCTTTGCAGCCCTCACAGGTGCGCACG
CCGTAGTGTTGGCAGGCCGGTTGTCCCCACACACAGCGCACAGCCCCTCGTTGGAGGGGGAGCCCCGCGACGGCG
GTGAGGGCACCTGCGTGTCGAGCAGCTGAGACGCGTGGCCGATCTGCAGGCCCGGGAAGCCCATGGACGCGGGCT
TGCGATGGGGTTGGGCACAGCGAAGGTCTGCCCGTCCACCACGTGGTGGCTGCCGGCGGGCTCCGGGTTCATGGGG
ACGTGCAGGGGCCCGTCGAAGCGCATCTGGCAACTAGACACCGGGGTGCCAGGGGGCGATTGCTTAAGGAGAAGA
GGGAGAGGCGGGAGACTGGCGTTTTCCTCTGCTCGATCATGTGCGTAGTGGCCACGTAGTTCTGGTGGAAGTTGTG
GAGAGATCCCGGGTCGTCCCACATGGGGCTGTGCTGCACCTGGAAGCCCGGGTGGTGGGCGTCGGGGGCGAGGAG
GGCTTGTAGTAAACCGACCCGGAGTGCGGCATCATCTCCTCAGACTGGGGGGGCAGGTGGCTGTGTTGCTGGTAGT
TGTGCATCTGAATGTCTTCTACCTTAATGGAGGAC
245; ALL; chr2: 171384729-171385226; 2; GAD1; INSIDE
ATACAATTACCCACACAACAAAGAGGGCCTCGGGAGAGTCGCCAGCCCTGGTAGCATATGAACCAGGCACCGGCG
TTGGAGCTGGCCAGGGCAGGGCGGGAGGAACGCGAGGCCTGCGCGCCTTTGTTGGTGCTTTCGCCTTGATGGATTT
CTTTTCTGACGCTTTCCACATTTCCCGCTCCCTTTTGCCGCCTGGGGGTGGGGGTGGGGGGTTAGCGGGCAGTTGTG
TTAATTTGCCCTCCACTCCCGGCGCCCTTACGGAATCCCGGTGGCCGCCAGTCGCCGGCGCAAATGCCAAGTCACC
GGTAGGAGACACAACAGAGGCGATGACCTGAATCCGCACGTGGCCTGGTTCGCCTGGAAGCTGCCAAAGCGAAGG
CAAGAGAGCCCGGGACCCTGTGTAAAACAGCAAGCAGCACGTGTCCCAGGCACACTGTTGCGGGCCCTTAGCCTA
GCTACGCATCCGCACCCCAGGCTTCAAAGGCGCGCTGGGCG
254; ALL; chr2: 176737610-176738187; 2; HOXD3; INSIDE
GAAAGAGATCCGTGGGCTTCTGACCCTGGTTTAGGCAAAACAGGGGGAGGCGCCGAGACCAGCTCGAGCACTAGC
GGATTTTGAGAGAAACTGACCGCAACCTCCATCGCCTTCCCCCTCTCTTTCACTTGGATGGGCTGACTCTACCCGTC
GGTGATTTACGACGATTGCAGCGCTAGTCACAGCCTGGCGCCTGGTGTCCCCTCCCTTCCCAAGCCCCCTCAGCTTT
TCCACTGCCACCGGCGTACAAGCAAGTGCCGAGCGGCCTCCGCAAGTCGGACTAGCCTCCCGGCGTCCGAGGCCAC
CACGGGCAGCAGATTTTTGGTCCCCAGCGAGGCTGCGCGCGTTCGTCCCGCCTCCGACCGCCGAGCAGAGCTGCTA
GCAGAAGCAGGCGCCGGTACTTTATATAATCCTGCTGCTCGCAGGGTGCAAGAGCGGGAAAAGTGCGGAGTAGGG
AATTCTTTTGCTGCGCTGCCTCCTACGCGGAGCCTGCTTTCCACTGCTGAAAAGTGCCGGGCCTTGGGAAGTGTTTT
TCTTTCATTCCTTACCGAAGCGTTTACTGCCGCCGTGGTCG
259; ALL; chr20: 35582018-35583550; 20; NNAT; PROMOTER
CGGCTGGCTACCGGTGCGCGGGGACCACACTCACAAACCGCATTCCGGTCTTCCCGCCCGAAAATCCGCGCTGTGC
GAGGGACCCACAAGACTGGCGGTCTAAAAGGGACCCGCCTCACTTGGAAACGGGCTGCGGTAGCCACAACCCTCC
GGTAGCGGTAGCGTAACCCCGTTCGGTGATCCAGCCGCACAGCGCAACGGGTACAAAGAACCCCACTGGCTAAGG
CCGACCTACCAGGGCTTGGGGGAGGGGAGCGGAAGACGAGGTCGAAACGACCTGTCCAGCAGAAAACTATCCCCA
AGCATATTCCAACCACTTCTCCGTAGAGCTCATTCCTTCCGTGCATACGAAGGGCGCCAATCCTTCCTGTATCCCTC
CTACAGATACCGGGTGACTACGCTTTTGCGCCCAAAGCGCAGTGCTCTGGCTCAGCTCCCTACAGTAGCGACCTCC
ACCGCAGATTCTCATCTCCTCGCTACCGTAAGAGAGATGTAAAGAATCAGACGGGTTACGCGCTCAGGCACCCTCA
TGAAGAGAGGACCTGGGCTTAAAACTCATTCAGTATCAGGAAAAGAGCGCCGAGCACGGGCCTATTATGCCAAAG
CTTCTGAAAGGGGCACCACGTTTTTTGCTCTATGGGGCAGATGACCCCTCCCTAATTTCGGTTTTCATCCATCCCCA
AGGTAGGCTTTGGAGTGGCACCGGAGACTGAGCTCAAATTTGCAGGCCAGGGACTGGGGAGAAGGGCGCCACACT
AAGAGACCTGCACCCCCATTCTCGCCCTGTACTCTACCCAGAGTCGTGGTCCCCTCCATTTTAAAGCAAAATCCAAA
AGCAAGCACGGCGGAATCTTCTGGAAGGGGGCTAAGATGGAACTCAGGAGGCGGGGGTCGGTATGGAAAGAGCA
GATGGATTATTTTTTTCCTCTCCTGGCGAATGAGGGCGCCCCCAGCCACCCCTCCTCATAAACACCCCCCAAGGCGC
GCATGCGCACTTAGGTGGCGGGCGGGTACTTAAGGCGCGGCCACCGCGGCTGCGGCAGTGCGCCCAACAGCGGAC
TCCGAGACCAGCGGATCTCGCAAACCCTCTTTCTCGACCACCCACCTACCATTCTTGGAACCATGGCGGCAGTGGC
GGCGGCCTCGGCTGAACTGCTCATCATCGGCTGGTACATCTTCCGCGTGCTGCTGCAGGTAAGTCTGACGGGGTTTC
GGTGGGAGAGGGT
267; ALL; chr20: 60061693-60062033; 20; TAF4; INSIDE
CGTCTAATCGATGGTGGGGCGAAGGGAGAGCCACGGCGGCCCTGAGTGCAAGCACCATCCTGAAACCAGGAGCCC
ATGAGGCCGCGGGAGCCGGAGGACCAGGACCGCCCTCGCACTGCCCTGTGACAGCTCCCGGCCCAGCTCTCTGCCG
CTCTGGATCCGTCACTAAGCAGCTCCATTAAGTGCCCGGTCAGGCAGGAGCGGCAGGGAAACACTGGGGCACCCA
CATCTGAACAGAGAAGATGCCCACCTTGAGAAGGTACCTGGAGAAGGGAGCCACGAGCGACGTTTATGCTGAGAA
GCGGCATCCGTGCACCTCACCTCCGCCCGTCCACGCCG
281; ALL; chr4: 9756136-9756429; 4; chr4: 009756149-009756196; Unknown
CGCTCGGGTGGCCGAGTTACACCTGGCTATAAGTCATCCCGTGCACCTGACTAAATGCATCCCGTGCACCTGACTA
AATGCGTCCCGTGCACCTGACTAAATGCGTCCCGTGCACCTGACTAAATGCTCCCGTGCACCTGACTAAATGCGTCC
CGTGCACCTGACTAAATGCGTCCCGTGCACCTGACTAAATGCGTCCCGTGCACCTGACTAAATGCGTCCCGTGCAC
CTGACTAAATGCGTCCCGTGCACCTGACTAAATGGTCCCGTGCACCTGACTAAATGCGTCCCG
284; ALL; chr4: 111780013-111780343; 4; PITX2; PROMOTER
CGCGGAGAGACACAGGGAGAGGGAAGGGAGTTGCGCTGAAAAGACGCAAAGATACGCGCGTGCAACTCCCTCCC
CTTTCAGGTTTCAGAGGTTTGCAAACCAGGGCTGAGAGGAAGGGGCTCGGGAACTCACGTTCCTCTCGCCCCCCTT
CTGTCTGGAGTCTCGCCCGCCAGAGGCTGGTTAACCCCAGTCCCGGCCGCCGCAGACACTGCGCTGAGCTTTTGGG
TCCTCGCCTTGCCCAGCGCCAGTGCAGCTGAAGTGAGAGCTGGTGGGAAATGCAAATGGCTCCTGGAGAAATAGA
AGATACAGAATGATTCTCATTCCCTCCT
302; ALL; chr5: 115810975-115811393; 5; SEMA6A; INSIDE
CGCGGCTGCCGCGGCTGGGCTTCCGCTTCTGCTGCAGCGTTGGGGTTGACTCTGGGGTGGGGAGGGCCGTCAGGTC
CAGGTGGTGCTGGTCTGCTTTAATGAGCATCTTGGCCGTGTTGCCGGGAGTGCGAGCTTGCCGTTGTGCATGAGTGG
CGTGAGGATGGCCTCCGGCTTTGGGTCTTTGGATTGAGTGTCCCCAAAGAGGCCGCTGAGCTTGGTGACGCTGCTC
ATGGAGCCCCGGCGCGAGTGGGTGAGCTCCTTCTCTTGCGCTGCACCACAGCCACGTCTTTGCGCCGATGATCACA
GACGCAGTAGACGGTGATGCCCGAGAAGACGGCCCCCATGACGAAAGCCAGGATGACTGCAATGGCCAAGAGGGT
GACGGGAACCAGCTGGTCGGGCCTTTGAGGTAACTT
304; ALL; chr5: 122461778-122463450; 5; chr5: 122462228-122462286; Unknown
CGGCCAAGGCTGGCGGCGGCAGGTACCCGGGTGCCCCGAGGGCCACCCGCCGCCCCGCTCTGGCGCATCCATCAGT
GGCCAGCTTTCACCGTGTCTGCCAGGCGGGACTCGCCAGGTGTCAGGCTCATGCCATAAGCCTGACACTTGCGCGG
GGTCGCCGTGGCTCCTCAGGGAAGCTGGGCCAAGGAGGCTGGGTCGCGCCGGCGGGCGCCGAGGCCACCTTCTCG
GGGCAGGCCATCCGCGCTGAGGCGCAGACACGGCGGTCCTCCAGGCCCATTAATAACCCGGAGGGCGCTGCGATC
CGACAGCGCAGTCGCCACAGGCTCTATTTCCCGTGGCCGCCTGCCTCCTTCCAGCCCAGTTCGAGTGGAGGGATAC
TGGGCGCCCACTACCGCCCGGCCGGAGGGTGGGGGTCCCTCTCGCGCAGGCTGCTTTGGAGAGTGTCGGAGAGAG
ACAGCTTTATCTACAGCTTCACCCATTGTATAGCACAACAGAATCGCATTTAGCCTGGAGAACAGCCGTCGGGGCA
GGAGTTGTGCCTTCTCTAACAATTAGGCTGATCGTTAAATTTGCTATTCCTTTCTTCCGGCGTCTAGGGAGAGCGTG
CACACCAAACCCTCCAGAACAGCACCCCGGTCCCCCACCCCGCCCCACCTGTCCCCGCCCTGCTTAGTGCCTGGAG
GGAGAACTGACTGGTCGCTTGAGGGACCCCATTTCGGGCGCGCCCTTGGAGTCCTGGGGCTGCGGCTCCTGCTCTA
CCGGGCTGCAGGCTTGCTCTGCCCCGTCCCGTGGCGTTTGGGAGGCAGTGAGGAAGCCTCCGAGTCTCTGGGTCGA
AGGAAGTGCGAATGGCACCCGCGATAGACTGGCCGTCTCCCCCACCCCGCCCCGTCTGGTGATTTTAAATCGTCTC
CTGTGTAATTGTAAAAAGAGGAAAGCTGAATACCAGACGACCAGTATAGAATCTCTTCGAGACAGCAGATTGGGC
AAGAGACGGGCTTTTTTTCGCAATACAAGGATAAACACATCCTGAAACTCCAGATAGAAACCTCAGGAATGGGCG
AGAGGGTCAGAAGGAACGAAGACAGTAGGGAAGAGAGAGAGAGAGACAGAGACAGAGGGAGAGATGCAGAGAG
ACTGAAACGGGAGGAAGAAGCCTGGAACTGTCGGTTCCGTGGAGAGCGGACATGCTATCTGCGCCCAGATCCGAA
GGCTTTTGGGGAGCCTAG
311; ALL; chr5: 134390896-134393045; 5; PITX1; DOWNSTREAM
CACAGAAGAAAAGGCAACCCCAGGCATAAAGAGGCTCTCAGCAGCCCCTGCCCGAACGCTCCTGGCAGGCAAGGG
CCCCATAGCACCCTGGGGCCTCGAGGGTCACCGCAGACCCGGCCTGGGAAGAACCCGTAGAGTCCCGCAGCCGCG
GAGGGAGGGGGTTCTCGCTAAGGCGTCCCGCGGACAACGCCGAGAGGCACAGCTTAGCGGGTGCGCACCGGACAG
GCTCGCAACGCAGGACGGTGCCCTCATGGGAGCCAGGCGAGCAGGTCAAGAATGGTGGTGGGGAAGCGGGGTAG
GCAGACCTCGGCGGGGGCTGCCTCGGCGCTCTGCTCCTACCCAAGGGGCCCGGTTCCCTTTCTACCCACAGTTCCCT
TTCTATCCGCCTCCCTCCTCAGCCAATGAAGCAGTCGGGGCTCAGCAGTGCTTAGCACGCTCGGACTATGGTTTTAA
TAGACGTACATGGACAAGTCGATATAGACAGATTTATTACAGTCAGTCCAACATACACAGGGACGCTGTAAACAG
GGGCGCGGCCGGAGAGCGGGTGTGCAAAGTGGGCGCAGGGCCCTGGGGCCGCGCCCCTTGCTCTGCCGGCTCGAC
TCTTGCACGGCGGGCGGTGAGGAGGGGGCTGTTCGCCCAGACAGAGGGCCACCTCCTAGCCCGGGAGCGAGCAGA
GGGCCTGGGCCTGCAGCTAAGCTCAAGGCTGGGGTGTTCTGAGATGGACCTCCCCCACCTCCCGCCAGGCCCGCAC
TGCCCGCTGTCGCTCCGTGGCCTTAATATAGGGCTCCGGGGCGCGGGGCCAGCAGGCTCTACCAGGCGCGCCGGGG
CCGTGTGCGGCTCCACTGAGTGCCCGATCCTGGGCTGGGGGCACCGTCCCCCCAAACCCAACGCCCGCCCCAACAC
GGATCCGACTCGAGCTAAGGCGCTCTCGGCCGGGCCTGCTCGGAGAGGGCAACTTGGTTTGTACGGGGTCGGGAA
ATCCTAGGCAAGTCCAGGCCCCGCACATCCAGTCCGCAGGCCCGGCCACTCAGTCCGGATCCAGCGCGGGGGGA
CGCGGGATACGAGGTCGTCCTCCCCGGCCGCTGGGCCTCGGCGCTCGCCTACCGCGCCGCGTGCCCTCCGCGGAGT
GGGCCTCCGGGGCCCGTGGGAACACACACACCACCCGCGCCATCCCGAGAGAAATTGCAACTCATCCATTTTTTCG
CGGCACTTTCTCCGACGT
314; ALL; chr5: 135442969-135443206; 5; chr5: 135443148-135443192; Unknown
CGCGCGGGACCGGAGGCGCACACCGGAGGGGAGGTGCCCCCACCAAAATTTAAGTGCGGCGCAACATGCACCACA
GGTAGGGGCACTACCAGGGCAAAAAGACTTGGTGAATGCGAGGGCCGGGCCTATCAAAGGTGCGTATGGTGGTAC
CAGAGCAAGGCGGAACAGCTGCTCAGTGGTCCGCAACCGGGTCAAGGGACAAGAAGACGATTCTGGATTGTAGGC
CGCCCGAGAGCG
319; ALL; chr5: 174091287-174092335; 5; MSX2; DOWNSTREAM
CGGTGACTCGGAACAGAGGCAGAAAGGATGCGAGGACTTCTTGCTGCGTAAACAGCGCGGCGGGCAGATTGGCAC
CTGGCACCTACCAGGACTGGCGCTTCGTCCCGCGTTTCCATTGTGCCCGCACCACTTTGAAAGTGGGCGCCGGAAA
CTCCGTTTTTCTTATGGGGCCGGGGTCGGGCAACGTGGTTCAGAGGTTTCACAAGCTCCAGCACAGAAGTGATCCA
AACCGAGGGGTCGGCTCGAAGTCGGAGGCTGATTTTTCTTGCTCAGTCGCTCCGCCGGAGCTGAGCCGCTGGAACG
ACCCAAACCTACCTGAAAGCGCGTCTCTGCAGTCCCGCTGGACTTCTCCGGCTCGAACTCCTAATCTCAGCCCGGA
ATCCAGCCATATTCATTTAAGATTGCGTTTTCCAAAGGCCAAAGCCTTTCCCGGTTTCGTCATCAGCCGCCAGGTTG
GGTCCCTAATTAGCCTGTCCACCGAGCTTCCTTCCCGGCGCCGCTTGGACCCCGCAGCCCCGGGCGATCTCAGCCCG
GGGGGGAATCCGGAGACGCCGGCTCCGGGTTAAGACTCTGCTCGTCCCCAGGAGGACGGGGTCGCGATCCAGAGT
GCGATCCGGAGAAAAGGGCTGCATCTTGCGTGGCTGGCGGGTTAGACTGGAATCCGTCTGGGGACTGCGCACCATC
CCCATCGCTCCAGACGCCGAAACAAAACCTGAGGCTCGGAGAGGGGAAAAAGAGTTGTTCAGGCTCGAACATACG
CATCCCAGCCTCATAGGTGGATGGGGTTACCGAGCTGAGAACCAAGCCGAGGTCCGCGAAGCCGGGCCGCCCCCTT
GCTCTGGGCCTCCCTTCAAGCCTGCAGTCTGGGGATGGGTTCCGAGTCACCGCGGTCCGGGCCTCGGTCAGGCTTG
GTCGGGCACACAGGGCGTTTCAGAGGCCAGATGACAACCTGTGGCGGCAAAAGGACTCTCCCTCACCAGGGACCC
GCGAAGGCGCGCATTGCAAGATGTTAGCGTGCGGGCTTTAGATATCCTTGCGTCCG
324; ALL; chr6: 1549606-1560865; 6; FOXC1; PROMOTER
CGGGGTCCCACGCGGGCGGCGCGGTCTTCCCCAGGCCTCCTTCTCTGGAGACCCGGTTCCCAGGGGACGCTGTGCG
GGGACAGGCGGAGCCGGCCCTTGGAGCCCGGCGCGGGCAGAGGCGCCGCGGGACAACGCCTCCGACGGCAGGGG
GCGCTCGTAGCACGCGCAGTGCGCGGTGCGGGCGCCCAGGACGGCTGCCTGGCCCCGGGTTCCCGAGGCGCGGGA
CCTGCCGGGGTGAGGTGCCCCCAGCCTGAGAGCCCCAGCTGGGTCGGCCAAGGGGCCTCCGGGCTTCCACATCGTC
CCTTTTTCTGCTGACGCTCGCGGGGAAGCGGCGGGAAGCCTGTGAGGGGCGGCCTGGGAGCAGACGGACCACCAG
GTCACCGGCGCGCGCTGCCAGGCCCGACCCACTCGCGAGGACCAGCGGTTCCCGGAGGCGTCGGCCCTCAGGTCCT
CGGGGGAGGCCGGCTGCACCGAGCCGGGTGTCGGCCTTGTCCCTGTCCTGAGAGGTGCAAACACCGGCCCCGGCCC
AGGCCCCGGGGCTCCTGCCCAGAAGGCTCAGAGCTGGGGGCCGACCGCGCCTTACCCGCAGGAGGCCCGGCGGTG
CGTCCGCGTTGGTGCCACTCCCCGGCCGGGGTGAGGGACGTCGGAAGGAAGGTCCCAGGGCGCAGGGTCCGCGAA
GGGAGCACTCGGAAGGAAGGCTGGCTGCGGCCCCGGCGCCTGGGTCCGCTGCGGCGCCGGGCCGGAGACCTGGGA
ACAGCCAGCCACCTGCAAGGTTGCAGCCCACCTCACCCCGCCGGCGGCTCCGGGCTGTCTGTGGGTCTGTCTCACG
GGGCAAAGCCTTTCTTCCCACACCCACAGCCAAGGCGCGTCCGTGCAGGGGCACACGCCTTCTGCTCCAGCCCCAG
GAAGGCGCTTTCGCCCTGCAGTCCTCCGACGGCCGGCTCCGCCGCACCGCGCACCCTGGCTCCGGCAGACTCTGGG
GCCTGGGGACTCGCCCACCCTGCGCGGCGCGCCCCCCACATGAGCCGAGGTTGGGAGGCTGCGGGGCCTCTGTCCT
CCCAGGCCGTGGAGTGCGGCGGCGCTCTGAGTCCGCTGGGGACCTGGATGGAGAGAAACTGGGGCTCGTTCGCGC
ACACATGCACCATTTGTTCTTTCCGATCATCTATCCCTTTACGAATTTTATTTTTATTCCGGATTGTTAATGCAAGAC
GACAGTTGAATCTCCA
339; ALL; chr7: 1481682-1482268; 7; chr7: 001481829-001481873; Unknown
CGGCCTGCGGTGGGGAAGCGGGTACGGGAGGCCCAGCTCTCCCCACCAGGGTCACTGGAGAGGAGCAGACGCAGC
CGTCCGCCAAGACCCGGGCACGAGTCCCCAGCAGCTGTCACTGCCCCAAACTAACGTGACCCCACGTCCACCAGCA
GGTGAACAGATAAACCAACGGCCGATCCACACAATGGCAGGCCACCCAGTGCCCACAACACCACGACTCTCCAGA
CTGTGCGAGCAGAGCACCCGCAAGCTCCCTCGGTATAAACTCAACGCAGACAAACCCACAGGGCCACAGGGCGGC
GCGGACTGGGAAGGGGCAAGAGGGGTAGCCGGCGATGGCCATGTACACTGTGGCCTCCGCCCTCCCTCCCTGCCCG
GCCAGGGCACCTGGGACGGCGGGGGGAGCGGCAGAGTTGCGCCCTCACCTTGCAGACGCTGCTGCTGGCAGCCCC
TTCGCTGTGCAGTAGGACCTCAGGCACGGGCACCCGCAGCTCCGGCCGCTGTAGGTAGAGCTGCAGGAGAAGGTCT
CGGCAGCCCTGCCCAGCACGCTGGAAGAGGTGGAGCAGGGCTCACCAGGCGGACG
340; ALL; chr7: 2082782-2083345; 7; MAD1L1; INSIDE
AACACAGTTATGACACAGTGAGCCCACAGCACAGTTAGCCTGCAGCACAGTGAGCCCGAGACACAGGTATGACAC
AGCGAGCACATGGCATGGTGACCCTGAGACAGGGTTACGACTTGGTGAGCCCCAACATGGTGAGCCCAAGACGGT
TACGACATGGTGAGCTCACGGCACAGTGAGCCTGAGATGGTTACGACACGATGAGCCCGCAGCACGGTCAGCCTG
AGACGGTTACAACATGGTGAGCCTGTGGCACGGTGAGCCAAGACAGGGTTACGACATAGTGAGCCCACAGCATGG
TCAGCCCGAGACAGTTATGGCACAGTGAGCCCGCGGCACGGTGAGCCCGCAGCATGGTGAGCCCGAGACGGTTAT
GACACAGTGAGCTCACGGCACGGTAGCCCGAGACAGAGTTACGACTTGGTGAGCCCGCGGCATGGTGAGCCCAAG
ACAGAGTTACGACTTGGTGAGCCCGCGGCATGGTGAGCCCGAGATGGTTACGACATGGTGAGCTCGCAGCACGGT
GAGCCTGCCATGGCCTCAAGGGTTAAGATGTAAAC
345; ALL; chr7: 2724511-2724848; 7; AMZ1; DOWNSTREAM
CTTCTCAGCAAAGGCCAACACGTCTTTCTCGAGTCCAAGACCATCTCCCGTTCAAGGGCCCTCACCGCGCTTTGTGA
GTCTGTCTCGTGGGCCTCCTTCAGTGGTCTGTGACCAGCGGGAATGAATGGGACGTGTTTTGTTCAGCCCTGACTCT
TGGACGCTGGTGGCAGCCGCGGGCGCCCACTCACCTGGCACGTGGACGAAGGTGATGAGCGCGGCTGACGGCTCC
CGGGGGCAGTGTGGGGTCCAGTCTGAAGCCGACGCCCTCTCGGTCAGGCTTTCAGCAGCAGAAGGCAGTGGACTCT
TCACCATTTCTTCCAACACTTTCTCACGTAG
349; ALL; chr7: 8449587-8450236; 7; NXPH1; INSIDE
AAACTGAGGCAACCAACTTATTCGCAAGAGTAAAACCCTATGGGGAGAAGCGATTCCTGCCCCTCTTCCCCCGGCG
AGGAACACGGCTGGAGCCACCCAGGCGCCTTCCAGGCTAAGGCGCCTTTAGGGCGCGCAGGGGTGAGGGAGAGGG
TGGGGAGAGTCCTAATTATTATGAATTTCTAAAGGCGCAGTAATTATTCACGGGGAGCAGGACAAACCATGGCTAG
GCAGGGAAATCGATATATTTGCTATCGAAAGTTCCTGCTCGCCTTTAATGCAGACGAATGGGGGATGCAGCCTCAT
TATTTTCCGTGGTTAGGCTCGCCAGCGTGGGGCCTGATGCAGCGTGAAATCTATCATCATTAGACCCGGGATGGAG
CGGCGGGGGGGAGTTTCTCTTACTTACCAAACCGCAACAACAAACAAACAACGACGAACAACCGCCCCCTACAAA
CACTCATTCTCACACAACGTTGCCCTACCTCCCTCGCCGCTTGCCCTGGCCGCTGTTGCACACTCCCCTGGGGGCTG
TCTGACGCCCTAGAGCAGACACTGCGGTCACTTAAAGTGCGCCCAGTTCCTCCACCGCAGCGGTCACACCGTTGAT
TTGATCCAGAAATAAGACGGATAGTACCGAGCGTTGGCG
354; ALL; chr7: 27164708-27165039; 7; HOXA7; PROMOTER
CGCCGCCGCCGTCTGTCTAGACTCAAGCGACTGAAGGGGCCAACAGAGCTGGTGTTTAAAGTAGAACCTGCCCAGT
CCAACAGCCCGAGCAGGGAGCGATTTCGGGGATCGCGGGAAGGAACGCACTCGCCAAGGGAGGGCCGGGTGCCCT
CGCCACCGGCTCATTCCTGCTCCGGTTTTGCCCGATGCGCGTCCAGGAGGTTCTGGCAGGACGCACTGCCCCTCTGC
CCCGGCCAAGGAGGATGCGGATACTGCCCGCAAGGTTCGGCCTTTATGGACCCAAGTCAGCCAACTGGGCCGAGTC
CTGCGGACACCGAAACCTCCCTTTCG
358; ALL; chr7: 27251165-27252762; 7; EVX1; INSIDE
CGCCTGTGCTCTGGACTCGCTGTGCAGGGTCCGGCTCCGAGGCGCTGGTCGGCAGTCCGAACGGAGGGAGCGAGA
CCCCCAAGAGCAACGGCGGCAGTGGTGGGGGCGGCTCGCAAGGCACCCTGGCTGCAGCGCCAGTGACCAGATGCG
TCGTTACCGCACCGCCTTCACCCGAGAGCAGATTGCGCGGCTGGAGAAGGAATTCTACCGGGAGAACTACGTATCC
AGGCCGCGGAGATGTGAGCTGGCGGCCGCCCTAAACCGCCGGAAACCACCATCAAGGTATGCGGGGTCCAGGCTG
GGGAGGCGGGTGTGCACCTATTTAGCGGGAAGTAAATGCCAACTGCCAACTCCCTGAAACGCAGGCCAGGAATCT
GGGCCTGGGGTCTCCCTGCCCGGGCGTGCAGATTGACCCTCGTGACAGCTCCTAGGCAGGCATTGCTGCCATGTGG
CTGACTCTGTCCCTTTCCTGGTTAAATAGACAAGGGGTGGGCGTGGGGGAAGGGGATAGAGTGCCTGTGCGGGTGA
CAAGGAATTTCTGGGGACACGCTCTCTGCGGCCGCAGACCAATTGAGTCCATGTCCTTTCACTGCTCCTCCCATACA
CACACTGGCCTCTGGCACCCCGGGGGCCTGGCCACCTGGGCAGAAGGAAGGAGGGAGCGGGCTGGATCACTCCCC
AAACCTCTCTCGGAGGGATTCCAGCTCCAGGTGGTGGTGGTGGGGTCGGTCTCTACCCGGCTGGTGTCTGCTTTGGC
TGTTCCTGCCCTGCGAACACTGTCCCCGGAGCGGGACCAGACTACTGGCCCTGAGCATCGGGCCAAGTCCAGCTAC
TGAACCTGCTCCGCTCCTCTCCCCAGGTGTGGTTCCAGAACCGGCGCATGAAGGACAAGCGGCAGCGCCTGGCCAT
GACGTGGCCGCACCCGGCGGACCCCGCCTTCTACCTTACATGATGAGCCATGCGGCGGCCGCGGGCGGCCTGCCCT
ACCCCTTCCCATCGCACCTGCCCCTGCCCTACTACTCGCCGGTGGGCCTGGGCGCCGCATCCGCCGCCTCCGCCGCC
GCCTCGCCCTTCAGCGGTCGCTGCGCCCGCTCGACACGTTCCGCGTGCTGTCGCAGCCCTACCCGCGGCCCGAACTG
CTGTGCGCCTTCCGCCACCCGCCGCTCTACCCCGGGCCCGCGCACGGACTGGGCGCCTCTGCCGGCGGCCCCTGCTC
TGCCTCGCCT
359; ALL; chr7: 32768010-32768497; 7; chr7: 032768257-032768301; Unknown
CGGACTTGTCCCCGCAGCCCTTGCGGACCTCCTGGTCGTCATGGCGACTGTGAAATGTGGGGTGGGGAGCATGCGT
TCGAAGCCATTTGCGCGGGCAGTCCCTGCGTGTCCCCCCACGTGCTCCCCACACTCGCAGGACCCCCGCCTCCGAG
CTTCCCTGAGCGTGCAGCTTCCAGTGAGGGCAGCCCCACGCACAGCCCCCCACACCCTCCCCAACGCCTTCAGCCC
CCGGTGCGCGTAGTCCCCAAGCCCACACGTGCACTTCCACCTTGCGGCAGCTGCACGTACAGCCCCCCACACGCTC
CCAGCCCCACTGGCGCAGAACCCATCACCGCTCGCCCTTCACGCGCTTCACTGGGAGTGCAGCCCCCACCCCGAGC
GCGCAGCTCCACGCAGCCTTCCACACTCTCCCCAGCGCCTGCAGCACCCCCAGTGCGCACAGCTCTGCCAGTCGCTT
CCCCGCGTGCCGCCCCTGCACCACTTCG
362; ALL; chr7: 55040457-55041024; 7; chr7: 055040965-055041024; Unknown
CGAGCACGGCCTCGGAGGCTGAGAGGCCTGGGAGGCAGGGCCGCGTCCTGCGGGCTCCACGAGAGCCCTGTGAAA
TGCCAGTCAGATCCTGGCCCAGTGCCCCAGGTCCTCCCGCAGCCTCGGCAAACCCTCCCCCGGCGCTGGAAACCTT
CCCCTGCCAGGCGCAGAAGGCCGCGCGCAAGCACGCAGCTCACAGTCACCGCCACCTGCTCTGAAACCCCAGGTTC
AGCGCAGGATCAGAGTCCCCCATCCCCACCCCACGCAAGGGACGCAGGATTGACAGCGACCCTGAAGAAATGCAG
TCGAGAGCTATTGTGAAAGGGCGCTGGCCCGGAGGCCCCCCAGCCCGCGCAGGACAGGCCAGTTAATCGCCTTGGT
CCCAGGCCCCTATTAGCGTGTCATCTGCGACAGCAGGACACGCCTTCCCATGCCAGCCGCTTTGGGGCCCGTGCGG
CCCTTTTTCCCTCGCAGCTGCTGCAACAAATAGCAGTTCTTTTGAGGGAAGGAACAGTGTGGTCCTCAAAGTTCATA
ATGAAAATCTTGGAGAAAAGTCATAGTTGCTAA
364; ALL; chr7: 96488158-96489591; 7; DLX5; INSIDE
CGAGCGGGACGAGCCCTGGGGCTCCCACACCGCTGGAGACTGCGGCGAGTTACACGCCATTGGGTCGCTGGAGCT
GGGACTGTGCTCCGGGGGCATCTCCCCGTTTTTCATGATCTTCTTGATCTTGATCTTTTGTTCTGAAACCAGATTTTC
ACCTGAGTTGGGGAACAAAGGCACACGTTACCGGGACACTCAGAGGTCGCCCGCGCCTTCCCCGGGGCGGCGACT
GGAGGCATCTTCGGACCTCTGGGCGGCCCAGCCCTCCTGGCGTCTCCCCGCCGCTTGCGGCCTACCGCCAAGAAGC
TATGCCTTAGGCAAACCATGGAGCTCTGGCCCCAGAGGGCGCCCTGGCCGGCTCGGGGCGCCCCGGCCCAGCGAG
GCCACGACCCCAAGGCTTGTTTTTGTTTGGGCTGGGGGTGGGGGACGCCAGCGCGGCGGGTGTGCGCCCGAGTGTC
CTCGGCGGCGGCGCGCCTCGTGTAAGCCTGCGCAGGACGCGCGGAACGCGGCGAAGGTCTCCAAAACAATCGCGC
GAGCCGGCGGGGAAGGGGCGTGGGGGGGGCTACGCGCGCCTGTGGTCAGAAAAAGGAGCGAGCTCCCAACCTCCC
GTCCTACTCCCTTCACTCCCTCTGCAGCTCAGAGAGGCCAGAATCTGACTGCAGCTCTTGGCGGCGCTCGAACATTC
TTTATGCTTCCGTAGGCCCCAGCTGGGTTGGAGGAGTAAAAGGAATGGGACAATCCAGGAAGACTTCTCACGTACA
ATAGCGCCCCCCACCTCGCGACCCTTCGGTGGCCGTGGCCTGAAGCTCCGTAGGCTCAGGGCACGACCCTTCAGTT
TCTCCGCTCCAGGCGACGTTCTCAGCCTTTTCTTAGCCTGAGACCACCGCGAGAACACCAGGCCTTGCTCACCCCCG
AGGAGGCTCTACATTGTTAAGAAAACCAGATACTGCTCTGGGCTGCCTAGGCCGCCCTAGAAATAACCCTCCGCTT
GCTTTCAACCCGCGAAATTGGCCCCACAGCTCCGGAGGCCCGCTACGGGGTGGGGGGGGGGGGCGCGGTTAGTGG
GAGGTATCTCCAGACCGTGATGAATACCATCCCCACCGTCTCAAGGCCTGACTCACCGAGTTAAAGCATAGGGGCT
GATGTCTTTGGGTCTGTTAGTTTCTCACGGGTACTGTCAAAACAGCTCCAGTCCCATCGAGACTGAACCGCGCGACC
AACCAGACGTG
369; ALL; chr7: 155266364-155266696; 7; RBM33; INSIDE
CGATTCAAAGATGCCGGCGTCCCGCAGTGTGCGGTTCGTGCCCTTAACCACCCGCTTCTTTGTTTCCCGCCCCTCTG
CTTTCGCAGGAGCTCTTGTGCTTGAGTTCAGTGTTAGTGGTAGCGTGGCTACTCCACTTGGAGGTGGCGGCCGTCTG
ACCGTGTGTTACTGCTTTGCCGACGGGGCCTCCCGGCCCTGATGCGTGTACACTCTGCGGGCTGCACCGGGTGGCTC
TGGTTGGGGGCGAAGCTGTGTTGACTGGGAGACGTTGGAAATTGAGACATGGAGAGATGACGGGAGTGCGTTTCT
CTGGGTTTGATCTCCATCCTGTTTT
371; ALL; chr7: 155288454-155292175; 7; SHH; INSIDE
CGCGGTGGCCCCCGCACCCGGAGCGTCGGCAGCACCTGGAGCGGTTAGGGCTACTCTGCCGCCGCCGCCCCCGCGG
TCCCCGCCGCCGCTGTCCCCGCCGCGGTCCGTGCGCGCGGGCGCCAGTGCACCAGGAGCGCGTGCGCCAGGCGGA
AGGGCGCGAAGGCCCGGTGCGCCCAGCTGTGCTCCTCGATGACCGCGTAGCACGAGGCCAGCACCCGGTTGATGA
GAATGGTGCCCTGGGCCGTGAGCGGCGCGTAGGCGCCGCGGCCTCCTCGCTTAGGGTCACGCTGTGCACAGCGGCG
GGCAGGAGCCGGCGGTCCCCGTCACGCTCGGCCACCACGTACACGCGCTGGCCCGGGCGCACGCGGCTGGCGAAC
AGCGCCCGAGGCCCCAGTGCGCCCCGGAAGGCGGCCCCGAGCCCGAGGACGCCTCGGGCTCCCCGGTGGCCGAGT
CGTTGTGCGGCGCCACAAAGAGCAGGTGCGCGGCGGTGAGCAGCAGGCGCTCGCGCGGCTCCCGCGTCTCGATCA
CGTAGAAGCCTTCTTGGCGCCGTCGTCGCGGTCCAGGAAAGTGAGGAAGTCGCTGTAGAGCAGCCGGCCCTGGTCG
TCCGCCGCCAGCACGCGGTCCCCGGGGCTCAGGTCCTTCACCAGCTTGGTGCCGCCCTGCTCCAGGTGACCGTGGC
CGAGCCCGGGAAGCAGCCTCCCGATTTGGCCGCCACCGAGTTCTCTGCGGGTGAGGAGAAGGGAAAGAAGAGAGG
ACAGGGCATTGAGTTCCGAGAGGGAGGCGCGTCTCGGGGAGGAGGGCGCACGCTGGTGCCCGCGCTCCTAGGCCA
GGGGTGCGCAAGGCGCGGGGCGGGGCGATTGATTCCAGGCGGGTCCCGCACACACCTCCCTCCCCCAACCCCACTG
CCCCAGGGACAGGATTCCGACACATTCCTTAACGACTCCTAAGTCTGGGCTGGCGGTCACAGAGGTTGGCTGGCGT
TTCCTGCAGAGGCCGGTGACACTCTTTCCTTCCGCCTTCCTCCAACCCCACCCCCAGCTCCGCACACACCCGGCCTC
GCCGCGAATCCATCCAGAGGGGTTTGCTCTGCACTTGGATTCCTCAGGAAGCCTCCTTGAGCTCCCGCGGGCGCCG
GGCGCACCCTCCTTCCCTCCCGCCCCGGCGCGGGGCGCGGCGCCTGCTCGCGCACTCAGCACCCGGGCTGCGCGGC
GGAAACCCTGGCCCGC
390; ALL; chr8: 142288380-142288718; 8; SLC45A4; DOWNSTREAM
CGCACGGCCTCCTCGTCCCTCTCGGGCAAGGCTATCAGCCAGAGCACCTCCCCTTTGACACTGACCGGCGTCCCCA
GCCCCGCCTTGTTACTGCATCCGTCTGAGGTACAAGGTTAGAAGCCACAGAACCCGAGCTGCAGCCTGACCCACAC
AAGCTGCGGGCGCAATGGCGCTTTGCAGACAAGACAGCCTGCCCGTCTGCCCTTCTTCTCGGCTTCCCTTAAGACAC
AAGGTGACGACACGGACGGTTTCAAGTGAGTGCATCTGGAAATAATCTCAGTGCAGGGGAGGGGTTGAGGCCAAA
GATGTCGTGACCTGGTCCCCGCTGTGGCCAGGC
402; ALL; chrX: 39749470-39754050; X; chrX: 039752648-039752692; Unknown
CGAGGTATAAAGACCAACGCACGACGGGCGCCGGGGCGCCGTCACCTGCCTGGGTTAACAGTTCCTGCCGCGGGG
GCAGGGAGAAAGGCGCTAGAGGGGCGCGGGGTGCCGGCGGGGCCGAGCCCGGGCGCGGGCGAGCCCTGGGTGCC
AGGCCCTGCGCCGCACGCCACTCTCGCAGCGCTGCCGCCGGGAAAGTGCCGGCGCTGGCTCTCCCTCCCCTCCCGG
GAATAGCGGGCGCAGGCACGCACATCCACACACATCCAACACACACACACACCGCACACTCACACACCCGGGTGG
CCGCGCGGGGCTCCGAGCCGCGCAGGCCTCCTTGGCAGACATCTTCCTGTGGGAGGATTTCAAACGAGCCGTGAAA
GCCAGAAACTACACGGGTTACAACTCCGGAATCCCTGGCCGGCCTCGGCCTCCCTCCCCACCTCCGGCCCGGCCCC
CTCCGTTCGGCCCGTGGCCAGGAGCCCCACCAAAGGACAAAGCTCACACGCCGAAAGCGCGGGGAGATCCGGGGG
CGCAGAGATGCCCCGACGGCCCTTCTACCTTCTGACCCTGGGACCCACGGCCAAGTGCCGCGCAGCCGAGGTCGGC
CTTGCTGATCTGTGGTGGCCCAGGGCGACCCGAAGCGCACCGGCTTCAGGGTGCGCCCTGGCCAGGGCTGGGGTGG
AGGGTCACATAAGCCACCTGGCATCCGAGGGTGGCCGCCTCGCCCCCTCAACGCCGCTCTCCAGGGAAGGAGGCG
GCGGGGTCACCAGGGCGCAGCTGACAGCCCCAGGCCCCGGCCAGGGCGCACCCAAAGAAGAGGGAGGCTGCTGCC
TGAACTGGCCTGGGGGCAGAGGGAAGTGGGCCCCGCGCTGTGGAAGGCCCCAGGCTGGAGCGCCGGGGACAGCTG
TCCAAGCGGCGAGGGCTAGGAGTGGCCTCGCCTAGCGCTAGCCGGCCAGGCCCTGGAGGTGCTCCGCGCCTTCTCG
GCCAAAAGCCAGCTCCGCGCCGCTGGCGGAGTGGGAGGGAGGCGGCCAGGCGAGCGCCCCGCCTTCGTCGCCCGC
GGTCACAGGGGCAATTACACTCCTGCTGCAGACGGTTACCAGCTCGCCCGCGCATCCCTCTCTCGGCTCCCCTCTCC
TCCCCTCTCCTCTCCTCCCCTCCCCTCCTCCGCGGAACTGCTGGCTCCATATTAGCGAGGGTGCCCTGGCAGCGCGA
CCGAGGCAACTACCCCT

TABLE 4
CpG islands hypermethylated in NMI-BC FGFR3 wt vs mut. Provided for each
are: ID; Comparision; Coordinates of selected regions containing relevant CpG islands
based on Homo sapiens full genome as provided by UCSC (hg18, March 2006);
Chromosome; GeneName; Location relative to gene Sequence.

4; SW vs SM; chr1: 2211959-2212429; 1; SKI; INSIDE
TAGGCAGGAGGAGGAGGAGGAAGAGGGCACCTGCTGAGATACTTCACTGGTCTGAGGGCCCCTTCTGCCCCAGCT
AGCGCTTGGGTGAGGGTTGGGCTTGCGGAGCCGTCCTGTAAAGCCGGGGATGCAGGACGCATTGTCACCCCCTCCT
GCCGCTCTTACGAAACACTCTTAATTGAGTCCGATTCTTGGTGAATCAGCCTTCCAAGAACCGCGACCGCAGCATCC
TGTGCCGCTTCTGTGTTCCGCATTTTTCTCTTTCTGCAGCGTTTCCTCTCATTCTGGATGGAAAGCCTGTTTGTCTCCC
TCAATCTTTGGCGAGGGTGGCAGGCAGCCAGGCGGCCATTACGGGCCGCGCCTCCCACCAGCCAGTCGCTGGCAGG
AGCGTCCGGGGAGGGAGCAGACCCCGTTCACCCTCACCCCAGAACTGAAGCAGCGAGGGGACCCAGACGAGCTGG
AATGCAGGCG
5; SW vs SM; chr1: 2226303-2228929; 1; SKI; INSIDE
CGCGCTCCGCCAGCAAGACCTGGCGTCTGCAAAACACAGGCGTGAGGGGCGTGCTTACTAATGCCTGGTGTTCCAG
AAGGCGGCTCCCGCCAGGCCTCCCGAGTAGGAAGGAGAGGGCTCGGCCCGCCAGACACACACACACCCACCTGCA
CACGCTGCTTCTCTGGGCTCTGCCTCACGGTTCTCACGCTGCAGCTCCGGTCTCGCGCCTCCTTTTACGTTGTTTAGG
TTTCTTTCTGACATGACTGAGGGAAGTGCGGTTCACAGGCAGAGCTGCCTGGGGAGAGAGGTGGCAGGCGGCCCGT
TGTTCACCGGGCCCCGGCCTCGCTAAATTCACACCTCGGCGGCGTCTGGCAGCTTCCGCGGCGTGGGCTTTAGCAC
AACACAACCCCGTCGGCCCTCAGAGCTGCAGGGTTGCCGACGTGCCCTCCTGCGCGTGCCAGTCCCTCGCCCCAGC
CGTCCCCCGCCACTGTTGTCCTTGATTCTGGTTGGAGGTGTGATTTCCCACATACATATTCATGTGGTGTGTTTGCAG
CGTGGGGTGTGAGTCACACGGATATTTTAATGGGGAGAGTGGGGGGGCTTGCGGAGGCGCCGGGCGGATGGACGT
GTCCATCTGGCTCGTGGGGAGACCCGGGGCCCTCAGATGGTGCGATGTCTGAGGCTGCCCTCCTCACATTGATGGG
AGCCAGGGCCTACTTGGCTTGCTGCCCCCAAGGGGAACGTGGCCCTAGATACCGGGGGAGGCCCCGCCGCAGCCTC
AAGTGACTGACTCAGGCCCCCAGGTTAGCACAGCTGCCACAGGCCTGGCGGAAACTCCGGATGGGGGTCTGAAAA
GCCTTTGTGGGGTCGGGGCACCACACGGGTTGGGCTGATGGCGCGCTGGGGGCGGGGCTCCAGGGCACATTGTCA
GATAGATGACCCCACGGGGTGGGCTGAGGACTGCTGGTCAGGTGAGGGGTGTGCTGGGACCGGCTGGGCAGTGAC
CCCGAGCCGCCTCCGGCCCCCAGGAGCTGGAGTTCCTACGCGTGGCCAAGAAGGAGAAGCTGCGGGAGGCCACGG
AGGCCAAGCGTAACCTGCGGAAGGAGATCGAGCGTCTCCGCGCCGAGAACGAGAAAAGATGAAAGAGGCCAACG
AGTCACGGCTGCGCCTGAAGCGGGAGCTGGAGCAGGCGCGGCAGGCCCGGGTGTGCGACAAGGGCTGCGAGGCG
GGCCGCCTGCGCGCCAAGTACTCGGCCCAGGTATGCGGGTGGGGAGACTGAGGCACGCAGCACGGTGGGCGTGAG
CCGCCGTGGGCCCCGGTGGCCAGTCCTGCCCAGCCGGAA
19; SW vs SM; chr1: 92718496-92725197; 1; GFI1; INSIDE
CGAGTGCACGGCTTTGTGCTGCTCCAGGCTCACCGCGTGCCCGAAGGTCTTGCCGCACATCTCGCAGGCAAAGGGT
CTGGTACCGCTGTGGGACCTGCGCACGTGCACCTCGAGCCCGTGCGGCGTGAGAACACCTAAGGCGGGTGGGGCC
AGAGAGAAGGCCGCTGAGAGGGGCCGCGGGGCGCAGGCGAGGCGCGGGTAGGGGAAGCGGGCGCACGGCAGGC
GAGGTGGTGAGCTCGGGAGCCTCACCTTGCTGCACTTGATGCACTTGTAGGAGCCGCCGCCCAGCAGCGGCGGGTG
CACAGCAGCTCCGACTCCACCTTGACGCCAGCGCCCTTGTCTGCGTGCAGCCCGTGGCCACGCTCGGGGTACAGCA
AGCCCGCCGCTGCCGTGGGCCTCTCATACAGCCCGGCTGCCGCAGACCCGAAGTCGCCGTAGAGCCCTAGGCCAGG
GCCAGGGTGGCACCGGCCCCTGCGCTGCAGCTCCCTGGCGCCCCGGCCCCCGCGCCGCCGGCAGCCCGCTTCGGGC
CGTACAGCGCGGCCGGGTGGCCAGGCTCCGGGGGGGGTTCGCAGAAGAGGCCCAGGCCAGCGCCACGCTCCAGGG
CCCCACACGGTCGGTAGCTTGCACCAGGTGCCGCAGGTCAGAACCCGCCAGGCCGCTCCATGAGTACGGTTTGAAA
GGCAGGGGGAAGGGCTGGGCTTCGTCCAGCGATGGGCACATTGACTTCTCCGAGGCTGTGGAGGCACAAGGGGAG
CGTCCGGTCAGGCTTCAGACGGCAGAGCGGAGGCGCCGGGCTGCGGCTGCGAGCGTGGCGTGTTCGCGGACTGCG
GGGCACCCAGCGCTTGTAGAACACTGCGTGCGCCAGGTGCCAGGCTCGCCCCAGGGTAAAACGTGGCCCGGGCCC
TGGGGCCTCGCAGGCCACTATAGGAGGCAGAACAGTAAACAGATCATTAAAAGACCTCGGGAGAGGAGGTCGTAA
ATTCTGTGCGAGTGCAGCGGAGGCGCTCGGCGTTCCGAGGATCTTTTCTGGCTGGACCCGCGCACCTGGAGATCCT
ACAGGGAAGGGCGCATCTATAAATGCCCGTAGTTGAGCTGTTAAGAAGGAGAAGGTGGCGGCGTTCGGCCCCTCA
CAGCACTCAAGGGCGGAAGGGTCCAGCCACCAGCGCAGGCACTCTCCCAGCCCCTACATGTTCCCATTCAATTCTT
TTTCTTCCCAGAATCCAAGAGGAAGGAGGGAGAAGGTCCATAAATGCGGCCCCGAACTACCCGAAAGGCTGCTGT
CGAATAAACCCGAAGGTATTAAGATTTCACGAAGTTAAGTGCC
24; SW vs SM; chr1: 163590111-163590435; 1; LMX1A; INSIDE
CGAGAGGCCCGGCCACCGGCTCGGGAGCTGGGCCGGTCGCTTTGGGAGCGGATGAGGAAGGTTAGGAGAAGCAGC
GAGATAGATCCCAATTTTACAATTCTATTTTCTTTCGGTAGGGTCTCGGCGTCTGGGCCACGTTGAGAGCGAACGTG
GGCCGAGCGGAGGACACAGAGTAAAAAGCGACGCCCGCTGTATACATAAATCCGCACCCGCTGCCCGCCCGGGTA
CTGCCTGCTCTGGCTTCCGCTCTCTTCCGAGGCTGGGCAAGTCCAAAAGTTCCCGAAAGGGGGGTCAAAGAGGGGC
GCTCAGTGCACGTGATTTCG
29; SW vs SM; chr1: 219134035-219134808; 1; HLX1; DOWNSTREAM
TTCTTCCCTCTTTACTCCATTCCCTAACTCCTGCCTCGTAGGCAGCGGTGGGGAGCCGCAGGCTGCTCCCCGAGTCC
CAACGCTACTAGGGTGCTGTAACAGTGATGAGGCGGCGTTGCAGTCTGGCAGGCGCTGCAGCGGGCACCAGCTAC
GTTTTGCTGTCGGCGGCAGTTACCCGGACGCAAGTTAAGGGGTAGGCGCGCAGAGGCGGTTGGAGGCGGGCGGCC
GGCCCCTGACCTCGGAGAGCTGCGCCGAGCAGGGCGGAAAATTTATGGTCTGTGTACACTTTCTGCTCCAGAGCCC
CGCGCACCAGAAGGATGGGGCCCTGCGAGCGCGCCCCAGGCCGCGAGCTGGGGCCGCAGGCGCCACGGCCATGTA
TAGTGCCCACCCGACTCTGTTCTGCAGCGCTGTGGTCATAAACCTTCCCTTAGAGGGCGCGGCTGTAAACGAAAGT
CCGGCGAGAGGTGGATGCAGGCCTTGCCGCCCGGACCCTCAGGTATAATTCTGGAAACTTGCACGGAAGCCTGGGT
CCCGCCATCTTCCCTGTTGTGGTGGGGTAAGAGGATAGTTTATACTGGCTGTTTGGAATCCCGCGCGGTGGATGCAA
GGTGGGATTCCAGCCAGCGACGCGCCGCGTGGCCGGGAGTGCAGACAGGTCACCGGTCCCCGGGTGTCTGGACCG
CGCCGCTTCCTACTGGGCTTACTTCGGGATCTAGACTTCGGCCTCCGTCCTTGCAAGGGCCCAGATTTGATTCCGGT
TCGTCGAGGCG
35; SW vs SM; chr10: 845627-845967; 10; LARP5; INSIDE
CGAAACCCTCGAGGAGCGACCACATGACCCCCGCGCTGTGAGCTGCTGGCGGCCAGCGCCCTCGCCTCGATTGTCT
GCAGCCCTGTAGTGGGCCGCGAGGCACGCAAGCCGCAACTACAGCTCGCCACGGGGCACACAGACAGGCATGCAC
CACCACTACCCGTCGCCCGGATACACTGAACTGGTTTTGAAGCCTGAACTGCAGAGATCCTAATATAGCAACGCAG
GACGTTTACGGCCACCCGCCAGTGACAGCCGGTGATGTGATGACAGGAGGGCCTGGGTGAAAATGCAAGACACTC
ACTCTCAGAGAACGACGACTCCACTTGAAATTCATGT
38; SW vs SM; chr10: 3150889-3151107; 10; PFKP; INSIDE
CGGCCGGGTGGGGACCGGCGTGGCGTCCTGCGGACCGCGTGGAGTTTGGGGTGTCTGACATCGTTCTCCACGTGGC
TATTTTCAGCGTTCTCCCGGGGAAGTACTTGGAAGAGATCGCCACACAGATCGCACGCACAGCATCAACGCGCTGC
TGATCATCGGTGGATTCGAGGTACGTTACCGTTTCTCTCTTGCCGGTCTTGCAGGTGTGAGCCGCG
42; SW vs SM; chr10: 7489383-7495345; 10; SFMBT2; INSIDE
CGGCAACCGAGCCAGATCCAAGAGGGGTCGGCCTCGTCTAAATTGGTTTCCCACCAATGGCCTCGGATCAGCCGCG
GCTGTGCTGCGGGAGCCCTCAGGACGCGGCTGGGGTTGGTGCGCGGGGCCCGGAACCCCAAACCCGGCTCGGTTC
GGCAAGGTTCAGGGAGACAAGGTAGAGAAGGCTGGGGTGAGCGAGAAGTCGGGCGGCCGATCGTCAGGGCCACG
AGCCTCGCCTTGCCTTCTTGGAATCCCACCCAACTTTAAAGGCCCAAAGATCCTGAAAATTCCGAAACGAAACTGC
GAGCTGGTCTCCAGAAGTTTGAGAACGGTCTCCCAGGCTTTCCAGCGTCGTCCCGGGATTCTCGGACACCACAAAC
GCCATCAACCACGAGCACCGGTGTCCGTGGCTATTGCCCCGAATGGTCCCCATCCGCGTCCCCGGGAACTCCCTCG
GCTTTCGCGCATCCAGGTCCCCAGCCCCAGCTACTGGTGCGCCCCGAGCCCCTAGGTGCCAGAGCGGTGGTCGGCC
GGGCTCCTGCCCAGTCTCGGCTCCTCCCTCCTCCCCACCAGAAGGAAAAACTTGGGCCCTTCGAGAACCCTGTGGA
ATGTTCTTTGTAATCAATGTACATCCGCTTCCACGGCACGGCCTCGTGCAAAATCGCGGGTTTCGGGGCCTTGGAGC
AAATTGCGCTTGTCAGCGGCGACGTCAGGAGGACAAGGGGAGGGGTTCGCGGCTGAAACTGCAGCTTCGCAGCAC
AGAGCCATTTTAGGCTGCTCCCCACCTCGCGGGCCCATGGGAAAGCCGGCCCCGGGAGGGCGCGCCCAAACGCAG
GCTGGAGGAGCCACGGACGCGTCCTGGCCGGCGTGTCGCCATCGTTCAGCCTCGCTGCCCAGGTGGGAGGGGTCAC
CTGCCGCGGGGTCTCCAAGCCAGTGCCGCTTGCTCCCGGCCCCCCCCACTGACAGCACGGCGTCCGAGTGACCCTG
TCTAGCCTCGTTCTGCGCTCCTGCAAACCACGTTGCTGCGCTAACTACAAACCTGGCCAACATGTCTTTGTAACCCT
ATCATTTAAAAACGCTTCCAGGCACCTGGCCGCTGCCAGATCAGGTTCGCGGGCCCGAGGAGGTCCTCCCACCTGC
CCCCGCCAGCCCCGGGGACCGTGCGCGGCCTCCGTGTGGCCCCCGCCCACGAGGTCCCTCGGGCAGGAACCGCCGC
GCGACCTCTGTTCAGCGGCCGCGTCCTGGCCACGGGCGACCCCTGTCGGGAACCCTGTTCCCGGCTAAGTGCGTTC
CCGCATTCCGGTGGCTCTCACCCGAGCTCGCGTTT
44; SW vs SM; chr10: 013553837-13554175; 10; C10orf30; INSIDE
TACAGTTGATACCCACCATCCCCATCATCGCTATTAGAATTGGGGTCAATACCCATCATCACTGTTAGACTTGAGGC
CGATACCCGTCATCACTGTTAGACTCGGGGTCGATATCATCATCACTGTTGACTCGGGGTCAATACCCTTCATCACT
GTTAGACTCGGGGTCGATACCCGTCACCACTGTTAGACTCGGGGTCGATACCCGTCATCACTGTTAGACTCGGGGT
CGATACCCGTCATCACTGTTAGACTCGGGGTCGATACCCGTCATCACTGTTAGACTTGGGGTGATACCCATCATCGC
TCTTAGACTCGGGGTTGATACTC
GTCATCG
45; SW vs SM; chr10: 13556159-13556753; 10; C10orf30; INSIDE
CAACATCTGTCATCGCTGTTAGATTTCGGGTCAATATCCATCATCGCTGTTAGACTCGGGGCCGACATCCGTCATCG
CTGTTAGACTCGGGGTCGACACCCATCATCGCCGTTGGACTCGGGGCTGAACCCGTCATCGCTGTTAGATGCGGGG
TCGATATCCATCATCGCTGTTAAATTTGGGGTCGATATCCGTCATGACTGTTAGATTTGGGGTCGATATTCCTCATG
ACTGTTAGACTCGGGGCTGATATCTGTCATGACTGTTAGACTCGGGGCCGATATCTGTCATCCTGTTAGACTCGGGT
CAATACCCGTCATCACTGTTACACTCGGGGCCGATACCCGTCACCGCTGTTGGACTCGGGTCGATACCCGTCACCGC
TGTTGGACTCGGGTTGATACCCGTCACCGCTGTTGGACTCGGGTCGATACCCGTCACCGCTGTTGGACTCGGGCGAT
ACCCGTCATCGCTGTTGGACTCGGGTCGATACCCGTCATCGCTGTTGGACTCCGGTCGATATCCGTCATCAGTGTTG
GGCTCAGGTCGATACCCGTCATCAGTGTTGGACTCGGGTCGATACCCATCATCG
47; SW vs SM; chr10: 15294510-15295293; 10; C10orf38; INSIDE
CGACAGCTTCACAGTAGGATTATTGTGATAAAAATGACTCAAGCGATGCAAAAAGTTTCATCTGTTCCCAGAATCC
GAGGGAGAACTGAGGTGATCGTTAGAGCATAGCGACATCACGTGCGGTTTCTAATGTCCCTGGTGGCGGATACGCC
GAGTCCTCGGAAGGACATCTGGACACCACTTTCAGCCACCTCCTTGCAGGGGCGACATCCGCCAAAGTCATCCTTT
ATTCCGAGTAATAACTTTAATTCCTTTCTAACATTTACACGGCAAACAGGAATGCAGTAAACGTCACGTCCGTCCCA
CGGCTGGGCTGCCGTTCCGTTTCCTCCACGAACGGGTACGCGCTTCCATGAGAAAGGATATTTGGCAATTTTATATT
CCACAGTCAGGTGGGTCTGCGATAGCTCATTTAATGTTAAACGCCATCAGGGGCCTCTCCTCCCGTTTCTGCCAGGG
CTTTTCTTGTCTTCTCCTTGGTCATCATCATCATCGTCTTCCTCTTCCTCGTGGGCAGATCTTCTCTGGTGGGGGCTGG
CTGCTGGCTCCGAGGGGGCATCCGCAGTCCGTCTGGTCGTCTCCTCCTGCAGGCTGGGCAGCTGGCCACCACTTCTC
CGACTCGCCCCTCCAACAAGCATCGCAGGGCACTGTCCTCGGGGGTACAGACCGTGGTCCCACATTCGCTACCACT
CTGTTCCACGTCATCCAGGTACACGAGCTGCGTGTAGGCCGTGCTGTCTGGGGCTCGAGGCTCTTTCTGCTGGTGCT
CTTGGACGGGCG
64; SW vs SM; chr10: 88112905-88117344; 10; GRID1; INSIDE
CGTGAACGCCCTCGCCTCGCACCTGTGGCGCCTGCCGCGGGGCCCGACCGGAGGCAGTGGCTGTGGACAAGGGCG
GCCCCGGCGTAGGCCAGGATTTCCCCGCAGCCGGACCGGCCCAGCTCCGCTCTTTCCTGGGCGAACAGCGCCCACC
TCCGGCCCAGGCGGCGGCGCCTCCGCCCGCGGCGCGGACAGCGAGAGAGAAACCGGCTCCTCCAAACCTGGTGGA
GGAGCGCTGCAGAGAAGCGCCGGGCTGAGGCTCTGGGGGCCGCGGGGCGCGAAACGCCGCGCCTACAGGTTATGC
CCGGGAGAAGGAGACAGGTCGGGCTCCAGCCGCTGGGCGCTCCTCACCTCGAAGCGAGGAAAACACCGGGCCACT
GAGATCAGGGGAGGTGCGCCCGATACAGGTACCCGGCACGCAGACACGCCCACCCCCAAACACCGAGAAACAAA
GACTGCCCGAGCCACGCACGCCTGCGCGGCCAGCGCTGGGCGCACACCCGCGCCCACACAGCCCGAAGGGCGCCA
CGCAACCCGCAAGAACACCAGCTCCCCGCGCTCGCAGGCTCTCCGTGCCGGCCCGCAGCGCAGAGCAGTCCCGGC
GCCGCTGCCTAGGTGGGCCGGGAAGGCGCGCCACCGCGCATGGCACCGCGGCTGCCGAGGAACAGAGGGTGCCCT
GCGCAGCCCAGCTCGTCCCAACCCCTCCGGTGCCCACTGCTGCGACGCCTCGCAGGCCGTGTCCCAGTGGGCCCTG
GGCACAGCGGTCACTCACCTTCCTGCACAGCCTGGATGGGTTGTTGGCCTCGATGACCTTGATGGAGTAGGTGATC
TTCTCGCTCTGCAGGATGTCATCGTTGAGGCTCAGGTCGGATACCGCCAACTGGAACACCCTGTCGTCCTTGGCCGC
GTTCTCCTCGAAGATGGCACCTGGAGGAGAGAAGGGATCAAGACCGGACTGGACAAGAACCCTCTCCCCGCTTCCC
TTGGCCCGAGGGTCAAGGCACTCGCAGACCGATGATTGCCGGGTGGTGGGAAGTCTGAACTGGGATTTCAGCTTGG
CGGGGTAGATTAAGGCTCTCCGATCTCCACCTGCACAGGAATATGTTCCCCTCCCCCCACCGCCCTCAGCGCACAC
GTGCACACAGTTGACATGAGTTACACATGCACATCCCACAGGCTGCCACCAGGTCACACACGTGTCTCACAACCAA
GGCACCACATGTCCTAGCCATGCTCCACACAAGGGACTAAGCCAAGCTGGGGTGGCCCCAGGCAGCGCCCAAATC
CTTGACATGGTTCCATAAATCCCATCCCAGGAGCCAGGAAG
71; SW vs SM; chr10: 102986025-102986636; 10; LBX1; PROMOTER
CGGGGGGCGCCGGGCGCAGGCCGGGCATGGGGCCTGAGCTTAGGCCAGGTGGAAAGGTGGAAGGACCTAGTGGG
GACAGACGAAGAGACCGAGGAGCCGCCCGCCCGGCTAAATGGACACAGGAAAGCCTGAGTCAGGCTTCTCATAAT
AAATAAACTGCACGTGAATAATTCAGCAGTCGCGCGGGGGCCGGTTGCATCAGCGCCGCCGCCGCCGCGCGGCGC
CCAATTCCCCGCGGAGGGGAGTAGCCAATTAAGGCACTTGAAAAGGGAGTCGGGTGGAAGATCCCCCGCCACCAG
TATCCTGGATTTACCCAGGTCGAGTTCAGAGAGCCTCCCCGGTCCCAACCCCCTCGGGCCCCATGGGTCAGGGGGC
CGCCTGCTGGGAAGTGCTACCTCAGCTAAGCCGCTCAGGTGTCCCGAGACTCCGGGCAAGGGAAGACAGGCGGCG
AGACGGACCAGAATAAGGGATGCGGACGTCTGACCTGTGGAAAACCCAGCATCTGCAGTGCCCAGCCGCACAGGG
AACGTGGGCGGCGTGGTTCCAAGCACAGTGAAGACGCCAGGCTCGGATCTGTGCTCAGAGCCGGGATCCCTCCGG
GCTGCTTCG
108; SW vs SM; chr14: 28317076-28317375; 14; FOXG1B; DOWNSTREAM
CGTAGCGACGACGTGGTCCTTGTAGGACCAAGCATTCTTGGCATTCTGTCGAGATTGAGATTCCAGAGCATCGGAT
GAACGGGACTTATAGCTAAGGCGTACTCACCCACCCCTCCCTTCGTCTTTTACGCACGCAGCAACTCGTCCTCACGT
GCATAGGGGTAGTGGACAGTGTTGACTTGAATTGCTGTCCCTCGTTCCAAGCCTTTCCTGTGAATGAACCCGAAAA
CACTCGACAGGTCGTGAATAATCGTTTTAATGAGTGTGCAAAGCGTGCGACGGGGTGCACCGACTGTCG
126; SW vs SM; chr15: 58079172-58079459; 15; FOXB1; PROMOTER
CGCTGGCGGGGGTAGGGGGTGTGGGTGGGTACGGAGTCCTTTCACTGCCCGTCGGCAGTTGGCACCAGAGCGGAA
AGTGTGTCCCCTCCCCCGCCTCTCTCCCTTGGGCAGTTTGATTTCCCGCCAGCTCAGTCGGAGTGGCCTGGAAGAGC
GGGCTCAAACTCCAAAGGCAGTCGTGGGAGAGTCGTAGCGGATACGTTTCCCCTGACATGCGCCGGGTGCTCGTCT
CTGCCCCCCACCCGCCCCCAAAACCCAAACCTGCAAGTCGATGTTGTCCGGTGACCCCG
127; SW vs SM; chr15: 58083339-58085812; 15; FOXB1; PROMOTER
GTGCCTAAAGTTACCTAACGATACAAACCGGATACAACGCAGAGGAAACAGAATACCCCAGCCAAGCAATTTCCA
TGTCAAACATCATCCGCGCGGCTGCTCCATCTGTGAACGTGAGTGGTCGCCTGCTCCCTCTTCCGCGGCGGCCGCTT
CCTCATACCTTCACACGGCGCACACCGGCCGGCGCGTCCAGCTGCCTGCCCAGTGGCCGAGGCTCTTCCCCCTCGCC
CAGTCTTGAGCTGGAAGTGATTCCTATTGGCCAAGGTGTCCATGTAAATAGGTGTGAAAGAAACAGAGCTGGCCGG
GCTCTCCCTTCTCGCTCAGTCCCCTCCCTCTGCAGCCCCCGCTCCCCCTCCTCTTCCTCCTCCTCCCAAGGCGATTGT
CATATGATAGCTAAGAAGTGGCACATTAATGAAGCGCCGCTACAGGGGTCTTTTCTGCTCCTGTCACCGCTTAAACT
ATCAGATGGTTCGAGGGAGGACATGGAGGCAGCCACCTAGCTCAGCGGAGACGCGGAGCCCACAGCAGCGCCCTC
CGGAGCCCTAACACGTCGCTGCCACCATCCGCGCCGGGACTCCGCAGCCGAGCTCGGCCGCCCGCAGGACGCTCCA
GGAGCGTCGCGACCGGGCGGCACGGGACGCTGCGGGGCTGAGCTCAAGAGCCCAGGTTCGCGCCGAGTCCAACCG
GACCCGGACGCTGCGCGCGGAGTGCGCGTCGAGTGCGCGCCGAGAGAGAAGCGGCGCGCAGCGGCGTCCTCCCGG
ATGCGGACGCGCAACTTGAAGCAACTTAAGGTGAGCAGCTCTCTGTTCCGTCCCTGCCCCCTATTCTGGCCCCAGTA
CCGACTTACTTCCCGGCTATCCTCGCGCCGTTCGCCGGCTTCCCCTCCCGCGCCCACTAAGCCCGCAAAGTTGCTGG
CGAAAGAGTCCGGGCGCTGGCTGATCGAGCGCCGCAGCCCCACCCCCGACCCCCGAAGTCTGTTACTCGGTCTGGC
TGACCCCGCCGGTGTCTCTGTGCATCCATGCTACCTTTCCCTATTACCCACCCCCTTCCCAGATCCGAGCAGTCCGC
CGGCCCGCGCGGACCCAGAGCAAGAAGAGGGCGAGGAAGAAGATGCCTCGCCCGGCCGCAACACGTACAGCGAC
CAGAAGCCGCCCTACTCGTACATCTCGCTGACCGCTATGGCCATCCAGAGCTCTCCCGAGAAGATGCTGCCGCTGA
GCGAGATCTACAAGTTCATCATGGACCGCTTCCCCTACTACAGGGAGAACACGCAGCGCTGGCAAACAGTCTGCGC
CACAACCTCTCCTTCAACGACTGCTTCATC
167; SW vs SM; chr17: 47590175-47591465; 17; CA10; INSIDE
CGACGGGAAAACGGGGGGAAGGGGGGAGCCCGACACGGCACACACACATACACACTCGCACACACTTCCGAGCG
AGTGCACACTCGCACTCCCACCCGACAGCCGGCCAGGGACAGTCACCCCCAAGTCAATATCGCAGTTTGAATTGTT
CCGGCAAATCTCCCCTCGGGCTCGACGGATGTGCGCCCCAGATGTGCTGACACATGTCCGATGCCTCGCTGCCTTG
GAGGTCTCCCCGCTCGCGTGTCTCTTCTCTTCGCACCAGCGGCGGAAACCGCACTAGCAGCGGCGGGGCGGCGGCG
GCGGCAGCAGCCACCTGAAGCCACCAACACTGGGCTCTTCCAGCAAAAACGAGACCCCGATTCGTCTGGCGCCCCA
AGAAGACATAGACGATAGCCCCCCGCCGGGCTGCGCCGTGCAATTCCGGGCTCCCGGGAGCGCGTAGCTCGCTGG
CTAACCCAGGCAGTCCGCGGCAAGTTGCCCTCGAAGTCCGCCCCCCTCACCGGGGCATGGTCGGAGGGTGGCTGCT
GCGCTCCGGGGCAGTTCTTCTCCTGCTTCCGGGGGGCGGGGAAGGAGCAGACACAGCAGACACACAGAGAAAGAG
AGGCAGGGATTATTGCCCAAACCGCCAGCCGCCGGCAGCCTCCGCCGACCCTCCCTGCTCCCCAGGTCCGCGCGCA
GCCTGCAGCCTCTCAGCCGGGTTCCGGCAGTGCGTCCAGGGCGCCGAGGGGAAGGGCTGGGCCCCGGGGACTGGG
GCGCGGTGGGGGCACACAGACTCTCCAGGTCGGCGCGCCCTTCCTGCTCCTCTGCTATTTTCCTGAACTCCCCAGAA
CCCCCAGTTGCCTGGCTTCCATCGCCCGCAACTAGCGCCGCTGTCGAGATTTTCCGCAATACAACTGCAGGGGTCGT
TATTTCCTCGCCTACGGATCCCTACGCTTCCCAACTCAGAGAGAGGAGGACAGGTGGGGGCGGGAGGGGGAGGAG
GAGAAGGAGGCTTGGCCAGGTGAGCGTCCTCGCCGCCGCCGGGCGTCCCAGCGTCAGCTCCGCAGCCCGGCACCC
GGGGGCGGAGGCAGGAGGCCCGGGCGGGGAGGTGAGGCTGAGCGCGGCCATGAGGAGGGGAGGAGGAGGCTGG
GTGTCACAGCTTCAGGCCACCTTGGCCGCCGCCTCGGCCCCAGTCCCTGGCAGCCGCCTCCGCACTCCGCCCCGCCC
TGGCTGCCTCTGTTACCCTCCAGGATTTTCGAGTTTCTCTTCTTAAAAAGAACTTCGTGCTGCGGGTTCGGCGG
174; SW vs SM; chr17: 67623575-67623903; 17; SOX9; PROMOTER
CGTGGGTCCGCCTACCCCAAGCACTTTTTGTGACGTCACAGCTCCGAGGACCATCCGGGGGCTTTACTCTCGCGGA
AGCCCAAAAGTTGGGGTTTGACCTCATGACCTTGGCCTTCCCGGGGGGCGGAGGCCCTCGGGACAGGACGCGCTGT
CCCCGGAGTCCGGAGCTCAGGCCAGTGGCAGTCGACCCAGCCCCCGAGACTCCCTCACGCCGCTCCAAAACCAAA
ACGGAGCCCAACACGAAGCTGGGTGAAGCCGTAGCTTGCAGGAGCCAGGGAGATGCGCTCTGCCCGGACTTCCCG
GGTCCTGTTGAGACGGAAAGGATCG
175; SW vs SM; chr17: 67627870-67631593; 17; SOX9; INSIDE
CGACTCGGCTGACGTTTTTGACCCGGCCAGGAGGCAAAGACCAAAACGTCAGAGCAGTAGCCCTGTTACTGAGGA
GCGTCGGCAGGGTCGCGGGTAGAGGGGGCTGGAGAATGACTTGTCAGAGCTCAGGTCGATGTGGCGCGGGGCGGC
CTCGAGAGCGCCGGGCTCCTGCGTGGCCACGGCCGCCGCTGCCAACCTTCGCGGGGACTTAGCTTTGCTTTCCATTG
ACTCCCTTTGCAAAAGCGCAGCAGAATCCTGACCAGCCGCACCAGCCCCGGCGAACCCGAGCATGTAATCTATTTA
TATGGATTATTACGGAGGAACAGCGGGCGTTGAGTCACCAAAACATTTGCTTCAAAAGACTATTTCTAAGCACTTT
TGCAGGCAGGCAGGCTCGCTCCAGGCGCGTAAACTCGGCTACGCATTAAGAAGCGGCTGCTTTTCGAATACTGCAA
ACCCAGCTAAGTCCCCGGTGCCGCGGAGAGAGCAGTGAAAAGAAATGTCGGAGGTGGGGGTAGATCCTAGTCTAG
ACACACACACTTGCGCGCACACACACACACACACACACAAGATTCGCGCGGAGAAGGCACTAAAATTCTGGCATT
CCGAGAGTACGACAAACTACACACTTGGAAGTCCCGGGTCCCCCGCCTTCCCCGCAGCACCCCCCGCCCCCCCACC
CTACCGTCCGCCCTTTGGCTGCGATCCCCTCCCCTCTCCTCCCCTCCCGCCTCGTCACCCAGCCCAGTGCCACAATCC
TCCTCCCTCCCCAAAATCGGGTCCAATCGCTGCCTGCCAACCCTGGGACTGCTGTGCTGTGATTGGCGGGTGGCTCT
AAGGTGAGGCGGAGTATTTATTAAAGAGACCCTGGGCTGGGAGTTGGAGAGCCGAAAGCGGAGCTCGAAACTGAC
TGGAAACTTCAGTGGCGCGGAGACTCGCCAGTTTCAACCCCGAAACTTTTCTTTGCAGGAGGAGAAGAGAAGGGGT
GCAAGCGCCCCCACTTTTGCTCTTTTTCCTCCCCTCCTCCTCCTCTCCAATTCGCCTCCCCCCACTTGGAGCGGGCAG
CTGTGAACTGGCCACCCCGCGCCTTCCTAAGTGCTCGCCGCGGTAGCCGGCCACGCGCCAGCTTCCCCGGGAGCCG
CTTGCTCCGCATCCGGGCAGCCGAGGGGAGAGGAGCCCGCGCCTCGAGTCCCCGAGCCGCCGCGGCTTCTCGCCTT
TCCCGGCCACCAGCCCCCTGCCCCGGGCCCGCGTATGAATCTCCTGGACCCCTTCATGAAGATGACGACGAGCAGG
AGAAGGGCCTGTCCGGCGCCCCCAGCCCCAC
199; SW vs SM; chr19: 42586101-42586705; 19; ZNF569; DOWNSTREAM
CGATTGGGGCCTCAGCCTTAGTTCTTCCAGCTGGACCCTGGGATGGTGCGGGGCTGTGGCTCTGGGAAGTATCTTG
GCCTGCACGCGGTTTACATCATTGGGAGCACAGGCCTCGCCCTCCGAACTAGAGGCCCAGAGCCCTTCGCGCCGAG
CGTTTCCCGCCTTTCCCCTGTCTCAAGTCATTGCCCAGAGGCCACTGCGGGCCGTTCCTGCCTGTCCGTGGAGCTGC
GGCCCCCGTGTTCCCAGGGTGAACGAGCACGTGCGAGGCGGTGGTGCTTGTATCCGGGATAACAGGTAGGGCGGC
TGCATGCTTCATAGAGGCCAGCCTGTGCCGACCGGGAGCTGTAGGACGGTCTGTGTCTGGGGCTGCTTATGGGTAT
TCGCGTGCGTCTGCCGGTGCGCGGTCCGAGCCTACTCACGAGAGCGTGTGTGTGTCTTCTGTCTCGTGTTGCTATGA
GTTTGCATCTGTGTGGCTGGAATAGCTTGTTTGTGGGGGCCCGCGCGTGACCTGTGTGTGCGTTACTGTGTGTGTCT
CAGGCAGGATAGTGACGGGCCGTGTGACTGTGACGCCATCATCAGGTTGGTGAGATGGCGTGACCGCG
204; SW vs SM; chr19: 57082579-57083180; 19; ZNF577; INSIDE
CAGCAGACCCCTTTTTAAGTACGCATGTGATAAGCAATGAACACGAACTGCCCAGAGCAGTCTCCAACACTGACAC
GATTCGCTTCCCCACCACGACGCCCTAGCGCTACTGTGCAACGAAGACCTCCAAGCACTGGTTCCAATGCGGAGAC
CATGGGCTCCCAGACTCTGGGAACTCCAACACGACTGCGAAACGAACTCCGAGCGAGGACTCCCCGAGAGCTCCC
CGCAACACGGACCTCACGCGCTAGCGAACAACAGAAAAAAAAAAGCGCGCTCTCCCTGCCCCTGAACATTCCCAG
AAGCCCACGCAGACCAGACCGATGACCTGTCTCCACTGCTGGAGGCGAGTCAGGGACCCGAAGTCTCTAAACACTC
GCCTCTACCCGCCGCCCCGCGAACCCCACACACTGCAGACGCGACACTCGCAAGTTTCGGGGATGGCGGCCGGCGA
GGGCATACTGCGTCTTTCCGGAGACACGGAATACGGCACCAGCCGTCCCTTTATGATGCAATATGTCTGCGCCCAG
GGGACGCTTGCTGGGAGCAGCCATTTTCAACCCTACTGCCGTAGAGCAGGCGGAGTCCCTCTTTTCGCG
233; SW vs SM; chr2: 119329502-119332035; 2; EN1; PROMOTER
CGAGGAGGCTGCAGGGACGCGCATGGAAGAGCCGGTGCGTGGGAGGGTTTGCGGGGGGGACATCGCGCCCCCTAG
GGGTGACCCCAGTGGGTCCCGTGTGCTCTCCGCGGAGCCGGCGGAGCCTTGTCTCTGCACCCGGCGCGCAGCGGCC
CCTTAAACAGTGGAACCGTGAGGCCGCTCTAAGCCGAAGGGCTGGAATCTGGGTTTCTCGGGTTTTATTTTAGACC
ATTCGGCACCAAGCCCGAGCTCCCCCGCCGCACCGCTTCCAGTCCCCTTTCTTTCCATAGAGCGACCGAAGCCGGC
GGTGGCGCAGGGAGCCGAGTCTGATGAGCTCGCGGGCGGCTGAAGGCCGGCTTCCCTGTGGGGAACGCGCCACCT
GTCGGCGCCAGTGAGAACTGCGTCTGTGTGGCGCCCTCGGGGTATTCGGGGCTGCGGGGAGATGTGTGCCTGAAGC
CCTCGCTTGCGGTGGGGACGTCCGGCCTCTTTCCTGGCAATTGACCCCTGAGGCGGGAGAGACAACGGAATTCCCA
CAAAGGGATCCTTCTCGGGATCTCCCCACCTCAAGACAGCTAAAGCTGGAGGAAAAGCCCCTCCGGGGGGTGGGG
GGTGCGGGTTTGCCCTGGATTCCGAAAGCAGAAAATACCCGAGCCACACAGGGACGGGCGCCGCGTTGGTAGTCG
GGGCTACGTTCCTACTCCCTCTACCTCCCCCGCGCTGTGTGACCCTGGGCGGAACCCCGCTGCTCTCTGGGCCTCAG
TGTTCTTATTCGTAAACTGAGGGCGTTGGAGAGATTGGTCCTCTCCCAACTCTGACCTTGAAACTGATACTGAATCT
GAGCAGCGTCTGTAGACACCTGTGCCTTGCCTTCTATTTCTAGCCTTGAATAAATCCTGGACTTTTATGTGCCATTTA
TATCCTAATCTCATATATATTTAATGTATAACTGCTGCCATATTGTTTTCTCAATTGTCTAGGTTTTCATTTGGATGG
GGTTAGGATGGTCCAAATTATCCCGATAAGTGCCCATTAACTTAAACCTTTTTAAAAAATGAAACCAGTAAAACTT
CATTCACTTTGCAGTGTGGACACTGCTGGAGAGCACCCATGTCGTGGGTCCGCGAGGACACAAGGAGGGGCTTAGA
GACATGCGGGAGGCTTAGATGAGAAGACAGCACCCGGGCAGCGGTCAGTGTTAGAGAGAGGACCCGTAAGAAGG
GCCGAGGCTAGAGGGAGAGCGAAGACTGAGCCAACGACGCACCTGAGCCCTGGGGTGGGGGTGGAGCGTGGCTCC
TAACCCAAATCTCCCTGCCAGGCAGTGTCCGAC
264; SW vs SM; chr20: 54633629-54640196; 20; TFAP2C; PROMOTER
ACCATGAGCTTCCAGTTCCTACGGGTGAGAAAAGAGGTGTCTGTGTGGAGGGCAGGTGCGACCCCGGGGGCTAGC
AGAGAGGAGAGGCTGCGGGAGGACTCGCTGCGAAGGGCGAGGGGTGGCGCTGGTGGGACGGGCGCCTGGGGCCG
TGCAGGGTGGCTCCTGGGTGCTGCCCGGGCTGCCTGTCGCCCAGTACTGGCGCAGGAAGACGGGTCCGCGCAGCGT
CTGGCAACAGTGGCCTGCTCCACGCCTGGAGGCGACCAAGTTCTGAGTTAAAGGCGCCGGGCTTAGCGGGAAGTGT
ATGGCGACCCAGAACACGGAGCGGAGAAGGCCTCAGGGGACACGAGGCTACCGCCTTAGTCCTCGGGCCTGCGCT
CCGAGCGGTTAGGGTGCGTACGGATGGGCTCCGGGATGTTAGTGGAGAGGTGACAGGAGTCTACCATCCCACGGC
CAAGCGGTGAGGCCTCCGGCGGGCGGGGGTCCCCCCCGGGGCAGGGCAGGGGGAGAGTCAGGCATGTCTCTAACG
GGCTCCCTAGTAGAACCGCTAGGAAAGTGGCCTTCAACCCTAGGCACCTTTGTGGCTTTCAGGTAGTTGGGCGACT
AAAAATTCACTTTTGCCTGCGTGAAATGGGACAGGTGGTTGTCAGGTTAGAGACTGTGCCCACGAAGTCTCGTTTG
GCGGGCCGGGACTGACGTGGACCCGGGCCCTCCTCGGAAGCTTGGGAACCTGTGAGCCCCTCAGCTCCTCGCCTCA
CTTTGCCTGTTTGAAAAAAGGAGTTGTTTGAGAGAAGAGTTAAGGCTCAAAAGGTTCTAAACGAAGAGCATGCGGC
CTGACGCGGAGCGCAAAGAAGCGAGCAGTGAACTGAATCTGGTGGGACTGAGACCAGAGGCGTCTGTATGCGGCG
GCCGCTTCGCGCCCTATTAAAGCAATCTGTTATTTACGATAATCATTTTGTAATTATTTTGGAGTGGCTGTGACTTGA
TGTCTGTGGTGCCCCAGGGCATGCCCTTACTGGACATAGATACTTCAGGGTGCCCTTTGACGCCAGTGTCGGTCCAG
TGGGTGCCAGTGCCGCGAAGGGAGGGTGGCGGGCGTGAATGCCCCCTCCTCCTGGGTTGCCGCAGGCCCCGGCCTT
TCCACCTTGCTTGATCCTGGCGGGTCTGCCACTTACTGAGGTCCCTACCGGCGCTCACGTTGTCCCGAGAGGCGGGA
CAAACGTCCGGCGCGTGGCGCGGAGTCTGCGAAAGCGCTCGGGGGCCTCCATCTTCTGCTCGGGAGTCCCTCCCTG
GGGCGGGAAGAGGAGTCGCAGATCTGCCCGAATCT
280; SW vs SM; chr3: 148619483-148620015; 3; ZIC1; DOWNSTREAM
GAGTGCGTAAAGGGAAATTTTTGCATTGGTGGATTTATTGAAAGCATGGAAACAGTCTTCGGGGGTAGGTGTAGGG
GGTGCAACAGCGAGACTTCCCCACAACACCTCCACCGAAGGCTGGGTCGTCGCGTTTGAGGGACCCTCCCGCCACC
CCGCAGCCTGGCGGGGCGCTTTCCAAATCGTCGCGGCAGGCTCTGGCCGGGCAGGGCGAGATGTGCGCATGCGCG
GCCTGTGGCCCGAGGTTCCACGTGCTGATGAATATGCATGAGACTCCCCCGCCCTCGCGGGCTAGGAGAGAACCAA
GCAGAGGCTCCGGCCTGTGAAAGTCGCTGGCTAGGCTCCCAAAATAGTGCGGCGGGGTAGCTGCACGTGTTTGTTT
TCAAACCGCGCTGGTTTAAGTGAGTTCACACTGCGGAGCCGACAGCAGAGGGTAGAGCAACCTGGGGCTGCTGCC
ATTGAACGGCGAGTTGAGCGGGCCTGTCAGGGCCTCTCCGCCGAGGGCCGGGGGCTGCATGGTGCTCGAGCCCCA
G
289; SW vs SM; chr4: 134288613-134289892; 4; PCDH10; PROMOTER
CGCGTTGCCCCTGCGCCTCCGGAAGGGGCACTCACAGCTGGTCAAGCGCGTCCTTCGGAACTCGGGGAGGCTCCGT
TGGGGCTTACACAGAGCTAAGCCCTGGGCCAGGGAGGATGGATGCAAGTACGTGCATGTGCAAACGTGTTCGCAG
ATACCCGCAGCGGTGCACACTCGGGCCCCTCGTGTGCACGGGCCCTGCCCACCACCCCGCCCGCGTTCGCCGCCGC
CCCACTGTCCTCATCCCGCGGGCTGACCCCAGGATTGGCAGAAATCAGTGGTCTAATCTAGCATGTGGGTAAAGGG
ATCGATCTCCAATTGCCAAAAAAAAAATAGAAAATAGCCGGTGTTCCCAGCCGCTGCGTGGCACTTGGCCGCACAA
GGCGCAGGGATATCAGAGGCGCTTTGCAGCGCTTGGCGGTGCTGTGTGCCGGATGCCCCAGAGCCCGGCGTTAGGA
TGCGGGCTTGTAGGAAGCGGCTCAAATCAACTCCTCAGTCACTGATGAACGCGCTCACGGCTCGAAGCTAGGTCCT
CGAGTGAGTTAATCTACTTGGTAAGTATTTTGTGAATATGTATTTTGGGAGGAAAGAAGCACGTCGAACGCGTCTG
CCCGTCTGTAAGCGCGTCATCCCCACATCCTTTAGCGTGAATTCCTCCGCTTTTTAGGAGTCACCTCCACCCCCTACT
CCGTATGCTTCGCGCCTTTCCTCACGGCCCCGTCAGACCCCCTCACCTTTAGATTGACACATGCCATATAATACTCA
CTTTTAAAATGCCCTGCCAGTCTCTCTAGGGTCCCTTAATTAGGTAACCCTTGCCAATTAGTGGGCCCTGCGCTACT
TTTTCCCTTCTCGATGATTTATTTCCATGATGCGTATTAGGGAAAATACCTTAAAGGAAAAAGAATGTTAGCATCAT
TGGAAATCCCGCTCCTAGGAGAGGATATCGCAGCTGTGCTATTATGTTTTCGCATTTGCTGATGCAAACAGGGGAA
CCTCTCTATTCCCTCCCCATTACCTGCGGCTGGGGACTGGGAACTTTCCGCGGCATTGTCCCCAGCCAGCGCCTCGG
GACCTGCGGGGCTGTTCTCGCTCTCACACTCACACTCACCCCGGCCGCCGGGCGAGAGTTCTGTCCCGCCCCGCCCG
GCTGCCGCGTGACGTGTCTGTTTGGCGGCCAGTTTGGCCAATCATCGGCAGCTCGGTGGGTGGTGCTCCTGGGCGA
TTGGTTGGCAGAATGAGGGCGCTGCGCAAAAACGGAGAAGCCGAGCGCTCG
290; SW vs SM; chr4: 134291113-134293078; 4; PCDH10; INSIDE
CGGAGCCAGACCTGACGGTGGAAATCTCTGAGAGCGCCACGCCAGGCACTCGCTTCCCCTTGGAGAGCGCATTCGA
CCCAGACGTGGGCACCAACTCCTTGCGCGACTACGAGATCACCCCCAACAGTACTTCTCCCTGGACGTGCAGACCC
AGGGGGATGGCAACCGATTCGCTGAGCTGGTGCTGGAGAAGCCACTGGACCGAGAGCAGCAAGCGGTGCACCGCT
ACGTGCTGACCGCGGTGGACGGAGGAGGTGGGGGAGGAGTAGGAGAAGGAGGGGGAGGTGGGGGGAGCAGGCC
TGCCCCCCCAGCAGCAGCGCACCGGCACGGCCCTACTCACCATCCGAGTGCTGGACTCCAATGACAATGTGCCCGC
TTTCGACCAACCCGTCTACACTGTGTCCCTACCAGAGAACTCTCCCCCAGGCACTCTCGTGATCCAGCTCAACGCCA
CCGCCCGGACGAGGGCCAGAACGGTGAGGTCGTGTACTCCTTCAGCAGCCACATTTCGCCCCGGGCGCGGGAGCTT
TTCGGACTCTCGCCGCGCACTGGCAGACTGGAGGTAAGCGGCGAGTTGGACTATGAAGAGAGCCCAGTGTACCAA
GTGTACGTGCAAGCCAAGACCTGGGCCCCAACGCCGTGCCTGCGCACTGCAAGGTGCTAGTGCGAGTACTGGATGC
TAATGACAACGCGCCAGAGATCAGCTTCAGCACCGTGAAGGAAGCGGTGAGTGAGGGCGCGGCGCCCGGCACTGT
GGTGGCCCTTTTCAGCGTGACTGACCGCGACCAGAGGAGAATGGGCAGGTGCAGTGCGAGCTACTGGGAGACGTG
CCTTTCCGCCTCAAGTCTTCCTTTAAGAATTACTACACCATCGTTACCGAAGCCCCCCTGGACCGAGAGGCGGGGG
ACTCCTACACCCTGACTGTAGTGGCTCGGGACCGGGGCGAGCCTGGCTCTCCACCAGTAAGTCGATCCAGGTACAA
GTGTCGGATGTGAACGACAACGCGCCGCGTTTCAGCCAGCCGGTCTACGACGTGTATGTGACTGAAAACAACGTGC
CTGGCGCCTACATCTACGCGGTGAGCGCCACCGACCGGGATGAGGGCGCCAACGCCCACTTGCCTACTCTATCCTC
GAGTGCCAGATCCAGGGCATGAGCGTCTTCACCTACGTTTCTATCAACTCTGAGAACGGCTACTTGTACGCCCTGCG
CTCCTTCGACTATGAGCAGCTGAAGGACTTCAGTTTTCAGGTGGAAGCCCGGGACGCTGGCAGCCCCCAGCGCTGG
CTGGTAACGCCACTGTCAACATCCTCATAGTGGATC
309; SW vs SM; chr5: 133887842-133891327; 5; PHF15; PROMOTER
CGAGAGAAGGTGAGCTCGGCGGGCGCAGCGGGTTGGGTGGGTGTGGCCCGCGGCTCCGGGAACAGACCCAAGGCC
CCGCCACCGCCGGCTAGCAGCCCGACAGCCCGCCCGCCTGGCGAGCGCAGGCCTTCTTCCAGAAACTGCGAGGATC
CGAGCGTACTGTTTGTGCACCACAAGGTCAAGTCGGGAAGTGGAGCCGGAGGAGGAGGGGCGAGGAGGCGAAGG
GTGGGGAGAGGGGCGCCCCGCTTCCTCGCCATGTGCCAGCCTTTGGAAGTTACCCCTCGGTTTTTAACTTTCGGGGT
AATGGGAAGGAAGAGCAAGGAGGAAGGGGTCGCCCCGGGGCAAAATCCTGCTGGGGCCAGGATGTGCAACCACC
CACCCACCCGCTCATCACTGCCTTCTCCACCCCCCGGAAATGCGGGGCAGCGCTAGGCGCCAGGAGGCACCCGGGA
GGGGGAGGGGGCGACGAGCCCAATGGGGACCCTCTCTACGGACCGCCCCTCTTTTCTTGAGGTCGCCTACAATTTC
AAGTTCCCCCGCCCGGAATGTTAGTTTTCCATTTGGAGTGGGTGCGGGGAGGAGGGCCGCGCCCCCTCCCCAAGAC
TCGCGTTCGCCCCTCCACCCCCTCCTGCCAGCACTTTCTGCTCACTTCTGGTGCTGCAAAACGCCACGCAATTTATTC
GATTCGCATTTTAAAACCAGCGATTCAGAAGCAGGGCCTAGGGCGAGGGTCTTCCAGCGCTGGAGCCCCGGGGGG
CGGGGTGCTCAGTGCAAACCTCGTTCCCTCGCGAGTGTCGCCTCCAGGCTGTTATTTGCAAAGAAACGTCTTTTTGA
CGCAGGGAGAAATGGCAAATTTTAAGTACGTCATTAATATGGCGCCAAAAGATTTTTTTAAGTATAAGCTCGCTTTT
TTTTTTTTTTAAGGTTGCGGGGGGCGCCGCCCGGGTCTGGGCGCGTAGGGGGCGGGGTGTGAGCAGAAAGTGTGAG
TGAAAGAGTGGAGGGGGGGGGTATGTGTGTGAGTGTGTGAAGTGTGAGCAGACCTGATGGGACTTGCACGGGCGC
AGCCGCCGCTCGGGCCCGGCCCTGGGGACAGGGCGGGCTAGGGGCGCCCCAGAGTCATGGGGAGTCCGGGCCCAG
GGTGCCAGCAGGCGTGGTGGTGGGGCTGCGAGGGAGGGCACCCTTCCCCCACGGGGCCCGCAACGCTACCTGGAC
TCCCCGCCGGAGCCAAACAACTGGGCGGGGGGTTGGGGGGGCGGCGACGGGGGTGTCGGGAGCGGAGATCGAGT
GAATAAGAAAAAAGTGGCTACTCCCCCTCCCTCGCTCC
328; SW vs SM; chr6: 5080084-5080849; 6; LYRM4; INSIDE
ATCCCTATTCCTTCTCCTTCCTCTCCCTTGGTAACGGCACTCCTAGGGCTGTGGAGGGTGCGGTTCACTCCTGGGACT
GTGGAGGGTGCGGATCACTCCCGGGACTGTGGAGGGTGCGGATCGCTCCGGGGCTGTGGAGGGTGCGGATCGCTC
CCGGGGCTGTGGAGGGTGCGGATCGCTCCCGGGGCTGTGGAGGGTGCGGATCGCTCCCGGGACTGTGGAGGGTGC
GGTTCACTCCCGGGACTGTGGAGGGTGCGGATCGCTCCCGGGACTGTGGAGGGTGCGGATCGCTCCGGGGCTGTGG
AGGGTGCGGTTCTCTCCCGGGGCTGTGGAGGGTGCGGTTCACTCCCGGGGCTGTGGAGGGTGCGGTTCACTCCCGG
GGCTGTGGAGGGTGCGGTTCACTCCCGGGACTGTGGAGGGTGCGGATCGCTCCTGGGGCTGTGGAGGGTGCGGTTC
ATCCCGGGACTGTGGAGGGTGCGGATCGCTCCCGGGACTGTGGAGGGTGCGGATCGCTCCCGGGGCTGTGGAGGG
TGCGGTTCTCTCCCGGGGCTGTGGAGGGTGCGGTTCACTCCCGGGGCTGTGGAGGGTGCGGTTCACTCCCGGGACT
GTGGAGGGTGCGGATGCTCCTGGGGCTGTGGAGGGTGCGGTTCACTCCCGGGACTGTGGAGGGTGCGGATCGCTCC
TGGGGCTGTGGAGGGTGCGGATCGCTCCAGGTGTGATTTTCATGGGAATGGAACTGGCTCCCTAGGTGGTGTTCAT
TTC
379; SW vs SM; chr8: 65653638-65653908; 8; BHLHB5; PROMOTER
CGAACGCCGGGGGAGGGACGGTGGGAGGGGTGCGGTGAGCGAGGGGCTGCCCTGCATGTAAACAGCCTCGGGGTC
CCAGACGAGACAAGTGGGACCGCTCAAGCCTGCAGAACCCCCGGTCTCGGTCCAGGCACAAGAGCCTCCCGTAGC
CCCGCCTGATCTTTCTCAGTCTTTAGGTTCCAGGACATCCAAAGGAGCGTAGGTAACCGACGTCTACGTAGTATTTC
TGTCCCCGGAAGACTGGATCTCAGGGAATTATTATGGAAGATA
386; SW vs SM; chr8: 72916429-72917309; 8; MSC; INSIDE
CGGGGCAAGGGCGCGTTCCCTATCGCAGGATCACTTGCTATGGTAAGCCGCCCACCCTGCGCGCTCCTCCGCGCGG
GGAAGAACCTGCGCGGCAGGACGTGGTGTTGGAGTTGGGGCGCCCGGAGCGGGAGTGGGGAGACCTGATGCAGA
GAGTCTGGAGTCGGAGCTGGGGGTGCTGCAGGTAGGAGCAGGAGCGGGGCGGAGAGGGAGGCCCGAAGAAGACC
CCACACAGGTTGGCGCAGCGGGGCTTGGGGAGGCTTCAGCCCAGAAGTGGAGAGGGTTGACAGACGCCGCCTGAT
TAGAAAAAGCCGGGAGCTTGGGAAGGAGACGGGATTGAAGAAGCCACCCGGCAGGGAGGCCGAACGGCCCAGAG
CTCTCCGGGTAAAACCCGCCTGCGGTGATCTGGGAAGTGTGTCTCCACCTAGCCCTGCGAGCAGCGGCCTTCCTCCC
GCCCGTTGAAGGGCGCTGTGCTGGAGTACGAACCCGGCCCAGAGAAGCCACTCGCCCTTCTTTGTCACTTAAAACC
CTGTCCCGACGCGGATCTCACGTCTAGACCTCTGTCTTTAAAGCGGATGTAGAGCGCGTTCTAACCGTTCCCTAACC
ATTGTGTCACCGCGAAAGGCGGGGCTGTGTGGAACCGTCCCGCACGTGTGCGATGAATCTGGCTCCGCTGAGAACG
GATCCGTGGGCTGTCTGGCGCGGGCTGGGGCAGCAGCCGAGAGTTAGTCTACAGAGCTAGGGCCCGAGGGTGGAC
CTGCGTCCGCGTCTCGTCACGAAAGGAAGCTCTTTTGGAGAGGCAAAACGTGGTCGCCCAGATCCGGCGCAGCTGT
AGCCGTGGGCGCTGTGCAGTGACAGCCACACCCGCCACCTGTCACG
400; SW vs SM; chrX: 2845926-2846236; X; ARSD; INSIDE
TTTCTCATCAGGATACAGTTCCAGCGTCGCACAAACCCGAAGGAGGAGTACCAAGAGATGAAAAACAGGCAGCCC
ACGCCGGCCATGCCGGTGACTGCTCTCGCGGAGACAGAGAAGAAACCGCAGGCTGGCCGGCAGCCAGGGTGAGAA
TCCCCAGCGCCAGGAACTGGGTGTAACCCCAGAGCTGCGCCCTCAGGGCGGCGTCCACTTCGGGGGGCCTGCCTGG
GTCACAGTCGTTTGTGAGCGTGAAGGGCATGCCGTAGAAATAGTCAAATCCGTGGTTCAGGGGGTGTGGCAGTGAT
CCCCGCG

TABLE 5
CpG islands potentially involved in progression and death of disease. Provided
for each are: ID; Comparision; Coordinates of selected regions containing relevant CpG
islands based on Homo sapiens full genome as provided by UCSC (hg18, March 2006);
Chromosome; GeneName; Location relative to gene Sequence.

406; DOD/alive; chr2: 115635091-115637285; 2; DPP10; PROMOTER
AAAAAAGAAATGAGTATCAGAGTCTTATCATCATTACAGTTCTTATGGTTTTTAACCGACCTTTAGGAGTTCCACCG
ACGTGACCCAAGCAGCTGGTAGGAGAATCTGGGCTGGATTCGGAAGGCGCGCTACAGGACGCCCGCTGCCCCGGA
TCGGAAGGCGCAGCACCTCAGGCCACAATCTCAGACACGCCCTCGGGTCCCGACAGGTGTTGCCCGCCTCCCACGG
GGCTCCGGGACCCCGGTCCCCGGGCTGCCCCCTGCCTTCTGGGGCGCTGGAGCGCGCGCAGTGGCGGAGCTGAGCC
ACCCAGTGCTTCGCACGGGCGCGCTCCGCTTCTCCGGGTTTTAGCGGAAGCCTGCGGGGGGCGGGGTAACCGCGGA
AGCCGGCGGCCGTGGGCGCGCGGGTTGGGGGCTCTCGCGCCGCTCCGGGCTCTCCCCCCCCCCGGCTGCGGTTGCC
GAAGAGAGGCCGGAGGCGGCGCCCGGCTGCTCCACCTGGCGCGCTGAGCACCGCGCGGGGAGCCCCCGGGGGCGG
GCGGTGGGGGGCGCGGACTGCGGGAAGAAGGCACGAGGCGTCGCCGCAGCTCGGTCAGGGGCCGGGGCCCCGCC
GCTCGCCGCTGCACTAACTTGCCGCTTGGTCTCGCCTCCCGCCGCGGCTCGCTGCGCTTTGGGTGGCGGGGGGCGG
GGAGAGCGGGGAGTCAGAGGGTCTGCGGTGGCCGAGGGAGGGACCCTACGACGGGGAGCCCGCCCGGTGCCGCTC
TTCTTCCCCTCCCCGCCCCTCCGCTCCCCCCACCCCGTCCCTTCCGCCGATTCCGGGAGGACGGGCGCCCGTGACCT
GCGAACGCTGCCAAGTGACGGTCCCCGAGTCTGAAGCGCCCGCGAGGAAGCGAGCGCCAGCGCGGGCCGCCGGCG
ATGACGGCCGCGAAGCAGGAGCCGCAGCCCACCCCGGGGGCCAGGGCGAGCCAGGCGCAGCCGGCGGACCAGGT
GAGAGCGGCAGCCGCGGCCAGGCCCTCCCGGGAGGGGTGGCTCCAGTGCGCGCTCCGCCCGCCTCCCGCTTCCCAG
GCTGGGCTCCCGCGCCTCCCTCTTCTCACCCTCCCCCGCCCCGCCCCAGTTCCAGGCTCTCCTGCTTCTCCACGGACT
CTGCGGGAAGTTAGAGCCTCTGCTGCGCTCCGGGGCCCGGCGAGAGGATGCGCAAGGTGGAGAGCCGCGGGGA
407; DOD/alive; chr3: 62330280-62330608; 3; FEZF2; INSIDE
AAAATTGGTGCATTTAGGAAGGAAATGCTATAAACGCCTAATTCCTTCTCGTTCTTTGGGGTTGCTGAGCTGACCTC
GCCGCGAGGGGAGAGGGGGAAAAAAACGCGCACTAATGATTCTGTTTATTAGTTATATATGTATATATTCCGTGTT
CGCTTGTACAGGAGGATTTACATGGCTGTATAAAGATGGCTAGGGGCGCCGCGCTCTTCTGGGGCGCTCACGGTGA
CAGGCTGGGGTTAAAACTGGCTGCCCCAGGAGAAGCGGAGGCCTGGAATTAAATACGTTTCGGCGCACTGATTTAA
ATAAGTTTCCTGAATATACAAA
408; DOD/alive; chr3: 62331767-62332398; 3; FEZF2; INSIDE
AGAGCTTCCCCCTACACCAGGCACAACCCGATTCTAAAGAAATGCTGCGCCCGCGCCTGCAGATTTCGCCTTCTCTC
TCCAGATCCCTCTTTGCCTTCCTGCCAGCCAGCCGCGACCCGAGTCCTGAGTGAGGTGAGAAGAGAAGGCCTCGCC
TGGCACGTTACCTTTTTGGTGAAAGCCTTTGCCGCAAAATTCGCAGACGAAGGGCTTGTAGCCCGCGTGGATGCGG
ATATGCGTGTTGAGCGTGGAGCTGCGGTTGAACGCTTTGCCGCACTGGTTGCATTTATGTGGCTTTTCCTAGGAAAG
GGGCGAGTGCACAGTAAGGAGAGGCCACCTCGGGAACACCTGGAAGGGACATCCCCCCCACCCCCACTCAACCCT
AGAGGGTAAGGGATAATCTTTGGCCATCGTATCCCCGGTGTTATCACTAAGATTCTGGGGGTTGCGCGCGGGCTAG
GCCCGACCCGGGGGCACGTACCTGGGTGTGGATAATTTTGTGCCTGCAGAGCGTGCTGGCCTGGCGAAAGCCTTTG
CCGCAGACTTTGCACACGAACGGTCTGGCTCCGGTGTGGACCGGCATGTGGCGGGTGAGATTATAGTGAGCGTTAA
ACACCTATGGAAAGACATGG
409; DOD/alive; chr20: 22514762-22515148 20; chr20: 22514762-22515148; Unknown
ATCTTTTGCTGCTGAAATGCAGATGGTTTGGACACTTCATAAAGAGGGCATTAATAAGCCCGCCGAGTGAGCGCCA
GGGCGGGATTCTCCCACAAATTCAAGGTCTGTATCTCCGCGGAAGCCGTGCTATCGGTCCCCCGAGTGCTGAAGGC
TCCCACGAGGGGCAGGCCAGCCCGGTGCGCCAGGGAAGGAGCTTGATGGTGTGTACGGCTACGTAGTGAACGTGG
AGGTGTGCGCAGCGGAGTCAGAGGGGCTAGATCCCCAGCATTTCCCACGGGGTCCCACCTAGCGCGTGGATGGAG
CAACTTCATCACTCCACCGAGGGCAGGGAAAGAGGAGTAAGCATTGGAGCTCAAAGAATGAGCAATTTTTCAAAG
CTTTCTCC
410; DOD/alive; chr20: 22510737-22514165; 20; FOXA2; PROMOTER
CGGGTGGTTGAAGGCGTAGTGGTGTTCCGGCTTCAGGTGGGCCTCAGGCGGCAGGCCCGGGTGGTGGGGGGGGCC
CAGCAGGTGGGCCGCGGCCTGCTGCTGCTGCCCGGGAGAGGGCGCCGGCTCTGGGGGCTCAGCGCCGCAGCCGGC
GTCCCCTTCAGCTCTCCCAGGCCCCCTCGCTTGTGCTCCTGGCACGGGGAGGCGCTCGAGTGAGGCGACTCGGTGC
CCGCCGGAGTCTCGGAGGCCGGCCCGGCGGCCTCCCCGAGTTGAGCCTGTGAGGCCTGGGCTCCGGCGGCCGCTTC
TTGCCGCTGCCGGCGGCGCCTGCGGCCTCCTTCAGCGCCAGCTGCTTCTCGCACTTGAAGCGCTTCTGGCGGCGCAG
GTAGCAGCCGTTCTCGAACATGTTGCCCGAGTCAGGGTGCAGGGTCCAGAAGGAGCCCTTGCCGGGCTTGTCGGGC
GAGCGGGGCACCTTCAGAAACAGTCGTTGAAGGAGAGCGAGTGGCGGATGGAGTTCTGCCAGCGCTGCTGGTTCT
GCCGGTAGAAGGGGAAGAGGTCCATGATCCACTGGTAGATCTCGCTCAGCGTCAGCATCTTGTTGGGGCTCTGCTG
GATGGCCATGGTGATGAGCGAGATGTACGAGTAGGGCGCTTTGCGTGCGTGTAGCTGCGCCTGTAGGTCTTGGGGT
CGCGGGCGCGGCTCAGGCCCGCCTGCCCGTACATGGGGCTCATGGAGTTCATGTTGGCGTAGGGGGCCAGGCCGCC
CATGGCCCCGGCCGCCTGCCCCCCGAGCGGGCTCAGGCTGGGACTCAAGTGCGGCCCATGCCCGCCACGCCGGCCG
CCCCGGCCGAGCCGCCCATGCCCGCCATGGCGCCCGCGCCGGGGGACATCCCCGCCAGGGACGGGCTCATGCCAG
CGCCCACGTACGACGACATGTTCATGGAGCCCGCGCTCATGTTGCCCGAGCCGCTGCCCATGGCGGCCGCCGACAT
GCCATGTACGTGTTCATGCCGTTCATCCCCAGGCCGGCGTTCATGTTGCTCACGGAGGAGTAGCCCTGCGGACAGA
GCCCCGGGAGGGAGGCGACAGCGTTAGCACCGCGGCTGGAGGGTGCCCAGACCTCCCACCCACCGCCCAGGCCTC
CGCGTCCGGGGAGGCCTCCGGGGGCCCTTTCGTCCCCGATGGCCCAGTCTCCGGACTCCGAGTCTGTTTCATT
411; DOD/alive; chr8: 57520625-57522095; 8; PENK; PROMOTER
ACAGAGGACTTCTCGGGGTTCCCGAATTCCCAGGGTTAAAACAAACTTGGTTTGTGCGGCGACACCGCTACCCTTA
CCGGTCCCCAGATAGTCGCGGATCCCCGAAGCTAAGAGCCAGGGCAGGACTCAGTCAGTGTTCCAGGAACAGCTG
AGACACCCAGGTCACCACGGGCTCGCGCCGCTGCGGCTCCGACCCGGTGCGAACCGCCATACGCGCCGCGCGGTG
GGCCGGAGGCAGTCCGCGTACTGTTGCGGAAACTCTGTCTCACAAGGTGCGCAACACTCGCCGCGCGCAACACCAC
CAGGAAGTTGATGTCGGCCGGGCGCACTAGGCGGTAGCTGCACGTCGCGCAATCCTGGCTGCATTCGGCCCGCACG
GTCGCCAGGAGCCCGGGGCCGAGCAACAGCAGCCAAGTGCAAAGTGTCAGGAACCGCGCCATGGACTGCGAGGA
GAGAGGGACGCGTGCTTCGGCCTGCCTGGGCGCAGAACGGGGTCCCTCGGCAGGACCCTCGCCGCGACAGCCTCA
GCAGGGGATCGTCGAGCAAAAGCCCGCAGGAATGCTCCTTTCTGGGGCCCCGCCCTCCCGGCCGACAGCTTTTAGG
TAGACGTGGAGGCGACTCAGATCGCCTCGCGGTTCCCGGGTGGCGCGGTCGCCCCCAACGCGAGGCTGCCTGGGGC
ACCCGGCTCTTTTCCTGGGCGTCCGCGGCCCAGGGCAAGATTCCGGAGAGAACGCCGCAGACCAGGGGCGAGAGA
GCGGGAGCCGGGGGAGGCAGGACATTGACTGGGGTCACAAAGAAGAACCAGGGAACGCTGTGGGGAACCCATGC
AATGAAATGTGAAGGGAAAGAGGGCGGGCAGACGTCCCCAAACCCGCTCAGCAAAGACAAGCTTCGGGCAAATTC
ACTCACGTTGACGCTGTTCGGATGGAGCAGGCCAATTGGAAAAGCCGGGTTCAGACACGACTCTAGAGGGAAGAG
AAGAAGGCAGTGTGAGGAGGGCGGGCGTCGGGGAGAAGCGGGCCCGGGTGTGCTGGCGGACGTAGGGCCTCACC
GTCGCGGTCCTCAGCGTCTCTGCGGGGTCACGGGCCAGGCTGCGGGGCTCTGAGCGCCTGCCTCGCCGTCCCGGGG
CTTAACGGCTGCTGGAGCCACTTTATAATTACCCCAAACCGAAGGAGGCGCGCGCGCCCCAATCGCCGGCGGGCTG
CAGCT
412; DOD/alive; chr17: 24303411-24303889; 17; PHF12-SEZ6; DIVERGENT_PROMOTER
GATCTGCTCTTCAAGAGGAAGCCTAAAGTGGAGAGTTTGTAATCCTGACAACGCCCAGCTACCTGCGTACCCCGGA
CTCCAACTCCCAGTTTGCAACGCCAAAGGGTCTAAAGGGCGAACGGGGGCGATTGCATCCTGGGAGCAAGAATCG
CGATAGGTCTGATGGATTGTGGGAACTGTAGTTCCATGTGCGTGCGATGCACTCTATGGTAGGATAGACCCGGGGA
AGAGTCATTTGCCGGCCAGGAAGGGATACTTTTGAGAGCGTCAGTGCTGCATGCCGGGATTTGTAGTCTGAGGCCC
GCCTCGCATTCTGGGCGGGGATTCATGCTCCTTTTCAGGGCGCCCGCTCCTCCCCTTGGTATTGCCGTCAGAGATGG
GCGGTTGGTTATTGCTGCCTGATGGTGGATGAAGCAGTGGAAAAGAGAATTGTATCGTTGTGCTTGACACTAGGGG
TTTAGTTGGAATTGTGCTTC
413; DOD/alive; chr1: 149638545-149639227; 1; PSMB4; PROMOTER
TAGGATTACTGCATGTTCAGCTACGACCTCATCAACATGGCTGCTGGAAGTCGTCTACAGGAAAAAGAAAGAAATG
ACACTGAAGGATCACTTCCGCTTCCGTTGGCGCAAGCGCTTTCATTTTTTCGCTACCGTGACTAAGATGGAAGCGTT
TTTGGGGTCGCGGTCCGGACTTTGGGCGGGGGGTCCGGCCCCAGGACAGTTTTACCGCATTCCGTCCACTCCCGATT
CCTTCATGGATCCGGCGTCTGCACTTTACAGAGGTCCAATCACGCGGACCCAGTAAGTTCTCGGCGCTTCGTTTGCG
TAGCGGGAGGGACCGTGGGGCCTGGTGCTGCCGGCTGGTTTTGAGAGCCCGGGAAGGTGAGGCGGGGACCCCGGG
GGCGCGCGAACGGCAGGGGAGCTCAGGGCGCGGAGTCCTGGAGAATGCAGAATAATTGGAAGGAATTATAGAAA
ATCAGAAGCGCAGCTAGTGCGCGGAAAGAGGGCGCAGTCCATCCCCCCTCTCAGCTCCCACCGTTCTCACTCTTTA
GGAACCCCATGGTGACCGGGACCTCAGTCCTCGGCGTTAAGTTCGAGGGCGGAGTGGTGATTGCCGCAGACATGCT
GGGATCCTACGGCTCCTTGGCTCGTTTCCGCAACACTCTCGCATTATGCGAGTCAACAACAGTACCATGCT
414; DOD/alive; chr21: 36990008-36995832; 21; SIM2; PROMOTER
TCCTTAGTGACAAGGTCTCTGATCTCCTGCTGCCACTGCAATAGCCTCTCCCATCCCGCGGGGAACGGCCGGAGTTC
TTCCCTTGATCTCTCCCGAGTCGGCTTCCGCTGGGGATGGATCGCAGGTAGCGCCGGCGCGGCCTGGGGAAGAACA
GTTGCGGAGCATCTGAAGCGGAAAATCCAAGCAGATGTGAGGCGATCCGGGCCCGCCTCGTTCCTCTTGGGGCCTG
AATTTCTTCCAGATAAGTTTCCTAATGGAACATTTCTAAGAGGTGGGGTACGAGGCGGCTTGCTCGCACGGCAGTG
GGACAGACTGCGGGTGGGGACGTACTGAGAGGTCCGGACCTCAATGCGTCCGACCCGTCTCCACACCGCCCTTTTC
CAGCCCCCAGTCTCCTTTCATTCCCTACTCTTCAGGCTCCTTTGGGGCCAGTGGGTGAACCGCCATTTAGAACGGTG
CCTCGGACTCGGGGTCGTGCGCTCCATCTCTGCCTCCCCCCTGGGGCCCGCGAGGCTGGTCCGGGCTTTCTGAGCTG
GGCGTTCGGCTTTAGGCCCAATACCTGGACCAGGAATTTCTTCTCCCCGCGCCAGAAGGGAAAGACATAGGAGGTG
TCCCAATCTGCGGTCACCGCCGATGCTCCTGACACTCTAGTGAGCACCTGCCCGGTACTTTTCCATTCCAACAGAGC
TTCCAGCTTCATACTAACTATCCCACATACGGCCTGTGGGTATTAGCTCTAAGTGTCCTTTTCCGAGGGCCCGAGGC
TCCCCCTCCAGCAGGGAGAGCTCCGGGACGGCCCCCACCAAGGGTTGGGTTCTTCCTTCACAATTCCACAGAGGCA
TCCCTGTCCTTCCTACCTGGGAAACCTCGAGGTGCGGTGCCCGTGTACTTCTGGTACTTTGCGTGGTGCCATCAGGG
ACCCCAGAGCCACAGCTGCGTGTGTGTGTGGATGTGTGTGTGTGTGTGCGCGCGCGCGCGTGTACGGCGAAGGATG
TGCTTGGGGGAGCCGAGTACACAACGTCTGCTTGGGCAGCTGCTGGGCAGGCGTTGGGCCTGGAGGTATCTCACAC
CCACGTATCTTCCAGTCTTCAAACACGGCATTGCTCTGCCTCCCGTAGCGCGCTTCGAACCTGCCTCGCGGACACGT
GAACAGAGGCTGCCCTGGGAAGATAAGTGCGCTTTCCCGTAAAATCCGGGAAATTTGCCTTG
415; DOD/alive; chr1: 232106869-232108183; 1; SLC35F3; PROMOTER
AAGTACTGAACTGGGAGCGGCAGGGTTTGAGTCAAAAGAGAAGGGTGGAGGAGCGCGTCCGGTGCCCCAAGGCTT
CCAGGCGGAGCGCGGGCGCGAGGTGCGGGCTCCGGGGCGGAGCGCACGCCCGGAGGCGGGAAGTGGGCAGCAGC
GCCCCCGCCTGGCCCCTGTGCGCCCCGACCCGACGCCTCCCCGCCCGACCAGGTGCCTCGAGAGCGCACAGCTGGG
AGGGCTCTCCCGGTCGCGGCGAGACTCACGCCTGCGGTGTGCTAGGGCGGCGGCGACGGTGACGGGCGTGGGGCC
GGCGCTGCCTCGGCTGCCGGGTCGTTGCGGCGAGCGCGCGGGCCGGCCTGGAGTCACCGGGCTGAACCGCCGCGC
CTGCATCGTGCCGCACGCCGCGGAGGCGCTCGGGTACAGACCGCGCGGGCGCGCACAAAGCGGCCCGGGGCGGCC
GGCGCGGCGCAGACCCTCGGTGGCAGCGCACTCCAGTCTTCCCAGGCTAGCGGCTGCAGGGAGCTCCGGCCCGCG
GCCCCTCCGCCTCAAGTCTGGGAGCTGCCGGTCCCACTCTGTCTTTGCCTATGGGGATTCGAGAGTTTCCCAGCGGC
GCACCCAGGGGCAAGAGCATTGCCGTGTGAGTAGCGCCCCGGCGTGGGTGAGCGAGCCGGCGGGCGGGAGGCCGG
AGCGCCGGGGTAGCCCTTTGCAGCTGGGACACTGGGCAGTCTGAGGCTCGGGGAAGGGCGGCGCGCGCGGTGGAG
CTGCTCGGGAAGTTTCAGTCATTCTTTCCCTCGCCACTCGCCCCAACCCGCACTGCCCCGAGAGCCCAGGATAGGGC
ACTGGGGACAGCTGCCCGGGGAAGTGGGTGAGTGGCGGCGGGAAGGGAGGAGCGCGGGCTCTGCCGCGGCGCTC
GCCCCCGGAGGGCCCCGGCCGCGCTCTTGCTCTCGCCCTGGGATCTGCTCCTCACGAATCCCCTCCTCTCTGTCCAT
GCTCCCCTGCCCACCCACCTGCCCGTGGCGCCTGCGACCGCAGTGGCATGAGGAGGTCACCGGACGTCAGCCCCCG
GAGACTGTCCGACATCAGCCCCCAGCTCCGGCAGCTCAAGTACTTGGTGGTGGACGAGGCGATTAAGGAGGATCTG
AAATGGTCGCGCTCCGTGGAGGATCTCCCAGCGGGCCGGTGGGGCTCACGTCCATCGAGGAGCGGATCCTGCGCAT
C
416; DOD/alive; chr6: 21704622-21705099; 6; SOX4; INSIDE
TGCATCACCACCTTGGTTTTGTTTTATTTTGCTTCTTGGTCAAGAAAGGAGGGGAGAACCCAGCGCACCCCTCCCCC
CCTTTTTTTAAACGCGTGATGAAGACAGAAGGCTCCGGGGTGACGAATTTGCCGATGGCAGATGTTTTGGGGGAAC
GCCGGGACTGAGAGACTCCACGCAGGCGAATTCCCGTTTGGGGCTTTTTTTTCCTCCCTCTTTTCCCCTTGCCCCCTC
TGCAGCCGGAGGAGGAGATGTTGAGGGGAGGAGGCCAGCCAGTGTGACCGGCGCTAGGAAATGACCCGGAACCC
CGTTGGAAGCGCAGCAGCGGGAGCTAGGGGCGGGGGCGGAGGAGGACACGAACTGGAAGGGGGTTCACGGTCAA
ACTGAAATGGATTTGCACGTTGGGGAGCTGGCGGCGGCGGCTGCTGGGCCTCCGCCTTCTTTTCTACGTGAAATCA
GTGAGGTGAGACTTCCAGAC
417; p-np; chr1: 160220156-160220709; 1; OLFML2B; INSIDE
TTGGGGTTGTAGTCTATCTGGGTCGTATAGGAATACTCATTCTCGAACAGCAGCCTGGGGACGATCTGTGTGTTGGT
GTGGGTGTCGAAAGCGTAGGAGATGTTGGCATTCCGCTGGTTGTAGCTATCACGGCATACAGCACCCCACAGATGA
CGAAGCAGTTGCCGTAGAAATTCCTCCGGAGCCCCGTGCGCCATGTGGTCTCCTTCTGTGTGCTCAGGTCCGCGGCA
TTGAGCTTGCTCAGGACAATGACCTCCTGGCTGAAGCCCTCATCGTCCAGGGCCGGGTAGATGAGCCATGGCCATT
CTCGTCCACAGCAAAGTCCACGTCTGAGTGGCCCTGCCATCGCCAGGGGGTGGCCTCCTCGTAGGCCACGTCATGC
AGCATGGCCCAGGCAGCCACGTAGCGCTGCTTCAGGTCGTACTTGATGATGTTGCGGGTGAAGGCGCGATTGTAGT
AGAAGGCGCCATTTATACCACGTGGCCTGTGCCGATCCAGCTGTACGGGAGCTTGTAGGAATTGCTCCAGCGACCT
GCAGGTGGGGAGAAAAC
418; p-np; chr3: 129688131-129694023; 3; GATA2; INSIDE
TCACAGGCCAGCTGGAAGTGGGCAGAAACCCTGTGGGTCCCAGACCCTCCCCAATCGGCCGCTGCTCCCACCTCTC
CCGCCCCAATTTTTCAGCAGCTCGATTCCTGCGGATCCTACATCCGGGAAGAAGCAGACGGGCCCTCCTCCCCTCCC
TCGCCTGGCGCGCGGCGCCTGGGTTCTCATCACCACGGGCCCAGTGCTCACCGTGCGCGGGGCTGTAGGAGACGCG
CGCCCGCGCGTGAGCGGGGTTGGCATAGTAGGGGTTGCCCTGCGAGTCGAGGTGATTGAAGAAGACGTCCCCTCGT
CTGGAGGCAGCAGCTGCGCGGGTTCCATGTAGTTGTGCGCCAGGCCCGGGTGGTGTGAGTCGGGGTGCTGCGCATT
CAGCACGGCCGGGTGCGCCATCCAGCGCGGCTGCTCGGGCGCCACCTCCATGGCCGGCGGCGGCGGCTCAGGGTCT
GGGTGCAGACGGCACGGCCCTGCGCGAGGAAGGGGGAGTGAGGCGTGCCGCCAGCGCCTGACACCCCCCAAAGTC
CCACCACGAGGTGTCCCGCACGCCACGGAGCCCCAGCCCAGATCCGGCGAGAAAGAGCACCAGTCCCGGGTGGGA
GGAAAGCCCAAGGCTCAAAACGAAAGGAAGGCGGGGAGGGGGTTCAGCCACGCACACTCACGTGGTGACCCGCG
GCTCCAGAATCACACACCCGTGCACATGGGGTCACGCCCGGGGACGGGTCCCGACACCAGTGACCCCAACAAACG
CACAGAGCAGCACTTCAGTCAGACACTCACACTGAGCCCCCCCGCCCGGTAGACAAACAATGAACACAGACTCAA
AAGTTGGAGACAGGCGCCCGGGCACCCAGTGTGGCACTTGATCCCAGCGACACGCACACACCCACACTTGGCGCC
AGATACACATACTGATCTCAACCCCGAAAACATGCACACGCAGCCCCCTGAGCGCAGTACTAAGCGGCACAATCA
GGACCTTCAACAAAGCACACCAAAGCAGTCGCCCGCAGCCTGGCCCCCCGCCCTAAGTCCCCCCAGAGTCCCCTCA
AAGCTAGGAGCGCCCCAGGCCCCCAGCCGGCTCTCAAACCCCAAACTTACACACGCAGCCGTGGGGAGGGGAGGG
ACTCGGCCTCTGAGAGTGAAGGAGTTCGGCGGGAGCCCCGAGGGCGACGGGCCCAGGGACAGCACGTCCGGAGGC
TG
419; p-np; chr6: 26312699-26313302; 6; HIST1H4E; INSIDE
GTCTGATCTGTGAAGGGTAGGGCCAGCAGGCAGCACCAAAGTTCCCGTATGCGCGTTTTCAGTCTTCATTTAGGTCC
GAATTCCCGGCATATAAGAATACTACCGTCGCTTGTTTTTCAGATTTTTGGGCTATTTTCGTTGGTGTGTTGGTCATG
TCTGGTCGCGGCAAAGGCGGAAAGGGACTGGGTAAAGGAGGCGCTAAGCGTCACCGTAAGGTCCTGCGAGATAAC
ATCCAGGGCATTACCAAGCCTGCCATCCGGCGCCTTGCTCGTCGCGGGGGTGTCAAGCGCATTTCTGGTTCATCTAC
GAGGAGACTCGCGGGGTTCTGAAGGTGTTTCTGGAAAACGTGATTCGTGATGCTGTGACTTACACGGAGCACGCCA
AACGCAAGACAGTGACAGCGATGGATGTGGTCTACGCGCTGAAGAGACAGGGACGCACTCTTTACGGCTTCGGCG
GCTAATGCTACCGTTAAACGACTCAGCATCTCGACTTCCCAAATCAAAGGCCCTTTTCAGGGCCGCCCACAGTTTTC
CGCAAAAGAGCTCATGACTTGTTAGACGATTGGTTGGTCTCTTTATAAGTTAATTGTTCCTGTCAA

TABLE 6
Primers and probes for MEIS1 MS-SnapShot.
BSP Primers (MEIS1)
Fwd.
5′ GGGTTTTTAGAGGTTAGGGGAA 3′
Rev.
5′ CAACTAAATAACCAAACCTCTCCTC 3′
MEIS1 A/B SNAPSHOT Probes

| | | Size | | | | |
|---|---|---|---|---|---|---|
| Probe | Sequence (5′→3′) | (bp) | Strand | UM | MT | μM* |
| MEIS1 A | T21 GGAGAGGGGGGTTATGATGTTAGG | 45 | sense | [symbol] | C | 0,2 |
| MEIS1 B | T14 GTTTGTTTGGTTTTAGAGAA | 34 | sense | [symbol] | C | 0,2 |

TABLE 7A
Differentially labeled CGIs between subgroups relative to blood

| CGI | Gene Name | Probe location | Subgroup |
|---|---|---|---|
| chr01-2072175- | PRKCZ | chr01: 002072146- | NMI-mt vs Ref |
| 2072389 | | 002072190 | |
| chr01-2106293- | PRKCZ | chr01: 002106497- | MI vs Ref |
| 2106715 | | 002106541 | |
| | PRKCZ | chr01: 002106664- | NMI-wt vs Ref |
| | | 002106715 | |
| chr01-47672249- | FOXD2 | chr01: 047671905- | NMI-wt vs Ref |
| 47672972 | | 047671953 | |
| | FOXD2 | chr01: 047672191- | NMI-mt vs Ref |
| | | 047672235 | |
| | FOXD2 | chr01: 047682633- | MI vs Ref |
| | | 047682689 | |
| chr1: 63554983- | FOXD3 | chr01: 063559476- | NMI-mt vs Ref |
| 63563059 | | 063559520 | |
| | FOXD3 | chr01: 063559476- | NMI-wt vs Ref |
| | | 063559520 | |
| chr01-68288825- | DIRAS3 | chr01: 068289060- | NMI-wt vs MI |
| 68289104 | | 068289104 | |
| | DIRAS3 | chr01: 068290072- | NMI-wt vs Ref |
| | | 068290116 | |
| chr01-71284766- | PTGER3 | chr01: 071284980- | NMI-wt vs Ref |
| 71286392 | | 071285024 | |
| chr1: 90955098- | BARHL2 | chr01: 090955137- | NMI-mt vs Ref |
| 90955445 | | 090955181 | |
| chr01-119333484- | TBX15 | chr01: 119333484- | NMI-wt vs MI |
| 119333719 | | 119333528 | |
| | TBX15 | chr01: 119333569- | NMI-wt vs Ref |
| | | 119333613 | |
| chr1: 163590111- | LMX1A | chr01: 163590167- | NMI-wt vs Ref |
| 163590435 | | 163590226 | |
| chr1: 173258989- | MRPS14 | chr01: 173259418- | NMI-mt vs MI |
| 173259419 | | 173259462 | |
| chr01-178464743- | LHX4 | chr01: 178470479- | NMI-wt vs Ref |
| 178471598 | | 178470532 | |
| | LHX4 | chr01: 178470597- | NMI-wt vs MI |
| | | 178470641 | |
| | LHX4 | chr01: 178470597- | NMI-wt vs |
| | | 178470641 | NMI-mt |
| chr1: 196153712- | LHX9 | chr01: 196154203- | NMI-wt vs Ref |
| 196154414 | | 196154247 | |
| chr1: 219117072- | HLX1 | chr01: 219117515- | NMI-mt vs Ref |
| 219117487 | | 219117568 | |
| | HLX1 | chr01: 219121301- | MI vs Ref |
| | | 219121352 | |
| | HLX1 | chr01: 219134113- | NMI-wt vs Ref |
| | | 219134157 | |
| chr1: 241719037- | SDCCAG8 | chr01: 241719439- | NMI-wt vs Ref |
| 241719368 | | 241719492 | |
| chr02-19424445- | OSR1 | chr02: 019415391- | NMI-mt vs Ref |
| 19425131 | | 019415435 | |
| | OSR1 | chr02: 019424965- | NMI-wt vs Ref |
| | | 019425009 | |
| chr02-45249374- | SIX3 | chr02: 045013420- | NMI-wt vs Ref |
| 45251726 | | 045013464 | |
| | SIX3 | chr02: 045015982- | MI vs Ref |
| | | 045016033 | |
| | SIX3 | chr02: 045015982- | NMI-mt vs Ref |
| | | 045016033 | |
| | SIX2 | chr02: 045085251- | NMI-wt vs Ref |
| | | 045085298 | |
| chr2: 54536404- | SPTBN1 | chr02: 054536311- | NMI-wt vs Ref |
| 54537878 | | 054536361 | |
| chr02-63134470- | OTX1 | chr02: 063134470- | MI vs Ref |
| 63134933 | | 063134515 | |
| | OTX1 | chr02: 063134470- | NMI-mt vs Ref |
| | | 063134515 | |
| | OTX1 | chr02: 063134803- | NMI-wt vs Ref |
| | | 063134847 | |
| chr02-66525936- | MEIS1 | chr02: 066525898- | MI vs Ref |
| 66527222 | | 066525942 | |
| | MEIS1 | chr02: 066526023- | NMI-mt vs Ref |
| | | 066526067 | |
| | MEIS1 | chr02: 066526791- | NMI-wt vs Ref |
| | | 066526835 | |
| chr02-70984709- | VAX2 | chr02: 070985519- | MI vs Ref |
| 70985764 | | 070985563 | |
| | VAX2 | chr02: 070985519- | NMI-wt vs Ref |
| | | 070985563 | |
| chr02-72224630- | CYP26B1 | chr02: 072227412- | NMI-wt vs Ref |
| 72228512 | | 072227461 | |
| | CYP26B1 | chr02: 072227537- | NMI-wt vs MI |
| | | 072227583 | |
| chr2: 73004709- | EMX1 | chr02: 073004783- | NMI-wt vs Ref |
| 73005568 | | 073004827 | |
| chr2: 85213468- | TCF7L1 | chr02: 085215511- | NMI-wt vs Ref |
| 85216104 | | 085215555 | |
| | TCF7L1 | chr02: 085216153- | NMI-mt vs Ref |
| | | 085216212 | |
| chr02-105864220- | NCK2 | chr02: 105864372- | NMI-wt vs Ref |
| 105865009 | | 105864417 | |
| | NCK2 | chr02: 105864443- | NMI-mt vs Ref |
| | | 105864487 | |
| | NCK2 | chr02: 105864906- | MI vs Ref |
| | | 105864950 | |
| chr02-111591678- | FLJ44006- | chr02: 111592037- | NMI-mt vs Ref |
| 111597436 | BCL2L11 | 111592081 | |
| | FLJ44006- | chr02: 111593125- | NMI-wt vs Ref |
| | BCL2L11 | 111593170 | |
| chr02-115635091- | DPP10 | chr02: 115636612- | NMI-wt vs Ref |
| 115637285 | | 115636656 | |
| chr2: 119329502- | EN1 | chr02: 119330694- | NMI-wt vs Ref |
| 119332035 | | 119330738 | |
| chr02-156892636- | NR4A2 | chr02: 156892639- | MI vs Ref |
| 156892878 | | 156892683 | |
| chr02-156893736- | NR4A2 | chr02: 156894489- | NMI-mt vs Ref |
| 156894685 | | 156894533 | |
| | NR4A2 | chr02: 156894576- | NMI-wt vs Ref |
| | | 156894620 | |
| chr02-171384729- | GAD1 | chr02: 171384729- | MI vs Ref |
| 171385226 | | 171384773 | |
| | GAD1 | chr02: 171385019- | NMI-mt vs Ref |
| | | 171385063 | |
| | GAD1 | chr02: 171385019- | NMI-wt vs Ref |
| | | 171385063 | |
| chr2: 172653235- | MAP1D | chr02: 172653288- | NMI-wt vs Ref |
| 172653630 | | 172653332 | |
| chr2: 174907710- | FLJ46347 | chr02: 174910857- | NMI-wt vs Ref |
| 174910885 | | 174910905 | |
| chr2: 176652334- | EVX2 | chr02: 176655532- | NMI-mt vs Ref |
| 176656692 | | 176655591 | |
| | EVX2 | chr02: 176656139- | NMI-wt vs Ref |
| | | 176656183 | |
| | EVX2 | chr02: 176656517- | MI vs Ref |
| | | 176656563 | |
| chr2: 176720618- | hsa-mir-10b | chr02: 176720535- | NMI-mt vs Ref |
| 176720921 | | 176720594 | |
| | hsa-mir-10b | chr02: 176720535- | NMI-wt vs Ref |
| | | 176720594 | |
| chr02-176737610- | HOXD3 | chr02: 176737609- | NMI-mt vs Ref |
| 176738187 | | 176737653 | |
| | HOXD3 | chr02: 176737609- | NMI-mt vs MI |
| | | 176737653 | |
| | HOXD3 | chr02: 176738050- | NMI-wt vs Ref |
| | | 176738094 | |
| | CENTG2 | chr02: 236438664- | NMI-wt vs Ref |
| | | 236438711 | |
| chr3: 11721578- | VGLL4 | chr03: 011721492- | NMI-wt vs Ref |
| 11721824 | | 011721547 | |
| chr3: 12983649- | IQSEC1 | chr03: 012983600- | NMI-wt vs Ref |
| 12984210 | | 012983644 | |
| chr3: 25680561- | TOP2B | chr03: 025681366- | NMI-wt vs Ref |
| 25681574 | | 025681410 | |
| chr03-129688131- | GATA2 | chr03: 129692644- | MI vs Ref |
| 129694023 | | 129692688 | |
| | GATA2 | chr03: 129698463- | NMI-wt vs Ref |
| | | 129698509 | |
| chr03-148591199- | ZIC4 | chr03: 148592447- | NMI-wt vs Ref |
| 148594390 | | 148592499 | |
| chr3: 148613030- | ZIC1 | chr03: 148613058- | NMI-mt vs Ref |
| 148613264 | | 148613102 | |
| | ZIC1 | chr03: 148619483- | NMI-wt vs Ref |
| | | 148619541 | |
| chr4: 4910534- | MSX1 | chr04: 004910620- | MI vs Ref |
| 4911092 | | 004910670 | |
| | MSX1 | chr04: 004910620- | NMI-mt vs Ref |
| | | 004910670 | |
| | MSX1 | chr04: 004910620- | NMI-wt vs Ref |
| | | 004910670 | |
| chr4: 30330303- | PCDH7 | chr04: 030333744- | NMI-mt vs Ref |
| 30333940 | | 030333788 | |
| | PCDH7 | chr04: 030333834- | NMI-wt vs Ref |
| | | 030333878 | |
| chr4: 41678680- | WDR21B | chr04: 041678597- | NMI-wt vs Ref |
| 41678940 | | 041678646 | |
| chr4: 85636684- | NKX6-1 | chr04: 085637474- | NMI-wt vs Ref |
| 85639823 | | 085637518 | |
| chr4: 94974810- | ATOH1 | chr04: 094974874- | NMI-wt vs Ref |
| 94975333 | | 094974918 | |
| chr04-111780013- | PITX2 | chr04: 111780048- | NMI-mt vs Ref |
| 111780343 | | 111780092 | |
| | PITX2 | chr04: 111780288- | NMI-wt vs Ref |
| | | 111780343 | |
| | PCDH10 | chr04: 134291474- | NMI-wt vs Ref |
| | | 134291518 | |
| | NR3C2 | chr04: 149586403- | NMI-wt vs Ref |
| | | 149586447 | |
| | TRIM2 | chr04: 154436244- | NMI-wt vs Ref |
| | | 154436288 | |
| chr04-155881084- | LRAT | chr04: 155881084- | NMI-wt vs Ref |
| 155881434 | | 155881128 | |
| | LRAT | chr04: 155881330- | NMI-wt vs MI |
| | | 155881378 | |
| | HAND2 | chr04: 174687934- | NMI-wt vs Ref |
| | | 174687985 | |
| chr4: 184255825- | FLJ30277 | chr04: 184255924- | NMI-wt vs Ref |
| 184258373 | | 184255968 | |
| chr5: 360287-360592 | PDCD6 | chr05: 000360593- | NMI-wt vs Ref |
| | | 000360637 | |
| chr5: 1161803-1162051 | SLC12A7 | chr05: 001162056- | NMI-wt vs Ref |
| | | 001162100 | |
| chr5: 3647468-3656054 | IRX1 | chr05: 003654466- | NMI-wt vs Ref |
| | | 003654525 | |
| chr5: 5192442-5193941 | ADAMTS16 | chr05: 005192514- | NMI-wt vs Ref |
| | | 005192558 | |
| chr5: 6790705-6791004 | POLS | chr05: 006790682- | NMI-wt vs Ref |
| | | 006790728 | |
| chr5: 54554812-54555385 | UNG2 | chr05: 054555175- | NMI-wt vs Ref |
| | | 054555219 | |
| | UNG2 | chr05: 054555391- | NMI-mt vs Ref |
| | | 054555442 | |
| chr5: 114542616- | TRIM36 | chr05: 114542773- | NMI-wt vs Ref |
| 114544119 | | 114542817 | |
| chr05-115810975- | SEMA6A | chr05: 115811349- | NMI-wt vs Ref |
| 115811393 | | 115811393 | |
| chr5: 127899858- | FBN2 | chr05: 127900012- | NMI-wt vs MI |
| 127900162 | | 127900056 | |
| | FBN2 | chr05: 127900012- | NMI-wt vs |
| | | 127900056 | NMI-mt |
| | FBN2 | chr05: 127900101- | NMI-wt vs Ref |
| | | 127900154 | |
| chr05-134390896- | PITX1 | chr05: 134390896- | MI vs Ref |
| 134393045 | | 134390940 | |
| | PITX1 | chr05: 134390896- | NMI-wt vs Ref |
| | | 134390940 | |
| | PITX1 | chr05: 134391093- | NMI-mt vs Ref |
| | | 134391137 | |
| | TRPC7 | chr05: 135720277- | NMI-wt vs Ref |
| | | 135720321 | |
| chr05-140724146- | PCDHGA5 | chr05: 140724562- | NMI-wt vs Ref |
| 140724826 | | 140724606 | |
| chr5: 140780945- | PCDHGA11 | chr05: 140781192- | NMI-wt vs Ref |
| 140781256 | | 140781236 | |
| | PCDHGA12 | chr05: 140791316- | NMI-mt vs Ref |
| | | 140791360 | |
| | PCDHGA12 | chr05: 140791441- | NMI-wt vs Ref |
| | | 140791494 | |
| chr5: 141235749- | PCDH1 | chr05: 141237048- | NMI-wt vs Ref |
| 141238152 | | 141237094 | |
| chr05-174091287- | MSX2 | chr05: 174091297- | NMI-wt vs Ref |
| 174092335 | | 174091341 | |
| | MSX2 | chr05: 174091410- | MI vs Ref |
| | | 174091454 | |
| | MSX2 | chr05: 174091473- | NMI-mt vs Ref |
| | | 174091517 | |
| chr6: 1326743- | FOXF2 | chr06: 001329813- | NMI-mt vs Ref |
| 1330210 | | 001329866 | |
| | FOXF2 | chr06: 001329813- | NMI-wt vs Ref |
| | | 001329866 | |
| chr06-1549606- | FOXC1 | chr06: 001551371- | NMI-mt vs Ref |
| 1560865 | | 001551415 | |
| | FOXC1 | chr06: 001551429- | NMI-wt vs Ref |
| | | 001551473 | |
| | FOXC1 | chr06: 001551500- | MI vs Ref |
| | | 001551559 | |
| chr6: 5947471-5950124 | NRN1 | chr06: 005947569- | NMI-wt vs Ref |
| | | 005947613 | |
| chr6: 10498025- | TFAP2A | chr06: 010498473- | MI vs Ref |
| 10498551 | | 010498517 | |
| | TFAP2A | chr06: 010498473- | NMI-wt vs Ref |
| | | 010498517 | |
| | TFAP2A | chr06: 010498811- | NMI-mt vs Ref |
| | | 010498870 | |
| chr6: 22677564- | HDGFL1 | chr06: 022677587- | MI vs Ref |
| 22678684 | | 022677632 | |
| | HDGFL1 | chr06: 022677587- | NMI-mt vs Ref |
| | | 022677632 | |
| | HDGFL1 | chr06: 022677587- | NMI-wt vs Ref |
| | | 022677632 | |
| chr6: 25834434- | HIST1H2AA | chr06: 025834650- | NMI-wt vs Ref |
| 25834701 | | 025834695 | |
| | HIST1H2BA | chr06: 025840937- | NMI-wt vs Ref |
| | | 025840981 | |
| chr6: 41176454-41177326 | NFYA | chr06: 041177112- | NMI-wt vs Ref |
| | | 041177165 | |
| chr6: 50895246-50896050 | TFAP2B | chr06: 050896068- | NMI-wt vs Ref |
| | | 050896112 | |
| chr6: 75968300-75968853 | COL12A1 | chr06: 075968669- | NMI-wt vs Ref |
| | | 075968713 | |
| chr06-101023927- | SIM1 | chr06: 101024299- | NMI-mt vs Ref |
| 101024343 | | 101024343 | |
| | SIM1 | chr06: 101024299- | NMI-wt vs Ref |
| | | 101024343 | |
| chr6: 101953488- | GRIK2 | chr06: 101953514- | NMI-mt vs Ref |
| 101953856 | | 101953560 | |
| | GRIK2 | chr06: 101953514- | NMI-wt vs Ref |
| | | 101953560 | |
| chr6: 108592365-108597232 | NR2E1 | chr06: 108595517- | NMI-wt vs Ref |
| | | 108595561 | |
| chr6: 126122028-126122524 | HEY2 | chr06: 126122132- | NMI-wt vs Ref |
| | | 126122176 | |
| chr6: 166499964-166503413 | T | chr06: 166502963- | NMI-wt vs Ref |
| | | 166503011 | |
| chr6: 169801163-169801511 | WDR27 | chr06: 169801059- | NMI-wt vs Ref |
| | | 169801108 | |
| chr7: 1029023-1029492 | hsa-mir-339 | chr07: 001029489- | NMI-wt vs Ref |
| | | 001029533 | |
| chr07-2082782-2083345 | MAD1L1 | chr07: 002083155- | MI vs Ref |
| | | 002083200 | |
| | MAD1L1 | chr07: 002083155- | NMI-wt vs Ref |
| | | 002083200 | |
| | MAD1L1 | chr07: 002121288- | NMI-mt vs Ref |
| | | 002121332 | |
| chr07-2724511-2724848 | AMZ1 | chr07: 002724511- | MI vs Ref |
| | | 002724555 | |
| | AMZ1 | chr07: 002724511- | NMI-wt vs Ref |
| | | 002724555 | |
| | AMZ1 | chr07: 002724629- | NMI-mt vs Ref |
| | | 002724673 | |
| chr07-8449587-8450236 | NXPH1 | chr07: 008449587- | NMI-mt vs Ref |
| | | 008449636 | |
| | NXPH1 | chr07: 008449587- | NMI-wt vs Ref |
| | | 008449636 | |
| | NXPH1 | chr07: 008450192- | MI vs Ref |
| | | 008450240 | |
| chr7: 19113449-19113833 | TWIST1 | chr07: 019113860- | NMI-wt vs Ref |
| | | 019113907 | |
| chr7: 27149139-27152087 | HOXA5 | chr07: 027150566- | MI vs Ref |
| | | 027150625 | |
| | HOXA5 | chr07: 027151311- | NMI-mt vs Ref |
| | | 027151355 | |
| chr07-27164708-27165039 | HOXA7 | chr07: 027164869- | MI vs Ref |
| | | 027164913 | |
| | HOXA7 | chr07: 027164869- | NMI-mt vs Ref |
| | | 027164913 | |
| | HOXA7 | chr07: 027164944- | NMI-wt vs Ref |
| | | 027164988 | |
| chr7: 27170441-27172987 | HOXA9 | chr07: 027170467- | NMI-mt vs Ref |
| | | 027170515 | |
| | HOXA9 | chr07: 027170467- | NMI-wt vs Ref |
| | | 027170515 | |
| | HOXA9 | chr07: 027170998- | MI vs Ref |
| | | 027171042 | |
| chr7: 27198331-27199622 | HOXA11 | chr07: 027198283- | NMI-wt vs Ref |
| | | 027198327 | |
| chr07-27219187-27220360 | EVX1 | chr07: 027245650- | NMI-wt vs Ref |
| | | 027245694 | |
| chr07-27251165-27252762 | EVX1 | chr07: 027251482- | MI vs Ref |
| | | 027251527 | |
| chr7: 35263447-35264743 | TBX20 | chr07: 035263668- | NMI-wt vs Ref |
| | | 035263712 | |
| chr7: 96469320-96469736 | DLX6 | chr07: 096469212- | NMI-mt vs Ref |
| | | 096469266 | |
| | DLX6 | chr07: 096469526- | MI vs Ref |
| | | 096469572 | |
| | DLX6 | chr07: 096469776- | NMI-wt vs Ref |
| | | 096469827 | |
| chr07-96488158-96489591 | DLX5 | chr07: 096489129- | NMI-mt vs Ref |
| | | 096489173 | |
| | DLX5 | chr07: 096489475- | MI vs Ref |
| | | 096489520 | |
| | DLX5 | chr07: 096489536- | NMI-wt vs Ref |
| | | 096489591 | |
| chr7: 119702299-119702721 | KCND2 | chr07: 119702676- | NMI-wt vs Ref |
| | | 119702723 | |
| chr07-121727243- | FEZF1 | chr07: 121727241- | NMI-wt vs MI |
| 121727884 | | 121727285 | |
| | FEZF1 | chr07: 121727336- | NMI-wt vs Ref |
| | | 121727380 | |
| chr07-155288454- | SHH | chr07: 155291537- | MI vs Ref |
| 155292175 | | 155291581 | |
| | SHH | chr07: 155291593- | NMI-mt vs Ref |
| | | 155291637 | |
| | SHH | chr07: 155291751- | NMI-wt vs Ref |
| | | 155291795 | |
| chr7: 158509937-158510390 | VIPR2 | chr07: 158509989- | NMI-wt vs Ref |
| | | 158510033 | |
| chr08-9798161-9799053 | hsa-mir-124a-1 | chr08: 009798455- | NMI-wt vs |
| | | 009798508 | NMI-mt |
| | hsa-mir-124a-1 | chr08: 009798593- | NMI-wt vs Ref |
| | | 009798644 | |
| | hsa-mir-124a-1 | chr08: 009800023- | NMI-wt vs MI |
| | | 009800077 | |
| chr8: 10624024-10624296 | SOX7 | chr08: 010624244- | NMI-mt vs Ref |
| | | 010624300 | |
| | SOX7 | chr08: 010624319- | NMI-wt vs Ref |
| | | 010624366 | |
| chr8: 11597006-11600365 | GATA4 | chr08: 011599544- | NMI-wt vs Ref |
| | | 011599588 | |
| chr8: 25955253-25955609 | EBF2 | chr08: 025955446- | NMI-mt vs Ref |
| | | 025955495 | |
| | EBF2 | chr08: 025955446- | NMI-wt vs Ref |
| | | 025955495 | |
| chr8: 55532724-55535078 | SOX17 | chr08: 055533948- | NMI-wt vs Ref |
| | | 055533992 | |
| chr08-65661156-65662687 | BHLHB5 | chr08: 065661235- | NMI-wt vs MI |
| | | 065661282 | |
| | BHLHB5 | chr08: 065662258- | NMI-wt vs Ref |
| | | 065662302 | |
| chr8: 72916429-72917309 | MSC | chr08: 072917197- | MI vs Ref |
| | | 072917241 | |
| | MSC | chr08: 072917336- | NMI-wt vs Ref |
| | | 072917380 | |
| chr8: 140700279-140700514 | KCNK9 | chr08: 140700520- | NMI-wt vs Ref |
| | | 140700564 | |
| chr08-142288380- | SLC45A4 | chr08: 142288398- | NMI-mt vs Ref |
| 142288718 | | 142288442 | |
| | SLC45A4 | chr08: 142288610- | MI vs Ref |
| | | 142288657 | |
| | SLC45A4 | chr08: 142288610- | NMI-wt vs Ref |
| | | 142288657 | |
| chr8: 145014900-145015187 | EPPK1 | chr08: 145015195- | NMI-wt vs Ref |
| | | 145015240 | |
| chr9: 959530-963276 | DMRT3 | chr09: 000959621- | NMI-wt vs Ref |
| | | 000959665 | |
| chr9: 21979774-21980108 | CDKN2A | chr09: 021979951- | NMI-wt vs Ref |
| | | 021979995 | |
| chr9: 23812413-23812667 | ELAVL2 | chr09: 023812373- | NMI-wt vs Ref |
| | | 023812424 | |
| chr9: 37024136-37028341 | PAX5 | chr09: 037024964- | NMI-wt vs Ref |
| | | 037025008 | |
| chr9: 101621613-101627382 | NR4A3 | chr09: 101627386- | NMI-wt vs Ref |
| | | 101627439 | |
| chr10-7489383-7495345 | SFMBT2 | chr10: 007491431- | NMI-wt vs Ref |
| | | 007491476 | |
| | SFMBT2 | chr10: 007491576- | NMI-wt vs |
| | | 007491626 | NMI-mt |
| chr10: 13556172-13556753 | C10orf30 | chr10: 013556159- | NMI-wt vs Ref |
| | | 013556211 | |
| chr10: 15294605-15294858 | C10orf38 | chr10: 015294592- | NMI-wt vs Ref |
| | | 015294641 | |
| chr10-21828640-21829645 | C10orf114 | chr10: 021828885- | NMI-wt vs Ref |
| | | 021828929 | |
| | C10orf114 | chr10: 021828954- | NMI-mt vs Ref |
| | | 021828998 | |
| | C10orf114 | chr10: 021829373- | MI vs Ref |
| | | 021829417 | |
| chr10: 76825135-76839606 | ZNF503 | chr10: 076834917- | NMI-wt vs Ref |
| | | 076834976 | |
| | ZNF503 | chr10: 076837381- | NMI-mt vs Ref |
| | | 076837425 | |
| chr10: 88112905-88117344 | GRID1 | chr10: 088115612- | NMI-wt vs Ref |
| | | 088115656 | |
| chr10: 94441310-94441717 | HHEX | chr10: 094441395- | NMI-mt vs Ref |
| | | 094441439 | |
| | HHEX | chr10: 094441744- | NMI-wt vs Ref |
| | | 094441795 | |
| chr10: 99068802-99071038 | FRAT1 | chr10: 099070245- | NMI-wt vs Ref |
| | | 099070298 | |
| | FRAT1 | chr10: 099070376- | NMI-mt vs Ref |
| | | 099070420 | |
| chr10: 101277153- | NKX2-3 | chr10: 101277900- | NMI-wt vs Ref |
| 101277910 | | 101277957 | |
| chr10: 102974173- | LBX1 | chr10: 102975940- | NMI-wt vs Ref |
| 102980053 | | 102975999 | |
| | LBX1 | chr10: 102976227- | NMI-wt vs MI |
| | | 102976273 | |
| | LBX1 | chr10: 102976227- | NMI-wt vs |
| | | 102976273 | NMI-mt |
| chr10: 106389558- | SORCS3 | chr10: 106392567- | NMI-wt vs Ref |
| 106392802 | | 106392611 | |
| chr10: 124891898- | HMX2 | chr10: 124892023- | NMI-wt vs Ref |
| 124892607 | | 124892067 | |
| chr10: 128863581- | FLJ45557 | chr10: 128864100- | NMI-wt vs Ref |
| 128864054 | | 128864145 | |
| chr11: 22319439-22319953 | SLC17A6 | chr11: 022319657- | NMI-wt vs Ref |
| | | 022319701 | |
| chr11: 31776637-31777992 | PAX6 | chr11: 031776609- | NMI-wt vs Ref |
| | | 031776653 | |
| chr11: 68274244-68274755 | MTL5 | chr11: 068274304- | NMI-mt vs Ref |
| | | 068274348 | |
| chr11: 117983446- | PHLDB1 | chr11: 117984800- | NMI-wt vs Ref |
| 117987106 | | 117984844 | |
| chr12: 1844001-1845219 | CACNA2D4 | chr12: 001844114- | NMI-wt vs Ref |
| | | 001844158 | |
| chr12: 40253533-40253742 | PDZRN4 | chr12: 040253480- | NMI-wt vs Ref |
| | | 040253529 | |
| chr12: 52726910-52727810 | HOXC4 | chr12: 052727781- | NMI-mt vs Ref |
| | | 052727833 | |
| | HOXC4 | chr12: 052727781- | NMI-wt vs Ref |
| | | 052727833 | |
| chr12: 79634857-79635257 | MYF5 | chr12: 079634813- | NMI-wt vs Ref |
| | | 079634857 | |
| chr12-94776044-94776377 | SNRPF | chr12: 094776044- | NMI-wt vs Ref |
| | | 094776094 | |
| chr12: 95164929-95165298 | ELK3 | chr12: 095165171- | NMI-wt vs Ref |
| | | 095165220 | |
| chr12-113657930- | TBX5 | chr12: 113325125- | NMI-wt vs Ref |
| 113659205 | | 113325174 | |
| chr12: 113609113- | TBX3 | chr12: 113609153- | MI vs Ref |
| 113609535 | | 113609207 | |
| | TBX3 | chr12: 113609259- | MI vs NMI-wt |
| | | 113609303 | |
| chr13-34950488-34951119 | MAB21L1 | chr13: 034950488- | MI vs Ref |
| | | 034950532 | |
| | MAB21L1 | chr13: 034950709- | NMI-wt vs Ref |
| | | 034950765 | |
| | MAB21L1 | chr13: 034950955- | NMI-mt vs Ref |
| | | 034950999 | |
| chr13: 35817738-35819004 | SPG20 | chr13: 035817882- | NMI-mt vs MI |
| | | 035817927 | |
| chr13: 57101588-57102323 | PCDH17 | chr13: 057101685- | NMI-wt vs Ref |
| | | 057101729 | |
| | PCDH17 | chr13: 057105585- | NMI-mt vs Ref |
| | | 057105629 | |
| chr13-94152191-94153185 | SOX21 | chr13: 094152286- | MI vs Ref |
| | | 094152330 | |
| | SOX21 | chr13: 094152463- | NMI-mt vs Ref |
| | | 094152507 | |
| | SOX21 | chr13: 094152463- | NMI-wt vs Ref |
| | | 094152507 | |
| chr13: 105943410- | EFNB2 | chr13: 105943362- | NMI-mt vs Ref |
| 105943788 | | 105943406 | |
| | EFNB2 | chr13: 105943488- | NMI-wt vs Ref |
| | | 105943532 | |
| chr13: 111763361- | SOX1 | chr13: 111764262- | NMI-wt vs Ref |
| 111764235 | | 111764315 | |
| chr13: 112249708- | TUBGCP3 | chr13: 112249984- | NMI-wt vs Ref |
| 112249928 | | 112250030 | |
| chr14: 19992581-19993863 | APEX1 | chr14: 019993551- | NMI-mt vs MI |
| | | 019993604 | |
| chr14: 23710894-23712060 | REC8L1 | chr14: 023711936- | NMI-mt vs Ref |
| | | 023711980 | |
| chr14: 28317076-28317375 | FOXG1B | chr14: 028317289- | NMI-wt vs Ref |
| | | 028317343 | |
| chr14: 33338698-33340189 | NPAS3 | chr14: 033339869- | NMI-wt vs Ref |
| | | 033339913 | |
| chr14: 36122289-36122589 | NKX2-8 | chr14: 036122225- | MI vs Ref |
| | | 036122274 | |
| | NKX2-8 | chr14: 036123184- | NMI-wt vs Ref |
| | | 036123232 | |
| chr14-36205192-36206099 | PAX9 | chr14: 036196442- | NMI-mt vs Ref |
| | | 036196486 | |
| | PAX9 | chr14: 036205602- | MI vs Ref |
| | | 036205647 | |
| | PAX9 | chr14: 036206112- | NMI-wt vs Ref |
| | | 036206171 | |
| chr14: 37137198-37138958 | FOXA1 | chr14: 037137114- | NMI-wt vs Ref |
| | | 037137159 | |
| | FOXA1 | chr14: 037137543- | NMI-mt vs Ref |
| | | 037137587 | |
| chr14: 56347822-56348040 | OTX2 | chr14: 056347712- | NMI-wt vs Ref |
| | | 056347756 | |
| chr14: 60045486-60047933 | SIX6 | chr14: 060046995- | NMI-wt vs Ref |
| | | 060047041 | |
| chr14: 60173732-60174416 | SIX1 | chr14: 060173710- | NMI-mt vs Ref |
| | | 060173754 | |
| | SIX1 | chr14: 060179528- | NMI-wt vs Ref |
| | | 060179574 | |
| chr14: 98781593-98783184 | BCL11B | chr14: 098782637- | NMI-mt vs Ref |
| | | 098782681 | |
| chr15: 50867751-50870991 | ONECUT1 | chr15: 050870481- | NMI-wt vs Ref |
| | | 050870525 | |
| chr15: 58083428-58085812 | FOXB1 | chr15: 058084349- | NMI-wt vs Ref |
| | | 058084393 | |
| chr15: 62984853-62985116 | ANKDD1A | chr15: 062984784- | NMI-wt vs Ref |
| | | 062984834 | |
| chr15: 65902540-65909629 | LBXCOR1 | chr15: 065908791- | NMI-wt vs Ref |
| | | 065908850 | |
| chr15: 72206924-72210097 | ISLR2 | chr15: 072206870- | NMI-wt vs Ref |
| | | 072206914 | |
| chr15: 74414579-74414893 | ISL2 | chr15: 074414745- | NMI-wt vs Ref |
| | | 074414789 | |
| chr15: 88158407-88159577 | ANPEP | chr15: 088158433- | NMI-wt vs Ref |
| | | 088158477 | |
| chr15-94688798-94689131 | NR2F2 | chr15: 094688839- | NMI-wt vs Ref |
| | | 094688885 | |
| | NR2F2 | chr15: 094688901- | MI vs Ref |
| | | 094688949 | |
| | NR2F2 | chr15: 094689087- | NMI-mt vs Ref |
| | | 094689131 | |
| chr16: 727072-727328 | NARFL | chr16: 000727383- | NMI-wt vs Ref |
| | | 000727427 | |
| | CBLN1 | chr16: 047869472- | NMI-wt vs Ref |
| | | 047869516 | |
| chr16-52873104-52882105 | IRX3 | chr16: 052879704- | NMI-mt vs Ref |
| | | 052879750 | |
| | IRX3 | chr16: 052879918- | NMI-wt vs Ref |
| | | 052879974 | |
| | IRX3 | chr16: 052880382- | MI vs Ref |
| | | 052880426 | |
| | IRX5 | chr16: 053528320- | NMI-wt vs Ref |
| | | 053528364 | |
| chr16: 66071900-66072846 | ATP6V0D1 | chr16: 066071973- | NMI-mt vs MI |
| | | 066072017 | |
| chr16-85098883-85102729 | FOXF1 | chr16: 085098933- | NMI-wt vs Ref |
| | | 085098977 | |
| | CNTNAP1 | chr17: 038089272- | NMI-mt vs Ref |
| | | 038089316 | |
| | CNTNAP1 | chr17: 038089272- | NMI-wt vs Ref |
| | | 038089316 | |
| chr17-44074280-44075233 | HOXB5 | chr17: 044026017- | MI vs Ref |
| | | 044026069 | |
| | HOXB5 | chr17: 044026017- | NMI-wt vs Ref |
| | | 044026069 | |
| | hsa-mir-196a-1 | chr17: 044074404- | NMI-wt vs Ref |
| | | 044074448 | |
| | hsa-mir-196a-1 | chr17: 044074457- | NMI-mt vs Ref |
| | | 044074501 | |
| chr17-58865328-58865618 | CYB561 | chr17: 058865281- | MI vs Ref |
| | | 058865326 | |
| | CYB561 | chr17: 058865281- | NMI-wt vs Ref |
| | | 058865326 | |
| | CYB561 | chr17: 058865512- | NMI-mt vs Ref |
| | | 058865556 | |
| chr17: 67627870-67631593 | SOX9 | chr17: 067630891- | NMI-wt vs Ref |
| | | 067630945 | |
| chr17: 71582000-71585125 | GALR2 | chr17: 071583191- | NMI-mt vs MI |
| | | 071583235 | |
| chr17-75531280-75531546 | TBC1D16 | chr17: 075531439- | NMI-wt vs Ref |
| | | 075531483 | |
| chr17: 75936057-75936582 | C17orf27 | chr17: 075936313- | MI vs Ref |
| | | 075936368 | |
| | C17orf27 | chr17: 075936313- | NMI-wt vs Ref |
| | | 075936368 | |
| chr17: 76404186-76404427 | KIAA1303 | chr17: 076404103- | MI vs Ref |
| | | 076404162 | |
| | KIAA1303 | chr17: 076404103- | NMI-wt vs Ref |
| | | 076404162 | |
| chr17: 77537903-77538626 | ASPSCR1 | chr17: 077538538- | NMI-wt vs Ref |
| | | 077538583 | |
| chr18: 894579-899574 | ADCYAP1 | chr18: 000899042- | NMI-wt vs Ref |
| | | 000899087 | |
| chr18-5186244-5187389 | ONECUT2 | chr18: 053259508- | NMI-mt vs Ref |
| | | 053259552 | |
| | ONECUT2 | chr18: 053259508- | NMI-wt vs Ref |
| | | 053259552 | |
| chr18-68359955-68362770 | CBLN2 | chr18: 068361299- | NMI-wt vs MI |
| | | 068361343 | |
| chr18: 74837994-74842232 | SALL3 | chr18: 074838538- | NMI-wt vs Ref |
| | | 074838582 | |
| chr18: 75565313-75565933 | CTDP1 | chr18: 075565319- | NMI-wt vs Ref |
| | | 075565363 | |
| chr19: 898548-898981 | ARID3A | chr19: 000898650- | NMI-wt vs Ref |
| | | 000898694 | |
| chr19-4059891-4060207 | MAP2K2 | chr19: 004059891- | NMI-wt vs Ref |
| | | 004059935 | |
| | MAP2K2 | chr19: 004060122- | MI vs Ref |
| | | 004060166 | |
| | MAP2K2 | chr19: 004060122- | NMI-mt vs Ref |
| | | 004060166 | |
| chr19: 9131907-9132918 | ZNF317 | chr19: 009132692- | NMI-wt vs Ref |
| | | 009132736 | |
| chr19: 12985960-12986259 | NFIX | chr19: 012986040- | NMI-wt vs Ref |
| | | 012986084 | |
| chr19-42586165-42586705 | ZNF569 | chr19: 042586544- | NMI-wt vs Ref |
| | | 042586592 | |
| chr19: 55553586-55553895 | NAPSA | chr19: 055553837- | NMI-wt vs Ref |
| | | 055553881 | |
| chr19: 57082654-57083180 | ZNF577 | chr19: 057082979- | NMI-wt vs Ref |
| | | 057083023 | |
| chr19: 60283718-60285792 | EPS8L1 | chr19: 060285492- | NMI-mt vs Ref |
| | | 060285536 | |
| chr19: 60907042-60907388 | EPN1 | chr19: 060907107- | NMI-mt vs Ref |
| | | 060907151 | |
| | EPN1 | chr19: 060907320- | NMI-wt vs Ref |
| | | 060907364 | |
| chr19: 62043096-62043807 | PEG3 | chr19: 062043010- | NMI-wt vs Ref |
| | | 062043054 | |
| chr19: 63321122-63322053 | ZNF329 | chr19: 063321824- | NMI-wt vs Ref |
| | | 063321868 | |
| chr19-63420090-63420541 | ZNF274 | chr19: 063420437- | NMI-wt vs Ref |
| | | 063420481 | |
| chr19-63784504-63785085 | MGC2752 | chr19: 063784639- | NMI-wt vs Ref |
| | | 063784683 | |
| | MGC2752 | chr19: 063784804- | MI vs Ref |
| | | 063784848 | |
| | MGC2752 | chr19: 063784804- | NMI-mt vs Ref |
| | | 063784848 | |
| chr20: 9444472-9444893 | C20orf103 | chr20: 009444951- | NMI-wt vs Ref |
| | | 009444998 | |
| chr20: 21433933-21444714 | NKX2-2 | chr20: 021439947- | NMI-wt vs Ref |
| | | 021439991 | |
| chr20-22505518-22507240 | FOXA2 | chr20: 022505822- | NMI-wt vs MI |
| | | 022505866 | |
| chr20-22510737-22514165 | FOXA2 | chr20: 022505888- | NMI-wt vs Ref |
| | | 022505938 | |
| chr20-22514762-22515148 | FOXA2 | chr20: 022507864- | NMI-wt vs |
| | | 022507908 | NMI-mt |
| chr20: 23294035-23294650 | GZF1 | chr20: 023294489- | NMI-wt vs Ref |
| | | 023294536 | |
| chr20: 25011839-25013525 | VSX1 | chr20: 025011908- | NMI-wt vs Ref |
| | | 025011952 | |
| chr20-35582018-35583550 | NNAT | chr20: 035582918- | NMI-mt vs Ref |
| | | 035582969 | |
| | NNAT | chr20: 035582918- | NMI-wt vs Ref |
| | | 035582969 | |
| chr20: 36785545-36790786 | SLC32A1 | chr20: 036787891- | NMI-wt vs Ref |
| | | 036787941 | |
| chr20: 38749965-38753401 | MAFB | chr20: 038753128- | NMI-mt vs Ref |
| | | 038753174 | |
| | MAFB | chr20: 038753269- | NMI-wt vs Ref |
| | | 038753318 | |
| chr20: 49542203-49542863 | NFATC2 | chr20: 049542266- | NMI-wt vs Ref |
| | | 049542311 | |
| chr20-54012011-54014085 | CBLN4 | chr20: 054013134- | NMI-wt vs Ref |
| | | 054013178 | |
| chr20: 54411932-54412178 | CSTF1 | chr20: 054412143- | NMI-wt vs Ref |
| | | 054412187 | |
| chr20: 55680656-55681057 | TMEPAI | chr20: 055680906- | NMI-wt vs Ref |
| | | 055680952 | |
| chr20: 56860125-56860442 | GNAS | chr20: 056860480- | NMI-wt vs Ref |
| | | 056860524 | |
| chr20-60061693-60062033 | TAF4 | chr20: 060015181- | MI vs Ref |
| | | 060015225 | |
| | TAF4 | chr20: 060062094- | NMI-wt vs Ref |
| | | 060062139 | |
| chr20: 62371744-62371958 | PCMTD2 | chr20: 062371900- | NMI-wt vs Ref |
| | | 062371944 | |
| chr21-33316999-33322115 | OLIG2 | chr21: 033318624- | NMI-wt vs Ref |
| | | 033318681 | |
| chr21-36990008-36995832 | SIM2 | chr21: 036991619- | NMI-wt vs Ref |
| | | 036991663 | |
| | SIM2 | chr21: 036999107- | MI vs Ref |
| | | 036999151 | |
| chr21-43350621-43351134 | HSF2BP | chr21: 043902052- | MI vs Ref |
| | | 043902096 | |
| | HSF2BP | chr21: 043902052- | NMI-mt vs Ref |
| | | 043902096 | |
| chr21: 45511336-45511549 | POFUT2 | chr21: 045511551- | NMI-wt vs Ref |
| | | 045511595 | |
| chr21: 46892946-46893236 | PRMT2 | chr21: 046893181- | NMI-wt vs Ref |
| | | 046893225 | |
| chr22: 18453364-18453931 | DGCR8 | chr22: 018453720- | NMI-wt vs Ref |
| | | 018453764 | |

TABLE 7B
Sequences of differentially labeled CGIs between bladder cancer
subgroups relative to blood

CGI: 561 range = chr1: 63554983-63563059
CGGGCAGGCCCAAGCTGCGATGTGGAGAATTCGATGTCCGAGCGACCTCCTCGGAGGAGTGGGT
CGAGTTAAATATAACCGCGCGAATGGAATGGCGCTAAAAATAAGGCAGCAGCTGGCCTGTCCAC
AGCCCTGTCCCGGGAGGGGCGGGGGCCCCAGTGGTCTTGGGCAGGAAGGCCGCGTCCGGCCCAG
GGGCGAGAAGGCTGCGGCGTCCGCAGCCAGGGCTGGAAGGCCTGGGAGGCCGCGCTCTGTGGGC
CCCGGGGCCTCCATTCGGGCTGGGTCGCGGGCCTGGACGGGGACTGTCCAGAGGCATCCGAAAG
CCAGGCCAACTTGCCTGGACGTAACAAGACGGAAGGGCTGGGCGCTGAGGTCCTGCCAGCCCGG
CCGCCAGAGGGAGCTGAGCGCCAGAGGAGGACAAGCCGAACCCTTCAGGAGGCCGGGCGTCTCC
GGAGACCGAAGCGCCGGAGGACCCGAGGAGGTCTGCCCCGCGCGCTGCTCTGGAGACTCCCGGG
GCGGGTGGCGCTCGGCCTTTCCGCTCCCTTCCTTCCCACAAGTCCCTTCCCGCGCGCGCCCCACGG
CCCTGCCCGCCCTCCCGCGTCAGCGCCCCAACCGTCAAGCCAGCAATTGAAACGTTTCCAAAACG
GTCTATTTATTTGCTCCCAATAAATCGATCGGCGGTGATTAAAGAATCGATGTGGCCTGGGTGGG
CGAGTCGCTTGAGGGGAGGGATTGGGGGCTTTCGCCCGGCGCCTGCAGGGAGGCCGAGGGCGGG
CGCGGGCCTGAGGGAGGCGTGTCCCGCCCGGGCCACACCCGAGGACCCGACACCTGGGCTGGCA
GGCCCCGGCAGGCAGCGTTCCCTCCGGCGGAGAGGGGCGCGCGCCCGCCGCCTGCTTTCCTCGGC
CCCTCTCGCCTTTCTCGCGCGCCGGGGAGGCTGTGGCCGCCAGTGGCTGCGGAGCTGCTCAGAGG
CTTTTGTTGCTCCTCGGCCGGCTGAATGGGGATTTTGTAAAGCGGGACAGATAAAAATGAGCAGC
ATCATATTGTTTGACAGAATGATCTCGCATGATGAAGTGTCGGCTCCGAAGGGGGTGAAAATGGT
GAATTCCTAAAAACCCAGCCCTGGGCTCCTCCTCGAGCTGCCGGTAGCCTGGAGGGACCCAGCGG
ACAGCCGGGCCTGGCCGCATCGCTCCAAACGGTGTCAGAAAGACTCCGGCTTTCAATGCCAAGTC
ATTTTTAAGCCCCGATCCTGTCCAGGACCTTTCTCCTCGTGGATGAAAAGAACAATTTTCGAGAG
AAAGGCTCGTTTTTATTAAATCCGACATGCTGCTGATAACTCCATGCTAATGTGAAATAATTAAC
ATAATAGCCATAATTAAAAGCACGCTAACAATGCCATAAATTTATCACACAATTTTACTAGCTTT
CTGCCCCTAACTGCTCTCTCATCGTTAATTAAACGTGTTGCCTTTTACAGAATGGATGTTTATATA
TTTCCAATATAAATAAATTCGAAACCATCCTCTCTCTCTTCCTCTTTCTCTCCTCCTTTCCTTTTGGT
CTCTCGCCATTTACAGGCACGCCTTGGCGTGGACCCTGAGTGGCAGACATCTTGAAAATAAATGA
AGTTTTGAGATGCAAATCCAAACAAGAACATTAAAATAGCCTCTTTTTTTCCACCCCGAAAAGAT
CCGGAGAGGTATACAAGGGGGTAGTGGTGGGTAAGAGAGTTGAAAATCCCCCGCTTTGGGAAAT
GGAAGTAATCTGGGTGGGTTGGGGCCTTGGGTACCACCTCTGCCCTTTCCCACCTTCCTTGGTGGC
GGCCATCCAGACAAAGAGGCCGGTAATAGTTTAACAAATCTATGAAGATTTTCAAGAAGCAGCA
GACTTTGATTGTTGCGGGCGCGGGGGTGTTGGGGAGAAAGGAGGGGAATTTTTCTAATAGTCCCA
CCCACGTTTTGCTCCCTCTTGGACAAAGAGTAACTACTCTTGGTGGGGGACGCGCCCTTCACTCCG
CGGAACCTGGTCCCAACTCCCCGTATTGTAAGAAAAGTGCACCCGCGCGCGGGCATGATGATTCT
ATCTCACATCGCGCCAACGACTTATTCAAGCCACTGGCACTGTCTCTGACTTAAAAGAGGAGAAA
AGAGGCATATGGGTTCACTTGGGCCTGGTGAGGGGTAGGTGGGCAATTCCCGCCTTCCGCACTCT
AACCGTGCCCCTCCTCCAGTGTTGACCACCTAAGAACCCAAAATGAGCTGTAATTAATTTCCCTTT
CTCCATCATAAATTTTTCTATCCATTTCTTCCCCCCCATCCCCCCACTGGACGCACACACTAAATCT
CCCCTCCCCTGGAGACGTCTCAATTTCCTTCCTATCGATCCGGACTCCATTCTTCTTGCCTCCTGTT
GCTAGAACCTAGATCCCCACTCCCCGCACCCCTCATTCCCACCGCGTCCAGGTGGCTTTCCCAGCG
GGGTACCATGTACTCTGCCCGCTCCAGAGGAACCGAAGGGGTTTCATTCCATTCTCCTTTGGTTGA
AACATTTCAAACATTTGAGCAGGTGAGGCAGCTGGCTGCCATCTTCCTTTTTAAATCTCTCCTGGG
AAGTTCGCTTGTTGAGACTCAAAGAGTCACTCAAACTCATAATTGCGTGTGTGTGTCTACTCATTC
TCCCTCTATCTCTCCAATAACCCTTTGAGACTCAGAAACTTTTTATCCACATACACCCTTTATCAC
ATTTTCTTCCCCCCACTACATGTGTCTCACTTTCTCTCTGTATCTGTCTCGCTTCTTCCGTCTCTGTC
CTACAGCTTGGCGGTAACTGACGACCTGTGAGCTTTTAGCTGCAAACTGCAACTACGCGGCAAAC
AATTTATTTAGCCCGACATCTAGCCGGTCTCCGGCAGGACCCTGCACCGCGTCGGGATCGGACCC
TTCCGCTGGGGCGGCCTCCTGCGTCAAGGCCAGCAGGAACCTTCCTGTCGCCCTCCCCGGCCGCC
GCTTCGCCTCCTTCCCGCCCCCGGAGGTTGTGCAGGCGCTATGGTCCGCCTGGAGGGAGAAAGCC
GGCGGCCGGTTCCTGAGCCGAGAGCGGCCGCGGAAAAATCCTCTGCCTCCGCTGGAAATCGATAT
TAGGCCGGCGCGGGCGCGGGACGTCGGGGCCGCAGCCAGTAGGTTGTGCACGTCTCATCATTTAG
CTAATCGAGTCGAAAAGTTTCTGTAAGGGCCGGACCCAGCATCAGATGGTAACACTGATTGAACA
AGAGATTAGCACAATAGATCTCTAACCGAGGGGAAGCGTTGCTTTTCACGCTACGCGCCGTAATT
AATGGTATGAATCAATTAATTTGACTTTTATTGTGTCGAAGGAAAAAAGCGCAACAAATGGAACC
GGCAGCTGGGAGTTGTTCGTCCTCCACCCCCTTCCCCAGGGAGGTTCCAAGGAGACACCGGGGAA
TGGACGGATCAGGCTGGGCCGTGGCAGAGGGAGGGTAGGAGGCAGCGACCAGCAGCGTGGAGG
GAGTCCAGAGAGCTAGCCTCTGCGGACGGCGGAATCGAAATTAGGCTCATTTGGAGACTACTTCG
AGACCGGTGAGGGGAGCCCTGTAGCCACCATCCTCCGGCGCGCATCCACACATACTAGTCCACGC
GGGCCCAGCCACCAAGGCCGCGGCAGGGCCAGCGCTGCGCCCCGGGCCCCTGCCTTTAGGGCTG
GGCAACCCAAGCAGAGCAAAGGAGGTTCCTGAATGTGTAAATTTCCGCTTTTTAGCTTTTTTTTTT
TTTTTTTTTGGACCTTCCGACACTTCGGTTGCTGAGGCAGTTGCAGACGCGACCTCTGCAGTCCTG
GGCGATGGCCAGCCAGCTCAGCTCGGGTCGGTTTCGCGGAAAGCTGTCTAGACGGCATTGTAAAC
GGTTCGGAGCCTGCGGGCCACAAAGCTGTGGAGCTACGGAAATCAACTCTGAGATGCGTTTTAGG
GCCGTGTGCAACCTCGGGATCATTTAGATAAAGAAAAACTGTGGAGGTTGGCGGGCGTCTCAGG
ATAGTGTCACCACCCCCTACCCTGCTCCCAGCCTCAGATGAGTAGTGTTATATCCTGGGAAACTGT
CTAATGGGGATGAAAGTCAATCTGTGTGTCTCAATGCCTGTAATGAAGCAAGTTTACAGATTTTT
AAATTTTTATTTTTATTTTATTGAATTATTTTTGGTGTGTCTAGGCCAAGGAAAGAGGAGATCGTG
GGTGGGGAAACAGACTGAGGGAATCAGAAGCACCACTGTCCATCCGGAATTAAATCCACATCCC
AGCATCTTCTGCAAATATTTCACTAATTATTTCCTCTCGGAACTCCTCCCCTCGTGCTCCTTCCTCT
GGTGAGGCCGGCGCTCCCCTCCCAGGCCGCAGCGGACAGACAGGGATTGGGTTCCGTGTGCCTGC
CACACCAGGCAGGCTCTTGCGGCTCCCAACTAGGCGGCCTAAATGAGGGAGGAAAGAGGAGGCG
CATCGCTGATTCACCGCGTCAAGAGCACTGACTTTCCTTGGAGGTGTGAGGTCCACGCACCCCAG
CCACGCACTTGGGGGTCGGTTTGCGGTGCCTCCCCCTCCAGTCCCAGTGAAATCCCCACAGTTTTT
CCTACTATCACTGACTTGCCTTGCACTCCGCGTGCATTGGCCACACATCCTCGCCTCCTCCACCCG
CTCCGCCGCCGGTTTTCTTGGAAGTTAAATCTTGGAGGATTTGTCCACACCTTAAGAGAAGAAAA
TCCACGTTAGCTGGCAGCAACGGAGATCCCAGCATGCTGGCATGCCCAAGTCTGCCCAGGTTCCC
CCAAGGCCATGCCCGCCGCCCGGGAAGTCACTGCCCGCACCCCTCACGTTTCTTCAGCCGCCCCT
GGGCGCTGCGTCTAACCTGAAGACACCAGGCCTCTTCCCGGATCCACTCGACTTACCCAGGCCGC
TGCCAATCCCAGCTCCTTCCCCAGCGCCTCATTTCCGATTTTTTCATATGCTAAGTCGTTTAACAA
CTCCAAGTAGCCAGTTATGGCTTCTTTATTTATAGGTTCCCTGCTATTTTACGTCGTTTTTATTTCT
CTCGGCAACTATTCTAGTAGATTAATCAATAGCCATTTTCTGACCTTCGGGAACCCCAGCTGATGC
TTTTTGTGGCCGCACGAAAAAATACATACAGGAAAACACGCCCGCATCAAGCCGGGAAAGAGCA
GGTAGGACCTGAGTGGTTTGGTTGGGGGAGGGGGAAAAAGACATCTCAGCAGGTGTCTTCCCCG
GAATGAGCACTGAGGCCAGAGGGGAATCTGAAATCTAATTAGCAGGAGGGAGCCGGGTGCGCTG
CTCTTACTCTTTAAAGCTAAAAACAATGAAACAAAAAGCAAAACAGAGACTAAGTTTTGCTTTTT
AAAACACGATATGGGAACCTCGTTCTAGGTCGCCCAGTCCCTGTCTAAGGAGTGTGACAAAGTGG
GGGGGAGAAGGGCGGAAGGGAGAGGGGGCGGGGAAGGCAGGGCAGCGACAGTCGCACAGTCCC
GCGGACGCTCCCAGGCCCACGCCCTGACTCGCTCACACCCACCCACACTCACACCCACCCGCTCC
CTGGGCCCCAGGGCCCGGATCCAGCCTGGGTGGGGGGGTCTCCGGGCGGGCCGCAGCGCCCTCC
GTGCCCCGGGGATGCTGGCGCACAGTGCGGAGCGGAGTTGCGCGTCTCTCGTCCCTTTGTTGACA
ATTCCCTGAACCAACTTGAGTTTGGCCGGCTCGGCCGCGGCCCTGACGTCACGCACGGTCACGTG
GCCCCGCCTCCCGCTGGATCTTTAAGTAGAAAGTAATCTATCAGGCCAGTCCTTAAAACGGGACT
TTCGACTACCGGGGCTTCGGCGTCCCTGACACCCAGCCCCCTGCCCCCCCGCTACTGTCCCTGCCC
GCGCCCTCCCGAGCTGCTCGGCGCCCGGCGTCCCGCGCCCGCCTGGACCGCTCCTGCGCCCCACG
CCAGGGCCAGAGGCCGAGGAAGGCGGGCTAAGTGAGGGGGCGCGGCGTGGAGAACCGCCGGGG
CCGGGAGCGGTAGCGAGCGCCTAGTACCGAGCGCCAGGGACGGCAGGAGTTCGCGGAGCGCGGC
CGCTGGGGGCGGACGGCAGAGCCCGCGCCACGCGATGCGGGGCCGCCGAGTGTGAGCTGAGCCC
AGCGGGCCCCAAGCCACCTGCGGCCCCCTCCCCTCTCCCTGCCCCCCATCTTTCGGGGGCACTCA
AACCCTCTTCCCCTGAGCTCCGTGGCAGCCCCCGAACACCCTCATCGCCCGCTGCCCCCTCCCCGC
CGCCGCTACCAACCCCGAGGAGGGATGACCCTCTCCGGCGGCGGCAGCGCCAGCGACATGTCCG
GCCAGACGGTGCTGACGGCCGAGGACGTGGACATCGATGTGGTGGGCGAGGGCGACGACGGGCT
GGAAGAGAAGGACAGCGACGCAGGTTGCGATAGCCCCGCGGGGCCGCCGGAGCTGCGCCTGGAC
GAGGCGGACGAGGTGCCCCCGGCGGCACCCCATCACGGACAGCCTCAGCCGCCCCACCAGCAGC
CCCTGACATTGCCCAAGGAGGCGGCCGGAGCCGGGGCCGGACCGGGGGGCGACGTGGGCGCGCC
GGAGGCGGACGGCTGCAAGGGCGGTGTTGGCGGCGAGGAGGGCGGCGCGAGCGGCGGCGGGCC
TGGCGCGGGCAGCGGTTCGGCGGGAGGCCTGGCCCCGAGCAAGCCCAAGAACAGCCTAGTGAAG
CCGCCTTACTCGTACATCGCGCTCATCACCATGGCCATCCTGCAGAGCCCGCAGAAGAAGCTGAC
CCTGAGCGGCATCTGCGAGTTCATCAGCAACCGCTTCCCCTACTACAGGGAGAAGTTCCCCGCCT
GGCAGAACAGCATCCGCCACAACCTCTCACTCAACGACTGCTTCGTCAAGATCCCCCGCGAGCCG
GGCAACCCGGGCAAGGGCAACTACTGGACCCTGGACCCGCAGTCCGAGGACATGTTCGACAACG
GCAGCTTCCTGCGGCGCCGGAAACGCTTCAAGCGCCACCAGCAGGAGCACCTGCGCGAGCAGAC
GGCGCTCATGATGCAGAGCTTCGGCGCTTACAGCCTGGCGGCGGCGGCCGGCGCCGCGGGACCC
TACGGCCGCCCCTACGGCCTGCACCCTGCGGCGGCGGCCGGTGCCTATTCGCACCCGGCAGCGGC
GGCGGCCGCGGCTGCTGCGGCGGCGCTCCAGTACCCGTACGCGCTGCCGCCGGTGGCACCGGTGC
TGCCTCCCGCTGTGCCGCTGCTGCCCTCGGGCGAGCTGGGCCGCAAAGCGGCCGCCTTCGGCTCA
CAGCTCGGCCCGGGCCTGCAGCTGCAGCTCAATAGCCTGGGCGCCGCCGCGGCCGCTGCGGGCA
CAGCGGGCGCCGCGGGCACCACCGCGTCGCTCATCAAGTCCGAGCCAAGCGCGCGGCCGTCGTT
CAGCATCGAGAACATCATAGGTGGGGGCCCCGCGGCTCCTGGGGGCTCGGCGGTGGGCGCTGGG
GTCGCCGGCGGCACTGGGGGTTCAGGGGGCGGCAGCACGGCGCAGTCGTTTCTGCGGCCACCCG
GGACCGTGCAGTCGGCAGCGCTCATGGCCACCCACCAACCGCTGTCGCTGAGCCGGACGACTGCC
ACCATCGCGCCCATTCTTAGCGTGCCACTCTCCGGACAGTTTCTGCAGCCCGCAGCCTCGGCCGCC
GCCGCTGCTGCGGCCGCCGCTCAAGCCAAATGGCCGGCGCAATAGGGACGCGCCAATGGCCGGG
ACCCAGGGTCCGGCGGCGGCCTCGAGCAACAAATGCACCTCCAGGCTGCGCGCCCTGTCCCAAG
CCCGGTCCCGGTCCCGCTGCCCAATCCTGGACTCTGCCTCTCCCCAATTTCCTTTCCCCTGAGCCC
CCAACGCCTACCTTCCGCGGCCTCCATCCCCTCGCGCACACCTAAGCTGGTCGAGCAAACTCACC
GCGCGCCCGCCGGGGATAGCTTTCCATACAGGTAAAACCGAAAACCGAATTTTCCAAAAATGCA
CCCCGACGGCGCCTGCTCTTAGTACCG
CGI: 26 range-chr1: 90955098-90955445
CGCGTCTGCTACCAGATGCGGCTCCGGGGGCTCCATGGTGACTGAGATAGGAGAAGAAGGCGCC
GTCCCTACGGTATCAATCTCCGAACAGGGAGATGGGGTGGCCTGACTCCTAAAATCCGCGGTCCT
GGCCTCACCGAGCGGGCGGAAATCTCCATTCATCATGCCTGGGCTGCCTGAACTGGCACTGGACA
AAATCGTGTCTATTCCAAAACTCGACCCGCTGGCCCCTTCCATTGTCATTGCTACATGAGGTCCAC
GCCACTCCGCCGTTCAGCAGCCGCCCCGAACCAGCGAAGAAAGCTATCGATCGTAAAACAAAAT
AAACACCAAACAATGTTGCCGCCG
CGI: 29 range = chr1: 163590111-163590435
CGAGAGGCCCGGCCACCGGCTCGGGAGCTGGGCCGGTCGCTTTGGGAGCGGATGAGGAAGGTTA
GGAGAAGCAGCGAGATAGATCCCAATTTTACAATTCTATTTTCTTTCGGTAGGGTCTCGGCGTCCT
GGGCCACGTTGAGAGCGAACGTGGGCCGAGCGGAGGACACAGAGTAAAAAGCGACGCCCGCTG
TATACATAAATCCGCACCCGCTGCCCGCCCGGGTACTGCCTGCTCTGGCTTCCGCTCTCTTCCGAG
GCTGGGCAAGTCCAAAAGTTCCCGAAAGGGGGGTTCAAAGAGGGGCGCTCAGTGCACGTGATTT
CG
CGI: 32 range = chr1: 173258989-173259419
CGCGGAAGAGGGCGGAAGGAGAACCCCTGGACTGGTCCTCACCGAGCACTACAGGTGGCAGAGC
CGTGGGGGACCCGTGGGTTATGAGATTCAGCGGTGAGGGGTAGCGGTGTGGATAAAAGTAGAGG
CCTGACCTGCTTGAACGTCCGCAGCAGCGAGCCCAGCATGAAGGCCGCCATGTTGTCCGCTACAA
ACTACAAACCGCTGAAACTTTATTGACACTGCCGAATAGCGCAGGACACGAGGGGGGGGCCTG
GGCCGCCTGGGGCGGAGCTGGGAGGTGGGTGGGAATGTGGCCTACAGGAAAGCGGGGTTAGTTT
TGGGATCGGAGCTCTCGCGATATTTCACGGAACTTGGGCAAATGACAAATCTGAAGGCGCCGCGT
TGATGCTGATGGCGCAGCCAGACCCGCCCCGTGTGTTGCAGCCCG
CGI: 58 range = chr1: 196153712-196154414
CGTCTGGCCAAAGGCGCCCAGCTCAACGGCCGCGACGCGGTAAGGAAGGGCTCCGCCGCGGCGG
TAGCCGGGCACCCCTGCGCCCTCTGTTAAACCAAGCCGACTCCCTGGCCGGTGGAGAACCGGAGC
GGCGGGTCGTAGAGACGTCCGCTTGAGGGTGGCGGGTGAGTTTTCTCCCGTCCACCTATGCCTAC
AGGGCTAAGAAACGGTCGTTTTTCTGAATCGAAATGTCTGCAGCTGGCTATCCTCCGTCGCAGCT
GATTCCGAAAGAGCAGGGAGAGGAACTGGGAGGAGGTGGGATGGGGGGTGGTGGCATTCTCTTC
TACAGACCTCAAGGTTCCCTTGATTCCGGGGCAGGCGTCTCCCCGGCGAGGATCCGCAGCTCCGA
GGGCAAGCTGGGCATAAGCAATAGGAGGACGGCGCGCTGCCGAGGCGTCCGAGCCAAGCAGGA
GCCCAGGTGGCCTTAGTCTCTGGGCCTGATGACCGGACCTGTGGAGTAGATTCCGACGCGACTGG
GTCATTTCCAGTTCTCTAGACGCTCGGGGCTTGGGACCCCTAACCGAGAGAATCTCAGGGTTTCT
GGCATCCCGACTCAGTCCCTCTAAGGAGACAGCACTACGTTTAGCGCCAGGACCCGGCGGGTGTC
ATGTGTAGGGGGAAATCAAATAAAACATACGGAGGAGCGCGGCACCCAGTCGAAACG
CGI: 43 range = chr1: 219117072-219117487
CGGCGGGGGTCGCTCCAAAGACTTGTATTTCGCGTTTGCCTCCGGGAGCTGGGAGTAAGGCCTTG
GATGGCGCCGACGCGGTTGCGAGGAAGCTGAGGCCTGGGAGAGCAAGGGGCGCGCAGGCGAAG
TTGCAACTTGCACTCCAGCCGCGGGCCTGGCGGAGAAAGGGAGGCTCGCGGCGCCGCGAGGAGT
CGGCGGGCCTCGGGGCCTCGCTTTCGCCGCATCTGCCCAGCGCTCCGGGCCTTGAATCTCGGCAG
ATGCGAGTTGTGGGCACCTAGGGAACCCTGAGGACTCGCATTCCCCCGGGTCTGTATCCCGTGCC
CACCCCGGAGCGTCGCAAACCTTGGAAAGGGTGAAAGCTGATAGGGGAGTCTTATTTCTTAAGA
GAGAGGGTGAGGGCGCGCGGCGCTGCCTCG
CGI: 25 range = chr1: 241719037-241719368
CGGAAGAGGTGGACCGGCTGCGGACCCAGGTACTGTGCAGAACGCGGCGCAGGTGGGAGTCCTT
GGGCGGGCGTCTACAGGTGGATCTTTTATGCACCTCAACAGGCCGGCTTTCTCACGGATCACATC
GGTTAGCAGTAAGCTGGGAGGGAGGTCCCGAAGACTGTGCTGGGCACAGTGCAGGACCCAGAGA
AGCGGAGCCATGGGCCCGTCCTGGTGAGGGAGTTAAAGTCCGTGCAGGTGAACAGACCCCGGCC
CGCGAATCAACGAACCACACCGAGTGCCAAGTTGCGCCGTGCAGGCTGCGGAGGGTGCGAGAGC
AGGGAGAAGCG
CGI: 132 range = chr2: 54536404-54537878
CGAACGCAACATCAAGGGCGAAAAGAGGCTGCACAAATCACGTAGAAAATTAGACCGCCTCTTT
CCATGGCGATAATCGAATTAGCCTCAAATTAAACGTGAAATTGGCCCTCTCCGGCTGTGGAGACA
AAGCCCCTATTGTCCTGCTCGGGTAGCGGCAGCAGCAGTGCCCACCCCGCCCCGGCCAGCCTGGT
CGGTCCCCGCCGCTGGGAGGATGAGGTCCGGGCTGGACCCGGGAAGAGCCTCAGCTGGAGAGGA
CGTGGACGCGCCCGAGGCTCGGCTCCCTGGGCGGCTGCGGGGCTCCGGGCAGGAGGAGAGGGCG
GGCGAAGCAGAGGGAGCGGGCGGGAGTGGGGAGGGAGGAGTGCGGAGGGCGGAGGGCGTGCG
GGAGGGATCCCGGGAGTGCGCTTGTGTGTGTGTGCGCGCGCGCCCGCCCCGCGCGCTCCTCTCCC
CGCCCCTCGCCCCCTCCCTGCGAGCGCCCTCCTCTCCTCCTCCCCCCGCCCCCCGCGCTCCCGCCC
AGCCCCCGGAATCAGTGCCGCTGTTGCCGTGCAGGCTGCGGATTCCTCCAGTCCCTCCCTCGGCC
GCCTCTCCTCCCGGAGCGAGCGCGCAGCCCTGCGCAGCAGCGCCCACTGGTCCCGTCCTGTGAGC
CCCGGCCCCAGCCGCGGACAGACCCGCGGAGTCGCCTCCCGGCCCACCCGCCCGGCCGCCGAGG
AGCGGGAGGAGGACGGGACCCCGGCGCCCCCACCCCATCCCCGGGAGGTAGGTAGGGGGCCCGA
GGCAGGGAGATGCCAACTCACTGGGGCTGCCTCCCGGGTGGGCTCCGCGGCGGCGGACGGGCGG
GAGGAGGGGCGCGGGCCCAGGGCCGGAGGACGCCGCTGCCCGCCTGCCCTGCGCCCGGGGAGCC
GCGTGGGGGCAGGGAGGCTGCAAAGCCCGGGCGGGGGCGGCGCGGGGAGCCCGGGTCGCGGGT
GCTCATTGGTACTCGGATGATGCGGGACGAGGGGCGGCAGGCTCTGCGGTGAGGCGGCAGCAGA
CCCGCCGTGCGCACCGCTGCCGTCCGGCCCCAGCCCCGGCGCGGCGCGGCGATTCCTCCTCCCTG
CCTCCGCCGGCGCCTCCCTGCAGCCGGGCGCGGCGGCCCCTGACGCGGAGGCCCCTGGCGCGGA
GGTGGGTGCGGAGCGGACAGCGGACAGCCGGAGGGTCTATTTTCAGGTGCCCTCTCTGTGGCCCC
GCGGGTGGGGAGCGGGGGCGGCGGTGACAGGGCCGGGCTCTGCGCGTAGCGGTGCAGGGGATG
CGGCCAGGCGTGGTCCCGGCTGCGCCGCGCTGCAGCTGAGCCGGAGGCGGCGGCCCCGCGCCCC
CACCTCCGCCCCATCCCTGCGCTTGCTACCCTCGTGCGCCCGCCCCCCCCCCGCCCTTTCTGCCGT
CCCCACTCTCCCAGGGCTGGGCGTGAGCCGCCTCCGGGCGCCGAGCCGCCGCTGCGCCCG
CGI: 77 range = chr2: 73004709-73005568
CGCGTTCCCCCGCGGAGTGGCTCTCGAGTGCGGGGAGGTGTTGCGGAGGGGAGTGGACTTAGGG
AAGGGGCGGCAAAAGGGCAAAGGGAGAAATGGCGTGTGTGTGCGTGTCAAGGAATGGAGAGGG
CAGGGCGCTTGGGAGCAGGGCGCGAGGCCAGGCTCTGTTGGGCCCCGGCTCACGGCGCCCCTTCT
CTCTGTCTGTACCTGCGTGTGTTGCCGTCGGCGGCGGGGCCGCAGCCAGCGACGTGCCCCAGGAC
GGGCTGCTTCTGCACGGCCCCTTCGCACGCAAGCCCAAGCGGATCCGCACGGCCTTCTCGCCCTC
GCAGCTGCTGCGGCTGGAGCGCGCCTTCGAGAAGAACCACTACGTGGTGGGCGCCGAGCGGAAG
CAGCTGGCCGGCAGTCTCAGCCTCTCCGAGACGCAGGTAATCACCCCCGGTCGCGGCCTGCCCTG
CGCCCGGAGCCCGGGTGGAGGTGAGGGTGCGCGGGTGCAGGAGAGGCCCTGAGCCCGCCCCAGC
CCAGCCCTGCTGGGTTCCAAAAGGCCCCCATTCCCCGCGGCGCTGCGGTCAAGCCCGTCTTTAGA
GCCTCTTCCTCGAGACTGCGTGCAGCCTGCTGAGCCCGCAGGACTTTTGTCAAGCGCTAAAGACC
TAGCAGGAGGCAGAGTAAATGCAAACTGTATCCCGAGCCCGGCTCCCAAAGCTCCTCACGGGGG
GACCAGGTTCCCTGGAGGAAGCGGGTCGCCTCGGGAGCGGGCAGCGCAGGCAGCACCGAGGCCA
CTGGAGCTGGCTCCAGCCCTGGCATTCCTGCAGCCCTTTTCCCGCCACTGTGTCGGGGCGCTCATA
GTCCTGCGGGGAGCCGGTCCG
CGI: 221 range = chr2: 85213468-85216104
CGGGGGTCGCGTTGGAACCCCACAGGAAAAAGGCGCGGAAAGCCGCCGGGCATTTTCCGGGGTT
CCATAGATGTCCCCAGTGTCCTAGTCCGTGCATCAGCTCGCGCACTCGGAGGGACTCTAGGCAGG
GGGAGGGCCCCGCGGCCAGTATGTGCGTCCGAGGCTTTCCCGCAGGGGGCAGTGCCGCCCGCCC
GCGCGCCGATACGGTGGGAGGGGGTGGGAACCTGCGCGGAGTTCTGGAGGTTCTTTGGGAGAAA
GTTAGGGGATGCGGAGGGGTGGGCGCAAGACTTCCAGGACTCCAGGGAGGCCGTGGGGAGGGCC
GCCGAGGGTGCAGTGTGAGGCGCAGGAGGGGGTTGGGGGCGGTGCACGTTGCAGGGAGACGCA
GCCCCTGGAAGATGCGAGTGTGAACGTGTGAGTGTGAGTGCGTGTGTATGTGTGTGTGTGCGCGC
GCACCGCAGCTCTCCGGGTTCCGCGAGGCGCGCGGGTGTCAGCTTGCAGCCGGGGCTCCTCCCTC
CGGCCCCCCTGCCCAGCCCGGCGGTCCCTCCTCCCTCCCTCCCCGCTCGCCCCTCCCCGGCGGGCC
AGGGGCTGGGACGCCCCGGCGGAGCAGGCGGCGGCGGTGGCGAGTTGGGGAGCCCTAGGCTCGG
CGCTGCCGGAGGGGCCCGAGCCGAGCCGCCTGCGCCCCGGCCGGGCAGCGCCGGGCCCGCTTCC
CGCGGGGCCACGCCCTGTCAAACTTTGTTGCGGCGGCTAGCGCAGCGGGCCCGCAAGCGGGCGG
GAGGGGCGCCGGGCCGGGCCGGGCAGGGCGCGGGCGGCTAGGGGCTCCGAGAGCGGCGGCCCC
GGCCCGCGGCCCCACCATGCCCCAGCTCGGCGGCGGGGGCGGCGGCGGCGGCGGCGGCAGCGGG
GGAGGCGGCGGCTCCAGCGCCGGGGCGGCCGGCGGAGGGGACGACCTCGGGGCGAACGACGAG
CTGATCCCCTTCCAGGACGAGGGGGGCGAGGAGCAGGAGCCGAGCAGCGATAGCGCCTCGGCGC
AGCGGGACCTAGACGAGGTCAAGTCGTCCCTGGTCAACGAGTCGGAGAACCAGAGCAGCAGCTC
GGACTCGGAGGTAAGGAAGCACCGCGGCCACCCCCGGGGGATCCCGGCCCTGCGTCCGCTCACC
CGCTCTTGCCTTTGTGTCTCCTCCGCAGGCGGAGAGGCGCCCGCAGCCCGTCCGGGACACTTTCC
AGAAGCCGCGGGACTATTTCGCCGAAGGTATGTGCCCGCTGGGACAGCCCCCCACTCTCGATTCC
CGCTGCGCTCCGCTGCTCAGCCCGGGCGGCCCACCGTCCCCCTTGCTTGGGTGGACGCACCCTTG
CCCTCCGCCTTTATTGGCGGCAGCCCCCGTGGGGCGCGCGTGGGGGGCGCTGGGGTCCCCAGCTC
CCGCCTCGAGCCCCCTGCCGCGGCGCTGTCAGTCCCGGGGGCCTGGGCCTCACCTCGCCTTGGTC
TTGTTCGCAGTGAGAAGGCCTCAGGACAGCGCGTTCTTTAAAGGACCCCCGTACCCTGGGTACCC
CTTCCTGATGATCCCGGACCTGAGCAGCCCGTACCTCTCCAACGGACCCCTGTCTCCCGGAGGAG
CGCGCACCGTGAGTGCCCGTCGGGCGCGCCGGGGAGGGTGGGAGGCCGCGGCCCGCAGGATGCG
CCCCCGGGCTTGGCCATGGAGTGGGGGATGGGGCCTTCTGCGCCGATCCCAAGCAGAACTTGTTT
GCGGAGTTGAACTACTCTCTGGCGGCCGAGCGCGAGGCTGCGCTGGCCAGTGCCTGGATGAAAG
TAAAGTTACTTTAACTTTTCCCCTCTTGCGGGTTGAGGTTTTGGAGTCCACCTCTGGGATCTTCCTT
GGCCTCCAGAATTCTTCGCCTGCACCGAAGGAAACTTGGATTTGTGCCCGCTTTGGGGGGGTCTC
GCTTTCCTTCTTGGAAATCGGTCAGCTTTCTCTGGCAGTGGGGCAAGGGGCCTAGGGAGCTGGGT
TGGCGACGTTGTCCTCCGACTCCGGGTTCACTGGGCGGCTGCAGGCTGGTTCCTAAGAAACCCAG
TTTTCGTGGCGGGTTATTCACAGCCCCCGTTCCCACCCCCAGCCCTCGCACCGGGGCCTCAGCTTT
TCTGCGGAGCTAGCTCCGAATTTTAAACTCGCGTAGATGATTTCGAGGCGACCCAAGGCATTCTT
CAAGTTGAGGAACTTGGCTTTTTCCCCCTTTTGTCGCCTGCTTTTCTCATTTAAAGCGAGCGCTGC
GACACTTTAAACCTGTTAATGGGCGCGTATTGTGTGCTGCCCGCTGGCATTTAGAGCGGTGATTA
AGCAAATTGACCGGGCCCCAGACGACGTGCAAATGAGGGCGGATTCCTTCGCCCCCTCTCTCTGG
CTCTAAACTCCCGCCCCCCGCGTTGCGAGGGGCGCAGCTGGGGGCTGGCGAGGCCTTTGTGTCCC
CCAAGCCGCCACTCCACCCCTAGGCTCCCTGCCCAGGTGCTGGGTCCGATGACAGATGTTGGGTG
AACGCCTACCTACTGTGTGCCAGGTTCCCGCGTGCTGCTGCTTGGGCGCGATGTGAATCGTAGTA
GTTCTTTGCTTCCCGCCCAGATTCCCATTGCCTGCCACTTCGCTGTCGACG
CGI: 173 range = chr2: 119329502-119332035
CGAGGAGGCTGCAGGGACGCGCATGGAAGAGCCGGTGCGTGGGAGGGTTTGCGGGGGGGACAT
CGCGCCCCCTAGGGGTGACCCCAGTGGGTCCCGTGTGCTCTCCGCGGAGCCGGCGGAGCCTTGTC
CTCTGCACCCGGCGCGCAGCGGCCCCTTAAACAGTGGAACCGTGAGGCCGCTCTAAGCCGAAGG
GCTGGAATCTGGGTTTCTCGGGTTTTATTTTAGACCATTCGGCACCAAGCCCGAGCTCCCCCGCCG
CACCGCTTCCAGTCCCCTTTCTTTCCATAGAGCGACCCGAAGCCGGCGGTGGCGCAGGGAGCCGA
GTCTGATGAGCTCGCGGGCGGCTGAAGGCCGGCTTCCCTGTGGGGAACGCGCCACCTGTCGGCGC
CAGTGAGAACTGCGTCTGTGTGGCGCCCTCGGGGTATTCGGGGCTGCGGGGAGATGTGTGCCTGA
AGCCCTGCGCTTGCGGTGGGGACGTCCGGCCTCTTTCCTGGCAATTGACCCCTGAGGCGGGAGAG
ACAACGGAATTCCCACAAAGGGATCCTTCTCGGGATCTCCCCACCTCAAGACAGCTAAAGCTGGA
GGAAAAGCCCCTCCGGGGGGGGGGGGTGCGGGTTTGCCCTGCGATTCCGAAAGCAGAAAATAC
CCGAGCCACACAGGGACGGGCGCCGCGTTGGTAGTCGGGGCTACGTTCCTACTCCCTCTACCTCC
CCCGCGCTGTGTGACCCTGGGCGGAACCCCGCTGCTCTCTGGGCCTCAGTGTTCTTATTCGTAAAC
TGAGGGCGTTGGATGAGATTGGTCCTCTCCCAACTCTGACCTTGAAACTGATACTGAATCTGAGC
AGCGTCTGTAGACACCTGTGCCTTGCCTTCTATTTCTAGCCTTGAATAAATCCTGGACTTTTATGT
GCCATTTATATCCTAATCTCATATATATTTAATGTATAACTGCTGCCATTATTGTTTTCTCAATTGT
CTAGGTTTTCATTTGGATGGGGTTAGGATGGTCCAAATTATCCCGATAAGTGCCCATTAACTTAAA
CCTTTTTAAAAAATGAAACCAGTAAAACTTCATTCACTTTGCAGTGTGGACACTGCTGGAGAGCA
CCCATGTCGTGGGTCCAGCGAGGACACAAGGAGGGGCTTAGAGACATGCGGGAGGCTTAGATGA
GAAGACAGCACCCGGGCAGCGGTCAGTGTTAGAGAGAGGACCCGTAAGAAGGGCCGAGGCTAG
AGGGAGAGCGAAGACTGAGCCAACGACGCACCTGAGCCCTGGGGTGGGGGTGGAGACGTGGCTC
CTAACCCAAATCTCCCTGCCAGGCAGTGTCCGACGAGCATCGACGGCAGGCGTCGAGACCAGTG
CAGGGTAGCTCAGACCTCAAGCCACGCTTGACCTTTCCATGAAATGAATAAAACTCGAAAGCCAG
GGAAAGGGGACAGTACTTTGATCCGGAGATCGCTTATAACCTCTGCTTGGAGTTCCGAGTTCGTG
CGGCTCAAGGGAGGCTACAGTCCAGCAAGCTCTGGGCTCCAAGCGTGGGGACGGCAGCCCCCAA
GCTTGGCGCACCCCTCGGGAAGCCCCGGAACGGTCCTCGCCAGACATAGCCGGCTGTCCTGGTCC
TTAGCTTCAGGCTGGCGGCGCAAGGCCAGAGCGGCTGCCTTCTAGGCACCTGGGTGGAGGTCTCG
CATAGCATTCCCTGAGAAGCGAAACTGCCCTTGGGGCCGCAGCGAGCCTGCCACATCGAACTGG
AGACCCTCTGCTTTCGGGATAGATGGGACGTTTCTGCTCTGTCCTTCTTGGAGTCCCGGAATCGTT
CTGGGGCCGCGTGCTGCCTGGAGGCGGTGAATTTCAGGGTCTTGAGAAGCCGCGCACACACGGG
ATTCTGGGCGAGCGTCCCGTCTCTTAATTCCTATTAAGAGACGGGAAAATCGAGGGACTGGAGGT
CCCATCATTGTCGCGTGAGCAGCCTCCTGAACACCAAGCGAGACCTGAGGGTTCCGCTGGGGCCT
CGCCCTGACACCCGGGCCCTCCGTGTGGTCGAGAGTTTGCGCCCGCTCCCGCTAGGGCAGCGAGG
TCCCACTTGCGGCCGGCTGGGGCATGGTGGCACCGGTTGTCTACTCCCCACTTGTGACACCGACA
GCTTCCAACTCCTCAGACCCACCCCGTGGAATTCTGGACTTTGTGAGGGCCGCCGGGGTCCTGGC
CCTGGGGTCAGCTGCCATCTGACTAAGCCAGGACGGCGGAGCTCCAGGCCTTGCTCCAGCACTGC
CGGTGCGTCGGGGCCCGCGGAGAGCCCAGGGCGGGAGCTGTGGGCTGAGCCGGGTGGCCGCGTG
GACACAGATGCCCGGCCGGACTGAGCGGCAGCCAAGACTCTCCGTCCATCCCGCCGCTGGACTCG
ACTCTCCCAGACCCGCCACGGAACCCAGATTTGAGCACGCAAGATAAAGACGCCAGAGGCGAGT
GCGCGGCGGAGAACTGGCCGCGACACGGGAAGCTTCTGGGGCGCAGAACGCTGGCTCCGACTCG
CGCGGCG
CGI: 36 range = chr2: 172653235-172653630
CGACGGCGCGCTTGTGGCCCGGCCGGAGCTTGCGTGCGCGTTCTGACGGCTGGGTGCTGTGTTAC
AGGTCGGCGCAGTTCGAGCACACGGTTCTGATCACGTCGAGGGGCGCGCAGATCCTGACCAAAC
TACCCCATGAGGCCTGAGGAGCCGCCCGAAGGTCGCGGTGACCTGGTGCCTTTTTAAATAAATTG
CTGAAATTTGGCTGGAGAACTTTTAGAAGAAACAGGGAAATGACCGGTGGTGCGGTAACCTGCG
TGGCTCCTGATAGCGTTTGGAAGAACGCGGGGGAGACTGAAGAGCAACTGGGAACTCGGATCTG
AAGCCCTGCTGGGGTCGCGCGGCTTTGGAAAAACAAATCCTGGCCCTGGACTCGGTTTCCCAGCG
CGGTCAACG
CGI: 295 range = chr2: 174907710-174910885
CGCCCTCGCCCCCCCCCCGCAAGTATCCCCCACTACTCCCCCCCACCCCCGCGGCCCTAGCTAGCT
GACTTGACTGGCACGCGCCGGGAGCCCGGGCTCGGGCCCCTCGGAGCGTCTGATTGGCTGCGGG
GCAGCTCCGGTCTGCTCTGCCTGCGCCCTCATTGGGCGAGAGGCGCAGCCAGCGGCACTTCAAAG
CGGGTGCTCCTCGCACTTAGGCTGAGTTTAGCCGGCGGGAGCCTGGAGTCCGCTCGGCACGAGCG
CGGGGACGCGGGAGCCGCGCGGGACCCAAGCAGTTTTTCCGAGCAGCCGCCAGGCTCAGCCCCG
CTCCCAGCCTCGCTGCGCAGCCAGAGACCTGCTATGGCCACGTCTATACTCGGGGTAAGTCGCAA
GCGCGGCAACGCATTTGCTTGTTTTAACCGGAGTAATTTTTCGTTATGGCTTCTGGGGTCTGCGGC
TCCGGAGAAACTGTTGCTGCTAGACTGCAGCTTCAGCTTCTATTAGCACTTTCCACATTTCTGGGA
CTGATTTTTCCCCAAGTTTTTGGAACCCAGAAAGATGCCTTTGCAAGAAAAGGCCTTCTTAATCAA
GTCCTTTTTCATTTGGTTTCAAAAAAAAGCTAGAGGTTTCTTTTCTTCCCCTCGTGATTTATACCCC
ATCCCCCGCATTGCTTTGGGGATTTTGTTGCAATTATGCGACAATGGTGTTTCCAGAAAACAGTCT
TAATCGTTTTGCAGTCCATCTAGTCACGCTAATAAACATTTACATTTCCGCGGCTCCCCAATTCGC
CCATGACTTTGAAATCTGTCTCTGGCCGCTTATCCTCGGATTATTGTTTTCATAATCGCTGTTTTTG
TGTGTATGTAAACTTTAAAACCAAACGTTTTCTATTAAAAAAGAAAAGTCAAGTCGTAGCCACCC
TGGGAGAGCTGCAAGTTTCGCCCGTCCCTGGCAGCTGTTTCGGCCGCCTCCGCCCTCACCCGGTGT
TTGTTGTCTCTGGTCTCTGTGTTTACCTTCGCGCTTCACCCCGAAGGGCGACTTGGGGTCTTCTGG
GAGATGAGCCGGTTTACGGCGACAACCCAAGTCCGGTTGTAGGCAGGGTTGCAGCGGGGAACAC
GGAGGCATCTTTGATTTATAGCCTCAGCGAAGTGCAACTTTCCGGCGCCTGGACCTTCGCCCGGG
TGATCGCCCCTGCTCCGCGCCCGGTGCGGCGACTCCTCCGCGGGCGCGTGGGGAGGAGGCGCGA
GGCGGGGAGCCAGGACCCCCCGGGAGCAGCCGCAGGGGACGAGGCTGCTCACGGGTGCACTTTG
TTTCTTCCCCCACTTCCTCTTGCCCTCCTCCTTCTTGCTCCCTCCCCCATCCCACCCACTCTAGGAA
GAGCCGCGCTTCGGAACGACCCCGTTGGCCATGCTGGCGGCGACCTGCAACAAGATCGGCAACA
CGAGCCCGCTGACGACGCTGCCAGAGTCGAGCGCCTTCGCCAAAGGCGGCTTTCACCCCTGGAAG
CGCTCCTCGTCCAGCTGCAACCTCGGCTCCAGCCTCTCGGGCTTCGCGGTGGCCACCGGGGGCCG
TGGCTCGGGCGGCCTGGCGGGCGGCTCGGGCGCCGCCAACAGCGCCTTCTGCCTGGCCTCCACGT
CGCCCACGTCGTCCGCCTTCAGCAGCGACTACGGCGGCCTCTTCTCCAACTCGGCGGCTGCCGCG
GCGGCAGCGGCCGGGGTGTCCCCGCAGGAGGCGGGTGGCCAGTCGGCCTTCATTTCCAAGGTGC
ACACGACGGCAGCCGACGGGCTGTACCCGCGCGTGGGCATGGCGCACCCGTACGAGTCCTGGTA
CAAGTCGGGCTTCCATTCGACGCTGGCGGCCGGCGAGGTGACCAACGGCGCGGCGTCGTCGTGGT
GGGACGTGCACAGCAGCCCGGGCTCGTGGCTGGAAGTGCAGAACCCCGCTGGGGGGCTCCAGAG
CTCGCTGCACTCGGGCGCCCCCCAGGCCTCGCTGCACTCGCAGCTGGGCACCTACAACCCCGACT
TCAGCTCGCTCACGCACTCCGCCTTCAGCTCCACGGGCCTCGGCTCCTCCGCCGCCGCCGCCTCCC
ACCTGCTCTCCACCAGCCAGCACCTGCTGGCCCAGGACGGCTTCAAGCCGGTGTTGCCCTCCTATT
CGGACTCCAGCGCCGCCGTGGCAGCCGCCGCCGCCAGCGCCATGATATCGGGCGCCGCGGCTGC
CGCCGCCGGGGGGAGCTCGGCACGCTCTGCCCGCCGCTACTCGGGCCGCGCCACCTGCGACTGCC
CCAACTGCCAGGAGGCGGAGCGGCTGGGCCCGGCCGGGGCGAGCCTGCGGCGCAAGGGCCTGCA
CAGCTGCCACATTCCGGGCTGCGGCAAGGTGTACGGGAAGACGTCGCACCTGAAGGCGCACCTG
CGCTGGCACACGGGCGAGCGGCCCTTCGTGTGCAACTGGCTCTTCTGCGGCAAGCGCTTCACGCG
CTCGGACGAGCTGCAGCGGCATCTGCGGACTCACACGGGCGAGAAGCGCTTCGCCTGTCCGGTGT
GCAACAAGCGCTTCATGCGCAGCGACCACCTGAGCAAACACATTAAGACGCACAACGGGGGCGG
CGGGGGCAAAAAGGGCAGCGACAGTGACACGGACGCCAGCAACCTGGAGACGCCCCGTTCCGA
ATCCCCCGACCTCATCCTGCATGACTCCGGCGTCAGTGCCGCCCGGGCGGCGGCAGCGGCGGCGG
CGGCAGCGGCGGCGGCGGCGGCGGCGGCCTCCGCGGGAGGCAAGGAAGCAGCGTCTGGCCCCA
ACGACTCTTAGAGGCCGGGCGAGAGGCGCGAGCACACAAGCGAGTAGAGACACCGAGAACGAA
CGAGAGGTTCGGAGGGCGAGCGAGCGGGAGGCGGGAGGGCAGGGGCTTCAGTGACGCCCCCAG
GGCCCGGGCTGGGCGCGAGGTGGAGCCGCTCAGGGCTCCCGGGCTGCGGTTCGCCCGCTGTGCG
AGGAGCTCCCCTCTGCCTTCCGCGCCCGGATAAGAATCGAACGCGTGGTCCGGAAACAAAAGCG
AACCATCCTCCGACACAAACACTTTAAAAACTGTACTCCCAGACGTACACATACACCGGAGACCT
ACAACCACAAGCACGCACACTCGCGCGCGCACACACATACCACTCGCCCAAACTTCTCGAGCG
CGI: 289 range-chr2: 176652334-176656692
CGAAAAGGAGGCGGCGCAACGGCCACCCCTTCCAGCACCTCGGCTTTGTCCTTCCCGGGGAAGGC
GGCCACATCCCTACCCGCCTTGCTCCTGAACGTAGTAAACAATCTCACAAACAACCACCGCTGCC
CACGCTCTCCATCCGTCCTCCCGGCCTTATCAGACCTCCGTTCTCCCGCACTCTTCGGGCAGGGTC
CCTAATAAGCTCAGGCTGAAAGAACGTTTGCCACCTCCCCCACCCTCGTTGAAAGAAAAGGAAG
AAAAACAGCAGCAGCGAGAAACCTCCGGGGCGACTCCTCCCCCGCCCCCAAGCACCAGCGCACA
GCATCCCCCTCTGTCTTTGTTGTGGTTCTCCGTTGCTTCGGGCCACGCCGTTCAGCCAAGCAACCC
GGACCTGAGAGTGCACAGCCAGGACTAGCTTAGGGGGCGAGGGGTTGGTCTTTGGGAAACCAAG
CGCTCAGGACAGAGGTGGAAAGTGGGTCCCGGGAGCCAGAAAAGAGAGAGAAGGGCAGACGGC
TGGGTGGCAAATACAAAAATAGAAATAATTTAGGGGGATGCCCGCCAGGCTTTTGCGCCTGCTCC
TTCTCCCCCAATTCGGAGCAGGTTCCCTTCGGCCTCCCGCGCCCCGGGGCGCCCCCTGGCGGCAG
CGGCAGCAGCGGGCAACGCGCGGAGGGCTCAGGGGGCGCACAGGGGACTCCCGGGCACACTCA
GAGAGGCGGGCGCGGCCCCCTGGCGGTGGCGACGTAGTTATCTGGTGAGCGGAGCCTCGTCCCTC
TGGTCCGGCGGGCTCACGGCCGTCTTACTAAGCACCGCGGCCGAGTAGGGCAGGAAGCCGCTCTC
GGAACGCGGCGCCGCCGCGCTGCAGCCGAAGTCCGAGCCTCCCCCGGCCCCGGCGCCCCCGCCG
CCGCCGCCACCACCACCACCGCCGCCGCCACCGCCACCCCGGGAACCCAGGGCTGCGGCAGCTG
CTGCCGCGGCTGCCGCCGCCGACTGACTGCTGTGGCAACTGAGGCACGAGCAGGGTGCAGAGCC
GCCGCTGGGGGGCGCGCCGGCCGCCGCCGCCGAGGAGGCCGCAGCCGCTGCGGCTGCCGCGGCT
GCCGCGGCAGAGGCCGCGCTGTTGAGCCCCGCGGCGGCCGCGGGAGCCTGGTAGAGACCAGGGT
GGCGGAAGCTACACAGCAGCTCCGGCCGAGAGTAGGGGTGCGAGAGGGCGCGGAAGGTGTCCA
GTGGCCGGATGGAAGTAGCGAAGGGCGACGAAGCCGCGGCCGCCGCGCCTGAGGCTGCAGCCGC
GGCCGCCGCCGCCGTGACGCCCACGTGCGGGTAGTAGTGCAGCGGCACGTGCGAGTGGAAGGGG
TAGGGCAGGCTTCCGGTGGCGGCCGCGTGCGTCATCATGTAGGTGTAGAAGCTGGGGTCGGCTGG
GTGCGGCCAGGACATGGCCAGGCGCTGCCGCTTGTCCTTCATGCGCCGGTTCTGGAACCACACCT
GCGGGGAGAGACGCGCCGCAGCCTGGGTTAGGGAGCGCCCCGTGTTCCCAGCTCCTGTCCCAGG
ACCTCTGCCCCTTCCGGACCTCTGAATGGCTTGGTCTACTTCTCTCCGACCAAGCCCAACCCCGAG
TACCCTGTGGTCTCCCAGCTGGGAAAGTGTGGACGGCAGTGTGTGGACCGCCGTGGGCACACCGT
CCTCAACGAAGAGGGTCCTCTCCCCCGCGTCCGGCTGCTGCTGCTCCTCAGGCTTTTATTCCCTTA
CTTCTTGCTGCACTTTTTTGTCCCAATCCAACCTTTCCTCTCCCTCCCGCCACCCACCAGTGCCGGT
CTCGCTGAGCACCCGTCTCTCAATCCCAGGATTTGTACGGGGATTCTGGGCAGCCTTTGAAGAGA
GGCTGCCGCTCAGCTTTTCTGAGAGGTCGCCGCGCGAAGTCTTGAGCCCTCCGAACTGCAAGGAC
CTGCCCCTAGGGGCACGGGTCCGATTTTGATATTGAAGGGATGATTTTGTTGGAATCGTTTGCCTT
AAATGAGTGGGTAGAGCAATGTCTCCATAAACGGGGAAGGGGACGTTTCCACCCCTCCCAACAC
TATCTAATAAAGACATCATTCGTCACAACTCTAAATTAAAGAAAGCCCGATCAAACCAGAGGGA
GACTTCCACACTCCTCCCCTACCCCGTGGAGATTTTTTCTTTTTTCTGCAGTGTCGAACGCTCCATG
AAGGGCTCACCAAATCGCCTAACCTCCCGGCTATCTCTCCCAGACGTATAATAAAATTAATAACC
TAAAGTTATATATAAATAAGGACAATTTCGTTGCATTTTTCCCCGCAGGAGGTTGCCCTTTTTTCG
TTGCCCAAGAGGAAAATGTTCAGGAAACTACTGTCTCAAACCAATCGATTTTAAAGATACAGTAT
CCTTTTCTCCGTGTAACGATTTATGCGGAAAATAAATCTCCAAGCTCAAGAGCAAATGAAAAGTT
TCACCTCTGGTTCCTGCTTGAGGAACAAAGACCAACTGGGCTTGCCGCCTAGGGGAAAGTGGGGC
CGTGGGTATGGGCGAGGGGGCATCTGGCCAGGCGTTGGGCACAATGGAGCAGGGGCGAGTGCTT
TCAGCATTGGAGTCACCATTCGGGGGCCTTCTTAGATCCGTCAGGCCGGACAACCGTTCGGATTC
GGTGGCCGGGAAATAAATAAGCCAATTCCTTTGGTGACTACCCCCCGCGGATTTCCAGACCCTTA
GCTAAATCTAGCCACCCAGAAAGGGGAAAGGGGAAAAAGAAACAATCAACCCAGATGCCCCCG
GGGAGGCCAGAGCAGGCATGCACTGGAATTGATACCTTGATGGTGGTTTCGGGCAGGTTGAGTG
CCGCGGCCAGCTCGCACCGGCGGGGCCGCGACACATAGTTCTCCCGGTAGAACTCCTTCTCCAGG
CGCGCGATCTGCTCGCGGGTGAACGCCGTACGGTAGCGCCGCACTTGATCCGCGCCAGAGCCGG
AGCCACCCAGCGCCGCGCTCCCGCCGCTGCCTCCGCTGCCTCCATGCAGGCTTCCGAGGCCTGAG
CCCGACGCCGACGTCGTGGTGCCGGCAGCCGAGCCGCTCTCTGCGTACCCTGGCAAACAAACGAC
CAACAGCGCATGAGTGGCTGTAGGACCAACAGCCCGGCGCTGGCGCTGCGCGCGGATCGGGGAA
GCCCCGTCAGGAAGGAGAGTCGCTGCCGGAATTGATGGGGTCTGTCATGCTTACAAATTGCTGCC
GTTAATGGAATCAATAAAGTTTGGGGAGCCCTTCATAAGCAAATAATAGAAACGGAATTAGGAG
ATTTCTTTTTTAATAATTAGAAATTTTCAACCAAGGAGGAAAAGTGGCCGAGGGAAAATGCCTAC
CTCTGGGCGCAGTTTGGGAAGCTCTGGGTTTCCCATGGTCTGGAGACCGCAGGGCAGCTTTTTAT
ACGGCCTCTAACCTTTATCTGAGTCTTCGGGGTTTCAAATATTTTAAGAGTTCCAACACAGCAGTA
GCTCAAACCCAAGCCAATAGGGGGTGAAATATACTTTTCAACTCTTTCTCCCGCTGACCTAAACA
GAGACGCCGGCGAGGCTTCCTCCGACTTACCCAGAAGAATGCCCCCTCCAGCCCTGAGGGCACA
GGGGCAGGGGAGTTCAGAGTAGTTGCCCAGGGTACCTTTCCCAAAAGCAACTCGGCTGCTGACTA
GGGGTCTACCGGCTCGCTCGGGCTCTTAGACACGAGCGCAGAAACATTTTCTCGGACTCAGGAGC
GAGGGCGGGTGGGCCTGGTGTTCCAGGCTCCGCAGGCCTGAGCCTGGCGGGAAAGCTCAAGGAG
CAGAACTGGAAGGCACGGTCCCAGACATGCGTCCCGCCCCCGCCCGTGCTAGCTCGGTGTAGCTT
GCCTGTGGAGGGTCTGAGAGGGGAAAAGGCACCGGGAAAGGCTGGCGGGGGCCGCGGAGGAGC
AAAGAGGATGGGACTGGAGAGCGCGGTGCGGCCGGCGCGGTTACCTTTGCCATTGTTTTCCTTAA
GCTGAGCGGCGCCGAGGCCCCCGGGGGAGCGAAGCGCGGAGCAGCCCACCTCCACGTCGCTGCT
CATGTCGGCCTCAGCGGCCGCCTCTGAATAATGGCCCGGCTTCTTGCGGCTCTCGGCGGCGGAGG
AGATTTCGGAGGAGACGGTGCTTTCGCTGCCCGTGTGCTGCAGGTTGAACAAAGTGTCTATTTCG
AATTTGCCCTTGGCGGGGAGTTCTCCCAGAGCGCTGTGCAGGGGGGCAGACGGCAGGCGCGGGC
TTAGGCGAGCCGGGTGCTGCGAATTTTCCAGGGCCTCGAGCACAGCATTGCCAGCCGAGTTGGAC
AAATTGGAGAATCTCTTGCCCGCCG
CGI: 25 range-chr2: 176720618-176720921
CGCCGCTGTAAGTACCTTCCCGCTTCTTCCGAATCGCGAAGAGACGGTACTGATCGCGGGGAAGT
GGTGGGCTAAGGGGCCAAGCCAGCGGAGTGGGCGGATAGAAGAAAGGCCCTGCCTCCCCCACCC
CACCCCCACCCTCCACCCCCGGCCTCGCTCTGCGAGGTTAGCCCGCTTCGGGAGGTGGAGGCGCT
AAATCGAGGTCCCGGAAAGCTTTTCTGGGGCCTGGTTCACAGACACCTGCCTGAGAGAACGGTCC
GCGGGGTCTCGCCCTTGTCTCCTTGGGCCTCCCTAGACCTTCGCG
CGI: 20 range = chr2: 236438623-236438823
CGAATCCAGTCCCGGCCGGCGCTTTGAAGCCTGTGCTGTGCGATTTTCTCAGCAGTGAGGTAGGA
ATAGACGCGGCTAATGACAGGAAGGTCGCGCGGGTGAGCTGACCTGTGTGTGGCGCAGCCTTGG
GTTCCGCAAATAGGGCACCCACAGTAACACGTGTGGCGCCGACCCCGCCGTGCGCAATCGGGGC
TTTATACG
CGI: 22 range = chr3: 11721578-11721824
CGATGGGACCGCGGCTTGACACGCATTAAGGAGCCACTGAAAACTCCATCTAAGCAGAAAAGCG
GCTTAGTCATCCATGCGTTCTGGGCAAGTTATCCTGGCCGCACCACGTGGAACGGAGTAGATAGA
GCTCGGGAAGCTGAGCGCAGGGCGGACAACGGGAAGGCCAGAGCCGTGGGCAAGCACGCGGAG
GGCAGCGAGGGGCACTGGCACCGGAGCCGCGGGGAGGCAGGAAAACGCAGAGGCG
CGI: 68 range = chr3: 12983649-12984210
CGCTCCTGTCCCAAGGGCGCGTCTCTCTCTCCCACGCACAAACTCGGGTCCTGTTTGCTCTTCTCT
CACCCAAACCCACAGTCTGGCGGACGCACCCCGCCCCGCAGGCAGCTTTCTCTCGCACGCCGGGC
GCCCGGTCCCGACGGTCACCCGCACTCCCGCACAGGCATCCCCACAGCCTGCGCCAGCCCACCGC
TCAGCACCCGGGAAGGCCGCTCGCCCCGCGCCGCGCCCTCACCCGCTGTCAAGCTCTAGCTCCAG
AAGGGACTGGGTCCTCTCCGAGTTGCAGTGCAGGCACCACATGGCGGCCGCAGTGGGAGCGGGC
CGGGCCGGGAGCCGGGACCCAGGCCCAGCAGAGGCCGCCGACTCCCGCCAGCGAGCCGCCGGAT
CCCGGGGCCGACACCCGGCCACCCGGAGACCCGACCACCCGCCACGCGATCACGTCGGGGCTCC
TGGTCCAGCGTCCGCCGGCTCCCGCGGCTCCGGCCCCAAGTCCGAGCCCCAGGCCAGCCAAGCCC
GCCCCTCCGCCGCCGCCCGCTTGGCGCAGCGCGAGGCCGGGCCG
CGI: 120 range = chr3: 25680561-25681574
CGCGGGGAGCGGGCTGGAGGATTCTGCAAGCACAGGCCCCTATGGAGCGCCCGTTCGGGGGACG
GGATTTCCCTCCCTTTCCCCTCCCCCGCCCGTTCGGAATTCCGCCCCCGCCGCGGGCCCGGAACAA
TGCAGCCGCCCGTCCCGGGGACCAGCCACTTACCACCCAGGTCAGTGCCCCGTTGCCGCCGCCCA
CGCCGGCTCCCGCGCCGCAGCCACCCGACTTGGCCATGGCGAGTGCCTCCAGCTCACAGGCCCTG
AGGCCGCAGCCGCCGCTCCCGCCTCCCTGCGGGCCGCTGGGCCCCGCCGCTCCGCACCCACCGCT
CCACTCGCCGCACTCCTAGCCGCGCCGACCCCCGCGCCCCATCGCGAAGATCCGGAGCGGACGTC
CAGCCGAGCCCGCTGAGGAGGCCGCGCCGCCGGCTGCCCTCAAACTCGAGGCGCGGCGTCCGCG
TCGCCCGGGCCTAGCGCGGCGGCTGAGGAGAAAGCAGGGAGCGACCGGCGGCGGCCGAGCGGC
GGCGTTGCCTTCTCGCCCGCCCGCGGGCGCCGCTGCAGGCCGGGCTGAAGCCCGGGCGTGCGAGC
CGCGAGGGCGGCCGGGGAGCCCGAGGCGCTCGGACGTGGCGAGGACGCAGAGGTGCCTTGTCCT
TCTCACACTCCGCGAAGGCCAGCCACTCGAGTCGCCAGAGTAGTCGTCCCGGTCGCCGCCGCTGC
TTCAAAGGCAGCCTTAGCCTCGCTGCAGCCCCGATTTCCTCACACACACACACCGAGAGGGACAA
TAAACAGAGCCGCCGCCGCCGCCGCCACGGTCACCTCCCTCTTGTCCGGCATAACACCGCACACA
CACATTTGCACACCGGGGAGAGAGGGAGGGCGCGGCGGCCGCCCCCGCTGTCAGTCAGCGCCAT
GCTGTCCAATCCGGGCCTTGTCGCCACAGCCTCTCGGCCAATCCCAAAATTCCAAGCCCCCTGGA
GGGGCGGGACCCGAGGGGGGAAACGTCATTGCGGGTAGCAACCG
CGI: 23 range = chr3: 148613030-148613264
GACCGGCGCTTCGCTAACAGCAGCGACCGCAAGAAGCACATGCACGTGCACACGAGCGACAAGC
CCTATCTTTGCAAGATGTGCGACAAGTCCTACACGCATCCCAGTTCGCTGCGCAAACACATGAAG
GTAATCGCCGCACTCTCGTCGCCCCCTTTGAGGCAGGAGCTCTCTTGGCTCTCGGCTTGGGGTCGG
GCGGCGAGTGGCAGACAGGCGGTGGCGGGAGCCAGAGGAA
CGI: 41 range = chr4: 4910534-4911092
CGGGCAAACCTCCGGAGGCCCCGGTGCACCGCGCGTCCAGCCGGCCCAACTCGAGCTAGAAGCC
CCAACCACTGCCCAGTGCCTGAGTTGCAGTCTTGGGTCCTTTAGAAACCTGGAGATGTGCGTAAA
ATTCAGATGCCGGTATTCCCGAACTTCCCCAGGCCTCAGCATATCTCGGCGGCCTGTGGACAGAT
GGGAGGCTACCAATCGCTCCGGCGTCCGCAGCCCGACCCCTGCCGCCAGACCCCGGACGTCTTCC
GGATAATAAAGTTCCCGCTCTAATTCATTTTCCCTAATCTGGACGCCCCTAATCTACAGCTTTTAT
TGCGCCCAGTTAAAAGTCGAGGGAATTCGCTGTCCCTCCGCGCTCGGATAATTACCCCTAAATGG
CCACGGCAGCCCCTTGTGTTTCCTGGAGATTAGAACCCCGCAGTCATCAATGGCAGGGCCGAGTG
AGCCGCCAATCACCTCCGCTCACTCCCTGAGAGCCGCTGGCCTGGGCCGCAGGAGGAGAGGCCA
TAAAGCGACAGGCGCAGAAAATGGCCAAGCCCCGACCCCG
CGI: 300 range = chr4: 30330303-30333940
CGAGCGCGGCGGCTGGCTGACGCTCCCTCCCGCCTCCTTATCCCGGGCCCTCCTCCCTCGCTCTCT
CCTAGTGTGTAGTTTCGGCGGCTCGGCTGCTACTGCCGCCGCCGCTGCAGCAGCAGCGTCAGGGA
CAAGCCTGATCCGTGAGCGAGCGAGCACCCAGCAGCAACCACCACTTTGGGCAACTTGCGGGTTT
CCTGCTCGCGGGTAGCGAACGGGAGATTCCCACTAGCGGTTCAGTGTCTCCGAGCCGCTGCAGCC
CGAGGGGCGGCGGGAGCGCCCGCACCAGACTCTCCCAGACGCGAAAAGGGACGAGACAACTGC
GGAATTCACCAGCCGTGCCAGGGCACTTCCGAGGCCACAACCGACTGACACTTTTTCTCCCCTTG
GCAAACTGGATTTTTTTTTTTAAGCTACTTGGCAGCTGTCTCTGACTCCACCTCCCCCTCCACCCCC
GCCCGAAAGCCTTTGGCTTTTCTTCGGAAATCCAGACAGAATTGGGCATCTTTGTTAATTGCCGTT
GGGGACGCCCGGCCGTGCGCTTTCGCCGGCTAACGTCGCCTGTGCTCCGAGCCTGGTTTGCTCAC
CTTTGAACTGCAAAGGGATCAAGTTCAGCTTGAGTTCCCTGCATTGGGAAGGAGAGAGAGCGTGC
AAGAGAGCGAGTGGGAGAGGGGGAAAGGGGAAAGGAGAGGAGGGAGGAGAGAGAGGAGGGAG
GAGAGAGAAAGGGAGGGAGGGAGGGGAGGGATCGAGAGAGAGCGGGGAGAGAGAGGCTGCAA
TCTCCTCCCTGAATCGCGCACAGCGCTGCAGATCCCACTGCTCCGACATGCGGGCCGAATGCAGG
TGAGAAAAGGCACGGACTCTGCGGCTGCGAACCCAAACTTGGGCACCGCACGGTGCGCACTGCT
CAGCCTTCGCCCCCGTGGGCGAAAGGCTGCTGCGGTTTCAGGCGGCTGCTTCGTGACTAATGACC
TTGCGCAGAGTTGTTAAGAAAAAAGAGAAACCCGCGCTCTCCGGGGTGAGAAGGGACTGACTCT
GGGCGTCTCTGAAGATGGCTCGGGCTTCTCTTTGGCGCGCCGGGGGGACCCTGACACTGACCGCT
CTGTGACGCGAGTAGTCTCCCCTGCACCGTGCCCGAAGCGACGTGCCGGGGGATTTTTCATTCTC
GATCTGTTGACTGGCTCCCCCGCTGCATGAGCAGAGTCGGAGTTGAGACTGGCTTGTTGCTGGCC
CCAGCGCCTGGTGCAGGAAGCGACTCACGTTTGTCTGGGTGGCCGGAGCCGGAGCAGAGCCTGG
GTTTGGAGTGAGTGCCTGGAACGTGAATTGGACTCAACTCGAGTAGCAGCAAAGACCAGCGGGC
TGGCAGGCGGGGGAGGCTGCAGGCTCATTCCCCACCTCTTCCCAGCCCCACTGCCCGTCTGCCGG
AGCGGTTCTGGCCCCTTCCGACAGAGCGGGGACTAGAGCCGGGGATTCTCCGCCCGCTGAGGGG
ATGACTCTGGGTTGGGGGAGCGCCGAACCCGCGGCGCGCAGTGTCCCGTGAACTGTGAGTACTGC
GACTGAACGGCGGCAGGCGAGCGGGCGATTAGCACCCATTGCATGAATTATGAAACAATAACTT
TCGGAAGAAGCAGGAGGAAAAAAAGAAGCATCTATCGCTGCCCTCCCACCCCCATTCCCGGCCA
ACTCTCCACGCCGCTTTTGCCCCCTCCCTCCCCTCCCTCTCGCTCCTTCCTTTCCGGGAGAGGGGA
GAGGACTCGGGGGAGGGCAGGCGGCCGGCCCCGGAGGAGGGGGGCGCCGAGGGGGCTGTGGTT
AGAAGGAGCAGTAGCAGCAGCAGCAGGAGAAGATGCTGAGGATGCGGACCGCGGGATGGGCGC
GCGGCTGGTGCTTGGGCTGCTGCCTCCTCCTGCCGCTCTCGCTCAGCCTGGCGGCCGCCAAGCAG
CTCCTCCGGTACCGGCTGGCCGAGGAGGGCCCCGCCGACGTCCGCATCGGCAACGTGGCTTCAGA
CCTGGGCATCGTGACCGGATCGGGTGAGGTGACTTTCAGCCTGGAGTCCGGTTCCGAGTACCTGA
AGATCGACAACCTCACTGGCGAGCTGAGCACGAGCGAGCGGCGCATCGACCGCGAGAAGCTGCC
CCAGTGTCAGATGATCTTCGACGAGAACGAGTGCTTCCTGGACTTCGAGGTGTCGGTGATCGGGC
CCTCGCAGAGCTGGGTGGACCTGTTTGAGGGTCAGGTCATCGTGCTTGACATCAACGACAACACG
CCCACCTTCCCGTCGCCCGTGCTCACGCTCACGGTGGAGGAGAATCGGCCGGTGGGCACACTTTA
CCTGCTGCCCACAGCCACCGACCGCGACTTCGGCCGCAACGGCATCGAGCGCTACGAGCTGCTCC
AGGAGCCCGGAGGCGGCGGCAGCGGCGGCGAGAGCCGGCGCGCCGGGGCGGCCGACAGCGCCC
CCTACCCCGGGGGCGGCGGGAACGGCGCGAGCGGCGGCGGCTCGGGAGGCTCCAAGCGGCGGCT
GGACGCATCAGAGGGCGGCGGCGGCACCAACCCCGGCGGCCGCAGCAGCGTGTTCGAGCTGCAG
GTGGCGGACACCCCGGACGGCGAGAAGCAGCCGCAGCTGATCGTGAAGGGGGCGCTGGACCGCG
AGCAGCGCGACTCCTACGAGCTGACCCTGCGAGTGCGCGACGGCGGCGACCCGCCTCGCTCCTCG
CAGGCCATCCTACGGGTCCTCATCACCGACGTGAACGACAACAGCCCCCGCTTCGAGAAGAGCGT
GTACGAGGCCGACTTGGCTGAGAACAGCGCCCCGGGGACCCCCATCCTGCAACTGCGCGCAGCC
GACTTGGACGTGGGGGTCAACGGGCAGATCGAATACGTGTTCGGGGCGGCCACCGAGTCGGTGA
GGCGGCTGCTGCGCCTTGACGAGACGTCCGGCTGGCTCAGCGTCCTGCACCGGATCGACCGCGAG
GAGGTGAACCAGCTGCGCTTCACGGTCATGGCCCGCGACCGCGGGCAGCCCCCCAAGACCGACA
AGGCCACCGTGGTCCTTAACATCAAAGACGAGAACGACAACGTGCCGTCCATTGAAATCCGCAA
GATTGGGCGCATCCCCCTCAAGGACGGGGTGGCCAACGTGGCCGAGGACGTTCTGGTCGACACC
CCCATCGCTCTGGTGCAGGTGTCCGACCGAGACCAAGGCGAGAACGGGGTGGTCACCTGCACCG
TGGTGGGCGACGTGCCCTTCCAGCTCAAGCCAGCCAGCGACACCGAGGGCGACCAGAACAAGAA
AAAGTACTTCTTGCACACCTCGACCCCTCTGGACTATGAGGCCACCCGGGAGTTCAACGTGGTCA
TCGTGGCGGTGGACTCAGGCAGCCCCAGCCTCTCGAGCAACAACTCCCTGATTGTCAAGGTGGGA
GACACCAACGACAACCCGCCCATGTTCGGCCAGTCGGTGGTGGAGGTTTACTTCCCTGAGAACAA
CATCCCGGGCGAGAGGGTGGCCACGGTGCTGGCGACAGACGCAGACAGCGGTAAGAACGCCGA
GATCGCCTACTCGCTGGACTCCTCTGTGATGGGGATCTTTGCCATCGATCCCGATTCTGGGGACAT
CCTGGTCAATACCGTGCTGGACCGCG
CGI: 21 range-chr4: 41678680-41678940
CGTCACCAGTTACTCCCGTTTAGCCAACGAGCTGCGTGTGAGCTGCATGGAGCGGAAAAAGGTCC
AAATTCGGAGCTTGGATCCCTCCTCTTTGGCGAGCGACCGATTTAACTTCATTCTGGCGAGTACCA
ACAGCGACCAGCTCTTCGTAGTGAACCAGGTCGAAGTCGAAGGCTCCAAGTACGGCATCATCAG
CCTGCGAACTCTGAAGATCCCTTCGTTCCACGTGTACGTGCTCAGAAACCTCTACGTCCCCAACCG
CGI: 217 range-chr4: 85636684-85639823
CGCAACTTCCCGCAGTTACTGCCGCTCAGCCGCACTAAGGAGGTCCGGAGACTTGGAAGAAACTT
CGGAGACTCGCTGAAAGGCGAAGAACTGAGCGAGAAAAGGACCCCAGGCTGGGCCCTGAAGTCC
ACCTGGAAGGCTGGGAGCCTCCGCGGCGCTGCGGCCTACTACCCCGAAGACTAGCGTGATCTTGG
GGCAACTCAGTTTGCCTCAAGCAAGGTCTTTGCGGGATGGTACATTTTCCCCAGGCCGTGTCCCTT
CATCTCACTCTTCTCCTGGCTCGCTCTTGTGTTAGGGTGTTGAGCTCCACGATGTCCCCAAATGTG
CCCGGACGCAGCGTGTGCCAGCCCCCAGAGTGGAGGCAGGTGGGAAAGCGCGGGCCCAGCGCTG
GTGAGCGGCTGGAAGCGCTCGCCCTGCCCTGGGGACGCTGGACTGTCTTCAATGGGCTCACCCAG
GCCGAGTGTACTCGGGCGGCGAGGGCGAAGAGGTGAGGTGGGGACACCCCAGGCGGAGACGTG
CATGCAAATACACTCACTTCCCGGGCGGTGGAGGGCTCACCGCTGCCGGGCCTCACTGCTTCGAA
GAGAGGCAGCGGGTTAGGCTGGCGAAGGCGGAATGGGGCGCTGGACGACAGGGAGGAGCCGGG
AAAGCAGAGATGCAGTGGCCTCTCCTCCCCTCTCCTCCCCATCTTTCTAATCCAGGCCAGGCCTGC
GGTCCTGGCCAGGCCGTTTCAGGAACAGCCAGGGCCTCGGACGAGACACACGTCCCTCACCGCGT
TGTGGTCCACACGAACACACACACACCGGCTCAGCCCAGGCGTACTCAAGACTTAGAGAGAGGG
AGGCGCTTGGATACAGCCACGCGAGCTCAGAAAAGCGAGAATCCCTTTCTGGAAGCCCTGGCCC
AACCTAACTGGTGTGATTCCGGCGCTGTCAAACTACTGGGGGGGGCCACAGGATGGACTGAGCG
GCATGCACACCAGGGGCCGCGACCCGGGAGTGGGCAGAACAGGCACGGCAGGCAGGCATCGGG
GCGCGGGTGGTAGTACTCACGAGGGGTACAGGCCAGGCGTGCGTCCCTCCAGGGCGGGCTCTGC
ATCACTCCGGGCCAGAAGATGGGCGTCCGGCCAGGCAGCTCAGCCAGCGGCTTGGGGTACCGGC
CCACGGCGGCCACGGCCGCGGCGCTGGGGCTGAAGTAGAGCCCGGGCGGCGGCGGCGGCGGGCT
CAGGCTGCTAAAGCGTGGCAGTCCGGCCAGCAGCCCCGCCGGGGATGAGGCGGCGGCTGCGGCC
GCGGCAGCAGCCGCGGCGGCGGCAGAGGCGGAGGAGGCAGAGGCGGACGAGGAAGAGGAGGA
GGAGGAACCGGAGGGCGAGGCGGAGGGCAGGGCGGCCCCCGAGGCCACGGGCATGGAGGGCCG
GCTCAGGATATCGTTGATGCCGTGTGGGGTGGCGGCCGAGAGCTGCTGCGGGGGGCTGCCGAGG
GATGAGAGCCCCCCCGTGGCCGGGGGCTTCAGGCCGCCTGGGTTGTGGGTGCCCAGAGGCGGGG
AGGGCGACGAGGAGGACGACGACGAGGACGAGGAGGAGGGGGGGCCGGCAGGCAGCGGGGGA
TACGCGGCAGGGTACAGCGGGGTCTTCATCTCGGCCATGCTGTGCAGGGCGGCCAGGGGAGGGC
TGCTGAGCAGGAATGCGCTCTGCCGGGTGCCCTCCATTGCCCCCACCGCTAACATCCCACGGCCA
CGCCGGAGACCGTAGCCTTGCAGCGAGGGCGCTGGCTGGTGCCCCCCGCGGGGCTCAGAGGAGC
CGGAAGCGCCGAGGGCGCGAGCGGAGAGGCACTCGGCGCGCCCGGAGGCGAGCTGCCAACTGA
ACCAAAAATGCCGCTGCCGGGAGTTGCTCGCCTAGCTGCGCAGCAGAGATGTCCAAACCCTCCAC
GCGGGAGGCGGCAGCTCGCCGAGAAAAGCAGGCGTCCCGGCGGGCTAGGCAGTCCTTTCGTTCC
GCGAGTCCTAGATTCGATCCCTGGCTATTCTCTCTTCCTCACTTTTCCTAGCTGTTCAAAGTGCGCT
ACTTCTCATCTTCTGCCCCCGGGAAATAAGCAAAACAAAACCCAGGCTGGCTCCGGAGAGTTTGT
AGCAAAGTTAGTTGCCGAATCTCCACTTTGAAGTTGGAGGGTGGGGGTGGCTTGCTTTCTTTGGG
GAGGTTCAAAGGACGCCTTGTGCAGCCCGTGGCGCTCCTCTGATCTTACTGGGATGCTCTGCTCTT
TCGGTCGCGCGGCTGATTCGCATTCGACGCTCACTGTGCCCGGGGAGAAAGCGCATCCAGCCCGC
GGGAGATCTAGCCTCTGTGCGGGCTTCCTCCGCCCACGCTGCCCCGGGCTGCTGCGCCAGAAAGG
GCAGTGCCACGGATCCCCGCGGCTCCCAGGTCCTCACCTCCACCCGCCTTGCCCTCTGGCCCACG
GGGCACTGGAGTTGCAATATTGAAAAGAAAAAGGGGGAGGAAAAACACAGAAAAACAAAAGAC
TAAGTGTGAAAAGTCTGACGGCTGGGTTTCGGCGCCGCTCGTCAGTCCACTTCTGCAAACGGGCC
CGGCGACCCCCGCCCCACCCCCGCTCCCTCTCTCCCTCTCACTCTCAGCCTTTGACTCTCCTCTCTG
GCATTTTCTTCGCGGCTTCCCAGGCTTCGGTCTTCTAAGTCCTGTCCTCTGGCCAAACTGACCCTCT
CCGCGGCTCCTCCGCTCTTTCTCTCCTTTCCTCTCTCCCTCCGGGCCTGACAGTTCAGATGAAGCTC
TCAAAGGTAATAAAACATTTGCATCACTCCCTACCCCTCTTTAGCTGGGGGACGAAAGGGAGGGG
GAAGTGGGGGACCAAAATGAGAACTTTGAGGGACGTCACTCCGAGGCTGCGGGGGCCGGCGAGC
CGGGCGCAGGCCGGGGGCGTAACCACCGTGTCCAATAGCTCGCCGCCTCGATCCTTGCGTCGCCC
CTGGGGCTCGCCCACTCGGGGGCGCCGCACCCTCTGATTGGCTGAGGCGGGCCAGGCGTAGGGC
CGCGCCCGCCTAGTCCCCTCCCGCTCCCTACTTCTTTCTCCTGTGGGCGGGGCCAGGAGGGGCGG
GAGCTGGTGAGTGGTGCGGTTCCGCGCTGGGGCCAGTGGCCG
CGI: 39 range-chr4: 94974810-94975333
CGCGGTGCGGAGCGTCTGGAGCGGAGCACGCGCTGTCAGCTGGTGAGCGCACTCTCCTTTCAGGC
AGCTCCCCGGGGAGCTGTGCGGCCACATTTAACACCATCATCACCCCTCCCCGGCCTCCTCAACC
TCGGCCTCCTCCTCGTCGACAGCCTTCCTTGGCCCCCCACCAGCAGAGCTCACAGTAGCGAGCGT
CTCTCGCCGTCTCCCGCACTCGGCCGGGGCCCCTCTCCTCCCCCAGCTGCGCAGCGGGAGCCGCC
ACTGCCCACTGCACCTCCCAGCAACCAGCCCAGCACGCAAAGAAGCTGCGCAAAGTTAAAGCCA
AGCAATGCCAAGGGGAGGGGAAGCTGGAGGCGGCAGAGGCGGCGGCGGGAGAGCTGTGGGGTT
GTTCAGGAAAAGTTGGGCTGGGGGCTGAGGCACCTGAAGCAGTGAACCAGGACTAGGTAGGAAG
CAAGAGCATCCTCCTGCGCAAGCGGAGACGCAAACGCGCTCCTGAACCCATGCAACCGCCCGGC
CCAGCGCCG
CGI: 162 range = chr4: 134291113-134293078
CGGAGCCAGACCTGACGGTGGAAATCTCTGAGAGCGCCACGCCAGGCACTCGCTTCCCCTTGGAG
AGCGCATTCGACCCAGACGTGGGCACCAACTCCTTGCGCGACTACGAGATCACCCCCAACAGCTA
CTTCTCCCTGGACGTGCAGACCCAGGGGGATGGCAACCGATTCGCTGAGCTGGTGCTGGAGAAGC
CACTGGACCGAGAGCAGCAAGCGGTGCACCGCTACGTGCTGACCGCGGTGGACGGAGGAGGTGG
GGGAGGAGTAGGAGAAGGAGGGGGAGGTGGCGGGGGAGCAGGCCTGCCCCCCCAGCAGCAGCG
CACCGGCACGGCCCTACTCACCATCCGAGTGCTGGACTCCAATGACAATGTGCCCGCTTTCGACC
AACCCGTCTACACTGTGTCCCTACCAGAGAACTCTCCCCCAGGCACTCTCGTGATCCAGCTCAAC
GCCACCGACCCGGACGAGGGCCAGAACGGTGAGGTCGTGTACTCCTTCAGCAGCCACATTTCGCC
CCGGGCGCGGGAGCTTTTCGGACTCTCGCCGCGCACTGGCAGACTGGAGGTAAGCGGCGAGTTG
GACTATGAAGAGAGCCCAGTGTACCAAGTGTACGTGCAAGCCAAGGACCTGGGCCCCAACGCCG
TGCCTGCGCACTGCAAGGTGCTAGTGCGAGTACTGGATGCTAATGACAACGCGCCAGAGATCAG
CTTCAGCACCGTGAAGGAAGCGGTGAGTGAGGGCGCGGCGCCCGGCACTGTGGTGGCCCTTTTCA
GCGTGACTGACCGCGACTCAGAGGAGAATGGGCAGGTGCAGTGCGAGCTACTGGGAGACGTGCC
TTTCCGCCTCAAGTCTTCCTTTAAGAATTACTACACCATCGTTACCGAAGCCCCCCTGGACCGAGA
GGCGGGGGACTCCTACACCCTGACTGTAGTGGCTCGGGACCGGGGCGAGCCTGCGCTCTCCACCA
GTAAGTCGATCCAGGTACAAGTGTCGGATGTGAACGACAACGCGCCGCGTTTCAGCCAGCCGGTC
TACGACGTGTATGTGACTGAAAACAACGTGCCTGGCGCCTACATCTACGCGGTGAGCGCCACCGA
CCGGGATGAGGGCGCCAACGCCCAGCTTGCCTACTCTATCCTCGAGTGCCAGATCCAGGGCATGA
GCGTCTTCACCTACGTTTCTATCAACTCTGAGAACGGCTACTTGTACGCCCTGCGCTCCTTCGACT
ATGAGCAGCTGAAGGACTTCAGTTTTCAGGTGGAAGCCCGGGACGCTGGCAGCCCCCAGGCGCT
GGCTGGTAACGCCACTGTCAACATCCTCATAGTGGATCAAAATGACAACGCCCCTGCCATCGTGG
CGCCTCTACCAGGGCGCAACGGGACTCCAGCGCGTGAGGTGCTGCCCCGCTCGGCGGAGCCGGG
TTACCTGCTCACCCGCGTGGCCGCCGTGGACGCGGACGACGGCGAGAACGCCCGGCTCACTTACA
GCATCGTGCGTGGCAACGAAATGAACCTCTTTCGCATGGACTGGCGCACCGGGGAGCTGCGCAC
AGCACGCCGAGTCCCGGCCAAGCGCGACCCCCAGCGGCCTTATGAGCTGGTGATCGAGGTGCGC
GACCATGGGCAGCCGCCCCTTTCCTCCACCGCCACCCTGGTGGTTCAGCTGGTGGATGGCGCCGT
GGAGCCCCAGGGCGGGGGCGGGAGCGGAGGCGGAGGGTCAGGAGAGCACCAGCGCCCCAGTCG
CTCTGGCGGCGGGGAAACCTCGCTAGACCTCACCCTCATCCTCATCATCGCGTTGGGCTCGGTGT
CCTTCATCTTCCTGCTGGCCATGATCGTGCTGGCCGTGCGTTGCCAAAAAGAGAAGAAGCTCAAC
ATCTATACTTGTCTGGCCAGCGATTGCTGCCTCTGCTGCTGCTGCTGCGGTGGCGGAGGTTCGACC
TGCTGTGGCCGCCAAGCCCGGGCGCG
CGI: 308 range = chr4: 149582663-149586506
CGGATTACGCAGCCACTTCTCCCTGCAAAGTGCACGCTCCCGCGCCGCGGCCGCACGCCGAGCAC
GGCAAGCGGTGCCCCGCCGCGCGCCCCTACACACCTGCCTCGGCCGGGGTCCGTCTCTCGCCGTC
TACCTGTTGCAGCGCTTGCCACCGCCACGAAACCCCTGCTGCGGAGCCCCCCTAAATCCAACCAC
ATCCAGGCCAGCGAGTGCCGCTCTCCCTGCAGCGGGAGAGCCCGAGGCAGCAACGCTCTTGCAC
CCAGGTGACTGACTGCACGGGCTTCCTCTGAGCAGCCTGAAGTTGGCTCCCGTCTCCCGGGCCCA
ATGACACCCCGGGCGCGGAGGGGCTGAGAGGAGGGGGCAGGGGCAGGGGCAGGGCCAGGGCGG
GCAAAAAAGGGAGGGAGAGAGGGCGGGCGAAAGCCCGGAGGGGGAGTGGGCCAGCTGGAGGG
CTGGCCCTTTTAAAAGCGGGTCACATGTCCAGATAAAGTTCAGGTTGCTATCCCGCCCCCCTGAA
CCCTTCCTATTGGCCCGAAGAGAAGCAAGGCTCCACGCCGCACGGGTCAGGCGGGCTTCTTATTG
GACAATCCAGGCTGTCAGTCACCAGGCGGGCGACATGACTGATACAAAGTTGCTGCGACACAGC
CTGGACTCGGCAGCTTCCTTAAAGCCGCAGCCGCGGCGGGAGCTTGGGGTGCCGGGCGGTGGCT
ACATCAGGCGGCCTCCGCACGCTGCCCCCACCCCCATCGCTCGGCCGCCCCAAACTTGCTTACGT
CCACCCTGCTCTCCTTCTGACCGACCCCAGGATATCTGGAGAGATGTGAAAAGTGTGCTGACGCG
GAAGAAGTCGGTTTTTCCGCGAGCAAAGTGTCGTCCCCTCACGCTCTCCACCTGCAGCTCCAGGT
GAGCTGCAGCCCAGACCCTGGCGCCGCCCAACAGCGTCCGCGCGCGGGGAGGACTCTGGGTGGG
CTTCACCCGACCCTCGAGGTTCCTCTCCCAGCCCCTTTCCACTGTCTCCAGAGTTACCTCTCAGCA
GAGCAGCAGCAGCCTTCGCTGCCAGCGAGCAAGACGATCACGTTCACTCGATTTGAATTTAGTTG
TTTTAACCGTGCTGGAAAAACCCTTTAAACACAGCTTTCCTCCCATCCCTTAGACCCCTAACACCA
AAAAAAAAAAAAAAAAAAAAAAAAAAAAAAAGAGAGAGAGAGAAAGATAAATGAAAGAAGTG
GCAGGGTCAAGAGCTGCTCCTCTACTTCCTTATAATTTTGTCACTGTACTTACTACTTTAGTATTG
GATTTTTCCACTAGTTCAATTTGAAAGCAAGCTTACCATCGAGGATATTCACGTTATAATGGGTGG
AGGGGGTGGGTCACATAAGTGGGAGAAACAGACAGGAGGGGGGAAGGAACCAAACTGTTTTCT
AAGGATCGGTTGTCTGCATGAAGAAATTTCGGTTTCCCTCCAACCGCCCCCTCCTCGATTCTCCTC
CCGCCCACCCAAATGCTGCAGTTACTAAGCAAAAGGATGGGGTTCCTAACTTTGATTTCTAAGGA
CTTTTAGGCTTCTCATGTAAGGCCCCGTTTTAGGCTCCCCGCTGGCCCGGAGTCAGCATCAGTGAC
GTATACACCTAAATCTGGTCCCCCGGCCCTCGGCGACCCGCAGCCCGGCGGCGGCAGCTGTGCGG
CCTCCCGCCTCACCCTCGCTTCGCGCCCGCCAAAGCCCGAGGCGGGGGACCGGGGGACACCCTGT
GCCCAAAGGCCCCTCCTCTCCTTAACAGTTAGTTAAGCTCCGGGATTCCGGACCCCTAACCCAGG
GCTGAGACTTGAACTCTCAGCCGCTCCAGTCAGGAACTCCCTGGAGATAGGGGCGACAGCGGCA
GCGCCGCGAGCTGGTCCTGACCCCAGACTCTAATGCCCCTCGGTCCCCAGCTTGGAGCTGCCCCC
GGCGTCCCCGCAGCCAGGAGTCCCCGCCGAGCGTCCCAACTTTCCCGCGGGGCCCACCGTCACGC
GCACTCTCCCGGCCCAGCGAACTGGGCATCGATCTCACGTCGGCAGAGCGCGGGCGGCGGGCGG
GAGCAAGGGGAGTCCCGGACCTAGAGCCTGGGCACGCGAGGCGGCGGCGGCTCCGCAAGCGTTG
CCGAATTATTCATTATTAAAGGAATGGACGTGTCTCTTTTACTAAGTTTTTATCTCCTTTGAGGAG
GGCCGGTCTTGCCCTGGATCTCAGCTTCTTGCAAAATCTAGGTTAGAACCGTGCAGGGGCAGCCG
CCGCGATCACTCGCCCGCCACCCAGCACCCTTGCCCCAGGGCCGCCAGCGGGCAACTCGTCTCCT
GCCCGCATCCCACCTCCGCTCGCCAACCCGCGCCCTCTGCCCTGGGCGCGCAACCTGCCTTGCCC
GGGGCCGCCCCTGCGCTCTCCGGACCCCCTCTCGCCGCTGTCAGCGCAAAGTGACTGTCGCTGCA
CTCACCTTTTCTAGGACATGGTGGGGAGGCTGGGGCGGGGGAAGGGGAACGGCTAGACTCCCGC
CGCCGCTGCTGCCGCCGCCACCAGCAAGTCCAATATTGGCTGATCGGCGGTGAGGATGGAGAGG
ATGATAATCCCGGCAGTGGCAGTCGCCCTGCTGACGGTGGGTAGAGCAATAGTGCTGTTACCCTA
CAAGAGGCGGCGAGGCAGCGGCGCCAAATCGCGCAGAGGGGGACGCGGGCACCCGCCCGCCCC
CAGCGGCGGCGGCCGGGCCTCTGGCGGGCCCCTCTCCCGGGGCTGCGCGTCCGCTCGGCGTTGAC
AGCGGCGTCCCTGGGAGCCGGGAAGTCCGGCAGGAGGATCCCGCGCCCGCCGCTCCGCAGCGCC
ACTCTCCGCGCCCCCTCCCCGGGCCCTGGGTGGCCGGTGACAGCTCCGGCGGCCGAGCGCGTGTG
TGAGGGGGCGGTCGCAGGGGGAGCCTGCGCCCCCCTCCCCGGGTCACGCCCTCACGAGCGGGAG
GAGGCGGCACCGCGTCCGGCCACCCGCGGGTGGGAAACGGGCAGGGGCGCCGGCAGCCGCCCTC
CCACTACGGCCACTGACAACCTGGAGGCCAAAGGAAAACTGAGCCCAAATGCACGCACCCCCTT
CAGCCCTGCCCGCAGCCCTCCAAAGTCCGGCCCTGACCTCGCGCCCGGCTTCAGGTGCAGTTCCC
ACCTGCGGGGGGAGGCGAGGGCTGGGGACCTCGCGCGGGGGGCGGGAGGGAAAAGTTTTCTTTT
CCTGCTCCTCGGCTGCCCACCCCGCCAGGCTGCTCCTCTCCCCGCCCCTCTCGGGCTCGCCGCGCC
TCTGCCCCACCCCCGCCGTGGGGGGGCGCCGAGGAGGGAGCGGGCGTCTGTGCGCGGAGGCGTG
CGGGGAGCTGGCTGGGGCGCTGCGGTCGCGACCGGTCCCCTCGCTCTGACGGCGGCTCAGACGCC
CGCGGAGGCGGCTCGGCGGGGCAAGCCGAGGAAGGGGCCGAGAGCCGGGACCCCTCCTCCTCCG
CTCGCGGCGCGCCGCGCGCCCCCGCGTCTCCTGGCGCGCCCCCGCGCCCTCGTGCCCGCTCGGGC
AGACACCTACGCAGACGCGAAAGTGAAAGGGGGGCGACTGGGCCCGGCAGGCGGGCGGCCGGC
GGCGCGCGGGCACGCGGCAGGGGACAGAGGATGGATGGGTGAACCCGGATCCCGGGATTCTCCC
GCGCCCGCTGCCCCTCTGCGGAGCCTCCCCGGAGTCCCACGCCCCCACCCCGGCCACGGAGATCA
CCACGTCAGGAGATATGTATGCGTGTGCATGTGTGTGCGCGCGTGTTCACGCGCGTGTGCGAGGT
ACGCTTCTGCCCCAGACTCTTCCCCCGCACCG
CGI: 44 range = chr4: 154436043-154436665
CGAGAAGCTGAACGAGCTGGCCGACCAGGACTTCCCCTTGCACCCGCGGGAGAACGACCAGCTG
GATTTCATCGTGGAAACCGAGGGGCTGAAGAAGTCCATCCACAACCTCGGGACGATCTTAACCAC
CAACGCCGTTGCCTCAGAGACAGTGGCCACGGGCGAGGGGCTGCGGCAGACCATCATCGGGCAG
CCCATGTCCGTCACCATCACCACCAAGGACAAAGACGGTGAGCTGTGCAAAACCGGCAACGCCT
ACCTCACCGCCGAACTGAGCACCCCCGACGGGAGCGTGGCAGACGGGGAGATCCTGGACAACAA
GAACGGCACCTATGAGTTTTTGTACACTGTCCAGAAGGAAGGGGACTTTACCCTGTCTCTGAGAC
TCTATGACCAGCACATCCGAGGCAGCCCGTTTAAGCTGAAAGTGATCCGATCCGCTGATGTGTCT
CCCACCACAGAAGGCGTGAAGAGGCGCGTTAAGTCCCCGGGGAGCGGCCACGTCAAGCAGAAAG
CTGTGAAAAGACCCGCAAGCATGTACAGCACTGGAAAACGAAAAGAGAATCCCATCGAAGACGA
TTTGATCTTTCGAGTGGGTAAGGAGAGGGCTTCTGTGCCCGACG
CGI: 122 range = chr4: 174686622-174688044
CGGCGGGTACGTTGGGGATGCACTCGCGCAGTTCGGCGAAGGCGCTGTTGATGCTCTGAGTCCTG
CGCCGCTCCTTGCGGTTGGCGGTGCCTCGGCGCTTCACCGGGCGCGGCCCCCCCAGGCCCGGGGG
CCCGGCGCCCGGCGGCACCCCCCCGTAATGGGAGTGGTCCAGGCCGGCGGCGCCGCTGGCATAC
TCGGGGCTGTAGGACAGGGCCATGCTGTAGTCGGGGGGCGACATCTCGGGGTGGCCGATGAGCC
AGCCATGGAAGTAGGGGTTCTCCTCATGGCTGCAGCGGCTGGCGGCGGCGGCGGCAGCTGCGGC
GGCGGCGGCGGCAAACGGGTAGCCCTCGTGGTGCACCACCGGGTGGTGGGGAAAACCACCTACC
AGACTCATTTCGCCCTCCGCGCCCCTCCACGCGCCCCAGCGTGCGCGCAGCCCCGCCGCGCCCTC
GGCCCGGGCCCCTGCCTCAGCGCTCGGCGTCCTCCCCCACCCCCCACCCCCCAGCCCCCGGGCGC
CCGGGCCCGCCCGGCAGCCGCAGAGGGGGCTGCTGCAGCCCGGGCCCCGTCCCCCCGCCTGGCC
AGCCGGGCCCGCCTCAGCAGCGCTGCGGCCGCCGGCTCCCCATGGGGCGCGGCGAGCTGGTCCT
GGCACCGTGCGCCCCTGGCCGCCGCCGCCGCCGCCTCCGGTTCCCGCCTTGCTCCACGGCCCGCG
CTTCGGCTCCTGCTTCCCGGGCTGCTGCGCGGAGGCAGAATCCTCTCGTGCTCATACAAAGGTGC
CGGGGCTCCCGCGAGGCTGGTACGCGGAGTCTCGGGAATCCAAGCCCGGGCCGCGGTCCTGGCA
CCGAAGCTAACCCGGAATCCAGGGGCGAGTCTGAGCCGCGGCTGGAGGAGCCGGTCCAGCTGTG
CCGGGGGGCGGCGGGGTCGACTGCTCACTGCAAAGCTACCTCCGTGCGACCCTCCCCTTCCCGCC
TCTTCCCCTCCCCCCACCAGCCCGATCTGGGTTCTTGGGCGCTTATTGTTTTAATGAAATTTTTGGT
TGTTGTTGTTTTGGTCCTTAAATGTGATTTTAGCTGCGAGTAACGTGTCCTCGCTCCTCTCGCGCTC
TCTCGGAGGGTTTTCAGAGAGGTCTCAGTGCATCCATTTTCTCAGATCCTCTCCTTTCGGGGCAAG
GATCTGGGGCCCTGAATGAGCCTTGGAGCTCGAAGACCGCGGCTCGGGCTCTCGGAGGGCTCTGC
GCGGTGGCCGAGGAAGCTCCGGGGTGACGGCGGTGGTTCTCCTGGGCTGGACGACGTGAGGGGA
GCAAGCGGATTTTCCCAGCAAGATCTCCATGTACAGCTACATCTTTAGGGCCGCTCGGGTTAATA
TATGTCGTCAGAGGCTCCTCACGCCAATCCCGGGGCGGCAGGGGCGGCGCCTCGAGCTCTCG
CGI: 245 range-chr4: 184255825-184258373
CGAGATTGGCCGCAGATGCCCCGACGGGATGGCCTGGGGAGCCGAAAGCTGTCCCCAGCCCCGG
ACAGCTACTGCCCACGCCGCGCAGGCCAGCCTCACACAGTAGCGAATCTCCTTCACGCGACTTCC
TCGCAGGCCTGTGAACCCGCAGGGGAGGCGGAGGTCGGTTCAAACCCTGCGAGAGCCCCTGCCC
AGCGCGGAGTCCTGCACACGGTGGAAAGTCCTTACGCATTAGCGGGCAGGATTAAACGACTTCA
CACCACGAGGTGCAAGACTGGGCTTCGGGGCCTTCCTCGGTGTTGCTATTTTAAAAGAAGCTGCT
CAGTAACCCAGAAGAAGATGACGGGGGACGGTGGTGTAGACCCGGCTGGAGAGGAGTCCAGGG
CCTGGGAGGCGCCCCCTCTGACTGAGGGGCTGGGGACCCTGAAGCTTGGCGTCCAGGCCCCGCCA
GCGCTGCACCCGGCATTGTCCCGCCTGGGGCAGGCGTCCGGTCCCTCGGCAGTGCGGGCCAGCAC
GTACGTAGTAGCTGCTGAATAAATAAATGCCTATGCAACAGGGAACGTCCCGGCCCACCCCGGCC
GGGCCAGGGCCTAGGAAGGGCGGGCGTCCGGCGCCGCCGTGGGTCCCTCCGGCGTGGCCGCCCA
CACCTCCTGGCCACGGCGGGGCTGCGGTGGGGGGACATCCTGGGGCGGGCGGCTGGACGGAAGG
GACTAGAGTCGGGGCGCGTCCACGGCCAACCGCCCCGAACCAAGCAGGGGAGGCCAGGGACCA
GGAGCTTCCGGGGCCCCGCGGCTGGAGCGCCCGGACGCGAAGGGGCGGCGGGTGACGGGGCGC
GGTGCGGGCGGCTACAGGGCCGGACGGGAGTTCCTCCGCTTTCCGCCGCGCGGCTCGCGGTCTCA
GCCGAGGTCATCGAGGGCCAGGAGCCCTGGCGGAGGCCCCGCCGTCCCGGACCCACCGACGCAC
TCCCAGGGCTTGGCGCTTTCCCACCACCCCGGACCGCCCTTGGGGTGCCGGAGGGGCTCGGGGCG
CGGCGTGGGGAGCCTTTGTGCCCTCTCCGCTTGTTGAGGTTTTTTCTCCGGAGGAGAGCTCCCCGC
CCGCCACCCCTTTCCTCTCGAAAGTTTTGTGGTTTCCCTAGAAGACGAAAGAGGTTGAGAAGGTT
GCGGAGACTTTTGCAATCTTCCCCGTCCAGAAACTCTAAAACCTCTCGGCGTTTGGCCCAGGGGA
GCTATCTGGGGTCAAGGGGAGGGAGCTGGCCGCACATTCCTTCGCCCGCCTTCCTCCCACCTGGA
GCCGCGGCCCCGCGGGGCAGTGCGTCCGAGCTGGTGCGGCGACGCCGGCAGCGGCGGGGTTAAG
CTCAGGAATGCGGCGCGAGGAATGCGCATGCAGATGAGGCGGGCGGGCGCATGCTAATCGCATG
CAAATGAGGGGCCCGCCGCTGCCGCGGCAGCGCCGCAGCTTCCCGGATCCGAGCCCAGACGGCG
GCGGCTCCGGCAGCCTGGGCGCCGCGACCCTCGGCGGCCAGAGGGGGACGGGGCGGAGGAGGA
GGAAGAGGCGGAGGCAGCGGCGGCGAGGGGGAGGAGGGTTCGCTCGCCGGCTCCGACGCAGAC
ATGGTGGACTGATCCGCAGCGGGGCCGCGAACAGCGTTTCCTGAGGCACCTCCCGCGCGTGGTTC
CGCCGCGCCCCGCGCCCTGCGCCCCTCAGCCCCTCGCCGGCGCCCGCGTCGCGGGTTGGCAGCCT
AGCCCGGCAGCCGCGTTCCCGCCGCGTCCCGCGCCCGGTACCTATGGAGGCGCCGCTCGCCGGCG
AGGCCGCCGACCATGCCTAGGAGGGCCGGGAGCGGTCAGCTGCCGCTGCCCCGGGGCTGGGAGG
AGGCCAGGGACTACGACGGCAAGGTCTTCTACATTGACCACAACACCAGGAGGACCAGCTGGAT
CGACCCCCGGGACAGGTGGGCGCCGGCCGCGGGGGCGCGGGCCCGTTCGGACACGGCGGCTGTT
GTCCCGGAGACCCGGCCGCGCGGGGGGCTGGGGCGGCGCCGGCCCGCGGTGCCCCGAGGGGTCC
CGGGAGGGATGTGGGGCTGGCTGCTCCGGCGGGGCCGAGGAGCCGCCCGTACCCGGGCTCCTCC
GCCCACCGGCTTCCCGACGCCCCTCCGGGGACCCGGCCCCGAGGCAAGGCTGGAGTCGCTGCCCC
GGGCCGACGCCGCGCGCCAGGCTAACGGACCCGAGGGGCCACCTGAGCCCTGTTAGGAACTTCC
CTAGGCTTCGAGGTGCCCACGTGGAGGCTGCAGGACACCTTCGGCCTCTGGGCGGCGGGGGTGG
GCTAGGGCCTCACTGCCAGTTGGGGAACGAGCCCTCCGGGCCACCTGTGGCATCCCTCAGGCCGT
CCTTGGCGCGGGAGCCTGGGCTGCGAGCTCTTGGCGGGGGCGCCCAGACTGGCCGGAGGTCCCG
AGCTGGGAAGGGGCTCCCGCCCCCCGCCGGGCCCTGCTAGTGGGTGTGACTGACTTTGCCCCTCA
GAGTTTAAAAACGCGCGTTTGGGGGTTTCGCTGTCTGTCG
CGI: 33 range = chr5: 360287-360592
CGTGCGCCTCAGAAGGGGCGTTAGGCAGAACGCGTGCCCATTCTCAGGTGTGCTCGGCGTGTGTG
TGCTCGGCGTGTGTGTGCTCGGCGTGTGTGTGCACACGTGTGCCGTGCGCCTCAGAAGGGGCGTT
AGGCAGAACGCGTGCCCATTCTCAGGTGTGCTCGGCGTGTGTGTGCTCGGCGTGTGTGTGCACAC
GTGTGCCGTGCGCCTCAGAAGGGGCGTTAGGCAGAACGCGTGCCCATTCTCAGGTGCGCTCAGCG
TGTGTGTGCTTGACGCGTGTGCACACACACATGTGACGGTGTGCCG
CGI: 18 range = chr5: 1161803-1162051
CGACCGGCGTGCACCGTCTCGCCATTCCCAAGGGCAGTGCAGAGCATGGCACCCAGCACCCGAC
AGGCGAGGACGGCCCAGGACAAAGCGGCAGCTCTAGCTGTCCTCAGATTTTGGGAGGGGGCTCC
CAACACGGACACGGTAGGTAGTTGGAGCAGGAGGGACGGGCAGGCCACTGACCACGCAACCGG
GCCTCTGTCAGCGGCCAGGGGCAGAGGGAGGCAGGACCGCCCCTGGACAACCGACACG
CGI: 584 range = chr5: 3647468-3656054
CGAGGCTCCGTGAAGAGAGGGAAGCGAGGCAGGGGGTGAAGGGACCGCCTGGCCGGTGTCCAG
GATACGGGTGGCTCAAACCACCAGCAGAACAGCATGCCGACTCTGGCCCGATGGCCGCCTTCATC
CCGTAGCCCCAACCCGGCCTAAAGCCGAGAAGACGCCAGAGTGCGCTGCTGAAATTCCCGCGGA
GTCCGGTGTGCTGGAGAGCCGCAGCGGGGTGAACTCCCGGCCCCGTCTCTGCGTAGGAGGTGGTT
CGCAAAGTGGCCCCGGGAGCCGGGACTGGTACCCGGTTCCCCACGGCACCGTCCGGAGCTCTCCA
GCCACGAGGCGCAGAAGTGGCCTGCCAGCGCCTTCCCAGGCTCAGGGAGGCGAGGAGGCCCGTG
CACTTGGCATCTTCTCCCGGGAGCCGCACGGCCAGGGCCGCGGCGAAACGGAGCCCATCTCAAGT
GCGCCGCGCCTGGCCGCGTCCTGTAGCCCGACGAGGCTGAGGATGGAGAGGGGAGACGCAGGGA
GAAGAGGGACGCGGGACTTGGACCCAAGAGGCCGCTCCGTTGCCGGTCTGGCGGCGGCGCCGAC
TCGGGTTCGCGCGTTCCACACAAGTTTCCTGTCTGCCTCTGCACACCTGGTGGACAAACCGGGCG
TCCAGGCCACACCGTCTTCCCCCTTCGCGGGGGCGCGGGGGATGTTTCCCTCCGGCTGCCAGGGG
GCTTTCTGGGTGAAGAGAAAGCCCCTCCCCCCGCGTCTCCCCCACCCCTCCCTCCGAGAACCCGC
GGCGCCGACTGCGCCTGCTTCCCCCGAGCTGGCGACTTCTCCGCGGGATTTGCCCTCGCTCAAAG
TTTGCACAATTGAAAGAGCCCGCAGAGCTCGGCCGCTCCCCGCTTCCCCAAGGGCGGCGAGGCCG
GTCATTGGCAGACGATCGGTTACTACCCAGTAGGGGCCCACGGGAACCCGCATCTGGAGTCGGG
GGTGTCACGCCACGCCGGTTCAGTGGCTCGCGGAGAGCGTCCGGGTGCACTTCTGCCAAAGATGT
CCCCTGGAGGCCCCGGCCGCGCGGGACTCGGGGGAGAGGCCGCTCCCCCCTCGCTGTCACCAGC
GTCCAGGCCGCCGGCCCCTTCCCCGCTGCCCAAACAGTAGAAAAGCAGGCGCCAAGTTGTTTTTG
TTAAAAAGGGGACACACCTCGGCCGCGAAACTGCAAACCCGGTGTCAGACAGCTGTAAACCCGT
GTCGACAGGTTGTCAGACAGCTGCGGGGGCTGGTCGGGAAGGAGCCCACGGCCTCCGGGCCCAC
ACCCCGCCGCCCCGACGCGCGCGCCCACCGCGAGAGTAGCTGGCCGGGCCGGCACGGGGCACCA
CGTGCTCGCGGGAGGGGCGGGAGCGGCCGGCGAGGGCGGGCGGGAGGCAGGGAGGGGGCGGGA
GGGGAGCCAGGGGCGGGGCCTGCGCTCAAGGGGATGCCAATCAAAGCATCAACTTCAAATTGTG
TCTGAAAGCCCCGCCGCCGAGCGGAGGGCGGCCGCCGCAGTCGGCGCGCGATTGCGGATCCGGG
CGCAGCCGGGAGCCGGGCGCCTGCGAGCACCGGGCAGAGGAGCCGCGACCGGCCTCCATCTCCC
GGCCCGCCCGAGCGCGCCCGGCCGGCCGCCCGCTCCTCCCTAGACCCCTCGCGGCGCCCCCTGCA
ACCCCCTCCGGCCGGCCTCCGCCTCCCTCCCCGCGCCTTTAATACTCGCCCGCTGCGGCGGTCGCC
GAGTCCGCGGACATGTCCTTCCCGCAGCTGGGCTACCCGCAGTACCTGAGCGCCGCGGGGCCGGG
CGCCTACGGCGGCGAGCGCCCGGGGGTGCTGGCCGCGGCCGCTGCGGCGGCTGCCGCCGCCTCG
TCGGGCCGACCGGGGGCCGCGGAGCTGGGCGGCGGGGCAGGCGCGGCTGCAGTCACCTCGGTGC
TGGGCATGTACGCGGCGGCGGGGCCGTACGCGGGCGCGCCCAACTACAGCGCCTTCCTGCCCTAC
GCCGCGGATCTCAGCCTCTTCTCGCAGATGGTGAGTGCGCCCGGCCTCCCCCGCTTCTCCTCTGTC
TCACCCGCGCCAGGGCAAGGGTGGCGGGTCGCCCGGGAGGGAGAGACTACGGGTGGACCTGGTC
CGGAAGAGGAACTAGAAAGGTCCGGGGGCAGGTTCCCGGTGGCCGAGGCCGCGGCCCCCGGGG
ACGCAAGAGGGCTGGGAGGCCGGGCGGGTGACGGCTGGGCCATCTCGGCCTGGGAAAGCGGAA
GGCCCGGGCCAGGGAGCGGGTAGCGAGTGAATTCAGAGAGGCCGCAGAAGCAGGCCCGTGGAG
CGGTGCCCGCGCTGGAGGTCGGGGGCAAACTCGCCTGGCTCGGCCAGGGCGCCCGGGCAGGCCC
ACGGGGTTCCTGCAGGTCGGCCCGGCGTAGCGTAGCAGGACTTCCCTTCCTGGCCGCGGGTTCCA
CTCGCGCGGCCTCTTTAGTTTTCGAACCGAGTCTGGAAAACTTGGTTTTCTCCCTCTTTAGCAGCT
CCGAGATAGTTGTATCCGAGTTTGCCAGACAGACCCCTTCTAAGCCTGGTAGAGTCAATCAAAAT
AATCTTAACAATAGAGGTCCAAAGGGATGGAGAGGTCTCTCCACGGCGTGAGTGCGAATTTGAG
ATTAAACAAAAATTAAGTTGCAGTAATGTGCTGGTGTCTGAAACGGTGTTTGATTTTACTTTTGTA
AGTTGCCCAAGTTTTCATTTCATTTGCACAGAAAGAAAAGCACTTTTCTTCCTGCGTTACATAATG
GAGGATTAAAGAAAACAGTGTCCCTTGGCTTAAAACAAATGGTGTCCTCTTAGTCTCCCGTCCCA
GTGGGCGTTAGATGTCGGGGCAGGCGGCTGCACACTTAATTCTCCGCGGGGGCATTGGCCTGTCT
GCCGGTCCAAATCATCCATTTTCCTTGGTCTGACTGCAAGGTCGGTGCTTAAACTTCGGACGGCTG
GTGAATTGTGCGGCGGGCGCGGGGCCCTGGGAGGCAGCCCCCTCCTGGGTCGCTGCCCGCGGGA
TAAAGCAATTTCCAAGCACCCGCGATATCTCCCCGCTCCCCGCAGGAGAAGCGGGGAGTAAACG
CCCCTCAAGTGTGCACAAGCAAAGAGCGGGTTTCCCTGTAACTTTTCTTGTAGTTTTGAAAGAAA
GCGGCCCGGCTGCCTTTCAGGTCTCTTACTATCGAAAAAGATCAGCCCCCATTTTGTTCAGGCGGC
GGGGAGGCCGGGACGCGATGAGAGATTTACAAGGTGTCCTTTCAAAAAGAATTCCCAGTGGAGA
CGAGGCTGAAACGTCTTCTTTACAATTACAACCAAAATAATTAGAAAAGCGCAAAGTACATTTTG
GAACGATTGGGCAAAAACGAAATCTAGCCGCAGAAATGTTTTCTCTGCGGCCTCAGTCACCAAAC
TAATTAGTCCAAGAAATCTTCTGGTCTTTACAACTTTCTCAGAGTCCGGAACTCCCTTTGCTAACA
TTGCAACTAGACCATTTTTTCAGAGGATGAATATTTTTTACAGAAATTGCGAATGCAGTTGTGTGC
CATTTGGGAACCCTGCCTGTGTTTGCGGGGGAGGGAGAGAGCTTCAGTGTGAGGACCTGCACCCT
TTGTGGAGAGCTGGGGAAGGGAGATGTTTGCTGTTCTGAGTTGTTTTTCCCACCTAGAGGGATAA
TATGTAAAAATTATTCCCACCCAAAAGGTGTGTGTTTCTCCAGCTCTCCCACTGGTTCTGAGAGAG
TAAACTCAAACCCAAACCCTGATTCTAGGCCTAGGTTTCCAAGCCATTATAATTGGGTGTTTGGA
AGTCAAAAGATAAAATTGTATTTGAATGTCTGTCTGCGCAATTTATGGTAATAATGAGGCCTAAT
GAGGTTGTTAGAAAGATAAAATGTTATTTACCAAAAAACCTGATGGGATAATTTGACTTGCTGTG
TTTTACTACTGATTATAAAAAGAATATCGATTGCAAATAAATCAGCGCCTCTAAATGCCTGCAAA
CAGCTAGTGTTTGCTCCCTCCAGATCAAAGTCAAACTTAAGAGATGAAGTAACTGAGAAGAGGCC
TAGGATACTGAACCGGTTCCCCTCCTGGCCGCCGGTGGCTCCCAGCCCTTGCGTTAATATTTTACA
GGCTAAGCCTTCCTTTTGTATTAAAAAAAAAAATGGTGTTTTTGTTATTGTTGTCGATGATGGCCG
GGATTAAAATTTTAAATTACCTGTCACCTCTAAAGACCTTTTAATGTGGGTAAACCATTATATGCA
GATTAATTTGGAAGGCAAAGGACTGTGCTTTCGTTTTAAATTGCTGGCGGATTTAGACCGGTAGA
AAACCCGGGATGGTTTATTTTGATTGAGCCCCCTCTGGGTGGCAGAGAGGAGGCTTGGGCTCTGG
GCCCTTTACGTTTGGAGAAATGGCTTTATCAGCTCAGTTGAAAGGTTTTTCCCTCTAGCTAGTGAA
AGATAAACTTGGAAATGCAGGTTTCTCCAGCGGTTGGTGGTGGGGACAGGGGTCGCCTAGGGAA
CTTGCAGGGGCCGCGGCCTCTGTTGTGCTCTTCTGGAGAGTGCACTGTTTGTGGAACTTTTCTAGA
GTGGCAAAAACGATCTCCACTGTCGGTGAAAGGGCAGTTCCTGAAGTCAGCTCATGGTCCTGGCT
CCCCTTCTCCCCAGCAGTGAACTGGGGGTGACTTCCTGATCTGCCCAGCACAGGAGAGCCCCGCA
AAGCGCCTGGGAGGCCCTCGAGTCCATTGAAGCGGCTGCTTCCCACTCTCCCGTCTTGGGGACTC
ATGTCTCTCTCTCTCTCTCCCTTTCTCTCTCCACTTCCCTCCTCTCTCTCCTCGATGGATCTGCCCTG
TGGCTTCAGGGCTCGCAGTATGAACTGAAGGACAACCCTGGGGTGCACCCCGCCACCTTCGCAGC
CCACACGGCGCCGGCTTATTACCCCTACGGCCAGTTCCAATACGGGGACCCCGGGCGGCCCAAGA
ACGCCACCCGCGAGAGCACCAGCACGCTCAAGGCCTGGCTCAACGAGCACCGCAAGAATCCCTA
CCCCACCAAGGGCGAGAAGATCATGCTGGCCATCATCACCAAGATGACCCTCACGCAGGTCTCCA
CCTGGTTCGCCAACGCGCGCCGGCGCCTCAAGAAGGAGAACAAGGTGACATGGGGAGCGCGCAG
CAAGGACCAGGAAGATGGAGCGCTCTTCGGCAGCGACACCGAGGGCGACCCGGAGAAGGCCGA
GGACGACGAGGAGATCGACCTGGAAAGCATCGACATTGACAAGATCGACGAGCACGATGGCGAC
CAGAGCAACGAGGATGACGAGGACAAGGCCGAGGCTCCGCACGCGCCCGCAGCCCCTTCTGCTC
TTGCCCGGGACCAAGGCTCGCCGCTGGCAGCAGCCGACGTTCTCAAGCCCCAGGACTCGCCCTTG
GGCCTGGCAAAGGAGGCCCCAGAGCCGGGCAGCACGCGCCTGCTGAGCCCCGGCGCTGCAGCGG
GCGGCCTGCAGGGTGCGCCGCACGGCAAGCCCAAGATCTGGTCGCTGGCGGAGACAGCCACGAG
CCCCGACGGTGCGCCCAAGGCTTCGCCACCACCACCCGCGGGCCACCCCGGCGCGCACGGGCCCT
CCGCCGGGGCGCCGCTGCAACACCCCGCCTTCCTGCCTAGCCACGGACTGTACACCTGCCACATC
GGCAAGTTCTCCAACTGGACCAACAGCGCATTCCTCGCACAGGGCTCCCTGCTCAACATGCGCTC
CTTCCTGGGCGTTGGCGCTCCCCACGCCGCGCCCCATGGCCCTCACCTTCCTGCACCTCCACCACC
GCAGCCGCCGGTCGCTATTGCCCCGGGGGCACTCAATGGAGACAAGGCCTCGGTCCGCAGCAGC
CCCACGCTCCCAGGTACAGCTCCAGGCCGCGTCCACCTGTCCCCTAGCTGGGAATGCAGAGGCCT
GGCTAGGTGTGGTAGCGTGGGGTGCAGCATGAGCCGGGAGGGTACCAGGCAGTGGCCGCTGAGC
CCTGGGGCTGCGCTTAATCCCTGCTTCAATTTAGAAAGCCAGACAAGGCCCTAGGGCTCTCCCAA
GAGAGCTTTGCCCTACCGGCGGGCCTGCTACGGGGTGGTGGTGGGGTGAGGGGTGACGTTTTTCG
GCGAATCTGCCTGGGCAGCCGGCAGAAGTTGGTGGGAAGGAGGCCTGGGACCTCTCCCGCCCGT
CTCTCCGTCCTAACTCTGCCTCTTCCGATCTCTCGCAGAGAGAGACCTCGTCCCCAGGCCAGATTC
GCCGGCACAGCAGTTAAAGTCGCCCTTCCAGCCGGTACGCGACAAGTGAGTGCTGTTTGCTTTTG
CTATGGGAGAAGGCGGTGGGGAGGGGGGAGGAGGAGTGGTCGGGACCCGGGCGGAGCTGGCTG
GGTGGCGGTGGGGGTCGCGCAGTCCTAGTTGAAGGAGCGCTCCCCGCCAGCCCTGGGCGCCGGG
CGAGCCGAGGAGACTGGAGTTTCTCCCCAGCCGGGAGCCGCGCTGGCTGTCGACCCCGCCCCCAG
GGCTCCGCTACTGGAACCGGCGTCGCCCGGCGCTGCGTCCCCCACTCACAGTGCCCCTGTCTTCTT
GTCTCGCTGTGTTTCCCATGCAGCTCTCTGGCCCCGCAGGAGGGAACGCCGCGGATCCTAGCAGC
CCTCCCGTCCGCCTGATTAAGGGTCTTCTTTTACTTTTGCGGGGGGGAGGGGGGAGGAGTTGGGG
AGGGAGGGAATGTGGGAGGAATTAAGACAAATATTTCAGACTGGTGTAAAGGACAAATATGACA
ACGACGTCAAGGACTCGCATCCGTCGCTTTCTGCAGAAAGGGGCTTCTTCGGTCCCGAGCTCGCG
TCCAGGTGGCCAGGCCTCTGCCGGCGGCTCCAGTGGCTGCGATTATCGGGTTCGGTAAATGCCCC
CACGTGCTTGTGTCTCTTTCCCCCCTTTTCTGTATATAGAGTGGTTTCAGATTGTAAATAGCGCGTC
AGCGAACTTGTCTAAATCATATATTTTTGTCTAATAAACTAAATGAAATGACACCCCCTCCCCGCT
CCTGCTGCTGTGTGCCTGTCCAGCGTGTGTGTGAGTGTGTGTTTGTGTGTGAATGTGTGTGTGTGA
GTGTCTGTGTGGCAGAAACAGAGACAGAGAGAGAGAAGTGGGGGATACAGGGATCCTGGAACC
CTGGGTGGGACCCAAGGGTCTGTGGCTGGGGGAGATGGGCTTCTCAATGGGGGCCTTTAGAGACT
GTTGCCACCCAAGACGCAGGTGCTTTAAACATCTCTTCGTTGTTTGTGGTTGTTGTTGAATTTTTA
AATATTGTCACTGTGGCAGTTTCTTGCTGGCAGTTCAATTGCTTTCACGAACATTTTTCTGAGACA
TAATTTTCTCAGGACATAAATAAGTTCAATTTGAGGCAGTTTTACAAAACGATTTTATAACGTCGG
TAAAAACAGAGGAAAAAGAATTTTTATTGCGACCCCAGAGGAGAACTTCGGATTAGAAACCAGT
TTACAACTAGTTGTCTCAACGGCGCATCGTGGCGCCTGGTCGTTTTCTGAGTTGAGTGTGAAAATA
ATGGAGTATCGCTTTGCATGTATTTTTAGTGATTCGGTTAAATCAAACACGGGAAGAAATTGGAA
GGCTCTTTAAAACTCCACAGATGGGCCAGCCGGGATGCGGTGCGGGGCTTCTCTGCGGTGTGAGG
TGTGAACGAGGGGCTGAGGCTGTGGTGGGAAGCGAGAAAGAGGAGGTGGCTTTGGTCTCCCAGG
GAAGCCCCTTTACACTTGGGCTCCACGGACTGCGTCCTTTGCCCTCAGGCGCGCGCACCGCGGGA
GTCCAGAGCAAATTGCCCTTAGATGGCCGCGGCCGGGCAGCGGGGAGGCAGCTGGGAGCAGCGA
TGTTGGGAAACACTCGCAGCGGGGCTGGCCTCGGGCGCGCGCGAGTGGGGAAAGGCCTAGGAGC
CTGGACATCGCTGCGGATCCGGGACATCAGCATCAGTGGGTTCGGAGCGGGACGCGCGCCACGC
GCCGCAGCAGGCACCTTCAGGAGGCTTTGCGGACCCGGCGCGGGGCCTTCAGGGCGCAGGCGAC
TCAGCGTTGAATGCGTGAAAACTGAGCCAGCAAACATTTCCAAAACTGCCAGCGAGGATGTGGG
CTGCCGGGAAAAACGGTCTAGTGGGGACAGGGCCGAGTCCCGAAGTCAGAGCCGAGTCCCGAGG
TCAGAGCGGCCGTCCTCCGCTCGCACCCCCAGCCTGTGACCCGCCCTTCCCGGCTTGCTCGAGAC
CCACTGGCGCCAGTGCTGCGCGTGGGGACTCCGTGCATGGCCGAAGCGAGGGGGAAAGTCGGGG
CGCTGGTGTCTTTTCAGAGGTTCCAGGAAAGAGGGAGGCTCGCGTTAGGACTAGGAGGTGCCAGT
CCACGGCTCCTACCCGCTCCCGACGCCCGCATCCTTCTACAGCCCTCCACCCCGTTCCTGGTCCCT
GTAGAGGGGAAGGTCCTCTCCCTGCCCCGAGGCGGGAGGAAAAGCGGCGAAGAGGAGGCTCGA
AGGGCGCCGCGTAGGGCAAGTGGGCCGAGGACACCG
CGI: 140 range-chr5: 5192442-5193941
CGTGTACTCGCGCACACGCAGTCAGGTGTGGATGTACACCCGCGCACACCCAGGCACATGTACAC
CCGCGCGCTCACACACCCCATCCAGCTACAGCAGAATTCTGGCCAGGCTGTTGACCGCACACCTG
CTGCCTCCTTGGCCACCCTGTCCACACAGTAGCCCGATCGACCCCCGTGGCGGCCGAGACCCAGG
CCCATCCGCAGCCCTGAGACCTCCCTAGGGATTGCACCCAGCAGCCAGTCACCGGCCTCCGCGGC
CTGGCCAGTTGAGGGTGGCCGTGACCGCGGGGCCAGGAGCGCCGCCACATCTCGGGGCAAATGG
CGCGGGGGAAGAGTTTCCTCCTCAGCCTCCCCGTCTCCGATCGCTCCGCAAACTCCAGAGCGAGG
CACGCGCCTTTAAAGGCAGGTCCGCGGCTCTCCCACGTCCTGGCGCCCGGTTTTCCGCACCCAGT
GTCCCCACAGCTGTGCCCGGGCACAGAGGCGCGGCCAGACCGCACTCCGCGGGCTGCAGGTGTC
CCGGCCTCTGGCGGCGCCGGTGCGGCCCGGAGGTGGGAGCCCGCGGAGCCACTGCAGTAGCTGG
AGTCCCGCCGAGTCCCCAGCCCCAAGGCAGGGCAGGAGCGCGCACCGGCCGGAGGTCCATGCTG
AGCATCGCCCGCGCCGGTGCCCGGCAGCCTCTCCAACTGTGTGGTCCCCGCGCGGGCAGAGAGGC
ACGGACTGCAGGCCGTGGGCAGCTCCATCTTCCCGCGTCCTCCTCCTCTGGCGCTGCCCGCTGTCT
CCCGCCTTCCCTCTGCTCCCCGCTCGCCTCCGCCTCAGCGCCCCGCTGACCTCGCCTCCTCCCCTCT
GCTCTTTGTCCCTGCACTCTCCCCTCCTCGGTCCTCTGACCCCCCCGCCCTCACCTCCTCCCCTCCT
CTCTCCCCTGCCCGCCCCGCGCTCTCCCACCGCTCCCGCCGCCCCCGCCGCCGCGGCTGCCACTCC
GCCCCCCGCGCCGCACGGAGCTTCAGTAATAACCCCGGCGCGGCGGCGGAGTCGCTGTGGGGAA
TCCTCCCGCGCTCTGCCTGGGTCGGGTCCTCCCTGCCCGCTCGCACGCTGCCGGCCGGGGACCCTC
CGGTGGCCCCTAGCCCCTCGGAGCGCTCCTGGATGAAGCCCCGCGCGCGCGGATGGCGGGGCTTG
GCGGCGCTGTGGATGCTGTTGGCGCAGGTGGCCGAGCAGGTGAGTCCCGGGCGCTCCCACCAGC
GCGGAAACCGCGGGTCCGGACAGCTGGAGGCGAGTCCCCCGCGGCTCCTCTCCCGCGGACCCCG
CCGTCTCACCGCGATGTCGCCGCTGTTTTCCGCAGGCACCTGCGTGCGCCATGGGACCCGCAGCG
GCAGCGCCTGGGAGCCCGAGCGTCCCGCGTCCTCCTCCACCCGCGGAGCGGCCGGGCTGGATGG
AAAAGGGCGGTAAGTCCGTGAGGTGGGGGCTTCTAATCCTTGCCATTTAGCAGCTCGTAATCTCC
GTGCCG
CGI: 21 range = chr5: 6790705-6791004
CGGATCGAAACTGTGGTGAAAGACCTTTGGCCGACGGCTGATGTGAGTATGTTCTTTGGAGTTCT
GTGTCGCACGTCACGTGCGAGTAAATTTAAATACCCTGTGATGATGATGTGTCGGCTAGATCCAC
ACAGACCTTTTCTCGTTGGCCCGAGGCAGCAGTTCTCCAAGTGTGCTTTGAGAAGGCCTCCGTTGC
CTGGAATGCATGGCCCCCGGCGCACCTGCACCTGCTGTCTTAGACACCTGCGGTGGCCGCACATC
TTCGTGATGCTGGCGCGCACTCAAGTGCGAAGACCATCG
CGI: 54 range-chr5: 54554812-54555385
CGGGCTCGGCGGCGGTCTGCTCGCACTTACGTCGCCAGCCCAGTCTCGTACCCGAAAATTCAAGC
CCCATCCGAGACAGGGAACCCAGCAGGCTTGCACTGCCACGGTGGGGAGCGGGACGCACGGAGC
ACGACACTGACTGGGGGAAGGGGGCAGCAGTTCGCGGCTCCTGCAGAGCAGCTGCGTGGCGGGA
ATGGGTCCTTCCACCGGCGGTGCGGCGGCCCTGCGCCGGCTCCGGGCAGCCGAGTAGCCCGCCAC
CCACCAACTAGCTAAGCAGCCGCCTCTGTGAAGCTCGGCGGTTCCCTGTGCGCCTGCGAAATTTT
GACTCCGACTCACCAGCGACCGGCCACCGAGCCGCCGCTGTAGGAGCTGAGAGCACGTCTTGAA
CACCGGATCTTTCCACCCAAGACCCGACAGCGTGCAGGGGCCTCGAGCAGTAATTTGAGGCCGCG
TTTCCCGCCAAGGTTTGGCCCCAGCTAACCGCCCCACCCATGCAACCGAGCGGGAAGAAAGCTGT
GATTCGAGGGGCCAGGAGAATACGGGAAAAGCTTCTGTTCTGCGCACAGCCAGTGCG
CGI: 129 range-chr5: 114542616-114544119
CGCGCACAGGGAGTCGAGTGCTCCTTTGTGCCATCACCTCTTCTCGAAGCTCCAGGCGCAGAGGG
CAAACGCTGACGCCCTTTCCCGCCTTCTCTCCGTCTGCGAGTTCCGGAGGGCAGTGCACTGTGCA
GCCTCCGTACTTCCCGACACGTTCCGCCTGCAAACCACTTAGGAGCTTCTGCCGGCAATTTCTCGG
ATGCAGCGCGGAGCCCAGCCTCCCGCACTGACTCAGGCCCGGCACCACCCCGCCTGCTGAGGGC
GTCGTCGCGGGCGCCACCGACGGCGCCAGCCTGCGTTCCTAAAGGCTCCAGGACAGGTGTGGGG
CTGAAATCCCGCTCCTCCAGCATCTTCCCCTCCCTACCAAAAGAAGGCTGCTTTAAATATTCCCGG
CCTGGAGCAGCAGCAGCTAAGGACCTGGAAAGAGGCCCGAAAAGGCCAGGGCAGGGCGAACGA
ACGTGCTGTCCCTATTTACCGGCCACACGCTCTGACTGCGGGCTGACCCTGATCGCACACTTCGA
GCCTGTTCACTCGCTGGGCGGACCCTCCCTCCACGCTCCGCGCCCAGGCAGGCGAGGTCACCCAC
TCGAAGTCACCACGTTTGAGTAGCCCTGGTTGGTGGGGCAGCGCCCCCTTGGCCAACGCCCCGCC
CTCCTCTCCATCTTCCGACGCTGGGCTCTCGCCCTTCGGCGTCCGAGAGCCCCTATTGCTGCCCAG
CTGGCCCCAAAAGCGTCCGAAAGTCCCCTCTTGGCTTATTTCAGAGATGCCACGCAATAAAGAAA
AGGCGGCTACTTTGCATTCCGCAGCTGCTGGGACGCCCGGGCTGCGAGCGCGGCTCCTGGATTCC
AGCCTCCCGCCCTTCCCAGGCGCTGGAATGGACACGGACGCCCACAGTGGCGGGCCAGGTAGTG
CCGGAGTCGGGGGCCCAGGCCGCGGCGCCCCGCGCCTCATCACTTACCTTGCCTTTAGCTATCAA
TTCCATGATGTAGCCAAATTCACTCATCTCCCCAGACTCCGACATGTTTACACCCCTTCACAAACT
CTGGAGGACCGACGCGGGTGTATCGAATTTGTCCTTTCTTTTCTCTTTTTCTGTTTTTAGTCTGAGT
TTTGCCGAGCTCCCCGCCCATAAGCTGTTAACCAGGAAAAGAGGGGAAGCGCCGGGGAAAGCAA
GAAGCGGGCTTGGGTGAAATGAAGGCCATCGAGGGCTCCCGGGCAGCCTCGCCCGGCTTGCTAG
GATCCGGGTGCAGCGGTCGGGGCTGCGCGATCTAACGGAACAAACGAGGGCGGCGGCGGCGGCA
GCAGAGGCCGTGAAACCCTCACCAGGCGGAGCCGGGCGCGCGGAGGCCCCGCCTCCTCGGGGGT
CGCGGCAGCGGTTCGGACCGCAGCGGAGACAAACAAACGCGCACACAAAGCGGGTCCGCACTCG
GCTCAGCGCGGCCCCAGCTAGACATTCCCCGGCCTTTATCGCATGCCGGCAGGTTGGCCGTCCCC
GGCTCTCGGCGCG
CGI: 21 range = chr5: 127899858-127900162
CGCGTCCCGATTATCAGTGCAATGTGATCACTTGATTTTAAACGCAACTATGCGTCCAAATGGCT
GAGTGCAAGAGGTCGGCCGGAGATAGGGCCGAGTGCGCTGCCCCAAGCTGCGATCCCTGCCAAG
CACTCACGGACAATGCACTGGTTTCCTCCAGGGAGCGTCTTCCATCCAGGGCAGCAGTAGGAGTG
GAATCTGGAGCCGCACACGTTGGGCCTGTGATGGACAAGCGCGGTCACGTAACAGATAGGTAGA
GGGATGCAGCAACATATAAACGGTCTTCGTAAGCAATGCCCCGAGCG
CGI: 56 range-chr5: 135720297-135720941
CGCCAGTCCTTTGTAGGCGTTCATGCGCGAGCGCGAGTGGCTGAAGGAGTCTTTCCGCTGTTTCTC
GGTGCACTCATTGCACTTGCAGAAGTAGTCGTGGGGCCGCTCGATGCGGGCGCCCTTGAGCAGCA
GGATGTGCACGATCTCATACTCCTGGCAGTGCGCCGCCAGGATGATGGGCGTGATGTCGTGGGAG
AAGCGCGTGCCGTCCTCGTCGTAGGCATAGAAGTCGTCGTCGCGCAGCTCCTGTTCCAGCGGGCT
GAGCGTCAGGCGCTGGCCCTGCGCGAAGGCCGGGTGGTTGAGGATGGCCTCCACGATGCGCACA
TAGCCCTTGCTGATGGCCAGCAGCAGCGCGTCCCCCACCCGTGCCAGGTTCTCCTTCTTCAGCAGC
AGCTCCGTGACCTCTAGGTGCTCGTTGCCCACGGCCAGCTGCAGAGCGTTCTGCCCCATGTAGTC
CACACAGTTGAAGTTAAGGGTCTTGGACTCCTCCAGCATTTTCCGGACCACCGGGATGTTGCCAT
ACTCAGCCGAGTCCAGGAAGCGCTCCTCCTCGGGCGTCAGACTGGTGCCCTTCTCGTTGAACATG
TAGGCGGGACCCCGGATGGCCTGGCGACGGCCCTTCTCCCTCAGCGTTGTGTGCCGGCG
CGI: 29 range = chr5: 140780945-140781256
CGAGAAACCGCGGAATATCGGCTTAGAGCCTGCCATGGCGAATCGGCTACAGCGCGGGGACCGC
AGTCGGCTGCTGCTGCTGCTGTGCATTTTCCTGGGGACGCTGCGGGGGTTCCGGGCCAGGCAGAT
CCGATATTCGGTGCCAGAAGAGACCGAAAAGGGCTCCTTCGTGGGCAATATCTCCAAGGACCTG
GGGCTGGAGCCCCGGGAGCTGGCGAAGCGCGGAGTCCGCATCGTCTCCAGAGGGAAGACACAGC
TTTTCGCTGTGAATCCGCGAAGCGGCAGCTTGATCACGGCAGGCAGGATAGACCG
CGI: 182 range = chr5: 141235749-141238152
CGCGGCGTCTTCCTGCACGAAGCGACCCAAGCAGCCCCAGTCCGCACCGCCTTGGTCAGCGGGCG
CTCCCAGCACACACCCAGCGCGCGGAGCGACTCCTCCCAGAGGACGCCGAGTGTGTGTGTGTGTT
GCGGAGATTAGGGGGTGGCGGGGGGGACGGCGGCAGTTTCTTGGGCTCTGGCGGCCCCACCCCC
ACCCCGGCAGCGCCCAGCCTCCAGGAACCCAAGGCAAGGCTAGGGCGCGGCGTGGTGAGAACGC
GCTTCGGCTAGTGGGGGGAGCCCTTGCGCCCCCCGCACCCAGGCCACCGCGGGGAATCCCTCCTC
CCCGGCATCTCCGGCCTCGGCCTAGGGTCCCCCGCAAGCCGGAGCCGGCGCGAGCGGCGCAGCG
GGCGCCAGGAGGGGGCGGGGAGGGAGCCGGCGCCGGAATCACAAAGCCGCGAACCCGGCTAGG
CGGGGACATTCTCATAAAACCGACCCTCTCGTGGCGAGCAGAGGGGGTTTTTTGCCCTACGCTCA
ATCCTCCAAGGCCTCAGTGCCGCCCCCAGTCTTTCCTGGCAGAAGAAATAGTAGCCGACCACTGT
TGGGGAAACTGAGGCCAGAATTCTGACTTACAGAGGGTAGCTGGGGGGAAGGGGTGCGAGACCA
CCTCGCCTACAAATTCTGCTCCTATGCCTGCCGCGATCTCCGTTCTGGCTCCAGGGCCTGCCCGCG
CACACACTGGCCTACAGCGGGGACCAGGCGGAGGGGGCGCTCAGGTGTGAGACGAACCCAGGTG
TGCGGGACCCACCAAGGTGTGTGGGACCCAGGTGTGCTGGAGACCCAGGTGGCTCAGAAAACTC
AGCTAAATGGCTGGAAGAGGGTCGCAATCACCGTCCCAGGCTAGCACCCTCTACAAAGGAACCT
GGGGCCCCAGAGCTCCGGGGAGGCGGGATGGCTTGGCCAGCCAGGTCCAGCTTGTTCCCGGAAC
ACCAGCCGGTTCGGCCCGGGAGTGGGGGTTGGGGAGGCGCCGACTGGGAAGCCGGCAGGCCGGC
CCAGCCTTCCCGCTGTGCCGATATTTACAATAATAATGTCGCGATTAACGTGGCGGGGCGCTTGG
GGCTCGCCGAACGACCCCGGCGGCTTCTCCTGCCTGAATCTGGACTCCGAGAGAGAGGAACGGA
GTGTGTGTGTTTGTGTGTATGCGCTGCAGGGTGTAATGTAATGTGACACTGTTGGTTTAAGGTGTG
TGTGACACCCAGTGTTAAGATGGGTGAAGCAGTGGATTCTGTGTTTGTGACCGTGAGGCGCTGCT
GTAGGTGCCAATTCCTGTGTTAGAGTGTGTGCCATCAAGCGTGTGCTGAGATACTGGGTTGTGTG
TTTGTGTGGGTGCAGTAGAGGGGTGCAAGGAGACCTGTGCTGTGTACCTATGTTCTGGCCAAGAA
GTGGGTAAGATGATATATGCTAGGGTGTGTTCCTTAAAGGAATGTGGGGGGTGGCTAGGTTTTGA
GGGTTCATCTAGTGAACTGTGGCTGTGACACTCGTACATGAGGGCACGAGATTGTGTTTGTGTGT
CCACGTCGCGGGGTGTCACACCCGGTTGTGTTATTGCTCTGTAAAGCCACGGGTTGTGTGTCGGT
GCTGCTGATCGTGCAGACACTGAAGCGGATTCTGAGCTGGCGACGGTGCCCCAGTGGGCGCTGGT
GTGCGGGAGGTATGTCTGTGTGCACATATGCAGGGATGTATGTATGTGTGTATATATGTCCACGG
TCTGGCCCTCTGGGTGCCCGCGAGAAGCTGGAAGCGGCTGAGTGAGCCGAGGTAGTTGCCCAAC
AAGTGTGTATGTATGTGTTGTGGACAACGGTCCTTGCTTGCTCCGTCCTCCGGGAGACCCCCACCA
AACTGGGGGCCGCGCGCTTCCCCAAACCCATCCTCCACGCCGCGCGCTCCTGGAGTCTCCTTATC
CCCGTCTCTCCGAGCGCGCCCAGGTTTGGGACGCGGGGACTAGGGTTCTGCTCAGCACCCCCTCC
CCTCCCTCGGCTTCTCGCCGCGGACCTGGGAGGACGGAATCCCTCAGCCGCATCCCCTGCTATGG
GCTCCCAGCAGCCCCCACCTCAGCCCCCTCGCGCCGAGCTCGTGTTGGGCCCCCGCGGCCTCGCT
CCGCCGAGCGCCCCTCCCTCAGCTCCCGCCGGCCATGACCGCTTCGGGCCCCAAGCCGCTGCTGC
CTCCACCGCCGCCGGATCCTTACCCGCCTCCGGGCAGCGCCGGCCGCCCGCCCCGCTGTCCATGA
GCCGCCGCCGGCCCCGGCCTGGGCTGCGGCTCCGCACGGCTGGGGCTGGAGCTGCAGTTCGGGCT
CCGGCTCCGGCTCCGGCTGGCTCTGGGCGCAGCAGCCCGGCGGCTTTGCGTCCGCGCCGCGCTCC
CGCTCCCCG
CGI: 263 range = chr6: 1326743-1330210
CGCCGCGCAGAGTCGGCCGAATCCAGCTTCTCCAAGGGCGGGCGGGTGGGATGATTTGTTGATTT
GCTTGTTTTCCACCAAACCGCAGGGCACCTCCAGGAGCTCCGGGACAACTTACAAATGAAGACAC
AGAGCCCCCGTCCCTTGCTTCAGAGAACCAAATAACGGAACTGAATGATCGTTTCTTCAGCAAAG
CCTAAAGACGCTTGCGTCCCGCGGAGAGGTGCCCACGCGCGACTCGGCTGCCCCCTCTTCTTGCG
GGTCCGGGTAGGGTAGGGCGGGAACGCGGGACGTGCCCTAAGCGGGCCAGCCGAGCGCGAGAC
AGGCGATGCTGCAGAAGCCTGCCCGCCTGCGGGTCACACACCGGCCGGCCTCGGCTGCAGGGGT
TGCGGGGCCACGGGTCGCGGGGCGCCGGGAGCCGCGAGCAGGTGACGGCCGCGCCGGGCAGCCC
CGCGGGCCGCGATTCCCGAGAGCCTGGCGCCACCCCGCGGAAGCCGGAGGAACTGCGGCTTCTT
CCCGCTCCCGCGCGGAGGGGAGAGGGAGGCGCAGCGCGGAGGGGAGAGGGAGGCGCAGCGCGG
AGGGGAGAGGGAGGCGCAGCGCGGAGGGGAGAGGGAGGCGCAGCCCGCACCGCGCAATCCCGG
GGCTCTGGCGGGAGGGAAGGTGACTTGCGTGGGACGACTCCAAGAGGCCAGGCCCGGCCTTCGC
AGTTCACCCACGAGGATACCGCGACCAAGCCACCAAAGCACTCTCTGGCCAGGCCGAGACCCCTT
TTCCCCCGGGCCCCAAGAGAAAAAAGCACTTTCTCCTAAAACACGAACTCCCCGCGGAGGGGCC
CGGGGCTGCCTGGCACGGGGCAGGGTGTGCGGCTGCCGTGCGGGCGCGGGCCAGAGTCACGTGG
AGGCAGGCGGAGAGGGCGAGGGGGCCCCCGTAAATCATCCTGAGGGATTTTTGGCCCAAGTCCC
TGTTTCCGAAAGGTTGCGGGACGGCTTTGACGTTGATTATGTAGCTGTTCTCTTTTCAAGTGCGCG
CCCCTCCCCACCGCAGCCCGTCTGGTGTGCGAGGATTGTGTTTGAAAGGGAGGAAGCATCCTTGA
AGTGGTGGGTGATGGATTGTCTAAACCCTTCTGGGACGTTCTGGGTGGGGGTTTTCGAGGCAGCT
CATCAATAAAGACCCCCCTGGAGAGAAGGGGCGGCTGGAGACCAGCCCCCGCCGCGCAGCCCCG
AGGCCCCAGGCCTGGAACACAGTCCTCGGTGTGGGGCACAGGGCGTGAGGGGAGGATGAGCTCC
CCCTGAGCAGATGGAGATTTCTCCTTGCCTGGGCCGTTTGACCCCCAAAGCCTTGCAGACTGGCC
GCGTGGGTGCCATGGGGCGATTTCTTGGGAAAGGAGGGCAGCAGAAAGGCCCGTGGGCTCTCAG
CGCCTTGGAGTCAGGGGAGCAGGAAGGGGAGTGGAGGTGCGGACACGCCTCCAGCCACCGTGCT
CCCCTAGGAGGATCTCCCTTCAGCCCCTGTAGAACCCTGCATCTCAACCACCCTCGTGCATGTGGT
TTACCTTGGCATAGATTTCCAAGCCGCTACAGAAAACCTGTCCACCTCCCCCAAGGTGGAGGCGG
GAGTTGGGAGGCAGGGGTGCTGGAAAGGGTCACCTGGCTCAAGCCGACCCCAGGTCTGACGGGC
GGAGAGCCTCGAGGGCCACGCCCCAGGCCAGTGGTGTCCTCTGAGGCCGCAAAGCCGACTGGCT
TCTGGAGGAGCAGCAGCCTCGGCCTCAGCCTCGGCTGGGAAAAGACGCAGTCGGGGCCCTCCGT
GCTCCAAACTTGAACTACTCTGGGTCCCCACGAGGAGGAGGCTGCCCTGGGGTCAGAAACTCCGA
CGGAAATGAAGGGGAGGGGGCGTAGGTTCCCTGGCACAGCTCGGCCTGGAGAGGCAGCCTGGGG
GCCCAAACTCATCTAGGGCGCGCCCGGAGCTTCCCCGGCCTCTCTTGGCCCATCGCGAACGAGCT
CCCCTCCCCGACTGGGTCGGCCTCGGGCGCCTTTCGCCTTCGCAGGCCGCCGGGGTTCCGCGGAG
GGCTTGTGTCCCTCCCTCCCGCCGCCGCAGCCCGCCCGAAGGTCAGGCCGCATCCGGGGCCCGGG
GCCTTTGGCCAGGCTGCGGGGAGGGGTGGGGTTGGGGGGCTCCCCAAAGCAGCTTCTCGGAGGC
GGCGTCGGGGCGCCCTGAGGTCCCTAGTCGGCCTCTGGGCGCCGAGCACGTTTTATTTATCCCCA
CAGAGGATGCTGGAGGACAAAAGAAATATTTGGACGTCAGAGCTGATTTATATTTTTGGAAGAG
CTACGTTTCCTTTTCTGAAGAGAAGCGAGCGCGCCCCATATCCTGAGCTCGGCGGGGAGGACGAG
AGGGGAGTTTTGATGCGCCAGGAAGTTCATCTATCGCCCAGGCGCTCAGGACCCGGCGCCCCGCC
GAGTTCGCCGCCCTCGGAAATATTTATTTATTTGGGGGAAGGGAGGGCAGCGGGGAGGAGACCA
GCCAGACGGGAGAAGAAAGCCCTGCCCGGGCTGGGTTGGGACCTCCAGGGCGCTTCCCCGCGCG
GCCGCAGCCGGCCAGCCTCTGGTCTCGGTTGGAGGGCATCGCAGGCGCGGCCTGGCGACCTCCGG
GCGCCTTTGGCTGGTGTCTTCCGTAGCCCTCGGCGCGCACCCTCCCGCGTGCAGTGCCCGGAGTG
ACCTCTTCAGGCCCCGCGGGCGCCTCCTGGGTGGGCCCCGCGGCCCCTCCAACCCTGCCCAGCGT
GTGTGCGCGGCCTAGCCATCTTTCTCCCCAGCCTGGGAAGGCGGGCGGGAGGAGGGGATCCGAG
GCCGGGGGCCGCGGAGCAAAAGGCACGCGGACCCCTAAACGACCCCCGAGCGTTGCCACAGCCG
GGAGGGCGGAGGGAAGGTGTGCAGAGCTCGGGGGTCTCCCAGGACGCAGCTTGGCTCCTACCGG
TTTCCAAGGCATCTGCAAGCTCTGTCCTGCGACCTAAAGCATGGGGACGTCCCTTCCAGCAGCAT
CAGTGGGCGAAAAGTTAACATGCTGAGGGCCACCGGCACCCACACTTGCTTCCAGGAATACTTGC
GGTACAGGCTTCCTGACTTATTTTCTTTAGCCGCTGGGTGCCCCCCCTTCTTCCCCCGTTGGGAGA
CGCAGACCGACTCTGTTCCAACCTAAGCCCCTTCTCTCTGCCTTTTGGACAAAGCGGGCCGCCTCT
GCTCTCCACTCACTCCCCGCAACCCCGCAACTCGCCACCCCTAAACGCAGCGGGCTGTAGGCGAA
GTCGAAGAGGTGCTCCGCCTGGTACCGCGGCCTGCGCACTGGGATCGGGGCCTGCAGGCCGCTG
GGCTGGCTCCTGGGATCATCCCAAGGTTTTGGTCTCGCAACCGAGGGGACCCGCGCCCGGCAATT
TGGGAAGTGCGGAAATGGGGTGAGGCCGGTCTTCGCTTGAGGGTCTCG
CGI: 190 range = chr6: 5947471-5950124
CGATGCGGATTCGCGCTGGCGCTGAACGCTGAGCGCAGGCGGGGAGGCTCGGCACTCTCCGACC
TCAGTAGCGCCCCCAAAACCGAAGTCCAGCCGGCCAGAGAGGACACTTACGTGCACACGGTCTT
GATGTTCGTCTTGTCGTCCAGGCCCTGCGGGTAGTTGGCCATGCTGTCGCCCAGCTTGAGCAAAC
AGTCCGAAAAGCCCTTGAAGACCGCATCGCACTTGCCCGCTGCTCTCACGGCCTGCACCAGATAC
GCTGCGGGGAGGAGGGAACACCGGTCAGCAGCGCCACGACCCCGCCCTGCCGCCCACTGCCCTT
TCCTGCGAGACCCTGGCTCCGCGCGGCCTCATCGCATCGGGCGGCCTCTCCCAGCGCCCAGCCTC
GGGGCTCGCCAGTGTTAAAGGTGAATGAAAGACGTGACCCAGGAATGCCCGACCTGCAGCTAAC
GCTGCGTCTAGTGCAAATTGTCGGTGTGTGAGCAGGTGTGGGTGGGTGGGAGGTAGGTGAGTCCA
TTCCTTTCCTCCAGCTCCCTCCCGCATCTCGCTCCGTATCTTTTTCTGTTTCCATCGCCCCCTCTTCT
CACCTCCCTCATCCCTTTTATTTCTCTTTCTCCTCGCTCCCTCCTTCGTCTCCCAACACTTCCCCAGC
AAGCCCTGTCACAGCTCTCGCAATGCGATCTTCCATTTTGAGGTGACAGAAGGGGTCTCGGCTGC
AGCGTGCGGGGGCGAGAACGGGGTGGGGAGACGACAGGGCGTGGGGTCACGCAGCTGAACCCA
ACGCCACATCACGAGAATAAGCCTCAGGCTTGGCGCTCTGTCCTTGGTGGGCGCTCAGCAAACAC
TGCTGAATGAATGAACGAATATAGTCCCTGGAAGCCACCAGCAGTTACCGAAATGGATTGTTTCA
TTATAAAACCGATTGCCTACCGTGGACTCTGTATTCCGAGTGAGACCCTCGGATGCACCCTCTCCC
GCCCCGCTGGAAAGAGGGCAGCCTGGACGTCCTGAGACCTGGCGCGGATGATGAGTGGTCTGGA
GAGGAGAAGGCCAGAGGCCGGGCGGGGTCACGGATGAGTCTGCCTGGGTCACCTCCGCAAAGGC
CAAATCCACGAACCTCGGGCCTCTTTCTCTCCTCCTCCCCTGTCCGCCACTCGGCTCCCCAGACAC
CCTTAACAACCCACCCGCCCGCCAAAAAGCCCGCCTACACTCCGGACGGCAGCGGGGAGAGGCT
GTTCTTCTCCAGGTGCCAGCCCTGCTGCTGAAAGTGAGCTCGCCGCGGCCCGGGGCTCCTGGCCC
CAGCTCAGGACTCCCTGCAGAGAGCAGGCAGGGATAAAAGCTGAGCGGCGGGAGGAGGAGGAG
GAGGAGGAAACACAGGCCGCACGAAGGTCCAAGCCCCAAGCCCCCAAGCCCCCGGCCTCTGGAC
GGCCAAACCCCGAGGCGCGGGACTGGAAGGACAGGTACCAGGCTGCGGGCGCGCGGCTGTGGCC
ATCTCTTTCCGCCCTGAGGCCGACGAACCCGGCTGGAAGCTGAGTGCCTAGCGGCCCAAAGCAGC
CCGGGCGCCGGGAGGGCGCCAGAGAAGCACAGCGTTAGGGCGGGGAAGAAAGGGTGAATCTCA
GAATCGAAATCCGCACTGGCGCCCACGACCCTGGGCGCCGGCCTGGTCCTCGGCAGCTTTCTGGC
GGCTGCGCTTGTGTGTGAATGTGTCCCGGGAGGACCGGACACCTCAATCCCCCGGCCCCCAACGC
GGGCGCCTGTCCGCGAGCGCCGGGCCAGACGCCGAAGAGGAAGGTGACCGAACCCGTAGCAGCT
TCCGAGAGCGTACCCGTTTGCAAATTGCTGCAGGAAGAGCGAGGCGGGCCTTGCGCTTTTTAATC
CGGAACGGGAAGCACTGGGGAAGGGACCGAGGTTAACTTCGACCTCCGCTGGGGCAGATACGTA
AACCTTCTCAAACCTCCGAGTTCACCTTACAGCGAACATTAAATACCCTTGCGACTACAATACTA
ACAAATCGAACACTGACGTAAAATCTTAAGAATAAACGAATCTTTGTTCTGATAGAGCTCGTTCA
AAATGGAAATACACTCCACACATCTTCCTCTATAACGCATGTCACAAATACAGCTTTGCAGAGAA
GAAACGAAATAAAATAAAGTCCAATCTCCCCGTCCACGTCTCTCCCCACCCCTTGCCTACACCCC
CAAAACTCAGTGGCGAAAGAAGCCCCTAAAATGCATTGACATTTTTTAAGGGGAATTGGGGCTCG
CCGGGGGGGGAGCGCTCTTGTCACGTTATCTTCCCAGGCGCTGCTGGAGAAGACCCTGCACACC
GCGCATGTGTGAGTGTGTGCTGTGTAAAATAAGTCGTGAACATTTCTGTCTTTTAAAACTGTGACT
CCTTGGACTCTAGCCTTCTCTGGGAGGCCGGGAGGCTGCGCGCGGGACGGATTTTGCAGGCGCAC
GCGTCCCAGCAGGTCGCAGCCCGTGGGTCCCTCGCTGTCCTTCCTGAACCCCGCTTAGGCGAGAC
TCTAGCGGGTGACCCTGGCTCCTTCCCTGTCCTCTCTGCCGTCCCCTTCTTGTGCGGTTTCCATCCG
TTCATTCATTCGTTCGACCTCTCATTCATTCCGAACTTCCTCCTGCTGCGTCTTCTTTCG
CGI: 40 range-chr6: 10498025-10498551
CGTTTCGGGTGCGGGGTGCTGATGCTGCTTTTTTGTTGTTCGTTTGCGCTCGCGCTCGCTCTCGCTC
TCTCTCTGCATCCCCCTCACCCCCTTTCTCGGAGACTGAACTAAGTGAAAAGTTGTTTCAATAATC
GCAGCTCTCTGCTCCGCCAGGGCCGAGGGAGGCGGGCGGAACACGGAGGGTGTTTTGTTAAATG
CTCCCGTCGTTCGCAGGGGCTGGGACTTGATAAAAGGAGACAGTTTTCTGAAAAGATTTGATTGA
AATGGCGTGTGCCAGGGCTGATGGGAGCCAGCGAGGGACAAAGCGCCGAGAATCCATGGACACT
CGAGCAATTATGCCTCCACGCTGAAGGTGGATTAGCGCGCTGGAAAGAAGCATATGTTTGGCCCG
GGGCGACACTTCCCCCCGGCTGAGCTTAGAGAATGGGAGCGCGGAGAGCGGCTGGACCCGGAAT
ATCAACTATCTGCGAAGCCCCCCCTTCTAGCCCAACTCCGCCAGCCTCCCCGCCCCCGCCGGGGA
AAAGTCG
CGI: 100 range = chr6: 22677564-22678684
CGTCCGAGGGTGGGTCCAACGGGCTTCGAGTGTTCGGGAGATAATGCACACTTCGCAGTGGATCT
TCTCGGGGCCCCGAGCGTTGCCCGCCCATTAGGTGGCGCCGGTCCCCTCGAACAGGTCGCTCAAC
AACTCCCGCCCAGCAGCCGCCCTTACTGCGCGCGCGCAGACTTCGGCGTCTACTTCCGGTGTGGC
CCAGGCGGGGTCCGCAGAACCAGCTATGTCGGCCTACGGCATGCCCATGTACAAGAGCGGGGAC
CTGGTGTTTGCCAAGTTAAAGGGCTATGCCCACTGGCCGGCGAGGATAGAGCACATGACCCAGCC
CAACCGCTACCAGGTGTTTTTCTTCGGGACCCACGAGACGGCCTTCCTGAGTCCCAAACGCCTGTT
CCCGTACAAGGAGTGCAAGGAGAAGTTCGGCAAGCCCAACAAGAGGCGCGGCTTCAGCGCGGGG
CTGTGGGAAATCGAGAACAACCCCACGGTCCAGGCCTCCGACTGCCCATTAGCCTCAGAGAAGG
GCAGCGGAGACGGGCCTTGGCCGGAGCCCGAGGCCGCAGAGGGCGACGAGGACAAGCCGACCC
ACGCTGGTGGCGGCGGCGACGAATTGGGGAAGCCGGACGACGACAAGCCCACTGAGGAGGAGA
AGGGGCCGCTGAAGAGGAGCGCGGGGGACCCGCCGGAGGACGCCCCCAAACGACCCAAGGAGG
CAGCCCCCGACCAAGAGGAGGAGGCGGAGGCGGAGAGGGCGGCGGAAGCGGAGAGGGCGGCG
GCGGCGGCGGCGGCGACGGCCGTCGACGAGGAGAGTCCGTTCCTCGTGGCGGTGGAGAACGGCA
GCGCCCCTAGCGAGCCGGGCCTGGTCTGCGAGCCGCCTCAGCCAGAGGAGGAGGAGCTCCGGGA
GGAAGAAGTCGCGGACGAGGAGGCCTCCCAGGAGTGGCATGCCGAGGCACCGGGCGGCGGAGA
TCGCGACAGCCTGTAGTTACCAGCGTTTCCAGAAGAGCCCCTGCCCCGTTCCTGCTGCGGCCTGG
CCGTTCTTGGGGAATCTGACCACGGCGTGCAAACTGGGACTGCCTTTCCCTCTCCTCAGCCCGTCC
TCCTCCAACCCGCGCTCCTTTGCCCTGCCG
CGI: 20 range-chr6: 25834434-25834701
CGCCGCCCAAAAGCTTATTGAGTTCCTCATCATTGCGGATCGCTAGCTGCAGGTGGCGGGGAATA
ATGCGAGTTTTTTTGTTATCGCGAGACGCATTGCCTGCCAGCTCAAGGATTTCTGCTGTGAGATAC
TCTAACACTGCCGCCAAATACACTGGTGCGCCTGCCCCTATCCGCTCTGCATAGTTTCCCTTACGA
AGCAGACGATGGATCCGGCCTACGGGAAACTGCAAACCCGCTCTAGAAGAGCGAGACTTAGACT
TGGCGCG
CGI: 71 range-chr6: 41176454-41177326
CGGCTTTATCGTAGGAGCGTCTTCGCCTCCCCCGTTTCCAGTAGAAAAGACAGCTTTGCTCCTTTG
AAAGCGCAGACCGCCGCACCTCCAGCCCCTTCTCCCCGGGGAAGTAGGCCCCGCTAAGAATGTG
GGAAGGTGGTGGGGCGGCGACTGAAGTCGCTTCCGATTGGCGTTGTCCCAAGGAAGCCTGCGCG
GATTGATCGGCGGCAGGCCTCCAATAGAGCCTGCTAGGCGGATTGGCTGCTACGCGGCTGGGCCC
TGTTTCCGGTACCTAGGCGGGCAGCCATGGTGACCGGCGAGCGGCATGCGACGCCGCCTCTGTGG
CCTGTGGAGGCCCGCTTGGCGGCGCTGCTTCCCGACCTACTGGTCTTTCGGAAGCCTCGGGGATG
GGAACCCGAGCTCGCCACGGCCCAGGGCGTCCTCCTAGGCGTCCATGTGACGGGTGAGGGCGAC
GGCCGGCACTTGCACTTAAGTCTCCTGGCCTGCGGGAGAGGCGGCCGTTGGGTCTAAGCCCCTGG
AGGCCTCGACGGTTACAGGCCTAGCCTCCCGCGAGCTAAGGCACAGTTTAACTTCGTGCTGCGGC
TTCCTTAGGAAACTTTGAGTATCTTCGTTTTAGCGTGGGAGGACATCTTTTGGGATGAACCGGCTC
AGTCCTTGAGCTCACCCCAAAGATGATGAAATTGAAACTCAGAGGAAAGGATTTTTACAGGACC
ACGCCCCGGGCTTTGAACCCGTTTTTCCCAACCCCGAGCATTTTCTATTAAGCGTTCTGTTTCAAT
TATTTTATTCGTTCAGTTAATTCGTTCTTGGCGTCATAAGTATATGCCCGCCGTTGTTCTGGCCGTA
GCTATCTAGCAGCCACTCCGCGAGTCG
CGI: 68 range-chr6: 50895246-50896050
CGTCCGAACAGAAATCGCAGCCGCCTCCTGTGTGGGCATCTTTCACGGAAATTTCTGCCCACGTT
CAATGCGTACGGGTCTCTAGCACTGAGGCTAGCTCAACTCTTCGGGGCAGGAGAAGTTGCGCCCA
GCCAGGAGGACGCCAGCCGCCTCCGGGGCCCGGACCGCGCTCGCCAAAGCCCTTTTCCGGCTCGG
GCGAGGGCTGACGCGGCCAGACGGCTCCGCTACTCGCGGGCGAGGCGGACTAGGCGGACGAGGC
GGCCGAGGCGGGCGAGGTGGGAGAGGCGGGCGAGGTGGGAGAGGCGGCCGAGGCGGGCGAGGC
GCGGGGCGCGCGGCGGGCCAGGCTGGGGACCGGGAAGCCCGCGGAGCCTCGCTTTTAAGCGGAA
TGGTCTGGGACAGCCCCAGGTGGTTGCCAGGGGAAGGGAGGAAGGGAAGGTTCTGCTGAGACGT
CGAGGTTGGAAATAAAAACTTCGGAAAGGCGAAGCGGATTTAGAAAAGGAGGGAAGTCGAGGC
GGGCGAAGCGGGCCATGGAGATTGCTAAGAGGAAAAGTTGGAGAGAAAAGGAAGGAAGAGAAG
CAGACTCCCGGGTGGCGGGCGCGGGCCGCTAGGTCAGACGAAAGGTGGAGGGATGCAGAGGCTC
CCCAAAGAGCCGAGAAAGACACGCGACCCCTCCGGATCGGGACAGCTCCTGAAAGCCCGGCGCA
GAGCCGCCTCGAAGATCCTAAGAGTGGGCGACTCACAGGCGCGGCCGGCAAGCTCCTGGGGGAC
TCGGGCTCGGACGAGCGCCCACAGGCAGCGCCTGGCG
CGI: 43 range = chr6: 75968300-75968853
CGGCGCCGTTTTGGAAACCCATCTCGGAGCGCCCCACTGCCTCCGCGGGAAGGTCCGACGCACCC
GGCTGTCCCTGAGGGAGGCAGGATGGAGCAAGGGCAGGACACTGGAGGTGGTGCCCAGCAACTC
CGGCGCGCGCTTGCTTCCCAGGCTGGAAACCTTGGCCCAGGCTCAACCTGTCTGAGCTGGACCAG
TCCAGGCCACGCGCCTTGGAGCCCTGCTAACCAACAGGGACAGCCGCGACCCTAGGTCGTGCGA
ACACCTGGGGTCCCGGATCTCCGCTTGCCTGCCATCATCCCCCAGGCTGCTTGGCTGCTGGCTCAT
CTGCCCGCACCGTCCGCCCCGCCGACTGAGCTCCCCACAAAGCACACAGAGGCGCAGAGCCAGC
GCTGTTGGAGTCTTTTATTGTGTTCTGTCACCGAGGTCGCAGCCCCGAGCCAGGTGGTTGGTTACC
AGCCAAGCGAGCGAGTGCGCCCGCTCTTCGGGGCTCCTGGGTGCCACCCGGAGGTGTCCTGTTGG
TTGCGCTGATTAGAGCCGTCGCCCCCTCTCGGGCG
CGI: 27 range = chr6: 101953488-101953856
CGCCTTTTGTGCCGGGCTGTGGCTCGCTATCGACATCTCGTCCGTTACCAAGGCTGGGTTTCCTAC
TGATTTCCCTCCTCCTCTGCTTTCACAGGCTCGCGCGGCCGGACATTGTGGGTGTGCGTGCTGGAT
TTCTCCCGGATGCTCTCCGACTAACATGGATGTCCCACCATTCCTTGCAGTGGAAGGTTGTTCCTT
GGCGCAGTGAGTGAAGAACATGCAGCGATTGCTAATGGGTTTGGGAAGCGGAGACTCCTTCCTCT
CTCTATGACCATGCCGTGATCGTGTCTGCGGTCACCACTCGACGCATCCTCATTTCTACCCGAACC
CAGGAGCCGAACGCTAGATCGGGGAAGTGGGTGCCGTGCG
CGI: 318 range = chr6: 108592365-108597232
CGCCGAGTTCGAGAAAGCGCTACGCCGCCGGTCGGGCTAGCTCCACAAGCGGCTGTACAAGTTG
GCTGTCAAAAAACGCTGATTTCTCCTCCTGTCACCTAATAAACCCCTACGCGCTTATGGCCTCGTC
CCACAATCCCCCAATCTCGTCCCAATTCGAAAAACCGAGGAGGAGGGAATAAACTGAGAGATAA
AGATCCCCCCATCTTGCTCTTTCCCCGGGACCCCAGCCTTGGTCGCGGCGCCCCACTAAGGAGGA
CACAGGCTCTGGTGTGTGTGGTGTGCGAGACCCCGAGCTCGAGGCCGAGCCAAGGCTGGGCAGA
AAGTTGCAATCACGTGCTGTCGGAGCCCACTGGAGCGCACAGCCCGCTCCCCCTGGGACGCCCAG
GCGGAGGACCTGCTGCGCCCTCCCAGGGCTCGGGGGACTCCAGCATTCACTTGCACGCACAGGCG
AACTCTGATTGAAAGCCCGGGATGACACCGAGTCTGGAGAAAGAGGGACCGGGGGGTGGGCTGG
CGGAATTGCAGAGCGCCGGCCACAGCTCCCCTCCCCGCGAACGTCGAGCGGAGGGCGGGAGGTG
TAACCTCTGACCTCTGGCCGGGTCCACGCCCTGAGGAGGGACTGGCAAGCTCTTGTTCGACAAGT
TCAAGCTGCCGAGAGAGCTTAAATAGAATTAATCTCTTAGAGATCGGGGATCATCGCTCCCTCGG
CATGCGCTCTCCCAGCGCCGCGCACAGAGCAAGGCGCGAGAGAGCTCAGGAATCGCGGGAAGGC
AAGCGGAATGGGGAGGGGGTAGGGGATGAGGGCCTCTCTTCACTATTCCTCCGCCCGGAGAGCG
GGAGCCCGCAACGCCCGCCGAGGACGAGCGGCGGGAGGGAACGCTCTGCCCTCCAGCCGCCCCG
GTGCAGATAATGGAGGCGACAAGAGATTCGCTCAGCGTCGGATGGGCCAGCTCTGCTTGGGGAA
GCTGGCGGCATCCTCCCCTCGGCTGGTGCCCAAACCCACTGCGCGAAGGCCGAAGGAACGCGGA
ACCTCCAGAAGACCCCATCCTCAGCCCTGACTTTCCGTAGATATGTGCAAAATGAGTAAATTACT
CACCTCGGGCCAGATCCAAGTTTTACCCAACAGAAGGGGCACCGGACCAAGAATGAACCAACTC
ACATGGCCATGTCCGGCGCGCACAATCACACGCCAGCACACAGCCACCCAATTTCTTCCGCGAAT
CTATCTGGCACTCTGGAGAGAGGGGGAAAAGCGTTTTGAGAAAGCCCCGTCACCCCTCCCCTTCC
TTCTTGCCGTGAAATATACGAATTCATTTTTATTACGAGCCGCACCGTCCTCACCATCACGCACGC
ACAGAGCCACACTCCCATATTCACACTTTCTAACTCGTAAGCTCCGACAGCGCCTGCATTTTCTTT
GGGAGCCGCTTGGAGGTTCATTAATATCATTAGCATTTAACCCCCTCCCTCTTCCCATCCCCTCCC
CGCACATGGCTGACGTCAGACCCCGCCAGGAGTTGGGGGAAAAGCTAAGTGGGCCAGGGACGCC
CTATTCCCCTCCCCGCGGCTGCCTGTCAGAGCGCTTCTGGAGATATTACAGGGGACCCAGCCCGC
AGCGACAGGCACAAAGTCACGGGGTAATGAACTTCGGGGACCCTTCGCCGCTGCGTGCGCGGCT
CTCCCCGGAAACCCGGACCTGGCCGCCTCTTCCCTCGGAAGATTTCCCAGCAATCTAGTTTTCCCA
CTCTGCGCTTGGGTTCCGGCAGCGCGGAGCCCGTCTGCCTCTGAGACTGCGGTAGTGTTTTCCTTC
TTTCCTTGGGAGACCAGCGGTCGGCAGAGATTGCCCACACTCTGCATGCCTATGTAGAGGGAGAG
ATCGAAGACTGAGTGACAGGAATGGGGAAAAAGAGGGATTTCGCTCCGTAGGAAGGCCATTTTC
GTGTCTCCATCTCTGTCTTTCAACATCCCTCTCTTGCTGTTCTTCCTTCTTCCTCAGTCTTCCTGTCC
ATCTCTCCATCTGTCTGTCCATGTGTGTGTCCATATCAAGCAGCATTCCCAGCAGCTGCGGTTTTG
CAAGAGCCGGGAAGAAACTTAAGGATGCTTAAATTTCCACTGTTGGACGAATTCTGAGCGCCCAG
GGAGCAGCGCAGCGCGCGACTGACACCCACCTGTCCCGCCCAGGAGCCTTGCAGGCTGGAGGGC
AGCTGGAGAGCGGCGGCGCCCGGCGGCGAGGCGGGCGCTGCCGGCCGGGACTCGGGCAGCGCCC
ACCAACCGCTCCGCCCCGGGACAGCCAGCATGAGCAAGCCAGCCGGATCAACAAGTGGGTACCT
CTCGGGCCGCCGTGGGGCCTAGGCGCGCAGCCTGGGGCGAGCGAGCGGGGAGGCTGGGGGAGGT
CCTGCCTGGAGCGCTGCGAATCTGAGCCCCTGAGAGGGATTCCAGCGGGCGTGTGCGTTCGGCCC
AGACCTGTAGACCGTGAGTTGGAGCATTTCGTGGAGAGGGGAGAGCCGTTTCGTTGCCTCTGGAT
TGCTTGATCCCCCCTGTCTGGTGCGGTGAGAAGGTTACGACCCGCGCAGCCCACCAGTCGGATGA
GTTGTCTCCATTTAGCCGCCAGGTGCTGGATGGGGGGGCCATGGGGGCGGGAACTGGGCCGCAG
CTCCAGGCGGTAGCACAATAACACACTCGCTCAAAACTCCGAGCTCCAGCGCGCAAAAGCAACT
CTGTGCAAAGCGGATTTTGAATGGAATGCTTTGCACCCCGTTTCTAGCTATTTCAAATAATCCTGC
AAACTGGGAAGCAGAAACAATTTAAAAGTCACATTTTCCTTAATCCTAAATCCGCGTAGGTCATA
ACTGGGGAATTTAAAGTATGGCGAACCACTCTAGCAAAGAGAGGACCAAATCCCTAATCCCAAG
GACTTTTCGAGCCGGAGCCCAGCAGAGGCAGGAGTGCGCGGCCTGCTCCCTCCGTGCGCTTCTCT
CCTTCCTCGAACTTCCTTAGCTGCCGGCTCTCCGAACGCCAGGCCGCAGCTGACCTCTCACCACCC
CGAGACTCACGAGCGCAGGGCTAAGTGTGTGTGCGAGGGCATTTGCTTGCACCCTGCCTGCGGAA
CCCAAGAATGTGCAGGCCCGAGCCAGCGTTGAGCAGGCGCGGTCACGGTGCTCAGATCTCCCGG
GGGCATTTCAGTTCCCGCCATCCAGTGGCCCACGGCTGCGGGCTCCAGGGTCTGAGGCTGGGGAC
TACCGTTGCCGCCGCAGTCCCCATATCCCGAAGTTGCCTTGCTGCTTGTGTTGTTTTCGCAGATAG
CATTTTTGGCGCTCTGTGCGTTCCTTCCCTCCCCCTCCCCCTTTCACTCGCCCTCATTGTCCTGAGT
CTTTGAAAGTTGGGAGAATCGGAGATACTTCTGAGGACTGGTAATGAAGTCTCACTTAAGTGGGA
TGCAATTCCCGCCCTCCTACCCCCCTCCAAGAAGGAGGTTGTGTTTTCATTTTGTTTTGCTTTGGGT
GCTGACCTTTAAAAAATTAGAGCAAAATGAACGTGAACAAAAAGAAAAGGAGAAATGTTTCGAG
CTGGGGCAGAGGGAGCAGAGAAGGAGCCCTCACCGCGGCCGGAATGCAGAGCGGACCCTGGCC
CAGGACTGGGTTTCCCTTTAGGCTCGGGCCTACCCTGGCCCTCGCTGTTGGAATCTCCAGGAGGT
AAAGCGACCTCGATTTTTGTTGCCCGCATTCCCGGGCGTGAGTGTCCTTCCCAGGAGGCTCAGGA
GGCCGTTTCTGTTGCATTCTGAGCCTCCGTTGCAAAAACTGAAGCCCGTGGGTCTCGGCAGGCCT
CCTAGCTCGCTCGCCCCGGGACAGGCCCTCGCCTACACCCCTGGAAGTAAGGAGCCCCGGGCTCT
TTCGTCCTTTTCGGGGTGTGGAGCCCCTGGGGCCCTTGAAAGGTGAGGCCTCAGAGGCGAGGGAG
GGGTGAGCGGGGAGCTCTGCCCGCCTGCGGCTGCGCCCCCGCTGTGGACTAGGAGGCAGGCCAA
CCCTCCGGACTTTGGGGGAAAAACCACAGCGGGCTCCTTGCGGAAACTTTGGCCGTTCTAACTTG
CCAAGAGCCTGAGTGAGGCCTTGGAAGCCTCCAGCCCCGGCTCAGGTCGGGACGCGGCTGCTGA
GCTTTCTCAGGCCCGCAGGACAGCGGCCCCCGCCGGTGGCGCCGCTGCATTTAGGCCCTTTCCAG
ACCGGTGGCGGCAGCCAACCCGAGACTTGCGTCCCTCGGGCCCGGGGCAGCTAGGAGGTCGGCG
CGCAGCGGGCCGGGTCAGGACTGGGTCGAGCAGACAGAGCTGCAGCCCCCGCCTTGCCCGGCTT
CTCGCGGCTGGAGAGCAGAGCGATGTCACCCGGAGCCCCGCCTGGGTGGTAACGAGACCCTGGC
CAGTCACCCCTGCAGCCCAGACTAACTTCTTTCAACAGCCTCTGATGGTAATTACAGTAATCGAA
GCTGCCATATATCTTTAGGCAATTATGACACACAAAAAGCCCCGAGGGGACCCCCTGGCGAGGG
AAGTTAAGAACGGTTTTCCAGCTTCAGGAAACTCCGGCTCGCCTCACGTCGGAGCTCGCTCGGCT
TGCTAAATGAGAGGAGCTTTGCAACGGGGTCAACCAGCTTGTCTCGTGACCCCAAGTCACCTTAA
CGTGGCTGGGTGGCGGAGTCTGAGGCACAGGCCCGCTATGCCCCGGAATTTTCGCGTCCCTCCCT
CCTGGGCCCCGCCCCAGCCCGGTTGCCTGTTTCTAATCTGCCCCGGGAGCCGCGGCTCAGAGGTC
TGCTCAGAGGCAGGACTCGCACTGGTGGTGGCCTAGAGGGCAACAGTCCGGAAGCTCGGGCGGG
GGAATCCG
CGI: 38 range = chr6: 126122028-126122524
CGCGGTACCTGAGCTCCGTGGAAGGCCTGGACTCCTCGGATCCGCTGCGGGTGCGGCTTGTGTCT
CATCTCAGCACTTGCGCCACCCAGCGGGAGGCGGCGGCCATGACATCCTCCATGGCCCACCACCA
TCATCCGCTCCACCCGCATCACTGGGCCGCCGCCTTCCACCACCTGCCCGCAGCCCTGCTCCAGCC
CAACGGCCTCCATGCCTCAGAGTCAACCCCTTGTCGCCTCTCCACAACTTCAGAAGTGCCTCCTGC
CCACGGCTCTGCTCTCCTCACGGCCACGTTTGCCCATGCGGATTCAGCCCTCCGAATGCCATCCAC
GGGCAGCGTCGCCCCCTGCGTGCCACCTCTCTCCACCTCTCTCTTGTCCCTCTCTGCCACCGTCCA
CGCCGCAGCCGCAGCAGCCACCGCGGCTGCACACAGCTTCCCTCTGTCCTTCGCGGGGGCATTCC
CCATGCTTCCCCCAAACGCAGCAGCAGCAGTGGCCGCG
CGI: 270 range-chr6: 166499964-166503413
CGTCGCCTCCCGTTCAAGCAGCGTCCCTTCCCACAACCCCCGTGCAGAAGGCGCAGCGCGGCCCC
CTCCTCGCTGGTCCCAGACCTGGCGGGCTCCTCACACCTACCTGGCCCCCTCCGTTGAGCTTGTTG
GTGAGCTTGACTTTGCTGAAGGAGACGGGAGCCTTCATCCAGTGGGCCCCGAAGTTGGGCGAGTC
GGGGTGGATGTAGACGCAGCTGGGCGCCTGCGGCTCCGGCTTGCCCCCCGGCACCCATTCCCCGT
TCACGTACTTCCAGCGGTGGTTGTCCGCCGCCACGAAGTCCAGCAGGAAGGAGTACATGGCGTTG
GGGTCCAGGCCAGACACGTTCACCTTCAGCACCGGAAACATCCTCCTGGAAAACACGGGGGGGG
CGCAGGAGGACCCCGACACTGACCAGGTAGGCCGGAGGCAGAAGCTGGGCACAGAGGCCTCAGT
TATTTCGGGGCACAGAGGAGCCCCCTGGGGAACGTCCGAGGGTGACTCCCAAGCTCCCCCTTCCC
CGAGCCCTGCCAGGTCCAGGTGGCCTCCTCACCATCTCCACGGCCTCGGGAGGGAGGGGAGGAG
AGGACCTGGCGAAGGGTTTCAGTGCAGGAGGCCACATCCCGCGGGGACAGGGGGGACCAGGGC
GCGCCTCGCGGGTCCCGGGATGCCTCCGAGGTCGGGACACCGAAGTGCGCGTTCCTCCTCCCCAA
GCTTCCGCGGAGAAAGCCCCAGAAGAGGGGCTTGTAGAAATGCACTCGAGGGAGTTAACCAGAG
CGGGAACAAACACAAAGCCCTCCTCCAGGAGAAAAAGGGTTCGACCTCCGGGAACTTGCCAGGT
CCCCCAGGCTGCCCAGGCGCTGGAGAGCGCGGCGCGCGCGGGCTCCGGACGCGCACCCACCTGC
CGTTCTTGGTCACGATCATCTCATTGGTGAGCTCCTTGAAGCGCAGCCACAGCTCGCTCTCCTCCA
GGCCCACGCGCAGTTCGCGCTCTGTGGGGTCGCCCTTCTCGCTGCCCGCCTGCAGCTCATTCTCCA
CGGCGCTCAGCAGGTGGTCCACTCGGTACTGCAGGCTCTTTCCCGCGCTCTCGGTGCCAGGGGAG
CTCATCCTCCCGTCCGGCTCCCCTCCCCGCCGTCCCCGAAGCCCAGACTCGCTACCTGAGATCCAC
CTTCCCTGCTCTTGGCCGCCGCCCTTCCGAGAAAAGGGGCCCCTTGGACCGAGACCTGCGACGGC
TCCCGGGTCCCGGGTCCCGGCACAGACCCGGGAGGAGGGCGCGGACCAAGACTTGGGGGGAGGG
GACGGGGGCAGAGGGGTGGGGAGAAGTTATTCCACTTGAACTCCCCAAGGCTCTACTAGTGTAG
GTCTCTGGGGACCGAAATTCAGTGCCCTCCCCATAAATAGAGCCGCGGCGGCAGCGCTGGGGTGC
TCGGCGGATTGGGCCGCGCACGCTTTGAAGTGCCGGGCAGCCTCCCATTGGCCGAGAGCGGCCAT
ATCAGACCCAGCCGGGCGGGTCTGGGAGGCCGGGGGCCAACAATGGGCTCCCGCACGCCTGTTT
CATGTAAATCCGGGGCCCCAGCCCTGCGAGCCCCGGCCCAGCCCCTACACCCCCGCCCTTCTCCA
AATGTTTGCACCTCCATCAAAGCGGCGGGGCAAGCCCCGGAGGAAGGTAAGCTGGACGCCCCGG
CCTGCCCCAGCCCGCCCCAGCCCGCCCCAGCCCGCACTTCCGCGAGGGGCTGCCCGGCAGCCTGC
GATGCTCCCTGATTCCCGCGCGGGGTCCTCCTCGCTTTCCCAGCGCGCCCGGATCCCGCAGCCGC
GCCAGGCTGGAACGTTCGCGCGGCCTCACCTGTCCCCGCTCCAGGCGCGCCTCCCTCCCCTCCCA
GGGACGAGCGCATTTGGCCGGGCTTTGGGATGCGCCGCGCTCGTCTCGCCGCAGTAGTAGTGCTG
TTCTCGCGCCTCCGATGCCTCAACTCTCCAGTCTGGCAGTTTCCGGTGCACCTGTCCCCACACGTC
CCTCGCCCACGGAGCCCCAGGCGGCGTTACGCACACCCAGGATCGTGGATCAGCCTGCCCCGGCG
TCGGGTGTCCCCGCGGCTCTCACCATCTGGAAAAGGAAGGTCCGCGCGCAGAGAGGGAAATGGA
CGGAAATAAGCAAAAGCAAAACAACCCCTTCTGAGAAGTGTCCTCCTCGCTCTCTTATAAAAACA
GGACTTGTTGCCGAGGTCAGCGCGCGCATCGAGTGTGCCAGGCGTGTGCGTGGTTTCTGCTGTGT
CATTGCTTTCACGGAAGGTGGGAGAAGGTCGGGGCTGGCGGGGTGGTCCGGACTAGTGGGACTC
GGGGCGCTTTCCCCGCCCGGCCCAGCGCCAAGCGGTGTCGGCAGCGGTTGTTTTATTACCTTTCG
GTGAGAACTTACGCGAGGAGAGCGAAGGAGAGGTGACAATGAGCAGGAAATAGTTCAGTGGGG
TCTCTTTAAACAGTAACATTCCTCCTAAAACGGGAGCCCAGGAAGGGGGAAGGAGGCAGAAGCT
CCCCCTTCCAAGGCCTGGCTTTGCGCTTCTGCACACCTCGCCCATTCGCTGGACGAGGGCCTGCCT
CGGGTAGCAGCCTCTGTTTCCCGGTCTCCTGAGCGGGCACCCCAGCCCCAACCCCTCTAGAGAAA
GCCTGCGCATCGGGTGGGGCAGCGAGTTCAGAGGAGTCTTGCGCCCTAGCAAGGGGGCTTTCCCG
CGGACATGGTATCTGCGCCCCGCGCTAAGTCTCCCCTGCCCCCTGCCCCCTGCCCCTCGCCCCTCG
CCCAGCCGGGCTCGCTCCGGGGTCGGGAATTGCTGGGGAAGCCGAAGGAGGGATCCCCGCCCGG
GACTCGAGGGATCGACCCGCCTCGCGGTATGCGGAGGGCTGCCAGGAGCCAGTCCCGGGGAAGG
GTGCCTGCCGCCCGTGGGGGACAGGGCTCCTTCTCTCCCTTCTCCAACTTACACCGACGAGTCAG
GAAGCCGGAGCGTCTAGTCCCTGTCCGCATTTCTCAGTGTCTTTTGATGTCTGGGATGCATGGATA
TTCCCAGGGGGCTGACCCGAGGTGGGGAAGTTTCCTTCTAGAAAGAGGCACCAAAAGGACCTCA
TTTGCTCCTGACGCCGGACCCTCCTGACACGCCTTCACTGGCTCATCGGGTCCTGGGCTTTGGGGG
AGAGCGTGGTGCGTGTTACTCTCCTTACTCCGGGTATTAAAATGTCTAAGAGGTCATCGCTTTGGC
CCAAGGCCTCCCGAGGATAAGATAAAAGGGAAAAGTGGAGGGGAGGTGAAGGAGGAACGAGGG
TGGGTCCCAGGCAATTGAGGGCCTCGGAACCCTAGGCACGGGAGCGAAACCTCTCCTGCGGCTTC
AGGGCAAAGGAGTCCCGAAGGCTGGATCCCTCCCCTGCGCTCGGACGGCGTCGCGGCCGCTTTTC
CCACAGTCGGGAAGCG
CGI: 25 range = chr6: 169801163-169801511
CGCGGCCGGGTTGATCTCCAACACGGCAATCTTCCCGCCAAAGAGGGAGGCCAGCAAGCACAGC
ACCTGCAGGGACGCGGTTTCAACATCGTTAGCGACACCACCCAGTAAAAGCAGACGAAACGTGC
ATCCGCACACGTATCCTAACAGCTGCTTCATTCTCATAGCAGCGACATCGCCCTGTCACTCCTAAA
ACGTTTTTACACACAAAATCCCGTTTACTCAGCCAGTCGTTACAGGATCACTCTGAAGAAAGAAG
AAATCAGAGGCACGTCTCATAGACAGGGAGGGCCGCGTCTCAGGACTGACCTTTTGATCCGCAGT
GCGGTTCCTCAGGGCACACGATCCG
CGI: 40 range = chr7: 1029023-1029492
CGCATACGAGTCCAGGGGCTGCTACAAAGCGCGGCGACGTCTCCGCGGCGAGCTGGCCACTCCA
TGGTCCCGGCGCAGACACTGGGGCAGGCGCCGGCTCTGTCGTCGAGGCGCTCACAGGCAGGCAC
ACGTGAGCTCCTGGAGGACAGGGAGAGCGGCCGCCCCGCCCCTGCGGAGCACAGGACGCTTCCT
GCCACCCCTGCAGAGCATGGGACGCTTCCTGCCATGGTGCCGTGGCAATGGGTGGCACCTGCCTG
TGGCCCCTTCTCAGAAGGACGTTTTAAACGCGTGAGGTCTGATGCACAGCCACAGGGAGACACA
GACGAGCAGATGTGGGCATCCGAGTATTTACAAGGTCTGGTGGCTCCTGCAGCCGCGACACGGG
CTGAGCGCAAGTGATGTGTGAGGTGTCCCCAACAGATGGCACGGGGAGCGCCCACACCCGCCAC
CGCGGGGTCTGCGGAAGCTCG
CGI: 26 range-chr7: 19113449-19113833
CGCTTCTAAACCCAATACGCTTTTCTGGAGTCCTCGGAACAGCTGGCCGGGGCTTTTAGCACACG
GGACGGGACCTTCGCCTTTGCCTGTTTTTTTTTCGACCAGGAGAAGCAGGTGCGAGAGTCTGGGG
CTTAGGAGGTGGAAGGCGGGGAAAGAGAATCCACTAGGTCCTCGTGTAGAGAACAACAGTCGCT
CCTTAGATATTACTCCAGGACGGAAACCTGATTGCAAACCGCTGTTCCTTCGAAACTTGCAAAAC
CCGGAACAGAAAACTCCCGCCCAGCCAATTTTAGCTCTCGCTGAACTCTCCCGCCTGCGTTACGTT
TGCACAGCAACTCTTTGTAAATGTCCGAGTCCTCTCGGAGGAAAGATCGTTAGGGCGACG
CGI: 207 range = chr7: 27149139-27152087
CGAGAGCCGCGTCCCCGCGGTCGCGTGGATTTAGAAAAAGGCTGGCTTTACCATGACTTATGTGC
AGCTTGCGCATCCAGGGGTAGATCTGGGGTTGGGCGGGCGGCGCCGGGCTCGGCTCGCTCTGCGC
ACTCGCCTGCTCGCTGCTGGCAGGGGCGTCCTCCTCGGCTCCGGACGCCGTGCCAACCCCCTCTCT
GCTGCTGATGTGGGTGCTGCCGGCGTCGGCCGAGGCGCCGCTGGAGTTGCTTAGGGAGTTTTTCC
CGCCGTGGTGGCTGTCGCTGCCGGGCGAGGGGGCCACGGCGGAGCAGGGCAGCGGATCGGGCTG
AGGAGAGTGCGTGGACGTGGCCGGCTGGCTGTACCTGGGCTCGGCGGGCGCCGCGCTGGCGCTG
GCAGCGTAGCTGCGGGCGCGCTCTCCGGAGCCAAAGTGGCCGGAGCCCGAGCGGCCGACGCTGA
GATCCATGCCATTGTAGCCGTAGCCGTACCTGCCGGAGTGCATGCTCGCCGAGTCCCTGAATTGC
TCGCTCACGGAACTATGATCTCCATAATTATGCAACTGGTAGTCCGGGCCATTTGGATAGCGACC
GCAAAATGAGTTTACAAAATAAGAGCTCATTTGTTTTTTGATATGTGTGCTTGATTTGTGGCTCGC
GGTCGTTTGTGCGTCTATAGCACCCTTGCACAATTTATGATGAATTATGGAAATGACTGGGACAT
GTACTTGGTTCCCTCCTACGTAGGCACCCAAATATGGGGTACGACTTCGAATCACGTGCTTTTGTT
GTCCAGTCGTAAATCCTGCCTGATGACCTCTAGAGGTAAACTCGTGCACTAATAGGGGAGTTGGG
TGGAGGCGAGGGGGGTGGCGCGCGCGCCCCGGGCGCGTGCCCGCCGCCAGTTGCCGCCGTTCAG
CCGGACTCGAGCGCCACCCGCTGGAGGCAGGGCTCATCGCCCAGCTTCCGACCGGGGGCTGCAA
GGGCCGGGGTCGAATTGAGGTTACAGCCCATTATGGCAAAATTATTGCATTTCCCTCGCAGTTCC
ATTAGGATGTACCAATTGTTAGGCCGTCAGCTGCCGATCGCGCGCCCGGCGAGGATGCAGAGGAT
TGGGGGGAGGTGGTGACTTGCATTTTATTTACAACAACTTTATTTCCCCCGTTTTGCAGCCCCTCT
TATTTTTGTGTCGAGGTTGGGGTCGGTACTGACCGTCCTGCCAGCAGCTCTGAATTTTGAAAATAC
AGATATCACCTTCGGGGAAGGGGGAAAGCCATTTAGCCAATTGGAGAAATAAATCCTGCCCGCA
GCAGCAGCAGCTACAATTACGGCTCTGTTTTTGCGAGCGCATGAGGGACAGTGTCCCTGCCGCTC
TTAAATGACAGGCGTCTATTAAAGATAGCTTTTGTGTAGTGTTTCTCCAAGGCGAGGTCAAATTCC
ATACACTTTTATAACCGTAGTCGATTTTTCTTTCGTGTGAATATGGTTTTCGTGTCATTAGTTTGCG
ATTTGATTTGCTTACGTATCCAGCCTGGAAAATCTTCATCACAGGGTCCGGTTCCTCGAGCCAGCC
GGGCCCCAAGTCGGAGGGTTCTCCTTGAACCCAGCGAGTGGGCCCAGGCTCCCTGCAGCCACAG
AGGCTGCCTGGGGTCTGGGGATCCGTGGGGCGGGTTACTGGGGTCTTGCTTAGACCTCCAGGAGT
AAAATGAGGGCGATAATGGAAGCATTCCTTGGCAGTGCCTAGTATCTCTGTAGTTATTTTCCACG
GCTCCGAAAGACTCAAGTAAATCACAAATATAGCTGAGAGGCAAGTGGAGTCTCCCCGCTGGAG
GCCCGGCGTTGCAGGCGCCCCTGGCACGTCTGGAAGCCAGGACTCTGGCGGCTCCCATGGCCCTG
GGCCCCTCGTTGGGTCCTGAACGCTGCTGTGGCGGCGACGCGGGCGCTATCGGAGGCTGGGAGC
GGGAATCCGGAGCCGGGAGCCTACCCCGGGCTGTAATGTTCCACCCGCGCCCAGGTTAACTCGCC
TCGGCTGAGGCTGCTTCTCTTCCACTGACGGTTGCACACGCGGGACCGAGAGACTGGGCTCTGTT
GGGGCCCCCTTTGTTCCTCGAGCTTCCTTCCTGTTCTGGGAGGCGGCTTGGGAGGCCGCGACAAG
GCCGGGCTCCAGCTCTTAGACCCCCTCTTTCCACTGGCCAGAGATGATTTGATGATGCCCTTCGGG
ACTTACTGGCGAGGGACTTAGGCAGAGACGCCCAGACACGAAACGGGGCTCGGCCCAGGGCTCT
TTCCTCCCCAGCAGCCCCGCGTCCCGAGGTCGGGGAGCTCAGAGACACTAGCACAGGAGCCCCA
GACGCATTCAGGGCGCACCCCAGAACTCCGGAGCCGGTTTGGGCATCCTTGTGGAGCGGGACTG
GGTGTGTGCAGTGCGCCCCGCTCCACCGCTGGTATTGGCTGTGTGTGAGGTTTTGTTTTGTTTTGT
TTTGTTTTGTTTTGTTTTGTTTTGTTTTGTTTTGTAAGAAATAAATGCACAGACGCTTGCAAAGCTC
CGGGCTCCCCTGAAGCTGCGGAAGCCCCCAGATGGGAGCAGGCGGGGAGAAAAGTTGGGGAAC
AGGCGAGGGCAAGGGGGCAAAGCCGAAGGAGGTTGCAGCGCTGGCCTGGTCCCTGCCCAGGCAT
CTACTCGCCCGCCTTTGCCTCTGAGTCCTCCCCGCTGGGCTGCGTGGAATTGATGAGCTTGTTTTC
CTTTTTCCACTTCATGCGGCGGTTCTGGAACCAGATCTTGATCTGGCGCTCGGTGAGGCAGAGCG
CGTTGGCGATCTCGATGCGGCGGCGCCGTGTCAGGTAGCGGTTGAAGTGGAACTCCTTCTCCAGC
TCCAGTGTCTGGTAGCGCGTGTAGGTCTGGCGGCCTCGGCGCCCATGGCTCCCATACACAGCACC
TACGAGCAGAAACGGCCGGGCGCCG
CGI: 201 range = chr7: 27170441-27172987
CGGAGCTGGGCAAGCCGTCAGGGCGCCCTAAGGCCGCTGATCACGTCTGTGGCTTATTTGAATAA
TCTGTCATGGGGACCCTTGTGGCCCGGGTCGCCCGCAGCCTCATCTTGGCAGGATTTACGCCGCC
ACTGGCCGAAGGCAAGAAGTGGAAGGAATCGGCCGTCTCCCCCAGCGTCCCAGCTCCGGCTGCC
CTGGCTGCCGCCGCTCACGGACAATCTAGTTGTACAAAAGGCTCTCTGGGCTGCACTGCTTTCGA
AGAACGGCCCAAAGTATCTCGGTCCTGGGCCTGGGCAGCCAAGGAGAGGGGCGGCCAGTCTTGG
CTCGTCCCGAAGTGCCCGCCCCGCCCCCTCTCGCTGCAGCAGCCGCCTCCTCTCCCGTAGCCCTGC
GGGCCGCTCTTCACTGCTCTCCAGACTTGGGGCCCTATCTGAGGCGTCCCAAACACCAACTTCTG
GCTCCTGGCCCCAACTCGAGAGGCTTCCAGCGAGGACGAAGGCAGGCTCGAGAGAAACCTGGCG
GGCCAGCAGATCCGGGAGGCCGGCGTGGAGGCGGCGGCGGATTTGAAGGGAGGAGACACTTACT
GGGATCGATGGGGGGCTTGTCTCCGCCGCTCTCATTCTCAGCATTGTTTTCAGAGAAGGCGCCTTC
GCTGGGTTGTTTTTCTCTATCAACTGGAGGAGAACCACAAGCATAGTCAGTCAGGGACAAAGTGT
GAGTGTCAAGCGTGGGACAGTCACCCCTTCTGGCCGACAGCGGTTCAGGTTTAATGCCATAAGGC
CGGCTGGAGGGCAAGCCCGCGAAGGAGAGCGCACCGGGCGTGGGCTCCAGCCAGGAGCGCATGT
ACCTGCCGTCCGGCGCCGCCGCCGCCACGGGCGCCTGGGGGTGCACGTAGGGGTGGTGGTGATG
GTGGTGGTACACCGCAGCGGGTACAGCGTTGGCGCCCGCCGCGTGCACTGGGTTCCACGAGGCG
CCAAACACCGTCGCCTTGGACTGGAAGCTGCACGGGCTGAAGTCGGGGTGCTCGGCCAGCGTCG
CCGCCTGCCGGGGAGGCTGGCCCAGGGTCCCCGGCGCATAGCGGCCAACGCTCAGCTCATCCGC
GGCGTCGGCGCCCAGCAGGAACGAGTCCACGTAGTAGTTGCCCAGGGCCCCAGTGGTGGCCATC
ACCGTGCCCAGCGCCTGGCCCGCCCGGCCCGACCCACGGAAATTATGAAACTGCAGATTTCATGT
AACAACTTGGTGGCACCGGGGGGGAAGTACAGTCACCTAATAAGTTGCCGGCGCCCGCGCCCCC
ATTGGCCGTGCGCGTCACGTGCCCGTCCAGCAGAACAATAACGCGTAAATCACTCCGCACGCTAT
TAATGGTCCGATGTTTTGCAGTCATAATTTTTATAGCAAAAGCCATATGTTTTTATGTAAAGGGAT
CGTGCCGCTCTACGATGGGGTTTGTTTTAATTGTGGCCAACGACGATTAAAAGATCAAATCTAGC
CTTGTCTCTGTACTCTCCCGTCTCCCCCCCCATACACACACTTCTTAAGCGGACTATTTTATATCAC
AATTAATCACGCCATCAAGAAGGCGCGGGTCCCGCGTGCGAGTGCGGCCAGCGGAGCCCCTCAC
ATAAAATTAGACAATAATTGAAGCCATAAAAAAGCAGCCAAATCGCATTGTCGCTCTACTGTATT
TAAATCTATATTTATGATATTTCATAAGGAGTTATTGTTTCAGAAGCCACACAGGCTGGCGGGAA
GTCGGAAACGACCAACAGATTCGTTTGCCTCGCCGTGGCTCCCAGCTGTAAAAATTTACGAGGAC
TTGGAAAGGTTAGACTGTTGTGTTTGGTTGGCGAGCTCCCTGTAAATAATCCCTGCGGTCCCCGG
GAGAGGCGAGTTTACCCGCGGCCGCCCTCGAAAAGTCAAATTCAACGCAGGATCCGTCCCAAAC
GGAGCCGCCGCCGGCCCTACCAGGGCACTCCAGGCAGGGACCGGCCGCTCAGGGAGTACCGCGG
GTGTAGGTCCCCACAGCTACCCGCCTGGAGCGAGGGGCGCCCGGGCAACCCTTAAATTCGCCTTT
GCTACGAGGACCCCACGGAGGAGCTGGCCAGGAGGGAGCGGCCAGCCGCCACCAGGGCGAAGG
TTTTGAGGGCCTGGTTGGTTGTGCGGCGCGCTCGGTCCCCGGCCCTCGACCCCACGCACACGCGC
GCCCAGCCCGCCTTTCTCATCAGCTGGCAATCAGGATTCCCAGGCGCAGGCGGCTGGCGACCCAG
CCCTGTGCTCCAGCCTCAGAGGCTCTAACCATGAGCGCTGCAAGCCTGGTTGCGCTCCGTGAATC
CCAGCTGGGGAAAAAACTACAAGTGGCATGAATGGAAGGCAAGTTCGGTTTGGGAAAAGGCAGC
CTCGCCTAAGAGACCCCGCAGCTCCGGAACCTGGGAGGCCCGCACCGATGTGGCCTGTCCCGGG
GCCGCGTGAGCCTTTCAGGGCTCCTTCCTCCCTTTCCAGCTGCTACTCCGGGCCTCGCCTTGGTTA
CCTACGGGGCCCGGAGACTCGGCG
CGI: 114 range-chr7: 27198331-27199622
CGGCGGCAGCGATCGGCCCTGACCATAGAGGCGGCCGTGGCGCGGAGGACTTGACCTTTACGGC
ACGAGAGGAGGGCGCTGGCTGAGCCGCAGGGAGGGGGACGCTGCTCTCTCCAGCCTCCTCCACC
CAATAGCAGTCCGCTCGACCCACGCCAGCTGCGCGCTCACCCCCTTTCCCCCTCCATTTTGTGCAG
TGTCCGCAGACCCGCGGCCGGAAACAAAGCCCCCAGCCCTGCTCTCACCACCCTTCTTGCAAGCT
GCTGTCGCCAACCCCCCAGACCAGAGGCTCAGAGCTAACGCTAAGCCCCTTAAGGCCCTCTCCAG
GGCCCTCTTCTCTCGCCAGCCGCCCTCAGCCAGCTCCTGACCCCAGCCGCCCTCAGCCAGCTCCTG
GGTACCACTCTGGGTGCCAACCGGGACCTGCCGGTGCCACGGTCCCGTGCGCCGGGTCCCGGCCT
CGGGCTGCGGGCGGCTTCGGGGCCTACAGCCCAGCGGCGCGGAGCGTGGCAGAGGCGCGGGGCG
CAGCGCAGCCCGAGGCTGCCTCCCGCCCGGGCGGACGGCGCGGCCGCTGGGGTGCACAGCGTAG
CCCCGCGTGCCCGGCTCGCTCTCCGTTCCCTCGGATAGCTCCCACCGCTCGGCTCCGGGCTCAGAA
AGCGGAAGTATTTGGTAGAGAAAAACACGGTTTCTTTTCAGCTCGTCTCAAAATCCCTTTTAGAG
AAAATGCTCCTGTCAAGTTTTATTTCCCGTTGCAAACCACCTTCCACGCTGCCAAGAATTAAGACC
GGGAGAGATTAAATACCCGATTATTCTCCTGGGGAGGGCGGGGCGGGGCCGGGAAGTGGGCACC
CACACCAAACATTCCTTGAAGTAGGCTTGTCCTGATCCAGCCGCGCCCGGGGAGCCCCACGAAGG
CCCGCGCGGCCTGCGGTGACGTCAGCCTGCAGTTGCGGGGCCACTCACCCGCACAGACACGGCCC
TTGCTGTTCCGCGCCGGGACCTCCGCCAGCCGTCCTGCCGCAGCCCCGACTGGCCCGCGTGCCGT
CAGAGGGAGGCCCGCTTTACACCGGCCTGAGCCTCCATTTTGAGTAGGGGGTTCTTGTAGAAGCG
TGGAGGGTTTGGGGCCGCGCCTCTGTCCCCGAGGGGCGCGACATCAGGTAGCTCTCCGAGTTCAC
ACCCCAGTACCTGGGGGAGGACCTATGTCGCCGCATAACCGCCCGCAGAGGTTTGGAAATCTCTG
AGACCCGTTGTAATTATTTCTGCCCAGTGGATCCGGCTCTTCAGCGTCACGAGAGCCG
CGI: 111 range-chr7: 35263447-35264743
CGGTTTCCGGCTTTTGGACAATGGTCCCTCGCTTAACGAATGCTAACGAATGCTCCCTTAGCTGGG
AGCTGCAGGGACCGGTCCCGGCAGGTCTAACCAACCGTCCGCAGGCGCCGTGGTCGAGAGCAGA
GCCGAGACGGCGGGGCAGGGTGCCAAGCGGAGGGCACAGCCTGGCAGCCACCGTTGGCGGCAG
GAGAGAATGGGAGAAGGGAGACCCGCTTCTCAGCACAAGGGCATCTGTCTACTCCCGAACATGG
CCGGAGATTCCTGTCCACAGCCTCCCTCCAGCTCTTTCTGGTAGACACTTTAAAAGCGACATTAAC
GGGCTTCGGTGCCTTTGGATTCCTAAATTAGGTTTACGGAAAAGGAAAGACTTCCCAAGAACAGT
GAGAAGAGGAACTGCAGCCCCTCCAAGGCCGCTGCGGCGCGTTCCCGGGGCGCGCCGAGAGCAG
CGCGCGGCTCCGTGCCCCGTGGGGAGCGCGCGGCGCGGCCTTGGATTTCACCGCGAGTCGGGAG
GGCGGGTCTGAGCCTTGCCTCCCAGGATCCTTCCGACGAACACCCCGCGGGTTTTAGTTTATCGA
GCCAAAGTGGTCCCGGAGAAGCGCTCCCTCGCAGCCAAGCTGCAAGAAGTGGCCGGGAACCTAC
AGGCCTCGGGCCGACCCAGGAAGCCTCCGCACCAGAAAGCTCGAGGAGCCCTTACCCAAGTCTT
GCTCGAAGGGCAAAGCAAAGAGCCAGCACCCCTGAGTGTCACTGAAGTTCCTGGATGGGGTGTG
AGTGCGCGCGTTCCGTCCGAGACCTCAGTCTCGCCCAGCTATAGAGCCGATAAAGGGATGTCTTG
TGGGCGTAAGGCGCTTCGCGCCCATCTCCAAGGCCGATGTGGTCAGGAGGTGAGGGGAAATGTC
CTTCTGGCAGAAGCCCGCGGTGCTGCGACGTTGACCCGCCTGGCCTCAGGCTCAGGGCGGCGGGC
AGCCCAGGGCACATGTAGTTTCAGCAGCCGCGCTACGTGGGCGGGGGACCCCAGGCCACCCCAC
GTGTCCGCCCTGGGCCTCCTCCGGGTCCCAAGGCGCGGCGCCTCCAGGCCTTGTAGCGTCTTCCCC
GGGTCCCCGCGCGCCAGGCCCCGCAGCCTGCTCACAGGACAGCCCTCGGGGCTGCGACCCTCTCG
CTCCTCCCCGCGGCCACTCGCTCCCCGTTCATCCCCCACGAGCTCACTACCGCAGGGACCGGCCCT
CAGAGCCCCGGGCCTCCTCCCGGCAGTGGAAGAATCAGCGTGCTAACATTGTGTGCAAAACTCGC
AGCG
CGI: 30 range-chr7: 96469320-96469736
CGAGGGCCTTGCCCGCCCTCCTCCTCCCACCCGCAAGGGTTGGCCCGCGTTCCCCTGCGGGCGAG
ACAAAGAAGCAGGAGCCAGGACCCGGCTGGCGCCTAACCCGGCGCCGGGGCCTTGAGCCCCTGA
GGTGTGCGCCTTCCTGCAAGGCCCCAAAGAACCCGAGTTTTTGCCCCTTGCTGAAGGGTTGGAAG
TGCGGGCTGGACTAGTGCAAACAAGAGTGACTCCAACCGCCTTACTTCAGCTCATTACCGTCACC
CATGAAAACGGAATGAAGGACTGGGAAGCCAATCTGCCGCGTTCAATTGGAGAGAAATGACTGT
CGGAAGAGGCCCTGCGGTAAATAGCCTCTGAGCCGCGAACTCTATGCGCCGTGGCCGGTGGCAG
AGGCCACACCTGGCAGCCACTCGTGGCGCG
CGI: 32 range = chr7: 119702299-119702721
CGGAAATCATCGGCGACTGCTGTTATGAGGAGTACAAGGATCGCAGGCGAGAGAACGCCGAGCG
CCTGCAGGACGACGCGGATACCGACACCGCTGGGGAGAGCGCCTTGCCCACCATGACTGCAAGG
CAGAGGGTCTGGAGGGCCTTCGAGAACCCCCACACCAGCACGATGGCCCTGGTGTTCTACTATGT
CACGGGGTTTTTCATTGCCGTCTCTGTCATCGCGAATGTGGTGGAAACAGTGCCGTGCGGATCAA
GCCCAGGTCACATTAAAGAACTGCCCTGTGGAGAGCGGTATGCTGTGGCCTTCTTCTGCTTGGAC
ACGGCCTGCGTCATGATCTTCACAGTTGAGTATTTGCTTCGCCTGGCTGCAGCGCCTAGTCGTTAC
CGTTTTGTGCGTAGTGTCATGAGTATCATCGACG
CGI: 42 range-chr7: 158509937-158510390
CGTCCTCCGACGCGAGATGGCTGTGTGCCACCGACTGTCCAAATGCCCTGCCAATGCCATCGTGA
AGCATCCGTGTGAAGGAAAAAGTCCACCTTACCGTGCGCTGTGGCATCGATCAGGCGTGTGGACA
GTCGCAGGCACGCAGCGGTCTCCCAACATCAGGGCACACCGCGATCAGGTGTGTGGACAGTCGC
AGGCACGCAGCGGTCCTGCAGCAGCAGGGCGCACCGCGATCAGGTGTGTGGACAGTTGCAGGCA
CGCAGCGGTCTCCCAGCATCAGGGCACACCGCGATCAGGTGTGTGGACAGTCGCAGGCACGAAG
CGGTCCTGCAGCAGCAGGGCGCACCGCGATCAGGTGTGTGGACAGTTGCAGGCACGCAGCGGTC
TCCCAGCGTCAGGGCGCACCGCGATCAGGTGTGTGGACAGTCGGTGGGCACGCAGCGGTCTCCC
AGCG
CGI: 20 range = chr8: 10624024-10624296
CGCAGGAGGGCTCGGCCTGGGACCCCCTCTGCGGGCCTGCGGCTTTAGCCAGCAGAAAGAGGGG
CGTCAGTCCTATCCCCAAAGTTCCGCCTCGGGAAAGAGCCCTGAGCTCTGAACTCCGACGAAAAA
AGGAAGGGCTCTGTTCCGGACCGGGTGGGGTTTGGGAAGGGTAGAGCCGGCCTCGGGTGCCCTT
CTTACAGGGCCTGTCTCCCGCTGCGTGCACCAAGTGCTTTGAAAGTTTTTCGCGGATTAAAACGA
ATAAAAACCTCGTCG
CGI: 221 range = chr8: 11597006-11600365
CGAGGGTGAAGGATCGCCGCAAGGCACCGCACCTCCCGCTGCAGCCCATCCCGCACTACTAGGA
GAAGCCGGCGTAGGAGCGCCGCCTGTGTCCTTGGCTGTGGGGAGGACGTCAGATGGCACCCCGC
CAGACACTAAGCCCCAAGCCCCTGGCTTGTTGCTAAGAAAATTCACTGCCCGGTCCAGACTCAGC
CCTTTTCGCCCTTTAAGGGTCGCGCGTGGGAGGCAGCTCTGAGACCCCGGGTAGCGCTGGAGCCA
CAGATTTCCTCCGAGAAAAGAAAGGCCGGGATAGCTTCCCGCTCGCCCAAGCCCAGATTTTCCAC
TCTCCAGGAAGGCCTTGCAGGTCCCTGCCGCAGGCCTTGGCTTCGCGCCTCTCTCGCTCGCCCCCC
ACGAAGATGATTGCCGGTTTCAAACCGGGAGCAGGGAGTCTGCTTCCTTCTCCGCTGAGTCCGAA
GGATCGCAGATTGGAGCGTGCTCCGGAGACCGCTTTTCCGCAGCGCCGGCCTCCGAGATCCCCAG
CACCCCTTCAGCCTTAAGTTCCCACGTTTCGGGTCCGTGGCGCCAATTCTGCTAAGTAGCAGGCTA
GGAATTGGGGGAAGTCGGAGAAGAAACCCTAAGTGTGTCGCCCCCAGCTTCCGGGATGCAGGCC
CGCCGGGGTCTAGAGGGGCGGCTGCCGTGCGTCCAGCCTGTGCGCAGGCCTTTCGCCGCTCGGCG
CCCCAGGCAGCCTCAGTTTCCTTTCCTCTGTTTGCGCCCCAGTGAACCTCCGCACCTCTCATTCAG
GGAAGAGAATTCCCCGCGCAGCCGCGCTCGTTTCTTCCTCTGGGATTTTCCTGAGAATCCCCAGG
AGTTGGCCACGATCCCATGGGGGGTTTCCTTCTACCCAGCCCCGCGTCCTGGCCTCGTCCTTAACC
CCCGGGTTGCCTTCACTCAGGCTGGGAATCCACGATTGATTTCCTACTACGGAAGCGGGTGGCGT
TCCCAGCCTGCTTTCGGAGCAGCACGGGTTTCGTGCAGGGTGTTATCCCGACCCCTTCCCCCATCC
CTCTAATCTGGCTTGAGAAGCCCGTGCTGGAGAGAAAAACGCGGCCTTAAAAAAAAAAAAAAGT
TTAACCGAAAGCGTGAGAGCCACCCGCCGGCTGTTATCTGGGGCTGAAGGCTGCGGTAATCGATG
GGTTATTTTTACGCGGTAATAGGGCCCTGTGATTGCTCTATTAACCTTTAGACCTGTCTGAGGGAC
TCTCCGGCTCGCAGCCCCGCTGCGCTGGGGCCTCCAGGCTCTGACGCCGACTCCCAACTCAGGCC
TGACACATTCCCCTCCCCCATACCCTGGAAGAGCCCCCTCCATGAAGAAGCTCCCCTGGACCGCC
TGGCTCCCCAGCCCTTGCCACGTCCCTTGGATTGGTGCAGAGCCGCCGCAGGCTGCAGAAAAAAG
GGGGAAAGATTAGAAGAGAGGAGGCCACAGGAGATGGGAAGTGTCGCCAGGAAGGGATGCAGA
TTGCATAAATACATAAAATTGAGGCTGAGGCCTGGGCTCCCGACCATCTCCCTGGGATTTTGGGA
AGGCAAAAGGGAGGCTTCGGTCTCTACGCTCTGATTTTAGGAGGCAGTCTGGGTGTCTCCTGAAC
CTCCAAGGAATCCGGGGCTGGGAGGATCCCCACTACCCCTGCCCAGGAACTAGCATCCAGCCGG
GCACCCCGGGTGACCCAGTGCCCCACACAAGATCGAGAGTTGAGCCCAAGAGGTCACCTTCTTCT
CTACTGGCCCCGCCCCTCGCCCGCCGCTGCGGGATGAGGACCACAGGAAGGGGGGGGGGGGAGG
GAGAAAGGGAACTCATTAATAAAGCTGACCCTGGGCACCACAGCGAACCCAATCGACCTCCGGC
TGGGTTGCGGGTGATTCCCCGCTCCCTGGCGGTAGCACTTGGGCATTTTCCGCGGAGACCCCAGA
GCCTGGACTTTGCCTGCTGGGGGAGCTTTCCGCACAGTCCCGCAGCCTGCGCCCAGCGGAGGTGT
AGCCGGGGCCGCGCACCCCCGCCCCGCCCTTGCACGTGACTCCCACAGGCCAGTCAGCGCCCTAG
GGCCGAGTTGCTGGGCCGGGGACCCGAGCCGCGAGCTGGGGACTTGGAGGCGGCCGGCGCAGGG
GCCGCGAGAGGCTTCGTCGCCGCTGCAGCTCCGGGGGCTCCCAGGGGAGCGTGCGCGGAACCTC
CAGGCCCAGCAGGTAGGGCTTTTTTCTTCCCTTTCTTTGCTCCTTCCCGCGGTCCCCCAAACTCGG
AGCTTCTCCGCCTTTGCTTGTCTGGAGGTAGAGAGGTAGCTAGTGGGAGGAAAAGAGACGTGCGC
TACTCACTTCACCGAAATTGCCCAACCCCTGCTCTGCTTTTGACTTTGCCTTAGCAACTTCTTTAAG
TCAAAGTAAGACTTGGGGGCAAAACAGAGAAATATTGGAAGCGCCTTTGGATTCTTTCCGTGTGA
ACTTGAACGCTTTCAATCCCTGTCCCCGTGTGCACATTCTCCAACCCTTGTTTGCATATCGCAGGC
CGGGGCCTGGGTGGTGATGGTGGCCGCGTGAAGTTACCGGGACTGACGGGCCCGGGACAGGCTG
CACGGCAGCTCGCACATGGAGGGAAGTAGACGGAGGCTTGTCGCCCACCAGCGACTCCGGGGAC
GCAGGGTGGCAGTGCCAGGCAGCTCCGCTGGGCCTCAGGGGCCCCCGGGAGCCGCTCTGAGGTG
CGGAGAGGCTGCTGAGTGGCGGAACTATTCATGCCCTTTCTGGCCGGCCTCCTCGCCCTCGGGGC
TGGGGTCCAGGGACTGAATGCTCCTCTGGAAGCTCACCACCCCACCTGCCCGCGCTGCTTCTACC
TGAAACTGGCCAAGGGCCCGAGCCCGGACCGGAGCCGTGACTTCCCTCCGCCGGCCACGGGGCT
GCCCGGATCCGCCGGGTTATGTCGCTTGGCTTTGGGCTCAGGGGTCACCGTGGGCAGAGGGGGGT
GCCGGGGTCGCGGACTGCCACCAGGTTGAGGAAAGGAGGGGCCTTTTGGCTGGGGAAAGAGCGT
GGTGGGGGACCCGCGGCCGATGGAATCCCTGGGGCAGCGCGGCCCGCACCGTGGAGGTTGGGGA
AGCGCCTCGGGGAAGTGTTTCCTGTGTTCCCAGAAAAGGAAGACAACCGAGAGCAGGTTTCAGG
CTTTTAAAGAAAGCCTGGGGTGTGGAGGTGATGCTCCGCACACGTCTGTGTCTCCTCCCCTGCTGC
GGCCGGCTTGGTTGTGCCGGCTAGCGTGCGACCGTCCTCCTCGCTGCAGGCCGAGAGCGGAGGCG
TAAACCCAGGCCAGCGAGGAGTGTCCTATAAAGGGACGGGGACTTTTCGGCG
CGI: 25 range-chr8: 25955253-25955609
CGTTTGCTCCGCCGTCAGTCCATAACCAGGCGCTACTAAGGCCTCCAGGCTCGGTGCGCAGAGCC
GGGAGCGCTCCTCCAGCTCCAGGTTGGGGGAGTCGGGGTGGGAGGAGGTTCGGGAAATCGACTG
ATTTAGAGGCTGGGGGTGAGTAGCCTCGATTTATCATCCCTGATGGTGAGAGTGGAAAAGTGTCG
CGAGAGTGACAGATGGAATCTTTAGTGCTTCGCAAGAGGGAGTTATTAGGCCGCGGGGTTTGGG
GGGTACTTTCCGCTTACTTTGTCATTCTCCACAAAGTCCACGAAGGCCGTCCGCTCGATCTCCACC
GGCTGGCCCTGCCTGTCATAGAGCGCCAGGACG
CGI: 233 range = chr8: 55532724-55535078
CGCTCGGTATGTTCATCTAAACGACCTTGGGCAAGTACGTCGATTCCAAGGTACAATCAGCCCTC
CCAGACGGCTTTTCGAGTCTCCCTAACCCCGGTGGGAGAGGACGCGGCGCCAGAGCCCAGCTCCG
GCTAGTTTTCCCGGGGGCAGGTGTAGCCTTGGGCGCGGGGCCGGGGGAGGGGCAAGGGGCGGGC
GTGGGGTTGGACTGGGACGTGGGACTCGGACCACGGCCTGGGCGTGGGCCTAACGACGCGGGAC
CGGCCCGCCCTCGCCGCTCCATTGGCCACATCTGTGCAGAAAAGGCCCCGCGGCCCAGGGGCGCC
CGCAGTGTCACTAGGCCGGCTGGGGGCCCTGGGTACGCTGTAGACCAGACCGCGACAGGCCAGA
ACACGGGCGGCGGCTTCGGGCCGGGAGACCCGCGCAGCCCTCGGGGCATCTCAGTGCCTCACTCC
CCACCCCCTCCCCCGGGTCGGGGGAGGCGGCGCGTCCGGCGGAGGGTTGAGGGGAGCGGGGCAG
GCCTGGAGCGCCATGAGCAGCCCGGATGCGGGATACGCCAGTGACGACCAGAGCCAGACCCAGA
GCGCGCTGCCCGCGGTGATGGCCGGGCTGGGCCCCTGCCCCTGGGCCGAGTCGCTGAGCCCCATC
GGGGACATGAAGGTGAAGGGCGAGGCGCCGGCGAACAGCGGAGCACCGGCCGGGGCCGCGGGC
CGAGCCAAGGGCGAGTCCCGTATCCGGCGGCCGATGAACGCTTTCATGGTGTGGGCTAAGGACG
AGCGCAAGCGGCTGGCGCAGCAGAATCCAGACCTGCACAACGCCGAGTTGAGCAAGATGCTGGG
TGAGTCCGAGTCGCAGACCCAGGCGGCCGGGCGCGCTGGCGCGAATCGCTAGGCCGATTTCTTAA
ACCCCAAACTGTTCTTTGCGAGCCTGACGCCCAAAACCAGGGGTGTGTAGCGGCCACGTCCTTTC
TTAAGGCTCTGGGTTCCCTTCCCGCTTCCCGCCCTCCGACCCTCCAAAGCAGCTTTCCGCCTTGCT
CTCCGGCTCCCGGATTCCCCAGGTGGCCGGGGGCGCGGGTCCAACGGCTCTGGGAAGGCGACTTC
CCGGCACCTCCGGGCGGCGCGAGAGCACCCTTGGCCCTGAACTGGGCCGGTTGTGTCCATCCCTC
GACCCCTTCCCTAGTTAGGTGTCCTTTTCTGTTTTTCGAACGACCGGGTGATGGGTGAGCGGAAAG
CCGCTTCCAGGAGACCAAAAGAAAGGGGTGCCTTTAGAGGACGGGTGTTCCCCAAGGGCTCGGA
CTCAGGAGTCCCAGATCTCCCTCTTTAACTTCACCCCGGTTGCGCAATTCAAAGTCTGAGGGGGG
AGGTGCGTCCAGGTGGGGCCAGGTGGGGCCTGGAGCGGGAGCGCAGCCGATAAGCCCTGCGCCC
CTCTCCCCCTTCCTTCCACTGTGCAGGCAAGTCGTGGAAGGCGCTGACGCTGGCGGAGAAGCGGC
CCTTCGTGGAGGAGGCAGAGCGGCTGCGCGTGCAGCACATGCAGGACCACCCCAACTACAAGTA
CCGGCCGCGGCGGCGCAAGCAGGTGAAGCGGCTGAAGCGGGTGGAGGGCGGCTTCCTGCACGGC
CTGGCTGAGCCGCAGGCGGCCGCGCTGGGCCCCGAGGGCGGCCGCGTGGCCATGGACGGCCTGG
GCCTCCAGTTCCCCGAGCAGGGCTTCCCCGCCGGCCCGCCGCTGCTGCCTCCGCACATGGGCGGC
CACTACCGCGACTGCCAGAGTCTGGGCGCGCCTCCGCTCGACGGCTACCCGTTGCCCACGCCCGA
CACGTCCCCGCTGGACGGCGTGGACCCCGACCCGGCTTTCTTCGCCGCCCCGATGCCCGGGGACT
GCCCGGCGGCCGGCACCTACAGCTACGCGCAGGTCTCGGACTACGCTGGCCCCCCGGAGCCTCCC
GCCGGTCCCATGCACCCCCGACTCGGCCCAGAGCCCGCGGGTCCCTCGATTCCGGGCCTCCTGGC
GCCACCCAGCGCCCTTCACGTGTACTACGGCGCGATGGGCTCGCCCGGGGGGGGCGGCGGGCGC
GGCTTCCAGATGCAGCCGCAACACCAGCACCAGCACCAGCACCAGCACCACCCCCCGGGCCCCG
GACAGCCGTCGCCCCCTCCGGAGGCACTGCCCTGCCGGGACGGCACGGACCCCAGTCAGCCCGC
CGAGCTCCTCGGGGAGGTGGACCGCACGGAATTTGAACAGTATCTGCACTTCGTGTGCAAGCCTG
AGATGGGCCTCCCCTACCAGGGGCATGACTCCGGTGTGAATCTCCCCGACAGCCACGGGGCCATT
TCCTCGGTGGTGTCCGACGCCAGCTCCGCG
CGI: 74 range-chr8: 72916429-72917309
CGGGGCAAGGGCGCGTTCCCTATCGCAGGATCACTTGCTATGGTAAGCCGCCCACCCTGCGCGCT
CCTCCGCGCGGGGAAGAACCTGCGCGGCAGGACGTGGTGTTGGAGTTGGGGCGCCCGGAGCGTG
GAGTGGGGAGACCTGATGCAGAGAGTCTGGAGTCGGAGCTGGGGGTGCTGCAGGTAGGAGCAGG
AGCGGGGCGGAGAGGGAGGCCCGAAGAAGACCCCACACAGGTTGGCGCAGCGGGGCTTGGGGA
GGCTTCAGCCCAGAAGTGGAGAGGGTTGACAGACGCCTGCCTGATTAGAAAAAGCCGGGAGCTT
GGGAAGGAGACGGGATTGAAGAAGCCACCCGGCAGGGAGGCCGAACGGCCCAGAGCTCTCCGG
GTAAAACCCGCCTGCGGTGATCTGGGAAGTGTGTCTCCACCTAGCCCTGCGAGCAGCGGCCTTCC
TCCCGCCCGTTAGAAGGGCGCTGTGCTGGAGTACGAACCCGGCCCAGAGAAGCCACTCGCCCTTC
TTTGTCACTTAAAACCCTGTCCCGACGCGGATCTCACGTCTAGACCTCTGTCTTTAAAGCGGATGT
AGAGCGCGTTCTAACCGTTCCCTAACCATTGTGTCACCGCGAAAGGCCGGGGCTGTGTGGAACCG
TCCCGCACGTGTGCGATGAATCTGGCTCCGCTGAGAACGGATCCGTGGGCTGTCTGGCGCGGGCT
GGGGCAGCAGCCGAGAGTTAGTCTACAGAGCTAGGGCCCGAGGGTGGACCTGCGTCCGCGTCTC
GTCACGAAAGGAAGCTCTTTTTGGAGAGGCAAAACGTGGTCGCCCAGATCCGGCGCAGCTGTAG
CCGTGGGCGCTGTGCAGTGACAGCCACACCCGCCACCTGTCACG
CGI: 17 range = chr8: 140700279-140700514
CGGCCGCCCCGATGCACAGCGTCCCCATGCAGGAGAAGAAGCCCACAGTCACCATGTTCTCCATA
GACACGTCAGTGTTGCGCATGCCACAGCACTTCTTAATGCGCTTCAGCAGGTAGCGCACGAAGGT
GTTCATGCGCTCGCCCAGGCTCTGGAACATGACCAGTGTCAGCGGGATGCCCAGCACGGCGTAGA
ACATGCAGAAGGCCTTGCCCGCATCGGTGCCAGGTGCAGCG
CGI: 21 range = chr8: 145014900-145015187
CGCTGACCACCTGCCGCAGGGCCGCAGCCCTCCCAGACCGACAGAGTGCCACCAGCTCCCGCCGC
TTGTCAGCGCCAACGTATTCGGAGAGCAGCAGGTCCCAGAGTGACACGCTACACCCCTTAAACCT
CCCTGTGCTGACGGGCACCTTCATGGCCCTCAGAGCCACCGCGGTGTGGTCGTCCACCTCAAGCA
CTGTGTCTGAGGGCAGTGGCAACAGCAACAATCCGGTCTCGGAGTCGCACACGCAGCGCTCCCTG
AGCTGCTGGTAGGTCACCTGGTCCCGCG
CGI: 260 range = chr9: 959530-963276
CGTGTGCCCTTCGGCGGGCGGCTGTAGCTGTAGCTGTTGTGACGGCTACGGCGGAGGCTGCGGCC
GCGCGGGGAATGGAGCCGGACCGCGGAGTCGTCACCTCCAAGGTGTTTCTAGTGGCCTCCTGGAA
GATGATCCCGCGCCCACCTTGCCGGCGTGTTCGCGGGCCCCTGCCCACTGCCCCCCCTCTTTCTTT
AGGTCTGGCTTTTGAGGATCCCGGAGTCTTGCAGCTCCCGCCATTCCGCAGATAACCTCCGCACA
CTTAATTGTGACCCGCGGGGTGGTCGGAGAAGCTCCGCACGCGTCCTCAGTGGGAAAGTGTCCCC
TCTCAGCACTGCCCCCTTCAGTCCCCCTGCATTTCTGGAAAGTCAGGCAAGGCCCAGGTAGGCTG
CCAGTGCACTCTCAGCCTTGACTGAGCACCTTTCGGGCCTCTGGTGAGCCCCGAGAGCCAGGTTT
GGCTCGGCAGAGCGGGCTTGGGCTGCGCAAGCAATGCGCATCGTGGCCGCCTGCACCCTGGGAA
CTAGGCCTGTCCAGTGGGCAGCATCCTCATTTTTGAAAAGGCCCTTCTAAACCCACCGGCCTCGC
CTAACGCCGTTTGGTGCTGCATCCGAGCGGCCTCACGCGGTCCCCTGGAAGGGCCACTCCCAGCG
GGAGGGCGGGGCCCGGGACCTGCGCTGGCCACGCAGGGTCTCAAGCTGACCGGAAGACCCGGCT
TTTGGCCTGTGTCCAATAGCCCCAGAAGAGAGGGGTCTGGAGCCTTCCTCCCACGCGTAGTGACG
CTCAGGTGTCCTCGGGTTGTTAGTCTTGACCCAGGAGAGTGCAAGACAGGCCCAAGGCCTGGGGG
GATTGCGTTATGAATGTCCAATTCTAAACACAAGGTAGAGCACAGACAGTACATCCTCACGGCTG
GATTTTAACCGTATTTTAATAAACACATTCGAGGGGGTGTCAGTTTCCCCAAGCTCTGCCCCCTTC
CGCGGGCGGGATCCATGGTGTGTGCAGTGTAAGAGTGCGCAGAACGCGTGTGTTCAAGTGTGGG
CGTGGCAGGCGTCGTGTGCTCGCCCCGCGCACTGTGCGGATCGCCCAGACAGCCTTGACAGGTTT
TTGCAGATGTTTGGGTGCTACGGTGTGGGGAAACCCAGGCAGGAGCGCCAGGCCTAATTCTCCTG
GACTCTTGGTGAGCGGCCGCTACTCCACGAGGGGCTAGAAGCAAAGGGGGCACGCGCTTTTCCCC
AGGCCGCCTCTTGCTGCCGCAGTGGCTGAGGGCGCTGATGACCCCTCCCCGCTTCCAGCGGACTT
GACCCGCGGGCTGACAACCCACCGCGACAAGCAGGCGGCTGGGTTCGCGCCGCCGCCCCGGGGC
CCTTGGCTCAAATTTCACCTCGAGTCCTGCAGACCCTGCGCCACTGAATTGGGGCCCAGGACGCC
CTTGGTGACACTCGCCTTCTTGCTGCCACAACCACCGTCATACCCGCAGCCGGGGCTCCCTCCGCT
AACCACGCTTGGAGACCCCAATCGGGGACAGAGGTGGGAGTCAGACCCCCCCTGGCCTGCACTG
CCGTTTCCCTCGATTCTTGCGGAAACAAGACTCCCGCCCACACATAAAAATGCAGCTCCCGGCCA
CCGGGCGCCGGTGGCTCACGCCTGTAATCCCAACACCTTGGGAGGCCGAGGCGGGCGGATCACTT
GAGGTCAGGAGTTCGAGAGCAGCCTGACCGACTACTAAAAATACAAAAATTAGCCAGGAGTAGT
GGTGCATGCCTGTAATCCCAGCTACTCGGGAGGCTGAGGCACAAGAATCGTCTGAACCCGGGAA
GCGGAGGGAAGCAGCGAGTCGAGATCGCGCCACTGCACTCCAGCCTGGGCGACAGAATGAGATT
CCGTCTCAACAAATAAATAGAAATAAAAATATGCAGCCCCCTCCGCTCCACTTGAACTTTAATGC
TGAACCGGTTTCCCACGTATACGTGTATCGCACCGCATTTTGACGCTTTGCATCGAGTCGCATTAA
TGGCGCTTTTGAGAACGCGTCGTCGCGCTTTACAGAGAAACCCTACGGGCAGCCTGTGGAGGGGT
AGGGGATATTCATTGGCTTTCCCTGCTGGGCCCCGTCCGCCGGGCGGGTTAGGGTCGTGGCAGCC
TGCCCGCGCGCCGCTGACTCTGGAATTTTGTCCGGGAAACTGGCGTAGGGCCCTGGCTCTCCCTTC
GCCCTCCGCGCACACGCGGACGAGGCCTTAGATCCACAGCCTTTTCTAGGCCCTGCGCCTTTGAA
GCTGGGCCACTGCCAACCGCTCGCGATTCTCACCTTCAACAGTCGCCCCCTTACCCCTCCCCCACC
CGCCTGCCCTCGGGAGCGGGTCGCCTCCACTCCACCACCTGTTTAAGTTCCTCCCCCTCGGCGCCC
CTCCAGTCCCCACCCCGGCCCCGGTCCAAAAAACCAGCAAACGGAACTTTTCCACAGTTGAAAGC
CGCGGCCCGCGAGGCCGGGCTGGGAGGGGAAAGCGGGGCGTGTCTGGGGGGCGGGGCCCCGAG
CACTCCGGAAGTTGCCCCGCCCAGGAGGCTCCTGGGAAAGTGAGGAGAGGGCCCGGGCCTACTT
TCGTCCTGGTGTGGCGCCCCTCAGCCTCCCCTCCTCCCAGTTCCCGCGCCTCCGCAGGGCGCCTCG
GCCTGGCCTCCAGGCAAAGTTCGCGCCCCCTGTTCCTGGGGTGTCGGCCGCGCGGGCCGTTTCCC
TTCATTACTCCCGGGCCCCTGAATCCGAACGCTTTCCCAGAAGCGCGCAAATCCGCTTGCTTTCCC
CGCGGCTGGGCTTTGTTCAGGGACAGCAAAGGAGGAGGCGGGAGGCTGGTGAGGTTTTCTGGAA
AAGGGGCTTGTCCCGAGGAGGAAGTGCCCCAGATCCCTGAGAGCCAACGCTCTGGGGAGAAGAA
ACTTTCCTTCTCCCTTGAATGTTGCTCAGATTACCTAAAATTATTTTTTCAGCCCTTGTGTTCTAAA
GTCGCAGGGTAAAGGTTATCTTAAGACTTAACATCAGCGCTGCTCATTTGTACGTTGGTGGAGAC
GTGCCTTTTTTCCTTTGCACTTAAGGTGGACAGGGTCTGCGACGCTCCCTTCCAGGACGGTGTGGG
GAAGCGGCCGACGTCCCCAGCCGGACTCACGCCCTCCTACTACTGGGCGTCGGCTCCGCCGCGGG
CGCTCCCGACAGGGAGCTGGAGTCGGACGAGCGGCTGCCCCCAGGGCCTCCAGGAACCGCGGCC
CAGCGGGGAGCGCCCCAGGCTAGCGCTTTTCCAGTTCCCTTCGAAAGCGCGGGGCTGAGGTCGCG
GCGCTGGGCCCTCGGATGAAGCCGTGCTGTAGCTACACCTGAACCCCGCGAAAGGCTGGCGCGG
TCGTGTATCCAGGCTGGGTCTGAGGAATCCGCAAGCGGGAGAGCGCTAACTCCTAGGCGTGAGC
CGCTGCTGGCTTCGAGAGTTCGAGAACATGAAGGACCTGGCTTCTCCCGCCCGGTCGGCTTAGGG
CCAGCGAGGTCACAGGCCGTTCTGCTCTCCCTGTTTGTCCCCAAAGGCCTCGGCACGTGGGGATC
TGGAGCAGGCCTCAGGCTGCGACCCGTCTCTTCCCCTACCAAAATTATGTGGGAACAGCGGTCCA
GGACCTTCCCCTGTTCAGCGGTATCCCCGGGCCGGTGACCCCGGGGTTCAGTCGTCTCCCCGACC
CCAAGCGGCCTCTGCTTTCCACCCCTCGCCCCGGAGGGCGGCTTCG
CGI: 25 range = chr9: 21979774-21980108
CGCGGCCAGTGAAATCCCAATCGTCTTCCACGTGGAACCCCAGGTCCGCAGTTATGATAACGGAT
CACATCGCTCCTGCGGAAAGTGCGCGCGGTGGAGTGATAATTGGACCTAGCGTCTAAATTCTTGT
TGGAGGACCTCGTTCCAGCTGCCAGTTAAGCCTCTGGGATCCGCAGCGTCTCTAGGAATTGAGAG
AGTGGGGAAGTTAGGATCCAGGAGGAGGATGGTGGGGGCTGAGGAGTGGAGGAGCAGCGTGCA
TCTCATCTCTTGTCGCCGGGCGGGCGCTCTTTCGGGTCCAGGGCCCTTGCACCCCCAGCGTGGCTC
CGGAGGCGGCG
CGI: 20 range = chr9: 23812413-23812667
CGACTATGCAAACGCTTGCCGCTAGCCGCATCCTTCTATCGCCCCCCAACTCAAGACCCGAAATG
GCCAACTCTTCACCGAGGGGGCGTCCCATCGCTGCCTTGCCTTGGCCCGCCCCTTCGCCAAGCTCA
ATCCCTTTTCCCCAGAAAAACCTGTGCCTGTCCTCAGACCCGGCCCCACGGCTGGAAGTCAAAAC
CCACCCCGGAGCCACGCGGCTGGAGCACGTTTTTACACCTGCTCGGCCGCTTAATTACG
CGI: 278 range = chr9: 37024136-37028341
CGCTCACAGGTCGGAATAATTCAAGCCTTCCGCTCCCCCGCCGAGCTGGGGTAGCTGATCACTGA
GCTGAAACTAAACGTTTTAGGTGGAAAAAAAGCGTCCGAAGGCACCGTGAAATGATTAAGGAAC
TAAAGAGCTTCTCGCCATGTGAGATCATGTCCTGTTCTCGCCAACATCACAAGATGTCCCCAGAC
ACGCCGCGCCCCCAGCGCGCCGCCCCACACTGCCGGCCCGGAGCGAGGAAAGGGTAGGCGCTGC
GCGGCCGGGCCTGCTCAGCGCGCCAGACGTGGCGGACCCGGCCCGGCCGGAGTAGAGCGGGAAG
CCGGGAGAGCAGCAGTGCTGCTGCCGCGCCGCCCCAGACTTTTATAGGGGTTGGGGGGAGGGAA
GGAAGGCTTCAGCCTGCGCCGGGCGCTAGCCAGCGCACCTACGGGAAGGGGCAGACCGAAGCGG
AGCCCGGGAGCCCGGGTCTTCCAGCCGCACCTTCTATTAAGACATGCGCTTGGGGGTCGCGGCCC
TCTGCGCTGTGCGCGGGGCCTGTGACATCTCCATGTGCAAACCCCGCTGCTTAGACGAGCGCAGG
CCGGGTTTTTGTTTTTGAAGAAATTAACTGCTCGGCCAAGCAGGTGCTTTTGAAAGGGGTTGCAA
AGGCTGGCAGTGTTGCCAGGCACCGAGGCGACGTTCCGGTTTACTCCGATTCACCCCTAGGGCGG
CGCCCGACCCCAGGGTTCCCAGCCATCCAACCCCCACCCCAATTTGTTGGGGTCCTGACTATTGCT
GGGCCCCTGACCCTAGGGCAGCGCCAGATCCCTAAAGCCCCCAGGTCCCATCCAGTCTTCTAGTC
CCAGAACCCCTCCCCACATCAAGTTCCATTCTGGCCCAGAGCATGGAGGTTAAGCTGTGAAATAC
TGGAGATGAAATCCGGGCTTCCGCTTTGGAGCACCCGGGCAGAGCAGAACATCTCAGTAGTTTCT
GGAGATTGGCGGGAGTTCCAGACACCTTACCCCGGCCCATAAGTGGAATCGCATTGCCCTCGAGC
CCTGAGACGTTGTGGAGTCCCAGAGCCCCAGGACTTGATTCCAGAGAGGAAATCAGATCCACAG
AGACCGAGCAGAGGTGTGGCATGTCCAGCCCTCTCTCTTCGGGCCAGACTCAAGCCTCCCCTCCC
TGTTCCCCTCCCCAGCCTTGAGTTAGTTCAGCTGCTGGGCCTAGCCACTGAAAGCCTGTGGGGCCC
TGCAGCGGGGTGGTCGCAGGGGAAAGGTTCTCCAGGGCGGGGGAGCGCTCCGTAGGAGGACTCC
TGGCCCGCCGGACAATGGCAGTTTTGGAGAGTGAATCGTCCTCAGCAAGAGTGTTGTTTGTTTTTT
GGAGGGGGTGGTCACGAGGCTGGGTCGCAACCTAGGGGGAAGAGCCTAGAGCTCGAGGAAAGG
GGGGCCCCCGGCGGCAACTGGACATCACATATCCACTGAGCAGATTTCGCCACAATCGGAAATGT
GGCTGTGGATTCCCGGGCGGCCCCCCCACCAAGTGGTCTGCGCAGTTCCCCAGCGCCGCCTCCTC
CCTGCAGCCTGCGGGCCCGTGTCCCTACCCCGAGATTTGATTTTTGGCCATCTCCGAGTGCTCGAC
TCTTTCCCTCCGCAGCCCAGCCCCAGCCCAGGCTTCCCGCCCTCCCGCCTCGCCCGCGCCGGGAG
GCAGCGCCTGGGAGGAGCGGCCTGTCCGCGCCTGGAGATCAAAAGCCCTCCCGGAATTGACATTT
CACCCCGGGCTGGGGGCGGGGGGTGTTGGTGTTTTGTTGTTGTTGTTGTTTCCATTTAGAGGGCTT
GTTTTGTTTTTGTTTGTTTTCTGTGAGTTTTCATTTTTCCATGGGATATTCTTTTGTTTCGGGTGTTT
TGCGTGTTGTTTTTTCGTGGGATTGATTTTAGCGGGTTTTGCTCTGTAAGTCCTGGTCTTTGGGGTG
AGTTTTGATTTTGTGTAATTTTGTACTTTGTCCTTGTAGGCTGTCTTTTTGTGTGTCGTTTGTAAGG
CTTTTCCTTCTCCGCGGTGTTTAGATGTGTTTTGATTTGGTGTGAGTGGGTGTCGTCTGTGTGTGTG
TTGCCTTAGGGAATTTCGTGTTGTGTGTGTTTTGTTTCCTTCGATTTGAATTTTATTTTTTTGAACTG
TACGTTTTGGTGGGGGATCGGGTTATATTTTCATGTGTTGTCTTTAGTTTTTGTGTTTGTGAGATGC
GCTGGTGAGAGTTTGCGTGTGTATTTTGGCTTTTGTGGGGCTTTGGGATTAGGGAGGGGACATCA
GCCGCTACTCCGTTCCTGCCCGCACCCATGAAGGGCAGAGGATTAGGCCAAGCATCACGAATCGT
AGGTTCGCCGTGCTCCGCCGCTTCGCGCTCTGGACGCTCGAGACCAGCGGGAAACAAATGTGGTT
CCCGAGGGGGCGCCCGCTGGTCGTCAAAACCTCGCGGGCCCGGAGGCGCCGCTGCTGGGTCAGA
ATCGCAGAGCCCCCCTCGCGGGGTACCGAGGCCCAGCCGCCGTCCCCTTGCCCACCCTCTCCCCT
GTGAGTCGGTCTCCCCTTGCGCCTGGCCAGCATGCTTCGGACCGTCCGGACTCCCGCAGCGTTGG
CCCAAGGGGCGGCTCCCCTCGGCGGAGCAGGTGACGAACCACTTCGGAGGACACGGAATTGACA
CTGCGGCCCTAGAGATACGGGACAAGGGGCCAGAACGCGGGGAGTCTAGGCTCAGATCGGAGTC
GGGATCCCGGGAAAATCCCCCATCTCCCTTGCCCGCAGCCCTAGAATCCCAGAAACCCAACTCCG
CGCAGCTTTCGGTTTGGAGAGCCCGCCCTGGTTTGCAAGCACAGATACATGTGCCCACACCCGAA
CACACGCAGACACCGCCAGAGAGCGAGACAGCGAGCGGGCGCCTCCGTAGCAGCGGCCTGCGGG
GCGGAACCGGAGCTGGCGCCGCCTCAAGGGGAAACCGAACTGTAGTGACCGGAAATGTCCCCAG
GACAGAATAAGCCATTCCTTCTGTCCACGTTGCCAGGACTTCTCCAGCTCAACACACCTTGGTTTG
TTTAATGTTACACGAGGGAGAAGATTTAGAGGCAGAGACACATGCTGGCTCAATCGGCGAGGAA
ATACGAGCAGAGTCTAAACAGAAAGAAATATGGAACGGGGGGAGACTAGACTGTGGGGAAGGC
AAATTGGAGGCCCAGAAAAGGGGCTGGTGCGGAAGCTCCTCCAGGCCCCTGGGCGCTCTGGCAT
TCTGTGAGAAGGAGAAAGCAGAAGCCGAGAACAGAGAGTGACAGGAAAAGAAAAGAGACGCAG
AAGGGGTTGGCCTAGGCACGCTGCGAGCTGCCCTCCAGCTAAGACGCTGCGGGCAAGGGAGGGG
GAGGCCGACATCGCCAGGCGAGGGGCCAAGGTCGGGCTCTTTAACTCAGCCAGGAAACATCGTT
GGGGGCTCCAGGGCTCGTGGAGCCCGAACTGGGGCTCATTTCCGGCCCGATGCCCCTCTTCCCCA
GACCCCTAGCCCGGAATCCAGCAAAGGCTGGGCCATCGCCGTCCGCCACTGGGCCTCCCTCTGTG
CGAGCCCTGGGAGAAGCCTCAGGGAACGACTCTGGCACTGGGGCTCGGCCCCGGAGCTAGCGCA
GAGCGTGGGTGGGAGTCGACATCCAGGCAGGAGGCAAAGAAGAGGCCCCGCCCTAAAGCCGAG
GGCCCAGTTCTTCGAAAGCCTGTGGCCGGAGCTGGACTCACAGGATTCGGGGTCCGTACAGCGCG
CACGTGGTACCGCATCTTCGGCAGGCGGCGCCCCTAATGCCCAGCCCCGCCCTGCTTCGTTTGCC
GGGCCTGAACTGTGTCAGCATTGAGGGTGGACGCCGAGTCGCGGCGAGTTGGTCCCTTGCGGTAT
AAACTTGGGGCTTCCCTAGGGGCGGTGCCGGGATGGATGGGGGCGCCTCTCCACCTGCAACTCCC
CCAGCGCAGAGCGCAAAAAGTGCACATCGCGGGCCAGCCGTCGCCTCGGGGTTTCAGGCGGCGC
CCGGGGCAAAAAGGAGCTGAGGATTGGTTTGGGTCAAAACACCCATTTCAGTTGGCAGTGGGGT
CCTCTGCGCCTACAGGGCTTCGAGCTCCGGGTGCGGGCTCACTGGGGCACCATGTGCCAGCGACT
CGCCACCCGGAAATACTTGGGTTGCGCTAGAATACGCAATGGGTCTGTGTTCG
CGI: 392 range = chr9: 101621613-101627382
CGGGCCGCCACCGCCGCCTCGGCTCCTCCGTGACGTCCGGGAAGGGTATCCCCGGCACTCAGGAA
GTGGTGGCCGCGGGCAGCTCCGCCCCCACGCGACTCGGGGCTGGCCGAGCTGCCGGGTGGTAGA
ACCGCCGGGCCTTCCGCAGGGCGGGCGGGGATCACCGTCTCCCCACCTCCGTGCGCACACACATG
CACGGGCCTCGCGCCCCGCTGTCGCCCGCGCCTCGCCCTGCTCACTTCTCACAGCCCGCGCCGGA
CGAAGAGAGCCGCCAGCGCCCCCCCTCTCTCCCACGGCTCGCACCCACTCACCCCGACCCCCGGC
GCCGAGCCCGGCCCCTCGTCGCCGGTCACCAGACCTGGTCGAACCGAGTCCAAGATGGCGACTCC
GCCCCCGCCCCCTCCGCACTCCCGGCGGGGTCAGGGCGCCCCCTCACGCGGCGGGCTCGCGCCCG
AGACCCGGCTAAAAATAGCCAGGCCCGACTATATTTGGTTCGGCCGGATCCTGGCGCGGCCGCGC
GCTCCCCGCCCCGCCCCGCCCCGCCCCGCGCGCCCTGCCCCGCCCCGCGCGGCGTTGAATGGAGA
AGGGGCGGGGGTGACCGAGGGGAACCTACTCCGCTATCTGCGGCGCGCGCCGGCGGCGGGTCCC
GGCCAACCGCCGAATTTAGTAACATCGCCTGCGTCAATCACGCGCCTCGCGTGCGTCAGCGCCGC
GCGGCTCCAGGTCCTGCTCCCCCCCTTCAAGCCTTTGAATGGATACAATGTAGCAGCGCCCTCCTT
CCTTCCGAGGCTGGATTGGAACCCGCCGCAGTGCAGAGACTCGGTTGCTCTCGGCTGGGTCAACT
TTCGGGGCATTCTCCCACGATCCTCTCCGCACCACCGTGTCTGAATTGGAAGTGGAGGCGAAGAA
AGATATACATGCCATATTTACCTATATGTAGTTTGTTTTCAAGTTTCTGGTCCTAGCTCGAACCTTC
TTCGATTCTGAAATGTGTGCTGTCTACAAAGGAATCTTGTATCTCCCCTCGGCGCAGCGCCCCCCG
CCCCCGCCACACACACACAAATTGGGACAGGTCAAACATATAAAACGGTATTTGTGATTCAAGCG
GACCACATGGGGACCACTCTATCTGCATTGTTTCACTCAAATATTTTCTCCTGTCCAAAAATTCAT
TTCTGAAAGAGACTGCGTTCACTCAGCAGCAACCTTTGGGACTAGGGGTCTTTAACTCTGATAAA
TTTTGTTTTCATCAAGAAATTTACACTTAAATTTATCATTTCCAGGAAGAAATTGCTCTCCTTCATA
CAGTCACCCAGGCTTTCGGCACACCATTTCATGACAAATGTGTCCGAGGAGACCAAAGCAAATCC
CCTAGCGAGGGACTGACTAATAAGTCCTGTTGATTGATTTCGAAATGTTTAATTTGGGAGATGTG
GGCGGAGGGCATCTACAACCATCAAAAAGTGAAAGTGCTAGTTGAGAGTTCCATTTCTGACCCGG
TGCCGGGGAGGAGGAATGATTTGCAATAGTCAGACCCGCTCAGCTGTTCAACACGTGTGTGTTTG
TTTTACACACAGAGTAGTTTCTGCTGCAGCGCGCGTGTGCATGATGGATGTGCACTTCGCTGGGTT
ATAACGTGTCCAGTTAAGAAACCCACGCCGTACGTGTAAAGAAATCAAACCTTATCCCCGGAACC
ATCTGCATCCCTGTGTGAAACACGCACCCAGTAAATGATGCGGGGAGGGGGGATTAGCCTGGGC
GCAGAGGACCGGAGCAACGTAAACAGCTTTAGAACCTATGCAAGAGGAAAGTGCAGCTGCACCT
CAGGGCGTCTTCGGGCTGGTGCCAGACGCCTTCTGCACCGGCTGCCAGGTCACTGGAGCTGGTCA
GAAGCTGGCTGGCGGAGCCTTCCCTTTCGGAAGAGCTGTCCTCTCCCTTACCCCCCTCGCCCTGGC
TCCGTGCCTCGGGGCAGCCTCGGAGGCGCGCCAGCAGCACTCCTCCAACTCTACTCCACCCGAGC
CTGACAGCTGGGCGGTCCCGCCTGACCCGTGGGCAGGCCGCTGCACCCTCCCGCAGACGCACGCC
CTGGCGAGCGGTTCCGCTGCAAAAAGAGAAGCCCCCAGGCCGGGGCCGGCCGTGCGGCGGAGTT
TCCATTGTGCGGCCGTGCGACTGGCCGAGGAACGCGCGCGCGCGCGCGCGCACACGAACACACA
CACCCTCCCTCGCACACGCGGAACCGGCTGGGCCAGGGGAGGGAGGAGGAGGGTGACGTAGCGT
CCCATGGCGTCACATTGACGTCTCGCATTCCAGGCACTCTATGGAGAGGCCGCTAGGGCTCCTGT
GGCATAAATGACGTGCCGAGAGAGCGAGCGAACGCGCAGCCGGGAGAGCGGAGTCTCCTGCCTC
CCGCCCCCCACCCCTCCAGCTCCTGCTCCTCCTCCGCTCCCCATACACAGACGCGCTCACACCCGC
TCCCTCACTCGCACACACAGACACAAGCGCGCACACAGGCTCCGCACACACACTTCGCTCTCCCG
CGCGCTCACACCCCTCTTGCCCTGAGCCCTTGCCGGTGCAGCGCGGCGCCGCAGCTGGACGCCCC
TCCCGGGCTCACTTTGCAACGCTGACGGTGCCGGCAGTGGCCGTGGAGGTGGGAACAGCGGCGG
CATCCTCCCCCCTGGTCACAGCCCAAGCCAGGACGCCCGCGGAACCTCTCGGCTGTGCTCTCCCA
TGAGTCGGGATCGCAGCATCCCCCACCAGCCGCTCACCGCCTCCGGGAGCCGCTGGGCTTGTACA
CCGCAGCCCTTCCGGGACAGCAGCTGTGACTCCCCCCCAGTGCAGATTTCGGGACAGCTCTCTAG
AAACTCGCTCTAAAGACGGAACCGCCACAGCACTCAAGTACGTATGAGATTCGCCCAGTTAATAA
GGGGACTTGCTAAAAAAGTTGGCTTGCTGGGAAGCGTCGCTCCTGAAATTGACAACTTTGAGTGT
GGGGTTCCCTCCCCTGACCTTGCCGCCCGGCTCCGGCTTTGCTAGCCCAGCGGCCCGTAGGTTCTG
ATCTGAAACTTTTCCCCTGTAGTGGGCCCGCGGGGATCTCTGGAGCTCGTGGGATTCCTCCCCCCA
CTCGAAGAGGCGAAAAGCCTCTAACAAAGAGGAGGACCGGGATTTGTGCTATAGCGGCTCCAGC
GATGTAATTCAGGGTATTTCGGCTCTAGTTGTCATGGTAATGATGCTCTCGGCGGCGGCGGCGGC
GGCGGCAGCGGCAGCGGCAGGGAGTTGCAGCTCCGGTGATGAACGGCAGTAATTTTCCTGCCTTT
TAAGTAGGATTGAAAATAGGAGCTCTGGTGGGTCCAAGTAAATGTTGCTAATGGTGGCACCGAG
CTGGTTCTCTGGAAGGAAGCTTAGGAGGGAAGGGCCTAGGCAGCGACGGCCAGAGTTTGCAGAC
GCACTCGGAGGACCTCAGGAAAGAGGGCGTAATTGTAGGAATGTGGCTTATTGTATTGAAATGA
GCCTGGGGTTCCACTAGGCACGTCTGCCTGTCGTCGCTGACATGCCGGTTTACACTTTTCCCTTAG
GAGGTAAATCGGGTGTAATTGGCGACTCCCGCACACTGACACGTGTGGGGACGGTGTCCCTCTCC
TCTGGACGTTGGCTCGGTGTGGGAAAGGCATGCTTTTTCACGGACAGAACTCGCGCTTTTGGAGA
AGTTGCTCCGAGTGTTTTACTCTTAGTAGAAATGAAAGTTCTCGGTGGTGAGACGAGAGCGGTCA
TGCGAGCGCAGCCTGCGGAGACTGACTTCCAAAGGCACCTCTCCAACCTGTTGGAAGCCTCGGGG
CAGTGGTGAGGAAGAACGCATTTGTCCCTTGATTTCCAAAAACGTTCTTGGAGATTGCCTTTTATT
TATTTATGCGTCCGGATTCTCCAGAGGGAAGAGCTGCGAGGAGTTAGTAATCAGAAATGTCGGGC
TGTGATCCCAGGGCCTTGTGGTGCTGGGAGAGAAGAACAGGGAGCCGTCATTCCATCGCGAGTCC
AGTGCCGGATCTCCTTTTCCCACGACCGGGAAATATTTTATAGAATCAAGTTAGGGAAAGAAAAA
GCTGCCTAACGTGGCTGATGGGCACAATGAATGAAGTTACTTTATTCGAGTTATTTTCGCTGAAC
AAGATGCTCGCAGAGTTAGTATCAAAGCCTCCTTAGGCCACAGCAACCTCCACGTCCGCTCCGCC
CCCCACCCCCCGGCACCCATCCACCCCTTTGCCTGGGACCGTGCGCTCACAGAAATTCTTCCCCGC
GGTTTGTCTAGTCTCCCTCGGTCTTTCTGAGAATCTCAGTCTTTCCCTTTCTGTCAGTCTCTCCCTC
GCTCCTCTTGCCCCCGCCCAACCTCCGCCCCCGCTCCCCAGGGTTGGAAACTCCGCAGAGGAGCC
CCGGTCACAATGGTGCGTTTCACGGTTTCCTCCACACACTTCACCACCGGGAGGGGAACACGGGG
GTGTTCCTTTCGTCTGCCCACGAGAAAGAAAGGGGCTTTGACAGAGGTAGTTATTTCTTCCTAATT
TACAACCCCAAGGCTCTGTGCTCCCGGCCCCGGCGGCCGCAAGCAGGAGCGGCGGAACGTGTTTC
CAGGCACTGAGGCTTTGCCTAGGTAACCGGCCGCTTGTGGGGTGGAGAGAGCGGCGGGGCGCTG
GGCTGGGCTGGAGGAAAGGCTGTGTGGGTCCCTCCCGCGAACGCTGGGCGCTCGAGGGGAACTC
CTTCGTTGGGGAGAGGCGGTCCCCGCCCACCCGAGTTCTGAGGCCTCCTCTCAGAGAACGACTCC
GCTTGAAAGGCCCTCAGGGAATGCGGGACGGGAATGCGGGCACCCACTGAAGGGGCGGGTGGG
GGATTGCTGGAGACCCCCATTGAAGATGCGGGGAGACGGGAAGCACGTGCTACAGCCGTGGGAA
GATCCGCTTCTACCCGCGCTACATCGAGCGCAAGCTTTCCGTCCGAGCAGCTCCAGCTGGGGCTG
GGGGTGGCAAGGGAATCCCCCATTTCCTGGTCGTCGAGGGTTTCCTGGACCTGGGAGCCCCGAGT
CCCTTTCTCTCTTCCTGCAGGCAAGCCCCGGGCTCAGGGGCGCCAGGAGATGACCCTCGTGGCCC
CGCGCACCTCGGAGGTTTTTGGTTTGGTTTGATTTGGGGTTTTTCGGTCCCTGTTCCCACTCTTCGA
GCTGCGCCCACCCCGGGCAAAGGGGGCGCTATAGGCCGGAGTTTGGGCACCCAGTTCCTTCCCTG
AGGCCTGCTGAGCTGCGCTTTCAGCTACAAAGTTTCGCTGAGGCTGTGGGCTGAGACGCTGGTCC
CGGAGCTGCGCTCGGCGCCCTCAGGAATGTGCCCCCGCTAGGCCGCTGGGTGGCCAGGAGCCTCC
AAGCCGCCCACCCTCGAAGACACCGCCCTCTGGGTGCAGGGGACCTGCCTCCGCTCGTCCCATCA
GCCGCTAACGCCGTCCGCTTCGTCCCCTTGCTTCCAGCGCCAGCCTTCTCATCGCTGTGCCCTTTT
GTTTGCTTGACCCCTGGCCCTCGAAACTCGCGGCTAATAGAAGCGAAGCTCCATTAGCATTTAGA
ATGAAAAGCGCAGACTTTCTTAATTCCTCGGGGCATTCATGCATTCGTTCCGGACCTTGTGCATTT
CCTATGCAACGTGTAAAATTTGTATTTGAGGGGTGGGGGGGGGGGGAGCTGGAAAATGGCTTTTT
TCCGCTGGTGAACACTCACTGACCCCCCGTATTCGGGCGGAGCTCG
CGI: 56 range = chr10: 13556172-13556753
CGCTGTTAGATTTCGGGTCAATATCCATCATCGCTGTTAGACTCGGGGCCGACATCCGTCATCGCT
GTTAGACTCGGGGTCGACACCCATCATCGCCGTTGGACTCGGGGCTGATACCCGTCATCGCTGTT
AGATGCGGGGTCGATATCCATCATCGCTGTTAAATTTGGGGTCGATATCCGTCATGACTGTTAGA
TTTGGGGTCGATATTCCTCATGACTGTTAGACTCGGGGCTGATATCTGTCATGACTGTTAGACTCG
GGGCCGATATCTGTCATCACTGTTAGACTCGGGTCAATACCCGTCATCACTGTTACACTCGGGGC
CGATACCCGTCACCGCTGTTGGACTCGGGTCGATACCCGTCACCGCTGTTGGACTCGGGTTGATA
CCCGTCACCGCTGTTGGACTCGGGTCGATACCCGTCACCGCTGTTGGACTCGGGTCGATACCCGT
CATCGCTGTTGGACTCGGGTCGATACCCGTCATCGCTGTTGGACTCCGGTCGATATCCGTCATCAG
TGTTGGGCTCAGGTCGATACCCGTCATCAGTGTTGGACTCGGGTCGATACCCATCATCG
CGI: 22 range = chr10: 15294605-15294858
CGTTAGAGCATAGCGACATCACGTGCGGTTTCTTAATGTCCCTGGTGGCGGATACGCCGAGTCCT
CGGAAGGACATCTGGACACCACTTTCAGCCACCTCCTTGCAGGGGCGACATCCGCCAAAGTCATC
CTTTATTCCGAGTAATAACTTTAATTCCTTTCTAACATTTACACGGCAAACAGGAATGCAGTAAAC
GTCCACGTCCGTCCCACGGCTGGGCTGCCGTTCCGTTTCCTCCACGAACGGGTACGCG
CGI: 987 range = chr10: 76825135-76839606
CGCTCCCAAAGCCACGCGAAAGTGGAAGCGCGGGGCCTCGGGATGCCCATCCAGCCGGCTGCCC
GCGCGCTGGCCGGGGTTCCAGGACTCCTTCCTGCTCCGCCGGGCTCTGCAGCGCCGCCTGGTGGA
CACGCGGCGCTCCGCCCGGGCTCCAGCCGCCAGCCGGCGCGCCGGGGATCCCGCGGCTGTTCGG
GAAAAAGCTGGAGAGAGTCGGGGGGCGCGCCTTGGGGAGGGGCATTTGTCCCTCCGAGGAGGCT
CGCACCTCGGCCTGGACACGCAGCAGATACTGGCACCCACCCTTCACTTACCGCGCGCCCGGGGT
GACTCGGATCCGTCCAACACGTCGGGGAATCCTTTCTGTCCTCACCCCCGGGCGCCCCAGCGCCG
GAACGCTGCTGCCTCTGTGTAGCTGCTCCCGGAAGGAGTTTCATCAAACTTTTAAGGGGCTTTGGT
TTTGGGTTGTGTTGATAAAATACCAAGAAGGGGCATGAAGGCACGAACGTCCCGGGTTCGTCTCC
CTGCTGGTCCCAAGCCTGAATCCCAGGGCGGGGGAATGTCTAGGTCCTTCCGGGCCGGCAAGGTG
TGGTCGCTCAGGGACCCCTGTCCGAAGACGCTAGGGAAAAGAGGCACAGCCTGTGGGTCGCAGT
GGCCAGGAGCGCGTGGCCGCTGCTGGTGAGAGAGTGGTAGACGCCTGGCTTTCAGGTCCTGAGCT
GCGGGCACCGGAGCGTGGGACCCCGGCTGCAGCACCGCCACGCGCTCCCGCCCTCCCACCTGCA
GGGCGGGGGATGTCTGTCCAAGAGGCCGGGGCGACAAGCCCGCCGGCCAGGATTCTCAAGGAAC
CAGGCCCAGCTCAGCCTCTCTCGGCGGGACCAGAGTGGGACCGGGGCCGCGGCGTCCGAAGACG
CTGCGGGCCAGGGGTCCTCCTCGGCGCCAGCTCCGTTTCCTGGGGTCTCGCGACGTCCGGACATC
AGGGTCGGGGGTGTGGAGACGGCGGCGGAGCCAGAGTCCCCACCAAGTCAGTTCCAGGAGGCGG
CCCGCGCGCTTCCCGCAGTGTCCGGGAGGTCGCTGGGGGTGGCTTTGCGTGCAACCCGGGTAAAG
GCCCTGCAGCCGTGAGGCTGGCGCTGGGAGGAGGGTGGAAAATCTCAAAGTCACCAATCCCGGT
GCAAATGGCGGCAGGGGCCGCGGGCTGTCGGACGCAGGCGAGAGGCCAAAGGCTGACTTCGCGC
GGCCGTGAGTCCCCAGAGGCAACAGGGGTACCTGAGCGCCGAGGGGATCCCGAGACTCGGAGAA
ACCGGAAGAGCCTGACCCCAGGGAGCGGAGAGTTTGGGGTGCGCTCTCAGAGCTGTGACTCCAC
GGTCCCGGAATCCTTGGAAAGGGCGCTCTGGGCTCAGAGCTCCCAACTAGCCGGAGGACCTGGG
CTAGCGCCCAGGCCTGGAGCGTCTGGGAGGGGGCGCTGGGTCTCGGCCCCCCTCCCCCCAAAAG
GGACTGAGACTTTTTTCTGCGTGCTTCCTTCGGCGCTTCGGGCAGCTCTGTCCTGCGGCCCAAGCT
GGGGAGAAGACAGCGGCCCGCGCCACAGGGAGCTGCGCCCGGACCCAGACTCCCGCCGCGCTTC
TGCAGAGCGGAGCCCTAGGTGCCCACCTGGTAGCCCCAGAAAGGCCGGACCTGGGCGCCGGGAC
GCTCGCGGGGCCGCACTTGGAGGGGCTTTCCGGGTCCTGGCCGGGCGGGCTCTCCTGCGGCGCGG
AATGGAATAGAGCGCCGGCTGCAGAGCCACCCGGACGGGGAAAAGCAGCGGTGGCGCCGGCCA
GCCCCGGGTCCCGACTCTGGAGGGAGGAAGGAGCGGGCGGGTGGGGGTGGGGGTGAGGGCGAG
GGTTGCGGGGAGCGTTTAGAAGGCCCTGGGCAGCCAGAAGAAGAAAAGAGGACGCACTCTCCCC
TAGGGACCAGAGGGTCCCTGCGTACTCCCCCCAGGCCCGGGACACAGGTTCCCCCAGCGCCCCTC
CGCCTCCCAGTCTATTCGGCTTGCCCCAGCGCGCTGCCCCACGTCCCCGAGGCCCCGGCCCAGGC
CCAGCCGGCGAGTCCCGGCCTGCTCCACTCTGCACAAAACGAAACCCAAATGCCCAAAAAGCTC
AGGAGGGAAATTTAACAAAAACCCTGTCCCCCGCCCCACACCCCCTTTCACTTTTAACAAGCCAG
CTGCCAAGAGAAAATTGAAATAAAAACGAAATGATAGATAGCGGAGGACACTATTTTCCAAATG
GTGAAATATCCTCTAAAAATATGTTCCCCAAGGCCAACTTCGCGGCTGGTAGCCCCTTCCGACGC
CTTTGCCTCCCAGAAAATCACAACAAAGCGATCGGAAATTCAGCCACGGTCCCGGGAAGAAGGA
GTAGCAGTGAGGCCCCGGAACCCACTGCGGCCGAAACTGCCATGCTCTCTTTAACCAAAATAAAA
AAGATAAGAAGAAGAAGTAAAACCCTTTAATACATCAAATATACGGAATTTTAATCTTTAAAGCG
ATACATTGTCTATTATTTTAGTACATGACGTAAACCTTGTCCCCTTCTCAGCGGGTGGACTTAAAA
ATTAAAAATAGTTAAGTGTTCCTTTTAAAGAACAAAATAAGGCAAATGAGGTTTTGGAATAGAAT
TTTTTCTTTTTCTTTTTTTTTTTGTTGTTTTCTTCCAGAATACATACAAAAAAATACCCATTCTCTTC
GATGGTATACACCTTAAAAATAATTGCAATTTGAAATCAGAGCTGACAAATTGTGACTTTTTTTTT
CATTTTTTTTGTAACAAACATGCATGTAAATTTGTGTTTCAATCAGACATTAAATAACGTACAATA
CAATCATAGCAATTTTAAAGTAACATTAAAGAGAAGACCTCTAGTTCTGTGGCGGTTTTGCTTTTA
TTTATAATCTACAAGGGATGGGAGGGTTTGTTTTCCACATTTTTACCCTCAAGTTTTAAAATTTTTC
CCACCTCCTTTTTACCTACAGAGCTCAAACTAAATAATGCACTTTTGAACCACCGGTCCGCTTGGA
GCTAACATAAATAAATACCAGTGTCCACCGATGCAGTCCTCAGATGAACACTTTCTCAATTATTTT
TTCCTTTTTTTTTCTATAAAAAGTCAAATGATATTTTTTCAACTTTTATAAAGTTTGGGTGGGGAGG
TGAAAGTGGGGAGAAGGGGAGTGTCCCACCGGGTCTCGGTCGCTGCTGTCGGGTAGATAGATAC
GGTATACATTTCTTTCCTTTCGTGGCCCGAGTCCTCCCCACGCGCGGGTGTCAGCAGCCTGGGCCG
TGATCCCGCCTCTCCCTGGACTCCTCCCCTCCCCCTCTCCCTCCTCTCCCTCGCTCGCCCTCCCGGC
CGCCCTCACTGATACCCCAGCGCCGAGGCGGTGGTCAGTCTCTGTCCGTAGAGGGCGTAGGGGGA
GTAGTACGGTCCGGTGGCGGCGGGCACCGGCACGGGGGCGCCAGGCGTGGGAAGCGGGCTCTTG
GAGTAGGGGTGGTAGCGGCTGCTGAGTCCCAGCGCGTGGTGGGGGCTGCGCAGCGCCAGCGTCC
CAGGGCTGCCCGGTGCGCCCGAGGTGGGGATGTGCATGTGGCAAGCCATGGCGGCCGCGGCAGC
GCTGGCCAGAGACGACGAGCTGGGGTAGCCCGACAGCAGTTTGTCTGTCCCGGGAAATGCCGTA
TGGGTCCGCAAGTGGCTCAGCAGCTCTTCGGACGTGGCGAAGCGCTTGTCGCACGGCCCGTTGGC
CGACACCCAGTTGCAGATGTGGGGGAGTGGGTCGTTAGGGAGCATAAAGCCGTAGGGGTAGAGG
GGGTGGCCGGCCAGGGAGGGCGGTGTGGCGCCAGCAGCCGCGGCGGCCGTTAGCGAGGAGTGCA
CACCGTGCAGCGGGTGCGTGGGGTACACCAGCGGGTATCCGGACTTCAGCGCCGCAGCCGCAGC
AGCCGGATCATGTGCGCAAGAAGCGCTGGCGGCCGCCGCCCCTGCCAGGTGGCTAGCGCAGTGG
TAGCTGAGGCAGTAAGGGTCCCGGCACAAACTGGCTGTCATCACGGACGGCGGAGACGCTCCGG
CCAAAGGGCTGGAGCCGGCCGGCTTACTGCAGCCCAGAGACCCGGCCGCGGCCGCCGCCAGCTG
CGCCCCCACCAGGCTGCCCGGCTTGGTGGGGTCAAGTGCCACGCCGTGTGGCAGGAACTGGGGC
GGGTAGCCGGCGTAGGCCCCGGCCAGGCTGCCTGGGTAGGTCATACCCGCGGGAGGCAGAGGGA
ACACTGTCTGGCCCGGCTTGTAGGGTGACACGGGAGCCACCAGCCCAGAGCCCAACACTGAGGA
GGAGGTGGGCGCGCTGGGGCCGGAGCCGGAGCTGGAGCCCGATGAACCGCCGCAGTCCGAGCCC
AGAGCCTTGCCTCCCGGGCCCCCATCCGGATGCTGGTTCACATCCACATTAATCCCGCCGCCGCA
GCTAATCCGGCCGTGTGCCAGCCCCGTGGGTCCCCCTTCGGCCGAGGCGCCCCCGGTGCCCTTGC
CACCGCCGCCCACGTCGGTGTCTTTCTTGTCGTCCTTGCCCTCCGGGGCACCCCCGGCCGAGGACA
GCATACCTCCCGGCGAGCAGGCCGAGGCGCTGGAGCTCGGGCTGCCTGTCCTGGGCGTGAATGG
CTGGCAGGTGGCGCTCGGTACCCGGAATCCCGACTTCTCCGACGAAACACCCCCGCCGCCGCCCC
CGCCACCGCCACCGCCTCCACCGCCGCCTCCCGGCTCCTTCTTATCCGAGCCGGGTTTGGAGTACG
GCTTGAAACTCGACTTGTCCTCCACGCCGATGTCGCTCAGCTTCAGGGGGCCCGATTTGGTGTCCT
TGTCGCCCGCAGCACCGCCGCCGGCACCGCCCGCGCCGCCCCCGTTGGAGGCAACCGAGGAGAG
TTTGGAGGAGGGCGAGGGGTCGGGCTTCCCGATCTGCGAACATGTTTGCGCCAACAGCGCCAGC
GGGCTCTTCTTGGCATCGAGCTGCGGGGTCAGACGATGGGGGGGGAGCGTCACACAGAGAAAGA
AGTGGAAACCCTTTAGAATCCTGGCTTCTGGGGTTCAAATGCCTCTCCGCAACAGCCCACGAAGA
TCCTCCCAGCCCCAAGGCGCAATTCTAGGCGGCACCCCCTCTTCAACACCCACTCACAAACATTC
TCGCCGTTGGGAAAGGCAGCAGATTGCTCCCCCCGCCCAACCACCACCCCCAGCAGCATCTTTCC
TGCCCTCTCTGAGCGCTAACTAGATTTTTAAAGAGCAAAATGTTTTGGTTTTCGGTTCCTAGACCC
AGAACTGTACCCCCTCCCCACAAACACTCCTTAATTCTTCAGAGTAAGGGCTCGGGCGGCTGGCG
CCTCAGAGGCTGTGTGCACACACTGGCTTCTGCGGATACGAAGGACGGCGACTTTGGAACCGTAT
TTCAGACCTCCGCCTGCCTTCGGTGCCGAGTGAAGGGGCCTGGGTCGGGGTAGGGGCTTTCATAT
CGAAAGGAGAAACAAGTATTGGCAGCCTTGCTCCCTAGCATGCACGCCCCATTCCACTTCCTCCC
CCTTTTCCGCCCTAGTTTTGAGGTACGGAAGCAGTAGCCTCTTCCCCCATTCGGGAGCTGAATCAC
TCCGACCCCCCCACCTCCGCCCAGATCCCGAGAAAAAGAAAGCCCTAGGGTGGCAGCCAGGGTA
GTGGTCCCAGTGCGATCCAGAGAGAGGGTCCTTACCTCGATGGGGCTGACCGGCGTGGAAGGCA
GGGGCTGCAGGTACTCGGGGTGCAAAATGTGGCCAGTTCGTGCCGTCAGCATCTTCAGCACCTTG
ATTGGCAGGCGGTTGGCCTGGCGCAGGGGGTCAGAGGGGGGCACGGCGTGCACAAAAGGCTTGG
TGCTGCCGGCCGGGGACGAGCCTGGGCCGGGGCCGGAGCTATTTCCAGAGAGCGCGCTGGTCCA
GGCAGGGTCTGCACCGCCGCCTCCGCCTCCGCCGCCGCCGCCGCCGCTGTGCTTACTGCTTCTTAG
GGCAGAAAGCGAGGGCGCTGTGCTCATGACCCACCCGCGCGCATGGGAGCAGCGGGGGGGAGG
GCTCCGGGAGGCGCGGGGCGGGCTCGGGGCTGCGCGCTCGCCCCGGGAGCAGGAGCAGCGGGA
GGAGGAGGAGCTGGCGCGGCGGCCACGGGCGCCCAGCGCGCCTTCTCGGCGCCTGGAGCCAGAC
GCGAGTAATCCTGGGTGGCCCGCAGCGGAGCCGTGGCCGGGCTAGAGGAGCCGGCTGGACTGCG
GGAGTGCCGGGCGGCTCGCGGTGTCCGCCTCGGGCTGCTCCCCTGCGCTGCGTTCTCGCGGCCCC
GCGCCGGCGCTGCGCTCTGCGATGCGAGCCGCTGCGTGTCCAGCCGGGGCTCTGGCGAGGAAACT
CACTTCAAAAGCAGCTGCACCAAGGGGGAGATTAAAGCCTTTGCCGCCTTCCAATCAAATGGGA
GCCCGAGGTGATTGGAGGGTCAAGGGGATGTGACGCGTCCCGCGCCGCCGCCGCCGCCGCTGCC
AGGACTCTCAGTGCCGGTTTTAATGGGCAGCTCCCTCTTCGCCGCCCCCCTGTGTGTCCCCTCCCT
CGATTTCGCTGGGAGGACGAGATGGACATTTATTCTACGTTTGCCATCCGCCGCCTCCCTCTTTTT
TCCTCCTCGTCGTTCTTCCTTTCCCCAGCTCGAAAATAAACTGAAACCTTCTTTTGAAGGGGGGTG
CGCGTGAGGAACAGACTGCGGGGGGTGTCCAGAAGGAGCACCGGTGGGAGTGTGGACACCGGCC
GGACTGTCAACTCCAGGGGCGAAGGGAACCTGCACACCCAGTGTTTTTTCCTTCGCAGTAATCGA
GATCCCGCGCGGCGCAGCGCAGCCACCAGGGTAAGAAGGCAAGGTGGGGAGCCGGAGCTGGAA
GAAGCCCGCCCGCCCGCTCTAATTTCCTCAGATTCCGCGGCGGAGAAACCAGAAGCTAGATGGGC
AGTCGCAGCGGCGGCGGCTCAACACCGCGAGGAGCGCTGGGCTCTCCGCCCTTCCCGGCCACGTG
ACGCCCGGGGACGCGTAGATTGGGGCAGCAGCGGGGGTCACATGTTTCCTCTGTTTCACCCTCAG
TCTGTCCCCCAACCCCCCATTCTTACTCTCCCACCCTGTTTTCCTCCCTCCCCCCCTTCTTTGGGCA
TCTCCACCCCTCCATCAATTGTCAATGTTCCTCGACCGCAATCAATCAGTTATTTGTCAGCTCTTGT
CAATCCTCCCGTGATTTATGTCAGCTTTTGTTGCTGATTACAAGGCGGGTGCGACTTGAAGGGAA
AAAGAGAGAGGGAGAGAGAGACGGAGAGGAAGGAGGAGATTGAGAGGGAACTGGAGGAGGGG
AAAAGAGGAGCGGCCTCCTGGGATGGGGGTGGGGTGGGGGCTCTAAGAAAAAGAATGAAAGAG
GCGCACGGTGTCAGGAAAATGAATAGCGAGAGTAAAGTGCGCAGGTGCGCCCAGGGCGCCGAG
AGGGGCGCGCAGGCCTGGAGTGTGCGCCTGCCCTCTCGGTGTCGGAGAGACGCCCTTCCACCTCT
GGGAGCCTCGGTCTGTTGGGGTCGCGGAGTTCGGGCGCGGCTCCGGGTACCCGAGACCAGCGGC
GGCAACTTCTAACACGGGAGATTTCCCGCCACCCCACCCCGCCGCCGCGAGTCCTCGCGGGGCGT
GTTGCGTGCGGAGGTCAGGCTGCCACCCTCTGTAGTTCCCTAACCCCAAACTCGGAGACTTCTAA
GAGCCACACCACCAAGGAACTTCTAGTCCTGGAGGTCAATGGTGGGGCAAACCTCGCCTCAATTC
TTTGACCCCCTCGGGTCGTAAGCAGGGCTAAGAGGCTGCGAAGAAGAGGCCTGGCCATGGGTGT
ATGGGGGAAGAAACATCTCAGGCTCACTCATGCCCCCTCCCCGACCTTCTCCCACCTGCCGCCAT
CCCCGCAGGCTGGGGAGCCAGGGTAACTCGGGGCTGCTCTCTCGAATTTATTGGAACGCCGAGTC
GGAATGAGCTGCGCTAGGAGAGCCGAGGGAAGGAGCGAGAGAGGGAGGGGGCGGCTGCCTGTG
GGAACGCGGGTTCTTCCAGGGAAGCGGAGCGGGACTGCCGCGTTCCCCTCGATTTGCACCGTCAC
TCGGGTTGTTTGGGAAGAAAAGGGGAGGCGCTGTGCGTGCCACCGGGTACCTGGACCTGGATGC
CCAGTGTGTATGCATGCCTGTGGCGCTGCGTGAGTTATCGGGGCTCTGATAGCGTCCGGAGCGGG
TTCGAGGGTCTCCTCCAGGAACTCGCAGAAATTAGAGGGGGTGGGGAGGAGGACACACCCCCTT
CCTCGGAACGACTTAGAAGACTTTGAATCTCCCCTCCTCCGCTTTTCCGCCCCGGCTCTTCTTTTGT
TGTCAAGTCCTTTTGATAAATGGTGGGGCTGGGAGCTCTGGGAGTCGGGAGCCAGTCTGTGCCCG
CGTGGGGGGGGGGGGCCGAGCCTGGAGACCCGGTTTCGACCGGCGCGCACCTGGGGCTGAGCCG
GGTGCGCGCGGGGGTCGGGTCTGGAACTGCCCTTGGTATGCTCGCCCTCTCCTCTTGGCCCATCTC
ACTCCCCTCCCCCACCTGACTCCCCTCCCCGGCTTCTTTCTCTGTCTCCCACTCCGCACGGGTACCG
AGAACTTTCCGGGGTGAGTTTTAGAATTTGCTCCTAGGCATTCTTTTTCTGCGTTTGGATTTTATCC
CTTGATTCTTTGGGTTGGTAAACTTTATGAAATGGTAACTTGACTGAAGCTCATTTGGAGAGAAG
AGGGGAGTGAGGCGGTGTGTGCTTGGGGCTGGTGTTTGTCAGTGTGAGCGTGTAAGAGCACACCT
GCATTCAAGGGGGTTTGTAGTGTCCGTGCGACATCTGAGGGAGGTGAGTCGGCGAGCGGGTGGC
GAGGCCCCACGCTGAGGGAGGCGGTTGTCTGGTCTCCGGGGAGCAAGTCCCAGGTGCGCGTTTCG
GAGGGCGGGACAGTCCTACGCTGTCTCTGCGGGGCAGCGCGTCTGTGAAGCACTCTTATCCTTCG
GGTGTGTCCATGTGTGCCTGGTTACTGAGTGCGCGGCCGTCTAGGTGTCGACCCAACCAATGGTA
GTGCTCCTAACGCCGCGAGCCCACCTTTGGGCCGCTATCGCCGCGGCCTCCCCGCGAGGCCCGGG
CACTGAAATTCTGGGGCCTGCGACAGGGCCGGGGGGGACGCAGCCAAGGGCGTCCCTCCCCAGC
CTCCAGACTCAGCTCTTCCCCCTCTCCTCTAATTCCAGCAGCTTATTACGCCGGCGGCTCAGGGGA
GGCAGCCTCAGCACCTGAAGCCTGGGGGGGCCGTGGAGTGGCTCCTGCGTCCCCCGGTGTGGCCA
CACACGCTCGTGGGGCTGCGCTGTGCTGCGTGGGTGCGGGTCGTGGTCAGCGTGCGTTCGCTTTT
CTCAAAACTCCACTCCGAGGGTCCGAGCGCATGGGGGCGCCTGGGGGCCTGCGCGCCCGGGGCT
TTTGGGAGGCGCCAGCGTCCGAGCCTGCGCACCTCGCGCAGGAGGCCAAACCCCCGAAGGCCGG
CGCGGGCCCGGGAGTGGGGGCCATTAATTACTCTGCGCCAGGCCTAAAGCCTCCAACCCCCAGCA
GCAGTTGGCGTGGATGTCCTGCGGATTTTATTTGCAAACAATGGAAATGATTTGTTTTCTCTAAAA
AAGGAGTGGGCAAGGCAGATATTGGAGGAGGGGGTGGGAGGAGGAGAAGGAAGGGGAAAGAG
CTGAGGAAAAAAGTTTGAAAATGCCTTGAACTATTCACTGTCGAGATGGCTAATCAGTATTGAGG
CCGGAGGGCAGGCGGGCCGCCTTTCACCGCCGCTTCCCTTTCATCTCGGGCTCGGCGGAGGCGCT
CAATTAAAAGCCTATCAGTTTGTAAGTAAATCAACGCCTATCAGAGTTGTCACATCAAACAAAAC
GATTATTATTGCAGCCCGCCTCCCCTATTAATCTCCCTCGAAGATAATCCGCCTGACAGGTCAGGC
CCGGCGAGCTGGAAAGCCTGGCCCAGAGCACCCAAACAGTCCCGGACTGGCGCGCGCCCCCGGG
GGCCTCCCCACCCCTGTCCTGCCCCGCCTCGCGGTCCCTGGGAGCGGGGAGAGAAAGCGCGCGCG
GAGGCCTCTGCACCCAGGTCCGCCCCACCTGACTGGGAGCAAGGCCCAGATTCGCGGGGCCAGT
AGGGGACAGTCCCGCGCGGCCCTCGCCGCTGCTCGGGGCAGGGGAGAAGCCTGGCGTGCAGGAG
CCGCGGCCCGGCCCGGGGTCTCGCCACGGCCTGCGCTCTCCTCGGCTCTCATTTATTTTCTTCCTC
CCTTCTTTTTGTTTTCTCTCCCGCGTCTTTCTGGGCTCCCCCATCTTTTTCCTTCATTTCCTTTTCCTT
TTCCCTTTTTTTTACTTGCCAGGGAGTATTTCCCATTCTGGCTCTCTCTTCTTGGTGTGCCGTATAA
ACCTTTCTTGCGCAGCCACACTGCCTGGAGTAGGATGTTCTCAGGGCCCGGGTTATTTCTCCCGGG
TTGCCCCAGGAGTGTGCAGTTCCCAGAAACTCATGGCAAAGTCCTCTATAATCTCTTTCCGAGCA
GGGGCTCTGAGAAGCTCTGCGCCTGTTTAAGGGTTGGGTGGGTTTGGAGGGGTGAAGAGGAAAA
AGAGGGAGAGAGACTGGTGGACAGTTGTCCTATTCTGGAATTAGTATTTTGCCTTGGAATTTCAT
AGCTGTAACGTTGCCGACCTCGCTGTTGGCGTTGCCCGGGCTTTTCAAATATGTAGATAAGACTC
AAGGGGGCATCCAGGCCTCAGTAGTGACTATGTGTGTGTGGGTGGGTGCCTGATACAAGTAAATA
CACCCAGATAACTGCCTCTGTGGCCTCAAGTTAGTGTCCCAAGAGTTTGGGTGGGGCCTCGCTTCT
AGAAGGCGCAAAAATCGTGGCCTTCTTCCCAGTGGCCTCCGGCTTCCTGCTTTGGGATGCCTCGG
AATCGAGCCGGACCCGCCTTTTTCTGCGCTCTTCCTCCACATCTGTAGGCCCAAACGGCCACTGCC
GGAGCCAGGTGGAGCCAGGATTGAGGGGTAGCTGAAGTGTCTGTGACGAAAGGGCTGCCCGAGG
GAGCCCAGGCCTGCCACCTCATATGTCGGGTTCAGTGCGCCGCGAAGGCTCTCCGCGCCTGTATC
TCGGAGGACCCCCAGAGCGCTGCAGTATTGGGCGTCAGGGCCGAGCCTCTCGCAGCAGTCACGC
CGCAGTCCGAGGCCACGGACATCCAATAGAGCCCACTTCAACTTGCATGGTGCATGGGGTCCAGG
ACCGGTGGCCCCCACACAGGGTTTCCCAAAGCCTCTCCATTTGTCTGCACTCTCCACTGCCGACCT
TGTAGGAAATTTATTTCCATGCCCAGAAATATTGTTTGGACACAGTGGCTCGGTTATCTTCGCGCC
TGCTTTTCTTAAATACATTTATTTCACCTCTGCCACGCTGCAGATATTTAAAGATCTGTAGATACC
GTAACCCTCAGCGATGCGGGACTCACTCTGGTTATAGATAATATTCTCTGGGTGGTTATTTGAACA
TAAGTTCTATCTCCAGCCCAGATGACACCCAAGCAGGGGCTGTAAAGGACACTTGCTCTAGAATG
TTTTCAACTGAAATATATGTCCACACACGGAGAATTTAAGAGTATTTTTATATTTCTCTCTAGATC
TAAATATTCAGATGTGTTAATTACATGCCCTAGAAGCTGGAAGCGATCAGTGGTGTTCACACTGG
ACGTGGAGCTGTTTGTATAATTTTCATCTCCCTGCACTTAAACATGACTCTCAGTCTAATAAATTC
AACCTTGTCATTTTTAGAATCGACGGGATTTCTCTGGCTGTCGTTTGCGCTGCATTTATCCGAATA
CATCCAGCTCGCAGGCATCCTGCAAGAAACGGCTCCCGGCTCGCGTGTACGCCGACACCTCGGCC
CAACGCAGGACTCGAGGTGGTTTCTAGTGCCCGGGTGGCTGCAAGTCTGCCCTCCGAGGGAGGCT
GGACAAGCGGCGCCCCCAGGTCGAGCGGCCTCTCGCTGCCTGGCAGTGCCTGGCAGCCCCCACCT
CTGCCAGTGCTTCGGAAACCCGCCTGGCCAGGTTCGCCCGCGGTGAAAAATGAAAGCAAATTCCC
CAACAGAGGTAGCCGGAACTTTCCTCGACGAAGGCTCCCTCCTGCGCCTGTGTCTGGAGAACCCC
CAGAGCGCTGCAAGTTAGCAAGAGAGATTCGATGGCGGCTCTGGAAGGCGCACGAAGGGTGGGG
CGGGGGAGCAAAGAGGCCACTGGGTGACCCAGCCTGGTCCGGGGTGAAACGCTAGAGAAGGCG
CCTCGCCTTCCTTATTTCAATGCAACTCCTCGGCCCCAGACGGTAAAAATAGTTTCCAAGCTGCCG
CGGACGCCCAGGATGTGTTTGCAGAGATCAAACTGGGGAGGAGGCAGCTTTGTAAAAGTTGCAG
AAAGATTAGCCGAGAAGCGTCCGCCCGGCGGGGCTCAAGAAAGCTTGGGGACAGCTCCATGTTC
CTTGGGGCGGAATGGCCCAAGAATTGGCCTGGGTAACCCCCTGCCCAGTTCTCTGTCCTCACTTC
GAGCCAGTGCTTAAATAACTTCCCGCCGCCTGCCCTGCAAACTTCCCGGCGCGGCGCCGTTGAGG
CCAGGACACAGCAAAGGCTAGCAAAACCCCGCCGCGGCGCGCTCGGCCCCGGCCCTGAGAGGCT
GCGCGGGTGGGAGGACCAGTGTGGTTTCTGCTCCCACTCGGCTGCCCAGACCCTCAAGACCGATC
CCCCAACTCCTGGGAGCGGGTGCTTCCCTCTGGGAGCCGAGATCTTGCGGGGCCAGCGAGGGCGC
AAAGTGCGCGCGCTGGCCCGCGCGGGGGCGGCCGCGCGTCTCTCCGCGGGGCCTGTCGCCAGGC
CGGCCGCGGCGCGTGAGTGATGAGGGCAGAGAAGGGCGCCCATAAATCGCGGGTGTCAGGGCGA
AAAACTCTCTTTATTGTCTGCGTGATGGATGGGCCCGGGGACGAGACACCAAATACTTCGTATCG
CCTTTAAATGGGAACACATTTTCCGCGGCCATAATTCATGTTTTTTAAATAGAAAGTTTGAAATGT
TGCCTATATTTCACCAGCCCTGACATATTTATGAATCGCTCCCTGCATGCAAATATCATTACTTAA
AGCGCCGGGGAGAGCGCGGGGGAGGGGAGGAGGCGCGGTTCCCGGGGCGTGGGGGTGGTGAAC
CGGGGAAATATCGGTGGAGGGGGGCCATCTCCCTAAAGGGCAGAGGGTGAGGTGCGGTGAATAG
CCGTATCCGGAAACCGAGCGTGCCCCGGGCTTCTTTCCCGCCGCCAGACCCCGCACAGCCGCCCT
GGGACGTTTTTCGAGGCTTGGGACCTAAGACGGGTCCCCGGACCCTGCTGGGAAACCAGGGGGC
GTTTTTCGTCCCTCTCTGAGGCCATTATCCAAGCTGAAACCACCTTTTATAAGAAAAAAGTTTAAA
GAAAAAAAATTTAACAAACACAAATAATAATAATAACCCAACATGTTTCGCGTTCTTTGCCACTG
CATCGATTTTATTGATTTTCATTCTGCAAACCTGGGAATGTGCGCGCTCGGGAGGGGAGGCTGTC
AAAACCCAGTCATCGCGGGGACTGGATGGGGGACAATTCTCCGACGCCGCGGCCTGGACCCGCC
CCCACCCAACCGGCGCACCACCTCCCAGGCACCACGGAGCCTGCGCAAGCTGCGGGGCCGTAAA
GGAACCGGCCCCCAATCTCCGGGGCTTCAGTCCCGCCCCTCTCTCCCCCCAGTGCCCTGCAGCGCT
CCCAGGGTCCGTGTCTGCCCTCCCCAAGCCCCCACTGTCAATACAATCCCTGTTAAAAAAAAAAA
ATCTTTTTAGAGCCAGGCACGTGCCGTCGGCCTCTCCCCTGCCCGCCTGGCCCAGGAGGCGCCCA
CACCTGCGCCCTCACTTGCCGAGACTTTTACCTGTGCAGTCCCCGGCGGCGCCTGCAGCCGCGCA
GGTAACCGAGAAATGGAAGGGAGGGCTGGGGAGTGAGAAGGACAGAGAGCACCGCCCGACACA
ACCAGACGCGCGGACCCGGGGCCTCCTCTCTGGACCGGTGCTGGGGCCCTGGTAGAACCAGCTCC
GGGAGCCGAAATTGAGCCATGAAGAGTGGGCCGAGGGAGGGGCGAGGGGAACGGGCCTCAGGA
GGGAGGGGCGGGCCCGGCGGCACTTCCGTTGGGGGTGGGGCTGCGGGCTGCCGCGCCGGTCCCG
CCCCTCCGCCGCGCGGGCCCGCCCGCTCCAGGCCAGGCTCCGCCCCCACCGACTCGGAGCTGGAT
CTCAGGCCACGGCGCCCCCCCAGTCGCGCCCCCAACCCCGGAGGCCTCGGGCTGAACCGGGTTTC
GCAGGAGGGCACAGGGCG
CGI: 356 range = chr10: 88112905-88117344
CGTGAACGCCCTCGCCTCGCACCTGTGGCGCCTGCCGCGGGGCCCGACCGGAGGCAGTGGCTGTG
GACAAGGGCGGCCCCGGCGTAGGCCAGGATTTCCCCGCAGCCGGACCGGCCCAGCTCCGCTCCTT
TCCTGGGCGAACAGCGCCCACCTCCGGCCCAGGCGGCGGCGCCTCCGCCCGCGGCGCGGACAGC
GAGAGAGAAACCGGCTCCTCCAAACCTGGTGGAGGAGCGCTGCAGAGAAGCGCCGGGCTGAGGC
TCTGGGGGCCGCGGGGCGCGAAACGCCGCGCCTACCAGGTTATGCCCGGGAGAAGGAGACAGGT
CGGGCTCCAGCCGCTGGGCGCTCCTCACCTCGAAGCGAGGAAAACACCGGGCCACTGAGATCAG
GGGAGGTGCGCCCGATACAGGTACCCGGCACGCAGACACGCCCACCCCCAAACACCGAGAAACA
AAGACTGCCCCGAGCCACGCACGCCTGCGCGGCCAGCGCTGGGCGCACACCCGCGCCCACACAG
CCCGAAGGGCGCCACGCAACCCGCAAGAACACCAGCTCCCCGCGCTCGCAGGCTCTCCGTGCCG
GCCCGCAGCGCAGAGCAGTCCCGGCGCCGCTGCCTAGGTGGGCCGGGGAAGGCGCGCCACCGCG
CATGGCACCGCGGCTGCCGAGGAACAGAGGGTGCCCTGCGCAGCCCAGCTCGTCCCAACCCCTCC
GGTGCCCACTGCTGCGACGCCTCGCAGGCCGTGTCCCAGTGGGCCCTGGGCACAGCGGTCACTCA
CCTTCCTGCACAGCCTGGAATGGGTTGTTGGCCTCGATGACCTTGATGGAGTAGGTGATCTTCTCG
CTCTGCAGGATGTCATCGTTGAGGCTCAGGTCGGATACCGCCAACTGGAACACCCTGTCGTCCTT
GGCCGCGTTCTCCTCGAAGATGGCACCTGGAGGAGAGAAGGGATCAAGACCGGACCTGGACAAG
AACCCTCTCCCCGCTTCCCTTGGCCCGAGGGTCAAGGCACTCGCAGACCGATGATTGCCGGGTGG
TGGGAAGTCTGAACTGGGATTTCAGCTTGGCGGGGTAGATTAAGGCTCTCCGATCTCCACCTGCA
CAGGAATATGTTCCCCTCCCCCCACCAGCCCTCAGCGCACACGTGCACACAGTTGACATGAGTTA
CACATGCACATCCCACAGGCTGCCACCAGGTCACACACGTGTCTCACAACCAAGGCACCACATGT
CCTAGCCATGCTCCACACAAGGGACTAAGCCAAGCTGGGGTGGCCCCAGGCAGCGCCCAAATGC
CTTGACATGGTTCCATAAATCCCATCCCAGGAGCCAGGAAGTTCCCCGGGAGGGTGGTGTCCCCC
ACCCAAATCCCAGGACAGAATGATGGGTCCTTCTCCATGCACGTCCCCTGCTGAGTGGAGCTGAG
CGAAGCAGGTCTACAGTTGGCTTTCCTGCGCAGAGGGGTCTTCCTTAGGCAGGGGCTTCGGATGG
CCTGCAGCTTGGATGACTGTGACGCTGCCCGGCCTGGCGCCCTGAGCTCAGCCTCCTACTCTCCAT
CCTTCCCCACTCCCATTTCCTGTACGTCTGTCGAGGGGAGTGGGGGAAGGGGACTTGTGTCCTAG
GCGCCTCCACGTTCAGATCTAGCGGAGCTGGGGGTCCCAGAAGGAGCTGCTCTTGAAGGGCTTTC
TAGAACCGCGGACAGCTCCTCCAGGGCGCCCTCCGGCTGGCGCTTTGTGCGCCTAGCACAGCTTC
CACGGACCCTGCGCTTCCAGCCGAGATCAGTGCGACCAGCCCGAGCCTAGTCCTGCAACCTGGAC
AGCTCTGGAGTCAGCCACCCACATCCCGCCTCACCAAGCCTCGAGGGTCCCTGAGGTCCCGGAGC
TAGGCCCAGTGATCCCTCGAGCTGGGGGCTGGGTAGGAGGAGGGAGGCCCGGATTCCTCACTAC
ACCCGGAGCCGGCGCCGCAATGCTGACCGCGAGACCCGCACCCCTCCAGGGCGGGAAGCAGGTT
TGGTCACGGGCCCCTGAGGAGGAATCGTAGCTCTCCGAGCCCAGGCCTAGTCCTCACCACCCCAC
TCCACCACCCACCCTCCCGCCTTGTCCCGTCCTCCTCGCCTTCAACACCTCCCAACCCCCAGTTTC
CCTGTGCTCGCTGGTCTTTCCCAACTTCCGGAAAGACGACTGCGGAGGGACTCCCCCAAAGCGAC
CGCTGTCAGCCCCCCAACTCGGCCCAACTTCCCGAGATTCCTCCCGTGTTGCTCCCAACTCCCACC
CACTCCCCCTCGGCGCGCCCACTCCCCCTCGGCGCGCCCCCTCCTCCTCTGCGCTTTCATCTTCTCT
CCCGGTTCTCTCTCTCTATCCATCTCTTCCCGCTCAGCATCCCCCTACCCCCCGCTGCCCACGCTTC
TTCCCTCGGCTCCCACCACCCAGACCGTCTGGGCCCCCAAGACTTGGACCACGCAAAACTCTAGG
ACTGTCCAGGATGGGGATGCAGTCGCCACAACCAGATACACACGCACTCACACATACACCAAGG
GCTACACAGACACACACCGGAGAAGCAGGCAGACACTGTGTCCACTCTAACAGGCTGACAGACA
GTCGTGCACGCACCAACACACGCTCAGATGGCCCCACACACCCCAACACCCTCTCACAGTAACAC
CGACACCGTGCCAGGCGCACACAGCGACACACGCTGACAACACGCACCCACACACATTCACACA
CGGTTCCGGTCCGGCCTCTTTGATCTTGCCCTGCCAACTTGGCGAGCCCCCCGCTGTACCTCCCCC
TGCCCCCCGCTGCTCTGAGCTGCGCAGAAGGGCCCTCCCGACCCGCCGACGGCCCCGCTAAGGGC
GCGGGTCTCGACGCGCGTCCATCCCGGTGTTCCCCGAAGCGTCGGCCCTAAGTGTCGGTAGAGGC
CGCGGGGCCCCGCGCGGAGATCGGAGCCCCGGGGAGCGGCAAGCAGCCAGCGGGAGCGCGGCG
CGGCCGGTGCACAGCTCCTCCGCCGGGCGGCGCGGCGCGGCCCTTCGGGGGAGCACCGCCCGCC
GAGCCCCTCGGCCCAGGGAAACGCCAAGTTTGGACGCCGCGCACCCCCTGCCCCGTTGGGGCCCC
CGCCCAGCCTCGGCCCGGCCTCCAGCCGCGCTTACCGATGTGGATGATGGAGTCGGCCCGCACCG
ACACGCACTGGCATATCCAGGGGAGAAGCCACAGCGTCAGCGCTTCCATGTCCCCCGGGCGCGC
GGCTCATCCACCCGGGCCCGGGGCGAGGCCGAGGGCAGCGCGAAGCGGGGAGGCGCTGGTCCCG
GTGCAGTCCCGGGCCGCTCCCCGGGAGAGCCGAGCCCGCCCGTGCGTCTTCCCCCGCGCGCCCGC
CCCTGCGCCCTGCGCCCGCCCCAGCCCAGCCCAGCCCAGCCCAGCGCGGTCGGGCTCCCGCTCCG
GCTCCGGTGGCGGCTGCGGCGGTGCTGGCAGCTTGAGCCCAGGCCGGCGCGGCGGGGGCGGCCC
GCGGCTGTGGCAGCGGCGCTTCCCGCGGCTAGAGCGGCTTCGGGGGAGGCTGAGGCGGTGGCGG
CGGCGGCCGGGCCGGCGTGGCGGCTCTGGGCTGGCTGTGTGTCTGAGCCCCGGCGAGGAGCGAA
CTGCGGAGGACGCCCCCTCCCCTCCCCCTCCCCCTCAGCTCCGCTCCCCCTCCCCTCCCTGCCCGC
CTCCCTCCCCTCCGCCCGCCTCTCTACCACGTGACTGCGGAGCCTCAGCCTCGCCGCGCGGCTCTC
GCCCGCTCGCGGCTCGGAGGGGGCGCCGGGTGCTCCACGCTCCCGGCTCGGGGGACGCTCGGAG
ACCGGCACAGCGGCGGGGACCGGCCTGGGCTGCGGCGCGCCGAGCTCTGACGGTTCGGGCCTCC
TTCTCCGGTTCGCCAGGGGGGGGAGAATGGAGGAGGGCCGGCCGCTAACTGCCCTCGAGGGGCT
CCCGAAGCGCCCTGGCGACCTCAGCAGCCGCGCGTCTCGGGTCATGTCCCCGGCCGAAACAGACC
GTTCCTCGTGGAGTGGGACCTGGGGAGCAAGAAGAGAGACCGGGGCGGGGCGCCGGGCCAGGC
CCCAGGACAGCTGGGGAGCCAGAGGGGGCCGGCCGCCGCAAGCATCTCAGAGTAGAGCCCGGGC
CGTGGGCTCGAACCCTGGCTGGCCGCGCGGACCTCGGCGGGCGCCCATCCCCCGCTCCATGGGCA
GCGCGCTCAGCCGGGAGTCGGGCAAGAGGCTCGGACTCTGAAGCCCAATTCATGGCCGGAAACC
AGCGCAGCCGTGACCGCGGCGCCTGGACGTGAGGAGCCTTCCAAGCGGCCGGCGGGCTCAGACT
CCGACCCTGCACGGAGCTTTCATGTGCGAGCCCGGGCGAAGAGCCCGCTCCTCGCGGCCACAGCG
GAACAGGGCTCCCTCCGCCTCCGGTGCCTGCCTCGGGCCG
CGI: 34 range = chr10: 94441310-94441717
CGAGGGTTTGATGATGCGGCCCGAGCCTGGCTGTGGTCGCCTGTCGGGGCTGGAGCGGGACCCTC
AGCCGGGCCGGGCCTGGGGGCTAACGTTTTCACAGTGCGCCCTGAGTTTCCTTGGGTTACTGCTG
GGACCGCGCAGGAGGAAGCAAAGAGTTTTTCGAGCTAGACCAACAGGAAACACATTGACGGAAA
TGTTGCCATAGCCCATGGGGTGGCTTTAACTGGCCGCCCCCGCGGGCTGGGTGTGAAATCAGAGG
AGGCCGCGGCTCCCCCGGCCAGGATTGGAGGCTCCTCGCGCAACCTAATGCGGGTGTCCGGGCCC
GAGCGCTTCCCGCGCAGCCAGGCCTTGTCGGTGCAGCAGCCCCGCTCCTCCCCAACACGCACACA
CCCGGTGTTCGCAAGTGCG
CGI: 182 range = chr10: 99068802-99071038
CGCGCCCGACTGCGGGAGCCAGGATCAGGTCCCCAGGGCGCCCTTGCAGACAGCACCCTGAGCG
AAGGAGCTCAGAAGGCAGTCCGGCTTCACCCAGACCCCCAATCCCACAGAAGCGCCGTCCCCCA
GCGGCACAGCACCCCCTCCCCGGCGCGGTGGTAGGGCCCGACGTGACGTCACCCATTGTTTACAA
ATCAACCCGAGCCGGCAGGATTCCGGCTCCCGCGGCTGCAGGCGCGCGGCTAGAGTGCCTGGCG
GGCTCCGGCTTCCGCGTCCGCCCCGGCCCCGGTCCAGACTTAGTCTTCAGCTCCGCGCCCGCTCCG
CCGCGGCCCACCGCGCCCGCCGGCAGCCGAGCCCCCAGCGACGCCCGCACAGCTCCGGGTGCCC
AGACAGGGGGCCATGCCGTGCCGGAGGGAGGAGGAAGAGGAAGCCGGCGAGGAGGCGGAGGG
GGAGGAAGAGGAGGAGGACAGCTTCCTCCTACTGCAGCAGTCAGTGGCGCTGGGCAGCTCGGGC
GAGGTGGACCGGCTGGTGGCCCAGATCGGCGAGACGCTGCAGCTGGACGCGGCGCAGCACAGCC
CGGCCTCGCCGTGCGGGCCCCCGGGGGCGCCGCTGCGGGCCCCGGGGCCCCTGGCTGCGGCGGT
GCCGGCGGACAAGGCCAGGTCCCCGGCGGTGCCGCTGCTGCTGCCGCCCGCGTTGGCGGAGACT
GTGGGCCCGGCGCCCCCTGGGGTCCTGCGCTGCGCCCTGGGGGACCGCGGCCGCGTGCGGGGCC
GCGCTGCGCCCTACTGCGTGGCCGAGCTCGCCACAGGCCCCAGCGCGCTGTCCCCACTGCCCCCT
CAGGCCGACCTTGATGGGCCTCCGGGAGCTGGCAAGCAGGGCATCCCGCAGCCGCTGTCGGGTC
CGTGCCGGCGAGGATGGCTCCGGGGCGCCGCCGCCTCCCGCCGCCTGCAGCAGCGACGCGGGTC
CCAACCAGAAACCCGCACAGGCGACGACGACCCGCACCGGCTTCTGCAGCAGCTAGTGCTCTCTG
GAAACCTCATCAAGGAGGCCGTGCGAAGGCTTCATTCGCGACGGCTGCAGTTACGTGCAAAGCTT
CCCCAACGCCCGCTCCTGGGACCTCTGTCGGCCCCGGTGCATGAACCCCCTTCGCCTCGCAGCCCT
CGCGCGGCCTGCAGTGACCCTGGCGCCTCCGGGAGGGCGCAGCTCAGAACTGGCGACGGCGTTC
TTGTGCCTGGCAGCTAACACGCCCGGGGTGGCCACAGCGCCAGCCTCAGACTGGAGGGCAAGGG
GTTCCCTTGAGGGCTGCAGTTCTACTCAGGCTGGTGGAGAACTCTGGCTTTTGGAAGCGAGAGTA
AAAAGCTAATGACGAGGAACCGAAAAATCGCGAGTGTTTCGCGGGTAACTGGGGTTGAGGGCCA
AAATATTTGGAATGAAGGACTTTGGCCCTATTTAAGGCAGATTTTACAGAGCGCACCTCAAACGT
ACAAGTCAGTAGGACTCCTTATTTGGCGTGACCCGACCTGGCCGCGGAGCCTGCATTTCCTCGCA
GCCTCTCAGTGCCCTCCAGCCCCGCGACCATGTGGCCACAATCCACGCTTCTCCGGATCGCGGTG
CGCCGGAACCACGGAGGATGATGCCAGTTACTTGCTTTACCTTTTCAGGGCTGGCTCCTGATCCA
CTTTGGGGGAGGAGAACATGAGTAGATAATTTCAGGGTGCAGCCCAATCTGCCAGACTTAAAAA
AACCATCTTGTGTCTTTGGAGGTGCTGCTTAATACCAAACATGCGGTGCCATGAAGGGACCCTTT
GGGGGTTGAATAGGAGTTAACCCCTGCGCTCTCTTTGCAACTGTCTCTCTTCTCAGAGTGGTGGGG
GAAGGCTGTACGACACGGGTGGGGAAAGGAGGTGGGGGCGGGGAGTATTGAATGGTGGTGGAA
GGGTAGAGAGGCGCGGAGTGAACCCCACGCCCTGTCTAAAGTGTATTTTCAGAGCCGGCCCGCCT
CTCCTCGGTTCAAGGTCACTGTTTCCTGGGCACGCACTGGGTTGCGGGACAGAGTAGCCAGGTTC
TGCCGGTGCTCGGAGAAGAGCGCAGTGTTTTGCAAGTGCTGGAGTCTCCTGAGGACACGCGCGTC
GCCGCCACCGCGGGTGTGGGAAAGCGCGGACGTGCTGGGCGGCTGTGCTTCGGTAGGCGACCAC
CGCCCCTGGCCGCGCTCCGGGCTTTCACGGAAACTCCCGAGACCG
CGI: 56 range = chr10: 101277153-101277910
CGTGCACGGGCTTGGAGACGGGGCGCCAGTACCAGGCAGGGCACAATGAGGCTTGGGGCCCTTA
CAGTCGCTGCAACCTGGAGGTACCGAACCGGGGACTCCGACCTGACATGCAGGCGAGGCTGCGA
GGTCCAGAGCCTGGACCTGGTTCCTAGGGAGCGCCACTCCTTCTGACCCCTGTGGCGTGCGTCCT
GGCGCGCCGCACCCGCCTGGTGCCTCGGCCCTCTCTGCTGTTCCCTGGCTGATTCTGAGCAAGGCC
AGCAGCCCACGGCCCCACCCGACCCCCACATCTCCGCAGGGTCAGGCGAGCCGAGCAGGAACTG
CGCCGGGGCAGTGGTGATGGCTGGGCGTCCCTGCAGCTTCAGGGGCCAAGGGCAATCGGGGGGC
TAGGCCCGCAGACCTGGCCGTCCGCTGACGCAGCGAAGGTCTAGCGGAACCTTGGGGATCGCTCT
CCCAGTGCCGACCGAAGGCCCGGACCTCCAGGCTGGACGCGGCGAAGGCCGCATAAGACGTTAC
TTAAACATGTTACTTAAACAAGACTGCAGTAAACGTTTCTTTCCAAGTGAGAAAGGTCTTTTTCGT
TCTCAGACGGTTTGAAGGTGTTTGTGCCAACGTGACCCCCGGGGAGATTTGGAGGAAGCTTTCTA
CGTCCTAGGAGGCTGAGATCCCACGGAGCCGGTTTACGGTTGAGAGCAGACAGTTTCGAGTAGAT
AGCGCTGGAAGAGACACGAAGAATCTCTCTTGCTTCTTGGAACGCG
CGI: 403 range = chr10: 102974173-102980053
CGCTTTAACTAGACGGGCGGATAAATGGCCCCGTTTCTCCTCAGCCCCGACGCGCCTGCTTAGCT
GCGCGCCCCGCGGGCGCCGCAGCTCGGGTGGGCTCAGCCCTGGGGCCGCCGCCCCCAGCCCAAA
GGGACCCAGCGGGGCCAGGCCCAGGGCCGCGAAGCAGAGAGCGGCCGGAGGTGCCAGGGCCGC
TTCATTGGGATTCATGAGCGTGTTGGGCGGGCGGCCTGGGGCTTCGGGCGCGGGGCGGGGGCCG
AGTGGGGGGGTGCGCACGGCGGGCGCAGATGCCCGGGTGACCGACCCGCCGTGGTCCCTGCGCC
GCCTTTGTGCCGAGGAGGCGCGCGCGGGGACCTAATCCATCAAATTGTCTACAAATTGTGAAGAA
AATTCAAAAACCATATGGTCGCCAGCGCCGGCGCGCTCTGGGCTGCGGAGGGCCGCGGCCGTGC
CCGCACCGGGAGCCGCGGGGGGCTGGGCCCGGCTATTGTCGCCTGTCAGCGGCCGCCCAGGCCC
ATTCGTCCCGGCCTTCATGGTCCGCGCTCCGAATTACAGACCCATCCATCCTAAGAGAGGGCAAT
TTTTGTCGCCCGGGAGGAACCAGCCGCACGCCTGCCCCGCTGCCCTGCCGTCTCCTTCCTTCTCTG
CTCCTTCTCTCTCTTCTCCTCTCTTCCCATTCTTTTTTTCTCCCTCTGTCTCTGTCTCCCTTTCTCCGT
TTCTCTGTTTCTCTCTCTTTTTCCTCTTTTTCTCCGTCCTGTCTCTGTGGCTCTCCGCTTTCTCTCTCT
GCTTTTGGAGTGTCTTTCTGTGCTCCTATTTTTTTTTCCTGTTTCTCTCTGTCTCTTCGTTTCCTCCTG
GACACGCACGTTCCTGGCTCGGGCCCGGGGGCCCCATCGCCGCCCGAGACCAGATGCGCATTTCT
CACATCAATTTTGCGGAGAAAGAGAGCCCAGCGGCCCGGAGGCGGGACCCCACTCCGGTGACAG
CGGCTACTCGCCAGCCCAGCGACAACAAAAGCCCGGAGAGAAACCCGCTCTGTCCAGGAAAGTG
AAAGCGGCCTCAGCCCCCGCCCCAGCCGGCCTTGGGGCGCCGCAGACCCGAGAGAGTCCTTGCA
AGATCCATACCTCTGCCCCCAGCGGTGTGCCGGGGTGTGGAGGCAGAATTGGTTAATGTTGCCAG
AAATTCACACCAGCTCCCAGACGCTTGGGCTTACAGTTCCAGGTGCCCACATTGAAATTCAGCCT
GGCCTTGATGCCTTTAGCCTTTGGCCCCCGACTCCCCAAGGATGAGGCCCTCGGGGCTAGAATCG
AAACTTGGGAACCCAAGGCTCCACCCCGCGTCTGACTGAGTGATACCTGCAGCGCTCCCGGGGCT
CAGCCCTGTTTTCGGGGAGTGCAGGGAGAATGCCCTTGGGTCTGGGGTGGGATGCAGGAAATGC
AGGGCAAGACCCCCTCCCACCCTTTGTTCAACACAAATTAATGTTGTACTGTGGTGTGCCCCCAC
ATACACAGGCATTTTCTCCCAGCATTTCACGGCTCGTTTTTTACCCCGACCTGGCGAAGCCCGCTT
CTCTCCCGATTTAGCAGCTGCCTCAGGTTTATGGGCAAGAGGCGCCTCCTCCTGCTGCTTCCCCCT
CGCCCTTCACTCTTAAGCCTGTCCTGAAAATCCCCCCCAAATCTCCAAGGCTAGGGCGACCGGCA
CGAAGAGCGGCAAGAAGCCCAGCCTGCCGCTGCTTCTCGGAAACGGGCCCACCACTCAGACACG
GTCGCCGCCGGTTCTAGTAAAATGGCAAAGACTTTATTTATTGGAGAAAGAGGCCTTGGGTACAG
TCTAGGGAGGAGAGGGGAAGGGAGGAGCAGGGAAGGGGCACTCGCAGCCCACTCACATCCAGC
CACCCTCCTCTGGCCAAGTCGGGTAGGAGTGGGGTGTTCGAAGTGGCAAGAGAGACCCAGAGCT
GTGGAGGAGGTGAGGTGAGGGCTTGGGTCGGCCTCCTCTCTGTCTCTGTCACATGCAAAAAAATT
CTAAAAAGAGTACAAAATAATTATAGGGCTTTGGGTTCTGTTTTCGATAGTTGGTTGGTTGGATTT
CCCCCTGCATTGTCCCTAAGAGCCGGACTCCAACAGTCAGTCCGAGGAAATGCGGGAGGGAGGG
AGGAATCTTAAAAACAGCATTAGTGGGCATTAGGAGGCAGAAATGGCATCGAAAACAGACTTTT
CTCTCAGAGGGTGGGGAGGAAAGATCGATTTTGAAACTTGCAAGTCAGGACCTGACTGCCCACCT
CTTCCCCTCCCACATGAGGAAAAAAGGCCCGAGGAAATAGAAGTCAACTGCAATAAGGCCGAGG
GAGGCGAGGAAGGAGCGGGCCGGTGCATTCGCCACATGCAGAGAAAAATCAAAACAGCCGGAG
CTGCCACTCCCCTAGACCGCAGGGTTAGTGTTCCCCGCCCTGGTTTCTGTCTCGCCCTCAGCAAAC
CCGGCAGCCCGAGAGCTTTGAAAACATCGAAATCTCCGAGATTTAAAAAATAATAATAAAAGAA
AGGAAAAATCAGGCTGCGGTGGGAAGAACCCGCTATATAGGTTTAAATATTTATATAGAAGCCA
TACAGAATTGCACGGCGAGGGCTTCCGGGGCGGCGGAGGCCGCTGGTGGCGGCCGGCCCGGCCG
GCCAGCCTGGGTTTATTGCTTCGAGAGGGAAGAGGCGGCTGCCAGGAGCCGAGTCCGGGGCCGG
GGACAGGGGAGGAGGCGGGAGTTGGCAGAGGAGGTCCCAGCTCCCCTCGGCGGTCCGGTCCGGG
AGGCGTTGGGCTTTCGAGCGCTAGGAGCCCAGGGCGGCGGAAGACCCGGGGCGCCGCTCAATCG
TCCACGTCGATCTCTTCGTCTTCCTCGTCCTCCGAGCAGTCCTGGCTGCTGGCCGGCTGGTCCGTG
AGCGGAGAGGCAGGCGAGAGCTGCAGGGCGCCAGCGCCCGGGGCCTTCGGGGCGCCTGGGGGG
AGGACCGGAGAGCCGGGCCTCGACTTGGCCCTGCCGCAGCCGCCGCCACCGCCGGCTGTGGCCTC
CGAGTTCTGCTCGAGTTCGGCCAGCGCCACGATGTCCATCTGCCCGCTGGGGCCCAGTTTCTTGGC
GGACTCTACGTCGGCCTTCATCTCCTCCAGGTCCCGCTTGAGCTTAGCGCGCCGATTCTGGAACCA
GGTGATGACTTGCGCGTTGGTGAGGCCCAGCTGCTGCGCGATTTGGTCGCGATCGGCGGGGGACA
GGTACTTCTGGTATAGAAAGCGCTTTTCCAATTCATAGATCTGGTGGTTGGTGAAGGCCGTGCGC
GACTTTCGCCGCTTCTTAGGGGTCTGCCGCTGCCCAAAGATGGTCATACCGTCGCGGCCTGGGAG
GAAAGGACAGTGTAGGCTCGCGTTGGGAGAGAGGAAGCCCACCCTGGCTCTCGCCCCCGTAACT
CCGTAACCCACCCAGGCCTGTCCGGCCTGCAGTCTACCAATGTTCCCTTCTCAGATTTAATTCAGC
CTCCAGGACCAGGGCAAGTCTTGGCTATGGCAAACCATCTCCTGTCTTTGGTCTCCCAGAAGTCC
CTAGGAAAAATTGTTTTGGTCTCTGCCCCCTACCTAAGAGTTGGTTTGAAAGACCCAAAGTCTCTA
AGGGTATGAGAAAGCCAGACAGAGACACCCTAATTGCGGTGTGAATGTTGGTTCTCCATCCCTTC
CCAATCACTTCAACTCTACCCTACCCATAAGGTCCCCGGCAAAAGACTCTCGATTCCCCACGCCG
AGTAGCCGGGCTAAGAGCGCCCAGGACACTGGGAACGCAAGGGAGCCCGGCAAAGCAAGCAGA
CTGCGGAAAGAGCTTGGCCACCCTTCTCCTGCCACCCCCCAGCTCTGGCCTACCCAGAGGTCCCT
GGGAAATAAGGATTGGGTCCCGTCCCACATATCTTCACTCCCATAAGCAGCTGCTCAGGCTGGAA
GGCCCTGGCGGCCGGAGAGGGCAGAGGCGAGCAGAGGCATAGGGGACGAAGCGCGCAGGGCAC
AGGGCGCGAGGCGCTGGGTGCAGGGGAACCCAGCAGCTGCGCCTACCTTCGGCTGCCTGCAGAA
CGCTGACCTCCAGCCCCTTAAACGTCTTGCTGGCGAGCTCCTCCAGCGCGCACAGCGGCGAGGTC
TGCGAGAGCAGCGCGCGGCCCGCCAGGGGCAAGCCGCCCTGCGCGTGCTTGTCCGCGGCGGCCA
GCAGGTGCGCCGCCCCGCACAGCGAGTAACTTCTCCGCACAGACGGCTTGTTGAGGATGTCCTCG
ATGCTGAACGGCGTCAGTGGCTTGTTGGAGTTGGCAGGCGGAGGCAGGTGGTCCAGCGGGCTGC
GCCGCCGCTCCTCCCCCGGCGCCGCCTTGCCGTCCTCCTTGGAAGTCATCTCGGCCTTGTTCGGGG
TCCCGGCCGGGGCGGCTCGGGCCGCGGGGACCCAGCCCGCGGGCAGCTCGGGCGCCGGACGGCG
CGGGCAGGCAGCGGCGGGTGGAAAGGAGGCCGCACTGGGCAGGGCGCGGGCCGGGCGCGGGGC
CGATTAGCGCGGCGGCTGAGGAGGGGAGAACAGCGCGGGGCCCCGGGAAGAGGGCGCTGACGC
GGGCGCCGCTGCTGCTGGGGCTTCCAGCAATCCGGGGCCGGAGCCGGGGCGCGCTGCGCGGGGC
TCCAGTTTCGCCAACCCCGGTGCTATTTAAAGGTGTCAGGTGGCTCGGCAGTGGCTCCTGGCCCC
GGGTCCTGGCGGCCGCAGTTGGAAGAGCCGAGGCGAGGGCGCCGATCCCGGACTGCGAGGGAGG
CGGAGAGACGGGGCAATTGACTATTGCTAACAAGTTAGCCTTGATCGCGGTGTAATCAATTATCT
GCAGCGGCACTTCTTTCTCTTTCTCTCTCCTTTCTCCCTTTTTCCTCTCTTCTTTGCCTCTGTTCTCTC
CTCCTGTTCTCGCTCTCCCTCCCTCCCTCTCTCTTCTCTCTTCTCTGCTCCCCCCTTCTCTCTCCTTCT
CTCCCTCTCCCTCGCCCTCTCTTTCACTCTCTGTCTGTCCAATGCAGGCGGCTGTAAGTTGGGAAG
CTCATTAATTAGCGGGCCGGGGGTAGGAGGAGCCGGCTGGAATTAGCCTGCCTAATGCGCCTGTC
AGTCCGGGCGCTGCGCTGCGTTCGGCCCGAGCTGTTTGTAAATTACATTAGCTGCTCCTTTATTGC
ACCCGAACCTCGGGCGACTGAAAAGCCACCGCCCCCACCCCAAACTGCGAGCCGCGCTCCTGGC
GCACCCGCCTCCCGCCGGCCTAGCTGCAATGACCGCACCGGCCCGAAGGTCTCGGTCTCTCCGAC
CCGGGATGTGGAGCCCGAAAGAGTGGTGGGAACCCCAGCCCGGGAGGGACGCGGCCGCCGCTCG
GGCCAGGTAGGACCAGGAGCCCGGGTGGGCCCCGCTGCCACCCAGAGTGGACTTTGCGGAGCTG
AGATCAAGAGCCCAGCGCCTCTACCTCTGACCCTCTTGCCCCTCACCCCACTGCCTGGGCGACGC
GCCTGGGGAGAGGGAATCTGAAGCCCTCCTGCGCTGGCCCGCAGGAGGTCAGAGCCGGCTGCGC
AGCCTTGGGTGGGCGACGCGGGAAGGGGGTGGAGGACCCCAGGGGTTGGGGGAGGGGGCTAGT
CGGAGAGCTCAGCCTTCTCCACGCGGAGAGGCGGAGGAGGGAATTGTTGGCGGCTCTGGACGCC
TCTCCCCGGCCTGATTCCGAAGCGGCCGCCCTACCTCCTCCGGCAATCTTGCTCACTTTCCTCCGG
CAGCCGCTCCCCAGGCTGGCTGGGGCCACCAACGGCCTGATGATGGGCGTCTGAAGAGTAGCCA
GGCCAGGTGCCGGGGCTAACGGGGCCGGCTCCGTGCCTCCCTGCTGCG
CGI: 295 range = chr10: 106389558-106392802
CGGTCACGAAGGATCTCAGCTGCACCAAGCGCGCAGGCTACACTCCTGCAGTAGCGGATTGGGG
ACCAGATAAGCCCACGCGGCTCCCGCCTGAGTACCCTCTTAACTGGCGGCCGACACTTTGAAACA
ATTGAGTCCGTTCAGAAACGGACTGGGGAGAGTGAGGGCACGACCTCAAAATCCTAGGCAGCAA
AATGAGGTCTGGATCCTCCCTGGAAGAACAGACATCTCGACTACTCTCTCGCCCCACCTCTGGCC
GGGAGGTCGGGGCAAGGAGAATCCCTCAACCGAGAAGATCTGGCAAGTCCCCGATCCCACCGGG
GCCTTCCGCCTCTTTTCCCTCATCCCCTAGGAGGCTTTATCAGGTTCTAACCGCTTGTAGTGGGGG
CCGTGGGTGCTCGGGGCGCCAAGAGGAGGGGGCTGCCGAGAGCGGCCTGCCTAGTTCTCAGCAT
TGCCTCTTTGACCACTCGCAGGCTCCTGGAAATGCAAACACAGCCCGTGCGTGATCCGCACGCGA
CCAAAAGCCGAGCGCACACCTCCCGCCCCCGCCCCACTTGCCATCGCTCACACTAACGTGCACAC
ATGCTCGTGCAAACGCGCACGCACGGAACCGCGCGCCCCCTCACACCCAGCCGCGCTCCCGGCCC
GGCTCAGCCAGGTGCCCCCACGCGGCAGCGCCCGGTCTGCCCGCCGGCCCCCTGCGAACCCCTCG
CCTCACGCCCGCCTGCCTGCGCGCACGCACGCGCACGCACGCATTCCCCACGCCCCCGCGCGGGG
GCGCAGGCCTTGGGTGGCGGGGTTTGGCCGGGAGCCGGCCGGGCCAGGGAGCGGCGAGCAGAGT
CCAGGGTGCATCCCGCGCTTGGCTTCCGTGCGCAGCCTCCCCGCGCGGAGGCACCTGGCTCCCTC
TCGGCTCTAGAACGCGAACAGTTGGCGTTGAGCACACAACTCCTCCTCCTTCTCCCCCGAGACCC
CTGTACCCTCCCCGCGCGCCATCTCCCGCCCCTCATAACAGGCCCGAAGAGTAAACCCCCAAACG
CGTCCAGAAGCGGCTCCCAACTCTTCGCCGGGAGCACGTTGGAGGCTGGATTTCGGAGAGACTCG
GGATTGGGGTCTATTGCCGGCCCCTCCTGGATCCGGCTTGATTTTCATATTTTAAGAAGATTTTCT
TATTACCTTTGATTACTCCTTTTTACCAAATTTCTGGAGGAGCTCAGTGGGGAGGATTGGGGGGA
ATCTGATTTTAAGAAAGAAAGCACCGAAGGGGCAATTATTAATTTTCCTCGGGTTTGGAATCACC
CTCTGGACAAGAGAACGGGCGAGCGGGAGCTAGGAGGGAAGAGTGGAGAGGACCGGCGAGGCG
CGCCAGCCGGAGCCACCTCCTTCCCGGCCGCCCCCTCCCCACTCCCCCTACACACACACGCTCGCT
CGCTCGCCGGCGCGCGCACACCCCCCGCGCCGGACCCGCACCTCGGCGGGCGCCACACACTCGG
CAGCCCGAGCCGCGGTAGCCGCAGCGGGATGGAGGCGGCGCGCACGGAGCGCCCCGCAGGCAG
GCCGGGGGCGCCGCTTGTCCGGACGGGGCTCCTACTCTTGTCGACGTGGGTCCTGGCCGGCGCCG
AGATCACTTGGGACGCGACAGGCGGTCCCGGACGCCCGGCGGCCCCGGCTTCGCGGCCACCGGC
GTTGTCTCCACTCTCGCCGCGGGCAGTGGCCAGCCAGTGGCCGGAGGAGCTGGCGTCGGCGCGG
AGAGCCGCCGTGCTGGGGCGCCGGGCCGGACCAGAGCTGCTGCCCCAGCAGGGCGGCGGCAGAG
GCGGTGAGATGCAGGTGGAAGCCGGAGGGACATCACCGGCAGGCGAGCGGCGGGGCCGGGGCA
TCCCAGCTCCTGCCAAGCTTGGCGGCGCGAGGAGGAGTCGCCGGGCGCAGCCCCCAATCACCCA
GGAACGCGGGGACGCCTGGGCCACTGCTCCGGCCGATGGTTCCAGAGGAAGCCGTCCCCTTGCTA
AGGGTTCCCGGGAGGAGGTGAAGGCGCCGCGGGCTGGGGGGTCGGCGGCTGAAGACCTCCGGCT
GCCCAGCACCTCCTTCGCGCTGACCGGGGACTCGGCCCACAACCAAGCCATGGTGCACTGGTCGG
GACACAACAGCAGCGTGAGTACCCACCCGGCGGCGGGTCCGCCTGTTTCCTGACACCGAAGGGG
AATGGGGGGGTGGGTGGGAGCGAGGGACAGATAGTTGCTCGGGGGTCGAGGCGGGGGACGCCT
CGACTTTTCGAGATAACAGGTTTGTCCGATTCCCCCCACGCCCCCAACAGGTTAACGGAGTTTGTT
GTTAGACGCTGTGTGTGTTTGTTTACTGGAGACCCTAGGCTTCGGCCGCCGGGAGGGGAGTGGAG
GAAGGGGCTTCTTAAAGATGAGTGGGGGAGGGATCTGTGCTCACTTTCTCCAATACTTGGCTTGG
AGGGTCAGTTTTCCTGTTTGCGGGGTGCTTGAATTCTTGGATGAGAAAAAGGGCTGACTTGGGGC
GGGAGCCGCTGAACAGACCGATTCCTGCGGCTGCCGGGCTCCCCCTACCCCCACCCCACCCCCAC
CCCCCCTCCCGTCACGCGAAAGGGAACCCGGAAGGCCATTCGGACTCCCAGCCTCCTGCTCGCCT
CGGGGTCGCTGCTATCCGCTCCAGGCGCGCAGTCCTCCAGCCCAAGAGGGAGGCTCGGCCAAGTC
GGCCCCCAGCCTTGGCCTTCAGGTAACCCCGGTTCCTCCTTCAAAGTCCAGGGAGCGGCCCTGGA
AAGCCGTAGCAGAGGCCGTAAAAAAAAGTTTAAAGCGTGAAGCGAAATTCCACACCCATCCAGC
GCTCATGCCACACCGCTCGCCCTCACGCACGCACAGGCACACGGAAGTCGCTCAGCAGAAAACT
CTCGACTTCACCATTGCGTCCTGCGCCACAAACGGCTCCCTGAGCGGAGTTGGGTGCCTGAGCTT
CCTGCACACCTCTCCACCCGCTTTTCCCCGCCACCGGGTTCTCCTGGACAAGGATGTACCGAGAG
GTGGCGTCGTTGAGCCCGGTCTGGCCTACTCCGGCATTCCGAACTGGGCGCCCGACTGAGCATCG
CGCCTGCCTGGCAGCTGCAGCGGCCCGCAGCGCGTGCCCGGAGGGGCTCCCCCTACCTCGCCGGC
ACCCAGCGCTGTGCAGCCAAGCAAAGAAAAGGCGGGGGACAGGTTCCCACCATCTCCCCGACTT
CCCTCCCTCCCGCG
CGI: 56 range = chr10: 124891898-124892607
CGGGCGCTCATTCTGAGCCGCGACGCCCCCTCAACAGGCTCCCGGCACCGCAGAGCCAGAGGCC
ACGCGGTGAGAACCCCAGTGCGTGCGCTCTTCTTGTGTGAGAGGCCGGAGGTAGAGTTGGGAGA
AAGAGGAGCCGGCGAAAAGGTTAGTGGGATCTGCTTTCCCTTCTGCGGCCTTCTGGGCCAGCTAG
GGCCTGGGAGATGGGGACCAGGGTCGGAGGCTTGGGGGCGGGAGGGCGGTGCTGCCGGCTGCTT
CCTGCAAGCGGTTTGAAGAGGCATCGCGTTGTCCGGCCGCCTGCGCAAGGGCCACGCGCTGCTGC
TTTCACCCCCTTGCGCCCGCGGGGCACCTGGACTCCCCTTAAGAGTCCCCGTGGGGCCTGAGCTTC
TGCTGGGCGCTACGCTGCCCTCCACGATTCCATTTTGCCCCATTTACTCGGGTCGTCCACTTCCCG
CGGCTCAGTGACCCAGCCAGGGGGCGTCTTCCGCTAGCGGTCCAAATGTAGACTCCGCAAAGAG
GCTAAGAAGACCTGTCTACCCGGGTGCGCGCGATGGGGTCAGGGTGTAAGAACCAGGAGCCTCA
TAGAAACCTCTGCTTGGGGTCCAAAGCCCAGCCGGGCACTCCCAGAGGACAACATCCGCCCCCGC
ATGTACACGTGGCCCCGCAGGACCCAGCCGCAGGGCTCCCACGCTCGGCTCCCCGCCCTCTCG
CGI: 32 range = chr10: 128863581-128864054
CGACCACTTCCGTATGAGGCACGATTCGTTGGCATTCTTCACCTGCAGCAACCGCTGTGGGACTA
GGCTGGTTCCCCAGCCCCGGCGGGGTGTGAGTCTGCGACGGCTGCTCACTACATTCGGGGGACAG
GCACTGCATCGGGGGTGAGCAGGCCAGGGCAGTTTCCGAGGGCGCCTGGAGCCCGTTGAGATGT
GTGGCTCTCCTCCGCTCGTCATCTGCCGGGCAGAGTGACCACTGGCTGGCGGCTGCAGAGTCTGA
CAAACCAGGCTCGGGGGCCCTGGCACTGAGGGCAGGTGCGTAAACGGTGGCAACCTCCGTTTTA
AACACCCTCCTGAGGGCAGGTGGAGCGGGCTCCTCGGAGTTTGGCCTCCCCCGGTCCTGCTTGGC
CCTCCCGAGCAGCTGGTAATCGGGCTCTTCGGATGGAGGCCGAGTGGAGTTTTGGAGCGCAGCTT
CTCCATAGCTGAGCGGCTCCG
CGI: 38 range = chr11: 22319439-22319953
CGCCGCCGCCGCGGCCAAGGGCACCTGCAGAATGTGAGAGAGCCTGGGAGGTCTTGCAGCTTGG
TGTCACCCCACCACCCTAATCCCCGAGGGTGGGGGGAGGTCGACGGCCCTCCTCCCAACCCCGAC
GGGTCCTGAAAAATGGGAATGGGTCAGCATCGGGGGTACCTAAGCCGCCAAGTCCTTGACTCGC
CCCTGCTCTGCCGCGCAGGGTGCTGGAGAAGAAGCAAGACACCGGGGAGACAATCGAGCTGACG
GAGGATGGGAAGCCCCTAGAGGTGCCCGAGAGGAAGGCGCCGCTGTGCGACTGCACGTGCTTCG
GCCTGCCCCGCCGCTACATTATCGCCATCATGAGCGGCCTGGGCTTCTGCATCTCCTTCGGTATCC
GCTGCAACCTGGGCGTGGCCATTGTGGACATGGTCAACAACAGCACCATCCACCGCGGGGGCAA
GGTCATCAAGGAGGTGGGCAACGTCTGGCCGCCCTGGCTCCTGCCCTTCGGCCATGCGGCCTCG
CGI: 98 range = chr11: 31776637-31777992
CGGGAACGTGAAGCGAAGCTCACCACCGGTGCCCGCTACCCCCACGCCGGCAACTCTGTGCTTCG
TTTGGCCCACGTGTCCTCCCCACGCAACTAGGGGCGACGGTCCTGTGCCTGAGGTGACAATCACC
CCCCAAAAACCTCACTTGCGGGTGGGGGGGTGGTGATTGCCAGTTGACCCAGAATTTTCTAGCAA
CTAGGCGCGTTAAAGTGAAAAGCCCCTGGACTAGCTCCACCTCCGGCCCCAACCATCACAGGCGG
ACCCTAGCTGATTTGACTCTCACTTCCCGCCCTCAGACTGCCGGCGACGAAAATCCGCCCGAATC
GGGCGCCACCTCTGGTGGAAGCTGCTGTCCTCGGCTTCTGCCCAACTCCAAGAAGCGGAAGGGGG
GTGTATCCTGCGCCCCAAGCTCATGGGAGCCCACGCACCGGGGAGAGCCCGGAGTGGAGGCTCT
GGGCTCAGCCCTCCCAGGCCTAGGCCACCGTGCCCTGAGTCCCTCCCCAAGACTTCTCGGACTAA
GTGGGCCAGACTCTTGTCAGGGGAGGGATCACCAGGACCTCAGATTGCTGGGGGCGGAGGGGTG
GACGCTCGTGAGTGCGAAGTTCGGGTGTCACTTGGGTGTGTCCAGTGTCTACAAGTGGGAATCCT
ACGGTTTCAAAAGTCAGGCAGGGATGGGGTTGCCTACACTCGGTGGGGTCAATCGCTAGTCTCAC
CAGAGGCGGGAGCGGGCCTGGGGGCCCTCTGAGCTCGCGGAGGCGGCGGGTTCGGGGCCCGTGC
GGGCGGGGTCGCCCAGGGGAGCCCGGGCCCCGCGCGTCAGGAGGCCTCCGCCGGCCGAGCGGCC
GCGCTGGGAAACAAGAAGCGAGCGCTTTGCTCCCTATCTTCCCAGTGTCCGTCCTATATTGTTTTC
TGCTCTTAAAACTGACATGTCTAATTGGCAATTGGTGCCGAATCGTGTCCAGAGGTCTCTTGTGGA
ACATTTCTACAGCTGTCTCCTTCAACTAGACGCTTATTCATGTCGCCTTAATGAGAAACAAAACGT
TCTCTAATGAGCAATTACAGAGCGACAGGATTGTTCCCATAACAAATTCTTGCGAGTGACAGAAG
CATCCTTTGTACCAGATATTTCGCATACTGTTACCGATTTTCCCTTCCCTCGCCCGACTCCATGGA
GGCGCCGGGCATCCGGAGCCGGTCCGAGAGCTCACGGATGCGCCGGCAGCCTCCTCGCAGTCCC
GCGCGCCGCCCCTGGCCCCGAGCCCCTCGGCTCCGTTGTGCTCCTGGGAGTGAGACGCCACGTCC
CATTATCCCGCATATTATCTCCTTGTCACGAGGACAACAAATACCGATTAATCTTCG
CGI: 61 range = chr11: 68274244-68274755
CGGACGCCCGGGTGGGAGGCTCCTTCCAGGACCCAGGCGCCCAGGGGCAACACCGCCGGGCTGC
GGTGCGCGGGTAGCAGCGAGGACAGGAAGTGCACGTTGCAGGCGGGCGGCTGCGGGGCCTGCAG
GAGCGCGACGTCCTCCAGCGCGCTGAGCTCTGGGATCCCGGGGTACTCCCCGAGGAGCTCCCCGC
CGTCGCTGTCGCCCCCCGCGAGCTTCGCCTTGACCTGGCCCTTGCAGTCGGCGCCCAGCGCGGGG
TTGAACGCGTGCAGGACGGGTTCCTTGGGGTCCGCCGGGCCCAGGTACGCTTCTTTGAAGACGTG
GAACTCGTCCTCCTCGTACTTCACGGGGGCCTTCAGGCCGATGTTCTCCGAAGCGAACGGACCCT
CGGGGCTTAAGAGCTCCGTCACCATCGCATCCTCGGGGCTGGGCAGCCCGCCCGGCAGAGGGCC
CTCCTCCATGGCGCAGGGCGGCGGGGGGGGATGGCGGGGGCCGCGCACCTGCAACACGCG
CGI: 269 range = chr11: 117983446-117987106
CGGACGGGACGGGGGACGGGGGCGGGGACTGGCGGGAGGGGGGCGGGACAGGCTCGCTCTCCT
CCTACTTAGCGAGCTCCCGGGGAAAAGCAACGGTGTCCTCCTAAGCCTGAGGCCACCGCGACCG
AGCCGAGCCAGGCTAAGGGACCAGTGTCTCCCTGCCCCCCCCCCACCTCTAGGTAAGAGCGCCCC
GTCCCACTACACTTGCGGGTCCCATTCGCTGCGGTGCTAGGACTGGATAAGGGGAAGTCCCCGGG
GCCTGGCGAGAGCCCTGAGATCAGCTCTAGGCTAGGGAGCTCGGCAGAAACCCGTGGGGGAGAG
AGGGCACCCCAGGTGAGTGCGTGGGGGCGAAAGGAGCGTTATGGGGAGTTTTCCTGTGAGCCTC
TTGCTTTTGCCTTCCTCGGCCGCTGCCAACGCCACCGCCAGAGGGGGCCGTGACGCTCTCCCCCCC
TTGGAGAAACTCTGATCCTGGCCCTATCCAGACTCTCCCGCCGGCACCCAGGCGGCCGGGCGGAC
ACTCGCGGGGTATCGGGCGGCGAGCGCGCGCGCGGGTTTTCGTCCTCCAGCGCCCCACACCTCCC
CCTCTCCGCCCACAGCAGGACCTTGGGGCGGGGGTATCTCCTGTGACGTCTCCCCGCCTGTCCCC
AGGCCGGCCGCCTTGATGGACCAGGAAGGGATTCGGAGTCCGGCTCAGAGTCTTGAGCGTCTAG
GAGGGCTGCCTGGGGGTGTCCAGGAGATGGGAAGCCCGCCTGGGGCCTCCCAGGCACTCCCCAA
GGCCCCACGTCCTCGGGGGCCGGGGAGGCCGCCTGGGGCGCCTTGCCACGCCCGCGCGGTCCCTA
TTGGAATCCCTAGCGGAGTTCCCCGAGCGGAGGCTGACCCAAGTCATCCGGGCTCCCCCGGGATA
GGGAAGTGCGGGCGGGCGGCTGGGTAGGGGCGCCGGCGCAGGGTGGCCGAGTCGCCCGCTAGCG
CTTCCGCCGAGGCAGGCTCGAGTGGGGACTTGGCCGGGCGACCGCACAGCCCTGCCGGGGACCC
ACGGCTCTGGGGCGGGGCTGCATTCTGGGCCGTTAGCTCAGCGGTCCTGGAGCCTCCCGAGGCTG
ACTCATCGGGCGGCGGGCTCACCCCCCAGCCGTCAAACGCAAACGCAGGCGCCCCACCACGCCG
GGCTCCCACGCGGCACACACGCACGCCTCCGCGACACTTCGCACACCTACGTCCCTGCGTCACGC
CACCCAGACACACCCGGACCCGCCCACACGCCGAGGCCTTCCCACGCACTCCATGCCTTACGCCT
CACAGGAAAGCGCCCCGCGCTCACGCAGCCCCTCACACCCGCAGCCCCGCTTACACGCGCCCCAG
CTCACACATGCACCCCAGTCACACAGCCTACCTCACACACGCACCCCAGTCACACACACAACCCA
GCTCACACACGTACCCGTCACACATGAGGCCCCAGCTCACACACGAAGCCCAGCTCACACACGC
AGTCCCAGCTCTCACACACACACCCCAGTCAAACACGAGGCCCCAGCTCACACACGAAGCCCAG
CTCACACACACTGCCCCAGTTACACAGCCCAGCTCATGCAAGCAGCCCGTCACACACACAGCCCA
GCTCACACACACAGCCCAGCTCACACAAGCAGCCCGTCACACACACAGCCCAGCTCACACACAC
AGCCCCAGTTATACACACACAGCCCAGCTCACACACGCAGCCCGTCACACACACAGCTCAGCTCA
ATACACAGCCCAGCTCACACACACAGCCCCAGTTACACACACAGCCCAGCTTACACACACAGCCC
AGCTCACACACACAGCCCATCACACACACAGCCTATCACACACACAGCTCAGCTCAATACACAGC
CCAGCTGACACACGCAGCCCAGCTCACACATACACTCCAGCTCACACACGCAGCCCGTCACACAG
CCCCAGCCACACACACAGCCCAGCTCACACACGCAGCCCAGCTCACACATGCAGCCCCCTCACAC
ATGCAGCCCCAGTCACACACACAGCCCAGCTCACACCGCAGCCCCAGCTCACACACGCGGCCCCT
CACACACATTCAGCCGCCCTTCCCGCAGCTGCCAGGCTCGGAACGGGCGGTGTCTGTGGGGGCCG
CCCCGTTTCCCGGCCGCGCAAACAAGCGGCTCCTTCTCTCTGCCCCGCCAAAGAAAGGCCGCTTG
TCAGGGGCGGGCGGGCAGGCGGCAGCGGCAGGCGCCCAACACTGCGGCCTTGTCCCACGCGGGC
AGCCTGGGCAGGAGCAGGCCGAGGGGTGCAAGGGGCCGCTACCTTGCGGAAACCTCCCTCCCTC
GGCCCTGCGGACCGCGGAGAGGCACCTTTCCACCACCGGACACCCAAGCAACAGCCCTCGCTCC
ATCCGCGCTCTCCCGGCTCCCCCTCTCCTCGGGCCGCCCATCTCTCCTCTCTTCCGCCTTTGGGGTC
CCGGTGGGCCCCAGCCTCCCTCTCCTTCGCCCCATCTCCCCGCCCGTCAGCCTCCCCTCCGCTGCC
CTCGCGCATCCCCAGTGTTCCCGTCTGTCGTCGCCTCCACTTGAGGACCCGCCTGTCCTTTCTTCCC
CCGACCCCTCCACCTCTGTGTTCTTCAGTCTCCCCGCATCCTCTGCCCTCGGTCTTCCCCTCCCCAG
CTTGCATTTTTTCCCATCTCTTAGTCTGCCTTTCATTTTCCCCTTCACATCCCGGTGGCTCCCCTTCC
CCGCCCCGTCCCTCTGCACACCCCCATTCATCTCTGTCAGGTTTTTCTCATCCTTAAGTTCTCTCGT
CCCCCTCCCCACTCGGGCGTCCCCCGCTGGGCTCCCCGCCTCAGTTTCCTTCCCTTCCTGACTCCTT
CCTCTGACGGCGGCCCTTTCTCGAGGGCGGATGTGCGCCTGGAGGGCGAAGGCGGCGGCCGAGA
GGACCCCAGCTCGGCCTGGCGGGCCTCTGGCCACAGCCATGCACCGCTTGGGCCGAGGCCGAGG
CCGACCCCCAGGGACACAGGTGAGGCCGGGCGACCCGCGGGGCTCCGGGGAGCGCCCGAGCAGT
GGCCCCGCGAAGGGCCGGGTCTGGTGCTCTGGGGCTGGCTTTGGACCTCTCGTCCTGGGACTCCG
TGGGAGCCCGGGAGCTGTGTCAGTGCCCGTTGGTGCTGGAGCCAGTCGGCCCTTCCTTGGCCTCC
TTCAGGCGGGGGCCAAGACCCCCGAGAGTTCACTCTGGGTGCTCCACTCGGCGGAGAGGGCTTCA
GACTGGTGTCCCGCGTGGCTGGGGTGTCGGCGCATTCCCGCGGGGGAGGAGGCCGAGGGCCCGG
GCCGGGGTCACCTGCCGGCGCCCCGGCCTTGCGCACAGAGGGCCTTGCCTGTCGCTCGTCAAATC
CAGACACCTGGGCCTCCGCCACTCGGCTGCCGGGGCGGCACTGGGGCGTCTGTACCCAGCGTGCG
GGAGGGCACCCAGGGCCGGACGCGCACCGCAGGGGTCTTTTTTTGGCCGCGGGGCCGGGCGGGC
GGGTAATGACAGCCGGGTTGCTGGGGAGCCGCGGGCCAGATGGAAGCGGGGCCGCAGGTGAGG
GACGTCCCTCGCGTAGCGCCACTCAGCCGCCGGGGCCAGAGCGGGAGTCAAGGTGAGGGCGCCC
CCAGGTGGCCGCTCTCGGCCGCGGCGATCG
CGI: 97 range = chr12: 1844001-1845219
CGTGCGGGTGACTCCTCTTCCTGCCGACAAGGTGGGCGATGAGAACGAGGAGGCCCAGTCCCAC
CAGGGCGCCGCCGACGGCGATGGGGATCGGCGTGTTGTTCTCGCCCAGCAGACGCTCCTCCGCAG
ATCCGAACTGGTTGCCTTCCACCTTGAAAGCCTGGCCCCACACTGGGAATATGTTGATTGAAAAC
GCCTTCGTGCCGCGGACACGCTCCGCTCCGGCTGTAGAATTGCCGATGGCTGCCCGCAATGCTCG
CAGGGAGTCGTTGGCAGCTTTAAAAGTGGGGTCTCTGGGGTCAGGAAGAGTCGTATTCAGCTGGA
TTCCTCGTAGGAAAATCCGGCTAGAACCTGCATTCATCCCCAACCGGAAGCCCAGGACCGTGGTG
CCCTCGCCGCGCCGCTCCCGGGTCTCCAGGTGGGCGCCGCGCTCCCGCTGGCTGAGGTCATGCCC
AGGTCACCGTCGCGTGGTCCCTCCTCTCTGGGTGAGGTTCAGCTGCAGTCCCGCGCTGGCCGGCA
GGCAGGCCCCGTGGGTGCCGCTCACGCTGTACCTGGCCACGGAGGGGAGAAGGGGCTCTCGGCG
CCGTGGCGGGGAAGGCGCGTCTTGCTCGTGGCGCGTCTCTCCCTTGCTGAAGCTGCTGTTGGAAA
TCTAGGCCTGGATCGGGGCACCGCGGAGCCTGATGGTCTCGCTGTTCGTGCGGACCTGGGGGCCT
TGATATCTGGTAGAGATTCCACAGTCCTGATTTCCTTGGAGCTTGCACTGAGGAAAATGTGTGTGT
CTGACAAGTTGTAAACAAAACTCATGAGCTGGACGCTGTCACGTGTCGCACTTCTTGTGAAATGG
AAGGTCAGTGTCTGTCTTCCTCCAAAGCACAATCGCGTGCCTGGGGTCGGAAGTGTTCTCTTTACT
GCAAGAGCCACTGTCAAGCACTTCCGCATTGGATGGCAGGTCAAAGGTCACATTCTTAGAGCCAC
TCTTAGAGTCGTAGTTCATCGAGAAGGCAGCGAAGCTGGCCATTACGCAGGCTGTCCCGTTGCCC
TTTTTCACCGCATCCGCTGCGGCTGCACCACGCACGAGGCCCAGCAGCAGCAGCAGCACCAGGG
GCAGCCGGGGGCTCCACCAGATGCAGCCCGGGCGGCAGGCGCGAGGGGAGCCGGTGTGTGGGG
AACCGGGGCGCGGGGAGGCGGTTGCGGGGCGCTGGCCTGAAGGACGGTGGCG
CGI: 16 range = chr12: 40253533-40253742
CGACGGGACACGGTACATCACAAAGAGACCCGTGCGAGACCGAATCCTGAAGGAACGTGCCTTA
AAGATCAAGGAAGAGCGGAGTGGCATGACCACAGACGATGACACCATGAGCGAGATGAAAATG
GGGCGCTACTGGAGCAAAGAGGAGAGAAAGCAGCACCTGGTTAGGGCCAAAGAGCAGCGCCGT
CGCCGTGAGTTCATGATGCG
CGI: 79 range-chr12: 52726910-52727810
CGTGTTCCCGGGCGGAGGTGCGCGCAGCCACCCCAGGCTGCTGCCAGGTGCCCGCTGGGGCTGCC
AGGGCGAGGAGGCCTCTGGGCTGTGGAGCGAAAGTCAGATCCACCGCCTACTGCGGGGTAGGGG
CCGCAGTGGGGACCGCCAGCCCTGTGGTCCCTCTCGCGCTGACTGGCGTAAAGTTGTGGCCGAAT
TCGCATCTCTTCTGGTGCTTCTCGCCCGCCAGCGCAGGGCCCAGGTGTTTGAGGCGAAGGGGCTC
TAGCTCCCCGCAAGCCTGGAGCCAGGCGTCGCGCTTCCTCCGGGCTTAATCCAGACCTTTCAACA
CACACCTCATTCGGGGGAGGAGAAAAGCACAGGACCGCGGAGAGCCCAGCTTTGAGGCCAGGCC
TGAAGGGATAACCCACACAGGGAACGTTTTCCTATCAGAGAATAATGGAGCACAAAATAATTCA
GAAAGCGAATGGGCAGGACCACAGCCTGAGAGTCCCGCGCCGCGGGGCCGCTGCAGAGCCGGTC
TCCCGAGCACCGCGGCAGGACCATTTCGTTGGAATGTAGGGCGAGGCCGAAGCCCGCCCCGGAC
CCAGGCCGCGAGGTGCGCGCCGGCCGCCGAGGGGCCGCCTGTAAATTACAGCCCGCCGGGAGGA
CTCGGAAATACACAAAAGGAGCCGAAAGATTTAAACAGTCGGAGGCAGAGGCGTCCCGAGGCG
GCCAAAGCGGAAATCAATCACGTAATTAAAACAGGGAGGGGACGAAGCCCAAGGCTGGGGGTC
CCGGGTTCGGAGGAGGCGGCCAAGGTGCAGGCCGAGGCTGGCGAGCGGCTTAGGGACGTGGCTC
GCCCGCCAGGACCAGAGCGCGCGGAGGGGCTTCGGGGAAGTTTATAACACATCGCTATTGATTCC
CG
CGI: 32 range = chr12: 79634857-79635257
CGGAGCGACAGACTAGGGAGCTCCGCCCGGGATTTGCCCATCGGCGGAGGCGCCAGGCTCCCGT
TTCTCCCCATCCCTCTCGCTGCCGTCCAGGTGCACCGCCTGCCTCTCAGCAGGATGGACGTGATGG
ATGGCTGCCAGTTCTCACCTTCTGAGTACTICTACGACGGCTCCTGCATACCGTCCCCCGAGGGTG
AATTTGGGGACGAGTTTGTGCCGCGAGTGGCTGCCTTCGGAGCGCACAAAGCAGAGCTGCAGGG
CTCAGATGAGGACGAGCACGTGCGAGCGCCTACCGGCCACCACCAGGCTGGTCACTGCCTCATGT
GGGCCTGCAAAGCCTGCAAGAGGAAGTCCACCACCATGGATCGGCGGAAGGCAGCCACTATGCG
CGAGCGGAGGCG
CGI: 30 range = chr12: 95164929-95165298
CGCGGTGGAGATCAGCCGGGAGAGCCTTCTGCTGCAGGACAGCGACTGCAAGGCGTCTCCGGAG
GGCCGCGAGGCCCACAAACACGGCCTGGCCGCCCTCAGAAGCACGAGCCGCAACGAATACATCC
ACTCAGGCCTGTACTCGTCCTTCACCATTAATTCCCTGCAGAACCCACCAGACGCCTTCAAGGCC
ATCAAGACGGAGAAGCTGGAGGAGCCGCCCGAAGACAGCCCCCCCGTGGAAGAAGTCAGGACT
GTGATCAGGTTTGTGACCAATAAAACCGACAAGCACGTCACCAGGCCGGTGGTGTCCCTGCCTTC
CACGTCAGAGGCTGCGGCGGCGTCCGCCTTCCTGGCCTCGTCCGTCTCG
CGI: 35 range = chr12: 113609113-113609535
CGCGCGAAGCAGAGAACGAGAGGAAAGTTTGCGGGGTTCGAATCGAAAATGTCGACATCTTGCT
AATGGTCTGCAAACTTCCGCCAATTATGACTGACCTCCCAGACTCGGCCCCAGGAGGCTCGTATT
AGGCAGGGAGGCCGCCGTAATTCTGGGATCAAAAGCGGGAAGGTGCGAACTCCTCTTTGTCTCTG
CGTGCCCGGCGCGCCCCCCTCCCGGTGGGTGATAAACCCACTCTGGCGCCGGCCATGCGCTGGGT
GATTAATTTGCGAACAAACAAAAGCGGCCTGGTGGCCACTGCATTCGGGTTAAACATTGGCCAGC
GTGTTCCGAAGGCTTGTGCTGGGCCTGGCCTCCAGGAGAACCCACGAGGCCAGCGCTCCCCGGAC
CCCGGCATTAGGCGCCAGCTGCCGGCTATCTGCG
CGI: 121 range = chr13: 35817738-35819004
CGCAATAATGCTGCGTCACGAAACTGCGCATTCCAAGAGAAACTTTTCTCAGGCCCAGAATTAAG
TCGGAGGGATCCGCCGCGCAGCTCAATCCCTAGGAAGCATGAAAGAATGTATTGTAAAGAGTAA
AATCACGCGCAATCCATCCCAATATAACCGCAGTTGTTCGTGGGCCTTCTTGCAGAGAAAGGCTG
CCCAACCTTGACGGGGCCATGGGCGCTGCGGGAGGGCAAAAGACCACAGAGATCTGCGAAGCCA
AAGTAAACAACGGGGTTGGGGCGGCGAGAATGATCAATAGCGATGCTCCGAAAGGACTCCGCGA
TAGGAATCGAGCGGGAAGGATTCCCTCCATCCTAACAGTCCATGGGTTAAGAGGCCGAAGCTTCC
CTAAATACCACACTCCCGCGAGAGAAGGGACTGAGAACAACTTCTCAAACTATTCTCTCTCGAAA
TCGCTCCCTTCCTCGAAAATTCCATCTCTGAGACTCGGATGAGGTCCCCACCCCCTCCACCCTTGT
CCCGTGACCGTCGCCCGCTCAGCCTCCCAGCCGAGTCCGCGGGGCCTGGGGCGCCCACCCCGCCC
ACCCAAGGGACCGCGCAGGGGTGAACTCCCCCGCGCCCCACCTGCGCCTTCCAGACCTCGGGCGC
CCCGGCGTTGCCTCGAGAGCTCCCTGCGCGGCCGCCGCGGCACGGACCAGCTCCCACTCCCTTAC
ACTGGGCGCCGCTGCGCTCGCCGGGGGCCGGTCCCGAGGTTCCCAAGGCCTCGCGCGCGCGCTTG
CCGTGGCAACCAAGACGTTCCACGACGCGCGCTCTCGAACGCTTCGCGTCACGCGGCCGCGCGGC
CCCGCCCGTCGGCCTCGCTCCCGCCACAGAGCCCGCAGCACGCCGCCGCCGCAGCCTAGGTCACG
TGAGTACCCACGCGCGCGTCTTGCCAGCGGATTCATCACCGGCCTGCTCAGACTAGGTTCTGCCC
ACTCTGACCTTCTAAATGGTACGTGGGAGGACGTCCGTCCCCTTCGGACCCAAGAGTCACCGTAA
CACTCTAGAAGGGGAGAAAAGGAGCGAGGGCGGCAGGCGACAGAGAACCTCGCGAGTCAGCGG
CCCCGCGCAGACCCCCCCAGGCACGGTCCCCTGCGGCCACGTCGGCTGCTCGGCGCCTGCGCAAT
CTCTTTCTCTCCAGCGAAACCGAGGCCTCCGGAGAGCCTAGTAGAGAGTGTGGGCAGTGAGCGCT
TGTAGCCGCTAGAGGGAGCGCTGGGCACAGTGCACG
CGI: 58 range = chr13: 57101588-57102323
CGCGGCATGTTCGTACCAGCGCGGGCAGAAACGGGCTCACGAGCGCCCTCGAATTCACACACAC
TCACCGAAATACACATCTCAGCCCGTGGAAGCGCATTCACACACACCCACGTACACTCCTCCGCA
CCTCATTCAGATGGGTCCACGCGGAGTCAGGGCTGCGCTTCCCTTGGCATGGGCGTGTGTGCGGC
GAGGGCGTGTGTGCGTGCGTGTGTGAGAGTGTGTGTAGAGAAACCCTTTAGGCAGACCGCAGCT
ACGTTATTCTGCAAGCATACTGCGATTCCCGATTCCGTGTATGTAGGACCACAAAAAAATTAAAT
CGCTGTTAAACTTTTTTTTGTAAATTCTCTTTTCCGAAGGAAGGCGCTGGAAGAGCTACTCCCGGT
GGTTAAAGGCCCGGGAGACCTGGTGGCGAATCCTGGGGCGCGTCCGCTTTGGCGGAGCGCTGGG
CTCCGCTAGCCGGACTGGTTCCCCATCTAACTGATCTAATCAATGACTCCGAAGAGCCGGGGCTC
CTGGCCCCGCCCCCGCCATCCTCTTCTCCGCCTCCCATTGGACGCGGGCCGAGAAGGCAGGGCCT
TGCTCCTCGCTCGGATTGGCTGACAGGGAATCAGTATCAATGTTTAAGCGGCGGCGTGAGGTGAA
TAGAGTCAGTCAGCAAGAGACGCTGGGGAGAGAGCAGGGATCGCGAGCCCGGCGGATGCGGCA
GCGGCGGAAGGCGGCTGGGGAGACG
CGI: 30 range = chr13: 105943410-105943788
CGCCGGGCTCTGCGGGGGCATCTCCTGGACGATGTACACCGGGTGCCCGTAGTCGCCGCTGACCT
TCTCGTAGTGAGGGCAGAAGACGCTGTCCGCAGTCCTTAGCGGGATGATAATGTCACTGGGCTCT
GAGCCGTTGTTGTTGCCGCTGCGCTTGGGTGTGGCCAGTGTGCTGAGCGACAGCGTGGTCGTGTG
CTGCGGCGAGTGCTTCCTGTGTCTCCTCCGGTACTTCAGCAAGAGGACCACCAGCGTGATGATGA
TGACGATGAAGATGATGCATCCTGAAGCAATCCCTGCAAATAAGGCCACTTCGGAACCGAGGAT
GTTGTTCCCCGAATGTCCGGCGCTGTTGCCGTCTGTGCTAGAACCTGCAGACGCG
CGI: 74 range = chr13: 111763361-111764235
CGCTCCCAGCCGGCAGCCGCTCTGGGCGTGCACCCGCCACGCGAAGCCAGAGGTTGGACGACGC
AGCCGGCCAGGCGCGGCTCCCGGGGGACCCCGAAACCGCCTGTGGCTCAGGCCCTACTTGGCAA
GAAGAGACTGGGGAGTGAGAGGAGGGGTCACTCTTGCTCTCTCGCACAATAGACTTCCCGCAGG
CGAGTGCGAACGGCCTTTTGCTTCCTCCTTCTTTTCCAGGACCCTTAGCGACAGGAGAGGAAGAG
AAGCGGCCCGGCGGTTTAATTGCCAATTTCCTGATGCAGCCACAGGGCCCAGCAGCGGCTCAGCC
TAGGCCACCGGGTCCAGGCCTCTAGGCAATAGGCCCGGCGCCCCCTCCAAGCACCGTTTCCCGCC
GACCCCGCAGTCATCACTGTGAGCTCGAGAGTATGGGCAGCCTGTGGTGCCCTGAGGCGCCGGGC
GCTCGGCGCATCCGCCGTGCCCACAGCGCTGAGGACCAACTAGGGCTGCCCTCGGCCAGGGGCA
CTGTGCCCGTTTGAGTGGCACTCGGACCTGGCGAGGGGTGGAAGCCCAGGTAGAGCTCGCGTCTT
TAAGAGCCACCCGACAGAACTGCGCGCTACATCTGCGCCAGCTGGGCTGGGTGGGAACAGCGCA
GAAATACTCCCGTGCCGGGGCGTACAGGGCAGGTAGGCCCAGCAGAGAGTCTTCTCGGAAAGTT
TCCCACAAAAGAAGGGCCGGGCAGGCCGAGCGGCAGGAAAAGCCGGTGCTCAGCGGCGAGGCC
CGAGAGGTCTGGCATCCGGCGGCCTCCCAGCTTTGCTCGGGTCTCCCGCCGCCTCTCTCCAGAGC
CGCCCGTAGCTCCGTGGGGCGGAGGGCGCAATGCCCCG
CGI: 28 range = chr13: 112249708-112249928
CGATTCCTATCGAATCAACACGGCCATTAAGGACAAAAACGGGCAGGACACAAAAGCAAGCCTG
GATGGCGCACCCTACAAACGAGTTCTGAAACAAAGCGAGTGCCAGACGCGCGTGGGAAAGACGC
GCGTGGGAAAGACGTGCATGGGAAAGTCGCGCGTGGGAAAGTCGCGCGTGGGAAAGTCGCGCGT
GGGAAAGACGTGCGTGGGAAAGACGCGCG
CGI: 87 range = chr14: 19992581-19993863
CGTGACGTAAGTCCGCCGCGGGTTCGCCAGCACCTTGCCATCCCGCACCACGCCCACGCCAATCT
TATTGGCGCTGCCTTCAAAACCCAGCACCGCCGGCATGGCGGAGGCTGGGAGAAAACGCCGACA
GGACTCCTGGCAATGTCAGGAGCTGTGGAGGTCCTCACTAGTCCGCGCTGGGCCGCAGCTTTCCG
GAGCGCAGAGGAAGCTGGCCAGCCTGCAGATAGCACTGGGAAAGACACCGCGGAACTCCCGCGA
GCGGAGACCCGCCAAGGCCCCTCCAGGGACCTGTCTTCCTAACTGCCAGGGACGCCGAGCCAACT
CTGTGCCTTACATTCGTATCCGTTTTCCTATCTCTTTCCCGTGGTCCAGCCCAGCCTTCTCCACTGT
TTTTTTCCCTCTTGCACATAGTTAGAATCTTAAGTCAGTGTCACACAATGTGCTGTGCATCTGGCA
CAACGATAAACAGCCCGAGGGAGGGTTGGGGACCTAAGTGTCCTAGAGAATTAGAGGAGGGAG
GCGAGGCTAAGCGTCTCCGTCACGTGGTGTCAGACAGACCAATCACGCGCATTCTTCGGCCACGA
CAAGCGCGCCTCTGATCACGTGACCAGGTCCGCTACCCACGTGGGGGCTCAGCGTGCACCCTTCT
TTGTGCTCGGGTTAGGAGGAGCTAGGCTGCCATCGGGCCGGTGCAGATACGGGGTTGCTCTTTTG
CTCATAAGAGGGGCTTCGCTGGCAGTCTGAACGGCAAGCTTGAGTCAGGACCCTTAATTAAGATC
CTCAATTGGCTGGAGGGCAGATCTCGCGAGTAGGGTACAAGGCACTATGAAATGATCTAGTTTCG
TGGGTGAGGGGCTGAAGGGCCTATGATGCACGGAGGCGGGGAAAGGATTTAGAGATAACGTGGT
TTGAAAGGCGGGACCTGGTGCGGGGACGCTCTTGGGAGGAGTCTTCTCCCCAGCCTTAGCTGGTT
TCATGATTTCTTTGCGTCTGTAGGCAACGCGGTAAAAATATTGCTTCGGTGGGTGACGCGGTACA
GCTGCCCAAGGGCGTTCGTAACGGGAATGCCGAAGCGTGGGAAAAAGGGAGCGGTGGCGGAAG
ACGGGGATGAGCTCAGGACAGGTAAGGGAATGAAATCAGCCCTTCTTCCTAGAAGCTGCGGCGG
GGGTGTTTGTCATTCCCTTGATGTACGGTAAGTACGGGCCGACTCATTTTTGCAGGGGTTTGTGAA
GAAGTCGCAGGAACCGTAGGCTTTCGTTGGGTCTATAGTTAACGCCGGATCG
CGI: 79 range = chr14: 23710894-23712060
CGCCTGTTACTAGAGGCGAGAACCGGAGCCCATTGGTCGGAACACCTCACAATGGACCCCAGCG
GCGCGCAAAATCCTTATGATTGGTTTGCTGGCTGCCTCGGGAGACCCTGTTGCCAGGATACTTGG
CGTTCCCGACCCGACCCCCGTTCCCCATTGGCTGTCAGGGCAAAAGCCGCCATCTAATGAGGAGC
GAGGTGCGGTGCCCCGAAGCGCTCGCTTCCCGCGGTGCGATCTAGTCCTGCAGTAGGCGGCCCGG
GGCCACACCGCGGCCGCCCAAGCCAGTGCAAGGCCCAGGGGCCTGACATCGCTCCCAGCGCTCG
AGGACCGAGGCCTGCTGTGGAGGACACCGTGCTCCCTCGGGACCTGCTCTGGATTCCGGCCCGGA
CGTCCCCTTGGAGCTCTGCATCTCCAACCTGGAACCCAACCCAGAAGTCTCAAGTTTGACGCATC
ACGTGGCGTGCGGATCCACTGAGGGTCCACAGAGAGGGGCGCCCATCTCCTGCGTCTCAGTTATC
CTGGTAATTGTGTATCTGCCCATTGTTCGTTGCCTCATTAACTTGGCTTTCTAGGTGCACCCACCTT
GCCACCAGAGAAGTCCAAATCCTGACTTCTCTCCAAGGTGTTGGGAATTCTGTGCCCTAAAGAAT
TCCGACTCAGATCCGAACGGGGATCTGGTGGAATCGAGGGTGAAAGACCAGAGGGACAATGTTC
TACTATCCCAACGTGCTTCAGCGCCACACCGGCTGCTTTGCCACCATCTGGTAAGGGCGGGGCCC
GTTGGCGCGCGATGGCGGACGCTGCCCGGGATCCCAGCCTGACAGCTCCCCCTCCATCCCCATTC
TCCCACCTTCCCCACCCACTTCAGGCTGGCGGCGACTCGCGGCAGCCGGTTGGTGAAGCGCGAAT
ACCTGAGGGTGAATGTGGTGAAAACCTGGTAAGGCCCAGAAAAGGGAAGGAGGGCCTGGTGCG
GGGGGTGAGTTAGGGGATGGGGTGGCCAAGACTGTGGGCCCACTCCTGGACGCAGCGGTAATCA
GGGCGCATTGTTCCCCAGCGAGGAAATCCTCAATTACGTGCTGGTACGAGTGCAACCCCCGCAGC
CCGGCCTGCCGCGGCCCCGCTTCTCCCTCTATCTCTCAGCCCAACTTCAGATCGGTGTGATCCGCG
CGI: 23 range = chr14: 28317076-28317375
CGTAGCGACGACGTGGTCCTTGTAGGACCAAGCATTCTTGGCATTCTGTCGAGATTGAGATTCCA
GAGCATCGGATGAACGGGACTTATAGCTAAGGCGTACTCACCCACCCCTCCCTTCGTCTTTTTACG
CACGCAGCAACTCGTCCTCACGTGCATAGGGGTAGTGGACAGTGTTGACTTGAATTGCTGTCCCT
CGTTCCAAGCCTTTCCTGTGAATGAACCCGAAAACACTCGACAGGTCGTGAATAATCGTTTTAAT
GAGTGTGCAAAGCGTGCGACGGGGTGCACCGAGCTGTCG
CGI: 147 range = chr14: 33338698-33340189
CGAGAACTCCAAGTCCGACGAGAAGGGGAACCAGTCCGAGAACAGCGAAGACCCGGAGCCCGA
CCGGAAGAAGTCGGGCAACGCGTGTGACAACGACATGAACTGCAACGACGACGGCCACAGCTCC
AGTAACCCGGACAGCCGCGACAGCGACGACAGCTTCGAGCACTCGGACTTTGAGAACCCCAAGG
CGGGCGAGGACGGCTTCGGTGCTCTGGGCGCGATGCAGATCAAGGTGGAGCGCTACGTGGAGAG
CGAGTCGGACCTGCGGCTGCAGAACTGCGAGTCACTCACGTCCGACAGCGCCAAGGACTCGGAC
AGCGCAGGCGAGGCGGGCGCGCAGGCCTCCAGCAAGCACCAGAAGCGCAAGAAAAGGCGGAAA
CGGCAAAAGGGCGGCAGCGCCAGCCGCCGGCGCCTGTCCAGCGCGTCGAGCCCAGGCGGCCTGG
ACGCGGGCCTGGTGGAGCCCCCGCGGCTGCTGTCCTCCCCCAACAGTGCCTCGGTGCTCAAGATC
AAGACGGAGATCTCAGAACCCATCAATTTCGACAATGACAGCAGCATCTGGAACTACCCGCCCA
ACCGGGAGATCTCCAGGAACGAGTCCCCCTACAGCATGACCAAGCCCCCCAGCTCTGAGCACTTC
CCGTCCCCGCAGGGCGGCGGCGGTGGGGGTGGCGGTGGCGGGGGGCTGCACGTGGCCATTCCCG
ACTCGGTCCTCACCCCGCCCGGCGCCGACGGCGCGGCCGCCCGCAAGACTCAGTTCGGCGCCTCG
GCCACCGCGGCCCTGGCCCCCGTCGCCTCCGACCCGCTGTCACCCCCGCTCTCGGCGTCCCCGCG
GGACAAGCACCCCGGGAACGGCGGCGGGGGCGGGGGCGGGGGCGGCGGCGCGGGGGGCGGCGG
CCCCAGCGCGTCCAACTCCTTGCTGTACACTGGGGACCTGGAGGCGCTGCAGAGGTTGCAGGCGG
GCAACGTCGTGCTCCCGCTGGTGCACAGGGTGACCGGGACCCTGGCCGCCACCAGCACGGCCGC
GCAGAGGGTCTACACCACGGGCACCATCCGCTACGCGCCCGCCGAGGTGACCCTGGCCATGCAG
AGCAACCTGCTGCCCAACGCGCACGCTGTTAACTTCGTGGACGTTAACAGCCCCGGCTTTGGCCT
CGACCCCAAGACGCCCATGGAGATGCTCTACCACCACGTGCACCGGCTCAACATGTCAGGACCGT
TCGGCGGCGCAGTGAGCGCAGCTAGCCTGACGCAGATGCCCGCCGGCAACGTGTTCACCACGGC
CGAGGGACTCTTCTCCACGCTGCCCTTCCCCGTCTACAGCAACGGCATCCACGCGGCACAGACTC
TGGAGCGCAAGGAGGACTGAGGCGCCGCCCGTCCTGGGCCCGGCCAGGCCCCGCTTGGAGGAGG
CATCGTCGGCATTTTCGTTTAGACCTTTAATTCTAGCACTTTGAATTCGAGCAGGTCAGCGTCTTC
TCTCGCCACGACG
CGI: 22 range = chr14: 36122289-36122589
CGCCCGGCCGCTGCCGATCTCGGAGCTCGGAGGCCGAGCTCCACAAATTTGGGTCCAGGCCCGCT
TTAGAGCCCCTGGGCGGGGGCGCTTCTCAGGGCATCCTGTGAGGGCTTTGAGGCCTCCTTGGAGT
CGCCAGATGAACTGGTGCCGCCTGTGCGCCTTGGCCCCATAGCCTCAACGGCACTCTCACTGCCT
GGGCTTTGGTCTTTGCCCAGGAGCCTGCCCATGGCCTACGCTCCCCGCTCGCAGCATGGCAGACC
CTGACGAGGCTGCCCGCCTTGGTCCGGGAATGGACCGATCG
CGI: 123 range = chr14: 37137198-37138958
CGGGGCAGGGTACCCGCGAGGGCAGGCACGTGGCGGCTCCTGCGGTGTCCCGGGTTAAACTTGC
CTGTGTTTAAGACGGGTCTGCGACAGCTTGGGGCGGTCCAGGTCGCGGGGAGGGCGGGCTCCTCA
AGTGGGGGATCCGCGGCAGTGAGGACTGTAGGGTGCGCGGGTACTCCGGGTAGCCGCCAGCGGA
GGAAGCGGCTCCGCCCTTCCCGGAAGCTGCCCTTGCTACACCCGTGGGGCCTGCTCCCGGCGCAG
TCCCGCAGTACCTGAGCGGCACTCGGTCCTTTGACCACGCTTGGCTGCCTCAGCTTACACTTCCCA
GCAGGATCTGGCTGGGAGCCCCTTCCTCAGATATTCACCAGGCAGGTAGAAGCAGAGGGAGACG
ACGGCGAAAGGTGGAAGGAGAGGCCGCGGCCTCGGGCCTCGGGCCCCGGGCCCAGACGGCGGC
CTCTCGCGGCGACAGGGTCCTTCCTATTTAGCAGGACCGCAGGGTTGGCAGACAGCAGGGCCTGG
GCCGGCGGGGCTCTGCGGTGCCCATTCGAGTCTCTCCAGGGCTTCCCTCTGTGGCTGTCCCTCGCC
TGCCCCCTTTCCTTGCCTACCCTTCCCTGGACGGGGTTGTTGTCAGGTGACGGCTTCAGCAACCCA
GCCATGCTTGGTGTGGAGTGGTTCGAGTTCCTCCGCGGGCCCCCACCCCCCACCCAGATAAGAAG
CTTAAGTCCTCCTTCTGCAGGGGAGAATCGGATGCAATGAAACTAAGACCTGTGGTCGTTAACGC
GGTTCAAAGCCGACCCCAACTGGGACACGGCTCAGCTTAGAAGTGAGGAAGGCCTGGGACGCTG
GAAACAAGGGGTCACACGTGCGTGTGCCTGGCCTTCGCAGGTGGATGTGTGCGCCGAGCTGGGG
AAGGGGCCAGGGAGCCGCGAGACTGCGGTCCCCCGCGAGAGAGAAGTATGGGCCTGGCCAGGGT
TCGAACTCCGTTCCTTCGCTGCCTTCCTTTTATTTTTCTTCCTCATCTTCCTTCCTCTCTCCCTTGGT
TCCATCATTAAATCTTTAAGGGATTAATTCACAACTCTGTAGTAGGTAGGGGCCCTGGGACCACT
TGACGGGCCTTTGCAGGCAGTTCTGTACAGAGTCCTAACCCGTAACGGGCCTCAACCCCGGCTGT
GTGTGCGTGTGCTTGTGTCTGTGTGTCTTTGTGTGTAAAAGCAACGCATAAACATACCCAGGGCTT
TCACTTATTTTATTGAAGCAAACAAACAAAAACAGCCACTGGGTTTGTCTTTTCCCCTACTCCTGC
CTGAGCGCCGCCGGGTTTCTGAGCTGCTCCGGGCGCGGACCTAGCTCACCCGCAGCCGGATGTCG
GAGCCGGAGCTGCTCCCCACCCGGCCTCGCCCGCGGGCGACAAAACCGTCCCTCTGGCCACGGG
AGAGCATCGCCTTGCCAAAATCAACTCGCATTTTCATGTATTCAAATGAGAAAGACTGAGAAGGG
CGATTTCCCCTGCTGGGCAGTCGACAGCGTTGGAAGGAAGAAGTGCCCCCTAGGGCCTCGCGGA
AACGGGAACAAACCTCCCTCGAGGCCCCCGGAACCGATATGGCGACCCCGCCCAGACCTGGGTC
CCCGCGGCCGCGCGCGTCTCCCGCCGTTGTGGCCCCCGAGTTACTGACTTTTGTTCACACCGGTCA
GCGCCTGTTGGAGGACGTGCTCAGCCCCAGCCGGTCCCTCCTCATCCAGCCGATCCCCCTCCTGG
GGAGCGCG
CGI: 20 range = chr14: 56347822-56348040
CGGGCCTCGAGGATGCGAGTGTGAGGTCGACAAGATATCGTGGAAGAGAGCGAACGAGGGAGG
CCCCCGTTGGAAACAGTTTCTGTGTTAGAACAAACGCCACCAAACGACCGACACTGCGAGGCCTG
GAGCTACCTGGGCTGGCGCCGCCCGAGCCCCGGGCTGCGAGCCCAGAGGCTGGGAAGTGATGGG
ACCTGGGGGCCCGAGCCGGAGAGTGCG
CGI: 178 range-chr14: 60045486-60047933
CGGCGGGCGCTGTCGAGCACGGGGAGGTGCTGAAATAGTCCTGGCGTGCTGATTCAAGCTTTGAT
TGGCAGAGCCACCCGGTGACTGACAGGGGGTCTCCATGGCGCCCGCGCCGCCAATCCGCCCACCC
CAATAGCGGAGCCAGCTCGCCTGCCGGCGTGCCTGAGCCGAGCCGAGCCCGAACCCCAAGCCGC
GGAGCCAGCACCTCCTCCAGTCGGGGTCGTCCGCTCCCGGCCGTTGAGCCACCGCCGCCACCCGG
TAGTGTGTCCCGCTGCCCCAATCCGCCTCATCAACAAGCGCCTGGCACACTCAGCCAGGCCCGCG
GGCATCTGCTGCGTGTCCCGCTCCGGGCTCAGTGCCCTCGCCGCCGCCGGCACTGCCTCGATGTTC
CAGCTGCCCATCTTGAATTTCAGCCCCCAGCAAGTGGCCGGGGTATGTGAGACCCTGGAAGAGAG
CGGCGATGTGGAGCGCCTGGGTCGCTTCCTCTGGTCGCTGCCCGTGGCCCCTGCGGCCTGCGAGG
CCCTCAACAAGAATGAGTCGGTGCTACGCGCACGAGCCATCGTGGCCTTTCACGGTGGCAACTAC
CGCGAGCTCTATCATATCCTGGAAAACCACAAGTTCACCAAGGAGTCGCACGCCAAGCTGCAGG
CGCTGTGGCTTGAAGCACACTACCAGGAGGCTGAGAAGCTGCGTGGAAGACCCCTGGGACCTGT
GGACAAGTACCGAGTAAGGAAGAAGTTCCCGCTGCCGCGCACCATTTGGGACGGCGAACAGAAG
ACACACTGCTTCAAGGAGCGCACGCGGCACCTGCTACGCGAGTGGTACCTGCAGGATCCATACCC
TAACCCCAGCAAAAAACGTGAGCTCGCCCAGGCAACCGGACTGACCCCTACGCAGGTGGGCAAC
TGGTTCAAAAACCGCCGACAAAGGGACCGAGCGGCTGCAGCCAAGAACAGGTCGGTACCTAGAG
GCCTCCGCGCTTTGAGCGCACCGGGGAGGAGGCGGGTGGAGGCACCTCTGGCGCCCTTACCCAGT
CCCTGGCGACTCCAATTCAGCAGGAGTTGGGAGCGCGGTCTGTCTTGGGTTAAGAGCCCTGCGTT
CTGGGCTCCTGGCCGGGAGTTCCCTTGCCGGCTCTGCTTCCCCACCCGCTGGCTCCCCACGCCTGC
GGGCAGCTGCAGCAGCTGGTCCCGGTCACCAAACCAAGGCTTCACTGGGACGGAGAGGGGAAGA
GAAATAAAAAATTAAAATCCTACAAACAGTTAGGGACCCCAAGACCCAAAGCTAATTCTTGTCA
GCCTGGGCACAGGCTCCTACTATTAATCGAAGCCTGGCTTATTAGCAATGTGTCGGTTTCATGTTA
ATTATCATTTTCAAAGCCCAGGTATATCCCTCCCTAATGCTTTGAAAACAGTTTTCAATGGACTTT
TGAGAAATGGGAAGTCGAGTTTTCCTCTTCCCATGCGCTGCCTGCCACTCTTGTCTCAAAACAGCA
AACTAGTCCGTGGGCCGAGGCTTTTCGTTTCCCGGAGTGTGGATCTCGATTAGCCAAACATTTTGC
GGAAGAGCCCGGCCTCATCCCCCAGGCCCAAATGCTCCTTACAATCCTTTTTGCCTTTAGGTCGGG
CCGACCCGATCCAACGCGATCGCGGGAGCACTTGCTCAGGCGTAAGCCCCAGGCAGACGCACCG
TTAGAAATGGTATCCCATGTCCCTGGGACCGATCTGTCCTTGTCACCCACACTTCGTTTATTTCCT
GACAGTCCTGTAAATCTCCCAAAAGTGCACAACAAACAGGGAGGACACTGCAAGCCCAGTATAT
AAAAGACCTGGGAGCTGCGGCGCTGAGAAAGGGCGCGAATCATGGTGGGGCACAACAGTAGGG
ACCCGCGGAGGGGCGGCCGCGGACTCCTGCCCGACCTCTGTCGCCTTGCCGAGTAATCCTCGCCT
TAACTGCTGGGGTCTTCGGAAGAACCTCTAGCCGCCGGGCTGGAGGGACGCAGGAGGTGGTGGG
GGCGGGCGACGGGCGGCTGTGTTACGAGCTGTGACCCGTGTTCCCTTTCTTCCCCGTAGACTCCA
GCAGCAGGTCCTGTCACAGGGTTCCGGGCGGGCACTACGGGCGGAGGGCGACGGCACGCCAGAG
GTGCTGGGCGTCGCCACCAGCCCGGCCGCCAGTCTATCCAGCAAGGCGGCCACTTCAGCCATCTC
CATCACGTCCAGCGACAGCGAGTGCGACATCTGAGTTGCCCATCCAGGATGCTCAGAAGCAGATT
CCAGTGTAAAAACGAGAAAAACAAAATGAAAGAGGGGAAGAAGATGAGAGACCTGCAAATCCA
GCGCCACAGAAGCCAGGTGACCAGGGACCCGCGGGCTCGGGTTGCCGTTTCCCGCCCCACCCCGC
GGCCGGCCTGGCTTCACTGGCGCCCTTTGGCCGCGACCACGGGAACCAGCG
CGI: 54 range-chr14: 60173732-60174416
CGGCTAGGCTGCGCCTGCATCAGGCGTGGTGATTGGCAGGCGCCTACGGCCCACGCAGAGATGG
CCGCGTGGGGGTCGGAGCGCCAACTTCTCAGCTCCCCTTCTCCCATGACCCTCACCCCCCACACC
ATCTTCAGCCGCCCACTGGGAGCTTCCTCCACCCCTACCCCCCAGTGCCGGCGAGGCCCCCTGGC
AGATGGAAGCTGCTGACTGATGGGGGCTGCCGAGCGCGAGGGAGCTGAGTGAGGCCCTTGCGCC
CTAGGCGGTTGCACCCCGGACCCCTGCAGCTGTGGACGCCGGAGCTGTGCGCAGTCATCCTGAGC
CAGCCCGCGGGGCCCGATCGGAAGGGACGCCTCTCCTAAGAGCTGGCCTGGAGTCCTGGAGCCT
CCAAGCGGGCAAGTAGGGGCCGCAAAGGCTGGGCTGGGCCTCCGCTGAGGCCCTCGGGGGCCTC
TGGGCCCTCATGGCCTGGAGGCCGCGCGGCCCAAGCCCCTAGAGCAGAGGCCGCGCTAGCCTGC
AGGGGTCGACGCAGGGCCGGAGGCAGCGGCTTTTCCTTCCCACTCCGGGTTGACCCTAAAGACAC
GATTTAACGTGGCGGCGGCAGCTGCCCACGCTGTCTGGAGGAGCAGGGCGCCTGGGATGCGGCG
GCGAAACTGGCCCGGAGGGGGGCCCAGGCCTCACGCGCCCG
CGI: 133 range-chr14: 98781593-98783184
CGGAGGCTCCTTCCGTCCCGGCCTCACCTGGATCCCGGGAACCGCCCGCTCTGTCCCGCCAAGGT
GGGCTGAGGAAGACGGCGCCACTCATCAGGGCAATGGCGGGGGCGGAAGAGGGATCCCCGTGC
GCCCCCAGCACCCGGGACGAATGCCACCCCGGCGAAGCGACCCGACCGGCGCGCACGGTTTCGG
GGAGGGGACCACTTCCACATTCCCCATGCCCGAGAAGGTCACCCACCCGGGAGAGCGCCCAAGG
GCCAGAGAGGAGGCGACTGGTGGAGGAGGGGCGGCCCCGTGCGCACAAAGGGGCCCGGGGCCG
GCAGGCTCCCCGCCCTTGACCTTGGGTCTCCGCCTCCATCCATCTCCGCCGGCTGCCAGGGAAGG
GGGCTGGCCCGGGGCCAGAGGCGTGCTCGCTGGGCTGCTGCTGGGCTGGGGACCGACGGGGGCG
GGGGGGAAAGGAGCCGGGAAAAAAGACTCCAGGGCTGCAGAGTGCTCCGAGGAAGCCTGCATT
CCACCACCTTCAGGAGCCGGTCCCGAGGCGGCTCCGATTCACTGCATGCCCCCCGCCCCCCAAAG
CAAAAGTTGCACGGATGCACGAGGGAAACCCCTTCCCAAAGCTAGCCCTTCGACTCGCCCACCCC
CCGCCTTCCTCGGACCCAGGCCGAGTGGGGGAGGGGAGTGTCAGGGGAGGAAGAAAGGAGCGC
GCCTTCCCAGCGAGGCCAGACGCGGGCTGGGTCAGCTGCGCCTCCGCCCGCCCCTCGCCGCCCGG
CTCAGGCAGCTGCTCCATTAAGCCCCCGAATTATGCATGGACCTTGAGCCCTCTCGGCCCTCCCGC
CCCACCAAGGCACAATCAACGTGCGCAGCGGGCGCGGGTCAAAGAGAGAGGGAAGGAGGTGGA
CGATGGGCGCAAGGCCGTGGGCGAATCAGAGGCCGGCGGCCAGGCGGGGGCCAGGCGCGCCCG
CAGGCCTGGGAACAGTGCCGAGGAGCCTCCCGACCCGGCCAGGGCCAGCCGGGAAAGGGGCACC
GCTCGCAGGGGAGGCCCCCGCGCAGTTTTGAAGTTGCCAAAGTGTCCGCGCCGTGCCGGGTGCGG
GGTCCGGGAGATGCAGCCGCCCCCGCGGAGCTCTGCGCCCCTGGCCTCGGGGAAGGCCATTGTGT
TCTCCTGTGTGGCAATGAAGGGGGCTATGGACCCCAACCATTGAGCCCTTGGAGGGAGGGGCCTG
AAGACCCCTCGGGCAGGGCGGGGGAGGCTGTCTTGCGGGCGGCGGAGAGGATGGGTATGATTTG
CTCGGGATAGGCACCGTCGCGGGGAGGGGAGGAGGCCAAGCTTGAGGAGGGGCTGCTGGAGAT
GGAAGGAAGTGAGAGGAGACTTGCAAGTTGCAAACGTCGGGGAAACCGGAAAATTGCAGTAGC
GGGGAGGCAGGTGGCGCCCAGAGGTTCGAAAGCAGAATTGGGAGGACTTCGATGTCCACCTCGC
AGACAGTGACCCAGGCCCCAGAGGGCTCCGCAGCCTGGAGCCTCGCGTCCCGCCTCCCCGCAAC
ACGCTGTTTTCAGCGTTCCAGACCACAGCGCTGCAGTCTACGCTGCCCTGACG
CGI: 248 range-chr15: 50867751-50870991
CGCCAAAAGAATGAGCGCGGGAGGGGGTCACAGCAGCCAGTTCGGCCAAACATCGACGCTGCTC
CGGCACGCTCCAGCCCAGCGCCTGGGATGTGTCAGAGGTACCTGCGAGGAACATTTGCGTGCGAT
TCCACGCACGCGTTGCATCCCTCTTCCCAAACCCGGATCGCTGAACTGAGAGGTGGCGCAGAGTG
TCTCACCAGGTGCGGGGATTTCTCTCCGCCTCAGGCCGTACGAGCTTCCCTCGCTGTCCCTGAGCG
CAAATGAGCCTCTTCAGTCGCCGAGGCCCGGCCGCTTAGCTCTCGGAGCCTTCCACAGCCCAACA
CCCTGAGCCCTGGAGTAGGCAATTTGCTCCCACAGCCCTGTGCTGGCCCTTCCAGGCACAGGGAG
TCCCCCTTCTAGGGGTAGGGGGGGGCGGGATGAAGCGCACCCAGCCCTCTCTCCTACCCTTCCTC
CTTTGGTCCCTTCGGCTTTCGTGTACCTTATCTCCCGCGCGCCCAGCTCCTTGGCCGGCTCACCTGC
TAAGCGGAGCGCGGACATGCGCTGGAACTCCGGCTCCTGCAGCCACTTCCACATCCTCCGGAAGG
TCTCCCGGCCGGATTTGAGTTTGCTCCAGGGTTTGGGGTTGCGCAGCAGGTCCGAGAGGGTCCCC
TGGGAGCGGCAGAGCACCCTCTGCGCGAAGATGGCCTGTGGGATGCTGTAGCGCTTGAGCTCGGT
GGTGATACGCTGCGCCACCTCTTTGGTATTGATCTCTTCCATCTGCCCTGAATTACTTCCATTGCTG
ACCTGCGCGCCGGTCACCGAAGGGTTGGGCTCCCGGGCTGTGCCCAGGAGTTGCCCGTGGCCCTG
GGCGTTCAGGTGGGCGTGGGGATGGTGCGGAGGAAGGCCGTTGATGGGCACCATGCCGGCCGAG
GTGGGCGTGAGGTGCTGCTCCCCGTGGCGGCCGAGCATGGCCGGGTGGTGGGCTTCGAAGCCGTT
GGGGGTGAGCATCTTGTCGGTGGGCATGGCGGCCCCCGGGTGGGCATAGTGGGGGAGCCCTTGC
TGGGAGTTGTGGATGCTGCCCAGACCGGAGCTGGAGAGGGGCGAGAGGCTCTGGCCCATGCCGG
CCACGTCCTTGTGGTAGGGGGTATAGAGGTTATTCATGGAGGCCAGCCCGCGCTCATCCCGCATG
AGCGTGAAGCTACCGCTCACGTTGCCCGCCAGGCGCTGGTGGTGGTGCGGGTGGTGGTGGTGATG
GTGGTGGTGGTGATGGTGGGGGAACTTGTCCGAGACTGTGGAGATGGGAGGCAGCGGCTGCAGA
GGGGTCAAGGTGGTGTAGGTGGTGGGCATGCTCATACCTGGGGGAGTCTCGCAGGCCATGGTCAT
GGTGGGATGCAGGGGGCCGGCCAGGCTGTGCTCAGGGGCCCGGTGGTGGTGGTGGTAATCTCCG
CCGCCGCTGCCGCCGTCCAGCAGGGACGCCATGCCCATGGAGCGCGGGTGCGCGGGGGGCAGGT
GGCTGCCGCGGTGCGCCACGGAGCTGCGCGCGTGGGGGCTGCCGCCCAGCAGGTCGGCAGGGGC
GGGCACCGGCTCATGGCTCACCCCGTGCAGCTCGCCGATCGCTTCCATGGTCAGCTGCGCGTTCA
TCGTGATCCGGGCGAGCAGGCGGCGGACACAACATCGATGTGGCCAGGCAGAGGCGGCGAGGG
GCGCACGGAGTCCGGTCTTCACATCGGCTGCTGGCGACTGTTGCCTTCCTTCCTCTCACTGTGGGG
CTCTGTCTCTCTCTCTCTCTCTCTCCGTGTGTGTGTGTCCGTGTGTGCGTGTGCGTGTGTGTGTGTG
TGTGTGTCTCGCCTTCCCTCTTACCCCCCACCTTCCCCTCTGCGTCCTCGGCTTTTTTTTTTTTAATA
TTAATTTCCAAAGAGGATCCGCGCCGTTGGGAGAGCGCAGTGCCCCAGCCCGCCCGCCTCGGCCA
CCTCTCGCCCCTCTCTTTCTTAAAATTCTGAGGTCTCCGGCTCCCCTGCCGCGGCCCGCGCGCCTG
CCGCGTCTGCTGCCTGCCCGCCCCGCTGGCCAGCTTGAGCCATGGCTCTGTTACTGTTACAGACTC
TGTGGCCGCGGTTCGGTAGCCGCCGCCGCCGCCGCCGCAGCGGCCCGCCCTCACGCCCGCCAGGC
GCAGCGCGCATGCGCGAGGCCCGCCCCCGCCCCCTAGGTCGCGGCGCGCCAGGCCCTTGGCGTCC
CGGTACAAATGAAGGAGGGGGCCCAGCGCCTTCCCTGCGGCGCTGGATGGCCAGGGAGCTGCGG
GCACGTGCGAAAGATTGGCGCAGAGCGCGACCTGGGGCCGCCGCTGCAATCCCAGGAGACTCGC
GCCTGGCTCGCTCGCCTCCCTTGCTTGAGTGGGCTCTGTCCTCCCAGCCCGGGGACGCTCGTGTCG
GGCTTCTAGCGGCTGGAGTGCTGTGCTTGGAGACATCGCCCCTCTCTCTCCAGTTGCTGCTTCCCG
GTGCAGCTTGCCCGGGGAGCTGGGGACTGTGTCATCACCCCTTCGGCTCTAGCCCACTAAGCTTT
ATTTCCCGGGGGGCTGCTGGGAGTGCGCTTTCCCACCCTTGAATTCACGGCCTATTGGGGGATGG
GGGTTGGGGTGGGGTGGGGTGCAGATTGCTTAAAGGGCTGGGTCCGTTTGGCGGCCGTTGACCCG
GCACCCTTCGTCCTCCCAGATACATACTTGCCCACCCTTTCTCATCCATGCCCTGGGGAGAGGAGT
AATCAGTGCCAAGAATCCCAGTTCGGGCCATACCTCACTGTCCCCCGCCGCCTGGCTCCTTCTCCC
GCTCAGCTCATAATTAAGGCTGTGCGTCCGCTTCGCCGATGACTCGGCCTGTGGAGGGGGGCGAG
GGAGAGCGGAGGGAGTGCCCTGGAGGGTACCCATGTTAACTTCCGGGTGCTGGTGGGGCCGTGG
AGGCTCGGGCCGTCCCTGCGGTTACTCCCAAGGCCCTCCTGCTAAAGCACCCGGAGGCGGTTGCT
TTCCAGAAGTACTGACGCAGACAGGGTGGACGCCGGCGCGCGGGTCTCCGCTTGGCCCCTAGGG
ACGCCCTTTTCCCGGCGTCCCCGAGAGACGCCTCCAGATTTGAAAATCAATTCAGCTTCGGGAGT
AATTTCGCCCTTCCCACAGTCACGCTCCAATCTGGAATCGAACCTGGTCTTTGGGCCTGGTGGGG
ACGTGTGCGGAGGCCCCCAGTTTGAGACGTACACCCGGCCGCCACATGCCGCG
CGI: 226 range = chr15: 58083428-58085812
CGCGCGGCTGCTCCATCTGTGAACGTGAGTGGTCGCCTGGCTCCCTCTTCCGCGGCGGCCGCTTCC
TCATACCTTCACACGGCGCACACCGGCCGGCGCGTCCAGCTGCCTGCCCAGTGGCCGAGGCTCTT
CCCCCTCGCCCAGTCTTGAGCTGGAAGTGATTCCTATTGGCCAAGGTGTCCATGTAAATAGGTGT
GAAAGAAACCAGAGCTGGCCGGGCTCTCCCTTCTCGCTCAGTCCCCTCCCTCTGCAGCCCCCGCT
CCCCCTCCTCTTCCTCCTCCTCCCAAGGCGATTGTCATATGATAGCTAAGAAGTGGCACATTAATG
AAGCGCCGCTACAGGGGTCTTTTCTGCTCCTGTCACCGCTTAAAACTATCAGATGGTTCGAGGGA
GGACATGGAGGCAGCCACCTAGCTCAGCGGAGACGCGGAGCCCACAGCAGCGCCCTCCGGAGCC
CTAACACGTCGCTGCCACCATCCGCGCCGGGACTCCGCAGCCGAGCTCGGCCGCCCGCAGGACGC
TCCAGGAGCGTCGCGGACCGGGCGGCACGGGACGCTGCGGGGCTGAGCTCAAGAGCCCAGGTTC
GCGCCGAGTCCAACCGGACCCGGACGCTGCGCGCGGAGTGCGCGTCGAGTGCGCGCCGAGAGAG
AAGCGGCGCGCAGCGGCGTCCTCCCGGATGCGGACGCGCAACTTGAAGCAACTTTAAGGTGAGC
AGCTCTCTGTTCCGTCCCTGCCCCCTATTCTGGCCCCAGTACCGACTTACTTCCCGGCTATCCTCGC
GCCGTTCGCCGGCTTCCCCTCCCGCGCCCACTAAGCCCGCAAAGTTGCTGGCGAAAGAGTCCGGG
CGCTGGCTGATCGAGCGCCGCAGGCCCCACCCCCGACCCCCGAAGTCTGTTACTCGGTCTGGCTG
ACCCCGCCGGTGTCTCTGTGCATCCATGCTACCTTTCCCTATTACCCACCCCCTTCCCAGATCCGA
GCAGTCCGCCGGCCCGCGCGGACCCAGAGCAAGAAGAGGGCGAGGAAGAAGATGCCTCGGCCC
GGCCGCAACACGTACAGCGACCAGAAGCCGCCCTACTCGTACATCTCGCTGACCGCTATGGCCAT
CCAGAGCTCTCCCGAGAAGATGCTGCCGCTGAGCGAGATCTACAAGTTCATCATGGACCGCTTCC
CCTACTACAGGGAGAACACGCAGCGCTGGCAGAACAGTCTGCGCCACAACCTCTCCTTCAACGAC
TGCTTCATCAAGATCCCGCGGCGGCCGGACCAGCCAGGCAAGGGCAGCTTCTGGGCGCTGCACCC
AAGCTGCGGGGACATGTTCGAGAACGGCAGCTTCCTGCGGCGCCGCAAGCGCTTCAAGGTGCTTA
AGTCCGACCACCTGGCGCCCAGCAAGCCAGCCGACGCGGCGCAGTACCTGCAGCAGCAGGCCAA
GCTGCGGCTCAGCGCGCTGGCGGCCTCGGGCACGCACCTGCCACAGATGCCCGCCGCCGCCTACA
ACTTGGGCGGCGTGGCGCAGCCCTCGGGCTTCAAGCACCCCTTCGCCATCGAGAACATCATCGCG
CGGGAATACAAGATGCCTGGGGGGCTGGCCTTCTCCGCCATGCAGCCGGTGCCCGCTGCCTACCC
GCTCCCCAACCAGTTGACTACCATGGGCAGCTCGCTGGGCACCGGCTGGCCACACGTGTATGGCT
CCGCCGGCATGATCGACTCGGCCACCCCCATCTCCATGGCGAGTGGCGACTACAGCGCCTACGGC
GTGCCGTTGAAGCCGCTGTGCCACGCGGCGGGCCAAACGCTGCCCGCCATCCCCGTGCCCATTAA
GCCCACGCCGGCCGCCGTGCCCGCGCTGCCTGCGCTGCCAGCGCCCATCCCCACCTTGCTCTCGA
ACTCGCCGCCCTCGCTCAGCCCCACGTCCTCGCAAACAGCCACCAGCCAAAGCAGCCCCGCCACC
CCCAGCGAAACGCTCACCAGCCCGGCCTCCGCCTTGCACTCGGTGGCGGTGCACTGACCCGCAGG
AGCCCACGCCCCCTCTCGTTCTCCTCCCCACCACCTCACTCGCCTTCCCTGGCTCCCAGTCCTGCC
GGCCCCCACCTGGGACCGCCACCCTAACTTGTTCATTTCACCTTCGGCCAACCCGCCTTGCCCCAA
GAGAACTTTGTTTTGGACCCAGGAGACCAAACACAAACTTGCAGATGGGCCGAGAGGCGCGTGG
GAGTTGTCCTCGCCCCCACATCAAGGAAGGCCGGGGCCACCTGAGCCGAACCATCCCCTCCCTGA
GGCCCCGAAACCCCCTCCTATTTGACCGGCGGGGGAAACCCTGTCACCCCCTCTTCGCCGAGAAA
AGCGCGTCTTCCCTCCGCCCAGATCGGCGGAGCCCCGAACTCTGCG
CGI: 22 range-chr15: 62984853-62985116
CGCGCCCCTCTGGCCTCCATCACTGTCGTGCCGGTCGTGGGGACAGATGGGAGGGAATTACGGTA
TTTACAGCTGCTCTTCGCCGGCTCTTGCTCCCCCGCGTGTCGACAACCGAAACTGCAGCGAGGCC
CAGAGGCCTCTGCCCACTCCCCTCGGAGTTCCAGGAGGACGCTAAGCGCGAGAAGCCAGGCTCA
GGGAAACTGAACGCCCATACGCTCCTAGTCCCTCTCACCTGGATCCTCTGCGTCAGGTTACGTGCT
TGCG
CGI: 541 range = chr15: 65902540-65909629
CGGGACGCAGGTGCAGGGTGCTGGACGCAGGTGTGCGGCCGCTTACCTGGGGCGTGCAGGCCTT
GGGCTGCGGGGAGGCGGCGGGCGCCGCGCGTGGCCCGCGGGCGGGCGGACCCGGGGAGGGCGC
TAGCGACTGCGCGGGCGGTGGGTGTGGCGCCCCGGCTCTCAGCTGTCAGGCGCGCCTGCCCCGCC
ACTCGGGCTGTGACCGCGCCGTCCCCGCCGCCGCGCCATGCGCTCCAGGCTTCGGCCTCTTCTTCC
GGCCAGTCCCCGGCCTCGGGGTCAACGGCTCAGGGTTCGCATCTCCGCGCCCGGGGTCCCGGCGC
CCCGTGCCCGCTGAGAGCGGGAGGGGCCGCGGGTGGGAGCGCGGAGAACCAGGGAGCACGGCT
GTGCGCGCGCGCGGGGAGGGGCCGCGCCGTCACCCGGAGAGCCCGGCGGATCCCGGACTCCCGC
CCGCCGCTCCTCCCGCCGCGCACCTCCGCTCACCGCTCGCCCGGCTCCAGCCGCCGCCGCACTGT
GAGTACCCACGTCCGGTAGCCCGCCGCCCCCGCGCTCTGCTCCTGCAGCTTTTTTTTCGTTTAGTT
CTGATTTGGGCTCCTGTGGCTGTTGTTTTTATGATTATACCTAAAGACACCACCGCCTCGCTCCCA
CCACTGCCAGTCTCCCGCTTCGCTGCCCTCCCCAAGCTCGTCCATCTAGCTCCGGGAGCCGACGG
GGGTTCCTCAAGCTGCTCTGGCTGCTGCTCCTTGTCGCTGCATATGGCTCTTCGCCTGTGCTTTCCG
CTTTGGCCCCCGAAATTCCGTTCCTTTCCTAGGGACCCCCTTCTTCTGACTCCGGGCCCGGACCCC
CCTCCTCTCACTCAGGGAAAACCGCGCAGGGACCCAGGACCCTCAACTGGGGTACGCTCTCGGCT
CGGGCGCTGTCTTCACTGTATGCGGGCCAGGCATAGTTGCCGATTTCGAGAAACCGAGAAGCTGT
CATTTCCCAGCGCTGCGCGCCCTCCAGCCACCTCCAGGAGGGCGCGAGGCTTGTCAGCCGGGGAG
TTAGGACAGTGTTTTTGCCTCCTACAATGATATAAAGGACTTCAACGCGAAATAAGGGTCAACTT
ATTTTAGTTCGATCCTCGGAGATAAAGATGAAACAGACAGATAATACATAAATAGCTTGATACTT
GAAGAAGGTACAAAACAGATGAGAGAAAGAGGAACGAGCCGGCAGAGACAGCTCAGGGACAGA
TAAGATCGACAAATGAGGGTCTCCTAGACAAATAGAGAAGAAGGGAGAGAGACAGATAAACAG
AAGATTCCAGTCTCCTTGCTGAAGGCTTTTGAACTGGGATCCCCTGTCTGGAGCTGCCCCTCTGAA
AACGGAGTTACATTCAGGGCCGACTGCAGGGGAGGAGCCCTCAGGGTTGGGCTGCCCTGGCCGC
AGGAGCGCCTGCAGCCTTGGGCCGGATGGGAGCCTGGCTTGTCTGAGGACATGTGGCTGGGTCA
GGCGACCTAGAGGGCCCCAAACATCAGTCCCCAAGTACCCCCGCTGCTGAGGTCTGCCTGCACAG
CTCCCTCCCTGCCTCAGCCAGAAAGCAAAGTGCAAGGACCAGCAGCAACTTGTGTGCCTGGAGCC
TTGCAATGGCTACGAAGATGGCTGAGATTCCTTCTAGCCCATATGAGCCGGGACAGCGAGGCATG
AAGGATACTCAGAGAGGTGATGCCCCTCAAAGAGGAAGCCCAGAGCCCCGAATCCTGCAGTTGG
CCCGAGTGGGAAACGTATATCTGAAGGAGGAAAGCTCAAGGAGTGGGCGACTGGAAGCTAAGA
AGTGGATCCCAACAAAGTTCCTTGAGAACCTCTCTGTCCCACACGTTGACCTCGGTGGAAACCAG
CTTTGGTCTGGAATACAGCTAAAAATGCAGAAAATGTGGTTAGAACTGCAGGATCCTGGTCTCAC
TAATAGCTGGAAAGAAAACGGTTTATATATCCAGCCGAAATCTTTCTGCTCAACGAACGCGGGCG
CCAGCCGTGAGAGGAGAAGCTCGGGTTTGTGGAAACTGGCAATGTTGTCCCCTGAAGCCGGCTCG
GGCACCAAGCTGGGTGCGGTGGGGGCCGCGCTAGGTCCGGGTGCGCACCGAGCGCTTTGGAGAG
GGTCAGGTTCAGCACCGCGAACAGCGGTAGCCTCCGCCTCTCTCCTTCCCTGGCAGACACGGTTC
CCTCCTCCTTCTCCTCACCCCCCTCCTTCTCTTCACCCCCCTCTACCCCCTGCTGTCTGGAGATGGG
CAGTGACCGCCCCTCCCCCCTCCCCACCCCTCCTTGCACTGCCCCCGCCCGCCCGCGTGCATGCGC
GACTGAGCAGAGGGGCGGCCGATCCCCGAAGTCCGGGCTTCAGGACCCGGGCCGGCAGCACCGG
CTGCGAGGGTTGCCGAAGGCGCACGGATCTGGGCGCTGAAAAAGCCAGGATTTGGCAATGGCTT
TGCTGTGTGGCCTTGGGCAAGTCACTCTGCGTATCTGGGTTTCACTTCCTTCCCAATCCGAAAACG
GGATTGGGTTCCTTGCAGCCCGGGCATTCCTGAGGTCTCCTTTACCCCTTCTCCTCCCCCAAGCCC
CGGGGGCTGTTGTTAACGGCGCCAACATGGGTCTGAGTAACAAAAGACGCCTGTCCAGGGGGTG
AATGAGTTTGGACTCCCCACTGCCAAGTATCCCCCGCGCGCCCAACTCGGAGCGCCCTGCTGGGC
GGGCCCGAGCCTCGGCGGCGGCGCTGAAAATGGCTCATCTGCTCTCTGCTTTCTCTATTTCGCAGG
AGCGGCGGCATGGAGGCTCTCACCACTCAGCTGGGGCCGGGGCGCGAGGGCAGTTCCTCGCCCA
ACTCCAAGCAGGAGCTGCAGCCGTACTCGGGCTCCAGCGCTCTCAAACCCAACCAGGTGGGCGA
GACGTCGCTGTACGGGGTGCCCATTGTGTCGCTGGTCATCGACGGCCAGGAGCGCCTATGCCTGG
CGCAGATCTCCAACACCCTCCTCAAGAACTACAGCTATAATGAGATCCACAACCGCCGCGTGGCC
CTGGGCATCACGTGCGTGCAGTGCACGCCGGTACAGCTGGAGATTCTGCGTCGGGCCGGGGCCAT
GCCCATCTCGTCGCGCCGCTGCGGCATGATCACTAAGCGAGAGGCCGAACGCCTGTGCAAGTCGT
TCCTGGGCGAGCACAAACCACCCAAGCTGCCCGAGAACTTCGCCTTCGATGTGGTGCACGAGTGC
GCGTGGGGCTCGCGTGGTAGCTTCATCCCTGCGCGTTACAACAGCTCTCGTGCCAAGTGCATCAA
GTGCGGCTACTGCAGCATGTACTTCTCGCCCAACAAGTTCATCTTCCACTCGCACCGAACACCCG
ACGCCAAGTACACGCAGCCCGATGCCGCCAACTTCAACTCCTGGCGTCGTCACCTCAAACTCAGT
GACAAGTCGGCCACAGACGAACTGAGCCATGCTTGGGAGGACGTCAAGGCCATGTTTAATGGCG
GCACGCGCAAGCGGACCTTCTCCCTACAAGGAGGCGGCGGAGGCGGTGCCAATGGCGGGTCGGG
TGGGCAGGGGAAGGGTGGTGCTGGCGGCGGTGGCGGCGGTGGCCCAGGGTGCGGTGCAGAGATG
GCCCCAGGCCCGCCGCCCCACAAAAGCCTGCGCTGTGGCGAAGATGAGGCTGCCGGGCCTCCGG
GGCCACCTCCACCCCACCCGCAGCGCGGACTTGGCCTGGCGACTGGAGCTAGTGGCCCGGCGGG
CCCAGGAGGGCCCGGTGGCGGCGCCGGCGTACGAAGCTACCCGGTGATCCCGGTGCCCAGCAAA
GGCTTTGGGCTCCTGCAAAAGCTGCCCCCACCACTTTTCCCCCATCCTTACGGCTTCCCTACGGCC
TTCGGCCTATGCCCCAAAAAGGACGACCCGGTTTTAGGCGCGGGCGAGCCAAAGGGCGGTCCTG
GCACTGGGAGCGGCGGCGGCGGCGCGGGGACAGGCGGGGGTGCGGGGGGCCCGGGAGCCAGCC
ACTTGCCCCCGGGGGCAGGGGCGGGCCCGGGCGGCGGCGCCATGTTCTGGGGGCATCAACCCTC
CGGGGCAGCCAAGGACGCAGCGGCAGTGGCTGCAGCGGCCGCCGCCGCCACTGTGTACCCGACG
TTTCCCATGTTCTGGCCAGCAGCAGGCAGCCTCCCGGTACCGTCCTACCCCGCTGCTCAGAGCCA
AGCCAAGGCCGTGGCGGCAGCCGTGGCGGCGGCAGCGGCGGCGGCAGCGGCAGCTGCTGGCAGC
GGTGCCCCAGAGCCCCTGGACGGTGCCGAGCCAGCCAAAGAGAGTGGCCTCGGCGCGGAGGAGC
GCTGCCCGAGCGCTCTGTCCCGCGGGCCCCTGGACGAAGACGGCACGGACGAGGCGCTGCCACC
GCCCCTGGCCCCGTTGCCCCCGCCGCCCCCGCCGCCCGCACGCAAAGGCTCCTACGTGTCGGCCT
TCCGGCCGGTGGTCAAGGACACCGAGAGCATCGCTAAGCTCTACGGGAGCGCCCGGGAGGCGTA
CGGCGCGGGGCCTGCTCGGGGGCCGGGACCCGGCGCTGGGAGCGGCGGCTACGTGAGCCCGGAC
TTTCTGAGCGAGGGCAGCTCCAGCTACAATTCCGCCTCGCCCGACGTGGACACCGCGGACGAGCC
CGAGGTGGACGTGGAATCCAACCGCTTCCCCGACGACGAGGACGCCCAAGAGGAGACCGAGCCC
AGCGCACCCAGCGCAGGGGGCGGCCCAGACGGTGAACAGCCCACTGGACCCCCTTCCGCCACCT
CCTCTGGCGCGGACGGTCCCGCAAACTCTCCCGACGGCGGCAGCCCCCGCCCCCGGCGCCGCCTC
GGGCCACCCCCAGCTGGCCGGCCCGCATTTGGGGACTTGGCAGCCGAAGACTTGGTGCGGAGAC
CTGAGAGGAGCCCGCCAAGCGGCGGCGGCGGCTACGAGCTGCGAGAGCCTTGCGGGCCCCTAGG
AGGCCCCGCGCCGGCCAAGGTGAGCCCCGCGCCCGCCCCGCCAGTGGCGCCTTCCCACCTACCCT
GTGCGCGGCAGGGCTGCGGGGCCGGGCGGAGAACGGGAATTTTGTTCCGCGCAGGCGTGGGAGC
AGCAGGCCTCGGCCACTCTCCGCAGGCCCAGCCTTGGGCGCGCTGCGAAACTGGGCCCACCCGCA
CCCTCCAGTGAGGGTTTCGAAGGGAAGGGAAGCTGGGGGCGGCGGGAGGACTGGGGAACTTGTG
TGCTTTGTTTTTGTTGATGGGAATGGGGAGGAGACCGGCTGAGACTGCCAGACACAGAGACAGA
AAAGGGGTGTGGAGAAAGAAAGAGGAGGTAAGAAAGGAACCCAGAGCCAGAGAAAAGCGCTCG
CCCGGGTGGGCATCAGAGCCCCAAGGAAGAGGAGAAGAGAGGACAGTGTGGCCTCTCCCTGAGT
CGGGCCCCAGCAGTACCGGGTCCCCGGAATCTCCCACCACCCCAACTCGCAGTTTTCCGAGTCTC
TTCGCACTCCCGCCTTGGGAACCACAGGGTTCGTTGCTGGGAAGCTGTCTCGGGACCGCGTGCGG
AGCGGGAGGGGGGCTCTCAAGCTGGCAGGGCCGGGGCTTGAGGGGCAGGATGCAGCCAGGCTG
GAGTCGTTGGCCATTTTGAAAGTGAGTGCGCATACCTGTACATTCGGTTCATTAAAACACGAATC
ATTAACCGGATGCGAAAGGCGTCATTTATGCCGCGGACGGGGCACTGGCGGGAGGGAAAATGCC
TAAAAGGAGGCGGGTGCAGAGGGTGGAGGACACTTTGAACGCCCCTCGGCTTGGGAGCCGCTTT
GTGTTTAGAGTAATTTTCCCGAGGGCGCTAACAATTACCCTGAATCTACAAGGGGGCAAGGGTGC
GCCGCGTGCGTCACTGTGCCCGCTCAAGTCCAGCGGGCGCACCAACCTTTGCCACTCCGTCGGCT
TTCCCTCTTGGCCGCGGGGTAGGGCTGGGCGTCTTTGGGCCGCCGCAGGGCGCCACCCTAATCGC
CTGTCATTTCTCGGCCGTCGCAGGTGTTCGCGCCCGAGAGGGATGAGCACGTGAAGAGCGCGGCG
GTGGCGCTGGGGCCCGCGGCCTCCTACGTCTGCACCCCCGAGGCCCACGGTAACGCCTGTCGCGG
CCGCTGGCCACTGTAATGGGGGAACCGCAGACAGAGGGCCGGGGTCAAGGAAGGCCTGCGAAG
GAAGACAAGGAGAGAGACGCAGAGAAAGAGGGAGAGGGAAAGAAAAATAATGAAAACAAGGA
AGCTAAGCTAGTACTCCCAACAACTTCCAATCTTTAGTTACTTAAAAACAACAGTGACAACGCCC
TCTCCACCACACGCACACACATGCTGCAGTACACCCAGACACGGCGTTAAACCCTTAACAAAATC
ACGCCCGACACGGCCTTCAAATCCTCTCATTTATTCAGCAAGTGTGTTTGGCCGCGGGGTCCCCTT
TGGCTAGGCCGGATCGGGGCGGAGGATGGGGGCACATGGCCGCGCGCTTGGGTACCCCAGAAGC
GCCAGGGTAGCTGAGGCCCATCGCGGGGTGGTGGGGGCCGCCCTGCACTTGCGCGCCTTACCAG
GTTCCTCACAGAGGAGGGGTTGGGGGCAGCGGAAAATCGGGCAGGTCGAGGCAGCCGAACCCCG
GACGATGTCCCCCCACCCACCCCGAAGGTCGCAGCCTGGGCCGCGTTCTCAGCAGGAGTCGGGCG
GACAGACCCGGCGGCCACGCGCGCTCGCGGTGCCCCAGTATCTGCGCGCGATGTAGGTCGCTCGT
CCCTGGTGGGCTCGGCTGCTCGCCTAGCTCTTTTCCAAGGGGCTGGGGCGGGGGCCTGAAGCCCG
GAGATGAGCAGACCAGGCGCGGGGGTGGGGGGTGCCCGGCCCAAAGCCTCAGGGCGCGCTCCTG
GGCGCACAGCCGGGAACCAGAGGCGGCCACGACGCTTTTAATTAGGGCTGGGGCGCCCCGACCG
CGGCAGCTCCGGCAGTATCCGGGTCACGGTTTCTTTGCCTCTCTTGAAATTCGGTTTGTTGCCTTC
AAGAGCCAGATAAGGAAGACAATCACTCGCCCGCCGATGATTTGGAAACG
CGI: 216 range = chr15: 72206924-72210097
CGGGAAGTAAAGCCTCGGGACTTTGGTTAAGCGCGCCCCACTGGCGTATCGCGCCGCGGGACTGC
AGAGCCGTGAGCAGCGGTCGCCTTGGTGCAGGGTTCCAGCCACATCTTCCTCGCCCAGCCAGCAC
CCTCTGCCGTTGACCCGCCTCTGCGCCCGGCAAGCGGCTTCCAGCAGGGGGCGCGCGCGGACCAG
GCTTGGGCCCTAGAACAGCGCGGATGGAGTCGCTGGAGCAAGTCCCCAGATCCAACCGGTTTCA
ACCCTCCCCACCCTCCCGACGCTCCGGGTTCGCGACGTTGAAGTTAAGGGTCGATCCGCAGAAAG
CGGCCAGGGGCTCCAGCTCTCCATTCCTGGGTCTGTCTGGGGTCGGCTCCAGCCTGGTTAGAAGC
CTTAGTCTGGATTCGGCAGATTCTGAATCTGGGACCCTCTGCGCTAGCGGCTTGGAACCTTGTCAC
CCTCCCCTCCCCCACCCCTACTTCCACACACCTGATTAGTTGTCTGTTTCTTTAATGATCAAAGAC
GTGGGCGGCGGCGGGATGAGGTCTTGGTTCCCGGCTCCACAGCCCTCCCTAACTGTCATTATTAA
CGTTATAAACATTAGACCCGCTTCTGCGCGCCGGACGGCGCCGGACGAGGTGCGCGCAGTCTTCT
AGCTGAGCTCGGAGGCAGATCCAGAAGTCGCGGCTCCCACCCCAGGCCTCGGCGGACTCTGCCTG
GGGCGACTCGGGCTCCAGCCCTGCCCGGGCGGGCACTGGGCTCTCCAGGGTCGAAGGCAGGGGT
AAGGGGCGTCTTTCCCCCAGGGCAGCCTCCGGGAACAAAAGCATTTGCTGTAGAGTGAGCTAGA
GCCTCCGGGCCCGCGGGAGTCAGCTCCCGCCCAGGGGTGGTCACCGCGTCCTTAACCACCCCAGG
AGCCCCGTCTCCCTGCCGAACTCCTTGGCTTCTGCAACCCTGTCAAGACAGCAAGGAAAGGGGGT
CTTCCCTGGTCCTCGGGCCCCGAAGTTTCGGGGTTGCTTATAGGACGGGTTCCTGCAGTCCAGGG
AAGCTCTGGGCAGATAGCGAGCCCATTCTCCCTTCCATTACCCAGATTCTGCCTCCCTGCGGAAG
GCAAAAAAGAAAGAAAGAAAATAGGTAAAAACCGGCGGAGGGCCTTGAGCCTCCCCGCCTGGC
GCCCCTCACTCAGTCCCGAAAAGTCCCCTGGACACGCCATGCCGGACCGGACTCAGCTCCCGCTG
CTGGGCCCCTGCCTCCAAATATCCTTCCCAGGCACAGGCTCCCAAGACCGCCCCCTCAGGGTTCC
CAACACCCGGACAACTGCCCAAGACGCCGCTCCCCGCCCCCACCCCCACCTTGTTCGGCAGACAA
AGAAGGGTGTGCTGGCCCCGCCGTCTGCCTCCTTCTCCCGACCCCACAAGGCCTAGAAACCTCAG
GGACTCACCCCGGGCTAGGGACCCAATCCTGGCTGTCCCACCACAGGATCCCCGGCAGGGACGG
GTCACAGTGCTCTCACCCCTCGACCATTTTCGAAGACACCTTCCCTGAAAGGCGCCTTGCGCCCTC
CCCATGGGTCGGCGGGGGGGGACTCCAGGCCCGAGCAGGCGGTGTGAAGTTCTGTGTTCTGAACT
GGGGCTGAGCAAGATGCGATGGTCTCAGCCCGCTGGGCCGCCCGTAGCGACGGCAGGAGTAGGG
GAGAGGGAGGGACGCTTGGAGTGTGAGCGCACCAGTCTGTTCATATTTAATTTACAAAGCAGCCT
CGGAACCCCGGGCCGGGTGGTCTCTTTAGACGCTGCGCTCTTAGCCTGTCTCTCTTCCCCACCCCC
TCCCCTAGCTCATTAAGATGCTCAACACTCAAATCGGGGTATTGATCTCCACGGAAGCCCCAAAC
CCTCGCCATCGAGAGACCCCCATGGCCCGGGGTGATGGCTGTGGGGCTTGGTGCTCCCAGAGAGC
TCAGTGGCTACAGAATGGGTGGGGATTCTGCGTGTCTCCCGGAGCCTGAACCCCTTTCCTGGTTAT
GGCCGGTAGCTGTCTCCAGGGCTAACGTGGGCAGCGCAGGGGGGCGGAAACCGGGTTTTAGCCA
AATGCCTCGACATCGCCGCGCCTCCGCCTCCTCGTCGCTGAAAGAAATGTCGGGGTTTCATCAGA
GCTAGGGAGCGACAGTCGGGAACAGCGAGTCTGCCGAAGCCGGCTGTTGTGTGAGGGTGTGAGA
CGGCGGGGCGGTGAGGGGCCACCGCGGCTTGGGGGATAGTGCGTGTGGGGTTGACCGTGTGTCT
GCTTGAGAGGCTGTGAAGATATGGGGGGCAGATATGGGAGAAATGCTCGGGCCTGAAGTCCCCA
GCCCACCGTGCTCAAGAGTAGCGGACGTTTTGCCACCATCCTTGTCTGTGCTACTGTCTGCTGCAG
CTTCCGTGCCCCGTTCTCCTGGAGCAGGCAAGACCTGGAGTGAGGTGCTTGGGTGCGCTCGAGAG
AGCTTCCCCCTGCTCCACCTGTCCCGCGGTGCGCGCAGGCCAACGCGTCGGGCAGTGGGCTTCAA
GCGCTGGTTTAGCCACAAAAGACCAGAAGTAAAGAGTTCCGGCTTAAGAGGCTGGGCAGGGCTG
CGGTGGGCTGGGGAGGGGGGTGTCCCTTCCCAGCACGCCCTGCAGGGCTGTGCGTTCTGGTGTCG
GGTTAGACTAGCAGGCGGGGCGGGGGGGTTGGGGCGGCGGGGCGGGGGAGACTAGGGCTTATAT
CAGCCCAGATCCAGGCAAAAATGGTAGGGAGGGTGCGGCGCTCTGCTAACACTATCAATTATGC
ATCATGTTGAACGTGGCTTCGGGGAGGAGGCGGCTAGCAGCGGGGGGTGCGGGAGGGAAGGGTC
CGCGCGAGCTCGGCTGCGCGCAGCTCAGCGGGTCCCGCTCGAAGTCTGTCGGTGCCACCGCCTGC
ATTTGCAAAAAGAGTTTAAAGGCAAAGACACGCCTTCCCCCCCCACTTCAGCCGCGCGCCTTTCC
TTCCCCCAAATTCCTCAAAGATGGTTTGTCTCACGTGTTGCAGGGCGTAAAAGCGGCTTGCATTCA
ATTAGCAGCGAAGCTCGCGGGCGCTGGCGGGACAGGCGCGTGAGGGTGAGTTCGCGTGAATGTG
TGTATGCGTGTGCGAGAGGAGAACGGTAAGTGTCCCGGGTGCAGGTGTGCCCGTGAAAATGCG
CGI: 32 range = chr15: 74414579-74414893
CGGAGCTCAAAAGCCGGACGCTACTGGACGAGGCTTAAAGCGCTCGCCCCAGCGCGGGGCCGGT
GAGGAACGTTCACCGTGGACCATGGTCGAATAATGTTAGCGTGCACTCGGGGCCTCGCCCTAGCC
TTCTGCAAAGCCTCGGCCTGCGCCCTCGGCTGCATCCGTTTCGACGCCAACCAACCTCATGCTCTT
AGTAGAACGGCAGTAGCGACCGCGCCCTCAGGCCCACAAGCCCGAAGGTCTGGGCAGGGTCTGG
AATGGCGGTGGAGACACTTTATCCGCAGGCGTTTCACCTCGCGCCCCACCCAGGCG
CGI: 109 range = chr15: 88158407-88159577
CGGCATCTTGGCCGGGTTGTAGGAGCGAGGGAGAACGCGACTGTAACGCGGGCGTTGAGCACCT
AGAGTTCCCCTTCCTCTTCACTGAGAGCTGCGGCGGAGGCTTTAGACCGAGGAGCGAGATCTAAG
GTTAAAGAAAGGGCCTCTGAGCCCAGTGTTGGCCAGAGACCCCACAGGCGGGCCGGGTTCGCCC
TTTGCCTTCGGCCGCGCCCCACCCACCAGGGACAGAGTGGCCGCGGCCGAGCCGGGTCCACCCGC
CGCAGCTTAGCTCCGCTCCGCCGCCCTCCCCGTCCGTCCTCCCCAGGCTCCCGTCGCCCTCGCCCA
CCCTGGGGCCCCCTCCCCAGATGGGAGCCCCTCCGACCCCTGCGAGGCCCGCTGGGGACCCACTT
GACCGCGAGATCGTAGTGGGAGCTCGGCCAAAAGAATTCTTCGCTTTCCCTGGCCCTCAGTTTAC
CCATCTGGGAAGAGGGGCGTCGCGGCCGCAGAAGTCCAGCTCCGCACGCGCCAAGCGCTGGGTC
TGCAGCTCCCTCGGTTCCCGCGCACCCAACTGCCCCACGACCCCTACCCGCGCCCGCAGCCCCCG
CCCTCCCGCCCCACTTGCCTGGGCCGCCGGGCGCTGGAACCTGGACCCTGGACCCTGGCGGGTTC
CGAGCTGCGCCGCCGCCGTCCCTGCCCCTCCAGCACTGGACTCCTCCTTTCCCGTCTTTTTAAAGG
GAAGTAACCGGACCCGACCACTACTCCACCCGATTCACCCCTAGCGGGCCGCCCGCCCGCAGCTC
CGCCCGGGCTCCTCCCCCGTCCGCCCGCGGCTTCCCGGGAGCCCCCCAGGGCTCCTCCGCTCCTCC
GACGGCCCGGCCTTCCCCCCGGGGCCTGGGATGCACCAGGGCTCCTGCTGCCCCCGACGGGGTCC
CTGTCGCCGCAGCCCGCGGGACCCGCCGGCCACATCTGGACGGCATCTGCCGCGGGGGAGGGGC
TCAGAGCCTCGCCCTGAGTGTCCCGCAGGGAGCCTTGTGCATCCGGGGCAGGAAGCCAGCGCATC
CCGTGCAGACGGGTGGTGCAGATAACGGACCCGCCACAGCACGCAATTCAACTTTCTTGGTACCT
GGGACTCGTAGTTCAGAAGGCGCAGTAGCACGACCTTGCGAAAAAGCACCGGAGTCGGGAACAC
GCG
CGI: 21 range = chr16: 727072-727328
CGCCACCCGCACGACGGACGTCGGCACCACACGAGCTGCCCTTGGCAAAACGCGTTGTCGGCTGC
TGGGATGGATTTCAGGTTAAAGCAGAGTCACCAGGCCGACCACTGCTTACCTTGTTAGCATCTAG
AACCTTCTTCAGCTCCTCGTGGCTCTGCTGGGTGATAAGCACGGTCTCTGCGGAGGTGATGCAGC
CGCTGCACGCCAGGCAGTCGTTTAGCGAGACCTTGGCCTTCTCCAGCCTCCGGGTCCCGCCG
CGI: 61 range-chr16: 47868915-47869809
CGGGTCCACTCCTCACTCGCGAAGTCCCTATCTATCTCTGTGGGTCCCGGAGGCACAGGGCTGCA
AGGGCGCCCGCTAGAGCTGCTCCACGGCTTACCGGATTGCATGCGGAGAAGAAACGGGCCCGGG
TTTCAACAGAGGCCCCGTGCTCCGTGAATTTCTAACTCGAACCAGCCGCTGGGATAAATCCGAGA
AACGTTGTTTATTGTGCTATTATTAACTTTCCCTTCTCCCCACGCCCCTAGAGGAAATAAAATTCA
TTCCTGAATATTGGGCCAGGAAAACGTTTCTCTGAAAGGAACCACAACTGCAGGGGACCACCCG
GCTGCAGCTCCCAGCTCCCGGCCCTTCGCCCTAGGCTGCCCACTCGCTCCGGCTCCCGACCTCCCC
GCTGCCGAAGCAACTGGGTCAGCCTTTCAGACTTAAAACGAAGTCGTTTATTTCATTTTTGCTTTC
ACATTATGTTCCGATACAATCAAAACTCCTGGACGAACCTCTTGAATAAAGTCCTGTGAATGAGC
GGAATCATAGGTCCTGGGCGCCCGAGTATGCGGCGGGTGGACTCCGGATTCAGAGGCGGTGTAT
AAAACATACGCTGCTTCGGGAACTGCGAGGGGGACGCGGTGGGAGACATCCGGGGACAGAAGG
CCTGAGGAGTCTGCCGCTCCAGGCAGGGTCGTTTGGGTCCTCAAACTGGGACTATTCCTGATGCA
TTTGGAATGCAAAGAAAAAAATCAAACTTCGCTCCTGGGCCGGCGGCCGCGCCTGGCCTAGCAA
GATTTCGGCCACATTTCTTACGTTTCCGCCAAAGCTGTGAACACAGGCGAATGCAGTGCTGGTCC
CCGGCCTGGCGCAGGAAGGAGCGCAAGGCCGGCTTTGCAAGGAAGCCCTCGCG
CGI: 179 range = chr16: 53527803-53530347
CGCGAAGGCTCGGGGCTGCGAGCCGAATGCCCGCCGAGCTGCCTGGGAACGGGAACCGCGCACC
CGGGAACGCCAGGCGTTTCTCTTCATCCAGGAACGGCGCGGAGAGCCTCCGGCTGGGGAGCTAA
ACCGCTGGGGACTCCGCGGCTGCTGCCTGAGTCGCTGTCCGCTGCCCGCATCCCTTCCGCCCTGG
GCCTCTGCACGGTCTGCGGTTTTCTGTGCGCACTTGGTCTTCAGTACTAGCACCCAATTACGTCTG
GGTTTTTCTTCTTTACAGAGCTGGGTTTCGGTGGCCACCAGCTTTTCTGGTGTTTAGTGACTCTGA
GTTTGGAGGTGGCCTACCAGGCAAACGGGGATTCAGGGCATTTAGGAAACGTCTTCCGCGCTTAA
TCCCAGAAGTGGTTGCGTGTCCGTACGATCCCAAGTTCCTCCCGGAGCCTCTTCTTTGCTGCGTCT
CGGGCCCTCCAGCAGGCGAAGCCTCTTAGACGCGCTGGGGAAACTTCCGGCGTGTTCGGGGCTCA
GGGTTTCTTCTCAGGCATGGATTGGGGCGCAGAAGTTGCGCGAGGCAGCGCCTAAGGTCCCGAG
GTGCTGAGACTGTGCTGGCGTTTGCCACCTGCCCCTGGCTAGGCCGTTTCTGGGCCCCAAGAAAC
GCCTACCTTGGCACTTAGGGACCAGAAGCCTCTGGATGTCTAGCAACAGGGGTCACGGGATCACT
GCGTGGGGTCTCTGTAAGCAGTCCCCTGAGGCAGTGCAAAACCGGAAACCTGCTCTGTGCGGGGC
CGAATCAGTTCATGGGATTTGGGAGTCAGGAGAGACGTCTTTCTCCTCCCAGCTCCTACACTCCG
GGTACCAAGGCCCGAAATGCCGTTCCCCCAGCCCGGGTGCGGGGTCTGCAGCAAGGCCTCCTGAT
TTGCAAACCCCTCCGGGCTGCTTATTCCTGGTGCCATGCGCAGCCCTTGGGACCTAGAAGATTGT
GGGGGAGGGAAGGTCGCACGTGCGGTTTTGGCACCGACCGTGCCTCCTAGTCCACCTGTCCCCCA
CAGCTAGCTGCCAACTCGGACGGAAGCGCGGAACAGCGCAATGCAAACCGCCCAAAGTGAAAGG
AATGTAATTGCGCTCCCATGACAGAGCCAGACGCGGATGCAGTTTAGGAAGGGCGCCGCCTACC
GGCCCCTGGGAGCCATGCGATTCGAAGGGAGGGGGACCTAGAGGAGACCCCCGCTATCCCCCCA
CCCACCGTGGGGCCTTAGCTTTGAACTCCGGCCCGGACAAACTTAAACTGCTTCGCCACCCCAAC
GCGCCACAGCCCTGGACCTAAGACCCAGTTAGCATTGGGAATTTGGGGAGCAGGGCCCACGACT
AGAACAAAGTTAACCCTACCGGTTCCCGCCACGGCTTCGGCACATTTCAAAAAACCAGGCGCAG
GCAATTGAGAAATACGCTGCCGGCTGAAACAGCCTGCGGGTGGGGGCTGCAGCCGTGCGCGCCC
GGCAGTTCCCGTCCCGCATCAGGTGTACGCACTTCCACTCCTGCGGGCCCTTCCACGCTCCAACTC
TGGACCCCGCGCAGTTTTAATCTGCGGTTTGGGAAATGGGGGTGCTACCGTGCAACCGCGCCCTG
AAAGACCGTTTTGGTCTTAAGAGCTTTTGGCCTGTGGGGTGGACATCTGTAGAAAAAGGAAAAAC
AAAACAAAAGTAACCTCCCATTGCGTCGAACCCTCCTATTCCGAAAAGAACTTTAATGAGGTTGG
CTTGGCAAGGCCTGCGGTGCTTACCTCGGCTTCGCCCTACCCCGCCAGAAGCCCTCATAGGTTGT
ATCCACTGGTCTCCCCCAGGTGCAGGGTTATTAGGGGAAAGGGGGGCGCGCCCTCGGGCTGGATC
TTTGTTTCCCTCGTCGCCCGGTCATCAAACAGGAGGGAAATCGGGCCCGACTGGGACCTTGCTGC
CCGCCTTCCCCTTAAACTGGCTAAAGCTTCAGGACTGTCCCTAGACCCACCCCGCGGGTCTTCCTT
TGTGTCCAGGGCATAGCCGATCTCCTTTTCGTTTTCACGAGAACTGCGACTTGGGCCTCGGGCACT
AGGCGAGCCCAGGTTGTGGCCTAACAGCAGATCGCCTCGGGAGCTTGGCTGCAGCTCTGCCCGCA
CCTCCAGCGCTGGGCGGCCTCTCGGGGCCAGTGGGGATCTCTGGCCTCGTGTAGGCCACGGGCCC
CAGCCCTGGGTCCCCAAGGCCGCTCGCCGGGCAGCGCTCGTTTCCGGCACCGGGACGAGCCCAGC
GCGCTCAGACACCACTTTCCCGGTGAAATCTGCTTTTATTTGCTCCGAGCAAACCTCGGGCTCTCA
GCGCTCCCGCCTGATGGATGCAAATGTAAATGTGCACTTATTTAATTGGATCAGGCCCCAAGATA
AAAGAGATAAACGGCTCCCCGCTTGCTAACTTATTTTCCTAGGCATGCAGCGCCGCGTGGAGGGG
AAGCTAATGAAGGAGCAGCGCGGTGCTGGGCACTCGAGCTCTCCGCAGCCGTGGCACGCTGGCC
GGCCGGCCCCGCG
CGI: 78 range = chr16: 66071900-66072846
CGCAGGCCAACGCGAAGACACGATGATCCCAAAGACCTTAGTGTCTTGGCGGGGGGAGGGGGAA
GAGGTGGGGGACTTTTCCGCTTAGCTTCCCTAAGGGTTGCTACGCGACAGCTCTCGGGCTACGTC
AGGGTAAAGAGGTTTGTATGGCCACACCTCGCACCGACCAACTACCCCCGAAACACCCCCTCCTC
CCAAACTGCAGGGTGAGCACAGCGAGAGAAAAGGTGGCCCAAGCAGAGGCGAACAACTGCGGT
TCTTAAAGCCTTGTTTGAGTTTGGGCCCGCGCCGAGCCACAGCTAGAGGTAGATCCCGCTCCTGC
GCGTCCGCTATCAGCCCCAGGATTCCCAGAGAGGCCTCTCAGGCCCTTCAAGACCCCCCCTTCCG
GCTTCCCGGCCTACACCTCTGAACCCTCAGGCCCCGGACAGCCTCTATCCCCGCCTTTCGCGGCCC
CGGCTCACCCTCTAGCGTCTCGCACTGCACCAGGTTGAGGTAGTCGGCCTGGCTGAGCACCCCGG
CCTTCAGGCCGCGCACCAGTCCCTCCAAGTAGCCATTGTCCACGTTAAAGTAAAGCTCCGGGAAG
AACGACATGGCTGCTGCGGGAGCGGCGGGACCGGAGAACCAGGACCGGCCGGCACGAATCGCG
ACTCCCCAGGTCAGCTGACGCTGCGTCCTCAGCGGGCTGTCAAGTGCGTCAGGTGACGCGCAAGC
GCCTCACGTGACCATAAGGGGGCGTGGCTCTGAGCGGTGCCCGGCTTCCGGGTGGCTGCGCGGA
AGCGCGGTGCAGGCGTGGGCTGGCCCTGCGGCGCCTGCGCGCTAAAAAGAGGAAGTTTGAGGCC
GGCCGCTTGGTGGCGCCTGTGGCCGCAGGGATTTGACTGGGCCTTCTCTAGCTTTCCCCGGTTTGC
TGCACTTGACGCTGCAGGGCGGGCGGGGTAAAGAAGGGAGCG
CGI: 39 range = chr17: 38089258-38089793
CGGGGGTTGGGGGCACGGAAGGGGAAGCTTCGGTCGCTAAGTGGGCCGGCCTTTGGAAGGGTCT
GGGAGGGGCCAGCGCTGACTAACAGTCGGTTTCCCTACCCTAGACGGCTGCGACGAGGAGCTGG
TGGGTCCCCTGTATGCACGCTCCCTGGGCGCCTCCTCCTACTACAGTCTCCTTACTGCGCCGAGAT
TCGCCAGGCTGCACGGTGAGCTCCGCGGAACATCAGCTGCCAACTGGCAGCGCACCGCGGAAGG
GTGGGGGCCTCCGGAGGACTTCGGGGAGAGGGATAGCCGGTTAAAGCTCCTGTCCTTTCTATAGG
CATAAGCGGGTGGTCACCACGGATTGGGGATCCGAATCCCTGGCTCCAGATAGACTTAATGAAG
AAGCACCGGATCCGGGCCGTGGCCACACAGGGCTCCTTTAATTCTTGGGACTGGGTCACACGTTA
CATGCTACTCTACGGCGACCGAGTGGACAGCTGGACACCGTTCTACCAGCGAGGGCACAACTCG
GTACTCTGGGCGCCAAGGCG
CGI: 69 range = chr17: 44074360-44075233
CGGCGCTGGCGCAGGCGGCCAGGAGAGAGCAGATGAACCGTTCGTTAGGAGCAGCGAGGTAGTC
GTGAGCGCTGAGATCCAGAGACTAGGACCCACTCCCTCTCTGAGCAGCAAATTGGGAAGAAGAT
GCTCACTCGGTAAGGGCGAGGGAGCCCGGCATGGCGCCCCACCACGGGCTCGGTCTATCTGCGC
GCCAAGATCCCGCTTGGGGCGAGGCGTTGGGTCAGCGTTTAGAGCCACTCCCTGCGCTGGTGGCT
GGACATAGCCTCCCTATCCCACCTCATCTTCCCCCATCCCCGACAGAGGAGGTTGTGAATCTACA
GGCCCTTGACGTTGAGGCGTCGGAGGGCGCACCTTTGTAATTGCGGCCTCCCTTCGCCCCTTAAGT
GCCGCTTCTGGGCGCCTAGGCTGGATATGAAAGCCCCGTTCCTAATCCTCTGCTCTGGTCCCCTCC
TCTGGACTGCTGGGACTCTAAGCTAGGCCCTCCCCAGGTTCCATCACTGCGGCGCCAACCCGCGG
CTGGGCTGTCCGCAAGAGGGAGTTGAAGGCGCGCGGAATCCCGAGGTGCAGCTGACCCTCCTCTC
AACGCCGACTCTGCCGCTCCCGCCCGGCCACCTCCCTGTCGGGCAGACTTCCTGTTCTCCTGCTCA
CAGCAGGGAGGCAGTCGCCGAGCCGGTCAGCAGCGTGCACGGAGATCTTCACTCTGCGCCCAGC
CCCGGGACACAGGTGCAGATCTCCAGCGGAGCACTGCGGAGTGCGCGCCGTCGAGCACTAGGGA
ATCCTAGACGGAGGACTTGGTCCATTCCACGCAGTCCCAGGCAGGTCCGCAGCGGAGGGACGCA
GCGGTCTCCAACTCCTGGTCACGACTTCGGCG
CGI: 269 range = chr17: 67627870-67631593
CGACTCGGCTGACGTTTTTGACCCGGCCAGGAGGCAAAGACCAAAACGTCAGAGCAGTAGCCCT
GTTACTGAGGAGCGTCGGCAGGGTCGCGGGTAGAGGGGGCTGGAGAATGACTTGTCAGAGCTCA
AGGTCGATGTGGCGCGGGGCGGCCTCGAGAGCGCCGGGCTCCTGCGTGGCCACGGCCGCCGCTG
CCAACCTTCGCGGGGACTTAGCTTTGCTTTCCATTGACTCCCTTTGCAAAAGCGCAGCAGAATCCT
GACCAGCCGCACCAGCCCCGGCGAACCCGAGCATGTTAATCTATTTATATGGATTATTACGGAGG
AACAGCGGGCGTTGAGTCACCAAAACATTTGCTTCAAAAGACTATTTCTAAGCACTTTTGCAGGC
AGGCAGGCTCGCTCCAGGCGCGTAAACTCGGCTACGCATTAAGAAGCGGCTGCTTTTCGAATACT
GCAAACTCCAGCTAAGTCCCCGGTGCCGCGGAGAGAGCAGTGAAAAGAAATGTCGGAGGTGGGG
GTAGATCCTAGTCTAGACACACACACTTGCGCGCACACACACACACACACACACAAGATTCGCGC
GGAGAAGGCACTAAAATTCTGGCATTCCGAGAGTACGACAAACTTACACACTTGGAAGTCCCGG
GTCCCCCGCCTTCCCCGCAGCACCCCCCGCCCCCCCACCCTACCGTCCGCCCTTTGGCTGCGATCC
CCTCCCCTCTCCTCCCCTCCCGCCTCGTCACCCAGCCCAGTGCCACAATCCTCCTCCCTCCCCAAA
ATCGGGTCCAATCAGCTGCCTGCCAACCCTGGGACTGCTGTGCTGTGATTGGCGGGTGGCTCTAA
GGTGAGGCGGAGTATTTATTAAAGAGACCCTGGGCTGGGAGTTGGAGAGCCGAAAGCGGAGCTC
GAAACTGACTGGAAACTTCAGTGGCGCGGAGACTCGCCAGTTTCAACCCCGGAAACTTTTCTTTG
CAGGAGGAGAAGAGAAGGGGTGCAAGCGCCCCCACTTTTGCTCTTTTTCCTCCCCTCCTCCTCCTC
TCCAATTCGCCTCCCCCCACTTGGAGCGGGCAGCTGTGAACTGGCCACCCCGCGCCTTCCTAAGT
GCTCGCCGCGGTAGCCGGCCGACGCGCCAGCTTCCCCGGGAGCCGCTTGCTCCGCATCCGGGCAG
CCGAGGGGAGAGGAGCCCGCGCCTCGAGTCCCCGAGCCGCCGCGGCTTCTCGCCTTTCCCGGCCA
CCAGCCCCCTGCCCCGGGCCCGCGTATGAATCTCCTGGACCCCTTCATGAAGATGACCGACGAGC
AGGAGAAGGGCCTGTCCGGCGCCCCCAGCCCCACCATGTCCGAGGACTCCGCGGGCTCGCCCTGC
CCGTCGGGCTCCGGCTCGGACACCGAGAACACGCGGCCCCAGGAGAACACGTTCCCCAAGGGCG
AGCCCGATCTGAAGAAGGAGAGCGAGGAGGACAAGTTCCCCGTGTGCATCCGCGAGGCGGTCAG
CCAGGTGCTCAAAGGCTACGACTGGACGCTGGTGCCCATGCCGGTGCGCGTCAACGGCTCCAGCA
AGAACAAGCCGCACGTCAAGCGGCCCATGAACGCCTTCATGGTGTGGGCGCAGGCGGCGCGCAG
GAAGCTCGCGGACCAGTACCCGCACTTGCACAACGCCGAGCTCAGCAAGACGCTGGGCAAGCTC
TGGAGGTAGGACCCGGCGGGGGCGGCGCGGCAGGGTGGGCATCGCGGCGGCTGGGGGCGCTGGT
CAGGGCTGATTTGCCCCGCCCCGCCTCCCATCGCCCGGGAGTTGCCGTTCCGGGAGCCGGCGGGA
TGGGGTTGGGAGTGGGAATGGGGTGTAACTGTGGCTCAGAGTTTGACAAAGTTCTTGGGCTGCTC
GCGGGGACGCGGAGGAGGGGGGTGGTAAGTGGAAGAGGTGAGGGAGGTAGCTGGAGGATGGAC
GAAGACTGGTGGGAGACGGAAGGAGGGGGCTGCCAGCCTGCTCTCCAGTCGCCTGGAAGCTCAA
TCGGGGCGGGGAAGTGAAACTTGCCTCCCTCCTACCCGGCCTCTTAAAACTGCACTCTCTCGTGC
AGCCCCACTGTCCACGGAGATGGGGCAAGGGAGAAACCGAGGTTGGAGGAGACCCTTGGCAGGA
ACTGGGAGGCGGGAGGAGGGAGGCTACTGGAAATAGGTGGGAGTGTATGGTGGGGGGTGAGAA
TTGGGGACCTTCTTGCAGCTTAAGTAATTTGGGGGAAAGTTTTCAAAGGGGGTTGGGGTTGGGGG
CGGTAAGTCGAGCAGCAAAGGCGTTTAGGGGGCAGCACCGGGAGTCGTTTTCATCTCCAGCGTTT
CCAAAATAGAAATAGAAGGGGAGGGGAGGGAGGGGGCGGGGAGTGACCGCTCAGGTCAGACTG
CAATAACTTATTTATTTATTTATTTTTAAGAAAAGTTATGAGCTGTGGTTGCAGGCAGGAGGGAA
GATGGAGTTGTGTGCAGAGGAAGCCGAGTGGTCTGGGTCGCCGCCTCCTCCCCGCCGACCTGACA
GTTTGGCGGATTTCACTGACCCCTCTCCCTCTTTTTCTCTGTGCCCCCCGCCCCGCCCCGAGCAGA
CTTCTGAACGAGAGCGAGAAGCGGCCCTTCGTGGAGGAGGCGGAGCGGCTGCGCGTGCAGCACA
AGAAGGACCACCCGGATTACAAGTACCAGCCGCGGCGGAGGAAGTCGGTGAAGAACGGGCAGG
CGGAGGCAGAGGAGGCCACGGAGCAGACGCACATCTCCCCCAACGCCATCTTCAAGGCGCTGCA
GGCCGACTCGCCACACTCCTCCTCCGGCATGAGCGAGGTGCACTCCCCCGGCGAGCACTCGGGTG
AGTCGCCCCTCGACCCCACCGGACAAGCTATCTCCGTCCCGCCTGGCACACCCCCTGCCCTCCGC
CTGGGAGATTCTTCGTGGGGACTTTATGCTTCCCGGGAGGGACACACTGCCCTTTGCGCCCGTCCC
GCTCCCCTCTCTACCCAGAGCCTAAGAGGCATCCAAACAACACACACACAAACACACACACCCC
AACTCAATCCCAGCATCCGAAGAGATTAACTTTTTTATTGGGAGGTAAAATGCCCTTAACAGCCT
TACAAGACCTCTCCCTTCTTCTCTGCTCCCCCACCCCAAAAGCACACACAGGGCTCTTACACAAGT
AGCAATTAGGTCTTCCGGACCCTCCGGGCCCCAGACCCTCCCCTGATAAAAGGGGGCTGTCCAGT
GTGTACCGGCGGGTTAATCATTGGGCGACTTATCTCCGGTGCAGCGCGCCTCTTGCGCGGGTGCG
GGCCCTTATTACACTTTAGCAGCGAGGGAGGGTCCCCGGAGGGTGCCTAAGACTAGGGCGTCTGC
ACAGCCCTTGTTGATTTTCTCGTGCTTGTTCTTTTATTGTCCACAGGGCAATCCCAGGGCCCACCG
ACCCCACCCACCACCCCCAAAACCGACGTGCAGCCGGGCAAGGCTGACCTGAAGCGAGAGGGGC
GCCCCTTGCCAGAGGGGGGCAGACAGCCCCCTATCGACTTCCGCGACGTGGACATCGGCGAGCT
GAGCAGCGACGTCATCTCCAACATCGAGACCTTCGATGTCAACGAGTTTGACCAGTACCTGCCGC
CCAACGGCCACCCGGGGGTGCCGGCCACGCACGGCCAGGTCACCTACACGGGCAGCTACGGCAT
CAGCAGCACCGCGGCCACCCCGGCGAGCGCGGGCCACG
CGI: 260 range = chr17: 71582000-71585125
CGCCTCCGCCCCGCGTTTCCTCCCCCCGATCTCCCAGGGGTCCTCTTTTGAGGGCACCCCAGCCCC
TCGACGGTCCCGGGATATCCTGCACCCCTGCCAGGCTGCAGGGAAGCGCCGAGGCGCGCGGAGT
GCGAGGTCCGTGCTCCCTGCGGCGCGGCGCACACGCAGGGTCCGGGGTCGACCCCTCCACCGCTC
CGGCGTCTGCCGGGGAAGTCAGTCCGCCTGGTCAGCCCAGAACCCCCGACTGCGGGGGCTGGAG
CTCGGAAGCAGGTACAAGCGCCACTCTCCGCCTGCGCCGTGGAATGCGCGCCGGGACCACTCCGC
AGCCCTTCCCCCAGCGCCGCCGGCCGCTGCTGGGGACAACCTCGCCCTCCTGTCTCTTGCTCCTCC
TCCTGACCCCAGCGCACCCCCATCCCCGCCCCAGATGAGGCAAGGCTCCCTCCGCCTTCAGCCCG
GCAGAGTCGCACTAGGAGTTGCAGCGGCCGCAGCCCCGGGAGCTTCCCGCTCGCGGAGACCCAG
ACGGCTGCAGGAGCCCGGGCAGCCTCGGGGTCAGCGGCACCATGAACGTCTCGGGCTGCCCAGG
GGCCGGGAACGCGAGCCAGGCGGGCGGCGGGGGAGGCTGGCACCCCGAGGCGGTCATCGTGCCC
CTGCTCTTCGCGCTCATCTTCCTCGTGGGCACCGTGGGCAACACGCTGGTGCTGGCGGTGCTGCTG
CGCGGCGGCCAGGCGGTCAGCACTACCAACCTGTTCATCCTTAACCTGGGCGTGGCCGACCTGTG
TTTCATCCTGTGCTGCGTGCCCTTCCAGGCCACCATCTACACCCTGGACGGCTGGGTGTTCGGCTC
GCTGCTGTGCAAGGCGGTGCACTTCCTCATCTTCCTCACCATGCACGCCAGCAGCTTCACGCTGGC
CGCCGTCTCCCTGGACAGGTGAGCCAGCGCCTTGGCCTCCCTGGGAGATGGGCATCCACGCGGGG
GATGGAGCGGGAGGCGGGACTGGGGACCAAGAAGGGACGCGCAGAGTGGGACAGGACACTAAG
AAGGCAGTGGAAGACAAGCGGGCGCGGAGGAGGAAAAAGAGGAATAAGAATGGGGGACCGTG
GTGTCCCTCGGTTAGATGCGTCCTGGGGCCTGGAAGCCTGGAGAATGTGGCTCTCCAGCGCCGCC
CGTGCCTGACAACGCGCAGCGTTTCCCAGTACGACGCGTTTGTGCGCGTTCATCTCGCTTGAGCTT
AATGCCCTCCGTGAGGGTGGGATAGGACAAAGTGCCCAATATACAGAAGAGTTGAGTTCCTAAG
TAACTCGCTCAGAGTCGCCAGCCAGGGATCGGGTGCGTGAAGTGACCGTCTGTCTCCTGCAGCCA
ACTTCAGGCGCCTCCACTGCGCTCGCCTCCAAGCCACGGTTTGGTTGGTTGGTGCAGCTGGCTCA
GGTCCAGGCTGTGGATCTTGGGTCCTTTGCAAGGATCCACTCCGGAGTCCCAGCGAGCGTGCCTA
AAGGTCCCTAGCTCAGTCCCAGCCCACTCTGCCTCTCGCCTCCAAACAAAACAAAACAAAATAAA
ATCCAAAACAAGTCGGGGCCGGGAGAGGAGCGTGCCCTGGGGTTCTTCCTCCCCAGCCAGAGGA
GAGCGAGAGACGCACATTCGGGAGAGCGCGGGACTCAGGTGGAGCTTGAAAGGACACTGGGAT
GGTTCCTGGGGAGGAAATCCGGGTATTTCCCCTCTCCATCCTCTGGAAAAACAGAGAGGCGAGGC
CAGACTGCCCCCACACCTCCTGTAGCCACTGAGCGCGAAGTGCGTTGGTTCCGAGCGCGCTGGTG
GGATCCACAAAGCTCGCATTCTCTCAGGAATCCCCTGAGAAATTAACTGTCCCTTGCCCAACATG
TCTTCTCCAGGCTGTCTGCTAGAGCCTCAGGCGCCTCCGCCCTCCCTCCCGCGGCACCGTCACCAG
TGGGTAGTCACAGCCTCCCGGAGCCCATAGCCGGTTCTCCAACCTTTAGTCTTCAGTGGCTTTGGG
GTGCCCTCTCAGTGGAGACTGTGGTTGCAGTCCCCGGGGGCAGCGGGAGAATGGCTTGAAGGCA
CACCTTTCCTGCTGCCGGGCCCGCCCCATTTCCAGGCTCCGCTGAGTGTCTGGGACACGCTGGGA
GGCCCCCACCTCCGCCCTCACGCCGAGCCTCACCCCCACCTCCTCTGTGTGCGGTGTAACCATGCG
CTAAGGACCTTCCTCGAGAGCAGCCTTGGGACCGAGGTGCAGGGGTCGCGGCCCTCCAGCATGA
ATGTGCCCGCTCAGCCGACGTCTCCCTTCCCGGTCTGACCGCAGGTATCTGGCCATCCGCTACCCG
CTGCACTCCCGCGAGCTGCGCACGCCTCGAAACGCGCTGGCAGCCATCGGGCTCATCTGGGGGCT
GTCGCTGCTCTTCTCCGGGCCCTACCTGAGCTACTACCGCCAGTCGCAGCTGGCCAACCTGACCGT
GTGCCATCCCGCGTGGAGCGCCCCTCGCCGCCGCGCCATGGACATCTGCACCTTCGTCTTCAGCT
ACCTGCTTCCTGTGCTGGTTCTCGGCCTGACCTACGCGCGCACCTTGCGCTACCTCTGGCGCGCCG
TCGACCCGGTGGCCGCGGGCTCGGGTGCCCGGCGCGCCAAGCGCAAGGTGACACGCATGATCCT
CATCGTGGCCGCGCTCTTCTGCCTCTGCTGGATGCCCCACCACGCGCTCATCCTCTGCGTGTGGTT
CGGCCAGTTCCCGCTCACGCGCGCCACTTATGCGCTTCGCATCCTCTCGCACCTGGTCTCCTACGC
CAACTCCTGCGTCAACCCCATCGTTTACGCGCTGGTCTCCAAGCACTTCCGCAAAGGCTTCCGCAC
GATCTGCGCGGGCCTGCTGGGCCGTGCCCCAGGCCGAGCCTCGGGCCGTGTGTGCGCTGCCGCGC
GGGGCACCCACAGTGGCAGCGTGTTGGAGCGCGAGTCCAGCGACCTGTTGCACATGAGCGAGGC
GGCGGGGGCCCTTCGTCCCTGCCCCGGCGCTTCCCAGCCATGCATCCTCGAGCCCTGTCCTGGCCC
GTCCTGGCAGGGCCCAAAGGCAGGCGACAGCATCCTGACGGTTGATGTGGCCTGAAAGCACTTA
GCGGGCGCG
CGI: 38 range = chr17: 75936057-75936582
CGAGAGCCTCTACGACGCACTCAACCAGTACTACGTCCACCTCGGCGGCCAGAAGTACGTGGACC
TCGGTCTGGGGACCCACCGCGTCAAATGTCGGGTTCACCCCAACTTCCGCCTGATTGTCATTGAA
GAGAAAGACGTCGTGTACAAACACTTTCCCATCCCCCTCATTAACCGGCTGGAGAAGCACTATCT
GGATATCAACACGGTGCTGGAGAAATGGCAGAAGAGCATCGTGGAGGAGCTCTGTGCGTGGGTG
GAGAAGTTCATCAATGTCAAAGCACATCATTTCCAGAAGAGGCACAAATACAGCCCCTCTGACGT
CTTCATCGGCTACCACTCGGACGCCTGCGCGTCTGTGGTGCTGCAGGTCATAGAGAGGCAGGGTC
CCCGGGCCTTGACGGAGGAACTTCACCAGAAGGTGTCTGAGGAGGCCAAATCGATCCTGCTGAA
CTGCGCTACGCCCGATGCCGTGGTCCGGCTGAGCGCCTACTCGCTGGGCGGGTTCGCAGCGGAGT
GGCTGTCG
CGI: 22 range = chr17: 76404186-76404427
CGTCACGTGCAGAGCATCCTCCCGACTGGCGAGGTGGCTTCCACGTGCCCCGTCAATCAGGGCTG
GACAGTCCGTCACGTGCAGAGCATCCTCCCGACTGGCGAGGTGGCTTCCACGTGCCCCGTCAATC
AGGGCTGGACAGTCCATCACGTGCAGAGCATCCTCCCGACTGGCGAGGTGGCTTCCACGGAGCG
ATTCTCGAGACCCGAGGGGCGTGGACACAGCTTCTACGCAGGCAGTCG
CGI: 53 range = chr17: 77537903-77538626
CGCGCGTCGGGCGCTGGGAAGGTGACTGTGCGTTGGGCGCGCTTGGTGGCCCCAGGGCTGGGCCT
GCTCCCGTCTGACAGTAGTGCCGCGCTGCATGGCACGGGTGTTGCGTTCGTGGGTGAGAGCGCTG
AGGTCTGCACGTCGGGTCTCAGTGGTCATGGGGATGGAAGCCACCCCAGTGGCTGTCGGGGTGA
ATGGAGGCCCCTGGAGATGGCAGCGGGTTAAGCACGAGCACGGAATTCTCCCTCCCAAAACGGA
ACTCAGACAACAGTGACGGAGCCTAAGAGCAAAGCCAAAGCCACGGGCAGGGGTGAGCGGGAC
CCGAGGCAGGAGATGACGGAGCCTAAGAGCAAAGCGAAGCCACGGGCAGGGGTGAGCGGGACC
CGAGGCAGGAGGTGACAGAGCCTAAGAGCAAAGCGAAGCCACGGGCAGGGGTGAGCGGGACCC
GAGGCAGGAGATGACGGAGCCTGAGAGCAAAGCGAAGCCACGGGCAGGGGTGAGCGGGACCCG
AGCCAGGAGTGGCGCCGGGCAGGGAGGCAGGCAGCCCCAGGCGCTGTGTGGGCACTGGGTGAG
AAGCAGGGATCCCGGCGCCCTGGCTCTGTGCTGCTTCCTGAGACGAGAGTGGCAGAGGGCAAGA
AGAAAACCAAACAGCGCGGGGAACAGAAGTCAGGAAAGATGAACGTCTGGAAGGAAATGCCCC
TGGGCCGGGCGGTGCCCCCGGGCG
CGI: 330 range = chr18: 894579-899574
CGGAGAGACCTCGGAGCAGAGAAGGCGCCGCCGACCCTCGCGGCTGCCTGGCCCGCGGCTCCTA
CAAAGGCGGGCTAGCCGCCCGCCCTCTCCCTTGCCTTCCTCCCCTTCTTTTCTGACTTTCCCTCTTT
CCCTTAATCGCCTGCTTCTTCCTCCGGGTGGACTTACGGCCACCTTGCTCCTCCGCGCTTCACCTC
ATCGCCCCCTCTTTCTTTCTTCTGCCTCTCTCTCTGCGCCCCCTTCTCTCCGTGTCACGCTCCCTCCT
GGTTCTGCGCGTCTACAAACTTTTGAGCAGAACACGAGCCTCGGCAAACGAGTCCCGCAGCTCCT
CCTGCTGCTCCCGCTGGTTCCTGCGGCTTCTGCTCAGACACCAACGCCAGACGGCGATGCCTCTCG
GGTGGTGACTCCAGCGCAGGAACTTGAAGAAGCGCTTTGCCCGCCGTCCTACCTGGCAGCTCTCC
TGGCAGCGGGAGGAGTTGAAGGGTAAGGGAGGGAAAATCTTACCAAAGCGACCGGCTCACTCGA
CTGCTGATTCTTTCGCTTGGCATCGCGTCAGGGGAGTTAGCTTTCCTTCAGCCGGGTCTGGCTAGT
TATTGGGCGCCGGGTAGATGCATATATATATATTTTTTTCTAACTATAGCAAGCAAGAAGTGGCA
GGGCGCGCACCGGCTGTCGCCAAGTGCTGTTCAACTCAGGGAGCCGGGGCTTCGCTCCGTCCCTC
CCCCGGCTTCCAGAGCTTTTTGGGGTTGGAGGGTGGGAGGCCAGGGGCGTTCTCACAGCTGTGTG
TCCTCTTTCCCATCCTGCGCAGAATGACCATGTGTAGCGGAGCGAGGCTGGCCCTGCTGGTCTAT
GGGATAATCATGCACAGCAGCGTCTACAGCTCACCTGCCGCCGCCGGACTCCGGTTCCCCGGGAT
CAGGTAGGTGCTGGCTGCCTGGCCCAAGCAGGAGCTGGGGCTTCCCAGGCACAGACGCTTCCTCA
CGGTCTCCTTCCTGCAGTCCTTTGGGTCCAGACTACTAGCATCGCCCTCTGCGCCCCCGGTGCGCC
TCCGCCAGCCTCGGCTGGACAGCGGGTCCCCATTCTAGCCGAGGGTCTGGCAGGCTCCGCGACTG
CTCGGACGCCTCCCCCAGCCCTAGGCAGCTCAGGGTCCCGGGTAGAGCCAGTGAGCTTCTGGCCG
CTGGAGAACCCCCCCTCCCCCAACCCGGCCCACAGGATGGGGGCAGGGCACGGCCCCTAGCTTG
GTTTCTTTTACCTATTCTTGGGACGAGTTAGGAGAACTTCAGCTCTGGAGCCTGGCCGGGGGTTGA
GCGTGAAGCTCCCTCGGACTTTGCTTTGTTACTGCTTGTTCTGGACTATCCGGGTGGGGTCTCTCT
CTCTCCTCCACCCTTTCTTTTCATTTCATTCCAATTCTTTCCCCTGAAGAGCTTTCTTTCAAGTGATC
CGTGTTCCAACTGCATTTTGAATCCCAGGCTGTCTTGGGGGGCGTGCGGTGGGGAGGGTGTTGGC
CCGGTGTGATTGAGGAAAAGCGACTTAAGAGAGGGAAGAACAAGGACGAGACTGCGAAGGAGG
GGGAAAAACAGGCGCAAAGGAGGAGGAAGGGAAAGCCAGCAGGCAGGCAGGACCGGGAGAGC
AGCCCTGCCTGGCCCGGGATGGAGGAACCTTGGCTTTTTTCTTAACCCCGGGTTTCTAACCGGCA
GGCGCGGCCCAGGTTCCCGGAGGCAGCCCCAGAGTCGCGGACCGATGTGCCAGGCTGTGGATGA
GCCCTGGGTAGGGGAGGGTTCGTACCAGCGGCGCCTGGGGCAGCGAGGAGCGCGCGTTCTGCCT
GCGAAGCTGCCTTCTCCGAGCCCCGCCCAGGAACATTAGCTCTGGGGGGCCGCTGATCATTGATT
TGGACGGAGAGATGGGTTCTGGGTTCTGTATTAGGATTCCAGCATCTGGGCTCGAGGCAGGGCAA
TATCCAGAAAGACCCCAGGGTTCGGGGTACCCGGGCCAGGGCTGAGGCGCATCGCCGAGCAAAG
GCTGGGTGCGAGGCGTGCGGAATGATGCGCTTGCCTTGCCCGGGCCTCTCCAAGGATGGAGAAA
AGGCGAGTGAAGCAGCGAAGTACGACTCCAACCCCGCCCAGAGAGTGCTACTAGCGCTGGCTGC
ACGCCAAGTCTCTCCAGGGGTCCAAAGCGAGAGGGATTTGTTTTAACCCATCTCTACCCGTCCTG
TGTCAAGAACGGAGGCTGTAGAGGGCGACTGCGAAGTCGCCAGGCACTCGCTGGATCTCGGTCC
CCCTCCTCGTGCTCTGGGGTTGAGATGGGGCACCGCCATCGATAACAGATCAGCGCGAACTATTC
GTTTAGTGGCCTTAAAACACCCTGGTTTCACCCTCAGCTATTTTCAAGTTCCCGTGTGCCTGGCAC
TTTCTCCGTGCGAGAAGCACCGGAGGGTGCGGACGCGCCACAGTCTGAGCCGCCGCCGAACTGG
CTAAGTTTAGGGGCATTTATTATTCATGTTCCTGCCAGATCCTCGCCTGCCCAAAATAGAAACCGA
GGTTCTCCGTGACCTACATCTGCTCGGAGAAGGGCTCCCCTGGGCTCGGAGGCTGGGGTGGGGGT
GGCTGAGGAGTTGGCCCCCGCACGCCCCACGCATCCTCTCCTTTGCTTTCTGGGCCTCCCCATTCG
GGTCTTCGCGTGGGTCAGCGCCCGGTCTCCCAGGGCCTTTCTCGTCCCCGCCCGTTGCTGCTTTGG
GGAGGCTCGGGAGCCAGGCGGGGAGGGGGGCGGTCCTTTTCCGTAGACAGGTGTGCGCGATCGG
CGGAGACGCCTCGGTTTCCCAGCGCTTGTTGAGGCCGTGGCCCGCAGGACGACCCTTTACCCGCG
AAGGGGGGGTGGGCGGGCCGCCCGGCGGGGTAGGAGTGGTTGGGTGTCGTTGCCTCCTCCTTACC
TCTGCTCCCACCCCCAGTCCTGGGAGAAGAGACAATTCTCAGCGGAGGACTTTTATCACCTGTGA
AAATCCGCGCGAGCCCCTTACTTTGGATCCTCGCCGAGCTGGGGAGGAACTTGCACTGACCACAC
CTTCTGTCCCCGGCCACCCCGCAGGCCAGAGGAAGAGGCGTACGGCGAGGACGGAAACCCGCTG
CCAGACTTCGATGGCTCGGAGCCGCCGGGCGCAGGGAGCCCCGCCTCCGCGCCGCGCGCCGCCG
CCGCCTGGTACCGCCCGGCCGGGAGAAGGTGAGATTCGCGCGGCCTCGCGCACACCCGCGGCTG
GGAGCTCGGGACTGCGGTGACGGGAGGGGCAGTGTGGTGACCCACCCAGGATTTTTTTTTTTTTC
CCGTGAAAGTCCTCAAGCCTGTCCTCTCCCTGGCCCGATCCTATTGCAGCGACAGAAAATCAGCA
GCGGGCGGGTCTGTGTGGACCTGAGGGCCGCGTGGGGACCGAGGGGGGCTGTGGCCCAAAGAGT
GGCAGTGAGTGGCGTCAAGGAACCCACACTCCGCATCTGCCACTCCTAGAGCCGGGACTAGCTCC
CGATCCTAGCAGTTGCTCTCGAGATCATCCCGGGAGTTATTGGCGAGTTCTGGGCCTCTGGAGGT
TTCCCTGTCAGCCTCCCCGGCCGCCGAGGGGGCGCGCGCCCAACAAGGGGGTCTCTAGCGGCCAC
CTGGGGACAGAAACAGTGACCCTGGGCGCGCACTTTGCCTCCCCGTTAGAGATGTCGCCCACGGG
ATCCTTAACGAGGCCTACCGCAAAGTGCTGGACCAGCTGTCCGCCGGGAAGCACCTGCAGTCGCT
CGTGGCCCGGGGCGTGGGGTAAGAGTTTGTGGAAGGATTAACCTGCGCGCGCCGGGGTGGGTGC
CTGTGCGGGGCGCGCGGGGCGGGCGGCGGTGGGTGCCCGTGGGGGCCAGGGTGAGTCTGCGCCC
CTGGGTCTGGGGTGGGCATCCGCCACGGGTCGCAGTTGGAGATTTTGAAGTGGCACTTTAAATTT
GCCCAGAGAGCTCTGGAAGAGGCAAAAAGGGAACGCGAGCCAGGGAGTTTGATCCGTTTTGAAT
GAAAAGAAAGAGAAACCAAACCAAACCTCTCAGTCATCCAAAACCTTCAGGCTTCCAGGGAGGT
TTTGCTATAATTTTCTCTAAGCATGACTGTTTCTGGGGGAGGGGAAAGGGGTGGTTGTATTTACTG
AAAATTCAAATCGAAATAATAAATGGCCAAATGTGGACACTTATGGACCCAAACAGTTTTGCTCA
CGCCAGAGAAACTGAGAGCACAGGGCTTGTGTGAAGCCTATCTCGGCAGAAGGCAACATTCTAA
TAAAGCCCGTGGGAAAACAGATTACATTTTCGCCATGAATAAGTCATGCAGTGAAAAATATTGCC
TACAGCCTGTCGACTTATATTATTATCACGTTTTTCAACTCGGCGTGAGGAGGGAGAGGAGTGTT
CATATTTGACTAGGAATTGCAGGATCGATGCAAACTCCAGGGCAGCAGCCAGACTGGCATATGTA
GGGCTCTCCGGTTACTTTCTCTGTATGTCGCGGGTGAGAGGAACAGCGAGGACAATTTAGCGCAA
ACACACGAAGGGTCGGATCTCAAGGGGGCAGCGCTGGGAGAAAGGTTAGGCTTGAAGCGCGCGT
CGCCTGCCCGGATCTTATCCCGGGCCCCCTCCGCAGGGTTTGGTGCCAGGAGATCCTGCGTGGGG
AGGGGGGCATCGAGGGGCTGCCGTCTCGGCCCTCCCCACGGCTGCTTCCAGGCAGAGGCGGGCG
ACGCGGTGGGCAGTGCGAGCCCCGGGCCCTCCCCGAAGGCTCCCGCGTGGGGTGGGGCCCGCCT
GCTCCCCGCGGCGATTGAACCTGTGTCTCCCGCCCCGCCACCCTCTTCCCGACCCCTTTGCTTGCA
GTGGGAGCCTCGGCGGCGGCGCGGGGGACGACGCGGAGCCGCTCTCCAAGCGCCACTCGGACGG
GATCTTCACGGACAGCTACAGCCGCTACCGGAAACAAATGGCTGTCAAGAAATACTTGGCGGCC
G
CGI: 338 range-chr18: 74837994-74842232
CGCAGAAATTACTCGTGCGCACCATTTCCGCTGTGGGGGCATTCGTACAAGTTTCCGCTGCACAC
ACAGCCTCCCGGGCCCTCTCCTCCAAGGCTCTGCCGGATCTTCCAACGAAATCCCAGAGCAGCCT
GCGCTGGGGAGCCCGCAAGTCTCTCCAGATCTCTGCACCCCGCACCGCCCGGAATCTGGGACGGC
GCCCACGCAGGGCTGGGCCAAGGGCAGAGCTCGCACCCTGCCTTCACGCCCGGTTCACTTGCGTC
CACGAAAGCAGCGTGCCGGCCTCCTCCATCTTCCCACTCGCGCAACGCACGGCGACCCGCGCGAC
ACTTCTGCAATCTGAAGGCTTGCTTCTTACAAATAAAGGGCCAGAGTCTCACACTTGCCTTCGTTG
GAGGGACTTAGAAGATCCTCCCCACGTCCACACCTTGTAGGAAATGCAAAACAGATCGATGAAA
TTAAACAGTTGCATTTGGAAGCCCCAGAAAGACCTAAAGACATCGTGCCGGTTTGTTGGAGAGAG
GGTTGCGGGACAGGGGGAGCGGGCCTTACGCAACAGAAAAGGTGGGCACAGCGCGCTCAAAAT
GACCCAGTGAGGAGTTGGTGCCGCCGGGCCAGAGGCTGCGAGTCCAGCTGGCTCTGGACTTGCTC
CGCAGGCGTCAGACGCCGTGGGAACCTGTGTCTGCTTCTTCTCTCCAAAGTGTATCGGTTAAAAA
AAAATAAAAGTAGTAGTAGTAGTAGTGGTAAGGAAAAAAATAAAAATAAAAAGGAGACACAAT
TAACCAGGTCATAAAAGCTAGGGCACCTTCGACCAGGGCTCTGGCCCTCCAGCGATCGTTTTGCG
TTGTTTCTCTTCTCAAAAGTAGTCTCAGACCCCTGCCTTTCCGCTGCAGCTCTGCGACTTCCCCAA
ACTCCTTAATCCTGTAAATTCTGCAAGAAACTCCCATCCTGCAAGCTGCTTTTCCCCCTCCCCCCT
GCGTTCCTTTTTTCTCTCCCCACCCGCGCCGCCTCTCTATGCCCCTCTCTTCTCAGAAAAATTCCTG
CCCCCCGCGCGCCCCAAAGCCCGGGCTGCAAACTTTTCCCCGCCGGGCGCCTCTGCGCCAGATGC
CGGAGCGTCTCCACAAAGCCTGAGCATCTGCACAAGTTCGCAGCCTAACTGCGGGATAAAGACG
TTTCCCCCGTAGCTTAACTAGAAAAGCGCCATCGATGGGTGTGTTAAACGGGATAACTAGAGATT
TCAAACACCTTTTATTTGCCTGTCTTGAAAAAAAAATCTAAATGAATACGCCCGCTACCAAAAGG
CAAAATAAAACCAACCTTAAGGGTTTTTGTTGTTTTTTTTTTTTTTCAAAAGTGGCGATAGGGACT
GTTTGGACCTGACTCCAACCTGCGCCCTCCCTTCCTCTATGACCCTCCTGCGCTTTTCCTGGAACC
CAAAGCTCTGACTTCGTCAAACTTACACAATTAAAGGCAGGCGGAAGAACGCGGGCTGGGAAGC
AAGCGGGAAGATTCTAGAATGGAAGGGAGCCCGCCGAGCGCCGCGAGCCGCGCCAGGCCGGGTC
CGATGGAGCAGGCGGGGATTCCTCCCCCAGGCGGACCCCCGCCACCAGCCCTGCCGGGAGCTCG
CGGCCTGCGGAGCGCCCGGGCTGGCCGCTCACCGCCCGCTTCCCCCAGCGAACGACTCGGGGAA
GCTCCAGGAGGCCATCTGTGCTGACGGTTCACACCAGACAGGACCACTTGCAAGGACAAAAATA
AGAAATTTAGGAAACGAAAAAAGACGTACTGGGGCGAGGGGCGCGGGCGCGGCGACGACGGGG
CCGGGGGCACATCCTGGCGGCCGCTCGGGGAGAGAGGACACGCGCGGGAAGGAGCGCGGCGGG
TGCACGGCCGCGGGTGGGAGTACGCGCCTGTGCGCGCGGGGCGAGGGCGAGGGCGCGTGCGTGT
GACCGCGGGGAGGGGGCGGGCGCGTGTGCGGGGAGCGCGCCGCGCCAGGGGCCGAGTGTGTGG
GGCCGATCCAGAAGTGCGCAGCCCCCTCACCTGGCCCCCGTGTCATCCCCGAAATCCCGGGAAAG
GGTGGGCCGCGCGCGGGAGTTTGGTGGAGTTGGAACTTTCGGTCGCGCTCGCTGCCCACTCCGCT
GGCGCCCGGTGGCCCGTGGTGAAGGGGGACTAGGGTGGGGAACACCGGGGCCCTGCGGTCCCCT
CCCTTTCCTGTATTTAAGAAGCCGCCGGCGGCGCAGAGGCCCAGGCGGGCTGGCGCGGGGGCGA
GGCGGCCCGGTGGCAGCAGCGGGCGGGGGGGGCGCTCCGGAGTCGGTGGGGCCCGCGGGTTGGG
GGGCGGGGAGAGGGGGGAGTGGAAGGGAGGGGGAACGCAGGGGAGGGAGAGGAGGGGAGGAG
CCGCGCGGCCCGCGCCGCTTCCGAACCGGAAAGTTGGTCTTGCCGAAGTCCTGCCACCCCGGCGT
GCGCACTCCGCTCCGCTCCGGCCGCGAGCCTCCGAGCCCGGCCGGCCGCCGGGGGAAGCCCGCG
GAGGGGACGCGGGGCCGGGCGAGAAGGTCCGGAGAGCGGGGGGCACCTGAGCCCGGGCGGGCC
CGCCGCGCTGAGCGGCGCTGAGAGCCGCGGCGGAGCAGCGAAGGCGGCCGGCCGACCCCGCGCG
CCCGGAACAGGAGGCGCGGCGCCCGAGCGGCCCGGGCGAGACAAAGGCGCCGGGTCGGAGCCC
TGCCCGCGGCCGCTCGCTCCGGGAGGGGCCGCCCGGCGGCGGCGGCGGGGGGGGCGCGGGCGGC
GGCGCAGACACTCTATAAAGGGGCGAGCCCGGCGCGCCGGCGGAGACGGCGCCGCGCGGACGCC
GCCAAAGTTTGCTGCCTGCGCCCTGCGGAGGGACGGCCACCGCGGCCCGCGCCGCACCCGGGCC
CCGCCACAGCCGCACCCGGGGCGGCCGAGGAGCGCGGCGCCGGAGCCCGCGATGTGAGGCGGCG
CCGGGCAGCGCGCGCCCCGGTCCCGAGGCGCCGCGGCCCCCTCCTCGTCGGCGCGGCCGCTAATT
GCGAGCGCGGCCTCATTTGCATAGGCCGCCGGAGTCCGCTGGAGCCCGGCCAATCGGCGCGGCC
CTCCGCTAATGGCCATGCATTATTCACCAGCCTAATTGCTCAGCCCCATGCGCGGCCCGCGCAGC
CGCCGCCGCCCCGCGCCCCGCGCCGCGCGCCCGCCAGGCCGCCCCGCGCCGTCCCCGCCGGCCGC
CCCGCTGATGCCGCTGCCCCGCGCGGGGCCCGAGCGCCGCTAGCAGCATGTCTCGGCGCAAGCA
GGCCAAGCCCCAGCACCTCAAGTCGGACGAGGAGCTGCTGCCGCCTGACGGGGCTCCCGAGCAC
GGTGAGGGCCGGGGCTGCGGGGTGGCCGGGGGGTCTGGGGCTGCCCGTCCGGGCTGGGGAAGCG
CGTGCGGCGGGAGCGGATGCGCGCGTCCGGGAGCGGGAGAAAGTTCCCTGCTTCCTGCGGGCAA
GCGTCCGCCCCGCGCCAGGCCGGCCGCGGGGCCCCGGGTACTTCGCCGGAGCGCGCGCGGCCGC
CGAGAGAGTTGTGGGCGAAGTAAACTTGGCTCCTCTCCTCGGAGTCGGGGAGCTGCCCGCGAAG
GGCGCCGAGGCCGCGGCCGGCTCGAGGACGGCTCGGAGGCCGGGGCGGGAGGGAGTCCACGGT
GCCTCCGCCGCCGCGCCGCCCCCCAGGGTCTCTGCGCCAGGACGCTGAGGCCGGCGGCGGCGGG
GAAGGCGACCGCAGCCCACCTACCGCTGGACGCGGGTTGGGGACCCCGCCGCCCGGCCAGCTTT
GTTCGGGGGCCCGCGGCCCCTCCCGGGCCCCCGCACCGCCTCGGGTGACCCGCGGTGTCCCAGCG
CGTTGACGCAGCCTGTGATCCCTCGCGAGGCGAGGAGAAGGTCGGGGGCTTGGCTCTGCCTAATG
GCCGCCCGGGGAATTAAGCTGGGGGTGAGCGCAGCGGCGGCGGCCTGGGCCTGGCCCCTGCTCG
CGGCGTGTTTCCGGGGCGTTCGTTGCAGCGTCTGCGCGGGCCTTTTCTCTCCCGTCTTTTTGGATC
CGCCGAGGCCGGGCGCTGGAGACCTCGGCTTTGCAGTCATTTCGCTGGTAGGAGCGTCCTCTTCG
AAACATCCAAGAGCAAAGGGCAGGCGCCGCGAAAGTTAAGAGACTGGCAAAGGGCTGGACTTCC
CAGAGTGGCGCCTTAGCCCCGCAAAGTTTGGGGCGCCCCCACCCCCTTCGTCG
CGI: 43 range-chr18: 75565313-75565933
CGGGTGCAAACGCATCCTCTGTCCCACGTGGAGTGGTGTGTCCACACGCATGTCCTCTGTCCCAT
GCCCACGTGGAGGGGCGTGTACACACGCATGTCCTCTGTCCCGTGTGGAGGGGCGTGTACACGCA
CACGTGTCCTCTGTCCCGTGTGGAGGGGCGTGTACATGCTCACATGTGTCCTCTGTCCCGTGTGGA
GGGGCGTGTACATGCAAACGTGTATCCTCTGTCCCATGTGGAGGGATGGGCACATGTGTGTCTTC
AGGACGCCTGTCTCAGGCACCCGTGTGTCTTCAGGACGCCTGTCGGGCACACGCGTGTCTTCAGA
ACGCTTGTCTGGGGCACACGTGTGTTCTCAGGATGCCTGTCTCGGGCACACAGGTTGCGGGGGCG
GCCGGGGCTCCTCAGGCCTTTTCTCCCGACTGTTGCTACTATTCTTTTTAGGGCATCTTTCACTTCC
AGCTGGGCCGGGGTGAGCCCATGCTGCACACGCGCCTGCGTCCACACTGCAAGGACTTCCTGGAG
AAGATCGCCAAGCTGTACGAGCTGCACGTCTTCACCTTCGGCAGCCGGCTGTACGCACACACCAT
CGCAGGTCAGTCAAGCCGCAGCCGAAGAGGCCG
CGI: 33 range = chr19: 898548-898981
CGGGACCGTTTCACAGATGAGAAGACCGAGGTGCCAGGGTGGCGATCGCACGAAGGGCCCGTCA
CGGGAGAATGAGGAGGGTCTGGCGTCAGCAAGCTCCCCCGGAATCTCGTGATCTGGAAATCCAC
AGAGAAGCACCTTCACCACGCCAGCTTCCCGGGCCGCGCTTCATTGTCATTTCTGGAGAGTGTTA
TTAATATCCGCCCCGTGGGTGCGGCGCTGTGTATTTCCAGAGAGGGGACTCGGGGCCCATCAGGG
CCGGGGGAGTGGAGCCGGCAGATGTTCGGCCCTGCCCAGAATCACTTCCCCAATTTCCCCACGCC
CGGCGGGGCTCTCAGCATCTGCATCCGCCGAGGCCTGGCTGTGCTGACGGGAATTGAAAGGACCT
CGTGGCTTAGGCAGGCGGGGGAGCCCCCCGGCTGGTCAGGTGCACG
CGI: 70 range-chr19: 9131907-9132918
CGCCGCGACTATGGGGTAGCGTTCAAGGGCAGGCCGCACCTCACTCAGCACATGAGCATGTACG
ACGGGAGAAAAATGCATGAATGTCATCAGTGCCAAAAAGCCTTCACCACGAGCGCGTCCCTCAC
ACGGCACAGGAGAATCCACACCGGGGAGAAGCCTTACGAGTGCAGCGACTGCGGGAAAGCCTTC
AACGACCCTTCAGCCCTTAGGAGCCACGCAAGAACTCACCTCAAAGAGAAGCCCTTTGACTGCAG
TCAGTGTGGAAATGCATTCCGGACCCTCTCGGCCCTGAAAATCCACATGCGAGTTCACACTGGCG
AGAGGCCTTACAAGTGTGATCAGTGCGGGAAGGCTTACGGCCGGAGCTGCCACCTCATCGCACA
CAAGAGAACGCACACCGGAGAGAGGCCCTACGAGTGTCACGACTGTGGGAAAGCTTTCCAGCAC
CCCTCCCACCTCAAAGAGCACGTGAGGAATCACACGGGGGAGAAGCCCTACGCGTGCACGCAGT
GCGGCAAAGCCTTCCGCTGGAAGTCCAACTTTAATTTGCACAAGAAGAACCACATGGTGGAGAA
GACCTACGAATGTAAAGAATGCGGGAAATCCTTTGGCGATCTCGTGTCCCGGAGGAAACACATG
AGGATTCACATCGTCAAGAAACCCGTGGAATGTCGGCAGTGCGGGAAGACCTTCCGAAACCAGT
CCATCCTTAAGACTCACATGAACTCTCACACTGGAGAGAAACCATACGGGTGCGATCTCTGCGGG
AAAGCTTTCAGCGCGAGTTCAAACCTCACCGCACACAGGAAGATACACACGCAAGAGAGACGCT
ACGAATGCGCCGCCTGCGGGAAAGTCTTCGGTGACTATTTATCCCGGCGGAGGCACATGAGCGTT
CACCTTGTAAAGAAACGAGTTGAGTGTAGGCAGTGTGGCAAGGCCTTCAGGAACCAGTCAACGC
TGAAGACGCACATGCGAAGCCACACGGGGGAGAAACCGTACGAATGCG
CGI: 25 range = chr19: 12985960-12986259
CGGAAAGCGCGCTTTTCGGCAGAGCTGGTTAGTTTTTAATGCGGGCTTTTTTTTTCTCACCCGCCG
TTCCCTCCGCTCCGGTCTGATTCGCAAATCCCCAAACTGGCACAAACCGGGAAACTTGCGGCCGG
CCGTTCTTCTCCCCGAGTTGGAGACAGCCCTCTCCTCTTCCCTCCCCCGCCTCTGGGCCCTGACCC
ACTGCCTGGTTTGCGCTAGAGCCGTTTAAAAAAGAAAGAAAGGAAGAAAGGAGCCGGGCTCTGG
TACCCCGATGCCAGCCGAGGGCGCGTTTCCCAGGCGGCG
CGI: 35 range = chr19: 55553586-55553895
CGCGACCACCCGCTGCGCATGCGCTTCACTTGGGCGTCACCCGGGGAACTGCGCCTGCGCAGTCT
CTCCCCATCCGAGGTCCGCTCCGCGAGTGCGAGCGCGCGCCAGGCCCACCCGGGCGCTGCTCTTC
ATGTCCCCGCGGTCGAAGACGGCCACATACGTCCCCAAGAAGACGTCACCGAGGATCCAGAAGG
GCCCTGCAGGCGGAGGGACATCCAGGGCCTGGAAACCGGACAAGCAGAGGCGGACGCCATTTCG
AGTAGTCTGCAAGGCAACAAGACGACAACTGGGTGTCGCGGCCACAAGGACG
CGI: 47 range-chr19: 57082654-57083180
CGATTCGCTTCCCCACCACGACGCCCTAGCGCTACTGTGCAACGAAGACCTCCCAAGCACTGGTT
CCAATGCGGAGACCATGGGCTCCCAGACTCTGGGAACTCCAACACGACTGCGAAACGAACTCCG
AGCGAGGACTCCCCGAGAGCTCCCCGCAACACGGACCTCACGCGCTAGCGAACAACAGAAAAAA
AAAAGCGCGCTCTCCCTGCCCCTGAAACATTCCCAGAAGCCCACGCAGACCAGACCGATGACCTG
TCTCCACTGCTGGAGGCGAGTCAGGGACCCGAAGTCTCTAAACACTCGCCTCTACCCGCCGCCCC
GCGAACCCCACACACTGCAGACGCGACACTCGCAAGTTTCGGGGATGGCGGCCGGCGAGGGCCA
TACTGCGTCTTTCCGGAGACACGGAATACGGCACCAGCCGTCCCTTTATGATGCAATATGTCTGC
GCCCAGGGGACGCTTGCTGGGAGCAGCCATTTTCAACCCTACTGCCGTAGAGCAGGCGGAGTCCC
TCTTTTCGCG
CGI: 159 range = chr19: 60283718-60285792
CGAGGTGGGGGCGAGGAACCACCCGGACTGGGTCTCCATGGGCGGGGTCGTGGCTTAGGGCAGG
GACAGGTGTAGGGCGAGGGGTGAGTTCGGGGCGTGGACGTGCGTGGGTTCACAGGTGTGAACGG
TAGCCGCACGTGGGCTGGGACTGAGCTGAAAAATCGGCCAGGGGCGAGGCCCGGGTAGGAAGTG
GGTGCGGCGTGGGGAGGCGTGGCCTGACGGTGTGATTGGCAGGCGGAGCTGATCCGAGAGGACA
TCCAGGGGGCTCTGCACAATTACCGCTCGGGCCGCGGGGAGCGCAGGGCGGCGGCGCTCAGGTG
AGAGGGAAGAAGTTGGCAGGGTCTCTGGGAAGCCGGTTTCCCCTCCTTGTGCCTCAGTCTACAAC
ACCAGCCTGGAACAGAACAAGAGTTTTGCATGGAGTCAAGCACACCCTAGTCGAGTCTTGTCTGT
ACCTCCCAGACGAGCTGACCCCTTCTCCAGAACTCTGCTTCTTTTCTCTGTTCCCTGTCCAGGCCCT
CAGTTTCACTCTAGAGAGGTGCTATCCCTCCGTATATCGGATTTCTCCCTACCTCGTTGAACTTGT
TCACTCCCTTTGAGCCTTTTGAGCCTGTGTGTCTCGTTCTGCGCCCTGGATTTCCCCCTCCCTGGAC
CCCTCAGTGGACCCAGTCTTGGTGTCCCCGTCGCCCTCCGCAGGGCCACGCAGGAGGAGTTGCAG
CGCGACCGCTCGCCCGCCGCTGAGACCCCGCCCCTGCAGCGCCGCCCGTCAGTCCGCGCAGTGAT
CAGCACCGTAGAGCGGGGCGCGGGCCGCGGACGACCCCAGGCGAAGCCCATTCCCGAGGCAGAG
GAGGCGCAGAGGCCTGAGCCGGTGGGGACCTCGAGCAACGCTGACTCGGCCTCCCCGGACCTGG
GTCCCCGGGGTCCTGACCTGGCGGTTCTGCAGGCGGAGCGGGAAGTGGTGAGCCGCTAAGGAAG
GGGTCTGGGGGCAGGGCCAGGCGACTGGAGGCGGGGCTAGGGCGTGGAAGGGCGGGGCCGGCT
GCGGGACGGGCGTTCTCTGGTCAGACTTCTGCGTTATGGAAGAGGGGCTGGGTCGGGGGCGGGG
CTTGGTTGTGGGGCGTGGCCAGGTGTTTGGGGCGTGGCCTGATCTGGGGAAGTGTATAGGTGCTC
AGGTTCAGGGCTTCGACGGGGATGGTTTTGGAACTCGGGAGCCCTGAGCGTCCCCCTCCTCTGTC
CCCTAGGACATCCTGAACCACGTGTTCGACGACGTAGAGAGCTTTGTATCGAGGCTGCAGAAGTC
GGCGGAGGCGGCCAGGGTGCTGGAGCACCGGGAACGCGGCCGCAGGAGCCGGCGCCGGGCGGC
TGGGGGTAAGGGGCACCCTGGCGTGGGATCTGAACCCCCTCCCGATCTCTTCCAAATGTCCCCGC
TCTCCCCAGGCTCTCCCCTCCCGCCACTTGCCAGGGCTGACCTCACCGCCATCTTAACCGGGTGTC
CACCTCTCTCTGCCTGCCTGGTGCTGGCCCCGCGTCCCCATCGCCGCGCCCGTCTGCTCCCCTCAG
AGGGCTTGCTGACGCTGCGGGCCAAGCCGCCCTCGGAGGCCGAGTACACCGACGTGCTGCAGAA
GATCAAGTACGCCTTCAGCCTGCTGGTGAGGACGCGCCCGCCCCTGGGCCGGGGCGCGGGCACG
ACGAACCTGTCCCGTCCCCGCACCCACGCCAACCACCTCCCTCCCCACGCCCCAGGCCCGGCTGC
GCGGCAACATCGCCGACCCCTCCTCTCCGGAGCTGTTGCACTTCCTTTTCGGGCCTCTGCAGATGG
TGAGACCCGCCCCAGGCCCTCGGGCCCCCCTGCAGCGGGAGGAATCGGGTTCGACTTGTAGAAG
GTGTGGCGGCACAGCCTGCCCCTCCTGCTCCCCTGACAGATTGTGAACACGTCGGGGGGGCCGGA
GTTCGCGAGCAGTGTGCGGCGGCCGCATCTGACATCGGATGCCGTGGCGCTGCTGCGGGACAAC
GTCACTCCACGTGAAAACGAGCTCTGGACCTCGCTGGGGGACTCGTGGACCCGCCCCGGGTGAG
GGGCG
CGI: 34 range = chr19: 60907042-60907388
CGCTCCCCTGCGTTTCGGCTGCTGGCCCAGCAGAGGGCGCCGGAGAGCGGACGGAGCCGCAGGC
GGGGTTGTGGGAAGACGCTGGGATCCCGTGCGCGCTTGTGCTCCTTCCTTCTGGGCCAGCGCGCA
GACTATCAACCGAGGTATTCAGTCCCTCGGAAACCCAGTTCCTCCGTTTCCAGGCTTCTCGCCCAC
ACGGAGACAGTCGCTCTCACACGTACCTCTCGCGGGCTCTTACTCGTGGTCCCGGTCTCCTCCTGG
TCCCCACCTTCCAGCCCAGCGCGCACACTAGACGGCACGTGCTCTCCACGCTCTTTCTCCAGCGCC
CCTTGCTCACCCGCCCCCCG
CGI: 61 range-chr19: 62043096-62043807
CGCAGCAAGCCTCAGCCGCCCTTATTTAAGATACCGCCCGAGAGGGGGGGGAAAGAAAACCCCT
ACAGGCAGGACAGCCCTTCGCCCCTCCCACCACCACTGCGGCAAACAAGCCCCGCCCCCAGAGG
GTAGCCGGGCACGCCGGCGCCGCGAGGCCGATGGCACCCCGCAGGAAGGGGCATTTTCGCAGCC
CCCAGACCCAGCCCAGGAGGCCCAGGGCTCCCGAGAGCCGCATTCCGCCGTGGCAACAGGGATA
GCATTGGTGTCGACCTTGCTGGACTTGCCGTGGCAGAGCCCCCGGCTGTCTGCCCTAATGCCCCC
GGTTTGCTGCGGTGGCCCCCGGCCAGTCAGAAAGGCGCGGAGACCCTGCAGCAGCCCCTCAGAC
CCCGGACTGCAAGATGGCGGCACCAACGCAGCAGCCCGGGCTGCGCCCACCCCTGCCCCAAAAA
GGCACCAACCATCCACAGCCTGGCCGCCACTGTGCCCACTCTCGGACTGGGCAGCGGGCGGCCA
AGTACCATGTCCGCCAGCCCGCCCTGCCACAAGGGTCGCTGGCTTGCCGCGGGGTCTTGGGCTTG
CGGCAAGACGGCCACTCTGGCCCAGAGCCGTCGCGACACCAAGGTCCCGCGGGCAGGAGGCGCG
CGGGGCGGCCGAAGGCGCACTCACCTCACCTCAGTGCTGCGCAGCCTCGGGCACGAACAGCCGC
CTAGCG
CGI: 94 range = chr19: 63321122-63322053
CGCCCAACGAAGAGGAGGGAGTAGGGGTCCGCGCCCCACCACGAAAGGAGAGTAGAGGGCTCG
CGCCCTGCGGGGAGAGGCGTTAGGGGGTCCGCGCCCGGCGAGGAGATGCGCGTACCAGCCCCGC
ACGCCAAGGGGAGAAGACACGCGGGACGGTCCGCGCCTGGCGGAGAGAGTTCCTCACGCCCGCC
CCGCCGGGAACCACAAGCCAGCGCCCCTAACAAGCCCCTGCCCAGAGCAGGGCTGCCGCGTACC
CTCACAGACAGAAAGAGCGACCACGCAGCCACCGCCCAGCCCCAGCCGCTCTGGAGTCCTTGAC
CCCACCCTCACTAGGCCTTGGCTCCGCGACCGGTGGGCGGGAACGGAGGAAACAGACCCGAGAG
GCCGCGAGAGGACGGAACTCACTTCCCGCCGCCGTAGCGTCCTCGTCAGCTCGCCCTCCGACTCT
CCGCATGGGCGCGCAGAGTTCCCGGATGCGATATTCCGGTGACACCGGACGCTGGGGGCGGGGC
CTAGTGCGACGAGGGCGGGGCCGGGGCCGGCCCGGGGGGGGCCGTGGCCGCAGCTGCCTGGCA
GCCAGAACCTGGGAGCGCTCGCAGTGCTGCCTTTCGGGGTTCTTTGTCCTTCGCTTCTGCTGGTAG
GTGAGGTTTAACAGACGGAAAGACGCCGGGAGCGCACGTGGGCTGCCTTGAGCCGCTGCGGACG
AGCACCTTGTCTCTCAGGTTGACCAGCCGTATATCCGCTTGCGAGCCTGCACCACCCTGGGCGCG
CGTGGTCTCGGCAGAATCTCGTTCACACGGGTCCCACTCGTAGGCTGCTGTGTCTCCTTCCTCTCA
CGGCGTCGGGGACTGGGGCCTGGAGTCACTCCTCTACAGGGGTGTGGAACGCGCCAATGGACGC
TGACACCTTCATGCGGTCGTTTAACGCTCCG
CGI: 35 range = chr20: 9444472-9444893
CGCTTCCCTATCGGGGCTGCACGTAGAGCATCTAACCGCATGTTCCCGGAACCTGGGATGCGCGC
GCGCAAAGGAGCGCCCAAGCGTGCAACCCAGAGTTTGGACGCGCCGCCCTTTACAGCCCCCGCC
CCGGGGCCGGGCGAGCTGAGGGGGCGCGGGGCGTCTGTGTTCCCAGCTGATCACAGAACAGGCC
GATATCGCATTGACCCGGGGAGCTGAGGTGAAGGGCCGCTGTGGCCACAGCCAGTCGGAGCTGC
AAGTGTTCTGGGTGGATCGCGCATATGCACTCAAAATGCTCTTTGTAAAGGTAACTCCGAGCCCA
GCGGGCAGAGGGGCCGCAGGCTCCGCGTGGGTGTGGGAAGTGGCGGCCCCTAGGTTTAAGAACC
CAGACGCTGCGGGGACGATTGAAGCGCACCTCCCCG
CGI: 671 range = chr20: 21433933-21444714
CGCGGAGACATCTCGAGTCCCGCAGGTGTCTGAGCTGCGCTGCGCGCCCCAGCCGGGAAGCCTTT
TCTTATCCAACCGCGCCGCCCCGCGGAAAGCCTGGGTACTGCAGACGCCCTCACCGGCCCACCGA
CAGCTGGTTTTGAAGCGGGGGCTTAGAAAAGTAAAGGGGACAGTGTTTGGAGCCACGATGCCTTT
GATGTGAGGCGGTTCCACCCAACAATCAACGAATCAAGCTGGAACTGCAGGCCCTACTTTTGTTG
GGGGGAGGACCTTAAGGAATGTCACCCCCTTGTCCCCTACCCTCTGCCCCACGCCCAGCCTCTCA
GAAGGCACCAGGCGTGAGTAGAGTGCGAGGCGTCCACACCCTCGTGTCCCTTCCATTCCCGGACA
CCTTTGCACCCTAGCCGAGGCCTCAGCACGGAGTGAGGGTTGCCTGGGGTGCCCTGCTGTGTGTG
GGGCGTTCTTGCCTCCCGGGCCCTGACCCCACGGTCTGGGTCGGCGAGGCGTCTCTCCCTGGCGT
CTGGACTGTGCGCCTGCCCCGCTTCCAGGCGACGAACGGCAGGAGCCGGGGGTCCGGCGGGACC
CGGATCCAGGGCGGGCGGAAGGCGAGGGTCTCTCCTTCCCGGAACCCCCGCCCCGGAAACCTGC
AACCAAACCGGGTCCCTCCTCCGCATCGCTCCCGCGCTCCCGGGCCGCCTCCCCACGCGGGCACC
CACTCTCCGGGCCCTCGCAGCACCGCCCTCTCCGCGTCCACCCGCGCGGCCGACGCCGCCAGGGG
CCCTGAGATCGCGGAGACAGCGCGCCCTGGCGCCTGGTGGGGCCCTCCCAGTGGCCAGAGCGCT
CCTGGACCAGTCGGCTGTGGTCTGTGCCCAAACACGATGGAAACCGGGGCTGGGACGGGCGGTG
GTACGCACGTGGAGGAGAAGAGGCATGGCGGGAAAACCACAGCCGAACACAGGTCGACTCGCA
CCTCCCAGGCACCCGCCGCCGTCTGACTCGCGGCTCTCCCCAAGGTTCCGACTCGCCGCCACCTCT
TGGCACCACTCACCGGGCTCCGACCCGGGCCGCGAGTCCGCTGATGCGCAGCTGCCTCCGGCGGC
TCCCGGGCTCTGCCCGGACCCGCGCGCAGAGACGCTCGGGAACGCTGGGCTCGGGCGCCTCCTGG
CGGCGGCAGCACGCGGCACACGCTCGGTTCTCCGGCCCGAGGGGGCGCGTGGAGCGTACGCGGG
ATCCGCGCGCGGGCCTGGGCTGCTGGCCCACATTTCCCCGCTTGCTCGGATGGCGGCGCTGAGGC
CTATTCGCCTCTTTATTAATAACGATGCCTCGGGAGGGAAAAAATAGAGCCTTTAAAAATCAGAG
TGCGAGAGAGGAGTGTGTGTGTGTGTGTGTGTGTGTGTGTGTGTGTGTGTCTCGGGGCGTTGGTG
AAAGCAATAAAACTCAGATCGCTTCCTTGGCGAGATAGCGGCGGCTATAATGCGCCGGGGGGAT
CAGGGGTTTATTTGCAGATCCTGGATAAAAGATGATATATCGACACGGTTTAGAGTGTCACTGCG
CTTGGCCTCTCAGGGAGCCACAAGTACATATTTCCTGAAATACCGCCCAGCACAGTATCTAATCC
GCCCGCCACTTAACACCGGGAGGGGAACTTCAGCAGCGCACAAGCGCGCTCCCGGTTTCCTGGTT
CTGGGTGGGGAGAGCCCAAATGAGAAGGATCTGGTATGCCTAGACGCCCCCTCCCCTGGCCTTTG
TCTTCAGTCCAAGTAATCACTTAGCGATGGGCCTTAATAAATACGTCTGAGAACTGTCCTTATGCT
CGGTAAATATTGGATTAGCTTTTCAATGACCAGGCTCTTGAAAAGGCTCGAGAAATACACACCAG
GTCTCAACTAAGCCCAACAGTCAATAGGTAAGGAAATAAGTTATTTCCCCATCTGAGGTGGGGAA
TGTTGGGAAGGGGGACTTCCACTCTTACCAAATTATCTGATTCTACTAAATATGACCGTTGTACTA
AATAGGAAAGGGGGCGCATCCAAGTTTCCCATTAGTCGGAATTTCCACCAGCTCAGTTCTGCTCA
TTAAGGGCCGAGTGACTTTGTGGGGAACATATCTGGGTTCCAGTTTTTATTTCCACAGAGCTCAGC
ATAAACTATTTTTGTTTAGTACATATGGTTTATTTGGGGACTGCTTATGGGAGTTTTGACCTGGCA
ATTGCAGGGGAGAAGGCATGACCCAAATTATTTCCGACTGCCTCGCGCCAATTTGGACCGCTTAG
AGGAGGCAAAAAATGGGCAAATTCCTTGGAAGCCCTGCTGTGTCTCCCTGGTACTGAATTTTGTA
AGCACGCGATACAGGAGACATTGAAAATTAAAGAGGAAGTATCTCCCTCCTCCAACATGGGGAG
AGACGCTGTTGGAAGAAGTTTCTGAAGTGGTTTCCCATCGCACCAAGAGGCCAGTGGGATGGATC
CCCGCGCATAATTCAAAGGAACGCGCGCGTCTTTAGCTCTTCGGGGTTGGTAACCAAGTGGTCCG
GCCGGGCGCAAGTCGCCTTGGAACCGGCTGCAGCCCAAGTCTAGCTGGGCCCGGGACTGAGCGG
CACCCTTGGCCTCTCAGAACCTTTCTCGCGGTGAGTGGAAGGGCGGCTCCGCGCGCGGGCGCTTC
GGGGCCGGTCGGTTGCGAAATCAGGGGGTGGATTTTCATCTACCCAGGAGCCGCTAAGCCCGTAG
GGGACCGAGCATCACCCAGGCCCTGGGTACCCTGAGTTGCCTCCTGAGCCCCAGGACGGTCCGAT
GGTGCAGAGCTCCCAGGCAGAAGTTAGTTTCCTGATACACACACAGCAGTGAGCAAAGTGCGCA
CAAGTGAGCCAAACCCTGAAGACGCAGGTTCGCGGACTCCCATCCCAGCGTTAGAGTCAAGGAG
ACAGAAGAGAGGAAGGAGAGAAGTGAGGAAGGGAGAGAGATTATGAGAAAAAGAGATTATGA
AAGTAGAGAAAGAGATTATGAGAGCGAGATTATGAGAAGAGAGAAAAAGAGAGATTTTGAGAG
AGAGAGACAGAATGAGAGAGATCCAACTTTCAGTGTCACGGGGCCTAGGCGGGGCAAGCGCCCT
CCGAAGCCATCAAAGACAGGGGTGGCTTTTTCTCCCCTCCTCCAGCCTCCTCCCCAATAGATATA
GCACGAATTTTTAATGCGTCTTCCTTTCACAGACAACAATACAGGGTTTGAAAGCCGGACACAAT
TTAGGTTTTGTGCGGGTCCCTCCTCGCGCTCCCTCGGCCCAGCTGGCCATATGGAATCCAGCATGG
AGCCCGTGGATGACAAAGGGACCCAGTGACAGGCCCAACAAAACCCCAAGGAGACTTCTGCCTG
ACTCCGGGATTCACTGCCGGCGATAAGCGCGCTGAATGGTGCGGGGGGGGCGGGGGGTGGGGG
GCGGGCCCCTTCTCCTTCTTCCCCTCTCTCTGTCCCCGCGATCCTTTCTCTGTCCCTTTTTAATATGC
ACGCCTCTAACATCTCCGGCTCCCAGCTTTCCCATTGTGCGGTTTTGTACCGCCCGCTGTAGATTG
GGCGAAGATAGACTGCTGAAGCGGACCCCGCGCCGGGTGCGCTCCCAGGGCGCTGGGGTTAGGG
CCACACAGGGCTCCGCAGGCGCTTTTATTGACTCCATGGGGACCCGAGGACAGAGAGAGGCGGG
CGCCGGGTCCACCTCCCGGGCTCCCGGCCGGCCACAGGAGCGCACACCCAGATTGCAGGCCCGC
GGTCCTGGCCACAGCTCCAAATTTGTCCTCAATGAGCAAGTGGTGGAAGCCTGCCAGGGTGAGGA
CCTTCCACGCTTCCCCCTTCCTCTCTGGAAACCTGAGGGTCTCTTGTGATCATTCTCACCCTGGAA
GCCCTGGGGCCTGGAAAAGTCAAAATGGCCTCAGGCCTTCTGACAGCTCCGCTCCAAGAGACTTG
AAACCCTCACCTTTCCTCCCAAGAGGATTTTGGGATTAGGGGAAGTTGCAAGTGGATGTTGAGTG
GTTAGTACCAGGGCAGGTTGGGCGTTCAATTATTGCACAACAAACTATTTGGGAGAAATTCGTTG
GCCTTTAAAAACAGTAAGACATAAGCCCCCAAACATTGCCAAGCTAGTGTGGAGAACAAAGGGA
GACCTTTTAGCATTTAGAAAAACTGCCCTTTCCCCGAGGATGAGCCCGGAGATGGGAAAGGACCT
TTACTGGGGTATTAGAAAGACTTCCGAGCCCTCAATTATCCAGGGGCGGTGTGGAACGCGGGAAT
AAAGTGTCATCGCGTCCCTGGGCTGCTGCGCAGAGTGCTATCGACGCACAGATTTGGTCGCACGG
ATAATCATGTTTGCTTTAGATATTGAATTAAACCTGATCATCAACTACAGGGAGCGAGAAAGTCA
GCTGGGACCGCTCCTTTTGCCCGGCCTCCTCACTGCCACCCAACCCCCCAGCCTTGATGGTGCAGC
TTCGGAGAGAACAGAGATACAATTTCCAAATGCGCCGACGTTCAGTCCCTCAGCGTCTGCAGCAC
CCGCTGGGCTCTGAGCTAGAGTCCCAAGTATTTTTAGAAACCCGCGAGATAGAGGAGAAAGGCT
CATCCTCCAGTTGGCCAGGCTTCCTAAGACGCTCCCCCGCGTGGCCAAGTGTGCGCCAGCATCTG
CCAAGGAGATCTTCCTCCAGCAACAAGGGTGGGACGGAATCCGTTCCCCGCCGGAAAGATGGAT
GCGCCCTTCCCGGGCGTGCCCGGGGCTGTGTGCCCACTTCCCATCCGGGGAGCTCACCCCACCCC
CAAACCTCTACTTCCTAGCAATCCACCCAGTTCCACGCTCAAGGGCTGACCTGGGGGGACCGCAC
ACCCTGGTTCGGACCGCCGAACGCTGGAGGGAAGGAGGCCCGGGATCCAGTCCCCGGAGAGAGG
CCTGGAAGCCCGGAAAGGATGGGAGCGTAAGGGAAGGGAGAGGCATGGTTTGCTGGGATTGCCA
AAGATTCTCGCTCCCTGGCTTCCTGGGGCCAAAGATCCTAGGGAGGGCTGCAGTATCCCCTCCCT
CCCCCGAGCCCAGAACCCCGCGTACTTCCCCCAAGCCCCCTAAGCCAAAGCCTCGCGCGCCCTGA
CCGTTCCCACCCCCAGCCTTAGCGGTCTCCTAGGTCAGCACCAGCCCGCAGATTTTGAAAAGGAG
TGGGGAGTGGGGAGAAGGCAGCCAGTCTTAACCTCAGACCCCCACCCACCCACTTTCTTAGCGTG
AGGTAAGTCAAATGTAACAAAGTGAAAGTCATCGAGTGTAGTGGCTTCAACAGCTTCTCAGGCTG
AAGGAGCCACTCATAGATTTGGGCCAGCGAGGGGGGTTTGCTCTCCTCGCCTTTGCGGCTCCCGA
AGTCACTTTTCCTCCCGTAGCCGGACGCTGCCGCCCGGGGTTCAGACAGCGCGCGAGACCAGGGG
AGATGCGGGGGAGGGTGGGCGGCCCTGGGCTTCCGGCCGACCGCTCGCAGGCCGGAGGCCGAGC
GTTCTCCTCCCCGCCCCAATCCTAGGCGCGGGGTGCAGCCTGTGGTCGGAGGGGCCTGCACCGGG
AGCCCCGCCCGCCCCAGGGCCCCGGTACCCCCGGCCTCCCCGCCCGAGAGGAACCGCCTGTGGG
AGCCCCGCGGGCGGCCGGGCCCGGCTCCCAGGCCCAGGCCTCCGGCCCGGCGCAGTAGGACAAA
CGGGAGGGCAGGGATTTTTTCTTTTTTTCTTTTTTAAACACAGGATTTTTATTAAAATTCTTATTTA
AAAAATCGAAAGCTTTCTGCGCCCGGCGCTGTTGGGGAACCGCGCCGAATAGCTGAGCTCCAAGT
TCGGGGCTGAAGGCCCGGAGGACAGGGCAGAGGGCTCCCTGCCTACAGGGTTTTCTTTTCCATAT
TTGAGAAATGTTTGCACAATAAAATAAAAAGAGAAAGGAAGAAAGCAGGGGAAAACGCAAAAA
CAAAAACAAAACAAAAAACAAACCCAAACAAGCCACAAAGAAAGGAGTTGGACCCAGACGGGA
AACAGGCCGGTCCTGAAGGTCATTTTGGCAACAATCACCACCGATATTTACAACGAAAAGCGAA
ATCTGCCACCAGTTGTCAGAACGTTTACATGGCCATAAATATTTAGCATTTTCTTATTTTTTTTTAA
AAAAAGGAAGAAATTCTCTGTATTTTTAAAGTGTTCTCCACTTGCTTTAGAAGACGGCTGACAAT
ATCGCTACTCACACAAACATATTTTACAAAATGCACGAAAGCAGGGGTGGGGGGTCGGTCTTTTT
CTCGTTTTCAAGTGACGACATTAACGCTGGGACGGTTTGGTCCCCCCGGGGAGAGGCAAAGAAGC
GAAGCTGCGCAAACATTCTGTAAACACGGCGTAGAGTTCAGCCCTCTCCCAAGGTTCAGAAGGA
GAGGCATGGGCAGAGCTGGGTGGGTGGAATCTGCCACTCCAAGGAGACGCAGGCAACCTCCCGG
CGCCTCCCTAGTGGAGCCGAGAGTCAACTCGACTCCATAATAATAATTATAATAATAATAATAAC
CACCATAAGGACCGAGGCCTCCTCGCCGCCACCGCCGCCGGGGTGGGGCCTGGGCCTGGGGCCG
CGAGTCTCGTTGGGGCGGCGCTCACCAAGTCCACTGCTGGGCCTGGACCAGGGGGTGTGCTGTCG
GGTACTGGGGGGTGCTGGCCGAGCTGTACTGGGCGTTGTACTGCATGTGCTGCAGCGACTGCGCG
CTGTAGGCAGAAAAGGGAATGCCCGCCTGGAAGGTGGCGGCTGCCAGGTCCTGGGCTTTGAGCG
CGTGACATGGTTTGCCGTCCCTGACCAAGACGGGCACGGCCACCCGGCGCGGCGAGGGCAGGGG
CGTCACCTCCATACCTTTCTCGGCCCGGGCGCGCTTCATCTTGTAGCGGTGGTTCTGGAACCAGAT
CTTGACCTGCGTGGGCGTGAGGCGGATGAGGCTGGCCAGGTGTTCGCGCTCGGGCGCCGACAGG
TACCGCTGCTGCCGAAAGCGCCGCTCCAGCTCGTAGGTCTGCGCCTTGGAGAAAAGCACTCGCCG
CTTTCGCTTCTTGCCGGCGTCCCCCCCGCCGCCCGGGGTCTCCTTGTCATTGTCCGGTGACTCGTC
GGCCGAGGGCTCCGGGGACTTGGAGCTTGAGTCCTGAGGGGGCGCCCCGGCAGCCAGACCGTGC
ACTGGGGGGAGGGGGAGAGAGAAGCGCGTCAGGCGTCTGGAGGCCGCGCGCAGCCTGCACCAG
ACTCGGAGCACCCTGGATCCTCCGTGCGACCCTGGACCTCCGCGCCTGGCAGCCCAGCCCCGCTG
CCTTTGCCATGACCCCTGCCTTCCTCTACGCCTGACGAAGACGTCGGGACCTCCCAGGGTAAAGA
GGGAAATCCGTAGGAGTGGGGGCTAGGGGCTCGCCTTTCAAGGGGCTCACATAGTGTGGGTGAG
AATTTGGGGGGGGAAGTGGCACTGAAGATCATCGCGGGTTAAGGGCTCCGGCGCCCTTAAGCGG
TTTCCTCTCCGGCGAGACGTGGGCAGATTTTTAAAAGAAATACCCCACAATCCCAGCATTGATCG
GCTCGGCCATTTAAGGCAGGTTTTTGGGGGCTTTCGTTTCTGCTTTCGGGCCTTCTAGACACCCCC
TGCACTGCACAAAGCGTCCTCTATGATCGCGCAGGGGGCAGGGTGAGCTATTTATACCCGGGCCA
CCCGAAGCCTCCCCACCCTCCACCCGTACCCGGACGAGGCTCGCTGGCTACCTCCAGGGCGCCAG
GCCAGTCCCTCCCAGCGGCCGGAGTCCGGGGGCTGCGGCTGGAGCCATCGGTCCGGGTTGACATC
ATATACCAGCCCCTGGGAGGTATAGCCCTTCTCCCTCTTTCTCCTTAACTTCTCGCATTGAAAAAA
ATTTTGGAGACCAAGTTCTTTCCCGGAACTAAGGAGGCTTGAAGAGGAGGGCAGAGGACATCCT
ATACAGGTGTTAAAAATCTTTCTACGGATCCGAGTGAGGGGGTCCGGGCTTACATGGCCCCTTCC
CCTTTCACTCCCAGCGTCCAACCCGGGCTGCGGCTGCAGGAATGGAGGGGACCACGGCCTCGGCA
GCCAAGTTTTGCTACTTACGGGAGTACTGAAGGCCCTCGGTGCTGGCCAGCCAGCGCGTGTACGG
GTTGTCGCTGCTGTCGTAGAAGGGGTTCTTCAGGGGCAGGCTCTGCACCGCGTCCAGGGCGCCCT
GCCCCAGCGGCCCGGCCCTCTTGGCTGGCTCGGGCCCCTCGTTCTCTTCCTCCGGACCTTCGGCCA
CAGAGCCCTCCTCATCGTTGGTGTCCGGCAGGTCTAAGATGTCCTTGACCGAAAACCCCGTCTTTG
TGTTGGTCAGCGACATGGTTCGAGACCCCAAAATTTATGTCGCAAAGTTGTAGCTTCACTTGGTC
AATTCGTGGCGCTCCCCTGCCCCGGCGGGCGGGGGAGGGGGGAGTTGGGGGGAGGGACTGGGGG
AGGGGAGGGGGGAATTGGCTTTAATTATTGGGATAATTATTATTTTTAAAAAGAGAAAGAAACTG
GGGATGGGGAGGAAAAAAATGAAGCCCAACCCAGTGCCTCTCTCTGTCTTCTTTGAAAGCACGCG
GAAATGGACGCAGGAAGCCGGGCGGCCTGCGCGCCGAGCGCCGCGGGCCCCGGCCTTAGTTTCT
AACTCCAGGAGGGGTGCCAAGGCGGCGTCAGCTCGGGCTCCGGCGGCCGCTCGGCGCGCGGTGG
CGGCTGGTGGCGAGGAAAAAATGGGCCATTGCCCGAGCGATCAGTCCATATAAGGCTGGGCTCC
ACTCACGAACCTGGGGAGGCGGGGAGAGGGGGAGAAAGAGAGGGAGGGAGGGAAAGAAAGAG
GGAGGGAGGGGGAGCGAGAGAGGGAGCGGGAGAAGGGTGGAAAAAAGGGGGAAGAGACATTA
AAAACGCAAAGGTTGGCCACGTGTGGGCGGGTCTTGGGAGTCAAGTGGATGAAGACAGTATTTG
CAGATGTGAAATTGTGGGTTTTGGGGAGCTCCGCGCTCCCAGCCAACGGCCCTCTAGAGCAAGAT
GAGAGGTGTAACGTGTCAATTAATTGCAAAGACGGGGCGAGCCTTTTTTTTTTTAAACCCAGTAT
TTACATACAAAGGGCCACCGCGTCGCTCGCGAGTCCACACACTTGAAAGGGCCGTTTTAACAAAT
TGCATCTTAAAAAAGGTGGGGGTGGGGAGGAGGAGGGGAGAAAACAAAACAGAAACCAGGAGG
AGGGAAAAAATCCTCTTTAACATTCACCGGTTCCTACCTCCCCGCCCCCGCGCGCCCACCCCCGG
CAAGCCGGAAAATTGGCGATTTGTGGCGCCTTTGGAAAAGGGGGAGGGGGCGAAGGCCGAGCTG
CGGAAATCTCTCCTTTCCATCGTTGGTGGTTGCCTAAACAAGATCCGGTTCAAATGGCAAGAGCC
CAACTTCTGTAAGCCTCCTAGGTCAATATTTTGGTTGAGGCTTAAGGATGAGTGCTAGAAATGAC
AAGGCAAGTAATTGATTCCAGTTAACCGCGAGAGAGCCCAAAACCCAGGAGAGAGCTCCGCTCC
GGGCCGGCCAGAGCCACCACCAAGCGACCCGCCCCCTTCCAGCCGCCCTTTATTTGCTAAAGACT
CATTTATTTCCTTCACTCCGCCTCCCCCCACCTCCCACCCCCAACCCCCCGCCTCCACCTCTCTGCG
GCCAAGGAGGACGCGCAGTTCACTTTCTGCTTTGCGGGCGGTCCCGGGGAGGGCGGGGTAAGAG
TGAGGAGCTCCAACTTCGTCTGGGGTCGGACCCTGGCCCTGCTGCCTCTGGGCTCTGCGGGCCTCT
TTCCCCGGGTGGAGGAAAGGGCAGACGGCCCTGCTCTTTCCTGCCCTCTATTATCCCTCCCTCTCC
GGCCCCCTCCCTCTCCAGCCTCTGGCTAGAGGTGGAGCAGCGGGAAGGCGGCGGGGCGGGATTG
CGGGGGTGGGGGTGGGGGTTGTGTCTCCAGCCTGGGTGAAGACCCAGAAAGCTGCGGGGGAGGG
GGGTCGCTGGGTGGCCCGACGATGCCTCGTTTTCTCCCCAGTTTTCTTCTCCCACGCGAGTTCTTT
AGTGTGGTCTCTTTTTGGACGCCTTGGGTGCGTTCGGAGTACGCGCTCCACGTTTCCAGGTTAAGT
CAGGTTAGTGTGAAGTTTCCAGCCACTCTCGGCTCTGGGAACTGAACTTTCCCGGGACTTGCACTT
CCTCCTCGGCGCTGGGGTTGGAAAAGACCACGCCGCATTTGCCACCCCTTCTTCTAGTTAAAACTT
TTTTGGGGGGGGGGTGCAGGGGTGTAGATTGGCTACCTGATGCTCGTTCGCCCCAGAGGAGTGTC
AGGAAGGAGCTTTGTCTGGGGGCGGGGAGGGGCACAGAAGGCCACCCCTGCGGGGTCCAGGTCT
AGTCTTCCCACAACCTGGGCTCCTCCGCGCGGGCCAAGCTGAGCGCGCGGGCACCGGGTCCTCCT
CGCGCGCAGCTGCAGCGGACGCCGCCGCGCCCCAGCTGCCCCCGGGTTGCCAGCGCTGGGTGAG
GGCACCTCTCCCCGAGCCGCTCGGCGGCTTTCTGCAGCCCTAGCTGCCTCGCCCCCAGTATGTGAC
GTGGGTGACAATGGCCCAGGTTAGAGCAAGCCCCTCGTCGGCGCGTCCTGGGTGGTCGGACCCG
GGCAAACACAAATACAAACCGATTGCTAAGCTGCGGACAATGAGCGAAATGTAGACAAATGTCC
CGCTCCCGTTGGAAGCCTTTGTCCCGGCTCG
CGI: 61 range = chr20: 23294035-23294650
CGCCACGGCGTGGCCACCGAGGTGGTGTACCGCTGCGACACCTGCGGCCAGACCTTCGCCAACCG
CTGCAACCTGAAGAGCCACCAGCGCCACGTGCACAGCAGCGAGCGCCATTTCCCATGCGAGCTGT
GCGGGAAGAAGTTCAAGCGCAAGAAGGACGTGAAGCGGCACGTGCTGCAGGTGCATGAGGGCG
GCGGCGAGCGGCACCGCTGCGGCCAGTGCGGCAAGGGCCTGAGTTCCAAGACAGCGCTGCGGCT
GCACGAGCGCACACACACGGGAGACCGGCCCTACGGCTGCACCGAGTGCGGCGCCAGGTTCTCG
CAGCCGTCCGCGCTCAAGACGCACATGAGGTACGCGGGGAGCCGTCCGGAGGGGTCCCTGCGCA
CTGGAAGGGTGTGTCAAGCACTGAGGTCGTCCTGGGATTTGATGTCTCACTGCTTAATTTCTCCGC
TCCAAACCTGGGGGCTTATTTCGAGACAGCGTTTAGTTCTCTGTCAAGGTACAGGGGCTGCGAGT
GATGGCTCTGCTCCCTTGTTCTGAGACCCCTGGGGCGCCCCTGCTGGCCGGGACGTCCCTCTGGTG
GGACCGCGACAGCGGCCGCGGGCTCGCCCCACCG
CGI: 118 range = chr20: 25011839-25013525
CGCGCTTTGAACCCAACCAGCCCTCATCGCTCGGAGCCCTGCGTCCCCCGCTTCCCAATCCCAGA
ACCCCGGAAGCAGATCTCCTCAGTCCCACTCCCTGAGGGTCCTACAGTCCGAGGTCCCGCAGCGA
GGGAGGAGCAAACGCCTGAGCTCCGCGGGAACCCGGCGCGGCTGCCTGCGCGCACGCAGGGACC
GGAGCCCGAGCCTGGGCCCTGCAAAGTGGGTCTCCCACTCGCTGCGCGTTCGAGTGGGGCCTCCG
CTAAACATGCAGATTAAACAAGGGGCTTCCGATAAGGATAATTACAGGAGGCTGCGCATTGACTT
TCCGTTACTAAAGGAAGACCCAAATCTCCCCCTTGCAAGCTGTTCCCGGGATTACCAAAACAATC
ACTTTCTTTTGAGATCATTGTCTTTTGAACCACTCCAGCTCGGGGCAGAACGCTTTAGACGCTGGC
GTTGTTTTCCGCTCCGCGAGGAATTTTCCCCCAGATTTATCTCTGGGTTTAGGGAAGGCGTTGGGC
ATTCGGGACTGCGTAGTCGTTTGTAAATCCGAATTTGGGGTAGCAGTTAAGTGAGATACCTACGC
TTCCACACAGTTTGTAGCCCGAGACCAGTCAGGCAGGCAGGGTAGTCGATTCTAAGGTGGCCCGA
CCGGGGTCGCCCGCGAATGGAAAGCCTAAGGGGCGAGGGCGGACGCGGGCGCCGTGCCTGCAGC
CCCGGGTGCCGACCAAGTGGCCTCACCTGGGACAGGTCTTGGGGTCTCCCTCCCCTAGGTCTCCC
TGGAAGGTAGGCGATCACGGGAGTGACAGACCCCGAGTAGATAGCCAGTGATCCAAAGACACCA
AAAGGCCAGGCGGTAGGAGCCCAGCCTCAGACACTCTCTATAGTGACCGCGGCGCCTCGGGAAG
GATCGTCTTAAACTCATTTTGCAGGTGAGAGCAACTGGGACTTATTAACCCAGTCACATCGGACA
CAGAGACGTGGCGAGGGCGACACAGAGCAGGAGACTCAATCAAGGAGCCCGTATTGGAGCTCTA
AGGGGACTCAGAGATGCAGAAGGAACGAACTTCTAAGGGGTCTTGCAATTCCTGGACTGGGGCG
TTCCTAATGCATCCCGGGACCCCAATGCCAGGGAGGGGCCTGCAGGACCCCAGCGGTGGGCGAG
TTGTGTCCTGGGTCACCTTGTGTTTCGCAGCGTGGCGGTGGCAGGAGCCCAGCGCGGGAGGACAT
TTTCATAGCCTCCTACAGTGAGAAACGCCCCCCACCCGACGCTGCGCTCATCTGTGTCCCCGCTGT
TGCCGGGGCTCTGGTATCCACTTGCGCGCCCTATGTGGTGGGGATCCACCCAGAGCCCAGCGTCA
AGTTATACGGGCGCTTCACTCAGCGTCAGCCAAGACCAGGGAAGCGCTTCTTGCCGTTTAGGAGA
CGTCTGCAAGAGATAAAAAGCTAGCCCACGATCCACCCACAATCCTCGTGTCCCCGGGGTGCCCT
CGCAGTTGCCAAACCTACGGGCCGCGTTTAGGGGAAGCCTCCGCGTCCTGGCGGCCAAAAGAAT
GGGCTCCTTCCAGCTTCCCCCTACCGGATACCACCTGCAAATCTATTGCCAGAGGCGCAGCTCCC
GCGAACGTTCCCGCCCCGGGCGGCCCCCAGGAGTCGTTTTGGGTCCCTAAATCCGTTGAGGGTTC
CG
CGI: 380 range = chr20: 36785545-36790786
CGGCCGTCGGAGCAGGGCCCCAGCGCACCGCGAGCCTCTGCTTCCTCCGCCTTGCAGCGCCGCGA
GCCCTTCTTTCCTCCGCGCCTGGCCCCGTCTGCGCCCCTCTCGGCGTGGGCATTCGCAGACCCGCT
AGGAGGCTGACGAGAGGTGTCCTCATAGAGTCCTGCCTTCTTCCAGACCCCCAAACGAAAAGAA
AGAGAAAAGCCAACCTTTCGCCTCCATTCTGCACGCATTTGGAGAGCGGCTGGTGGCGAGCGATC
CGCACAATCGCTTCCCTCGCGGCGTCCCTGGACGGGCGGAGAGAACCCGGGTGACTGAGCCTGA
GGCTGAGCTTCCTTGGGAGCGATCCATGCCAGCCACTTCTACCTTCAAGGGGGACTCGCGGCCGG
GTTTACGCGCGAAGGATGGGTGCCAGGGAAACTGACTTATAGACGACCCGCTCCGCGAGAGACT
GACAACTCAGTCCTGCGGGCTTTCGGGGCGCCCAGACCAGTCCTTAGCCAGAGGATCTGGGGCTG
CACAGCTCCCGGACCCTGGAGGGGGTGGTCCGGGGGTGGGAGACTGCGAGCATCAGCCCACGCC
GGCCCCCTCCCACCCTGCGGCGGCGAGGTGGGAAGGGCCAGGATGCGAGCTTAGCACCTCCCCT
AGAAATGCAGCACTTCGCCCCCCCACCTCCCCTGCCCTCGGCGCTCCCTCTTTCACGCGCTCCCTC
CCGCCTCCTTCCCAAGGGCGCCCTTCTTCTGCCCCCAGCTCACGTCTGAATCCCTCGGCGCCCCCT
TTCTCTTCTCCTAGCCCCTTCCTCACGTCCCCTGCCTCCGGGTATTTCCTCTCTCCAATCCCCCACC
CCCGCACCGCCTGATTCCGAGGGGGGGGAGCGCATTGGGCTGCGCACGGGTGGGGGCGCCGCGC
CAGCTTCGCGTAGCTGCTCTGACGCCGCTGCCGCCGCCGCCGCCGCCGCCGCCCTCCGCAGCCCA
GCTCGCGCCCCGCGGCAGCTCCGCAGTGCACTAGCCACCACCGCCGCCGCCGCCGCTCCGCCAGA
CCTGCTGCCAGCTTGCCCGGTCCAGCCCTGAGAGAGCCTCGAACGCCAGCTGCGAGGGTCATGAG
CCAGAGAGCCCCGGGGCGCCGCGCGGAGAGCAAGCGGAGATAGCGACTTTGCGCCCCCCAGCCC
TCGCCTTCTTGCATCGCGTTCCCCGCATCCTCGGGTCCTTCTGTCCTTTCCGCTGTCCCCACCGCCG
CCATGGCCACCTTGCTCCGCAGCAAGCTGTCCAACGTGGCCACGTCCGTGTCCAACAAGTCCCAG
GCCAAGATGAGCGGCATGTTCGCCAGGATGGGTTTTCAGGCGGCCACGGATGAGGAGGCGGTGG
GCTTCGCGCATTGCGACGACCTCGACTTTGAGCACCGCCAGGGCCTGCAGATGGACATCCTGAAA
GCCGAGGGAGAGCCCTGCGGGGACGAGGGCGCTGAAGCGCCCGTCGAGGGAGACATCCATTATC
AGCGAGGCAGCGGAGCTCCTCTGCCGCCCTCCGGCTCCAAGGACCAGGTGGGAGGTGGTGGCGA
ATTCGGGGGCCACGACAAGCCCAAAATCACGGCGTGGGAGGCAGGCTGGAACGTGACCAACGCC
ATCCAGGTAAGCGCGGGATTCCCAGTTCTGCCTGTCCTCCCCCCTCCCAGCTCAGCGTGCCGGGCT
CTGCCCCCGACAGTCGCCCGGTGATCTCGGCCTGGAGACCCCCTCCTGTACCCAGGAATCTCCCTT
TCTCCATCCCTCCCAGCCCTGCGCGGGGACCTACGCCCCCAGGCGGTGTTTTCCGCCCTAACCCAC
GCTCCCTCCCAACGGCACCAGCTGCAAGACCGCTAGGCTGAAGTTCGGTCTGAGACACCTGTCCG
GAGACACTGCAAAAGTGAAGGAAATGGGGGGAGGGAGCAGGAAGCGATGAGAAAGAAAGAAA
ATCAGGATTGGAGGGCACGGTTTGGTCTTGGACTCTGGAACGGATTCACAGCTGCATTTTTGGGA
GGAAAGAAGAAGGGGAAATCGCTGAGGTCGGAGTCTCCTCCCCCCGCACACACGCATAGACACG
CACACGTATATAGGCAGGCACACCACATGCCCACAGCCCCCCTTCCCACGAACAAAGGCGCAGG
CACAGGCGCACACGACATGTATCTATATATAGACCACAGTACTATCCCCAAATGCACCTGCGTAC
ACCCCTACAGCTCCACAAACAAAAGCATATACCTGCACATACCAAACACACGAATATTCGTTTGA
ACATATGCACACACACATGCACAGATGCATGCAGACAGGAACCAGCCTGCTCCCTTGGGTGCGG
CCCTGTTGGAGACATACAGGTATACACACACGAAAGCACACACATGCTCCCACACAAACACATTT
AGTCCGCAATAATCCAGCATTACCTGTGCACAGACTCGCCCCTCTCGCCCGAAATCCCGGCTGCA
CCGCAAAGAGGCGGCCAGCTCCTCCCCGTGTGACCCTGGCGAAGCCCTCTCCAGAGGCTAAGAAT
GGCCCCTGCTCTCTAGGCCCTCTCCCCTTATTCCTAACCCCCCACTCCCCACCAGGCGGCGCAGGA
CTCCTACATAGGGAGCAGGTTTGGAGTCTTGGCAACGACCTCCGAGCTAGCAGGGGCTGGCAGG
CCGCAGCTTTCTGGGCCGAAGGAGACTTCAGCTCTAGGCAACCCCGGTCCCTTACAGCTTGGCCC
CGCGGCGTCCCTGGAGCGCACTATCACTGGGGCCACGGAAGGCAGGTTTTCTGGGAGCAGAGGC
CTCCCAGGGGTTGTTCCATGTATCGGGGTAAGCAGGACTCCACCGGGCCCCGAACACCAGATGGC
CCGACCCAAGGCGCTTTCCGCTCCGGGGCCCGGCGAGACGGCCTTGCTGGATCACGGACCCGGG
GCTGCTCCCGCCTTCGCTGTGCCCCGCGACTTCCCGGGGCGTCTTTCAAGGCTCCGTTTGATAGGC
CCCAAGGGAGGCCAGGGCAGGAGGCTGGAGGCTCGTAGGCCTCCGGGACCCTGGATTTCGTTAG
CTCTTAGTCCCCGTGTGGATTCTAAACCCTACAGCCCTGTCCGCCTGACCCTGAAGCTTCTGGTTC
TAGACTCTTGCTCGGTTCGGCTTTCTGGCCTCCCTGGCGCCTTCCCGCCAAGCTCCTCCTCCTGCTT
GCCTCCCCAGCCTCCACTTAGTCCCCTTCCTTAGGTCTCGCCCTCGCCTCACTCTAACTCCAGCCTT
GTCCTAAGACTCTCAATTTCCAGCCCTGCTTCCTCCTCCCCGCGCGCTGCTCCCCAAGCGTCTTCA
CATTGTCTCTGTTCTTGCCTCCCAGGGGCTTTGCCCCACCTTCAGCCCCTCTCCCTCTCTACCCTAT
TTCCTTTTCGCCTCACAACTGCAGCCTTTAACGTTTCCTCCTCTGGTCTGAGCCTGAGACGCCCGA
GTTTTCCTCTTTAGCTCCCGTGCCCCATTCCAGCCCCACCACAAATCCCGTGCCCACTCTTTCCACT
GGCCCAGGCCCAGCTCACTACCTTTCTTAGAGCCCCTTTCCGACTACGCTCCCTTCTGTCCCTTCTC
ATCTCCTCCCCCAACTCCTCATTCTGTTCCTGTCCCCAGAAACCATCCCGAAGTATCTCCCTTCCCT
CCTGTGGCTTCTCCCTATTCTCAGCCCTCTCCCACCCTAGCCCTCTCGCTATCACCAGCCATTAGC
GCACCTTTCCTTCTCAGATCCGTCCATACATCCCTCCCTCTCCATCTCTGGCTCGACACCCGGGCT
CACTATTAGTTCCCCTACATCCACCCCGCAGCCTGCTCTTAACCTCTCCTCCCCGGCGGCTCAGAC
CCAATTCTCAGTGTCCTTAGCGCCCCCTTCGGGCCACAGCGTTAAGCCACGCCCCCCGGGCCCCTC
ATCCGTTGCCAAGTTCGCTGAGCGTCCGCGTCTGGTTGCCTCTCCGCCCCACAGGGCATGTTCGTG
CTGGGCCTACCCTACGCCATCCTGCACGGCGGCTACCTGGGGTTGTTTCTCATCATCTTCGCCGCC
GTTGTGTGCTGCTACACCGGCAAGATCCTCATCGCGTGCCTGTACGAGGAGAATGAAGACGGCGA
GGTGGTGCGCGTGCGGGACTCGTACGTGGCCATAGCCAACGCCTGCTGCGCCCCGCGCTTCCCAA
CGCTGGGCGGCCGAGTGGTGAACGTAGCGCAGATCATCGAGCTGGTGATGACGTGCATCCTGTAC
GTGGTGGTGAGTGGCAACCTCATGTACAACAGCTTCCCGGGGCTGCCCGTGTCGCAGAAGTCCTG
GTCCATTATCGCCACGGCCGTGCTGCTGCCTTGCGCCTTCCTTAAGAACCTCAAGGCCGTGTCCAA
GTTCAGTCTGCTGTGCACTCTGGCCCACTTCGTCATCAATATCCTGGTCATAGCCTACTGTCTATC
GCGGGCGCGCGACTGGGCCTGGGAGAAGGTCAAGTTCTACATCGACGTCAAGAAGTTCCCCATCT
CCATTGGCATCATCGTGTTCAGCTACACGTCTCAGATCTTCCTGCCTTCGCTGGAGGGCAATATGC
AGCAGCCCAGCGAGTTCCACTGCATGATGAACTGGACGCACATCGCAGCCTGCGTGCTCAAGGG
CCTCTTCGCGCTCGTCGCCTACCTCACCTGGGCCGACGAGACCAAGGAGGTCATCACGGATAACC
TGCCCGGCTCCATCCGCGCCGTGGTCAACATCTTTCTGGTGGCCAAGGCGCTGTTGTCCTATCCTC
TGCCATTCTTTGCCGCTGTCGAGGTGCTGGAGAAGTCGCTCTTCCAGGAAGGCAGCCGCGCCTTTT
TCCCGGCCTGCTACAGCGGCGACGGGCGCCTGAAGTCCTGGGGGCTGACGCTGCGCTGCGCGCTC
GTCGTCTTCACGCTGCTCATGGCCATTTATGTGCCGCACTTCGCGCTGCTCATGGGCCTCACCGGC
AGCCTCACGGGCGCCGGCCTCTGTTTCTTGCTGCCCAGCCTCTTTCACCTGCGCCTGCTCTGGCGC
AAGCTGCTGTGGCACCAAGTCTTCTTCGACGTCGCCATCTTCGTCATCGGCGGCATCTGCAGCGTG
TCCGGCTTCGTGCACTCCCTCGAGGGCCTCATCGAAGCCTACCGAACCAACGCGGAGGACTAGGG
CGCAAGGGCGAGCCCCCGCCGCGCTTCTGCGCTCTCTCCCTTCTCCCCTCACCCCGCCCCCACCAG
CCCAGTGCGCCCTGCCGCCGCG
CGI: 315 range = chr20: 38749965-38753401
CGCTGGTGGAGCCCGCCTCCCTGAAGCCGGAGTTGGCGAGTTTCTCGCACTTGACCTTGTAGGCG
TCTCTCTCGCGGGCCAGCCGGGACACCTCCTGCTTAAGCTGCTCCACCTGCTGAATGAGCTGCGTC
TTCTCATTCTCCAGGTGGTGCTTCTGCTGGACGCGTTTATACCTGCAAGACTGGGCGTAGCCCCGG
TTCTTCAGGGTCCGCCGCTTCTGCTTCAGGCGGATCACCTCGTCCTTGGTGAAGCCCCGCAGGTGG
CGGTTCAGCTCGCGCACGGACATGGACACGAGCTGGTCGTCGGAGAAGCGGTCCTCCACGCTGCC
GTTGCCGCCCGCCGCCGTCGCCGAGGCCGTCGCGTGCGGCCCGGGCCCGGGGTGGCTAGTGGGC
AGCTGTTGCGCCGGGCTAGCGGCGCTGGACGGCGGCGGCGACGCTTGGTGATGATGGTGATGGT
GCGGGTGAGCGTGCGGGCCCAGCTCGTCGTGGGCCACGCCGGCGCCCGGGTACGCGTGGTGCGG
GTGAGGGTGGTGGTGATGGTGGTGGTGGTGAGCGCCGCGAAAGCTGTCGAAGCTTTGCAGCGGC
TGTGGCACTGGGTGCGAGCCGATGAGCGCTTCCACCGCGTCCTCGGGCGTCAGGTTGAGCGCCTC
GGGGTTCATCTGCTGGTAGTTGCTCGCCATCCAGTACAGATCCTCGAGGTGTGTCTTCTGTTCGGT
CGGGCTGAAGCTGGGCGACGAGGGCACGGAGCTACACGGAGTGCTGAGCGGTGTGGAGGACACC
GAGCCGGCTGGCTGCAGGCGTGTGCAGGGCCTGCCCGGACGCTCCGCGCGCCCCAGTGGCTCCTT
CTTCACGTCGAACTTGAGCAGGTCGAAGTCGTTGACATACTCCATGGCCAGCGGGCTGGTGGGCA
GCTCTGGCCCCATGCTCAGCTCCGCGGCCATCGCTGAAGCGAGGCGCAGCCGCCGCTGCCGCCCG
GGAAACTTTGCGGCCGGCCGGAGCGCGCCGAGCCAAGCGCGGGGGGGAAGAGCGGAGAAGAGC
TGGGGAGGCGGGGAGCGAGGGCGCAGCGGGCCGGGGCCGCCGGCCAAGCCTTTGTCTGGGGACG
CGGCGGCGCGCCGGAGAGTCCCGAGGCTGCCTGCACCGCCCCAGAGCTCTGGGCTGTGCCCGCG
CAGGGACCGGGCCGGGTAGAGTCGGGCGGGGTGGAGAGGCAAGCGGAGCGCGCGGTGGGGCTG
AGGGGAGGCGTGGGGCGAGTGCCCGTTGCTCGCTCTCTAGCTCTCTTGCTCTTACGCTCTCTCGCT
CGCAGCCGCTCGCAGCTCGGCGGTGCAGCTGTGCTGGATCCGGCGGCGCCGCAGCCTTTTATCGC
CTCCTGATGTCACTGGGGTGCGGGGGCCCGGGCGGCCCGGTGCGCGGGCCAATAGCTGCACGGC
CTCCGCGGCCCAGCGGCGCAGGGCGGGGCGCGCCTGACAGCTCCCCCGCCCCCCGCGTCAGCTG
ACTGGCGGCCCGAGCGGCCCCGGAGCGGCGGAGGCCTGGCGGAGCGCTGGAGCGGAGTGGGAC
GGCCAGCCTGGGCCCACCCCCGTACCCTGCAGGTCCCGGCCCACGCACGCTCGCCTGGAGTGCGC
GCCCCACCTCTAGGCCAAATCACCGCTTTCCCCTCCTCGCGCACTCTCCTCCCTCAGTTCCCTTTGC
ACCCCACCCCCATCCCGTGTCACCCCCAAGGAGGCTCAGAATGAGCGCCGGGACAACGCCTCCTG
GGCCCTTTGTTCCCAAGCGGCCCCCGCCCAGTGGGCGACGCTCTGTGTGTCCTCGCGGCTTCTGGC
CGTGTGTGTCGTGCGTTCCTGTTTCTGGAGATCTGCGCGTATTTGTATGTTGGGGAGGGCGGGCTC
GAGGCTCCGAGAGTTGTGTTCAGACCCAACTCTTAACCTCAGGGGACCTTTCTCAGGCCAAGCGA
GGGCCCCTCCTGGCGGGTGCAGTCGCAGAGCCCTGAGGTTCGACTCCACTGGCCCCGCCGCTCCC
CGCGTTCACCCCACCGCACAATGTTCACAGTGAAGGCGACGGGAAAAGCAGCAGCCCAAAGGCT
CTGAATTCCTCTTCCCCGCCACACGCACGGAATCCTGAGCCCCCGGAGCCTCGGGGCCGAGGCCG
GCCCGGGACGGTGCTCCGAGTAGCTCTCCACTGCTGGGGAGCCGGCCCTGTTTTTGTTTGAACGTT
TTGTAACGATTAAGCAGATCCCGGCGTCAGCCCGCCGCGGAGAGGCTCAAACAGGCATAAAGTG
CGACCCCAAGTGGCCACTGTGCGCAAAGGCGCCGCGACCGCCCGGCCCACGGCCGGAAGGCTTG
GACGGCGCCTCGTACCCAGCCAGGTCTCCCCTACCTGGCCCAACCCAAGCCAGCCCAGAACGCAT
ACTATGTGTGCACCAGAGCCCAGGACAGGTTCCCCTCGAGCGATGTACAGGTCCTCGGGTCCCGT
CTTCGTACTCAGCCGCGAGCCTCGAGCCGCGAGCTCCGCTCTGGTCGCCCCGTTGAAATTCCGTG
CCCCAGCGTTCGGGGGTGCCCGTCGGCTGCTCCCTGGGCCGGAAGGTCCTGGGCGGAGGAAGGC
CGGTAGCCAAAAGTGGAAGCGCCACAGTGAAGCGGCCCAGGGCCACCGGGTGAGAAACCTCCCC
GGAGGGCAGACGGGGAGACCGAAGCACACCGCACTAGGCATCCAGACTGGGCTTGGGAGCCGC
GCACCCTCCCTACCCAGATCCAGGATGGCTAGAATTAACGGGTTCTTTCTGAGACCTCGGCTCAG
GCGCCGAAACCGGATAGATCGCGAATTCGCTGGACCCGGAGACCCGACCCGCCTCCCGCGTCAC
CTTCTTCTTTCTAGCTTTGGGCGCGCGCAGCGAAAGGCAGGAGAGGCGCGCACTGGGTGAGTGAG
TCCCGGCCGCTGTCTGCGCTGGACCAGCCCGACTGACCTCGCGCGTAGGGGTCGCGTGAGCCACA
CCGGTGCAGACGCGCCTAGATTATTTTTAAATGTTAGAAGGTAAAATATTTGCCTCCAATTAATCT
GAAAACTCTCTATTCTCTTGCGCCCTCGGAGAGGCTGGGGTACGGCGTGGTATTGGGCCGCCTAT
TTTTAATAAAATGAGTGTATTTTAACTAAAACTTAACTCAATCTTGTGGGGGGCAAATTAAATGC
TGGAAGAGCGCGTCTACAACCCTCTTCGAGAAGCGTGCTCTCCGCAGAAATGAGTCGGCCGCCTG
GAGAGAGAGCCTGGGCGGTGCCGCTGCGCAGCCCCTGCCAGTAGCTGGGGGTTGGGGACTCGCA
CCTTGTAAATGTCCTCGTCTTGTTTGAACGCAGTGAGAGCACACTCGTTTCCAGATCACTCGGGAC
CGGGTGTCTCGGATCTGTGCAGACTATGTATGGCTCCGGCCTCAGGCGGCCAGGGCGGGACAAGC
ACG
CGI: 43 range = chr20: 49542203-49542863
CGCTGGCACCACCGAGCCCCGCCGCACCCCGCCGCTATTCTCAGGACGACCTGGGCAGCCCCCCA
GCTTCTAGAAAAGACACAGAGGAGACTGCGCAATCAACAGGCAGGAGTGTGCGTGAGTGCGGCA
TGACATGGGCACGCCACCCCAGAGAAGTGGGCGCCTGCTGCGCTAAACGTCATCCGTCATCCTTT
CGCTGAAAAGTTGGAAAACAAACAACAAAAACCCCGCCCAGCCTCATTTAATGCGAACCAAACA
CAAAAGCCACAGGGGAAAGCTTTTCCTCTCTGTGCTTCCCTTTTCCGGCCGAGGAACACCACCAG
CCAAGATAAGGGGACTGTGCGGGGCCCTCTGCGCGGCTTCAAAGGGCTGGCACCCGCCCGGGCC
CGCTCCCACTCCCACTGGCTAATGGGCTCCCCGCTCCAGCCCTTGGGGAGCAGGAGGCGGCCGGG
GCTCCATGCCAGGCGACTTGGAAGAGGTTTAAGGCTCACGCCACACAAGGGAGGAAGCTCGGCC
TGTCAAGAGGCGTGATAAAGCAGCCCCTTATCGCAGTTTTCCTTTCAACTGCCTCCAAGTGAGCG
CCGGCCGGGGCTGGGCCTGTCAAGGCCAAGCGTGTTCCTTTCATGTCGCCCTGGGTTCCTCGGTC
AGCGACGTATCCACG
CGI: 20 range-chr20: 54411932-54412178
CGCGGGTTTAAGTGGACGCCAGGTGCACCGGACACAGGCTGTGTTTAACCACACCGAGGACTAT
GTGTTGCTGCCCGACGAGAGGACGATCAGTCTTTGCTGCTGGGACTCGAGGACAGCCGAGCGGA
GAAACCTGCTGTCGTTGGGGCACAACAATATTGTACGCTGCATAGTGCACTCCCCCACCAACCCC
GGGTTCATGACGTGCAGCGATGACTTCAGAGCGCGGTTTTGGTACCGGAGATCG
CGI: 30 range = chr20: 55680656-55681057
CGCACGCGCACGTGTGTGCCTGCATGCACTCACATGTGTGTTTCCAACCGGCCGACAGTGACGCA
CGTATGCCCTCGCCACTCGGCAAGCACCTCGTGCTCCTTGGTAACCAGCAGATGTTTCTCTCAATT
ACTGATTTTCAAGAGCCTGGTGTTTCCTGACATGAGCCAGGATGGCGGGCGGCGCCCAGGGGAG
GGGAAGTGAACCGGGGCTCCGTTTCCAAGGCTCTTGGACAAAAAGCGAAGCCCTGGATCAGGAA
GCACAATGCGGCTGGTATTTAGCCGCTTCTCAATAGTGCCTCATTCAATCCCCGGGGCCTCATTGA
TGCCCGCCGCGTGCTGGAGAGCCTCTGAGGGTCAGTCGGCGACGGGCACCGTCTGGAGGGGTCTC
GCCGCTAACCCG
CGI: 26 range = chr20: 56860125-56860442
CGCAGCATGGTAGCGGGAGCGCAGCTGCGCGGGCAACCGCCGGGTTTGTGGCCGGTTGGCGGGC
TCAGTGCCGCCTCAGTGGGCTCTAGCAGTAGAAAAGTAAAGCGACTACCCTAAGATACCCAGAG
CGAGAGGAGAGGGGCTAGAAGGGTGCTGGAGGCAGGCTGTAGTGGGGCTAAAGGAGCTGACTG
ACCAGCGCTGCGGCCGGTGCCCGAAGCAGCGAGCAGCGCACCTCAGCTCCGGTTGCCGCGCGGC
ATGGTATTTATCTGTGGGTTCAGTGCGCACAGCGTTGTCCAGTCTCGAAACCACCCTAAACCG
CGI: 18 range = chr20: 62371744-62371958
CGTGAGTGAGGGCGTGCACGGTGTGGGGTCCTGTGAGTGCGGGTGTGCACGGTGTGGGGTCGTA
AGTTCAGGCGTGCACGGTATGGGGTTGTGTGAGTGCATGGCGTGGGGTCTTGTGAGTGCGGGCGT
GCACGGTGTGGGGTCTTGAGTTCAGGCGTGCACGGTATGGGGTCGTGTGAGTGCATGGTGTGGGG
TCGTCAGTGCGGGCATGCACG
CGI: 22 range = chr21: 45511336-45511549
CGCAGGGCCCACGCTCCCGGCCTCTGGGACGCTCACGGCAGCCCCACGAGAATGATCACACGAG
GGCCCACGCTCCCGGCCTCGGGGACGCTCACGGCAGCCCCACAAGAACGATCACACGAGGGCCC
ATGCTCCCAGCCTTGGGGACGCTCATGGCAGCCCCACGAGAATGATCACACGAGGGCCCACGCTC
CCGGCCTCGGGGACGCTCACG
CGI: 24 range = chr21: 46892946-46893236
CGCGGCCTAGAGCGGTGAGTGGGGTCTCGAGCGCATCCCGGGTGTTTGTGCCGAGGCTGGTGACG
TCCGAGGTGGCCTCTGAGTGTGCTGACTTGTGACCCTGAGCTGTTGGGGGCTCACCGGTGACTCC
ATGGTCTTGTTGAGCACCCTGCACGTGGGGCTCAGGGTCGGTAAAATAGCAGTGCGTGGAGACCG
CGTGCTAGAGGCCGTGGCGCCCGCGTACAATGAGTCGCAGACAGCACAGACGGGAGTAGGGCAG
AATAGACAATATCCCGTGAATTGCGTGGGGCG
CGI: 38 range = chr22: 18453364-18453931
CGCCAGCGCACAGCGCGGCAGGACGCGCCCGGGTCTCAGCGGACTTGTGCATGTTAGCTGTGTAG
ATTTATGTGAGGGCTTGTAAAACTCTGGTCTTGTAAACTAGTCTTAAGCGCTTTTAATATGGAGAC
AGATGAGAGCCCCTCTCCGCTCCCGTGTGGGCCCGCAGGAGAAGCGGTGATGGAGAGCCGAGCT
CGCCCCTTCCAAGCGCTGCCCCGTGAGCAGTCTCCACCACCTCCCCTGCAAACGTCCAGTGGTGC
AGAGGTAATGGACGTTGGCTCTGGTGGTGATGGACAGTCCGAACTCCCTGCTGAGGACCCCTTCA
ACTTCTACGGAGCTTCTCTTCTCTCCAAAGGATCCTTCTCTAAGGGCCGCCTCCTCATAGACCCGA
ACTGTAGTGGCCACAGCCCGCGCACCGCCCGGCACGCACCTGCGGTCCGGAAGTTCTCCCCTGAC
CTTAAGTTGCTTAAGGATGTAAAGATTAGCGTGAGCTTTACCGAGAGCTGCAGGAGTAAGGACA
GGAAGGTGCTGTACACAGGAGCAGAGCGCGACGTGCGGGCGGAGTGCG

TABLE 8
PcG target genes methylated in all bladder tumors or specific subgroups.

| | NMI-wt | NMI-mt | MI | NMI-mt and | MI and | MI and |
|---|---|---|---|---|---|---|
| All | only | only | only | NMt-wt | NMI-wt | NMI-mt |
| DLX5 | ADCYAP1 | BARHL2 | TBX3 | CNTNAP1 | EVX1 | HOXA5 |
| DLX6 | ATOH1 | BCL11B | | EBF2 | GATA2 | |
| FOXD2 | BHLHB5 | | | FOXA1 | HOXB5 | |
| GAD1 | C20orf103 | | | FOXD3 | MSC | |
| HLX1 | CACNA2D4 | | | HHEX | NKX2-8 | |
| HOXA7 | CBLN1 | | | HOXC4 | SIM2 | |
| HOXA9 | CBLN4 | | | HOXD3 | VAX2 | |
| IRX3 | CDKN2A | | | MAFB | | |
| MAB21L1 | COL12A1 | | | ONECUT2 | | |
| MEIS1 | COL27A1 | | | OSR1 | | |
| MSX1 | CYP26B1 | | | PCDH17 | | |
| MSX2 | DMRT3 | | | PCDH7 | | |
| NR2F2 | DPP10 | | | PITX2 | | |
| NXPH1 | ELAVL2 | | | SIM1 | | |
| OTX1 | EMX1 | | | SIX1 | | |
| PAX9 | EN1 | | | SOX7 | | |
| PITX1 | FBN2 | | | UNG2 | | |
| PRKCZ | FEZF1 | | | ZIC1 | | |
| SHH | FLJ46347 | | | ZNF503 | | |
| SIX3 | FOXA2 | | | | | |
| SOX21 | FOXB1 | | | | | |
| TFAP2A | FOXF1 | | | | | |
| | FOXG1B | | | | | |
| | GATA4 | | | | | |
| | GNAS | | | | | |
| | GRID1 | | | | | |
| | GZF1 | | | | | |
| | HAND2 | | | | | |
| | HEY2 | | | | | |
| | HMX2 | | | | | |
| | HOXA11 | | | | | |
| | IRX1 | | | | | |
| | IRX5 | | | | | |
| | ISL2 | | | | | |
| | ISLR2 | | | | | |
| | KCNK9 | | | | | |
| | LBX1 | | | | | |
| | LBXCOR1 | | | | | |
| | LHX4 | | | | | |
| | LHX9 | | | | | |
| | LMX1A | | | | | |
| | LRAT | | | | | |
| | MAP1D | | | | | |
| | NFK | | | | | |
| | NKX2-2 | | | | | |
| | NKX2-3 | | | | | |
| | NKX6-1 | | | | | |
| | NPAS3 | | | | | |
| | NR2E1 | | | | | |
| | NR3C2 | | | | | |
| | NR4A3 | | | | | |
| | NRN1 | | | | | |
| | OLIG2 | | | | | |
| | ONECUT1 | | | | | |
| | OTX2 | | | | | |
| | PAX5 | | | | | |
| | PAX6 | | | | | |
| | PCDH1 | | | | | |
| | PCDH10 | | | | | |
| | PHLDB1 | | | | | |
| | PTGER3 | | | | | |
| | SEMA6A | | | | | |
| | SFMBT2 | | | | | |
| | SIX2 | | | | | |
| | SIX6 | | | | | |
| | SLC32A1 | | | | | |
| | SNRPF | | | | | |
| | SORCS3 | | | | | |
| | SOX17 | | | | | |
| | SOX9 | | | | | |
| | T | | | | | |
| | TBX15 | | | | | |
| | TBX20 | | | | | |
| | TBX5 | | | | | |
| | TFAP2B | | | | | |
| | TRIM2 | | | | | |
| | TRIM36 | | | | | |
| | TWET1 | | | | | |
| | VIPR2 | | | | | |
| | VSX1 | | | | | |
| | ZIC4 | | | | | |

TABLE 9
CpG islands (CGIs) methylated in blood but not in bladder cancer. Provided for each
are: ID no.; Coordinates of selected regions containing relevant CpG islands based
on Homo sapiens full genome as provided by UCSC (hg18, March 2006); GeneName;
Location of the probe used; the Fold Change; and the sequence of the CGIs.

| No | CGI | Gene Name | CGI probe location | FC |
|---|---|---|---|---|
| 1 | chr19: 53930240-53930625 | RASIP1 | chr19: 053930330-053930374 | 2.66 |
| 2 | chr7: 72883371-72883981 | CLDN4 | chr7: 072883457-072883501 | 2.64 |
| 3 | chr22: 26522794-26528592 | MN1 | chr22: 026525238-026525282 | 2.21 |
| 4 | chr7: 5614184-5614510 | FSCN1 | chr7: 005614143-005614190 | 2.15 |
| 5 | chr16: 66239477-66241425 | RLTPR | chr16: 066241103-066241147 | 1.93 |
| 6 | chr5: 551112-551430 | SLC9A3 | chr5: 000551380-000551424 | 1.86 |
| 7 | chr19: 6426223-6426998 | DENND1C | chr19: 006427051-006427102 | 1.82 |
| 8 | chr3: 32836146-32836433 | TRIM71 | chr3: 032836466-032836511 | 1.78 |
| 9 | chrX: 129301145-129301626 | SLC25A14 | chrX: 129301313-129301357 | 1.76 |
| 10 | chr9: 124027565-124030907 | LHX6 | chr9: 124029663-124029718 | 1.74 |
| 11 | chr10: 129424401-129427356 | FLJ46831 | chr10: 129426989-129427033 | 1.71 |
| 12 | chr1: 3147370-3148160 | PRDM16 | chr1: 003147944-003147988 | 1.65 |
| 13 | chrX: 128941527-128945710 | BCORL1 | chrX: 128944842-128944886 | 1.64 |
| 14 | chr17: 7584523-7585036 | DNAH2 | chr17: 007584859-007584908 | 1.63 |
| 15 | chr1: 27548507-27550603 | SYTL1 | chr1: 027550188-027550232 | 1.6 |
| 16 | chr19: 10304689-10307022 | ICAM3 | chr19: 010306004-010306051 | 1.56 |
| 17 | chrX: 129132628-129134108 | AIFM1-RAB33A | chrX: 129133196-129133244 | 1.55 |
| 18 | chr21: 45648960-45650662 | COL18A1 | chr21: 045663489-045663539 | 1.53 |
| 19 | chr10: 134864694-134865392 | KNDC1 | chr10: 134865054-134865098 | 1.48 |
| 20 | chr9: 129556229-129557670 | SH2D3C | chr9: 129556146-129556190 | 1.48 |
| 21 | chrX: 47305347-47306043 | ARAF | chrX: 047305953-047305997 | 1.45 |

Sequence of the CGIs of

1 chr19: 53930240-53930625
CGTGCGGGGCTGGGGCGGCGGTTACCTGGGCGTCCTGGTAGCCCTGGAGCAGCAGGAAGTAGGGGCGGTTGCTGGGGGCC
TGGATGAGGCACTGAGTCAACTGATCGAAGTCCCCGGGGTCTGCAGTTCCGATTTGGGCGTCGGCTGCCCCTGGGGCCATG
CTAAGTGCCTGCTGTCTCCGCTCCTGCTGCCGCCGCCGCCGCCCCTGAAGGCTAAGCTCCGACACGCTGCGCCGCAAAGAC
AAGTTTTCTGAGCGCTCCTTGCCTCCAGACCCAGCTGGGGCCCCTGATCCGGTCCCCGGGCCAGGACTGGCCAGCGCTGCCC
CACCCGACGCCGCCCGGGAGCGGTTCTTCTGTGGCCGCCACGAAGGGGCGCCGGTGCCTGCG
2 chr7: 72883371-72883981
CGAACGTTAAGTCCGTCCCCAGCGCTTGGAATCCTACGGCCCCCACAGCCGGATCCCCTCAGCCTTCCAGGTCCTCAACTCC
CGTGGACGCTGAACAATGGCCTCCATGGGGCTACAGGTAATGGGCATCGCGCTGGCCGTCCTGGGCTGGCTGGCCGTCATG
CTGTGCTGCGCGCTGCCCATGTGGCGCGTGACGGCCTTCATCGGCAGCAACATTGTCACCTCGCAGACCATCTGGGAGGGC
CTATGGATGAACTGCGTGGTGCAGAGCACCGGCCAGATGCAGTGCAAGGTGTACGACTCGCTGCTGGCACTGCCGCAGGAC
CTGCAGGCGGCCCGCGCCCTCGTCATCATCAGCATCATCGTGGCTGCTCTGGGCGTGCTGCTGTCCGTGGTGGGGGGCAAGT
GTACCAACTGCCTGGAGGATGAAAGCGCCAAGGCCAAGACCATGATCGTGGCGGGCGTGGTGTTCCTGTTGGCCGGCCTTA
TGGTGATAGTGCCGGTGTCCTGGACGGCCCACAACATCATCCAAGACTTCTACAATCCGCTGGTGGCCTCCGGGCAGAAGC
GGGAGATGGGTGCCTCGCTCTACGTCGGCTGGGCCGCCTCCG
3 chr22: 26522794-26528592
CGCCAACGTCTTGTCGTCGTCCGCGCTGTCCACCAGGGCCTTGTCAGCGGGCATGTACCAGGCAGCGCTGTGCTCTGCCATC
AGCGAGTCCAGGTCAATGGTGCTCATGGCGCTCTTGACCGCCTCGGAGCAGCAGCTGCCCAGCTCGCTGTCGCCATTCTGCG
CCCCTGAGGCCCCGACGGCGCACTCACCCTTCTTGCCACCCTTCAGCCCCAGAGGCTGGTCCTCGGAGATGCTGAACTGCTG
CCTCTGTAGCTGGATCTGCGCCTGAAGGATCTCCAGGGGGTGGATCTCGTCGGGTGGCGGGGCGCCGCTGCTGCTCGTCGG
GGTGCGGACCTGCTCCAGGCCCGGAGTGCCCGGATGGCCCGGGCCCCCACCGCCGCCGTAGCTGTCAGGGGTCGAGGTAGA
GTTAGACATGATGCCCAGGCCGAGGGCGGGCGGGGGCGCCTTCGGTCCGTGTTCCCCGGCGCCTACCCCACGGGGAGGGA
GTTTGGGCGAGCCGGTCACCAGGGGACTCCTGCTCGCTTTAACTAGTGCCTGGGGGTTGTCAGAGCTGGACGACACCTCGT
CCTCATTGGCGTAGCTCGTGCTCACCTCGTCCGAGGCGAACTCACCCACGTTTGGCGAACTACTGTCCGACTTGGCCCCGCC
GTCCAGGGACCCAATGAGGTCCGGCTGATCCCCCAGGAGCAACTCAGCCCCCTTCCCCCAGGATGGCGACGTGAGCGCCTT
TTCGTGGGGCGTCGGTGCCCCGCGCGTCTCGCCTGCGGAGCTTCCCCCGACGGCTGCGCCTGACGCTTGCTGCTGCCCTGGG
CTCACCCCAGGTGCGCCCCCGCTGTCCGGAGCCGCCGAGTACTTGTCAAAGAAGGTGCCAGGGCTCACGTGACCACTGTCC
CTTTTTCTGCGACCCCGTCCCCGGCCGCCGCCCCCGGAGACCGGCTTGCCGTCATTCCCCGACGTGGATTCCAGGGTGTAGT
TGGGGGAGAGGCTGGTGCCGTCCCCCTGGGCTGGAGGGTTGGGCGGCCCCGAGGCTTTGGAGCCGCTGCTACTGGTCCCGG
ACGGGCCTCCGGGTCCTGGGGCCCCAGGAGCAGTCCCTCCTGGGAAGTAATCCGAGCCCGGGTTGCCGGCCACTGCCGCGC
CGTCGGTCTCGTTCTGGCTCAGTTTCCTCTTGCCCTCTGGCGGGTTCTTCTTGTTGAAGGTCACGTTGAGGTTGGGGGCCCCG
AGGCTGGCGATCATGTTCTGGCAAGCGGTGGAGAGCGCAGCCAGGCAGCTCTGGCCGAACAGGTTGTCCTTGGAGCTGGGC
TTGTTGAAGGAGCCCAGCGAGAGCGCGCCCAATTTACTGGCCGAGGTGCGCTGGCTGGGCTGGAAATCAGGCTGCGGCGG
GTAGGCACCCCCGCCACCGCCGCCACCAGAGCTGCCACCGCCCCCTCCCGCGCTGGGGGGCGAGTTCACGCCTGGACCGCT
GTGCGGCGTGGACTGCCGGCCGGCTGCACCAAACGGAAAGCCCGGCTGGCCCCCGAGCGCAGACGTAGCAAAGTCCGGCG
GCGGGGGCCGGCGCTCCGAAGCAGCGCTGGGGAGCCCCACGCCCGCCCCGGGCGACTGCAGCTGACCCAGGCCTCCCAGA
CTGCCCCCGAACTGCAGGCCCGGTGAAGGCAGCGCGGGCACGTGGCCCTCTCCGGGCATCCTCATCGGCTCCTGCAGAGGG
CCCCGGAACAGCACCCCCGAGCCACCAGGCGGAGGAGGGGGCGCCAGGCTGGGGTCGTGCGGGCCACAGTCAGCGGGCAG
ACCCGAGCCGCCCATCCTACGGGGCAGCAGGTCTCCGGGCGGCGGATGCGGACCTGAGAACCACGCGCTCTCTTGCGCCAA
GTGCGGCGCCTGCTGCTCGAAGGTGCCCAGACGCCCGGCGCCCGTGCTGCCGCCTTCGCGCTCAAAGTTCGGCTGGGCCAA
GCCGCCCACCGGGCCGCCATGCACCAGGCCGCCCTGGCCCACGTCCCCGGGGTGGCCTAGCTGAGCCAGGTTGGGCTGGCG
CAGCCGCTGCTGCTGATTCCGCGACGCCATCTGCTTAATCATGAGGGCCGCGTTTTGGCGCTGCTGCTGCTGCTGCTGTTGC
TGTTGCTGTTGCTGCTGCTGCTGCTGCTGTTGCTGCTGCTGCTGCTGCTGCTGCTGCTGTTGCAGGGACTGGTGGTCCGGGGC
CGGATGCTGCAGGGGCGGCCCCGAAGGGAAGCTGTCGGGCACAGGCGGTGTGAACTCGCCGGGTAGGCCTGGGTAGGCGG
AAGGGGAGAGGTGATTATCCAGAGCGCCGTTGTGCATGCTGCCGTTCCACGAAGCGCAGCGGTCCACTCCCGCGCTGCCCG
GAAAGTCGAAGCGCGGCCTCTTGGCCACGTTCATGTAGGGGGGCGCGTCGAAATGCTGCAGCCGCTGGTTGGGCGCCTGCT
GCGGAGGAGGATGCTGCATGCTGAAAACAGGCTCGGAATAAGGGTGCATGCTCCGGTTCTCCAGCCGGTGGATGGGATACT
CGAATTGCGCGTGCTGGCTGGGCAGCATGGGGCCTCCGTCCTGCAGGCCGCCGCTGGGCGTGCCCGCCTCGCCCTGCTGGG
GCCGAGGGAGCGCAGGCGGGCACGAATTTTGTCGGACTAGAAGCCCGGGTGGCGGCGGCGGCTGCTGCTGTGGCGGCTGC
TGCGGGGGCTGCTGCTGAGGGGGTGGCGGGGCCTGCTGGGGAGGCTGCATTAACGGGTGCCTGGAGCCCACTGAGGGCTC
CAGACCCACAGGCATCTTTCTGGCCCCACTGAACCTCTCAAAGAACACACCATGCTGCTGCTGCTGCTGCTGGGGCTGCTGC
TGCTGCTGGGGCTGCTGCTGCGGTGGCTGGGCGTGCATTTTGGACAAGCCCACCATGCCCGCAGCTCTGGGCATGGCCGAG
GCGCCCGGGAAAGCGCCCCCAGGAACCTGGCGACCCGCTGCATAATGAGGCAGCTGCCCTTCGGAGTCAGAGGGCGAAAA
CATGTCAAAATGTCCCGAGGGCGCCTCGCCCGGGTAATTGTATTCCAGCGAGTCGACGGCTCCTTGGTTCGTCACCCTCCGT
GGCTCCAGACTGTGGGAATCGGAGCCGCTGGAGGACGGCAGGCCGTGGAAGGAGGCGGCTCGGTTAGGGCTCTGGTCCAG
CGGCAGGCATGGGGCCGGCACGGCGTGGCTGGAGGCACCTGAACTGTGGAAGTCCGGGAGGTTCCCCGGTCGCTGCGGGC
CGAAGCTCTCAGGCCCCTGGCTCTCCGCCATGTGCTCATAGCCCTCGGCGAAGGGCGGCTGGCTGCCCAGGCCTCCGGCTG
CGCCGCCGTAGCCGAGCAGGCGACCCCCGTGCAGGCACGAGGCCCCGGGGTCCGGGCCACCGAAGTTGCCCCCAAAGTGG
GGGTGATGCTGGTGGGGATGATGACTTCCCGGGTGGCCGTGGTGAGGCTGCTGGCCGCCAAAGAAGCCGTGCACAGGCTGC
GCTTGCAGCCCCCCTGCGTGCAACTCCGAGTGGCCGCGCGCGTGGAAGCCGTAGGGCTCCATGTTCATGCCCAAGATCGGG
GGTTCGCCCAGCGCGCTCATAGCAGGATCCACAGGGCCAGGGGGCCCCCCAGTGTGGAAAGCCGGGGCCTTAAAGTGGGT
GTTCATGCTCAGTCCGGTCTCGTTAAAGTTCCTCTCGCCCTGGCCAGCGTTCCTGCTGTTGACCTGGGGCTCGAATTGGTCCA
GCCCAAACATACTTGGCGGGGGGCAGAGGGGGATCAATAGGGCATGACAGCCGGCTCTCCGCGGCGCGCCTCCGGCCAGC
TACTCGTTCCAGCCCAGGATTGGGCGCTCCGGGACGCTCAGCACCGCGGGGGCTCAGCGCGCACCTCCACCCCGCCTGATG
TGAGGGACGGGGGGCGGGGTATTAGCTCCTCTCCTGAAGCTCCGATTCTGCCCGGGGAGGGCCTCTCACATCTTGCGAGGC
CGCGGGGCCTCTAGGAGCCGTGTTGGGGGGCCCATGCCCCGGGCGGTTGTCACAGCCGCGGGTGGGTCTGCGGGGAGGGG
ACGAAGCCGCGGATGAACGGAGACAAAAAGTTAAGTGGGGGGAATGGGGAGGGAAGGGGGTTGGGAGAGCAGAGCGATC
ACCTTCTCAAGTCCGATTGGGTCTGCTGGGGAGCCCTCAGGACGCCGCCCGCAGCCTCCCGGAGTCCGTGGCAGAGCTGCT
AAGGGCAGGGGAGGGAGACCCTTCAAAGCCGCGGCTGGCGCCGGGCACCGACAGAGCGGTCCCTCCCCCCGCCCCCCGGA
GTCCCCGCGCCCCGCAGCCCGAGCCTCCACCGAGCACGATCCGCTCGCACAATCCCCGTAGGCTCCGGGCGAGCGGCTGCT
GCTTCTTCAGCGGGTCGGAGGACTGGAGGCTCCGCTCGGCATCGCCGGTGCCGGCCCCCGAGCCGAGGACGCAGAGGGGCT
GCGAGGCGGCAGGCGCCGGGGGCTGGAGCCGAGGGTCGGGGAAAGGCGCGGCTCCTCTGCTCGGCAGCGGGTGCGCTGCG
TTCGGCGCGCAGCTTCGCGGCCACGTCCGCCGCCTGCCGCTTCTGTTCTCCGCCGTTGGGTGTCTGAGTTCGCCGGAGTCTC
CGCGCACCGTTGCAGGCGCGGGAGGGCAGCGAGACGAGGGGTTGGGTCGCTCCAAGGCTCCGGTTCCCTGCCTCCGCGCTG
AGGTGCTTCGCGAGTCCCTCTCGGACCTGAGGGAGGGGGGCGTACGCGGGTCGGGGGGCCACGCCGGGCGAGCAGGCTAA
GGCGTTCCCGCTGCGCTCTCAGAGCCCGGAATGGGGGGAGGGGGGCGCGGGGGCAGCTCTGGGGGGTCTGCGCACCCCTCT
CCCGACTAGCGGGGGGGCTCTGCGTGGGGCGTTCCGAGGGTCTCGGCTCCCCTCTCTGTGTCTGCCTCTCGCCCAGCGCCGG
GAGAAGCAGCAACAAGTTTTGCATTTCAGCAATCAATTTCAGCCATTACATTTGCACCAATCAGCGCCGCCCAAGTTCCGG
GCTCGGGGCGGGGCTCGCTCTTAAGGTGGTCCGGGGTCCTGGCTGCCGAAGCCCCCGCCACGAACCCGCACTACCCCAGCT
CTGGGGTCCCCTGCGCCCGCCTCTCCTCGGGGTCCGGCGTCAGTGCGCTGGGGGCGCGCCCCGGCCTCCGGGCGCATCGAG
CTGGAGAGGGTGCAGGCGGAGGAGAATGGCGGGAGCTTGGCTTTGTTCACCCCCTTCCCATTCACACTGTTCCCAACTCCCC
ACCCCCAACTGGAGCGAGACCCACCGGGTGGGCAGGGGGGGGGAGGTGGGCGGTGAGCGGTACGGGGCGCTCAGACAA
TGAGGTCGCCAGGCAAAGACCCTAGACTCGCCACGGAAAGTCGCTAAGTGTCTACTGCACTTCGGCTCCGGGGTGAACACT
CCGTTCTCCGCTAGCGGCGCCCAAGTTAGCCGGCCCTGGGATTTTCTTGGAGGGTAAAGCAGACGGGGAACCTCGAAGTTA
GGGTTCTCCTCGAGAGACCTGGGCGCTTGCTCACTCCTCCGTCACTAGGGCGTTCACAATTGTTCCCCTTCCCCACTGCCTGG
GGTTCCCGGTTCTCACCGGCCGGCGTGGGCTCCAAGGAGTCACAGTCACTCAGCCACTTTATGGGGGAGCACATCGGTTGA
GCCCAGTAACCGGAGCGACTTGCGAGGCGGCTGGGAACCCCGAGAAGACACGCACGAAGAGGGCGCACCCGCCGGGCCGC
ACACCTTGCCCTGGGCCACCCGGTCCAAGACCCAGACCCTCTGCGGGTGCG
4 chr7: 5614184-5614510
CGCCTACCACGTGGCCGAGATTCCCGAGGCTGCAGCCAATGAGCAGCATAGTCCGATGGGGGAGGCTGGAAGGGCCGTTC
ACGAGATCTCTCGGGAGGAATCGGTAGGAGCTGCCCAACTGAAAGGGACGGGGGAATAACCCGGGGCGAGGGACCTGCCC
AGGCCCTGCCCTCTGGGGGGAAGCCAGGCCAGGGGCCGGGGGCATCTCTGGGGTCCCAGGTGAGATGCGGGTCGAGGGAG
CCAGGCCACGGTTCTCCAGACTCGCGTGGAGGCCACGAGCGTCTTCTGGAGGAGCGGGCACTGCGCGGACCGGCAGACTCT
GGGGCG
5 chr16: 66239477-66241425
CGTCCTAACGTACTGTCGTTCCTGAATCTCGCAGGCACCGACACTGCCCTGGACACTGTGAGGGGGTGCTCCGTGGGGGGA
TGGATGACCGGCAGGGCGGACTGGAGGGCGGGACGGGGAGGGCTCGGTCCCCCCGCGGGTGTAGCCAACAGCCTCCCCCC
GCAGCTCTTCGCAGCGGTATCCCGAGGCTGCTGCACCAGCCTTACCCACCTCGACGCTTCGAGGAACGTCTTCTCCCGCACG
TAAGGGGGACCTGTCGGGGCCGGGGGAGGCTGCTGGAAGCCGCCTCCTTGCGGCCCCAGGCCCACCTTCCCACTTCCCCCA
GGAAGTCCCGCGCCGCGCCGGCCGCGCTGCAGCTCTTCCTCAGCCGCGCGCGGACGCTGCGGCACCTGGGCCTGGCGGGCT
GCAAGCTGCCGCCCGACGCGCTCAGGTCAGTGTCGGACCCCGGCCACGCCCCCGCGGGCGCTCCCACCCTGCCCTGGCCTT
CGCCCCTCCCCGCTCCTGCTTCTGTCGCTCCCACAACCTCCCCCAGATCCTGGCCCTGCCTCCTTCGTTCGCACCCTGGAGCC
CCCTGTCCCAGCTCCCGCCACCCCGTGTAACGTCCTCTCCCAGAGCCCTTTTGGATGGCCTCGCGCTCAACACGCACCTCCG
CGACCTGCACCTGGACCTCAGCGCTTGCGAGGTGAGCGCCGGCCCCCAGAAGAGACCACACATTGGGAGAGGCGCTGGGA
GGCGGAAGGGCAGGGCCGTGGGCCGCCTGCCCCTCCCCACTCGCGGCCTAAGTGGGTCCCACTTCCCACCTCCCACCTCCC
ACATACAGCTGCGCTCGGCCGGCGCCCAGGTGATACAAGACTTAGTGTGCGACGCAGGCGCTGTGAGCTCCCTGGATCTGG
CGGATAACGGTGAGGCTGCAGGAGAGCCCATCCTCGCATCATCCACTCGATTCCCAATCCCCACCCTACCCTTGCAACTTCG
CCTCGTGCGTGACCCGAGTCACCCCCAGGCTTCGGCTCAGACATGGTGACTCTGGTGCTGGCCATCGGGAGAAGCCGGTCC
CTGAGACATGTGGCGCTTGGAAGGAACTTCAACGTCCGGTGCAAGTGAGCCCCCACCCTACTCCTGGGCCTCCCAGACAAC
ACCCCACCACCCCTGTCCCCCACAACTGCGGCCCCTGCCCACAGGGAGACCCTGGACGACGTCCTGCACCGGATTGTCCAG
CTCATGCAGGACGACGATTGTGTGAGTTCACGGGACCTTGCAGGGCCTCGGGCAATTAGACCACTTTGGTCCTCCTTTCTCT
TGTTCCCTCAGACCCTGTGACCTGCCCTCACTGACCCCTGACTCCAGCCATCAATGGCTTTCTCTTAACCCCAGCCTCTTCAG
TCTCTGTCGGTGGCTGAGTCTCGGCTGAAGCTGGGTGCCAGCGTCCTACTCCGGGCCCTAGCCACCAATCCTAACCTGACCG
CGCTGGATATCAGCGGCAACGCCATGGGGGACGCGGGCGCCAAGTTGCTGGCCAAGGCGCTGCGGGTCAACTCGAGGCTC
CGGTGGGCGGGGTCAGAGGGGTGGGACCAGCGGGCAGGGGGCGCGGTGGAGAGGAGGGCACCGGGCTAAGGGGAGGGAC
TGAATGAGGCGGAGCAAATGGAGCAGGCTGACGAGGCGAATGGACTAGGCCGAGGGTTGGGTGGGGCGTTGGGAAGCTCC
GTCCCCGACTGAAGCCAGGCCCGGCCCAGGTCTGTGGTCTGGGACCGGAACCACACATCTGCTTTGGGTCTGCTGGACGTG
GCGCAGGCGCTGGAGCAGAACCACAGCCTGAAGGCCATGCCTCTGCCACTGAACGACGTGGCCCAGGCGCAGCGCAGCCG
CCCGGAACTGACAGCACGTGCAGTCCATCAGGTGGGCGTCCCCCTCTTCCCTTGCCCTTCTCTGCACGGTAACTCCGTCCCT
CG
6 chr5: 551112-551430
CGGATGCCGGCCTCCTGCCTCGGCCACCGCCCTCCTCTTCAAGGATGCTGTTTTGCCTCTGGTCGCTCTATCTTATCTTTTTTC
TCCGCACTCCTGGACGTCTCTCGGACCTTTCGCTCTGACAGTGGCTTGAGCCTCGGCCGCCACTTCCGCCCCTGCTACTTCGG
GGCCACGTCTCGGTCCGACGCTGAGGGGTGCTGCCAACCGTGTCTTCTCTGTGAACGTCCTCACGATGAACGCCCCCGTTCC
ACTTCCCACCCTGGAGCTGCTGCTTCCGTGACTCACATTCCGGCCATTTCCGTGGTGTGAGGTACGGGCG
7 chr19: 6426223-6426998
CGTTACATTGCGATTCATTCAGGAACCCACGAGACCTCTCCCGCAGCGTCCCCGCTGCTCACCGGGCTGCAGACGAGTGCGT
CGCGGTACCCCCCGAAGAGCAGGGCCTGGGCTTTGAGGAAGAGACGGGACACCCCTTCCCCGGGGGCCAGGGCGACCTTC
CTGAGCCGGAGCCTCAGCAGGGACACCTGAAGCACAAGGGAGTGGCCCTGAGTGGGCAGGTGTGGGCCCCTCGCCTCCCG
GGGCAGGAAGGTGGGAGGGGCGACACTGACCACGTCTGGAGGCAGCGCCTGCACGTCGTTAAAGGTCGTCTCCAAGGTAT
TGGCGTCCACGTTCAGCACCACGACGTCCTCCAGGGCTTTTTCTCGTACTCTCTGCGGAAAAGCGGGGTCGGCCGCTCAGAG
CCCGGGAGTCCTTGGCAGAGGAGGGCATCTGGGGATGTAGAGGAAGGGGGAACCCTCACGTCCCCGTCGTCTGGGTAGAT
CCACGCTCACCTCGGCGAGACTGGCGTGCACTCCAATGAGGTAGGGCATGGGCGCGCTGCGGACCGAGGGGACGGGGTCA
TGCAGGCACCGCCCCCAGGGCCTGCCCACAGGCCCTGCCCCTCCCAGCTGCCCTCGCTCACCAGCAGTAGTCCAGCAGGTG
TGGGGGCAGCGTGGGGATCAGCACGTGCTCCCAGCGCATGGGGTACAGGAGCGCGCAGGACGCGTGGACGCACGAGGTCA
GCTGGGGAGCGATGGCGGGGCGTGGAGTCAGGGCCTGGACCCTCTAGCGCG
8 chr3: 32836146-32836433
CGTGAAATCGCCGTTCGTGGGCCCACGGCTGCACCCCGAGATAGAAGCGGAATAAAGGGACGACGTTACCTTCACCAGCCT
GAGTCTCTTGGGCCGCGCCCGCCTGCCCCGACTCCAAGCCCTACAGGAACCGGTTTCCCTGGTTAGGAGGTATCCACCCGGC
TCAACCAGGGTCTCGACTTCCTTGGAAAAGACCAAGGCAGCCGTACGCTGCTGGAACGCCGGAGTGCAAAGGCTTTTAGGG
CTACATTGCAACCCTCGGGCTCGATTTCGCCCTTTGGGCTGTCG
9 chrX: 129301145-129301626
CGTTGCCGCCTTTTTCGTACTGAGCGCGCAGGGCACTACGCCCACCACTCCCCTTGACCCACTCCTCAAGCCCTGATACCGG
GGCGAGGCCCCTTCCTTTTCTAGTCCTTCTACGCGCCAGGTGGGTGGGTTTCGCGGTGACGAGGCGACACCTCTTTCCCCTC
TGGCGAGCACGTTAACTGGGGAAGCTACCAAATTCTCACGCCTTAGCTGCCCTGCCTCCACCTCCCGCAAGTCCGGTTGCCC
CTCTAGGCTTCTGCGCCTGCAGTGGTTCCGACCCTCGCCTTTAGAACTCTGCCGCTTCCGCCTCTTATTTCGCGCCTTTGTTTT
TAGTTTTGGAAGGGGCGGAGTTGAAATGGCTGCGCCGCGGTTGCCGAAGCAACGCTTTGGTTACTTCCGTTGGTTTCAATGC
TTCCGGGTTGGCGCTGCAGTGGCGTTTCCGACTGTGGGAGCCTCAGCTTCCCAGTCGTCCGATGAGCCCG
10 chr9: 124027565-124030907
CGAGGCCCAGGCGAGTGGCCAAGACGCGGCAGGACAGGAGGATGCGCCTGGCTGCTCTCGGCTGCTGGACGCGGCCTGCA
GTAGGGAGAGCGCAGCCCGGGGTAATCCCACTCAGGGCTCCGGGTGCCCAGTTCTCCCACTTCCCATCCTGGCGGCCTAAA
GGCAAGGCCTAGACCCTGGAGAGGCCAAAGTCGGGCCAAAACAACAGCGAAGCTGGGACTGGAACAGGCGCTGGGGGCC
TGCGCATGACCCCAGGCTCAGAATGCGACACTCCAGTGACGCCGGGGAATGAGTTGAGATGGAGCCGGAGACAGAGGTTG
GGGTTCAGGACGAGGCGCTTGGGTATGGGATCGGATCTGAGACGGTTGCGGCGCTGGTGCTTTGGGCTGGACCAGACTCGA
ACCCAGAGCGGAGCGAGGGTCAGAGCCAGGGCAAGAGTCCGAAGCCCAGCTCAGCGTCCAGCCCGCGAGAGACCCCACCA
TGAATGACGTCCGAGTAGGGTTGGAACTCGAAGCCCGAACGCACTGGAAATGCAGCCGGACGACGGCTGGGATCCGAGTG
GGTCCGGGCCAGAAATGGGGACCTCAGAGCTCCACCGAGGCGCTCGAGGTCAGAACTGAAGCCTGAGACTTAGGCAGGAC
CCGAGACAGAGCCAGAGACGATACCGAAACCCAATGGACCGCGAGGACCGAAGGCAGATCCGGGGCGCAAACCTGTGCA
GGCACTGGCGCGCTGCCTGGAGCTCTGGGTTCGCGCCGCTGAGCGCCGGCAGGTTGGACCAGCGCTGCGCGCCGGAAGTGC
ACTCGGGACGCCGGGGTTCTGGAGGAGAGACCACACGCCGCAAAAGTGGCCTCCGAATGCGCCCGGGGCCTGCCCTCGGC
GACACTGCTGGCTCGGCGGCCGCAGCCTACCTGAAGTAGTCCATCTTGCAGAAGATCTCCTTGTTCTTGATGTAGCAGCTGT
TCTGCTGCCTCAGCGACGTGCGACACACGGAGCACTCGAGGCACCGCACGTGCCAGATGAGGTTGTTGACCTGGGGACGGG
GCGGGGACGGAGGTCGGCTCAGCGCGGGCCTCTTTCACTCCGGGCCCCAGCCTTCCCGGTCTCATTTACAGTCAGAGGCGC
TGAAGCTCAGAAGGTGCATGCGATTCCCCTATTTTTCTCCCGTAATACTGAGACTCAGGGAAATGGAAATAAACTCTTCTTA
TTTTTCAGACGGAACCCGGGGGCTCAGGCAGACCCTAAGTCTTGCCCAAAGCTTCGCAGTCGGGCAGCAGTGGAGCCAGGA
TTTGGAAGTAAGAGGACCTGCGGTGCTTCCCTGCAGTCTCCTGCGCTGCGTCCCACGCCCCGACAACACGCACGCAACACC
TACCCCTGTCTCACCTTGAGCAGATATCGGTCCAGGATCTCGAGGCCGCAGCTGGAGCAGATGTTCTTGCCTGCAGACGGC
ACGGAGGAGGCGGCAGAGGGCGGTGAGCAGACAGATGGCGTGCTGGGCGTACATGGGGAGGCCTGACCCTCGTCCTTGTC
CAGAGCTCCTGCCAGGGCCTCGGCGTCAGACTGAGCCTGCGGCGGGGGAGAGAAGGAGAGGAGCGCTGATCCGGGCACCC
AGAGTTGGGGCTGCCTTGGAGACAAATCTGGGGCCCCCGAGCACCAATTGACAACGAAATCTCCCCAGGACAATGCGCCGT
AGACTGAAGGACAGGGATGGAAGGGCCTATTGGAGAGGAAGGGACGGCCCCAACTTTTAACACGCGCATTCTGGGTGCTG
GGAGCGGTTAAAGCCAACATTCCCTGCCGGAAAACCGAGGCTCAGAGAGGGGCGGCGCCCGCCGGGAGTCCCCCAGAGCG
TGAGCAGCAGTTCGTGGCCGGAAAACCTGGCCAGGTCCCCAGGGCCGGGCCGCTGGAGGACTGGGCACCGCAGGCAGGGC
CAAACGGACTCACCATGGCGGGCGGCGCGGTCCCTTCAAGACAGCGGGTGGTCGCTTTGCAGCCGGACCCTGGCTGGGCCA
TCACCTGGGGGAGGGGGGGAGGGAACGCAGGCGGCGGCGGCTGCTGAACTGGCTCCGCACAGCCTGAGCCCAGCGCCTCC
CCGCGCCTGTTATATAAACCGGCGCCGAACAATGAGTCCTAACTTTGTAGTGGGCATTTAAAGCCCTTCCCCACAAAAGCG
GTGTCTCTCTAATGAAGCAATTTGAATTTGGATTGGATTTTTTCCCTCTCTCTCCCCCTCTCCCTGCACTTAACCCGTGGCTCT
TGAAGTAATCGCTTAGTTCCCTTGCAATCCAAGCCTCTGAGGGGGGAAGAAAAACACGCGCACACACACACAAACTACCTG
CAATTAGAAATTGTCGTGAATGCCCAAGATAAGAGGTAGCCAGAGTGTCAATTCCAACTGTCAATCAGTGAGATCCATCAG
GCCGCCCAAAGAATTGCAAATTTGATTTTTTAATGGTAGTAATTAAAAATCGAATTTTTTCTCTCTCCACCCGCCCCCCCACC
CCACTCCCCTTCCTCGCTCCCTTCTCCTCTCGGCACAAAATGCAAAAAGGAGAAAAGAGAGAAAGAAAGAAAATAAAAAA
GAGATGGGTGAGGGGCGGAGGGTGGTTGAAGAAAAGAAAACCCCGAGCGCGGTGAGCACCCGCCCGCCCGCCCGCCTGAC
ACGCGCGGCGAAATTGAAGCAGCTATATTGACACGGATTCAGGCGCCTTTCGAGGGCCAGTCGGAGGGAAACCCGGAGCA
AAAAGAGAGCGAGATCGGGGCGAAACGGGACCAAAAAGAGAAAAGAGAGGCGCTCAAGGGGAGGAAAAAGCACCCTCC
AGCCCCTCGGCGCGCCGGGTACCGGCCCCGCGTCGGGATTCTCAGCGCTGCGCCGGCACAACCCCCGGCGCATCGGCGCTA
TCAGCGCCTATTCAGACGGACAATACCGGCCTCGCCTCCTCTTGATTCGCCTGTGTCTTTACTAAATCGCTTTTTGAGAAATT
CCTCCAACATTTGCATAATGTGTTCCCGCTTTCCGCGACCCCCCCCCCCCGCTCGCGCGCGCGCTCCCACACTCACAAGCAC
ACACTCACAGGCGCACCCTCGTACCCCTCCTTCCTGCAACCGCCCGCCACCCTGCTCGGCCCGGCTGGGTCCCGGACCCTGC
CCGACCCCGGCCCGGGCCGCTCACCCAGTCGTTCGCCGGCTCACCTGGTCGGTGGCGGGGCCGCCCTCGGCCGGCAGCCGG
CAGCCCTCGGGCAACGCCGGGGCGGCGTTCTCATGCTTCCAGTACATGGGCCGGGGAACCTCGGGCTCAGCGGGCGCGCAG
CGCGGAGAGCTGCGCCGAGGGGGACCACAGCCG
11 chr10: 129424401-129427356
CGCCTCTTTCGCACAGGTGCCCGACTCGGAGTCCGGGAGGCGGGCCCTCTTTCCAGGTACCCAGCCCAGCGTCAGCCTGGG
TCCATCCTCCGCCACCCCACAGCGCGGGGAGCAGGGGAGGCCGAAAGCCGGGCAGCTTGGGGGGCCGGGATAACCCGGGC
TGCAGCAACCCCGGGCGGCAGCAGGACTGGAGAGCTGGTGTCGGACGCACTGCAGAGACGTCCCTGTTTTCGGCGCGTCCT
CCGACACCCAGCAGAGAGCTTGGCGCTCTCCGCTCTGCGCGCCCGAGGTCACAGCGCGGAGAAACCTGGCGGGGCCCCGG
ACTCCCCGGCTTGGGAAAAGCGATGACTGCCCTGAACTGCTGGGGCGTTCGAAATTTCCAGGGTCCCGACCCTCCGTGGGG
TACGCGCGACTTCGGCGCAGATGTCAGTCCGCTGCCTTCCGGGTTGAGGGAGCGAGGACTCCAGACGACCCCAGGGCCGCT
GTCCAGGCCCAGCCCCGCGTTCTCCACCTCGCCACCTCGCTCTGCGGCTCCAGCCTGGAGATCTACGGACTTCATTGCGATC
TCTTGCGCAGTGTAGTCGCCCTCTATCCCTAGCCCCGAGTCCCGGGTAACCCAGGGGCCCCGGCGAGGCGAGTTTCTTTGGG
GCGAGATGTCCCGAGCCGAGCAAAGGGGCCGCGCATTGGACGGGGCTCCCAAGCCACAGCCCTGGGGGCCTTGAGGAGTC
GTGGTACGGTCCAGGGCAGTCGGGGCTGCGGGGCCCGTGAAGCCAGACGAGGCTACGGACTTAGGGTCCCTGCAACCAAG
ACCGCGAGCGACTGTGGACTCGGGACGGATCGCACAAAGGAGGAAGGGGGAATCAGAGGGGCGTCCGGTCCAGGACGTGT
CAGGCCTGGGCGGGGCGGAAAACTGACACCAGGGCGAGGACAAGGATTTCGCTCCTCCAGGGCTGCCAGGGGAGGGGCTC
GCCACCGTCACTCTCCATGGGCTCTGGCCCCGACGGTGTTTAAAGGGCGGAGGGCTGGGCCGGGCTGGTCGCACCCGGGCG
CTGCTGGCGGCCAAGCTGGATGGGTCGCCAGTGAGTTTCGGTGCGGCACCGCTGGCCCAGGCCCGGGCGCGGCTGGACATG
GCCACCTACTGCGACGACCTGGGCCCCTCCTCGGCCCCGCCCGGCCAGGCCCAGGCCACCGCGCACCCCCCGGGCTATGAG
CCAGGGGATCTGGGCGCGGTGGGCGGGGGCCCCCTCCTGTGGGTGAACGCGCCAGCGCTCAGCCCCAAGTCCTACGCTTCG
GGTCCCGGGCCTGCGCCGCCCTACGCGGCCCCGAGCTACGGGGCTCCCGGCCCGCTCCTCGGCGCCCCGGGCGGCCTGGCG
GGCGCCGACCTCGCCTGGCTGAGCCTCTCCGGCCAGCAGGAGCTGCTGAGGCTGGTGCGGCCGCCCTACTCCTACTCGGCG
CTCATCGCCATGGCCATCCAGAGCGCGCCGCTGCGGAAGCTGACGCTCAGCCAGATCTACCAGTACGTGGCTGGTAACTTC
CCTTTCTACAAGCGCAGCAAGGCGGGCTGGCAGAACTCCATCCGCCACAACCTGTCGCTCAACGACTGCTTCAAGAAGGTG
CCCCGCGACGAGGACGACCCAGGTAACAGCGGCGCGCCGGCTCGCTCCGTCCAGGACTCCCCATCTTCCCGCCGGGCCGCG
GGCACTCCGGGGGTGGGAGCACCTTAGACAACCTAATTTCTCCCTGCGCGGTTGGGGATACCCGGGGAGGAGGGAGAAGG
GGAAGAAGTGCTTGGGCATGGACAGGTTCTCAGACAGCGGCTGAGCCCACCACAGGCCCGCGTTTACAGTGCGGCGGTCCC
GGGGCTCTCAATCTCTGTGCGCTCCTTCGGAGGAAGAAGCGGGAGAGCCAGCAAGCTGGGTGACCTTGGGAGTGACTCAAC
TTCCCTGGGCCTCAGTGCCCTACCTGGTAAAATGGGGAAGACAGCGCCTACCCCAAGGATGGGCAAAACAAATAGGATGCC
CGTTTGGAAAGCTCTGGAGCCTACCTGGGGGCCTGGCGTGGTCTGCGCTGTCCTTGGCAGAGCGGCTGGCGGAGTCCCACC
ATTAGCATCTCGCAGGCCTTCCTTCACCTTAACGGGGCTCGGCCTTTCCCAGGGCTCCCTTCCTCCTCCTTTCTGGATTGTCA
CTTTCTTCCCAGTCCTGCTGTGGAGGCCCATCGGGTACAGAGTGCCCGTCAAGGGTGGAATGGGGGCGAGGACTCAGACCC
TCTGTAAGGGCCAGCAGGGGATCTTTCTGAAGCTGACCTCCTCGCTGGCTCACACCACGGCCCGTCAAAGCTCGAACCTTCT
GAACTGGGCTGTTTCTTCTTCTGCAGGTAAAGGCAATTACTGGACCCTGGACCCCAACTGCGAGAAGATGTTTGACAACGG
GAACTTCCGAAGGAAGAGGAAGAGGAGAGCTGAAGCCAGCGCGGCCGTGCGCTCGGGAGCCAGGAGCGTGGGAGGGGCC
GAGGCGCCAGCGCTGGAGCCCCCGAGCGCGGCTTGCCTGGACCTGCAGGCCTCGCCCTCTCCATCCGCACCCGAGGCCGCC
ACCTGCTTCTCCGGTTTCGCTTCTGCTATGAGCGCTCTGGCTGGCGGCCTTGGCACCTTCCCCGGGGGCCTGGCGGGCGACT
TTTCTTTCGGGAGGCGGCCACCGACAGTCGCCACCCACGCTCCCCAGACCCTTAACCCCTCCCCTGGCTTCGCCCCTGGCCA
CCAGACCGCGGCCGCCGGCTTCCGCCTCAGTCACCTCCTCTACAGCCGGGAAGGGACCGAAGTTTGAAGGGAGGCTGGAGG
CTAGCCGGGTGCGGGTCCAGAGGTGCTGAGCTCAGGCCTCCGGTTTCCCCTGGTGACAGCCCCACGTGTTGGCGTTGAAGG
CCTTGGTGCCCTGGGAGGAAGCGCAGAGCCGGGGGCGGCTCG
12 chr1: 3147370-3148160
CGGGGTGCGGGGAGGTTGAGAGCGCGGCGGCCGCTGCCAGCAATCGAGGAGCCAGCGGCGCGTGTGCTGAGGGCCCAGCT
AGCAAAATAAAGAGGGTTTTCAGCGGAGCGGCGGCTCAGGCGAGGCTGGGGGAGCCGGGGAGCGCGTCCCGCCATTTTTC
CAGCCGAGGTCAGCAGCGGGTGAAGGGGCGGAGTCCGTGGTGTTCCCAACTGCCCAGGCGGGCGGTGAGATGGGGCAGGC
GGTTCCGCAACGCGGCTCAGGCCCCAGCGCCCTGCCTGCAGGAGGCCGCTTCTTTCTCTGTTGCCATCGGGCGCCGGTTTCC
TCTTCCGGGGATGCAGGGCAGCTGGGGGGGCTCTGCCTCTGTGCGGCTCTTCCGTGGTTCCACGCTGGGCTCCGGGGTGACC
ACAGTCCTCAGCAGCCCACACCCTCAGCCCGAAGGGGGCCCAGGCCCCGTGCCCAGAGGATCCAGAGAGACAGCCGGGAG
CCCAGCAGCTGTGGCAGCTGCCCCCACCACCACCCCGGGGCTGGCCAGGTCCCACCTCCTGACGCCACACGGTATGACTGG
GCCCTCCTGGTCAGTGCGTCGCATGTAACGCCTTCAACTATTGGGGCCGGTGTTGGAGCATCCGTCTCTGCGTCCCCACCTG
ATGACAAGCTCCGGGGAGCAGGAGTGCTGGCCAAGCCCACGCACGGACCAAGCCCACGTCAGACTCCGCCAGAAAGGCAA
GAAGGGCCTCCGCATGGCGTGACGGATGGGTTTATGGGGGCATTCCATCCCACCCGGACGCTCG
13 chrX: 128941527-128945710
CGGAGCTTGAGCGCCAGAACCACGCACTGAGGTCGCCCAGGGTGGGGAAGTGGGGGGCCGGGGGAGGGGCGGGGCATCC
GGCGGGGAGGTCTCCCCCACCGCATGCAAGACCCTTTGCCCCCCCACAACATACACTGAGGCTAAAACGGGGATGAGAGTC
ACACAGAGCAGGAGCGAAATCTTTCGCTCTCCGACCCGAACGGCGCGTTGCTCGACCCCCCCCTTCCTGGAGCCAGGGGCT
GGCCGAGGAAGGTGGCTCCGTTCTTCCCCCGTGGCCAGTGCGGGGTCTGCGGCTGCCCGGCGAGGTTCCGGCTTCCCCGGG
ACTGGCAGGGGGAGCCCCGAGAACTGCAGGCCCGCCCCCCCTTCCTCCTCCTCCTTCCCCTCCTCCCCCACGTGTCCTCCCT
GAGCCCAGCAGCCTGATCCCAGCACCCCCCGCCACCTCCCTCCTCCCGCTTCGCCCGCTTCCCCGGCGGCGTGGAGAAGAG
CAAAGTTGCGACAGCGGCCGGGGGCTCTGCCCAGGTAAAGGGGGCGCCGTGAGGCGGGGGTGGTGGGGGAACGGTGCGGC
CCTGCGGCGGGGGGGGGGGGAGGATAAAGAAACTTGCTTCCCGCGGCCCCCACCCCGCGTGGGTTCCGATCTTGCGTGGAA
CGAGCCCGCGTGGCGAGTCGCGGCAACACTGCCGGGACCGAGGCCGGCACCTGGACCCGGACCCGGACCGCGCGCTTGGG
CCGGCTCGGCGGGCGCAGGCAGCGGGCCAGGCCAGCACCCAGGCAAGGGCGGGGTGGGTGGCCGTACACCGGACCGAATC
GGGGACTCCGGTGGCGTGGGCGCGGGCCGAACCCCGACCGGGCCGAGTTGGGTGGCGAAGTAGGACGTGGATTCGGGGGC
GGGGCGCGGCGGGAGGGGGAAACCAGGCCTGGCCAGACTGGCCTGGGCGTGGGGGCGGGGGGCGAGTTCGGGGCACTTG
CTGGGCTCTGGACCGAGCGGTAGGGCGGGGCGGCGCTAGGGGCTGGGCCGGACCCGCAGGGAGAGGGCGAGGGGAGGGA
GAGCCGGGGGTGGAGTAGGGGCGTGGAGTTGTGAGCGGGGTCTGGCCTCGGCCGGGGGCGAAGGTAGATCGGCGGGGCCG
CGAGCGGAGGGAGGGAGGCCCGCGGCGGCGCGGCGGCAGCGAAGGCCAGCTTCCGCGGAGTTTGTGCCCGGGCTTCCCGG
GCTCTGGCCGCCTCACGCGCACAAATGGGGCTAGGGGACTGAGTGGTAAGCAACTCCGAGTGTTAGACGGTGATCGGGCG
GCGATTCCGGGAAAAGCGAGGAAAGACACAGTCTGCGATTGTGCCGCACCCCCCACCCACCTCTTAGCATCTGGATTCTGC
TCTCGTAGTGGGGGCCGCGGACCCTCCCCGCCACAGTCCTTTTACTCTCCAGCACTCCCACCGCCTTCCCCCTTCTTCAGCCA
TCTGACTCTCCTAGGGGTATGTGTTTTTGGGGGTGGACGGCGACGGGGGTGGGGGATACTTTCCTTCCCGTGGCGGCAACCT
AGTTCGTATCTTATTCCTACAAGGTAGCGTCGCTTCGAGTCGCATGGACATTGGGGTTTAGATAATTTACCCCCCACCCAAG
CGATTTAATGGCTTTTAAGCAAAAGGCGAATACCTGGATTTCCATGGGAGGGATGAGGTTTGAAGATTGGGATTCACCCCT
AGCTCACTTCCACTACCAACCCCTCTTTCTTGTGAGCCACTTCCACGGGTATTCAAATCTGGGGGGGAGGGGGGACGGGAG
TATTAATATGCTTCTCTATTTCTACACAGGGTCGGCGTGGCGAAGGACGGCTAGCCTTGGAGGGAAAGTAGCCACCAGTCC
AACTCGGGTCGCCCCCACCATTATTTCGGTGAGGGGGCAACGCTAAGAAGGGGATACTGGTGGGTAGGGGCCCAGTATAA
GGGAGGGGGCTGGGTAGTCGTCCCCAGAAGGACTCCGTGTCTTGGCGGGGAGAGAGAGAGGCGAGCCCCGGCTTCAGGGG
AGGGGTTCCGGGGAGGGAGATGGGGGGGTGGCGACGACGGTGGGGGGGGGAGCGGGAAGAAGGGGGAGTCACGCCCTGC
CTGCAAGGGGAGCCTCCCTCAGGCCCCTGGGAGAAGGGGACTGAATGCGTCTGTTCTCCGCTGCCCGGCAGAGGAGATCTG
GGGTCTCGAGATTTGCACACTGGATCGGCACCAGTGGCCTGAGAAAGTCAGGTCGGGGCATAAGGTGGAGAACGTTGGAA
GAAAGAGAGAGAACCCTTTGGCCGCGGCTCACTGACCCTCTCCCTTCCTCAACCTCCCCCTTCGAGGGGCGTCGAGGTGGG
GGGGGACGTGGGGCTCCTGCAGCCCCGCAGATTGAGCGCCTCCGCCTCTCCCAGGAGTCTAGCTTTTTGCCCGGCTTGCTGC
AGCCACTGCTGCCTCCCTCTCCGGAATCCGCTCGGTCCTCTCAGCGATCCCCTCCTCCTCTCCTCCCCTCCCAGTGTCACCGG
GGCTGCTTGGCTCTGCCCCCTTCGCCGCCGCTTGCTCCCTGCTTTCGTCCCTCCCTCTCTTGCTCGCTCCCTCCCTTCGCCTCC
TCCTCCCCATTCCCCCCCACCCGCGTAGGCCGCATTCTGGGAGTTGTAGTCCGGTGGAGCGGGGGTTGGCGCCGCCCGCGG
ACCCAGCCGGACTCCACTTCCCGTCGAGCCCTGCGACCGGCACCCACTCCACCAGGCTTCGCTCGCACACAGACACACACA
CACACCGCTCTCCCCCTCACTCTTTCGCTCGCCGCGGCTGCTGCCAGTGTGTGGCTCTGTCTCTCCTCCGCTTTGCTGAGCCC
TCCCTTCTTCCTCTCAGTTCCTAGAGTCCGACCGCCGCCGCCGCCGAGAGAGAGGAGAAGGAGGTCGGTGGCGATAAGGGG
CGGAGGGGGGCATCAGATCGGGGCAGTATTAGGCGGGTGGCGGTTAAGAGGGAGTAAGAGGGAGCCCGGGTGGCGGCCT
GAGCCTCCCCGCGGAGCCGACCCGGGAACAGGTGCGTCTTTTTTCTCTCTCCCCCAATCCCTCCACCCCTTTTGACTCCCCGG
CTTTTTCGTATCCCCCCACCCGCTTCTTTCTATCTTAAGCCTTTCTCCAACCCTGTTCCTTCTTTTCCCTGCGGCCGCTCGGCTT
GCCCCCCGGGGGCGCCGTCCTCTTCGTGTCCCCGGACGTAGCGCTCCCTGGAGCTGGGTATGTCTTCGCCCCTCCCTTCGGG
ACAGCTCCCTTCCACTTCCCACCTTCGTACCCCATCCCCTGACTTGCCCTACCCCCTTCTCGACCCCCTTCTCTCTTAGCCAG
GTGAGACTCGTTCGCCACGATCCAAGAGAGTCTTCTTCCCGGCTGGGCGGGGGTCTCCATGGAAACGGGGGTCGGTTGATC
CTGGGGGGGAAACCATAGGAATTACCCCACTCCTCGGAAAGGGAGGTGGAGGCTATGGTCTTGTTAACTACCTGACTCCAA
CCCCCGATCCAAGGATCCGACGATCCGAAGGGAGGCTGGCTAGCAGGGCAGGGCGAGAAAAGGCTTTTTTTTGGGGGGGG
GGGGTGTCCGTGATGAAGTGTTGAAGAGACCGGGAGAAAATGCCCTCCAACCTCAGAGGGGAGGTGCGACGGGTGCTCTT
CAGGGCTGCTTTCTTCCCGGCGCTGGGGTGCGGGAATCGTAGGGTAAGGGGTACCCCTAGGTGGCGGCGGGGCCTGGGTTC
GGGAGTGGGAGTGCTGTGATGGGGGCAGGGGCAATTATCCGAGTCCCTGGAAAAAAGGCGGGGGATGACTTCGGAGCGCC
CTGGCTCCGCTGCCCTCGCTCTAGGGGGGGGGCCGCCGGGCCGGGCGCGGCGGGGAGGGGGCTTAAATTGAAGGAAGAT
GAGATCGGGGGCAATTCATACTAGCACCGGCAAGCCAGAGGGGAGATCCGGCGTGCAAGCCCTCCACCCGCTTCGCGCCA
GGCCTGGAGGCAAGGGGTCGGCGAGCGAAGCCGAGGGCTGGAAGGAGGGAGGCTCCGGTTACGGGGCCGGGTTTCGGGAT
CAGGGGCAATCCCCCGGCCCTTGGAGAAGGAGGGAGAGGGATGGCCGCGAACCCCAGGGCTCCCCCACCCCGGGTCCTAA
GGGGCGTGAGGGACGGACAGCAGGCCCAGCGGAGCTGGATTCGAGCGGATCGCGGCCG
14 chr17: 7584523-7585036
CGAGTTGGTGCGGGACGTGCCGCACGGCGTGCTTCTGCTGGACACCTTCCACAGGCTTGCCTCCCGCGAGGTGCGGCTGCC
CCGCGGCTTCCTCGGCTTCCCGTCCCGCGTGCTTTCCTGGAGTCCCTCCTTCCGGAGGCCCTCCTGCCTCCACGTGTGCCCTT
CTCCATGTCCAGCATTCGGGCGCCTCTTGTCTTTCTTCTGTTCCCTGGCTTCGGCGTCCCCGGGAGTGTGACTCCCGCAGCGG
GGTGCAGCTTTCCTCTGGGATGAGTGACCGGAGGGAACCCGCCTTCCCGGGCACGTCGCCAGCCTCTTCCTCTTCTTCCCTA
GGCTATCAAGCGGACTTATGACAAGAAGGCGGTGGATCTCTACATGCTGTTCAATAGCGAGCTGGCCCTGGTGAACCGTGA
ACGGAACAAGAAATGGCCAGACCTGGAGCCCTACGTGGCCCAGTATTCCGGAAAGGCGCGCTGGGTGCACATCCTCCGGC
GTCGCATCGACAGAGTCATGACCG
15 chr1: 27548507-27550603
CGAGGAGGCTGACCCGGAGCTGGAGCCCGCGTCGGGGGGAGAGCAGGAGCCGCGGCCCCAGCAAGCCCAGGTAGGCGGG
AGTGGCCCGTGGCTGCTCTCAACATCCGGAGCGGACTCCGGGCGGGGAGCGCTCCTGCCCAGGGCTGCGAGCCGCCCGCGA
CCCAGGGCGCTCGGGGCAGGGGTGGGGAAAGAAGGGGCGCCCCGTCACTTGCCCCCTCTGCAGACCAAGGCCGCGTCCCA
GATCCTGGAGAATGGGGAGGAGGCCCCGGGGCCCGACCCCTCTCTCGACCGCATGCTCAGCAGCAGCTCCTCGGTGTCCAG
CCTTAACTCCTCCACGGTGAGGCGGGAGGGAGGGGACCCGGGCGGCCGGGGGGTGGACCCGTTCCGATGCGTAGCCCCTG
CCTGCCCCTCCCTCGCCGCGGGACCCACCGCTGCAGCCCCCCAGCCTGCCACCTATGACCCGGGTCTGAAGCCTCCGCGCTG
CCCGCGGCCCCGACGTGAGCCCTGCGAGCGGCCCTGACTCCCACCCACTCCCGTCCGCAGCTGAGCGGCAGCCAGATGAGC
CTGTCAGGCGACGCGGAGGCGGTGCAGGTCCGCGGCTCCGTGCACTTCGCGCTGCACTACGAGCCGGGCGCCGCCGAGCTG
CGCGTGCACGTGATCCAGTGCCAGGGCCTGGCCGCCGCCCGGCGCCGCCGCTCGGACCCGTGAGTGCCCCGCCGGCCAAGC
GGGGCGCGGCTGTCACAGCCCAGCCCACCATTCACAGGGTCTCGGCCTCCTCGTCCTCATCTTCAAAATGGGAACAACAGC
GTTATTGGGAGGCGTGCGATTAAGCGAGACAATCCCTGTAAAGCGCTTAGCACGAGGCCTGGCACGTGTTCGGGATGGTGG
CTGGGGGAGCCCACAGGCAGGGGAGAAGGCTCTGGGAGGGCCCCTCCTCACCTCGGGTTCTCACCTCCCCAGCTACGTCAA
AAGCTACCTCCTCCCGGATAAGCAGAGCAAGCGCAAGACGGCGGTGAAGAAACGGAATCTGAATCCGGTTTTCAACGAGA
CTCTCCGGGTGAGGCTGTGACCACGATGCGGTTCCCCGTTAATGAACTGGACGCCCCCTTCCTGCGGGGCTAGGTGGCAAG
GGCAGCCAGTAACGTCATTGCCCGGAGGATCGGCGGAGGGGGCCCATTAACTCGTTATCCAGTGTTGTCAGCCTCGTGGTG
GGCGTGGTGATAGTGCAGGTCCCCATTAATGCCCTTAGGGGCTCCCCAGAATTCCATCATGGTAGGAACGCGGTAGGACCT
GCCTCAGCCAACTCGCGCAGCATCTACGCGGGCCACCAGCAGTGCTCCACTAAAGCTCACCTCCTGTCCTCAGTACTCCGTC
CCGCAGGCCGAGCTTCAGGGCCGCGTGCTGAGCCTGTCTGTGTGGCACCGCGAAAGCCTGGGTCGCAACATCTTTCTGGGC
GAAGTTGAAGTGCCCCTGGACACGTGGGACTGGGGCTCTGAGCCCACCTGGCTCCCCCTGCAGCCCCGGGTGAGGCAGCCA
GGCCGCGTGGGGAGACCTGCGGCCCGGGTCTCCTGCATTTACCCCACCAGGCTCTCCCGCAGCCCCCTCACACCCCGCCTTC
GACAGAACCTCCCCTCAACCTCTTAACCTCATGGCCCCAGGCGAAGCCCGGCCGGCCACGGCCCCTTCCCCGAGGGCGCTA
GGACCCCTAGGTTCTGCCCCTGCAGGCCCCGCCGTCTCTTCTAGCCGCACCCCATCCGGGTCTGCAGACCCCACCCTCCTGA
GGCCCCTTTCCATTAGCCCCTGCTCCACGATAAGCCCGCCTCTCGCAGGTCCCACCCTCTCCCGACGACCTTCCGAGCCGCG
GGTTACTCGCCCTGTCCCTCAAGTACGTCCCCGCCGGCTCCGAGGGTGAGTGACAGCCGGAGAGGCCAAGCTGGACACGCC
CTGAAAAGCGGGAGACTCCAGTCCCCGGGTTTGGGGGCGGTGGACTCCATCCGTGTGCGGGCCTGAGCCGAGCCTCTCCGC
AGGCGCAGGACTGCCCCCGAGCGGGGAGCTGCACTTCTGGGTGAAGGAGGCTCGGGACCTCCTGCCGCTGCG
16 chr19: 10304689-10307022
CGCCCCCTTCCATTCCCCGCCCGCTTCCATCACTTGGTGTCGCCCAAGCCTGTGGTCGCCCCTAGCAACGCCCTCCCTCACTT
CGCAGTCGGTTTCCCCTTTCATCATCGCCCCCCCACGTCCCGCTCTTGGTCTCTCCCGATCCCGCGTGGATCCGGTGCTTGGG
CGCCCCCGCCAACGACCCCCGCGCACCTCAGCGTTGGTGTCGCCCGGTTCCCCCCGCGCACGCGCACCGTGGTATTTCCCGC
CACGCTCCTACACCGCCCCCCCCAATACCTGGTTGGTCACGTCCCCCGGGAGGCCCCGGATCAGTATCTTGCGGCGGTTACG
GAACTGGCGCTCGGTGTGTTCCAGGCGTTTCCGGATCTCTTCTGGATCTAGAGGCGGCAGCTCTTCTTCCGGCGCCCGGCGC
TCCGCGGCATCGCCGGCTTCGACTTCGGCCCCAGACTTAGGGCTCAGCGGGGGCCGGTGAGTAACGGACACGTCCGCCGCC
ATCTTGGGAAACCCGGCGCCTTCTGGGACCAGCGAGCCGGGGCGGAGCGGCATAGAGCGGCAACGAGGGCGCGCCCGTCG
ATTGGCTGGGAGAAACCCCACCTCCTTCCCGCCCCCCTTGCCCCGTTGGCTAAGCCCCAGCCCACCTCTCAAGAGTTCAAGC
TGCGTCTGCGCGCCACAGGCCCCGCCCCCTTCCGTCGCAGCGACGCTACTCAGTGGATGTGCACCTGGGTGGGAGAGACTT
GCAGCGCGGGTGGAATCAAACCACAGATTAGGGAGTTTGAAGGCTTTATTGGTGCGGAATCTGAGGGCACAGCCAAGCCC
CCGCCAACTTTGATCCCGGATCCCAGCGTCACTCAGCTCTGGACGGTTCTTCCCCCATTGCTTCTGTCGGCTGCATAGACGT
GAGGGGCAGATAGGTGCTCTCCTCCCTAACATGGTAACTGCCGCTCCGTTGGTGCTCCCTGAAGACGTACATTAAGGCCAG
TACGATAGTCACCACGCCCAGGGTCAGTAACACCGCCACGAAGACGGGGACAAAGTGGGAGCTCCCAGCTGTGCAGAGAA
AGCGCTAAGTCAATATGCGTCCCTTCTGTCTCCAACCCCCCCGCCCCCCGGCTTACCGGTCCAGACCCGCAGCCCCGTGTTA
GCTCACCCTCAATGTCCATCACCACGACCAGGGTGTATTTGCCTCGTGAGCTGGACGCTTGGCACTGATAAGTACCATTATG
TGTTACGTTGACGAAGAACGGGATCCCCACCGGCACCTCCCGGCTGGAGCCTTCCTTCAAACACCGCAGCTCGGGGTACGG
GTTGCCCCTGGCTTGGCACTGCAGGACGTGTCTCGTTTTATCTTTCCATTTCAAGTGCTGGGGGCATGTGGCTCGGTCAATTT
TGGGACCATCTGTGGAACCACCATGTGTGATCAGACACCCAACACACCCGAGGCACAGTGGTGCAGAGGAGCGTCTAATCT
TTCCAGGGCAGGGGTGGAGGGATTAAAGGTCAGGGTGACCGACTCACACAGGACTCGCAGCTGGACGCTACTGTTCCTGTG
CAAGAACTCGCCGTCCACCTCGAGAGTGGCACTGCAGAAGAAGCTGCGTCCGTCGTCACTCTCGGTAGCATTTAGCTGAAG
TTGAGCTGGCTGCCCCGGGGCCGCGGCCGGAACTCCGTCCAGCGTGACCTGGACTCGAGCCCCAGCCATGCAACTCACGGT
CACTGTGGACCCCTCATGGGCGGTGGGCTCGCTGAGGTTCACAATGGGTCCTAGGAAGCCTAAAGGCGGGGCATTGCCCAG
GAGCTTAATGAACAGGACCTTCCTGTGGGTCAAGCCGCTCCCTCCGCCCTCCCCTTTCCTCTCGGGATATCCGGGCCACGCT
TTCGGCCGTTCAAGCCTCGCCCTCTTTCCGCGCTGTGTCCAGCTTCGGGCACTCAGGCCCAACCCACGTTGCAACTGCTATT
GGGGCAAGCCAGGCCCCACCTTTTCGGCTAGTCTCCGCCCCCTCTGCCACGCCCCCAGACTGCTGAGGCCGCGCCCCCTTCC
CACGCCTCCTCTTACTAAAGACCGTCAAGTTCTCCCGGGCCTCCCGTCTCTCGCCCCCTAGGGTCACGTTGCAGACGATCTC
CCGGGCACCCTCCTGATCCGCGCGCGCCGTGGCTGTGGCTGTGGCCGTTAGCGTGTCCCCGTGGTTCATGACTGTCGCATTC
AGCATCTGGTCCCCCAGCGCCAGGTAGACCTGGGCCTCTGAGGCTGGAAAAAGCCCGTCTAGGGTGCAGTCCACCGGCCAC
GACGTTTCCACCTCCAAGAACCGGGGGGCCACGAGGCGCGGGGGGGTCACG
17 chrX: 129132628-129134108
CGCGGCGGGCCTTCCCCATTGGCCAGCCAAACACAACCGACTGCGGCAGCCAATAGGAGCCGCTCTCCTGAACATTCAGAG
GATGGGTGCGCGTGGTGTGATAAGGGGATGTTGGCGCCTGGTACGCGCCCCCCTACATCTGGCAAGTCTGTAACAGAAACA
GTTACACCCCCTACACACGCACACGCGTGCCCGCTGGCTCCCTCCTCTCAGCTGTACAGGCCCCGGACAACCCACTGCGTGT
CTTGGCAGCGGCCTCCCAGGCTGCCTGGGGCCCGCGAGTGCACCTGCTCACTCATTAAGACAGAGCATCCCTTTATCCCAAG
GCGCGGGTCGGGTAGAGGGTGGGGGTAGAGGGGACGGGGTAGCGGGGACCGGGAAGGCGCGGCTCAGCTGCCCGCTGCTC
CCGAGGTGAAAGAGGTGCCTCCGCCCCCACCCCATCCCCGCAGAGTTGCGTTGCTCCAGGGAGGCCGAAGTGATCCAGTGA
TGTCTTAAGGTGCAGCTCTTTGTCCTCCCCAGCTGTGCTCCCTGCCCGCTGACCCCGTGCGCTCGAGCCAGTGGGGGTGGGG
AGCCCGAAACCTATAACATAAACGGGGGCAAGGAGAAAGAAGTCTATCCCGGGGTGGCGTGTGCCGCTGTCCCCACCCCG
TCTCTTTCCTCCTGAGGCTTCTTCCCTCGGGTCCGTCCCGGGAATGCAGGCAGGGGAGGGTGTGGGCCGAGGCGCGGCGGC
GGCTGGAGCAGCGCGGTAGGGTCCTTCGCCAGAGCATCCGGTCCGAGGGCGCACACAGGCAGAAGGCTCGCGGCTCGTCC
ACTCTCCTCCCTCTCTCCTCCTCTCCCTGGCTTTTGTGTTGGTGCCTCCGAGCTGCAAGGAGGGTGCGCTGGAGGAGGAGGA
GGGGGGCCCGGAGTGAGAGGCACCCCCTTCACGCGCGCGCGCGCACACGGTGCCGGCGCACGCACACACGGGCGGACACA
CACACACGCGCGCACACACACACGCACAGAGCTCGCTCGCCTCGAGCGCACGAACGTGGACGTTCTCTTTGTGTGGAGCCC
TCAAGGGGGGTTGGGGCCCCGGTTCGGTCCGGGGGAGATGGCGCAGCCCATCCTGGGCCATGGGAGCCTGCAGCCCGCCTC
GGCCGCTGGCCTGGCGTCCCTGGAGCTCGACTCGTCGCTGGACCAGTACGTGCAGATTCGCATCTTCAAAATAATCGTGATT
GGGGACTCCAACGTGGGCAAGACCTGCCTGACCTTCCGCTTCTGCGGGGGTACCTTCCCAGACAAGACTGAAGCCACCATC
GGCGTGGACTTCAGGGAGAAGACCGTGGAAATCGAGGGCGAGAAGATCAAGGTGATCCAGGGGGTCAGGTCCAGGAAGG
GTGGGACCCGGGAGGGGACCTCGCCCGAGGCATAGCTCTAGCGGTTGTCGTCGTCCAGCGTCCAGCGCGTGGCGGTTTCGC
CTCTTGCTGAGCCGAGGACCCTCG
18 chr21: 45648960-45650662
CGCACGCCCCTCCTGTCTCGCGTCCGTCCCTCCGTGCAGCCGGGGCAGGACCCTGCAGGGGCCTTGCGGGCCGGAGGTGCC
GGCTTCCTCCTCCGTCCCGTCCCCCCCGCGGTGACTCTATTCAAATCTCCCCCCTTCCCGCTGCCCCACCGGACTCAGGGGCT
GGGGTCGTAGCCCGGCGGGCAGCGGTGTTCACCTTCCGCATAAACCTGGGCTTCCTCGCGGGGCAGCCCCCGGGCCCTGTG
CTGGGTCGCAGCCTCGGGCGGTTCCCTCGGGGGGGGACGTGTCCCCGTCCGTCCTCGCCCGTCCCCGCCGCCCCCGGGTCCC
GGCCCGCCCCGTCCTCGCAGCCGCAGGTCCGCTGCGGCGCCCGCCCCCCTCTCCCCTCCCCGGGCCTTGCCCTGCTCGTCGC
TCCGCCCCCTGCCCGGCCCCTGCCCGGCCCCGCCCCGCGCCCGCCCCCTGCCCGCGCCCGCCCCGCCCCCTGCCCGGCCCCT
GCCCGGCCCCTGCCCGCCCCCTGCCCGGCCTCCCAGCGGCGGCGGCTGCAGCGCGGCGGTCCGAGCGGGTGCACCGCGGCG
GAGGAGGCAGCATCCCGCGGCGCTGACGGTCCTGGGGAGAGCATGGCGCCGAGGTGAGGCCGGGGCTGCGGGCAGGGGTT
GGGGGACGCCAAGATGCGGCTGCGGGGGTCGCGGGGGTCGCGGGGCTCGGCCGGGTCCTGCGGGGGTCGCGGGGGTCCTG
CGGGGTCTGACCCGTGCCTGTCCCGCGCAGGTGCCCCTGGCCATGGCCGCGGCGGCGGCGCCTCCTGGACGTGCTCGCGCC
CCTGGTCCTGCTGCTCGGGGTCCGCGCGGCCTCCGCGGAGCCAGGTAAGACCCGGGCGGGACGGGAAGGTTCGCGCCGGTG
CCCGCCGGCCTCGCCGCCCTGGCTGGGGTCCCCGCCCGGCTCCCTCACCCCCGCCCCGGCCGCTCTGGGTTCAGCCCCGGCC
CTGCCCACGCGCGCAGGAGGCGCCGGAGCCGGACGGAGCGGGGCGGGGTGGCCGGAGTCCCGCCCATTCATTCTCCCCGC
GGGGTCCGGGCCGGGAGGGGTGCGCGGTGCGAGCGGCCGGAGTCCCCGCGCGGGGACCGAACCTCCGCGGCCGGCGGGGT
CGTCGGTGCCGGGAGGGCTCGGGGCGGGGCCGCGGGGTCTCGGGAGACGCGGCCTCTGTCGCCGTCACGGCCTCGTCCCCG
CAGCGCCGGGTCCCGGGACTGACCGCGGGCGGAAGGCGGCGGAGCGCGGGGCGTCGTCTCCGCGTTTCCAACCTGTTGAG
GCTCCTCGCCGCGGGCCGGGCCTTGTGCGCCTCTGTCCCCGCCGCCCTCAGGCCTCTGTCCCCGCGCCCGCCGTGGTGTCGG
GTTGCGCGGTCCCCTTTGATGCCAACCGAGCGCCGCGCGGTCCGAGAACAATGCCCAGAAGCCGCTGCCCCGCCGGCCTCG
CCGCTTCCCGTGCGCGGCAAACGGTGCGCGGGGCCCGTGGGGACCTTCCTGCCTCCAACCCCGGGACCCAGTTCCCCACCA
CCTGCTAACCGGGGCCTCCTGCCTCCAGCCAGCCCAGCAGAGGCCCGGGGGAGGCGGCGGGGACGCGGACCCCAGGGGAG
CCCCGCCCAAAGGGCCTGGGTGCCTTGTCGCGGGTGGAATCCCACCCGGGGGTCCACGCAGCCAGCGCCTGCCTCCGACTC
G
19 chr10: 134864694-134865392
CGAGGACAGGGCCCGGCCCCACCCCGGGGCGAGTACGAGTTTGGAAGCAGTGAGGGCGCAGAGTGGGGCTCCATCTGGGG
AGAGGCCTCTCTCTGTCACCCGGCCCCGTGCGGCAGTTCAGGGTCCCAGCATCTCCCCTGCACTGGGAGGCGGCAGCCGTG
CCCGGGGGGCGCCCGTGGTGGGCGGGTGCAAGAGAGAAGGGCGTCGGGTGTGCAAGCACCACCGGCCTCCCATTAACAGC
CTGCCCGGGCTCCGCCCACAGGAGCCACTGTCCACGTAACCTGCGTCTCTTTCTTTCAAGGAAAAGAGAAGCCAGCCATGG
CCAGGACCAGCAGCAGGGCCCCCTGCTCACCCACCTCGGTGTCGGATGTGGACTCGGACGCACTGTCACGGGGAAACTTCG
AGGTGGGGTTTCGGCCTCAGAGGTCCGTAAAAGCCGAGAGAGCGCAGCAGCCTGAGGCTGGCGAGGACAGACGGCCAGCT
GGCGGGGCCTCAGACGTGGAGGCAGTGACCCGACTGGCCAGGTCCAAAGGGGTCGGCCCAGCCTTGTCCCCCGGCCCAGC
CGGATTCCAGAGCTGCAGCCCCGGCTGGTGCAGCGCCTTCTACGAGGCCGACTGCTTCGGGGCCGACGTCCACAACTACGT
GAAGGACCTGGGGCGGCAGCAGGCGGACGGGGCCCTGCCCGACGCCCAGAGCCCG
20 chr9: 129556229-129557670
CGAATGCAGCATCCCTGCGCTCGGCGCCCTGCTATTGGCCAGAGATGATCTCACCGCCTCCCGGCCTGCGCGCGCCTGATTG
GCCGGTGCGGGGATGCTGCGCTCCGCCGCCGGCGGGGCAGTCCTTCAGGCGGGCTCCGCGAGCGTGTGTGAGCCTGGGGGA
GCGAAGAGCCGACGCGCCTGGCATCTCCCAGGGGGCTCAGGGGGCAGGAGCGCGGAGACCCCCGGACAGGGTCTTAACCC
CTTCCCGCCAGCCCGAGCCCAGCATCCCCTCCAGCTCCCCTCGGCGTCCCAACCCCAAGCAAAGCCATCCCCAAAGGATGC
TCCGCAGAGAGGCGGGGTCCGGGTCCTCCCCTCCACCCCCTTCCGTTTAACCCTCTCACCGCCGCGGCGCCGTCCTGCACTG
CCCGGCCAGCTGGGCTGGGAGAGACCCGCGCCCCAGGCATGTCCAAGCCCACCTAGAGGGCGGGAGGGTGGCGGGCGCTC
TGGCCCCAACCCTGGCATCCGAAGCATCCCCCGCGTTTCCTGCTCCTCACTCAGTCTTCAGATCCCAGTCCGGCGCCCCCGG
TACAATGGGGAGCCAGGGTGCCCAGGTCAGCCGCAAGGGCCAGCGTACCAGGCGGAGGACCGGCAGGACGCCGGAGACC
CCATCTCCCAGTCCCCCCTGCCCCAGCTCTCTCCCTCCTGCGGAGGAGGCAGAAACGGACCGGCATCTACCGCAGCCCAGA
GTCCCAGGAGTGGCCGCCGAACCCTCACCCCGCGGAGCGCCTGGGCGCCCAGAGGTGAGGCTGGGGTGACCCCGCCCCCTC
CCCGGGTCGGCCGGGCCCAGCCCAGCCCGACCCTGCCGGGCGCCGCTGAGCTGCAGCTCCCCGGCTGGCTCTAGGGCCCCG
GGCGGAGCGGCCGGGGGTCCCAGCCCGGCGCGCGTGGCGGGGGCCGAGCCGCCCCCTCACCTGCACCTGCACGAAGGAGC
CGCGGTAGAAGCAGCAGGCGGCGGCCGACAGGGCACTCCACAGGCTGCACCGCTCGGTCATGGTGGGGCCAGGCTGCCGG
GGCCGGGACGGTGTGGGGGGGCGGTGGCCGGCGGGGCAGGGGCGCAGGGACCAACCGGCTAGGCACCGGCTGCGCGTGC
CTCTCAGCGGCGCGATTACCTCATCGCCTTGTCGGGGGAGGAGCGGGGAGGCGGGGGGGGGAGACCCCACCCTCCAGCCG
GCCAGGGGAGGGGAGGGGGCCATTGTTCCTCCCTCCGCCCGCCCCCCGCTCCCAGCCCCTCGCCCCCCGCCCTCCAGTTCCG
CGCGCTCGGGCTGCCTGGAGCTGCGGGCGCGGAGCGGAGGACCTGCCAAGGAGGGTATTACCGGGGGCAGCGGGATCGGG
TGGGGACCCATGGGCCAGCGAGGGGAGGTTCTGCCTCCCCCACTCGCCGCACCTCTAACAGACCGGGAGCG
21 chrX: 47305347-47306043
CGCCTGCGCGAGTCAGGACGAGCGCTTGCCCCAGATCTTGACGTTTCAGGCGGCCCCTCCTAATCCGGAACACGACTCACA
TTCCGTTGGTCTTGCCTTGACAGACGTGACCCTGACCCAATAAGGGTGGAAGGCTGAGTCCCGCAGAGCCAATAACGAGAG
TCCGAGAGGCGACGGAGGCGGACTCTGTGAGGAAACAAGAAGAGAGGCCCAAGATGGAGACGGCGGCGGCTGTAGCGGC
GTGACAGGTGAGGGCGGGCCCGGGAGGGCTCGGTTTCTGGAGCGGCTGCCGGGCACGGGCAGGGAGCCCGGACCGAAAGC
TCAGCTCCAGGATGGCTGCGCTTGGGCCCCGGCGTTCCCTGCCCGGAACCGGAGGAGTGGTTTGACCCGGGGCGAGACCAT
CGTCGACAGCGGGGGGGGGTGGGTAACAGGAATAGGCGGGCAAGGCTGCGGGTGATGGTTTCGCCCGCTGCTAGTGGTG
GTGCGCCTGCGCAGAGCCGGAAGCCCTTTGGTAGGCGGGACCCGACCGAGTGGTGCCGGGATTCCGTCTTAACCCCCGCTA
AGGTGTCCAATGACCTTTCCTTACCAACTGCGGGGAGTGTGTGGAAACGCGGTTCACTCCCCGGTTTCCTTATTCTGGATCA
GTGTCTGACCGCCCCCGCCGGGAGAATGTCAGGATTCTCGCCTCT

TABLE 10
Hypermethylated genes and CGIs in bladder cancer.

| | | Fold |
|---|---|---|
| Symbol | CGI | Change |
| MEIS1 | chr2: 66525936-66527140 | 4.76 |
| NR2F2 | chr15: 94688798-94689034 | 2.93 |
| SOX21 | chr13: 94152191-94153185 | 2.8 |
| MAB21L1 | chr13: 34950554-34951119 | 2.68 |
| OTX1 | chr2: 63134539-63134851 | 2.67 |
| MGC2752 | chr19: 63784504-63785085 | 2.64 |
| IRX3 | chr16: 52873104-52882105 | 2.63 |
| PAX9 | chr14: 36205265-36206099 | 2.62 |
| FOXC1 | chr6: 1549606-1560865 | 2.5 |
| DLX5 | chr7: 96488158-96489487, | 2.45 |
| | chr7: 96489900-96490182 | |
| NR4A2 | chr2: 156892636-156892878, | 2.45 |
| | chr2: 156893804-156894601 | |
| CNTNAP1 | chr17: 38089258-38089793 | 2.35 |
| FOXF2 | chr6: 1338049-1339169 | 2.35 |
| HHEX | chr10: 94441310-94441717 | 2.33 |
| PRKCZ | chr1: 2072175-2072389, | 2.31 |
| | chr1: 2106293-2106661 | |
| HOXA7 | chr7: 27164708-27165039 | 2.27 |
| MSX2 | chr5: 174091287-174092335 | 2.26 |
| SLC45A4 | chr8: 142288380-142288627 | 2.22 |
| FLJ44006- | chr2: 111591678-111597436 | 2.21 |
| BCL2L11 | | |
| MAP2K2 | chr19: 4059920-4060207 | 2.2 |
| SHH | chr7: 155288454-155292175 | 2.19 |
| chr13: | chr13: 49605587-49606020 | 2.19 |
| MAD1L1 | chr7: 2082894-2083307 | 2.18 |
| hsa-mir-196a-1 | chr17: 44074360-44075233 | 2.17 |
| NXPH1 | chr7: 8449658-8450236 | 2.17 |
| chr18: | chr18: 5186244-5187389 | 2.12 |
| C10ORF114 | chr10: 21828640-21829594 | 2.1 |
| SIX3 | chr2: 45014932-45016562, | 2.08 |
| | chr2: 45013398-45013616 | |
| TFAP2A | chr6: 10530308-10530634, | 2.08 |
| | chr6: 10498025-10498551 | |
| ISL2 | chr15: 74414579-74414893, | 2.05 |
| | chr15: 74419318-74422570 | |
| VAX2 | chr2: 70984709-70985764 | 2.04 |
| AMZ1 | chr7: 2724531-2724783 | 2.01 |
| CYB561 | chr17: 58865386-58865618 | 1.99 |
| HOXD3 | chr2: 176737660-176738187 | 1.97 |
| SIM1 | chr6: 101003802-101004342, | 1.95 |
| | chr6: 101002495-101002783 | |
| EFNB2 | chr13: 105943410-105943788 | 1.91 |
| GAD1 | chr2: 171384799-171385226 | 1.91 |
| MSX1 | chr4: 4915358-4915735, | 1.9 |
| | chr4: 4910534-4911092 | |
| FOXA1 | chr14: 37137198-37138958 | 1.88 |
| HDGFL1 | chr6: 22677564-22678684 | 1.85 |
| chr13: | chr13: 49595986-49600287 | 1.84 |
| chr16: | chr16: 87372445-87372706 | 1.84 |
| EVX1 | chr7: 27251165-27252762 | 1.83 |
| UNG2 | chr5: 54554812-54555385 | 1.83 |
| (CCNO) | | |
| ONECUT2 | chr18: 53254153-53259851 | 1.81 |
| HSF2BP | chr21: 43902100-43904249 | 1.8 |
| chr1: | chr1: 119350668-119352843 | 1.8 |
| FOXD3 | chr1: 63554983-63563059 | 1.79 |
| FOXA2 | chr20: 22514822-22515055, | 1.77 |
| | chr20: 22510737-22514104, | |
| | chr20: 22505518-22507240 | |
| GRIK2 | chr6: 101953488-101953856 | 1.77 |
| chr2: | chr2: 45249374-45251690 | 1.77 |
| SIX1 | chr14: 60178708-60179539 | 1.76 |
| HOXA9 | chr7: 27170441-27172987 | 1.75 |
| FOXD2 | chr1: 47671713-47671985, | 1.73 |
| | chr1: 47682300-47683607, | |
| | chr1: 47672249-47672972 | |
| NKX2-8 | chr14: 36122886-36123441 | 1.72 |
| ZNF274 | chr19: 63420090-63420541 | 1.72 |
| TAF4 | chr20: 60061693-60062033 | 1.71 |
| SIX2 | chr2: 45085286-45086054 | 1.7 |
| chr8: | chr8: 145909302-145910517 | 1.7 |
| PCDH7 | chr4: 30330303-30333940 | 1.69 |
| PITX2 | chr4: 111774415-111774953 | 1.68 |
| TCF7L1 | chr2: 85213468-85216104 | 1.68 |
| chr6: | chr6: 10492952-10493478 | 1.68 |
| SOX7 | chr8: 10624024-10624296 | 1.66 |
| HOXC4 | chr12: 52726910-52727810 | 1.65 |
| PITX1 | chr5: 134390992-134393045 | 1.65 |
| PCDHGA12 | chr5: 140790679-140792801 | 1.64 |
| chr6: | chr6: 170422815-170423038 | 1.64 |
| EPN1 | chr19: 60907042-60907388 | 1.63 |
| DLX6 | chr7: 96469320-96469736 | 1.62 |
| hsa-mir-10b | chr2: 176723195-176723460, | 1.62 |
| | chr2: 176720618-176720921 | |
| SIM2 | chr21: 36990064-36995761 | 1.61 |
| CACNA2D4 | chr12: 1844001-1845219 | 1.6 |
| HOXA5 | chr7: 27149139-27152087 | 1.6 |
| NCK2 | chr2: 105864220-105865009 | 1.6 |
| OSR1 | chr2: 19424445-19425131 | 1.58 |
| C17orf27 | chr17: 75936057-75936582 | 1.57 |
| (RNF213) | | |
| chr6: | chr6: 169051743-169052507 | 1.56 |
| EVX2 | chr2: 176652334-176656692 | 1.53 |
| ZNF503 | chr10: 76825135-76839606 | 1.52 |
| chr5: | chr5: 92931996-92934631 | 1.52 |
| KIAA1303 | chr17: 76404186-76404427, | 1.48 |
| | chr17: 76390187-76390410 | |

TABLE 11
Genomic distribution of methylated probes in bladder cancer.

| Probe location | % on array | % in bladder cancer |
|---|---|---|
| divergent promoter | 2.5 | 2.4 |
| downstream | 3.9 | 4.0 |
| inside | 52.4 | 52.3 |
| promoter | 26.0 | 25.9 |
| intergenic | 15.3 | 15.3 |

TABLE 12A
Overview of the CGIs differentientially methylated
between tumor and control urine.

| Symbol | CGI | Fold Change |
|---|---|---|
| HOXA9 | chr7: 27170441-27172987 | 16.53 |
| PENK | chr8: 57520681-57521969 | 14.69 |
| PCDH7 | chr4: 30330303-30333940 | 12.00 |
| KCNA1 | chr12: 4888847-4891432 | 11.89 |
| HLX1 | chr1: 219134071-219134808 | 10.46 |
| POU4F2 | chr4: 147778656-147781351, | 10.17 |
| | chr4: 147777682-147778033 | |
| LHX4 | chr1: 178464743-178471598 | 9.79 |
| FOXD2 | chr1: 47682300-47683607, | 9.67 |
| | chr1: 47672249-47672972 | |
| LBX1 | chr10: 102986025-102986636 | 9.44 |
| chr11: 115956893- | chr11: 115955870-115957122 | 9.03 |
| 115956940 | | |
| LMX1A | chr1: 163590111-163590435 | 8.86 |
| BHLHB5 | chr8: 65662303-65662699, | 8.61 |
| | chr8: 65661156-65661382 | |
| OTX1 | chr2: 63134539-63134851 | 8.22 |
| GADS | chr10: 26544390-26547440 | 8.02 |
| EVX2 | chr2: 176652334-176656692 | 8.02 |
| FLJ44006-BCL2L11 | chr2: 111591678-111597436 | 7.89 |
| ONECUT2 | chr18: 53254153-53259851 | 7.75 |
| ZIC1 | chr3: 148619591-148620015 | 7.33 |
| FOXA1 | chr14: 37137198-37138958 | 7.24 |
| DSC3 | chr18: 26875488-26877115 | 7.19 |
| SIM2 | chr21: 36990064-36995761 | 6.78 |
| MEIS1 | chr2: 66525936-66527140 | 6.74 |
| FEZF1 | chr7: 121727243-121727884 | 6.62 |
| ZNF503 | chr10: 76825135-76839606 | 6.57 |
| SLC35F3 | chr1: 232106922-232108080 | 6.34 |
| FEZF2 | chr3: 62331811-62332352 | 6.26 |
| NR4A2 | chr2: 156893804-156894601 | 6.20 |
| EN1 | chr2: 119329502-119332035 | 5.59 |
| PCDH10 | chr4: 134291113-134293078 | 5.53 |
| OSR1 | chr2: 19424445-19425131 | 5.23 |
| ZIC4 | chr3: 148591199-148594390 | 5.00 |
| CBLN1 | chr16: 47868915-47869809 | 4.92 |
| HMX2 | chr10: 124891898-124892607 | 4.89 |
| FOXB1 | chr15: 58083428-58085812, | 4.76 |
| | chr15: 58079172-58079459 | |
| chr19: 057648585- | chr19: 57648469-57649057 | 4.58 |
| 057648629 | | |
| DPP10 | chr2: 115635208-115637235 | 4.55 |
| chr7: 032768257- | chr7: 32768010-32768497 | 4.48 |
| 032768301 | | |
| C10orf114 | chr10: 21828640-21829594 | 4.44 |
| FOXD3 | chr1: 63554983-63563059 | 4.39 |
| chr5: 092933638- | chr5: 92931996-92934631 | 4.25 |
| 092933682 | | |
| TCF7L1 | chr2: 85213468-85216104 | 4.21 |
| VAX2 | chr2: 70984709-70985764 | 3.99 |
| CBLN2 | chr18: 68359955-68362770 | 3.97 |
| PCDHGA12 | chr5: 140790679-140792801 | 3.94 |
| chr21: 037857850- | chr21: 37857725-37858416 | 3.89 |
| 037857894 | | |
| HOXA7 | chr7: 27164708-27165039 | 3.79 |
| GRIK2 | chr6: 101953488-101953856 | 3.77 |
| NKX2-8 | chr14: 36122886-36123441 | 3.65 |
| SOX9 | chr17: 67627870-67631593 | 3.64 |
| chr18: 005186349- | chr18: 5186244-5187389 | 3.50 |
| 005186393 | | |
| SOX21 | chr13: 94152191-94153185 | 3.43 |
| SNRPF | chr12: 94776152-94776377 | 3.39 |
| MSC | chr8: 72916429-72917309 | 3.18 |
| PITX1 | chr5: 134390992-134393045 | 3.05 |
| SOX7 | chr8: 10624024-10624296 | 2.99 |
| chr2: 042182832- | chr2: 42182944-42183157 | 2.97 |
| 042182879 | | |
| TBX15 | chr1: 119333515-119333719 | 2.96 |
| SIM1 | chr6: 101003802-101004342 | 2.91 |
| PFKFB1-APEX2 | chrX: 55043085-55043710 | 2.89 |
| GATA2 | chr3: 129688183-129694961 | 2.86 |
| chr5: 122462228- | chr5: 122461778-122463450 | 2.82 |
| 122462286 | | |
| GPR103 | chr4: 122521018-122521740 | 2.81 |
| SIX2 | chr2: 45085286-45086054 | 2.80 |
| SFMBT2 | chr10: 7489383-7495345 | 2.79 |
| NXPH1 | chr7: 8449658-8450236 | 2.73 |
| CA10 | chr17: 47590175-47591465 | 2.71 |
| CNTNAP5 | chr2: 124498723-124499725 | 2.66 |
| ZNF274 | chr19: 63420090-63420541 | 2.62 |
| HOXD3 | chr2: 176737660-176738187 | 2.61 |
| PITX2 | chr4: 111774415-111774953 | 2.55 |
| OLIG2 | chr21: 33316999-33322115 | 2.54 |
| FOXA2 | chr20: 22505518-22507240, | 2.53 |
| | chr20: 22510737-22514104 | |
| DBC1 | chr9: 121170908-121172035 | 2.49 |
| PAX2 | chr10: 102497473-102499636 | 2.45 |
| MAB21L1 | chr13: 34950554-34951119 | 2.35 |
| CYP26B1 | chr2: 72224630-72228512 | 2.31 |
| CBLN4 | chr20: 54012011-54014085 | 2.26 |
| GAD1 | chr2: 171384799-171385226 | 2.25 |
| HHEX | chr10: 94441310-94441717 | 2.22 |
| chr9: 013268499- | chr9: 13268313-13269805 | 2.20 |
| 013268548 | | |
| FOXF2 | chr6: 1338049-1339169 | 2.17 |
| DLX5 | chr7: 96489900-96490182 | 2.08 |
| TBX3 | chr12: 113589232-113589931 | 2.04 |
| MEIS2 | chr15: 35174679-35174906 | 2.04 |
| PTGER3 | chr1: 71284813-71286392 | 2.04 |

TABLE 12B
Overview of CGIs associated with progression of NMI-BC.

| Symbol | CGI | FC |
|---|---|---|
| GPR103 | chr4: 122520930-122520974 | 1.81 |
| chr2: 042182832-042182879 | chr2: 042182832-042182879 | 1.96 |
| KCNA1 | chr12: 004890074-004890118 | 2.01 |
| HLX1 | chr1: 219134113-219134157 | 2.14 |
| DBC1 | chr9: 121171898-121171944 | 2.59 |
| GPR103 | chr4: 122521031-122521075 | 4.17 |
| TBX3 | chr12: 113609259-113609303 | 2.76 |
| GATA2 | chr3: 129693434-129693486 | 2.20 |
| TBX2 | chr17: 056833879-056833928 | 2.15 |
| TBX2 | chr17: 056835246-056835290 | 2.10 |
| ZNF577 | chr19: 057082979-057083023 | 2.08 |
| MSC | chr8: 072916695-072916739 | 1.82 |

### EXAMPLES

### Example 1

The CpG island hypermethylations as indicated in the Tables 1-5 are the result of comparative studies using well characterized patient samples. Both tumor samples and controls were analyzed. The DNA was extracted directly from the tumor and control tissue. DNA samples were analyzed using a CpG island microarray comprising 200.000 CpG islands (Agilent Technologies Inc.). The results obtained are indicated in the various Tables 1-5 as listed herein.

### Example 2

### Materials & Methods

### Patient Samples, Ethics Statement and DNA Isolation

Samples from 44 fresh frozen bladder cancer tissues were collected, representing 29 non-muscle invasive tumors (19 with FGFR3 mutation (NMI-mt) and 10 without (NMI-wt)) and 15 muscle-invasive tumors (MI). Tumor tissue of patients was obtained from the Department of Urology of the Erasmus MC, Rotterdam. The medical-ethical committee of the Erasmus MC Rotterdam approved the project. All patients gave written informed consent. For validation, 90 formalin-fixed, paraffin-embedded (FFPE) bladder cancer samples (27 Ta NMI-mt, 13 Ta NMI-wt, 10 Tl-mt, 14 T1-wt, 26 MI) were collected from the Department of Pathology. Usage of these samples was performed according to standards presented in “The Code for Proper Secondary Use of Human Tissues in The Netherlands” (http://www.federa.org). Tumor samples were included only if at least 80% of the sample consisted of cancer cells, as verified by H&E staining. DNA from tumor tissue was isolated with the DNeasy Tissue kit (Qiagen, Hilden, Germany), according to the instructions of the manufacturer. Commercially available normal human genomic blood DNA (Promega, Madison, WI, USA) was used as a reference in the genome wide analysis and the GGMA. DNA isolated from cells present in normal urine of four healthy persons of more than 50 years old was used as reference DNA in the GGMA validation assay.

### Differential Methylation Hybridization (DMH) and Data Analysis

DNA amplicons were prepared for hybridization according to the DMH (differential methylation hybridization) protocol described by Yan et al [Yan et al. 2002.) Methods Mol Biol 200:87-100]. Briefly, 0.5 μg of genomic DNA was digested with the four base (T\hat{ }TAA) restriction enzyme MseI, which restricted genomic DNA into fragments less than 250 bp in length, while leaving the GC-rich CGIs relatively intact. Subsequently, the sticky ends of the fragments were ligated to linker primers. Samples were then digested with the methylation-sensitive restriction enzymes HpaII and BstUI to increase the genome coverage and to ensure complete digestion. Reference samples were treated in the same way to generate amplicons. PCR reactions (20 cycles) were performed using the purified, digested, linker ligated DNA as template to generate final target amplicons, followed by coupling to fluorescent dyes (Cy3 in case of human genomic reference DNA and Cy5 in case of tumor samples) before hybridizing to the Agilent 244K CpG island microarray. The pooled amplicons were co-hybridized on the array. Using this approach, genomic DNA fragments containing unmethylated CpG sites in one sample (e.g. the human genomic reference DNA) were degraded by restriction digestion and not amplified. However, corresponding DNA fragments in the other sample (the bladder tumor) that contain methylated restriction sites were protected from digestion and subsequently amplified by PCR. Differentially methylated sequences were identified by comparing hybridization signals between fluorescently labeled tumor (Cy5) and reference (Cy3) amplicons.

All microarray data generated are compliant with current MIAME standards according to Brazma et al [Brazma 2009. Scientific World J 9:420; Brazma et al. 2001. Nat Genet 29:365]. Normalization, preprocessing of raw data and statistical analysis were done using Bioconductor packages in an R programming environment (http://www.r-project.org/). Raw hybridization signals were normalized by applying the within-array global “lowess” normalization method [Yang et al. 2002. Nucleic Acids Res 30: e15], which assumes that the bulk of the probes on the array are not differentially expressed. The normalization also includes a pre-processing step, as a result of which the normalized intensities were log 2-transformed. The log 2-transformed intensities were then subjected to further statistical testing to determine which loci were differentially methylated. The linear model “limma” was applied [Smyth 2004. Stat Appl Genet Mol Biol 3: Article3]. We performed the following comparisons: All tumors vs. Blood, NMI-mt vs. NMI-wt, NMI-mt vs. MI, and NMI-wt vs. MI, to find genes that have different methylation profiles across the groups compared. These comparisons are analogous to a classical two sample t-test analysis.

The p-value threshold was calculated using the Benjamini and Hochberg method [Benjamini et al. 2001. Behav Brain Res 125:279]. The loci corresponding to p-values less than 0.05 and a log fold change of either greater than 0.5 or less than-0.5 with more than 2 probes representing the same CGI were classified as differentially methylated. We left chromosome Y probes out of the subsequent analyses.

### Illumina Custom Golden Gate Methylation Assay and Statistical Analysis

We designed a custom Golden Gate Methylation assay interrogating 384 CpG containing probes representing 238 CGIs, 184 genes, and 46 intergenic regions. The selection of the probes was based on the differentially methylated loci in the genome-wide analysis. Probes were included to detect methylation in all bladder subgroups, but also probes that appeared to be specific for a certain subgroup compared to others, and some probes specific for progression or death of disease. The methylation assay is an adaptation from the Illumina high-throughput SNP genotyping assay described by Fan et al. [Fan et al. 2003. Cold Spring Harb Symp Quant Biol 68:69]. In short, non-methylated cytosines (C) were converted to uracil (U) by treatment with bisulfite. Sodium bisulfite modification of genomic DNA was carried out using the EZ DNA Methylation Gold Kit (Zymo Research Corp, Orange, CA, USA) according to the manufacturer's protocol, using 0.8 μg of FFPE DNA. The conversion efficiency was monitored by PCR with universal BS specific primers provided with the kit. Arrays were run at Service XS, Leiden, the Netherlands according to the manufacturer's protocol and as described by Bibikova et al. [Bibikova et al. 2006. Genome Res 16:383].

Data were analyzed with Illumina's BeadStudio Methylation module software. All array data points were represented by fluorescent signals from both methylated (M) and unmethylated (U) alleles. The average methylation value β was derived from approximately 30 replicate methylation measurements for each locus. The methylation level was given by β=(max(M, 0))/(|U|+|M|+100). The β-value (0≤β≤1) reflects the methylation level of each CpG site where 1 represents fully methylated and 0 represents unmethylated. At each locus for each sample, the detection p-value was defined as 1−p-value computed from the background model characterizing the chance that the signal was distinguishable from negative controls. Using this as a metric for quality control for sample performance, 6 FFPE samples (6%) were dropped from the analysis, because they either had very low overall signal intensities or >25% of loci failed. The significant differences were assessed by comparing the average β value per locus per group. Significant loci were selected on the basis of an average β value ratio larger than 1.4 and a difference between the average β value (AB) larger than 0.3.

### Validation of Methylation of Individual CGIs

We used the MS-SNAPSHOT method described in [Gonzalgo and Liang 2007. Nat Protoc 2:1931; Lurkin et al. 2010. PLOS One 5: e8802] to investigate methylation in urine DNA quantitatively. The method involves the bisulfite conversion of genomic DNA and the subsequent amplification of interested genomic region. This is followed by the methylation specific single nucleotide primer extension with CpG specific probes and finally the fragments are visualized on a ABI sequencer. To test the method, methylation of MEIS1 gene was investigated using normal, patient urine samples along with bladder cancer cell lines. In vitro methylated DNA was used as a positive control. A list of primers and probes used is given in Table 6.

### Results

### A Genome Wide Methylation Profile for Bladder Cancer

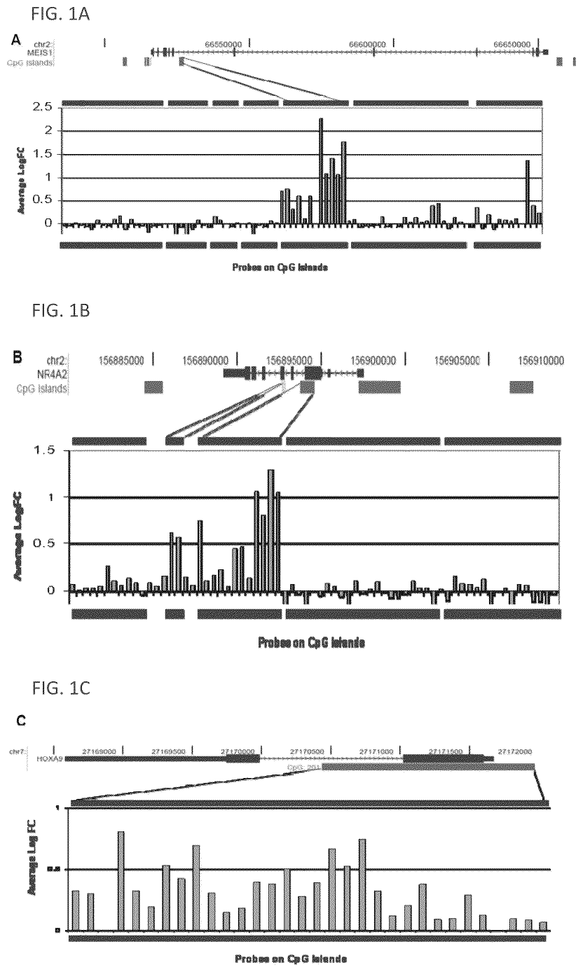

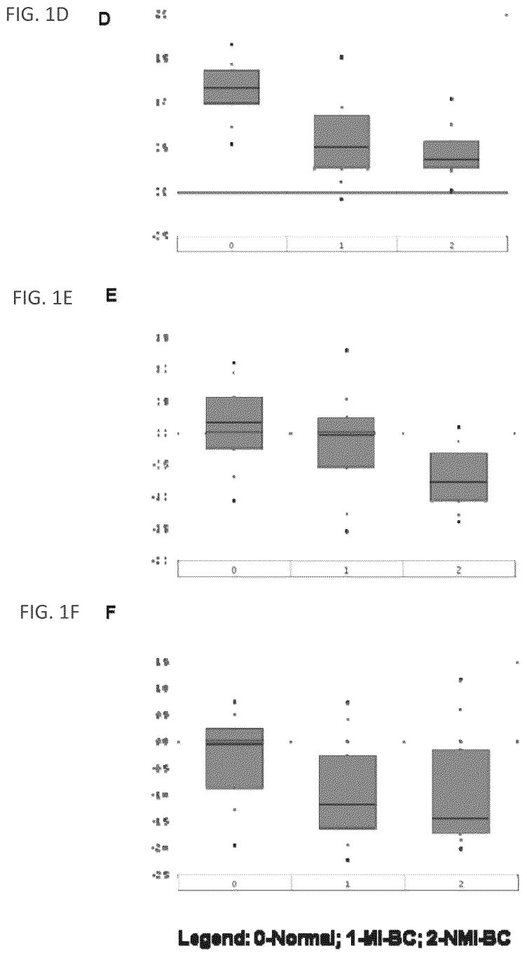

To investigate global DNA methylation in bladder cancers, we analyzed 44 bladder tumors on Agilent 244K human CpG island microarrays using differential methylation hybridization. We found that 729 different probes were significantly more methylated in bladder tumors compared to blood with a log fold change greater than 0.5 and a p value of <0.05. To select the most hypermethylated CGIs, we used as an additional criterion that 3 or more CpG dinucleotides should be highly methylated in a specific CGI. This selection generated a list of 82 CGIs representing 71 genes and 11 CGIs not directly associated with a gene according to the Agilent information file. These are shown in Table 10. The gene showing the highest degree of methylation was MEIS1 with a log fold change of 4.8 for the most highly methylated probe and an average log fold change of 2.2 across the 10 probes that were present on the array for this CGI (FIG. 1). We observed that for most of the significantly methylated CGIs, the adjacent CpG dinucleotides within a CGI were co-methylated. In contrast, CGIs neighboring a methylated island were usually not methylated.

Methylation of Intronic CGIs is Associated with Reduced Gene Expression

The 244 k array contains probes for CGIs as displayed in the UCSC genome browser (http://genome.ucsc.edu/cgi-bin/, build 19). Of the CGIs represented on the array, 26% is located in promoter regions. Many studies have shown that aberrant methylation of CGIs in promoter regions represents one of the most frequent epigenetic events associated with gene silencing in cancer. In our analysis we observed that there was no specific preference for methylation of promoter regions. Instead, methylation was distributed proportionally over CGIs in promoter regions, within genes and in intergenic regions (Table 11). Examples are shown for CGIs in two of the selected genes, MEIS1 and NR4A2 (FIG. 1). To investigate whether methylation outside promoter regions also repressed gene expression, we next compared genes with methylated CGIs with available genome-wide expression profiles of bladder cancer using the Oncomine database [Blaveri et al. 2005. Clin Cancer Res 11:4044; Dyrskjot et al. 2004. Cancer Res 64:4040; Dyrskjot et al. 2003. Nat Genet 33:90; Lindgren et al. 2006. Oncogene 25:2685; Modlich et al. 2004. Clin Cancer Res 10:3410; Sanchez-Carbayo et al. 2006. J Clin Oncol 24:778]. This information could be obtained for 54 genes. Out of these 54 genes, 22 showed promoter CGI methylation and a positive correlation with low expression was observed in 17 genes (77.3%). The remaining 32 genes showed methylation of CGIs within introns. Interestingly, 23 of these genes also showed low expression (71.9%) in bladder cancers. These data suggest that not only promoter CGI methylation but also methylation of intronic CGIs leads to reduced gene expression. The expression profile box plots of MEIS1, NR4A2 and HOXA9 are shown in FIG. 1.

Analysis of Individual Subgroups Shows NMI-Wt Tumors to have a Highly Methylated Profile

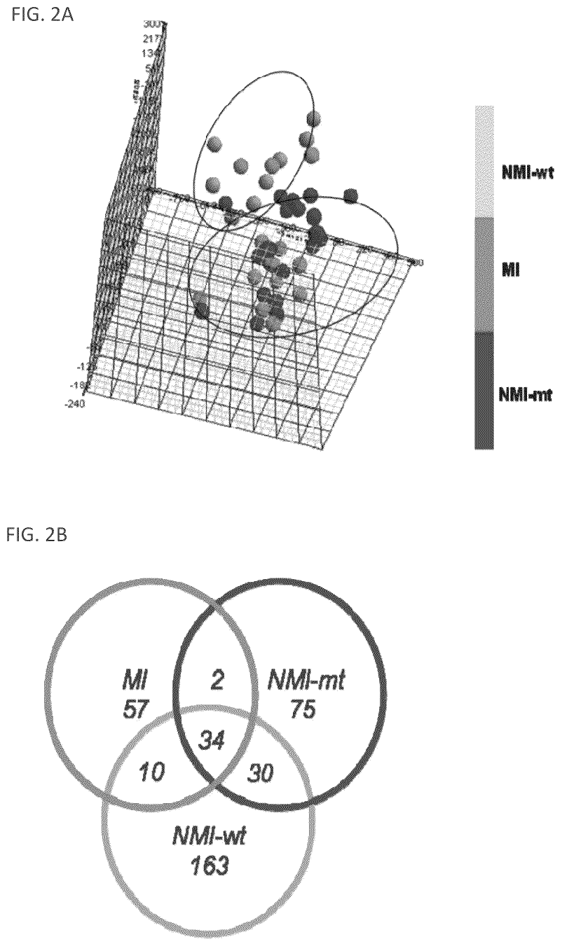

To explore whether global DNA methylation can identify subgroups of bladder cancer, we performed an unsupervised analysis by means of a principal component analysis (PCA) using all probes on the array (244K) (FIG. 2A). The first three components of the PCA explained 42.4% of the variability between the samples suggesting that there is a reasonable discrimination between the subgroups. This unsupervised analysis indicates the presence of two clusters of bladder cancer patients. The NMI-wt tumors (in green) clustered closely together and separate from the NMI-mt (in blue) and MI tumors (in red). Furthermore, we looked at the individual subgroups of bladder tumors. NMI-wt tumors showed a 2.7 to 4.6 times higher number of hypermethylated probes compared to the NMI-mt and MI groups (FIG. 2B). A list of genes differentially methylated between subgroups is given in Table 12.

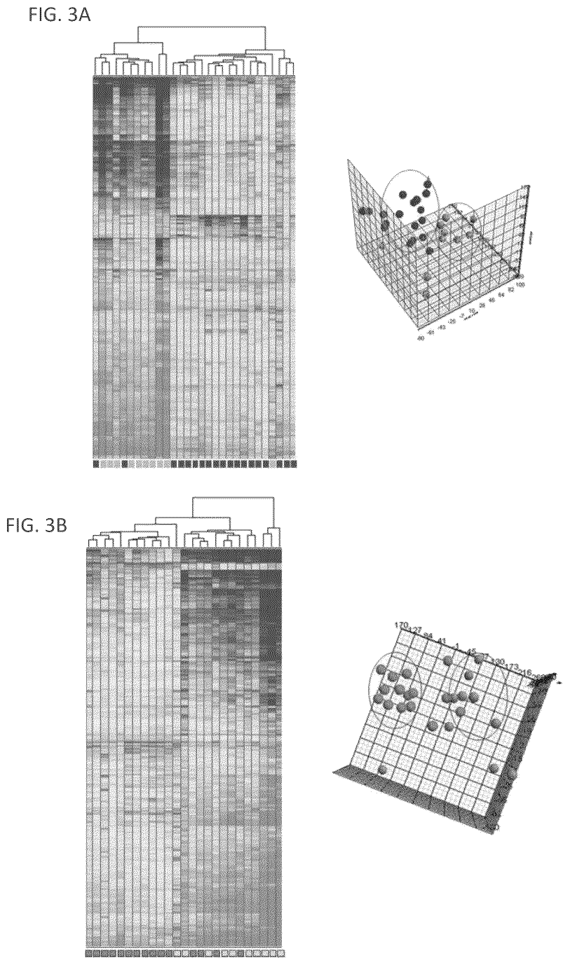

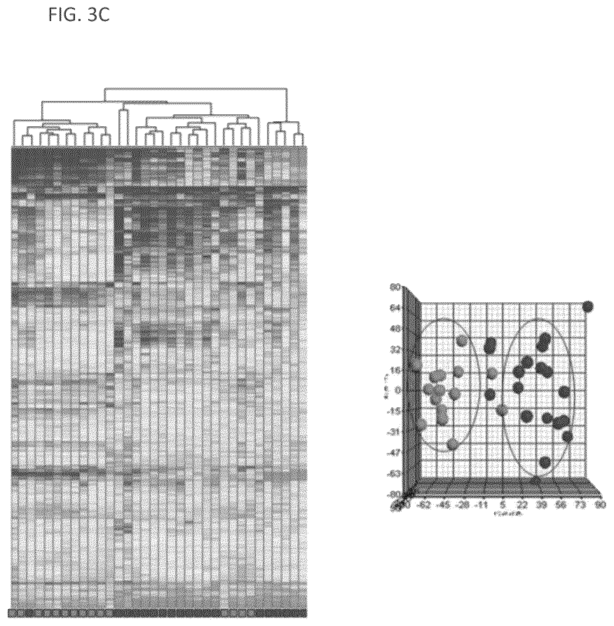

We next clustered the different subgroups of bladder tumors based on all probes that were differentially methylated between two groups with a p value of <0.05, regardless of the log fold change. For clustering of probes differing between NMI-mt and MI tumors, we had to use a p-value<0.5 because not many differences exist between these two groups. We employed a hierarchical clustering algorithm choosing Euclidian distance and complete linkage to organize the three different comparisons (FIG. 3). We then performed principal component analysis on the same data. The probes along with gene names and p-values are listed in Table 12. These semi-supervised analyses showed clearly that the different bladder cancer subgroups can be identified based on their methylation pattern.

### Genes Hypermethylated in Bladder Cancer Often are PcG Target Genes

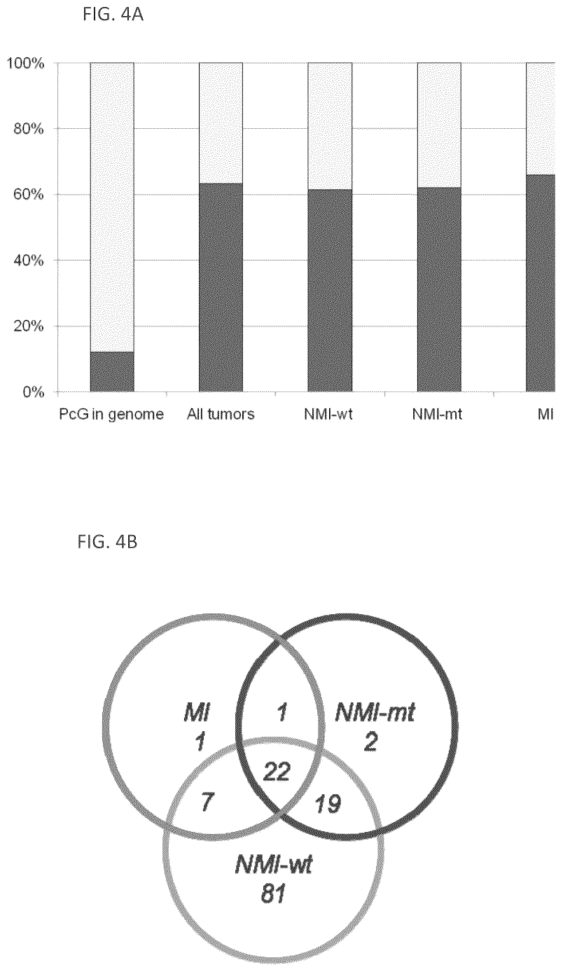

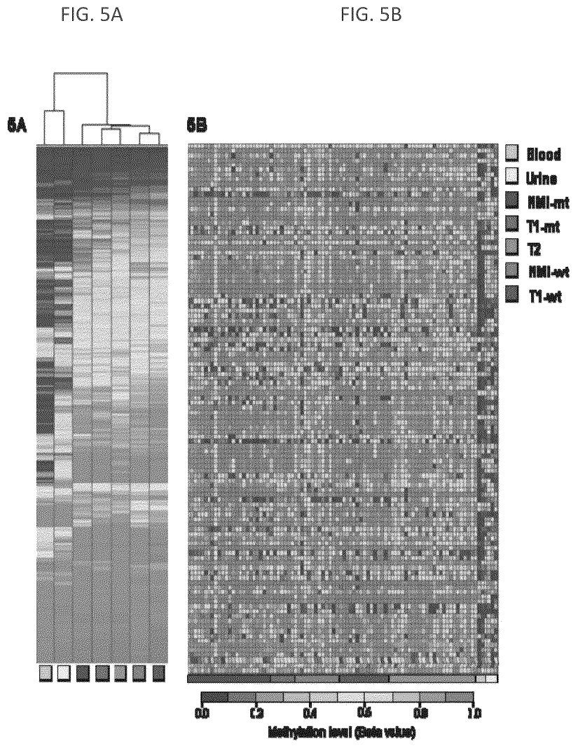

Polycomb group (PcG) proteins function as transcriptional repressors of genes that have been implicated in stem cell self renewal and differentiation. These PcG target genes have been shown to be particularly prone to DNA methylation in cancers [Ohm et al. 2007. Nat Genet 39:237; Widschwendter Met al. 2007. Nat Genet 39:157; Schlesinger et al. 2007. Nat Genet 39:232]. We investigated whether DNA hypermethylation in bladder cancer was more present in PcG target genes by matching our list of 71 genes from Table 10 with recent studies showing that 8-14% of all genes in the genome to be PcG regulated [Bracken et al. 2006. Genes Dev 20:1123; Lee et al. 2006. Cell 125:301]. Notably, we found that 63.3% of the hypermethylated genes in bladder cancer were PcG target genes, with a representation factor of more than 3 (http://elegans.uky.edu/MA/progs/representation.stats.html) (FIG. 4A). This was approximately the same for all different tumor subsets. Twenty-two PcG regulated genes were methylated in all three subtypes, while a striking 81 genes were specific to NMI-wt tumors (FIG. 4B). A list of the PcG target genes methylated in bladder cancer is given in Table 8.

### Genes Methylated in Blood Compared to Bladder Cancer

We also observed 21 CGIs that were methylated in blood but not in bladder tumors. These are listed in Table 9. We compared our results with a study based on methylated DNA immunoprecipitation (MeDIP), in which the genome-wide methylation pattern in blood was investigated [Rakyan et al. 2008. Genome Res 18:1518]. We found a 90% correlation with their study.

### Validation of Hypermethylation and Markers for Diagnosis and Progression

Next we set out to validate the selected significant CGIs on a custom Illumina platform based on bisulfite conversion of tumor DNA. The following CGIs were included in the custom array:
1) Differential methylation between tumors and blood (Tables 9 and 10);
2) Probes that could be of use as biomarkers to distinguish the subgroups. There were 7 CGIs representing 6 genes found to differentiate between NMI-mt and NMI-wt tumors. In the MI versus NMI-wt comparison, we found 17 CGIs representing 13 genes to be significant. No significant probes were found between the MI versus NMI-mt subgroups at a p-value<0.05. Therefore we decided to include probes from 6 CGIs representing 6 genes with a p-value<0.5;
3) CGIs that were associated with progression and survival. In all cases the CGI to be validated had at least 3 CpGs that were highly methylated.

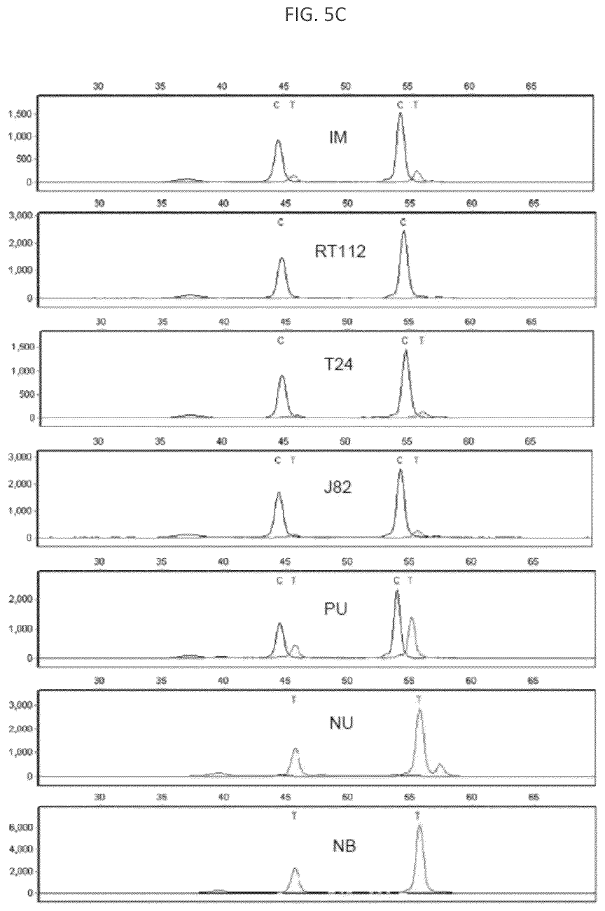

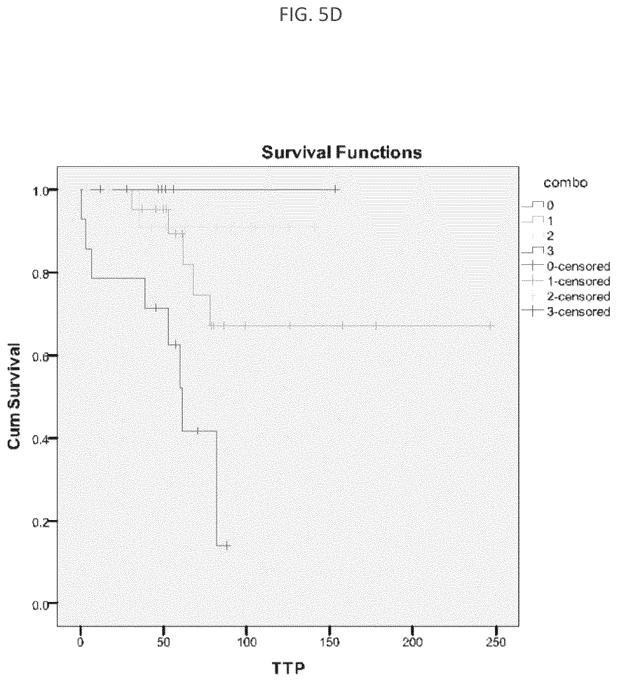

We employed a hierarchical clustering algorithm on the average beta values per subgroup with all 384 probes on the array choosing Euclidian distance and complete linkage. The analysis clearly separated the bladder tumors from normal urine and blood (FIG. 5A). We were able to validate more than 70% of the probes hypermethylated in all bladder tumors. For example, in the custom 384-plex GGMA, there were 205 probes which were methylated in bladder cancer but not in blood based on our Agilent array data. Of these, 147 probes were also methylated in bladder cancer but not in blood in the GGMA. These probes are shown in the heat map (FIG. 5B). We also included DNA isolated from cells present in urine of healthy individuals, which most likely are derived from normal urothelial cells. Eighty % of the probes validated against blood were also not methylated in urine. For the different subgroups of bladder tumors, the number of probes validated varied between 65-75% of probes compared to blood and 76-83% compared to urine.

CGIs that are differentially methylated between tumors and urine from non-bladder cancer controls represent potential biomarkers for urine tests (Table 12). We validated several of these using a bisulfite specific PCR followed by a Snapshot assay for the CpG. The results of these analyses are shown in FIG. 5C. We were able to detect methylation in urine DNA quantitatively using this method. We investigated MEIS1 gene methylation at two CpG sites using different probes (MEIS1 A and B). The black peak (Cytosine) represents methylation and red peak (Thymine) represents no methylation. In vitro-methylated DNA (IM), BC patient urine sample (PU) and bladder cancer cell lines (RT112, T24, J82) showed a strong methylation signal while normal urine (NU) and blood DNA (NB) were not methylated.

Next we analyzed the CGIs that were included on the GGMA as potential markers for progression. To this end the 6 best CGIs that were methylated in non-progressors vs progressors and similarly, 6 CGIs that were methylated in progressors vs. non-progressors were analyzed separately and in several combinations (Table 12B). The Kaplan-Meier curves depicted in FIG. 5D show that a combination of CGIs in the GPR103, DBC1 and GATA2 genes can significantly predict progression (p=0.001). Interestingly, methylation of the GPR103 and DBC1 CGIs was inversely correlated with progression and methylation of GATA2 was strongly indicative for progression. The DBC1 associated CGI overlaps with the promoter of this gene, whereas the GATA2 and GPR103 CGIs are located in introns.

### Discussion

We report an investigation of bladder cancer-associated aberrant DNA methylation. To our knowledge this is the first study that addresses genome-wide methylation in bladder cancer. The major goal of this work was the identification and validation of methylated CGIs that could serve as biomarkers for the early detection of primary and recurrent tumors as well as markers that are predictive of disease course. The genome-wide screen resulted in the selection of 177 CGIs that significantly differed in methylation in tumors when compared to blood. About 70% of these CGIs could be validated in a custom GGMA assay. This analysis also showed that most of these CGIs were not methylated in urine-derived DNA from age matched non-bladder cancer controls. This group of CGIs represents markers for urine based assays. Methylation of some of these markers was confirmed on urine samples. Another important finding is that a combination of CGIs from the GRP103, DBC1 and GATA2 genes is able to predict progression to muscle-invasive disease. Loss of heterozygosity or homozygous deletions of DBC1 (also called DBCCR1) gene (deleted in bladder cancer 1) have been observed in many bladder tumors, as well as methylation of the promoter. Methylation has so far not been correlated to disease course. In our study methylation of the promoter CGI of the DBC1 gene was associated with a decreased risk of progression. The role of the GATA 2 transcription factor in cancer is contradictory. Decreased expression was found to be correlated with proliferation in monocyte progenitors, whereas high expression seems to poise prostate cancers for a more aggressive phenotype and gain-of-function point mutations have been found in chronic myeloid leukemia. In bladder tumors a lower expression of this protein has been observed in MI vs. NMI-BC, which correlates with methylation and the positive association with progression. The GPR103 (QRFPR) gene encodes a G protein-coupled receptor and hence the protein is involved in signal transduction, however, but no further data about the gene or its product can be found in the literature.

Most genes discovered in the present study have never been shown to be methylated in bladder cancer or in other types of cancer. Some of the previously described methylated CGIs in bladder cancer that are associated with the SOX9, CDKN2A, TERT, DAPK1, EDNRB, and LAMB3 genes were also found to be methylated in our study, although only SOX9 and CDKN2A made it to the list of our 177 highly methylated CGIs.

To our surprise we observed that NMI-BCs with a wild-type FGFR3 gene have more methylated CGIs and the intensity of methylation is more profound than in the FGFR3 mutant NMI-BC and MI-BC groups. The difference in methylation presents additional evidence that the two subgroups of NMI-BC develop along different pathogenesis pathways. WT NMI-BCs also have more wide spread chromosomal aberrations than FGFR3 MT NMI-BCs. The extensive methylation in WT NMI-BC is reminiscent of the CpG island methylator phenotype (CIMP) that characterizes a subset of colorectal cancers (CRC). CIMP in CRC also identifies a subset of CRCs as it is associated with BRAF mutations, microsatellite instability and occurs more often in elderly females. No large-scale genome-wide study on DNA methylation has been performed for CRC, however, CIMP has been studied using a 1536-plex GGMA array.

When comparing the genes methylated in this study with those found by us, there was hardly any overlap. This confirms that CGI methylation is tissue and tumor type specific.

In line with previous reports in solid tumors, a high proportion of the genes we find de novo methylated in bladder cancer are repressed by polycomb complexes (PcG) in embryonic stem cells. PcG target genes are as much as 12 times as likely to be silenced by DNA methylation in cancer as non-PcG target genes. The association between DNA methylation and PcG has further been substantiated by the finding of interactions between DNMT1, DNMT3A, DNMT3B and several PcG complex subunits like EZH2 and BMI1. Another recent study showed that 49% of the genes methylated in colon cancer are polycomb targets in embryonic stem cells, agreeing with other reports showing that genes prone to tumor specific hypermethylation in colon cancer were more likely to be marked by H3K27 methylation in normal tissues than genes lacking H3K27 methylation. Another study reported that many genes hypermethylated in a prostate cancer cell line were bound by PcG in normal cells but lost PcG binding upon acquiring DNA methylation in cancer. In this perspective we found that 60% of the hypermethylated genes in bladder cancer are PcG targets in embryonic stem cells. These epigenetic alterations occurring in early development specifically in stem or progenitor cells might be bladder cancer specific and this strengthens the further understanding of bladder cancer tumorigenesis. Besides finding 92 commonly PcG regulated genes in bladder cancer subgroups, a group of 336 genes regulated by PcG complexes are found to be specific to NMI-wt tumors. Besides a possible CIMP phenotype being the cause for the extensive methylation in this group, another possible explanation for this finding could be that the cell from which the WT NMI-BCs originate is more closely related to the embryonic or bladder stem cell than the founder cells of the MT NMI and MI-BCs. In general it is thought that lineage-specific genes are activated during subsequent differentiation steps and during this process the PcG complexes will be replaced by cell fate specific transcription factors. Hence, fewer possible methylation targets will remain after every differentiation step.

Our data clearly shows that adjacent loci within a certain CGI are co-methylated, while neighbouring CGIs of a certain gene can have a very different methylation level. Significant association among the methylation states of adjacent CpG sites and a high frequency of short-range co-methylation was reported previously. Another interesting finding in this study is that our data shows no preference for methylation of CGIs in promoter regions compared to gene bodies, since the location of the significant CGIs is distributed over the genome in accordance to the distribution of CGIs on the array. The effects of promoter methylation on chromatin organization and gene expression have been well documented in various types of cancer. In our study, besides promoter methylation, we found more than 50% of all significant probes to be located in CGIs located within gene bodies. The influence of this on gene expression in not fully known. Previous reports in Arabidopsis have shown that gene body methylation was low in genes transcribed at low levels, higher in highly transcribed genes, and absent in genes that were not transcribed. By comparing our methylation results with gene expression studies, we show that for 70% of our genes there is an inverse correlation with gene expression, suggesting that intragenic methylation also contributes to gene silencing.

In summary we have identified hypermethylated CGIs in bladder cancer. The aberrant DNA methylation in bladder cancer seems to be very selective and could be the first step in tumor initiation, especially for the WT NMI-BCs. The study also illustrates an interesting biological finding: besides the promoter methylation intragenic methylation observed to the same extent in bladder cancer and it also appear to influence the gene expression. Many discovered methylated CGIs represent putative biomarkers for early detection in urine-based assays. The combination of CGIs from the GRP103, DBC1 and GATA2 genes predicts progression to muscle-invasive disease. Thus, the CGIs identified in this study will be of considerable interest for further investigation of their clinical and functional relevance and will provide advance insights into the pathogenesis of bladder cancer.

1.-27. (canceled)

28. A method comprising:
(a) obtaining a sample isolated from a subject at risk of or suffering from bladder cancer; and
(b) detecting an increased level of methylation within at least one CpG island of GATA2, TBX2, TBX3, MEIS1, DBC1, GPR103, ZIC4, HOXA9, PENK, and/or OTX1 in the sample.

29. The method of claim 28, wherein the sample comprises a tissue sample or a urine sample.

30. The method of claim 29, wherein the urine sample is a urine cell fraction.

31. The method of claim 28, wherein:
(i) the at least one CpG island of GATA2 is located between chr3:129688183 and chr3:129694961 and/or between chr3:129693434 and chr3:129693486;
(ii) the at least one CpG island of TBX2 is located between chr17:056833879 and chr17:056833928 and/or between chr17:056835246 and chr17:056835290;
(iii) the at least one CpG island of TBX3 is located between chr12:113589232 and chr12:113589931 and/or between chr12:113609259 and chr12:113609303;
(iv) the at least one CpG island of MEIS1 is located between chr2:66525936 and chr2:66527140;
(v) the at least one CpG island of DBC1 is located between chr9:121170908 and chr9:121172035 and/or between chr9:121171898 and chr9:121171944;
(vi) the at least one CpG island of GPR103 is located between chr4:122521018 and chr4:122521740, between chr4:122520930 and chr4:122520974, and/or between chr4:122521031 and chr4:122521075;
(vii) the at least one CpG island of ZIC4 is located between chr3:148591199 and chr3:148594390;
(viii) the at least one CpG island of HOXA9 is located between chr7:27170441 and chr7:27172987;
(ix) the at least one CpG island of PENK is located between chr8:57520681 and chr8:57521969; and/or
(x) the at least one CpG island of OTX1 is located between chr2:631384539 and chr2:63134851.

32. The method of claim 28, wherein detecting an increased level of methylation within the at least one CpG island of GATA2, TBX2, TBX3, MEIS1, DBC1, GPR103, ZIC4, HOXA9, PENK, and/or OTX1 comprises treating the nucleic acid sample with sodium bisulfite under conditions whereby unmethylated cytosines are converted to uracil, and methylated cytosines remain unchanged and performing methylation-specific PCR (MSP) on the treated nucleic acid using at least two distinct methylation-specific primer sets for the sequence of GATA2, TBX2, TBX3, MEIS1, DBC1, GPR103, ZIC4, HOXA9, PENK, and/or OTX1.

33. The method of claim 28, wherein detecting an increased level of methylation within the at least one CpG island of GATA2, TBX2, TBX3, MEIS1, DBC1, GPR103, ZIC4, HOXA9, PENK, and/or OTX1 comprises reacting the nucleic acid sample with reagents for detecting the level of methylation within at least one CpG island of GATA2, TBX2, TBX3, MEIS1, DBC1, GPR103, ZIC4, HOXA9, PENK, and/or OTX1 and performing a polymerase chain reaction (PCR).

34. The method of claim 28, wherein the detecting an increased level of methylation comprises detecting an increased level of methylation within at least one CpG island of GATA2, TBX2, TBX3, MEIS1 and/or DBC1.

35. The method of claim 28, wherein the subject is tested for the presence or absence of an FGFR3 gene mutation.

36. The method of claim 28, wherein the bladder cancer is non-muscle invasive (NMI) bladder cancer or muscle-invasive (MI) bladder cancer.

37. A method for diagnosing bladder cancer in a subject at risk of, or suffering from, bladder cancer comprising:
(a) measuring DNA methylation levels within at least one CpG island in GATA2, TBX2, TBX3, MEIS1, DBC1, GPR103, ZIC4, HOXA9, PENK, and/or OTX1 in a sample isolated from the subject;
(b) comparing the level of methylation within the at least one CpG island from the sample of the subject to the level of methylation within the at least one CpG sequence of GATA2, TBX2, TBX3, MEIS1, DBC1, GPR103, ZIC4, HOXA9, PENK, and/or OTX1 from a sample from a healthy control subject; and
(c) detecting an increase in the level of methylation within the at least one CpG island in the sample of the subject relative to the sample from the healthy control subject, thereby diagnosing the subject as having bladder cancer.

38. The method of claim 37, wherein the step of measuring DNA methylation levels is preceded by the step of extracting DNA from the sample.

39. The method of claim 37, wherein the sample obtained from the subject at risk of, or suffering from, bladder cancer comprises a tissue sample or a urine sample.

40. The method of claim 37, wherein the urine sample is a urine cell fraction.

41. The method of claim 37, wherein the urine cell fraction is obtained by filtration or centrifugation of the urine sample.

42. The method of claim 37, wherein the measuring DNA methylation levels comprises measuring DNA methylation levels within at least one CpG island in GATA2, TBX2, TBX3, MEIS1 and/or DBC1.

43. The method of claim 37, wherein the subject is tested for the presence or absence of an FGFR3 gene mutation.

44. The method of claim 37, further comprising performing a cystoscopy and/or transurethral resection on the subject.

45. The method of claim 37, wherein detecting an increased level of methylation comprises treating the nucleic acid sample with sodium bisulfite under conditions whereby unmethylated cytosines are converted to uracil, and methylated cytosines remain unchanged and performing methylation-specific PCR (MSP) on the treated nucleic acid using at least two distinct methylation-specific primer sets for the sequence of GATA2, TBX2, TBX3, MEIS1, DBC1, GPR103, ZIC4, HOXA9, PENK, and/or OTX1.

46. The method of claim 37, wherein detecting an increased level of methylation within the at least one CpG island of GATA2, TBX2, TBX3, MEIS1, DBC1, GPR103, ZIC4, HOXA9, PENK, and/or OTX1 comprises reacting the nucleic acid sample with reagents for detecting the level of methylation within at least one CpG island of GATA2, TBX2, TBX3, MEIS1, DBC1, GPR103, ZIC4, HOXA9, PENK, and/or OTX1 and performing a polymerase chain reaction (PCR).

47. The method of claim 37, wherein the bladder cancer is NMI or MI bladder cancer.

48. A method for predicting the recurrence or progression of a bladder cancer in a subject at risk of, or suffering from bladder cancer comprising:
(a) measuring DNA methylation levels within at least one CpG island in GATA2, TBX2, TBX3, MEIS1, DBC1, GPR103, ZIC4, HOXA9, PENK, and/or OTX1 in a sample from the subject;
(b) comparing the level of methylation within the at least one CpG island from the sample of the subject to the level of methylation within the at least one CpG sequence of GATA2, TBX2, TBX3, MEIS1, DBC1, GPR103, ZIC4, HOXA9, PENK, and/or OTX1 from a sample from a healthy control subject; and
(c) detecting an increase in the level of methylation within the at least one CpG island in the sample of the subject relative to the sample from the healthy control subject, whereby an increase in the level of methylation within the at least one CpG island in the sample of the subject relative to the sample from the healthy control subject indicates that the bladder cancer in the subject is likely to progress or recur.

49. The method of claim 48, wherein the sample comprises a tissue sample or a urine sample.

50. The method of claim 48, wherein the urine sample is a urine cell fraction.

51. The method of claim 48, wherein the urine cell fraction is obtained by filtration or centrifugation.

52. The method of claim 48, wherein the subject is tested for the presence or absence of an FGFR3 gene mutation.

53. The method of claim 48, further comprising performing a cystoscopy and/or transurethral resection on the subject.

54. The method of claim 48, wherein the bladder cancer is NMI or MI bladder cancer.


SEQUENCE LISTING (329 sequences)

SEQ ID NO: 1 - DNA, 550 bp, Homo sapiens
  source 1..550: mol_type = genomic DNA
cggatgtgcgtggactctgcgggcggggcgcccgtagtctccctccttcgcaccccgggcgcctgggaacctcctcgcgcgtttggccactcgctgctcctggcagtaggggcttcgggccccgccgtgtccccacccaggtatggggatgcggagttgatgggtgcggggcgctgggagggggcagatgaaaggggcagtcgaaagggggccatgcgcgccgagcgcagccgggtcattagccgcgggtgtgagcggcggggcaggcttatcgcccatccaggcctggcgggcgggcggtccccgacgcggctgccgccgccgcccgcggcagagggcacggcggttcccacgctcgggccctggttcgggggcgttctcgggacggcgtggccgcgctcaccaccaccgcggaggccgggctgtaattaggataattgcgctgctcccagcctggggcagctcaggacccggctgcaccgcgctggcccgggacgggcgggggcagggggtggcggggcccgagcgatacagcccctggcgcagtccg

SEQ ID NO: 2 - DNA, 1209 bp, Homo sapiens
  source 1..1209: mol_type = genomic DNA
cgcccagaccggccgtggcggggccccggcgccacgtgctgctcatggccaccacgcgcaccggctcctcgttcgtgggcgagttcttcaaccagcagggcaacatcttctacctcttcgagccgcttggcacatcgagcgcacagtgtccttcgagccggggggcgccaacgccgcgggctcggccctggtgtaccgcgacgtgctcaagcagctcttcctgtgcgacctgtacgtgctggagcacttcatcacgccgctgcccgaggaccacctgactcagttcatgttccgccgggctccagccgctccctgtgcgaggaccccgtctgtacgcccttcgtcaagaaggtcttcgagaagtaccactgcaagaaccgccgctgcggccccctcaacgtgacgctggccgcagaggcctgccgccgcaaggagcacatggccctcaaggcggtgcgcatccggcagctgagttcctgcagccgctggccgaggacccccgcctggacctgcgcgtcatccagctggtgcgcgacccccgggccgtgctggcctcgcgcatggtggccttcgccggcaagtataagacctggaagaagtggctggacgacgagggccaggacggcctgagggaagaggaggtcagcggctgcggggcaactgcgagagcatccgcctgtccgcggagctggggctgcggcagcccgcctggctgcggggccgctacatgctggtgcgctacgaggacgtggcacgcgggccgctgcagaaggcccgcgagatgtaccgcttcgccggcatccccctgaccccgcggtggaagactggatccaaaagaacacgcaggcggcccacgacggcagcggcatctactccacgcagaagaactcctcggagcagttcgagaagtggcgcttcagcatgcccttcaagctggcccaggtggtgcaggccgcctgcggccctgccatgcgcctcttcggctacaactggcgcgggacgccgccgccctcaccaaccgctcagtcagcctgctggaggagaggggcaccttctgggtcacgtaggggggccggggccccgtatgcccctcctcgtgaaaggcctgccccgtctttctgccgcagccctcgcagagggcgggtgcacagcgccatgacgggcagcgcctcctgtagcagtagggcccccagccagcgctccagccaa

SEQ ID NO: 3 - DNA, 316 bp, Homo sapiens
  source 1..316: mol_type = genomic DNA
cggggtcctcacggctcccgcaggcagccggcccctgctgctgtcttcaaacagcctcccgggggaccgctctctccgcgtgctgagcatccggccgggggacttctctcgctccagggggcggccagagacttgtccctgcgcagctcggtccgcccctcgcggtagcggtggggtgtgcttggctctcgcgggtggctggggccttcgggcggcgctgggctggaggccacgcgcttggtgatgtgctcgttgtacgtgggtgggcctcgcttgttggggctgctggcgacacaagagaacgtagggagctgcg

SEQ ID NO: 4 - DNA, 1209 bp, Homo sapiens
  source 1..1209: mol_type = genomic DNA
cgagaggacccggaccccggcggggaagggcgccctcccccggcctcgcggccgccccaaagcacatccccttctgccggccccaagtccccgcgcgtccacggcgcccctgtgcccacgggagagcacccccgcccagatggaaaagggggtagaccgggcagagcgagaggcggctttggctcgtggggagcggcgacccaacccagcgcgcatcccgcaagccctcccggctgccccggaccttccccctcgctacctcggaacagggtctgcccgggggctgctgcacaaagaggggtggtcgagggagacgccaaatcgttaagacggggagcccgtttcggccatcttggaagagaggaaagggaaaaagggacagaaaaaagggagaaagaaaggcggacggggcaagctgggctagggactgacggggcgcggggggtgcgaaggcaggatcggctcagagccccccctcccatcggggcttgcgaagaacggccggcccccatcccaatccccagagggttacaggccaggagagggaaaggcagggggagtccgctccgcttacctgggttcggggtccggtgggtctcggggagggggggatgggagggagggagggaagggaggggagggggccgcagccgtgtcgctcgcccgggcggcgggaggggagaaacctacggtaagaaaggagtttgtgaaagcggcttggggtgggagggagaggggaggggaggggaccgaggggggaggggagggaccggagtgtgggggggagacaaaatggcttttttcctccagacaaggtaggtcccgcccaccactcacccacgtgacctcattccttcaggatgctggcagagacgggaagaggagggggaggctggcgcggccgctccccgccaccggctgccgggtccctagccaggacgagacccctcccttcccatgcggccgccccgaccccggaggatggatctcttatgttaccttccgcagttagcccccctcccttcaaaatatgggttccccccactgacactgtgatgcccatccctaactggggcgcctggaaaatggtgaaatggggactgggctgaaaggtggctcgtggtcctccacgccccggcctagaccaaagggcgcggggtccggcgggggacgcgacgccttcagccaggcgtccgagcgattttcatt

SEQ ID NO: 5 - DNA, 1209 bp, Homo sapiens
  source 1..1209: mol_type = genomic DNA
cggaggcggcacagctggagcccggattgtggcacgccgtcaccgtgctgctccggggaatcccgacccgctccctgcgaaagcgtttccgaacgcgaacccagagcctgtgaacgcgccggcaagcccccactcccccaccgccgcccgtcgcaggtgggcccgtcctaggggtccttcctgcgctctaccccgtctctcaagtcactcagtcgatcgccccgttcacgctcccgtgatcccagacatccataaccacgatctcgcctccatgcacatccaacgcacgacggtgcacaacgtgcacctgacttctgcggaccaggtgtctcaagcgtacagcggccacccgcggaaccgcggcccggggccagtgagtcgtctgcagctccccgggttaggggattccccagaactccgggaaagtcacccgaagtccatccgggatgcggccttggttctcggccgcgtatctcgcccttaggtgcagaacgacgcccttccagggcccacagctgccaggctgggccttgccctcgcatcccccgggaagaccagggacggggccacacaggccgtggctgcggagacgcttccccgggccaccccgcgaccaggagggagtcgagccgcccgctctcccgcgtcccccgccccatcccagccagttggccccaccctgcgcgggataattgggacgggagggaggcgacgggagggcggcggctccagagagactgcgcgcctgtcagcggcaatttgtttaagtggacgggacgggccgggccgctgcgggctggggtcaccgaggccgcgcccccaccccaaccagacctgggaccgcgggggagcccggtccggccgctaaaccgggctggctggcgccagggctccgggaggtgcggtccggcggggaagccgtgatgggaagcgactctgtccagggagtgtccttcaccaccacactcctcacgtccaggcagtgatgacggcctggcggcaccctcacagcgggcccatagcacggggccacacacgtcccctgagcttagcctgggcacattcgtctgccaccgagggcttaagccagtctgcagcccgcgccccgtcactcggacgcaagtccgtcgtccgctctgccacgcggccgcacagcccggcttcctgctgcccactgcccgcggggtcacgcaaccccggccctgcaca

SEQ ID NO: 6 - DNA, 688 bp, Homo sapiens
  source 1..688: mol_type = genomic DNA
cgatttccgcagatttcctgctggaacgttctctcgggcctcaagttttcctgagaggacgacgtggagagtggagacaaagatgctcaaaagccaggaaatcgcaggctcggaagcccccacgcatcctctgaacgcagcgccatctcggggctgcggcgggaccaagcgggacgcttgcaggctgtcggcggcgggggccgcgagccgccggcgtaggccgctccagctggaagaccccaccgctagggttccagggggctgcggacgcttccgaggggcccaaggcgaaagaacgcgcccgcttgtcggccacgtgaccgtccccgtccgcgtccgcagcgaccacgtgactgtgcgtccgcggcgtcgattgactgggagctccctggccacgccctaagcgccagccccgggcttcggggggcggtgctgctgcggttgccatggcgtcttccgagcgcgcaggcggggcccagagtagcttttcgggtatttgctaccggctctcggtagggctggtcacctttgcttgctgagcttcgcgccctgcgtcctttggtccccagcaacttgcagggcagccgtccctcatgaggccctccagcgaatgccctaaagctccgaagttgctccggaggaaacaggcttgctccgagggtgcacgtccttactgcccgtgacgtcg

SEQ ID NO: 7 - DNA, 511 bp, Homo sapiens
  source 1..511: mol_type = genomic DNA
ctgctgcacttgtaaagtgggtgcttctcagcttcatggtttgaggggtgcagccagcttcggcctaagtggtgttgagggtcgaggacccagcacccccacacgggaagcgcggtgtcccgccttcaggctggagtactctggcaccctctggggtcccgaagtgcctgagtacctgtttgagccgcgtgtaggtgactgcgcacctgtttgaactgcgtgtaggtgaccgcgcacctgtttgagctgcctgtagctgaccgggcacctgtttgagctgcctgtaggtgaccgcgcactgtttgagctgcctgtaggtgactgggcacccgtttgagctgcctgtaggtgaccgcgcacccgtttgagctgcctgtaggtgaccgggcacccgtttgagctgcctgtaggtgaccgggcacccgtttgagctgcctgtaggtgactgggcacccgtttgagctgcctgtagtgaccgcgcacccgcttgagctgcctgtagctgaccgcg

SEQ ID NO: 8 - DNA, 1078 bp, Homo sapiens
  source 1..1078: mol_type = genomic DNA
cgcgttgtaggggtagccgtagtaggccagtgtgtaggggtcgcaggagtacacgtagctgggctgctgcgctgcctcagccgcgccgccgcccctggctgccttctggtagcgcgagtactgctcctgtccacgggcttggccagcgtgacctccaggcacgagccctccagctcagtgccgttgaggttgttcatggcatgcacggcatcctcgcggctggtgaagtgcacgaaggcgtagtcgcggatcttcttgacgcgctccacgcagccggggttgaactggccgaagctcttttgatggtgtcctcggtggtctcgatcatgaggttgcgcacgtagaggatcttcacggtctccatcacgtcctcgtccacgtcgatctcaggttcggcccagtccacggcgatctggtggccccacagctggatgcggccaggcatgagcttgcggcgagccatggcagccggcggtggctctcgtactccacgaaggcgaagccgcggttcttcatcttgtcggccgcgctggcgtagacgatcacgtccagcacgccctcggtgaccttggcaatctcctccaggatttcctcgcgcttcttcatcttggggatcccgccgatgaagaggcggcagttgtcccgctgcagcacacgccgagcaggcggcccgggcggatctcgtagttgttgagctcacgcactgcgcgcttggcctcgtgcttgtggcagtacatgacgaaggcgtagccgcggttcttgccgtcaaagtccatcatgaggcgcagctcgtagatgcggcccacggcctcgaaacgggcaccagctcgtcctcgtacacgtcgcgcgggatcttgcccacgaagacctcgcagccacgctgcgggtgcgggccctcccagccgggcggtgggccgccgtacttgcgctgcccgttctcttgcaccatgctgtagcccgtgcgctccatcagcgccagcagtgctgctcgttgggcgcgcccgccacgccctcgggcaccttggcggaggacccggcggccgagtcactgctcatggctgcggtggaatcctctgcg

SEQ ID NO: 9 - DNA, 1167 bp, Homo sapiens
  source 1..1167: mol_type = genomic DNA
aagtctggagataattatgtcgtacagtcgcaaacattattccgttcttactgtaaacggccccggccacctttacgagaaaccaggaaacttctgagagttactagcagcgtttacgcgggcaaacgagttctttttctttctctcccggattgttcgaagtatctatcgggcggcttcgatgccaggttcagaggcgcgccagggagagggcgccccgcagaggagcgcagcggagaggcctacgcaggtccccggtgcccgcggccctcggaggccgggccctgcgtcttggccagcactgggtggcagctgaggctggtggcccggagccctcgcggccgcgggcaggccccttcttgggcagggtcgggcactcccgctgtccagggctcttcggcaccctccttccaatcaggtcgctctcccctgctccccagactcaactcctccgaagctgctccaggttgaatgtgaccgctaggccgactccctgggcccgcgagcagttctcgaaaggtgcggactgagccctttctggggtggggtgcgggttggttctcgcaagtgtgacccagggtgaacttgctatttcgggtcccgggtgctgcagggccaggagaacagctgggatgggggaccccgcctccaccctcgggccggcacgtccgcgccctgtcaggtccccctccctcctctatgatggccaaggcgtgcgccagggctatccgggaaccttgtaaggcctcgtgctggcacctaaccccactcgcggcacacttcctctatgtagtctgcggccccgcctgccaaatagagtgaccagtgcagggacagaatgccaggctggtggccgaccgcctgagggacaaaggcgagcattcacaagccaacagcagacccctgccccccatatttccatttcgctcaggcttttaggacaaaatcaacaaggccgcagagtggtgcaggcgctcaccccgggtgcagcctggggagccactggttccgcgaccctgggcatgaaactcctcaagggcggccctcgagacgcaggggagaggatgctgccggcgcctgcccgagggcttctctgcgggaagcgggcaggcaccccaccggagtcattgccgggaccctcagcgcaacgcgggcctgttcctctcg

SEQ ID NO: 10 - DNA, 751 bp, Homo sapiens
  source 1..751: mol_type = genomic DNA
cggagatggcgcgtctggattcagggagatcgtgcggccagcttccctccctctgctctggatgggggccggccgccgccgctcactcacggctctctctctcccgctgtgcctgcaggtgtggccgcgacgatgccctcgctgaggagcgcatgcggagaggccagaacgccctgccagcccagcctgccggcctccgcccgccgaagccgccccggcctgcctcgctgctgagacacgcgcctcactgtctctctgaggaggagggcgagcaggaccgacctcgggcacccgggcccgggaccccctcgcgtccgcagcaggtgtgcaaagggaggcagccgtggagcaggcgcagagtgagacttctctgggtgccagaggacagcgaggacacaaaccgcgcctgggctgctgtgttctgacctgagcgggcgtgggcggtcgggcggggcgggaggaggtagcctcctctgctcctctcccgctcgcggcgccccacgccacggtggctccgggccccccaccgttaaaacggggtccgcgtgtcctgtacgcagcgtgttgctccgcaaaggatgacagagctgccacggcagtgcgaggtgctgcttttaccgtagacgtgccttttcgtagctttagcgtagagcttgaggcctgtggcttaggccgggtggtctcgtgctggtggggtctgcgtttgcccctgcccctccgtcagcttcaccttctcgtgccttcttctgcg

SEQ ID NO: 11 - DNA, 734 bp, Homo sapiens
  source 1..734: mol_type = genomic DNA
cgtaacgggcgtgggtcgtccgtcgcgccgcccggcgaggagtgggctggcggcggtagctgtcgcccgcttggttgcgtgaccgcggggtccgcgtccgctccctccacccttcgcccttcgccctcgcctcgttccggcctccgcggcccagcaacggccgtcatggtgccgtcggcgctccctgcgcggccccgctgagcctcggtgcggcggcgagcgcggtcgagatcgccatgcctacccgtgagtggccggccgagggccgggggcgttgggaggcgactgcctgcgcgacggcttgggccgggtactcctcccgggcggctggggaggctgggccgcgctgagcagagccaggaagtgtctgcgagcggcggagttagtttcgtttcggcttagccttccggagatggcgtgagcccgttgcacggatcgggaaactgaggcccgaacgttaagggaactgcgggacgcgctgttaggaggcggcagagctggtggtaacctgacccgtgggatttcaaagttaaagcccttttcccacggccacacggcttcgtgggactttatggaaatattgaaaataactgcggtaccattgtcagtcccttctcacctcgggccggacgtcgctagtggagcgccggccccgagtcccttcccgcggccgccagacagacccccgacagcctaactcggcgtccgtatcgccgtcggggcggctgaagtcg

SEQ ID NO: 12 - DNA, 279 bp, Homo sapiens
  source 1..279: mol_type = genomic DNA
cggaggcacgaaccaagcagcctagaagacaaatgcgctgctcggagagactgccgcggcaaccaactggacaccccaagagctcactcctccgcggttttatattccgacttgcgcacaggagcgggtgcgggggcgcagggagtgtgggtaacaggcatagattccgcttgcgcaatacgtggtaagaaaccagctgtgaggggctggcccaacgcagagcggcgcgaggtccgcttggcatctacaacaatcgaggggctagtccagaagttcaag

SEQ ID NO: 13 - DNA, 1209 bp, Homo sapiens
  source 1..1209: mol_type = genomic DNA
gctcagctagtaagtgaggagctcagctctgaagctgtaaccaagaccgcgcgggcaggaggaaaggacacttcagcgctctgcggttgcaaacacacccctgcgggtgctgccacttagcaaagtctaggttcccgcttactcgcacacgcatcctgacttcccccaaccctggaactacctaccaggagcggagaccagcagaccgacagcacgcacatgatccccataagctgaatggccgtctcggtcgtgatgcggccccactgggcactggactgagatgccgtggccttggccggcagcgggacaccagggccttaatggtggccaggttgcaggaaaaggtgactgtcagcgccaagagccccaggaaggcaaaggcagaggcgaagaaaaggttgccccagttatgcgaagagctagtcccgttgccccctcgcccggtgctgatgaagcaccacgtcccggccactggacggtgtactggcccacgcccagcaccggcagcagggcgaaggcgagcacggccagccacacgccgagcagcacagcgcgggtggcacgcgtcttcatgtggctcgcataccagtgcggcgccctgatggccagcgcccgctcgacggccatggcgctggcgatgacaacgaggagagcccgaaaacagtcatggtcagcccgaaaaaggtgcagagccgccccgacgggtcgatgtgctcccaacgctgcttggacaggtacacgacgatgacgaccggggtggtgagaagctgcccgaccaggtcggtgagcgccagccagccgatgcacagcagaaggacttcttgcgcttgctctcccggcgccggtagctgcgcgacacgagcagcatggccagtgcgttgcccacgaaaccagtgagcagcatggtgatcgggaaggccacggacaccgatccgcaatcctcgccagaccctggagggcgcgtgaggttgccccgcgcctcggggaacgctcgggcgcccacatgcctgtgtaggagtggttgaggcgggtgcagaagggggcatcccctccgtagccccgggtctccttcatgttggcttcgaggtgaggaggggatggcgtccagagagccgcagcgggagggggcagacgcggcgcgggcggcggcggaggtggcgtttaccgcggctggggctgggctgccccccatggtgcggggcgcag

SEQ ID NO: 14 - DNA, 235 bp, Homo sapiens
  source 1..235: mol_type = genomic DNA
attcttaattcccacacctcttcccaagttacgccaccggtcgaggacggcaggagacccccgagtgcagagaaagctcaaaccggcagcgaagtcggtcctagccaagctgaaaaaacgtctcggattcgcggacagcggcctagacacagcccgatcttccagtcctagtgccctggtcgagacggttctatccttttgcaaagaagccggaaagagctgggtcccgggggcg

SEQ ID NO: 15 - DNA, 1209 bp, Homo sapiens
  source 1..1209: mol_type = genomic DNA
cggggtgggcttcccgcgcctgcagcccggaggcgtcgagcgcctgatggagcgtcagaggagacctcgtcccagcgttgggccttctgttgtatttctgccagaaactcgcgttgaggagaatttgtttaagcgtttctctacctgtccatgcatctcgtaaatacagtaagcatggcaagccctttcgctggttaaccccggcgtccgtgtgctcactgccgccctgcacgcacgaggtcgcggatggttcaaagcacccgcggtggcagccggagaggcagagcgggctctagggactaagacagggccgagctggacgactggtggcagggcggccggggaggccccgccgcggggaggtttgggccccctaccctgcggaactggaggcaagtgcggactccggctgcaggccgcggtctgcggggacgcgcgcggggggcctgcgtcccgccacctccgcgcacacccctggtccccttccccagcccgggccggcgccgcccgccgcgtgactcgcgcgttccgggccgagtacgggctgtcccttggcccagcctcagccagacgcgggggagagagagcaggggctgggaagtgccggcaggaaaggaattggccaggggaaagaggaggaggagatagatggagtcatcggttacggagcgcgctccgtccggcggagcgaagccggggaccgagagcgcgcaaggcgaagacacagcgacgcagccccgtccccggcctccggaccgcctgccccgctcggccccggctcggctccgccccgcccctgggtccgaacggaggaggaggaggacacgagccgcgcgggcttggaacccacgaagacccgcaaacccagggcgccgccgtctccgaccacctgctgccgcggcaggccatcagcagaatttatacccagggcgcgagcccgccacggctgggcggaggcggaggcggaggcggggagggggggggggtccggcttgcgaggccccgccccgctccctgccgccccgcccccctccgccccgccggcctcactcggggcgccgggacccccactccctccggggtcggctgcccgccgccgggttgggggatgtgccgaagggcgggcctccctgcggaaagcgacgtcacggccgctcctcagtgtgaatgaatcaaaacgcccgggtgaccgcggcctcccgggcggcc

SEQ ID NO: 16 - DNA, 313 bp, Homo sapiens
  source 1..313: mol_type = genomic DNA
aaaccactctgaaagaactgtgagacctttgcactaagaccattagggattctacgcaaggttctctctgctccttgcaaaattacaggcgtcatgaaggatggcggctcgtaaatcgaagcctttgaacaggggctcaccgtccggcggcatcaacaccttgttgttctgtgtgcaccaccccacggggtgcaaatccgcgatgactacgtcacaccagaagtcggccctgcggtcctccccgtaaccgcagtagcgcagaagcagcagctgcccgcacgtggtaatgatcgtggccaccagtacgtgtccg

SEQ ID NO: 17 - DNA, 1209 bp, Homo sapiens
  source 1..1209: mol_type = genomic DNA
cggcaaccgagccagatccaagaggggtcggcctcgtctaaattggtttcccaccaatggcctcggatcagccgcggctgtgctgcgggagccctcaggacgcggctggggttggtgcgcggggcccggaaccccaaacccggctcggttcggcaaggttcagggagacaaggtagagaaggctggggtgagcgagaagtcgggcggccgatcgtcagggccacgagcctcgccttgccttcttggaatcccacccaactttaaaggcccaaagatcctgaaaattccgaaagcgaaacgcgagctggtctccagaagtttgagaacggtctcccaggctttccagcgtcgtcccgggattctcggacaccacaaacgccatcaaccacgagcaccggtgtccgtggctattgccccgaatggtccccatccgcgtccccgggaactccctcggcttttcgcgcatccaggccccagccccagctactggtgcgccccgagcccctaggtgccagagcggtggtcggccgggctcctgcccagtctcggctcctccctcctccccaccagaaggaaaaacttgggcccttcgagaaccctgtggaatgttctttgtaatcaactgtacatccgcttccacggccggcctcgtgcaaaatcgcgggtttcggggccttggagcaaattgcgcttgtcagcggcgacgtcaggaggacaaggggaggggttcgcggctgaaactgcagcttcgcagcacagagccattttaggctgctccccacctcgcggggcccatgggaaagccggccccgggaggcgcgcccaaacgcaggctggaggagccacggacgcgtcctggccggcgtgtcgccatcgttcagcctcgctgcccaggtgggaggggtcacctgccgcggggtctccaagccagtgccgcttgctcccggcccccacccactgacagcacggcgtccgagtgaccctgtcagcctcgttctgcgctcctgcaaaccacgttgctgcgctaactacaaacctggccaacatgtctttgtaaccctatcatttaaaaacgcttccaggcacctggccgctgccagatcaggttcgcgggcccggaggaggtcctcccacctgcccccgccagccccggggaccggcgcggcctccgtgtggcccccgcccacgaggtccctcgggcaggaaccg

SEQ ID NO: 18 - DNA, 630 bp, Homo sapiens
  source 1..630: mol_type = genomic DNA
cggaaaaactcgaatgtcgccctgccgttcctggggttggtgacaggtctggtcatcgcacggcggcagctcctcacctggatttagaagagctggcgtccccgcccgcccaagcctttaaactctctctgccagaacccgccaactctccagggtacaaagtacagcagggacgcgggtggagcccttccaagcggcgcagccttatctttcccgagtgaacacctaggtggattcccaacaccgcgcctggcacgtttctggagggagtctcaagctcctccagagctcccagctgccgtcctcgtttctgcagtcgatattcctgtgggagacacggggggctctgagcgctacgagctttattaagagatttgcgaatggttcactcaggtccctgaacactcccaatagcctaagctgcctgctgtgttatagcgcagaagcccctaacgcacggtggttgtccttcttctcataacgctcgcagcttagggccagtttccgcgattctaagagtaattgcgtgggcacctgtgctggggccaggcgcaaagaagggagttggtctgcgcgaagatcgtcaacctgctaacagaccgcacatgcactttgcaccgaccatctacg

SEQ ID NO: 19 - DNA, 272 bp, Homo sapiens
  source 1..272: mol_type = genomic DNA
cggtaaagatcccgcatgtagtccggaatgacggcactcttgctaggctgcgggcggcggcgcagcccaaacatctgcagaagtgtcgcctcgaagtcccgcaggagctcatggctctgccctgagcgcgtcctcccgcgtggccctgaatctcggcgacttttttcttccccgtctcaggtatcaaactagcatggctcgcgcctcctagcaggacttggcataataaaacgaccatcagcattcggttaccaggaatcatggtgtctctg

SEQ ID NO: 20 - DNA, 1209 bp, Homo sapiens
  source 1..1209: mol_type = genomic DNA
cgcgaggatgggcgggctccacgtctgggggcgctgtgagcagggccagagcgaaacctcagtctgagtttacgaactgggaaggagcctgaaccccagaaacaccgttaacaaagagggcgaacgcagggagccgaggagacgtctccttggcctctggggaaggggctggaggagcgagccagctgagaagcggctttgccgggtgtccactagcggttcgccagccctctgtgcgcccgcagagtgacgcagaggccgacacagtcccctccccactcgcccctggggcgccgccgaccttgatcacccccttcgcggagggaactcccaaggcgcggcgctggccgctgggtccgcatggcccagtcctgtcgggctccgctctgctgccgccgagcttcgaaggtgtgcagtgttgagccgtgtcttgttccgtggcttcgaggggtgtgcgcgcttcctatacctcccggaacgcgcgagatagtcacacgcgccactttgagggtcaaacaccccgcatctggccacactgtacctttactaacgtggggagggggcaaaaaaatgtgtctctccttcttaccccgaggtgtcactcgcccaaacactccctacaacttcttcaagtcaaacttggcaaggttggctggctgactgcgagaggaaaaagagggcgcggagggggcgcggcgcgcggccgggtgtagaggccacggaggcgaggcgccgagcgtcccctttgtcctgtagagggagctccagccccaaatttccctgctgcctcccccggcccgcccaccccaggcccgctggagccggaatcccggctggaaaggtgcggcggtctgacacccccgcaacccccgcgccggggccttagcaggatgcctcttctagggcacgtgggaaaacccaccagggtgccaagacgcacagatgctcccaggacccgggctccccaactctcaactagagctgggcgtcacacatcatctagaggattatgtacccctaggcgaccctcttctccatatccgcctccaccgtcaccccacaccctggcatctataaagagggaaggggggcaaccgggttggaaggaggatggaatctgggcttgcacagcctcttataggtaacagtgagaaagcctaactgtgtcgatcaaagaatggggacgaagggagtgatgagaacacgacc

SEQ ID NO: 21 - DNA, 1209 bp, Homo sapiens
  source 1..1209: mol_type = genomic DNA
cacacacacacacacaaaagttgggagagaatgtgagaatgcttgtgttcgtggacgcgtgcgcacgtatggagcggcggtacctatgattatttgcgcgtggcgcctggctatgcgtgggcgaacggcagtggagaacatgtgggcgtgcatgaagtgtgcacttaaaccaaagtgtgccctccccgttgggcaggcctgtgtgttggtccaagggttgggagcgcctgagctgtgtgagggtcgctgggaagggtgtttgaggccgtgggcggtgcggagcgtgtctgtgtgcgcggggctgagctggttgtgtgaagtggtagcgggtctgccagagagcgtttacctgcgtgaatgcacaaccacacactacgggtcacgtcccacactcattctttccctaggcgggccaggctgtctgcacgcggagacccagagaggccagcggaagggcagcgcgggaaggcgctctcttctcccgcgagcgcagatctggcggaggaggcggcgactgcccggcaggctgcgccggcgagccccccagctcctctcaccgacgtgccaggcgccccgaaggcgacaggcgtctcccgccgaaaatgaaggcgtccgagccaggaaggtgggggcgggtagttaggggagacaaggagtccggagaaaaccgagaggcgggtaggctttgaacgcgccgcgccgcgggccgtgctgcgagcccggcacacgcctctcgccccagctcgcgctggggcttggcctctaggctcttcgcgatgacccgcgaaaacacccctcgcccccctcttctcaaccggaaccctggagggagaggccgcgcccggccagcagtcgctgcgagagcagcactcacctgggtctgtgcgcgccgcgatgcgccggggctcctgcgctgtcctgcgctccgctctcggcaccacctttcgtggctccaccgcccccagcccgcacccgagcgctgaggtgcaagggcaggtctgcgtgcgagccccctgcgggctggcagccaggtgcgcgagggccgcccttgccggcgctcccagcccgcgctgtctgctctcaggaacctggcctggcgcgcagtgtcagaggccccgcggcggcggcaggccgagcccacaggggcattaggccaactccccctcccgccctgcgcacgcggaattctctattattattattaaagaa

SEQ ID NO: 22 - DNA, 1209 bp, Homo sapiens
  source 1..1209: mol_type = genomic DNA
cgagcaggagcaggcgcggccccggatgtggagccaggctttcgggagagcccatttctacagggattcggcccgacgggtgccgggccgagctcgctcggcctttgttagtttcggcgccgctgattgatcgcctaatccagtggaccctgagaagaacagactgggctgccaatggcccgaccccacgccgccgcgacctgcccgagggcaccgggagcgatgggtgcggaggggctgggaggacggtcatccgggcggctggacctctccttcctcccctccgccttcgtggctcggcgccgggcctcagggaggacgcagagggggcgcccgtggcggtgggcgcccagcggtacctttgggtcggggggaggtaaggctttctcagctcgggttcggaaccagaaagccaccggccccccgggggtaaggggtgggcgccggcgacccgggccgacgccgccacctcagagggcgcgcgcgtcccaccaaggtcggccgccggcccttcggacggacgagggaaaagccagccgcggctccagggtgcccgagactcaatcgcaccacctgcaccccggccacctccgcctccgaagtctcaggaggaaagccccgacccagcccgcagcctctctcgcgcgttcaggacccgaggctgcgggcagccgccttccacgacggacagcacggagtcccgccgggccccgagccacgccagtctcggacttcagctgggtccgaggctcctctgggcctggagggcttttagaggagaagaggaaaccagcacaggagccccagagggccggacaggtctgtgcgccagcggcccgggcgcatggcacagtgcctggatcaggattggtgcgaagccagcatcaaacggacagaaaatgaatccgatccgacactctaggaagagcatgaaccttccaatccaaccaatgtttgtgcgaggccaggttccccgccgcggtggcccacaagccagccctgtcctcgggacgcaaggtaaccagtccggctggaggccgccacgcacgcctggtctctgcagttgtggcggattcccagggactctgcccaggaaatggaaacccgtgagggcgctcctcaaagccgagggtggtgatggccagcgaacccgaaaaagggcagggtgggcgccctcccatccgcttaagcagtagcccattccactgc

SEQ ID NO: 23 - DNA, 1209 bp, Homo sapiens
  source 1..1209: mol_type = genomic DNA
cgcgcgcgcaggtccccgcccctctccccgggctgggggcggggtgggcaggccgacggctgacctggtcgaaatagatggtcatggtgcggttgctcatctcggacggctcgtggttggtgctccggtggcggagaaggccaccttggcgctgccggagcgcacggagatgcctagggaggaggtgacggcgccgtccgccgacgggctggagtcgcacaccaccaggcacttgccctccagcacgatgggctccgtgtcgttctgcgcccgcacggggcagcaggcgggcagtagcacaacagcagggccagcgccacccccaggcaggatccgcagccgcccggctcgcgcagcgccccccggcgcccgggcatcatcagccgcagcccgagtggcccccggccgggcgcctgcatcgggactggtgggaggcggcgcgcgggggtggaggccggcgccggcgcgagcgcgcggaagggcgcgaaggaacgcgcggagctcgcagcagcctccgggggccttcgtccccggctctgacgttcaaggccagggtcgttctcagaagaaaggcgcctgtgaacctgtcagggcacagaacccaaagccttacaccgggaggtggagccaccgggaagaaggggactgggagcaggtgcctgcggctcgggggttctccccgaaggccacccccttggtcccgcgcacagccaacccgctttccgatctggcacaacaggtccggagaaacgcccgggcgcagctggcagcgcatccctgaaggctcccgagcagcgcgcggggctgcgacgaggagggggccgcgcttctgcaggtcctcgtcgctaagacgggccccgagaccctcagaaggcgtcaccgccaccccgcccattcaccagcgcggagggggctaagacgcaggcaaagaaaaacctacgaatgcttgccttctcgccttcatctcacagcatttccttggagcagaccaaggaacccttttcctatcccttgtttccctgctatcctctatttttcaatcctcgagatgtttttaaagtccatttaagtccctttggtacagattttatctgttttccacccacagaacaaatcccaggaatctccttggggcgaataagattactaggggggaaatacaccacacacacacacacacacacacacacacacacacacacacacaca

SEQ ID NO: 24 - DNA, 538 bp, Homo sapiens
  source 1..538: mol_type = genomic DNA
ggaagtatcttggcctgcacgcggtttacatcattgggagcacaggcctcgccctccgaactacgaggcccagagcccttcgcgccgagcgtttcccgcctttcccctgtctcaagtcattgcccagggccactgcgggccgttcctgcctgtccgtggagctgcggcccccgtgttcccagggtgaacgagcacgtgcgaggcggtggtgcttgtatccgggataaccaggtagggcggctgcatgcttcatagaggccagcctgtgccgaccgggagctgtaggacggtctgtgtctgggctgcttatgggtattcgcgtgcgtctgccggtgcgcggtccgagcctactcacgagagcgtgtgtgtgtcttctgtctcgtgttgctatgaggtttgcatctgtgtggctggaatagcttgtttgtgggggcccgcgcgtgacctgtgtgtgcgttactgtgtgtgtctaggcaggatagtgacgggccgtgtgactgtgacgccatcatcaggttggtgagatggcgtgaccgcg

SEQ ID NO: 25 - DNA, 450 bp, Homo sapiens
  source 1..450: mol_type = genomic DNA
cgagggaggacgacaagcggggacgttggcggaggggttaatggcggagtcgcgatccagccgagcccacaactctgggaggctggttctgcctctctcccgaagcgtcgcccggctaagcttcgagaccaggacacacaaccgccgactattccagagcccacagtcggcttcacgctggaggccggaaacgacacgtccccaaagcccgcgcgcgcggcccgccagaactgacctacccaatcaccgcaccgctgcgatcaagcggcgacacacgttgccgggtaagagtcacgcttggcaaataagcaacctgcctcatccaatcagcgtctcgcctgcccggttagttccttgtcttgcccaaccagattcttgcctcctccataccgctacttgcgtgtgccggtgcctggcccaatcagcgtcttgcgcctcttctcgctcccg

SEQ ID NO: 26 - DNA, 1209 bp, Homo sapiens
  source 1..1209: mol_type = genomic DNA
cgcttgttgcggtggatgtcgccaatggaattggacaccgtgttggggcccagcaacatgcgggtgctgcgaggccactcggtgctcacgaggtggtgctcgcccatgaggatcttgcgcacgttctcgcgccggtcacgcgtatcagcggccgccccaacaaatgcgtcttgaacacgttgccatacttctccctccgcgacgactggaagccagaaccctgcgggagccacacccgggtctctctcagggtgcacttctgcagagggcccgcgagggaggggcggcggaccccaaccggggtacagtcacctgccccctctccccaccaaacacactcacacagacgagcacgcaggttttatttttcatagcgtgcacaagaactgcgaagtagggcctagaggataataaataatgcaaggaggcggaggcccaggggcacccccaggaggctgttttttggtaatgctttcgagaggaaagaggtatcccggacagctggacccgaagcgggagtctccgcccccacccccacccccacacccccaccccgcgctcgggagcctctcggaataaatatttccaggctccgggccacgggctggcgagaccccgcggggtggccgcaggccaaagattattatagcggtaagcggtcggtgcccggaggcagcaactacagatgggggttggagtttcctccgctctccggtgtggcccgcgcagggacccggcggccctcggaggactctgcagggcggggtcacgggcccagagccgcgattttcctaatgcattttgccctggaaatacggagacggactttggttgccgccctggagtttggaacccgagcgcggggcggggcgggaccgggcagggcgacccgcgcaggtaaccagatccccggtggtggcggcgcaagccgaggatggcggggccggggccggccggactctcgaaacggctggcggagccggccggcgcgggtaccggaaaccgtggagacctcagaggggcgggggcaagggcgtcccgctcacctttgacgtcggcactagttaccccaccccacgttaacccttctcctgccggggcagcacccggcgcaaaggggaggggcgccacctgtctagccgccccgcctgccagcccagagcgtaccgactcgggctccagatctacccgctcctgcacccccgaggg

SEQ ID NO: 27 - DNA, 1209 bp, Homo sapiens
  source 1..1209: mol_type = genomic DNA
cgctcggcagaggcacgatacagcgggagagaagggcaggcccgttcacattttaatcggagcgcaccggcggccgctcctcggctgcgtcctgggctgccgctcgggctcgggactgccagatgcagctctggctgggggcggcgggcgcaagcgggcgcacccgcagctaggggtgcggggtgcacgcacacgcacgctcattaagagccatgtatttattgaatgtccgagttgggttagttcattggaaatccccgaggagggctcaatttgcccttgttttcgttgccactttccttttttcttggttcgctgaggttcctctgtgcagcgtttccgcttggccgcgtccccccaccccaccccaccccacccccgcttctctcgcctaccgggtgcactccccctcccatcccccttaactctttcagctgggttagagctgagaaagcatttgtcgccgccagcccatccaccacgcaaatccatctgagacagaaaggaaagaaaaaaaagcaccaccatgcctaagaatagagagcgagcaaacccccccaccgctaatcacacacacacacacacacacacacacacacacacacgaggaagcggtggagcagagaagggcgcggctagccgacccggttctttcgcccggctcctgctgccacagggaattcctaaagccattggggtcgaatacacttacgatgaatctatgggggaaggtcggactgattgcttttcaaatacatcgcacggctccgctgaccggcaccctccaaactcacaagggcacgcacgctacttgccgaatcccagaggagggaggagggaggaagggagggagagcgaaggagggagagagggggtggaggagccagggagcggcggcagcgagcggtccgtctcgcacgcgcgggcaccgcgctggtcctgggctgcaggtttcccagatgatggcatccgagaacttaaacaaaggggctgccgccggcgcgcaacggctgcggaaagttgcggtggcggatttccaaggagcgtggccacgaccagagctcttggcgatgcgagcccccgcttcccacccccgccgatcagagaagggggccggctggtgaagggaagaggaaactttgaaaccactggggacacactgtctataggtattagcttgaatggtacatccgtgccgcgcgctttaca

SEQ ID NO: 28 - DNA, 1109 bp, Homo sapiens
  source 1..1109: mol_type = genomic DNA
gactctcatcactaccacacccatgtgtctttcctctctccgtctccctgagacgccagtgaaccacctgggcttggaatccagagccccggaacaccttgcagtacttgcccgaagcaacgcgccacagcgggagggagacgcagacgcgaagaccggcgagaggatgtgcggtgggagtagctgcggggtctgcaagcaggggagcgcgcacaacgttaaaccttgaaaccagaaatcccgatcctatcgagacccatcagcttttcccatttgggaacgagcgcaggggaaattaatgcgtaattcaagctctgttttcccctcccctccaaaggtgaggtgggggaacgcgtctcgttagctcattacaaagctttccgtgggaaattccctcttcctaatgcgtctgtcttgcaactcctattagcatccttcgacagcggcctttgaaaatctcctttaaatttcatacctctactcgtttcccaaatggggggaagggaagagaaagccggggggtagagagaatttccttcccctttccctcccaggtagccaaagcgcaccgcagcggcttggcttcctggaggaaaagcgcccgagcggggcaaagacccgcccaggctgctcttataggcgacgcggggcgggcctggctgggggcggtgccgcgcggccccgctcgcctataaggagctgtccgccacccgggtgctgattccagctctcgcgcccgacgaggtggatttggctgtccaccgagctccggcgcctgtcgttctaattgggtttggatttgcaccgttaaggagggggaagagaaggaagaggcgggcgaggaaggcgagtccagctagcggctgttgcggggaccgtagccccagctgcagctccgaagaatcccccgccacggtttcggtggagcgtctgggcacgggatggagtgaaagagcgagtgcctctccaagcgggggtgggagggggcaggctgtgcagaggagagagacagcgagaagaagccgcggctggctactgcgaatttgggattcgattgggagggaccgctcactcgggggaaatggattctttaccacggctgaccagcg

SEQ ID NO: 29 - DNA, 1209 bp, Homo sapiens
  source 1..1209: mol_type = genomic DNA
cgtacccggctctcaaaggcacggcacctttgaattaggttgcggagtggcctgggcgaggggacccggggccatcaaaggcccgagggaaggccgtggggcgacacccgtctaatgacaagtaattaaggattcgacgccgcgcattaccagcggaacgggaggcattaattatttaaaaacgattagccttgccgtgtgtcctgcaggcgtcccatcatgttcagcgaagataaccagccacccgaggcgagccggggctcggagggcggcgggcgcgctccccagcaggcttcggggcggtggacacgggcgcacagcccaaggcaccagggaaggagaggaggggccaccaagggtccccacacaggaaggtgactacataacactgaccctgctgaacaacgaaacagatttctgcccacttgggttcgaagtctggtgactgcggcgcagatgggaaaagctttcggtcgggggtggtgggcaatttccatgtgccagttccctgtcgcatcagaccaaagacccaaagtaacccagccaggcacgggcctctcacagtgcctgctacccaggcgtttaaagaaaagaccccagagagaatgtggatctggcgtgggggcagggattgggggggggcctggtagacacctggggaaatacaactttatgaggtttcaaggggcagcagcagaactactatttttccgaggtccacgcctttccgcaggcggcggaaagacgcggctggttctgttgggtcctacaaactcgcgtcctccggaattcccgagggcaagcccggcacgcgcagcgtgctcctgcccgtggaaggggggaaatcggcgactgggttgcagggagaatgcgatgataaatggccctgcgtctgctttctgactttctctcctggacccagctcgtaaccccagaggctccccaacctgcctgccgccaagcacggctgtgcgcaaaaggccaccaccgcttcctgcagccagagctaaggttcactcgcctcttagaaaggcgcggcccctcccctgctgctgcttgatgacgatccccatcgtctcctcggcctgcctgtgtccgagaaagagttgagactggactcgaacccctgcgcgccaaggggagggcgtcaccatgctccgcctcagcctcttcaaactcccatgcaccacgcacatcattttctgctga

SEQ ID NO: 30 - DNA, 1209 bp, Homo sapiens
  source 1..1209: mol_type = genomic DNA
cgacgtccagggaccaggtgcccttcggctcccgccgacctgttgctcgaaacttgccctgagctctcgctgccgggctctgggctcccaagcctctcccccgccgccgcagcagcctcttttagggcccggaagaaatgggagccggggctggtgagaggggtaggaagagggacggtagaagtttcagacccaggcatatttgggaaggcgagtgctttacatgattccccatttcccaatcggaccagcttagcctgggcaggcagcctcgtgctgaggtgcctggagaccgcccacccctaggtgctcgcttccccccgggtctgacctgatcgaagtaaatgatgcgcgtcttgttgctcatctcggatggctcgtggttggtgctccgcaccgccgagaaggcgaccttggagttggccgcccggaccgatatccccagcggggaggaagaggagcccttggagtcgtggccgggttcgagtcgcacaccaccagacacttgccctccagcacgatgggctccgtgtcgttctgtgcccagacgggcagccccggcagcgtgaggaccagcagcacggccggcaccgcggacagcgcccggcgcccggagcccatggtgagccgtgtgggcagccgcgccggctggcgctggtgctcgcccgcgtcgcctcctaccccgggatcccggtgctcgggaagatgctagcggctaggtcgacagcgctgcaggagcgacggcggcggcggcgcgcacacttccaccaattctgtggcttgaagtcaaagtctcccctcgagctctctcgctgctctgttacctttgtcctttaaggagctcatgcagcaccctttaccctactctcctccgcccaagaatcagccctgcctggggcccctgcacccactctggttcctagacatctgaaagtcatcaaaccctcacattcacacctcaaggcaaaaaataataataataataaatctcaccccaaactcaagcaccaccagctaaaccacggagcaggaacaaaaagaggggactcaaagagaagccacaagggtggcgggtgcccagcggcgcgggtgccagtcctgtctggcttgcggcagggacgagttacagaggcagaaggtccttcccaggctgagaagacgcgaggctgtgttcatggccaggacgccagcgactcccactttcgcctg

SEQ ID NO: 31 - DNA, 1209 bp, Homo sapiens
  source 1..1209: mol_type = genomic DNA
cggtgccgggagccttgcctcccgccgccacccctggtcagctctgcgcaagaacgtcgttctgtttggcagccaggccgagacgcagcctgaatgtgagcaggaactcggagaagggaagggagagatcagaaagaaggcccgggagggacccgggaagcagtgggaggtctgcgccctggagccccgcgagagcccgccggtttggcacgggctcctcccgggccgcccggcggtccaacaaaggccggccccgacacgcacccggtcttttgtgggagagaaacacaaagaagaggaaaaacacggaggaggccaacagcaccaggacgcgggggccaaccaggaactcccggagccggggcccattagcctctgcaaatgagcactccattccccaggaaggggccccagctgcgcgcgctggtgggaaccgcagtgcctgggacccgcccaggtcgcccaccccggcgccgggcgcaggacccggacaagtcctggggacgcctccaggacgcaccagggcaagcttgggcaccgggatctaatttctagttattcctgggacggggtggggaggcataggagacacaccgagaggtactcagcatccgattggcaccagggccaagggagcccagggcgacacagacctccccgacctcccaagctactccggcgacgggaggatgttgagggaagcctgccaggtgaagaaggggccagcagcagcacagagcttccgactttgccttccaggctctagactcgcgccatgccaagacgggcccctcgactttcacccctgactcccactccagccactggaccgagcgcgcaaagaacctgagaccgcttgctctcaccgccgcaagtcggtcgcaggacagacaccagtgggcagcaacaaaaaaagaaaccgggttccgggacacgtgccggcggctggactaacctcagcggctgcaaccaaggagcgcgcacgttcgcctgctggtgtttattagctacactggcaggcgcacaactccgcgccccgactggtggccccacagcgcgcaccacacatggcctcgctgctgttggcggggtaggcccgaaggaggcatctacaaatgcccgagccctttctgatccccacccccccgctccctgcgtctccgagtgacagattctactaattgaacggttatgggtcatccttgtaac

SEQ ID NO: 32 - DNA, 1209 bp, Homo sapiens
  source 1..1209: mol_type = genomic DNA
ggccggccggccttccttccttccgccctcgccctcttcatgcctcagaaacgtggcctactctgcattcggtgtgtgcggaagcagcaatcacagaggcagccctaataccggaggcggcggcggcgcagcagggccaggtggtagctcggggctgaggatcgcggcgggggcagccgctatggggcccaagccctgacacacgtaccattcgctcaagtcggcggtacgcgccgccaccgccgccgaggaggccacctgggacttgtggccgcagtccgacgagggcgacacgagggagacggtgtagccgaatcgtagccagagctgggcggcggcgagcgcccgtgcaccttcatgtgcttacgcagcgagctggggtgcgtgtagcacttgtcgcagccccgcaccttgcacgtgtatggcttgtcgctagtgtgcacgtgcgaatgcttcttacggtcgctgctgtggcgaagcgccgctcgcagccctcgaactcgcatctgaagggcttctcgcctggcggaggcaacgcagagacattagtgcttgtgggtcgtgttcccgtcaggtgcttgccaccctcccccattcgtctctcattttctggaaaagaactacaaaatattttcagaaatcctttccacggcgcctcaggtcgagcacccctttccctcgtgcagagagcgcccccggtgccctctcttgaacgcctccatccctcccgccttcctcctctgggctcatggggagggtatggaggaggagcgacagtgactccatcttagtcgagtttccatcctcgaaaatccgatccactcgggttgtttcctccaaattttctccacttggaaccagaagcacctctgctcggaaatacattaacggaggagctcacaatatagttaacggggaagctcacatctgctcgatttaaagttgctgtttcagactaacttctctgccgctaccccgcccagccgtcatcccccccacccacccccatcctggcccaaattgtttcctaaagtaggttttgcgcaaacgccaaagcgatgaaataatttaaggatgcgcagccgatgcacattgtgtgtgcataaagtggattcgtgctgcagggagaggtattctgagcaatgattcacttcagaaggatttttacaggaatggagccccctccctctttccttctaccccctgagg

SEQ ID NO: 33 - DNA, 807 bp, Homo sapiens
  source 1..807: mol_type = genomic DNA
gaaatgttctggagcatggtgacgggaatgcagaagaaggtgatgagcaggtcactgagcgccaaggagcagataaagatgttggtgacggtgcgcatggccttgctgcgggtcaccacgtagaacacagagcattgccaaagagcgccagggcgaagatgagcacgccggtgagcacgagggccagcttggcgcgtcccggcagctctggggtgtagacgagcggtcgcagccggtacagagcgatgaactgctcccgcgtcaggttgtggtcccgcagcagccgagagaactgctccgggtaatgttaagcgcctgcattgctgtgcgctcccgggacgcggggccaccgcccgctactggctggccatccgcatctgcggggcagcgagggcttcgggggaccagccggaggccgcctcccttcctctactctggagtcagccgcgcgggagggctctaggctgcacccgggaggttcgggaaaggagagcagctcagggatcaaacccacgataaagaggcgggaagccaaagcactgggagactcgatctcagtgaccaaaaaatgttcgcggttcaaataaagttcttcccttgctcttccctaaggcgaggcgccgccacggtctggggtgctggacgccacgcgagggtcctggcgcctctggctccccgccttctggccatgcgatgcggacgccggacccgcttggggagcggtggagggtggggctgaactcggtgacgcctccctcacacccagcttcagtggcgccaacgccgcttccccgcaccctcagggcg

SEQ ID NO: 34 - DNA, 349 bp, Homo sapiens
  source 1..349: mol_type = genomic DNA
gaagatgaggtgcagaagtaagctgggacctgtgagcctcaatttcggcctcttctgcgctgagacccaagcggatcttgcttggcctgtatgcgttactgggggaaatggacgtgggcctgagcgcgcaggtgcgagggcgctgccccggggccgaccaccctgcggggacactgtagctgtcattccttcttctgcaggcgggtaggggaagcggtggccaaagtgggagtcgaccgctcagcacagtctgtctgagtgttgaccaggaaagtccaggctctttctaaatctcgccgcagacctggtgacgcattcgcatgtatttaaggcgtttgcacgcagaacg

SEQ ID NO: 35 - DNA, 457 bp, Homo sapiens
  source 1..457: mol_type = genomic DNA
ctttactcttggagaaatccctcacaacaaaggacaagggagaagcaagggggaggggcgcccagtggacttggctggaagaagtgagcgggctggggttggaagagtaactcgggctgcgggctgacgcagtcggcaaccgcggaagagcagcatctcccctgcgcctgtggatacgccagtccagggatggcgagtgctttctcctccccagcttctccctcgctcttcgaggtgactcgtgggaccctgcgtcctagtgctgggtgtgaatcggctatttcacacccagttcttcccccctccgccacacgcagtcacattcctggagctattccaagctgcctccgctaagcaccgaataagcggaccctgcctggaaacttgagcgaagctgaactgcgccgaactccaccgtccagtgacccgagccagtgtggacgcccttttaatcacg

SEQ ID NO: 36 - DNA, 677 bp, Homo sapiens
  source 1..677: mol_type = genomic DNA
cgctgtgcgagccaggatccgggcagatccgctactcgatgccggaggagctggacaaaggctccttcgtcggcaacatagccaaggaccttgggctggagccccaggagctggcggagcgcggagtcgcatcgtctccagaggtaggacgcagctttttgccctgaacccgcgaagcggcagcttggtcaccgcgggcaggatagaccgggaggagctctgcgctcagagcccactgtgtgtggtgaactttaacatcttggttgagaacaaaatgaaaatttatggagtagaagtagaataatcgatattaatgataacttcccgcgtttccgggatgaagagttaaaagtaaaagttaatgaaaatgcggctgcagggacacggttagtgcttcccttcgcgcgggatgcggatgtgggtgtgaactctctccggagttaccagctcagctccaatctgcacttctcttggatgtggtaagcggaactgatggacaaaagtatccggagctggtgttggaacagcccctagaccgcgagaaagagactgttcacgacctcctcctcacagctttagatggcggagacccggtactctccggcaccacgcacatccgtgttacggtcctcgacgcaaacgaaatgcgcccctgttcaccccatccgagtacagcg

SEQ ID NO: 37 - DNA, 415 bp, Homo sapiens
  source 1..415: mol_type = genomic DNA
cgaatcgggaattggcattccgtaagggagcggagggcctcaggccactaggcagctccgaacgcggggaagcgggagcaaagatgagctcagagcgagggttgccggcgccaccctgggctgcgcatgaggggccgtggacccgccccgagactcagtgacagtcgcagcttaaccccgttgggggcgccgccccgctgaggtggttgcgtctccaagtcgtgagcctccaatagctgctcccgctttcgcgtcgcaaccccaggaccccgggaaattaccactgtgctcgtctgggcggagacccggtgcttcggaagcctggggcacatgcctgggtcccaccctagactcaccgcctctgggcccgagacatgggaatctgcccttgaatatctgggcgcctttgctgtta

SEQ ID NO: 38 - DNA, 639 bp, Homo sapiens
  source 1..639: mol_type = genomic DNA
cgctccacaacccggaaggagctcaggccggacttggcgtgagggtccctgcggggctgggtcacctacccgtccgcgatcaccccacaggggctgttagtaatctccctgcttcctaggccgttgaaagcactcaggtcacccactctcggtgaccagagcggtcagtctttccccgccatgaaatgtgagttcgggtggcagccgccgcgcccctgcagtgtcccctgcacatctccggtctcggtctcgcctttctcagctccgcgccgccttttcctgggcgccgcaggactcatgaagtgaggcggggccacagatttaagtttattccccgcaactgtcggggcggtcccggagctggtgaggacgcgctacggtgggtgcagaaaggagatccctggactctgtagaacctgggcccgcggtgtggaccgatggtggattctccgcgacaacagccctggaggggagagtgggccgggggaggggccacggggcccctgaggcctgcggggctggccgcactggccccgggcctggcggggtggtggagtccgaagcggcgaggcgaggaccccggggcccgctccccccagtcgccctcccgggatgtggccccgctgcagtagcactacg

SEQ ID NO: 39 - DNA, 888 bp, Homo sapiens
  source 1..888: mol_type = genomic DNA
cgggctccggtgcgggacaggcagccggagggagctccagacccctcccctcgcgcccgcggccgctgcgcggaggtctgcggagggcgcctggctcggtcggtcgctccttccttggcgggccctcccgacccacggtgctcagccagccccattcttggcattcaccgcgtgccttaattgtatggacatttaaatcaaggtccgctgtgaacacggagagagaggcctttctcctgaggaaggaaaggaggaaggaaggaaggaaaggtgaaagaaaggaagaggggtgggtagaaatggaataagaaaaccaggaaaaagaaataaaaagcggcgcgtgtgcgtgcgcactgacagcggggagagggatgggggtggggaacgccggaggaagggaccacagcatcctccccgccgcagctcccccaatcacacagacaatgagataacagcgacgtcttccaaaggtctttgttctcccccatcctttcgcatccaggctttttcctgcaaagcggagggggtggagggatgggggtgtgggtggaagtgggagacggaggggtgcctcccccgtgttaattaccccggctcccctcgcccctttccccgcgcctcgcctcccctgcagctccagacatgaaaaaacaacatcgaccccacctcgccccagcgcagcaacccacccacccatgtcgccctctgcttcgcccaggaaactgaaggggactaggaggaggaagaagagagcgaaggacaaggagagcagcggggactcggcagcggcagccggggcagggcgcgcggccgcccctctttacctccatcgctgagtgggggcgcagccgggccgggcgtgccgcaggggcgagttgccgcggtccg

SEQ ID NO: 40 - DNA, 1209 bp, Homo sapiens
  source 1..1209: mol_type = genomic DNA
cgcgcgcgcgcgcacccgcacgcagtctgcaatgccctcaccagtccccgcagctgggcaacccctccaagaccctgtgttacgcttaaattcctctccctcctatcgtacccaccatccatccttaagcagggaggccgagactcacagaattcccttctccaccaactcgcactcttggtctagcacgcggaaaccgaacctttctacgcggcccgaccttgcgattgctccagcatgagtggcattgacttttccaatgtaatgacttatgaaagatataatcatgtgttaaattgatcctccgtttaactgttaatggtgtgctctgggcacatttacgtatttgatgctatggtaactaattaccactagaaaaaaggataatacacttcccagactttgaaaaataatcatgagttcacttcccggctcgcagcacatcgtggcggccctgccgggaggagcgagaaacgagtggaaacccgaaagtgtgctcatggaatgcaggaggtaatgaatcctggtcaggtgagactacttagtcaaacctttaatgaaaacgtgattcacatggaaagcaggagtgggagggaacaactacacaaaggtgcctttaaacaaaattaaatggcaaaacctgctatcagaaacatgaataggtttctttcaattaaaatgcaggtaagactaaattatccacgtgccaagcaactttacatcaattaacatttttttgttgtgtgggcaaagtaaatgagcccggctcctgtgctctctgtattggcatatacaacttgaaagtctctggatttccaagccaatgagctgcaggtttatttaaattcggccttttaaatcaaactttagacacagtaggtggaaggagggaaagagaaggaaaaacaaggagtcagtaacttcagtttaacagtagaaacaactcttacggtgagagtcttttttgcccggcaaatctctggctctctgatcgactggtgtttagaaccaaggttccagtaaacagggctgggtaggataacgaggagagtgtttgtgctgggacctggcaggactggaggaggaaaggagccgccttccagcaggtccagcatccctaggttgcaaacacaccaaatgacgtccacgcagcactcgccccaccgcgctgtttttgcttgcgcagtgctcccgaagggattcgggatc

SEQ ID NO: 41 - DNA, 1209 bp, Homo sapiens
  source 1..1209: mol_type = genomic DNA
cgtcgggagcgcgcggggacacacacgccactcactcaaacccgcaattagccctttgagccccaggcacaggaaccccctccctagaactggagaagacttggaggcggcggggcggccaggtgaaagcggacaagggtgccggtagggggaggggcagaggagcgcggggacgccccgaatgcggcccggggccgggtccaagggcagcggggctgcggggcgctgcacccggcgccgccttacctggagtcaatgtccgtctttggcggagagctgcgggaggacgctttttattcgctcggtgggaacttgggagagccctgcgtgcagctcgcattccgggcacggcgcggggactgcaggcgtgggggtacctggctcctagcagcctggctcatactcagccgtaaagtccccttcgctggtcccgaggacaggcatgaatcccggctccggaaggcggtcactctccctctgcctcccggctctctcgctctcgctctcgctgttgctcgctcgctctctccctctctcgcgggttcgctcgcctgcgctctcctcctccccgcgcgccagatcagtttgcagccgtggggtccgggagcgaggcggctggcgaatggagagggaagtccaagtggccgcggtctccgggcggggagcggcggaggccgggctacgccgggggagggacttagccggcgacggccgaaaatgaatgggggagcggcggccggggggcggggagggaggagctgaacgccggggttgcttccgaggagtgttttgtacatctttaggcggaggaggagagaagcgagaggaggagagacgggaggccgcctcgagggaagggcggcccgggtgggctggggctgagccgtagggagtttcgtgggtctggggcgtgtagggtgttgtgtctatgcgcacgagcatccaaagggaccgcgcatacgcgcaaacccggaatcctggtgccctaagcgtgactgtactgtgtgtgtcgcgagtacctgagtgtttctggggcggcaggtgtttccacggccgcgtggatccatcgagtgtgggttttgtctcgggagttgcccgtgtgggtccgtttgtgtctgcgcgtctctctgtgagtgtttgacactgtttgtttctctcgtgtgtaggcgagagcccacgtaacctcccagaatgaagcaagagcttctc

SEQ ID NO: 42 - DNA, 340 bp, Homo sapiens
  source 1..340: mol_type = genomic DNA
gcgtgtcgcagctgtgcacgctgattggtcctctgctggccaatcaccactgcacttcatgacggctgtagttttcaaaatcccaactgggctggtggagggtcgcaggtgtcaggttgagacggaccgagtcgctatagtaaggagctccgttcatttccttggcctctcctggtctaggggtgcgtcgatggtctttgctgtctgtcctcgatctcggtgtgaggtaggcgcctttgggcaagtactgctggctccagcgtccggctctgtcgcctgccagtgggtccttgggcgtcctccagccccttgctgaattctgttgctgggatgcgcggcg

SEQ ID NO: 43 - DNA, 214 bp, Homo sapiens
  source 1..214: mol_type = genomic DNA
cgggcgcgggagctacgccaaggttctcctggtgcggttgaagaagaatgaccaaatttacgccatgaaagtggtgaagaaagagctggtgcatgatgacgaggtaggtgccgcttctcatggggccgggggcccgggaacgcgctgccctggggcctcctccgggctttagcggaattaatccatgcacgagagacctagcctcacgttgacg

SEQ ID NO: 44 - DNA, 420 bp, Homo sapiens
  source 1..420: mol_type = genomic DNA
cgaggagtcggtgtgaggccgcgtgcgtctctgtcgtggacacgcgtgattgaccctttaactgtatccttaaccaccgcatatgcatgccaggctgggcacggctccgagggcggccagggacagagcttgcgccgagaccgcagagggaagcgtcagcgggcgctgctgggagcagaacagtccctcacacctgggcccgggcaggccagcttcgtgctggaggaacttgctgctgtgcctgcgtcgcggcggatccgcgggaccctgccgagggggctgtcatgcggtttccaaggtgcacattttccacggaaacagaactcgatgcactgacctgctccgccaggaaagtgagcgtgtagcgtcctgaggaataaaatgttccgatgatgtggagctcctctgatgccttttttc

SEQ ID NO: 45 - DNA, 413 bp, Homo sapiens
  source 1..413: mol_type = genomic DNA
cggacgttaagccgcgtccagggctccgtgcaggcgctgtactggttgccatcgtgcatcacgatgcagtagaagccgcggctgggctggatctggatgctgcccttgcggcttgcggcttcgtgtgcagcccgatcacccactgggtcttctccgccaccaccacctcccagtagtggacgccactactgaaggcttcagaacccagcaccgacacctccacatcgaagcgctttggcgagtcctgcagtggctgtgggtgcagttgccgtaagccacaatggtgcagtcgtccgacaggatcaggcgctggtgggctgtgcccgggtccagggttagggcggctggcactgtgggggttggaggagggagagaagatgagtggggagaaggctgtggcccaccatgcagtg

SEQ ID NO: 46 - DNA, 719 bp, Homo sapiens
  source 1..719: mol_type = genomic DNA
cgcgccggctttgccgctttccctgagggtgtccagttttgtgaataaaattagagaaatctgcgggcgtagttccccaagcccagctcaggcttccgtttgcagcccggcgctggccgagctcgaacgctgaaaagggccgcgtgcaatcgttccgctgtgcctcaggacaaactccgcttcaagccagtgccgctctccggcagtttcctgattgacttaattagatccgcataatcgctggagggcttttctttcccagaccgagaacagctcatttatggggaccaaaaggaagagagggagagacgacccggacgccactgatagcaacgtactcttcgcaaaaaacactcagtaactagtggaaaagcccaacaggcatccccctccgcccgcaacgcttcctcccaattgttctgggctgcacgagggagatggccgggccgtggaacagatgcagaaggaggcccggcacgaccagagacgctgtaaccaagacccgcagagcctgtggcttcccgctctttggcggtgcagcaggcatggtgctggagcccaaggcttctgggaaagagtccaggttttcgcggagggagcgacggactcacgcgggatcgcaggcactgctgcaagacgccgaacaggctgagccggtaggcgaaagaagtagggtcacagaagaagcaaagaaaacgaaataaattccgaatcccgcg

SEQ ID NO: 47 - DNA, 999 bp, Homo sapiens
  source 1..999: mol_type = genomic DNA
cgtccgggagcctccgcaactcgcatttcccagtcgcaaccctgggtcattcctgcctccgtcccagctcccggacaattcccaaagccaaaggcgtcgagcgtgttactggctcccaccagcccaattaataatcagcttaactcgtggaggctttgagccccagtgctccgttccctgaagcctcccagtgcggagcccggctcctgcgaggacatccttggaggggggccggggcgcagggacggatttcctgggaaaaatgtcgctggcgtgttgcacctagagggctgtgggcaaggaggcacgcaccaatgtcccagttctgggctctcagatcgggattcctccaaaatcgcgacagccgctcaggtgctgggtgctcacgttggcagcacaagcgccaaatgcacgaccccgcgcgccgcgcgcgacctccacgcacacttagagccgaggtgacaagggaaggcggcgggggtggagccgctctctggtggagcatgatcaatcactttaaagggttcaaaagttcaagttcccctttggaggggagaggtaatgcttcccgcggcctgcttctctctgagacccgcgggttcccggcagccgcgcaaccactggggatctcggctgccgcttgcgggagccccttccggggaacacgctctcgggccgcgcagccgggcgaggttgacgcagctgctagggggcagtttggcttccccgggagtaccggagtcgctcggcaaaaacgtgatctcagcctgcagggcccggggcgcgcgcttttccctttctcagcaacgggaaaggcctgttggcagaggccggcggagtgctggcggcctggcggctggaggcttcggcggaaacctgagctccctgcgacctgttctgctagcaggggcgggggagagccccaataagccgccccagaggcgaccgtgcaaattcctaagaaaaagccctctagaaaacaccaagcccttccaaaag

SEQ ID NO: 48 - DNA, 1226 bp, Homo sapiens
  source 1..1226: mol_type = genomic DNA
cgtcggcgctaagcagctctggaaacgggcagacccagctgtgcagcgatgtccagtgtcgccgcatctgccccgcggggtgcagcatgagtcttcctttgtggcgtgcggctccatcggaacgcgcttgcgacgacaaattccttttttcccccccgcagttaacagttctggggcagaggctggtggagaggtccagagcccactcagaccgagatgaagatgaggaaaagcatgagcaggaagaggctggcggctgcggcgccacagggaagagctcggtgcgcggcgcagcctcccgcgaccgcgactcctgggctgcgtcgaggagccgcgttgccataggaaccgtagcggcgccccagtgaaaccctgcgttcggacaggagaagctaaccgggccgcccactcccacccgcgcttcctccccgcccccaccggccgtgcgcgaaaagcagagatccgagaaccgcgtgcggtacaaacggcaaaagcttcgcgcgcattttccgggagttgagcgcgcggccgcaggcgggaacctaccgctctcaggctcccagcccgggcgctacgaccctgtgggcgccgcctgtgcagcccctccttccagcccgctcgggcgcatcccccaggccgggccagcgacgcgggcaccgggagcccctcccccggtccgggctttggcccacacccggggaccgcggagtgggaaaggaaccaaagcgcggcgcctggccgaccgcggacgaaattcgaggccggagggcgttttcttttttgcaaaattgccccaaagccagggcccatgtacctactgtctcctttgccccacatgctccaagaaaataagacacattctaccccgagtcctaattattgggccatttccttaacgcgcggtctgtccccgtgggcagaaacatactgcgagatgcagtttggtaattaacaaagagacgagacctaactgggcttccgaaatgctggatactgcggccgggtcgccccgcattcgggcatcgcgcggttcccggccttcgggacgttccggcccggccggactttgaccgctggcgaattaggagaaacgcagaaggcggacgctccccaatttccccatcgagccttctcctcccgagtctgcgaagcccctggctcaggagacaccggctccgcgcctgggcctgcaaatccgcttccagcgagcgcaggcctgtcgctcc

SEQ ID NO: 49 - DNA, 332 bp, Homo sapiens
  source 1..332: mol_type = genomic DNA
gttacttcccaaagggcgtgttctccaagaactgcatttagcatttcagatcagagtgacccgaactcaataggattacgacaaaaactagtgagattcagataacaccgttacatcgaaagttttatccgtgacagaggaaatattgcttacgcccttaggtaatttcaaatgcacgtatgaaggccgtgcgggtttctgcaggcgaagcaaaagaaaggtggggccggcagggtcgtggggcgggggtggggcatgacacctatcccagacgcaactcgacctcgtttgcaagaaattctgtatccaactctcttcagtgcgtttcgccg

SEQ ID NO: 50 - DNA, 1226 bp, Homo sapiens
  source 1..1226: mol_type = genomic DNA
gttcagtggtttatagaaaatttctttctctctctcaggtccactaagaccgagagagagagagaagtcgactctggcacacccgggcgaggggctgccgggattcgggagctggcgcggttgatttttccgagaatcctccacttggggtgacgtcgggcagcgcgcgcgggccgtgaggttaatgcccaggcttttctctaaagcgtccgggaatgatccggcgaataaaacgggtgtctgcaaagttaatgaattgtacaggaggctgagggtggggacttcgacccggggagccagaggcggttctggtggacgcttccccgtgcgcctaggggtgcgctgggctttcccagccgaggtctgcagagcgcaccgcgtgagcttcatcgatctcatcttcttccaagtgggaggaggtggagatgctgggagagttgtggggagttgggagtgggttggagggggactgtctagattctactctggaggatccaatcacgtggtgctgttcccctgcctagaacttcaaggtttccaacggtttgaataaaacccaaacgatgcccgatgtccaacgaacgaatgatctagcctcggcctacttctcaggacttagctttgtccctgcccgcgacttcccgctgggaacgtcgtccccgccaccaccgctctgcttaggttcgaactctgggccactcttcctactccctctggtttccagcaggagaagcgcttcggaggagggggctaggggcgctcaacctcccgggttagaggaaatgcaagttgggccaatttcgccctccagactccgaggcggataggcccagggggcagcggccttgcccggcggccactgccctcctcctcccccgcgccccctggctaggctttgaagagctcattttaagaacttttccattttccttttggaggcgtgttgttcttctcagtcacctcgctttttttttttttatgagcttcagcgctgatctgcacgttcttccctccgtgtaatttcaatttgaactctccccagagcggccttttccctcttgttgcctgccaagtcgtttaaggctaagcaaacattcttcctccctcccgtccgcgcgagtctcggtctacacccgcgaggggaggcggaggcggttactccgagagttccggtgggttggagcctttcagcagccgccttcagactgtttgggagccgaaaagcg

SEQ ID NO: 51 - DNA, 628 bp, Homo sapiens
  source 1..628: mol_type = genomic DNA
ctttgccgtcatcgcgggtgaatgcaccactaccttttggcagttggaggggagacccatcagagccggagaacctgcagccacgacggcgaggatgtgcagtggtctctggggaaacgccgccttcgccctcacccggcgcgtgtggccctgtgccgtccgctcgccgcctctctctcgctgctccgcattcccgtgccctcttactttcctctctgggctcaagatcgctacgctttatttctgcgtgagcttatttgaaaccttacttggtgtcccggcttactctccacgtctgctccagaccacggtctgcccacgcacccctcagcctgcaccgcccacgtccggcccaggtgtggtcgcagccaagccccagccgcgggcccctgcgccctggctctctcccttgggttcaggccagagatgcttgccccaagacctcgccccttgccttcttgggccacctgcagaacacccttggttgtcaaggacgtgagacagccgaggacggggaggccccagggctgttcggggaccttaagtgggtccaacgggaaggacgcgggcctgcgagggatgccggtcgggcctggctggagccaggcgcgcaggtggcgcatcttcg

SEQ ID NO: 52 - DNA, 1226 bp, Homo sapiens
  source 1..1226: mol_type = genomic DNA
cgggcggacgaatggacggattcgagcgaagcttatttcccttctcctaacgcatctacctataaatagcgctcaccgcgctgagtcaccaaaaccccgagtttccccaaatagcgcgtaacgactgtgggatcgcgtccctcggggctctgtgacagactccaaccacacacccgctgggctggactgcgggcccacacggcccagcgggtctctcaccacccggcgccccagggacgcgggcgaggccgcggtgtccggacgagtcgacagcgtcggacttcccgcgcgcggacggcgctctggggaaggaggagccggcgggcggctggacctggccttgactgcgcggggtccgcccccgccccctcgggagcgcagagttgcggggggccggcacagcctccgccctgcgccgcccgctcacacaatgggacgcgtcgggagggggcggccgcggctcccattccggtcccagggcggggactccgggcggcgggagaagcagtcgggctggggccagaaggaaaaccccctctccctctgcccactcaaactgcgaaagggctccagccggcggtgccaggggctggggagcagctggggagggagcggaagtgccaccccctgcagtcaagggtggtgacgtcacccctgcagccagcctgtaggggggtgtctgtacatcacatgacccggcccccctctccccgccgaagcccgcgcgcgcgctctctcccccagccccgactcccacctctcccccgccccctctccccaactttcgccctccccgctccggcaccgtgtcaacagcgccagctccgccccctccccgcgccctgtggaaccgccacggcggcgcggcgggcgctcgcgctcaggtccgaggctccgagaagcgagcgtgccccggttccaccctgccccccagctcagtgctgctggctgcgtgggcaccagagccagggggcacggcggcgtgcagcgacagcgcttctctgctccatcctcccacagcagttagaggacgaagctgaggcgccgggtccgagctcggagggagcgaggcgcatgcacggcctccgggccctgacgcgtagcctccgggggtcggcccacgggcgagagaagcagccgggagcccctcctcgaccccagcaaggcggcccgcgcctccgctctccctaccgcccgggtggcgcacgccagtccccgtctttggc

SEQ ID NO: 53 - DNA, 988 bp, Homo sapiens
  source 1..988: mol_type = genomic DNA
cggaagcctcatcccgccaagccttcgcctcctcgctgagactctgagctgcgctggggttggcgggcacccgattccgccccggcccagaccggtcactcagtgtgtgcatatgagagcggagagaagcgacctggaggccatgggtgggggcgggtggtgaagctgccgaagcctacacatacacttagctttgacacttctcgtaggttccaaagacgaagacacggtggcttcagggagacaagtcgcaagggcgactttccaagcgggagatggtgaagtctttggacgtgtagtgggtaggtgatgatccccgcagccgcctgtaggcccgcagacttcagaaaacaagggccttctgtgagcgctgtgtcctccccggaatccgcggcttaaacattctttccagctgcggggccaggatctccaccccgcgcatccgtggacacacttagggtcgcctttgttttgcgcagtgattcaagttgggtaacccttgctcaacacttgggaaatggggagaatctccccccccgcaacctcccgcaccccaggttcccaaaatctgaatctgtatcctagagtggaggcagcgtctagaaagcaaagaaacggtgtccaaagaccccggagagttgagtgagcgcagatccgtgacgcctgcggtagctagggcatccaggctagggtgtgtgtgtgcgggtcggggggcgcacagagaccgcgctggtttaggtggacccgcagtcccgcccgcatctggaacgagctgcttcgcagttccggctcccggcgccccagagagttcggggagcggtgagcctagccgccgcgcgctcatgtttattcacgcggccttgagcagccgagctccaatccatattaatcaaccgctcgacctacacaagtctaagtttacgggagaaaacctagtccccgaaaggaagaacagcaatccggacaagcagttggcgcctttgtcccg

SEQ ID NO: 54 - DNA, 603 bp, Homo sapiens
  source 1..603: mol_type = genomic DNA
cgacccagcccccgaggagaacccagaaccgacccagcccccgaggagaacccagaaccgacccagcccccgaggagatcccagaaccgacccagcccccgaggagaacccagaaccgacccagccccgaggagatcccagaaccgacccagcccccaatgagatcccacaaccgacccagcccccgaggagatcccagaaccgacccagcccccgaggagaccccagaaccgacccagcccccgaggagaccccagaaccgaccagcccccgaggagatcccagaaccgacccagcccccgaggagatcccacaaccgacccagcccccgaggagaccccagaaccgacccagcccccgaggagatcccagaaccgacccagcccccgaggagatccccaaccgacccagcccccgaggagatcccacaaccgacccagcccccgaggagaccccagaaccgacccagcccccgaggagatcccacaaccgacccagcccccgaggagaccccagaaccgacccagcccccgagagatcccacaaccgacccagcccccacggagaacccagaaccgacccagcacctgggcaacattctgc

SEQ ID NO: 55 - DNA, 902 bp, Homo sapiens
  source 1..902: mol_type = genomic DNA
tcagagaatttggaaaggcctactctgaggggagtaaaacttcaccaggccctgggctggaagcacaggaggtcgcggctgggcccagcgccctcgggaggccaagggcagggagccgacccaaggttaagccctccagctctccgtcgcgggtttgggtcccgtctcaagagtggggcgcgcgggctgggcctccggcctgacaccctctcttctctccatcagtgagcgacagctccccctaccacagccccaaggtggagagtggagcagcctgggccgcaacaacttccccgccgccgccccgcacgcggtgaacgggttggagaagggagccctggagcaggaagccaagtacggtcaggtgaggaggcgagggtcaggccaggtgggccgcgtgcggcggggatttaggcgatggaacactttgtgatgggtccctttctgagcttcccgcgagagaagcccaggctggcgtccctttgctgctacgagccagatccttcgtggactggggcgaagcagaggcctgagcttggaaggcggagctggggcctcgacccccgccaggggccgggagcgctcgtcagggcgctgggggtctggggcggcagctccccggggcgaggctctgggaagcgcctccaggcctgtcggcctccgggagcttgggaggcggctcccgaagccctttcggcggctctcgttgggggtagagttaaccaaagaaggcgcttctgaagggccgagcggagcagccctggggcctcagagccgcgccttcacgcccgcgaaacgcgcgccccggtctcggccccacggtgcgactgcggctgcggggtcctcacgttcggactttctctccggtggctctcggacaaacacgcttggccaatcctgcg

SEQ ID NO: 56 - DNA, 332 bp, Homo sapiens
  source 1..332: mol_type = genomic DNA
cgctacgaatgtaagatgaatttagtggcgcggctgcctccctgaatccgaacggtaatgatggatttatcaacggggattcgccttcagcccagcgagggaggtttactcagcaaatatgtcggcggcacccacccaaatgcaatcatttgaccccctgcggacgatagctgccgtgccccttcccaccgctggtcctgcttggccagggtcgctgggggccagaggccggcccggatgaccagagaggtggcctcacggatccttggcctctcgccccctcctctgtttcctcctctcagcttgaggctctcctggcaagggggatcgat

SEQ ID NO: 57 - DNA, 527 bp, Homo sapiens
  source 1..527: mol_type = genomic DNA
cgggggggtggggcgtggagggcggcctgagtgccgcgcctggcccctgccctgggtgcgcaccaggccgcgaaagccgggggccgccttctcggctgctgaagcccgggagtcagtcaagcgcccttggcaccgcgagggaggcgcacctcgcctggtgcagagttgccgcgacccgagcgggaggaggcacaggacgcagggctcctggcctggctgtgggcttggccttggcgcactaagcgctccccatctgcccgagctggcctccgcggccctctcggcccagtctacgttcgtttaaggacttcagcgcgaagaagagcgaacgtggctagaacctgttgacctctcctctggcctggaagtccgcacagctcgtggcaacactggacggacggttgtcaggggacgaccgcgggcaggtggggaggaggccttggtctggctcctggattccaccctgggacggaaggggaagcggaaaccttgacctcagccacacaaggacacctatctggctgtct

SEQ ID NO: 58 - DNA, 326 bp, Homo sapiens
  source 1..326: mol_type = genomic DNA
cgcctgctgcctgccgtggggccgctccgaggtgccaggaactccggtaagggtcccggcggcggcagcccgggagtgggcaaagagggaacatgcgtgtatttcaaagaaaccagcgcggtctctgacttccctgtcttcgctctccacccccgctccccctcaacacacacataatataacagacgcgaagataatcctgccatctggcaaatcccaaatttggtgaccaagctcggaaagcaacagcgacggctcatcccggagtaggtggctggtcctgccggactctccggagctggggcaagggccgtgggctctgcccg

SEQ ID NO: 59 - DNA, 331 bp, Homo sapiens
  source 1..331: mol_type = genomic DNA
taggacctgaaattgcagagacatagaaaatcaatacgtggtcgggagtcaactgcgcgcgaccaagggctctgccctccctctctccgttgatttggacgctgggaagctgcccgccgtccttcccaccccctttgcccagccttggcaagacctctccggaaacatactcgcgttaaggaactgggaaagcttcctggcccggagctaggagcccgacgttggggctccttgctgagtgagacgctcagtcctgtgctcccgccccgccccaggcgcccagtcccgctgggatccatctcctcagccccggtttcccgctctgcgagcg

SEQ ID NO: 60 - DNA, 1226 bp, Homo sapiens
  source 1..1226: mol_type = genomic DNA
cgcgaggatgggcgggctccacgtctgggggcgctgtgagcagggccagagcgaaacctcagtctgagtttacgaactgggaaggagcctgaaccccagaaacaccgttaacaaagagggcgaacgcagggagccgaggagacgtctccttggcctctggggaaggggctggaggagcgagccagctgagaagcggctttgccgggtgtccactagcggttcgccagccctctgtgcgcccgcagagtgacgcagaggccgaccagtcccctccccactcgcccctggggcgccgccgcaccttgatcacccccttcgcggagggaactcccaaggcgcggcgctggccgctgggtccgcatggcccagtcctgtcgggctccgctctgctgccgccgacttcgaaggtgtgcagtgttgagccgtgtcttgttccgtggcttcgaggggtgtgcgcgcttcctatacctccccggaacgcgcgagatagtcacacgcgccactttgagggtcaaacaccccgcatctggccacatgtacctttactaacgtggggagggggcaaaaaaatgtgtctctccttcttaccccgaggtgtcactcgcccaaacactccctacaacttcttcaagtcaaacttgggcaaggttggctggctgactgcgagaggaaaagagggcgcggagggggcgcggcgcgcggccgggtgtagaggccacggaggcgaggcgccgagcgtcccctttgtcctgtagagggagctccagccccaaatttccctgctgcctcccccggcccgcccaccccggcccgcctggagccggaatcccggctggaaaggtgcggcggtctgacacccccgcaacccccgcgccggggccttagcaggatgcctcttctagggcacgtgggaaaacccaccagggtgccaagacgcacagatctcccaggacccgggctccccaactctcaactagagctgggcagtcacacatcatctagaggattatgtacccctaggcgaccctcttctccatatccgcctccaccgtcaccccacaccctggcatctataaagaggaaggggggcaaccgggttggaaggaggatggaatctgggcttgcacagcctcttataggtaacagtgagaaagcctcaactgtgtcgatcaaagaatggggacgaagggagtgatgagaacacgaccccgccaccgcgcaggcta

SEQ ID NO: 61 - DNA, 254 bp, Homo sapiens
  source 1..254: mol_type = genomic DNA
cgtgtgacactttggaacgcttctctcgttacaaaccagcgtctttactgaaggtccttccgctgcagagctgcgcaatggatgtgtggacagagctagtcggcacgtggaggctctcctggttttcagcaggaaacgtgctgtgagcaggaatcgcgagtgagttccatggcgtccccgggtcagcgcgcgtgcaggaatcgcgagtgagttccatggcgtccccgggtcagcgcgcgtggggtcctgtctcg

SEQ ID NO: 62 - DNA, 283 bp, Homo sapiens
  source 1..283: mol_type = genomic DNA
cggcggggaatcgggtggccacgcacgtcggcctaaggggccctggcagcgcccggcaggacagccgtccactgcacacctcagagccggcacccgggacggtcagcgtccccatgggcccggcaccgcagctgcccagatcctcacccgcacacaggaagggtccctgcaacccagcgtccaccgtggagaaacatgaacgtgagcaaaaatacggcctgcggtgggtgtgcgaccccacacagaagggcgagaggctcgtggccatcccacacactgggag

SEQ ID NO: 63 - DNA, 947 bp, Homo sapiens
  source 1..947: mol_type = genomic DNA
tttcgctagatcatttgttaattgtgggaaggaaggacgcatccggggagaggagatccagggctggaaaagtgggtgagcggcgctggcgcaggcggccaggagagagcagatgaaccgttcgttagagcagcgaggtagtcgtgagcgctgagatccagagactaggacccactccctctctgagcagcaaattgggaagaagatgctcactcggtaagggcgagggagcccggcatggcgccccaccacgggctcggtcttctgcgcgccaagatcccgcttggggcgaggcgttgggtcagcgtttagagccactccctgcgctggtggctggacatagcctccctatcccacctcatcttcccccatccccgacagaggaggttgtgaatctacggcccttgacgttgaggcgtcggagggcgcacctttgtaattgcggcctcccttcgccccttaagtgccgcttctgggcgcctaggctggatatgaaagccccgttcctaatcctctgctctggtcccctcctctgactgctgggactctaagctaggccctccccaggttccatcactgcggcgccaacccgcggctgggctgtccgcaagagggagttgaaggcgcgcggaatcccgaggtgcagctgaccctcctctcaacgccgactcgccgctcccgcccggccacctccctgtcgggcagacttcctgttctcctgctcacagcagggaggcagtcgccgagccggtcagcagcgtgcacggagatcttcactctgcgcccagccccgggacacaggtgcagtctccagcggagcactgcggagtgcgcgccgtcgagcactagggaatcctagacggaggacttggtccattccacgcagtcccaggcaggtccgcagcggagggacgcagcggtctccaactcctggtcacgacttggcg

SEQ ID NO: 64 - DNA, 289 bp, Homo sapiens
  source 1..289: mol_type = genomic DNA
agaggaggcggagacctgaccccagaaagcagcactcaaacagatgcagctgcacccacgcgcccgcctgctgccagagccactctccggagcctgcagtcctgaagacgcctcagcaggggcaggcagaagacaccccgcgaacccccagggccggccgggcgcatcactgggagccggggctatctccctccgtcagcgtcttgaagtccatggagagggcctgctcttccgcctgggaaggccgcttccagtcggcccggtcaagatgtagagcaccgccccaccg

SEQ ID NO: 65 - DNA, 368 bp, Homo sapiens
  source 1..368: mol_type = genomic DNA
cttctgcagacagacaagagccctccttaccttccatgaagagtccgtacacgtaggagccctctcgcggaggagcggtcatgtcctctcggtttttcttggtcacctcgacagacagacacatctttccaggggccactcgttcttcctggccatggactgcatgatggccgtgaggaacgactgggggttgaagaagccggccagccacacggtggtgggcagggcaaagtctgtcgtccaggcctcgagttcctgcaaggcacacgagccgctaggaggagaggacatagaatgacatgggaacgaccgggccttggccgttctgagcaggggtcacgccagaaccttccacgggccactcagcg

SEQ ID NO: 66 - DNA, 265 bp, Homo sapiens
  source 1..265: mol_type = genomic DNA
cgcctcacggggcctgccagcaacgcgctgtgtgcagcaggaagcccacgccaggtcctgacggctgcatcctgtgtgctctccggcctggccgcctcaagacacgccacctttgatgcacacgcgctccggaggcacaggaaggacgggggcactgggaagtccatgtttagtctcctgacgtcgaccacacaaaccagacggaacggtgtccccggcccaggctgagcggagcacaccatgagggcagaggctacaggtgatc

SEQ ID NO: 67 - DNA, 1138 bp, Homo sapiens
  source 1..1138: mol_type = genomic DNA
cgtcggacgttcacaacgaccacgcttttccccgcttcctcgattagcctggcctcccttgctccagtggggcggactcaaatgcgcacgcacgcacacgccgccaagccggtcaaccgcagacaaactgactcttcgccaactagtagccacggagagcagcaaagggaccaaggtccttcaccgcctgctccaaaaagacacggcaacctccagcggcgccgcagtagctgcctctgtgctgcagcctccggtcggagtttaggtctaattccttaagaagtatcgctcctctgcccttcgtcctactccccttggcttcgcccacttcgcctccttctcctcgggaggccaggaggacccgggtcactttgccaattctgcggagggggaagtagcgtgagggagattcccacgcaagtagcccgaagcccactactctttgccccacctctccactctcttgcccgtgtaggtgccggtgctgctggcgaaagtcccagttcttaaggctcctgagctcggaaggggtgtgaggagccagatgggaagagaagcagcgggacgagggcgaggaagggctggagattgttagaggacagatggggcgggactggggagaggggcgaaggcgacgcgccgaggcactcctccgccccatgagcacagacgtaactccgaaacgcgcagatgggccgtaaggtaaagaggccttttttccctctcctctccgtcctctaccctgctcccctcctagacacttggtgaaaaagcaaggtgcggtgtacctggagcgaacaccatgattcttccagccgctacgcccccagattaagagagaaagacaggcagacggaggatgggaagaaggccggacttggcggggtccggtgcaggagggcgcgctgggcgggcggcgggcggggcggtcagcaccccggacgctcccgccgctagatcctggggccgcagctccagccgccgccgcgcgctctgcctccacaatgcggccacagcggcggcgggaggaggagggtgaatccccgctcagtccatccctcattcctgtgccagctcgggacgtacactgctgcttagggagcaaagcacttccaccgctttcacgcgacgaaggtcg

SEQ ID NO: 68 - DNA, 315 bp, Homo sapiens
  source 1..315: mol_type = genomic DNA
gaccacaaagcgcttcctcatcctttcctcgccctttgatgccgccggcaacgtgactctgcgagcagcggggcagacgccaggtctccctcgcaggcgggaaaggggctccaaggcgggtgctgcctgctcgggtcacatggctacgtgggggccttgctcaaattcacttcctgccttcattacaaaactgtcaaaggggatcgcacgtttgcagggtgtcacccaagcattctggttttgcaaacgacgctgtgcggcagcggtctgatacctgatgagctcggtgtggcggggtcggcagcatttcctccg

SEQ ID NO: 69 - DNA, 578 bp, Homo sapiens
  source 1..578: mol_type = genomic DNA
cggtgtctggaggaagcggcgcggacagtctcccgctccaccgtgtcgggcctgaagaggccgctgaacgctcgcgattactgaggcatctggtaggatcgcaaggccatcaacctacaggtgaagccgaggcgctgtggcgctttgaagccggagagaagctcagtagaatcgggaaggcgctggggctgtccaactccaccttggccaccattcgtgacaacaaggaaaagatctgggcgagttcccaggaggccacactgaggcggccaccaagttgacgcgtggccgcagcctggtgatggagaacatggagcagctgctgagcatgcggatggaggaccagagccagcgcaacatgcccctcagtgtggtgctcattcgggagaaagcccacagccgttcgagaaccgcaagcaggagcagggcgagggcgcctagcccgagagcttcggggccggtcgagggtgattcgctggctttaaggcgcgccacagcttgagcagcatcggggccagcggcccaagaacgcagctgcaagaatccagcgttaatgcgcagggtgatcccggagggcg

SEQ ID NO: 70 - DNA, 682 bp, Homo sapiens
  source 1..682: mol_type = genomic DNA
cggccgttcggtgctgcggtatcccatcccccgcctgtttaagagaaattgatcacgttaagcgggccctgcgtcgctgcgcagtgcccggactaccgcttggatggattgacaagggaaggacgcagacacctaagagcaacaagggtactttttaagcgaggttacatgtgtttaggactcgcgaggacgtcacgtttcgaatttggtaaaagtcctcggtagctaggacccaacagttctgccctcctggaaacccaatgaggtgacaaaactcccatctcattactgggagtccagcgcactccgccaccgcggctgcagcttctgctagctcggagcctgcggactccagccggcgcgaactccgccgcgactctttctggctctctcgctcacgccttcggggcggaaccgcgggcgccggagtggtcccagactcggctgggaccgggcagcctagaagaagggtcccctcagtagagaccaggcctccagctctccgtccggcgctccgctccacaacccgccagtcatgtgaggtccgtcaagggagcgatccctccgtctgcccgggcttccgggctgtggaccgcactgaacccagtcccgcagcaagccctgctgccgccttttcctgggcacgcctgcggtggtgacggcccgctgctatgtctccacg

SEQ ID NO: 71 - DNA, 265 bp, Homo sapiens
  source 1..265: mol_type = genomic DNA
aaacacagtgccgcagtgaaagtgggacaaacgcgcctgtgtgcatcctggccgaggtgccggaggaagcgtggtcggggcagaagtgttaatgggcctccttcctgccgtgccctgcccgtgccagtcctcggtgctcatcccggctccctgaaatgctcgcttccactcagggccagcgcactccctccacgtccctggccgcagatctgtcctgctttgacaaataacccgctcagccttcgggtttatggtttttccgtcg

SEQ ID NO: 72 - DNA, 1226 bp, Homo sapiens
  source 1..1226: mol_type = genomic DNA
cgctccctttcccgcacacgcattgctgcctcctctcgggcttggttaatgagtctgtgcgccaagccaggtcgctccgggcagttcgcgcttccgcgcctcggcactcggatgaccgtgtcctattgtctctctcgtgaatgtcgctgaaaaaaaataaaagtttgttctaagcccgtggcatttccgcgcgcagatggaaaggcaagtgggagaggagggtgcaggtgcggggcgggcgcggcggccccttcaggcgccggggctgggctgcccccggccgggagcagctccgctccgcgcacgtggctgtgggcgctgcggacgggggacggcaggcgggggacggcaggcggggagtggagggtgcagtgcccgcaggtgccgacggcttcctccagagcccttcgcgccgcgccttgcctggctggtggcgcggggaaaccatggcagcattgcggctccggcgggcgggctctgcgcggggccccaccgagctttccgggagcctctcccgcagccgatgggcatctagggcgcagaacgaagagtgggcgccgaaacgggtgtaggcgctggaggccgacggggaggcccggggcggtcaggcttctcggtagagagggccgtgcacctcgcggcctgcgggctaaggcggggagccgctcctgggcggccgggcgcgcggactggagggagggaagcgccacggactggggaaccggggtgacggggagacgggggggctggtggggtgaggtcggcgggcagcagccaagtgttctgcagctccgaaccccgccacccctgggcgaacggaatggcacccgcggagacgggcggggacgggcgaaagcgggtttgctccgacccgaagctgagcccgggttggagggcgagaccgtaggcgagaatggccgcgcgggctgggagacccgggccgtatggagtgagtggagccgcccgaggtagggtagttggggatggtagttgtgaagacagtcggagtctagctccgggcagacgaaacaccggggcgcgccagggtccgtttcctccttctctctgctttcccaccaccctgtagatacggagccagcagtcgggctagagcggaggctgagggtcgcggttccaagggcggtctctcgccccgggtcaggccccaccccgcggtggcggtcgcgtggcgctgtcctggagttgagctgtcccgcgccgcggcc

SEQ ID NO: 73 - DNA, 461 bp, Homo sapiens
  source 1..461: mol_type = genomic DNA
ctcgaatcttggagacactggcttctagtcgcacatctgcactttctcccacctgtggcctctcaggctcggccgcccgagggagtttcttttattcccagttcggctttcttttgcgaaggccgagtctgggcctgccaggggcctgcccgagtcctctatcgcgggtccacgtggccaccaatgacccgcggcgcccccgcgtgtccccgcagccactccgcggaagcagcggcgggagcgcaccaccttcacgcgttcacgctggacgtgctcgaggcgctcttcgccaagactcgctaccctgacatcttcatgcgggaggaggtggcgctcaagatcaacctgccggagtctagagtccaggtgcgcactccccgggctccagggtctgggtagggagctgaggctgctcggggaaggtctaggaaggctcttgagtagttcttgaggagaccatc

SEQ ID NO: 74 - DNA, 1226 bp, Homo sapiens
  source 1..1226: mol_type = genomic DNA
cggtttggagaggggagcgcaaagcgctggacgcatgcggtacagtgccacggccgccggtgggctccactgccctggggagctgaggcgcgacgaatgaagcaccagggcgcctggtgggcgccagtctccggtctggagcctgctggcctgtccctccggggcgctgaacccctagtgcggcgtcctgggggccgggcaggaaggatggcctctccacctgtctgcgaatgcagcccagccagtttgagcctccgcagaggtgcgctccgggactgggcgcttctcgtgctgtgagaacgctgggccttgttagctcattaacccctctgtctctagggcccgttggcggcacggtttattttattttacctgttttcctcggagggcgcgaagactccacccgcgcggggacctgggatcgacgactttgatactaggcggtatcccggagggctaagtcggcggaaatccacttgaccttgtagcgttagtcctttcttttcctttcctttcctttttctttcttctctcttcctatttatttatttattttaaaaataggattaagacaccagtagaagctgtaatcccgttccttcccccactcccagtcttccgggccgcctggaggtccctgccggagagagccactttggcgggcgcaagcccctgggcgccctctcctgaccgccgcgctctcggctcggcttggctgttccgggctcccagaggctaggggaagcgaggggcgccaggggcttcccggcctcagcgtggggcgaggtcccggctgcgaccccggagagagggagaggggggccatgatgctaggcgtcgaggcgagggttgagcctgtttggcttcagagaacgatgcgggttcgaccggaagcggggcgcgtgttctaggggcccttgggtggattagggggcgccaagagggttaggcgaggagaggcctggccacccagttgctagactttaggccgcactccaggaaggagcagtcggcttccttcctaggccgcttctattgcgcgccactccttgaatctaagcattttccactccaagaaccgttgggagagaggggagagcggagcagagtctctaggcccccagggccgcctcccggggtggccacgcctgccggtagcgagccgagattagctaggtctgtgctcgggcctcagcggtggccctggctcttccctaagcagcggg

SEQ ID NO: 75 - DNA, 1049 bp, Homo sapiens
  source 1..1049: mol_type = genomic DNA
cgggcgcataacaggcaccccgcacatcgcaccgcggacgcagcgatcctcccatttgtaccctaaggcctgagaaggccgctccacacctccgcgtgcacagtccctagaccagtgagggcggggaggggaaatccttttgttttatattggaaaccttacgccaaaggcaggcaggaagcaaccaaaggaacaacaggccgcctgagccgtggctcaaacagcacagggcagtggccgagcaccaggcttttcggtggagcgggataccagcaggcagaagaggtggacaactctgaccgcagactccctacaaccccgcgatcgatgctccacctgcacgctcaaacgcacctgtttcccaaattaatccagacgcgaatttaacgaccgattttgtagatccgggaaattctgtccaccagcgtcacccaacacagcaacttttacagacaatgcgtggcgtccgttcctgctcacccaaagcggcctctctgggctgacagccacccctgcgggtcctcaaaccccactcaaaccctccgaacccctgtgtctgggagcagtagggtgggatgactgcaggatcccttgaagaagtgactcgctgcaaccccaccaccagcagcaaaaacgctttcacccgggagctgcatggatgcgcggatggcgggggctgaggggacgcgtgaagcccaggcctcttgcttgggctgggcgattcccgccgtgggacgggtgggattgaccttagtggagtccagtccttcggagaatccgcgggcagtcgggacccacgctccggaacacccaagtccgaactcgtacacacagcacaatggcgtgcagccgccggggccctaactcccagagacgcgtctgaagaagccatacaagggggcgcagcttcggggctctggctgggaggttactgcagggacagcgcctcgcctccagcagccgggtccaggctcagggccggccggcctctgctcgctttctgcttggattaccaacagccactgggcttggggagatcggccgggccg

SEQ ID NO: 76 - DNA, 785 bp, Homo sapiens
  source 1..785: mol_type = genomic DNA
cggcaagacgcgcaggaagaccagcgcgcgggatgcgtcccccacgcccagcacggacgccgagtaccccgccaatggcagcggcgccgaccgcatctacgacctcaacatcccggccttcgtcaagtcgcctatgtggccgagcgggaggatgagttgtccctggtgaaggggtcgcgcgtcaccgtcatggagaagtgcagcgacggttggtggcggggcagctacaacgggcagatcggctggttcccctccaactacgtttggaggaggtggacgaggcggctgcggagtccccaagcttcctgagcctgcgcaagggcgcctcgctgagcaatggccagggctcccgcgtgctgcatgtggtccagacgctgtaccccttcagctcagtcaccgggaggagctcaacttcgagaagggggagaccatggaggtgattgagaagccggagaacgaccccgagtggtggaaatgcaaaaatgcccggggccaggtgggcctcgtccccaaaaactacgtggtggtcctcagtacgggcctgccctgcaccctgcgcacgccccacagataagctacaccgggccctcgtccagcgggcgcttcgcgggcagagagtggtactacgggaacgtgacgcggcaccaggccgagtgcgccctcaacgagcgggcgtggagggcgacttcctcattagggacagcgagtcctcggtaagtgcgctgcgcccacagctccggctgcaggcagtaaatgcgccttgcgcggtgggtctgtgtgccgcg

SEQ ID NO: 77 - DNA, 1226 bp, Homo sapiens
  source 1..1226: mol_type = genomic DNA
cgactcggtgaaggatgatgcccggagggtgatctcgacctttaacattccacacacctacctccacgcaccaatcgccggaatctccaacccgcgggccgcgtgggctttctaccctgcaccgctgagccgcggccacgggaagaggcgcgctcccggcggcccaagctgggagccaagctctaacgggtgtggcgggaagtgtggtggcccgccagcagctgccacgacgctcgctccaccgacgcccagagctgtggccgggccgggggctggcacccgctgggccgccactctcggggattttggtggcaaagcggaggtcccgccgaggctggcgaggtgcgcggctggctgctaggaaagagatccagacggtcgcctggtgcgctggatcccgtgccctttccctgaaacccagcctggcctgactgcaacctctcccaacttcagtgccggatcccctagacaatcagggtgggctccccgctgcgagcgcgccccacagccgggtgccgccaaaggtctcctgctgtagcggtgactcacattcccagtgatttagaaaaactgtggtgccgagtgaaagaaaaaaaaaaaagcaaacacccttagacaaaagaaaaaagctccactctttccgcgagcgggaaaaaaggtttggttcagcagtgatgctatttcagtgaatcagacgctctggggctctgcaaggaaacgcacggactgggagaaggaagtgggagcctagagttttgctctcggcaaaacaagaactcggcttgtcctcccaggccggcggtttcctacggcaagacaaggcgcggaaaacatttttaccatctgacgctgtagtctgtcctttagaagagaataggtgaaaagtttaataaagaaattaagcgatgtggaagtcgttcccgctgcttgcagagggtggggagcgggcctgggcaatttcggaactaggagagaccacgtcggcaaagcctgtggtcccgaacaagggccatggatttcccatttctcagatcccggctgcccgacttcattcttgtttctttcagcacaagccatcccccttctccctccctccacgtctcttggcagagacagaaagggacaccgctctgcccgcgtccagcgcgaaggaggtgacctaaaacccatgggcccttctcacactgcgttctctgcgctccggcccagttccggaggacctcccg

SEQ ID NO: 78 - DNA, 242 bp, Homo sapiens
  source 1..242: mol_type = genomic DNA
cgggttggagtcgacatgggccctgacgagggcactgatcagactcaccgggggcgaagggggagagggctcctgtgggctcttcggtttcgagggcaaacgacctctccggccttttaaactgtcttgcgaaccactgcaaaggaagagccctgttagcgccgcttttccgagcccaggcccagctgctgcctcggtccctccccggggaaggccgcagccgcggggcaccaggctgagcg

SEQ ID NO: 79 - DNA, 943 bp, Homo sapiens
  source 1..943: mol_type = genomic DNA
tcctaaccaatttcattctgaacagggaagacagctcctagcacatgcaaatgaccctcttccaactccggctccagcaacttcgggcgggggccagccgggtcggctgaatgcgagggggatgcgacctggcctcccagtctttctgcttccctttctcagacacccggaagtccccgccgcagcccatggtctcctgcagggcagcttcggcggaccccggagagctgggcagtcccgggagagctggggctgggctactgcaccaaggcagagggcacactccgaggtcccgggcactaggggctccctacctgcctactccgctcccgccattgctcaccttaaagaagcctttgcagccctcacaggtgcgcacgccgtagtgttggcaggccggttgtccccacacacagcgcacagcccctcgttggagggggagccccgcgacggcggtgagggcacctgcgtgtcgagcagctgagacgcgtggccgatctgcaggcccgggaagcccatggacgcgggcttgcgatggggttgggcacagcgaaggtctgcccgtccaccacgtggtggctgccggcgggctccgggttcatggggacgtgcaggggcccgtcgaagcgcatctggcaactagacaccggggtgccagggggcgattgcttaaggagaagagggagaggcgggagactggcgttttcctctgctcgatcatgtgcgtagtggccacgtagttctggtggaagttgtggagagatcccgggtcgtcccacatggggctgtgctgcacctggaagcccgggtggtgggcgtcgggggcgaggagggcttgtagtaaaccgacccggagtgcggcatcatctcctcagactgggggggcaggtggctgtgttgctggtagttgtgcatctgaatgtcttctaccttaatggaggac

SEQ ID NO: 80 - DNA, 495 bp, Homo sapiens
  source 1..495: mol_type = genomic DNA
atacaattacccacacaacaaagagggcctcgggagagtcgccagccctggtagcatatgaaccaggcaccggcgttggagctggccagggcagggcgggaggaacgcgaggcctgcgcgcctttgttggtgctttcgccttgatggatttcttttctgacgctttccacatttcccgctcccttttgccgcctgggggtgggggtggggggttagcgggcagttgtgttaatttgccctccactcccggcgcccttacggaatcccggtggccgccagtcgccggcgcaaatgccaagtcaccggtaggagacacaacagaggcgatgacctgaatccgcacgtggcctggttcgcctggaagctgccaaagcgaaggcaagagagcccgggaccctgtgtaaaacagcaagcagcacgtgtcccaggcacactgttgcgggcccttagcctagctacgcatccgcaccccaggcttcaaaggcgcgctgggcg

SEQ ID NO: 81 - DNA, 574 bp, Homo sapiens
  source 1..574: mol_type = genomic DNA
gaaagagatccgtgggcttctgaccctggtttaggcaaaacagggggaggcgccgagaccagctcgagcactagcggattttgagagaaactgaccgcaacctccatcgccttccccctctctttcacttggatgggctgactctacccgtcggtgatttacgacgattgcagcgctagtcacagcctggcgcctggtgtcccctcccttcccaagccccctcagcttttccactgccaccggcgtacaagcaagtgccgagcggcctccgcaagtcggactagcctcccggcgtccgaggccaccacgggcagcagatttttggtccccagcgaggctgcgcgcgttcgtcccgcctccgaccgccgagcagagctgctagcagaagcaggcgccggtactttatataatcctgctgctcgcagggtgcaagagcgggaaaagtgcggagtagggaattcttttgctgcgctgcctcctacgcggagcctgctttccactgctgaaaagtgccgggccttgggaagtgtttttctttcattccttaccgaagcgtttactgccgccgtggtcg

SEQ ID NO: 82 - DNA, 1226 bp, Homo sapiens
  source 1..1226: mol_type = genomic DNA
cggctggctaccggtgcgcggggaccacactcacaaaccgcattccggtcttcccgcccgaaaatccgcgctgtgcgagggacccacaagactggcggtctaaaagggacccgcctcacttggaaacgggctgcggtagccacaaccctccggtagcggtagcgtaaccccgttcggtgatccagccgcacagcgcaacgggtacaaagaaccccactggctaaggccgacctaccagggcttgggggaggggagcggaagacgaggtcgaaacgacctgtccagcagaaaactatccccaagcatattccaaccacttctccgtagagctcattccttccgtgcatacgaagggcgccaatccttcctgtatccctcctacagataccgggtgactacgcttttgcgcccaaagcgcagtgctctggctcagctccctacagtagcgacctccaccgcagattctcatctcctcgctaccgtaagagagatgtaaagaatcagacgggttacgcgctcaggcaccctcatgaagagaggacctgggcttaaaactcattcagtatcaggaaaagagcgccgagcacgggcctattatgccaaagcttctgaaaggggcaccacgttttttgctctatggggcagatgacccctccctaatttcggttttcatccatccccaaggtaggctttggagtggcaccggagactgagctcaaatttgcaggccagggactggggagaagggcgccacactaagagacctgcacccccattctcgccctgtactctacccagagtcgtggtcccctccattttaaagcaaaatccaaaagcaagcacggcggaatcttctggaagggggctaagatggaactcaggaggcgggggtcggtatggaaagagcagatggattatttttttcctctcctggcgaatgagggcgcccccagccacccctcctcataaacaccccccaaggcgcgcatgcgcacttaggtggcgggcgggtacttaaggcgcggccaccgcggctgcggcagtgcgcccaacagcggactccgagaccagcggatctcgcaaaccctctttctcgaccacccacctaccattcttggaaccatggcggcagtggcggcggcctcggctgaactgctcatcatcggctggtacatcttccgcgtgctgctgcaggtaagtctgacggggtttcggtgggagagggt

SEQ ID NO: 83 - DNA, 339 bp, Homo sapiens
  source 1..339: mol_type = genomic DNA
cgtctaatcgatggtggggcgaagggagagccacggcggccctgagtgcaagcaccatcctgaaaccaggagcccatgaggccgcgggagccggaggaccaggaccgccctcgcactgccctgtgacagctcccggcccagctctctgccgctctggatccgtcactaagcagctccattaagtgcccggtcaggcaggagcggcagggaaacactggggcacccacatctgaacagagaagatgcccaccttgagaaggtacctggagaagggagccacgagcgacgtttatgctgagaagcggcatccgtgcacctcacctccgcccgtccacgccg

SEQ ID NO: 84 - DNA, 292 bp, Homo sapiens
  source 1..292: mol_type = genomic DNA
cgctcgggtggccgagttacacctggctataagtcatcccgtgcacctgactaaatgcatcccgtgcacctgactaaatgcgtcccgtgcacctgactaaatgcgtcccgtgcacctgactaaatgctcccgtgcacctgactaaatgcgtcccgtgcacctgactaaatgcgtcccgtgcacctgactaaatgcgtcccgtgcacctgactaaatgcgtcccgtgcacctgactaaatgcgtcccgtgcacctgactaaatggtcccgtgcacctgactaaatgcgtcccg

SEQ ID NO: 85 - DNA, 329 bp, Homo sapiens
  source 1..329: mol_type = genomic DNA
cgcggagagacacagggagagggaagggagttgcgctgaaaagacgcaaagatacgcgcgtgcaactccctcccctttcaggtttcagaggtttgcaaaccagggctgagaggaaggggctcgggaactcacgttcctctcgccccccttctgtctggagtctcgcccgccagaggctggttaaccccagtcccggccgccgcagacactgcgctgagcttttgggtcctcgccttgcccagcgccagtgcagctgaagtgagagctggtgggaaatgcaaatggctcctggagaaatagaagatacagaatgattctcattccctcct

SEQ ID NO: 86 - DNA, 416 bp, Homo sapiens
  source 1..416: mol_type = genomic DNA
cgcggctgccgcggctgggcttccgcttctgctgcagcgttggggttgactctggggtggggagggccgtcaggtccaggtggtgctggtctgctttaatgagcatcttggccgtgttgccgggagtgcgagcttgccgttgtgcatgagtggcgtgaggatggcctccggctttgggtctttggattgagtgtccccaaagaggccgctgagcttggtgacgctgctcatggagccccggcgcgagtgggtgagctccttctcttgcgctgcaccacagccacgtctttgcgccgatgatcacagacgcagtagacggtgatgcccgagaagacggcccccatgacgaaagccaggatgactgcaatggccaagagggtgacgggaaccagctggtcgggcctttgaggtaactt

SEQ ID NO: 87 - DNA, 1226 bp, Homo sapiens
  source 1..1226: mol_type = genomic DNA
cggccaaggctggcggcggcaggtacccgggtgccccgagggccacccgccgccccgctctggcgcatccatcagtggccagctttcaccgtgtctgccaggcgggactcgccaggtgtcaggctcatgccataagcctgacacttgcgcggggtcgccgtggctcctcagggaagctgggccaaggaggctgggtcgcgccggcgggcgccgaggccaccttctcggggcaggccatccgcgctgaggcgcagacacggcggtcctccaggcccattaataacccggagggcgctgcgatccgacagcgcagtcgccacaggctctatttcccgtggccgcctgcctccttccagcccagttcgagtggagggatactgggcgcccactaccgcccggccggagggtgggggtccctctcgcgcaggctgctttggagagtgtcggagagagacagctttatctacagcttcacccattgtatagcacaacagaatcgcatttagcctggagaacagccgtcggggcaggagttgtgccttctctaacaattaggctgatcgttaaatttgctattcctttcttccggcgtctagggagagcgtgcacaccaaaccctccagaacagcaccccggtcccccaccccgccccacctgtccccgccctgcttagtgcctggagggagaactgactggtcgcttgagggaccccatttcgggcgcgcccttggagtcctggggctgcggctcctgctctaccgggctgcaggcttgctctgccccgtcccgtggcgtttgggaggcagtgaggaagcctccgagtctctgggtcgaaggaagtgcgaatggcacccgcgatagactggccgtctcccccaccccgccccgtctggtgattttaaatcgtctcctgtgtaattgtaaaaagaggaaagctgaataccagacgaccagtatagaatctcttcgagacagcagattgggcaagagacgggctttttttcgcaatacaaggataaacacatcctgaaactccagatagaaacctcaggaatgggcgagagggtcagaaggaacgaagacagtagggaagagagagagagagacagagacagagggagagatgcagagagactgaaacgggaggaagaagcctggaactgtcggttccgtggagagcggacatgctatctgcgcccagatccgaaggcttttggggagcctag

SEQ ID NO: 88 - DNA, 1226 bp, Homo sapiens
  source 1..1226: mol_type = genomic DNA
cacagaagaaaaggcaaccccaggcataaagaggctctcagcagcccctgcccgaacgctcctggcaggcaagggccccatagcaccctggggcctcgagggtcaccgcagacccggcctgggaagaacccgtagagtcccgcagccgcggagggagggggttctcgctaaggcgtcccgcggacaacgccgagaggcacagcttagcgggtgcgcaccggacaggctcgcaacgcaggacggtgccctcatgggagccaggcgagcaggtcaagaatggtggtggggaagcggggtaggcagacctcggcgggggctgcctcggcgctctgctcctacccaaggggcccggttccctttctacccacagttccctttctatccgcctccctcctcagccaatgaagcagtcggggctcagcagtgcttagcacgctcggactatggttttaatagacgtacatggacaagtcgatatagacagatttattacagtcagtccaacatacacagggacgctgtaaacaggggcgcggccggagagcgggtgtgcaaagtgggcgcagggccctggggccgcgccccttgctctgccggctcgactcttgcacggcgggcggtgaggagggggctgttcgcccagacagagggccacctcctagcccgggagcgagcagagggcctgggcctgcagctaagctcaaggctggggtgttctgagatggacctcccccacctcccgccaggcccgcactgcccgctgtcgctccgtggccttaatatagggctccggggcgcggggccagcaggctctaccaggcgcgccggggccgtgtgcggctccactgagtgcccgatcctgggctgggggcaccgtccccccaaacccaacgcccgccccaacacggatccgactcgagctaaggcgctctcggccgggcctgctcggagagggcaacttggtttgtacggggtcgggaaatcctaggcaagtccaggccccgcacatccagtccgcaggcccggccactcagtccggatccagcgcggcggggacgcgggatacgaggtcgtcctccccggccgctgggcctcggcgctcgcctaccgcgccgcgtgccctccgcggagtgggcctccggggcccgtgggaacacacacaccacccgcgccatcccgagagaaattgcaactcatccattttttcgcggcactttctccgacgt

SEQ ID NO: 89 - DNA, 237 bp, Homo sapiens
  source 1..237: mol_type = genomic DNA
cgcgcgggaccggaggcgcacaccggaggggaggtgcccccaccaaaatttaagtgcggcgcaacatgcaccacaggtaggggcactaccagggcaaaaagacttggtgaatgcgagggccgggcctatcaaaggtgcgtatggtggtaccagagcaaggcggaacagctgctcagtggtccgcaaccgggtcaagggacaagaagacgattctggattgtaggccgcccgagagcg

SEQ ID NO: 90 - DNA, 1042 bp, Homo sapiens
  source 1..1042: mol_type = genomic DNA
cggtgactcggaacagaggcagaaaggatgcgaggacttcttgctgcgtaaacagcgcggcgggcagattggcacctggcacctaccaggactggcgcttcgtcccgcgtttccattgtgcccgcaccactttgaaagtgggcgccggaaactccgtttttcttatggggccggggtcgggcaacgtggttcagaggtttcacaagctccagcacagaagtgatccaaaccgaggggtcggctcgaagtcggaggctgatttttcttgctcagtcgctccgccggagctgagccgctggaacgacccaaacctacctgaaagcgcgtctctgcagtcccgctggacttctccggctcgaactcctaatctcagcccggaatccagccatattcatttaagattgcgttttccaaaggccaaagcctttcccggtttcgtcatcagccgccaggttgggtccctaattagcctgtccaccgagcttccttcccggcgccgcttggaccccgcagccccgggcgatctcagcccgggggggaatccggagacgccggctccgggttaagactctgctcgtccccaggaggacggggtcgcgatccagagtgcgatccggagaaaagggctgcatcttgcgtggctggcgggttagactggaatccgtctggggactgcgcaccatccccatcgctccagacgccgaaacaaaacctgaggctcggagaggggaaaaagagttgttcaggctcgaacatacgcatcccagcctcataggtggatggggttaccgagctgagaaccaagccgaggtccgcgaagccgggccgcccccttgctctgggcctcccttcaagcctgcagtctggggatgggttccgagtcaccgcggtccgggcctcggtcaggcttggtcgggcacacagggcgtttcagaggccagatgacaacctgtggcggcaaaaggactctccctcaccagggacccgcgaaggcgcgcattgcaagatgttagcgtgcgggctttagatatccttgcgtccg

SEQ ID NO: 91 - DNA, 1226 bp, Homo sapiens
  source 1..1226: mol_type = genomic DNA
cggggtcccacgcgggcggcgcggtcttccccaggcctccttctctggagacccggttcccaggggacgctgtgcggggacaggcggagccggcccttggagcccggcgcgggcagaggcgccgcgggacaacgcctccgacggcagggggcgctcgtagcacgcgcagtgcgcggtgcgggcgcccaggacggctgcctggccccgggttcccgaggcgcgggacctgccggggtgaggtgcccccagcctgagagccccagctgggtcggccaaggggcctccgggcttccacatcgtccctttttctgctgacgctcgcggggaagcggcgggaagcctgtgaggggcggcctgggagcagacggaccaccaggtcaccggcgcgcgctgccaggcccgacccactcgcgaggaccagcggttcccggaggcgtcggccctcaggtcctcgggggaggccggctgcaccgagccgggtgtcggccttgtccctgtcctgagaggtgcaaacaccggccccggcccaggccccggggctcctgcccagaaggctcagagctgggggccgaccgcgccttacccgcaggaggcccggcggtgcgtccgcgttggtgccactccccggccggggtgagggacgtcggaaggaaggtcccagggcgcagggtccgcgaagggagcactcggaaggaaggctggctgcggccccggcgcctgggtccgctgcggcgccgggccggagacctgggaacagccagccacctgcaaggttgcagcccacctcaccccgccggcggctccgggctgtctgtgggtctgtctcacggggcaaagcctttcttcccacacccacagccaaggcgcgtccgtgcaggggcacacgccttctgctccagccccaggaaggcgctttcgccctgcagtcctccgacggccggctccgccgcaccgcgcaccctggctccggcagactctggggcctggggactcgcccaccctgcgcggcgcgccccccacatgagccgaggttgggaggctgcggggcctctgtcctcccaggccgtggagtgcggcggcgctctgagtccgctggggacctggatggagagaaactggggctcgttcgcgcacacatgcaccatttgttctttccgatcatctatccctttacgaattttatttttattccggattgttaatgcaagacgacagttgaatctcca

SEQ ID NO: 92 - DNA, 583 bp, Homo sapiens
  source 1..583: mol_type = genomic DNA
cggcctgcggtggggaagcgggtacgggaggcccagctctccccaccagggtcactggagaggagcagacgcagccgtccgccaagacccgggcacgagtccccagcagctgtcactgccccaaactaacgtgaccccacgtccaccagcaggtgaacagataaaccaacggccgatccacacaatggcaggccacccagtgcccacaacaccacgactctccagactgtgcgagcagagcacccgcaagctccctcggtataaactcaacgcagacaaacccacagggccacagggcggcgcggactgggaaggggcaagaggggtagccggcgatggccatgtacactgtggcctccgccctccctccctgcccggccagggcacctgggacggcggggggagcggcagagttgcgccctcaccttgcagacgctgctgctggcagccccttcgctgtgcagtaggacctcaggcacgggcacccgcagctccggccgctgtaggtagagctgcaggagaaggtctcggcagccctgcccagcacgctggaagaggtggagcagggctcaccaggcggacg

SEQ ID NO: 93 - DNA, 560 bp, Homo sapiens
  source 1..560: mol_type = genomic DNA
aacacagttatgacacagtgagcccacagcacagttagcctgcagcacagtgagcccgagacacaggtatgacacagcgagcacatggcatggtgaccctgagacagggttacgacttggtgagccccaacatggtgagcccaagacggttacgacatggtgagctcacggcacagtgagcctgagatggttacgacacgatgagcccgcagcacggtcagcctgagacggttacaacatggtgagcctgtggcacggtgagccaagacagggttacgacatagtgagcccacagcatggtcagcccgagacagttatggcacagtgagcccgcggcacggtgagcccgcagcatggtgagcccgagacggttatgacacagtgagctcacggcacggtagcccgagacagagttacgacttggtgagcccgcggcatggtgagcccaagacagagttacgacttggtgagcccgcggcatggtgagcccgagatggttacgacatggtgagctcgcagcacggtgagcctgccatggcctcaagggttaagatgtaaac

SEQ ID NO: 94 - DNA, 336 bp, Homo sapiens
  source 1..336: mol_type = genomic DNA
cttctcagcaaaggccaacacgtctttctcgagtccaagaccatctcccgttcaagggccctcaccgcgctttgtgagtctgtctcgtgggcctccttcagtggtctgtgaccagcgggaatgaatgggacgtgttttgttcagccctgactcttggacgctggtggcagccgcgggcgcccactcacctggcacgtggacgaaggtgatgagcgcggctgacggctcccgggggcagtgtggggtccagtctgaagccgacgccctctcggtcaggctttcagcagcagaaggcagtggactcttcaccatttcttccaacactttctcacgtag

SEQ ID NO: 95 - DNA, 646 bp, Homo sapiens
  source 1..646: mol_type = genomic DNA
aaactgaggcaaccaacttattcgcaagagtaaaaccctatggggagaagcgattcctgcccctcttcccccggcgaggaacacggctggagccacccaggcgccttccaggctaaggcgcctttagggcgcgcaggggtgagggagagggtggggagagtcctaattattatgaatttctaaaggcgcagtaattattcacggggagcaggacaaaccatggctaggcagggaaatcgatatatttgctatcgaaagttcctgctcgcctttaatgcagacgaatgggggatgcagcctcattattttccgtggttaggctcgccagcgtggggcctgatgcagcgtgaaatctatcatcattagacccgggatggagcggcgggggggagtttctcttacttaccaaaccgcaacaacaaacaaacaacgacgaacaaccgccccctacaaacactcattctcacacaacgttgccctacctccctcgccgcttgccctggccgctgttgcacactcccctgggggctgtctgacgccctagagcagacactgcggtcacttaaagtgcgcccagttcctccaccgcagcggtcacaccgttgatttgatccagaaataagacggatagtaccgagcgttggcg

SEQ ID NO: 96 - DNA, 330 bp, Homo sapiens
  source 1..330: mol_type = genomic DNA
cgccgccgccgtctgtctagactcaagcgactgaaggggccaacagagctggtgtttaaagtagaacctgcccagtccaacagcccgagcagggagcgatttcggggatcgcgggaaggaacgcactcgccaagggagggccgggtgccctcgccaccggctcattcctgctccggttttgcccgatgcgcgtccaggaggttctggcaggacgcactgcccctctgccccggccaaggaggatgcggatactgcccgcaaggttcggcctttatggacccaagtcagccaactgggccgagtcctgcggacaccgaaacctccctttcg

SEQ ID NO: 97 - DNA, 1226 bp, Homo sapiens
  source 1..1226: mol_type = genomic DNA
cgcctgtgctctggactcgctgtgcagggtccggctccgaggcgctggtcggcagtccgaacggagggagcgagacccccaagagcaacggcggcagtggtgggggcggctcgcaaggcaccctggctgcagcgccagtgaccagatgcgtcgttaccgcaccgccttcacccgagagcagattgcgcggctggagaaggaattctaccgggagaactacgtatccaggccgcggagatgtgagctggcggccgccctaaaccgccggaaaccaccatcaaggtatgcggggtccaggctggggaggcgggtgtgcacctatttagcgggaagtaaatgccaactgccaactccctgaaacgcaggccaggaatctgggcctggggtctccctgcccgggcgtgcagattgaccctcgtgacagctcctaggcaggcattgctgccatgtggctgactctgtccctttcctggttaaatagacaaggggtgggcgtgggggaaggggatagagtgcctgtgcgggtgacaaggaatttctggggacacgctctctgcggccgcagaccaattgagtccatgtcctttcactgctcctcccatacacacactggcctctggcaccccgggggcctggccacctgggcagaaggaaggagggagcgggctggatcactccccaaacctctctcggagggattccagctccaggtggtggtggtggggtcggtctctacccggctggtgtctgctttggctgttcctgccctgcgaacactgtccccggagcgggaccagactactggccctgagcatcgggccaagtccagctactgaacctgctccgctcctctccccaggtgtggttccagaaccggcgcatgaaggacaagcggcagcgcctggccatgacgtggccgcacccggcggaccccgccttctaccttacatgatgagccatgcggcggccgcgggcggcctgccctaccccttcccatcgcacctgcccctgccctactactcgccggtgggcctgggcgccgcatccgccgcctccgccgccgcctcgcccttcagcggtcgctgcgcccgctcgacacgttccgcgtgctgtcgcagccctacccgcggcccgaactgctgtgcgccttccgccacccgccgctctaccccgggcccgcgcacggactgggcgcctctgccggcggcccctgctctgcctcgcct

SEQ ID NO: 98 - DNA, 485 bp, Homo sapiens
  source 1..485: mol_type = genomic DNA
cggacttgtccccgcagcccttgcggacctcctggtcgtcatggcgactgtgaaatgtggggtggggagcatgcgttcgaagccatttgcgcgggcagtccctgcgtgtccccccacgtgctccccacactcgcaggacccccgcctccgagcttccctgagcgtgcagcttccagtgagggcagccccacgcacagccccccacaccctccccaacgccttcagcccccggtgcgcgtagtccccaagcccacacgtgcacttccaccttgcggcagctgcacgtacagccccccacacgctcccagccccactggcgcagaacccatcaccgctcgcccttcacgcgcttcactgggagtgcagcccccaccccgagcgcgcagctccacgcagccttccacactctccccagcgcctgcagcacccccagtgcgcacagctctgccagtcgcttccccgcgtgccgcccctgcaccacttcg

SEQ ID NO: 99 - DNA, 564 bp, Homo sapiens
  source 1..564: mol_type = genomic DNA
cgagcacggcctcggaggctgagaggcctgggaggcagggccgcgtcctgcgggctccacgagagccctgtgaaatgccagtcagatcctggcccagtgccccaggtcctcccgcagcctcggcaaaccctcccccggcgctggaaaccttcccctgccaggcgcagaaggccgcgcgcaagcacgcagctcacagtcaccgccacctgctctgaaaccccaggttcagcgcaggatcagagtcccccatccccaccccacgcaagggacgcaggattgacagcgaccctgaagaaatgcagtcgagagctattgtgaaagggcgctggcccggaggccccccagcccgcgcaggacaggccagttaatcgccttggtcccaggcccctattagcgtgtcatctgcgacagcaggacacgccttcccatgccagccgctttggggcccgtgcggccctttttccctcgcagctgctgcaacaaatagcagttcttttgagggaaggaacagtgtggtcctcaaagttcataatgaaaatcttggagaaaagtcatagttgctaa

SEQ ID NO: 100 - DNA, 1226 bp, Homo sapiens
  source 1..1226: mol_type = genomic DNA
cgagcgggacgagccctggggctcccacaccgctggagactgcggcgagttacacgccattgggtcgctggagctgggactgtgctccgggggcatctccccgtttttcatgatcttcttgatcttgatcttttgttctgaaaccagattttcacctgagttggggaacaaaggcacacgttaccgggacactcagaggtcgcccgcgccttccccggggcggcgactggaggcatcttcggacctctgggcggcccagccctcctggcgtctccccgccgcttgcggcctaccgccaagaagctatgccttaggcaaaccatggagctctggccccagagggcgccctggccggctcggggcgccccggcccagcgaggccacgaccccaaggcttgtttttgtttgggctgggggtgggggacgccagcgcggcgggtgtgcgcccgagtgtcctcggcggcggcgcgcctcgtgtaagcctgcgcaggacgcgcggaacgcggcgaaggtctccaaaacaatcgcgcgagccggcggggaaggggcgtggggcgggctacgcgcgcctgtggtcagaaaaaggagcgagctcccaacctcccgtcctactcccttcactccctctgcagctcagagaggccagaatctgactgcagctcttggcggcgctcgaacattctttatgcttccgtaggccccagctgggttggaggagtaaaaggaatgggacaatccaggaagacttctcacgtacaatagcgccccccacctcgcgacccttcggtggccgtggcctgaagctccgtaggctcagggcacgacccttcagtttctccgctccaggcgacgttctcagccttttcttagcctgagaccaccgcgagaacaccaggccttgctcacccccgaggaggctctacattgttaagaaaaccagatactgctctgggctgcctaggccgccctagaaataaccctccgcttgctttcaacccgcgaaattggccccacagctccggaggcccgctacggggtgggggcggggggcgcggttagtgggaggtatctccagaccgtgatgaataccatccccaccgtctcaaggcctgactcaccgagttaaagcataggggctgatgtctttgggtctgttagtttctcacgggtactgtcaaaacagctccagtcccatcgagactgaaccgcgcgaccaaccagacgtg

SEQ ID NO: 101 - DNA, 331 bp, Homo sapiens
  source 1..331: mol_type = genomic DNA
cgattcaaagatgccggcgtcccgcagtgtgcggttcgtgcccttaaccacccgcttctttgtttcccgcccctctgctttcgcaggagctcttgtgcttgagttcagtgttagtggtagcgtggctactccacttggaggtggcggccgtctgaccgtgtgttactgctttgccgacggggcctcccggccctgatgcgtgtacactctgcgggctgcaccgggtggctctggttgggggcgaagctgtgttgactgggagacgttggaaattgagacatggagagatgacgggagtgcgtttctctgggtttgatctccatcctgtttt

SEQ ID NO: 102 - DNA, 1226 bp, Homo sapiens
  source 1..1226: mol_type = genomic DNA
cgcggtggcccccgcacccggagcgtcggcagcacctggagcggttagggctactctgccgccgccgcccccgcggtccccgccgccgctgtccccgccgcggtccgtgcgcgcgggcgccagtgcaccaggagcgcgtgcgccaggcggaagggcgcgaaggcccggtgcgcccagctgtgctcctcgatgaccgcgtagcacgaggccagcacccggttgatgagaatggtgccctgggccgtgagcggcgcgtaggcgccgcggcctcctcgcttagggtcacgctgtgcacagcggcgggcaggagccggcggtccccgtcacgctcggccaccacgtacacgcgctggcccgggcgcacgcggctggcgaacagcgcccgaggccccagtgcgccccggaaggcggccccgagcccgaggacgcctcgggctccccggtggccgagtcgttgtgcggcgccacaaagagcaggtgcgcggcggtgagcagcaggcgctcgcgcggctcccgcgtctcgatcacgtagaagccttcttggcgccgtcgtcgcggtccaggaaagtgaggaagtcgctgtagagcagccggccctggtcgtccgccgccagcacgcggtccccggggctcaggtccttcaccagcttggtgccgccctgctccaggtgaccgtggccgagcccgggaagcagcctcccgatttggccgccaccgagttctctgcgggtgaggagaagggaaagaagagaggacagggcattgagttccgagagggaggcgcgtctcggggaggagggcgcacgctggtgcccgcgctcctaggccaggggtgcgcaaggcgcggggcggggcgattgattccaggcgggtcccgcacacacctccctcccccaaccccactgccccagggacaggattccgacacattccttaacgactcctaagtctgggctggcggtcacagaggttggctggcgtttcctgcagaggccggtgacactctttccttccgccttcctccaaccccacccccagctccgcacacacccggcctcgccgcgaatccatccagaggggtttgctctgcacttggattcctcaggaagcctccttgagctcccgcgggcgccgggcgcaccctccttccctcccgccccggcgcggggcgcggcgcctgctcgcgcactcagcacccgggctgcgcggcggaaaccctggcccgc

SEQ ID NO: 103 - DNA, 337 bp, Homo sapiens
  source 1..337: mol_type = genomic DNA
cgcacggcctcctcgtccctctcgggcaaggctatcagccagagcacctcccctttgacactgaccggcgtccccagccccgccttgttactgcatccgtctgaggtacaaggttagaagccacagaacccgagctgcagcctgacccacacaagctgcgggcgcaatggcgctttgcagacaagacagcctgcccgtctgcccttcttctcggcttcccttaagacacaaggtgacgacacggacggtttcaagtgagtgcatctggaaataatctcagtgcaggggaggggttgaggccaaagatgtcgtgacctggtccccgctgtggccaggc

SEQ ID NO: 104 - DNA, 1226 bp, Homo sapiens
  source 1..1226: mol_type = genomic DNA
cgaggtataaagaccaacgcacgacgggcgccggggcgccgtcacctgcctgggttaacagttcctgccgcgggggcagggagaaaggcgctagaggggcgcggggtgccggcggggccgagcccgggcgcgggcgagccctgggtgccaggccctgcgccgcacgccactctcgcagcgctgccgccgggaaagtgccggcgctggctctccctcccctcccgggaatagcgggcgcaggcacgcacatccacacacatccaacacacacacacaccgcacactcacacacccgggtggccgcgcggggctccgagccgcgcaggcctccttggcagacatcttcctgtgggaggatttcaaacgagccgtgaaagccagaaactacacgggttacaactccggaatccctggccggcctcggcctccctccccacctccggcccggccccctccgttcggcccgtggccaggagccccaccaaaggacaaagctcacacgccgaaagcgcggggagatccgggggcgcagagatgccccgacggcccttctaccttctgaccctgggacccacggccaagtgccgcgcagccgaggtcggccttgctgatctgtggtggcccagggcgacccgaagcgcaccggcttcagggtgcgccctggccagggctggggtggagggtcacataagccacctggcatccgagggtggccgcctcgccccctcaacgccgctctccagggaaggaggcggcggggtcaccagggcgcagctgacagccccaggccccggccagggcgcacccaaagaagagggaggctgctgcctgaactggcctgggggcagagggaagtgggccccgcgctgtggaaggccccaggctggagcgccggggacagctgtccaagcggcgagggctaggagtggcctcgcctagcgctagccggccaggccctggaggtgctccgcgccttctcggccaaaagccagctccgcgccgctggcggagtgggagggaggcggccaggcgagcgccccgccttcgtcgcccgcggtcacaggggcaattacactcctgctgcagacggttaccagctcgcccgcgcatccctctctcggctcccctctcctcccctctcctctcctcccctcccctcctccgcggaactgctggctccatattagcgagggtgccctggcagcgcgaccgaggcaactacccct

SEQ ID NO: 105 - DNA, 468 bp, Homo sapiens
  source 1..468: mol_type = genomic DNA
taggcaggaggaggaggaggaagagggcacctgctgagatacttcactggtctgagggccccttctgccccagctagcgcttgggtgagggttgggcttgcggagccgtcctgtaaagccggggatgcaggacgcattgtcaccccctcctgccgctcttacgaaacactcttaattgagtccgattcttggtgaatcagccttccaagaaccgcgaccgcagcatcctgtgccgcttctgtgttccgcatttttctctttctgcagcgtttcctctcattctggatggaaagcctgtttgtctccctcaatctttggcgagggtggcaggcagccaggcggccattacgggccgcgcctcccaccagccagtcgctggcaggagcgtccggggagggagcagaccccgttcaccctcaccccagaactgaagcagcgaggggacccagacgagctggaatgcaggcg

SEQ ID NO: 106 - DNA, 1324 bp, Homo sapiens
  source 1..1324: mol_type = genomic DNA
cgcgctccgccagcaagacctggcgtctgcaaaacacaggcgtgaggggcgtgcttactaatgcctggtgttccagaaggcggctcccgccaggcctcccgagtaggaaggagagggctcggcccgccagacacacacacacccacctgcacacgctgcttctctgggctctgcctcacggttctcacgctgcagctccggtctcgcgcctccttttacgttgtttaggtttctttctgacatgactgagggaagtgcggttcacaggcagagctgcctggggagagaggtggcaggcggcccgttgttcaccgggccccggcctcgctaaattcacacctcggcggcgtctggcagcttccgcggcgtgggctttagcacaacacaaccccgtcggccctcagagctgcagggttgccgacgtgccctcctgcgcgtgccagtccctcgccccagccgtcccccgccactgttgtccttgattctggttggaggtgtgatttcccacatacatattcatgtggtgtgtttgcagcgtggggtgtgagtcacacggatattttaatggggagagtgggggggcttgcggaggcgccgggcggatggacgtgtccatctggctcgtggggagacccggggccctcagatggtgcgatgtctgaggctgccctcctcacattgatgggagccagggcctacttggcttgctgcccccaaggggaacgtggccctagataccgggggaggccccgccgcagcctcaagtgactgactcaggcccccaggttagcacagctgccacaggcctggcggaaactccggatgggggtctgaaaagcctttgtggggtcggggcaccacacgggttgggctgatggcgcgctgggggcggggctccagggcacattgtcagatagatgaccccacggggtgggctgaggactgctggtcaggtgaggggtgtgctgggaccggctgggcagtgaccccgagccgcctccggcccccaggagctggagttcctacgcgtggccaagaaggagaagctgcgggaggccacggaggccaagcgtaacctgcggaaggagatcgagcgtctccgcgccgagaacgagaaaagatgaaagaggccaacgagtcacggctgcgcctgaagcgggagctggagcaggcgcggcaggcccgggtgtgcgacaagggctgcgaggcgggccgcctgcgcgccaagtactcggcccaggtatgcgggtggggagactgaggcacgcagcacggtgggcgtgagccgccgtgggccccggtggccagtcctgcccagccggaa

SEQ ID NO: 107 - DNA, 1324 bp, Homo sapiens
  source 1..1324: mol_type = genomic DNA
cgagtgcacggctttgtgctgctccaggctcaccgcgtgcccgaaggtcttgccgcacatctcgcaggcaaagggtctggtaccgctgtgggacctgcgcacgtgcacctcgagcccgtgcggcgtgagaacacctaaggcgggtggggccagagagaaggccgctgagaggggccgcggggcgcaggcgaggcgcgggtaggggaagcgggcgcacggcaggcgaggtggtgagctcgggagcctcaccttgctgcacttgatgcacttgtaggagccgccgcccagcagcggcgggtgcacagcagctccgactccaccttgacgccagcgcccttgtctgcgtgcagcccgtggccacgctcggggtacagcaagcccgccgctgccgtgggcctctcatacagcccggctgccgcagacccgaagtcgccgtagagccctaggccagggccagggtggcaccggcccctgcgctgcagctccctggcgccccggcccccgcgccgccggcagcccgcttcgggccgtacagcgcggccgggtggccaggctccggggcgggttcgcagaagaggcccaggccagcgccacgctccagggccccacacggtcggtagcttgcaccaggtgccgcaggtcagaacccgccaggccgctccatgagtacggtttgaaaggcagggggaagggctgggcttcgtccagcgatgggcacattgacttctccgaggctgtggaggcacaaggggagcgtccggtcaggcttcagacggcagagcggaggcgccgggctgcggctgcgagcgtggcgtgttcgcggactgcggggcacccagcgcttgtagaacactgcgtgcgccaggtgccaggctcgccccagggtaaaacgtggcccgggccctggggcctcgcaggccactataggaggcagaacagtaaacagatcattaaaagacctcgggagaggaggtcgtaaattctgtgcgagtgcagcggaggcgctcggcgttccgaggatcttttctggctggacccgcgcacctggagatcctacagggaagggcgcatctataaatgcccgtagttgagctgttaagaaggagaaggtggcggcgttcggcccctcacagcactcaagggcggaagggtccagccaccagcgcaggcactctcccagcccctacatgttcccattcaattctttttcttcccagaatccaagaggaaggagggagaaggtccataaatgcggccccgaactacccgaaaggctgctgtcgaataaacccgaaggtattaagatttcacgaagttaagtgcc

SEQ ID NO: 108 - DNA, 323 bp, Homo sapiens
  source 1..323: mol_type = genomic DNA
cgagaggcccggccaccggctcgggagctgggccggtcgctttgggagcggatgaggaaggttaggagaagcagcgagatagatcccaattttacaattctattttctttcggtagggtctcggcgtctgggccacgttgagagcgaacgtgggccgagcggaggacacagagtaaaaagcgacgcccgctgtatacataaatccgcacccgctgcccgcccgggtactgcctgctctggcttccgctctcttccgaggctgggcaagtccaaaagttcccgaaaggggggtcaaagaggggcgctcagtgcacgtgatttcg

SEQ ID NO: 109 - DNA, 770 bp, Homo sapiens
  source 1..770: mol_type = genomic DNA
ttcttccctctttactccattccctaactcctgcctcgtaggcagcggtggggagccgcaggctgctccccgagtcccaacgctactagggtgctgtaacagtgatgaggcggcgttgcagtctggcaggcgctgcagcgggcaccagctacgttttgctgtcggcggcagttacccggacgcaagttaaggggtaggcgcgcagaggcggttggaggcgggcggccggcccctgacctcggagagctgcgccgagcagggcggaaaatttatggtctgtgtacactttctgctccagagccccgcgcaccagaaggatggggccctgcgagcgcgccccaggccgcgagctggggccgcaggcgccacggccatgtatagtgcccacccgactctgttctgcagcgctgtggtcataaaccttcccttagagggcgcggctgtaaacgaaagtccggcgagaggtggatgcaggccttgccgcccggaccctcaggtataattctggaaacttgcacggaagcctgggtcccgccatcttccctgttgtggtggggtaagaggatagtttatactggctgtttggaatcccgcgcggtggatgcaaggtgggattccagccagcgacgcgccgcgtggccgggagtgcagacaggtcaccggtccccgggtgtctggaccgcgccgcttcctactgggcttacttcgggatctagacttcggcctccgtccttgcaagggcccagatttgattccggttcgtcgaggcg

SEQ ID NO: 110 - DNA, 339 bp, Homo sapiens
  source 1..339: mol_type = genomic DNA
cgaaaccctcgaggagcgaccacatgacccccgcgctgtgagctgctggcggccagcgccctcgcctcgattgtctgcagccctgtagtgggccgcgaggcacgcaagccgcaactacagctcgccacggggcacacagacaggcatgcaccaccactacccgtcgcccggatacactgaactggttttgaagcctgaactgcagagatcctaatatagcaacgcaggacgtttacggccacccgccagtgacagccggtgatgtgatgacaggagggcctgggtgaaaatgcaagacactcactctcagagaacgacgactccacttgaaattcatgt

SEQ ID NO: 111 - DNA, 218 bp, Homo sapiens
  source 1..218: mol_type = genomic DNA
cggccgggtggggaccggcgtggcgtcctgcggaccgcgtggagtttggggtgtctgacatcgttctccacgtggctattttcagcgttctcccggggaagtacttggaagagatcgccacacagatcgcacgcacagcatcaacgcgctgctgatcatcggtggattcgaggtacgttaccgtttctctcttgccggtcttgcaggtgtgagccgcg

SEQ ID NO: 112 - DNA, 1324 bp, Homo sapiens
  source 1..1324: mol_type = genomic DNA
cggcaaccgagccagatccaagaggggtcggcctcgtctaaattggtttcccaccaatggcctcggatcagccgcggctgtgctgcgggagccctcaggacgcggctggggttggtgcgcggggcccggaaccccaaacccggctcggttcggcaaggttcagggagacaaggtagagaaggctggggtgagcgagaagtcgggcggccgatcgtcagggccacgagcctcgccttgccttcttggaatcccacccaactttaaaggcccaaagatcctgaaaattccgaaacgaaactgcgagctggtctccagaagtttgagaacggtctcccaggctttccagcgtcgtcccgggattctcggacaccacaaacgccatcaaccacgagcaccggtgtccgtggctattgccccgaatggtccccatccgcgtccccgggaactccctcggctttcgcgcatccaggtccccagccccagctactggtgcgccccgagcccctaggtgccagagcggtggtcggccgggctcctgcccagtctcggctcctccctcctccccaccagaaggaaaaacttgggcccttcgagaaccctgtggaatgttctttgtaatcaatgtacatccgcttccacggcacggcctcgtgcaaaatcgcgggtttcggggccttggagcaaattgcgcttgtcagcggcgacgtcaggaggacaaggggaggggttcgcggctgaaactgcagcttcgcagcacagagccattttaggctgctccccacctcgcgggcccatgggaaagccggccccgggagggcgcgcccaaacgcaggctggaggagccacggacgcgtcctggccggcgtgtcgccatcgttcagcctcgctgcccaggtgggaggggtcacctgccgcggggtctccaagccagtgccgcttgctcccggccccccccactgacagcacggcgtccgagtgaccctgtctagcctcgttctgcgctcctgcaaaccacgttgctgcgctaactacaaacctggccaacatgtctttgtaaccctatcatttaaaaacgcttccaggcacctggccgctgccagatcaggttcgcgggcccgaggaggtcctcccacctgcccccgccagccccggggaccgtgcgcggcctccgtgtggcccccgcccacgaggtccctcgggcaggaaccgccgcgcgacctctgttcagcggccgcgtcctggccacgggcgacccctgtcgggaaccctgttcccggctaagtgcgttcccgcattccggtggctctcacccgagctcgcgttt

SEQ ID NO: 113 - DNA, 337 bp, Homo sapiens
  source 1..337: mol_type = genomic DNA
tacagttgatacccaccatccccatcatcgctattagaattggggtcaatacccatcatcactgttagacttgaggccgatacccgtcatcactgttagactcggggtcgatatcatcatcactgttgactcggggtcaatacccttcatcactgttagactcggggtcgatacccgtcaccactgttagactcggggtcgatacccgtcatcactgttagactcggggtcgatacccgtcatcactgttagactcggggtcgatacccgtcatcactgttagacttggggtgatacccatcatcgctcttagactcggggttgatactcgtcatcg

SEQ ID NO: 114 - DNA, 592 bp, Homo sapiens
  source 1..592: mol_type = genomic DNA
caacatctgtcatcgctgttagatttcgggtcaatatccatcatcgctgttagactcggggccgacatccgtcatcgctgttagactcggggtcgacacccatcatcgccgttggactcggggctgaacccgtcatcgctgttagatgcggggtcgatatccatcatcgctgttaaatttggggtcgatatccgtcatgactgttagatttggggtcgatattcctcatgactgttagactcggggctgatatctgtcatgactgttagactcggggccgatatctgtcatcctgttagactcgggtcaatacccgtcatcactgttacactcggggccgatacccgtcaccgctgttggactcgggtcgatacccgtcaccgctgttggactcgggttgatacccgtcaccgctgttggactcgggtcgatacccgtcaccgctgttggactcgggcgatacccgtcatcgctgttggactcgggtcgatacccgtcatcgctgttggactccggtcgatatccgtcatcagtgttgggctcaggtcgatacccgtcatcagtgttggactcgggtcgatacccatcatcg

SEQ ID NO: 115 - DNA, 780 bp, Homo sapiens
  source 1..780: mol_type = genomic DNA
cgacagcttcacagtaggattattgtgataaaaatgactcaagcgatgcaaaaagtttcatctgttcccagaatccgagggagaactgaggtgatcgttagagcatagcgacatcacgtgcggtttctaatgtccctggtggcggatacgccgagtcctcggaaggacatctggacaccactttcagccacctccttgcaggggcgacatccgccaaagtcatcctttattccgagtaataactttaattcctttctaacatttacacggcaaacaggaatgcagtaaacgtcacgtccgtcccacggctgggctgccgttccgtttcctccacgaacgggtacgcgcttccatgagaaaggatatttggcaattttatattccacagtcaggtgggtctgcgatagctcatttaatgttaaacgccatcaggggcctctcctcccgtttctgccagggcttttcttgtcttctccttggtcatcatcatcatcgtcttcctcttcctcgtgggcagatcttctctggtgggggctggctgctggctccgagggggcatccgcagtccgtctggtcgtctcctcctgcaggctgggcagctggccaccacttctccgactcgcccctccaacaagcatcgcagggcactgtcctcgggggtacagaccgtggtcccacattcgctaccactctgttccacgtcatccaggtacacgagctgcgtgtaggccgtgctgtctggggctcgaggctctttctgctggtgctcttggacgggcg

SEQ ID NO: 116 - DNA, 1324 bp, Homo sapiens
  source 1..1324: mol_type = genomic DNA
cgtgaacgccctcgcctcgcacctgtggcgcctgccgcggggcccgaccggaggcagtggctgtggacaagggcggccccggcgtaggccaggatttccccgcagccggaccggcccagctccgctctttcctgggcgaacagcgcccacctccggcccaggcggcggcgcctccgcccgcggcgcggacagcgagagagaaaccggctcctccaaacctggtggaggagcgctgcagagaagcgccgggctgaggctctgggggccgcggggcgcgaaacgccgcgcctacaggttatgcccgggagaaggagacaggtcgggctccagccgctgggcgctcctcacctcgaagcgaggaaaacaccgggccactgagatcaggggaggtgcgcccgatacaggtacccggcacgcagacacgcccacccccaaacaccgagaaacaaagactgcccgagccacgcacgcctgcgcggccagcgctgggcgcacacccgcgcccacacagcccgaagggcgccacgcaacccgcaagaacaccagctccccgcgctcgcaggctctccgtgccggcccgcagcgcagagcagtcccggcgccgctgcctaggtgggccgggaaggcgcgccaccgcgcatggcaccgcggctgccgaggaacagagggtgccctgcgcagcccagctcgtcccaacccctccggtgcccactgctgcgacgcctcgcaggccgtgtcccagtgggccctgggcacagcggtcactcaccttcctgcacagcctggatgggttgttggcctcgatgaccttgatggagtaggtgatcttctcgctctgcaggatgtcatcgttgaggctcaggtcggataccgccaactggaacaccctgtcgtccttggccgcgttctcctcgaagatggcacctggaggagagaagggatcaagaccggactggacaagaaccctctccccgcttcccttggcccgagggtcaaggcactcgcagaccgatgattgccgggtggtgggaagtctgaactgggatttcagcttggcggggtagattaaggctctccgatctccacctgcacaggaatatgttcccctccccccaccgccctcagcgcacacgtgcacacagttgacatgagttacacatgcacatcccacaggctgccaccaggtcacacacgtgtctcacaaccaaggcaccacatgtcctagccatgctccacacaagggactaagccaagctggggtggccccaggcagcgcccaaatccttgacatggttccataaatcccatcccaggagccaggaag

SEQ ID NO: 117 - DNA, 609 bp, Homo sapiens
  source 1..609: mol_type = genomic DNA
cggggggcgccgggcgcaggccgggcatggggcctgagcttaggccaggtggaaaggtggaaggacctagtggggacagacgaagagaccgaggagccgcccgcccggctaaatggacacaggaaagcctgagtcaggcttctcataataaataaactgcacgtgaataattcagcagtcgcgcgggggccggttgcatcagcgccgccgccgccgcgcggcgcccaattccccgcggaggggagtagccaattaaggcacttgaaaagggagtcgggtggaagatcccccgccaccagtatcctggatttacccaggtcgagttcagagagcctccccggtcccaaccccctcgggccccatgggtcagggggccgcctgctgggaagtgctacctcagctaagccgctcaggtgtcccgagactccgggcaagggaagacaggcggcgagacggaccagaataagggatgcggacgtctgacctgtggaaaacccagcatctgcagtgcccagccgcacagggaacgtgggcggcgtggttccaagcacagtgaagacgccaggctcggatctgtgctcagagccgggatccctccgggctgcttcg

SEQ ID NO: 118 - DNA, 298 bp, Homo sapiens
  source 1..298: mol_type = genomic DNA
cgtagcgacgacgtggtccttgtaggaccaagcattcttggcattctgtcgagattgagattccagagcatcggatgaacgggacttatagctaaggcgtactcacccacccctcccttcgtcttttacgcacgcagcaactcgtcctcacgtgcataggggtagtggacagtgttgacttgaattgctgtccctcgttccaagcctttcctgtgaatgaacccgaaaacactcgacaggtcgtgaataatcgttttaatgagtgtgcaaagcgtgcgacggggtgcaccgactgtcg

SEQ ID NO: 119 - DNA, 287 bp, Homo sapiens
  source 1..287: mol_type = genomic DNA
cgctggcgggggtagggggtgtgggtgggtacggagtcctttcactgcccgtcggcagttggcaccagagcggaaagtgtgtcccctcccccgcctctctcccttgggcagtttgatttcccgccagctcagtcggagtggcctggaagagcgggctcaaactccaaaggcagtcgtgggagagtcgtagcggatacgtttcccctgacatgcgccgggtgctcgtctctgccccccacccgcccccaaaacccaaacctgcaagtcgatgttgtccggtgaccccg

SEQ ID NO: 120 - DNA, 1324 bp, Homo sapiens
  source 1..1324: mol_type = genomic DNA
gtgcctaaagttacctaacgatacaaaccggatacaacgcagaggaaacagaataccccagccaagcaatttccatgtcaaacatcatccgcgcggctgctccatctgtgaacgtgagtggtcgcctgctccctcttccgcggcggccgcttcctcataccttcacacggcgcacaccggccggcgcgtccagctgcctgcccagtggccgaggctcttccccctcgcccagtcttgagctggaagtgattcctattggccaaggtgtccatgtaaataggtgtgaaagaaacagagctggccgggctctcccttctcgctcagtcccctccctctgcagcccccgctccccctcctcttcctcctcctcccaaggcgattgtcatatgatagctaagaagtggcacattaatgaagcgccgctacaggggtcttttctgctcctgtcaccgcttaaactatcagatggttcgagggaggacatggaggcagccacctagctcagcggagacgcggagcccacagcagcgccctccggagccctaacacgtcgctgccaccatccgcgccgggactccgcagccgagctcggccgcccgcaggacgctccaggagcgtcgcgaccgggcggcacgggacgctgcggggctgagctcaagagcccaggttcgcgccgagtccaaccggacccggacgctgcgcgcggagtgcgcgtcgagtgcgcgccgagagagaagcggcgcgcagcggcgtcctcccggatgcggacgcgcaacttgaagcaacttaaggtgagcagctctctgttccgtccctgccccctattctggccccagtaccgacttacttcccggctatcctcgcgccgttcgccggcttcccctcccgcgcccactaagcccgcaaagttgctggcgaaagagtccgggcgctggctgatcgagcgccgcagccccacccccgacccccgaagtctgttactcggtctggctgaccccgccggtgtctctgtgcatccatgctacctttccctattacccacccccttcccagatccgagcagtccgccggcccgcgcggacccagagcaagaagagggcgaggaagaagatgcctcgcccggccgcaacacgtacagcgaccagaagccgccctactcgtacatctcgctgaccgctatggccatccagagctctcccgagaagatgctgccgctgagcgagatctacaagttcatcatggaccgcttcccctactacagggagaacacgcagcgctggcaaacagtctgcgccacaacctctccttcaacgactgcttcatc

SEQ ID NO: 121 - DNA, 1283 bp, Homo sapiens
  source 1..1283: mol_type = genomic DNA
cgacgggaaaacggggggaaggggggagcccgacacggcacacacacatacacactcgcacacacttccgagcgagtgcacactcgcactcccacccgacagccggccagggacagtcacccccaagtcaatatcgcagtttgaattgttccggcaaatctcccctcgggctcgacggatgtgcgccccagatgtgctgacacatgtccgatgcctcgctgccttggaggtctccccgctcgcgtgtctcttctcttcgcaccagcggcggaaaccgcactagcagcggcggggcggcggcggcggcagcagccacctgaagccaccaacactgggctcttccagcaaaaacgagaccccgattcgtctggcgccccaagaagacatagacgatagccccccgccgggctgcgccgtgcaattccgggctcccgggagcgcgtagctcgctggctaacccaggcagtccgcggcaagttgccctcgaagtccgcccccctcaccggggcatggtcggagggtggctgctgcgctccggggcagttcttctcctgcttccggggggcggggaaggagcagacacagcagacacacagagaaagagaggcagggattattgcccaaaccgccagccgccggcagcctccgccgaccctccctgctccccaggtccgcgcgcagcctgcagcctctcagccgggttccggcagtgcgtccagggcgccgaggggaagggctgggccccggggactggggcgcggtgggggcacacagactctccaggtcggcgcgcccttcctgctcctctgctattttcctgaactccccagaacccccagttgcctggcttccatcgcccgcaactagcgccgctgtcgagattttccgcaatacaactgcaggggtcgttatttcctcgcctacggatccctacgcttcccaactcagagagaggaggacaggtgggggcgggagggggaggaggagaaggaggcttggccaggtgagcgtcctcgccgccgccgggcgtcccagcgtcagctccgcagcccggcacccgggggcggaggcaggaggcccgggcggggaggtgaggctgagcgcggccatgaggaggggaggaggaggctgggtgtcacagcttcaggccaccttggccgccgcctcggccccagtccctggcagccgcctccgcactccgccccgccctggctgcctctgttaccctccaggattttcgagtttctcttcttaaaaagaacttcgtgctgcgggttcggcgg

SEQ ID NO: 122 - DNA, 327 bp, Homo sapiens
  source 1..327: mol_type = genomic DNA
cgtgggtccgcctaccccaagcactttttgtgacgtcacagctccgaggaccatccgggggctttactctcgcggaagcccaaaagttggggtttgacctcatgaccttggccttcccggggggcggaggccctcgggacaggacgcgctgtccccggagtccggagctcaggccagtggcagtcgacccagcccccgagactccctcacgccgctccaaaaccaaaacggagcccaacacgaagctgggtgaagccgtagcttgcaggagccagggagatgcgctctgcccggacttcccgggtcctgttgagacggaaaggatcg

SEQ ID NO: 123 - DNA, 1324 bp, Homo sapiens
  source 1..1324: mol_type = genomic DNA
cgactcggctgacgtttttgacccggccaggaggcaaagaccaaaacgtcagagcagtagccctgttactgaggagcgtcggcagggtcgcgggtagagggggctggagaatgacttgtcagagctcaggtcgatgtggcgcggggcggcctcgagagcgccgggctcctgcgtggccacggccgccgctgccaaccttcgcggggacttagctttgctttccattgactccctttgcaaaagcgcagcagaatcctgaccagccgcaccagccccggcgaacccgagcatgtaatctatttatatggattattacggaggaacagcgggcgttgagtcaccaaaacatttgcttcaaaagactatttctaagcacttttgcaggcaggcaggctcgctccaggcgcgtaaactcggctacgcattaagaagcggctgcttttcgaatactgcaaacccagctaagtccccggtgccgcggagagagcagtgaaaagaaatgtcggaggtgggggtagatcctagtctagacacacacacttgcgcgcacacacacacacacacacacaagattcgcgcggagaaggcactaaaattctggcattccgagagtacgacaaactacacacttggaagtcccgggtcccccgccttccccgcagcaccccccgcccccccaccctaccgtccgccctttggctgcgatcccctcccctctcctcccctcccgcctcgtcacccagcccagtgccacaatcctcctccctccccaaaatcgggtccaatcgctgcctgccaaccctgggactgctgtgctgtgattggcgggtggctctaaggtgaggcggagtatttattaaagagaccctgggctgggagttggagagccgaaagcggagctcgaaactgactggaaacttcagtggcgcggagactcgccagtttcaaccccgaaacttttctttgcaggaggagaagagaaggggtgcaagcgcccccacttttgctctttttcctcccctcctcctcctctccaattcgcctccccccacttggagcgggcagctgtgaactggccaccccgcgccttcctaagtgctcgccgcggtagccggccacgcgccagcttccccgggagccgcttgctccgcatccgggcagccgaggggagaggagcccgcgcctcgagtccccgagccgccgcggcttctcgcctttcccggccaccagccccctgccccgggcccgcgtatgaatctcctggaccccttcatgaagatgacgacgagcaggagaagggcctgtccggcgcccccagccccac

SEQ ID NO: 124 - DNA, 602 bp, Homo sapiens
  source 1..602: mol_type = genomic DNA
cgattggggcctcagccttagttcttccagctggaccctgggatggtgcggggctgtggctctgggaagtatcttggcctgcacgcggtttacatcattgggagcacaggcctcgccctccgaactagaggcccagagcccttcgcgccgagcgtttcccgcctttcccctgtctcaagtcattgcccagaggccactgcgggccgttcctgcctgtccgtggagctgcggcccccgtgttcccagggtgaacgagcacgtgcgaggcggtggtgcttgtatccgggataacaggtagggcggctgcatgcttcatagaggccagcctgtgccgaccgggagctgtaggacggtctgtgtctggggctgcttatgggtattcgcgtgcgtctgccggtgcgcggtccgagcctactcacgagagcgtgtgtgtgtcttctgtctcgtgttgctatgagtttgcatctgtgtggctggaatagcttgtttgtgggggcccgcgcgtgacctgtgtgtgcgttactgtgtgtgtctcaggcaggatagtgacgggccgtgtgactgtgacgccatcatcaggttggtgagatggcgtgaccgcg

SEQ ID NO: 125 - DNA, 599 bp, Homo sapiens
  source 1..599: mol_type = genomic DNA
cagcagacccctttttaagtacgcatgtgataagcaatgaacacgaactgcccagagcagtctccaacactgacacgattcgcttccccaccacgacgccctagcgctactgtgcaacgaagacctccaagcactggttccaatgcggagaccatgggctcccagactctgggaactccaacacgactgcgaaacgaactccgagcgaggactccccgagagctccccgcaacacggacctcacgcgctagcgaacaacagaaaaaaaaaagcgcgctctccctgcccctgaacattcccagaagcccacgcagaccagaccgatgacctgtctccactgctggaggcgagtcagggacccgaagtctctaaacactcgcctctacccgccgccccgcgaaccccacacactgcagacgcgacactcgcaagtttcggggatggcggccggcgagggcatactgcgtctttccggagacacggaatacggcaccagccgtccctttatgatgcaatatgtctgcgcccaggggacgcttgctgggagcagccattttcaaccctactgccgtagagcaggcggagtccctcttttcgcg

SEQ ID NO: 126 - DNA, 1324 bp, Homo sapiens
  source 1..1324: mol_type = genomic DNA
cgaggaggctgcagggacgcgcatggaagagccggtgcgtgggagggtttgcgggggggacatcgcgccccctaggggtgaccccagtgggtcccgtgtgctctccgcggagccggcggagccttgtctctgcacccggcgcgcagcggccccttaaacagtggaaccgtgaggccgctctaagccgaagggctggaatctgggtttctcgggttttattttagaccattcggcaccaagcccgagctcccccgccgcaccgcttccagtcccctttctttccatagagcgaccgaagccggcggtggcgcagggagccgagtctgatgagctcgcgggcggctgaaggccggcttccctgtggggaacgcgccacctgtcggcgccagtgagaactgcgtctgtgtggcgccctcggggtattcggggctgcggggagatgtgtgcctgaagccctcgcttgcggtggggacgtccggcctctttcctggcaattgacccctgaggcgggagagacaacggaattcccacaaagggatccttctcgggatctccccacctcaagacagctaaagctggaggaaaagcccctccggggggtggggggtgcgggtttgccctggattccgaaagcagaaaatacccgagccacacagggacgggcgccgcgttggtagtcggggctacgttcctactccctctacctcccccgcgctgtgtgaccctgggcggaaccccgctgctctctgggcctcagtgttcttattcgtaaactgagggcgttggagagattggtcctctcccaactctgaccttgaaactgatactgaatctgagcagcgtctgtagacacctgtgccttgccttctatttctagccttgaataaatcctggacttttatgtgccatttatatcctaatctcatatatatttaatgtataactgctgccatattgttttctcaattgtctaggttttcatttggatggggttaggatggtccaaattatcccgataagtgcccattaacttaaacctttttaaaaaatgaaaccagtaaaacttcattcactttgcagtgtggacactgctggagagcacccatgtcgtgggtccgcgaggacacaaggaggggcttagagacatgcgggaggcttagatgagaagacagcacccgggcagcggtcagtgttagagagaggacccgtaagaagggccgaggctagagggagagcgaagactgagccaacgacgcacctgagccctggggtgggggtggagcgtggctcctaacccaaatctccctgccaggcagtgtccgac

SEQ ID NO: 127 - DNA, 1324 bp, Homo sapiens
  source 1..1324: mol_type = genomic DNA
accatgagcttccagttcctacgggtgagaaaagaggtgtctgtgtggagggcaggtgcgaccccgggggctagcagagaggagaggctgcgggaggactcgctgcgaagggcgaggggtggcgctggtgggacgggcgcctggggccgtgcagggtggctcctgggtgctgcccgggctgcctgtcgcccagtactggcgcaggaagacgggtccgcgcagcgtctggcaacagtggcctgctccacgcctggaggcgaccaagttctgagttaaaggcgccgggcttagcgggaagtgtatggcgacccagaacacggagcggagaaggcctcaggggacacgaggctaccgccttagtcctcgggcctgcgctccgagcggttagggtgcgtacggatgggctccgggatgttagtggagaggtgacaggagtctaccatcccacggccaagcggtgaggcctccggcgggcgggggtcccccccggggcagggcagggggagagtcaggcatgtctctaacgggctccctagtagaaccgctaggaaagtggccttcaaccctaggcacctttgtggctttcaggtagttgggcgactaaaaattcacttttgcctgcgtgaaatgggacaggtggttgtcaggttagagactgtgcccacgaagtctcgtttggcgggccgggactgacgtggacccgggccctcctcggaagcttgggaacctgtgagcccctcagctcctcgcctcactttgcctgtttgaaaaaaggagttgtttgagagaagagttaaggctcaaaaggttctaaacgaagagcatgcggcctgacgcggagcgcaaagaagcgagcagtgaactgaatctggtgggactgagaccagaggcgtctgtatgcggcggccgcttcgcgccctattaaagcaatctgttatttacgataatcattttgtaattattttggagtggctgtgacttgatgtctgtggtgccccagggcatgcccttactggacatagatacttcagggtgccctttgacgccagtgtcggtccagtgggtgccagtgccgcgaagggagggtggcgggcgtgaatgccccctcctcctgggttgccgcaggccccggcctttccaccttgcttgatcctggcgggtctgccacttactgaggtccctaccggcgctcacgttgtcccgagaggcgggacaaacgtccggcgcgtggcgcggagtctgcgaaagcgctcgggggcctccatcttctgctcgggagtccctccctggggcgggaagaggagtcgcagatctgcccgaatct

SEQ ID NO: 128 - DNA, 530 bp, Homo sapiens
  source 1..530: mol_type = genomic DNA
gagtgcgtaaagggaaatttttgcattggtggatttattgaaagcatggaaacagtcttcgggggtaggtgtagggggtgcaacagcgagacttccccacaacacctccaccgaaggctgggtcgtcgcgtttgagggaccctcccgccaccccgcagcctggcggggcgctttccaaatcgtcgcggcaggctctggccgggcagggcgagatgtgcgcatgcgcggcctgtggcccgaggttccacgtgctgatgaatatgcatgagactcccccgccctcgcgggctaggagagaaccaagcagaggctccggcctgtgaaagtcgctggctaggctcccaaaatagtgcggcggggtagctgcacgtgtttgttttcaaaccgcgctggtttaagtgagttcacactgcggagccgacagcagagggtagagcaacctggggctgctgccattgaacggcgagttgagcgggcctgtcagggcctctccgccgagggccgggggctgcatggtgctcgagccccag

SEQ ID NO: 129 - DNA, 1273 bp, Homo sapiens
  source 1..1273: mol_type = genomic DNA
cgcgttgcccctgcgcctccggaaggggcactcacagctggtcaagcgcgtccttcggaactcggggaggctccgttggggcttacacagagctaagccctgggccagggaggatggatgcaagtacgtgcatgtgcaaacgtgttcgcagatacccgcagcggtgcacactcgggcccctcgtgtgcacgggccctgcccaccaccccgcccgcgttcgccgccgccccactgtcctcatcccgcgggctgaccccaggattggcagaaatcagtggtctaatctagcatgtgggtaaagggatcgatctccaattgccaaaaaaaaaatagaaaatagccggtgttcccagccgctgcgtggcacttggccgcacaaggcgcagggatatcagaggcgctttgcagcgcttggcggtgctgtgtgccggatgccccagagcccggcgttaggatgcgggcttgtaggaagcggctcaaatcaactcctcagtcactgatgaacgcgctcacggctcgaagctaggtcctcgagtgagttaatctacttggtaagtattttgtgaatatgtattttgggaggaaagaagcacgtcgaacgcgtctgcccgtctgtaagcgcgtcatccccacatcctttagcgtgaattcctccgctttttaggagtcacctccaccccctactccgtatgcttcgcgcctttcctcacggccccgtcagaccccctcacctttagattgacacatgccatataatactcacttttaaaatgccctgccagtctctctagggtcccttaattaggtaacccttgccaattagtgggccctgcgctactttttcccttctcgatgatttatttccatgatgcgtattagggaaaataccttaaaggaaaaagaatgttagcatcattggaaatcccgctcctaggagaggatatcgcagctgtgctattatgttttcgcatttgctgatgcaaacaggggaacctctctattccctccccattacctgcggctggggactgggaactttccgcggcattgtccccagccagcgcctcgggacctgcggggctgttctcgctctcacactcacactcaccccggccgccgggcgagagttctgtcccgccccgcccggctgccgcgtgacgtgtctgtttggcggccagtttggccaatcatcggcagctcggtgggtggtgctcctgggcgattggttggcagaatgagggcgctgcgcaaaaacggagaagccgagcgctcg

SEQ ID NO: 130 - DNA, 1324 bp, Homo sapiens
  source 1..1324: mol_type = genomic DNA
cggagccagacctgacggtggaaatctctgagagcgccacgccaggcactcgcttccccttggagagcgcattcgacccagacgtgggcaccaactccttgcgcgactacgagatcacccccaacagtacttctccctggacgtgcagacccagggggatggcaaccgattcgctgagctggtgctggagaagccactggaccgagagcagcaagcggtgcaccgctacgtgctgaccgcggtggacggaggaggtgggggaggagtaggagaaggagggggaggtggcggggagcaggcctgcccccccagcagcagcgcaccggcacggccctactcaccatccgagtgctggactccaatgacaatgtgcccgctttcgaccaacccgtctacactgtgtccctaccagagaactctcccccaggcactctcgtgatccagctcaacgccaccgcccggacgagggccagaacggtgaggtcgtgtactccttcagcagccacatttcgccccgggcgcgggagcttttcggactctcgccgcgcactggcagactggaggtaagcggcgagttggactatgaagagagcccagtgtaccaagtgtacgtgcaagccaagacctgggccccaacgccgtgcctgcgcactgcaaggtgctagtgcgagtactggatgctaatgacaacgcgccagagatcagcttcagcaccgtgaaggaagcggtgagtgagggcgcggcgcccggcactgtggtggcccttttcagcgtgactgaccgcgaccagaggagaatgggcaggtgcagtgcgagctactgggagacgtgcctttccgcctcaagtcttcctttaagaattactacaccatcgttaccgaagcccccctggaccgagaggcgggggactcctacaccctgactgtagtggctcgggaccggggcgagcctggctctccaccagtaagtcgatccaggtacaagtgtcggatgtgaacgacaacgcgccgcgtttcagccagccggtctacgacgtgtatgtgactgaaaacaacgtgcctggcgcctacatctacgcggtgagcgccaccgaccgggatgagggcgccaacgcccacttgcctactctatcctcgagtgccagatccagggcatgagcgtcttcacctacgtttctatcaactctgagaacggctacttgtacgccctgcgctccttcgactatgagcagctgaaggacttcagttttcaggtggaagcccgggacgctggcagcccccagcgctggctggtaacgccactgtcaacatcctcatagtggatc

SEQ ID NO: 131 - DNA, 1324 bp, Homo sapiens
  source 1..1324: mol_type = genomic DNA
cgagagaaggtgagctcggcgggcgcagcgggttgggtgggtgtggcccgcggctccgggaacagacccaaggccccgccaccgccggctagcagcccgacagcccgcccgcctggcgagcgcaggccttcttccagaaactgcgaggatccgagcgtactgtttgtgcaccacaaggtcaagtcgggaagtggagccggaggaggaggggcgaggaggcgaagggtggggagaggggcgccccgcttcctcgccatgtgccagcctttggaagttacccctcggtttttaactttcggggtaatgggaaggaagagcaaggaggaaggggtcgccccggggcaaaatcctgctggggccaggatgtgcaaccacccacccacccgctcatcactgccttctccaccccccggaaatgcggggcagcgctaggcgccaggaggcacccgggagggggagggggcgacgagcccaatggggaccctctctacggaccgcccctcttttcttgaggtcgcctacaatttcaagttcccccgcccggaatgttagttttccatttggagtgggtgcggggaggagggccgcgccccctccccaagactcgcgttcgcccctccaccccctcctgccagcactttctgctcacttctggtgctgcaaaacgccacgcaatttattcgattcgcattttaaaaccagcgattcagaagcagggcctagggcgagggtcttccagcgctggagccccggggggcggggtgctcagtgcaaacctcgttccctcgcgagtgtcgcctccaggctgttatttgcaaagaaacgtctttttgacgcagggagaaatggcaaattttaagtacgtcattaatatggcgccaaaagatttttttaagtataagctcgcttttttttttttttaaggttgcggggggcgccgcccgggtctgggcgcgtagggggcggggtgtgagcagaaagtgtgagtgaaagagtggaggggcggggtatgtgtgtgagtgtgtgaagtgtgagcagacctgatgggacttgcacgggcgcagccgccgctcgggcccggccctggggacagggcgggctaggggcgccccagagtcatggggagtccgggcccagggtgccagcaggcgtggtggtggggctgcgagggagggcacccttcccccacggggcccgcaacgctacctggactccccgccggagccaaacaactgggcggggggttgggggggcggcgacgggggtgtcgggagcggagatcgagtgaataagaaaaaagtggctactccccctccctcgctcc

SEQ ID NO: 132 - DNA, 762 bp, Homo sapiens
  source 1..762: mol_type = genomic DNA
atccctattccttctccttcctctcccttggtaacggcactcctagggctgtggagggtgcggttcactcctgggactgtggagggtgcggatcactcccgggactgtggagggtgcggatcgctccggggctgtggagggtgcggatcgctcccggggctgtggagggtgcggatcgctcccggggctgtggagggtgcggatcgctcccgggactgtggagggtgcggttcactcccgggactgtggagggtgcggatcgctcccgggactgtggagggtgcggatcgctccggggctgtggagggtgcggttctctcccggggctgtggagggtgcggttcactcccggggctgtggagggtgcggttcactcccggggctgtggagggtgcggttcactcccgggactgtggagggtgcggatcgctcctggggctgtggagggtgcggttcatcccgggactgtggagggtgcggatcgctcccgggactgtggagggtgcggatcgctcccggggctgtggagggtgcggttctctcccggggctgtggagggtgcggttcactcccggggctgtggagggtgcggttcactcccgggactgtggagggtgcggatgctcctggggctgtggagggtgcggttcactcccgggactgtggagggtgcggatcgctcctggggctgtggagggtgcggatcgctccaggtgtgattttcatgggaatggaactggctccctaggtggtgttcatttc

SEQ ID NO: 133 - DNA, 270 bp, Homo sapiens
  source 1..270: mol_type = genomic DNA
cgaacgccgggggagggacggtgggaggggtgcggtgagcgaggggctgccctgcatgtaaacagcctcggggtcccagacgagacaagtgggaccgctcaagcctgcagaacccccggtctcggtccaggcacaagagcctcccgtagccccgcctgatctttctcagtctttaggttccaggacatccaaaggagcgtaggtaaccgacgtctacgtagtatttctgtccccggaagactggatctcagggaattattatggaagata

SEQ ID NO: 134 - DNA, 876 bp, Homo sapiens
  source 1..876: mol_type = genomic DNA
cggggcaagggcgcgttccctatcgcaggatcacttgctatggtaagccgcccaccctgcgcgctcctccgcgcggggaagaacctgcgcggcaggacgtggtgttggagttggggcgcccggagcgggagtggggagacctgatgcagagagtctggagtcggagctgggggtgctgcaggtaggagcaggagcggggcggagagggaggcccgaagaagaccccacacaggttggcgcagcggggcttggggaggcttcagcccagaagtggagagggttgacagacgccgcctgattagaaaaagccgggagcttgggaaggagacgggattgaagaagccacccggcagggaggccgaacggcccagagctctccgggtaaaacccgcctgcggtgatctgggaagtgtgtctccacctagccctgcgagcagcggccttcctcccgcccgttgaagggcgctgtgctggagtacgaacccggcccagagaagccactcgcccttctttgtcacttaaaaccctgtcccgacgcggatctcacgtctagacctctgtctttaaagcggatgtagagcgcgttctaaccgttccctaaccattgtgtcaccgcgaaaggcggggctgtgtggaaccgtcccgcacgtgtgcgatgaatctggctccgctgagaacggatccgtgggctgtctggcgcgggctggggcagcagccgagagttagtctacagagctagggcccgagggtggacctgcgtccgcgtctcgtcacgaaaggaagctcttttggagaggcaaaacgtggtcgcccagatccggcgcagctgtagccgtgggcgctgtgcagtgacagccacacccgccacctgtcacg

SEQ ID NO: 135 - DNA, 309 bp, Homo sapiens
  source 1..309: mol_type = genomic DNA
tttctcatcaggatacagttccagcgtcgcacaaacccgaaggaggagtaccaagagatgaaaaacaggcagcccacgccggccatgccggtgactgctctcgcggagacagagaagaaaccgcaggctggccggcagccagggtgagaatccccagcgccaggaactgggtgtaaccccagagctgcgccctcagggcggcgtccacttcggggggcctgcctgggtcacagtcgtttgtgagcgtgaagggcatgccgtagaaatagtcaaatccgtggttcagggggtgtggcagtgatccccgcg

SEQ ID NO: 136 - DNA, 1209 bp, Homo sapiens
  source 1..1209: mol_type = genomic DNA
aaaaaagaaatgagtatcagagtcttatcatcattacagttcttatggtttttaaccgacctttaggagttccaccgacgtgacccaagcagctggtaggagaatctgggctggattcggaaggcgcgctacaggacgcccgctgccccggatcggaaggcgcagcacctcaggccacaatctcagacacgccctcgggtcccgacaggtgttgcccgcctcccacggggctccgggaccccggtccccgggctgccccctgccttctggggcgctggagcgcgcgcagtggcggagctgagccacccagtgcttcgcacgggcgcgctccgcttctccgggttttagcggaagcctgcggggggcggggtaaccgcggaagccggcggccgtgggcgcgcgggttgggggctctcgcgccgctccgggctctccccccccccggctgcggttgccgaagagaggccggaggcggcgcccggctgctccacctggcgcgctgagcaccgcgcggggagcccccgggggcgggcggtggggggcgcggactgcgggaagaaggcacgaggcgtcgccgcagctcggtcaggggccggggccccgccgctcgccgctgcactaacttgccgcttggtctcgcctcccgccgcggctcgctgcgctttgggtggcggggggcggggagagcggggagtcagagggtctgcggtggccgagggagggaccctacgacggggagcccgcccggtgccgctcttcttcccctccccgcccctccgctccccccaccccgtcccttccgccgattccgggaggacgggcgcccgtgacctgcgaacgctgccaagtgacggtccccgagtctgaagcgcccgcgaggaagcgagcgccagcgcgggccgccggcgatgacggccgcgaagcaggagccgcagcccaccccgggggccagggcgagccaggcgcagccggcggaccaggtgagagcggcagccgcggccaggccctcccgggaggggtggctccagtgcgcgctccgcccgcctcccgcttcccaggctgggctcccgcgcctccctcttctcaccctcccccgccccgccccagttccaggctctcctgcttctccacggactctgcgggaagttagagcctctgctgcgctccggggcccggcgagaggatgcgcaaggtggagagccgcgggga

SEQ ID NO: 137 - DNA, 327 bp, Homo sapiens
  source 1..327: mol_type = genomic DNA
aaaattggtgcatttaggaaggaaatgctataaacgcctaattccttctcgttctttggggttgctgagctgacctcgccgcgaggggagagggggaaaaaaacgcgcactaatgattctgtttattagttatatatgtatatattccgtgttcgcttgtacaggaggatttacatggctgtataaagatggctaggggcgccgcgctcttctggggcgctcacggtgacaggctggggttaaaactggctgccccaggagaagcggaggcctggaattaaatacgtttcggcgcactgatttaaataagtttcctgaatatacaaa

SEQ ID NO: 138 - DNA, 629 bp, Homo sapiens
  source 1..629: mol_type = genomic DNA
agagcttccccctacaccaggcacaacccgattctaaagaaatgctgcgcccgcgcctgcagatttcgccttctctctccagatccctctttgccttcctgccagccagccgcgacccgagtcctgagtgaggtgagaagagaaggcctcgcctggcacgttacctttttggtgaaagcctttgccgcaaaattcgcagacgaagggcttgtagcccgcgtggatgcggatatgcgtgttgagcgtggagctgcggttgaacgctttgccgcactggttgcatttatgtggcttttcctaggaaaggggcgagtgcacagtaaggagaggccacctcgggaacacctggaagggacatcccccccacccccactcaaccctagagggtaagggataatctttggccatcgtatccccggtgttatcactaagattctgggggttgcgcgcgggctaggcccgacccgggggcacgtacctgggtgtggataattttgtgcctgcagagcgtgctggcctggcgaaagcctttgccgcagactttgcacacgaacggtctggctccggtgtggaccggcatgtggcgggtgagattatagtgagcgttaaacacctatggaaagacatgg

SEQ ID NO: 139 - DNA, 385 bp, Homo sapiens
  source 1..385: mol_type = genomic DNA
atcttttgctgctgaaatgcagatggtttggacacttcataaagagggcattaataagcccgccgagtgagcgccagggcgggattctcccacaaattcaaggtctgtatctccgcggaagccgtgctatcggtcccccgagtgctgaaggctcccacgaggggcaggccagcccggtgcgccagggaaggagcttgatggtgtgtacggctacgtagtgaacgtggaggtgtgcgcagcggagtcagaggggctagatccccagcatttcccacggggtcccacctagcgcgtggatggagcaacttcatcactccaccgagggcagggaaagaggagtaagcattggagctcaaagaatgagcaatttttcaaagctttctcc

SEQ ID NO: 140 - DNA, 1209 bp, Homo sapiens
  source 1..1209: mol_type = genomic DNA
cgggtggttgaaggcgtagtggtgttccggcttcaggtgggcctcaggcggcaggcccgggtggtggggcgggcccagcaggtgggccgcggcctgctgctgctgcccgggagagggcgccggctctgggggctcagcgccgcagccggcgtccccttcagctctcccaggccccctcgcttgtgctcctggcacggggaggcgctcgagtgaggcgactcggtgcccgccggagtctcggaggccggcccggcggcctccccgagttgagcctgtgaggcctgggctccggcggccgcttcttgccgctgccggcggcgcctgcggcctccttcagcgccagctgcttctcgcacttgaagcgcttctggcggcgcaggtagcagccgttctcgaacatgttgcccgagtcagggtgcagggtccagaaggagcccttgccgggcttgtcgggcgagcggggcaccttcagaaacagtcgttgaaggagagcgagtggcggatggagttctgccagcgctgctggttctgccggtagaaggggaagaggtccatgatccactggtagatctcgctcagcgtcagcatcttgttggggctctgctggatggccatggtgatgagcgagatgtacgagtagggcgctttgcgtgcgtgtagctgcgcctgtaggtcttggggtcgcgggcgcggctcaggcccgcctgcccgtacatggggctcatggagttcatgttggcgtagggggccaggccgcccatggccccggccgcctgccccccgagcgggctcaggctgggactcaagtgcggcccatgcccgccacgccggccgccccggccgagccgcccatgcccgccatggcgcccgcgccgggggacatccccgccagggacgggctcatgccagcgcccacgtacgacgacatgttcatggagcccgcgctcatgttgcccgagccgctgcccatggcggccgccgacatgccatgtacgtgttcatgccgttcatccccaggccggcgttcatgttgctcacggaggagtagccctgcggacagagccccgggagggaggcgacagcgttagcaccgcggctggagggtgcccagacctcccacccaccgcccaggcctccgcgtccggggaggcctccgggggccctttcgtccccgatggcccagtctccggactccgagtctgtttcatt

SEQ ID NO: 141 - DNA, 1209 bp, Homo sapiens
  source 1..1209: mol_type = genomic DNA
acagaggacttctcggggttcccgaattcccagggttaaaacaaacttggtttgtgcggcgacaccgctacccttaccggtccccagatagtcgcggatccccgaagctaagagccagggcaggactcagtcagtgttccaggaacagctgagacacccaggtcaccacgggctcgcgccgctgcggctccgacccggtgcgaaccgccatacgcgccgcgcggtgggccggaggcagtccgcgtactgttgcggaaactctgtctcacaaggtgcgcaacactcgccgcgcgcaacaccaccaggaagttgatgtcggccgggcgcactaggcggtagctgcacgtcgcgcaatcctggctgcattcggcccgcacggtcgccaggagcccggggccgagcaacagcagccaagtgcaaagtgtcaggaaccgcgccatggactgcgaggagagagggacgcgtgcttcggcctgcctgggcgcagaacggggtccctcggcaggaccctcgccgcgacagcctcagcaggggatcgtcgagcaaaagcccgcaggaatgctcctttctggggccccgccctcccggccgacagcttttaggtagacgtggaggcgactcagatcgcctcgcggttcccgggtggcgcggtcgcccccaacgcgaggctgcctggggcacccggctcttttcctgggcgtccgcggcccagggcaagattccggagagaacgccgcagaccaggggcgagagagcgggagccgggggaggcaggacattgactggggtcacaaagaagaaccagggaacgctgtggggaacccatgcaatgaaatgtgaagggaaagagggcgggcagacgtccccaaacccgctcagcaaagacaagcttcgggcaaattcactcacgttgacgctgttcggatggagcaggccaattggaaaagccgggttcagacacgactctagagggaagagaagaaggcagtgtgaggagggcgggcgtcggggagaagcgggcccgggtgtgctggcggacgtagggcctcaccgtcgcggtcctcagcgtctctgcggggtcacgggccaggctgcggggctctgagcgcctgcctcgccgtcccggggcttaacggctgctggagccactttataattaccccaaaccgaaggaggcgcgcgcgccccaatcgccggcgggctgcagct

SEQ ID NO: 142 - DNA, 476 bp, Homo sapiens
  source 1..476: mol_type = genomic DNA
gatctgctcttcaagaggaagcctaaagtggagagtttgtaatcctgacaacgcccagctacctgcgtaccccggactccaactcccagtttgcaacgccaaagggtctaaagggcgaacgggggcgattgcatcctgggagcaagaatcgcgataggtctgatggattgtgggaactgtagttccatgtgcgtgcgatgcactctatggtaggatagacccggggaagagtcatttgccggccaggaagggatacttttgagagcgtcagtgctgcatgccgggatttgtagtctgaggcccgcctcgcattctgggcggggattcatgctccttttcagggcgcccgctcctccccttggtattgccgtcagagatgggcggttggttattgctgcctgatggtggatgaagcagtggaaaagagaattgtatcgttgtgcttgacactaggggtttagttggaattgtgcttc

SEQ ID NO: 143 - DNA, 679 bp, Homo sapiens
  source 1..679: mol_type = genomic DNA
taggattactgcatgttcagctacgacctcatcaacatggctgctggaagtcgtctacaggaaaaagaaagaaatgacactgaaggatcacttccgcttccgttggcgcaagcgctttcattttttcgctaccgtgactaagatggaagcgtttttggggtcgcggtccggactttgggcggggggtccggccccaggacagttttaccgcattccgtccactcccgattccttcatggatccggcgtctgcactttacagaggtccaatcacgcggacccagtaagttctcggcgcttcgtttgcgtagcgggagggaccgtggggcctggtgctgccggctggttttgagagcccgggaaggtgaggcggggaccccgggggcgcgcgaacggcaggggagctcagggcgcggagtcctggagaatgcagaataattggaaggaattatagaaaatcagaagcgcagctagtgcgcggaaagagggcgcagtccatcccccctctcagctcccaccgttctcactctttaggaaccccatggtgaccgggacctcagtcctcggcgttaagttcgagggcggagtggtgattgccgcagacatgctgggatcctacggctccttggctcgtttccgcaacactctcgcattatgcgagtcaacaacagtaccatgct

SEQ ID NO: 144 - DNA, 1209 bp, Homo sapiens
  source 1..1209: mol_type = genomic DNA
tccttagtgacaaggtctctgatctcctgctgccactgcaatagcctctcccatcccgcggggaacggccggagttcttcccttgatctctcccgagtcggcttccgctggggatggatcgcaggtagcgccggcgcggcctggggaagaacagttgcggagcatctgaagcggaaaatccaagcagatgtgaggcgatccgggcccgcctcgttcctcttggggcctgaatttcttccagataagtttcctaatggaacatttctaagaggtggggtacgaggcggcttgctcgcacggcagtgggacagactgcgggtggggacgtactgagaggtccggacctcaatgcgtccgacccgtctccacaccgcccttttccagcccccagtctcctttcattccctactcttcaggctcctttggggccagtgggtgaaccgccatttagaacggtgcctcggactcggggtcgtgcgctccatctctgcctcccccctggggcccgcgaggctggtccgggctttctgagctgggcgttcggctttaggcccaatacctggaccaggaatttcttctccccgcgccagaagggaaagacataggaggtgtcccaatctgcggtcaccgccgatgctcctgacactctagtgagcacctgcccggtacttttccattccaacagagcttccagcttcatactaactatcccacatacggcctgtgggtattagctctaagtgtccttttccgagggcccgaggctccccctccagcagggagagctccgggacggcccccaccaagggttgggttcttccttcacaattccacagaggcatccctgtccttcctacctgggaaacctcgaggtgcggtgcccgtgtacttctggtactttgcgtggtgccatcagggaccccagagccacagctgcgtgtgtgtgtggatgtgtgtgtgtgtgtgcgcgcgcgcgcgtgtacggcgaaggatgtgcttgggggagccgagtacacaacgtctgcttgggcagctgctgggcaggcgttgggcctggaggtatctcacacccacgtatcttccagtcttcaaacacggcattgctctgcctcccgtagcgcgcttcgaacctgcctcgcggacacgtgaacagaggctgccctgggaagataagtgcgctttcccgtaaaatccgggaaatttgccttg

SEQ ID NO: 145 - DNA, 1209 bp, Homo sapiens
  source 1..1209: mol_type = genomic DNA
aagtactgaactgggagcggcagggtttgagtcaaaagagaagggtggaggagcgcgtccggtgccccaaggcttccaggcggagcgcgggcgcgaggtgcgggctccggggcggagcgcacgcccggaggcgggaagtgggcagcagcgcccccgcctggcccctgtgcgccccgacccgacgcctccccgcccgaccaggtgcctcgagagcgcacagctgggagggctctcccggtcgcggcgagactcacgcctgcggtgtgctagggcggcggcgacggtgacgggcgtggggccggcgctgcctcggctgccgggtcgttgcggcgagcgcgcgggccggcctggagtcaccgggctgaaccgccgcgcctgcatcgtgccgcacgccgcggaggcgctcgggtacagaccgcgcgggcgcgcacaaagcggcccggggcggccggcgcggcgcagaccctcggtggcagcgcactccagtcttcccaggctagcggctgcagggagctccggcccgcggcccctccgcctcaagtctgggagctgccggtcccactctgtctttgcctatggggattcgagagtttcccagcggcgcacccaggggcaagagcattgccgtgtgagtagcgccccggcgtgggtgagcgagccggcgggcgggaggccggagcgccggggtagccctttgcagctgggacactgggcagtctgaggctcggggaagggcggcgcgcgcggtggagctgctcgggaagtttcagtcattctttccctcgccactcgccccaacccgcactgccccgagagcccaggatagggcactggggacagctgcccggggaagtgggtgagtggcggcgggaagggaggagcgcgggctctgccgcggcgctcgcccccggagggccccggccgcgctcttgctctcgccctgggatctgctcctcacgaatcccctcctctctgtccatgctcccctgcccacccacctgcccgtggcgcctgcgaccgcagtggcatgaggaggtcaccggacgtcagcccccggagactgtccgacatcagcccccagctccggcagctcaagtacttggtggtggacgaggcgattaaggaggatctgaaatggtcgcgctccgtggaggatctcccagcgggccggtggggctcacgtccatcgaggagcggatcctgcgcatc

SEQ ID NO: 146 - DNA, 475 bp, Homo sapiens
  source 1..475: mol_type = genomic DNA
tgcatcaccaccttggttttgttttattttgcttcttggtcaagaaaggaggggagaacccagcgcacccctccccccctttttttaaacgcgtgatgaagacagaaggctccggggtgacgaatttgccgatggcagatgttttgggggaacgccgggactgagagactccacgcaggcgaattcccgtttggggcttttttttcctccctcttttccccttgccccctctgcagccggaggaggagatgttgaggggaggaggccagccagtgtgaccggcgctaggaaatgacccggaaccccgttggaagcgcagcagcgggagctaggggcgggggcggaggaggacacgaactggaagggggttcacggtcaaactgaaatggatttgcacgttggggagctggcggcggcggctgctgggcctccgccttcttttctacgtgaaatcagtgaggtgagacttccagac

SEQ ID NO: 147 - DNA, 551 bp, Homo sapiens
  source 1..551: mol_type = genomic DNA
ttggggttgtagtctatctgggtcgtataggaatactcattctcgaacagcagcctggggacgatctgtgtgttggtgtgggtgtcgaaagcgtaggagatgttggcattccgctggttgtagctatcacggcatacagcaccccacagatgacgaagcagttgccgtagaaattcctccggagccccgtgcgccatgtggtctccttctgtgtgctcaggtccgcggcattgagcttgctcaggacaatgacctcctggctgaagccctcatcgtccagggccgggtagatgagccatggccattctcgtccacagcaaagtccacgtctgagtggccctgccatcgccagggggtggcctcctcgtaggccacgtcatgcagcatggcccaggcagccacgtagcgctgcttcaggtcgtacttgatgatgttgcgggtgaaggcgcgattgtagtagaaggcgccatttataccacgtggcctgtgccgatccagctgtacgggagcttgtaggaattgctccagcgacctgcaggtggggagaaaac

SEQ ID NO: 148 - DNA, 1209 bp, Homo sapiens
  source 1..1209: mol_type = genomic DNA
tcacaggccagctggaagtgggcagaaaccctgtgggtcccagaccctccccaatcggccgctgctcccacctctcccgccccaatttttcagcagctcgattcctgcggatcctacatccgggaagaagcagacgggccctcctcccctccctcgcctggcgcgcggcgcctgggttctcatcaccacgggcccagtgctcaccgtgcgcggggctgtaggagacgcgcgcccgcgcgtgagcggggttggcatagtaggggttgccctgcgagtcgaggtgattgaagaagacgtcccctcgtctggaggcagcagctgcgcgggttccatgtagttgtgcgccaggcccgggtggtgtgagtcggggtgctgcgcattcagcacggccgggtgcgccatccagcgcggctgctcgggcgccacctccatggccggcggcggcggctcagggtctgggtgcagacggcacggccctgcgcgaggaagggggagtgaggcgtgccgccagcgcctgacaccccccaaagtcccaccacgaggtgtcccgcacgccacggagccccagcccagatccggcgagaaagagcaccagtcccgggtgggaggaaagcccaaggctcaaaacgaaaggaaggcggggagggggttcagccacgcacactcacgtggtgacccgcggctccagaatcacacacccgtgcacatggggtcacgcccggggacgggtcccgacaccagtgaccccaacaaacgcacagagcagcacttcagtcagacactcacactgagcccccccgcccggtagacaaacaatgaacacagactcaaaagttggagacaggcgcccgggcacccagtgtggcacttgatcccagcgacacgcacacacccacacttggcgccagatacacatactgatctcaaccccgaaaacatgcacacgcagccccctgagcgcagtactaagcggcacaatcaggaccttcaacaaagcacaccaaagcagtcgcccgcagcctggccccccgccctaagtccccccagagtcccctcaaagctaggagcgccccaggcccccagccggctctcaaaccccaaacttacacacgcagccgtggggaggggagggactcggcctctgagagtgaaggagttcggcgggagccccgagggcgacgggcccagggacagcacgtccggaggctg

SEQ ID NO: 149 - DNA, 601 bp, Homo sapiens
  source 1..601: mol_type = genomic DNA
gtctgatctgtgaagggtagggccagcaggcagcaccaaagttcccgtatgcgcgttttcagtcttcatttaggtccgaattcccggcatataagaatactaccgtcgcttgtttttcagatttttgggctattttcgttggtgtgttggtcatgtctggtcgcggcaaaggcggaaagggactgggtaaaggaggcgctaagcgtcaccgtaaggtcctgcgagataacatccagggcattaccaagcctgccatccggcgccttgctcgtcgcgggggtgtcaagcgcatttctggttcatctacgaggagactcgcggggttctgaaggtgtttctggaaaacgtgattcgtgatgctgtgacttacacggagcacgccaaacgcaagacagtgacagcgatggatgtggtctacgcgctgaagagacagggacgcactctttacggcttcggcggctaatgctaccgttaaacgactcagcatctcgacttcccaaatcaaaggcccttttcagggccgcccacagttttccgcaaaagagctcatgacttgttagacgattggttggtctctttataagttaattgttcctgtcaa

SEQ ID NO: 150 - DNA, 22 bp, synthetic construct
  misc_feature 1..22: note = primer
  source 1..22: mol_type = other DNA
gggtttttagaggttaggggaa

SEQ ID NO: 151 - DNA, 25 bp, synthetic construct
  misc_feature 1..25: note = primer
  source 1..25: mol_type = other DNA
caactaaataaccaaacctctcctc

SEQ ID NO: 152 - DNA, 24 bp, synthetic construct
  misc_feature 1..24: note = probe
  source 1..24: mol_type = other DNA
ggagaggggggttatgatgttagg

SEQ ID NO: 153 - DNA, 20 bp, synthetic construct
  misc_feature 1..20: note = probe
  source 1..20: mol_type = other DNA
gtttgtttggttttagagaa

SEQ ID NO: 154 - DNA, 8077 bp, Homo sapiens
  source 1..8077: mol_type = genomic DNA
cgggcaggcccaagctgcgatgtggagaattcgatgtccgagcgacctcctcggaggagtgggtcgagttaaatataaccgcgcgaatggaatggcgctaaaaataaggcagcagctggcctgtccacagccctgtcccgggaggggcgggggccccagtggtcttgggcaggaaggccgcgtccggcccaggggcgagaaggctgcggcgtccgcagccagggctggaaggcctgggaggccgcgctctgtgggccccggggcctccattcgggctgggtcgcgggcctggacggggactgtccagaggcatccgaaagccaggccaacttgcctggacgtaacaagacggaagggctgggcgctgaggtcctgccagcccggccgccagagggagctgagcgccagaggaggacaagccgaacccttcaggaggccgggcgtctccggagaccgaagcgccggaggacccgaggaggtctgccccgcgcgctgctctggagactcccggggcgggtggcgctcggcctttccgctcccttccttcccacaagtcccttcccgcgcgcgccccacggccctgcccgccctcccgcgtcagcgccccaaccgtcaagccagcaattgaaacgtttccaaaacggtctatttatttgctcccaataaatcgatcggcggtgattaaagaatcgatgtggcctgggtgggcgagtcgcttgaggggagggattgggggctttcgcccggcgcctgcagggaggccgagggcgggcgcgggcctgagggaggcgtgtcccgcccgggccacacccgaggacccgacacctgggctggcaggccccggcaggcagcgttccctccggcggagaggggcgcgcgcccgccgcctgctttcctcggcccctctcgcctttctcgcgcgccggggaggctgtggccgccagtggctgcggagctgctcagaggcttttgttgctcctcggccggctgaatggggattttgtaaagcgggacagataaaaatgagcagcatcatattgtttgacagaatgatctcgcatgatgaagtgtcggctccgaagggggtgaaaatggtgaattcctaaaaacccagccctgggctcctcctcgagctgccggtagcctggagggacccagcggacagccgggcctggccgcatcgctccaaacggtgtcagaaagactccggctttcaatgccaagtcatttttaagccccgatcctgtccaggacctttctcctcgtggatgaaaagaacaattttcgagagaaaggctcgtttttattaaatccgacatgctgctgataactccatgctaatgtgaaataattaacataatagccataattaaaagcacgctaacaatgccataaatttatcacacaattttactagctttctgcccctaactgctctctcatcgttaattaaacgtgttgccttttacagaatggatgtttatatatttccaatataaataaattcgaaaccatcctctctctcttcctctttctctcctcctttccttttggtctctcgccatttacaggcacgccttggcgtggaccctgagtggcagacatcttgaaaataaatgaagttttgagatgcaaatccaaacaagaacattaaaatagcctctttttttccaccccgaaaagatccggagaggtatacaagggggtagtggtgggtaagagagttgaaaatcccccgctttgggaaatggaagtaatctgggtgggttggggccttgggtaccacctctgccctttcccaccttccttggtggcggccatccagacaaagaggccggtaatagtttaacaaatctatgaagattttcaagaagcagcagactttgattgttgcgggcgcgggggtgttggggagaaaggaggggaatttttctaatagtcccacccacgttttgctccctcttggacaaagagtaactactcttggtgggggacgcgcccttcactccgcggaacctggtcccaactccccgtattgtaagaaaagtgcacccgcgcgcgggcatgatgattctatctcacatcgcgccaacgacttattcaagccactggcactgtctctgacttaaaagaggagaaaagaggcatatgggttcacttgggcctggtgaggggtaggtgggcaattcccgccttccgcactctaaccgtgcccctcctccagtgttgaccacctaagaacccaaaatgagctgtaattaatttccctttctccatcataaatttttctatccatttcttcccccccatccccccactggacgcacacactaaatctcccctcccctggagacgtctcaatttccttcctatcgatccggactccattcttcttgcctcctgttgctagaacctagatccccactccccgcacccctcattcccaccgcgtccaggtggctttcccagcggggtaccatgtactctgcccgctccagaggaaccgaaggggtttcattccattctcctttggttgaaacatttcaaacatttgagcaggtgaggcagctggctgccatcttcctttttaaatctctcctgggaagttcgcttgttgagactcaaagagtcactcaaactcataattgcgtgtgtgtgtctactcattctccctctatctctccaataaccctttgagactcagaaactttttatccacatacaccctttatcacattttcttccccccactacatgtgtctcactttctctctgtatctgtctcgcttcttccgtctctgtcctacagcttggcggtaactgacgacctgtgagcttttagctgcaaactgcaactacgcggcaaacaatttatttagcccgacatctagccggtctccggcaggaccctgcaccgcgtcgggatcggacccttccgctggggcggcctcctgcgtcaaggccagcaggaaccttcctgtcgccctccccggccgccgcttcgcctccttcccgcccccggaggttgtgcaggcgctatggtccgcctggagggagaaagccggcggccggttcctgagccgagagcggccgcggaaaaatcctctgcctccgctggaaatcgatattaggccggcgcgggcgcgggacgtcggggccgcagccagtaggttgtgcacgtctcatcatttagctaatcgagtcgaaaagtttctgtaagggccggacccagcatcagatggtaacactgattgaacaagagattagcacaatagatctctaaccgaggggaagcgttgcttttcacgctacgcgccgtaattaatggtatgaatcaattaatttgacttttattgtgtcgaaggaaaaaagcgcaacaaatggaaccggcagctgggagttgttcgtcctccacccccttccccagggaggttccaaggagacaccggggaatggacggatcaggctgggccgtggcagagggagggtaggaggcagcgaccagcagcgtggagggagtccagagagctagcctctgcggacggcggaatcgaaattaggctcatttggagactacttcgagaccggtgaggggagccctgtagccaccatcctccggcgcgcatccacacatactagtccacgcgggcccagccaccaaggccgcggcagggccagcgctgcgccccgggcccctgcctttagggctgggcaacccaagcagagcaaaggaggttcctgaatgtgtaaatttccgctttttagctttttttttttttttttttggaccttccgacacttcggttgctgaggcagttgcagacgcgacctctgcagtcctgggcgatggccagccagctcagctcgggtcggtttcgcggaaagctgtctagacggcattgtaaacggttcggagcctgcgggccacaaagctgtggagctacggaaatcaactctgagatgcgttttagggccgtgtgcaacctcgggatcatttagataaagaaaaactgtggaggttggcgggcgtctcaggatagtgtcaccaccccctaccctgctcccagcctcagatgagtagtgttatatcctgggaaactgtctaatggggatgaaagtcaatctgtgtgtctcaatgcctgtaatgaagcaagtttacagatttttaaatttttatttttattttattgaattatttttggtgtgtctaggccaaggaaagaggagatcgtgggtggggaaacagactgagggaatcagaagcaccactgtccatccggaattaaatccacatcccagcatcttctgcaaatatttcactaattatttcctctcggaactcctcccctcgtgctccttcctctggtgaggccggcgctcccctcccaggccgcagcggacagacagggattgggttccgtgtgcctgccacaccaggcaggctcttgcggctcccaactaggcggcctaaatgagggaggaaagaggaggcgcatcgctgattcaccgcgtcaagagcactgactttccttggaggtgtgaggtccacgcaccccagccacgcacttgggggtcggtttgcggtgcctccccctccagtcccagtgaaatccccacagtttttcctactatcactgacttgccttgcactccgcgtgcattggccacacatcctcgcctcctccacccgctccgccgccggttttcttggaagttaaatcttggaggatttgtccacaccttaagagaagaaaatccacgttagctggcagcaacggagatcccagcatgctggcatgcccaagtctgcccaggttcccccaaggccatgcccgccgcccgggaagtcactgcccgcacccctcacgtttcttcagccgcccctgggcgctgcgtctaacctgaagacaccaggcctcttcccggatccactcgacttacccaggccgctgccaatcccagctccttccccagcgcctcatttccgattttttcatatgctaagtcgtttaacaactccaagtagccagttatggcttctttatttataggttccctgctattttacgtcgtttttatttctctcggcaactattctagtagattaatcaatagccattttctgaccttcgggaaccccagctgatgctttttgtggccgcacgaaaaaatacatacaggaaaacacgcccgcatcaagccgggaaagagcaggtaggacctgagtggtttggttgggggagggggaaaaagacatctcagcaggtgtcttccccggaatgagcactgaggccagaggggaatctgaaatctaattagcaggagggagccgggtgcgctgctcttactctttaaagctaaaaacaatgaaacaaaaagcaaaacagagactaagttttgctttttaaaacacgatatgggaacctcgttctaggtcgcccagtccctgtctaaggagtgtgacaaagtgggggggagaagggcggaagggagagggggcggggaaggcagggcagcgacagtcgcacagtcccgcggacgctcccaggcccacgccctgactcgctcacacccacccacactcacacccacccgctccctgggccccagggcccggatccagcctgggtgggggggtctccgggcgggccgcagcgccctccgtgccccggggatgctggcgcacagtgcggagcggagttgcgcgtctctcgtccctttgttgacaattccctgaaccaacttgagtttggccggctcggccgcggccctgacgtcacgcacggtcacgtggccccgcctcccgctggatctttaagtagaaagtaatctatcaggccagtccttaaaacgggactttcgactaccggggcttcggcgtccctgacacccagccccctgcccccccgctactgtccctgcccgcgccctcccgagctgctcggcgcccggcgtcccgcgcccgcctggaccgctcctgcgccccacgccagggccagaggccgaggaaggcgggctaagtgagggggcgcggcgtggagaaccgccggggccgggagcggtagcgagcgcctagtaccgagcgccagggacggcaggagttcgcggagcgcggccgctgggggcggacggcagagcccgcgccacgcgatgcggggccgccgagtgtgagctgagcccagcgggccccaagccacctgcggccccctcccctctccctgccccccatctttcgggggcactcaaaccctcttcccctgagctccgtggcagcccccgaacaccctcatcgcccgctgccccctccccgccgccgctaccaaccccgaggagggatgaccctctccggcggcggcagcgccagcgacatgtccggccagacggtgctgacggccgaggacgtggacatcgatgtggtgggcgagggcgacgacgggctggaagagaaggacagcgacgcaggttgcgatagccccgcggggccgccggagctgcgcctggacgaggcggacgaggtgcccccggcggcaccccatcacggacagcctcagccgccccaccagcagcccctgacattgcccaaggaggcggccggagccggggccggaccggggggcgacgtgggcgcgccggaggcggacggctgcaagggcggtgttggcggcgaggagggcggcgcgagcggcggcgggcctggcgcgggcagcggttcggcgggaggcctggccccgagcaagcccaagaacagcctagtgaagccgccttactcgtacatcgcgctcatcaccatggccatcctgcagagcccgcagaagaagctgaccctgagcggcatctgcgagttcatcagcaaccgcttcccctactacagggagaagttccccgcctggcagaacagcatccgccacaacctctcactcaacgactgcttcgtcaagatcccccgcgagccgggcaacccgggcaagggcaactactggaccctggacccgcagtccgaggacatgttcgacaacggcagcttcctgcggcgccggaaacgcttcaagcgccaccagcaggagcacctgcgcgagcagacggcgctcatgatgcagagcttcggcgcttacagcctggcggcggcggccggcgccgcgggaccctacggccgcccctacggcctgcaccctgcggcggcggccggtgcctattcgcacccggcagcggcggcggccgcggctgctgcggcggcgctccagtacccgtacgcgctgccgccggtggcaccggtgctgcctcccgctgtgccgctgctgccctcgggcgagctgggccgcaaagcggccgccttcggctcacagctcggcccgggcctgcagctgcagctcaatagcctgggcgccgccgcggccgctgcgggcacagcgggcgccgcgggcaccaccgcgtcgctcatcaagtccgagccaagcgcgcggccgtcgttcagcatcgagaacatcataggtgggggccccgcggctcctgggggctcggcggtgggcgctggggtcgccggcggcactgggggttcagggggcggcagcacggcgcagtcgtttctgcggccacccgggaccgtgcagtcggcagcgctcatggccacccaccaaccgctgtcgctgagccggacgactgccaccatcgcgcccattcttagcgtgccactctccggacagtttctgcagcccgcagcctcggccgccgccgctgctgcggccgccgctcaagccaaatggccggcgcaatagggacgcgccaatggccgggacccagggtccggcggcggcctcgagcaacaaatgcacctccaggctgcgcgccctgtcccaagcccggtcccggtcccgctgcccaatcctggactctgcctctccccaatttcctttcccctgagcccccaacgcctaccttccgcggcctccatcccctcgcgcacacctaagctggtcgagcaaactcaccgcgcgcccgccggggatagctttccatacaggtaaaaccgaaaaccgaattttccaaaaatgcaccccgacggcgcctgctcttagtaccg

SEQ ID NO: 155 - DNA, 348 bp, Homo sapiens
  source 1..348: mol_type = genomic DNA
cgcgtctgctaccagatgcggctccgggggctccatggtgactgagataggagaagaaggcgccgtccctacggtatcaatctccgaacagggagatggggtggcctgactcctaaaatccgcggtcctggcctcaccgagcgggcggaaatctccattcatcatgcctgggctgcctgaactggcactggacaaaatcgtgtctattccaaaactcgacccgctggccccttccattgtcattgctacatgaggtccacgccactccgccgttcagcagccgccccgaaccagcgaagaaagctatcgatcgtaaaacaaaataaacaccaaacaatgttgccgccg

SEQ ID NO: 156 - DNA, 325 bp, Homo sapiens
  source 1..325: mol_type = genomic DNA
cgagaggcccggccaccggctcgggagctgggccggtcgctttgggagcggatgaggaaggttaggagaagcagcgagatagatcccaattttacaattctattttctttcggtagggtctcggcgtcctgggccacgttgagagcgaacgtgggccgagcggaggacacagagtaaaaagcgacgcccgctgtatacataaatccgcacccgctgcccgcccgggtactgcctgctctggcttccgctctcttccgaggctgggcaagtccaaaagttcccgaaaggggggttcaaagaggggcgctcagtgcacgtgatttcg

SEQ ID NO: 157 - DNA, 431 bp, Homo sapiens
  source 1..431: mol_type = genomic DNA
cgcggaagagggcggaaggagaacccctggactggtcctcaccgagcactacaggtggcagagccgtgggggacccgtgggttatgagattcagcggtgaggggtagcggtgtggataaaagtagaggcctgacctgcttgaacgtccgcagcagcgagcccagcatgaaggccgccatgttgtccgctacaaactacaaaccgctgaaactttattgacactgccgaatagcgcaggacacgaggggcggggcctgggccgcctggggcggagctgggaggtgggtgggaatgtggcctacaggaaagcggggttagttttgggatcggagctctcgcgatatttcacggaacttgggcaaatgacaaatctgaaggcgccgcgttgatgctgatggcgcagccagacccgccccgtgtgttgcagcccg

SEQ ID NO: 158 - DNA, 703 bp, Homo sapiens
  source 1..703: mol_type = genomic DNA
cgtctggccaaaggcgcccagctcaacggccgcgacgcggtaaggaagggctccgccgcggcggtagccgggcacccctgcgccctctgttaaaccaagccgactccctggccggtggagaaccggagcggcgggtcgtagagacgtccgcttgagggtggcgggtgagttttctcccgtccacctatgcctacagggctaagaaacggtcgtttttctgaatcgaaatgtctgcagctggctatcctccgtcgcagctgattccgaaagagcagggagaggaactgggaggaggtgggatggggggtggtggcattctcttctacagacctcaaggttcccttgattccggggcaggcgtctccccggcgaggatccgcagctccgagggcaagctgggcataagcaataggaggacggcgcgctgccgaggcgtccgagccaagcaggagcccaggtggccttagtctctgggcctgatgaccggacctgtggagtagattccgacgcgactgggtcatttccagttctctagacgctcggggcttgggacccctaaccgagagaatctcagggtttctggcatcccgactcagtccctctaaggagacagcactacgtttagcgccaggacccggcgggtgtcatgtgtagggggaaatcaaataaaacatacggaggagcgcggcacccagtcgaaacg

SEQ ID NO: 159 - DNA, 416 bp, Homo sapiens
  source 1..416: mol_type = genomic DNA
cggcgggggtcgctccaaagacttgtatttcgcgtttgcctccgggagctgggagtaaggccttggatggcgccgacgcggttgcgaggaagctgaggcctgggagagcaaggggcgcgcaggcgaagttgcaacttgcactccagccgcgggcctggcggagaaagggaggctcgcggcgccgcgaggagtcggcgggcctcggggcctcgctttcgccgcatctgcccagcgctccgggccttgaatctcggcagatgcgagttgtgggcacctagggaaccctgaggactcgcattcccccgggtctgtatcccgtgcccaccccggagcgtcgcaaaccttggaaagggtgaaagctgataggggagtcttatttcttaagagagagggtgagggcgcgcggcgctgcctcg

SEQ ID NO: 160 - DNA, 332 bp, Homo sapiens
  source 1..332: mol_type = genomic DNA
cggaagaggtggaccggctgcggacccaggtactgtgcagaacgcggcgcaggtgggagtccttgggcgggcgtctacaggtggatcttttatgcacctcaacaggccggctttctcacggatcacatcggttagcagtaagctgggagggaggtcccgaagactgtgctgggcacagtgcaggacccagagaagcggagccatgggcccgtcctggtgagggagttaaagtccgtgcaggtgaacagaccccggcccgcgaatcaacgaaccacaccgagtgccaagttgcgccgtgcaggctgcggagggtgcgagagcagggagaagcg

SEQ ID NO: 161 - DNA, 1475 bp, Homo sapiens
  source 1..1475: mol_type = genomic DNA
cgaacgcaacatcaagggcgaaaagaggctgcacaaatcacgtagaaaattagaccgcctctttccatggcgataatcgaattagcctcaaattaaacgtgaaattggccctctccggctgtggagacaaagcccctattgtcctgctcgggtagcggcagcagcagtgcccaccccgccccggccagcctggtcggtccccgccgctgggaggatgaggtccgggctggacccgggaagagcctcagctggagaggacgtggacgcgcccgaggctcggctccctgggcggctgcggggctccgggcaggaggagagggcgggcgaagcagagggagcgggcgggagtggggagggaggagtgcggagggcggagggcgtgcgggagggatcccgggagtgcgcttgtgtgtgtgtgcgcgcgcgcccgccccgcgcgctcctctccccgcccctcgccccctccctgcgagcgccctcctctcctcctccccccgccccccgcgctcccgcccagcccccggaatcagtgccgctgttgccgtgcaggctgcggattcctccagtccctccctcggccgcctctcctcccggagcgagcgcgcagccctgcgcagcagcgcccactggtcccgtcctgtgagccccggccccagccgcggacagacccgcggagtcgcctcccggcccacccgcccggccgccgaggagcgggaggaggacgggaccccggcgcccccaccccatccccgggaggtaggtagggggcccgaggcagggagatgccaactcactggggctgcctcccgggtgggctccgcggcggcggacgggcgggaggaggggcgcgggcccagggccggaggacgccgctgcccgcctgccctgcgcccggggagccgcgtgggggcagggaggctgcaaagcccgggcgggggcggcgcggggagcccgggtcgcgggtgctcattggtactcggatgatgcgggacgaggggcggcaggctctgcggtgaggcggcagcagacccgccgtgcgcaccgctgccgtccggccccagccccggcgcggcgcggcgattcctcctccctgcctccgccggcgcctccctgcagccgggcgcggcggcccctgacgcggaggcccctggcgcggaggtgggtgcggagcggacagcggacagccggagggtctattttcaggtgccctctctgtggccccgcgggtggggagcgggggcggcggtgacagggccgggctctgcgcgtagcggtgcaggggatgcggccaggcgtggtcccggctgcgccgcgctgcagctgagccggaggcggcggccccgcgcccccacctccgccccatccctgcgcttgctaccctcgtgcgcccgccccccccccgccctttctgccgtccccactctcccagggctgggcgtgagccgcctccgggcgccgagccgccgctgcgcccg

SEQ ID NO: 162 - DNA, 860 bp, Homo sapiens
  source 1..860: mol_type = genomic DNA
cgcgttcccccgcggagtggctctcgagtgcggggaggtgttgcggaggggagtggacttagggaaggggcggcaaaagggcaaagggagaaatggcgtgtgtgtgcgtgtcaaggaatggagagggcagggcgcttgggagcagggcgcgaggccaggctctgttgggccccggctcacggcgccccttctctctgtctgtacctgcgtgtgttgccgtcggcggcggggccgcagccagcgacgtgccccaggacgggctgcttctgcacggccccttcgcacgcaagcccaagcggatccgcacggccttctcgccctcgcagctgctgcggctggagcgcgccttcgagaagaaccactacgtggtgggcgccgagcggaagcagctggccggcagtctcagcctctccgagacgcaggtaatcacccccggtcgcggcctgccctgcgcccggagcccgggtggaggtgagggtgcgcgggtgcaggagaggccctgagcccgccccagcccagccctgctgggttccaaaaggcccccattccccgcggcgctgcggtcaagcccgtctttagagcctcttcctcgagactgcgtgcagcctgctgagcccgcaggacttttgtcaagcgctaaagacctagcaggaggcagagtaaatgcaaactgtatcccgagcccggctcccaaagctcctcacggggggaccaggttccctggaggaagcgggtcgcctcgggagcgggcagcgcaggcagcaccgaggccactggagctggctccagccctggcattcctgcagcccttttcccgccactgtgtcggggcgctcatagtcctgcggggagccggtccg

SEQ ID NO: 163 - DNA, 2637 bp, Homo sapiens
  source 1..2637: mol_type = genomic DNA
cgggggtcgcgttggaaccccacaggaaaaaggcgcggaaagccgccgggcattttccggggttccatagatgtccccagtgtcctagtccgtgcatcagctcgcgcactcggagggactctaggcagggggagggccccgcggccagtatgtgcgtccgaggctttcccgcagggggcagtgccgcccgcccgcgcgccgatacggtgggagggggtgggaacctgcgcggagttctggaggttctttgggagaaagttaggggatgcggaggggtgggcgcaagacttccaggactccagggaggccgtggggagggccgccgagggtgcagtgtgaggcgcaggagggggttgggggcggtgcacgttgcagggagacgcagcccctggaagatgcgagtgtgaacgtgtgagtgtgagtgcgtgtgtatgtgtgtgtgtgcgcgcgcaccgcagctctccgggttccgcgaggcgcgcgggtgtcagcttgcagccggggctcctccctccggcccccctgcccagcccggcggtccctcctccctccctccccgctcgcccctccccggcgggccaggggctgggacgccccggcggagcaggcggcggcggtggcgagttggggagccctaggctcggcgctgccggaggggcccgagccgagccgcctgcgccccggccgggcagcgccgggcccgcttcccgcggggccacgccctgtcaaactttgttgcggcggctagcgcagcgggcccgcaagcgggcgggaggggcgccgggccgggccgggcagggcgcgggcggctaggggctccgagagcggcggccccggcccgcggccccaccatgccccagctcggcggcgggggcggcggcggcggcggcggcagcgggggaggcggcggctccagcgccggggcggccggcggaggggacgacctcggggcgaacgacgagctgatccccttccaggacgaggggggcgaggagcaggagccgagcagcgatagcgcctcggcgcagcgggacctagacgaggtcaagtcgtccctggtcaacgagtcggagaaccagagcagcagctcggactcggaggtaaggaagcaccgcggccacccccgggggatcccggccctgcgtccgctcacccgctcttgcctttgtgtctcctccgcaggcggagaggcgcccgcagcccgtccgggacactttccagaagccgcgggactatttcgccgaaggtatgtgcccgctgggacagccccccactctcgattcccgctgcgctccgctgctcagcccgggcggcccaccgtcccccttgcttgggtggacgcacccttgccctccgcctttattggcggcagcccccgtggggcgcgcgtggggggcgctggggtccccagctcccgcctcgagccccctgccgcggcgctgtcagtcccgggggcctgggcctcacctcgccttggtcttgttcgcagtgagaaggcctcaggacagcgcgttctttaaaggacccccgtaccctgggtaccccttcctgatgatcccggacctgagcagcccgtacctctccaacggacccctgtctcccggaggagcgcgcaccgtgagtgcccgtcgggcgcgccggggagggtgggaggccgcggcccgcaggatgcgcccccgggcttggccatggagtgggggatggggccttctgcgccgatcccaagcagaacttgtttgcggagttgaactactctctggcggccgagcgcgaggctgcgctggccagtgcctggatgaaagtaaagttactttaacttttcccctcttgcgggttgaggttttggagtccacctctgggatcttccttggcctccagaattcttcgcctgcaccgaaggaaacttggatttgtgcccgctttgggggggtctcgctttccttcttggaaatcggtcagctttctctggcagtggggcaaggggcctagggagctgggttggcgacgttgtcctccgactccgggttcactgggcggctgcaggctggttcctaagaaacccagttttcgtggcgggttattcacagcccccgttcccacccccagccctcgcaccggggcctcagcttttctgcggagctagctccgaattttaaactcgcgtagatgatttcgaggcgacccaaggcattcttcaagttgaggaacttggctttttcccccttttgtcgcctgcttttctcatttaaagcgagcgctgcgacactttaaacctgttaatgggcgcgtattgtgtgctgcccgctggcatttagagcggtgattaagcaaattgaccgggccccagacgacgtgcaaatgagggcggattccttcgccccctctctctggctctaaactcccgccccccgcgttgcgaggggcgcagctgggggctggcgaggcctttgtgtcccccaagccgccactccacccctaggctccctgcccaggtgctgggtccgatgacagatgttgggtgaacgcctacctactgtgtgccaggttcccgcgtgctgctgcttgggcgcgatgtgaatcgtagtagttctttgcttcccgcccagattcccattgcctgccacttcgctgtcgacg

SEQ ID NO: 164 - DNA, 2534 bp, Homo sapiens
  source 1..2534: mol_type = genomic DNA
cgaggaggctgcagggacgcgcatggaagagccggtgcgtgggagggtttgcgggggggacatcgcgccccctaggggtgaccccagtgggtcccgtgtgctctccgcggagccggcggagccttgtcctctgcacccggcgcgcagcggccccttaaacagtggaaccgtgaggccgctctaagccgaagggctggaatctgggtttctcgggttttattttagaccattcggcaccaagcccgagctcccccgccgcaccgcttccagtcccctttctttccatagagcgacccgaagccggcggtggcgcagggagccgagtctgatgagctcgcgggcggctgaaggccggcttccctgtggggaacgcgccacctgtcggcgccagtgagaactgcgtctgtgtggcgccctcggggtattcggggctgcggggagatgtgtgcctgaagccctgcgcttgcggtggggacgtccggcctctttcctggcaattgacccctgaggcgggagagacaacggaattcccacaaagggatccttctcgggatctccccacctcaagacagctaaagctggaggaaaagcccctccggggggtggggggtgcgggtttgccctgcgattccgaaagcagaaaatacccgagccacacagggacgggcgccgcgttggtagtcggggctacgttcctactccctctacctcccccgcgctgtgtgaccctgggcggaaccccgctgctctctgggcctcagtgttcttattcgtaaactgagggcgttggatgagattggtcctctcccaactctgaccttgaaactgatactgaatctgagcagcgtctgtagacacctgtgccttgccttctatttctagccttgaataaatcctggacttttatgtgccatttatatcctaatctcatatatatttaatgtataactgctgccattattgttttctcaattgtctaggttttcatttggatggggttaggatggtccaaattatcccgataagtgcccattaacttaaacctttttaaaaaatgaaaccagtaaaacttcattcactttgcagtgtggacactgctggagagcacccatgtcgtgggtccagcgaggacacaaggaggggcttagagacatgcgggaggcttagatgagaagacagcacccgggcagcggtcagtgttagagagaggacccgtaagaagggccgaggctagagggagagcgaagactgagccaacgacgcacctgagccctggggtgggggtggagacgtggctcctaacccaaatctccctgccaggcagtgtccgacgagcatcgacggcaggcgtcgagaccagtgcagggtagctcagacctcaagccacgcttgacctttccatgaaatgaataaaactcgaaagccagggaaaggggacagtactttgatccggagatcgcttataacctctgcttggagttccgagttcgtgcggctcaagggaggctacagtccagcaagctctgggctccaagcgtggggacggcagcccccaagcttggcgcacccctcgggaagccccggaacggtcctcgccagacatagccggctgtcctggtccttagcttcaggctggcggcgcaaggccagagcggctgccttctaggcacctgggtggaggtctcgcatagcattccctgagaagcgaaactgcccttggggccgcagcgagcctgccacatcgaactggagaccctctgctttcgggatagatgggacgtttctgctctgtccttcttggagtcccggaatcgttctggggccgcgtgctgcctggaggcggtgaatttcagggtcttgagaagccgcgcacacacgggattctgggcgagcgtcccgtctcttaattcctattaagagacgggaaaatcgagggactggaggtcccatcattgtcgcgtgagcagcctcctgaacaccaagcgagacctgagggttccgctggggcctcgccctgacacccgggccctccgtgtggtcgagagtttgcgcccgctcccgctagggcagcgaggtcccacttgcggccggctggggcatggtggcaccggttgtctactccccacttgtgacaccgacagcttccaactcctcagacccaccccgtggaattctggactttgtgagggccgccggggtcctggccctggggtcagctgccatctgactaagccaggacggcggagctccaggccttgctccagcactgccggtgcgtcggggcccgcggagagcccagggcgggagctgtgggctgagccgggtggccgcgtggacacagatgcccggccggactgagcggcagccaagactctccgtccatcccgccgctggactcgactctcccagacccgccacggaacccagatttgagcacgcaagataaagacgccagaggcgagtgcgcggcggagaactggccgcgacacgggaagcttctggggcgcagaacgctggctccgactcgcgcggcg

SEQ ID NO: 165 - DNA, 396 bp, Homo sapiens
  source 1..396: mol_type = genomic DNA
cgacggcgcgcttgtggcccggccggagcttgcgtgcgcgttctgacggctgggtgctgtgttacaggtcggcgcagttcgagcacacggttctgatcacgtcgaggggcgcgcagatcctgaccaaactaccccatgaggcctgaggagccgcccgaaggtcgcggtgacctggtgcctttttaaataaattgctgaaatttggctggagaacttttagaagaaacagggaaatgaccggtggtgcggtaacctgcgtggctcctgatagcgtttggaagaacgcgggggagactgaagagcaactgggaactcggatctgaagccctgctggggtcgcgcggctttggaaaaacaaatcctggccctggactcggtttcccagcgcggtcaacg

SEQ ID NO: 166 - DNA, 3176 bp, Homo sapiens
  source 1..3176: mol_type = genomic DNA
cgccctcgccccccccccgcaagtatcccccactactcccccccacccccgcggccctagctagctgacttgactggcacgcgccgggagcccgggctcgggcccctcggagcgtctgattggctgcggggcagctccggtctgctctgcctgcgccctcattgggcgagaggcgcagccagcggcacttcaaagcgggtgctcctcgcacttaggctgagtttagccggcgggagcctggagtccgctcggcacgagcgcggggacgcgggagccgcgcgggacccaagcagtttttccgagcagccgccaggctcagccccgctcccagcctcgctgcgcagccagagacctgctatggccacgtctatactcggggtaagtcgcaagcgcggcaacgcatttgcttgttttaaccggagtaatttttcgttatggcttctggggtctgcggctccggagaaactgttgctgctagactgcagcttcagcttctattagcactttccacatttctgggactgatttttccccaagtttttggaacccagaaagatgcctttgcaagaaaaggccttcttaatcaagtcctttttcatttggtttcaaaaaaaagctagaggtttcttttcttcccctcgtgatttataccccatcccccgcattgctttggggattttgttgcaattatgcgacaatggtgtttccagaaaacagtcttaatcgttttgcagtccatctagtcacgctaataaacatttacatttccgcggctccccaattcgcccatgactttgaaatctgtctctggccgcttatcctcggattattgttttcataatcgctgtttttgtgtgtatgtaaactttaaaaccaaacgttttctattaaaaaagaaaagtcaagtcgtagccaccctgggagagctgcaagtttcgcccgtccctggcagctgtttcggccgcctccgccctcacccggtgtttgttgtctctggtctctgtgtttaccttcgcgcttcaccccgaagggcgacttggggtcttctgggagatgagccggtttacggcgacaacccaagtccggttgtaggcagggttgcagcggggaacacggaggcatctttgatttatagcctcagcgaagtgcaactttccggcgcctggaccttcgcccgggtgatcgcccctgctccgcgcccggtgcggcgactcctccgcgggcgcgtggggaggaggcgcgaggcggggagccaggaccccccgggagcagccgcaggggacgaggctgctcacgggtgcactttgtttcttcccccacttcctcttgccctcctccttcttgctccctcccccatcccacccactctaggaagagccgcgcttcggaacgaccccgttggccatgctggcggcgacctgcaacaagatcggcaacacgagcccgctgacgacgctgccagagtcgagcgccttcgccaaaggcggctttcacccctggaagcgctcctcgtccagctgcaacctcggctccagcctctcgggcttcgcggtggccaccgggggccgtggctcgggcggcctggcgggcggctcgggcgccgccaacagcgccttctgcctggcctccacgtcgcccacgtcgtccgccttcagcagcgactacggcggcctcttctccaactcggcggctgccgcggcggcagcggccggggtgtccccgcaggaggcgggtggccagtcggccttcatttccaaggtgcacacgacggcagccgacgggctgtacccgcgcgtgggcatggcgcacccgtacgagtcctggtacaagtcgggcttccattcgacgctggcggccggcgaggtgaccaacggcgcggcgtcgtcgtggtgggacgtgcacagcagcccgggctcgtggctggaagtgcagaaccccgctggggggctccagagctcgctgcactcgggcgccccccaggcctcgctgcactcgcagctgggcacctacaaccccgacttcagctcgctcacgcactccgccttcagctccacgggcctcggctcctccgccgccgccgcctcccacctgctctccaccagccagcacctgctggcccaggacggcttcaagccggtgttgccctcctattcggactccagcgccgccgtggcagccgccgccgccagcgccatgatatcgggcgccgcggctgccgccgccggggggagctcggcacgctctgcccgccgctactcgggccgcgccacctgcgactgccccaactgccaggaggcggagcggctgggcccggccggggcgagcctgcggcgcaagggcctgcacagctgccacattccgggctgcggcaaggtgtacgggaagacgtcgcacctgaaggcgcacctgcgctggcacacgggcgagcggcccttcgtgtgcaactggctcttctgcggcaagcgcttcacgcgctcggacgagctgcagcggcatctgcggactcacacgggcgagaagcgcttcgcctgtccggtgtgcaacaagcgcttcatgcgcagcgaccacctgagcaaacacattaagacgcacaacgggggcggcgggggcaaaaagggcagcgacagtgacacggacgccagcaacctggagacgccccgttccgaatcccccgacctcatcctgcatgactccggcgtcagtgccgcccgggcggcggcagcggcggcggcggcagcggcggcggcggcggcggcggcctccgcgggaggcaaggaagcagcgtctggccccaacgactcttagaggccgggcgagaggcgcgagcacacaagcgagtagagacaccgagaacgaacgagaggttcggagggcgagcgagcgggaggcgggagggcaggggcttcagtgacgcccccagggcccgggctgggcgcgaggtggagccgctcagggctcccgggctgcggttcgcccgctgtgcgaggagctcccctctgccttccgcgcccggataagaatcgaacgcgtggtccggaaacaaaagcgaaccatcctccgacacaaacactttaaaaactgtactcccagacgtacacatacaccggagacctacaaccacaagcacgcacactcgcgcgcgcacacacataccactcgcccaaacttctcgagcg

SEQ ID NO: 167 - DNA, 4359 bp, Homo sapiens
  source 1..4359: mol_type = genomic DNA
cgaaaaggaggcggcgcaacggccaccccttccagcacctcggctttgtccttcccggggaaggcggccacatccctacccgccttgctcctgaacgtagtaaacaatctcacaaacaaccaccgctgcccacgctctccatccgtcctcccggccttatcagacctccgttctcccgcactcttcgggcagggtccctaataagctcaggctgaaagaacgtttgccacctcccccaccctcgttgaaagaaaaggaagaaaaacagcagcagcgagaaacctccggggcgactcctcccccgcccccaagcaccagcgcacagcatccccctctgtctttgttgtggttctccgttgcttcgggccacgccgttcagccaagcaacccggacctgagagtgcacagccaggactagcttagggggcgaggggttggtctttgggaaaccaagcgctcaggacagaggtggaaagtgggtcccgggagccagaaaagagagagaagggcagacggctgggtggcaaatacaaaaatagaaataatttagggggatgcccgccaggcttttgcgcctgctccttctcccccaattcggagcaggttcccttcggcctcccgcgccccggggcgccccctggcggcagcggcagcagcgggcaacgcgcggagggctcagggggcgcacaggggactcccgggcacactcagagaggcgggcgcggccccctggcggtggcgacgtagttatctggtgagcggagcctcgtccctctggtccggcgggctcacggccgtcttactaagcaccgcggccgagtagggcaggaagccgctctcggaacgcggcgccgccgcgctgcagccgaagtccgagcctcccccggccccggcgcccccgccgccgccgccaccaccaccaccgccgccgccaccgccaccccgggaacccagggctgcggcagctgctgccgcggctgccgccgccgactgactgctgtggcaactgaggcacgagcagggtgcagagccgccgctggggggcgcgccggccgccgccgccgaggaggccgcagccgctgcggctgccgcggctgccgcggcagaggccgcgctgttgagccccgcggcggccgcgggagcctggtagagaccagggtggcggaagctacacagcagctccggccgagagtaggggtgcgagagggcgcggaaggtgtccagtggccggatggaagtagcgaagggcgacgaagccgcggccgccgcgcctgaggctgcagccgcggccgccgccgccgtgacgcccacgtgcgggtagtagtgcagcggcacgtgcgagtggaaggggtagggcaggcttccggtggcggccgcgtgcgtcatcatgtaggtgtagaagctggggtcggctgggtgcggccaggacatggccaggcgctgccgcttgtccttcatgcgccggttctggaaccacacctgcggggagagacgcgccgcagcctgggttagggagcgccccgtgttcccagctcctgtcccaggacctctgccccttccggacctctgaatggcttggtctacttctctccgaccaagcccaaccccgagtaccctgtggtctcccagctgggaaagtgtggacggcagtgtgtggaccgccgtgggcacaccgtcctcaacgaagagggtcctctcccccgcgtccggctgctgctgctcctcaggcttttattcccttacttcttgctgcacttttttgtcccaatccaacctttcctctccctcccgccacccaccagtgccggtctcgctgagcacccgtctctcaatcccaggatttgtacggggattctgggcagcctttgaagagaggctgccgctcagcttttctgagaggtcgccgcgcgaagtcttgagccctccgaactgcaaggacctgcccctaggggcacgggtccgattttgatattgaagggatgattttgttggaatcgtttgccttaaatgagtgggtagagcaatgtctccataaacggggaaggggacgtttccacccctcccaacactatctaataaagacatcattcgtcacaactctaaattaaagaaagcccgatcaaaccagagggagacttccacactcctcccctaccccgtggagattttttcttttttctgcagtgtcgaacgctccatgaagggctcaccaaatcgcctaacctcccggctatctctcccagacgtataataaaattaataacctaaagttatatataaataaggacaatttcgttgcatttttccccgcaggaggttgcccttttttcgttgcccaagaggaaaatgttcaggaaactactgtctcaaaccaatcgattttaaagatacagtatccttttctccgtgtaacgatttatgcggaaaataaatctccaagctcaagagcaaatgaaaagtttcacctctggttcctgcttgaggaacaaagaccaactgggcttgccgcctaggggaaagtggggccgtgggtatgggcgagggggcatctggccaggcgttgggcacaatggagcaggggcgagtgctttcagcattggagtcaccattcgggggccttcttagatccgtcaggccggacaaccgttcggattcggtggccgggaaataaataagccaattcctttggtgactaccccccgcggatttccagacccttagctaaatctagccacccagaaaggggaaaggggaaaaagaaacaatcaacccagatgcccccggggaggccagagcaggcatgcactggaattgataccttgatggtggtttcgggcaggttgagtgccgcggccagctcgcaccggcggggccgcgacacatagttctcccggtagaactccttctccaggcgcgcgatctgctcgcgggtgaacgccgtacggtagcgccgcacttgatccgcgccagagccggagccacccagcgccgcgctcccgccgctgcctccgctgcctccatgcaggcttccgaggcctgagcccgacgccgacgtcgtggtgccggcagccgagccgctctctgcgtaccctggcaaacaaacgaccaacagcgcatgagtggctgtaggaccaacagcccggcgctggcgctgcgcgcggatcggggaagccccgtcaggaaggagagtcgctgccggaattgatggggtctgtcatgcttacaaattgctgccgttaatggaatcaataaagtttggggagcccttcataagcaaataatagaaacggaattaggagatttcttttttaataattagaaattttcaaccaaggaggaaaagtggccgagggaaaatgcctacctctgggcgcagtttgggaagctctgggtttcccatggtctggagaccgcagggcagctttttatacggcctctaacctttatctgagtcttcggggtttcaaatattttaagagttccaacacagcagtagctcaaacccaagccaatagggggtgaaatatacttttcaactctttctcccgctgacctaaacagagacgccggcgaggcttcctccgacttacccagaagaatgccccctccagccctgagggcacaggggcaggggagttcagagtagttgcccagggtacctttcccaaaagcaactcggctgctgactaggggtctaccggctcgctcgggctcttagacacgagcgcagaaacattttctcggactcaggagcgagggcgggtgggcctggtgttccaggctccgcaggcctgagcctggcgggaaagctcaaggagcagaactggaaggcacggtcccagacatgcgtcccgcccccgcccgtgctagctcggtgtagcttgcctgtggagggtctgagaggggaaaaggcaccgggaaaggctggcgggggccgcggaggagcaaagaggatgggactggagagcgcggtgcggccggcgcggttacctttgccattgttttccttaagctgagcggcgccgaggcccccgggggagcgaagcgcggagcagcccacctccacgtcgctgctcatgtcggcctcagcggccgcctctgaataatggcccggcttcttgcggctctcggcggcggaggagatttcggaggagacggtgctttcgctgcccgtgtgctgcaggttgaacaaagtgtctatttcgaatttgcccttggcggggagttctcccagagcgctgtgcaggggggcagacggcaggcgcgggcttaggcgagccgggtgctgcgaattttccagggcctcgagcacagcattgccagccgagttggacaaattggagaatctcttgcccgccg

SEQ ID NO: 168 - DNA, 304 bp, Homo sapiens
  source 1..304: mol_type = genomic DNA
cgccgctgtaagtaccttcccgcttcttccgaatcgcgaagagacggtactgatcgcggggaagtggtgggctaaggggccaagccagcggagtgggcggatagaagaaaggccctgcctcccccaccccacccccaccctccacccccggcctcgctctgcgaggttagcccgcttcgggaggtggaggcgctaaatcgaggtcccggaaagcttttctggggcctggttcacagacacctgcctgagagaacggtccgcggggtctcgcccttgtctccttgggcctccctagaccttcgcg

SEQ ID NO: 169 - DNA, 201 bp, Homo sapiens
  source 1..201: mol_type = genomic DNA
cgaatccagtcccggccggcgctttgaagcctgtgctgtgcgattttctcagcagtgaggtaggaatagacgcggctaatgacaggaaggtcgcgcgggtgagctgacctgtgtgtggcgcagccttgggttccgcaaatagggcacccacagtaacacgtgtggcgccgaccccgccgtgcgcaatcggggctttatacg

SEQ ID NO: 170 - DNA, 247 bp, Homo sapiens
  source 1..247: mol_type = genomic DNA
cgatgggaccgcggcttgacacgcattaaggagccactgaaaactccatctaagcagaaaagcggcttagtcatccatgcgttctgggcaagttatcctggccgcaccacgtggaacggagtagatagagctcgggaagctgagcgcagggcggacaacgggaaggccagagccgtgggcaagcacgcggagggcagcgaggggcactggcaccggagccgcggggaggcaggaaaacgcagaggcg

SEQ ID NO: 171 - DNA, 562 bp, Homo sapiens
  source 1..562: mol_type = genomic DNA
cgctcctgtcccaagggcgcgtctctctctcccacgcacaaactcgggtcctgtttgctcttctctcacccaaacccacagtctggcggacgcaccccgccccgcaggcagctttctctcgcacgccgggcgcccggtcccgacggtcacccgcactcccgcacaggcatccccacagcctgcgccagcccaccgctcagcacccgggaaggccgctcgccccgcgccgcgccctcacccgctgtcaagctctagctccagaagggactgggtcctctccgagttgcagtgcaggcaccacatggcggccgcagtgggagcgggccgggccgggagccgggacccaggcccagcagaggccgccgactcccgccagcgagccgccggatcccggggccgacacccggccacccggagacccgaccacccgccacgcgatcacgtcggggctcctggtccagcgtccgccggctcccgcggctccggccccaagtccgagccccaggccagccaagcccgcccctccgccgccgcccgcttggcgcagcgcgaggccgggccg

SEQ ID NO: 172 - DNA, 1014 bp, Homo sapiens
  source 1..1014: mol_type = genomic DNA
cgcggggagcgggctggaggattctgcaagcacaggcccctatggagcgcccgttcgggggacgggatttccctccctttcccctcccccgcccgttcggaattccgcccccgccgcgggcccggaacaatgcagccgcccgtcccggggaccagccacttaccacccaggtcagtgccccgttgccgccgcccacgccggctcccgcgccgcagccacccgacttggccatggcgagtgcctccagctcacaggccctgaggccgcagccgccgctcccgcctccctgcgggccgctgggccccgccgctccgcacccaccgctccactcgccgcactcctagccgcgccgacccccgcgccccatcgcgaagatccggagcggacgtccagccgagcccgctgaggaggccgcgccgccggctgccctcaaactcgaggcgcggcgtccgcgtcgcccgggcctagcgcggcggctgaggagaaagcagggagcgaccggcggcggccgagcggcggcgttgccttctcgcccgcccgcgggcgccgctgcaggccgggctgaagcccgggcgtgcgagccgcgagggcggccggggagcccgaggcgctcggacgtggcgaggacgcagaggtgccttgtccttctcacactccgcgaaggccagccactcgagtcgccagagtagtcgtcccggtcgccgccgctgcttcaaaggcagccttagcctcgctgcagccccgatttcctcacacacacacaccgagagggacaataaacagagccgccgccgccgccgccacggtcacctccctcttgtccggcataacaccgcacacacacatttgcacaccggggagagagggagggcgcggcggccgcccccgctgtcagtcagcgccatgctgtccaatccgggccttgtcgccacagcctctcggccaatcccaaaattccaagccccctggaggggcgggacccgaggggggaaacgtcattgcgggtagcaaccg

SEQ ID NO: 173 - DNA, 235 bp, Homo sapiens
  source 1..235: mol_type = genomic DNA
gaccggcgcttcgctaacagcagcgaccgcaagaagcacatgcacgtgcacacgagcgacaagccctatctttgcaagatgtgcgacaagtcctacacgcatcccagttcgctgcgcaaacacatgaaggtaatcgccgcactctcgtcgccccctttgaggcaggagctctcttggctctcggcttggggtcgggcggcgagtggcagacaggcggtggcgggagccagaggaa

SEQ ID NO: 174 - DNA, 559 bp, Homo sapiens
  source 1..559: mol_type = genomic DNA
cgggcaaacctccggaggccccggtgcaccgcgcgtccagccggcccaactcgagctagaagccccaaccactgcccagtgcctgagttgcagtcttgggtcctttagaaacctggagatgtgcgtaaaattcagatgccggtattcccgaacttccccaggcctcagcatatctcggcggcctgtggacagatgggaggctaccaatcgctccggcgtccgcagcccgacccctgccgccagaccccggacgtcttccggataataaagttcccgctctaattcattttccctaatctggacgcccctaatctacagcttttattgcgcccagttaaaagtcgagggaattcgctgtccctccgcgctcggataattacccctaaatggccacggcagccccttgtgtttcctggagattagaaccccgcagtcatcaatggcagggccgagtgagccgccaatcacctccgctcactccctgagagccgctggcctgggccgcaggaggagaggccataaagcgacaggcgcagaaaatggccaagccccgaccccg

SEQ ID NO: 175 - DNA, 3638 bp, Homo sapiens
  source 1..3638: mol_type = genomic DNA
cgagcgcggcggctggctgacgctccctcccgcctccttatcccgggccctcctccctcgctctctcctagtgtgtagtttcggcggctcggctgctactgccgccgccgctgcagcagcagcgtcagggacaagcctgatccgtgagcgagcgagcacccagcagcaaccaccactttgggcaacttgcgggtttcctgctcgcgggtagcgaacgggagattcccactagcggttcagtgtctccgagccgctgcagcccgaggggcggcgggagcgcccgcaccagactctcccagacgcgaaaagggacgagacaactgcggaattcaccagccgtgccagggcacttccgaggccacaaccgactgacactttttctccccttggcaaactggatttttttttttaagctacttggcagctgtctctgactccacctccccctccacccccgcccgaaagcctttggcttttcttcggaaatccagacagaattgggcatctttgttaattgccgttggggacgcccggccgtgcgctttcgccggctaacgtcgcctgtgctccgagcctggtttgctcacctttgaactgcaaagggatcaagttcagcttgagttccctgcattgggaaggagagagagcgtgcaagagagcgagtgggagagggggaaaggggaaaggagaggagggaggagagagaggagggaggagagagaaagggagggagggaggggagggatcgagagagagcggggagagagaggctgcaatctcctccctgaatcgcgcacagcgctgcagatcccactgctccgacatgcgggccgaatgcaggtgagaaaaggcacggactctgcggctgcgaacccaaacttgggcaccgcacggtgcgcactgctcagccttcgcccccgtgggcgaaaggctgctgcggtttcaggcggctgcttcgtgactaatgaccttgcgcagagttgttaagaaaaaagagaaacccgcgctctccggggtgagaagggactgactctgggcgtctctgaagatggctcgggcttctctttggcgcgccggggggaccctgacactgaccgctctgtgacgcgagtagtctcccctgcaccgtgcccgaagcgacgtgccgggggatttttcattctcgatctgttgactggctcccccgctgcatgagcagagtcggagttgagactggcttgttgctggccccagcgcctggtgcaggaagcgactcacgtttgtctgggtggccggagccggagcagagcctgggtttggagtgagtgcctggaacgtgaattggactcaactcgagtagcagcaaagaccagcgggctggcaggcgggggaggctgcaggctcattccccacctcttcccagccccactgcccgtctgccggagcggttctggccccttccgacagagcggggactagagccggggattctccgcccgctgaggggatgactctgggttgggggagcgccgaacccgcggcgcgcagtgtcccgtgaactgtgagtactgcgactgaacggcggcaggcgagcgggcgattagcacccattgcatgaattatgaaacaataactttcggaagaagcaggaggaaaaaaagaagcatctatcgctgccctcccacccccattcccggccaactctccacgccgcttttgccccctccctcccctccctctcgctccttcctttccgggagaggggagaggactcgggggagggcaggcggccggccccggaggaggggggcgccgagggggctgtggttagaaggagcagtagcagcagcagcaggagaagatgctgaggatgcggaccgcgggatgggcgcgcggctggtgcttgggctgctgcctcctcctgccgctctcgctcagcctggcggccgccaagcagctcctccggtaccggctggccgaggagggccccgccgacgtccgcatcggcaacgtggcttcagacctgggcatcgtgaccggatcgggtgaggtgactttcagcctggagtccggttccgagtacctgaagatcgacaacctcactggcgagctgagcacgagcgagcggcgcatcgaccgcgagaagctgccccagtgtcagatgatcttcgacgagaacgagtgcttcctggacttcgaggtgtcggtgatcgggccctcgcagagctgggtggacctgtttgagggtcaggtcatcgtgcttgacatcaacgacaacacgcccaccttcccgtcgcccgtgctcacgctcacggtggaggagaatcggccggtgggcacactttacctgctgcccacagccaccgaccgcgacttcggccgcaacggcatcgagcgctacgagctgctccaggagcccggaggcggcggcagcggcggcgagagccggcgcgccggggcggccgacagcgccccctaccccgggggcggcgggaacggcgcgagcggcggcggctcgggaggctccaagcggcggctggacgcatcagagggcggcggcggcaccaaccccggcggccgcagcagcgtgttcgagctgcaggtggcggacaccccggacggcgagaagcagccgcagctgatcgtgaagggggcgctggaccgcgagcagcgcgactcctacgagctgaccctgcgagtgcgcgacggcggcgacccgcctcgctcctcgcaggccatcctacgggtcctcatcaccgacgtgaacgacaacagcccccgcttcgagaagagcgtgtacgaggccgacttggctgagaacagcgccccggggacccccatcctgcaactgcgcgcagccgacttggacgtgggggtcaacgggcagatcgaatacgtgttcggggcggccaccgagtcggtgaggcggctgctgcgccttgacgagacgtccggctggctcagcgtcctgcaccggatcgaccgcgaggaggtgaaccagctgcgcttcacggtcatggcccgcgaccgcgggcagccccccaagaccgacaaggccaccgtggtccttaacatcaaagacgagaacgacaacgtgccgtccattgaaatccgcaagattgggcgcatccccctcaaggacggggtggccaacgtggccgaggacgttctggtcgacacccccatcgctctggtgcaggtgtccgaccgagaccaaggcgagaacggggtggtcacctgcaccgtggtgggcgacgtgcccttccagctcaagccagccagcgacaccgagggcgaccagaacaagaaaaagtacttcttgcacacctcgacccctctggactatgaggccacccgggagttcaacgtggtcatcgtggcggtggactcaggcagccccagcctctcgagcaacaactccctgattgtcaaggtgggagacaccaacgacaacccgcccatgttcggccagtcggtggtggaggtttacttccctgagaacaacatcccgggcgagagggtggccacggtgctggcgacagacgcagacagcggtaagaacgccgagatcgcctactcgctggactcctctgtgatggggatctttgccatcgatcccgattctggggacatcctggtcaataccgtgctggaccgcg

SEQ ID NO: 176 - DNA, 261 bp, Homo sapiens
  source 1..261: mol_type = genomic DNA
cgtcaccagttactcccgtttagccaacgagctgcgtgtgagctgcatggagcggaaaaaggtccaaattcggagcttggatccctcctctttggcgagcgaccgatttaacttcattctggcgagtaccaacagcgaccagctcttcgtagtgaaccaggtcgaagtcgaaggctccaagtacggcatcatcagcctgcgaactctgaagatcccttcgttccacgtgtacgtgctcagaaacctctacgtccccaaccg

SEQ ID NO: 177 - DNA, 3140 bp, Homo sapiens
  source 1..3140: mol_type = genomic DNA
cgcaacttcccgcagttactgccgctcagccgcactaaggaggtccggagacttggaagaaacttcggagactcgctgaaaggcgaagaactgagcgagaaaaggaccccaggctgggccctgaagtccacctggaaggctgggagcctccgcggcgctgcggcctactaccccgaagactagcgtgatcttggggcaactcagtttgcctcaagcaaggtctttgcgggatggtacattttccccaggccgtgtcccttcatctcactcttctcctggctcgctcttgtgttagggtgttgagctccacgatgtccccaaatgtgcccggacgcagcgtgtgccagcccccagagtggaggcaggtgggaaagcgcgggcccagcgctggtgagcggctggaagcgctcgccctgccctggggacgctggactgtcttcaatgggctcacccaggccgagtgtactcgggcggcgagggcgaagaggtgaggtggggacaccccaggcggagacgtgcatgcaaatacactcacttcccgggcggtggagggctcaccgctgccgggcctcactgcttcgaagagaggcagcgggttaggctggcgaaggcggaatggggcgctggacgacagggaggagccgggaaagcagagatgcagtggcctctcctcccctctcctccccatctttctaatccaggccaggcctgcggtcctggccaggccgtttcaggaacagccagggcctcggacgagacacacgtccctcaccgcgttgtggtccacacgaacacacacacaccggctcagcccaggcgtactcaagacttagagagagggaggcgcttggatacagccacgcgagctcagaaaagcgagaatccctttctggaagccctggcccaacctaactggtgtgattccggcgctgtcaaactactggggcgggccacaggatggactgagcggcatgcacaccaggggccgcgacccgggagtgggcagaacaggcacggcaggcaggcatcggggcgcgggtggtagtactcacgaggggtacaggccaggcgtgcgtccctccagggcgggctctgcatcactccgggccagaagatgggcgtccggccaggcagctcagccagcggcttggggtaccggcccacggcggccacggccgcggcgctggggctgaagtagagcccgggcggcggcggcggcgggctcaggctgctaaagcgtggcagtccggccagcagccccgccggggatgaggcggcggctgcggccgcggcagcagccgcggcggcggcagaggcggaggaggcagaggcggacgaggaagaggaggaggaggaaccggagggcgaggcggagggcagggcggcccccgaggccacgggcatggagggccggctcaggatatcgttgatgccgtgtggggtggcggccgagagctgctgcggggggctgccgagggatgagagcccccccgtggccgggggcttcaggccgcctgggttgtgggtgcccagaggcggggagggcgacgaggaggacgacgacgaggacgaggaggagggggggccggcaggcagcgggggatacgcggcagggtacagcggggtcttcatctcggccatgctgtgcagggcggccaggggagggctgctgagcaggaatgcgctctgccgggtgccctccattgcccccaccgctaacatcccacggccacgccggagaccgtagccttgcagcgagggcgctggctggtgccccccgcggggctcagaggagccggaagcgccgagggcgcgagcggagaggcactcggcgcgcccggaggcgagctgccaactgaaccaaaaatgccgctgccgggagttgctcgcctagctgcgcagcagagatgtccaaaccctccacgcgggaggcggcagctcgccgagaaaagcaggcgtcccggcgggctaggcagtcctttcgttccgcgagtcctagattcgatccctggctattctctcttcctcacttttcctagctgttcaaagtgcgctacttctcatcttctgcccccgggaaataagcaaaacaaaacccaggctggctccggagagtttgtagcaaagttagttgccgaatctccactttgaagttggagggtgggggtggcttgctttctttggggaggttcaaaggacgccttgtgcagcccgtggcgctcctctgatcttactgggatgctctgctctttcggtcgcgcggctgattcgcattcgacgctcactgtgcccggggagaaagcgcatccagcccgcgggagatctagcctctgtgcgggcttcctccgcccacgctgccccgggctgctgcgccagaaagggcagtgccacggatccccgcggctcccaggtcctcacctccacccgccttgccctctggcccacggggcactggagttgcaatattgaaaagaaaaagggggaggaaaaacacagaaaaacaaaagactaagtgtgaaaagtctgacggctgggtttcggcgccgctcgtcagtccacttctgcaaacgggcccggcgacccccgccccacccccgctccctctctccctctcactctcagcctttgactctcctctctggcattttcttcgcggcttcccaggcttcggtcttctaagtcctgtcctctggccaaactgaccctctccgcggctcctccgctctttctctcctttcctctctccctccgggcctgacagttcagatgaagctctcaaaggtaataaaacatttgcatcactccctacccctctttagctgggggacgaaagggagggggaagtgggggaccaaaatgagaactttgagggacgtcactccgaggctgcgggggccggcgagccgggcgcaggccgggggcgtaaccaccgtgtccaatagctcgccgcctcgatccttgcgtcgcccctggggctcgcccactcgggggcgccgcaccctctgattggctgaggcgggccaggcgtagggccgcgcccgcctagtcccctcccgctccctacttctttctcctgtgggcggggccaggaggggcgggagctggtgagtggtgcggttccgcgctggggccagtggccg

SEQ ID NO: 178 - DNA, 524 bp, Homo sapiens
  source 1..524: mol_type = genomic DNA
cgcggtgcggagcgtctggagcggagcacgcgctgtcagctggtgagcgcactctcctttcaggcagctccccggggagctgtgcggccacatttaacaccatcatcacccctccccggcctcctcaacctcggcctcctcctcgtcgacagccttccttggccccccaccagcagagctcacagtagcgagcgtctctcgccgtctcccgcactcggccggggcccctctcctcccccagctgcgcagcgggagccgccactgcccactgcacctcccagcaaccagcccagcacgcaaagaagctgcgcaaagttaaagccaagcaatgccaaggggaggggaagctggaggcggcagaggcggcggcgggagagctgtggggttgttcaggaaaagttgggctgggggctgaggcacctgaagcagtgaaccaggactaggtaggaagcaagagcatcctcctgcgcaagcggagacgcaaacgcgctcctgaacccatgcaaccgcccggcccagcgccg

SEQ ID NO: 179 - DNA, 1966 bp, Homo sapiens
  source 1..1966: mol_type = genomic DNA
cggagccagacctgacggtggaaatctctgagagcgccacgccaggcactcgcttccccttggagagcgcattcgacccagacgtgggcaccaactccttgcgcgactacgagatcacccccaacagctacttctccctggacgtgcagacccagggggatggcaaccgattcgctgagctggtgctggagaagccactggaccgagagcagcaagcggtgcaccgctacgtgctgaccgcggtggacggaggaggtgggggaggagtaggagaaggagggggaggtggcgggggagcaggcctgcccccccagcagcagcgcaccggcacggccctactcaccatccgagtgctggactccaatgacaatgtgcccgctttcgaccaacccgtctacactgtgtccctaccagagaactctcccccaggcactctcgtgatccagctcaacgccaccgacccggacgagggccagaacggtgaggtcgtgtactccttcagcagccacatttcgccccgggcgcgggagcttttcggactctcgccgcgcactggcagactggaggtaagcggcgagttggactatgaagagagcccagtgtaccaagtgtacgtgcaagccaaggacctgggccccaacgccgtgcctgcgcactgcaaggtgctagtgcgagtactggatgctaatgacaacgcgccagagatcagcttcagcaccgtgaaggaagcggtgagtgagggcgcggcgcccggcactgtggtggcccttttcagcgtgactgaccgcgactcagaggagaatgggcaggtgcagtgcgagctactgggagacgtgcctttccgcctcaagtcttcctttaagaattactacaccatcgttaccgaagcccccctggaccgagaggcgggggactcctacaccctgactgtagtggctcgggaccggggcgagcctgcgctctccaccagtaagtcgatccaggtacaagtgtcggatgtgaacgacaacgcgccgcgtttcagccagccggtctacgacgtgtatgtgactgaaaacaacgtgcctggcgcctacatctacgcggtgagcgccaccgaccgggatgagggcgccaacgcccagcttgcctactctatcctcgagtgccagatccagggcatgagcgtcttcacctacgtttctatcaactctgagaacggctacttgtacgccctgcgctccttcgactatgagcagctgaaggacttcagttttcaggtggaagcccgggacgctggcagcccccaggcgctggctggtaacgccactgtcaacatcctcatagtggatcaaaatgacaacgcccctgccatcgtggcgcctctaccagggcgcaacgggactccagcgcgtgaggtgctgccccgctcggcggagccgggttacctgctcacccgcgtggccgccgtggacgcggacgacggcgagaacgcccggctcacttacagcatcgtgcgtggcaacgaaatgaacctctttcgcatggactggcgcaccggggagctgcgcacagcacgccgagtcccggccaagcgcgacccccagcggccttatgagctggtgatcgaggtgcgcgaccatgggcagccgcccctttcctccaccgccaccctggtggttcagctggtggatggcgccgtggagccccagggcgggggcgggagcggaggcggagggtcaggagagcaccagcgccccagtcgctctggcggcggggaaacctcgctagacctcaccctcatcctcatcatcgcgttgggctcggtgtccttcatcttcctgctggccatgatcgtgctggccgtgcgttgccaaaaagagaagaagctcaacatctatacttgtctggccagcgattgctgcctctgctgctgctgctgcggtggcggaggttcgacctgctgtggccgccaagcccgggcgcg

SEQ ID NO: 180 - DNA, 3844 bp, Homo sapiens
  source 1..3844: mol_type = genomic DNA
cggattacgcagccacttctccctgcaaagtgcacgctcccgcgccgcggccgcacgccgagcacggcaagcggtgccccgccgcgcgcccctacacacctgcctcggccggggtccgtctctcgccgtctacctgttgcagcgcttgccaccgccacgaaacccctgctgcggagcccccctaaatccaaccacatccaggccagcgagtgccgctctccctgcagcgggagagcccgaggcagcaacgctcttgcacccaggtgactgactgcacgggcttcctctgagcagcctgaagttggctcccgtctcccgggcccaatgacaccccgggcgcggaggggctgagaggagggggcaggggcaggggcagggccagggcgggcaaaaaagggagggagagagggcgggcgaaagcccggagggggagtgggccagctggagggctggcccttttaaaagcgggtcacatgtccagataaagttcaggttgctatcccgcccccctgaacccttcctattggcccgaagagaagcaaggctccacgccgcacgggtcaggcgggcttcttattggacaatccaggctgtcagtcaccaggcgggcgacatgactgatacaaagttgctgcgacacagcctggactcggcagcttccttaaagccgcagccgcggcgggagcttggggtgccgggcggtggctacatcaggcggcctccgcacgctgcccccacccccatcgctcggccgccccaaacttgcttacgtccaccctgctctccttctgaccgaccccaggatatctggagagatgtgaaaagtgtgctgacgcggaagaagtcggtttttccgcgagcaaagtgtcgtcccctcacgctctccacctgcagctccaggtgagctgcagcccagaccctggcgccgcccaacagcgtccgcgcgcggggaggactctgggtgggcttcacccgaccctcgaggttcctctcccagcccctttccactgtctccagagttacctctcagcagagcagcagcagccttcgctgccagcgagcaagacgatcacgttcactcgatttgaatttagttgttttaaccgtgctggaaaaaccctttaaacacagctttcctcccatcccttagacccctaacaccaaaaaaaaaaaaaaaaaaaaaaaaaaaaaaaagagagagagagaaagataaatgaaagaagtggcagggtcaagagctgctcctctacttccttataattttgtcactgtacttactactttagtattggatttttccactagttcaatttgaaagcaagcttaccatcgaggatattcacgttataatgggtggagggggtgggtcacataagtgggagaaacagacaggaggggggaaggaaccaaactgttttctaaggatcggttgtctgcatgaagaaatttcggtttccctccaaccgccccctcctcgattctcctcccgcccacccaaatgctgcagttactaagcaaaaggatggggttcctaactttgatttctaaggacttttaggcttctcatgtaaggccccgttttaggctccccgctggcccggagtcagcatcagtgacgtatacacctaaatctggtcccccggccctcggcgacccgcagcccggcggcggcagctgtgcggcctcccgcctcaccctcgcttcgcgcccgccaaagcccgaggcgggggaccgggggacaccctgtgcccaaaggcccctcctctccttaacagttagttaagctccgggattccggacccctaacccagggctgagacttgaactctcagccgctccagtcaggaactccctggagataggggcgacagcggcagcgccgcgagctggtcctgaccccagactctaatgcccctcggtccccagcttggagctgcccccggcgtccccgcagccaggagtccccgccgagcgtcccaactttcccgcggggcccaccgtcacgcgcactctcccggcccagcgaactgggcatcgatctcacgtcggcagagcgcgggcggcgggcgggagcaaggggagtcccggacctagagcctgggcacgcgaggcggcggcggctccgcaagcgttgccgaattattcattattaaaggaatggacgtgtctcttttactaagtttttatctcctttgaggagggccggtcttgccctggatctcagcttcttgcaaaatctaggttagaaccgtgcaggggcagccgccgcgatcactcgcccgccacccagcacccttgccccagggccgccagcgggcaactcgtctcctgcccgcatcccacctccgctcgccaacccgcgccctctgccctgggcgcgcaacctgccttgcccggggccgcccctgcgctctccggaccccctctcgccgctgtcagcgcaaagtgactgtcgctgcactcaccttttctaggacatggtggggaggctggggcgggggaaggggaacggctagactcccgccgccgctgctgccgccgccaccagcaagtccaatattggctgatcggcggtgaggatggagaggatgataatcccggcagtggcagtcgccctgctgacggtgggtagagcaatagtgctgttaccctacaagaggcggcgaggcagcggcgccaaatcgcgcagagggggacgcgggcacccgcccgcccccagcggcggcggccgggcctctggcgggcccctctcccggggctgcgcgtccgctcggcgttgacagcggcgtccctgggagccgggaagtccggcaggaggatcccgcgcccgccgctccgcagcgccactctccgcgccccctccccgggccctgggtggccggtgacagctccggcggccgagcgcgtgtgtgagggggcggtcgcagggggagcctgcgcccccctccccgggtcacgccctcacgagcgggaggaggcggcaccgcgtccggccacccgcgggtgggaaacgggcaggggcgccggcagccgccctcccactacggccactgacaacctggaggccaaaggaaaactgagcccaaatgcacgcacccccttcagccctgcccgcagccctccaaagtccggccctgacctcgcgcccggcttcaggtgcagttcccacctgcggggggaggcgagggctggggacctcgcgcggggggcgggagggaaaagttttcttttcctgctcctcggctgcccaccccgccaggctgctcctctccccgcccctctcgggctcgccgcgcctctgccccacccccgccgtgggggggcgccgaggagggagcgggcgtctgtgcgcggaggcgtgcggggagctggctggggcgctgcggtcgcgaccggtcccctcgctctgacggcggctcagacgcccgcggaggcggctcggcggggcaagccgaggaaggggccgagagccgggacccctcctcctccgctcgcggcgcgccgcgcgcccccgcgtctcctggcgcgcccccgcgccctcgtgcccgctcgggcagacacctacgcagacgcgaaagtgaaaggggggcgactgggcccggcaggcgggcggccggcggcgcgcgggcacgcggcaggggacagaggatggatgggtgaacccggatcccgggattctcccgcgcccgctgcccctctgcggagcctccccggagtcccacgcccccaccccggccacggagatcaccacgtcaggagatatgtatgcgtgtgcatgtgtgtgcgcgcgtgttcacgcgcgtgtgcgaggtacgcttctgccccagactcttcccccgcaccg

SEQ ID NO: 181 - DNA, 623 bp, Homo sapiens
  source 1..623: mol_type = genomic DNA
cgagaagctgaacgagctggccgaccaggacttccccttgcacccgcgggagaacgaccagctggatttcatcgtggaaaccgaggggctgaagaagtccatccacaacctcgggacgatcttaaccaccaacgccgttgcctcagagacagtggccacgggcgaggggctgcggcagaccatcatcgggcagcccatgtccgtcaccatcaccaccaaggacaaagacggtgagctgtgcaaaaccggcaacgcctacctcaccgccgaactgagcacccccgacgggagcgtggcagacggggagatcctggacaacaagaacggcacctatgagtttttgtacactgtccagaaggaaggggactttaccctgtctctgagactctatgaccagcacatccgaggcagcccgtttaagctgaaagtgatccgatccgctgatgtgtctcccaccacagaaggcgtgaagaggcgcgttaagtccccggggagcggccacgtcaagcagaaagctgtgaaaagacccgcaagcatgtacagcactggaaaacgaaaagagaatcccatcgaagacgatttgatctttcgagtgggtaaggagagggcttctgtgcccgacg

SEQ ID NO: 182 - DNA, 1423 bp, Homo sapiens
  source 1..1423: mol_type = genomic DNA
cggcgggtacgttggggatgcactcgcgcagttcggcgaaggcgctgttgatgctctgagtcctgcgccgctccttgcggttggcggtgcctcggcgcttcaccgggcgcggcccccccaggcccgggggcccggcgcccggcggcacccccccgtaatgggagtggtccaggccggcggcgccgctggcatactcggggctgtaggacagggccatgctgtagtcggggggcgacatctcggggtggccgatgagccagccatggaagtaggggttctcctcatggctgcagcggctggcggcggcggcggcagctgcggcggcggcggcggcaaacgggtagccctcgtggtgcaccaccgggtggtggggaaaaccacctaccagactcatttcgccctccgcgcccctccacgcgccccagcgtgcgcgcagccccgccgcgccctcggcccgggcccctgcctcagcgctcggcgtcctcccccaccccccaccccccagcccccgggcgcccgggcccgcccggcagccgcagagggggctgctgcagcccgggccccgtccccccgcctggccagccgggcccgcctcagcagcgctgcggccgccggctccccatggggcgcggcgagctggtcctggcaccgtgcgcccctggccgccgccgccgccgcctccggttcccgccttgctccacggcccgcgcttcggctcctgcttcccgggctgctgcgcggaggcagaatcctctcgtgctcatacaaaggtgccggggctcccgcgaggctggtacgcggagtctcgggaatccaagcccgggccgcggtcctggcaccgaagctaacccggaatccaggggcgagtctgagccgcggctggaggagccggtccagctgtgccggggggcggcggggtcgactgctcactgcaaagctacctccgtgcgaccctccccttcccgcctcttcccctccccccaccagcccgatctgggttcttgggcgcttattgttttaatgaaatttttggttgttgttgttttggtccttaaatgtgattttagctgcgagtaacgtgtcctcgctcctctcgcgctctctcggagggttttcagagaggtctcagtgcatccattttctcagatcctctcctttcggggcaaggatctggggccctgaatgagccttggagctcgaagaccgcggctcgggctctcggagggctctgcgcggtggccgaggaagctccggggtgacggcggtggttctcctgggctggacgacgtgaggggagcaagcggattttcccagcaagatctccatgtacagctacatctttagggccgctcgggttaatatatgtcgtcagaggctcctcacgccaatcccggggcggcaggggcggcgcctcgagctctcg

SEQ ID NO: 183 - DNA, 2549 bp, Homo sapiens
  source 1..2549: mol_type = genomic DNA
cgagattggccgcagatgccccgacgggatggcctggggagccgaaagctgtccccagccccggacagctactgcccacgccgcgcaggccagcctcacacagtagcgaatctccttcacgcgacttcctcgcaggcctgtgaacccgcaggggaggcggaggtcggttcaaaccctgcgagagcccctgcccagcgcggagtcctgcacacggtggaaagtccttacgcattagcgggcaggattaaacgacttcacaccacgaggtgcaagactgggcttcggggccttcctcggtgttgctattttaaaagaagctgctcagtaacccagaagaagatgacgggggacggtggtgtagacccggctggagaggagtccagggcctgggaggcgccccctctgactgaggggctggggaccctgaagcttggcgtccaggccccgccagcgctgcacccggcattgtcccgcctggggcaggcgtccggtccctcggcagtgcgggccagcacgtacgtagtagctgctgaataaataaatgcctatgcaacagggaacgtcccggcccaccccggccgggccagggcctaggaagggcgggcgtccggcgccgccgtgggtccctccggcgtggccgcccacacctcctggccacggcggggctgcggtggggggacatcctggggcgggcggctggacggaagggactagagtcggggcgcgtccacggccaaccgccccgaaccaagcaggggaggccagggaccaggagcttccggggccccgcggctggagcgcccggacgcgaaggggcggcgggtgacggggcgcggtgcgggcggctacagggccggacgggagttcctccgctttccgccgcgcggctcgcggtctcagccgaggtcatcgagggccaggagccctggcggaggccccgccgtcccggacccaccgacgcactcccagggcttggcgctttcccaccaccccggaccgcccttggggtgccggaggggctcggggcgcggcgtggggagcctttgtgccctctccgcttgttgaggttttttctccggaggagagctccccgcccgccacccctttcctctcgaaagttttgtggtttccctagaagacgaaagaggttgagaaggttgcggagacttttgcaatcttccccgtccagaaactctaaaacctctcggcgtttggcccaggggagctatctggggtcaaggggagggagctggccgcacattccttcgcccgccttcctcccacctggagccgcggccccgcggggcagtgcgtccgagctggtgcggcgacgccggcagcggcggggttaagctcaggaatgcggcgcgaggaatgcgcatgcagatgaggcgggcgggcgcatgctaatcgcatgcaaatgaggggcccgccgctgccgcggcagcgccgcagcttcccggatccgagcccagacggcggcggctccggcagcctgggcgccgcgaccctcggcggccagagggggacggggcggaggaggaggaagaggcggaggcagcggcggcgagggggaggagggttcgctcgccggctccgacgcagacatggtggactgatccgcagcggggccgcgaacagcgtttcctgaggcacctcccgcgcgtggttccgccgcgccccgcgccctgcgcccctcagcccctcgccggcgcccgcgtcgcgggttggcagcctagcccggcagccgcgttcccgccgcgtcccgcgcccggtacctatggaggcgccgctcgccggcgaggccgccgaccatgcctaggagggccgggagcggtcagctgccgctgccccggggctgggaggaggccagggactacgacggcaaggtcttctacattgaccacaacaccaggaggaccagctggatcgacccccgggacaggtgggcgccggccgcgggggcgcgggcccgttcggacacggcggctgttgtcccggagacccggccgcgcggggggctggggcggcgccggcccgcggtgccccgaggggtcccgggagggatgtggggctggctgctccggcggggccgaggagccgcccgtacccgggctcctccgcccaccggcttcccgacgcccctccggggacccggccccgaggcaaggctggagtcgctgccccgggccgacgccgcgcgccaggctaacggacccgaggggccacctgagccctgttaggaacttccctaggcttcgaggtgcccacgtggaggctgcaggacaccttcggcctctgggcggcgggggtgggctagggcctcactgccagttggggaacgagccctccgggccacctgtggcatccctcaggccgtccttggcgcgggagcctgggctgcgagctcttggcgggggcgcccagactggccggaggtcccgagctgggaaggggctcccgccccccgccgggccctgctagtgggtgtgactgactttgcccctcagagtttaaaaacgcgcgtttgggggtttcgctgtctgtcg

SEQ ID NO: 184 - DNA, 306 bp, Homo sapiens
  source 1..306: mol_type = genomic DNA
cgtgcgcctcagaaggggcgttaggcagaacgcgtgcccattctcaggtgtgctcggcgtgtgtgtgctcggcgtgtgtgtgctcggcgtgtgtgtgcacacgtgtgccgtgcgcctcagaaggggcgttaggcagaacgcgtgcccattctcaggtgtgctcggcgtgtgtgtgctcggcgtgtgtgtgcacacgtgtgccgtgcgcctcagaaggggcgttaggcagaacgcgtgcccattctcaggtgcgctcagcgtgtgtgtgcttgacgcgtgtgcacacacacatgtgacggtgtgccg

SEQ ID NO: 185 - DNA, 249 bp, Homo sapiens
  source 1..249: mol_type = genomic DNA
cgaccggcgtgcaccgtctcgccattcccaagggcagtgcagagcatggcacccagcacccgacaggcgaggacggcccaggacaaagcggcagctctagctgtcctcagattttgggagggggctcccaacacggacacggtaggtagttggagcaggagggacgggcaggccactgaccacgcaaccgggcctctgtcagcggccaggggcagagggaggcaggaccgcccctggacaaccgacacg

SEQ ID NO: 186 - DNA, 8587 bp, Homo sapiens
  source 1..8587: mol_type = genomic DNA
cgaggctccgtgaagagagggaagcgaggcagggggtgaagggaccgcctggccggtgtccaggatacgggtggctcaaaccaccagcagaacagcatgccgactctggcccgatggccgccttcatcccgtagccccaacccggcctaaagccgagaagacgccagagtgcgctgctgaaattcccgcggagtccggtgtgctggagagccgcagcggggtgaactcccggccccgtctctgcgtaggaggtggttcgcaaagtggccccgggagccgggactggtacccggttccccacggcaccgtccggagctctccagccacgaggcgcagaagtggcctgccagcgccttcccaggctcagggaggcgaggaggcccgtgcacttggcatcttctcccgggagccgcacggccagggccgcggcgaaacggagcccatctcaagtgcgccgcgcctggccgcgtcctgtagcccgacgaggctgaggatggagaggggagacgcagggagaagagggacgcgggacttggacccaagaggccgctccgttgccggtctggcggcggcgccgactcgggttcgcgcgttccacacaagtttcctgtctgcctctgcacacctggtggacaaaccgggcgtccaggccacaccgtcttcccccttcgcgggggcgcgggggatgtttccctccggctgccagggggctttctgggtgaagagaaagcccctccccccgcgtctcccccacccctccctccgagaacccgcggcgccgactgcgcctgcttcccccgagctggcgacttctccgcgggatttgccctcgctcaaagtttgcacaattgaaagagcccgcagagctcggccgctccccgcttccccaagggcggcgaggccggtcattggcagacgatcggttactacccagtaggggcccacgggaacccgcatctggagtcgggggtgtcacgccacgccggttcagtggctcgcggagagcgtccgggtgcacttctgccaaagatgtcccctggaggccccggccgcgcgggactcgggggagaggccgctcccccctcgctgtcaccagcgtccaggccgccggccccttccccgctgcccaaacagtagaaaagcaggcgccaagttgtttttgttaaaaaggggacacacctcggccgcgaaactgcaaacccggtgtcagacagctgtaaacccgtgtcgacaggttgtcagacagctgcgggggctggtcgggaaggagcccacggcctccgggcccacaccccgccgccccgacgcgcgcgcccaccgcgagagtagctggccgggccggcacggggcaccacgtgctcgcgggaggggcgggagcggccggcgagggcgggcgggaggcagggagggggcgggaggggagccaggggcggggcctgcgctcaaggggatgccaatcaaagcatcaacttcaaattgtgtctgaaagccccgccgccgagcggagggcggccgccgcagtcggcgcgcgattgcggatccgggcgcagccgggagccgggcgcctgcgagcaccgggcagaggagccgcgaccggcctccatctcccggcccgcccgagcgcgcccggccggccgcccgctcctccctagacccctcgcggcgccccctgcaaccccctccggccggcctccgcctccctccccgcgcctttaatactcgcccgctgcggcggtcgccgagtccgcggacatgtccttcccgcagctgggctacccgcagtacctgagcgccgcggggccgggcgcctacggcggcgagcgcccgggggtgctggccgcggccgctgcggcggctgccgccgcctcgtcgggccgaccgggggccgcggagctgggcggcggggcaggcgcggctgcagtcacctcggtgctgggcatgtacgcggcggcggggccgtacgcgggcgcgcccaactacagcgccttcctgccctacgccgcggatctcagcctcttctcgcagatggtgagtgcgcccggcctcccccgcttctcctctgtctcacccgcgccagggcaagggtggcgggtcgcccgggagggagagactacgggtggacctggtccggaagaggaactagaaaggtccgggggcaggttcccggtggccgaggccgcggcccccggggacgcaagagggctgggaggccgggcgggtgacggctgggccatctcggcctgggaaagcggaaggcccgggccagggagcgggtagcgagtgaattcagagaggccgcagaagcaggcccgtggagcggtgcccgcgctggaggtcgggggcaaactcgcctggctcggccagggcgcccgggcaggcccacggggttcctgcaggtcggcccggcgtagcgtagcaggacttcccttcctggccgcgggttccactcgcgcggcctctttagttttcgaaccgagtctggaaaacttggttttctccctctttagcagctccgagatagttgtatccgagtttgccagacagaccccttctaagcctggtagagtcaatcaaaataatcttaacaatagaggtccaaagggatggagaggtctctccacggcgtgagtgcgaatttgagattaaacaaaaattaagttgcagtaatgtgctggtgtctgaaacggtgtttgattttacttttgtaagttgcccaagttttcatttcatttgcacagaaagaaaagcacttttcttcctgcgttacataatggaggattaaagaaaacagtgtcccttggcttaaaacaaatggtgtcctcttagtctcccgtcccagtgggcgttagatgtcggggcaggcggctgcacacttaattctccgcgggggcattggcctgtctgccggtccaaatcatccattttccttggtctgactgcaaggtcggtgcttaaacttcggacggctggtgaattgtgcggcgggcgcggggccctgggaggcagccccctcctgggtcgctgcccgcgggataaagcaatttccaagcacccgcgatatctccccgctccccgcaggagaagcggggagtaaacgcccctcaagtgtgcacaagcaaagagcgggtttccctgtaacttttcttgtagttttgaaagaaagcggcccggctgcctttcaggtctcttactatcgaaaaagatcagcccccattttgttcaggcggcggggaggccgggacgcgatgagagatttacaaggtgtcctttcaaaaagaattcccagtggagacgaggctgaaacgtcttctttacaattacaaccaaaataattagaaaagcgcaaagtacattttggaacgattgggcaaaaacgaaatctagccgcagaaatgttttctctgcggcctcagtcaccaaactaattagtccaagaaatcttctggtctttacaactttctcagagtccggaactccctttgctaacattgcaactagaccattttttcagaggatgaatattttttacagaaattgcgaatgcagttgtgtgccatttgggaaccctgcctgtgtttgcgggggagggagagagcttcagtgtgaggacctgcaccctttgtggagagctggggaagggagatgtttgctgttctgagttgtttttcccacctagagggataatatgtaaaaattattcccacccaaaaggtgtgtgtttctccagctctcccactggttctgagagagtaaactcaaacccaaaccctgattctaggcctaggtttccaagccattataattgggtgtttggaagtcaaaagataaaattgtatttgaatgtctgtctgcgcaatttatggtaataatgaggcctaatgaggttgttagaaagataaaatgttatttaccaaaaaacctgatgggataatttgacttgctgtgttttactactgattataaaaagaatatcgattgcaaataaatcagcgcctctaaatgcctgcaaacagctagtgtttgctccctccagatcaaagtcaaacttaagagatgaagtaactgagaagaggcctaggatactgaaccggttcccctcctggccgccggtggctcccagcccttgcgttaatattttacaggctaagccttccttttgtattaaaaaaaaaaatggtgtttttgttattgttgtcgatgatggccgggattaaaattttaaattacctgtcacctctaaagaccttttaatgtgggtaaaccattatatgcagattaatttggaaggcaaaggactgtgctttcgttttaaattgctggcggatttagaccggtagaaaacccgggatggtttattttgattgagccccctctgggtggcagagaggaggcttgggctctgggccctttacgtttggagaaatggctttatcagctcagttgaaaggtttttccctctagctagtgaaagataaacttggaaatgcaggtttctccagcggttggtggtggggacaggggtcgcctagggaacttgcaggggccgcggcctctgttgtgctcttctggagagtgcactgtttgtggaacttttctagagtggcaaaaacgatctccactgtcggtgaaagggcagttcctgaagtcagctcatggtcctggctccccttctccccagcagtgaactgggggtgacttcctgatctgcccagcacaggagagccccgcaaagcgcctgggaggccctcgagtccattgaagcggctgcttcccactctcccgtcttggggactcatgtctctctctctctctccctttctctctccacttccctcctctctctcctcgatggatctgccctgtggcttcagggctcgcagtatgaactgaaggacaaccctggggtgcaccccgccaccttcgcagcccacacggcgccggcttattacccctacggccagttccaatacggggaccccgggcggcccaagaacgccacccgcgagagcaccagcacgctcaaggcctggctcaacgagcaccgcaagaatccctaccccaccaagggcgagaagatcatgctggccatcatcaccaagatgaccctcacgcaggtctccacctggttcgccaacgcgcgccggcgcctcaagaaggagaacaaggtgacatggggagcgcgcagcaaggaccaggaagatggagcgctcttcggcagcgacaccgagggcgacccggagaaggccgaggacgacgaggagatcgacctggaaagcatcgacattgacaagatcgacgagcacgatggcgaccagagcaacgaggatgacgaggacaaggccgaggctccgcacgcgcccgcagccccttctgctcttgcccgggaccaaggctcgccgctggcagcagccgacgttctcaagccccaggactcgcccttgggcctggcaaaggaggccccagagccgggcagcacgcgcctgctgagccccggcgctgcagcgggcggcctgcagggtgcgccgcacggcaagcccaagatctggtcgctggcggagacagccacgagccccgacggtgcgcccaaggcttcgccaccaccacccgcgggccaccccggcgcgcacgggccctccgccggggcgccgctgcaacaccccgccttcctgcctagccacggactgtacacctgccacatcggcaagttctccaactggaccaacagcgcattcctcgcacagggctccctgctcaacatgcgctccttcctgggcgttggcgctccccacgccgcgccccatggccctcaccttcctgcacctccaccaccgcagccgccggtcgctattgccccgggggcactcaatggagacaaggcctcggtccgcagcagccccacgctcccaggtacagctccaggccgcgtccacctgtcccctagctgggaatgcagaggcctggctaggtgtggtagcgtggggtgcagcatgagccgggagggtaccaggcagtggccgctgagccctggggctgcgcttaatccctgcttcaatttagaaagccagacaaggccctagggctctcccaagagagctttgccctaccggcgggcctgctacggggtggtggtggggtgaggggtgacgtttttcggcgaatctgcctgggcagccggcagaagttggtgggaaggaggcctgggacctctcccgcccgtctctccgtcctaactctgcctcttccgatctctcgcagagagagacctcgtccccaggccagattcgccggcacagcagttaaagtcgcccttccagccggtacgcgacaagtgagtgctgtttgcttttgctatgggagaaggcggtggggaggggggaggaggagtggtcgggacccgggcggagctggctgggtggcggtgggggtcgcgcagtcctagttgaaggagcgctccccgccagccctgggcgccgggcgagccgaggagactggagtttctccccagccgggagccgcgctggctgtcgaccccgcccccagggctccgctactggaaccggcgtcgcccggcgctgcgtcccccactcacagtgcccctgtcttcttgtctcgctgtgtttcccatgcagctctctggccccgcaggagggaacgccgcggatcctagcagccctcccgtccgcctgattaagggtcttcttttacttttgcgggggggaggggggaggagttggggagggagggaatgtgggaggaattaagacaaatatttcagactggtgtaaaggacaaatatgacaacgacgtcaaggactcgcatccgtcgctttctgcagaaaggggcttcttcggtcccgagctcgcgtccaggtggccaggcctctgccggcggctccagtggctgcgattatcgggttcggtaaatgcccccacgtgcttgtgtctctttccccccttttctgtatatagagtggtttcagattgtaaatagcgcgtcagcgaacttgtctaaatcatatatttttgtctaataaactaaatgaaatgacaccccctccccgctcctgctgctgtgtgcctgtccagcgtgtgtgtgagtgtgtgtttgtgtgtgaatgtgtgtgtgtgagtgtctgtgtggcagaaacagagacagagagagagaagtgggggatacagggatcctggaaccctgggtgggacccaagggtctgtggctgggggagatgggcttctcaatgggggcctttagagactgttgccacccaagacgcaggtgctttaaacatctcttcgttgtttgtggttgttgttgaatttttaaatattgtcactgtggcagtttcttgctggcagttcaattgctttcacgaacatttttctgagacataattttctcaggacataaataagttcaatttgaggcagttttacaaaacgattttataacgtcggtaaaaacagaggaaaaagaatttttattgcgaccccagaggagaacttcggattagaaaccagtttacaactagttgtctcaacggcgcatcgtggcgcctggtcgttttctgagttgagtgtgaaaataatggagtatcgctttgcatgtatttttagtgattcggttaaatcaaacacgggaagaaattggaaggctctttaaaactccacagatgggccagccgggatgcggtgcggggcttctctgcggtgtgaggtgtgaacgaggggctgaggctgtggtgggaagcgagaaagaggaggtggctttggtctcccagggaagcccctttacacttgggctccacggactgcgtcctttgccctcaggcgcgcgcaccgcgggagtccagagcaaattgcccttagatggccgcggccgggcagcggggaggcagctgggagcagcgatgttgggaaacactcgcagcggggctggcctcgggcgcgcgcgagtggggaaaggcctaggagcctggacatcgctgcggatccgggacatcagcatcagtgggttcggagcgggacgcgcgccacgcgccgcagcaggcaccttcaggaggctttgcggacccggcgcggggccttcagggcgcaggcgactcagcgttgaatgcgtgaaaactgagccagcaaacatttccaaaactgccagcgaggatgtgggctgccgggaaaaacggtctagtggggacagggccgagtcccgaagtcagagccgagtcccgaggtcagagcggccgtcctccgctcgcacccccagcctgtgacccgcccttcccggcttgctcgagacccactggcgccagtgctgcgcgtggggactccgtgcatggccgaagcgagggggaaagtcggggcgctggtgtcttttcagaggttccaggaaagagggaggctcgcgttaggactaggaggtgccagtccacggctcctacccgctcccgacgcccgcatccttctacagccctccaccccgttcctggtccctgtagaggggaaggtcctctccctgccccgaggcgggaggaaaagcggcgaagaggaggctcgaagggcgccgcgtagggcaagtgggccgaggacaccg

SEQ ID NO: 187 - DNA, 1500 bp, Homo sapiens
  source 1..1500: mol_type = genomic DNA
cgtgtactcgcgcacacgcagtcaggtgtggatgtacacccgcgcacacccaggcacatgtacacccgcgcgctcacacaccccatccagctacagcagaattctggccaggctgttgaccgcacacctgctgcctccttggccaccctgtccacacagtagcccgatcgacccccgtggcggccgagacccaggcccatccgcagccctgagacctccctagggattgcacccagcagccagtcaccggcctccgcggcctggccagttgagggtggccgtgaccgcggggccaggagcgccgccacatctcggggcaaatggcgcgggggaagagtttcctcctcagcctccccgtctccgatcgctccgcaaactccagagcgaggcacgcgcctttaaaggcaggtccgcggctctcccacgtcctggcgcccggttttccgcacccagtgtccccacagctgtgcccgggcacagaggcgcggccagaccgcactccgcgggctgcaggtgtcccggcctctggcggcgccggtgcggcccggaggtgggagcccgcggagccactgcagtagctggagtcccgccgagtccccagccccaaggcagggcaggagcgcgcaccggccggaggtccatgctgagcatcgcccgcgccggtgcccggcagcctctccaactgtgtggtccccgcgcgggcagagaggcacggactgcaggccgtgggcagctccatcttcccgcgtcctcctcctctggcgctgcccgctgtctcccgccttccctctgctccccgctcgcctccgcctcagcgccccgctgacctcgcctcctcccctctgctctttgtccctgcactctcccctcctcggtcctctgacccccccgccctcacctcctcccctcctctctcccctgcccgccccgcgctctcccaccgctcccgccgcccccgccgccgcggctgccactccgccccccgcgccgcacggagcttcagtaataaccccggcgcggcggcggagtcgctgtggggaatcctcccgcgctctgcctgggtcgggtcctccctgcccgctcgcacgctgccggccggggaccctccggtggcccctagcccctcggagcgctcctggatgaagccccgcgcgcgcggatggcggggcttggcggcgctgtggatgctgttggcgcaggtggccgagcaggtgagtcccgggcgctcccaccagcgcggaaaccgcgggtccggacagctggaggcgagtcccccgcggctcctctcccgcggaccccgccgtctcaccgcgatgtcgccgctgttttccgcaggcacctgcgtgcgccatgggacccgcagcggcagcgcctgggagcccgagcgtcccgcgtcctcctccacccgcggagcggccgggctggatggaaaagggcggtaagtccgtgaggtgggggcttctaatccttgccatttagcagctcgtaatctccgtgccg

SEQ ID NO: 188 - DNA, 300 bp, Homo sapiens
  source 1..300: mol_type = genomic DNA
cggatcgaaactgtggtgaaagacctttggccgacggctgatgtgagtatgttctttggagttctgtgtcgcacgtcacgtgcgagtaaatttaaataccctgtgatgatgatgtgtcggctagatccacacagaccttttctcgttggcccgaggcagcagttctccaagtgtgctttgagaaggcctccgttgcctggaatgcatggcccccggcgcacctgcacctgctgtcttagacacctgcggtggccgcacatcttcgtgatgctggcgcgcactcaagtgcgaagaccatcg

SEQ ID NO: 189 - DNA, 574 bp, Homo sapiens
  source 1..574: mol_type = genomic DNA
cgggctcggcggcggtctgctcgcacttacgtcgccagcccagtctcgtacccgaaaattcaagccccatccgagacagggaacccagcaggcttgcactgccacggtggggagcgggacgcacggagcacgacactgactgggggaagggggcagcagttcgcggctcctgcagagcagctgcgtggcgggaatgggtccttccaccggcggtgcggcggccctgcgccggctccgggcagccgagtagcccgccacccaccaactagctaagcagccgcctctgtgaagctcggcggttccctgtgcgcctgcgaaattttgactccgactcaccagcgaccggccaccgagccgccgctgtaggagctgagagcacgtcttgaacaccggatctttccacccaagacccgacagcgtgcaggggcctcgagcagtaatttgaggccgcgtttcccgccaaggtttggccccagctaaccgccccacccatgcaaccgagcgggaagaaagctgtgattcgaggggccaggagaatacgggaaaagcttctgttctgcgcacagccagtgcg

SEQ ID NO: 190 - DNA, 1504 bp, Homo sapiens
  source 1..1504: mol_type = genomic DNA
cgcgcacagggagtcgagtgctcctttgtgccatcacctcttctcgaagctccaggcgcagagggcaaacgctgacgccctttcccgccttctctccgtctgcgagttccggagggcagtgcactgtgcagcctccgtacttcccgacacgttccgcctgcaaaccacttaggagcttctgccggcaatttctcggatgcagcgcggagcccagcctcccgcactgactcaggcccggcaccaccccgcctgctgagggcgtcgtcgcgggcgccaccgacggcgccagcctgcgttcctaaaggctccaggacaggtgtggggctgaaatcccgctcctccagcatcttcccctccctaccaaaagaaggctgctttaaatattcccggcctggagcagcagcagctaaggacctggaaagaggcccgaaaaggccagggcagggcgaacgaacgtgctgtccctatttaccggccacacgctctgactgcgggctgaccctgatcgcacacttcgagcctgttcactcgctgggcggaccctccctccacgctccgcgcccaggcaggcgaggtcacccactcgaagtcaccacgtttgagtagccctggttggtggggcagcgcccccttggccaacgccccgccctcctctccatcttccgacgctgggctctcgcccttcggcgtccgagagcccctattgctgcccagctggccccaaaagcgtccgaaagtcccctcttggcttatttcagagatgccacgcaataaagaaaaggcggctactttgcattccgcagctgctgggacgcccgggctgcgagcgcggctcctggattccagcctcccgcccttcccaggcgctggaatggacacggacgcccacagtggcgggccaggtagtgccggagtcgggggcccaggccgcggcgccccgcgcctcatcacttaccttgcctttagctatcaattccatgatgtagccaaattcactcatctccccagactccgacatgtttacaccccttcacaaactctggaggaccgacgcgggtgtatcgaatttgtcctttcttttctctttttctgtttttagtctgagttttgccgagctccccgcccataagctgttaaccaggaaaagaggggaagcgccggggaaagcaagaagcgggcttgggtgaaatgaaggccatcgagggctcccgggcagcctcgcccggcttgctaggatccgggtgcagcggtcggggctgcgcgatctaacggaacaaacgagggcggcggcggcggcagcagaggccgtgaaaccctcaccaggcggagccgggcgcgcggaggccccgcctcctcgggggtcgcggcagcggttcggaccgcagcggagacaaacaaacgcgcacacaaagcgggtccgcactcggctcagcgcggccccagctagacattccccggcctttatcgcatgccggcaggttggccgtccccggctctcggcgcg

SEQ ID NO: 191 - DNA, 305 bp, Homo sapiens
  source 1..305: mol_type = genomic DNA
cgcgtcccgattatcagtgcaatgtgatcacttgattttaaacgcaactatgcgtccaaatggctgagtgcaagaggtcggccggagatagggccgagtgcgctgccccaagctgcgatccctgccaagcactcacggacaatgcactggtttcctccagggagcgtcttccatccagggcagcagtaggagtggaatctggagccgcacacgttgggcctgtgatggacaagcgcggtcacgtaacagataggtagagggatgcagcaacatataaacggtcttcgtaagcaatgccccgagcg

SEQ ID NO: 192 - DNA, 645 bp, Homo sapiens
  source 1..645: mol_type = genomic DNA
cgccagtcctttgtaggcgttcatgcgcgagcgcgagtggctgaaggagtctttccgctgtttctcggtgcactcattgcacttgcagaagtagtcgtggggccgctcgatgcgggcgcccttgagcagcaggatgtgcacgatctcatactcctggcagtgcgccgccaggatgatgggcgtgatgtcgtgggagaagcgcgtgccgtcctcgtcgtaggcatagaagtcgtcgtcgcgcagctcctgttccagcgggctgagcgtcaggcgctggccctgcgcgaaggccgggtggttgaggatggcctccacgatgcgcacatagcccttgctgatggccagcagcagcgcgtcccccacccgtgccaggttctccttcttcagcagcagctccgtgacctctaggtgctcgttgcccacggccagctgcagagcgttctgccccatgtagtccacacagttgaagttaagggtcttggactcctccagcattttccggaccaccgggatgttgccatactcagccgagtccaggaagcgctcctcctcgggcgtcagactggtgcccttctcgttgaacatgtaggcgggaccccggatggcctggcgacggcccttctccctcagcgttgtgtgccggcg

SEQ ID NO: 193 - DNA, 312 bp, Homo sapiens
  source 1..312: mol_type = genomic DNA
cgagaaaccgcggaatatcggcttagagcctgccatggcgaatcggctacagcgcggggaccgcagtcggctgctgctgctgctgtgcattttcctggggacgctgcgggggttccgggccaggcagatccgatattcggtgccagaagagaccgaaaagggctccttcgtgggcaatatctccaaggacctggggctggagccccgggagctggcgaagcgcggagtccgcatcgtctccagagggaagacacagcttttcgctgtgaatccgcgaagcggcagcttgatcacggcaggcaggatagaccg

SEQ ID NO: 194 - DNA, 2404 bp, Homo sapiens
  source 1..2404: mol_type = genomic DNA
cgcggcgtcttcctgcacgaagcgacccaagcagccccagtccgcaccgccttggtcagcgggcgctcccagcacacacccagcgcgcggagcgactcctcccagaggacgccgagtgtgtgtgtgtgttgcggagattagggggtggcgggggggacggcggcagtttcttgggctctggcggccccacccccaccccggcagcgcccagcctccaggaacccaaggcaaggctagggcgcggcgtggtgagaacgcgcttcggctagtggggggagcccttgcgccccccgcacccaggccaccgcggggaatccctcctccccggcatctccggcctcggcctagggtcccccgcaagccggagccggcgcgagcggcgcagcgggcgccaggagggggcggggagggagccggcgccggaatcacaaagccgcgaacccggctaggcggggacattctcataaaaccgaccctctcgtggcgagcagagggggttttttgccctacgctcaatcctccaaggcctcagtgccgcccccagtctttcctggcagaagaaatagtagccgaccactgttggggaaactgaggccagaattctgacttacagagggtagctggggggaaggggtgcgagaccacctcgcctacaaattctgctcctatgcctgccgcgatctccgttctggctccagggcctgcccgcgcacacactggcctacagcggggaccaggcggagggggcgctcaggtgtgagacgaacccaggtgtgcgggacccaccaaggtgtgtgggacccaggtgtgctggagacccaggtggctcagaaaactcagctaaatggctggaagagggtcgcaatcaccgtcccaggctagcaccctctacaaaggaacctggggccccagagctccggggaggcgggatggcttggccagccaggtccagcttgttcccggaacaccagccggttcggcccgggagtgggggttggggaggcgccgactgggaagccggcaggccggcccagccttcccgctgtgccgatatttacaataataatgtcgcgattaacgtggcggggcgcttggggctcgccgaacgaccccggcggcttctcctgcctgaatctggactccgagagagaggaacggagtgtgtgtgtttgtgtgtatgcgctgcagggtgtaatgtaatgtgacactgttggtttaaggtgtgtgtgacacccagtgttaagatgggtgaagcagtggattctgtgtttgtgaccgtgaggcgctgctgtaggtgccaattcctgtgttagagtgtgtgccatcaagcgtgtgctgagatactgggttgtgtgtttgtgtgggtgcagtagaggggtgcaaggagacctgtgctgtgtacctatgttctggccaagaagtgggtaagatgatatatgctagggtgtgttccttaaaggaatgtggggggtggctaggttttgagggttcatctagtgaactgtggctgtgacactcgtacatgagggcacgagattgtgtttgtgtgtccacgtcgcggggtgtcacacccggttgtgttattgctctgtaaagccacgggttgtgtgtcggtgctgctgatcgtgcagacactgaagcggattctgagctggcgacggtgccccagtgggcgctggtgtgcgggaggtatgtctgtgtgcacatatgcagggatgtatgtatgtgtgtatatatgtccacggtctggccctctgggtgcccgcgagaagctggaagcggctgagtgagccgaggtagttgcccaacaagtgtgtatgtatgtgttgtggacaacggtccttgcttgctccgtcctccgggagacccccaccaaactgggggccgcgcgcttccccaaacccatcctccacgccgcgcgctcctggagtctccttatccccgtctctccgagcgcgcccaggtttgggacgcggggactagggttctgctcagcaccccctcccctccctcggcttctcgccgcggacctgggaggacggaatccctcagccgcatcccctgctatgggctcccagcagcccccacctcagccccctcgcgccgagctcgtgttgggcccccgcggcctcgctccgccgagcgcccctccctcagctcccgccggccatgaccgcttcgggccccaagccgctgctgcctccaccgccgccggatccttacccgcctccgggcagcgccggccgcccgccccgctgtccatgagccgccgccggccccggcctgggctgcggctccgcacggctggggctggagctgcagttcgggctccggctccggctccggctggctctgggcgcagcagcccggcggctttgcgtccgcgccgcgctcccgctccccg

SEQ ID NO: 195 - DNA, 3468 bp, Homo sapiens
  source 1..3468: mol_type = genomic DNA
cgccgcgcagagtcggccgaatccagcttctccaagggcgggcgggtgggatgatttgttgatttgcttgttttccaccaaaccgcagggcacctccaggagctccgggacaacttacaaatgaagacacagagcccccgtcccttgcttcagagaaccaaataacggaactgaatgatcgtttcttcagcaaagcctaaagacgcttgcgtcccgcggagaggtgcccacgcgcgactcggctgccccctcttcttgcgggtccgggtagggtagggcgggaacgcgggacgtgccctaagcgggccagccgagcgcgagacaggcgatgctgcagaagcctgcccgcctgcgggtcacacaccggccggcctcggctgcaggggttgcggggccacgggtcgcggggcgccgggagccgcgagcaggtgacggccgcgccgggcagccccgcgggccgcgattcccgagagcctggcgccaccccgcggaagccggaggaactgcggcttcttcccgctcccgcgcggaggggagagggaggcgcagcgcggaggggagagggaggcgcagcgcggaggggagagggaggcgcagcgcggaggggagagggaggcgcagcccgcaccgcgcaatcccggggctctggcgggagggaaggtgacttgcgtgggacgactccaagaggccaggcccggccttcgcagttcacccacgaggataccgcgaccaagccaccaaagcactctctggccaggccgagaccccttttcccccgggccccaagagaaaaaagcactttctcctaaaacacgaactccccgcggaggggcccggggctgcctggcacggggcagggtgtgcggctgccgtgcgggcgcgggccagagtcacgtggaggcaggcggagagggcgagggggcccccgtaaatcatcctgagggatttttggcccaagtccctgtttccgaaaggttgcgggacggctttgacgttgattatgtagctgttctcttttcaagtgcgcgcccctccccaccgcagcccgtctggtgtgcgaggattgtgtttgaaagggaggaagcatccttgaagtggtgggtgatggattgtctaaacccttctgggacgttctgggtgggggttttcgaggcagctcatcaataaagacccccctggagagaaggggcggctggagaccagcccccgccgcgcagccccgaggccccaggcctggaacacagtcctcggtgtggggcacagggcgtgaggggaggatgagctccccctgagcagatggagatttctccttgcctgggccgtttgacccccaaagccttgcagactggccgcgtgggtgccatggggcgatttcttgggaaaggagggcagcagaaaggcccgtgggctctcagcgccttggagtcaggggagcaggaaggggagtggaggtgcggacacgcctccagccaccgtgctcccctaggaggatctcccttcagcccctgtagaaccctgcatctcaaccaccctcgtgcatgtggtttaccttggcatagatttccaagccgctacagaaaacctgtccacctcccccaaggtggaggcgggagttgggaggcaggggtgctggaaagggtcacctggctcaagccgaccccaggtctgacgggcggagagcctcgagggccacgccccaggccagtggtgtcctctgaggccgcaaagccgactggcttctggaggagcagcagcctcggcctcagcctcggctgggaaaagacgcagtcggggccctccgtgctccaaacttgaactactctgggtccccacgaggaggaggctgccctggggtcagaaactccgacggaaatgaaggggagggggcgtaggttccctggcacagctcggcctggagaggcagcctgggggcccaaactcatctagggcgcgcccggagcttccccggcctctcttggcccatcgcgaacgagctcccctccccgactgggtcggcctcgggcgcctttcgccttcgcaggccgccggggttccgcggagggcttgtgtccctccctcccgccgccgcagcccgcccgaaggtcaggccgcatccggggcccggggcctttggccaggctgcggggaggggtggggttggggggctccccaaagcagcttctcggaggcggcgtcggggcgccctgaggtccctagtcggcctctgggcgccgagcacgttttatttatccccacagaggatgctggaggacaaaagaaatatttggacgtcagagctgatttatatttttggaagagctacgtttccttttctgaagagaagcgagcgcgccccatatcctgagctcggcggggaggacgagaggggagttttgatgcgccaggaagttcatctatcgcccaggcgctcaggacccggcgccccgccgagttcgccgccctcggaaatatttatttatttgggggaagggagggcagcggggaggagaccagccagacgggagaagaaagccctgcccgggctgggttgggacctccagggcgcttccccgcgcggccgcagccggccagcctctggtctcggttggagggcatcgcaggcgcggcctggcgacctccgggcgcctttggctggtgtcttccgtagccctcggcgcgcaccctcccgcgtgcagtgcccggagtgacctcttcaggccccgcgggcgcctcctgggtgggccccgcggcccctccaaccctgcccagcgtgtgtgcgcggcctagccatctttctccccagcctgggaaggcgggcgggaggaggggatccgaggccgggggccgcggagcaaaaggcacgcggacccctaaacgacccccgagcgttgccacagccgggagggcggagggaaggtgtgcagagctcgggggtctcccaggacgcagcttggctcctaccggtttccaaggcatctgcaagctctgtcctgcgacctaaagcatggggacgtcccttccagcagcatcagtgggcgaaaagttaacatgctgagggccaccggcacccacacttgcttccaggaatacttgcggtacaggcttcctgacttattttctttagccgctgggtgcccccccttcttcccccgttgggagacgcagaccgactctgttccaacctaagccccttctctctgccttttggacaaagcgggccgcctctgctctccactcactccccgcaaccccgcaactcgccacccctaaacgcagcgggctgtaggcgaagtcgaagaggtgctccgcctggtaccgcggcctgcgcactgggatcggggcctgcaggccgctgggctggctcctgggatcatcccaaggttttggtctcgcaaccgaggggacccgcgcccggcaatttgggaagtgcggaaatggggtgaggccggtcttcgcttgagggtctcg

SEQ ID NO: 196 - DNA, 2654 bp, Homo sapiens
  source 1..2654: mol_type = genomic DNA
cgatgcggattcgcgctggcgctgaacgctgagcgcaggcggggaggctcggcactctccgacctcagtagcgcccccaaaaccgaagtccagccggccagagaggacacttacgtgcacacggtcttgatgttcgtcttgtcgtccaggccctgcgggtagttggccatgctgtcgcccagcttgagcaaacagtccgaaaagcccttgaagaccgcatcgcacttgcccgctgctctcacggcctgcaccagatacgctgcggggaggagggaacaccggtcagcagcgccacgaccccgccctgccgcccactgccctttcctgcgagaccctggctccgcgcggcctcatcgcatcgggcggcctctcccagcgcccagcctcggggctcgccagtgttaaaggtgaatgaaagacgtgacccaggaatgcccgacctgcagctaacgctgcgtctagtgcaaattgtcggtgtgtgagcaggtgtgggtgggtgggaggtaggtgagtccattcctttcctccagctccctcccgcatctcgctccgtatctttttctgtttccatcgccccctcttctcacctccctcatcccttttatttctctttctcctcgctccctccttcgtctcccaacacttccccagcaagccctgtcacagctctcgcaatgcgatcttccattttgaggtgacagaaggggtctcggctgcagcgtgcgggggcgagaacggggtggggagacgacagggcgtggggtcacgcagctgaacccaacgccacatcacgagaataagcctcaggcttggcgctctgtccttggtgggcgctcagcaaacactgctgaatgaatgaacgaatatagtccctggaagccaccagcagttaccgaaatggattgtttcattataaaaccgattgcctaccgtggactctgtattccgagtgagaccctcggatgcaccctctcccgccccgctggaaagagggcagcctggacgtcctgagacctggcgcggatgatgagtggtctggagaggagaaggccagaggccgggcggggtcacggatgagtctgcctgggtcacctccgcaaaggccaaatccacgaacctcgggcctctttctctcctcctcccctgtccgccactcggctccccagacacccttaacaacccacccgcccgccaaaaagcccgcctacactccggacggcagcggggagaggctgttcttctccaggtgccagccctgctgctgaaagtgagctcgccgcggcccggggctcctggccccagctcaggactccctgcagagagcaggcagggataaaagctgagcggcgggaggaggaggaggaggaggaaacacaggccgcacgaaggtccaagccccaagcccccaagcccccggcctctggacggccaaaccccgaggcgcgggactggaaggacaggtaccaggctgcgggcgcgcggctgtggccatctctttccgccctgaggccgacgaacccggctggaagctgagtgcctagcggcccaaagcagcccgggcgccgggagggcgccagagaagcacagcgttagggcggggaagaaagggtgaatctcagaatcgaaatccgcactggcgcccacgaccctgggcgccggcctggtcctcggcagctttctggcggctgcgcttgtgtgtgaatgtgtcccgggaggaccggacacctcaatcccccggcccccaacgcgggcgcctgtccgcgagcgccgggccagacgccgaagaggaaggtgaccgaacccgtagcagcttccgagagcgtacccgtttgcaaattgctgcaggaagagcgaggcgggccttgcgctttttaatccggaacgggaagcactggggaagggaccgaggttaacttcgacctccgctggggcagatacgtaaaccttctcaaacctccgagttcaccttacagcgaacattaaatacccttgcgactacaatactaacaaatcgaacactgacgtaaaatcttaagaataaacgaatctttgttctgatagagctcgttcaaaatggaaatacactccacacatcttcctctataacgcatgtcacaaatacagctttgcagagaagaaacgaaataaaataaagtccaatctccccgtccacgtctctccccaccccttgcctacacccccaaaactcagtggcgaaagaagcccctaaaatgcattgacattttttaaggggaattggggctcgccgggggcgggagcgctcttgtcacgttatcttcccaggcgctgctggagaagaccctgcacaccgcgcatgtgtgagtgtgtgctgtgtaaaataagtcgtgaacatttctgtcttttaaaactgtgactccttggactctagccttctctgggaggccgggaggctgcgcgcgggacggattttgcaggcgcacgcgtcccagcaggtcgcagcccgtgggtccctcgctgtccttcctgaaccccgcttaggcgagactctagcgggtgaccctggctccttccctgtcctctctgccgtccccttcttgtgcggtttccatccgttcattcattcgttcgacctctcattcattccgaacttcctcctgctgcgtcttctttcg

SEQ ID NO: 197 - DNA, 527 bp, Homo sapiens
  source 1..527: mol_type = genomic DNA
cgtttcgggtgcggggtgctgatgctgcttttttgttgttcgtttgcgctcgcgctcgctctcgctctctctctgcatccccctcaccccctttctcggagactgaactaagtgaaaagttgtttcaataatcgcagctctctgctccgccagggccgagggaggcgggcggaacacggagggtgttttgttaaatgctcccgtcgttcgcaggggctgggacttgataaaaggagacagttttctgaaaagatttgattgaaatggcgtgtgccagggctgatgggagccagcgagggacaaagcgccgagaatccatggacactcgagcaattatgcctccacgctgaaggtggattagcgcgctggaaagaagcatatgtttggcccggggcgacacttccccccggctgagcttagagaatgggagcgcggagagcggctggacccggaatatcaactatctgcgaagcccccccttctagcccaactccgccagcctccccgcccccgccggggaaaagtcg

SEQ ID NO: 198 - DNA, 1121 bp, Homo sapiens
  source 1..1121: mol_type = genomic DNA
cgtccgagggtgggtccaacgggcttcgagtgttcgggagataatgcacacttcgcagtggatcttctcggggccccgagcgttgcccgcccattaggtggcgccggtcccctcgaacaggtcgctcaacaactcccgcccagcagccgcccttactgcgcgcgcgcagacttcggcgtctacttccggtgtggcccaggcggggtccgcagaaccagctatgtcggcctacggcatgcccatgtacaagagcggggacctggtgtttgccaagttaaagggctatgcccactggccggcgaggatagagcacatgacccagcccaaccgctaccaggtgtttttcttcgggacccacgagacggccttcctgagtcccaaacgcctgttcccgtacaaggagtgcaaggagaagttcggcaagcccaacaagaggcgcggcttcagcgcggggctgtgggaaatcgagaacaaccccacggtccaggcctccgactgcccattagcctcagagaagggcagcggagacgggccttggccggagcccgaggccgcagagggcgacgaggacaagccgacccacgctggtggcggcggcgacgaattggggaagccggacgacgacaagcccactgaggaggagaaggggccgctgaagaggagcgcgggggacccgccggaggacgcccccaaacgacccaaggaggcagcccccgaccaagaggaggaggcggaggcggagagggcggcggaagcggagagggcggcggcggcggcggcggcgacggccgtcgacgaggagagtccgttcctcgtggcggtggagaacggcagcgcccctagcgagccgggcctggtctgcgagccgcctcagccagaggaggaggagctccgggaggaagaagtcgcggacgaggaggcctcccaggagtggcatgccgaggcaccgggcggcggagatcgcgacagcctgtagttaccagcgtttccagaagagcccctgccccgttcctgctgcggcctggccgttcttggggaatctgaccacggcgtgcaaactgggactgcctttccctctcctcagcccgtcctcctccaacccgcgctcctttgccctgccg

SEQ ID NO: 199 - DNA, 268 bp, Homo sapiens
  source 1..268: mol_type = genomic DNA
cgccgcccaaaagcttattgagttcctcatcattgcggatcgctagctgcaggtggcggggaataatgcgagtttttttgttatcgcgagacgcattgcctgccagctcaaggatttctgctgtgagatactctaacactgccgccaaatacactggtgcgcctgcccctatccgctctgcatagtttcccttacgaagcagacgatggatccggcctacgggaaactgcaaacccgctctagaagagcgagacttagacttggcgcg

SEQ ID NO: 200 - DNA, 873 bp, Homo sapiens
  source 1..873: mol_type = genomic DNA
cggctttatcgtaggagcgtcttcgcctcccccgtttccagtagaaaagacagctttgctcctttgaaagcgcagaccgccgcacctccagccccttctccccggggaagtaggccccgctaagaatgtgggaaggtggtggggcggcgactgaagtcgcttccgattggcgttgtcccaaggaagcctgcgcggattgatcggcggcaggcctccaatagagcctgctaggcggattggctgctacgcggctgggccctgtttccggtacctaggcgggcagccatggtgaccggcgagcggcatgcgacgccgcctctgtggcctgtggaggcccgcttggcggcgctgcttcccgacctactggtctttcggaagcctcggggatgggaacccgagctcgccacggcccagggcgtcctcctaggcgtccatgtgacgggtgagggcgacggccggcacttgcacttaagtctcctggcctgcgggagaggcggccgttgggtctaagcccctggaggcctcgacggttacaggcctagcctcccgcgagctaaggcacagtttaacttcgtgctgcggcttccttaggaaactttgagtatcttcgttttagcgtgggaggacatcttttgggatgaaccggctcagtccttgagctcaccccaaagatgatgaaattgaaactcagaggaaaggatttttacaggaccacgccccgggctttgaacccgtttttcccaaccccgagcattttctattaagcgttctgtttcaattattttattcgttcagttaattcgttcttggcgtcataagtatatgcccgccgttgttctggccgtagctatctagcagccactccgcgagtcg

SEQ ID NO: 201 - DNA, 805 bp, Homo sapiens
  source 1..805: mol_type = genomic DNA
cgtccgaacagaaatcgcagccgcctcctgtgtgggcatctttcacggaaatttctgcccacgttcaatgcgtacgggtctctagcactgaggctagctcaactcttcggggcaggagaagttgcgcccagccaggaggacgccagccgcctccggggcccggaccgcgctcgccaaagcccttttccggctcgggcgagggctgacgcggccagacggctccgctactcgcgggcgaggcggactaggcggacgaggcggccgaggcgggcgaggtgggagaggcgggcgaggtgggagaggcggccgaggcgggcgaggcgcggggcgcgcggcgggccaggctggggaccgggaagcccgcggagcctcgcttttaagcggaatggtctgggacagccccaggtggttgccaggggaagggaggaagggaaggttctgctgagacgtcgaggttggaaataaaaacttcggaaaggcgaagcggatttagaaaaggagggaagtcgaggcgggcgaagcgggccatggagattgctaagaggaaaagttggagagaaaaggaaggaagagaagcagactcccgggtggcgggcgcgggccgctaggtcagacgaaaggtggagggatgcagaggctccccaaagagccgagaaagacacgcgacccctccggatcgggacagctcctgaaagcccggcgcagagccgcctcgaagatcctaagagtgggcgactcacaggcgcggccggcaagctcctgggggactcgggctcggacgagcgcccacaggcagcgcctggcg

SEQ ID NO: 202 - DNA, 554 bp, Homo sapiens
  source 1..554: mol_type = genomic DNA
cggcgccgttttggaaacccatctcggagcgccccactgcctccgcgggaaggtccgacgcacccggctgtccctgagggaggcaggatggagcaagggcaggacactggaggtggtgcccagcaactccggcgcgcgcttgcttcccaggctggaaaccttggcccaggctcaacctgtctgagctggaccagtccaggccacgcgccttggagccctgctaaccaacagggacagccgcgaccctaggtcgtgcgaacacctggggtcccggatctccgcttgcctgccatcatcccccaggctgcttggctgctggctcatctgcccgcaccgtccgccccgccgactgagctccccacaaagcacacagaggcgcagagccagcgctgttggagtcttttattgtgttctgtcaccgaggtcgcagccccgagccaggtggttggttaccagccaagcgagcgagtgcgcccgctcttcggggctcctgggtgccacccggaggtgtcctgttggttgcgctgattagagccgtcgccccctctcgggcg

SEQ ID NO: 203 - DNA, 369 bp, Homo sapiens
  source 1..369: mol_type = genomic DNA
cgccttttgtgccgggctgtggctcgctatcgacatctcgtccgttaccaaggctgggtttcctactgatttccctcctcctctgctttcacaggctcgcgcggccggacattgtgggtgtgcgtgctggatttctcccggatgctctccgactaacatggatgtcccaccattccttgcagtggaaggttgttccttggcgcagtgagtgaagaacatgcagcgattgctaatgggtttgggaagcggagactccttcctctctctatgaccatgccgtgatcgtgtctgcggtcaccactcgacgcatcctcatttctacccgaacccaggagccgaacgctagatcggggaagtgggtgccgtgcg

SEQ ID NO: 204 - DNA, 4868 bp, Homo sapiens
  source 1..4868: mol_type = genomic DNA
cgccgagttcgagaaagcgctacgccgccggtcgggctagctccacaagcggctgtacaagttggctgtcaaaaaacgctgatttctcctcctgtcacctaataaacccctacgcgcttatggcctcgtcccacaatcccccaatctcgtcccaattcgaaaaaccgaggaggagggaataaactgagagataaagatccccccatcttgctctttccccgggaccccagccttggtcgcggcgccccactaaggaggacacaggctctggtgtgtgtggtgtgcgagaccccgagctcgaggccgagccaaggctgggcagaaagttgcaatcacgtgctgtcggagcccactggagcgcacagcccgctccccctgggacgcccaggcggaggacctgctgcgccctcccagggctcgggggactccagcattcacttgcacgcacaggcgaactctgattgaaagcccgggatgacaccgagtctggagaaagagggaccggggggtgggctggcggaattgcagagcgccggccacagctcccctccccgcgaacgtcgagcggagggcgggaggtgtaacctctgacctctggccgggtccacgccctgaggagggactggcaagctcttgttcgacaagttcaagctgccgagagagcttaaatagaattaatctcttagagatcggggatcatcgctccctcggcatgcgctctcccagcgccgcgcacagagcaaggcgcgagagagctcaggaatcgcgggaaggcaagcggaatggggagggggtaggggatgagggcctctcttcactattcctccgcccggagagcgggagcccgcaacgcccgccgaggacgagcggcgggagggaacgctctgccctccagccgccccggtgcagataatggaggcgacaagagattcgctcagcgtcggatgggccagctctgcttggggaagctggcggcatcctcccctcggctggtgcccaaacccactgcgcgaaggccgaaggaacgcggaacctccagaagaccccatcctcagccctgactttccgtagatatgtgcaaaatgagtaaattactcacctcgggccagatccaagttttacccaacagaaggggcaccggaccaagaatgaaccaactcacatggccatgtccggcgcgcacaatcacacgccagcacacagccacccaatttcttccgcgaatctatctggcactctggagagagggggaaaagcgttttgagaaagccccgtcacccctccccttccttcttgccgtgaaatatacgaattcatttttattacgagccgcaccgtcctcaccatcacgcacgcacagagccacactcccatattcacactttctaactcgtaagctccgacagcgcctgcattttctttgggagccgcttggaggttcattaatatcattagcatttaaccccctccctcttcccatcccctccccgcacatggctgacgtcagaccccgccaggagttgggggaaaagctaagtgggccagggacgccctattcccctccccgcggctgcctgtcagagcgcttctggagatattacaggggacccagcccgcagcgacaggcacaaagtcacggggtaatgaacttcggggacccttcgccgctgcgtgcgcggctctccccggaaacccggacctggccgcctcttccctcggaagatttcccagcaatctagttttcccactctgcgcttgggttccggcagcgcggagcccgtctgcctctgagactgcggtagtgttttccttctttccttgggagaccagcggtcggcagagattgcccacactctgcatgcctatgtagagggagagatcgaagactgagtgacaggaatggggaaaaagagggatttcgctccgtaggaaggccattttcgtgtctccatctctgtctttcaacatccctctcttgctgttcttccttcttcctcagtcttcctgtccatctctccatctgtctgtccatgtgtgtgtccatatcaagcagcattcccagcagctgcggttttgcaagagccgggaagaaacttaaggatgcttaaatttccactgttggacgaattctgagcgcccagggagcagcgcagcgcgcgactgacacccacctgtcccgcccaggagccttgcaggctggagggcagctggagagcggcggcgcccggcggcgaggcgggcgctgccggccgggactcgggcagcgcccaccaaccgctccgccccgggacagccagcatgagcaagccagccggatcaacaagtgggtacctctcgggccgccgtggggcctaggcgcgcagcctggggcgagcgagcggggaggctgggggaggtcctgcctggagcgctgcgaatctgagcccctgagagggattccagcgggcgtgtgcgttcggcccagacctgtagaccgtgagttggagcatttcgtggagaggggagagccgtttcgttgcctctggattgcttgatcccccctgtctggtgcggtgagaaggttacgacccgcgcagcccaccagtcggatgagttgtctccatttagccgccaggtgctggatgggggggccatgggggcgggaactgggccgcagctccaggcggtagcacaataacacactcgctcaaaactccgagctccagcgcgcaaaagcaactctgtgcaaagcggattttgaatggaatgctttgcaccccgtttctagctatttcaaataatcctgcaaactgggaagcagaaacaatttaaaagtcacattttccttaatcctaaatccgcgtaggtcataactggggaatttaaagtatggcgaaccactctagcaaagagaggaccaaatccctaatcccaaggacttttcgagccggagcccagcagaggcaggagtgcgcggcctgctccctccgtgcgcttctctccttcctcgaacttccttagctgccggctctccgaacgccaggccgcagctgacctctcaccaccccgagactcacgagcgcagggctaagtgtgtgtgcgagggcatttgcttgcaccctgcctgcggaacccaagaatgtgcaggcccgagccagcgttgagcaggcgcggtcacggtgctcagatctcccgggggcatttcagttcccgccatccagtggcccacggctgcgggctccagggtctgaggctggggactaccgttgccgccgcagtccccatatcccgaagttgccttgctgcttgtgttgttttcgcagatagcatttttggcgctctgtgcgttccttccctccccctccccctttcactcgccctcattgtcctgagtctttgaaagttgggagaatcggagatacttctgaggactggtaatgaagtctcacttaagtgggatgcaattcccgccctcctacccccctccaagaaggaggttgtgttttcattttgttttgctttgggtgctgacctttaaaaaattagagcaaaatgaacgtgaacaaaaagaaaaggagaaatgtttcgagctggggcagagggagcagagaaggagccctcaccgcggccggaatgcagagcggaccctggcccaggactgggtttccctttaggctcgggcctaccctggccctcgctgttggaatctccaggaggtaaagcgacctcgatttttgttgcccgcattcccgggcgtgagtgtccttcccaggaggctcaggaggccgtttctgttgcattctgagcctccgttgcaaaaactgaagcccgtgggtctcggcaggcctcctagctcgctcgccccgggacaggccctcgcctacacccctggaagtaaggagccccgggctctttcgtccttttcggggtgtggagcccctggggcccttgaaaggtgaggcctcagaggcgagggaggggtgagcggggagctctgcccgcctgcggctgcgcccccgctgtggactaggaggcaggccaaccctccggactttgggggaaaaaccacagcgggctccttgcggaaactttggccgttctaacttgccaagagcctgagtgaggccttggaagcctccagccccggctcaggtcgggacgcggctgctgagctttctcaggcccgcaggacagcggcccccgccggtggcgccgctgcatttaggccctttccagaccggtggcggcagccaacccgagacttgcgtccctcgggcccggggcagctaggaggtcggcgcgcagcgggccgggtcaggactgggtcgagcagacagagctgcagcccccgccttgcccggcttctcgcggctggagagcagagcgatgtcacccggagccccgcctgggtggtaacgagaccctggccagtcacccctgcagcccagactaacttctttcaacagcctctgatggtaattacagtaatcgaagctgccatatatctttaggcaattatgacacacaaaaagccccgaggggaccccctggcgagggaagttaagaacggttttccagcttcaggaaactccggctcgcctcacgtcggagctcgctcggcttgctaaatgagaggagctttgcaacggggtcaaccagcttgtctcgtgaccccaagtcaccttaacgtggctgggtggcggagtctgaggcacaggcccgctatgccccggaattttcgcgtccctccctcctgggccccgccccagcccggttgcctgtttctaatctgccccgggagccgcggctcagaggtctgctcagaggcaggactcgcactggtggtggcctagagggcaacagtccggaagctcgggcgggggaatccg

SEQ ID NO: 205 - DNA, 497 bp, Homo sapiens
  source 1..497: mol_type = genomic DNA
cgcggtacctgagctccgtggaaggcctggactcctcggatccgctgcgggtgcggcttgtgtctcatctcagcacttgcgccacccagcgggaggcggcggccatgacatcctccatggcccaccaccatcatccgctccacccgcatcactgggccgccgccttccaccacctgcccgcagccctgctccagcccaacggcctccatgcctcagagtcaaccccttgtcgcctctccacaacttcagaagtgcctcctgcccacggctctgctctcctcacggccacgtttgcccatgcggattcagccctccgaatgccatccacgggcagcgtcgccccctgcgtgccacctctctccacctctctcttgtccctctctgccaccgtccacgccgcagccgcagcagccaccgcggctgcacacagcttccctctgtccttcgcgggggcattccccatgcttcccccaaacgcagcagcagcagtggccgcg

SEQ ID NO: 206 - DNA, 3450 bp, Homo sapiens
  source 1..3450: mol_type = genomic DNA
cgtcgcctcccgttcaagcagcgtcccttcccacaacccccgtgcagaaggcgcagcgcggccccctcctcgctggtcccagacctggcgggctcctcacacctacctggccccctccgttgagcttgttggtgagcttgactttgctgaaggagacgggagccttcatccagtgggccccgaagttgggcgagtcggggtggatgtagacgcagctgggcgcctgcggctccggcttgccccccggcacccattccccgttcacgtacttccagcggtggttgtccgccgccacgaagtccagcaggaaggagtacatggcgttggggtccaggccagacacgttcaccttcagcaccggaaacatcctcctggaaaacacggggcgggcgcaggaggaccccgacactgaccaggtaggccggaggcagaagctgggcacagaggcctcagttatttcggggcacagaggagccccctggggaacgtccgagggtgactcccaagctcccccttccccgagccctgccaggtccaggtggcctcctcaccatctccacggcctcgggagggaggggaggagaggacctggcgaagggtttcagtgcaggaggccacatcccgcggggacaggcggggaccagggcgcgcctcgcgggtcccgggatgcctccgaggtcgggacaccgaagtgcgcgttcctcctccccaagcttccgcggagaaagccccagaagaggggcttgtagaaatgcactcgagggagttaaccagagcgggaacaaacacaaagccctcctccaggagaaaaagggttcgacctccgggaacttgccaggtcccccaggctgcccaggcgctggagagcgcggcgcgcgcgggctccggacgcgcacccacctgccgttcttggtcacgatcatctcattggtgagctccttgaagcgcagccacagctcgctctcctccaggcccacgcgcagttcgcgctctgtggggtcgcccttctcgctgcccgcctgcagctcattctccacggcgctcagcaggtggtccactcggtactgcaggctctttcccgcgctctcggtgccaggggagctcatcctcccgtccggctcccctccccgccgtccccgaagcccagactcgctacctgagatccaccttccctgctcttggccgccgcccttccgagaaaaggggccccttggaccgagacctgcgacggctcccgggtcccgggtcccggcacagacccgggaggagggcgcggaccaagacttggggggaggggacgggggcagaggggtggggagaagttattccacttgaactccccaaggctctactagtgtaggtctctggggaccgaaattcagtgccctccccataaatagagccgcggcggcagcgctggggtgctcggcggattgggccgcgcacgctttgaagtgccgggcagcctcccattggccgagagcggccatatcagacccagccgggcgggtctgggaggccgggggccaacaatgggctcccgcacgcctgtttcatgtaaatccggggccccagccctgcgagccccggcccagcccctacacccccgcccttctccaaatgtttgcacctccatcaaagcggcggggcaagccccggaggaaggtaagctggacgccccggcctgccccagcccgccccagcccgccccagcccgcacttccgcgaggggctgcccggcagcctgcgatgctccctgattcccgcgcggggtcctcctcgctttcccagcgcgcccggatcccgcagccgcgccaggctggaacgttcgcgcggcctcacctgtccccgctccaggcgcgcctccctcccctcccagggacgagcgcatttggccgggctttgggatgcgccgcgctcgtctcgccgcagtagtagtgctgttctcgcgcctccgatgcctcaactctccagtctggcagtttccggtgcacctgtccccacacgtccctcgcccacggagccccaggcggcgttacgcacacccaggatcgtggatcagcctgccccggcgtcgggtgtccccgcggctctcaccatctggaaaaggaaggtccgcgcgcagagagggaaatggacggaaataagcaaaagcaaaacaaccccttctgagaagtgtcctcctcgctctcttataaaaacaggacttgttgccgaggtcagcgcgcgcatcgagtgtgccaggcgtgtgcgtggtttctgctgtgtcattgctttcacggaaggtgggagaaggtcggggctggcggggtggtccggactagtgggactcggggcgctttccccgcccggcccagcgccaagcggtgtcggcagcggttgttttattacctttcggtgagaacttacgcgaggagagcgaaggagaggtgacaatgagcaggaaatagttcagtggggtctctttaaacagtaacattcctcctaaaacgggagcccaggaagggggaaggaggcagaagctcccccttccaaggcctggctttgcgcttctgcacacctcgcccattcgctggacgagggcctgcctcgggtagcagcctctgtttcccggtctcctgagcgggcaccccagccccaacccctctagagaaagcctgcgcatcgggtggggcagcgagttcagaggagtcttgcgccctagcaagggggctttcccgcggacatggtatctgcgccccgcgctaagtctcccctgccccctgccccctgcccctcgcccctcgcccagccgggctcgctccggggtcgggaattgctggggaagccgaaggagggatccccgcccgggactcgagggatcgacccgcctcgcggtatgcggagggctgccaggagccagtcccggggaagggtgcctgccgcccgtgggggacagggctccttctctcccttctccaacttacaccgacgagtcaggaagccggagcgtctagtccctgtccgcatttctcagtgtcttttgatgtctgggatgcatggatattcccagggggctgacccgaggtggggaagtttccttctagaaagaggcaccaaaaggacctcatttgctcctgacgccggaccctcctgacacgccttcactggctcatcgggtcctgggctttgggggagagcgtggtgcgtgttactctccttactccgggtattaaaatgtctaagaggtcatcgctttggcccaaggcctcccgaggataagataaaagggaaaagtggaggggaggtgaaggaggaacgagggtgggtcccaggcaattgagggcctcggaaccctaggcacgggagcgaaacctctcctgcggcttcagggcaaaggagtcccgaaggctggatccctcccctgcgctcggacggcgtcgcggccgcttttcccacagtcgggaagcg

SEQ ID NO: 207 - DNA, 349 bp, Homo sapiens
  source 1..349: mol_type = genomic DNA
cgcggccgggttgatctccaacacggcaatcttcccgccaaagagggaggccagcaagcacagcacctgcagggacgcggtttcaacatcgttagcgacaccacccagtaaaagcagacgaaacgtgcatccgcacacgtatcctaacagctgcttcattctcatagcagcgacatcgccctgtcactcctaaaacgtttttacacacaaaatcccgtttactcagccagtcgttacaggatcactctgaagaaagaagaaatcagaggcacgtctcatagacagggagggccgcgtctcaggactgaccttttgatccgcagtgcggttcctcagggcacacgatccg

SEQ ID NO: 208 - DNA, 470 bp, Homo sapiens
  source 1..470: mol_type = genomic DNA
cgcatacgagtccaggggctgctacaaagcgcggcgacgtctccgcggcgagctggccactccatggtcccggcgcagacactggggcaggcgccggctctgtcgtcgaggcgctcacaggcaggcacacgtgagctcctggaggacagggagagcggccgccccgcccctgcggagcacaggacgcttcctgccacccctgcagagcatgggacgcttcctgccatggtgccgtggcaatgggtggcacctgcctgtggccccttctcagaaggacgttttaaacgcgtgaggtctgatgcacagccacagggagacacagacgagcagatgtgggcatccgagtatttacaaggtctggtggctcctgcagccgcgacacgggctgagcgcaagtgatgtgtgaggtgtccccaacagatggcacggggagcgcccacacccgccaccgcggggtctgcggaagctcg

SEQ ID NO: 209 - DNA, 385 bp, Homo sapiens
  source 1..385: mol_type = genomic DNA
cgcttctaaacccaatacgcttttctggagtcctcggaacagctggccggggcttttagcacacgggacgggaccttcgcctttgcctgtttttttttcgaccaggagaagcaggtgcgagagtctggggcttaggaggtggaaggcggggaaagagaatccactaggtcctcgtgtagagaacaacagtcgctccttagatattactccaggacggaaacctgattgcaaaccgctgttccttcgaaacttgcaaaacccggaacagaaaactcccgcccagccaattttagctctcgctgaactctcccgcctgcgttacgtttgcacagcaactctttgtaaatgtccgagtcctctcggaggaaagatcgttagggcgacg

SEQ ID NO: 210 - DNA, 2949 bp, Homo sapiens
  source 1..2949: mol_type = genomic DNA
cgagagccgcgtccccgcggtcgcgtggatttagaaaaaggctggctttaccatgacttatgtgcagcttgcgcatccaggggtagatctggggttgggcgggcggcgccgggctcggctcgctctgcgcactcgcctgctcgctgctggcaggggcgtcctcctcggctccggacgccgtgccaaccccctctctgctgctgatgtgggtgctgccggcgtcggccgaggcgccgctggagttgcttagggagtttttcccgccgtggtggctgtcgctgccgggcgagggggccacggcggagcagggcagcggatcgggctgaggagagtgcgtggacgtggccggctggctgtacctgggctcggcgggcgccgcgctggcgctggcagcgtagctgcgggcgcgctctccggagccaaagtggccggagcccgagcggccgacgctgagatccatgccattgtagccgtagccgtacctgccggagtgcatgctcgccgagtccctgaattgctcgctcacggaactatgatctccataattatgcaactggtagtccgggccatttggatagcgaccgcaaaatgagtttacaaaataagagctcatttgttttttgatatgtgtgcttgatttgtggctcgcggtcgtttgtgcgtctatagcacccttgcacaatttatgatgaattatggaaatgactgggacatgtacttggttccctcctacgtaggcacccaaatatggggtacgacttcgaatcacgtgcttttgttgtccagtcgtaaatcctgcctgatgacctctagaggtaaactcgtgcactaataggggagttgggtggaggcgaggggggtggcgcgcgcgccccgggcgcgtgcccgccgccagttgccgccgttcagccggactcgagcgccacccgctggaggcagggctcatcgcccagcttccgaccgggggctgcaagggccggggtcgaattgaggttacagcccattatggcaaaattattgcatttccctcgcagttccattaggatgtaccaattgttaggccgtcagctgccgatcgcgcgcccggcgaggatgcagaggattggggggaggtggtgacttgcattttatttacaacaactttatttcccccgttttgcagcccctcttatttttgtgtcgaggttggggtcggtactgaccgtcctgccagcagctctgaattttgaaaatacagatatcaccttcggggaagggggaaagccatttagccaattggagaaataaatcctgcccgcagcagcagcagctacaattacggctctgtttttgcgagcgcatgagggacagtgtccctgccgctcttaaatgacaggcgtctattaaagatagcttttgtgtagtgtttctccaaggcgaggtcaaattccatacacttttataaccgtagtcgatttttctttcgtgtgaatatggttttcgtgtcattagtttgcgatttgatttgcttacgtatccagcctggaaaatcttcatcacagggtccggttcctcgagccagccgggccccaagtcggagggttctccttgaacccagcgagtgggcccaggctccctgcagccacagaggctgcctggggtctggggatccgtggggcgggttactggggtcttgcttagacctccaggagtaaaatgagggcgataatggaagcattccttggcagtgcctagtatctctgtagttattttccacggctccgaaagactcaagtaaatcacaaatatagctgagaggcaagtggagtctccccgctggaggcccggcgttgcaggcgcccctggcacgtctggaagccaggactctggcggctcccatggccctgggcccctcgttgggtcctgaacgctgctgtggcggcgacgcgggcgctatcggaggctgggagcgggaatccggagccgggagcctaccccgggctgtaatgttccacccgcgcccaggttaactcgcctcggctgaggctgcttctcttccactgacggttgcacacgcgggaccgagagactgggctctgttggggccccctttgttcctcgagcttccttcctgttctgggaggcggcttgggaggccgcgacaaggccgggctccagctcttagaccccctctttccactggccagagatgatttgatgatgcccttcgggacttactggcgagggacttaggcagagacgcccagacacgaaacggggctcggcccagggctctttcctccccagcagccccgcgtcccgaggtcggggagctcagagacactagcacaggagccccagacgcattcagggcgcaccccagaactccggagccggtttgggcatccttgtggagcgggactgggtgtgtgcagtgcgccccgctccaccgctggtattggctgtgtgtgaggttttgttttgttttgttttgttttgttttgttttgttttgttttgttttgtaagaaataaatgcacagacgcttgcaaagctccgggctcccctgaagctgcggaagcccccagatgggagcaggcggggagaaaagttggggaacaggcgagggcaagggggcaaagccgaaggaggttgcagcgctggcctggtccctgcccaggcatctactcgcccgcctttgcctctgagtcctccccgctgggctgcgtggaattgatgagcttgttttcctttttccacttcatgcggcggttctggaaccagatcttgatctggcgctcggtgaggcagagcgcgttggcgatctcgatgcggcggcgccgtgtcaggtagcggttgaagtggaactccttctccagctccagtgtctggtagcgcgtgtaggtctggcggcctcggcgcccatggctcccatacacagcacctacgagcagaaacggccgggcgccg

SEQ ID NO: 211 - DNA, 2547 bp, Homo sapiens
  source 1..2547: mol_type = genomic DNA
cggagctgggcaagccgtcagggcgccctaaggccgctgatcacgtctgtggcttatttgaataatctgtcatggggacccttgtggcccgggtcgcccgcagcctcatcttggcaggatttacgccgccactggccgaaggcaagaagtggaaggaatcggccgtctcccccagcgtcccagctccggctgccctggctgccgccgctcacggacaatctagttgtacaaaaggctctctgggctgcactgctttcgaagaacggcccaaagtatctcggtcctgggcctgggcagccaaggagaggggcggccagtcttggctcgtcccgaagtgcccgccccgccccctctcgctgcagcagccgcctcctctcccgtagccctgcgggccgctcttcactgctctccagacttggggccctatctgaggcgtcccaaacaccaacttctggctcctggccccaactcgagaggcttccagcgaggacgaaggcaggctcgagagaaacctggcgggccagcagatccgggaggccggcgtggaggcggcggcggatttgaagggaggagacacttactgggatcgatggggggcttgtctccgccgctctcattctcagcattgttttcagagaaggcgccttcgctgggttgtttttctctatcaactggaggagaaccacaagcatagtcagtcagggacaaagtgtgagtgtcaagcgtgggacagtcaccccttctggccgacagcggttcaggtttaatgccataaggccggctggagggcaagcccgcgaaggagagcgcaccgggcgtgggctccagccaggagcgcatgtacctgccgtccggcgccgccgccgccacgggcgcctgggggtgcacgtaggggtggtggtgatggtggtggtacaccgcagcgggtacagcgttggcgcccgccgcgtgcactgggttccacgaggcgccaaacaccgtcgccttggactggaagctgcacgggctgaagtcggggtgctcggccagcgtcgccgcctgccggggaggctggcccagggtccccggcgcatagcggccaacgctcagctcatccgcggcgtcggcgcccagcaggaacgagtccacgtagtagttgcccagggccccagtggtggccatcaccgtgcccagcgcctggcccgcccggcccgacccacggaaattatgaaactgcagatttcatgtaacaacttggtggcaccgggggggaagtacagtcacctaataagttgccggcgcccgcgcccccattggccgtgcgcgtcacgtgcccgtccagcagaacaataacgcgtaaatcactccgcacgctattaatggtccgatgttttgcagtcataatttttatagcaaaagccatatgtttttatgtaaagggatcgtgccgctctacgatggggtttgttttaattgtggccaacgacgattaaaagatcaaatctagccttgtctctgtactctcccgtctccccccccatacacacacttcttaagcggactattttatatcacaattaatcacgccatcaagaaggcgcgggtcccgcgtgcgagtgcggccagcggagcccctcacataaaattagacaataattgaagccataaaaaagcagccaaatcgcattgtcgctctactgtatttaaatctatatttatgatatttcataaggagttattgtttcagaagccacacaggctggcgggaagtcggaaacgaccaacagattcgtttgcctcgccgtggctcccagctgtaaaaatttacgaggacttggaaaggttagactgttgtgtttggttggcgagctccctgtaaataatccctgcggtccccgggagaggcgagtttacccgcggccgccctcgaaaagtcaaattcaacgcaggatccgtcccaaacggagccgccgccggccctaccagggcactccaggcagggaccggccgctcagggagtaccgcgggtgtaggtccccacagctacccgcctggagcgaggggcgcccgggcaacccttaaattcgcctttgctacgaggaccccacggaggagctggccaggagggagcggccagccgccaccagggcgaaggttttgagggcctggttggttgtgcggcgcgctcggtccccggccctcgaccccacgcacacgcgcgcccagcccgcctttctcatcagctggcaatcaggattcccaggcgcaggcggctggcgacccagccctgtgctccagcctcagaggctctaaccatgagcgctgcaagcctggttgcgctccgtgaatcccagctggggaaaaaactacaagtggcatgaatggaaggcaagttcggtttgggaaaaggcagcctcgcctaagagaccccgcagctccggaacctgggaggcccgcaccgatgtggcctgtcccggggccgcgtgagcctttcagggctccttcctccctttccagctgctactccgggcctcgccttggttacctacggggcccggagactcggcg

SEQ ID NO: 212 - DNA, 1292 bp, Homo sapiens
  source 1..1292: mol_type = genomic DNA
cggcggcagcgatcggccctgaccatagaggcggccgtggcgcggaggacttgacctttacggcacgagaggagggcgctggctgagccgcagggagggggacgctgctctctccagcctcctccacccaatagcagtccgctcgacccacgccagctgcgcgctcaccccctttccccctccattttgtgcagtgtccgcagacccgcggccggaaacaaagcccccagccctgctctcaccacccttcttgcaagctgctgtcgccaaccccccagaccagaggctcagagctaacgctaagccccttaaggccctctccagggccctcttctctcgccagccgccctcagccagctcctgaccccagccgccctcagccagctcctgggtaccactctgggtgccaaccgggacctgccggtgccacggtcccgtgcgccgggtcccggcctcgggctgcgggcggcttcggggcctacagcccagcggcgcggagcgtggcagaggcgcggggcgcagcgcagcccgaggctgcctcccgcccgggcggacggcgcggccgctggggtgcacagcgtagccccgcgtgcccggctcgctctccgttccctcggatagctcccaccgctcggctccgggctcagaaagcggaagtatttggtagagaaaaacacggtttcttttcagctcgtctcaaaatcccttttagagaaaatgctcctgtcaagttttatttcccgttgcaaaccaccttccacgctgccaagaattaagaccgggagagattaaatacccgattattctcctggggagggcggggcggggccgggaagtgggcacccacaccaaacattccttgaagtaggcttgtcctgatccagccgcgcccggggagccccacgaaggcccgcgcggcctgcggtgacgtcagcctgcagttgcggggccactcacccgcacagacacggcccttgctgttccgcgccgggacctccgccagccgtcctgccgcagccccgactggcccgcgtgccgtcagagggaggcccgctttacaccggcctgagcctccattttgagtagggggttcttgtagaagcgtggagggtttggggccgcgcctctgtccccgaggggcgcgacatcaggtagctctccgagttcacaccccagtacctgggggaggacctatgtcgccgcataaccgcccgcagaggtttggaaatctctgagacccgttgtaattatttctgcccagtggatccggctcttcagcgtcacgagagccg

SEQ ID NO: 213 - DNA, 1297 bp, Homo sapiens
  source 1..1297: mol_type = genomic DNA
cggtttccggcttttggacaatggtccctcgcttaacgaatgctaacgaatgctcccttagctgggagctgcagggaccggtcccggcaggtctaaccaaccgtccgcaggcgccgtggtcgagagcagagccgagacggcggggcagggtgccaagcggagggcacagcctggcagccaccgttggcggcaggagagaatgggagaagggagacccgcttctcagcacaagggcatctgtctactcccgaacatggccggagattcctgtccacagcctccctccagctctttctggtagacactttaaaagcgacattaacgggcttcggtgcctttggattcctaaattaggtttacggaaaaggaaagacttcccaagaacagtgagaagaggaactgcagcccctccaaggccgctgcggcgcgttcccggggcgcgccgagagcagcgcgcggctccgtgccccgtggggagcgcgcggcgcggccttggatttcaccgcgagtcgggagggcgggtctgagccttgcctcccaggatccttccgacgaacaccccgcgggttttagtttatcgagccaaagtggtcccggagaagcgctccctcgcagccaagctgcaagaagtggccgggaacctacaggcctcgggccgacccaggaagcctccgcaccagaaagctcgaggagcccttacccaagtcttgctcgaagggcaaagcaaagagccagcacccctgagtgtcactgaagttcctggatggggtgtgagtgcgcgcgttccgtccgagacctcagtctcgcccagctatagagccgataaagggatgtcttgtgggcgtaaggcgcttcgcgcccatctccaaggccgatgtggtcaggaggtgaggggaaatgtccttctggcagaagcccgcggtgctgcgacgttgacccgcctggcctcaggctcagggcggcgggcagcccagggcacatgtagtttcagcagccgcgctacgtgggcgggggaccccaggccaccccacgtgtccgccctgggcctcctccgggtcccaaggcgcggcgcctccaggccttgtagcgtcttccccgggtccccgcgcgccaggccccgcagcctgctcacaggacagccctcggggctgcgaccctctcgctcctccccgcggccactcgctccccgttcatcccccacgagctcactaccgcagggaccggccctcagagccccgggcctcctcccggcagtggaagaatcagcgtgctaacattgtgtgcaaaactcgcagcg

SEQ ID NO: 214 - DNA, 417 bp, Homo sapiens
  source 1..417: mol_type = genomic DNA
cgagggccttgcccgccctcctcctcccacccgcaagggttggcccgcgttcccctgcgggcgagacaaagaagcaggagccaggacccggctggcgcctaacccggcgccggggccttgagcccctgaggtgtgcgccttcctgcaaggccccaaagaacccgagtttttgccccttgctgaagggttggaagtgcgggctggactagtgcaaacaagagtgactccaaccgccttacttcagctcattaccgtcacccatgaaaacggaatgaaggactgggaagccaatctgccgcgttcaattggagagaaatgactgtcggaagaggccctgcggtaaatagcctctgagccgcgaactctatgcgccgtggccggtggcagaggccacacctggcagccactcgtggcgcg

SEQ ID NO: 215 - DNA, 423 bp, Homo sapiens
  source 1..423: mol_type = genomic DNA
cggaaatcatcggcgactgctgttatgaggagtacaaggatcgcaggcgagagaacgccgagcgcctgcaggacgacgcggataccgacaccgctggggagagcgccttgcccaccatgactgcaaggcagagggtctggagggccttcgagaacccccacaccagcacgatggccctggtgttctactatgtcacggggtttttcattgccgtctctgtcatcgcgaatgtggtggaaacagtgccgtgcggatcaagcccaggtcacattaaagaactgccctgtggagagcggtatgctgtggccttcttctgcttggacacggcctgcgtcatgatcttcacagttgagtatttgcttcgcctggctgcagcgcctagtcgttaccgttttgtgcgtagtgtcatgagtatcatcgacg

SEQ ID NO: 216 - DNA, 454 bp, Homo sapiens
  source 1..454: mol_type = genomic DNA
cgtcctccgacgcgagatggctgtgtgccaccgactgtccaaatgccctgccaatgccatcgtgaagcatccgtgtgaaggaaaaagtccaccttaccgtgcgctgtggcatcgatcaggcgtgtggacagtcgcaggcacgcagcggtctcccaacatcagggcacaccgcgatcaggtgtgtggacagtcgcaggcacgcagcggtcctgcagcagcagggcgcaccgcgatcaggtgtgtggacagttgcaggcacgcagcggtctcccagcatcagggcacaccgcgatcaggtgtgtggacagtcgcaggcacgaagcggtcctgcagcagcagggcgcaccgcgatcaggtgtgtggacagttgcaggcacgcagcggtctcccagcgtcagggcgcaccgcgatcaggtgtgtggacagtcggtgggcacgcagcggtctcccagcg

SEQ ID NO: 217 - DNA, 273 bp, Homo sapiens
  source 1..273: mol_type = genomic DNA
cgcaggagggctcggcctgggaccccctctgcgggcctgcggctttagccagcagaaagaggggcgtcagtcctatccccaaagttccgcctcgggaaagagccctgagctctgaactccgacgaaaaaaggaagggctctgttccggaccgggtggggtttgggaagggtagagccggcctcgggtgcccttcttacagggcctgtctcccgctgcgtgcaccaagtgctttgaaagtttttcgcggattaaaacgaataaaaacctcgtcg

SEQ ID NO: 218 - DNA, 3360 bp, Homo sapiens
  source 1..3360: mol_type = genomic DNA
cgagggtgaaggatcgccgcaaggcaccgcacctcccgctgcagcccatcccgcactactaggagaagccggcgtaggagcgccgcctgtgtccttggctgtggggaggacgtcagatggcaccccgccagacactaagccccaagcccctggcttgttgctaagaaaattcactgcccggtccagactcagcccttttcgccctttaagggtcgcgcgtgggaggcagctctgagaccccgggtagcgctggagccacagatttcctccgagaaaagaaaggccgggatagcttcccgctcgcccaagcccagattttccactctccaggaaggccttgcaggtccctgccgcaggccttggcttcgcgcctctctcgctcgccccccacgaagatgattgccggtttcaaaccgggagcagggagtctgcttccttctccgctgagtccgaaggatcgcagattggagcgtgctccggagaccgcttttccgcagcgccggcctccgagatccccagcaccccttcagccttaagttcccacgtttcgggtccgtggcgccaattctgctaagtagcaggctaggaattgggggaagtcggagaagaaaccctaagtgtgtcgcccccagcttccgggatgcaggcccgccggggtctagaggggcggctgccgtgcgtccagcctgtgcgcaggcctttcgccgctcggcgccccaggcagcctcagtttcctttcctctgtttgcgccccagtgaacctccgcacctctcattcagggaagagaattccccgcgcagccgcgctcgtttcttcctctgggattttcctgagaatccccaggagttggccacgatcccatggggggtttccttctacccagccccgcgtcctggcctcgtccttaacccccgggttgccttcactcaggctgggaatccacgattgatttcctactacggaagcgggtggcgttcccagcctgctttcggagcagcacgggtttcgtgcagggtgttatcccgaccccttcccccatccctctaatctggcttgagaagcccgtgctggagagaaaaacgcggccttaaaaaaaaaaaaaagtttaaccgaaagcgtgagagccacccgccggctgttatctggggctgaaggctgcggtaatcgatgggttatttttacgcggtaatagggccctgtgattgctctattaacctttagacctgtctgagggactctccggctcgcagccccgctgcgctggggcctccaggctctgacgccgactcccaactcaggcctgacacattcccctcccccataccctggaagagccccctccatgaagaagctcccctggaccgcctggctccccagcccttgccacgtcccttggattggtgcagagccgccgcaggctgcagaaaaaagggggaaagattagaagagaggaggccacaggagatgggaagtgtcgccaggaagggatgcagattgcataaatacataaaattgaggctgaggcctgggctcccgaccatctccctgggattttgggaaggcaaaagggaggcttcggtctctacgctctgattttaggaggcagtctgggtgtctcctgaacctccaaggaatccggggctgggaggatccccactacccctgcccaggaactagcatccagccgggcaccccgggtgacccagtgccccacacaagatcgagagttgagcccaagaggtcaccttcttctctactggccccgcccctcgcccgccgctgcgggatgaggaccacaggaagggggggcggggagggagaaagggaactcattaataaagctgaccctgggcaccacagcgaacccaatcgacctccggctgggttgcgggtgattccccgctccctggcggtagcacttgggcattttccgcggagaccccagagcctggactttgcctgctgggggagctttccgcacagtcccgcagcctgcgcccagcggaggtgtagccggggccgcgcacccccgccccgcccttgcacgtgactcccacaggccagtcagcgccctagggccgagttgctgggccggggacccgagccgcgagctggggacttggaggcggccggcgcaggggccgcgagaggcttcgtcgccgctgcagctccgggggctcccaggggagcgtgcgcggaacctccaggcccagcaggtagggcttttttcttccctttctttgctccttcccgcggtcccccaaactcggagcttctccgcctttgcttgtctggaggtagagaggtagctagtgggaggaaaagagacgtgcgctactcacttcaccgaaattgcccaacccctgctctgcttttgactttgccttagcaacttctttaagtcaaagtaagacttgggggcaaaacagagaaatattggaagcgcctttggattctttccgtgtgaacttgaacgctttcaatccctgtccccgtgtgcacattctccaacccttgtttgcatatcgcaggccggggcctgggtggtgatggtggccgcgtgaagttaccgggactgacgggcccgggacaggctgcacggcagctcgcacatggagggaagtagacggaggcttgtcgcccaccagcgactccggggacgcagggtggcagtgccaggcagctccgctgggcctcaggggcccccgggagccgctctgaggtgcggagaggctgctgagtggcggaactattcatgccctttctggccggcctcctcgccctcggggctggggtccagggactgaatgctcctctggaagctcaccaccccacctgcccgcgctgcttctacctgaaactggccaagggcccgagcccggaccggagccgtgacttccctccgccggccacggggctgcccggatccgccgggttatgtcgcttggctttgggctcaggggtcaccgtgggcagaggggggtgccggggtcgcggactgccaccaggttgaggaaaggaggggccttttggctggggaaagagcgtggtgggggacccgcggccgatggaatccctggggcagcgcggcccgcaccgtggaggttggggaagcgcctcggggaagtgtttcctgtgttcccagaaaaggaagacaaccgagagcaggtttcaggcttttaaagaaagcctggggtgtggaggtgatgctccgcacacgtctgtgtctcctcccctgctgcggccggcttggttgtgccggctagcgtgcgaccgtcctcctcgctgcaggccgagagcggaggcgtaaacccaggccagcgaggagtgtcctataaagggacggggacttttcggcg

SEQ ID NO: 219 - DNA, 357 bp, Homo sapiens
  source 1..357: mol_type = genomic DNA
cgtttgctccgccgtcagtccataaccaggcgctactaaggcctccaggctcggtgcgcagagccgggagcgctcctccagctccaggttgggggagtcggggtgggaggaggttcgggaaatcgactgatttagaggctgggggtgagtagcctcgatttatcatccctgatggtgagagtggaaaagtgtcgcgagagtgacagatggaatctttagtgcttcgcaagagggagttattaggccgcggggtttggggggtactttccgcttactttgtcattctccacaaagtccacgaaggccgtccgctcgatctccaccggctggccctgcctgtcatagagcgccaggacg

SEQ ID NO: 220 - DNA, 2355 bp, Homo sapiens
  source 1..2355: mol_type = genomic DNA
cgctcggtatgttcatctaaacgaccttgggcaagtacgtcgattccaaggtacaatcagccctcccagacggcttttcgagtctccctaaccccggtgggagaggacgcggcgccagagcccagctccggctagttttcccgggggcaggtgtagccttgggcgcggggccgggggaggggcaaggggcgggcgtggggttggactgggacgtgggactcggaccacggcctgggcgtgggcctaacgacgcgggaccggcccgccctcgccgctccattggccacatctgtgcagaaaaggccccgcggcccaggggcgcccgcagtgtcactaggccggctgggggccctgggtacgctgtagaccagaccgcgacaggccagaacacgggcggcggcttcgggccgggagacccgcgcagccctcggggcatctcagtgcctcactccccaccccctcccccgggtcgggggaggcggcgcgtccggcggagggttgaggggagcggggcaggcctggagcgccatgagcagcccggatgcgggatacgccagtgacgaccagagccagacccagagcgcgctgcccgcggtgatggccgggctgggcccctgcccctgggccgagtcgctgagccccatcggggacatgaaggtgaagggcgaggcgccggcgaacagcggagcaccggccggggccgcgggccgagccaagggcgagtcccgtatccggcggccgatgaacgctttcatggtgtgggctaaggacgagcgcaagcggctggcgcagcagaatccagacctgcacaacgccgagttgagcaagatgctgggtgagtccgagtcgcagacccaggcggccgggcgcgctggcgcgaatcgctaggccgatttcttaaaccccaaactgttctttgcgagcctgacgcccaaaaccaggggtgtgtagcggccacgtcctttcttaaggctctgggttcccttcccgcttcccgccctccgaccctccaaagcagctttccgccttgctctccggctcccggattccccaggtggccgggggcgcgggtccaacggctctgggaaggcgacttcccggcacctccgggcggcgcgagagcacccttggccctgaactgggccggttgtgtccatccctcgaccccttccctagttaggtgtccttttctgtttttcgaacgaccgggtgatgggtgagcggaaagccgcttccaggagaccaaaagaaaggggtgcctttagaggacgggtgttccccaagggctcggactcaggagtcccagatctccctctttaacttcaccccggttgcgcaattcaaagtctgaggggggaggtgcgtccaggtggggccaggtggggcctggagcgggagcgcagccgataagccctgcgcccctctcccccttccttccactgtgcaggcaagtcgtggaaggcgctgacgctggcggagaagcggcccttcgtggaggaggcagagcggctgcgcgtgcagcacatgcaggaccaccccaactacaagtaccggccgcggcggcgcaagcaggtgaagcggctgaagcgggtggagggcggcttcctgcacggcctggctgagccgcaggcggccgcgctgggccccgagggcggccgcgtggccatggacggcctgggcctccagttccccgagcagggcttccccgccggcccgccgctgctgcctccgcacatgggcggccactaccgcgactgccagagtctgggcgcgcctccgctcgacggctacccgttgcccacgcccgacacgtccccgctggacggcgtggaccccgacccggctttcttcgccgccccgatgcccggggactgcccggcggccggcacctacagctacgcgcaggtctcggactacgctggccccccggagcctcccgccggtcccatgcacccccgactcggcccagagcccgcgggtccctcgattccgggcctcctggcgccacccagcgcccttcacgtgtactacggcgcgatgggctcgcccggggcgggcggcgggcgcggcttccagatgcagccgcaacaccagcaccagcaccagcaccagcaccaccccccgggccccggacagccgtcgccccctccggaggcactgccctgccgggacggcacggaccccagtcagcccgccgagctcctcggggaggtggaccgcacggaatttgaacagtatctgcacttcgtgtgcaagcctgagatgggcctcccctaccaggggcatgactccggtgtgaatctccccgacagccacggggccatttcctcggtggtgtccgacgccagctccgcg

SEQ ID NO: 221 - DNA, 881 bp, Homo sapiens
  source 1..881: mol_type = genomic DNA
cggggcaagggcgcgttccctatcgcaggatcacttgctatggtaagccgcccaccctgcgcgctcctccgcgcggggaagaacctgcgcggcaggacgtggtgttggagttggggcgcccggagcgtggagtggggagacctgatgcagagagtctggagtcggagctgggggtgctgcaggtaggagcaggagcggggcggagagggaggcccgaagaagaccccacacaggttggcgcagcggggcttggggaggcttcagcccagaagtggagagggttgacagacgcctgcctgattagaaaaagccgggagcttgggaaggagacgggattgaagaagccacccggcagggaggccgaacggcccagagctctccgggtaaaacccgcctgcggtgatctgggaagtgtgtctccacctagccctgcgagcagcggccttcctcccgcccgttagaagggcgctgtgctggagtacgaacccggcccagagaagccactcgcccttctttgtcacttaaaaccctgtcccgacgcggatctcacgtctagacctctgtctttaaagcggatgtagagcgcgttctaaccgttccctaaccattgtgtcaccgcgaaaggccggggctgtgtggaaccgtcccgcacgtgtgcgatgaatctggctccgctgagaacggatccgtgggctgtctggcgcgggctggggcagcagccgagagttagtctacagagctagggcccgagggtggacctgcgtccgcgtctcgtcacgaaaggaagctctttttggagaggcaaaacgtggtcgcccagatccggcgcagctgtagccgtgggcgctgtgcagtgacagccacacccgccacctgtcacg

SEQ ID NO: 222 - DNA, 236 bp, Homo sapiens
  source 1..236: mol_type = genomic DNA
cggccgccccgatgcacagcgtccccatgcaggagaagaagcccacagtcaccatgttctccatagacacgtcagtgttgcgcatgccacagcacttcttaatgcgcttcagcaggtagcgcacgaaggtgttcatgcgctcgcccaggctctggaacatgaccagtgtcagcgggatgcccagcacggcgtagaacatgcagaaggccttgcccgcatcggtgccaggtgcagcg

SEQ ID NO: 223 - DNA, 288 bp, Homo sapiens
  source 1..288: mol_type = genomic DNA
cgctgaccacctgccgcagggccgcagccctcccagaccgacagagtgccaccagctcccgccgcttgtcagcgccaacgtattcggagagcagcaggtcccagagtgacacgctacaccccttaaacctccctgtgctgacgggcaccttcatggccctcagagccaccgcggtgtggtcgtccacctcaagcactgtgtctgagggcagtggcaacagcaacaatccggtctcggagtcgcacacgcagcgctccctgagctgctggtaggtcacctggtcccgcg

SEQ ID NO: 224 - DNA, 3747 bp, Homo sapiens
  source 1..3747: mol_type = genomic DNA
cgtgtgcccttcggcgggcggctgtagctgtagctgttgtgacggctacggcggaggctgcggccgcgcggggaatggagccggaccgcggagtcgtcacctccaaggtgtttctagtggcctcctggaagatgatcccgcgcccaccttgccggcgtgttcgcgggcccctgcccactgccccccctctttctttaggtctggcttttgaggatcccggagtcttgcagctcccgccattccgcagataacctccgcacacttaattgtgacccgcggggtggtcggagaagctccgcacgcgtcctcagtgggaaagtgtcccctctcagcactgcccccttcagtccccctgcatttctggaaagtcaggcaaggcccaggtaggctgccagtgcactctcagccttgactgagcacctttcgggcctctggtgagccccgagagccaggtttggctcggcagagcgggcttgggctgcgcaagcaatgcgcatcgtggccgcctgcaccctgggaactaggcctgtccagtgggcagcatcctcatttttgaaaaggcccttctaaacccaccggcctcgcctaacgccgtttggtgctgcatccgagcggcctcacgcggtcccctggaagggccactcccagcgggagggcggggcccgggacctgcgctggccacgcagggtctcaagctgaccggaagacccggcttttggcctgtgtccaatagccccagaagagaggggtctggagccttcctcccacgcgtagtgacgctcaggtgtcctcgggttgttagtcttgacccaggagagtgcaagacaggcccaaggcctggggggattgcgttatgaatgtccaattctaaacacaaggtagagcacagacagtacatcctcacggctggattttaaccgtattttaataaacacattcgagggggtgtcagtttccccaagctctgcccccttccgcgggcgggatccatggtgtgtgcagtgtaagagtgcgcagaacgcgtgtgttcaagtgtgggcgtggcaggcgtcgtgtgctcgccccgcgcactgtgcggatcgcccagacagccttgacaggtttttgcagatgtttgggtgctacggtgtggggaaacccaggcaggagcgccaggcctaattctcctggactcttggtgagcggccgctactccacgaggggctagaagcaaagggggcacgcgcttttccccaggccgcctcttgctgccgcagtggctgagggcgctgatgacccctccccgcttccagcggacttgacccgcgggctgacaacccaccgcgacaagcaggcggctgggttcgcgccgccgccccggggcccttggctcaaatttcacctcgagtcctgcagaccctgcgccactgaattggggcccaggacgcccttggtgacactcgccttcttgctgccacaaccaccgtcatacccgcagccggggctccctccgctaaccacgcttggagaccccaatcggggacagaggtgggagtcagaccccccctggcctgcactgccgtttccctcgattcttgcggaaacaagactcccgcccacacataaaaatgcagctcccggccaccgggcgccggtggctcacgcctgtaatcccaacaccttgggaggccgaggcgggcggatcacttgaggtcaggagttcgagagcagcctgaccgactactaaaaatacaaaaattagccaggagtagtggtgcatgcctgtaatcccagctactcgggaggctgaggcacaagaatcgtctgaacccgggaagcggagggaagcagcgagtcgagatcgcgccactgcactccagcctgggcgacagaatgagattccgtctcaacaaataaatagaaataaaaatatgcagccccctccgctccacttgaactttaatgctgaaccggtttcccacgtatacgtgtatcgcaccgcattttgacgctttgcatcgagtcgcattaatggcgcttttgagaacgcgtcgtcgcgctttacagagaaaccctacgggcagcctgtggaggggtaggggatattcattggctttccctgctgggccccgtccgccgggcgggttagggtcgtggcagcctgcccgcgcgccgctgactctggaattttgtccgggaaactggcgtagggccctggctctcccttcgccctccgcgcacacgcggacgaggccttagatccacagccttttctaggccctgcgcctttgaagctgggccactgccaaccgctcgcgattctcaccttcaacagtcgcccccttacccctcccccacccgcctgccctcgggagcgggtcgcctccactccaccacctgtttaagttcctccccctcggcgcccctccagtccccaccccggccccggtccaaaaaaccagcaaacggaacttttccacagttgaaagccgcggcccgcgaggccgggctgggaggggaaagcggggcgtgtctggggggcggggccccgagcactccggaagttgccccgcccaggaggctcctgggaaagtgaggagagggcccgggcctactttcgtcctggtgtggcgcccctcagcctcccctcctcccagttcccgcgcctccgcagggcgcctcggcctggcctccaggcaaagttcgcgccccctgttcctggggtgtcggccgcgcgggccgtttcccttcattactcccgggcccctgaatccgaacgctttcccagaagcgcgcaaatccgcttgctttccccgcggctgggctttgttcagggacagcaaaggaggaggcgggaggctggtgaggttttctggaaaaggggcttgtcccgaggaggaagtgccccagatccctgagagccaacgctctggggagaagaaactttccttctcccttgaatgttgctcagattacctaaaattattttttcagcccttgtgttctaaagtcgcagggtaaaggttatcttaagacttaacatcagcgctgctcatttgtacgttggtggagacgtgccttttttcctttgcacttaaggtggacagggtctgcgacgctcccttccaggacggtgtggggaagcggccgacgtccccagccggactcacgccctcctactactgggcgtcggctccgccgcgggcgctcccgacagggagctggagtcggacgagcggctgcccccagggcctccaggaaccgcggcccagcggggagcgccccaggctagcgcttttccagttcccttcgaaagcgcggggctgaggtcgcggcgctgggccctcggatgaagccgtgctgtagctacacctgaaccccgcgaaaggctggcgcggtcgtgtatccaggctgggtctgaggaatccgcaagcgggagagcgctaactcctaggcgtgagccgctgctggcttcgagagttcgagaacatgaaggacctggcttctcccgcccggtcggcttagggccagcgaggtcacaggccgttctgctctccctgtttgtccccaaaggcctcggcacgtggggatctggagcaggcctcaggctgcgacccgtctcttcccctaccaaaattatgtgggaacagcggtccaggaccttcccctgttcagcggtatccccgggccggtgaccccggggttcagtcgtctccccgaccccaagcggcctctgctttccacccctcgccccggagggcggcttcg

SEQ ID NO: 225 - DNA, 335 bp, Homo sapiens
  source 1..335: mol_type = genomic DNA
cgcggccagtgaaatcccaatcgtcttccacgtggaaccccaggtccgcagttatgataacggatcacatcgctcctgcggaaagtgcgcgcggtggagtgataattggacctagcgtctaaattcttgttggaggacctcgttccagctgccagttaagcctctgggatccgcagcgtctctaggaattgagagagtggggaagttaggatccaggaggaggatggtgggggctgaggagtggaggagcagcgtgcatctcatctcttgtcgccgggcgggcgctctttcgggtccagggcccttgcacccccagcgtggctccggaggcggcg

SEQ ID NO: 226 - DNA, 255 bp, Homo sapiens
  source 1..255: mol_type = genomic DNA
cgactatgcaaacgcttgccgctagccgcatccttctatcgccccccaactcaagacccgaaatggccaactcttcaccgagggggcgtcccatcgctgccttgccttggcccgccccttcgccaagctcaatcccttttccccagaaaaacctgtgcctgtcctcagacccggccccacggctggaagtcaaaacccaccccggagccacgcggctggagcacgtttttacacctgctcggccgcttaattacg

SEQ ID NO: 227 - DNA, 4206 bp, Homo sapiens
  source 1..4206: mol_type = genomic DNA
cgctcacaggtcggaataattcaagccttccgctcccccgccgagctggggtagctgatcactgagctgaaactaaacgttttaggtggaaaaaaagcgtccgaaggcaccgtgaaatgattaaggaactaaagagcttctcgccatgtgagatcatgtcctgttctcgccaacatcacaagatgtccccagacacgccgcgcccccagcgcgccgccccacactgccggcccggagcgaggaaagggtaggcgctgcgcggccgggcctgctcagcgcgccagacgtggcggacccggcccggccggagtagagcgggaagccgggagagcagcagtgctgctgccgcgccgccccagacttttataggggttggggggagggaaggaaggcttcagcctgcgccgggcgctagccagcgcacctacgggaaggggcagaccgaagcggagcccgggagcccgggtcttccagccgcaccttctattaagacatgcgcttgggggtcgcggccctctgcgctgtgcgcggggcctgtgacatctccatgtgcaaaccccgctgcttagacgagcgcaggccgggtttttgtttttgaagaaattaactgctcggccaagcaggtgcttttgaaaggggttgcaaaggctggcagtgttgccaggcaccgaggcgacgttccggtttactccgattcacccctagggcggcgcccgaccccagggttcccagccatccaacccccaccccaatttgttggggtcctgactattgctgggcccctgaccctagggcagcgccagatccctaaagcccccaggtcccatccagtcttctagtcccagaacccctccccacatcaagttccattctggcccagagcatggaggttaagctgtgaaatactggagatgaaatccgggcttccgctttggagcacccgggcagagcagaacatctcagtagtttctggagattggcgggagttccagacaccttaccccggcccataagtggaatcgcattgccctcgagccctgagacgttgtggagtcccagagccccaggacttgattccagagaggaaatcagatccacagagaccgagcagaggtgtggcatgtccagccctctctcttcgggccagactcaagcctcccctccctgttcccctccccagccttgagttagttcagctgctgggcctagccactgaaagcctgtggggccctgcagcggggtggtcgcaggggaaaggttctccagggcgggggagcgctccgtaggaggactcctggcccgccggacaatggcagttttggagagtgaatcgtcctcagcaagagtgttgtttgttttttggagggggtggtcacgaggctgggtcgcaacctagggggaagagcctagagctcgaggaaaggggggcccccggcggcaactggacatcacatatccactgagcagatttcgccacaatcggaaatgtggctgtggattcccgggcggcccccccaccaagtggtctgcgcagttccccagcgccgcctcctccctgcagcctgcgggcccgtgtccctaccccgagatttgatttttggccatctccgagtgctcgactctttccctccgcagcccagccccagcccaggcttcccgccctcccgcctcgcccgcgccgggaggcagcgcctgggaggagcggcctgtccgcgcctggagatcaaaagccctcccggaattgacatttcaccccgggctgggggcggggggtgttggtgttttgttgttgttgttgtttccatttagagggcttgttttgtttttgtttgttttctgtgagttttcatttttccatgggatattcttttgtttcgggtgttttgcgtgttgttttttcgtgggattgattttagcgggttttgctctgtaagtcctggtctttggggtgagttttgattttgtgtaattttgtactttgtccttgtaggctgtctttttgtgtgtcgtttgtaaggcttttccttctccgcggtgtttagatgtgttttgatttggtgtgagtgggtgtcgtctgtgtgtgtgttgccttagggaatttcgtgttgtgtgtgttttgtttccttcgatttgaattttatttttttgaactgtacgttttggtgggggatcgggttatattttcatgtgttgtctttagtttttgtgtttgtgagatgcgctggtgagagtttgcgtgtgtattttggcttttgtggggctttgggattagggaggggacatcagccgctactccgttcctgcccgcacccatgaagggcagaggattaggccaagcatcacgaatcgtaggttcgccgtgctccgccgcttcgcgctctggacgctcgagaccagcgggaaacaaatgtggttcccgagggggcgcccgctggtcgtcaaaacctcgcgggcccggaggcgccgctgctgggtcagaatcgcagagcccccctcgcggggtaccgaggcccagccgccgtccccttgcccaccctctcccctgtgagtcggtctccccttgcgcctggccagcatgcttcggaccgtccggactcccgcagcgttggcccaaggggcggctcccctcggcggagcaggtgacgaaccacttcggaggacacggaattgacactgcggccctagagatacgggacaaggggccagaacgcggggagtctaggctcagatcggagtcgggatcccgggaaaatcccccatctcccttgcccgcagccctagaatcccagaaacccaactccgcgcagctttcggtttggagagcccgccctggtttgcaagcacagatacatgtgcccacacccgaacacacgcagacaccgccagagagcgagacagcgagcgggcgcctccgtagcagcggcctgcggggcggaaccggagctggcgccgcctcaaggggaaaccgaactgtagtgaccggaaatgtccccaggacagaataagccattccttctgtccacgttgccaggacttctccagctcaacacaccttggtttgtttaatgttacacgagggagaagatttagaggcagagacacatgctggctcaatcggcgaggaaatacgagcagagtctaaacagaaagaaatatggaacggggggagactagactgtggggaaggcaaattggaggcccagaaaaggggctggtgcggaagctcctccaggcccctgggcgctctggcattctgtgagaaggagaaagcagaagccgagaacagagagtgacaggaaaagaaaagagacgcagaaggggttggcctaggcacgctgcgagctgccctccagctaagacgctgcgggcaagggagggggaggccgacatcgccaggcgaggggccaaggtcgggctctttaactcagccaggaaacatcgttgggggctccagggctcgtggagcccgaactggggctcatttccggcccgatgcccctcttccccagacccctagcccggaatccagcaaaggctgggccatcgccgtccgccactgggcctccctctgtgcgagccctgggagaagcctcagggaacgactctggcactggggctcggccccggagctagcgcagagcgtgggtgggagtcgacatccaggcaggaggcaaagaagaggccccgccctaaagccgagggcccagttcttcgaaagcctgtggccggagctggactcacaggattcggggtccgtacagcgcgcacgtggtaccgcatcttcggcaggcggcgcccctaatgcccagccccgccctgcttcgtttgccgggcctgaactgtgtcagcattgagggtggacgccgagtcgcggcgagttggtcccttgcggtataaacttggggcttccctaggggcggtgccgggatggatgggggcgcctctccacctgcaactcccccagcgcagagcgcaaaaagtgcacatcgcgggccagccgtcgcctcggggtttcaggcggcgcccggggcaaaaaggagctgaggattggtttgggtcaaaacacccatttcagttggcagtggggtcctctgcgcctacagggcttcgagctccgggtgcgggctcactggggcaccatgtgccagcgactcgccacccggaaatacttgggttgcgctagaatacgcaatgggtctgtgttcg

SEQ ID NO: 228 - DNA, 5770 bp, Homo sapiens
  source 1..5770: mol_type = genomic DNA
cgggccgccaccgccgcctcggctcctccgtgacgtccgggaagggtatccccggcactcaggaagtggtggccgcgggcagctccgcccccacgcgactcggggctggccgagctgccgggtggtagaaccgccgggccttccgcagggcgggcggggatcaccgtctccccacctccgtgcgcacacacatgcacgggcctcgcgccccgctgtcgcccgcgcctcgccctgctcacttctcacagcccgcgccggacgaagagagccgccagcgccccccctctctcccacggctcgcacccactcaccccgacccccggcgccgagcccggcccctcgtcgccggtcaccagacctggtcgaaccgagtccaagatggcgactccgcccccgccccctccgcactcccggcggggtcagggcgccccctcacgcggcgggctcgcgcccgagacccggctaaaaatagccaggcccgactatatttggttcggccggatcctggcgcggccgcgcgctccccgccccgccccgccccgccccgcgcgccctgccccgccccgcgcggcgttgaatggagaaggggcgggggtgaccgaggggaacctactccgctatctgcggcgcgcgccggcggcgggtcccggccaaccgccgaatttagtaacatcgcctgcgtcaatcacgcgcctcgcgtgcgtcagcgccgcgcggctccaggtcctgctcccccccttcaagcctttgaatggatacaatgtagcagcgccctccttccttccgaggctggattggaacccgccgcagtgcagagactcggttgctctcggctgggtcaactttcggggcattctcccacgatcctctccgcaccaccgtgtctgaattggaagtggaggcgaagaaagatatacatgccatatttacctatatgtagtttgttttcaagtttctggtcctagctcgaaccttcttcgattctgaaatgtgtgctgtctacaaaggaatcttgtatctcccctcggcgcagcgccccccgcccccgccacacacacacaaattgggacaggtcaaacatataaaacggtatttgtgattcaagcggaccacatggggaccactctatctgcattgtttcactcaaatattttctcctgtccaaaaattcatttctgaaagagactgcgttcactcagcagcaacctttgggactaggggtctttaactctgataaattttgttttcatcaagaaatttacacttaaatttatcatttccaggaagaaattgctctccttcatacagtcacccaggctttcggcacaccatttcatgacaaatgtgtccgaggagaccaaagcaaatcccctagcgagggactgactaataagtcctgttgattgatttcgaaatgtttaatttgggagatgtgggcggagggcatctacaaccatcaaaaagtgaaagtgctagttgagagttccatttctgacccggtgccggggaggaggaatgatttgcaatagtcagacccgctcagctgttcaacacgtgtgtgtttgttttacacacagagtagtttctgctgcagcgcgcgtgtgcatgatggatgtgcacttcgctgggttataacgtgtccagttaagaaacccacgccgtacgtgtaaagaaatcaaaccttatccccggaaccatctgcatccctgtgtgaaacacgcacccagtaaatgatgcggggaggggggattagcctgggcgcagaggaccggagcaacgtaaacagctttagaacctatgcaagaggaaagtgcagctgcacctcagggcgtcttcgggctggtgccagacgccttctgcaccggctgccaggtcactggagctggtcagaagctggctggcggagccttccctttcggaagagctgtcctctcccttacccccctcgccctggctccgtgcctcggggcagcctcggaggcgcgccagcagcactcctccaactctactccacccgagcctgacagctgggcggtcccgcctgacccgtgggcaggccgctgcaccctcccgcagacgcacgccctggcgagcggttccgctgcaaaaagagaagcccccaggccggggccggccgtgcggcggagtttccattgtgcggccgtgcgactggccgaggaacgcgcgcgcgcgcgcgcgcacacgaacacacacaccctccctcgcacacgcggaaccggctgggccaggggagggaggaggagggtgacgtagcgtcccatggcgtcacattgacgtctcgcattccaggcactctatggagaggccgctagggctcctgtggcataaatgacgtgccgagagagcgagcgaacgcgcagccgggagagcggagtctcctgcctcccgccccccacccctccagctcctgctcctcctccgctccccatacacagacgcgctcacacccgctccctcactcgcacacacagacacaagcgcgcacacaggctccgcacacacacttcgctctcccgcgcgctcacacccctcttgccctgagcccttgccggtgcagcgcggcgccgcagctggacgcccctcccgggctcactttgcaacgctgacggtgccggcagtggccgtggaggtgggaacagcggcggcatcctcccccctggtcacagcccaagccaggacgcccgcggaacctctcggctgtgctctcccatgagtcgggatcgcagcatcccccaccagccgctcaccgcctccgggagccgctgggcttgtacaccgcagcccttccgggacagcagctgtgactcccccccagtgcagatttcgggacagctctctagaaactcgctctaaagacggaaccgccacagcactcaagtacgtatgagattcgcccagttaataaggggacttgctaaaaaagttggcttgctgggaagcgtcgctcctgaaattgacaactttgagtgtggggttccctcccctgaccttgccgcccggctccggctttgctagcccagcggcccgtaggttctgatctgaaacttttcccctgtagtgggcccgcggggatctctggagctcgtgggattcctccccccactcgaagaggcgaaaagcctctaacaaagaggaggaccgggatttgtgctatagcggctccagcgatgtaattcagggtatttcggctctagttgtcatggtaatgatgctctcggcggcggcggcggcggcggcagcggcagcggcagggagttgcagctccggtgatgaacggcagtaattttcctgccttttaagtaggattgaaaataggagctctggtgggtccaagtaaatgttgctaatggtggcaccgagctggttctctggaaggaagcttaggagggaagggcctaggcagcgacggccagagtttgcagacgcactcggaggacctcaggaaagagggcgtaattgtaggaatgtggcttattgtattgaaatgagcctggggttccactaggcacgtctgcctgtcgtcgctgacatgccggtttacacttttcccttaggaggtaaatcgggtgtaattggcgactcccgcacactgacacgtgtggggacggtgtccctctcctctggacgttggctcggtgtgggaaaggcatgctttttcacggacagaactcgcgcttttggagaagttgctccgagtgttttactcttagtagaaatgaaagttctcggtggtgagacgagagcggtcatgcgagcgcagcctgcggagactgacttccaaaggcacctctccaacctgttggaagcctcggggcagtggtgaggaagaacgcatttgtcccttgatttccaaaaacgttcttggagattgccttttatttatttatgcgtccggattctccagagggaagagctgcgaggagttagtaatcagaaatgtcgggctgtgatcccagggccttgtggtgctgggagagaagaacagggagccgtcattccatcgcgagtccagtgccggatctccttttcccacgaccgggaaatattttatagaatcaagttagggaaagaaaaagctgcctaacgtggctgatgggcacaatgaatgaagttactttattcgagttattttcgctgaacaagatgctcgcagagttagtatcaaagcctccttaggccacagcaacctccacgtccgctccgccccccaccccccggcacccatccacccctttgcctgggaccgtgcgctcacagaaattcttccccgcggtttgtctagtctccctcggtctttctgagaatctcagtctttccctttctgtcagtctctccctcgctcctcttgcccccgcccaacctccgcccccgctccccagggttggaaactccgcagaggagccccggtcacaatggtgcgtttcacggtttcctccacacacttcaccaccgggaggggaacacgggggtgttcctttcgtctgcccacgagaaagaaaggggctttgacagaggtagttatttcttcctaatttacaaccccaaggctctgtgctcccggccccggcggccgcaagcaggagcggcggaacgtgtttccaggcactgaggctttgcctaggtaaccggccgcttgtggggtggagagagcggcggggcgctgggctgggctggaggaaaggctgtgtgggtccctcccgcgaacgctgggcgctcgaggggaactccttcgttggggagaggcggtccccgcccacccgagttctgaggcctcctctcagagaacgactccgcttgaaaggccctcagggaatgcgggacgggaatgcgggcacccactgaaggggcgggtgggggattgctggagacccccattgaagatgcggggagacgggaagcacgtgctacagccgtgggaagatccgcttctacccgcgctacatcgagcgcaagctttccgtccgagcagctccagctggggctgggggtggcaagggaatcccccatttcctggtcgtcgagggtttcctggacctgggagccccgagtccctttctctcttcctgcaggcaagccccgggctcaggggcgccaggagatgaccctcgtggccccgcgcacctcggaggtttttggtttggtttgatttggggtttttcggtccctgttcccactcttcgagctgcgcccaccccgggcaaagggggcgctataggccggagtttgggcacccagttccttccctgaggcctgctgagctgcgctttcagctacaaagtttcgctgaggctgtgggctgagacgctggtcccggagctgcgctcggcgccctcaggaatgtgcccccgctaggccgctgggtggccaggagcctccaagccgcccaccctcgaagacaccgccctctgggtgcaggggacctgcctccgctcgtcccatcagccgctaacgccgtccgcttcgtccccttgcttccagcgccagccttctcatcgctgtgcccttttgtttgcttgacccctggccctcgaaactcgcggctaatagaagcgaagctccattagcatttagaatgaaaagcgcagactttcttaattcctcggggcattcatgcattcgttccggaccttgtgcatttcctatgcaacgtgtaaaatttgtatttgaggggtgggggcgggcggagctggaaaatggcttttttccgctggtgaacactcactgaccccccgtattcgggcggagctcg

SEQ ID NO: 229 - DNA, 582 bp, Homo sapiens
  source 1..582: mol_type = genomic DNA
cgctgttagatttcgggtcaatatccatcatcgctgttagactcggggccgacatccgtcatcgctgttagactcggggtcgacacccatcatcgccgttggactcggggctgatacccgtcatcgctgttagatgcggggtcgatatccatcatcgctgttaaatttggggtcgatatccgtcatgactgttagatttggggtcgatattcctcatgactgttagactcggggctgatatctgtcatgactgttagactcggggccgatatctgtcatcactgttagactcgggtcaatacccgtcatcactgttacactcggggccgatacccgtcaccgctgttggactcgggtcgatacccgtcaccgctgttggactcgggttgatacccgtcaccgctgttggactcgggtcgatacccgtcaccgctgttggactcgggtcgatacccgtcatcgctgttggactcgggtcgatacccgtcatcgctgttggactccggtcgatatccgtcatcagtgttgggctcaggtcgatacccgtcatcagtgttggactcgggtcgatacccatcatcg

SEQ ID NO: 230 - DNA, 254 bp, Homo sapiens
  source 1..254: mol_type = genomic DNA
cgttagagcatagcgacatcacgtgcggtttcttaatgtccctggtggcggatacgccgagtcctcggaaggacatctggacaccactttcagccacctccttgcaggggcgacatccgccaaagtcatcctttattccgagtaataactttaattcctttctaacatttacacggcaaacaggaatgcagtaaacgtccacgtccgtcccacggctgggctgccgttccgtttcctccacgaacgggtacgcg

SEQ ID NO: 231 - DNA, 14472 bp, Homo sapiens
  source 1..14472: mol_type = genomic DNA
cgctcccaaagccacgcgaaagtggaagcgcggggcctcgggatgcccatccagccggctgcccgcgcgctggccggggttccaggactccttcctgctccgccgggctctgcagcgccgcctggtggacacgcggcgctccgcccgggctccagccgccagccggcgcgccggggatcccgcggctgttcgggaaaaagctggagagagtcggggggcgcgccttggggaggggcatttgtccctccgaggaggctcgcacctcggcctggacacgcagcagatactggcacccacccttcacttaccgcgcgcccggggtgactcggatccgtccaacacgtcggggaatcctttctgtcctcacccccgggcgccccagcgccggaacgctgctgcctctgtgtagctgctcccggaaggagtttcatcaaacttttaaggggctttggttttgggttgtgttgataaaataccaagaaggggcatgaaggcacgaacgtcccgggttcgtctccctgctggtcccaagcctgaatcccagggcgggggaatgtctaggtccttccgggccggcaaggtgtggtcgctcagggacccctgtccgaagacgctagggaaaagaggcacagcctgtgggtcgcagtggccaggagcgcgtggccgctgctggtgagagagtggtagacgcctggctttcaggtcctgagctgcgggcaccggagcgtgggaccccggctgcagcaccgccacgcgctcccgccctcccacctgcagggcgggggatgtctgtccaagaggccggggcgacaagcccgccggccaggattctcaaggaaccaggcccagctcagcctctctcggcgggaccagagtgggaccggggccgcggcgtccgaagacgctgcgggccaggggtcctcctcggcgccagctccgtttcctggggtctcgcgacgtccggacatcagggtcgggggtgtggagacggcggcggagccagagtccccaccaagtcagttccaggaggcggcccgcgcgcttcccgcagtgtccgggaggtcgctgggggtggctttgcgtgcaacccgggtaaaggccctgcagccgtgaggctggcgctgggaggagggtggaaaatctcaaagtcaccaatcccggtgcaaatggcggcaggggccgcgggctgtcggacgcaggcgagaggccaaaggctgacttcgcgcggccgtgagtccccagaggcaacaggggtacctgagcgccgaggggatcccgagactcggagaaaccggaagagcctgaccccagggagcggagagtttggggtgcgctctcagagctgtgactccacggtcccggaatccttggaaagggcgctctgggctcagagctcccaactagccggaggacctgggctagcgcccaggcctggagcgtctgggagggggcgctgggtctcggcccccctccccccaaaagggactgagacttttttctgcgtgcttccttcggcgcttcgggcagctctgtcctgcggcccaagctggggagaagacagcggcccgcgccacagggagctgcgcccggacccagactcccgccgcgcttctgcagagcggagccctaggtgcccacctggtagccccagaaaggccggacctgggcgccgggacgctcgcggggccgcacttggaggggctttccgggtcctggccgggcgggctctcctgcggcgcggaatggaatagagcgccggctgcagagccacccggacggggaaaagcagcggtggcgccggccagccccgggtcccgactctggagggaggaaggagcgggcgggtgggggtgggggtgagggcgagggttgcggggagcgtttagaaggccctgggcagccagaagaagaaaagaggacgcactctcccctagggaccagagggtccctgcgtactccccccaggcccgggacacaggttcccccagcgcccctccgcctcccagtctattcggcttgccccagcgcgctgccccacgtccccgaggccccggcccaggcccagccggcgagtcccggcctgctccactctgcacaaaacgaaacccaaatgcccaaaaagctcaggagggaaatttaacaaaaaccctgtcccccgccccacaccccctttcacttttaacaagccagctgccaagagaaaattgaaataaaaacgaaatgatagatagcggaggacactattttccaaatggtgaaatatcctctaaaaatatgttccccaaggccaacttcgcggctggtagccccttccgacgcctttgcctcccagaaaatcacaacaaagcgatcggaaattcagccacggtcccgggaagaaggagtagcagtgaggccccggaacccactgcggccgaaactgccatgctctctttaaccaaaataaaaaagataagaagaagaagtaaaaccctttaatacatcaaatatacggaattttaatctttaaagcgatacattgtctattattttagtacatgacgtaaaccttgtccccttctcagcgggtggacttaaaaattaaaaatagttaagtgttccttttaaagaacaaaataaggcaaatgaggttttggaatagaattttttctttttctttttttttttgttgttttcttccagaatacatacaaaaaaatacccattctcttcgatggtatacaccttaaaaataattgcaatttgaaatcagagctgacaaattgtgactttttttttcattttttttgtaacaaacatgcatgtaaatttgtgtttcaatcagacattaaataacgtacaatacaatcatagcaattttaaagtaacattaaagagaagacctctagttctgtggcggttttgcttttatttataatctacaagggatgggagggtttgttttccacatttttaccctcaagttttaaaatttttcccacctcctttttacctacagagctcaaactaaataatgcacttttgaaccaccggtccgcttggagctaacataaataaataccagtgtccaccgatgcagtcctcagatgaacactttctcaattattttttcctttttttttctataaaaagtcaaatgatattttttcaacttttataaagtttgggtggggaggtgaaagtggggagaaggggagtgtcccaccgggtctcggtcgctgctgtcgggtagatagatacggtatacatttctttcctttcgtggcccgagtcctccccacgcgcgggtgtcagcagcctgggccgtgatcccgcctctccctggactcctcccctccccctctccctcctctccctcgctcgccctcccggccgccctcactgataccccagcgccgaggcggtggtcagtctctgtccgtagagggcgtagggggagtagtacggtccggtggcggcgggcaccggcacgggggcgccaggcgtgggaagcgggctcttggagtaggggtggtagcggctgctgagtcccagcgcgtggtgggggctgcgcagcgccagcgtcccagggctgcccggtgcgcccgaggtggggatgtgcatgtggcaagccatggcggccgcggcagcgctggccagagacgacgagctggggtagcccgacagcagtttgtctgtcccgggaaatgccgtatgggtccgcaagtggctcagcagctcttcggacgtggcgaagcgcttgtcgcacggcccgttggccgacacccagttgcagatgtgggggagtgggtcgttagggagcataaagccgtaggggtagagggggtggccggccagggagggcggtgtggcgccagcagccgcggcggccgttagcgaggagtgcacaccgtgcagcgggtgcgtggggtacaccagcgggtatccggacttcagcgccgcagccgcagcagccggatcatgtgcgcaagaagcgctggcggccgccgcccctgccaggtggctagcgcagtggtagctgaggcagtaagggtcccggcacaaactggctgtcatcacggacggcggagacgctccggccaaagggctggagccggccggcttactgcagcccagagacccggccgcggccgccgccagctgcgcccccaccaggctgcccggcttggtggggtcaagtgccacgccgtgtggcaggaactggggcgggtagccggcgtaggccccggccaggctgcctgggtaggtcatacccgcgggaggcagagggaacactgtctggcccggcttgtagggtgacacgggagccaccagcccagagcccaacactgaggaggaggtgggcgcgctggggccggagccggagctggagcccgatgaaccgccgcagtccgagcccagagccttgcctcccgggcccccatccggatgctggttcacatccacattaatcccgccgccgcagctaatccggccgtgtgccagccccgtgggtcccccttcggccgaggcgcccccggtgcccttgccaccgccgcccacgtcggtgtctttcttgtcgtccttgccctccggggcacccccggccgaggacagcatacctcccggcgagcaggccgaggcgctggagctcgggctgcctgtcctgggcgtgaatggctggcaggtggcgctcggtacccggaatcccgacttctccgacgaaacacccccgccgccgcccccgccaccgccaccgcctccaccgccgcctcccggctccttcttatccgagccgggtttggagtacggcttgaaactcgacttgtcctccacgccgatgtcgctcagcttcagggggcccgatttggtgtccttgtcgcccgcagcaccgccgccggcaccgcccgcgccgcccccgttggaggcaaccgaggagagtttggaggagggcgaggggtcgggcttcccgatctgcgaacatgtttgcgccaacagcgccagcgggctcttcttggcatcgagctgcggggtcagacgatggggggggagcgtcacacagagaaagaagtggaaaccctttagaatcctggcttctggggttcaaatgcctctccgcaacagcccacgaagatcctcccagccccaaggcgcaattctaggcggcaccccctcttcaacacccactcacaaacattctcgccgttgggaaaggcagcagattgctccccccgcccaaccaccacccccagcagcatctttcctgccctctctgagcgctaactagatttttaaagagcaaaatgttttggttttcggttcctagacccagaactgtaccccctccccacaaacactccttaattcttcagagtaagggctcgggcggctggcgcctcagaggctgtgtgcacacactggcttctgcggatacgaaggacggcgactttggaaccgtatttcagacctccgcctgccttcggtgccgagtgaaggggcctgggtcggggtaggggctttcatatcgaaaggagaaacaagtattggcagccttgctccctagcatgcacgccccattccacttcctcccccttttccgccctagttttgaggtacggaagcagtagcctcttcccccattcgggagctgaatcactccgacccccccacctccgcccagatcccgagaaaaagaaagccctagggtggcagccagggtagtggtcccagtgcgatccagagagagggtccttacctcgatggggctgaccggcgtggaaggcaggggctgcaggtactcggggtgcaaaatgtggccagttcgtgccgtcagcatcttcagcaccttgattggcaggcggttggcctggcgcagggggtcagaggggggcacggcgtgcacaaaaggcttggtgctgccggccggggacgagcctgggccggggccggagctatttccagagagcgcgctggtccaggcagggtctgcaccgccgcctccgcctccgccgccgccgccgccgctgtgcttactgcttcttagggcagaaagcgagggcgctgtgctcatgacccacccgcgcgcatgggagcagcgggggggagggctccgggaggcgcggggcgggctcggggctgcgcgctcgccccgggagcaggagcagcgggaggaggaggagctggcgcggcggccacgggcgcccagcgcgccttctcggcgcctggagccagacgcgagtaatcctgggtggcccgcagcggagccgtggccgggctagaggagccggctggactgcgggagtgccgggcggctcgcggtgtccgcctcgggctgctcccctgcgctgcgttctcgcggccccgcgccggcgctgcgctctgcgatgcgagccgctgcgtgtccagccggggctctggcgaggaaactcacttcaaaagcagctgcaccaagggggagattaaagcctttgccgccttccaatcaaatgggagcccgaggtgattggagggtcaaggggatgtgacgcgtcccgcgccgccgccgccgccgctgccaggactctcagtgccggttttaatgggcagctccctcttcgccgcccccctgtgtgtcccctccctcgatttcgctgggaggacgagatggacatttattctacgtttgccatccgccgcctccctcttttttcctcctcgtcgttcttcctttccccagctcgaaaataaactgaaaccttcttttgaaggggggtgcgcgtgaggaacagactgcggggggtgtccagaaggagcaccggtgggagtgtggacaccggccggactgtcaactccaggggcgaagggaacctgcacacccagtgttttttccttcgcagtaatcgagatcccgcgcggcgcagcgcagccaccagggtaagaaggcaaggtggggagccggagctggaagaagcccgcccgcccgctctaatttcctcagattccgcggcggagaaaccagaagctagatgggcagtcgcagcggcggcggctcaacaccgcgaggagcgctgggctctccgcccttcccggccacgtgacgcccggggacgcgtagattggggcagcagcgggggtcacatgtttcctctgtttcaccctcagtctgtcccccaaccccccattcttactctcccaccctgttttcctccctcccccccttctttgggcatctccacccctccatcaattgtcaatgttcctcgaccgcaatcaatcagttatttgtcagctcttgtcaatcctcccgtgatttatgtcagcttttgttgctgattacaaggcgggtgcgacttgaagggaaaaagagagagggagagagagacggagaggaaggaggagattgagagggaactggaggaggggaaaagaggagcggcctcctgggatgggggtggggtgggggctctaagaaaaagaatgaaagaggcgcacggtgtcaggaaaatgaatagcgagagtaaagtgcgcaggtgcgcccagggcgccgagaggggcgcgcaggcctggagtgtgcgcctgccctctcggtgtcggagagacgcccttccacctctgggagcctcggtctgttggggtcgcggagttcgggcgcggctccgggtacccgagaccagcggcggcaacttctaacacgggagatttcccgccaccccaccccgccgccgcgagtcctcgcggggcgtgttgcgtgcggaggtcaggctgccaccctctgtagttccctaaccccaaactcggagacttctaagagccacaccaccaaggaacttctagtcctggaggtcaatggtggggcaaacctcgcctcaattctttgaccccctcgggtcgtaagcagggctaagaggctgcgaagaagaggcctggccatgggtgtatgggggaagaaacatctcaggctcactcatgccccctccccgaccttctcccacctgccgccatccccgcaggctggggagccagggtaactcggggctgctctctcgaatttattggaacgccgagtcggaatgagctgcgctaggagagccgagggaaggagcgagagagggagggggcggctgcctgtgggaacgcgggttcttccagggaagcggagcgggactgccgcgttcccctcgatttgcaccgtcactcgggttgtttgggaagaaaaggggaggcgctgtgcgtgccaccgggtacctggacctggatgcccagtgtgtatgcatgcctgtggcgctgcgtgagttatcggggctctgatagcgtccggagcgggttcgagggtctcctccaggaactcgcagaaattagagggggtggggaggaggacacacccccttcctcggaacgacttagaagactttgaatctcccctcctccgcttttccgccccggctcttcttttgttgtcaagtccttttgataaatggtggggctgggagctctgggagtcgggagccagtctgtgcccgcgtggggggcgggggccgagcctggagacccggtttcgaccggcgcgcacctggggctgagccgggtgcgcgcgggggtcgggtctggaactgcccttggtatgctcgccctctcctcttggcccatctcactcccctcccccacctgactcccctccccggcttctttctctgtctcccactccgcacgggtaccgagaactttccggggtgagttttagaatttgctcctaggcattctttttctgcgtttggattttatcccttgattctttgggttggtaaactttatgaaatggtaacttgactgaagctcatttggagagaagaggggagtgaggcggtgtgtgcttggggctggtgtttgtcagtgtgagcgtgtaagagcacacctgcattcaagggggtttgtagtgtccgtgcgacatctgagggaggtgagtcggcgagcgggtggcgaggccccacgctgagggaggcggttgtctggtctccggggagcaagtcccaggtgcgcgtttcggagggcgggacagtcctacgctgtctctgcggggcagcgcgtctgtgaagcactcttatccttcgggtgtgtccatgtgtgcctggttactgagtgcgcggccgtctaggtgtcgacccaaccaatggtagtgctcctaacgccgcgagcccacctttgggccgctatcgccgcggcctccccgcgaggcccgggcactgaaattctggggcctgcgacagggccgggggggacgcagccaagggcgtccctccccagcctccagactcagctcttccccctctcctctaattccagcagcttattacgccggcggctcaggggaggcagcctcagcacctgaagcctgggggggccgtggagtggctcctgcgtcccccggtgtggccacacacgctcgtggggctgcgctgtgctgcgtgggtgcgggtcgtggtcagcgtgcgttcgcttttctcaaaactccactccgagggtccgagcgcatgggggcgcctgggggcctgcgcgcccggggcttttgggaggcgccagcgtccgagcctgcgcacctcgcgcaggaggccaaacccccgaaggccggcgcgggcccgggagtgggggccattaattactctgcgccaggcctaaagcctccaacccccagcagcagttggcgtggatgtcctgcggattttatttgcaaacaatggaaatgatttgttttctctaaaaaaggagtgggcaaggcagatattggaggagggggtgggaggaggagaaggaaggggaaagagctgaggaaaaaagtttgaaaatgccttgaactattcactgtcgagatggctaatcagtattgaggccggagggcaggcgggccgcctttcaccgccgcttccctttcatctcgggctcggcggaggcgctcaattaaaagcctatcagtttgtaagtaaatcaacgcctatcagagttgtcacatcaaacaaaacgattattattgcagcccgcctcccctattaatctccctcgaagataatccgcctgacaggtcaggcccggcgagctggaaagcctggcccagagcacccaaacagtcccggactggcgcgcgcccccgggggcctccccacccctgtcctgccccgcctcgcggtccctgggagcggggagagaaagcgcgcgcggaggcctctgcacccaggtccgccccacctgactgggagcaaggcccagattcgcggggccagtaggggacagtcccgcgcggccctcgccgctgctcggggcaggggagaagcctggcgtgcaggagccgcggcccggcccggggtctcgccacggcctgcgctctcctcggctctcatttattttcttcctcccttctttttgttttctctcccgcgtctttctgggctcccccatctttttccttcatttccttttccttttcccttttttttacttgccagggagtatttcccattctggctctctcttcttggtgtgccgtataaacctttcttgcgcagccacactgcctggagtaggatgttctcagggcccgggttatttctcccgggttgccccaggagtgtgcagttcccagaaactcatggcaaagtcctctataatctctttccgagcaggggctctgagaagctctgcgcctgtttaagggttgggtgggtttggaggggtgaagaggaaaaagagggagagagactggtggacagttgtcctattctggaattagtattttgccttggaatttcatagctgtaacgttgccgacctcgctgttggcgttgcccgggcttttcaaatatgtagataagactcaagggggcatccaggcctcagtagtgactatgtgtgtgtgggtgggtgcctgatacaagtaaatacacccagataactgcctctgtggcctcaagttagtgtcccaagagtttgggtggggcctcgcttctagaaggcgcaaaaatcgtggccttcttcccagtggcctccggcttcctgctttgggatgcctcggaatcgagccggacccgcctttttctgcgctcttcctccacatctgtaggcccaaacggccactgccggagccaggtggagccaggattgaggggtagctgaagtgtctgtgacgaaagggctgcccgagggagcccaggcctgccacctcatatgtcgggttcagtgcgccgcgaaggctctccgcgcctgtatctcggaggacccccagagcgctgcagtattgggcgtcagggccgagcctctcgcagcagtcacgccgcagtccgaggccacggacatccaatagagcccacttcaacttgcatggtgcatggggtccaggaccggtggcccccacacagggtttcccaaagcctctccatttgtctgcactctccactgccgaccttgtaggaaatttatttccatgcccagaaatattgtttggacacagtggctcggttatcttcgcgcctgcttttcttaaatacatttatttcacctctgccacgctgcagatatttaaagatctgtagataccgtaaccctcagcgatgcgggactcactctggttatagataatattctctgggtggttatttgaacataagttctatctccagcccagatgacacccaagcaggggctgtaaaggacacttgctctagaatgttttcaactgaaatatatgtccacacacggagaatttaagagtatttttatatttctctctagatctaaatattcagatgtgttaattacatgccctagaagctggaagcgatcagtggtgttcacactggacgtggagctgtttgtataattttcatctccctgcacttaaacatgactctcagtctaataaattcaaccttgtcatttttagaatcgacgggatttctctggctgtcgtttgcgctgcatttatccgaatacatccagctcgcaggcatcctgcaagaaacggctcccggctcgcgtgtacgccgacacctcggcccaacgcaggactcgaggtggtttctagtgcccgggtggctgcaagtctgccctccgagggaggctggacaagcggcgcccccaggtcgagcggcctctcgctgcctggcagtgcctggcagcccccacctctgccagtgcttcggaaacccgcctggccaggttcgcccgcggtgaaaaatgaaagcaaattccccaacagaggtagccggaactttcctcgacgaaggctccctcctgcgcctgtgtctggagaacccccagagcgctgcaagttagcaagagagattcgatggcggctctggaaggcgcacgaagggtggggcgggggagcaaagaggccactgggtgacccagcctggtccggggtgaaacgctagagaaggcgcctcgccttccttatttcaatgcaactcctcggccccagacggtaaaaatagtttccaagctgccgcggacgcccaggatgtgtttgcagagatcaaactggggaggaggcagctttgtaaaagttgcagaaagattagccgagaagcgtccgcccggcggggctcaagaaagcttggggacagctccatgttccttggggcggaatggcccaagaattggcctgggtaaccccctgcccagttctctgtcctcacttcgagccagtgcttaaataacttcccgccgcctgccctgcaaacttcccggcgcggcgccgttgaggccaggacacagcaaaggctagcaaaaccccgccgcggcgcgctcggccccggccctgagaggctgcgcgggtgggaggaccagtgtggtttctgctcccactcggctgcccagaccctcaagaccgatcccccaactcctgggagcgggtgcttccctctgggagccgagatcttgcggggccagcgagggcgcaaagtgcgcgcgctggcccgcgcgggggcggccgcgcgtctctccgcggggcctgtcgccaggccggccgcggcgcgtgagtgatgagggcagagaagggcgcccataaatcgcgggtgtcagggcgaaaaactctctttattgtctgcgtgatggatgggcccggggacgagacaccaaatacttcgtatcgcctttaaatgggaacacattttccgcggccataattcatgttttttaaatagaaagtttgaaatgttgcctatatttcaccagccctgacatatttatgaatcgctccctgcatgcaaatatcattacttaaagcgccggggagagcgcgggggaggggaggaggcgcggttcccggggcgtgggggtggtgaaccggggaaatatcggtggaggggggccatctccctaaagggcagagggtgaggtgcggtgaatagccgtatccggaaaccgagcgtgccccgggcttctttcccgccgccagaccccgcacagccgccctgggacgtttttcgaggcttgggacctaagacgggtccccggaccctgctgggaaaccagggggcgtttttcgtccctctctgaggccattatccaagctgaaaccaccttttataagaaaaaagtttaaagaaaaaaaatttaacaaacacaaataataataataacccaacatgtttcgcgttctttgccactgcatcgattttattgattttcattctgcaaacctgggaatgtgcgcgctcgggaggggaggctgtcaaaacccagtcatcgcggggactggatgggggacaattctccgacgccgcggcctggacccgcccccacccaaccggcgcaccacctcccaggcaccacggagcctgcgcaagctgcggggccgtaaaggaaccggcccccaatctccggggcttcagtcccgcccctctctccccccagtgccctgcagcgctcccagggtccgtgtctgccctccccaagcccccactgtcaatacaatccctgttaaaaaaaaaaaatctttttagagccaggcacgtgccgtcggcctctcccctgcccgcctggcccaggaggcgcccacacctgcgccctcacttgccgagacttttacctgtgcagtccccggcggcgcctgcagccgcgcaggtaaccgagaaatggaagggagggctggggagtgagaaggacagagagcaccgcccgacacaaccagacgcgcggacccggggcctcctctctggaccggtgctggggccctggtagaaccagctccgggagccgaaattgagccatgaagagtgggccgagggaggggcgaggggaacgggcctcaggagggaggggcgggcccggcggcacttccgttgggggtggggctgcgggctgccgcgccggtcccgcccctccgccgcgcgggcccgcccgctccaggccaggctccgcccccaccgactcggagctggatctcaggccacggcgcccccccagtcgcgcccccaaccccggaggcctcgggctgaaccgggtttcgcaggagggcacagggcg

SEQ ID NO: 232 - DNA, 4440 bp, Homo sapiens
  source 1..4440: mol_type = genomic DNA
cgtgaacgccctcgcctcgcacctgtggcgcctgccgcggggcccgaccggaggcagtggctgtggacaagggcggccccggcgtaggccaggatttccccgcagccggaccggcccagctccgctcctttcctgggcgaacagcgcccacctccggcccaggcggcggcgcctccgcccgcggcgcggacagcgagagagaaaccggctcctccaaacctggtggaggagcgctgcagagaagcgccgggctgaggctctgggggccgcggggcgcgaaacgccgcgcctaccaggttatgcccgggagaaggagacaggtcgggctccagccgctgggcgctcctcacctcgaagcgaggaaaacaccgggccactgagatcaggggaggtgcgcccgatacaggtacccggcacgcagacacgcccacccccaaacaccgagaaacaaagactgccccgagccacgcacgcctgcgcggccagcgctgggcgcacacccgcgcccacacagcccgaagggcgccacgcaacccgcaagaacaccagctccccgcgctcgcaggctctccgtgccggcccgcagcgcagagcagtcccggcgccgctgcctaggtgggccggggaaggcgcgccaccgcgcatggcaccgcggctgccgaggaacagagggtgccctgcgcagcccagctcgtcccaacccctccggtgcccactgctgcgacgcctcgcaggccgtgtcccagtgggccctgggcacagcggtcactcaccttcctgcacagcctggaatgggttgttggcctcgatgaccttgatggagtaggtgatcttctcgctctgcaggatgtcatcgttgaggctcaggtcggataccgccaactggaacaccctgtcgtccttggccgcgttctcctcgaagatggcacctggaggagagaagggatcaagaccggacctggacaagaaccctctccccgcttcccttggcccgagggtcaaggcactcgcagaccgatgattgccgggtggtgggaagtctgaactgggatttcagcttggcggggtagattaaggctctccgatctccacctgcacaggaatatgttcccctccccccaccagccctcagcgcacacgtgcacacagttgacatgagttacacatgcacatcccacaggctgccaccaggtcacacacgtgtctcacaaccaaggcaccacatgtcctagccatgctccacacaagggactaagccaagctggggtggccccaggcagcgcccaaatgccttgacatggttccataaatcccatcccaggagccaggaagttccccgggagggtggtgtcccccacccaaatcccaggacagaatgatgggtccttctccatgcacgtcccctgctgagtggagctgagcgaagcaggtctacagttggctttcctgcgcagaggggtcttccttaggcaggggcttcggatggcctgcagcttggatgactgtgacgctgcccggcctggcgccctgagctcagcctcctactctccatccttccccactcccatttcctgtacgtctgtcgaggggagtgggggaaggggacttgtgtcctaggcgcctccacgttcagatctagcggagctgggggtcccagaaggagctgctcttgaagggctttctagaaccgcggacagctcctccagggcgccctccggctggcgctttgtgcgcctagcacagcttccacggaccctgcgcttccagccgagatcagtgcgaccagcccgagcctagtcctgcaacctggacagctctggagtcagccacccacatcccgcctcaccaagcctcgagggtccctgaggtcccggagctaggcccagtgatccctcgagctgggggctgggtaggaggagggaggcccggattcctcactacacccggagccggcgccgcaatgctgaccgcgagacccgcacccctccagggcgggaagcaggtttggtcacgggcccctgaggaggaatcgtagctctccgagcccaggcctagtcctcaccaccccactccaccacccaccctcccgccttgtcccgtcctcctcgccttcaacacctcccaacccccagtttccctgtgctcgctggtctttcccaacttccggaaagacgactgcggagggactcccccaaagcgaccgctgtcagccccccaactcggcccaacttcccgagattcctcccgtgttgctcccaactcccacccactccccctcggcgcgcccactccccctcggcgcgccccctcctcctctgcgctttcatcttctctcccggttctctctctctatccatctcttcccgctcagcatccccctaccccccgctgcccacgcttcttccctcggctcccaccacccagaccgtctgggcccccaagacttggaccacgcaaaactctaggactgtccaggatggggatgcagtcgccacaaccagatacacacgcactcacacatacaccaagggctacacagacacacaccggagaagcaggcagacactgtgtccactctaacaggctgacagacagtcgtgcacgcaccaacacacgctcagatggccccacacaccccaacaccctctcacagtaacaccgacaccgtgccaggcgcacacagcgacacacgctgacaacacgcacccacacacattcacacacggttccggtccggcctctttgatcttgccctgccaacttggcgagccccccgctgtacctccccctgccccccgctgctctgagctgcgcagaagggccctcccgacccgccgacggccccgctaagggcgcgggtctcgacgcgcgtccatcccggtgttccccgaagcgtcggccctaagtgtcggtagaggccgcggggccccgcgcggagatcggagccccggggagcggcaagcagccagcgggagcgcggcgcggccggtgcacagctcctccgccgggcggcgcggcgcggcccttcgggggagcaccgcccgccgagcccctcggcccagggaaacgccaagtttggacgccgcgcaccccctgccccgttggggcccccgcccagcctcggcccggcctccagccgcgcttaccgatgtggatgatggagtcggcccgcaccgacacgcactggcatatccaggggagaagccacagcgtcagcgcttccatgtcccccgggcgcgcggctcatccacccgggcccggggcgaggccgagggcagcgcgaagcggggaggcgctggtcccggtgcagtcccgggccgctccccgggagagccgagcccgcccgtgcgtcttcccccgcgcgcccgcccctgcgccctgcgcccgccccagcccagcccagcccagcccagcgcggtcgggctcccgctccggctccggtggcggctgcggcggtgctggcagcttgagcccaggccggcgcggcgggggcggcccgcggctgtggcagcggcgcttcccgcggctagagcggcttcgggggaggctgaggcggtggcggcggcggccgggccggcgtggcggctctgggctggctgtgtgtctgagccccggcgaggagcgaactgcggaggacgccccctcccctccccctccccctcagctccgctccccctcccctccctgcccgcctccctcccctccgcccgcctctctaccacgtgactgcggagcctcagcctcgccgcgcggctctcgcccgctcgcggctcggagggggcgccgggtgctccacgctcccggctcgggggacgctcggagaccggcacagcggcggggaccggcctgggctgcggcgcgccgagctctgacggttcgggcctccttctccggttcgccaggggcgggagaatggaggagggccggccgctaactgccctcgaggggctcccgaagcgccctggcgacctcagcagccgcgcgtctcgggtcatgtccccggccgaaacagaccgttcctcgtggagtgggacctggggagcaagaagagagaccggggcggggcgccgggccaggccccaggacagctggggagccagagggggccggccgccgcaagcatctcagagtagagcccgggccgtgggctcgaaccctggctggccgcgcggacctcggcgggcgcccatcccccgctccatgggcagcgcgctcagccgggagtcgggcaagaggctcggactctgaagcccaattcatggccggaaaccagcgcagccgtgaccgcggcgcctggacgtgaggagccttccaagcggccggcgggctcagactccgaccctgcacggagctttcatgtgcgagcccgggcgaagagcccgctcctcgcggccacagcggaacagggctccctccgcctccggtgcctgcctcgggccg

SEQ ID NO: 233 - DNA, 408 bp, Homo sapiens
  source 1..408: mol_type = genomic DNA
cgagggtttgatgatgcggcccgagcctggctgtggtcgcctgtcggggctggagcgggaccctcagccgggccgggcctgggggctaacgttttcacagtgcgccctgagtttccttgggttactgctgggaccgcgcaggaggaagcaaagagtttttcgagctagaccaacaggaaacacattgacggaaatgttgccatagcccatggggtggctttaactggccgcccccgcgggctgggtgtgaaatcagaggaggccgcggctcccccggccaggattggaggctcctcgcgcaacctaatgcgggtgtccgggcccgagcgcttcccgcgcagccaggccttgtcggtgcagcagccccgctcctccccaacacgcacacacccggtgttcgcaagtgcg

SEQ ID NO: 234 - DNA, 2237 bp, Homo sapiens
  source 1..2237: mol_type = genomic DNA
cgcgcccgactgcgggagccaggatcaggtccccagggcgcccttgcagacagcaccctgagcgaaggagctcagaaggcagtccggcttcacccagacccccaatcccacagaagcgccgtcccccagcggcacagcaccccctccccggcgcggtggtagggcccgacgtgacgtcacccattgtttacaaatcaacccgagccggcaggattccggctcccgcggctgcaggcgcgcggctagagtgcctggcgggctccggcttccgcgtccgccccggccccggtccagacttagtcttcagctccgcgcccgctccgccgcggcccaccgcgcccgccggcagccgagcccccagcgacgcccgcacagctccgggtgcccagacagggggccatgccgtgccggagggaggaggaagaggaagccggcgaggaggcggagggggaggaagaggaggaggacagcttcctcctactgcagcagtcagtggcgctgggcagctcgggcgaggtggaccggctggtggcccagatcggcgagacgctgcagctggacgcggcgcagcacagcccggcctcgccgtgcgggcccccgggggcgccgctgcgggccccggggcccctggctgcggcggtgccggcggacaaggccaggtccccggcggtgccgctgctgctgccgcccgcgttggcggagactgtgggcccggcgccccctggggtcctgcgctgcgccctgggggaccgcggccgcgtgcggggccgcgctgcgccctactgcgtggccgagctcgccacaggccccagcgcgctgtccccactgccccctcaggccgaccttgatgggcctccgggagctggcaagcagggcatcccgcagccgctgtcgggtccgtgccggcgaggatggctccggggcgccgccgcctcccgccgcctgcagcagcgacgcgggtcccaaccagaaacccgcacaggcgacgacgacccgcaccggcttctgcagcagctagtgctctctggaaacctcatcaaggaggccgtgcgaaggcttcattcgcgacggctgcagttacgtgcaaagcttccccaacgcccgctcctgggacctctgtcggccccggtgcatgaacccccttcgcctcgcagccctcgcgcggcctgcagtgaccctggcgcctccgggagggcgcagctcagaactggcgacggcgttcttgtgcctggcagctaacacgcccggggtggccacagcgccagcctcagactggagggcaaggggttcccttgagggctgcagttctactcaggctggtggagaactctggcttttggaagcgagagtaaaaagctaatgacgaggaaccgaaaaatcgcgagtgtttcgcgggtaactggggttgagggccaaaatatttggaatgaaggactttggccctatttaaggcagattttacagagcgcacctcaaacgtacaagtcagtaggactccttatttggcgtgacccgacctggccgcggagcctgcatttcctcgcagcctctcagtgccctccagccccgcgaccatgtggccacaatccacgcttctccggatcgcggtgcgccggaaccacggaggatgatgccagttacttgctttaccttttcagggctggctcctgatccactttgggggaggagaacatgagtagataatttcagggtgcagcccaatctgccagacttaaaaaaaccatcttgtgtctttggaggtgctgcttaataccaaacatgcggtgccatgaagggaccctttgggggttgaataggagttaacccctgcgctctctttgcaactgtctctcttctcagagtggtgggggaaggctgtacgacacgggtggggaaaggaggtgggggcggggagtattgaatggtggtggaagggtagagaggcgcggagtgaaccccacgccctgtctaaagtgtattttcagagccggcccgcctctcctcggttcaaggtcactgtttcctgggcacgcactgggttgcgggacagagtagccaggttctgccggtgctcggagaagagcgcagtgttttgcaagtgctggagtctcctgaggacacgcgcgtcgccgccaccgcgggtgtgggaaagcgcggacgtgctgggcggctgtgcttcggtaggcgaccaccgcccctggccgcgctccgggctttcacggaaactcccgagaccg

SEQ ID NO: 235 - DNA, 758 bp, Homo sapiens
  source 1..758: mol_type = genomic DNA
cgtgcacgggcttggagacggggcgccagtaccaggcagggcacaatgaggcttggggcccttacagtcgctgcaacctggaggtaccgaaccggggactccgacctgacatgcaggcgaggctgcgaggtccagagcctggacctggttcctagggagcgccactccttctgacccctgtggcgtgcgtcctggcgcgccgcacccgcctggtgcctcggccctctctgctgttccctggctgattctgagcaaggccagcagcccacggccccacccgacccccacatctccgcagggtcaggcgagccgagcaggaactgcgccggggcagtggtgatggctgggcgtccctgcagcttcaggggccaagggcaatcggggggctaggcccgcagacctggccgtccgctgacgcagcgaaggtctagcggaaccttggggatcgctctcccagtgccgaccgaaggcccggacctccaggctggacgcggcgaaggccgcataagacgttacttaaacatgttacttaaacaagactgcagtaaacgtttctttccaagtgagaaaggtctttttcgttctcagacggtttgaaggtgtttgtgccaacgtgacccccggggagatttggaggaagctttctacgtcctaggaggctgagatcccacggagccggtttacggttgagagcagacagtttcgagtagatagcgctggaagagacacgaagaatctctcttgcttcttggaacgcg

SEQ ID NO: 236 - DNA, 5881 bp, Homo sapiens
  source 1..5881: mol_type = genomic DNA
cgctttaactagacgggcggataaatggccccgtttctcctcagccccgacgcgcctgcttagctgcgcgccccgcgggcgccgcagctcgggtgggctcagccctggggccgccgcccccagcccaaagggacccagcggggccaggcccagggccgcgaagcagagagcggccggaggtgccagggccgcttcattgggattcatgagcgtgttgggcgggcggcctggggcttcgggcgcggggcgggggccgagtgggggggtgcgcacggcgggcgcagatgcccgggtgaccgacccgccgtggtccctgcgccgcctttgtgccgaggaggcgcgcgcggggacctaatccatcaaattgtctacaaattgtgaagaaaattcaaaaaccatatggtcgccagcgccggcgcgctctgggctgcggagggccgcggccgtgcccgcaccgggagccgcggggggctgggcccggctattgtcgcctgtcagcggccgcccaggcccattcgtcccggccttcatggtccgcgctccgaattacagacccatccatcctaagagagggcaatttttgtcgcccgggaggaaccagccgcacgcctgccccgctgccctgccgtctccttccttctctgctccttctctctcttctcctctcttcccattctttttttctccctctgtctctgtctccctttctccgtttctctgtttctctctctttttcctctttttctccgtcctgtctctgtggctctccgctttctctctctgcttttggagtgtctttctgtgctcctatttttttttcctgtttctctctgtctcttcgtttcctcctggacacgcacgttcctggctcgggcccgggggccccatcgccgcccgagaccagatgcgcatttctcacatcaattttgcggagaaagagagcccagcggcccggaggcgggaccccactccggtgacagcggctactcgccagcccagcgacaacaaaagcccggagagaaacccgctctgtccaggaaagtgaaagcggcctcagcccccgccccagccggccttggggcgccgcagacccgagagagtccttgcaagatccatacctctgcccccagcggtgtgccggggtgtggaggcagaattggttaatgttgccagaaattcacaccagctcccagacgcttgggcttacagttccaggtgcccacattgaaattcagcctggccttgatgcctttagcctttggcccccgactccccaaggatgaggccctcggggctagaatcgaaacttgggaacccaaggctccaccccgcgtctgactgagtgatacctgcagcgctcccggggctcagccctgttttcggggagtgcagggagaatgcccttgggtctggggtgggatgcaggaaatgcagggcaagaccccctcccaccctttgttcaacacaaattaatgttgtactgtggtgtgcccccacatacacaggcattttctcccagcatttcacggctcgttttttaccccgacctggcgaagcccgcttctctcccgatttagcagctgcctcaggtttatgggcaagaggcgcctcctcctgctgcttccccctcgcccttcactcttaagcctgtcctgaaaatcccccccaaatctccaaggctagggcgaccggcacgaagagcggcaagaagcccagcctgccgctgcttctcggaaacgggcccaccactcagacacggtcgccgccggttctagtaaaatggcaaagactttatttattggagaaagaggccttgggtacagtctagggaggagaggggaagggaggagcagggaaggggcactcgcagcccactcacatccagccaccctcctctggccaagtcgggtaggagtggggtgttcgaagtggcaagagagacccagagctgtggaggaggtgaggtgagggcttgggtcggcctcctctctgtctctgtcacatgcaaaaaaattctaaaaagagtacaaaataattatagggctttgggttctgttttcgatagttggttggttggatttccccctgcattgtccctaagagccggactccaacagtcagtccgaggaaatgcgggagggagggaggaatcttaaaaacagcattagtgggcattaggaggcagaaatggcatcgaaaacagacttttctctcagagggtggggaggaaagatcgattttgaaacttgcaagtcaggacctgactgcccacctcttcccctcccacatgaggaaaaaaggcccgaggaaatagaagtcaactgcaataaggccgagggaggcgaggaaggagcgggccggtgcattcgccacatgcagagaaaaatcaaaacagccggagctgccactcccctagaccgcagggttagtgttccccgccctggtttctgtctcgccctcagcaaacccggcagcccgagagctttgaaaacatcgaaatctccgagatttaaaaaataataataaaagaaaggaaaaatcaggctgcggtgggaagaacccgctatataggtttaaatatttatatagaagccatacagaattgcacggcgagggcttccggggcggcggaggccgctggtggcggccggcccggccggccagcctgggtttattgcttcgagagggaagaggcggctgccaggagccgagtccggggccggggacaggggaggaggcgggagttggcagaggaggtcccagctcccctcggcggtccggtccgggaggcgttgggctttcgagcgctaggagcccagggcggcggaagacccggggcgccgctcaatcgtccacgtcgatctcttcgtcttcctcgtcctccgagcagtcctggctgctggccggctggtccgtgagcggagaggcaggcgagagctgcagggcgccagcgcccggggccttcggggcgcctggggggaggaccggagagccgggcctcgacttggccctgccgcagccgccgccaccgccggctgtggcctccgagttctgctcgagttcggccagcgccacgatgtccatctgcccgctggggcccagtttcttggcggactctacgtcggccttcatctcctccaggtcccgcttgagcttagcgcgccgattctggaaccaggtgatgacttgcgcgttggtgaggcccagctgctgcgcgatttggtcgcgatcggcgggggacaggtacttctggtatagaaagcgcttttccaattcatagatctggtggttggtgaaggccgtgcgcgactttcgccgcttcttaggggtctgccgctgcccaaagatggtcataccgtcgcggcctgggaggaaaggacagtgtaggctcgcgttgggagagaggaagcccaccctggctctcgcccccgtaactccgtaacccacccaggcctgtccggcctgcagtctaccaatgttcccttctcagatttaattcagcctccaggaccagggcaagtcttggctatggcaaaccatctcctgtctttggtctcccagaagtccctaggaaaaattgttttggtctctgccccctacctaagagttggtttgaaagacccaaagtctctaagggtatgagaaagccagacagagacaccctaattgcggtgtgaatgttggttctccatcccttcccaatcacttcaactctaccctacccataaggtccccggcaaaagactctcgattccccacgccgagtagccgggctaagagcgcccaggacactgggaacgcaagggagcccggcaaagcaagcagactgcggaaagagcttggccacccttctcctgccaccccccagctctggcctacccagaggtccctgggaaataaggattgggtcccgtcccacatatcttcactcccataagcagctgctcaggctggaaggccctggcggccggagagggcagaggcgagcagaggcataggggacgaagcgcgcagggcacagggcgcgaggcgctgggtgcaggggaacccagcagctgcgcctaccttcggctgcctgcagaacgctgacctccagccccttaaacgtcttgctggcgagctcctccagcgcgcacagcggcgaggtctgcgagagcagcgcgcggcccgccaggggcaagccgccctgcgcgtgcttgtccgcggcggccagcaggtgcgccgccccgcacagcgagtaacttctccgcacagacggcttgttgaggatgtcctcgatgctgaacggcgtcagtggcttgttggagttggcaggcggaggcaggtggtccagcgggctgcgccgccgctcctcccccggcgccgccttgccgtcctccttggaagtcatctcggccttgttcggggtcccggccggggcggctcgggccgcggggacccagcccgcgggcagctcgggcgccggacggcgcgggcaggcagcggcgggtggaaaggaggccgcactgggcagggcgcgggccgggcgcggggccgattagcgcggcggctgaggaggggagaacagcgcggggccccgggaagagggcgctgacgcgggcgccgctgctgctggggcttccagcaatccggggccggagccggggcgcgctgcgcggggctccagtttcgccaaccccggtgctatttaaaggtgtcaggtggctcggcagtggctcctggccccgggtcctggcggccgcagttggaagagccgaggcgagggcgccgatcccggactgcgagggaggcggagagacggggcaattgactattgctaacaagttagccttgatcgcggtgtaatcaattatctgcagcggcacttctttctctttctctctcctttctccctttttcctctcttctttgcctctgttctctcctcctgttctcgctctccctccctccctctctcttctctcttctctgctccccccttctctctccttctctccctctccctcgccctctctttcactctctgtctgtccaatgcaggcggctgtaagttgggaagctcattaattagcgggccgggggtaggaggagccggctggaattagcctgcctaatgcgcctgtcagtccgggcgctgcgctgcgttcggcccgagctgtttgtaaattacattagctgctcctttattgcacccgaacctcgggcgactgaaaagccaccgcccccaccccaaactgcgagccgcgctcctggcgcacccgcctcccgccggcctagctgcaatgaccgcaccggcccgaaggtctcggtctctccgacccgggatgtggagcccgaaagagtggtgggaaccccagcccgggagggacgcggccgccgctcgggccaggtaggaccaggagcccgggtgggccccgctgccacccagagtggactttgcggagctgagatcaagagcccagcgcctctacctctgaccctcttgcccctcaccccactgcctgggcgacgcgcctggggagagggaatctgaagccctcctgcgctggcccgcaggaggtcagagccggctgcgcagccttgggtgggcgacgcgggaagggggtggaggaccccaggggttgggggagggggctagtcggagagctcagccttctccacgcggagaggcggaggagggaattgttggcggctctggacgcctctccccggcctgattccgaagcggccgccctacctcctccggcaatcttgctcactttcctccggcagccgctccccaggctggctggggccaccaacggcctgatgatgggcgtctgaagagtagccaggccaggtgccggggctaacggggccggctccgtgcctccctgctgcg

SEQ ID NO: 237 - DNA, 3245 bp, Homo sapiens
  source 1..3245: mol_type = genomic DNA
cggtcacgaaggatctcagctgcaccaagcgcgcaggctacactcctgcagtagcggattggggaccagataagcccacgcggctcccgcctgagtaccctcttaactggcggccgacactttgaaacaattgagtccgttcagaaacggactggggagagtgagggcacgacctcaaaatcctaggcagcaaaatgaggtctggatcctccctggaagaacagacatctcgactactctctcgccccacctctggccgggaggtcggggcaaggagaatccctcaaccgagaagatctggcaagtccccgatcccaccggggccttccgcctcttttccctcatcccctaggaggctttatcaggttctaaccgcttgtagtgggggccgtgggtgctcggggcgccaagaggagggggctgccgagagcggcctgcctagttctcagcattgcctctttgaccactcgcaggctcctggaaatgcaaacacagcccgtgcgtgatccgcacgcgaccaaaagccgagcgcacacctcccgcccccgccccacttgccatcgctcacactaacgtgcacacatgctcgtgcaaacgcgcacgcacggaaccgcgcgccccctcacacccagccgcgctcccggcccggctcagccaggtgcccccacgcggcagcgcccggtctgcccgccggccccctgcgaacccctcgcctcacgcccgcctgcctgcgcgcacgcacgcgcacgcacgcattccccacgcccccgcgcgggggcgcaggccttgggtggcggggtttggccgggagccggccgggccagggagcggcgagcagagtccagggtgcatcccgcgcttggcttccgtgcgcagcctccccgcgcggaggcacctggctccctctcggctctagaacgcgaacagttggcgttgagcacacaactcctcctccttctcccccgagacccctgtaccctccccgcgcgccatctcccgcccctcataacaggcccgaagagtaaacccccaaacgcgtccagaagcggctcccaactcttcgccgggagcacgttggaggctggatttcggagagactcgggattggggtctattgccggcccctcctggatccggcttgattttcatattttaagaagattttcttattacctttgattactcctttttaccaaatttctggaggagctcagtggggaggattggggggaatctgattttaagaaagaaagcaccgaaggggcaattattaattttcctcgggtttggaatcaccctctggacaagagaacgggcgagcgggagctaggagggaagagtggagaggaccggcgaggcgcgccagccggagccacctccttcccggccgccccctccccactccccctacacacacacgctcgctcgctcgccggcgcgcgcacaccccccgcgccggacccgcacctcggcgggcgccacacactcggcagcccgagccgcggtagccgcagcgggatggaggcggcgcgcacggagcgccccgcaggcaggccgggggcgccgcttgtccggacggggctcctactcttgtcgacgtgggtcctggccggcgccgagatcacttgggacgcgacaggcggtcccggacgcccggcggccccggcttcgcggccaccggcgttgtctccactctcgccgcgggcagtggccagccagtggccggaggagctggcgtcggcgcggagagccgccgtgctggggcgccgggccggaccagagctgctgccccagcagggcggcggcagaggcggtgagatgcaggtggaagccggagggacatcaccggcaggcgagcggcggggccggggcatcccagctcctgccaagcttggcggcgcgaggaggagtcgccgggcgcagcccccaatcacccaggaacgcggggacgcctgggccactgctccggccgatggttccagaggaagccgtccccttgctaagggttcccgggaggaggtgaaggcgccgcgggctggggggtcggcggctgaagacctccggctgcccagcacctccttcgcgctgaccggggactcggcccacaaccaagccatggtgcactggtcgggacacaacagcagcgtgagtacccacccggcggcgggtccgcctgtttcctgacaccgaaggggaatgggggggtgggtgggagcgagggacagatagttgctcgggggtcgaggcgggggacgcctcgacttttcgagataacaggtttgtccgattccccccacgcccccaacaggttaacggagtttgttgttagacgctgtgtgtgtttgtttactggagaccctaggcttcggccgccgggaggggagtggaggaaggggcttcttaaagatgagtgggggagggatctgtgctcactttctccaatacttggcttggagggtcagttttcctgtttgcggggtgcttgaattcttggatgagaaaaagggctgacttggggcgggagccgctgaacagaccgattcctgcggctgccgggctccccctacccccaccccacccccaccccccctcccgtcacgcgaaagggaacccggaaggccattcggactcccagcctcctgctcgcctcggggtcgctgctatccgctccaggcgcgcagtcctccagcccaagagggaggctcggccaagtcggcccccagccttggccttcaggtaaccccggttcctccttcaaagtccagggagcggccctggaaagccgtagcagaggccgtaaaaaaaagtttaaagcgtgaagcgaaattccacacccatccagcgctcatgccacaccgctcgccctcacgcacgcacaggcacacggaagtcgctcagcagaaaactctcgacttcaccattgcgtcctgcgccacaaacggctccctgagcggagttgggtgcctgagcttcctgcacacctctccacccgcttttccccgccaccgggttctcctggacaaggatgtaccgagaggtggcgtcgttgagcccggtctggcctactccggcattccgaactgggcgcccgactgagcatcgcgcctgcctggcagctgcagcggcccgcagcgcgtgcccggaggggctccccctacctcgccggcacccagcgctgtgcagccaagcaaagaaaaggcgggggacaggttcccaccatctccccgacttccctccctcccgcg

SEQ ID NO: 238 - DNA, 710 bp, Homo sapiens
  source 1..710: mol_type = genomic DNA
cgggcgctcattctgagccgcgacgccccctcaacaggctcccggcaccgcagagccagaggccacgcggtgagaaccccagtgcgtgcgctcttcttgtgtgagaggccggaggtagagttgggagaaagaggagccggcgaaaaggttagtgggatctgctttcccttctgcggccttctgggccagctagggcctgggagatggggaccagggtcggaggcttgggggcgggagggcggtgctgccggctgcttcctgcaagcggtttgaagaggcatcgcgttgtccggccgcctgcgcaagggccacgcgctgctgctttcacccccttgcgcccgcggggcacctggactccccttaagagtccccgtggggcctgagcttctgctgggcgctacgctgccctccacgattccattttgccccatttactcgggtcgtccacttcccgcggctcagtgacccagccagggggcgtcttccgctagcggtccaaatgtagactccgcaaagaggctaagaagacctgtctacccgggtgcgcgcgatggggtcagggtgtaagaaccaggagcctcatagaaacctctgcttggggtccaaagcccagccgggcactcccagaggacaacatccgcccccgcatgtacacgtggccccgcaggacccagccgcagggctcccacgctcggctccccgccctctcg

SEQ ID NO: 239 - DNA, 474 bp, Homo sapiens
  source 1..474: mol_type = genomic DNA
cgaccacttccgtatgaggcacgattcgttggcattcttcacctgcagcaaccgctgtgggactaggctggttccccagccccggcggggtgtgagtctgcgacggctgctcactacattcgggggacaggcactgcatcgggggtgagcaggccagggcagtttccgagggcgcctggagcccgttgagatgtgtggctctcctccgctcgtcatctgccgggcagagtgaccactggctggcggctgcagagtctgacaaaccaggctcgggggccctggcactgagggcaggtgcgtaaacggtggcaacctccgttttaaacaccctcctgagggcaggtggagcgggctcctcggagtttggcctcccccggtcctgcttggccctcccgagcagctggtaatcgggctcttcggatggaggccgagtggagttttggagcgcagcttctccatagctgagcggctccg

SEQ ID NO: 240 - DNA, 515 bp, Homo sapiens
  source 1..515: mol_type = genomic DNA
cgccgccgccgcggccaagggcacctgcagaatgtgagagagcctgggaggtcttgcagcttggtgtcaccccaccaccctaatccccgagggtggggggaggtcgacggccctcctcccaaccccgacgggtcctgaaaaatgggaatgggtcagcatcgggggtacctaagccgccaagtccttgactcgcccctgctctgccgcgcagggtgctggagaagaagcaagacaccggggagacaatcgagctgacggaggatgggaagcccctagaggtgcccgagaggaaggcgccgctgtgcgactgcacgtgcttcggcctgccccgccgctacattatcgccatcatgagcggcctgggcttctgcatctccttcggtatccgctgcaacctgggcgtggccattgtggacatggtcaacaacagcaccatccaccgcgggggcaaggtcatcaaggaggtgggcaacgtctggccgccctggctcctgcccttcggccatgcggcctcg

SEQ ID NO: 241 - DNA, 1356 bp, Homo sapiens
  source 1..1356: mol_type = genomic DNA
cgggaacgtgaagcgaagctcaccaccggtgcccgctacccccacgccggcaactctgtgcttcgtttggcccacgtgtcctccccacgcaactaggggcgacggtcctgtgcctgaggtgacaatcaccccccaaaaacctcacttgcgggtgggggggtggtgattgccagttgacccagaattttctagcaactaggcgcgttaaagtgaaaagcccctggactagctccacctccggccccaaccatcacaggcggaccctagctgatttgactctcacttcccgccctcagactgccggcgacgaaaatccgcccgaatcgggcgccacctctggtggaagctgctgtcctcggcttctgcccaactccaagaagcggaaggggggtgtatcctgcgccccaagctcatgggagcccacgcaccggggagagcccggagtggaggctctgggctcagccctcccaggcctaggccaccgtgccctgagtccctccccaagacttctcggactaagtgggccagactcttgtcaggggagggatcaccaggacctcagattgctgggggcggaggggtggacgctcgtgagtgcgaagttcgggtgtcacttgggtgtgtccagtgtctacaagtgggaatcctacggtttcaaaagtcaggcagggatggggttgcctacactcggtggggtcaatcgctagtctcaccagaggcgggagcgggcctgggggccctctgagctcgcggaggcggcgggttcggggcccgtgcgggcggggtcgcccaggggagcccgggccccgcgcgtcaggaggcctccgccggccgagcggccgcgctgggaaacaagaagcgagcgctttgctccctatcttcccagtgtccgtcctatattgttttctgctcttaaaactgacatgtctaattggcaattggtgccgaatcgtgtccagaggtctcttgtggaacatttctacagctgtctccttcaactagacgcttattcatgtcgccttaatgagaaacaaaacgttctctaatgagcaattacagagcgacaggattgttcccataacaaattcttgcgagtgacagaagcatcctttgtaccagatatttcgcatactgttaccgattttcccttccctcgcccgactccatggaggcgccgggcatccggagccggtccgagagctcacggatgcgccggcagcctcctcgcagtcccgcgcgccgcccctggccccgagcccctcggctccgttgtgctcctgggagtgagacgccacgtcccattatcccgcatattatctccttgtcacgaggacaacaaataccgattaatcttcg

SEQ ID NO: 242 - DNA, 512 bp, Homo sapiens
  source 1..512: mol_type = genomic DNA
cggacgcccgggtgggaggctccttccaggacccaggcgcccaggggcaacaccgccgggctgcggtgcgcgggtagcagcgaggacaggaagtgcacgttgcaggcgggcggctgcggggcctgcaggagcgcgacgtcctccagcgcgctgagctctgggatcccggggtactccccgaggagctccccgccgtcgctgtcgccccccgcgagcttcgccttgacctggcccttgcagtcggcgcccagcgcggggttgaacgcgtgcaggacgggttccttggggtccgccgggcccaggtacgcttctttgaagacgtggaactcgtcctcctcgtacttcacgggggccttcaggccgatgttctccgaagcgaacggaccctcggggcttaagagctccgtcaccatcgcatcctcggggctgggcagcccgcccggcagagggccctcctccatggcgcagggcggcggggcgggatggcgggggccgcgcacctgcaacacgcg

SEQ ID NO: 243 - DNA, 3661 bp, Homo sapiens
  source 1..3661: mol_type = genomic DNA
cggacgggacgggggacgggggcggggactggcgggaggggggcgggacaggctcgctctcctcctacttagcgagctcccggggaaaagcaacggtgtcctcctaagcctgaggccaccgcgaccgagccgagccaggctaagggaccagtgtctccctgccccccccccacctctaggtaagagcgccccgtcccactacacttgcgggtcccattcgctgcggtgctaggactggataaggggaagtccccggggcctggcgagagccctgagatcagctctaggctagggagctcggcagaaacccgtgggggagagagggcaccccaggtgagtgcgtgggggcgaaaggagcgttatggggagttttcctgtgagcctcttgcttttgccttcctcggccgctgccaacgccaccgccagagggggccgtgacgctctcccccccttggagaaactctgatcctggccctatccagactctcccgccggcacccaggcggccgggcggacactcgcggggtatcgggcggcgagcgcgcgcgcgggttttcgtcctccagcgccccacacctccccctctccgcccacagcaggaccttggggcgggggtatctcctgtgacgtctccccgcctgtccccaggccggccgccttgatggaccaggaagggattcggagtccggctcagagtcttgagcgtctaggagggctgcctgggggtgtccaggagatgggaagcccgcctggggcctcccaggcactccccaaggccccacgtcctcgggggccggggaggccgcctggggcgccttgccacgcccgcgcggtccctattggaatccctagcggagttccccgagcggaggctgacccaagtcatccgggctcccccgggatagggaagtgcgggcgggcggctgggtaggggcgccggcgcagggtggccgagtcgcccgctagcgcttccgccgaggcaggctcgagtggggacttggccgggcgaccgcacagccctgccggggacccacggctctggggcggggctgcattctgggccgttagctcagcggtcctggagcctcccgaggctgactcatcgggcggcgggctcaccccccagccgtcaaacgcaaacgcaggcgccccaccacgccgggctcccacgcggcacacacgcacgcctccgcgacacttcgcacacctacgtccctgcgtcacgccacccagacacacccggacccgcccacacgccgaggccttcccacgcactccatgccttacgcctcacaggaaagcgccccgcgctcacgcagcccctcacacccgcagccccgcttacacgcgccccagctcacacatgcaccccagtcacacagcctacctcacacacgcaccccagtcacacacacaacccagctcacacacgtacccgtcacacatgaggccccagctcacacacgaagcccagctcacacacgcagtcccagctctcacacacacaccccagtcaaacacgaggccccagctcacacacgaagcccagctcacacacactgccccagttacacagcccagctcatgcaagcagcccgtcacacacacagcccagctcacacacacagcccagctcacacaagcagcccgtcacacacacagcccagctcacacacacagccccagttatacacacacagcccagctcacacacgcagcccgtcacacacacagctcagctcaatacacagcccagctcacacacacagccccagttacacacacagcccagcttacacacacagcccagctcacacacacagcccatcacacacacagcctatcacacacacagctcagctcaatacacagcccagctgacacacgcagcccagctcacacatacactccagctcacacacgcagcccgtcacacagccccagccacacacacagcccagctcacacacgcagcccagctcacacatgcagccccctcacacatgcagccccagtcacacacacagcccagctcacaccgcagccccagctcacacacgcggcccctcacacacattcagccgcccttcccgcagctgccaggctcggaacgggcggtgtctgtgggggccgccccgtttcccggccgcgcaaacaagcggctccttctctctgccccgccaaagaaaggccgcttgtcaggggcgggcgggcaggcggcagcggcaggcgcccaacactgcggccttgtcccacgcgggcagcctgggcaggagcaggccgaggggtgcaaggggccgctaccttgcggaaacctccctccctcggccctgcggaccgcggagaggcacctttccaccaccggacacccaagcaacagccctcgctccatccgcgctctcccggctccccctctcctcgggccgcccatctctcctctcttccgcctttggggtcccggtgggccccagcctccctctccttcgccccatctccccgcccgtcagcctcccctccgctgccctcgcgcatccccagtgttcccgtctgtcgtcgcctccacttgaggacccgcctgtcctttcttcccccgacccctccacctctgtgttcttcagtctccccgcatcctctgccctcggtcttcccctccccagcttgcattttttcccatctcttagtctgcctttcattttccccttcacatcccggtggctccccttccccgccccgtccctctgcacacccccattcatctctgtcaggtttttctcatccttaagttctctcgtccccctccccactcgggcgtcccccgctgggctccccgcctcagtttccttcccttcctgactccttcctctgacggcggccctttctcgagggcggatgtgcgcctggagggcgaaggcggcggccgagaggaccccagctcggcctggcgggcctctggccacagccatgcaccgcttgggccgaggccgaggccgacccccagggacacaggtgaggccgggcgacccgcggggctccggggagcgcccgagcagtggccccgcgaagggccgggtctggtgctctggggctggctttggacctctcgtcctgggactccgtgggagcccgggagctgtgtcagtgcccgttggtgctggagccagtcggcccttccttggcctccttcaggcgggggccaagacccccgagagttcactctgggtgctccactcggcggagagggcttcagactggtgtcccgcgtggctggggtgtcggcgcattcccgcgggggaggaggccgagggcccgggccggggtcacctgccggcgccccggccttgcgcacagagggccttgcctgtcgctcgtcaaatccagacacctgggcctccgccactcggctgccggggcggcactggggcgtctgtacccagcgtgcgggagggcacccagggccggacgcgcaccgcaggggtctttttttggccgcggggccgggcgggcgggtaatgacagccgggttgctggggagccgcgggccagatggaagcggggccgcaggtgagggacgtccctcgcgtagcgccactcagccgccggggccagagcgggagtcaaggtgagggcgcccccaggtggccgctctcggccgcggcgatcg

SEQ ID NO: 244 - DNA, 1219 bp, Homo sapiens
  source 1..1219: mol_type = genomic DNA
cgtgcgggtgactcctcttcctgccgacaaggtgggcgatgagaacgaggaggcccagtcccaccagggcgccgccgacggcgatggggatcggcgtgttgttctcgcccagcagacgctcctccgcagatccgaactggttgccttccaccttgaaagcctggccccacactgggaatatgttgattgaaaacgccttcgtgccgcggacacgctccgctccggctgtagaattgccgatggctgcccgcaatgctcgcagggagtcgttggcagctttaaaagtggggtctctggggtcaggaagagtcgtattcagctggattcctcgtaggaaaatccggctagaacctgcattcatccccaaccggaagcccaggaccgtggtgccctcgccgcgccgctcccgggtctccaggtgggcgccgcgctcccgctggctgaggtcatgcccaggtcaccgtcgcgtggtccctcctctctgggtgaggttcagctgcagtcccgcgctggccggcaggcaggccccgtgggtgccgctcacgctgtacctggccacggaggggagaaggggctctcggcgccgtggcggggaaggcgcgtcttgctcgtggcgcgtctctcccttgctgaagctgctgttggaaatctaggcctggatcggggcaccgcggagcctgatggtctcgctgttcgtgcggacctgggggccttgatatctggtagagattccacagtcctgatttccttggagcttgcactgaggaaaatgtgtgtgtctgacaagttgtaaacaaaactcatgagctggacgctgtcacgtgtcgcacttcttgtgaaatggaaggtcagtgtctgtcttcctccaaagcacaatcgcgtgcctggggtcggaagtgttctctttactgcaagagccactgtcaagcacttccgcattggatggcaggtcaaaggtcacattcttagagccactcttagagtcgtagttcatcgagaaggcagcgaagctggccattacgcaggctgtcccgttgccctttttcaccgcatccgctgcggctgcaccacgcacgaggcccagcagcagcagcagcaccaggggcagccgggggctccaccagatgcagcccgggcggcaggcgcgaggggagccggtgtgtggggaaccggggcgcggggaggcggttgcggggcgctggcctgaaggacggtggcg

SEQ ID NO: 245 - DNA, 210 bp, Homo sapiens
  source 1..210: mol_type = genomic DNA
cgacgggacacggtacatcacaaagagacccgtgcgagaccgaatcctgaaggaacgtgccttaaagatcaaggaagagcggagtggcatgaccacagacgatgacaccatgagcgagatgaaaatggggcgctactggagcaaagaggagagaaagcagcacctggttagggccaaagagcagcgccgtcgccgtgagttcatgatgcg

SEQ ID NO: 246 - DNA, 901 bp, Homo sapiens
  source 1..901: mol_type = genomic DNA
cgtgttcccgggcggaggtgcgcgcagccaccccaggctgctgccaggtgcccgctggggctgccagggcgaggaggcctctgggctgtggagcgaaagtcagatccaccgcctactgcggggtaggggccgcagtggggaccgccagccctgtggtccctctcgcgctgactggcgtaaagttgtggccgaattcgcatctcttctggtgcttctcgcccgccagcgcagggcccaggtgtttgaggcgaaggggctctagctccccgcaagcctggagccaggcgtcgcgcttcctccgggcttaatccagacctttcaacacacacctcattcgggggaggagaaaagcacaggaccgcggagagcccagctttgaggccaggcctgaagggataacccacacagggaacgttttcctatcagagaataatggagcacaaaataattcagaaagcgaatgggcaggaccacagcctgagagtcccgcgccgcggggccgctgcagagccggtctcccgagcaccgcggcaggaccatttcgttggaatgtagggcgaggccgaagcccgccccggacccaggccgcgaggtgcgcgccggccgccgaggggccgcctgtaaattacagcccgccgggaggactcggaaatacacaaaaggagccgaaagatttaaacagtcggaggcagaggcgtcccgaggcggccaaagcggaaatcaatcacgtaattaaaacagggaggggacgaagcccaaggctgggggtcccgggttcggaggaggcggccaaggtgcaggccgaggctggcgagcggcttagggacgtggctcgcccgccaggaccagagcgcgcggaggggcttcggggaagtttataacacatcgctattgattcccg

SEQ ID NO: 247 - DNA, 401 bp, Homo sapiens
  source 1..401: mol_type = genomic DNA
cggagcgacagactagggagctccgcccgggatttgcccatcggcggaggcgccaggctcccgtttctccccatccctctcgctgccgtccaggtgcaccgcctgcctctcagcaggatggacgtgatggatggctgccagttctcaccttctgagtacttctacgacggctcctgcataccgtcccccgagggtgaatttggggacgagtttgtgccgcgagtggctgccttcggagcgcacaaagcagagctgcagggctcagatgaggacgagcacgtgcgagcgcctaccggccaccaccaggctggtcactgcctcatgtgggcctgcaaagcctgcaagaggaagtccaccaccatggatcggcggaaggcagccactatgcgcgagcggaggcg

SEQ ID NO: 248 - DNA, 370 bp, Homo sapiens
  source 1..370: mol_type = genomic DNA
cgcggtggagatcagccgggagagccttctgctgcaggacagcgactgcaaggcgtctccggagggccgcgaggcccacaaacacggcctggccgccctcagaagcacgagccgcaacgaatacatccactcaggcctgtactcgtccttcaccattaattccctgcagaacccaccagacgccttcaaggccatcaagacggagaagctggaggagccgcccgaagacagcccccccgtggaagaagtcaggactgtgatcaggtttgtgaccaataaaaccgacaagcacgtcaccaggccggtggtgtccctgccttccacgtcagaggctgcggcggcgtccgccttcctggcctcgtccgtctcg

SEQ ID NO: 249 - DNA, 423 bp, Homo sapiens
  source 1..423: mol_type = genomic DNA
cgcgcgaagcagagaacgagaggaaagtttgcggggttcgaatcgaaaatgtcgacatcttgctaatggtctgcaaacttccgccaattatgactgacctcccagactcggccccaggaggctcgtattaggcagggaggccgccgtaattctgggatcaaaagcgggaaggtgcgaactcctctttgtctctgcgtgcccggcgcgcccccctcccggtgggtgataaacccactctggcgccggccatgcgctgggtgattaatttgcgaacaaacaaaagcggcctggtggccactgcattcgggttaaacattggccagcgtgttccgaaggcttgtgctgggcctggcctccaggagaacccacgaggccagcgctccccggaccccggcattaggcgccagctgccggctatctgcg

SEQ ID NO: 250 - DNA, 1267 bp, Homo sapiens
  source 1..1267: mol_type = genomic DNA
cgcaataatgctgcgtcacgaaactgcgcattccaagagaaacttttctcaggcccagaattaagtcggagggatccgccgcgcagctcaatccctaggaagcatgaaagaatgtattgtaaagagtaaaatcacgcgcaatccatcccaatataaccgcagttgttcgtgggccttcttgcagagaaaggctgcccaaccttgacggggccatgggcgctgcgggagggcaaaagaccacagagatctgcgaagccaaagtaaacaacggggttggggcggcgagaatgatcaatagcgatgctccgaaaggactccgcgataggaatcgagcgggaaggattccctccatcctaacagtccatgggttaagaggccgaagcttccctaaataccacactcccgcgagagaagggactgagaacaacttctcaaactattctctctcgaaatcgctcccttcctcgaaaattccatctctgagactcggatgaggtccccaccccctccacccttgtcccgtgaccgtcgcccgctcagcctcccagccgagtccgcggggcctggggcgcccaccccgcccacccaagggaccgcgcaggggtgaactcccccgcgccccacctgcgccttccagacctcgggcgccccggcgttgcctcgagagctccctgcgcggccgccgcggcacggaccagctcccactcccttacactgggcgccgctgcgctcgccgggggccggtcccgaggttcccaaggcctcgcgcgcgcgcttgccgtggcaaccaagacgttccacgacgcgcgctctcgaacgcttcgcgtcacgcggccgcgcggccccgcccgtcggcctcgctcccgccacagagcccgcagcacgccgccgccgcagcctaggtcacgtgagtacccacgcgcgcgtcttgccagcggattcatcaccggcctgctcagactaggttctgcccactctgaccttctaaatggtacgtgggaggacgtccgtccccttcggacccaagagtcaccgtaacactctagaaggggagaaaaggagcgagggcggcaggcgacagagaacctcgcgagtcagcggccccgcgcagacccccccaggcacggtcccctgcggccacgtcggctgctcggcgcctgcgcaatctctttctctccagcgaaaccgaggcctccggagagcctagtagagagtgtgggcagtgagcgcttgtagccgctagagggagcgctgggcacagtgcacg

SEQ ID NO: 251 - DNA, 736 bp, Homo sapiens
  source 1..736: mol_type = genomic DNA
cgcggcatgttcgtaccagcgcgggcagaaacgggctcacgagcgccctcgaattcacacacactcaccgaaatacacatctcagcccgtggaagcgcattcacacacacccacgtacactcctccgcacctcattcagatgggtccacgcggagtcagggctgcgcttcccttggcatgggcgtgtgtgcggcgagggcgtgtgtgcgtgcgtgtgtgagagtgtgtgtagagaaaccctttaggcagaccgcagctacgttattctgcaagcatactgcgattcccgattccgtgtatgtaggaccacaaaaaaattaaatcgctgttaaacttttttttgtaaattctcttttccgaaggaaggcgctggaagagctactcccggtggttaaaggcccgggagacctggtggcgaatcctggggcgcgtccgctttggcggagcgctgggctccgctagccggactggttccccatctaactgatctaatcaatgactccgaagagccggggctcctggccccgcccccgccatcctcttctccgcctcccattggacgcgggccgagaaggcagggccttgctcctcgctcggattggctgacagggaatcagtatcaatgtttaagcggcggcgtgaggtgaatagagtcagtcagcaagagacgctggggagagagcagggatcgcgagcccggcggatgcggcagcggcggaaggcggctggggagacg

SEQ ID NO: 252 - DNA, 379 bp, Homo sapiens
  source 1..379: mol_type = genomic DNA
cgccgggctctgcgggggcatctcctggacgatgtacaccgggtgcccgtagtcgccgctgaccttctcgtagtgagggcagaagacgctgtccgcagtccttagcgggatgataatgtcactgggctctgagccgttgttgttgccgctgcgcttgggtgtggccagtgtgctgagcgacagcgtggtcgtgtgctgcggcgagtgcttcctgtgtctcctccggtacttcagcaagaggaccaccagcgtgatgatgatgacgatgaagatgatgcatcctgaagcaatccctgcaaataaggccacttcggaaccgaggatgttgttccccgaatgtccggcgctgttgccgtctgtgctagaacctgcagacgcg

SEQ ID NO: 253 - DNA, 875 bp, Homo sapiens
  source 1..875: mol_type = genomic DNA
cgctcccagccggcagccgctctgggcgtgcacccgccacgcgaagccagaggttggacgacgcagccggccaggcgcggctcccgggggaccccgaaaccgcctgtggctcaggccctacttggcaagaagagactggggagtgagaggaggggtcactcttgctctctcgcacaatagacttcccgcaggcgagtgcgaacggccttttgcttcctccttcttttccaggacccttagcgacaggagaggaagagaagcggcccggcggtttaattgccaatttcctgatgcagccacagggcccagcagcggctcagcctaggccaccgggtccaggcctctaggcaataggcccggcgccccctccaagcaccgtttcccgccgaccccgcagtcatcactgtgagctcgagagtatgggcagcctgtggtgccctgaggcgccgggcgctcggcgcatccgccgtgcccacagcgctgaggaccaactagggctgccctcggccaggggcactgtgcccgtttgagtggcactcggacctggcgaggggtggaagcccaggtagagctcgcgtctttaagagccacccgacagaactgcgcgctacatctgcgccagctgggctgggtgggaacagcgcagaaatactcccgtgccggggcgtacagggcaggtaggcccagcagagagtcttctcggaaagtttcccacaaaagaagggccgggcaggccgagcggcaggaaaagccggtgctcagcggcgaggcccgagaggtctggcatccggcggcctcccagctttgctcgggtctcccgccgcctctctccagagccgcccgtagctccgtggggcggagggcgcaatgccccg

SEQ ID NO: 254 - DNA, 221 bp, Homo sapiens
  source 1..221: mol_type = genomic DNA
cgattcctatcgaatcaacacggccattaaggacaaaaacgggcaggacacaaaagcaagcctggatggcgcaccctacaaacgagttctgaaacaaagcgagtgccagacgcgcgtgggaaagacgcgcgtgggaaagacgtgcatgggaaagtcgcgcgtgggaaagtcgcgcgtgggaaagtcgcgcgtgggaaagacgtgcgtgggaaagacgcgcg

SEQ ID NO: 255 - DNA, 1283 bp, Homo sapiens
  source 1..1283: mol_type = genomic DNA
cgtgacgtaagtccgccgcgggttcgccagcaccttgccatcccgcaccacgcccacgccaatcttattggcgctgccttcaaaacccagcaccgccggcatggcggaggctgggagaaaacgccgacaggactcctggcaatgtcaggagctgtggaggtcctcactagtccgcgctgggccgcagctttccggagcgcagaggaagctggccagcctgcagatagcactgggaaagacaccgcggaactcccgcgagcggagacccgccaaggcccctccagggacctgtcttcctaactgccagggacgccgagccaactctgtgccttacattcgtatccgttttcctatctctttcccgtggtccagcccagccttctccactgtttttttccctcttgcacatagttagaatcttaagtcagtgtcacacaatgtgctgtgcatctggcacaacgataaacagcccgagggagggttggggacctaagtgtcctagagaattagaggagggaggcgaggctaagcgtctccgtcacgtggtgtcagacagaccaatcacgcgcattcttcggccacgacaagcgcgcctctgatcacgtgaccaggtccgctacccacgtgggggctcagcgtgcacccttctttgtgctcgggttaggaggagctaggctgccatcgggccggtgcagatacggggttgctcttttgctcataagaggggcttcgctggcagtctgaacggcaagcttgagtcaggacccttaattaagatcctcaattggctggagggcagatctcgcgagtagggtacaaggcactatgaaatgatctagtttcgtgggtgaggggctgaagggcctatgatgcacggaggcggggaaaggatttagagataacgtggtttgaaaggcgggacctggtgcggggacgctcttgggaggagtcttctccccagccttagctggtttcatgatttctttgcgtctgtaggcaacgcggtaaaaatattgcttcggtgggtgacgcggtacagctgcccaagggcgttcgtaacgggaatgccgaagcgtgggaaaaagggagcggtggcggaagacggggatgagctcaggacaggtaagggaatgaaatcagcccttcttcctagaagctgcggcgggggtgtttgtcattcccttgatgtacggtaagtacgggccgactcatttttgcaggggtttgtgaagaagtcgcaggaaccgtaggctttcgttgggtctatagttaacgccggatcg

SEQ ID NO: 256 - DNA, 1167 bp, Homo sapiens
  source 1..1167: mol_type = genomic DNA
cgcctgttactagaggcgagaaccggagcccattggtcggaacacctcacaatggaccccagcggcgcgcaaaatccttatgattggtttgctggctgcctcgggagaccctgttgccaggatacttggcgttcccgacccgacccccgttccccattggctgtcagggcaaaagccgccatctaatgaggagcgaggtgcggtgccccgaagcgctcgcttcccgcggtgcgatctagtcctgcagtaggcggcccggggccacaccgcggccgcccaagccagtgcaaggcccaggggcctgacatcgctcccagcgctcgaggaccgaggcctgctgtggaggacaccgtgctccctcgggacctgctctggattccggcccggacgtccccttggagctctgcatctccaacctggaacccaacccagaagtctcaagtttgacgcatcacgtggcgtgcggatccactgagggtccacagagaggggcgcccatctcctgcgtctcagttatcctggtaattgtgtatctgcccattgttcgttgcctcattaacttggctttctaggtgcacccaccttgccaccagagaagtccaaatcctgacttctctccaaggtgttgggaattctgtgccctaaagaattccgactcagatccgaacggggatctggtggaatcgagggtgaaagaccagagggacaatgttctactatcccaacgtgcttcagcgccacaccggctgctttgccaccatctggtaagggcggggcccgttggcgcgcgatggcggacgctgcccgggatcccagcctgacagctccccctccatccccattctcccaccttccccacccacttcaggctggcggcgactcgcggcagccggttggtgaagcgcgaatacctgagggtgaatgtggtgaaaacctggtaaggcccagaaaagggaaggagggcctggtgcggggggtgagttaggggatggggtggccaagactgtgggcccactcctggacgcagcggtaatcagggcgcattgttccccagcgaggaaatcctcaattacgtgctggtacgagtgcaacccccgcagcccggcctgccgcggccccgcttctccctctatctctcagcccaacttcagatcggtgtgatccgcg

SEQ ID NO: 257 - DNA, 300 bp, Homo sapiens
  source 1..300: mol_type = genomic DNA
cgtagcgacgacgtggtccttgtaggaccaagcattcttggcattctgtcgagattgagattccagagcatcggatgaacgggacttatagctaaggcgtactcacccacccctcccttcgtctttttacgcacgcagcaactcgtcctcacgtgcataggggtagtggacagtgttgacttgaattgctgtccctcgttccaagcctttcctgtgaatgaacccgaaaacactcgacaggtcgtgaataatcgttttaatgagtgtgcaaagcgtgcgacggggtgcaccgagctgtcg

SEQ ID NO: 258 - DNA, 1492 bp, Homo sapiens
  source 1..1492: mol_type = genomic DNA
cgagaactccaagtccgacgagaaggggaaccagtccgagaacagcgaagacccggagcccgaccggaagaagtcgggcaacgcgtgtgacaacgacatgaactgcaacgacgacggccacagctccagtaacccggacagccgcgacagcgacgacagcttcgagcactcggactttgagaaccccaaggcgggcgaggacggcttcggtgctctgggcgcgatgcagatcaaggtggagcgctacgtggagagcgagtcggacctgcggctgcagaactgcgagtcactcacgtccgacagcgccaaggactcggacagcgcaggcgaggcgggcgcgcaggcctccagcaagcaccagaagcgcaagaaaaggcggaaacggcaaaagggcggcagcgccagccgccggcgcctgtccagcgcgtcgagcccaggcggcctggacgcgggcctggtggagcccccgcggctgctgtcctcccccaacagtgcctcggtgctcaagatcaagacggagatctcagaacccatcaatttcgacaatgacagcagcatctggaactacccgcccaaccgggagatctccaggaacgagtccccctacagcatgaccaagccccccagctctgagcacttcccgtccccgcagggcggcggcggtgggggtggcggtggcggggggctgcacgtggccattcccgactcggtcctcaccccgcccggcgccgacggcgcggccgcccgcaagactcagttcggcgcctcggccaccgcggccctggcccccgtcgcctccgacccgctgtcacccccgctctcggcgtccccgcgggacaagcaccccgggaacggcggcgggggcgggggcgggggcggcggcgcggggggcggcggccccagcgcgtccaactccttgctgtacactggggacctggaggcgctgcagaggttgcaggcgggcaacgtcgtgctcccgctggtgcacagggtgaccgggaccctggccgccaccagcacggccgcgcagagggtctacaccacgggcaccatccgctacgcgcccgccgaggtgaccctggccatgcagagcaacctgctgcccaacgcgcacgctgttaacttcgtggacgttaacagccccggctttggcctcgaccccaagacgcccatggagatgctctaccaccacgtgcaccggctcaacatgtcaggaccgttcggcggcgcagtgagcgcagctagcctgacgcagatgcccgccggcaacgtgttcaccacggccgagggactcttctccacgctgcccttccccgtctacagcaacggcatccacgcggcacagactctggagcgcaaggaggactgaggcgccgcccgtcctgggcccggccaggccccgcttggaggaggcatcgtcggcattttcgtttagacctttaattctagcactttgaattcgagcaggtcagcgtcttctctcgccacgacg

SEQ ID NO: 259 - DNA, 301 bp, Homo sapiens
  source 1..301: mol_type = genomic DNA
cgcccggccgctgccgatctcggagctcggaggccgagctccacaaatttgggtccaggcccgctttagagcccctgggcgggggcgcttctcagggcatcctgtgagggctttgaggcctccttggagtcgccagatgaactggtgccgcctgtgcgccttggccccatagcctcaacggcactctcactgcctgggctttggtctttgcccaggagcctgcccatggcctacgctccccgctcgcagcatggcagaccctgacgaggctgcccgccttggtccgggaatggaccgatcg

SEQ ID NO: 260 - DNA, 1761 bp, Homo sapiens
  source 1..1761: mol_type = genomic DNA
cggggcagggtacccgcgagggcaggcacgtggcggctcctgcggtgtcccgggttaaacttgcctgtgtttaagacgggtctgcgacagcttggggcggtccaggtcgcggggagggcgggctcctcaagtgggggatccgcggcagtgaggactgtagggtgcgcgggtactccgggtagccgccagcggaggaagcggctccgcccttcccggaagctgcccttgctacacccgtggggcctgctcccggcgcagtcccgcagtacctgagcggcactcggtcctttgaccacgcttggctgcctcagcttacacttcccagcaggatctggctgggagccccttcctcagatattcaccaggcaggtagaagcagagggagacgacggcgaaaggtggaaggagaggccgcggcctcgggcctcgggccccgggcccagacggcggcctctcgcggcgacagggtccttcctatttagcaggaccgcagggttggcagacagcagggcctgggccggcggggctctgcggtgcccattcgagtctctccagggcttccctctgtggctgtccctcgcctgccccctttccttgcctacccttccctggacggggttgttgtcaggtgacggcttcagcaacccagccatgcttggtgtggagtggttcgagttcctccgcgggcccccaccccccacccagataagaagcttaagtcctccttctgcaggggagaatcggatgcaatgaaactaagacctgtggtcgttaacgcggttcaaagccgaccccaactgggacacggctcagcttagaagtgaggaaggcctgggacgctggaaacaaggggtcacacgtgcgtgtgcctggccttcgcaggtggatgtgtgcgccgagctggggaaggggccagggagccgcgagactgcggtcccccgcgagagagaagtatgggcctggccagggttcgaactccgttccttcgctgccttccttttatttttcttcctcatcttccttcctctctcccttggttccatcattaaatctttaagggattaattcacaactctgtagtaggtaggggccctgggaccacttgacgggcctttgcaggcagttctgtacagagtcctaacccgtaacgggcctcaaccccggctgtgtgtgcgtgtgcttgtgtctgtgtgtctttgtgtgtaaaagcaacgcataaacatacccagggctttcacttattttattgaagcaaacaaacaaaaacagccactgggtttgtcttttcccctactcctgcctgagcgccgccgggtttctgagctgctccgggcgcggacctagctcacccgcagccggatgtcggagccggagctgctccccacccggcctcgcccgcgggcgacaaaaccgtccctctggccacgggagagcatcgccttgccaaaatcaactcgcattttcatgtattcaaatgagaaagactgagaagggcgatttcccctgctgggcagtcgacagcgttggaaggaagaagtgccccctagggcctcgcggaaacgggaacaaacctccctcgaggcccccggaaccgatatggcgaccccgcccagacctgggtccccgcggccgcgcgcgtctcccgccgttgtggcccccgagttactgacttttgttcacaccggtcagcgcctgttggaggacgtgctcagccccagccggtccctcctcatccagccgatccccctcctggggagcgcg

SEQ ID NO: 261 - DNA, 219 bp, Homo sapiens
  source 1..219: mol_type = genomic DNA
cgggcctcgaggatgcgagtgtgaggtcgacaagatatcgtggaagagagcgaacgagggaggcccccgttggaaacagtttctgtgttagaacaaacgccaccaaacgaccgacactgcgaggcctggagctacctgggctggcgccgcccgagccccgggctgcgagcccagaggctgggaagtgatgggacctgggggcccgagccggagagtgcg

SEQ ID NO: 262 - DNA, 2448 bp, Homo sapiens
  source 1..2448: mol_type = genomic DNA
cggcgggcgctgtcgagcacggggaggtgctgaaatagtcctggcgtgctgattcaagctttgattggcagagccacccggtgactgacagggggtctccatggcgcccgcgccgccaatccgcccaccccaatagcggagccagctcgcctgccggcgtgcctgagccgagccgagcccgaaccccaagccgcggagccagcacctcctccagtcggggtcgtccgctcccggccgttgagccaccgccgccacccggtagtgtgtcccgctgccccaatccgcctcatcaacaagcgcctggcacactcagccaggcccgcgggcatctgctgcgtgtcccgctccgggctcagtgccctcgccgccgccggcactgcctcgatgttccagctgcccatcttgaatttcagcccccagcaagtggccggggtatgtgagaccctggaagagagcggcgatgtggagcgcctgggtcgcttcctctggtcgctgcccgtggcccctgcggcctgcgaggccctcaacaagaatgagtcggtgctacgcgcacgagccatcgtggcctttcacggtggcaactaccgcgagctctatcatatcctggaaaaccacaagttcaccaaggagtcgcacgccaagctgcaggcgctgtggcttgaagcacactaccaggaggctgagaagctgcgtggaagacccctgggacctgtggacaagtaccgagtaaggaagaagttcccgctgccgcgcaccatttgggacggcgaacagaagacacactgcttcaaggagcgcacgcggcacctgctacgcgagtggtacctgcaggatccataccctaaccccagcaaaaaacgtgagctcgcccaggcaaccggactgacccctacgcaggtgggcaactggttcaaaaaccgccgacaaagggaccgagcggctgcagccaagaacaggtcggtacctagaggcctccgcgctttgagcgcaccggggaggaggcgggtggaggcacctctggcgcccttacccagtccctggcgactccaattcagcaggagttgggagcgcggtctgtcttgggttaagagccctgcgttctgggctcctggccgggagttcccttgccggctctgcttccccacccgctggctccccacgcctgcgggcagctgcagcagctggtcccggtcaccaaaccaaggcttcactgggacggagaggggaagagaaataaaaaattaaaatcctacaaacagttagggaccccaagacccaaagctaattcttgtcagcctgggcacaggctcctactattaatcgaagcctggcttattagcaatgtgtcggtttcatgttaattatcattttcaaagcccaggtatatccctccctaatgctttgaaaacagttttcaatggacttttgagaaatgggaagtcgagttttcctcttcccatgcgctgcctgccactcttgtctcaaaacagcaaactagtccgtgggccgaggcttttcgtttcccggagtgtggatctcgattagccaaacattttgcggaagagcccggcctcatcccccaggcccaaatgctccttacaatcctttttgcctttaggtcgggccgacccgatccaacgcgatcgcgggagcacttgctcaggcgtaagccccaggcagacgcaccgttagaaatggtatcccatgtccctgggaccgatctgtccttgtcacccacacttcgtttatttcctgacagtcctgtaaatctcccaaaagtgcacaacaaacagggaggacactgcaagcccagtatataaaagacctgggagctgcggcgctgagaaagggcgcgaatcatggtggggcacaacagtagggacccgcggaggggcggccgcggactcctgcccgacctctgtcgccttgccgagtaatcctcgccttaactgctggggtcttcggaagaacctctagccgccgggctggagggacgcaggaggtggtgggggcgggcgacgggcggctgtgttacgagctgtgacccgtgttccctttcttccccgtagactccagcagcaggtcctgtcacagggttccgggcgggcactacgggcggagggcgacggcacgccagaggtgctgggcgtcgccaccagcccggccgccagtctatccagcaaggcggccacttcagccatctccatcacgtccagcgacagcgagtgcgacatctgagttgcccatccaggatgctcagaagcagattccagtgtaaaaacgagaaaaacaaaatgaaagaggggaagaagatgagagacctgcaaatccagcgccacagaagccaggtgaccagggacccgcgggctcgggttgccgtttcccgccccaccccgcggccggcctggcttcactggcgccctttggccgcgaccacgggaaccagcg

SEQ ID NO: 263 - DNA, 685 bp, Homo sapiens
  source 1..685: mol_type = genomic DNA
cggctaggctgcgcctgcatcaggcgtggtgattggcaggcgcctacggcccacgcagagatggccgcgtgggggtcggagcgccaacttctcagctccccttctcccatgaccctcaccccccacaccatcttcagccgcccactgggagcttcctccacccctaccccccagtgccggcgaggccccctggcagatggaagctgctgactgatgggggctgccgagcgcgagggagctgagtgaggcccttgcgccctaggcggttgcaccccggacccctgcagctgtggacgccggagctgtgcgcagtcatcctgagccagcccgcggggcccgatcggaagggacgcctctcctaagagctggcctggagtcctggagcctccaagcgggcaagtaggggccgcaaaggctgggctgggcctccgctgaggccctcgggggcctctgggccctcatggcctggaggccgcgcggcccaagcccctagagcagaggccgcgctagcctgcaggggtcgacgcagggccggaggcagcggcttttccttcccactccgggttgaccctaaagacacgatttaacgtggcggcggcagctgcccacgctgtctggaggagcagggcgcctgggatgcggcggcgaaactggcccggaggggggcccaggcctcacgcgcccg

SEQ ID NO: 264 - DNA, 1592 bp, Homo sapiens
  source 1..1592: mol_type = genomic DNA
cggaggctccttccgtcccggcctcacctggatcccgggaaccgcccgctctgtcccgccaaggtgggctgaggaagacggcgccactcatcagggcaatggcggcgggcggaagagggatccccgtgcgcccccagcacccgggacgaatgccaccccggcgaagcgacccgaccggcgcgcacggtttcggggaggggaccacttccacattccccatgcccgagaaggtcacccacccgggagagcgcccaagggccagagaggaggcgactggtggaggaggggcggccccgtgcgcacaaaggggcccggggccggcaggctccccgcccttgaccttgggtctccgcctccatccatctccgccggctgccagggaagggggctggcccggggccagaggcgtgctcgctgggctgctgctgggctggggaccgacgggggcgggggggaaaggagccgggaaaaaagactccagggctgcagagtgctccgaggaagcctgcattccaccaccttcaggagccggtcccgaggcggctccgattcactgcatgccccccgccccccaaagcaaaagttgcacggatgcacgagggaaaccccttcccaaagctagcccttcgactcgcccaccccccgccttcctcggacccaggccgagtgggggaggggagtgtcaggggaggaagaaaggagcgcgccttcccagcgaggccagacgcgggctgggtcagctgcgcctccgcccgcccctcgccgcccggctcaggcagctgctccattaagcccccgaattatgcatggaccttgagccctctcggccctcccgccccaccaaggcacaatcaacgtgcgcagcgggcgcgggtcaaagagagagggaaggaggtggacgatgggcgcaaggccgtgggcgaatcagaggccggcggccaggcgggggccaggcgcgcccgcaggcctgggaacagtgccgaggagcctcccgacccggccagggccagccgggaaaggggcaccgctcgcaggggaggcccccgcgcagttttgaagttgccaaagtgtccgcgccgtgccgggtgcggggtccgggagatgcagccgcccccgcggagctctgcgcccctggcctcggggaaggccattgtgttctcctgtgtggcaatgaagggggctatggaccccaaccattgagcccttggagggaggggcctgaagacccctcgggcagggcgggggaggctgtcttgcgggcggcggagaggatgggtatgatttgctcgggataggcaccgtcgcggggaggggaggaggccaagcttgaggaggggctgctggagatggaaggaagtgagaggagacttgcaagttgcaaacgtcggggaaaccggaaaattgcagtagcggggaggcaggtggcgcccagaggttcgaaagcagaattgggaggacttcgatgtccacctcgcagacagtgacccaggccccagagggctccgcagcctggagcctcgcgtcccgcctccccgcaacacgctgttttcagcgttccagaccacagcgctgcagtctacgctgccctgacg

SEQ ID NO: 265 - DNA, 3241 bp, Homo sapiens
  source 1..3241: mol_type = genomic DNA
cgccaaaagaatgagcgcgggagggggtcacagcagccagttcggccaaacatcgacgctgctccggcacgctccagcccagcgcctgggatgtgtcagaggtacctgcgaggaacatttgcgtgcgattccacgcacgcgttgcatccctcttcccaaacccggatcgctgaactgagaggtggcgcagagtgtctcaccaggtgcggggatttctctccgcctcaggccgtacgagcttccctcgctgtccctgagcgcaaatgagcctcttcagtcgccgaggcccggccgcttagctctcggagccttccacagcccaacaccctgagccctggagtaggcaatttgctcccacagccctgtgctggcccttccaggcacagggagtcccccttctaggggtaggggcgggcgggatgaagcgcacccagccctctctcctacccttcctcctttggtcccttcggctttcgtgtaccttatctcccgcgcgcccagctccttggccggctcacctgctaagcggagcgcggacatgcgctggaactccggctcctgcagccacttccacatcctccggaaggtctcccggccggatttgagtttgctccagggtttggggttgcgcagcaggtccgagagggtcccctgggagcggcagagcaccctctgcgcgaagatggcctgtgggatgctgtagcgcttgagctcggtggtgatacgctgcgccacctctttggtattgatctcttccatctgccctgaattacttccattgctgacctgcgcgccggtcaccgaagggttgggctcccgggctgtgcccaggagttgcccgtggccctgggcgttcaggtgggcgtggggatggtgcggaggaaggccgttgatgggcaccatgccggccgaggtgggcgtgaggtgctgctccccgtggcggccgagcatggccgggtggtgggcttcgaagccgttgggggtgagcatcttgtcggtgggcatggcggcccccgggtgggcatagtgggggagcccttgctgggagttgtggatgctgcccagaccggagctggagaggggcgagaggctctggcccatgccggccacgtccttgtggtagggggtatagaggttattcatggaggccagcccgcgctcatcccgcatgagcgtgaagctaccgctcacgttgcccgccaggcgctggtggtggtgcgggtggtggtggtgatggtggtggtggtgatggtgggggaacttgtccgagactgtggagatgggaggcagcggctgcagaggggtcaaggtggtgtaggtggtgggcatgctcatacctgggggagtctcgcaggccatggtcatggtgggatgcagggggccggccaggctgtgctcaggggcccggtggtggtggtggtaatctccgccgccgctgccgccgtccagcagggacgccatgcccatggagcgcgggtgcgcggggggcaggtggctgccgcggtgcgccacggagctgcgcgcgtgggggctgccgcccagcaggtcggcaggggcgggcaccggctcatggctcaccccgtgcagctcgccgatcgcttccatggtcagctgcgcgttcatcgtgatccgggcgagcaggcggcggacacaacatcgatgtggccaggcagaggcggcgaggggcgcacggagtccggtcttcacatcggctgctggcgactgttgccttccttcctctcactgtggggctctgtctctctctctctctctctccgtgtgtgtgtgtccgtgtgtgcgtgtgcgtgtgtgtgtgtgtgtgtgtctcgccttccctcttaccccccaccttcccctctgcgtcctcggcttttttttttttaatattaatttccaaagaggatccgcgccgttgggagagcgcagtgccccagcccgcccgcctcggccacctctcgcccctctctttcttaaaattctgaggtctccggctcccctgccgcggcccgcgcgcctgccgcgtctgctgcctgcccgccccgctggccagcttgagccatggctctgttactgttacagactctgtggccgcggttcggtagccgccgccgccgccgccgcagcggcccgccctcacgcccgccaggcgcagcgcgcatgcgcgaggcccgcccccgccccctaggtcgcggcgcgccaggcccttggcgtcccggtacaaatgaaggagggggcccagcgccttccctgcggcgctggatggccagggagctgcgggcacgtgcgaaagattggcgcagagcgcgacctggggccgccgctgcaatcccaggagactcgcgcctggctcgctcgcctcccttgcttgagtgggctctgtcctcccagcccggggacgctcgtgtcgggcttctagcggctggagtgctgtgcttggagacatcgcccctctctctccagttgctgcttcccggtgcagcttgcccggggagctggggactgtgtcatcaccccttcggctctagcccactaagctttatttcccggggggctgctgggagtgcgctttcccacccttgaattcacggcctattgggggatgggggttggggtggggtggggtgcagattgcttaaagggctgggtccgtttggcggccgttgacccggcacccttcgtcctcccagatacatacttgcccaccctttctcatccatgccctggggagaggagtaatcagtgccaagaatcccagttcgggccatacctcactgtcccccgccgcctggctccttctcccgctcagctcataattaaggctgtgcgtccgcttcgccgatgactcggcctgtggaggggggcgagggagagcggagggagtgccctggagggtacccatgttaacttccgggtgctggtggggccgtggaggctcgggccgtccctgcggttactcccaaggccctcctgctaaagcacccggaggcggttgctttccagaagtactgacgcagacagggtggacgccggcgcgcgggtctccgcttggcccctagggacgcccttttcccggcgtccccgagagacgcctccagatttgaaaatcaattcagcttcgggagtaatttcgcccttcccacagtcacgctccaatctggaatcgaacctggtctttgggcctggtggggacgtgtgcggaggcccccagtttgagacgtacacccggccgccacatgccgcg

SEQ ID NO: 266 - DNA, 2385 bp, Homo sapiens
  source 1..2385: mol_type = genomic DNA
cgcgcggctgctccatctgtgaacgtgagtggtcgcctggctccctcttccgcggcggccgcttcctcataccttcacacggcgcacaccggccggcgcgtccagctgcctgcccagtggccgaggctcttccccctcgcccagtcttgagctggaagtgattcctattggccaaggtgtccatgtaaataggtgtgaaagaaaccagagctggccgggctctcccttctcgctcagtcccctccctctgcagcccccgctccccctcctcttcctcctcctcccaaggcgattgtcatatgatagctaagaagtggcacattaatgaagcgccgctacaggggtcttttctgctcctgtcaccgcttaaaactatcagatggttcgagggaggacatggaggcagccacctagctcagcggagacgcggagcccacagcagcgccctccggagccctaacacgtcgctgccaccatccgcgccgggactccgcagccgagctcggccgcccgcaggacgctccaggagcgtcgcggaccgggcggcacgggacgctgcggggctgagctcaagagcccaggttcgcgccgagtccaaccggacccggacgctgcgcgcggagtgcgcgtcgagtgcgcgccgagagagaagcggcgcgcagcggcgtcctcccggatgcggacgcgcaacttgaagcaactttaaggtgagcagctctctgttccgtccctgccccctattctggccccagtaccgacttacttcccggctatcctcgcgccgttcgccggcttcccctcccgcgcccactaagcccgcaaagttgctggcgaaagagtccgggcgctggctgatcgagcgccgcaggccccacccccgacccccgaagtctgttactcggtctggctgaccccgccggtgtctctgtgcatccatgctacctttccctattacccacccccttcccagatccgagcagtccgccggcccgcgcggacccagagcaagaagagggcgaggaagaagatgcctcggcccggccgcaacacgtacagcgaccagaagccgccctactcgtacatctcgctgaccgctatggccatccagagctctcccgagaagatgctgccgctgagcgagatctacaagttcatcatggaccgcttcccctactacagggagaacacgcagcgctggcagaacagtctgcgccacaacctctccttcaacgactgcttcatcaagatcccgcggcggccggaccagccaggcaagggcagcttctgggcgctgcacccaagctgcggggacatgttcgagaacggcagcttcctgcggcgccgcaagcgcttcaaggtgcttaagtccgaccacctggcgcccagcaagccagccgacgcggcgcagtacctgcagcagcaggccaagctgcggctcagcgcgctggcggcctcgggcacgcacctgccacagatgcccgccgccgcctacaacttgggcggcgtggcgcagccctcgggcttcaagcaccccttcgccatcgagaacatcatcgcgcgggaatacaagatgcctggggggctggccttctccgccatgcagccggtgcccgctgcctacccgctccccaaccagttgactaccatgggcagctcgctgggcaccggctggccacacgtgtatggctccgccggcatgatcgactcggccacccccatctccatggcgagtggcgactacagcgcctacggcgtgccgttgaagccgctgtgccacgcggcgggccaaacgctgcccgccatccccgtgcccattaagcccacgccggccgccgtgcccgcgctgcctgcgctgccagcgcccatccccaccttgctctcgaactcgccgccctcgctcagccccacgtcctcgcaaacagccaccagccaaagcagccccgccacccccagcgaaacgctcaccagcccggcctccgccttgcactcggtggcggtgcactgacccgcaggagcccacgccccctctcgttctcctccccaccacctcactcgccttccctggctcccagtcctgccggcccccacctgggaccgccaccctaacttgttcatttcaccttcggccaacccgccttgccccaagagaactttgttttggacccaggagaccaaacacaaacttgcagatgggccgagaggcgcgtgggagttgtcctcgcccccacatcaaggaaggccggggccacctgagccgaaccatcccctccctgaggccccgaaaccccctcctatttgaccggcgggggaaaccctgtcaccccctcttcgccgagaaaagcgcgtcttccctccgcccagatcggcggagccccgaactctgcg

SEQ ID NO: 267 - DNA, 264 bp, Homo sapiens
  source 1..264: mol_type = genomic DNA
cgcgcccctctggcctccatcactgtcgtgccggtcgtggggacagatgggagggaattacggtatttacagctgctcttcgccggctcttgctcccccgcgtgtcgacaaccgaaactgcagcgaggcccagaggcctctgcccactcccctcggagttccaggaggacgctaagcgcgagaagccaggctcagggaaactgaacgcccatacgctcctagtccctctcacctggatcctctgcgtcaggttacgtgcttgcg

SEQ ID NO: 268 - DNA, 7090 bp, Homo sapiens
  source 1..7090: mol_type = genomic DNA
cgggacgcaggtgcagggtgctggacgcaggtgtgcggccgcttacctggggcgtgcaggccttgggctgcggggaggcggcgggcgccgcgcgtggcccgcgggcgggcggacccggggagggcgctagcgactgcgcgggcggtgggtgtggcgccccggctctcagctgtcaggcgcgcctgccccgccactcgggctgtgaccgcgccgtccccgccgccgcgccatgcgctccaggcttcggcctcttcttccggccagtccccggcctcggggtcaacggctcagggttcgcatctccgcgcccggggtcccggcgccccgtgcccgctgagagcgggaggggccgcgggtgggagcgcggagaaccagggagcacggctgtgcgcgcgcgcggggaggggccgcgccgtcacccggagagcccggcggatcccggactcccgcccgccgctcctcccgccgcgcacctccgctcaccgctcgcccggctccagccgccgccgcactgtgagtacccacgtccggtagcccgccgcccccgcgctctgctcctgcagcttttttttcgtttagttctgatttgggctcctgtggctgttgtttttatgattatacctaaagacaccaccgcctcgctcccaccactgccagtctcccgcttcgctgccctccccaagctcgtccatctagctccgggagccgacgggggttcctcaagctgctctggctgctgctccttgtcgctgcatatggctcttcgcctgtgctttccgctttggcccccgaaattccgttcctttcctagggacccccttcttctgactccgggcccggacccccctcctctcactcagggaaaaccgcgcagggacccaggaccctcaactggggtacgctctcggctcgggcgctgtcttcactgtatgcgggccaggcatagttgccgatttcgagaaaccgagaagctgtcatttcccagcgctgcgcgccctccagccacctccaggagggcgcgaggcttgtcagccggggagttaggacagtgtttttgcctcctacaatgatataaaggacttcaacgcgaaataagggtcaacttattttagttcgatcctcggagataaagatgaaacagacagataatacataaatagcttgatacttgaagaaggtacaaaacagatgagagaaagaggaacgagccggcagagacagctcagggacagataagatcgacaaatgagggtctcctagacaaatagagaagaagggagagagacagataaacagaagattccagtctccttgctgaaggcttttgaactgggatcccctgtctggagctgcccctctgaaaacggagttacattcagggccgactgcaggggaggagccctcagggttgggctgccctggccgcaggagcgcctgcagccttgggccggatgggagcctggcttgtctgaggacatgtggctgggtcaggcgacctagagggccccaaacatcagtccccaagtacccccgctgctgaggtctgcctgcacagctccctccctgcctcagccagaaagcaaagtgcaaggaccagcagcaacttgtgtgcctggagccttgcaatggctacgaagatggctgagattccttctagcccatatgagccgggacagcgaggcatgaaggatactcagagaggtgatgcccctcaaagaggaagcccagagccccgaatcctgcagttggcccgagtgggaaacgtatatctgaaggaggaaagctcaaggagtgggcgactggaagctaagaagtggatcccaacaaagttccttgagaacctctctgtcccacacgttgacctcggtggaaaccagctttggtctggaatacagctaaaaatgcagaaaatgtggttagaactgcaggatcctggtctcactaatagctggaaagaaaacggtttatatatccagccgaaatctttctgctcaacgaacgcgggcgccagccgtgagaggagaagctcgggtttgtggaaactggcaatgttgtcccctgaagccggctcgggcaccaagctgggtgcggtgggggccgcgctaggtccgggtgcgcaccgagcgctttggagagggtcaggttcagcaccgcgaacagcggtagcctccgcctctctccttccctggcagacacggttccctcctccttctcctcacccccctccttctcttcacccccctctaccccctgctgtctggagatgggcagtgaccgcccctcccccctccccacccctccttgcactgcccccgcccgcccgcgtgcatgcgcgactgagcagaggggcggccgatccccgaagtccgggcttcaggacccgggccggcagcaccggctgcgagggttgccgaaggcgcacggatctgggcgctgaaaaagccaggatttggcaatggctttgctgtgtggccttgggcaagtcactctgcgtatctgggtttcacttccttcccaatccgaaaacgggattgggttccttgcagcccgggcattcctgaggtctcctttaccccttctcctcccccaagccccgggggctgttgttaacggcgccaacatgggtctgagtaacaaaagacgcctgtccagggggtgaatgagtttggactccccactgccaagtatcccccgcgcgcccaactcggagcgccctgctgggcgggcccgagcctcggcggcggcgctgaaaatggctcatctgctctctgctttctctatttcgcaggagcggcggcatggaggctctcaccactcagctggggccggggcgcgagggcagttcctcgcccaactccaagcaggagctgcagccgtactcgggctccagcgctctcaaacccaaccaggtgggcgagacgtcgctgtacggggtgcccattgtgtcgctggtcatcgacggccaggagcgcctatgcctggcgcagatctccaacaccctcctcaagaactacagctataatgagatccacaaccgccgcgtggccctgggcatcacgtgcgtgcagtgcacgccggtacagctggagattctgcgtcgggccggggccatgcccatctcgtcgcgccgctgcggcatgatcactaagcgagaggccgaacgcctgtgcaagtcgttcctgggcgagcacaaaccacccaagctgcccgagaacttcgccttcgatgtggtgcacgagtgcgcgtggggctcgcgtggtagcttcatccctgcgcgttacaacagctctcgtgccaagtgcatcaagtgcggctactgcagcatgtacttctcgcccaacaagttcatcttccactcgcaccgaacacccgacgccaagtacacgcagcccgatgccgccaacttcaactcctggcgtcgtcacctcaaactcagtgacaagtcggccacagacgaactgagccatgcttgggaggacgtcaaggccatgtttaatggcggcacgcgcaagcggaccttctccctacaaggaggcggcggaggcggtgccaatggcgggtcgggtgggcaggggaagggtggtgctggcggcggtggcggcggtggcccagggtgcggtgcagagatggccccaggcccgccgccccacaaaagcctgcgctgtggcgaagatgaggctgccgggcctccggggccacctccaccccacccgcagcgcggacttggcctggcgactggagctagtggcccggcgggcccaggagggcccggtggcggcgccggcgtacgaagctacccggtgatcccggtgcccagcaaaggctttgggctcctgcaaaagctgcccccaccacttttcccccatccttacggcttccctacggccttcggcctatgccccaaaaaggacgacccggttttaggcgcgggcgagccaaagggcggtcctggcactgggagcggcggcggcggcgcggggacaggcgggggtgcggggggcccgggagccagccacttgcccccgggggcaggggcgggcccgggcggcggcgccatgttctgggggcatcaaccctccggggcagccaaggacgcagcggcagtggctgcagcggccgccgccgccactgtgtacccgacgtttcccatgttctggccagcagcaggcagcctcccggtaccgtcctaccccgctgctcagagccaagccaaggccgtggcggcagccgtggcggcggcagcggcggcggcagcggcagctgctggcagcggtgccccagagcccctggacggtgccgagccagccaaagagagtggcctcggcgcggaggagcgctgcccgagcgctctgtcccgcgggcccctggacgaagacggcacggacgaggcgctgccaccgcccctggccccgttgcccccgccgcccccgccgcccgcacgcaaaggctcctacgtgtcggccttccggccggtggtcaaggacaccgagagcatcgctaagctctacgggagcgcccgggaggcgtacggcgcggggcctgctcgggggccgggacccggcgctgggagcggcggctacgtgagcccggactttctgagcgagggcagctccagctacaattccgcctcgcccgacgtggacaccgcggacgagcccgaggtggacgtggaatccaaccgcttccccgacgacgaggacgcccaagaggagaccgagcccagcgcacccagcgcagggggcggcccagacggtgaacagcccactggacccccttccgccacctcctctggcgcggacggtcccgcaaactctcccgacggcggcagcccccgcccccggcgccgcctcgggccacccccagctggccggcccgcatttggggacttggcagccgaagacttggtgcggagacctgagaggagcccgccaagcggcggcggcggctacgagctgcgagagccttgcgggcccctaggaggccccgcgccggccaaggtgagccccgcgcccgccccgccagtggcgccttcccacctaccctgtgcgcggcagggctgcggggccgggcggagaacgggaattttgttccgcgcaggcgtgggagcagcaggcctcggccactctccgcaggcccagccttgggcgcgctgcgaaactgggcccacccgcaccctccagtgagggtttcgaagggaagggaagctgggggcggcgggaggactggggaacttgtgtgctttgtttttgttgatgggaatggggaggagaccggctgagactgccagacacagagacagaaaaggggtgtggagaaagaaagaggaggtaagaaaggaacccagagccagagaaaagcgctcgcccgggtgggcatcagagccccaaggaagaggagaagagaggacagtgtggcctctccctgagtcgggccccagcagtaccgggtccccggaatctcccaccaccccaactcgcagttttccgagtctcttcgcactcccgccttgggaaccacagggttcgttgctgggaagctgtctcgggaccgcgtgcggagcgggaggggggctctcaagctggcagggccggggcttgaggggcaggatgcagccaggctggagtcgttggccattttgaaagtgagtgcgcatacctgtacattcggttcattaaaacacgaatcattaaccggatgcgaaaggcgtcatttatgccgcggacggggcactggcgggagggaaaatgcctaaaaggaggcgggtgcagagggtggaggacactttgaacgcccctcggcttgggagccgctttgtgtttagagtaattttcccgagggcgctaacaattaccctgaatctacaagggggcaagggtgcgccgcgtgcgtcactgtgcccgctcaagtccagcgggcgcaccaacctttgccactccgtcggctttccctcttggccgcggggtagggctgggcgtctttgggccgccgcagggcgccaccctaatcgcctgtcatttctcggccgtcgcaggtgttcgcgcccgagagggatgagcacgtgaagagcgcggcggtggcgctggggcccgcggcctcctacgtctgcacccccgaggcccacggtaacgcctgtcgcggccgctggccactgtaatgggggaaccgcagacagagggccggggtcaaggaaggcctgcgaaggaagacaaggagagagacgcagagaaagagggagagggaaagaaaaataatgaaaacaaggaagctaagctagtactcccaacaacttccaatctttagttacttaaaaacaacagtgacaacgccctctccaccacacgcacacacatgctgcagtacacccagacacggcgttaaacccttaacaaaatcacgcccgacacggccttcaaatcctctcatttattcagcaagtgtgtttggccgcggggtcccctttggctaggccggatcggggcggaggatgggggcacatggccgcgcgcttgggtaccccagaagcgccagggtagctgaggcccatcgcggggtggtgggggccgccctgcacttgcgcgccttaccaggttcctcacagaggaggggttgggggcagcggaaaatcgggcaggtcgaggcagccgaaccccggacgatgtccccccacccaccccgaaggtcgcagcctgggccgcgttctcagcaggagtcgggcggacagacccggcggccacgcgcgctcgcggtgccccagtatctgcgcgcgatgtaggtcgctcgtccctggtgggctcggctgctcgcctagctcttttccaaggggctggggcgggggcctgaagcccggagatgagcagaccaggcgcgggggtggggggtgcccggcccaaagcctcagggcgcgctcctgggcgcacagccgggaaccagaggcggccacgacgcttttaattagggctggggcgccccgaccgcggcagctccggcagtatccgggtcacggtttctttgcctctcttgaaattcggtttgttgccttcaagagccagataaggaagacaatcactcgcccgccgatgatttggaaacg

SEQ ID NO: 269 - DNA, 3174 bp, Homo sapiens
  source 1..3174: mol_type = genomic DNA
cgggaagtaaagcctcgggactttggttaagcgcgccccactggcgtatcgcgccgcgggactgcagagccgtgagcagcggtcgccttggtgcagggttccagccacatcttcctcgcccagccagcaccctctgccgttgacccgcctctgcgcccggcaagcggcttccagcagggggcgcgcgcggaccaggcttgggccctagaacagcgcggatggagtcgctggagcaagtccccagatccaaccggtttcaaccctccccaccctcccgacgctccgggttcgcgacgttgaagttaagggtcgatccgcagaaagcggccaggggctccagctctccattcctgggtctgtctggggtcggctccagcctggttagaagccttagtctggattcggcagattctgaatctgggaccctctgcgctagcggcttggaaccttgtcaccctcccctcccccacccctacttccacacacctgattagttgtctgtttctttaatgatcaaagacgtgggcggcggcgggatgaggtcttggttcccggctccacagccctccctaactgtcattattaacgttataaacattagacccgcttctgcgcgccggacggcgccggacgaggtgcgcgcagtcttctagctgagctcggaggcagatccagaagtcgcggctcccaccccaggcctcggcggactctgcctggggcgactcgggctccagccctgcccgggcgggcactgggctctccagggtcgaaggcaggggtaaggggcgtctttcccccagggcagcctccgggaacaaaagcatttgctgtagagtgagctagagcctccgggcccgcgggagtcagctcccgcccaggggtggtcaccgcgtccttaaccaccccaggagccccgtctccctgccgaactccttggcttctgcaaccctgtcaagacagcaaggaaagggggtcttccctggtcctcgggccccgaagtttcggggttgcttataggacgggttcctgcagtccagggaagctctgggcagatagcgagcccattctcccttccattacccagattctgcctccctgcggaaggcaaaaaagaaagaaagaaaataggtaaaaaccggcggagggccttgagcctccccgcctggcgcccctcactcagtcccgaaaagtcccctggacacgccatgccggaccggactcagctcccgctgctgggcccctgcctccaaatatccttcccaggcacaggctcccaagaccgccccctcagggttcccaacacccggacaactgcccaagacgccgctccccgcccccacccccaccttgttcggcagacaaagaagggtgtgctggccccgccgtctgcctccttctcccgaccccacaaggcctagaaacctcagggactcaccccgggctagggacccaatcctggctgtcccaccacaggatccccggcagggacgggtcacagtgctctcacccctcgaccattttcgaagacaccttccctgaaaggcgccttgcgccctccccatgggtcggcggggggggactccaggcccgagcaggcggtgtgaagttctgtgttctgaactggggctgagcaagatgcgatggtctcagcccgctgggccgcccgtagcgacggcaggagtaggggagagggagggacgcttggagtgtgagcgcaccagtctgttcatatttaatttacaaagcagcctcggaaccccgggccgggtggtctctttagacgctgcgctcttagcctgtctctcttccccaccccctcccctagctcattaagatgctcaacactcaaatcggggtattgatctccacggaagccccaaaccctcgccatcgagagacccccatggcccggggtgatggctgtggggcttggtgctcccagagagctcagtggctacagaatgggtggggattctgcgtgtctcccggagcctgaacccctttcctggttatggccggtagctgtctccagggctaacgtgggcagcgcaggggggcggaaaccgggttttagccaaatgcctcgacatcgccgcgcctccgcctcctcgtcgctgaaagaaatgtcggggtttcatcagagctagggagcgacagtcgggaacagcgagtctgccgaagccggctgttgtgtgagggtgtgagacggcggggcggtgaggggccaccgcggcttgggggatagtgcgtgtggggttgaccgtgtgtctgcttgagaggctgtgaagatatggggggcagatatgggagaaatgctcgggcctgaagtccccagcccaccgtgctcaagagtagcggacgttttgccaccatccttgtctgtgctactgtctgctgcagcttccgtgccccgttctcctggagcaggcaagacctggagtgaggtgcttgggtgcgctcgagagagcttccccctgctccacctgtcccgcggtgcgcgcaggccaacgcgtcgggcagtgggcttcaagcgctggtttagccacaaaagaccagaagtaaagagttccggcttaagaggctgggcagggctgcggtgggctggggaggggggtgtcccttcccagcacgccctgcagggctgtgcgttctggtgtcgggttagactagcaggcggggcgggggggttggggcggcggggcgggggagactagggcttatatcagcccagatccaggcaaaaatggtagggagggtgcggcgctctgctaacactatcaattatgcatcatgttgaacgtggcttcggggaggaggcggctagcagcggggggtgcgggagggaagggtccgcgcgagctcggctgcgcgcagctcagcgggtcccgctcgaagtctgtcggtgccaccgcctgcatttgcaaaaagagtttaaaggcaaagacacgccttccccccccacttcagccgcgcgcctttccttcccccaaattcctcaaagatggtttgtctcacgtgttgcagggcgtaaaagcggcttgcattcaattagcagcgaagctcgcgggcgctggcgggacaggcgcgtgagggtgagttcgcgtgaatgtgtgtatgcgtgtgcgagaggagaacggtaagtgtcccgggtgcaggtgtgcccgtgaaaatgcg

SEQ ID NO: 270 - DNA, 315 bp, Homo sapiens
  source 1..315: mol_type = genomic DNA
cggagctcaaaagccggacgctactggacgaggcttaaagcgctcgccccagcgcggggccggtgaggaacgttcaccgtggaccatggtcgaataatgttagcgtgcactcggggcctcgccctagccttctgcaaagcctcggcctgcgccctcggctgcatccgtttcgacgccaaccaacctcatgctcttagtagaacggcagtagcgaccgcgccctcaggcccacaagcccgaaggtctgggcagggtctggaatggcggtggagacactttatccgcaggcgtttcacctcgcgccccacccaggcg

SEQ ID NO: 271 - DNA, 1171 bp, Homo sapiens
  source 1..1171: mol_type = genomic DNA
cggcatcttggccgggttgtaggagcgagggagaacgcgactgtaacgcgggcgttgagcacctagagttccccttcctcttcactgagagctgcggcggaggctttagaccgaggagcgagatctaaggttaaagaaagggcctctgagcccagtgttggccagagaccccacaggcgggccgggttcgccctttgccttcggccgcgccccacccaccagggacagagtggccgcggccgagccgggtccacccgccgcagcttagctccgctccgccgccctccccgtccgtcctccccaggctcccgtcgccctcgcccaccctggggccccctccccagatgggagcccctccgacccctgcgaggcccgctggggacccacttgaccgcgagatcgtagtgggagctcggccaaaagaattcttcgctttccctggccctcagtttacccatctgggaagaggggcgtcgcggccgcagaagtccagctccgcacgcgccaagcgctgggtctgcagctccctcggttcccgcgcacccaactgccccacgacccctacccgcgcccgcagcccccgccctcccgccccacttgcctgggccgccgggcgctggaacctggaccctggaccctggcgggttccgagctgcgccgccgccgtccctgcccctccagcactggactcctcctttcccgtctttttaaagggaagtaaccggacccgaccactactccacccgattcacccctagcgggccgcccgcccgcagctccgcccgggctcctcccccgtccgcccgcggcttcccgggagccccccagggctcctccgctcctccgacggcccggccttccccccggggcctgggatgcaccagggctcctgctgcccccgacggggtccctgtcgccgcagcccgcgggacccgccggccacatctggacggcatctgccgcgggggaggggctcagagcctcgccctgagtgtcccgcagggagccttgtgcatccggggcaggaagccagcgcatcccgtgcagacgggtggtgcagataacggacccgccacagcacgcaattcaactttcttggtacctgggactcgtagttcagaaggcgcagtagcacgaccttgcgaaaaagcaccggagtcgggaacacgcg

SEQ ID NO: 272 - DNA, 257 bp, Homo sapiens
  source 1..257: mol_type = genomic DNA
cgccacccgcacgacggacgtcggcaccacacgagctgcccttggcaaaacgcgttgtcggctgctgggatggatttcaggttaaagcagagtcaccaggccgaccactgcttaccttgttagcatctagaaccttcttcagctcctcgtggctctgctgggtgataagcacggtctctgcggaggtgatgcagccgctgcacgccaggcagtcgtttagcgagaccttggccttctccagcctccgggtcccgccg

SEQ ID NO: 273 - DNA, 895 bp, Homo sapiens
  source 1..895: mol_type = genomic DNA
cgggtccactcctcactcgcgaagtccctatctatctctgtgggtcccggaggcacagggctgcaagggcgcccgctagagctgctccacggcttaccggattgcatgcggagaagaaacgggcccgggtttcaacagaggccccgtgctccgtgaatttctaactcgaaccagccgctgggataaatccgagaaacgttgtttattgtgctattattaactttcccttctccccacgcccctagaggaaataaaattcattcctgaatattgggccaggaaaacgtttctctgaaaggaaccacaactgcaggggaccacccggctgcagctcccagctcccggcccttcgccctaggctgcccactcgctccggctcccgacctccccgctgccgaagcaactgggtcagcctttcagacttaaaacgaagtcgtttatttcatttttgctttcacattatgttccgatacaatcaaaactcctggacgaacctcttgaataaagtcctgtgaatgagcggaatcataggtcctgggcgcccgagtatgcggcgggtggactccggattcagaggcggtgtataaaacatacgctgcttcgggaactgcgagggggacgcggtgggagacatccggggacagaaggcctgaggagtctgccgctccaggcagggtcgtttgggtcctcaaactgggactattcctgatgcatttggaatgcaaagaaaaaaatcaaacttcgctcctgggccggcggccgcgcctggcctagcaagatttcggccacatttcttacgtttccgccaaagctgtgaacacaggcgaatgcagtgctggtccccggcctggcgcaggaaggagcgcaaggccggctttgcaaggaagccctcgcg

SEQ ID NO: 274 - DNA, 2545 bp, Homo sapiens
  source 1..2545: mol_type = genomic DNA
cgcgaaggctcggggctgcgagccgaatgcccgccgagctgcctgggaacgggaaccgcgcacccgggaacgccaggcgtttctcttcatccaggaacggcgcggagagcctccggctggggagctaaaccgctggggactccgcggctgctgcctgagtcgctgtccgctgcccgcatcccttccgccctgggcctctgcacggtctgcggttttctgtgcgcacttggtcttcagtactagcacccaattacgtctgggtttttcttctttacagagctgggtttcggtggccaccagcttttctggtgtttagtgactctgagtttggaggtggcctaccaggcaaacggggattcagggcatttaggaaacgtcttccgcgcttaatcccagaagtggttgcgtgtccgtacgatcccaagttcctcccggagcctcttctttgctgcgtctcgggccctccagcaggcgaagcctcttagacgcgctggggaaacttccggcgtgttcggggctcagggtttcttctcaggcatggattggggcgcagaagttgcgcgaggcagcgcctaaggtcccgaggtgctgagactgtgctggcgtttgccacctgcccctggctaggccgtttctgggccccaagaaacgcctaccttggcacttagggaccagaagcctctggatgtctagcaacaggggtcacgggatcactgcgtggggtctctgtaagcagtcccctgaggcagtgcaaaaccggaaacctgctctgtgcggggccgaatcagttcatgggatttgggagtcaggagagacgtctttctcctcccagctcctacactccgggtaccaaggcccgaaatgccgttcccccagcccgggtgcggggtctgcagcaaggcctcctgatttgcaaacccctccgggctgcttattcctggtgccatgcgcagcccttgggacctagaagattgtgggggagggaaggtcgcacgtgcggttttggcaccgaccgtgcctcctagtccacctgtcccccacagctagctgccaactcggacggaagcgcggaacagcgcaatgcaaaccgcccaaagtgaaaggaatgtaattgcgctcccatgacagagccagacgcggatgcagtttaggaagggcgccgcctaccggcccctgggagccatgcgattcgaagggagggggacctagaggagacccccgctatccccccacccaccgtggggccttagctttgaactccggcccggacaaacttaaactgcttcgccaccccaacgcgccacagccctggacctaagacccagttagcattgggaatttggggagcagggcccacgactagaacaaagttaaccctaccggttcccgccacggcttcggcacatttcaaaaaaccaggcgcaggcaattgagaaatacgctgccggctgaaacagcctgcgggtgggggctgcagccgtgcgcgcccggcagttcccgtcccgcatcaggtgtacgcacttccactcctgcgggcccttccacgctccaactctggaccccgcgcagttttaatctgcggtttgggaaatgggggtgctaccgtgcaaccgcgccctgaaagaccgttttggtcttaagagcttttggcctgtggggtggacatctgtagaaaaaggaaaaacaaaacaaaagtaacctcccattgcgtcgaaccctcctattccgaaaagaactttaatgaggttggcttggcaaggcctgcggtgcttacctcggcttcgccctaccccgccagaagccctcataggttgtatccactggtctcccccaggtgcagggttattaggggaaaggggggcgcgccctcgggctggatctttgtttccctcgtcgcccggtcatcaaacaggagggaaatcgggcccgactgggaccttgctgcccgccttccccttaaactggctaaagcttcaggactgtccctagacccaccccgcgggtcttcctttgtgtccagggcatagccgatctccttttcgttttcacgagaactgcgacttgggcctcgggcactaggcgagcccaggttgtggcctaacagcagatcgcctcgggagcttggctgcagctctgcccgcacctccagcgctgggcggcctctcggggccagtggggatctctggcctcgtgtaggccacgggccccagccctgggtccccaaggccgctcgccgggcagcgctcgtttccggcaccgggacgagcccagcgcgctcagacaccactttcccggtgaaatctgcttttatttgctccgagcaaacctcgggctctcagcgctcccgcctgatggatgcaaatgtaaatgtgcacttatttaattggatcaggccccaagataaaagagataaacggctccccgcttgctaacttattttcctaggcatgcagcgccgcgtggaggggaagctaatgaaggagcagcgcggtgctgggcactcgagctctccgcagccgtggcacgctggccggccggccccgcg

SEQ ID NO: 275 - DNA, 947 bp, Homo sapiens
  source 1..947: mol_type = genomic DNA
cgcaggccaacgcgaagacacgatgatcccaaagaccttagtgtcttggcggggggagggggaagaggtgggggacttttccgcttagcttccctaagggttgctacgcgacagctctcgggctacgtcagggtaaagaggtttgtatggccacacctcgcaccgaccaactacccccgaaacaccccctcctcccaaactgcagggtgagcacagcgagagaaaaggtggcccaagcagaggcgaacaactgcggttcttaaagccttgtttgagtttgggcccgcgccgagccacagctagaggtagatcccgctcctgcgcgtccgctatcagccccaggattcccagagaggcctctcaggcccttcaagacccccccttccggcttcccggcctacacctctgaaccctcaggccccggacagcctctatccccgcctttcgcggccccggctcaccctctagcgtctcgcactgcaccaggttgaggtagtcggcctggctgagcaccccggccttcaggccgcgcaccagtccctccaagtagccattgtccacgttaaagtaaagctccgggaagaacgacatggctgctgcgggagcggcgggaccggagaaccaggaccggccggcacgaatcgcgactccccaggtcagctgacgctgcgtcctcagcgggctgtcaagtgcgtcaggtgacgcgcaagcgcctcacgtgaccataagggggcgtggctctgagcggtgcccggcttccgggtggctgcgcggaagcgcggtgcaggcgtgggctggccctgcggcgcctgcgcgctaaaaagaggaagtttgaggccggccgcttggtggcgcctgtggccgcagggatttgactgggccttctctagctttccccggtttgctgcacttgacgctgcagggcgggcggggtaaagaagggagcg

SEQ ID NO: 276 - DNA, 536 bp, Homo sapiens
  source 1..536: mol_type = genomic DNA
cgggggttgggggcacggaaggggaagcttcggtcgctaagtgggccggcctttggaagggtctgggaggggccagcgctgactaacagtcggtttccctaccctagacggctgcgacgaggagctggtgggtcccctgtatgcacgctccctgggcgcctcctcctactacagtctccttactgcgccgagattcgccaggctgcacggtgagctccgcggaacatcagctgccaactggcagcgcaccgcggaagggtgggggcctccggaggacttcggggagagggatagccggttaaagctcctgtcctttctataggcataagcgggtggtcaccacggattggggatccgaatccctggctccagatagacttaatgaagaagcaccggatccgggccgtggccacacagggctcctttaattcttgggactgggtcacacgttacatgctactctacggcgaccgagtggacagctggacaccgttctaccagcgagggcacaactcggtactctgggcgccaaggcg

SEQ ID NO: 277 - DNA, 874 bp, Homo sapiens
  source 1..874: mol_type = genomic DNA
cggcgctggcgcaggcggccaggagagagcagatgaaccgttcgttaggagcagcgaggtagtcgtgagcgctgagatccagagactaggacccactccctctctgagcagcaaattgggaagaagatgctcactcggtaagggcgagggagcccggcatggcgccccaccacgggctcggtctatctgcgcgccaagatcccgcttggggcgaggcgttgggtcagcgtttagagccactccctgcgctggtggctggacatagcctccctatcccacctcatcttcccccatccccgacagaggaggttgtgaatctacaggcccttgacgttgaggcgtcggagggcgcacctttgtaattgcggcctcccttcgccccttaagtgccgcttctgggcgcctaggctggatatgaaagccccgttcctaatcctctgctctggtcccctcctctggactgctgggactctaagctaggccctccccaggttccatcactgcggcgccaacccgcggctgggctgtccgcaagagggagttgaaggcgcgcggaatcccgaggtgcagctgaccctcctctcaacgccgactctgccgctcccgcccggccacctccctgtcgggcagacttcctgttctcctgctcacagcagggaggcagtcgccgagccggtcagcagcgtgcacggagatcttcactctgcgcccagccccgggacacaggtgcagatctccagcggagcactgcggagtgcgcgccgtcgagcactagggaatcctagacggaggacttggtccattccacgcagtcccaggcaggtccgcagcggagggacgcagcggtctccaactcctggtcacgacttcggcg

SEQ ID NO: 278 - DNA, 3724 bp, Homo sapiens
  source 1..3724: mol_type = genomic DNA
cgactcggctgacgtttttgacccggccaggaggcaaagaccaaaacgtcagagcagtagccctgttactgaggagcgtcggcagggtcgcgggtagagggggctggagaatgacttgtcagagctcaaggtcgatgtggcgcggggcggcctcgagagcgccgggctcctgcgtggccacggccgccgctgccaaccttcgcggggacttagctttgctttccattgactccctttgcaaaagcgcagcagaatcctgaccagccgcaccagccccggcgaacccgagcatgttaatctatttatatggattattacggaggaacagcgggcgttgagtcaccaaaacatttgcttcaaaagactatttctaagcacttttgcaggcaggcaggctcgctccaggcgcgtaaactcggctacgcattaagaagcggctgcttttcgaatactgcaaactccagctaagtccccggtgccgcggagagagcagtgaaaagaaatgtcggaggtgggggtagatcctagtctagacacacacacttgcgcgcacacacacacacacacacacaagattcgcgcggagaaggcactaaaattctggcattccgagagtacgacaaacttacacacttggaagtcccgggtcccccgccttccccgcagcaccccccgcccccccaccctaccgtccgccctttggctgcgatcccctcccctctcctcccctcccgcctcgtcacccagcccagtgccacaatcctcctccctccccaaaatcgggtccaatcagctgcctgccaaccctgggactgctgtgctgtgattggcgggtggctctaaggtgaggcggagtatttattaaagagaccctgggctgggagttggagagccgaaagcggagctcgaaactgactggaaacttcagtggcgcggagactcgccagtttcaaccccggaaacttttctttgcaggaggagaagagaaggggtgcaagcgcccccacttttgctctttttcctcccctcctcctcctctccaattcgcctccccccacttggagcgggcagctgtgaactggccaccccgcgccttcctaagtgctcgccgcggtagccggccgacgcgccagcttccccgggagccgcttgctccgcatccgggcagccgaggggagaggagcccgcgcctcgagtccccgagccgccgcggcttctcgcctttcccggccaccagccccctgccccgggcccgcgtatgaatctcctggaccccttcatgaagatgaccgacgagcaggagaagggcctgtccggcgcccccagccccaccatgtccgaggactccgcgggctcgccctgcccgtcgggctccggctcggacaccgagaacacgcggccccaggagaacacgttccccaagggcgagcccgatctgaagaaggagagcgaggaggacaagttccccgtgtgcatccgcgaggcggtcagccaggtgctcaaaggctacgactggacgctggtgcccatgccggtgcgcgtcaacggctccagcaagaacaagccgcacgtcaagcggcccatgaacgccttcatggtgtgggcgcaggcggcgcgcaggaagctcgcggaccagtacccgcacttgcacaacgccgagctcagcaagacgctgggcaagctctggaggtaggacccggcgggggcggcgcggcagggtgggcatcgcggcggctgggggcgctggtcagggctgatttgccccgccccgcctcccatcgcccgggagttgccgttccgggagccggcgggatggggttgggagtgggaatggggtgtaactgtggctcagagtttgacaaagttcttgggctgctcgcggggacgcggaggaggggggtggtaagtggaagaggtgagggaggtagctggaggatggacgaagactggtgggagacggaaggagggggctgccagcctgctctccagtcgcctggaagctcaatcggggcggggaagtgaaacttgcctccctcctacccggcctcttaaaactgcactctctcgtgcagccccactgtccacggagatggggcaagggagaaaccgaggttggaggagacccttggcaggaactgggaggcgggaggagggaggctactggaaataggtgggagtgtatggtggggggtgagaattggggaccttcttgcagcttaagtaatttgggggaaagttttcaaagggggttggggttgggggcggtaagtcgagcagcaaaggcgtttagggggcagcaccgggagtcgttttcatctccagcgtttccaaaatagaaatagaaggggaggggagggagggggcggggagtgaccgctcaggtcagactgcaataacttatttatttatttatttttaagaaaagttatgagctgtggttgcaggcaggagggaagatggagttgtgtgcagaggaagccgagtggtctgggtcgccgcctcctccccgccgacctgacagtttggcggatttcactgacccctctccctctttttctctgtgccccccgccccgccccgagcagacttctgaacgagagcgagaagcggcccttcgtggaggaggcggagcggctgcgcgtgcagcacaagaaggaccacccggattacaagtaccagccgcggcggaggaagtcggtgaagaacgggcaggcggaggcagaggaggccacggagcagacgcacatctcccccaacgccatcttcaaggcgctgcaggccgactcgccacactcctcctccggcatgagcgaggtgcactcccccggcgagcactcgggtgagtcgcccctcgaccccaccggacaagctatctccgtcccgcctggcacaccccctgccctccgcctgggagattcttcgtggggactttatgcttcccgggagggacacactgccctttgcgcccgtcccgctcccctctctacccagagcctaagaggcatccaaacaacacacacacaaacacacacaccccaactcaatcccagcatccgaagagattaacttttttattgggaggtaaaatgcccttaacagccttacaagacctctcccttcttctctgctcccccaccccaaaagcacacacagggctcttacacaagtagcaattaggtcttccggaccctccgggccccagaccctcccctgataaaagggggctgtccagtgtgtaccggcgggttaatcattgggcgacttatctccggtgcagcgcgcctcttgcgcgggtgcgggcccttattacactttagcagcgagggagggtccccggagggtgcctaagactagggcgtctgcacagcccttgttgattttctcgtgcttgttcttttattgtccacagggcaatcccagggcccaccgaccccacccaccacccccaaaaccgacgtgcagccgggcaaggctgacctgaagcgagaggggcgccccttgccagaggggggcagacagccccctatcgacttccgcgacgtggacatcggcgagctgagcagcgacgtcatctccaacatcgagaccttcgatgtcaacgagtttgaccagtacctgccgcccaacggccacccgggggtgccggccacgcacggccaggtcacctacacgggcagctacggcatcagcagcaccgcggccaccccggcgagcgcgggccacg

SEQ ID NO: 279 - DNA, 3126 bp, Homo sapiens
  source 1..3126: mol_type = genomic DNA
cgcctccgccccgcgtttcctccccccgatctcccaggggtcctcttttgagggcaccccagcccctcgacggtcccgggatatcctgcacccctgccaggctgcagggaagcgccgaggcgcgcggagtgcgaggtccgtgctccctgcggcgcggcgcacacgcagggtccggggtcgacccctccaccgctccggcgtctgccggggaagtcagtccgcctggtcagcccagaacccccgactgcgggggctggagctcggaagcaggtacaagcgccactctccgcctgcgccgtggaatgcgcgccgggaccactccgcagcccttcccccagcgccgccggccgctgctggggacaacctcgccctcctgtctcttgctcctcctcctgaccccagcgcacccccatccccgccccagatgaggcaaggctccctccgccttcagcccggcagagtcgcactaggagttgcagcggccgcagccccgggagcttcccgctcgcggagacccagacggctgcaggagcccgggcagcctcggggtcagcggcaccatgaacgtctcgggctgcccaggggccgggaacgcgagccaggcgggcggcgggggaggctggcaccccgaggcggtcatcgtgcccctgctcttcgcgctcatcttcctcgtgggcaccgtgggcaacacgctggtgctggcggtgctgctgcgcggcggccaggcggtcagcactaccaacctgttcatccttaacctgggcgtggccgacctgtgtttcatcctgtgctgcgtgcccttccaggccaccatctacaccctggacggctgggtgttcggctcgctgctgtgcaaggcggtgcacttcctcatcttcctcaccatgcacgccagcagcttcacgctggccgccgtctccctggacaggtgagccagcgccttggcctccctgggagatgggcatccacgcgggggatggagcgggaggcgggactggggaccaagaagggacgcgcagagtgggacaggacactaagaaggcagtggaagacaagcgggcgcggaggaggaaaaagaggaataagaatgggggaccgtggtgtccctcggttagatgcgtcctggggcctggaagcctggagaatgtggctctccagcgccgcccgtgcctgacaacgcgcagcgtttcccagtacgacgcgtttgtgcgcgttcatctcgcttgagcttaatgccctccgtgagggtgggataggacaaagtgcccaatatacagaagagttgagttcctaagtaactcgctcagagtcgccagccagggatcgggtgcgtgaagtgaccgtctgtctcctgcagccaacttcaggcgcctccactgcgctcgcctccaagccacggtttggttggttggtgcagctggctcaggtccaggctgtggatcttgggtcctttgcaaggatccactccggagtcccagcgagcgtgcctaaaggtccctagctcagtcccagcccactctgcctctcgcctccaaacaaaacaaaacaaaataaaatccaaaacaagtcggggccgggagaggagcgtgccctggggttcttcctccccagccagaggagagcgagagacgcacattcgggagagcgcgggactcaggtggagcttgaaaggacactgggatggttcctggggaggaaatccgggtatttcccctctccatcctctggaaaaacagagaggcgaggccagactgcccccacacctcctgtagccactgagcgcgaagtgcgttggttccgagcgcgctggtgggatccacaaagctcgcattctctcaggaatcccctgagaaattaactgtcccttgcccaacatgtcttctccaggctgtctgctagagcctcaggcgcctccgccctccctcccgcggcaccgtcaccagtgggtagtcacagcctcccggagcccatagccggttctccaacctttagtcttcagtggctttggggtgccctctcagtggagactgtggttgcagtccccgggggcagcgggagaatggcttgaaggcacacctttcctgctgccgggcccgccccatttccaggctccgctgagtgtctgggacacgctgggaggcccccacctccgccctcacgccgagcctcacccccacctcctctgtgtgcggtgtaaccatgcgctaaggaccttcctcgagagcagccttgggaccgaggtgcaggggtcgcggccctccagcatgaatgtgcccgctcagccgacgtctcccttcccggtctgaccgcaggtatctggccatccgctacccgctgcactcccgcgagctgcgcacgcctcgaaacgcgctggcagccatcgggctcatctgggggctgtcgctgctcttctccgggccctacctgagctactaccgccagtcgcagctggccaacctgaccgtgtgccatcccgcgtggagcgcccctcgccgccgcgccatggacatctgcaccttcgtcttcagctacctgcttcctgtgctggttctcggcctgacctacgcgcgcaccttgcgctacctctggcgcgccgtcgacccggtggccgcgggctcgggtgcccggcgcgccaagcgcaaggtgacacgcatgatcctcatcgtggccgcgctcttctgcctctgctggatgccccaccacgcgctcatcctctgcgtgtggttcggccagttcccgctcacgcgcgccacttatgcgcttcgcatcctctcgcacctggtctcctacgccaactcctgcgtcaaccccatcgtttacgcgctggtctccaagcacttccgcaaaggcttccgcacgatctgcgcgggcctgctgggccgtgccccaggccgagcctcgggccgtgtgtgcgctgccgcgcggggcacccacagtggcagcgtgttggagcgcgagtccagcgacctgttgcacatgagcgaggcggcgggggcccttcgtccctgccccggcgcttcccagccatgcatcctcgagccctgtcctggcccgtcctggcagggcccaaaggcaggcgacagcatcctgacggttgatgtggcctgaaagcacttagcgggcgcg

SEQ ID NO: 280 - DNA, 526 bp, Homo sapiens
  source 1..526: mol_type = genomic DNA
cgagagcctctacgacgcactcaaccagtactacgtccacctcggcggccagaagtacgtggacctcggtctggggacccaccgcgtcaaatgtcgggttcaccccaacttccgcctgattgtcattgaagagaaagacgtcgtgtacaaacactttcccatccccctcattaaccggctggagaagcactatctggatatcaacacggtgctggagaaatggcagaagagcatcgtggaggagctctgtgcgtgggtggagaagttcatcaatgtcaaagcacatcatttccagaagaggcacaaatacagcccctctgacgtcttcatcggctaccactcggacgcctgcgcgtctgtggtgctgcaggtcatagagaggcagggtccccgggccttgacggaggaacttcaccagaaggtgtctgaggaggccaaatcgatcctgctgaactgcgctacgcccgatgccgtggtccggctgagcgcctactcgctgggcgggttcgcagcggagtggctgtcg

SEQ ID NO: 281 - DNA, 242 bp, Homo sapiens
  source 1..242: mol_type = genomic DNA
cgtcacgtgcagagcatcctcccgactggcgaggtggcttccacgtgccccgtcaatcagggctggacagtccgtcacgtgcagagcatcctcccgactggcgaggtggcttccacgtgccccgtcaatcagggctggacagtccatcacgtgcagagcatcctcccgactggcgaggtggcttccacggagcgattctcgagacccgaggggcgtggacacagcttctacgcaggcagtcg

SEQ ID NO: 282 - DNA, 724 bp, Homo sapiens
  source 1..724: mol_type = genomic DNA
cgcgcgtcgggcgctgggaaggtgactgtgcgttgggcgcgcttggtggccccagggctgggcctgctcccgtctgacagtagtgccgcgctgcatggcacgggtgttgcgttcgtgggtgagagcgctgaggtctgcacgtcgggtctcagtggtcatggggatggaagccaccccagtggctgtcggggtgaatggaggcccctggagatggcagcgggttaagcacgagcacggaattctccctcccaaaacggaactcagacaacagtgacggagcctaagagcaaagccaaagccacgggcaggggtgagcgggacccgaggcaggagatgacggagcctaagagcaaagcgaagccacgggcaggggtgagcgggacccgaggcaggaggtgacagagcctaagagcaaagcgaagccacgggcaggggtgagcgggacccgaggcaggagatgacggagcctgagagcaaagcgaagccacgggcaggggtgagcgggacccgagccaggagtggcgccgggcagggaggcaggcagccccaggcgctgtgtgggcactgggtgagaagcagggatcccggcgccctggctctgtgctgcttcctgagacgagagtggcagagggcaagaagaaaaccaaacagcgcggggaacagaagtcaggaaagatgaacgtctggaaggaaatgcccctgggccgggcggtgcccccgggcg

SEQ ID NO: 283 - DNA, 4996 bp, Homo sapiens
  source 1..4996: mol_type = genomic DNA
cggagagacctcggagcagagaaggcgccgccgaccctcgcggctgcctggcccgcggctcctacaaaggcgggctagccgcccgccctctcccttgccttcctccccttcttttctgactttccctctttcccttaatcgcctgcttcttcctccgggtggacttacggccaccttgctcctccgcgcttcacctcatcgccccctctttctttcttctgcctctctctctgcgcccccttctctccgtgtcacgctccctcctggttctgcgcgtctacaaacttttgagcagaacacgagcctcggcaaacgagtcccgcagctcctcctgctgctcccgctggttcctgcggcttctgctcagacaccaacgccagacggcgatgcctctcgggtggtgactccagcgcaggaacttgaagaagcgctttgcccgccgtcctacctggcagctctcctggcagcgggaggagttgaagggtaagggagggaaaatcttaccaaagcgaccggctcactcgactgctgattctttcgcttggcatcgcgtcaggggagttagctttccttcagccgggtctggctagttattgggcgccgggtagatgcatatatatatatttttttctaactatagcaagcaagaagtggcagggcgcgcaccggctgtcgccaagtgctgttcaactcagggagccggggcttcgctccgtccctcccccggcttccagagctttttggggttggagggtgggaggccaggggcgttctcacagctgtgtgtcctctttcccatcctgcgcagaatgaccatgtgtagcggagcgaggctggccctgctggtctatgggataatcatgcacagcagcgtctacagctcacctgccgccgccggactccggttccccgggatcaggtaggtgctggctgcctggcccaagcaggagctggggcttcccaggcacagacgcttcctcacggtctccttcctgcagtcctttgggtccagactactagcatcgccctctgcgcccccggtgcgcctccgccagcctcggctggacagcgggtccccattctagccgagggtctggcaggctccgcgactgctcggacgcctcccccagccctaggcagctcagggtcccgggtagagccagtgagcttctggccgctggagaaccccccctcccccaacccggcccacaggatgggggcagggcacggcccctagcttggtttcttttacctattcttgggacgagttaggagaacttcagctctggagcctggccgggggttgagcgtgaagctccctcggactttgctttgttactgcttgttctggactatccgggtggggtctctctctctcctccaccctttcttttcatttcattccaattctttcccctgaagagctttctttcaagtgatccgtgttccaactgcattttgaatcccaggctgtcttggggggcgtgcggtggggagggtgttggcccggtgtgattgaggaaaagcgacttaagagagggaagaacaaggacgagactgcgaaggagggggaaaaacaggcgcaaaggaggaggaagggaaagccagcaggcaggcaggaccgggagagcagccctgcctggcccgggatggaggaaccttggcttttttcttaaccccgggtttctaaccggcaggcgcggcccaggttcccggaggcagccccagagtcgcggaccgatgtgccaggctgtggatgagccctgggtaggggagggttcgtaccagcggcgcctggggcagcgaggagcgcgcgttctgcctgcgaagctgccttctccgagccccgcccaggaacattagctctggggggccgctgatcattgatttggacggagagatgggttctgggttctgtattaggattccagcatctgggctcgaggcagggcaatatccagaaagaccccagggttcggggtacccgggccagggctgaggcgcatcgccgagcaaaggctgggtgcgaggcgtgcggaatgatgcgcttgccttgcccgggcctctccaaggatggagaaaaggcgagtgaagcagcgaagtacgactccaaccccgcccagagagtgctactagcgctggctgcacgccaagtctctccaggggtccaaagcgagagggatttgttttaacccatctctacccgtcctgtgtcaagaacggaggctgtagagggcgactgcgaagtcgccaggcactcgctggatctcggtccccctcctcgtgctctggggttgagatggggcaccgccatcgataacagatcagcgcgaactattcgtttagtggccttaaaacaccctggtttcaccctcagctattttcaagttcccgtgtgcctggcactttctccgtgcgagaagcaccggagggtgcggacgcgccacagtctgagccgccgccgaactggctaagtttaggggcatttattattcatgttcctgccagatcctcgcctgcccaaaatagaaaccgaggttctccgtgacctacatctgctcggagaagggctcccctgggctcggaggctggggtgggggtggctgaggagttggcccccgcacgccccacgcatcctctcctttgctttctgggcctccccattcgggtcttcgcgtgggtcagcgcccggtctcccagggcctttctcgtccccgcccgttgctgctttggggaggctcgggagccaggcggggaggggggcggtccttttccgtagacaggtgtgcgcgatcggcggagacgcctcggtttcccagcgcttgttgaggccgtggcccgcaggacgaccctttacccgcgaagggggggtgggcgggccgcccggcggggtaggagtggttgggtgtcgttgcctcctccttacctctgctcccacccccagtcctgggagaagagacaattctcagcggaggacttttatcacctgtgaaaatccgcgcgagccccttactttggatcctcgccgagctggggaggaacttgcactgaccacaccttctgtccccggccaccccgcaggccagaggaagaggcgtacggcgaggacggaaacccgctgccagacttcgatggctcggagccgccgggcgcagggagccccgcctccgcgccgcgcgccgccgccgcctggtaccgcccggccgggagaaggtgagattcgcgcggcctcgcgcacacccgcggctgggagctcgggactgcggtgacgggaggggcagtgtggtgacccacccaggatttttttttttttcccgtgaaagtcctcaagcctgtcctctccctggcccgatcctattgcagcgacagaaaatcagcagcgggcgggtctgtgtggacctgagggccgcgtggggaccgaggggggctgtggcccaaagagtggcagtgagtggcgtcaaggaacccacactccgcatctgccactcctagagccgggactagctcccgatcctagcagttgctctcgagatcatcccgggagttattggcgagttctgggcctctggaggtttccctgtcagcctccccggccgccgagggggcgcgcgcccaacaagggggtctctagcggccacctggggacagaaacagtgaccctgggcgcgcactttgcctccccgttagagatgtcgcccacgggatccttaacgaggcctaccgcaaagtgctggaccagctgtccgccgggaagcacctgcagtcgctcgtggcccggggcgtggggtaagagtttgtggaaggattaacctgcgcgcgccggggtgggtgcctgtgcggggcgcgcggggcgggcggcggtgggtgcccgtgggggccagggtgagtctgcgcccctgggtctggggtgggcatccgccacgggtcgcagttggagattttgaagtggcactttaaatttgcccagagagctctggaagaggcaaaaagggaacgcgagccagggagtttgatccgttttgaatgaaaagaaagagaaaccaaaccaaacctctcagtcatccaaaaccttcaggcttccagggaggttttgctataattttctctaagcatgactgtttctgggggaggggaaaggggtggttgtatttactgaaaattcaaatcgaaataataaatggccaaatgtggacacttatggacccaaacagttttgctcacgccagagaaactgagagcacagggcttgtgtgaagcctatctcggcagaaggcaacattctaataaagcccgtgggaaaacagattacattttcgccatgaataagtcatgcagtgaaaaatattgcctacagcctgtcgacttatattattatcacgtttttcaactcggcgtgaggagggagaggagtgttcatatttgactaggaattgcaggatcgatgcaaactccagggcagcagccagactggcatatgtagggctctccggttactttctctgtatgtcgcgggtgagaggaacagcgaggacaatttagcgcaaacacacgaagggtcggatctcaagggggcagcgctgggagaaaggttaggcttgaagcgcgcgtcgcctgcccggatcttatcccgggccccctccgcagggtttggtgccaggagatcctgcgtggggaggggggcatcgaggggctgccgtctcggccctccccacggctgcttccaggcagaggcgggcgacgcggtgggcagtgcgagccccgggccctccccgaaggctcccgcgtggggtggggcccgcctgctccccgcggcgattgaacctgtgtctcccgccccgccaccctcttcccgacccctttgcttgcagtgggagcctcggcggcggcgcgggggacgacgcggagccgctctccaagcgccactcggacgggatcttcacggacagctacagccgctaccggaaacaaatggctgtcaagaaatacttggcggccg

SEQ ID NO: 284 - DNA, 4239 bp, Homo sapiens
  source 1..4239: mol_type = genomic DNA
cgcagaaattactcgtgcgcaccatttccgctgtgggggcattcgtacaagtttccgctgcacacacagcctcccgggccctctcctccaaggctctgccggatcttccaacgaaatcccagagcagcctgcgctggggagcccgcaagtctctccagatctctgcaccccgcaccgcccggaatctgggacggcgcccacgcagggctgggccaagggcagagctcgcaccctgccttcacgcccggttcacttgcgtccacgaaagcagcgtgccggcctcctccatcttcccactcgcgcaacgcacggcgacccgcgcgacacttctgcaatctgaaggcttgcttcttacaaataaagggccagagtctcacacttgccttcgttggagggacttagaagatcctccccacgtccacaccttgtaggaaatgcaaaacagatcgatgaaattaaacagttgcatttggaagccccagaaagacctaaagacatcgtgccggtttgttggagagagggttgcgggacagggggagcgggccttacgcaacagaaaaggtgggcacagcgcgctcaaaatgacccagtgaggagttggtgccgccgggccagaggctgcgagtccagctggctctggacttgctccgcaggcgtcagacgccgtgggaacctgtgtctgcttcttctctccaaagtgtatcggttaaaaaaaaataaaagtagtagtagtagtagtggtaaggaaaaaaataaaaataaaaaggagacacaattaaccaggtcataaaagctagggcaccttcgaccagggctctggccctccagcgatcgttttgcgttgtttctcttctcaaaagtagtctcagacccctgcctttccgctgcagctctgcgacttccccaaactccttaatcctgtaaattctgcaagaaactcccatcctgcaagctgcttttccccctcccccctgcgttccttttttctctccccacccgcgccgcctctctatgcccctctcttctcagaaaaattcctgccccccgcgcgccccaaagcccgggctgcaaacttttccccgccgggcgcctctgcgccagatgccggagcgtctccacaaagcctgagcatctgcacaagttcgcagcctaactgcgggataaagacgtttcccccgtagcttaactagaaaagcgccatcgatgggtgtgttaaacgggataactagagatttcaaacaccttttatttgcctgtcttgaaaaaaaaatctaaatgaatacgcccgctaccaaaaggcaaaataaaaccaaccttaagggtttttgttgttttttttttttttcaaaagtggcgatagggactgtttggacctgactccaacctgcgccctcccttcctctatgaccctcctgcgcttttcctggaacccaaagctctgacttcgtcaaacttacacaattaaaggcaggcggaagaacgcgggctgggaagcaagcgggaagattctagaatggaagggagcccgccgagcgccgcgagccgcgccaggccgggtccgatggagcaggcggggattcctcccccaggcggacccccgccaccagccctgccgggagctcgcggcctgcggagcgcccgggctggccgctcaccgcccgcttcccccagcgaacgactcggggaagctccaggaggccatctgtgctgacggttcacaccagacaggaccacttgcaaggacaaaaataagaaatttaggaaacgaaaaaagacgtactggggcgaggggcgcgggcgcggcgacgacggggccgggggcacatcctggcggccgctcggggagagaggacacgcgcgggaaggagcgcggcgggtgcacggccgcgggtgggagtacgcgcctgtgcgcgcggggcgagggcgagggcgcgtgcgtgtgaccgcggggagggggcgggcgcgtgtgcggggagcgcgccgcgccaggggccgagtgtgtggggccgatccagaagtgcgcagccccctcacctggcccccgtgtcatccccgaaatcccgggaaagggtgggccgcgcgcgggagtttggtggagttggaactttcggtcgcgctcgctgcccactccgctggcgcccggtggcccgtggtgaagggggactagggtggggaacaccggggccctgcggtcccctccctttcctgtatttaagaagccgccggcggcgcagaggcccaggcgggctggcgcgggggcgaggcggcccggtggcagcagcgggcggggcgggcgctccggagtcggtggggcccgcgggttggggggcggggagaggggggagtggaagggagggggaacgcaggggagggagaggaggggaggagccgcgcggcccgcgccgcttccgaaccggaaagttggtcttgccgaagtcctgccaccccggcgtgcgcactccgctccgctccggccgcgagcctccgagcccggccggccgccgggggaagcccgcggaggggacgcggggccgggcgagaaggtccggagagcggggggcacctgagcccgggcgggcccgccgcgctgagcggcgctgagagccgcggcggagcagcgaaggcggccggccgaccccgcgcgcccggaacaggaggcgcggcgcccgagcggcccgggcgagacaaaggcgccgggtcggagccctgcccgcggccgctcgctccgggaggggccgcccggcggcggcggcggggggggcgcgggcggcggcgcagacactctataaaggggcgagcccggcgcgccggcggagacggcgccgcgcggacgccgccaaagtttgctgcctgcgccctgcggagggacggccaccgcggcccgcgccgcacccgggccccgccacagccgcacccggggcggccgaggagcgcggcgccggagcccgcgatgtgaggcggcgccgggcagcgcgcgccccggtcccgaggcgccgcggccccctcctcgtcggcgcggccgctaattgcgagcgcggcctcatttgcataggccgccggagtccgctggagcccggccaatcggcgcggccctccgctaatggccatgcattattcaccagcctaattgctcagccccatgcgcggcccgcgcagccgccgccgccccgcgccccgcgccgcgcgcccgccaggccgccccgcgccgtccccgccggccgccccgctgatgccgctgccccgcgcggggcccgagcgccgctagcagcatgtctcggcgcaagcaggccaagccccagcacctcaagtcggacgaggagctgctgccgcctgacggggctcccgagcacggtgagggccggggctgcggggtggccggggggtctggggctgcccgtccgggctggggaagcgcgtgcggcgggagcggatgcgcgcgtccgggagcgggagaaagttccctgcttcctgcgggcaagcgtccgccccgcgccaggccggccgcggggccccgggtacttcgccggagcgcgcgcggccgccgagagagttgtgggcgaagtaaacttggctcctctcctcggagtcggggagctgcccgcgaagggcgccgaggccgcggccggctcgaggacggctcggaggccggggcgggagggagtccacggtgcctccgccgccgcgccgccccccagggtctctgcgccaggacgctgaggccggcggcggcggggaaggcgaccgcagcccacctaccgctggacgcgggttggggaccccgccgcccggccagctttgttcgggggcccgcggcccctcccgggcccccgcaccgcctcgggtgacccgcggtgtcccagcgcgttgacgcagcctgtgatccctcgcgaggcgaggagaaggtcgggggcttggctctgcctaatggccgcccggggaattaagctgggggtgagcgcagcggcggcggcctgggcctggcccctgctcgcggcgtgtttccggggcgttcgttgcagcgtctgcgcgggccttttctctcccgtctttttggatccgccgaggccgggcgctggagacctcggctttgcagtcatttcgctggtaggagcgtcctcttcgaaacatccaagagcaaagggcaggcgccgcgaaagttaagagactggcaaagggctggacttcccagagtggcgccttagccccgcaaagtttggggcgcccccacccccttcgtcg

SEQ ID NO: 285 - DNA, 621 bp, Homo sapiens
  source 1..621: mol_type = genomic DNA
cgggtgcaaacgcatcctctgtcccacgtggagtggtgtgtccacacgcatgtcctctgtcccatgcccacgtggaggggcgtgtacacacgcatgtcctctgtcccgtgtggaggggcgtgtacacgcacacgtgtcctctgtcccgtgtggaggggcgtgtacatgctcacatgtgtcctctgtcccgtgtggaggggcgtgtacatgcaaacgtgtatcctctgtcccatgtggagggatgggcacatgtgtgtcttcaggacgcctgtctcaggcacccgtgtgtcttcaggacgcctgtcgggcacacgcgtgtcttcagaacgcttgtctggggcacacgtgtgttctcaggatgcctgtctcgggcacacaggttgcgggggcggccggggctcctcaggccttttctcccgactgttgctactattctttttagggcatctttcacttccagctgggccggggtgagcccatgctgcacacgcgcctgcgtccacactgcaaggacttcctggagaagatcgccaagctgtacgagctgcacgtcttcaccttcggcagccggctgtacgcacacaccatcgcaggtcagtcaagccgcagccgaagaggccg

SEQ ID NO: 286 - DNA, 434 bp, Homo sapiens
  source 1..434: mol_type = genomic DNA
cgggaccgtttcacagatgagaagaccgaggtgccagggtggcgatcgcacgaagggcccgtcacgggagaatgaggagggtctggcgtcagcaagctcccccggaatctcgtgatctggaaatccacagagaagcaccttcaccacgccagcttcccgggccgcgcttcattgtcatttctggagagtgttattaatatccgccccgtgggtgcggcgctgtgtatttccagagaggggactcggggcccatcagggccgggggagtggagccggcagatgttcggccctgcccagaatcacttccccaatttccccacgcccggcggggctctcagcatctgcatccgccgaggcctggctgtgctgacgggaattgaaaggacctcgtggcttaggcaggcgggggagccccccggctggtcaggtgcacg

SEQ ID NO: 287 - DNA, 1012 bp, Homo sapiens
  source 1..1012: mol_type = genomic DNA
cgccgcgactatggggtagcgttcaagggcaggccgcacctcactcagcacatgagcatgtacgacgggagaaaaatgcatgaatgtcatcagtgccaaaaagccttcaccacgagcgcgtccctcacacggcacaggagaatccacaccggggagaagccttacgagtgcagcgactgcgggaaagccttcaacgacccttcagcccttaggagccacgcaagaactcacctcaaagagaagccctttgactgcagtcagtgtggaaatgcattccggaccctctcggccctgaaaatccacatgcgagttcacactggcgagaggccttacaagtgtgatcagtgcgggaaggcttacggccggagctgccacctcatcgcacacaagagaacgcacaccggagagaggccctacgagtgtcacgactgtgggaaagctttccagcacccctcccacctcaaagagcacgtgaggaatcacacgggggagaagccctacgcgtgcacgcagtgcggcaaagccttccgctggaagtccaactttaatttgcacaagaagaaccacatggtggagaagacctacgaatgtaaagaatgcgggaaatcctttggcgatctcgtgtcccggaggaaacacatgaggattcacatcgtcaagaaacccgtggaatgtcggcagtgcgggaagaccttccgaaaccagtccatccttaagactcacatgaactctcacactggagagaaaccatacgggtgcgatctctgcgggaaagctttcagcgcgagttcaaacctcaccgcacacaggaagatacacacgcaagagagacgctacgaatgcgccgcctgcgggaaagtcttcggtgactatttatcccggcggaggcacatgagcgttcaccttgtaaagaaacgagttgagtgtaggcagtgtggcaaggccttcaggaaccagtcaacgctgaagacgcacatgcgaagccacacgggggagaaaccgtacgaatgcg

SEQ ID NO: 288 - DNA, 300 bp, Homo sapiens
  source 1..300: mol_type = genomic DNA
cggaaagcgcgcttttcggcagagctggttagtttttaatgcgggctttttttttctcacccgccgttccctccgctccggtctgattcgcaaatccccaaactggcacaaaccgggaaacttgcggccggccgttcttctccccgagttggagacagccctctcctcttccctcccccgcctctgggccctgacccactgcctggtttgcgctagagccgtttaaaaaagaaagaaaggaagaaaggagccgggctctggtaccccgatgccagccgagggcgcgtttcccaggcggcg

SEQ ID NO: 289 - DNA, 310 bp, Homo sapiens
  source 1..310: mol_type = genomic DNA
cgcgaccacccgctgcgcatgcgcttcacttgggcgtcacccggggaactgcgcctgcgcagtctctccccatccgaggtccgctccgcgagtgcgagcgcgcgccaggcccacccgggcgctgctcttcatgtccccgcggtcgaagacggccacatacgtccccaagaagacgtcaccgaggatccagaagggccctgcaggcggagggacatccagggcctggaaaccggacaagcagaggcggacgccatttcgagtagtctgcaaggcaacaagacgacaactgggtgtcgcggccacaaggacg

SEQ ID NO: 290 - DNA, 527 bp, Homo sapiens
  source 1..527: mol_type = genomic DNA
cgattcgcttccccaccacgacgccctagcgctactgtgcaacgaagacctcccaagcactggttccaatgcggagaccatgggctcccagactctgggaactccaacacgactgcgaaacgaactccgagcgaggactccccgagagctccccgcaacacggacctcacgcgctagcgaacaacagaaaaaaaaaagcgcgctctccctgcccctgaaacattcccagaagcccacgcagaccagaccgatgacctgtctccactgctggaggcgagtcagggacccgaagtctctaaacactcgcctctacccgccgccccgcgaaccccacacactgcagacgcgacactcgcaagtttcggggatggcggccggcgagggccatactgcgtctttccggagacacggaatacggcaccagccgtccctttatgatgcaatatgtctgcgcccaggggacgcttgctgggagcagccattttcaaccctactgccgtagagcaggcggagtccctcttttcgcg

SEQ ID NO: 291 - DNA, 2075 bp, Homo sapiens
  source 1..2075: mol_type = genomic DNA
cgaggtgggggcgaggaaccacccggactgggtctccatgggcggggtcgtggcttagggcagggacaggtgtagggcgaggggtgagttcggggcgtggacgtgcgtgggttcacaggtgtgaacggtagccgcacgtgggctgggactgagctgaaaaatcggccaggggcgaggcccgggtaggaagtgggtgcggcgtggggaggcgtggcctgacggtgtgattggcaggcggagctgatccgagaggacatccagggggctctgcacaattaccgctcgggccgcggggagcgcagggcggcggcgctcaggtgagagggaagaagttggcagggtctctgggaagccggtttcccctccttgtgcctcagtctacaacaccagcctggaacagaacaagagttttgcatggagtcaagcacaccctagtcgagtcttgtctgtacctcccagacgagctgaccccttctccagaactctgcttcttttctctgttccctgtccaggccctcagtttcactctagagaggtgctatccctccgtatatcggatttctccctacctcgttgaacttgttcactccctttgagccttttgagcctgtgtgtctcgttctgcgccctggatttccccctccctggacccctcagtggacccagtcttggtgtccccgtcgccctccgcagggccacgcaggaggagttgcagcgcgaccgctcgcccgccgctgagaccccgcccctgcagcgccgcccgtcagtccgcgcagtgatcagcaccgtagagcggggcgcgggccgcggacgaccccaggcgaagcccattcccgaggcagaggaggcgcagaggcctgagccggtggggacctcgagcaacgctgactcggcctccccggacctgggtccccggggtcctgacctggcggttctgcaggcggagcgggaagtggtgagccgctaaggaaggggtctgggggcagggccaggcgactggaggcggggctagggcgtggaagggcggggccggctgcgggacgggcgttctctggtcagacttctgcgttatggaagaggggctgggtcgggggcggggcttggttgtggggcgtggccaggtgtttggggcgtggcctgatctggggaagtgtataggtgctcaggttcagggcttcgacggggatggttttggaactcgggagccctgagcgtccccctcctctgtcccctaggacatcctgaaccacgtgttcgacgacgtagagagctttgtatcgaggctgcagaagtcggcggaggcggccagggtgctggagcaccgggaacgcggccgcaggagccggcgccgggcggctgggggtaaggggcaccctggcgtgggatctgaaccccctcccgatctcttccaaatgtccccgctctccccaggctctcccctcccgccacttgccagggctgacctcaccgccatcttaaccgggtgtccacctctctctgcctgcctggtgctggccccgcgtccccatcgccgcgcccgtctgctcccctcagagggcttgctgacgctgcgggccaagccgccctcggaggccgagtacaccgacgtgctgcagaagatcaagtacgccttcagcctgctggtgaggacgcgcccgcccctgggccggggcgcgggcacgacgaacctgtcccgtccccgcacccacgccaaccacctccctccccacgccccaggcccggctgcgcggcaacatcgccgacccctcctctccggagctgttgcacttccttttcgggcctctgcagatggtgagacccgccccaggccctcgggcccccctgcagcgggaggaatcgggttcgacttgtagaaggtgtggcggcacagcctgcccctcctgctcccctgacagattgtgaacacgtcgggggggccggagttcgcgagcagtgtgcggcggccgcatctgacatcggatgccgtggcgctgctgcgggacaacgtcactccacgtgaaaacgagctctggacctcgctgggggactcgtggacccgccccgggtgaggggcg

SEQ ID NO: 292 - DNA, 347 bp, Homo sapiens
  source 1..347: mol_type = genomic DNA
cgctcccctgcgtttcggctgctggcccagcagagggcgccggagagcggacggagccgcaggcggggttgtgggaagacgctgggatcccgtgcgcgcttgtgctccttccttctgggccagcgcgcagactatcaaccgaggtattcagtccctcggaaacccagttcctccgtttccaggcttctcgcccacacggagacagtcgctctcacacgtacctctcgcgggctcttactcgtggtcccggtctcctcctggtccccaccttccagcccagcgcgcacactagacggcacgtgctctccacgctctttctccagcgccccttgctcacccgccccccg

SEQ ID NO: 293 - DNA, 712 bp, Homo sapiens
  source 1..712: mol_type = genomic DNA
cgcagcaagcctcagccgcccttatttaagataccgcccgagagggcggggaaagaaaacccctacaggcaggacagcccttcgcccctcccaccaccactgcggcaaacaagccccgcccccagagggtagccgggcacgccggcgccgcgaggccgatggcaccccgcaggaaggggcattttcgcagcccccagacccagcccaggaggcccagggctcccgagagccgcattccgccgtggcaacagggatagcattggtgtcgaccttgctggacttgccgtggcagagcccccggctgtctgccctaatgcccccggtttgctgcggtggcccccggccagtcagaaaggcgcggagaccctgcagcagcccctcagaccccggactgcaagatggcggcaccaacgcagcagcccgggctgcgcccacccctgccccaaaaaggcaccaaccatccacagcctggccgccactgtgcccactctcggactgggcagcgggcggccaagtaccatgtccgccagcccgccctgccacaagggtcgctggcttgccgcggggtcttgggcttgcggcaagacggccactctggcccagagccgtcgcgacaccaaggtcccgcgggcaggaggcgcgcggggcggccgaaggcgcactcacctcacctcagtgctgcgcagcctcgggcacgaacagccgcctagcg

SEQ ID NO: 294 - DNA, 932 bp, Homo sapiens
  source 1..932: mol_type = genomic DNA
cgcccaacgaagaggagggagtaggggtccgcgccccaccacgaaaggagagtagagggctcgcgccctgcggggagaggcgttagggggtccgcgcccggcgaggagatgcgcgtaccagccccgcacgccaaggggagaagacacgcgggacggtccgcgcctggcggagagagttcctcacgcccgccccgccgggaaccacaagccagcgcccctaacaagcccctgcccagagcagggctgccgcgtaccctcacagacagaaagagcgaccacgcagccaccgcccagccccagccgctctggagtccttgaccccaccctcactaggccttggctccgcgaccggtgggcgggaacggaggaaacagacccgagaggccgcgagaggacggaactcacttcccgccgccgtagcgtcctcgtcagctcgccctccgactctccgcatgggcgcgcagagttcccggatgcgatattccggtgacaccggacgctgggggcggggcctagtgcgacgagggcggggccggggccggcccggggcggggccgtggccgcagctgcctggcagccagaacctgggagcgctcgcagtgctgcctttcggggttctttgtccttcgcttctgctggtaggtgaggtttaacagacggaaagacgccgggagcgcacgtgggctgccttgagccgctgcggacgagcaccttgtctctcaggttgaccagccgtatatccgcttgcgagcctgcaccaccctgggcgcgcgtggtctcggcagaatctcgttcacacgggtcccactcgtaggctgctgtgtctccttcctctcacggcgtcggggactggggcctggagtcactcctctacaggggtgtggaacgcgccaatggacgctgacaccttcatgcggtcgtttaacgctccg

SEQ ID NO: 295 - DNA, 422 bp, Homo sapiens
  source 1..422: mol_type = genomic DNA
cgcttccctatcggggctgcacgtagagcatctaaccgcatgttcccggaacctgggatgcgcgcgcgcaaaggagcgcccaagcgtgcaacccagagtttggacgcgccgccctttacagcccccgccccggggccgggcgagctgagggggcgcggggcgtctgtgttcccagctgatcacagaacaggccgatatcgcattgacccggggagctgaggtgaagggccgctgtggccacagccagtcggagctgcaagtgttctgggtggatcgcgcatatgcactcaaaatgctctttgtaaaggtaactccgagcccagcgggcagaggggccgcaggctccgcgtgggtgtgggaagtggcggcccctaggtttaagaacccagacgctgcggggacgattgaagcgcacctccccg

SEQ ID NO: 296 - DNA, 10782 bp, Homo sapiens
  source 1..10782: mol_type = genomic DNA
cgcggagacatctcgagtcccgcaggtgtctgagctgcgctgcgcgccccagccgggaagccttttcttatccaaccgcgccgccccgcggaaagcctgggtactgcagacgccctcaccggcccaccgacagctggttttgaagcgggggcttagaaaagtaaaggggacagtgtttggagccacgatgcctttgatgtgaggcggttccacccaacaatcaacgaatcaagctggaactgcaggccctacttttgttggggggaggaccttaaggaatgtcacccccttgtcccctaccctctgccccacgcccagcctctcagaaggcaccaggcgtgagtagagtgcgaggcgtccacaccctcgtgtcccttccattcccggacacctttgcaccctagccgaggcctcagcacggagtgagggttgcctggggtgccctgctgtgtgtggggcgttcttgcctcccgggccctgaccccacggtctgggtcggcgaggcgtctctccctggcgtctggactgtgcgcctgccccgcttccaggcgacgaacggcaggagccgggggtccggcgggacccggatccagggcgggcggaaggcgagggtctctccttcccggaacccccgccccggaaacctgcaaccaaaccgggtccctcctccgcatcgctcccgcgctcccgggccgcctccccacgcgggcacccactctccgggccctcgcagcaccgccctctccgcgtccacccgcgcggccgacgccgccaggggccctgagatcgcggagacagcgcgccctggcgcctggtggggccctcccagtggccagagcgctcctggaccagtcggctgtggtctgtgcccaaacacgatggaaaccggggctgggacgggcggtggtacgcacgtggaggagaagaggcatggcgggaaaaccacagccgaacacaggtcgactcgcacctcccaggcacccgccgccgtctgactcgcggctctccccaaggttccgactcgccgccacctcttggcaccactcaccgggctccgacccgggccgcgagtccgctgatgcgcagctgcctccggcggctcccgggctctgcccggacccgcgcgcagagacgctcgggaacgctgggctcgggcgcctcctggcggcggcagcacgcggcacacgctcggttctccggcccgagggggcgcgtggagcgtacgcgggatccgcgcgcgggcctgggctgctggcccacatttccccgcttgctcggatggcggcgctgaggcctattcgcctctttattaataacgatgcctcgggagggaaaaaatagagcctttaaaaatcagagtgcgagagaggagtgtgtgtgtgtgtgtgtgtgtgtgtgtgtgtgtgtgtctcggggcgttggtgaaagcaataaaactcagatcgcttccttggcgagatagcggcggctataatgcgccggggggatcaggggtttatttgcagatcctggataaaagatgatatatcgacacggtttagagtgtcactgcgcttggcctctcagggagccacaagtacatatttcctgaaataccgcccagcacagtatctaatccgcccgccacttaacaccgggaggggaacttcagcagcgcacaagcgcgctcccggtttcctggttctgggtggggagagcccaaatgagaaggatctggtatgcctagacgccccctcccctggcctttgtcttcagtccaagtaatcacttagcgatgggccttaataaatacgtctgagaactgtccttatgctcggtaaatattggattagcttttcaatgaccaggctcttgaaaaggctcgagaaatacacaccaggtctcaactaagcccaacagtcaataggtaaggaaataagttatttccccatctgaggtggggaatgttgggaagggggacttccactcttaccaaattatctgattctactaaatatgaccgttgtactaaataggaaagggggcgcatccaagtttcccattagtcggaatttccaccagctcagttctgctcattaagggccgagtgactttgtggggaacatatctgggttccagtttttatttccacagagctcagcataaactatttttgtttagtacatatggtttatttggggactgcttatgggagttttgacctggcaattgcaggggagaaggcatgacccaaattatttccgactgcctcgcgccaatttggaccgcttagaggaggcaaaaaatgggcaaattccttggaagccctgctgtgtctccctggtactgaattttgtaagcacgcgatacaggagacattgaaaattaaagaggaagtatctccctcctccaacatggggagagacgctgttggaagaagtttctgaagtggtttcccatcgcaccaagaggccagtgggatggatccccgcgcataattcaaaggaacgcgcgcgtctttagctcttcggggttggtaaccaagtggtccggccgggcgcaagtcgccttggaaccggctgcagcccaagtctagctgggcccgggactgagcggcacccttggcctctcagaacctttctcgcggtgagtggaagggcggctccgcgcgcgggcgcttcggggccggtcggttgcgaaatcagggggtggattttcatctacccaggagccgctaagcccgtaggggaccgagcatcacccaggccctgggtaccctgagttgcctcctgagccccaggacggtccgatggtgcagagctcccaggcagaagttagtttcctgatacacacacagcagtgagcaaagtgcgcacaagtgagccaaaccctgaagacgcaggttcgcggactcccatcccagcgttagagtcaaggagacagaagagaggaaggagagaagtgaggaagggagagagattatgagaaaaagagattatgaaagtagagaaagagattatgagagcgagattatgagaagagagaaaaagagagattttgagagagagagacagaatgagagagatccaactttcagtgtcacggggcctaggcggggcaagcgccctccgaagccatcaaagacaggggtggctttttctcccctcctccagcctcctccccaatagatatagcacgaatttttaatgcgtcttcctttcacagacaacaatacagggtttgaaagccggacacaatttaggttttgtgcgggtccctcctcgcgctccctcggcccagctggccatatggaatccagcatggagcccgtggatgacaaagggacccagtgacaggcccaacaaaaccccaaggagacttctgcctgactccgggattcactgccggcgataagcgcgctgaatggtgcggggtggggcggggggtggggggcgggccccttctccttcttcccctctctctgtccccgcgatcctttctctgtccctttttaatatgcacgcctctaacatctccggctcccagctttcccattgtgcggttttgtaccgcccgctgtagattgggcgaagatagactgctgaagcggaccccgcgccgggtgcgctcccagggcgctggggttagggccacacagggctccgcaggcgcttttattgactccatggggacccgaggacagagagaggcgggcgccgggtccacctcccgggctcccggccggccacaggagcgcacacccagattgcaggcccgcggtcctggccacagctccaaatttgtcctcaatgagcaagtggtggaagcctgccagggtgaggaccttccacgcttcccccttcctctctggaaacctgagggtctcttgtgatcattctcaccctggaagccctggggcctggaaaagtcaaaatggcctcaggccttctgacagctccgctccaagagacttgaaaccctcacctttcctcccaagaggattttgggattaggggaagttgcaagtggatgttgagtggttagtaccagggcaggttgggcgttcaattattgcacaacaaactatttgggagaaattcgttggcctttaaaaacagtaagacataagcccccaaacattgccaagctagtgtggagaacaaagggagaccttttagcatttagaaaaactgccctttccccgaggatgagcccggagatgggaaaggacctttactggggtattagaaagacttccgagccctcaattatccaggggcggtgtggaacgcgggaataaagtgtcatcgcgtccctgggctgctgcgcagagtgctatcgacgcacagatttggtcgcacggataatcatgtttgctttagatattgaattaaacctgatcatcaactacagggagcgagaaagtcagctgggaccgctccttttgcccggcctcctcactgccacccaaccccccagccttgatggtgcagcttcggagagaacagagatacaatttccaaatgcgccgacgttcagtccctcagcgtctgcagcacccgctgggctctgagctagagtcccaagtatttttagaaacccgcgagatagaggagaaaggctcatcctccagttggccaggcttcctaagacgctcccccgcgtggccaagtgtgcgccagcatctgccaaggagatcttcctccagcaacaagggtgggacggaatccgttccccgccggaaagatggatgcgcccttcccgggcgtgcccggggctgtgtgcccacttcccatccggggagctcaccccacccccaaacctctacttcctagcaatccacccagttccacgctcaagggctgacctggggggaccgcacaccctggttcggaccgccgaacgctggagggaaggaggcccgggatccagtccccggagagaggcctggaagcccggaaaggatgggagcgtaagggaagggagaggcatggtttgctgggattgccaaagattctcgctccctggcttcctggggccaaagatcctagggagggctgcagtatcccctccctcccccgagcccagaaccccgcgtacttcccccaagccccctaagccaaagcctcgcgcgccctgaccgttcccacccccagccttagcggtctcctaggtcagcaccagcccgcagattttgaaaaggagtggggagtggggagaaggcagccagtcttaacctcagacccccacccacccactttcttagcgtgaggtaagtcaaatgtaacaaagtgaaagtcatcgagtgtagtggcttcaacagcttctcaggctgaaggagccactcatagatttgggccagcgaggggggtttgctctcctcgcctttgcggctcccgaagtcacttttcctcccgtagccggacgctgccgcccggggttcagacagcgcgcgagaccaggggagatgcgggggagggtgggcggccctgggcttccggccgaccgctcgcaggccggaggccgagcgttctcctccccgccccaatcctaggcgcggggtgcagcctgtggtcggaggggcctgcaccgggagccccgcccgccccagggccccggtacccccggcctccccgcccgagaggaaccgcctgtgggagccccgcgggcggccgggcccggctcccaggcccaggcctccggcccggcgcagtaggacaaacgggagggcagggattttttctttttttcttttttaaacacaggatttttattaaaattcttatttaaaaaatcgaaagctttctgcgcccggcgctgttggggaaccgcgccgaatagctgagctccaagttcggggctgaaggcccggaggacagggcagagggctccctgcctacagggttttcttttccatatttgagaaatgtttgcacaataaaataaaaagagaaaggaagaaagcaggggaaaacgcaaaaacaaaaacaaaacaaaaaacaaacccaaacaagccacaaagaaaggagttggacccagacgggaaacaggccggtcctgaaggtcattttggcaacaatcaccaccgatatttacaacgaaaagcgaaatctgccaccagttgtcagaacgtttacatggccataaatatttagcattttcttatttttttttaaaaaaaggaagaaattctctgtatttttaaagtgttctccacttgctttagaagacggctgacaatatcgctactcacacaaacatattttacaaaatgcacgaaagcaggggtggggggtcggtctttttctcgttttcaagtgacgacattaacgctgggacggtttggtccccccggggagaggcaaagaagcgaagctgcgcaaacattctgtaaacacggcgtagagttcagccctctcccaaggttcagaaggagaggcatgggcagagctgggtgggtggaatctgccactccaaggagacgcaggcaacctcccggcgcctccctagtggagccgagagtcaactcgactccataataataattataataataataataaccaccataaggaccgaggcctcctcgccgccaccgccgccggggtggggcctgggcctggggccgcgagtctcgttggggcggcgctcaccaagtccactgctgggcctggaccagggggtgtgctgtcgggtactggggggtgctggccgagctgtactgggcgttgtactgcatgtgctgcagcgactgcgcgctgtaggcagaaaagggaatgcccgcctggaaggtggcggctgccaggtcctgggctttgagcgcgtgacatggtttgccgtccctgaccaagacgggcacggccacccggcgcggcgagggcaggggcgtcacctccatacctttctcggcccgggcgcgcttcatcttgtagcggtggttctggaaccagatcttgacctgcgtgggcgtgaggcggatgaggctggccaggtgttcgcgctcgggcgccgacaggtaccgctgctgccgaaagcgccgctccagctcgtaggtctgcgccttggagaaaagcactcgccgctttcgcttcttgccggcgtcccccccgccgcccggggtctccttgtcattgtccggtgactcgtcggccgagggctccggggacttggagcttgagtcctgagggggcgccccggcagccagaccgtgcactggggggagggggagagagaagcgcgtcaggcgtctggaggccgcgcgcagcctgcaccagactcggagcaccctggatcctccgtgcgaccctggacctccgcgcctggcagcccagccccgctgcctttgccatgacccctgccttcctctacgcctgacgaagacgtcgggacctcccagggtaaagagggaaatccgtaggagtgggggctaggggctcgcctttcaaggggctcacatagtgtgggtgagaatttggggggggaagtggcactgaagatcatcgcgggttaagggctccggcgcccttaagcggtttcctctccggcgagacgtgggcagatttttaaaagaaataccccacaatcccagcattgatcggctcggccatttaaggcaggtttttgggggctttcgtttctgctttcgggccttctagacaccccctgcactgcacaaagcgtcctctatgatcgcgcagggggcagggtgagctatttatacccgggccacccgaagcctccccaccctccacccgtacccggacgaggctcgctggctacctccagggcgccaggccagtccctcccagcggccggagtccgggggctgcggctggagccatcggtccgggttgacatcatataccagcccctgggaggtatagcccttctccctctttctccttaacttctcgcattgaaaaaaattttggagaccaagttctttcccggaactaaggaggcttgaagaggagggcagaggacatcctatacaggtgttaaaaatctttctacggatccgagtgagggggtccgggcttacatggccccttcccctttcactcccagcgtccaacccgggctgcggctgcaggaatggaggggaccacggcctcggcagccaagttttgctacttacgggagtactgaaggccctcggtgctggccagccagcgcgtgtacgggttgtcgctgctgtcgtagaaggggttcttcaggggcaggctctgcaccgcgtccagggcgccctgccccagcggcccggccctcttggctggctcgggcccctcgttctcttcctccggaccttcggccacagagccctcctcatcgttggtgtccggcaggtctaagatgtccttgaccgaaaaccccgtctttgtgttggtcagcgacatggttcgagaccccaaaatttatgtcgcaaagttgtagcttcacttggtcaattcgtggcgctcccctgccccggcgggcgggggaggggggagttggggggagggactgggggaggggaggggggaattggctttaattattgggataattattatttttaaaaagagaaagaaactggggatggggaggaaaaaaatgaagcccaacccagtgcctctctctgtcttctttgaaagcacgcggaaatggacgcaggaagccgggcggcctgcgcgccgagcgccgcgggccccggccttagtttctaactccaggaggggtgccaaggcggcgtcagctcgggctccggcggccgctcggcgcgcggtggcggctggtggcgaggaaaaaatgggccattgcccgagcgatcagtccatataaggctgggctccactcacgaacctggggaggcggggagagggggagaaagagagggagggagggaaagaaagagggagggagggggagcgagagagggagcgggagaagggtggaaaaaagggggaagagacattaaaaacgcaaaggttggccacgtgtgggcgggtcttgggagtcaagtggatgaagacagtatttgcagatgtgaaattgtgggttttggggagctccgcgctcccagccaacggccctctagagcaagatgagaggtgtaacgtgtcaattaattgcaaagacggggcgagcctttttttttttaaacccagtatttacatacaaagggccaccgcgtcgctcgcgagtccacacacttgaaagggccgttttaacaaattgcatcttaaaaaaggtgggggtggggaggaggaggggagaaaacaaaacagaaaccaggaggagggaaaaaatcctctttaacattcaccggttcctacctccccgcccccgcgcgcccacccccggcaagccggaaaattggcgatttgtggcgcctttggaaaagggggagggggcgaaggccgagctgcggaaatctctcctttccatcgttggtggttgcctaaacaagatccggttcaaatggcaagagcccaacttctgtaagcctcctaggtcaatattttggttgaggcttaaggatgagtgctagaaatgacaaggcaagtaattgattccagttaaccgcgagagagcccaaaacccaggagagagctccgctccgggccggccagagccaccaccaagcgacccgcccccttccagccgccctttatttgctaaagactcatttatttccttcactccgcctccccccacctcccacccccaaccccccgcctccacctctctgcggccaaggaggacgcgcagttcactttctgctttgcgggcggtcccggggagggcggggtaagagtgaggagctccaacttcgtctggggtcggaccctggccctgctgcctctgggctctgcgggcctctttccccgggtggaggaaagggcagacggccctgctctttcctgccctctattatccctccctctccggccccctccctctccagcctctggctagaggtggagcagcgggaaggcggcggggcgggattgcgggggtgggggtgggggttgtgtctccagcctgggtgaagacccagaaagctgcgggggaggggggtcgctgggtggcccgacgatgcctcgttttctccccagttttcttctcccacgcgagttctttagtgtggtctctttttggacgccttgggtgcgttcggagtacgcgctccacgtttccaggttaagtcaggttagtgtgaagtttccagccactctcggctctgggaactgaactttcccgggacttgcacttcctcctcggcgctggggttggaaaagaccacgccgcatttgccaccccttcttctagttaaaacttttttggggggggggtgcaggggtgtagattggctacctgatgctcgttcgccccagaggagtgtcaggaaggagctttgtctgggggcggggaggggcacagaaggccacccctgcggggtccaggtctagtcttcccacaacctgggctcctccgcgcgggccaagctgagcgcgcgggcaccgggtcctcctcgcgcgcagctgcagcggacgccgccgcgccccagctgcccccgggttgccagcgctgggtgagggcacctctccccgagccgctcggcggctttctgcagccctagctgcctcgcccccagtatgtgacgtgggtgacaatggcccaggttagagcaagcccctcgtcggcgcgtcctgggtggtcggacccgggcaaacacaaatacaaaccgattgctaagctgcggacaatgagcgaaatgtagacaaatgtcccgctcccgttggaagcctttgtcccggctcg

SEQ ID NO: 297 - DNA, 616 bp, Homo sapiens
  source 1..616: mol_type = genomic DNA
cgccacggcgtggccaccgaggtggtgtaccgctgcgacacctgcggccagaccttcgccaaccgctgcaacctgaagagccaccagcgccacgtgcacagcagcgagcgccatttcccatgcgagctgtgcgggaagaagttcaagcgcaagaaggacgtgaagcggcacgtgctgcaggtgcatgagggcggcggcgagcggcaccgctgcggccagtgcggcaagggcctgagttccaagacagcgctgcggctgcacgagcgcacacacacgggagaccggccctacggctgcaccgagtgcggcgccaggttctcgcagccgtccgcgctcaagacgcacatgaggtacgcggggagccgtccggaggggtccctgcgcactggaagggtgtgtcaagcactgaggtcgtcctgggatttgatgtctcactgcttaatttctccgctccaaacctgggggcttatttcgagacagcgtttagttctctgtcaaggtacaggggctgcgagtgatggctctgctcccttgttctgagacccctggggcgcccctgctggccgggacgtccctctggtgggaccgcgacagcggccgcgggctcgccccaccg

SEQ ID NO: 298 - DNA, 1687 bp, Homo sapiens
  source 1..1687: mol_type = genomic DNA
cgcgctttgaacccaaccagccctcatcgctcggagccctgcgtcccccgcttcccaatcccagaaccccggaagcagatctcctcagtcccactccctgagggtcctacagtccgaggtcccgcagcgagggaggagcaaacgcctgagctccgcgggaacccggcgcggctgcctgcgcgcacgcagggaccggagcccgagcctgggccctgcaaagtgggtctcccactcgctgcgcgttcgagtggggcctccgctaaacatgcagattaaacaaggggcttccgataaggataattacaggaggctgcgcattgactttccgttactaaaggaagacccaaatctcccccttgcaagctgttcccgggattaccaaaacaatcactttcttttgagatcattgtcttttgaaccactccagctcggggcagaacgctttagacgctggcgttgttttccgctccgcgaggaattttcccccagatttatctctgggtttagggaaggcgttgggcattcgggactgcgtagtcgtttgtaaatccgaatttggggtagcagttaagtgagatacctacgcttccacacagtttgtagcccgagaccagtcaggcaggcagggtagtcgattctaaggtggcccgaccggggtcgcccgcgaatggaaagcctaaggggcgagggcggacgcgggcgccgtgcctgcagccccgggtgccgaccaagtggcctcacctgggacaggtcttggggtctccctcccctaggtctccctggaaggtaggcgatcacgggagtgacagaccccgagtagatagccagtgatccaaagacaccaaaaggccaggcggtaggagcccagcctcagacactctctatagtgaccgcggcgcctcgggaaggatcgtcttaaactcattttgcaggtgagagcaactgggacttattaacccagtcacatcggacacagagacgtggcgagggcgacacagagcaggagactcaatcaaggagcccgtattggagctctaaggggactcagagatgcagaaggaacgaacttctaaggggtcttgcaattcctggactggggcgttcctaatgcatcccgggaccccaatgccagggaggggcctgcaggaccccagcggtgggcgagttgtgtcctgggtcaccttgtgtttcgcagcgtggcggtggcaggagcccagcgcgggaggacattttcatagcctcctacagtgagaaacgccccccacccgacgctgcgctcatctgtgtccccgctgttgccggggctctggtatccacttgcgcgccctatgtggtggggatccacccagagcccagcgtcaagttatacgggcgcttcactcagcgtcagccaagaccagggaagcgcttcttgccgtttaggagacgtctgcaagagataaaaagctagcccacgatccacccacaatcctcgtgtccccggggtgccctcgcagttgccaaacctacgggccgcgtttaggggaagcctccgcgtcctggcggccaaaagaatgggctccttccagcttccccctaccggataccacctgcaaatctattgccagaggcgcagctcccgcgaacgttcccgccccgggcggcccccaggagtcgttttgggtccctaaatccgttgagggttccg

SEQ ID NO: 299 - DNA, 5242 bp, Homo sapiens
  source 1..5242: mol_type = genomic DNA
cggccgtcggagcagggccccagcgcaccgcgagcctctgcttcctccgccttgcagcgccgcgagcccttctttcctccgcgcctggccccgtctgcgcccctctcggcgtgggcattcgcagacccgctaggaggctgacgagaggtgtcctcatagagtcctgccttcttccagacccccaaacgaaaagaaagagaaaagccaacctttcgcctccattctgcacgcatttggagagcggctggtggcgagcgatccgcacaatcgcttccctcgcggcgtccctggacgggcggagagaacccgggtgactgagcctgaggctgagcttccttgggagcgatccatgccagccacttctaccttcaagggggactcgcggccgggtttacgcgcgaaggatgggtgccagggaaactgacttatagacgacccgctccgcgagagactgacaactcagtcctgcgggctttcggggcgcccagaccagtccttagccagaggatctggggctgcacagctcccggaccctggagggggtggtccgggggtgggagactgcgagcatcagcccacgccggccccctcccaccctgcggcggcgaggtgggaagggccaggatgcgagcttagcacctcccctagaaatgcagcacttcgcccccccacctcccctgccctcggcgctccctctttcacgcgctccctcccgcctccttcccaagggcgcccttcttctgcccccagctcacgtctgaatccctcggcgccccctttctcttctcctagccccttcctcacgtcccctgcctccgggtatttcctctctccaatcccccacccccgcaccgcctgattccgaggggcgggagcgcattgggctgcgcacgggtgggggcgccgcgccagcttcgcgtagctgctctgacgccgctgccgccgccgccgccgccgccgccctccgcagcccagctcgcgccccgcggcagctccgcagtgcactagccaccaccgccgccgccgccgctccgccagacctgctgccagcttgcccggtccagccctgagagagcctcgaacgccagctgcgagggtcatgagccagagagccccggggcgccgcgcggagagcaagcggagatagcgactttgcgccccccagccctcgccttcttgcatcgcgttccccgcatcctcgggtccttctgtcctttccgctgtccccaccgccgccatggccaccttgctccgcagcaagctgtccaacgtggccacgtccgtgtccaacaagtcccaggccaagatgagcggcatgttcgccaggatgggttttcaggcggccacggatgaggaggcggtgggcttcgcgcattgcgacgacctcgactttgagcaccgccagggcctgcagatggacatcctgaaagccgagggagagccctgcggggacgagggcgctgaagcgcccgtcgagggagacatccattatcagcgaggcagcggagctcctctgccgccctccggctccaaggaccaggtgggaggtggtggcgaattcgggggccacgacaagcccaaaatcacggcgtgggaggcaggctggaacgtgaccaacgccatccaggtaagcgcgggattcccagttctgcctgtcctcccccctcccagctcagcgtgccgggctctgcccccgacagtcgcccggtgatctcggcctggagaccccctcctgtacccaggaatctccctttctccatccctcccagccctgcgcggggacctacgcccccaggcggtgttttccgccctaacccacgctccctcccaacggcaccagctgcaagaccgctaggctgaagttcggtctgagacacctgtccggagacactgcaaaagtgaaggaaatggggggagggagcaggaagcgatgagaaagaaagaaaatcaggattggagggcacggtttggtcttggactctggaacggattcacagctgcatttttgggaggaaagaagaaggggaaatcgctgaggtcggagtctcctccccccgcacacacgcatagacacgcacacgtatataggcaggcacaccacatgcccacagccccccttcccacgaacaaaggcgcaggcacaggcgcacacgacatgtatctatatatagaccacagtactatccccaaatgcacctgcgtacacccctacagctccacaaacaaaagcatatacctgcacataccaaacacacgaatattcgtttgaacatatgcacacacacatgcacagatgcatgcagacaggaaccagcctgctcccttgggtgcggccctgttggagacatacaggtatacacacacgaaagcacacacatgctcccacacaaacacatttagtccgcaataatccagcattacctgtgcacagactcgcccctctcgcccgaaatcccggctgcaccgcaaagaggcggccagctcctccccgtgtgaccctggcgaagccctctccagaggctaagaatggcccctgctctctaggccctctccccttattcctaaccccccactccccaccaggcggcgcaggactcctacatagggagcaggtttggagtcttggcaacgacctccgagctagcaggggctggcaggccgcagctttctgggccgaaggagacttcagctctaggcaaccccggtcccttacagcttggccccgcggcgtccctggagcgcactatcactggggccacggaaggcaggttttctgggagcagaggcctcccaggggttgttccatgtatcggggtaagcaggactccaccgggccccgaacaccagatggcccgacccaaggcgctttccgctccggggcccggcgagacggccttgctggatcacggacccggggctgctcccgccttcgctgtgccccgcgacttcccggggcgtctttcaaggctccgtttgataggccccaagggaggccagggcaggaggctggaggctcgtaggcctccgggaccctggatttcgttagctcttagtccccgtgtggattctaaaccctacagccctgtccgcctgaccctgaagcttctggttctagactcttgctcggttcggctttctggcctccctggcgccttcccgccaagctcctcctcctgcttgcctccccagcctccacttagtccccttccttaggtctcgccctcgcctcactctaactccagccttgtcctaagactctcaatttccagccctgcttcctcctccccgcgcgctgctccccaagcgtcttcacattgtctctgttcttgcctcccaggggctttgccccaccttcagcccctctccctctctaccctatttccttttcgcctcacaactgcagcctttaacgtttcctcctctggtctgagcctgagacgcccgagttttcctctttagctcccgtgccccattccagccccaccacaaatcccgtgcccactctttccactggcccaggcccagctcactacctttcttagagcccctttccgactacgctcccttctgtcccttctcatctcctcccccaactcctcattctgttcctgtccccagaaaccatcccgaagtatctcccttccctcctgtggcttctccctattctcagccctctcccaccctagccctctcgctatcaccagccattagcgcacctttccttctcagatccgtccatacatccctccctctccatctctggctcgacacccgggctcactattagttcccctacatccaccccgcagcctgctcttaacctctcctccccggcggctcagacccaattctcagtgtccttagcgcccccttcgggccacagcgttaagccacgccccccgggcccctcatccgttgccaagttcgctgagcgtccgcgtctggttgcctctccgccccacagggcatgttcgtgctgggcctaccctacgccatcctgcacggcggctacctggggttgtttctcatcatcttcgccgccgttgtgtgctgctacaccggcaagatcctcatcgcgtgcctgtacgaggagaatgaagacggcgaggtggtgcgcgtgcgggactcgtacgtggccatagccaacgcctgctgcgccccgcgcttcccaacgctgggcggccgagtggtgaacgtagcgcagatcatcgagctggtgatgacgtgcatcctgtacgtggtggtgagtggcaacctcatgtacaacagcttcccggggctgcccgtgtcgcagaagtcctggtccattatcgccacggccgtgctgctgccttgcgccttccttaagaacctcaaggccgtgtccaagttcagtctgctgtgcactctggcccacttcgtcatcaatatcctggtcatagcctactgtctatcgcgggcgcgcgactgggcctgggagaaggtcaagttctacatcgacgtcaagaagttccccatctccattggcatcatcgtgttcagctacacgtctcagatcttcctgccttcgctggagggcaatatgcagcagcccagcgagttccactgcatgatgaactggacgcacatcgcagcctgcgtgctcaagggcctcttcgcgctcgtcgcctacctcacctgggccgacgagaccaaggaggtcatcacggataacctgcccggctccatccgcgccgtggtcaacatctttctggtggccaaggcgctgttgtcctatcctctgccattctttgccgctgtcgaggtgctggagaagtcgctcttccaggaaggcagccgcgcctttttcccggcctgctacagcggcgacgggcgcctgaagtcctgggggctgacgctgcgctgcgcgctcgtcgtcttcacgctgctcatggccatttatgtgccgcacttcgcgctgctcatgggcctcaccggcagcctcacgggcgccggcctctgtttcttgctgcccagcctctttcacctgcgcctgctctggcgcaagctgctgtggcaccaagtcttcttcgacgtcgccatcttcgtcatcggcggcatctgcagcgtgtccggcttcgtgcactccctcgagggcctcatcgaagcctaccgaaccaacgcggaggactagggcgcaagggcgagcccccgccgcgcttctgcgctctctcccttctcccctcaccccgcccccaccagcccagtgcgccctgccgccgcg

SEQ ID NO: 300 - DNA, 3437 bp, Homo sapiens
  source 1..3437: mol_type = genomic DNA
cgctggtggagcccgcctccctgaagccggagttggcgagtttctcgcacttgaccttgtaggcgtctctctcgcgggccagccgggacacctcctgcttaagctgctccacctgctgaatgagctgcgtcttctcattctccaggtggtgcttctgctggacgcgtttatacctgcaagactgggcgtagccccggttcttcagggtccgccgcttctgcttcaggcggatcacctcgtccttggtgaagccccgcaggtggcggttcagctcgcgcacggacatggacacgagctggtcgtcggagaagcggtcctccacgctgccgttgccgcccgccgccgtcgccgaggccgtcgcgtgcggcccgggcccggggtggctagtgggcagctgttgcgccgggctagcggcgctggacggcggcggcgacgcttggtgatgatggtgatggtgcgggtgagcgtgcgggcccagctcgtcgtgggccacgccggcgcccgggtacgcgtggtgcgggtgagggtggtggtgatggtggtggtggtgagcgccgcgaaagctgtcgaagctttgcagcggctgtggcactgggtgcgagccgatgagcgcttccaccgcgtcctcgggcgtcaggttgagcgcctcggggttcatctgctggtagttgctcgccatccagtacagatcctcgaggtgtgtcttctgttcggtcgggctgaagctgggcgacgagggcacggagctacacggagtgctgagcggtgtggaggacaccgagccggctggctgcaggcgtgtgcagggcctgcccggacgctccgcgcgccccagtggctccttcttcacgtcgaacttgagcaggtcgaagtcgttgacatactccatggccagcgggctggtgggcagctctggccccatgctcagctccgcggccatcgctgaagcgaggcgcagccgccgctgccgcccgggaaactttgcggccggccggagcgcgccgagccaagcgcgggggggaagagcggagaagagctggggaggcggggagcgagggcgcagcgggccggggccgccggccaagcctttgtctggggacgcggcggcgcgccggagagtcccgaggctgcctgcaccgccccagagctctgggctgtgcccgcgcagggaccgggccgggtagagtcgggcggggtggagaggcaagcggagcgcgcggtggggctgaggggaggcgtggggcgagtgcccgttgctcgctctctagctctcttgctcttacgctctctcgctcgcagccgctcgcagctcggcggtgcagctgtgctggatccggcggcgccgcagccttttatcgcctcctgatgtcactggggtgcgggggcccgggcggcccggtgcgcgggccaatagctgcacggcctccgcggcccagcggcgcagggcggggcgcgcctgacagctcccccgccccccgcgtcagctgactggcggcccgagcggccccggagcggcggaggcctggcggagcgctggagcggagtgggacggccagcctgggcccacccccgtaccctgcaggtcccggcccacgcacgctcgcctggagtgcgcgccccacctctaggccaaatcaccgctttcccctcctcgcgcactctcctccctcagttccctttgcaccccacccccatcccgtgtcacccccaaggaggctcagaatgagcgccgggacaacgcctcctgggccctttgttcccaagcggcccccgcccagtgggcgacgctctgtgtgtcctcgcggcttctggccgtgtgtgtcgtgcgttcctgtttctggagatctgcgcgtatttgtatgttggggagggcgggctcgaggctccgagagttgtgttcagacccaactcttaacctcaggggacctttctcaggccaagcgagggcccctcctggcgggtgcagtcgcagagccctgaggttcgactccactggccccgccgctccccgcgttcaccccaccgcacaatgttcacagtgaaggcgacgggaaaagcagcagcccaaaggctctgaattcctcttccccgccacacgcacggaatcctgagcccccggagcctcggggccgaggccggcccgggacggtgctccgagtagctctccactgctggggagccggccctgtttttgtttgaacgttttgtaacgattaagcagatcccggcgtcagcccgccgcggagaggctcaaacaggcataaagtgcgaccccaagtggccactgtgcgcaaaggcgccgcgaccgcccggcccacggccggaaggcttggacggcgcctcgtacccagccaggtctcccctacctggcccaacccaagccagcccagaacgcatactatgtgtgcaccagagcccaggacaggttcccctcgagcgatgtacaggtcctcgggtcccgtcttcgtactcagccgcgagcctcgagccgcgagctccgctctggtcgccccgttgaaattccgtgccccagcgttcgggggtgcccgtcggctgctccctgggccggaaggtcctgggcggaggaaggccggtagccaaaagtggaagcgccacagtgaagcggcccagggccaccgggtgagaaacctccccggagggcagacggggagaccgaagcacaccgcactaggcatccagactgggcttgggagccgcgcaccctccctacccagatccaggatggctagaattaacgggttctttctgagacctcggctcaggcgccgaaaccggatagatcgcgaattcgctggacccggagacccgacccgcctcccgcgtcaccttcttctttctagctttgggcgcgcgcagcgaaaggcaggagaggcgcgcactgggtgagtgagtcccggccgctgtctgcgctggaccagcccgactgacctcgcgcgtaggggtcgcgtgagccacaccggtgcagacgcgcctagattatttttaaatgttagaaggtaaaatatttgcctccaattaatctgaaaactctctattctcttgcgccctcggagaggctggggtacggcgtggtattgggccgcctatttttaataaaatgagtgtattttaactaaaacttaactcaatcttgtggggtggcaaattaaatgctggaagagcgcgtctacaaccctcttcgagaagcgtgctctccgcagaaatgagtcggccgcctggagagagagcctgggcggtgccgctgcgcagcccctgccagtagctgggggttggggactcgcaccttgtaaatgtcctcgtcttgtttgaacgcagtgagagcacactcgtttccagatcactcgggaccgggtgtctcggatctgtgcagactatgtatggctccggcctcaggcggccagggcgggacaagcacg

SEQ ID NO: 301 - DNA, 661 bp, Homo sapiens
  source 1..661: mol_type = genomic DNA
cgctggcaccaccgagccccgccgcaccccgccgctattctcaggacgacctgggcagccccccagcttctagaaaagacacagaggagactgcgcaatcaacaggcaggagtgtgcgtgagtgcggcatgacatgggcacgccaccccagagaagtgggcgcctgctgcgctaaacgtcatccgtcatcctttcgctgaaaagttggaaaacaaacaacaaaaaccccgcccagcctcatttaatgcgaaccaaacacaaaagccacaggggaaagcttttcctctctgtgcttcccttttccggccgaggaacaccaccagccaagataaggggactgtgcggggccctctgcgcggcttcaaagggctggcacccgcccgggcccgctcccactcccactggctaatgggctccccgctccagcccttggggagcaggaggcggccggggctccatgccaggcgacttggaagaggtttaaggctcacgccacacaagggaggaagctcggcctgtcaagaggcgtgataaagcagccccttatcgcagttttcctttcaactgcctccaagtgagcgccggccggggctgggcctgtcaaggccaagcgtgttcctttcatgtcgccctgggttcctcggtcagcgacgtatccacg

SEQ ID NO: 302 - DNA, 247 bp, Homo sapiens
  source 1..247: mol_type = genomic DNA
cgcgggtttaagtggacgccaggtgcaccggacacaggctgtgtttaaccacaccgaggactatgtgttgctgcccgacgagaggacgatcagtctttgctgctgggactcgaggacagccgagcggagaaacctgctgtcgttggggcacaacaatattgtacgctgcatagtgcactcccccaccaaccccgggttcatgacgtgcagcgatgacttcagagcgcggttttggtaccggagatcg

SEQ ID NO: 303 - DNA, 402 bp, Homo sapiens
  source 1..402: mol_type = genomic DNA
cgcacgcgcacgtgtgtgcctgcatgcactcacatgtgtgtttccaaccggccgacagtgacgcacgtatgccctcgccactcggcaagcacctcgtgctccttggtaaccagcagatgtttctctcaattactgattttcaagagcctggtgtttcctgacatgagccaggatggcgggcggcgcccaggggaggggaagtgaaccggggctccgtttccaaggctcttggacaaaaagcgaagccctggatcaggaagcacaatgcggctggtatttagccgcttctcaatagtgcctcattcaatccccggggcctcattgatgcccgccgcgtgctggagagcctctgagggtcagtcggcgacgggcaccgtctggaggggtctcgccgctaacccg

SEQ ID NO: 304 - DNA, 318 bp, Homo sapiens
  source 1..318: mol_type = genomic DNA
cgcagcatggtagcgggagcgcagctgcgcgggcaaccgccgggtttgtggccggttggcgggctcagtgccgcctcagtgggctctagcagtagaaaagtaaagcgactaccctaagatacccagagcgagaggagaggggctagaagggtgctggaggcaggctgtagtggggctaaaggagctgactgaccagcgctgcggccggtgcccgaagcagcgagcagcgcacctcagctccggttgccgcgcggcatggtatttatctgtgggttcagtgcgcacagcgttgtccagtctcgaaaccaccctaaaccg

SEQ ID NO: 305 - DNA, 215 bp, Homo sapiens
  source 1..215: mol_type = genomic DNA
cgtgagtgagggcgtgcacggtgtggggtcctgtgagtgcgggtgtgcacggtgtggggtcgtaagttcaggcgtgcacggtatggggttgtgtgagtgcatggcgtggggtcttgtgagtgcgggcgtgcacggtgtggggtcttgagttcaggcgtgcacggtatggggtcgtgtgagtgcatggtgtggggtcgtcagtgcgggcatgcacg

SEQ ID NO: 306 - DNA, 214 bp, Homo sapiens
  source 1..214: mol_type = genomic DNA
cgcagggcccacgctcccggcctctgggacgctcacggcagccccacgagaatgatcacacgagggcccacgctcccggcctcggggacgctcacggcagccccacaagaacgatcacacgagggcccatgctcccagccttggggacgctcatggcagccccacgagaatgatcacacgagggcccacgctcccggcctcggggacgctcacg

SEQ ID NO: 307 - DNA, 291 bp, Homo sapiens
  source 1..291: mol_type = genomic DNA
cgcggcctagagcggtgagtggggtctcgagcgcatcccgggtgtttgtgccgaggctggtgacgtccgaggtggcctctgagtgtgctgacttgtgaccctgagctgttgggggctcaccggtgactccatggtcttgttgagcaccctgcacgtggggctcagggtcggtaaaatagcagtgcgtggagaccgcgtgctagaggccgtggcgcccgcgtacaatgagtcgcagacagcacagacgggagtagggcagaatagacaatatcccgtgaattgcgtggggcg

SEQ ID NO: 308 - DNA, 568 bp, Homo sapiens
  source 1..568: mol_type = genomic DNA
cgccagcgcacagcgcggcaggacgcgcccgggtctcagcggacttgtgcatgttagctgtgtagatttatgtgagggcttgtaaaactctggtcttgtaaactagtcttaagcgcttttaatatggagacagatgagagcccctctccgctcccgtgtgggcccgcaggagaagcggtgatggagagccgagctcgccccttccaagcgctgccccgtgagcagtctccaccacctcccctgcaaacgtccagtggtgcagaggtaatggacgttggctctggtggtgatggacagtccgaactccctgctgaggaccccttcaacttctacggagcttctcttctctccaaaggatccttctctaagggccgcctcctcatagacccgaactgtagtggccacagcccgcgcaccgcccggcacgcacctgcggtccggaagttctcccctgaccttaagttgcttaaggatgtaaagattagcgtgagctttaccgagagctgcaggagtaaggacaggaaggtgctgtacacaggagcagagcgcgacgtgcgggcggagtgcg

SEQ ID NO: 309 - DNA, 386 bp, Homo sapiens
  source 1..386: mol_type = genomic DNA
cgtgcggggctggggcggcggttacctgggcgtcctggtagccctggagcagcaggaagtaggggcggttgctgggggcctggatgaggcactgagtcaactgatcgaagtccccggggtctgcagttccgatttgggcgtcggctgcccctggggccatgctaagtgcctgctgtctccgctcctgctgccgccgccgccgcccctgaaggctaagctccgacacgctgcgccgcaaagacaagttttctgagcgctccttgcctccagacccagctggggcccctgatccggtccccgggccaggactggccagcgctgccccacccgacgccgcccgggagcggttcttctgtggccgccacgaaggggcgccggtgcctgcg

SEQ ID NO: 310 - DNA, 611 bp, Homo sapiens
  source 1..611: mol_type = genomic DNA
cgaacgttaagtccgtccccagcgcttggaatcctacggcccccacagccggatcccctcagccttccaggtcctcaactcccgtggacgctgaacaatggcctccatggggctacaggtaatgggcatcgcgctggccgtcctgggctggctggccgtcatgctgtgctgcgcgctgcccatgtggcgcgtgacggccttcatcggcagcaacattgtcacctcgcagaccatctgggagggcctatggatgaactgcgtggtgcagagcaccggccagatgcagtgcaaggtgtacgactcgctgctggcactgccgcaggacctgcaggcggcccgcgccctcgtcatcatcagcatcatcgtggctgctctgggcgtgctgctgtccgtggtggggggcaagtgtaccaactgcctggaggatgaaagcgccaaggccaagaccatgatcgtggcgggcgtggtgttcctgttggccggccttatggtgatagtgccggtgtcctggacggcccacaacatcatccaagacttctacaatccgctggtggcctccgggcagaagcgggagatgggtgcctcgctctacgtcggctgggccgcctccg

SEQ ID NO: 311 - DNA, 5799 bp, Homo sapiens
  source 1..5799: mol_type = genomic DNA
cgccaacgtcttgtcgtcgtccgcgctgtccaccagggccttgtcagcgggcatgtaccaggcagcgctgtgctctgccatcagcgagtccaggtcaatggtgctcatggcgctcttgaccgcctcggagcagcagctgcccagctcgctgtcgccattctgcgcccctgaggccccgacggcgcactcacccttcttgccacccttcagccccagaggctggtcctcggagatgctgaactgctgcctctgtagctggatctgcgcctgaaggatctccagggggtggatctcgtcgggtggcggggcgccgctgctgctcgtcggggtgcggacctgctccaggcccggagtgcccggatggcccgggcccccaccgccgccgtagctgtcaggggtcgaggtagagttagacatgatgcccaggccgagggcgggcgggggcgccttcggtccgtgttccccggcgcctaccccacggggagggagtttgggcgagccggtcaccaggggactcctgctcgctttaactagtgcctgggggttgtcagagctggacgacacctcgtcctcattggcgtagctcgtgctcacctcgtccgaggcgaactcacccacgtttggcgaactactgtccgacttggccccgccgtccagggacccaatgaggtccggctgatcccccaggagcaactcagcccccttcccccaggatggcgacgtgagcgccttttcgtggggcgtcggtgccccgcgcgtctcgcctgcggagcttcccccgacggctgcgcctgacgcttgctgctgccctgggctcaccccaggtgcgcccccgctgtccggagccgccgagtacttgtcaaagaaggtgccagggctcacgtgaccactgtccctttttctgcgaccccgtccccggccgccgcccccggagaccggcttgccgtcattccccgacgtggattccagggtgtagttgggggagaggctggtgccgtccccctgggctggagggttgggcggccccgaggctttggagccgctgctactggtcccggacgggcctccgggtcctggggccccaggagcagtccctcctgggaagtaatccgagcccgggttgccggccactgccgcgccgtcggtctcgttctggctcagtttcctcttgccctctggcgggttcttcttgttgaaggtcacgttgaggttgggggccccgaggctggcgatcatgttctggcaagcggtggagagcgcagccaggcagctctggccgaacaggttgtccttggagctgggcttgttgaaggagcccagcgagagcgcgcccaatttactggccgaggtgcgctggctgggctggaaatcaggctgcggcgggtaggcacccccgccaccgccgccaccagagctgccaccgccccctcccgcgctggggggcgagttcacgcctggaccgctgtgcggcgtggactgccggccggctgcaccaaacggaaagcccggctggcccccgagcgcagacgtagcaaagtccggcggcgggggccggcgctccgaagcagcgctggggagccccacgcccgccccgggcgactgcagctgacccaggcctcccagactgcccccgaactgcaggcccggtgaaggcagcgcgggcacgtggccctctccgggcatcctcatcggctcctgcagagggccccggaacagcacccccgagccaccaggcggaggagggggcgccaggctggggtcgtgcgggccacagtcagcgggcagacccgagccgcccatcctacggggcagcaggtctccgggcggcggatgcggacctgagaaccacgcgctctcttgcgccaagtgcggcgcctgctgctcgaaggtgcccagacgcccggcgcccgtgctgccgccttcgcgctcaaagttcggctgggccaagccgcccaccgggccgccatgcaccaggccgccctggcccacgtccccggggtggcctagctgagccaggttgggctggcgcagccgctgctgctgattccgcgacgccatctgcttaatcatgagggccgcgttttggcgctgctgctgctgctgctgttgctgttgctgttgctgctgctgctgctgctgttgctgctgctgctgctgctgctgctgctgttgcagggactggtggtccggggccggatgctgcaggggcggccccgaagggaagctgtcgggcacaggcggtgtgaactcgccgggtaggcctgggtaggcggaaggggagaggtgattatccagagcgccgttgtgcatgctgccgttccacgaagcgcagcggtccactcccgcgctgcccggaaagtcgaagcgcggcctcttggccacgttcatgtaggggggcgcgtcgaaatgctgcagccgctggttgggcgcctgctgcggaggaggatgctgcatgctgaaaacaggctcggaataagggtgcatgctccggttctccagccggtggatgggatactcgaattgcgcgtgctggctgggcagcatggggcctccgtcctgcaggccgccgctgggcgtgcccgcctcgccctgctggggccgagggagcgcaggcgggcacgaattttgtcggactagaagcccgggtggcggcggcggctgctgctgtggcggctgctgcgggggctgctgctgagggggtggcggggcctgctggggaggctgcattaacgggtgcctggagcccactgagggctccagacccacaggcatctttctggccccactgaacctctcaaagaacacaccatgctgctgctgctgctgctggggctgctgctgctgctggggctgctgctgcggtggctgggcgtgcattttggacaagcccaccatgcccgcagctctgggcatggccgaggcgcccgggaaagcgcccccaggaacctggcgacccgctgcataatgaggcagctgcccttcggagtcagagggcgaaaacatgtcaaaatgtcccgagggcgcctcgcccgggtaattgtattccagcgagtcgacggctccttggttcgtcaccctccgtggctccagactgtgggaatcggagccgctggaggacggcaggccgtggaaggaggcggctcggttagggctctggtccagcggcaggcatggggccggcacggcgtggctggaggcacctgaactgtggaagtccgggaggttccccggtcgctgcgggccgaagctctcaggcccctggctctccgccatgtgctcatagccctcggcgaagggcggctggctgcccaggcctccggctgcgccgccgtagccgagcaggcgacccccgtgcaggcacgaggccccggggtccgggccaccgaagttgcccccaaagtgggggtgatgctggtggggatgatgacttcccgggtggccgtggtgaggctgctggccgccaaagaagccgtgcacaggctgcgcttgcagcccccctgcgtgcaactccgagtggccgcgcgcgtggaagccgtagggctccatgttcatgcccaagatcgggggttcgcccagcgcgctcatagcaggatccacagggccagggggccccccagtgtggaaagccggggccttaaagtgggtgttcatgctcagtccggtctcgttaaagttcctctcgccctggccagcgttcctgctgttgacctggggctcgaattggtccagcccaaacatacttggcggggggcagagggggatcaatagggcatgacagccggctctccgcggcgcgcctccggccagctactcgttccagcccaggattgggcgctccgggacgctcagcaccgcgggggctcagcgcgcacctccaccccgcctgatgtgagggacggggggcggggtattagctcctctcctgaagctccgattctgcccggggagggcctctcacatcttgcgaggccgcggggcctctaggagccgtgttggggggcccatgccccgggcggttgtcacagccgcgggtgggtctgcggggaggggacgaagccgcggatgaacggagacaaaaagttaagtggggggaatggggagggaagggggttgggagagcagagcgatcaccttctcaagtccgattgggtctgctggggagccctcaggacgccgcccgcagcctcccggagtccgtggcagagctgctaagggcaggggagggagacccttcaaagccgcggctggcgccgggcaccgacagagcggtccctccccccgccccccggagtccccgcgccccgcagcccgagcctccaccgagcacgatccgctcgcacaatccccgtaggctccgggcgagcggctgctgcttcttcagcgggtcggaggactggaggctccgctcggcatcgccggtgccggcccccgagccgaggacgcagaggggctgcgaggcggcaggcgccgggggctggagccgagggtcggggaaaggcgcggctcctctgctcggcagcgggtgcgctgcgttcggcgcgcagcttcgcggccacgtccgccgcctgccgcttctgttctccgccgttgggtgtctgagttcgccggagtctccgcgcaccgttgcaggcgcgggagggcagcgagacgaggggttgggtcgctccaaggctccggttccctgcctccgcgctgaggtgcttcgcgagtccctctcggacctgagggaggggggcgtacgcgggtcggggggccacgccgggcgagcaggctaaggcgttcccgctgcgctctcagagcccggaatggggggaggggggcgcgggggcagctctggggggtctgcgcacccctctcccgactagcgggggggctctgcgtggggcgttccgagggtctcggctcccctctctgtgtctgcctctcgcccagcgccgggagaagcagcaacaagttttgcatttcagcaatcaatttcagccattacatttgcaccaatcagcgccgcccaagttccgggctcggggcggggctcgctcttaaggtggtccggggtcctggctgccgaagcccccgccacgaacccgcactaccccagctctggggtcccctgcgcccgcctctcctcggggtccggcgtcagtgcgctgggggcgcgccccggcctccgggcgcatcgagctggagagggtgcaggcggaggagaatggcgggagcttggctttgttcacccccttcccattcacactgttcccaactccccacccccaactggagcgagacccaccgggtgggcagggggcggggaggtgggcggtgagcggtacggggcgctcagacaatgaggtcgccaggcaaagaccctagactcgccacggaaagtcgctaagtgtctactgcacttcggctccggggtgaacactccgttctccgctagcggcgcccaagttagccggccctgggattttcttggagggtaaagcagacggggaacctcgaagttagggttctcctcgagagacctgggcgcttgctcactcctccgtcactagggcgttcacaattgttccccttccccactgcctggggttcccggttctcaccggccggcgtgggctccaaggagtcacagtcactcagccactttatgggggagcacatcggttgagcccagtaaccggagcgacttgcgaggcggctgggaaccccgagaagacacgcacgaagagggcgcacccgccgggccgcacaccttgccctgggccacccggtccaagacccagaccctctgcgggtgcg

SEQ ID NO: 312 - DNA, 327 bp, Homo sapiens
  source 1..327: mol_type = genomic DNA
cgcctaccacgtggccgagattcccgaggctgcagccaatgagcagcatagtccgatgggggaggctggaagggccgttcacgagatctctcgggaggaatcggtaggagctgcccaactgaaagggacgggggaataacccggggcgagggacctgcccaggccctgccctctggggggaagccaggccaggggccgggggcatctctggggtcccaggtgagatgcgggtcgagggagccaggccacggttctccagactcgcgtggaggccacgagcgtcttctggaggagcgggcactgcgcggaccggcagactctggggcg

SEQ ID NO: 313 - DNA, 1949 bp, Homo sapiens
  source 1..1949: mol_type = genomic DNA
cgtcctaacgtactgtcgttcctgaatctcgcaggcaccgacactgccctggacactgtgagggggtgctccgtggggggatggatgaccggcagggcggactggagggcgggacggggagggctcggtccccccgcgggtgtagccaacagcctccccccgcagctcttcgcagcggtatcccgaggctgctgcaccagccttacccacctcgacgcttcgaggaacgtcttctcccgcacgtaagggggacctgtcggggccgggggaggctgctggaagccgcctccttgcggccccaggcccaccttcccacttcccccaggaagtcccgcgccgcgccggccgcgctgcagctcttcctcagccgcgcgcggacgctgcggcacctgggcctggcgggctgcaagctgccgcccgacgcgctcaggtcagtgtcggaccccggccacgcccccgcgggcgctcccaccctgccctggccttcgcccctccccgctcctgcttctgtcgctcccacaacctcccccagatcctggccctgcctccttcgttcgcaccctggagccccctgtcccagctcccgccaccccgtgtaacgtcctctcccagagcccttttggatggcctcgcgctcaacacgcacctccgcgacctgcacctggacctcagcgcttgcgaggtgagcgccggcccccagaagagaccacacattgggagaggcgctgggaggcggaagggcagggccgtgggccgcctgcccctccccactcgcggcctaagtgggtcccacttcccacctcccacctcccacatacagctgcgctcggccggcgcccaggtgatacaagacttagtgtgcgacgcaggcgctgtgagctccctggatctggcggataacggtgaggctgcaggagagcccatcctcgcatcatccactcgattcccaatccccaccctacccttgcaacttcgcctcgtgcgtgacccgagtcacccccaggcttcggctcagacatggtgactctggtgctggccatcgggagaagccggtccctgagacatgtggcgcttggaaggaacttcaacgtccggtgcaagtgagcccccaccctactcctgggcctcccagacaacaccccaccacccctgtcccccacaactgcggcccctgcccacagggagaccctggacgacgtcctgcaccggattgtccagctcatgcaggacgacgattgtgtgagttcacgggaccttgcagggcctcgggcaattagaccactttggtcctcctttctcttgttccctcagaccctgtgacctgccctcactgacccctgactccagccatcaatggctttctcttaaccccagcctcttcagtctctgtcggtggctgagtctcggctgaagctgggtgccagcgtcctactccgggccctagccaccaatcctaacctgaccgcgctggatatcagcggcaacgccatgggggacgcgggcgccaagttgctggccaaggcgctgcgggtcaactcgaggctccggtgggcggggtcagaggggtgggaccagcgggcagggggcgcggtggagaggagggcaccgggctaaggggagggactgaatgaggcggagcaaatggagcaggctgacgaggcgaatggactaggccgagggttgggtggggcgttgggaagctccgtccccgactgaagccaggcccggcccaggtctgtggtctgggaccggaaccacacatctgctttgggtctgctggacgtggcgcaggcgctggagcagaaccacagcctgaaggccatgcctctgccactgaacgacgtggcccaggcgcagcgcagccgcccggaactgacagcacgtgcagtccatcaggtgggcgtccccctcttcccttgcccttctctgcacggtaactccgtccctcg

SEQ ID NO: 314 - DNA, 319 bp, Homo sapiens
  source 1..319: mol_type = genomic DNA
cggatgccggcctcctgcctcggccaccgccctcctcttcaaggatgctgttttgcctctggtcgctctatcttatcttttttctccgcactcctggacgtctctcggacctttcgctctgacagtggcttgagcctcggccgccacttccgcccctgctacttcggggccacgtctcggtccgacgctgaggggtgctgccaaccgtgtcttctctgtgaacgtcctcacgatgaacgcccccgttccacttcccaccctggagctgctgcttccgtgactcacattccggccatttccgtggtgtgaggtacgggcg

SEQ ID NO: 315 - DNA, 776 bp, Homo sapiens
  source 1..776: mol_type = genomic DNA
cgttacattgcgattcattcaggaacccacgagacctctcccgcagcgtccccgctgctcaccgggctgcagacgagtgcgtcgcggtaccccccgaagagcagggcctgggctttgaggaagagacgggacaccccttccccgggggccagggcgaccttcctgagccggagcctcagcagggacacctgaagcacaagggagtggccctgagtgggcaggtgtgggcccctcgcctcccggggcaggaaggtgggaggggcgacactgaccacgtctggaggcagcgcctgcacgtcgttaaaggtcgtctccaaggtattggcgtccacgttcagcaccacgacgtcctccagggctttttctcgtactctctgcggaaaagcggggtcggccgctcagagcccgggagtccttggcagaggagggcatctggggatgtagaggaagggggaaccctcacgtccccgtcgtctgggtagatccacgctcacctcggcgagactggcgtgcactccaatgaggtagggcatgggcgcgctgcggaccgaggggacggggtcatgcaggcaccgcccccagggcctgcccacaggccctgcccctcccagctgccctcgctcaccagcagtagtccagcaggtgtgggggcagcgtggggatcagcacgtgctcccagcgcatggggtacaggagcgcgcaggacgcgtggacgcacgaggtcagctggggagcgatggcggggcgtggagtcagggcctggaccctctagcgcg

SEQ ID NO: 316 - DNA, 288 bp, Homo sapiens
  source 1..288: mol_type = genomic DNA
cgtgaaatcgccgttcgtgggcccacggctgcaccccgagatagaagcggaataaagggacgacgttaccttcaccagcctgagtctcttgggccgcgcccgcctgccccgactccaagccctacaggaaccggtttccctggttaggaggtatccacccggctcaaccagggtctcgacttccttggaaaagaccaaggcagccgtacgctgctggaacgccggagtgcaaaggcttttagggctacattgcaaccctcgggctcgatttcgccctttgggctgtcg

SEQ ID NO: 317 - DNA, 482 bp, Homo sapiens
  source 1..482: mol_type = genomic DNA
cgttgccgcctttttcgtactgagcgcgcagggcactacgcccaccactccccttgacccactcctcaagccctgataccggggcgaggccccttccttttctagtccttctacgcgccaggtgggtgggtttcgcggtgacgaggcgacacctctttcccctctggcgagcacgttaactggggaagctaccaaattctcacgccttagctgccctgcctccacctcccgcaagtccggttgcccctctaggcttctgcgcctgcagtggttccgaccctcgcctttagaactctgccgcttccgcctcttatttcgcgcctttgtttttagttttggaaggggcggagttgaaatggctgcgccgcggttgccgaagcaacgctttggttacttccgttggtttcaatgcttccgggttggcgctgcagtggcgtttccgactgtgggagcctcagcttcccagtcgtccgatgagcccg

SEQ ID NO: 318 - DNA, 3343 bp, Homo sapiens
  source 1..3343: mol_type = genomic DNA
cgaggcccaggcgagtggccaagacgcggcaggacaggaggatgcgcctggctgctctcggctgctggacgcggcctgcagtagggagagcgcagcccggggtaatcccactcagggctccgggtgcccagttctcccacttcccatcctggcggcctaaaggcaaggcctagaccctggagaggccaaagtcgggccaaaacaacagcgaagctgggactggaacaggcgctgggggcctgcgcatgaccccaggctcagaatgcgacactccagtgacgccggggaatgagttgagatggagccggagacagaggttggggttcaggacgaggcgcttgggtatgggatcggatctgagacggttgcggcgctggtgctttgggctggaccagactcgaacccagagcggagcgagggtcagagccagggcaagagtccgaagcccagctcagcgtccagcccgcgagagaccccaccatgaatgacgtccgagtagggttggaactcgaagcccgaacgcactggaaatgcagccggacgacggctgggatccgagtgggtccgggccagaaatggggacctcagagctccaccgaggcgctcgaggtcagaactgaagcctgagacttaggcaggacccgagacagagccagagacgataccgaaacccaatggaccgcgaggaccgaaggcagatccggggcgcaaacctgtgcaggcactggcgcgctgcctggagctctgggttcgcgccgctgagcgccggcaggttggaccagcgctgcgcgccggaagtgcactcgggacgccggggttctggaggagagaccacacgccgcaaaagtggcctccgaatgcgcccggggcctgccctcggcgacactgctggctcggcggccgcagcctacctgaagtagtccatcttgcagaagatctccttgttcttgatgtagcagctgttctgctgcctcagcgacgtgcgacacacggagcactcgaggcaccgcacgtgccagatgaggttgttgacctggggacggggcggggacggaggtcggctcagcgcgggcctctttcactccgggccccagccttcccggtctcatttacagtcagaggcgctgaagctcagaaggtgcatgcgattcccctatttttctcccgtaatactgagactcagggaaatggaaataaactcttcttatttttcagacggaacccgggggctcaggcagaccctaagtcttgcccaaagcttcgcagtcgggcagcagtggagccaggatttggaagtaagaggacctgcggtgcttccctgcagtctcctgcgctgcgtcccacgccccgacaacacgcacgcaacacctacccctgtctcaccttgagcagatatcggtccaggatctcgaggccgcagctggagcagatgttcttgcctgcagacggcacggaggaggcggcagagggcggtgagcagacagatggcgtgctgggcgtacatggggaggcctgaccctcgtccttgtccagagctcctgccagggcctcggcgtcagactgagcctgcggcgggggagagaaggagaggagcgctgatccgggcacccagagttggggctgccttggagacaaatctggggcccccgagcaccaattgacaacgaaatctccccaggacaatgcgccgtagactgaaggacagggatggaagggcctattggagaggaagggacggccccaacttttaacacgcgcattctgggtgctgggagcggttaaagccaacattccctgccggaaaaccgaggctcagagaggggcggcgcccgccgggagtcccccagagcgtgagcagcagttcgtggccggaaaacctggccaggtccccagggccgggccgctggaggactgggcaccgcaggcagggccaaacggactcaccatggcgggcggcgcggtcccttcaagacagcgggtggtcgctttgcagccggaccctggctgggccatcacctgggggagggggggagggaacgcaggcggcggcggctgctgaactggctccgcacagcctgagcccagcgcctccccgcgcctgttatataaaccggcgccgaacaatgagtcctaactttgtagtgggcatttaaagcccttccccacaaaagcggtgtctctctaatgaagcaatttgaatttggattggattttttccctctctctccccctctccctgcacttaacccgtggctcttgaagtaatcgcttagttcccttgcaatccaagcctctgaggggggaagaaaaacacgcgcacacacacacaaactacctgcaattagaaattgtcgtgaatgcccaagataagaggtagccagagtgtcaattccaactgtcaatcagtgagatccatcaggccgcccaaagaattgcaaatttgattttttaatggtagtaattaaaaatcgaattttttctctctccacccgcccccccaccccactccccttcctcgctcccttctcctctcggcacaaaatgcaaaaaggagaaaagagagaaagaaagaaaataaaaaagagatgggtgaggggcggagggtggttgaagaaaagaaaaccccgagcgcggtgagcacccgcccgcccgcccgcctgacacgcgcggcgaaattgaagcagctatattgacacggattcaggcgcctttcgagggccagtcggagggaaacccggagcaaaaagagagcgagatcggggcgaaacgggaccaaaaagagaaaagagaggcgctcaaggggaggaaaaagcaccctccagcccctcggcgcgccgggtaccggccccgcgtcgggattctcagcgctgcgccggcacaacccccggcgcatcggcgctatcagcgcctattcagacggacaataccggcctcgcctcctcttgattcgcctgtgtctttactaaatcgctttttgagaaattcctccaacatttgcataatgtgttcccgctttccgcgaccccccccccccgctcgcgcgcgcgctcccacactcacaagcacacactcacaggcgcaccctcgtacccctccttcctgcaaccgcccgccaccctgctcggcccggctgggtcccggaccctgcccgaccccggcccgggccgctcacccagtcgttcgccggctcacctggtcggtggcggggccgccctcggccggcagccggcagccctcgggcaacgccggggcggcgttctcatgcttccagtacatgggccggggaacctcgggctcagcgggcgcgcagcgcggagagctgcgccgagggggaccacagccg

SEQ ID NO: 319 - DNA, 2956 bp, Homo sapiens
  source 1..2956: mol_type = genomic DNA
cgcctctttcgcacaggtgcccgactcggagtccgggaggcgggccctctttccaggtacccagcccagcgtcagcctgggtccatcctccgccaccccacagcgcggggagcaggggaggccgaaagccgggcagcttggggggccgggataacccgggctgcagcaaccccgggcggcagcaggactggagagctggtgtcggacgcactgcagagacgtccctgttttcggcgcgtcctccgacacccagcagagagcttggcgctctccgctctgcgcgcccgaggtcacagcgcggagaaacctggcggggccccggactccccggcttgggaaaagcgatgactgccctgaactgctggggcgttcgaaatttccagggtcccgaccctccgtggggtacgcgcgacttcggcgcagatgtcagtccgctgccttccgggttgagggagcgaggactccagacgaccccagggccgctgtccaggcccagccccgcgttctccacctcgccacctcgctctgcggctccagcctggagatctacggacttcattgcgatctcttgcgcagtgtagtcgccctctatccctagccccgagtcccgggtaacccaggggccccggcgaggcgagtttctttggggcgagatgtcccgagccgagcaaaggggccgcgcattggacggggctcccaagccacagccctgggggccttgaggagtcgtggtacggtccagggcagtcggggctgcggggcccgtgaagccagacgaggctacggacttagggtccctgcaaccaagaccgcgagcgactgtggactcgggacggatcgcacaaaggaggaagggggaatcagaggggcgtccggtccaggacgtgtcaggcctgggcggggcggaaaactgacaccagggcgaggacaaggatttcgctcctccagggctgccaggggaggggctcgccaccgtcactctccatgggctctggccccgacggtgtttaaagggcggagggctgggccgggctggtcgcacccgggcgctgctggcggccaagctggatgggtcgccagtgagtttcggtgcggcaccgctggcccaggcccgggcgcggctggacatggccacctactgcgacgacctgggcccctcctcggccccgcccggccaggcccaggccaccgcgcaccccccgggctatgagccaggggatctgggcgcggtgggcgggggccccctcctgtgggtgaacgcgccagcgctcagccccaagtcctacgcttcgggtcccgggcctgcgccgccctacgcggccccgagctacggggctcccggcccgctcctcggcgccccgggcggcctggcgggcgccgacctcgcctggctgagcctctccggccagcaggagctgctgaggctggtgcggccgccctactcctactcggcgctcatcgccatggccatccagagcgcgccgctgcggaagctgacgctcagccagatctaccagtacgtggctggtaacttccctttctacaagcgcagcaaggcgggctggcagaactccatccgccacaacctgtcgctcaacgactgcttcaagaaggtgccccgcgacgaggacgacccaggtaacagcggcgcgccggctcgctccgtccaggactccccatcttcccgccgggccgcgggcactccgggggtgggagcaccttagacaacctaatttctccctgcgcggttggggatacccggggaggagggagaaggggaagaagtgcttgggcatggacaggttctcagacagcggctgagcccaccacaggcccgcgtttacagtgcggcggtcccggggctctcaatctctgtgcgctccttcggaggaagaagcgggagagccagcaagctgggtgaccttgggagtgactcaacttccctgggcctcagtgccctacctggtaaaatggggaagacagcgcctaccccaaggatgggcaaaacaaataggatgcccgtttggaaagctctggagcctacctgggggcctggcgtggtctgcgctgtccttggcagagcggctggcggagtcccaccattagcatctcgcaggccttccttcaccttaacggggctcggcctttcccagggctcccttcctcctcctttctggattgtcactttcttcccagtcctgctgtggaggcccatcgggtacagagtgcccgtcaagggtggaatgggggcgaggactcagaccctctgtaagggccagcaggggatctttctgaagctgacctcctcgctggctcacaccacggcccgtcaaagctcgaaccttctgaactgggctgtttcttcttctgcaggtaaaggcaattactggaccctggaccccaactgcgagaagatgtttgacaacgggaacttccgaaggaagaggaagaggagagctgaagccagcgcggccgtgcgctcgggagccaggagcgtgggaggggccgaggcgccagcgctggagcccccgagcgcggcttgcctggacctgcaggcctcgccctctccatccgcacccgaggccgccacctgcttctccggtttcgcttctgctatgagcgctctggctggcggccttggcaccttccccgggggcctggcgggcgacttttctttcgggaggcggccaccgacagtcgccacccacgctccccagacccttaacccctcccctggcttcgcccctggccaccagaccgcggccgccggcttccgcctcagtcacctcctctacagccgggaagggaccgaagtttgaagggaggctggaggctagccgggtgcgggtccagaggtgctgagctcaggcctccggtttcccctggtgacagccccacgtgttggcgttgaaggccttggtgccctgggaggaagcgcagagccgggggcggctcg

SEQ ID NO: 320 - DNA, 791 bp, Homo sapiens
  source 1..791: mol_type = genomic DNA
cggggtgcggggaggttgagagcgcggcggccgctgccagcaatcgaggagccagcggcgcgtgtgctgagggcccagctagcaaaataaagagggttttcagcggagcggcggctcaggcgaggctgggggagccggggagcgcgtcccgccatttttccagccgaggtcagcagcgggtgaaggggcggagtccgtggtgttcccaactgcccaggcgggcggtgagatggggcaggcggttccgcaacgcggctcaggccccagcgccctgcctgcaggaggccgcttctttctctgttgccatcgggcgccggtttcctcttccggggatgcagggcagctgggggggctctgcctctgtgcggctcttccgtggttccacgctgggctccggggtgaccacagtcctcagcagcccacaccctcagcccgaagggggcccaggccccgtgcccagaggatccagagagacagccgggagcccagcagctgtggcagctgcccccaccaccaccccggggctggccaggtcccacctcctgacgccacacggtatgactgggccctcctggtcagtgcgtcgcatgtaacgccttcaactattggggccggtgttggagcatccgtctctgcgtccccacctgatgacaagctccggggagcaggagtgctggccaagcccacgcacggaccaagcccacgtcagactccgccagaaaggcaagaagggcctccgcatggcgtgacggatgggtttatgggggcattccatcccacccggacgctcg

SEQ ID NO: 321 - DNA, 4184 bp, Homo sapiens
  source 1..4184: mol_type = genomic DNA
cggagcttgagcgccagaaccacgcactgaggtcgcccagggtggggaagtggggggccgggggaggggcggggcatccggcggggaggtctcccccaccgcatgcaagaccctttgcccccccacaacatacactgaggctaaaacggggatgagagtcacacagagcaggagcgaaatctttcgctctccgacccgaacggcgcgttgctcgaccccccccttcctggagccaggggctggccgaggaaggtggctccgttcttcccccgtggccagtgcggggtctgcggctgcccggcgaggttccggcttccccgggactggcagggggagccccgagaactgcaggcccgcccccccttcctcctcctccttcccctcctcccccacgtgtcctccctgagcccagcagcctgatcccagcaccccccgccacctccctcctcccgcttcgcccgcttccccggcggcgtggagaagagcaaagttgcgacagcggccgggggctctgcccaggtaaagggggcgccgtgaggcgggggtggtgggggaacggtgcggccctgcggcgggggcggggggaggataaagaaacttgcttcccgcggcccccaccccgcgtgggttccgatcttgcgtggaacgagcccgcgtggcgagtcgcggcaacactgccgggaccgaggccggcacctggacccggacccggaccgcgcgcttgggccggctcggcgggcgcaggcagcgggccaggccagcacccaggcaagggcggggtgggtggccgtacaccggaccgaatcggggactccggtggcgtgggcgcgggccgaaccccgaccgggccgagttgggtggcgaagtaggacgtggattcgggggcggggcgcggcgggagggggaaaccaggcctggccagactggcctgggcgtgggggcggggggcgagttcggggcacttgctgggctctggaccgagcggtagggcggggcggcgctaggggctgggccggacccgcagggagagggcgaggggagggagagccgggggtggagtaggggcgtggagttgtgagcggggtctggcctcggccgggggcgaaggtagatcggcggggccgcgagcggagggagggaggcccgcggcggcgcggcggcagcgaaggccagcttccgcggagtttgtgcccgggcttcccgggctctggccgcctcacgcgcacaaatggggctaggggactgagtggtaagcaactccgagtgttagacggtgatcgggcggcgattccgggaaaagcgaggaaagacacagtctgcgattgtgccgcaccccccacccacctcttagcatctggattctgctctcgtagtgggggccgcggaccctccccgccacagtccttttactctccagcactcccaccgccttcccccttcttcagccatctgactctcctaggggtatgtgtttttgggggtggacggcgacgggggtgggggatactttccttcccgtggcggcaacctagttcgtatcttattcctacaaggtagcgtcgcttcgagtcgcatggacattggggtttagataatttaccccccacccaagcgatttaatggcttttaagcaaaaggcgaatacctggatttccatgggagggatgaggtttgaagattgggattcacccctagctcacttccactaccaacccctctttcttgtgagccacttccacgggtattcaaatctgggggggaggggggacgggagtattaatatgcttctctatttctacacagggtcggcgtggcgaaggacggctagccttggagggaaagtagccaccagtccaactcgggtcgcccccaccattatttcggtgagggggcaacgctaagaaggggatactggtgggtaggggcccagtataagggagggggctgggtagtcgtccccagaaggactccgtgtcttggcggggagagagagaggcgagccccggcttcaggggaggggttccggggagggagatgggggggtggcgacgacggtgggggggggagcgggaagaagggggagtcacgccctgcctgcaaggggagcctccctcaggcccctgggagaaggggactgaatgcgtctgttctccgctgcccggcagaggagatctggggtctcgagatttgcacactggatcggcaccagtggcctgagaaagtcaggtcggggcataaggtggagaacgttggaagaaagagagagaaccctttggccgcggctcactgaccctctcccttcctcaacctcccccttcgaggggcgtcgaggtggggggggacgtggggctcctgcagccccgcagattgagcgcctccgcctctcccaggagtctagctttttgcccggcttgctgcagccactgctgcctccctctccggaatccgctcggtcctctcagcgatcccctcctcctctcctcccctcccagtgtcaccggggctgcttggctctgcccccttcgccgccgcttgctccctgctttcgtccctccctctcttgctcgctccctcccttcgcctcctcctccccattcccccccacccgcgtaggccgcattctgggagttgtagtccggtggagcgggggttggcgccgcccgcggacccagccggactccacttcccgtcgagccctgcgaccggcacccactccaccaggcttcgctcgcacacagacacacacacacaccgctctccccctcactctttcgctcgccgcggctgctgccagtgtgtggctctgtctctcctccgctttgctgagccctcccttcttcctctcagttcctagagtccgaccgccgccgccgccgagagagaggagaaggaggtcggtggcgataaggggcggaggggggcatcagatcggggcagtattaggcgggtggcggttaagagggagtaagagggagcccgggtggcggcctgagcctccccgcggagccgacccgggaacaggtgcgtcttttttctctctcccccaatccctccaccccttttgactccccggctttttcgtatccccccacccgcttctttctatcttaagcctttctccaaccctgttccttcttttccctgcggccgctcggcttgccccccgggggcgccgtcctcttcgtgtccccggacgtagcgctccctggagctgggtatgtcttcgcccctcccttcgggacagctcccttccacttcccaccttcgtaccccatcccctgacttgccctacccccttctcgacccccttctctcttagccaggtgagactcgttcgccacgatccaagagagtcttcttcccggctgggcgggggtctccatggaaacgggggtcggttgatcctgggggggaaaccataggaattaccccactcctcggaaagggaggtggaggctatggtcttgttaactacctgactccaacccccgatccaaggatccgacgatccgaagggaggctggctagcagggcagggcgagaaaaggcttttttttggggggggggggtgtccgtgatgaagtgttgaagagaccgggagaaaatgccctccaacctcagaggggaggtgcgacgggtgctcttcagggctgctttcttcccggcgctggggtgcgggaatcgtagggtaaggggtacccctaggtggcggcggggcctgggttcgggagtgggagtgctgtgatgggggcaggggcaattatccgagtccctggaaaaaaggcgggggatgacttcggagcgccctggctccgctgccctcgctctaggggcgggggccgccgggccgggcgcggcggggagggggcttaaattgaaggaagatgagatcgggggcaattcatactagcaccggcaagccagaggggagatccggcgtgcaagccctccacccgcttcgcgccaggcctggaggcaaggggtcggcgagcgaagccgagggctggaaggagggaggctccggttacggggccgggtttcgggatcaggggcaatcccccggcccttggagaaggagggagagggatggccgcgaaccccagggctcccccaccccgggtcctaaggggcgtgagggacggacagcaggcccagcggagctggattcgagcggatcgcggccg

SEQ ID NO: 322 - DNA, 514 bp, Homo sapiens
  source 1..514: mol_type = genomic DNA
cgagttggtgcgggacgtgccgcacggcgtgcttctgctggacaccttccacaggcttgcctcccgcgaggtgcggctgccccgcggcttcctcggcttcccgtcccgcgtgctttcctggagtccctccttccggaggccctcctgcctccacgtgtgcccttctccatgtccagcattcgggcgcctcttgtctttcttctgttccctggcttcggcgtccccgggagtgtgactcccgcagcggggtgcagctttcctctgggatgagtgaccggagggaacccgccttcccgggcacgtcgccagcctcttcctcttcttccctaggctatcaagcggacttatgacaagaaggcggtggatctctacatgctgttcaatagcgagctggccctggtgaaccgtgaacggaacaagaaatggccagacctggagccctacgtggcccagtattccggaaaggcgcgctgggtgcacatcctccggcgtcgcatcgacagagtcatgaccg

SEQ ID NO: 323 - DNA, 2097 bp, Homo sapiens
  source 1..2097: mol_type = genomic DNA
cgaggaggctgacccggagctggagcccgcgtcggggggagagcaggagccgcggccccagcaagcccaggtaggcgggagtggcccgtggctgctctcaacatccggagcggactccgggcggggagcgctcctgcccagggctgcgagccgcccgcgacccagggcgctcggggcaggggtggggaaagaaggggcgccccgtcacttgccccctctgcagaccaaggccgcgtcccagatcctggagaatggggaggaggccccggggcccgacccctctctcgaccgcatgctcagcagcagctcctcggtgtccagccttaactcctccacggtgaggcgggagggaggggacccgggcggccggggggtggacccgttccgatgcgtagcccctgcctgcccctccctcgccgcgggacccaccgctgcagccccccagcctgccacctatgacccgggtctgaagcctccgcgctgcccgcggccccgacgtgagccctgcgagcggccctgactcccacccactcccgtccgcagctgagcggcagccagatgagcctgtcaggcgacgcggaggcggtgcaggtccgcggctccgtgcacttcgcgctgcactacgagccgggcgccgccgagctgcgcgtgcacgtgatccagtgccagggcctggccgccgcccggcgccgccgctcggacccgtgagtgccccgccggccaagcggggcgcggctgtcacagcccagcccaccattcacagggtctcggcctcctcgtcctcatcttcaaaatgggaacaacagcgttattgggaggcgtgcgattaagcgagacaatccctgtaaagcgcttagcacgaggcctggcacgtgttcgggatggtggctgggggagcccacaggcaggggagaaggctctgggagggcccctcctcacctcgggttctcacctccccagctacgtcaaaagctacctcctcccggataagcagagcaagcgcaagacggcggtgaagaaacggaatctgaatccggttttcaacgagactctccgggtgaggctgtgaccacgatgcggttccccgttaatgaactggacgcccccttcctgcggggctaggtggcaagggcagccagtaacgtcattgcccggaggatcggcggagggggcccattaactcgttatccagtgttgtcagcctcgtggtgggcgtggtgatagtgcaggtccccattaatgcccttaggggctccccagaattccatcatggtaggaacgcggtaggacctgcctcagccaactcgcgcagcatctacgcgggccaccagcagtgctccactaaagctcacctcctgtcctcagtactccgtcccgcaggccgagcttcagggccgcgtgctgagcctgtctgtgtggcaccgcgaaagcctgggtcgcaacatctttctgggcgaagttgaagtgcccctggacacgtgggactggggctctgagcccacctggctccccctgcagccccgggtgaggcagccaggccgcgtggggagacctgcggcccgggtctcctgcatttaccccaccaggctctcccgcagccccctcacaccccgccttcgacagaacctcccctcaacctcttaacctcatggccccaggcgaagcccggccggccacggccccttccccgagggcgctaggacccctaggttctgcccctgcaggccccgccgtctcttctagccgcaccccatccgggtctgcagaccccaccctcctgaggcccctttccattagcccctgctccacgataagcccgcctctcgcaggtcccaccctctcccgacgaccttccgagccgcgggttactcgccctgtccctcaagtacgtccccgccggctccgagggtgagtgacagccggagaggccaagctggacacgccctgaaaagcgggagactccagtccccgggtttgggggcggtggactccatccgtgtgcgggcctgagccgagcctctccgcaggcgcaggactgcccccgagcggggagctgcacttctgggtgaaggaggctcgggacctcctgccgctgcg

SEQ ID NO: 324 - DNA, 2334 bp, Homo sapiens
  source 1..2334: mol_type = genomic DNA
cgcccccttccattccccgcccgcttccatcacttggtgtcgcccaagcctgtggtcgcccctagcaacgccctccctcacttcgcagtcggtttcccctttcatcatcgcccccccacgtcccgctcttggtctctcccgatcccgcgtggatccggtgcttgggcgcccccgccaacgacccccgcgcacctcagcgttggtgtcgcccggttccccccgcgcacgcgcaccgtggtatttcccgccacgctcctacaccgccccccccaatacctggttggtcacgtcccccgggaggccccggatcagtatcttgcggcggttacggaactggcgctcggtgtgttccaggcgtttccggatctcttctggatctagaggcggcagctcttcttccggcgcccggcgctccgcggcatcgccggcttcgacttcggccccagacttagggctcagcgggggccggtgagtaacggacacgtccgccgccatcttgggaaacccggcgccttctgggaccagcgagccggggcggagcggcatagagcggcaacgagggcgcgcccgtcgattggctgggagaaaccccacctccttcccgccccccttgccccgttggctaagccccagcccacctctcaagagttcaagctgcgtctgcgcgccacaggccccgcccccttccgtcgcagcgacgctactcagtggatgtgcacctgggtgggagagacttgcagcgcgggtggaatcaaaccacagattagggagtttgaaggctttattggtgcggaatctgagggcacagccaagcccccgccaactttgatcccggatcccagcgtcactcagctctggacggttcttcccccattgcttctgtcggctgcatagacgtgaggggcagataggtgctctcctccctaacatggtaactgccgctccgttggtgctccctgaagacgtacattaaggccagtacgatagtcaccacgcccagggtcagtaacaccgccacgaagacggggacaaagtgggagctcccagctgtgcagagaaagcgctaagtcaatatgcgtcccttctgtctccaacccccccgccccccggcttaccggtccagacccgcagccccgtgttagctcaccctcaatgtccatcaccacgaccagggtgtatttgcctcgtgagctggacgcttggcactgataagtaccattatgtgttacgttgacgaagaacgggatccccaccggcacctcccggctggagccttccttcaaacaccgcagctcggggtacgggttgcccctggcttggcactgcaggacgtgtctcgttttatctttccatttcaagtgctgggggcatgtggctcggtcaattttgggaccatctgtggaaccaccatgtgtgatcagacacccaacacacccgaggcacagtggtgcagaggagcgtctaatctttccagggcaggggtggagggattaaaggtcagggtgaccgactcacacaggactcgcagctggacgctactgttcctgtgcaagaactcgccgtccacctcgagagtggcactgcagaagaagctgcgtccgtcgtcactctcggtagcatttagctgaagttgagctggctgccccggggccgcggccggaactccgtccagcgtgacctggactcgagccccagccatgcaactcacggtcactgtggacccctcatgggcggtgggctcgctgaggttcacaatgggtcctaggaagcctaaaggcggggcattgcccaggagcttaatgaacaggaccttcctgtgggtcaagccgctccctccgccctcccctttcctctcgggatatccgggccacgctttcggccgttcaagcctcgccctctttccgcgctgtgtccagcttcgggcactcaggcccaacccacgttgcaactgctattggggcaagccaggccccaccttttcggctagtctccgccccctctgccacgcccccagactgctgaggccgcgcccccttcccacgcctcctcttactaaagaccgtcaagttctcccgggcctcccgtctctcgccccctagggtcacgttgcagacgatctcccgggcaccctcctgatccgcgcgcgccgtggctgtggctgtggccgttagcgtgtccccgtggttcatgactgtcgcattcagcatctggtcccccagcgccaggtagacctgggcctctgaggctggaaaaagcccgtctagggtgcagtccaccggccacgacgtttccacctccaagaaccggggggccacgaggcgcgggggggtcacg

SEQ ID NO: 325 - DNA, 1481 bp, Homo sapiens
  source 1..1481: mol_type = genomic DNA
cgcggcgggccttccccattggccagccaaacacaaccgactgcggcagccaataggagccgctctcctgaacattcagaggatgggtgcgcgtggtgtgataaggggatgttggcgcctggtacgcgcccccctacatctggcaagtctgtaacagaaacagttacaccccctacacacgcacacgcgtgcccgctggctccctcctctcagctgtacaggccccggacaacccactgcgtgtcttggcagcggcctcccaggctgcctggggcccgcgagtgcacctgctcactcattaagacagagcatccctttatcccaaggcgcgggtcgggtagagggtgggggtagaggggacggggtagcggggaccgggaaggcgcggctcagctgcccgctgctcccgaggtgaaagaggtgcctccgcccccaccccatccccgcagagttgcgttgctccagggaggccgaagtgatccagtgatgtcttaaggtgcagctctttgtcctccccagctgtgctccctgcccgctgaccccgtgcgctcgagccagtgggggtggggagcccgaaacctataacataaacgggggcaaggagaaagaagtctatcccggggtggcgtgtgccgctgtccccaccccgtctctttcctcctgaggcttcttccctcgggtccgtcccgggaatgcaggcaggggagggtgtgggccgaggcgcggcggcggctggagcagcgcggtagggtccttcgccagagcatccggtccgagggcgcacacaggcagaaggctcgcggctcgtccactctcctccctctctcctcctctccctggcttttgtgttggtgcctccgagctgcaaggagggtgcgctggaggaggaggaggggggcccggagtgagaggcacccccttcacgcgcgcgcgcgcacacggtgccggcgcacgcacacacgggcggacacacacacacgcgcgcacacacacacgcacagagctcgctcgcctcgagcgcacgaacgtggacgttctctttgtgtggagccctcaaggggggttggggccccggttcggtccgggggagatggcgcagcccatcctgggccatgggagcctgcagcccgcctcggccgctggcctggcgtccctggagctcgactcgtcgctggaccagtacgtgcagattcgcatcttcaaaataatcgtgattggggactccaacgtgggcaagacctgcctgaccttccgcttctgcgggggtaccttcccagacaagactgaagccaccatcggcgtggacttcagggagaagaccgtggaaatcgagggcgagaagatcaaggtgatccagggggtcaggtccaggaagggtgggacccgggaggggacctcgcccgaggcatagctctagcggttgtcgtcgtccagcgtccagcgcgtggcggtttcgcctcttgctgagccgaggaccctcg

SEQ ID NO: 326 - DNA, 1703 bp, Homo sapiens
  source 1..1703: mol_type = genomic DNA
cgcacgcccctcctgtctcgcgtccgtccctccgtgcagccggggcaggaccctgcaggggccttgcgggccggaggtgccggcttcctcctccgtcccgtcccccccgcggtgactctattcaaatctccccccttcccgctgccccaccggactcaggggctggggtcgtagcccggcgggcagcggtgttcaccttccgcataaacctgggcttcctcgcggggcagcccccgggccctgtgctgggtcgcagcctcgggcggttccctcggggcgggacgtgtccccgtccgtcctcgcccgtccccgccgcccccgggtcccggcccgccccgtcctcgcagccgcaggtccgctgcggcgcccgcccccctctcccctccccgggccttgccctgctcgtcgctccgccccctgcccggcccctgcccggccccgccccgcgcccgccccctgcccgcgcccgccccgccccctgcccggcccctgcccggcccctgcccgccccctgcccggcctcccagcggcggcggctgcagcgcggcggtccgagcgggtgcaccgcggcggaggaggcagcatcccgcggcgctgacggtcctggggagagcatggcgccgaggtgaggccggggctgcgggcaggggttgggggacgccaagatgcggctgcgggggtcgcgggggtcgcggggctcggccgggtcctgcgggggtcgcgggggtcctgcggggtctgacccgtgcctgtcccgcgcaggtgcccctggccatggccgcggcggcggcgcctcctggacgtgctcgcgcccctggtcctgctgctcggggtccgcgcggcctccgcggagccaggtaagacccgggcgggacgggaaggttcgcgccggtgcccgccggcctcgccgccctggctggggtccccgcccggctccctcacccccgccccggccgctctgggttcagccccggccctgcccacgcgcgcaggaggcgccggagccggacggagcggggcggggtggccggagtcccgcccattcattctccccgcggggtccgggccgggaggggtgcgcggtgcgagcggccggagtccccgcgcggggaccgaacctccgcggccggcggggtcgtcggtgccgggagggctcggggcggggccgcggggtctcgggagacgcggcctctgtcgccgtcacggcctcgtccccgcagcgccgggtcccgggactgaccgcgggcggaaggcggcggagcgcggggcgtcgtctccgcgtttccaacctgttgaggctcctcgccgcgggccgggccttgtgcgcctctgtccccgccgccctcaggcctctgtccccgcgcccgccgtggtgtcgggttgcgcggtcccctttgatgccaaccgagcgccgcgcggtccgagaacaatgcccagaagccgctgccccgccggcctcgccgcttcccgtgcgcggcaaacggtgcgcggggcccgtggggaccttcctgcctccaaccccgggacccagttccccaccacctgctaaccggggcctcctgcctccagccagcccagcagaggcccgggggaggcggcggggacgcggaccccaggggagccccgcccaaagggcctgggtgccttgtcgcgggtggaatcccacccgggggtccacgcagccagcgcctgcctccgactcg

SEQ ID NO: 327 - DNA, 699 bp, Homo sapiens
  source 1..699: mol_type = genomic DNA
cgaggacagggcccggccccaccccggggcgagtacgagtttggaagcagtgagggcgcagagtggggctccatctggggagaggcctctctctgtcacccggccccgtgcggcagttcagggtcccagcatctcccctgcactgggaggcggcagccgtgcccggggggcgcccgtggtgggcgggtgcaagagagaagggcgtcgggtgtgcaagcaccaccggcctcccattaacagcctgcccgggctccgcccacaggagccactgtccacgtaacctgcgtctctttctttcaaggaaaagagaagccagccatggccaggaccagcagcagggccccctgctcacccacctcggtgtcggatgtggactcggacgcactgtcacggggaaacttcgaggtggggtttcggcctcagaggtccgtaaaagccgagagagcgcagcagcctgaggctggcgaggacagacggccagctggcggggcctcagacgtggaggcagtgacccgactggccaggtccaaaggggtcggcccagccttgtcccccggcccagccggattccagagctgcagccccggctggtgcagcgccttctacgaggccgactgcttcggggccgacgtccacaactacgtgaaggacctggggcggcagcaggcggacggggccctgcccgacgcccagagcccg

SEQ ID NO: 328 - DNA, 1442 bp, Homo sapiens
  source 1..1442: mol_type = genomic DNA
cgaatgcagcatccctgcgctcggcgccctgctattggccagagatgatctcaccgcctcccggcctgcgcgcgcctgattggccggtgcggggatgctgcgctccgccgccggcggggcagtccttcaggcgggctccgcgagcgtgtgtgagcctgggggagcgaagagccgacgcgcctggcatctcccagggggctcagggggcaggagcgcggagacccccggacagggtcttaaccccttcccgccagcccgagcccagcatcccctccagctcccctcggcgtcccaaccccaagcaaagccatccccaaaggatgctccgcagagaggcggggtccgggtcctcccctccacccccttccgtttaaccctctcaccgccgcggcgccgtcctgcactgcccggccagctgggctgggagagacccgcgccccaggcatgtccaagcccacctagagggcgggagggtggcgggcgctctggccccaaccctggcatccgaagcatcccccgcgtttcctgctcctcactcagtcttcagatcccagtccggcgcccccggtacaatggggagccagggtgcccaggtcagccgcaagggccagcgtaccaggcggaggaccggcaggacgccggagaccccatctcccagtcccccctgccccagctctctccctcctgcggaggaggcagaaacggaccggcatctaccgcagcccagagtcccaggagtggccgccgaaccctcaccccgcggagcgcctgggcgcccagaggtgaggctggggtgaccccgccccctccccgggtcggccgggcccagcccagcccgaccctgccgggcgccgctgagctgcagctccccggctggctctagggccccgggcggagcggccgggggtcccagcccggcgcgcgtggcgggggccgagccgccccctcacctgcacctgcacgaaggagccgcggtagaagcagcaggcggcggccgacagggcactccacaggctgcaccgctcggtcatggtggggccaggctgccggggccgggacggtgtgggggggcggtggccggcggggcaggggcgcagggaccaaccggctaggcaccggctgcgcgtgcctctcagcggcgcgattacctcatcgccttgtcgggggaggagcggggaggcggggcggggagaccccaccctccagccggccaggggaggggagggggccattgttcctccctccgcccgccccccgctcccagcccctcgccccccgccctccagttccgcgcgctcgggctgcctggagctgcgggcgcggagcggaggacctgccaaggagggtattaccgggggcagcgggatcgggtggggacccatgggccagcgaggggaggttctgcctcccccactcgccgcacctctaacagaccgggagcg

SEQ ID NO: 329 - DNA, 690 bp, Homo sapiens
  source 1..690: mol_type = genomic DNA
cgcctgcgcgagtcaggacgagcgcttgccccagatcttgacgtttcaggcggcccctcctaatccggaacacgactcacattccgttggtcttgccttgacagacgtgaccctgacccaataagggtggaaggctgagtcccgcagagccaataacgagagtccgagaggcgacggaggcggactctgtgaggaaacaagaagagaggcccaagatggagacggcggcggctgtagcggcgtgacaggtgagggcgggcccgggagggctcggtttctggagcggctgccgggcacgggcagggagcccggaccgaaagctcagctccaggatggctgcgcttgggccccggcgttccctgcccggaaccggaggagtggtttgacccggggcgagaccatcgtcgacagcgggggtggggtgggtaacaggaataggcgggcaaggctgcgggtgatggtttcgcccgctgctagtggtggtgcgcctgcgcagagccggaagccctttggtaggcgggacccgaccgagtggtgccgggattccgtcttaacccccgctaaggtgtccaatgacctttccttaccaactgcggggagtgtgtggaaacgcggttcactccccggtttccttattctggatcagtgtctgaccgcccccgccgggagaatgtcaggattctcgcctct